(19) United States
(12) Patent Application Publication
(10) Pub. No.: US 2024/0252488 A1
(43) Pub. Date: Aug. 1, 2024

(54) MLKL BINDING OR DEGRADING COMPOUND AND PHARMACEUTICAL USE THEREOF

(71) Applicants: KOREA RESEARCH INSTITUTE OF CHEMICAL TECHNOLOGY, Daejeon (KR); HUONS CO., LTD., Gyeonggi-do (KR)

(72) Inventors: Jong Yeon HWANG, Daejeon (KR); Hyunjun KIM, Gyeonggi-do (KR); Hyowon MUN, Seoul (KR); Hyun Jin KIM, Daejeon (KR); Jae Du HA, Daejeon (KR); Sung Yun CHO, Daejeon (KR); Chang Soo YUN, Daejeon (KR); Pilho KIM, Daejeon (KR); Jisoo LEE, Gyeonggi-do (KR); Joon Gyo OH, Gyeonggi-do (KR)

(21) Appl. No.: 18/246,773

(22) PCT Filed: Sep. 27, 2021
(86) PCT No.: PCT/KR2021/013161

§ 371 Date: Sep. 27, 2023

(30) Foreign Application Priority Data
Sep. 28, 2020 (KR) 10-2020-0126423

(51) Int. Cl. A61K 31/497; A61K 31/4439; A61K 31/444; A61K 47/54; A61K 47/55; C07D 401/04; C07D 401/14; C07D 403/04; C07D 403/14

(52) U.S. Cl. CPC A61K 31/497; A61K 31/4439; A61K 31/444; A61K 47/545; A61K 47/55; C07D 401/04; C07D 401/14; C07D 403/04; C07D 403/14

(57) ABSTRACT

The present disclosure provides a compound of a specific chemical structure binding to MLKL or having an MLKL inhibiting or degrading activity, or pharmaceutically acceptable salts thereof. The present disclosure provides a composition comprising such compound or pharmaceutically acceptable salts thereof. The present disclosure provides the pharmaceutical use of the compound according to the present invention, salts thereof, and a composition comprising same for treating or preventing MLKL-related diseases. The present disclosure also provides a method for treating or preventing MLKL-related diseases, comprising administering to a subject in need of treatment an effective amount of a compound according to the present invention, salts thereof, or a composition comprising same.

### TECHNICAL FIELD

This application claims priority based on Korean Patent Application No. 10-2020-0126423 filed on Sep. 28, 2020, and all contents disclosed in the specification and drawings of the application are incorporated in this application.

The present disclosure relates to a group of compounds that competitively bind to MLKL (Mixed Lineage Kinase domain-Like protein) and/or have MLKL inhibitory or degrading activity. The present disclosure also relates to pharmaceutical compositions comprising such compound. The present disclosure relates to useful methods of treating MLKL-associated diseases using such compound. That is, the present disclosure relates to medical uses of the compounds according to the present invention for treating or preventing MLKL-related diseases.

### BACKGROUND ART

MLKL (Mixed Lineage Kinase domain-Like protein) is a type of pseudokinase protein and consists of four α-helix bundle structures at the N-terminus and a pseudokinase domain at the C-terminus. Unlike general kinases, MLKL lacks ATPase or phosphate transfer activity.

As shown in FIG. 1, when necroptosis is induced by various signaling substances, phosphorylation of MLKL protein occurs in the final step of signal transduction, oligomerization proceeds, and oligomerized MLKL migrates to the cell membrane to form pores, thereby inducing necroptosis. MLKL protein is currently attracting attention as a new target for various diseases related to cell death and inflammatory response.

Necroptosis is involved in various diseases related to inflammation or tissue injury, including traumatic brain injury, stroke, bone marrow failure, (acute) pancreatitis, atherosclerosis, ischemia-reperfusion injury, transplantation, infection, chronic obstructive pulmonary disease, remote lung injury, hepatotoxicity, (alcoholic and non-alcoholic) steatohepatitis, remote liver injury, Crohn's colitis, ulcerative colitis, and terminal ileitis, and the ripple effect is expected to be great when developing an excellent treatment related to this (H Zhao et al., Role of necroptosis in the pathogenesis of solid organ injury, Cell Death and Disease, 2015, 6(11), 1-10). MLKL inhibitors are expected to be useful for the treatment, improvement or prevention of these diseases by consequently inhibiting necroptosis.

For example, it has been reported that phosphorylation of MLKL, total MLKL level, and necroptosis are increased in non-alcoholic steatohepatitis (NASH) patients and NASH-induced mouse pathology models (Afonso et al., Necroptosis is a Key Pathogenic Event in Human and Experimental Murine Models of Non-Alcoholic Steatohepatitis, Clinical Science, 2015).

In addition, the expression and activity of MLKL were greatly increased in the liver tissue of autoimmune hepatitis (AIH) patients and AIH-induced mouse pathology models, and consequently increased necroptosis was observed (Gunther et al., The pseudokinase MLKL mediates programmed hepatocellular necrosis independently of RIPK3 during hepatitis, The journal of Clinical Investigation, 2016.). And, it was confirmed that when MLKL gene was deleted in an AIH-induced mouse pathology model, liver damage levels, ALT and AST were reduced to normal levels and cell death was inhibited (Gunther et al., The pseudokinase MLKL mediates programmed hepatocellular necrosis independently of RIPK3 during hepatitis, The journal of Clinical Investigation, 2016).

Therefore, it can be confirmed that necroptosis and MLKL are important factors in liver diseases such as NASH and AIH accompanied by cell death and inflammatory responses, and that MLKL protein can be a new target for the development of liver disease treatments.

Meanwhile, Catalyst Therapeutics Pty Ltd confirms the anti-inflammatory treatment effect related to psoriasis through MLKL knock-out mouse model, and is researching and developing MLKL inhibitors (announced by Catalyst Therapeutics Pty Ltd in November 2015, Targeting MLKL for the development of novel anti-inflammatory therapeutics, https://www.wehi.edu.au/sites/default/files/files/Catalyst_Therapeutics-Necroptosis-MLKL-2015_Nov.pdf).

In addition to NASH, AIH, and psoriasis, studies have been published showing that MLKL or necroptosis is involved in various diseases.

Experiments using a neonatal rat model with hypoxic-ischemic encephalopathy confirmed the effect of MLKL attenuating damage during brain development caused by hypoxic-ischemic encephalopathy (Qu Y et al., MLKL inhibition attenuates hypoxia-ischemia induced neuronal damage in developing brain, Exp Neurol, 2016) and MLKL therapeutics inhibited necroptosis associated with MLKL signaling in a stroke mouse model, by which alleviated ischemic leukoaraiosis and restored long-term neurological function (Chen Y et al., Necrostatin-1 Improves Long-term Functional Recovery Through Protecting Oligodendrocyte Precursor Cells After Transient Focal Cerebral ischemia in Mice. Neuroscience. 2018).

In addition, through experiments using ovalian cancer cells (in vitro) and ovalian cancer mouse models (in vivo), the effect of MLKL to inhibit metastasis of ovalian cancer by systemic CNL (cramide nanoliposome) was confirmed (Zhang X et al., Ceramide Nanoliposomes as a MLKL-Dependent, Necroptosis-Inducing, Chemotherapeutic Reagent in Ovarian Cancer, Mol Cancer Ther. 2018).

Overexpression of miR-500a-3P reduced phosphorylated MLKL and decreased RIP1 and RIPK3, confirming the possibility of treating acute renal injury through inhibition of MLKL singaling (Jiang L et al., hsa-miR-500a-3P alleviates kidney injury by targeting MLKL-mediated necroptosis in renal epithelial cells, FASEB J. 2019).

Additionally, experiments using the MLKL-KO mouse model confirmed that MLKL, independently of RIP3, plays an important role in NAFLD-related hepatic fat accumulation and inflammation. (Jun et al., Inhibition of mixed lineage kinase domain like pseudokinase decreases fat de novo synthesis and chemokine ligand expression in non-alcholic fatty liver disease, Journal of Gastroenterology and Hepatology, 2019).

Recently, it has been reported that MLKL forms a complex with RBM6 to regulate the mRNA stability and protein expression of adhesion molecules (ICAM1, VCAM1, and E-selectin), and MLKL inhibition reduces immune cell infiltration of acute inflammation (Dai et al., A necroptotic-independent function of MLKL in regulating endothelial cell adhesion molecule expression, Cell Death & Disease, 2020). In addition, it has been reported that MLKL is not only involved in necrotic core formation, but also involved in cellular lipid metabolism in atherosclerosis by regulating lipid droplet accumulation through endosomal trafficking. Through this, it was confirmed that MLKL plays an important role in antiatherogenic function and can play various roles in metabolic diseases (Rasheed et al., Loss of MLKL (Mixed Lineage Kinase Domain-Like Protein) Decreases Necrotic Core but Increases Macrophage Lipid Accumulation in Atherosclerosis, Arterioscler Thromb Vasc Biol, 2020).

On the other hand, existing targeted therapies can treat diseases by inhibiting sub-signaling functions via strongly binding to target proteins, but have the disadvantage of requiring continuous administration of high-concentration drugs to exert efficacy. This causes resistance and, as a result, many cases in which it cannot be used as a therapeutic agent occur. In addition, some target proteins are undruggable targets, and most of these undruggable targets do not have a binding pocket that can bind well to the target, so there are many difficulties in developing inhibitors. To overcome these problems, protein degrader (PROTAC or degrader) technology is rapidly emerging as a new alternative (CP Tinworth et al., Small molecule-mediated protein knockdown as a new approach to drug discovery, MedChemComm, 2016).

### DISCLOSURE

### Technical Problem

Therefore, the object to be achieved by the present invention is to provide compounds having MLKL-binding, inhibiting and/or degrading activities, pharmaceutical compositions containing the compound as an active ingredient, and medical uses for treating or preventing MLKL-related diseases.

Another object to be achieved by the present invention is to provide a method of treating or improving MLKL-related diseases, characterized by inhibiting MLKL activity, comprising administering the compound according to the present invention to a patient in need of treatment, improvement or prevention of MLKL-related diseases.

### Technical Solution

### SUMMARY

In order to achieve the above object, one embodiment of the present invention provides a compound represented by the following Chemical Formula 1 or a pharmaceutically acceptable salt thereof.

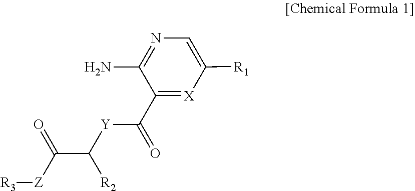

In the Chemical Formula 1
X is N or CH,
Y is S, O, NH or —N(C₁₋₆alkyl)-,
Z is a direct bond, —NH—, —N—(C₁₋₄alkyl)-, or

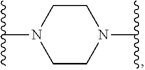 [SMILES: *N1CCN(*)CC1]

R₁ is C₁₋₁₀alkyl, aryl, heteroaryl, cycloalkyl, heterocycloalkyl, or -A-(B)-R₄, wherein R₁ is unsbstituted or substituted with at least one selected from C₁₋₆alkyl, halo-C₁₋₆alkyl, halogen, amino, alkoxy, nitro, cyano, —NRₐC(═O)R_{b}, —NRₐC(═O)NRₐR_{b}, —NRₐC(═O)OR_{b}, or —NRₐSO₂R_{b}, wherein Rₐ and R_{b} are the same or different and each independently hydrogen, C₁₋₆alkyl, aryl, heteroaryl, cycloalkyl, or heterocycloalkyl,
A is a 4-6-membered heteroaryl or heterocycloalkyl containing one or more N,
B is a direct bond (i.e. does not exist), or a 4-7-membered heterocycloalkyl containing one or more N,
R₄ is hydrogen, (lower)alkyl, halo-C₁₋₆alkyl, —C(═O)Rₐ, —C(═O)ORₐ, —PO₃Rₐ, —PO(ORₐ)(OR_{b}), —C(═O)NRₐR_{b}, —NRₐSO₂R_{b}, —SO₂Rₐ, —S(O)Rₐ, or —SO(N)Rₐ, wherein Rₐ and R_{b} are the same or different and each independently C₁₋₆alkyl, aryl, heteroaryl, cycloalkyl, or heterocycloalkyl,
R₂ is hydrogen, C₁₋₆alkyl, C₁₋₆alkoxy, halo-C₁₋₆alkyl, aryl, heteroaryl, or cycloalkyl, wherein the alkyl, alkoxy, aryl, heteroaryl or cycloalkyl is unsbstituted or substituted with at least one selected from C₁₋₆alkyl, halo-C₁₋₆alkyl, halogen, amino, alkoxy, nitro, cyano, —NRₐC(═O)R_{b}, —NRₐC(═O)NRₐR_{b}, —NRₐC(═O)OR_{b}, or —NRₐSO₂R_{b}, wherein Rₐ and R_{b} are the same or different and each independently hydrogen, halogen, amino, C₁₋₆alkyl, halo-C₁₋₆alkyl, aryl, heteroaryl, cycloalkyl or heterocycloalkyl, and
R₃ is C₁₋₁₀alkyl, aryl, heteroaryl, cycloalkyl, or heterocycloalkyl, wherein the aryl, heteroaryl, cycloalkyl, or heterocycloalkyl is unsbstituted or substituted with at least one selected from C₁₋₆alkyl, halogen, halo-C₁₋₆alkyl, C₁₋₆alkoxy, CN, —C(═O)Rₐ, —C(═O)ORₐ, —C(O)NRₐR_{b}, —SO₂Rₐ, —S(O)Rₐ, —SO(N)Rₐ, NO₂, —N(Rₐ)(R_{b}), aryl, aryl substituted with methyl, heterocycloalkyl, or heterocycloalkyl substituted with methyl, wherein Rₐ and R_{b} are the same or different and each independently hydrogen, halogen, amino, C₁₋₆alkyl, halo-C₁₋₆alkyl, aryl, heteroaryl, cycloalkyl or heterocycloalkyl.

In order to achieve the above object to be solved, another embodiment of the present invention provides a compound of Chemical Formula 2 or a pharmaceutically acceptable salt thereof.

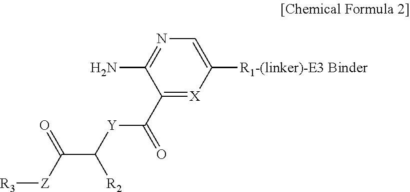

In the Chemical Formula 2,
X, Y, Z, R₂ and R₃ are the same as X, Y, Z, R₂ and R₃ of Chemical Formula 1 above,
R₁ is —(CH₂)ₙ—, aryl, heteroaryl, cycloalkyl, heterocycloalkyl, or -A-(B)-, wherein n is an integer of from 1 to 10,
A is a 4-6-membered heteroaryl or heterocycloalkyl containing one or more N,
B is a direct bond (i.e. does not exist), or a 4-7-membered heterocycloalkyl containing one or more N,
the linker may not exist, and if it exists, it is a connecting group that connects R₁ and E3 Binder, and
E3 Binder is an E3 ligase ligand.

Another embodiment of the present invention provides a pharmaceutical composition comprising a compound of Chemical Formula 1 or 2 or a pharmaceutically acceptable salt thereof, and a pharmaceutically acceptable carrier or additive.

Another embodiment of the present invention also provides a method for treating an MLKL-associated disease comprising administering to a subject a therapeutically effective amount of a compound of Chemical Formula 1 or 2 or a pharmaceutically acceptable salt thereof, wherein the MLKL-associated disease includes, but is not limited to, traumatic brain injury, stroke, bone marrow failure, (acute) pancreatitis, atherosclerosis, metabolic disease, ischemia-reperfusion injury, transplantation, infection, chronic obstructive pulmonary disease, remote lung injury, hepatotoxicity, (alcoholic and non-alcoholic) steatohepatitis, remote liver injury, autoimmune hepatitis, psoriasis, hypoxic ischemic encephalopathy, ovarian cancer, renal injury, fatty liver, inflammation, Crohn's colitis, ulcerative colitis, or ileitis. That is, the present invention provides medical uses of the compound of Chemical Formula 1 or 2 or a pharmaceutically acceptable salt thereof for treating or preventing the above disease.

The compounds, pharmaceutical compositions comprising such compounds, and their medical uses are more fully described in the detailed description that follows.

### DETAILED DESCRIPTION

The following description is illustrative only and is not intended to limit the invention, application, or use.

### Definition

The following terms used herein are defined as follows:

As used herein, the terms “substituent”, “radical”, “group”, “moiety”, and “fragment” may be used interchangeably.

As used herein, the term “patient” refers to an animal (e.g. cow, horse, sheep, pig, chicken, turkey, quail, cat, dog, mouse, rat, rabbit or guinea pig), preferably mammals such as non-primates and primates (e.g. monkeys and humans), most preferably humans.

As used herein, the term “alkyl” means a saturated straight chain or branched non-cyclic hydrocarbon, unless the context clearly dictates otherwise, having from 1 to 10 carbon atoms. “Lower alkyl” means alkyl having from 1 to 4 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl, while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein, if the term “C₁₋₆”, “C₁-C₆”, “C₁₋₆”, or “C₁-C₆” is used, it means the number of carbon atoms is from 1 to 6. For example, C₁₋₆ alkyl means an alkyl which carbon number is any integer of from 1 to 6.

As used herein, the terms “halogen” and “halo” mean fluorine, chlorine, bromine or iodine. In a preferred embodiment of the present invention, the halogen is chlorine or fluorine.

As used herein, the term “haloalkyl”, “haloalkoxy”, “haloalkenyl”, or “haloalkynyl” mean an alkyl, alkoxy, alkenyl or alkynyl group, wherein one or more hydrogen atoms are substituted with halogen atoms. For example, the haloalkyl includes —CF₃, —CHF₂, —CH₂F, —CBr₃, —CHBr₂, —CH₂Br, —CC1₃, —CHC1₂, —CH₂Cl, —Cl₃, —CHI₂, —CH₂I, —CH₂—CF₃, —CH₂—CHF₂, —CH₂—CH₂F, —CH₂—CBr₃, —CH₂—CHBr₂, —CH₂—CH₂Br, —CH₂—CC1₃, —CH₂—CHC1₂, —CH₂—CH₂Cl, —CH₂—Cl₃, —CH₂—CHI₂, —CH₂—CH₂I, and the like, wherein alkyl and halogen are as described above. In an embodiment of the present invention, haloalkyl is —CF₃.

As used herein, the term “alkanoyl” or “acyl” means a —C(O)alkyl group including —C(O)CH₃, —C(O)CH₂CH₃, —C(O)(CH₂)₂CH₃, —C(O)(CH₂)₃CH₃, —C(O)(CH₂)₄CH₃, —C(O)(CH₂)₅CH₃, and the like, wherein the alkyl is as defined above.

As used herein, the term “alkanoyloxy” or “acyloxy” means a —OC(O)alkyl group including —OC(O)CH₃, —OC(O)CH₂CH₃, —OC(O)(CH₂)₂CH₃, —OC(O)(CH₂)₃CH₃, —OC(O)(CH₂)₄CH₃, —OC(O)(CH₂)₅CH₃, and the like, wherein the alkyl is as defined above.

As used herein, the term “alkoxy” means —O-(alkyl) including —OCH₃, —OCH₂CH₃, —O(CH₂)₂CH₃, —O(CH₂)₃CH₃, —O(CH₂)₄CH₃, —O(CH₂)₅CH₃, and the like, wherein alkyl is as defined above.

As used herein, the term “lower alkoxy” means —O-(lower alkyl), wherein lower alkyl is as defined above.

As used herein, the term “aryl” means a carbocyclic aromatic group containing from 5 to 10 ring atoms. Representative examples include, but are not limited to, phenyl, tolyl, xylyl, naphthyl, tetrahydronaphthyl, anthracenyl, fluorenyl, indenyl, and azulenyl. A carbocyclic aromatic group can be unsubstituted or optionally substituted.

The term “aryloxy” is RO—, where R is aryl as defined above. “Arylthio” is RS—, where R is aryl as defined above.

As used herein, the term “cycloalkyl” means a monocyclic or polycyclic saturated ring having carbon and hydrogen atoms and having no carbon-carbon multiple bonds. Examples of monocyclic rings include, but are not limited to, (C₃-C₇)cycloalkyl groups, including cyclopropyl, cyclobutyl, cyclopentyl, cyclohexyl, and cycloheptyl. Examples of polycyclic rings include, but are not limited to, fused bicyclic rings such as octahydropentalene and decahydronaphthalene; spiro rings such as spiro[3.3]heptane, spiro[3.4]octane, spiro[3.5]nonane, spiro[4.4]nonane, spiro[4.5]decane, and spiro[5.5]undecane; and bridged bicycle rings such as bicyclo[2.1.1]hexane, bicyclo[2.2.1]heptane, and bicyclo[2.2.2]octane. A cycloalkyl group can be unsubstituted or optionally substituted. In an embodiment of the present invention, the cycloalkyl group is monocyclic ring.

As used herein, the term “mono-alkylamino” refers to —NH(alkyl) including —NHCH₃, —NHCH₂CH₃, —NH(CH₂)₂CH₃, —NH(CH₂)₃CH₃, —NH(CH₂)₄CH₃, —NH(CH₂)₅CH₃, and the like, wherein the alkyl is as defined above.

As used herein, the term “di-alkylamino”-N(alkyl)(alkyl) —N(CH₃)₂, —N(CH₂CH₃)₂, —N((CH₂)₂CH₃)₂, —N(CH₃)(CH₂CH₃), and the like, wherein each alkyl is each independently the alkyl as defined above.

As used herein, the term “alkylamino” includes mono-alkylamino and di-alkylamino as defined above.

As used herein, the terms “carboxyl” and “carboxy” mean —COOH.

As used herein, the term “aminoalkyl” refers to -(alkyl)-NH₂ including —CH₂—NH₂, —(CH₂)₂—NH₂, —(CH₂)₃—NH₂, —(CH₂)₄—NH₂, —(CH₂)₅—NH₂, and the like, wherein the alkyl is as defined above.

As used herein, the term “mono-alkylaminoalkyl” means -(alkyl)-NH(alkyl) including —CH₂—NH—CH₃, —CH₂—NHCH₂CH₃, —CH₂—NH(CH₂)₂CH₃, —CH₂—NH(CH₂)₃CH₃, —CH₂—NH(CH₂)₄CH₃, —CH₂—NH(CH₂)₅CH₃, —(CH₂)₂—NH—CH₃, and the like, wherein each alkyl is each independently is as defined above.

As used herein, the term “heteroaryl” means an aromatic heterocycle ring of 5- to 10-members and having at least one heteroatom selected from nitrogen, oxygen and sulfur, and containing at least 1 carbon atom, including both mono- and bicyclic ring systems. Representative heteroaryls are furan, 4H-pyran, pyrrole, imidazole, pyrazole, triazole, tetrazole, pyridine, pyrimidine, pyridazine, pyrazine, triazine, thiophene, ozaxole, isoxazole, thiazole, isothiazole, oxadiazole, 1H-azepine, benzofuran, benzothiophene, quinoline, indole, benzoxazole, benzimidazole, benzothiazole, cinnoline, phthalazine, quinazoline, purine, pyrazolopyridine, pyrazolopyrimidine, imidazopyridine, benzotriazole, indazole, triazopyridine and the like.

The term “heterocycle” or “heterocycloalkyl” means a 5- to 7-membered monocyclic, or 7- to 12-membered bicyclic, saturated heterocyclic ring which contains from 1 to 4 heteroatoms independently selected from nitrogen, oxygen and sulfur, and wherein the nitrogen and sulfur heteroatoms can be optionally oxidized, and the nitrogen heteroatom can be optionally quatemized. Representative heterocycles include oxiran, oxetan, tetrahydrofuran, tetrahydropyran, 1,4-dioxane, aziridine, azetidine, pyrrolidine, piperidine, piperazine, pyrrolidinone, hydantoine, valerolactam, thiirane, thietane, tetrahydrothiophene, tetrahydrothiopyra, morpholine, tetrahydropyridine, and tetrahydropyrimidine. Heterocycles include a bicyclic ring in which part of the heterocycle is fused to a benzene or cyclopenta-1,3-diene ring. The heterocycle can be attached via any heteroatom or carbon atom. In addition, heterocycles include fused bicyclic rings, spiro rings and bridged bicyclic rings in which one or more carbon atoms of the aforementioned polycyclic rings are replaced with nitrogen, oxygen or sulfur atoms. For example, when the heteroatom is nitrogen, these include, but are limited to, fused heterobicyclic rings such as octahydrocyclopenta[c]pyrrole, octahydropyrrolo[3,4-c]pyrrole, decahydroisoquinoline, and decahydro-2,6-naphthyridine; spiro (hetero)rings such as 2-azaspiro[3.3]heptane, 2,6-diazaspiro[3.3]heptane, 2-azaspiro[3.4]octane, 2,6-diazaspiro[3.4]octane, 2-azaspiro[3.5]nonane, 2,7-diazaspiro[3.5]nonane, 2-azaspiro[4.4]nonane, 2,7-diazaspiro[4.4]nonane, 8-azaspiro[4.5]decane, 2,8-diazaspiro[4.5]decane, 3-azaspiro[5.5]undecane, and 3,9-diazaspiro[5.5]undecane; and bridged heterobicyclic rings such as 2-azabicyclo[2.1.1]hexane, 2-azabicyclo[2.2.1]heptane, 2,5-diazabicyclo[2.2.1]heptane, 2-azabicyclo[2.2.2]octane, and 2,5-diazabicyclo[2.2.2]octane.

“Heterocycle fused to a phenyl” means a heterocycle attached to two adjacent carbon atoms of a phenyl ring, wherein the heterocycle is as defined above.

As used herein, the term “hydroxyalkyl” means an alkyl in which one or more hydrogen atoms are replaced by hydroxy, including —CH₂OH, —CH₂CH₂OH, —(CH₂)₂CH₂OH, —(CH₂)₃CH₂OH, —(CH₂)₄CH₂OH, —(CH₂)₅CH₂OH, —CH(OH)—CH₃, —CH₂CH(OH)CH₃, and the like, wherein the alkyl is as defined above.

As used herein, the term “sulfonyl” means —SO₃H.

As used herein, the term “sulfonylalkyl” means —SO₂-(alkyl) including —SO₂—CH₃, —SO₂—CH₂CH₃, —SO₂—(CH₂)₂CH₃, —SO₂—(CH₂)₃CH₃, —SO₂—(CH₂)₄CH₃, —SO₂—(CH₂)₅CH₃, and the like, wherein the alkyl is as defined above.

As used herein, the term “sulfinylalkyl” means —SO-(alkyl) including —SO—CH₃, —SO—CH₂CH₃, —SO—(CH₂)₂CH₃, —SO—(CH₂)₃CH₃, —SO—(CH₂)₄CH₃, —SO—(CH₂)₅CH₃, and the like, wherein the alkyl is as defined above.

“Thioalkyl” includes —S—CH₃, —S—CH₂CH₃, —S—(CH₂)₂CH₃, —S—(CH₂)₃CH₃, —S—(CH₂)₄CH₃, —S—(CH₂)₅CH₃, and the like, wherein the alkyl is as defined above.

As used herein, the term “substituted” means that the hydrogen atom of the moiety (e.g., alkyl, aryl, heteroaryl, heterocycle or cycloalkyl) being substituted is replaced with a substituent. In one embodiment, each carbon atom of a group being substituted is unsubstituted by more than two substituents. In another embodiment, each carbon atom of a group being substituted is unsubstituted by more than one substituent. In the case of a keto substituent, two hydrogen atoms are replaced with oxygen attached to the carbon by a double bond. Unless otherwise specified with respect to the substituent, the substituent of the present invention includes halogen, hydroxyl, (lower)alkyl, haloalkyl, mono- or di-alkylamino, aryl, heterocycle, —NO₂, —NRₐR_{b}, —NRₐC(═O)R_{b}, —NRₐC(═O)NRₐR_{b}, —NRₐC(═O)OR_{b}, —NRₐSO₂R_{b}, —ORₐ, —CN, —C(═O)Rₐ, —C(═O)ORₐ, —C(═O)NRₐR_{b}, —OC(═O)Rₐ, —OC(═O)ORₐ, —OC(═O)NRₐR_{b}, —NRₐSO₂R_{b}, —PO₃Rₐ, —PO(ORₐ)(OR_{b}), —SO₂Rₐ, —S(O)Rₐ, —SO(N)Rₐ (e.g. sulfoximine), —(Rₐ)S═NR_{b} (e.g. sulfilimine) and —SRₐ, wherein Rₐ and R_{b} are the same or different and are each independently hydrogen, halogen, amino, alkyl, haloalkyl, aryl or heterocycle, or Rₐ and R_{b} can form a heterocycle together with the attached nitrogen atom. Here, Rₐ and R_{b} may be plural depending on the bonded atoms. Preferably, in one embodiment of the present invention, unless otherwise specified, the substituent of the present invention is halogen, hydroxyl, C₁₋₃alkyl, —ORₐ, —CN, di-alkylamino, aryl, or heterocycle, wherein Rₐ is hydrogen, halogen, amino, alkyl, haloalkyl, aryl or heterocycle.

As used herein, the term “pharmaceutically acceptable salt(s)” refers to a salt prepared from active compounds according to the present disclosure with relatively non-toxic acids or bases, depending on the particular substituents of those compounds. When the compounds have a relatively acidic group, base-added salts can be obtained by contacting the neutral compounds with a sufficient amount of the desired base and a pure or inert solvent. Suitable pharmaceutically acceptable base addition salts include, but are not limited to sodium, potassium, calcium, aluminum, organic amino, magnesium salts and the like. When the compounds have a relatively basic group, acid-added salts can be obtained by contacting the neutral compounds with a sufficient amount of the desired acid and pure or inert solvent. Suitable pharmaceutically acceptable acid addition salts include salts derived from non-toxic organic acids including, but are not limited to, acetic acid, propionic acid, isobutyl acid, oxalic acid, maleic acid, malonic acid, benzoic acid, succinic acid, suberic acid, fumaric acid, mandelic acid, phthalic acid, benzenesulfonic acid, p-tolylsulfonic acid, citric acid, tartaric acid, methanesulfonic acid, and the like, and non-toxic inorganic acids including, but are not limited to, hydrochloric acid, hydrobromic acid, nitric acid, carbonic acid, monohydrogencarbonic acid, phosphoric acid, monohydrogenphosphric acid, dihydrogenphosphoric acid, sulfuric acid, monohydrogensulfuric acid, hydrogen iodide, phosphorous acid and the like. Also it includes a salt of amino acid such as arginate or its analogues, and it also includes analogues of organic acid such as glucuronic or galacturonic acid. Some specific compounds of this disclosure have both basic and acidic functionality for the conversion of compounds with a basic or acidic portion (addition) salts.

As used herein, “effective amount” refers to an amount of a compound of the invention sufficient to slow or minimize the progression of a disease related to MLKL or to provide a therapeutic benefit in the treatment or management of a disease related to MLKL. “Effective amount” also refers to an amount sufficient to inhibit or reduce the activity of MLKL, either in vitro or in vivo.

As used herein, the term “treatment” may be any one or more of preventive treatment, palliative treatment, and/or restorative treatment.

As used herein, the phrase “compound(s) of this/the invention” includes any compound(s) of Chemical Formula 1 or 2, as well as clathrates, hydrates, solvates, or polymorphs thereof. And, even if the term “compound(s) of the invention” does not mention its pharmaceutically acceptable sat, the term includes salts thereof. In one embodiment, the compounds of this disclosure include stereo-chemically pure compounds, e.g., those substantially free (e.g., greater than 85% ee, greater than 90% ee, greater than 95% ee, greater than 97% ee, or greater than 99% ee) of other stereoisomers. That is, if the compounds of Chemical Formula 1 or 2 according to the present disclosure or salts thereof are tautomeric isomers and/or stereoisomers (e.g., geometrical isomers and conformational isomers), such isolated isomers and their mixtures also are included in the scope of this disclosure. If the compounds of the present disclosure or salts thereof have an asymmetric carbon in their structures, their active optical isomers and their racemic mixtures also are included in the scope of this disclosure.

As used herein, the term “polymorph” refers to solid crystalline forms of a compound of this disclosure or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “solvate” means a compound or its salt according to this disclosure that further includes a stoichiometric or non-stoichiometric amount of a solvent bound by non-covalent intermolecular forces. Preferred solvents are volatile, non-toxic, and acceptable for administration to humans in trace amounts.

As used herein, the term “hydrate” means a compound or its salt according to this disclosure that further includes a stoichiometric or non-stoichiometric amount of water bound by non-covalent intermolecular forces.

As used herein, the term “clathrate” means a compound or its salt in the form of a crystal lattice that contains spaces (e.g., channels) that have a guest molecule (e.g., a solvent or water) trapped within.

If any compound (prodrug) is divided in the body to produce the compound or salt thereof of the present invention, such compound is also included in the scope of the present invention. As used herein and unless otherwise indicated, the term “prodrug” refers to a compound being capable of providing an active compound, particularly a compound of the present invention through hydrolysis, oxidation and other reactions under biological conditions (ex vivo or in vivo). Examples of prodrugs include biohydrolyzable moiety-containing compounds that are biohydrolyzed to yield a compound of the present invention, and biohydrolyzable moieties include, but are not limited to, biohydrolyzable amides, biohydrolyzable esters, biohydrolyzable carbamates, biohydrolyzable carbonates, biohydrolyzable ureides, and biohydrolyzable phosphates. Preferably, the prodrug of the compound having a carboxyl functional group is a lower alkyl ester of a carboxylic acid. Carboxylic esters are commonly formed by esterifying a portion of a carboxylic acid present in a molecule. Prodrugs can be easily prepared based on various known literature.

As used herein, the term “purified” means that when isolated, the isolate is greater than 90% pure, in one embodiment greater than 95% pure, in another embodiment greater than 99% pure and in another embodiment greater than 99.9% pure.

The term “hydrido” refers to a single —H atom (H) and is used interchangeably with the symbol “H” or the term “hydrogen”.

If a substituent is described as “optionally substituted”, the substituent may be (1) unsubstituted or (2) substituted with one or more of the defined substituents. If the substitutable position is unsubstituted, the default substituent is hydrogen.

As used herein, the singular “a” and “an” may include the plural forms unless the context clearly dictates otherwise.

The term “pharmaceutically acceptable” means suitable for use as a pharmaceutical preparation, and generally considered safe for such use. The term also means that it has been officially approved by the governing body of a country for this use, or is listed in the Korean Pharmacopoeia or the United States Pharmacopoeia.

### Compound of the Present Invention

The present invention provides a compound having a structure of Chemical Formula 1 or a pharmaceutically acceptable salt thereof.

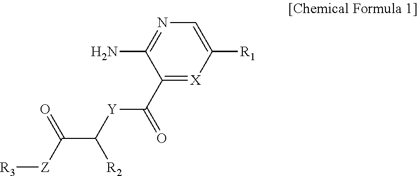

In the Chemical Formula 1,
X is N or CH,
Y is S, O, NH or —N(C₁₋₆alkyl)-,
Z is a direct bond, —NH—, —N—(C₁₋₄alkyl)-, or

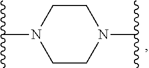 [SMILES: *N1CCN(*)CC1]

R₁ is C₁₋₁₀alkyl, aryl, heteroaryl, cycloalkyl, heterocycloalkyl, or -A-(B)-R₄, wherein R₁ is unsbstituted or substituted with at least one selected from C₁₋₆alkyl, halo-C₁₋₆alkyl, halogen, amino, alkoxy, nitro, cyano, —NRₐC(═O)R_{b}, —NRₐC(═O)NRₐR_{b}, —NRₐC(═O)OR_{b}, or —NRₐSO₂R_{b}, wherein Rₐ and R_{b} are the same or different and each independently hydrogen, C₁₋₆alkyl, aryl, heteroaryl, cycloalkyl, or heterocycloalkyl,
A is a 4-6-membered heteroaryl or heterocycloalkyl containing one or more N,
B is a direct bond (i.e. does not exist), or a 4-7-membered heterocycloalkyl containing one or more N,
R₄ is hydrogen, (lower)alkyl, halo-C₁₋₆alkyl, —C(═O)Rₐ, —C(═O)ORₐ, —PO₃Rₐ, —PO(ORₐ)(OR_{b}), —C(═O)NRₐR_{b}, —NRₐSO₂R_{b}, —SO₂Rₐ, —S(O)Rₐ, or —SO(N)Rₐ, wherein Rₐ and R_{b} are the same or different and each independently C₁₋₆alkyl, aryl, heteroaryl, cycloalkyl, or heterocycloalkyl,
R₂ is hydrogen, C₁₋₆alkyl, C₁₋₆alkoxy, halo-C₁₋₆alkyl, aryl, heteroaryl, or cycloalkyl, wherein the alkyl, alkoxy, aryl, heteroaryl or cycloalkyl is unsbstituted or substituted with at least one selected from C₁₋₆alkyl, halo-C₁₋₆alkyl, halogen, amino, alkoxy, nitro, cyano, —NRₐC(═O)R_{b}, —NRₐC(═O)NRₐR_{b}, —NRₐC(═O)OR_{b}, or —NRₐSO₂R_{b}, wherein Rₐ and R_{b} are the same or different and each independently hydrogen, halogen, amino, C₁₋₆alkyl, halo-C₁₋₆alkyl, aryl, heteroaryl, cycloalkyl or heterocycloalkyl, and
R₃ is C₁₋₁₀alkyl, aryl, heteroaryl, cycloalkyl, or heterocycloalkyl, wherein the aryl, heteroaryl, cycloalkyl, or heterocycloalkyl is unsbstituted or substituted with at least one selected from C₁₋₆alkyl, halogen, halo-C₁₋₆alkyl, C₁₋₆alkoxy, CN, —C(═O)Rₐ, —C(═O)ORₐ, —C(O)NRₐR_{b}, —SO₂Rₐ, —S(O)Rₐ, —SO(N)Rₐ, NO₂, —N(Rₐ)(R_{b}), aryl, aryl substituted with methyl, heterocycloalkyl, or heterocycloalkyl substituted with methyl, wherein Rₐ and R_{b} are the same or different and each independently hydrogen, halogen, amino, C₁₋₆alkyl, halo-C₁₋₆alkyl, aryl, heteroaryl, cycloalkyl or heterocycloalkyl.

Another embodiment of the present invention provides a compound represented by the Chemical Formula 1 or a pharmaceutically acceptable salt thereof, wherein
X is N or CH,
Y is S, O, NH or —N(C₁₋₆alkyl)-,
Z is a direct bond, —NH—, —N—(C₁₋₄alkyl)-, or

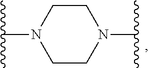 [SMILES: *N1CCN(*)CC1]

R₁ is aryl, heteroaryl, or -A-(B)-R₄, wherein A is a 4-6-membered heteroaryl or heterocycloalkyl containing one or more N; B is a direct bond, or a 4-7-membered heterocycloalkyl containing one or more N; R₄ is hydrogen, (lower)alkyl, halo-C₁₋₆alkyl, —C(═O)Rₐ, —C(═O)ORₐ, —PO₃Rₐ, —PO(ORₐ)(OR_{b}), —SO₂Rₐ, —S(O)Rₐ, or —SO(N)Rₐ, wherein Rₐ and R_{b} are the same or different and each independently C₁₋₆alkyl, cycloalkyl, or heterocycloalkyl,
R₂ is hydrogen, C₁₋₆alkyl, halo-C₁₋₆alkyl, aryl, heteroaryl, or cycloalkyl, wherein the alkyl, alkoxy, aryl, heteroaryl or cycloalkyl is unsbstituted or substituted with at least one selected from C₁₋₆alkyl, halo-C₁₋₆alkyl, aryl, halogen, C₁₋₆alkoxy, CN, or NO₂, and
R₃ is C₁₋₅alkyl, phenyl, pididyl, cyclohexyl, or piperidyl, wherein R₃ is unsbstituted or substituted with at least one selected from C₁₋₆alkyl, halogen, halo-C₁₋₆alkyl, C₁₋₆alkoxy, CN, NO₂, —N(C₁₋₃alkyl)(C₁₋₃alkyl), aryl, heterocycloalkyl, or heterocycloalkyl substituted with methyl.

Examples of the 4-6-membered heteroaryl containing one or more N as the heteroatom include pyrrole, pyrroline, pyrrolidine, imidazole, imidazoline, imidazolidine, pyrazole, pyrazoline, pyrazolidine, triazole, pyridine, piperidine, pyridazine, pyrimidine, pyrazine, piperazine, triazine and the like. Among them, imidazole, triazole, pyrazole, pyridine and the like are preferable for various purposes of the present invention. Pyrazole, triazole, pyridine and the like are more preferred for purposes of the present invention.

In the above paragraph, pyridine is meant to include tetrahydropyridine.

Examples of the 4-membered, 5-membered, 6-membered, or 7-membered (4-7-membered) heterocycloalkyl containing one or more N as the heteroatom include piperidine, piperazine, azetidine, pyrrolidine, and the like. Among them, piperidine, piperazine, pyrrolidine, azetidine and the like are preferred for various purposes of the present invention.

Preferably, yet another embodiment of the present invention provides a compound represented by the Chemical Formula 1 or a pharmaceutically acceptable salt thereof, wherein
X is N or CH,
Y is O, NH or —N(C₁₋₆alkyl)-,
Z is —NH—,
R₁ is -A-(B)-R₄, wherein A is imidazole, pyrazole, triazole, pyridine, piperidine, piperazine, or tetrahydropyridine; B is a direct bond, or piperidine, piperazine, azetidine, or pyrrolidine; R₄ is hydrogen, hydrogen, —C(O)—C₁₋₆alkyl, or —C(O)O—C₁₋₆alkyl,
R₂ is C₁₋₆alkyl, halo-C₁₋₆alkyl, or phenyl, wherein the alkyl or phenyl is unsbstituted or substituted with at least one selected from C₁₋₆alkyl, halo-C₁₋₆alkyl, halogen, C₁₋₆alkoxy, CN, or NO₂, and
R₃ is phenyl, pididyl, cyclohexyl, or piperidyl, wherein the phenyl, pididyl, cyclohexyl, or piperidyl is unsubstituted or substituted with at least one selected from C₁₋₆alkyl, halogen, or C₁₋₆alkoxy.

More preferably, yet another embodiment of the present invention provides a compound represented by the Chemical Formula 1 or a pharmaceutically acceptable salt thereof, wherein
X is N or CH (preferably, N),
Y is O or NH (preferably, O),
Z is —NH—,
R₁ is -A-(B)-R₄, wherein A is pyrazole, triazole, pyridine, or tetrahydropyridine; B is a direct bond, or piperidine, piperazine, azetidine, or pyrrolidine; R₄ is hydrogen, hydrogen, —C(O)—C₁₋₃alkyl, or —C(O)O—C₁₋₆alkyl,
R₂ is C₁₋₆alkyl, halo-C₁₋₆alkyl, or phenyl, wherein the alkyl or phenyl is unsbstituted or substituted with at least one selected from C₁₋₆alkyl, halo-C₁₋₆alkyl, halogen, or C₁₋₆alkoxy, and
R₃ is phenyl, pididyl, cyclohexyl, or piperidyl, wherein the phenyl, pididyl, cyclohexyl, or piperidyl is unsubstituted or substituted with at least one selected from C₁₋₆alkyl, halogen, or C₁₋₆alkoxy.

The present invention also provides a method for preparing a compound that inhibits or degrades MLKL using the compound represented by Chemical Formula 1 according to the present invention. In one embodiment of the present invention, the compound that inhibits or degrades MLKL may be a compound of Chemical Formula 2 or a salt thereof.

The present invention also provides a method for preparing a compound that inhibits or degrades MLKL, characterized in that the compound represented by Chemical Formula 1 according to the present invention is included in the entire structure. In one embodiment of the present invention, the compound that inhibits or degrades MLKL may be a compound of Chemical Formula 2 or a salt thereof.

One embodiment of the present invention also provides a compound of Chemical Formula 2 or a pharmaceutically acceptable salt thereof.

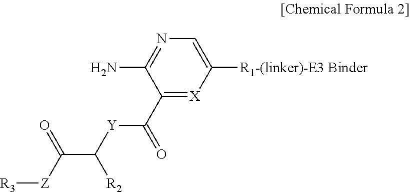

In the Chemical Formula 2,
X, Y, Z, R₂ and R₃ are the same as X, Y, Z, R₂ and R₃ of Chemical Formula 1 above,
R₁ is —(CH₂)ₙ—, aryl, heteroaryl, cycloalkyl, heterocycloalkyl, or -A-(B)-, wherein n is an integer of from 1 to 10,
A is a 4-6-membered heteroaryl or heterocycloalkyl containing one or more N,
B is a direct bond (i.e. does not exist), or a 4-7-membered heterocycloalkyl containing one or more N,
the linker may not exist, and if it exists, it is a connecting group that connects R₁ and E3 Binder, and
E3 Binder is an E3 ligase ligand.

Another embodiment of the present invention provides a compound represented by the Chemical Formula 2 or a pharmaceutically acceptable salt thereof, wherein
X is N or CH,
Y is S, O, NH or —N(C₁₋₆alkyl)-,
Z is a direct bond, —NH—, —N—(C₁₋₄alkyl)-, or

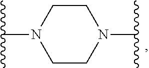 [SMILES: *N1CCN(*)CC1]

R₂ is hydrogen, C₁₋₆alkyl, halo-C₁₋₆alkyl, aryl, heteroaryl, or cycloalkyl, wherein the alkyl, alkoxy, aryl, heteroaryl or cycloalkyl is unsbstituted or substituted with at least one selected from C₁₋₆alkyl, halo-C₁₋₆alkyl, aryl, halogen, C₁₋₆alkoxy, CN, or NO₂,
R₃ is C₁₋₅alkyl, phenyl, pididyl, cyclohexyl, or piperidyl, wherein R₃ is unsbstituted or substituted with at least one selected from C₁₋₆alkyl, halogen, halo-C₁₋₆alkyl, C₁₋₆alkoxy, CN, NO₂, or aryl, and
R₁ is aryl, heteroaryl, or -A-(B)-, wherein A is a 4-6-membered heteroaryl or heterocycloalkyl containing one or more N; and B is a direct bond, or a 4-7-membered heterocycloalkyl containing one or more N.

Examples of the 4-6-membered heteroaryl containing one or more N as the heteroatom include pyrrole, pyrroline, pyrrolidine, imidazole, imidazoline, imidazolidine, pyrazole, pyrazoline, pyrazolidine, triazole, pyridine, piperidine, pyridazine, pyrimidine, pyrazine, piperazine, triazine and the like. Among them, imidazole, triazole, pyrazole, pyridine and the like are preferable for various purposes of the present invention. Pyrazole, triazole, pyridine and the like are more preferred for purposes of the present invention.

In the above paragraph, pyridine is meant to include tetrahydropyridine.

Examples of the 4-7-membered heterocycloalkyl containing one or more N as the heteroatom include piperidine, piperazine, azetidine, pyrrolidine, and the like. Among them, piperidine, piperazine, pyrrolidine, azetidine and the like are preferred for various purposes of the present invention.

Preferably, yet another embodiment of the present invention provides a compound represented by the Chemical Formula 2 or a pharmaceutically acceptable salt thereof, wherein
X is N or CH,
Y is O, NH or —N(C₁₋₆alkyl)-,
Z is —NH—,
R₂ is C₁₋₆alkyl, halo-C₁₋₆alkyl, or phenyl, wherein the alkyl or phenyl is unsbstituted or substituted with at least one selected from C₁₋₆alkyl, halo-C₁₋₆alkyl, halogen, C₁₋₆alkoxy, CN, or NO₂,
R₃ is phenyl, pididyl, cyclohexyl, or piperidyl, wherein the phenyl, pididyl, cyclohexyl, or piperidyl is unsbstituted or substituted with at least one selected from C₁₋₆alkyl, halogen, or C₁₋₆alkoxy, and
R₁ is -A-(B)-, wherein A is imidazole, pyrazole, triazole, pyridine, piperidine, piperazine, or tetrahydropyridine; B is a direct bond, or piperidine, piperazine, azetidine, or pyrrolidine.

More preferably, yet another embodiment of the present invention provides a compound represented by the Chemical Formula 2 or a pharmaceutically acceptable salt thereof, wherein
X is N or CH (preferably, N),
Y is O or NH (preferably, O),
Z is —NH—,
R₂ is C₁₋₆alkyl, halo-C₁₋₆alkyl, or phenyl, wherein the alkyl or phenyl is unsbstituted or substituted with at least one selected from C₁₋₆alkyl, halo-C₁₋₆alkyl, halogen, or C₁₋₆alkoxy,
R₃ is phenyl, pididyl, cyclohexyl, or piperidyl, wherein the phenyl, pididyl, cyclohexyl, or piperidyl is unsbstituted or substituted with at least one selected from C₁₋₆alkyl, halogen, or C₁₋₆alkoxy, and
R₁ is -A-(B)-, wherein A is imidazole, pyrazole, triazole, pyridine, or tetrahydropyridine; B is a direct bond, or piperidine, piperazine, azetidine, or pyrrolidine.

In one embodiment of the present invention, the linker of Chemical Formula 2 is a linker that connects the compound of Chemical Formula 1 according to the present invention and the E3 ligase ligand, and these linkers can be connected to the MLKL binder compound by an alkyl bond through aldehyde, chloride, bromide, iodide, or tosylate, by an amide bond through an acid, or by an amide bond through amine. Such linkers include, for example, those disclosed in prior patent publications US20180353501 A1, WO2019199816 A1, WO2019023553 A1, US20180125821 A1, US20190192668 A1, WO2017197056 A1, WO2019186358 A1, and/or WO2018089736 A1. The contents described in the prior patent publications are incorporated herein in their entirety by this reference.

In a typical embodiment, the linker has a chain of 2 to 14, 15, 16, 17, 18 or 20 or more carbon atoms of which one or more carbons can be replaced by a heteroatom such as O, N, S, or P. In certain embodiments, the chain has 2, 3, 4, 5, 6, 7, 8, 9, 10, 11, 12, 13, 14, 15, 16, 17, 18, 19 or 20 contiguous atoms in the chain. For example, the chain may include 1 or more ethylene glycol units that can be contiguous, partially contiguous or non-contiguous (for example, 2, 3, 4, 5, 6, 7, 8, 9, 10, 11 or 12 ethylene glycol units). In certain embodiments the chain has at least 1, 2, 3, 4, 5, 6, 7, or 8 contiguous chains which can have branches which can be independently alkyl, heteroalkyl, aryl, heteroaryl, alkenyl, or alkynyl, aliphatic, heteroaliphatic, cycloalkyl or heterocyclic substituents.

In other embodiments, the linker can include or be comprised of one or more of ethylene glycol, propylene glycol, lactic acid and/or glycolic acid. In general, propylene glycol adds hydrophobicity, while propylene glycol adds hydrophilicity. Lactic acid segments tend to have a longer half-life than glycolic acid segments. Block and random lactic acid-co-glycolic acid moieties, as well as ethylene glycol and propylene glycol, are known in the art to be pharmaceutically acceptable and can be modified or arranged to obtain the desired half-life and hydrophilicity. In certain aspects, these units can be flanked or interspersed with other moieties, such as aliphatic, including alkyl, heteroaliphatic, aryl, heteroaryl, heterocyclic, cycloalkyl, etc., as desired to achieve the appropriate drug properties.

In another embodiment of the present invention, the linker of Chemical Formula 2 may be a direct bond or have a structure of Chemical Formula 6 below.

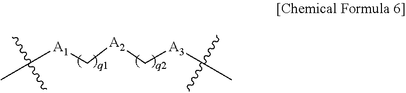

In the Chemical Formula 6,
A₁ is a direct bond, —C(O)—, —C(O)NH—, or —S(O)₂—,
A₂ is a direct bond, O, —C(O)—, —CH(OH)—, —N(C₁₋₃alkyl)-, —(OCH₂CH₂)_{q3}—, C₃₋₈cycloalkyl, heterocycle, or heteroaryl (preferably, imidazole, triazole, piperidine, piperazine, pyrrolidine, azetidine, or

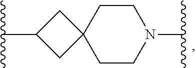 [SMILES: *C1CC2(CCN(*)CC2)C1]

more preferably cyclohexane, imidazole, or triazole),
A₃ is a direct bond, CO, NH, CC, heterocycloalkyl having bi-cyclic structure (preferably,

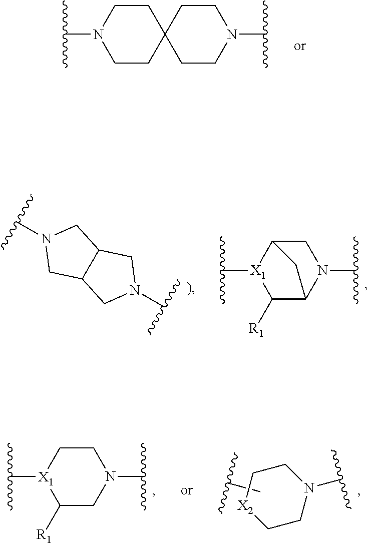

wherein, X₁ is N, CH or C(OH), X₂ is O or CH, and R₁ is hydrogen, —C₁₋₃alkyl, or —C₁₋₃alkyl-OH, and
q1, q2 and q3 are each independently an integer of from 0 to 12 (preferably, q3 is an integer of from 0 to 5).

In the present specification, “*”, “ [symbol] ”, or “L” means that it is connected to another moiety.

In the linker, * on the left means that it is linked to the MLKL binder moiety, and * on the right means that it is linked to the E3 ligase ligand compound.

Preferably, in another embodiment of the present invention, the linker of Chemical Formula 2 has any one of the following structures:

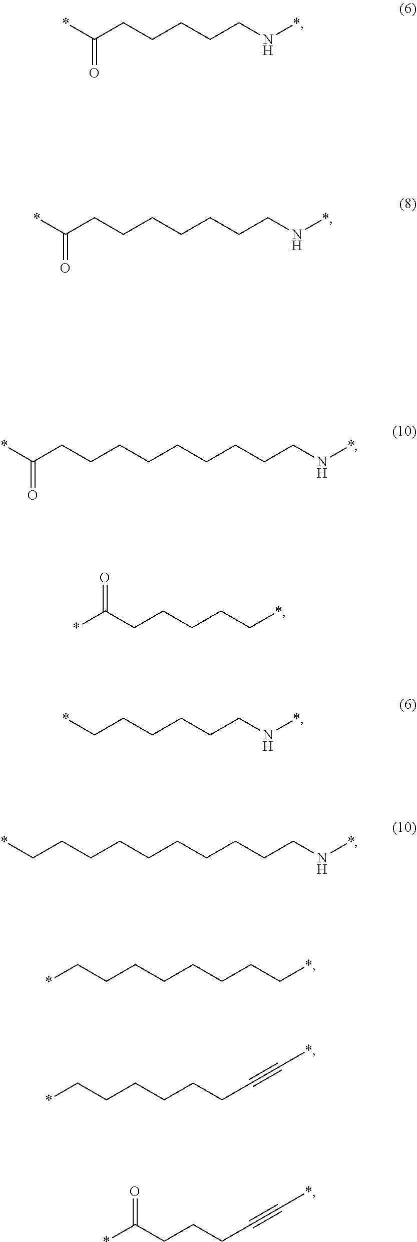

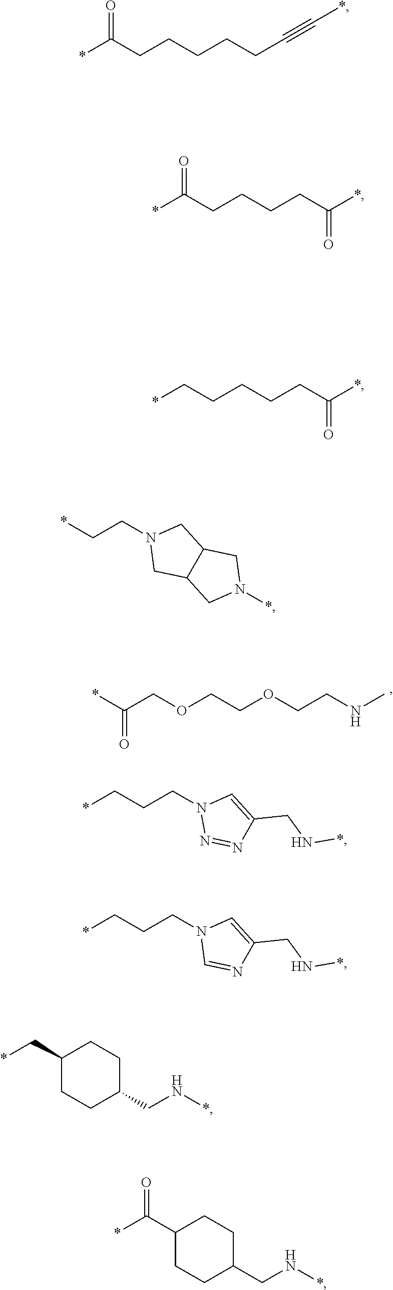

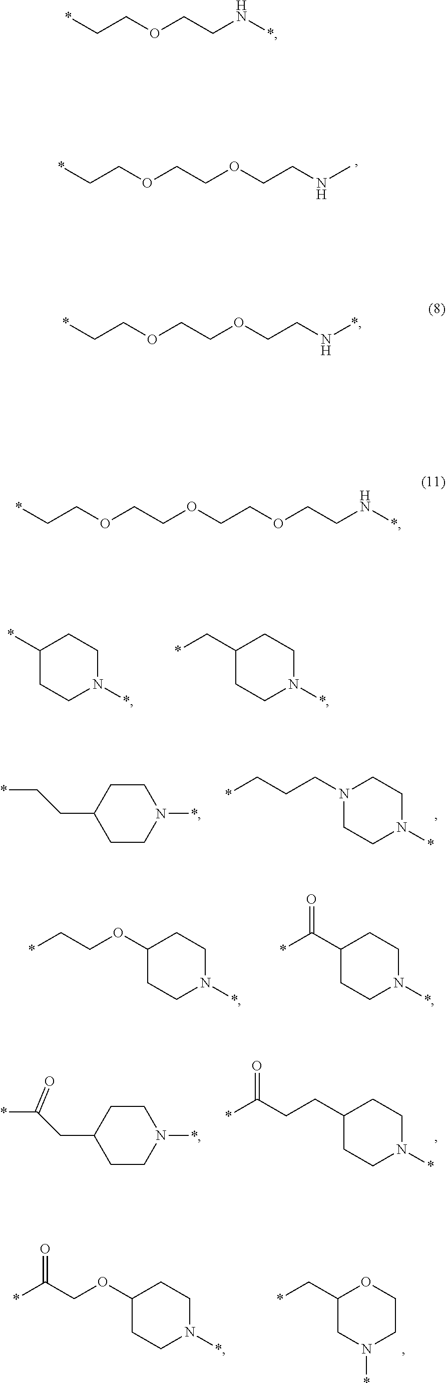

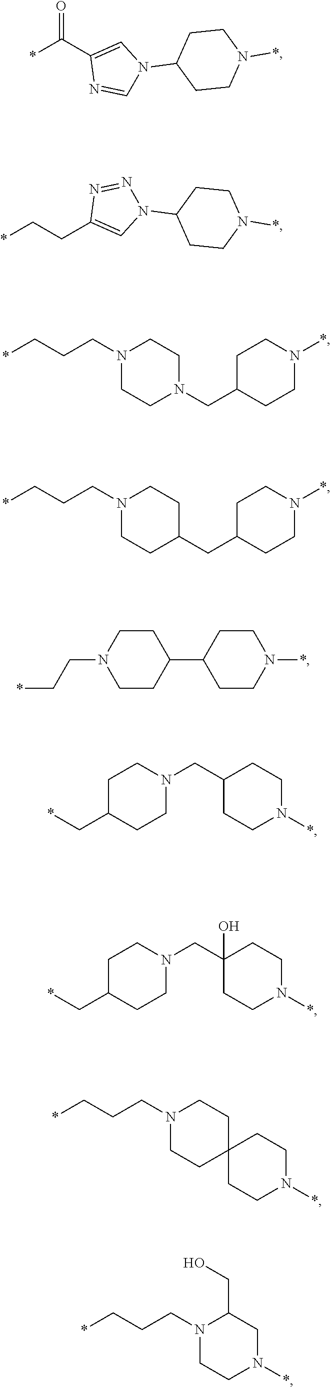

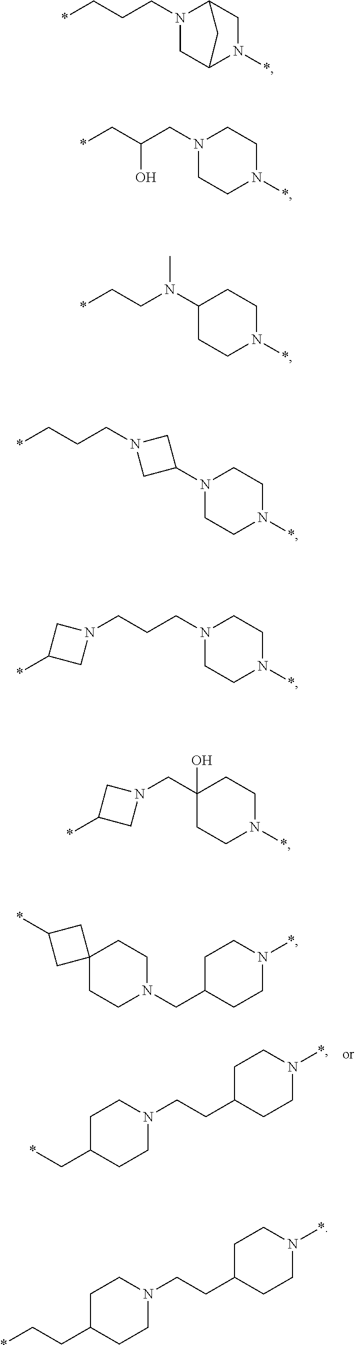

In a preferred embodiment of the present invention, the linker is a form containing at least one ring composed of 5 or more elements rather than a structure such as a straight-chain alkyl or PEG.

In one embodiment of the present invention, the E3 binder of Chemical Formula 2 refers to an E3 ligase ligand. After the MLKL binder moiety of Chemical Formula 1 according to the present invention binds to MLKL, the E3 binder links the target protein, which is MLKL, with E3 ligase (step 1); ubiquitin from E2 complexed with E3 ligase is poly-ubiquitinated on lysine of the target protein (step 2); and then MLKL is inhibited or degraded through the degradation process of the proteasome (step 3). These degraders can be recycled in the same way (step 4).

Examples of the E3 ligase binder compound include those disclosed, e.g., in M. Toure, C. M. Crews, Angew. Chem. Int. Ed. 2016, 55, 1966, T. Uehara et al. Nature Chemical Biology 2017, 13, 675, WO 2017/176708, US 2017/0281784, WO 2017/161119, WO 2017/176957, WO 2017/176958, WO 2015/160845, US 2015/0291562, WO 2016/197032, WO 2016/105518, US 2018/0009779, WO 2017/007612, 2018/0134684, WO 2013/106643, US 2014/0356322, WO 2002/020740, US 2002/0068063, WO 2012/078559, US 2014/0302523, WO 2012/003281, US 2013/0190340, US 2016/0022642, WO 2014/063061, US 2015/0274738, WO 2016/118666, US 2016/0214972, WO 2016/149668, US 2016/0272639, WO 2016/169989, US 2018/0118733, WO 2016/197114, US 2018/0147202, WO 2017/011371, US 2017/0008904, WO 2017/011590, US 2017/0037004, WO 2017/079267, US 2017/0121321, WO 2017/117473, WO 2017/117474, WO 2013/106646, WO 2014/108452, WO 2017/197036, WO 2017/197046, WO 2017/197051, WO 2017/197055, US 2019/0192668, US 2018/0155322, WO2021/053555 or WO2017-197056. The contents disclosed in the prior patent publications or papers are incorporated herein in their entirety by this reference. Such an E3 binder compound may be connected to a linker through, for example, an alkyl or amide bond through an amine, but the present invention is not limited to such a preparation method.

More preferably, as the E3 ligase binder compound according to the present invention, for example, compounds disclosed in US 2019/0192668 A1, US 2018-0155322 A1, or WO 2017-197056 A1 may be used. The contents disclosed in the prior patent publications or papers are incorporated herein in their entirety by this reference. Such an E3 binder compound may be connected to a linker by, for example, an alkyl or amide bond through an amine, but the present invention is not limited to such a preparation method.

Preferably, in one embodiment of the present invention, the E3 Binder is *-(Chemical Formula X)-Chemical Formula Y,
wherein Chemical Formula X does not exist or is a moiety connecting Chemical Formula Y and the rest of Chemical Formula 2, and

Chemical Formula Y is CRBN E3 Ubiquitin Ligase binding moiety, VHL E3 Ubiquitin Ligase binding moiety, or IAP E3 Ubiquitin Ligase binding moiety.

Preferably, in one embodiment of the present invention, Chemical Formula Y according to the present invention is CRBN_(cereblon) E3 Ubiquitin Ligase binding moiety, and for example, CRBN E3 Ubiquitin Ligase binding moiety disclosed in US 2019-0192668 may be used. The contents disclosed in the prior patent publications or papers are incorporated herein in their entirety by this reference.

Preferably, in one embodiment of the present invention, CRBN E3 Ubiquitin Ligase binding moiety according to the present invention, that is, Chemical Formula Y is Chemical Formula 3:

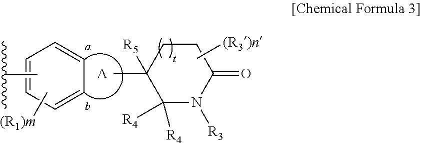

In the Chemical Formula 3,
the

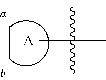

is any one of the following:

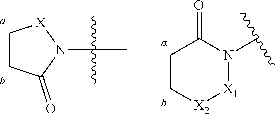

X is C(O) or C(R₃)₂;
X₁-X₂ is N═N, C(R₃)═N or C(R₃)₂—C(R₃)₂;
each R₁ is each independently halogen, nitro, NH₂, OH, C(O)OH, C₁-C₆ alkyl, or C₁-C₆ alkoxy;
each R₃ is each independently H or C₁-C₃ alkyl optionally substituted with C₆-C₁₀ aryl or 5- to 10-membered heteroaryl;
each R₃′ is each independently C₁-C₃ alkyl;
each R₄ is each independently H or C₁-C₃ alkyl; or two R₄, together with the carbon atom to which they are attached, form C(O), a C₃-C₆ cycloalkyl, or a 4-, 5-, or 6-membered heterocycle containing 1 or 2 heteroatoms selected from N and O;
R₅ is D (deuterium), H, C₁-C₃ alkyl, F, or Cl;
t is 0, 1 or 2;
m is 0, 1, 2 or 3; and
n and n′ are each independently 0, 1 or 2.

For example, as the Chemical Formula 3, moieties having the following structures may be used.

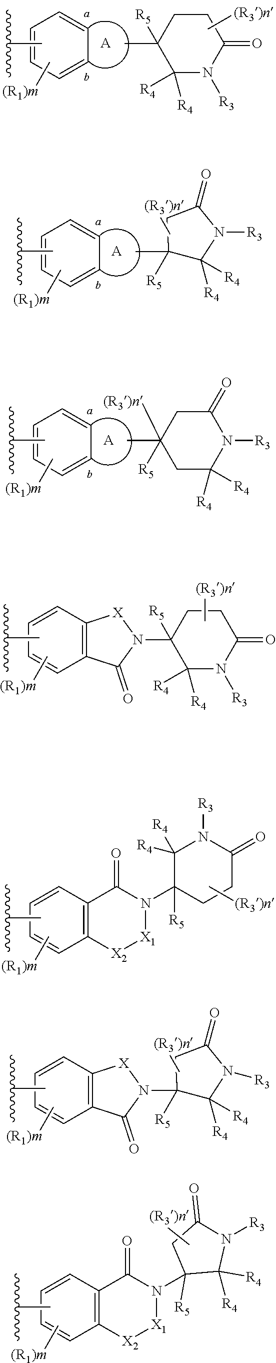

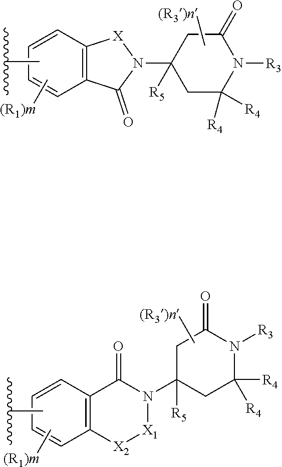

More preferably, in one embodiment of the present invention, Chemical Formula 3 has the following structure:

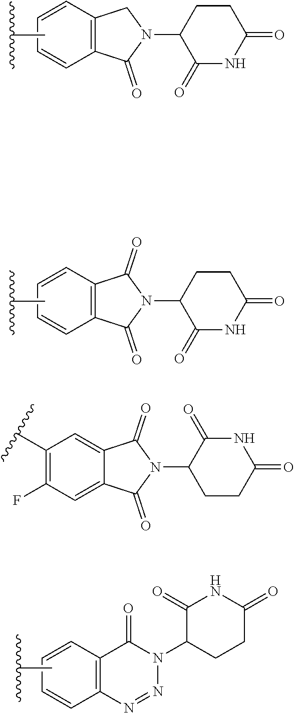

Preferably, in one embodiment of the present invention, Chemical Formula Y according to the present invention is VHL E3 Ubiquitin Ligase binding moiety, and for example, VHL E3 Ubiquitin Ligase binding moiety disclosed in US 2019-0192668 may be used. The contents disclosed in the prior patent publications or papers are incorporated herein in their entirety by this reference.

Preferably, in one embodiment of the present invention, VHL E3 Ubiquitin Ligase binding moiety according to the present invention, that is, Chemical Formula Y is Chemical Formula 4:

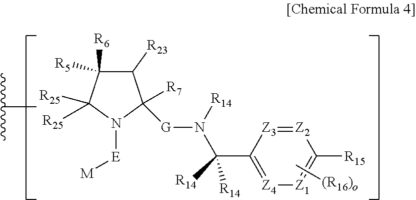

In the Chemical Formula 4,
each R₅ and R₆ is each independently OH, SH, or optionally substituted alkyl, or R₅, R₆, and the carbon atom to which they are attached form a carbonyl;
R₇ is H or optionally substituted alkyl;
E is a direct bond, C═O, or C═S;
G is a direct bond, optionally substituted alkyl, —COOH, or CO, or C═N—R₈, wherein R₈ is H, CN, optionally substituted alkyl, or optionally substituted alkoxy;
M is optionally substituted aryl, optionally substituted heteroaryl, optionally substituted heterocycle or

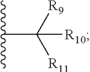

each R₉ and R₁₀ are each independently H, optionally substituted alkyl, optionally substituted cycloalkyl, optionally substituted hydroxyalkyl, optionally substituted thioalkyl, disulphide connected to linker or X, optionally substituted heteroaryl, or haloalkyl; or R₉, R₁₀, and the carbon atom to which they are attached form an optionally substituted cycloalkyl;
R₁₁ is optionally substituted heterocycle, optionally substituted alkoxy, optionally substituted heteroaryl, optionally substituted aryl, or

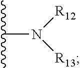

R₁₂ is H or optionally substituted alkyl;
R₁₃ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate,
each R₁₄ is each independently H, haloalkyl, optionally substituted cycloalkyl, optionally substituted alkyl or optionally substituted heterocycloalkyl;
R₁₅ is H, optionally substituted heteroaryl, haloalkyl, optionally substituted aryl, optionally substituted alkoxy, or optionally substituted heterocycle;
each R₁₆ is each independently halogen, optionally substituted alkyl, optionally substituted haloalkyl, CN, or optionally substituted haloalkoxy;
each R₂₅ is each independently H or optionally substituted alkyl; or both R₂₅ groups can be taken together to form an oxo or optionally substituted cycloalkyl group;
R₂₃ is H or OH;
Z₁, Z₂, Z₃, and Z₄ are each independently C or N; and
o is 0, 1, 2, 3, or 4.

Example of Chemical Formula 4 is a moiety having the following structure:

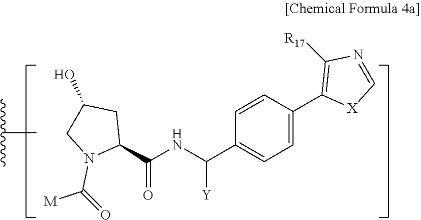

In the Chemical Formula 4a,
M is optionally substituted aryl, optionally substituted heteroaryl, optionally substituted heterocycle or

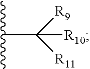

each R₉ and R₁₀ is each independently H; optionally substituted alkyl, optionally substituted cycloalkyl, optionally substituted hydroxyalkyl, optionally substituted thioalkyl, a disulphide connected to linker or X, optionally substituted heteroaryl, or haloalkyl; or R₉, R₁₀, and the carbon atom to which they are attached form an optionally substituted cycloalkyl;
R₁₁ is optionally substituted heterocycle, optionally substituted alkoxy, optionally substituted heteroaryl, optionally substituted aryl, or

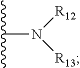

R₁₂ is H or optionally substituted alkyl;
R₁₃ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate;
X is O or S;
Y is H, methyl or ethyl; and
R₁₇ is H, methyl, ethyl, hydoxymethyl or cyclopropyl.

Preferably, in one embodiment of the present invention, Chemical Formula 4 has the following structure:

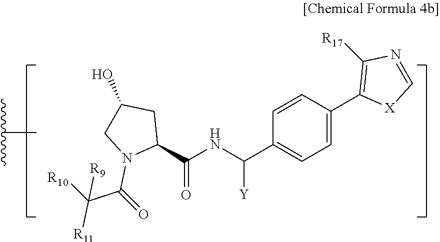

In the Chemical Formula 4b,
R₉, R₁₀, R₁₁, R₁₂, R₁₃, X, Y and R₁₇ are the same as mentioned in Chemical Formula 4a.

More preferably, in one embodiment of the present invention, Chemical Formula 4 has the following structure:

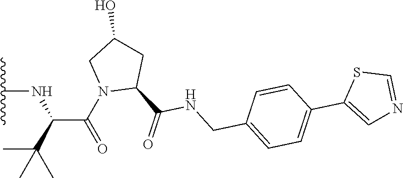

Preferably, in one embodiment of the present invention, Chemical Formula Y according to the present invention is CRBN (cereblon) E3 Ubiquitin Ligase binding moiety, and for example, CRBN E3 Ubiquitin Ligase binding moiety disclosed in WO 2019/186358 may be used. The contents disclosed in the prior patent publications or papers are incorporated herein in their entirety by this reference.

Preferably, in one embodiment of the present invention, CRBN E3 Ubiquitin Ligase binding moiety according to the present invention, that is, Chemical Formula Y is Chemical Formula 5:

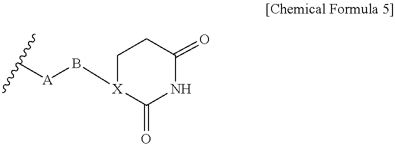

In the Chemical Formula 5,
X is CH or N,
A is C₁₋₃alkyl, aryl, heteroaryl, or heterocycloalkyl, wherein the aryl, heteroaryl, or heterocycloalkyl is unsubstituted or one or more hydrogen of the aryl, heteroaryl, or heterocycloalkyl is substituted with at least one selected from the group consisting of ═O, C₁₋₃alkoxy, C₁₋₃alkyl, halogen, nitrile, nitro, di-C₁₋₂alkylamine, —COOH, and —COO—C₁₋₂alkyl, and
B is a direct bond or —C(O)NH—.

In a preferred embodiment of the present invention, moieties having the following structure may be used as the Chemical Formula 5.

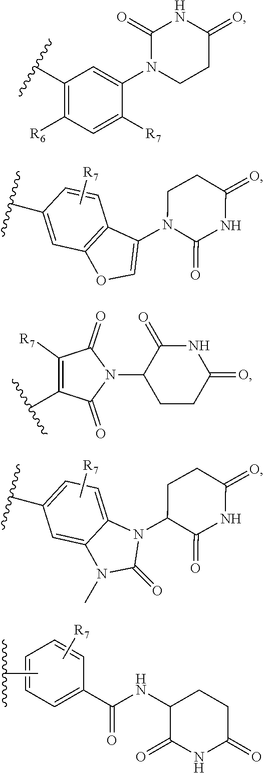

In the above structures, R₆ and R₇ are each independently hydrogen, halogen, —C₁₋₃alkyl, or —C₁₋₃alkoxy.

In a preferred embodiment of the present invention, moieties having the following structure may be used as the Chemical Formula 5.

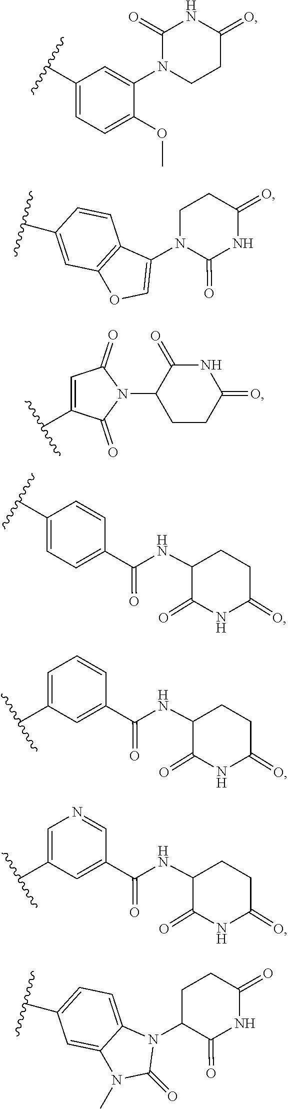

Preferably, in one embodiment of the present invention, Chemical Formula Y according to the present invention is IAP E3 Ubiquitin Ligase binding moiety, and for example, IAP E3 Ubiquitin Ligase binding moiety disclosed in WO 2018/118598 may be used. The contents disclosed in the prior patent publications or papers are incorporated herein in their entirety by this reference.

Preferably, in one embodiment of the present invention, IAP E3 Ubiquitin Ligase binding moiety according to the present invention, that is, Chemical Formula Y may have any structure of the following structures:

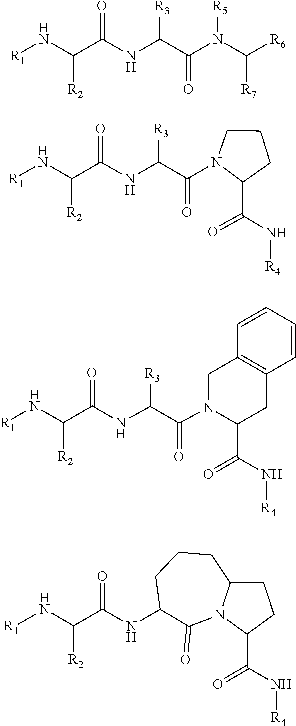

In the IAP E3 Ubiquitin Ligase binding moiety above,
R¹ is H or alkyl;
R² is H or alkyl;
R³ is H, alkyl, cycloalkyl and heterocycloalkyl;
R⁴ is alkyl, cycloalkyl, heterocycloalkyl, cycloalkylalkyl, heterocycloalkylalkyl, aryl, arylalkyl, heteroaryl, or heteroarylalkyl, wherein these are further optionally substituted with 1-3 substituents as described above;
R⁵ and R⁶ are each independently H, alkyl, cycloalkyl, or heterocycloalkyl, or more preferably, R⁵ and R⁶ taken together form a pyrrolidine or a piperidine ring further optionally fused to 1-2 cycloalkyl, heterocycloalkyl, aryl or heteroaryl rings, each of which can then be further fused to another cycloalkyl, heterocycloalkyl, aryl or heteroaryl ring; and
R⁷ is —C(O)NH—R₄.

Preferably, in one embodiment of the present invention, Chemical Formula X present in the E3 Binder structure (*-(Chemical Formula X)-Chemical Formula Y) is a direct bond or Chemical Formula 7:

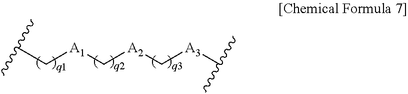

In the Chemical Formula 7,
A₁ is a direct bond, CO, or —C(O)NH—,
A₂ and A₃ are each independently a direct bond, O, CO, NH, CC, phenyl, heterocycloalkyl (preferably, piperidine, piperazine, pyrrolidine, or azetidine), and
q1, q2 and q3 are each independently an integer of from 0 to 3.

In a preferable embodiment of the present invention, Chemical Formula X present in the E3 Binder structure is a direct bond or any one of the following structures:

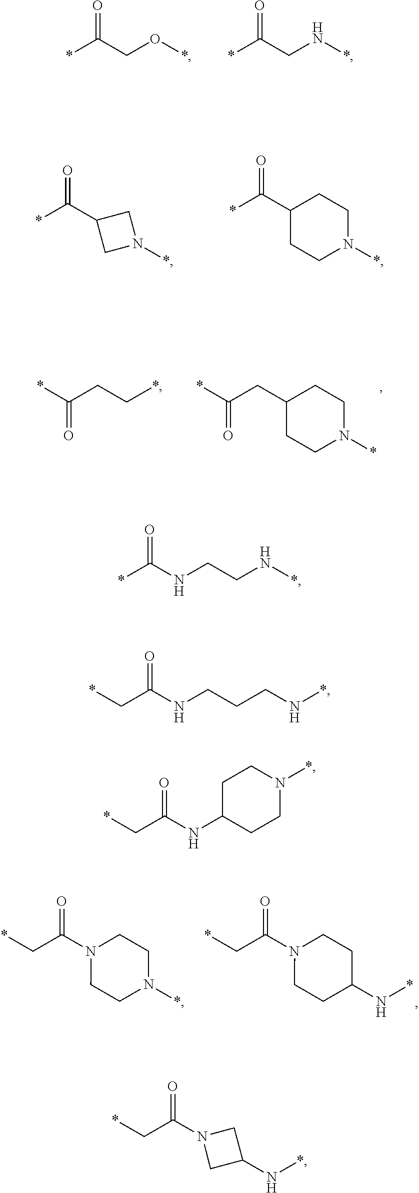

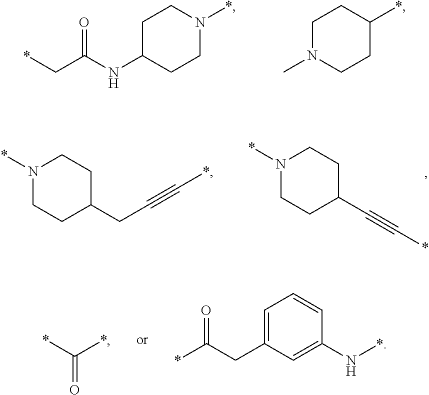

In order to achieve the above-mentioned object, the present inventors performed various evaluation experiments after synthesizing various compounds for the purpose of obtaining compounds and medical uses thereof, wherein the compounds have excellent MLKL inhibition, degradation and/or binding activity, high selectivity for them, and preferably effectively degrade MLKL, resulting in good treatment or prevention of MLKL-related diseases and reduced other side effects. Finally, the present invention was completed by confirming that the compounds of the present invention are suitable for the purpose of the present invention.

Non-limiting examples of compounds of Chemical Formula 1 according to the present invention include the compounds in Table 1 below and pharmaceutically acceptable salts thereof.

TABLE 1

| Compound | IUPAC Name |
|---|---|
| TDH-001 | (R)-2-((4-fluorophenyl)amino)-2-oxo-1-phenylethyl 3-aminopyrazine-2-carboxylate |
| TDH-002 | (S)-2-((4-fluorophenyl)amino)-2-oxo-1-phenylethyl 3-aminopyrazine-2-carboxylate |
| TDH-066 | (R)-1′-(tert-butyl) 5-(2-((4-fluorophenyl)amino)-2-oxo-1-phenylethyl) 6-amino-3′,6′-dihydro- |
| | [3,4′-bipyridine]-1′,5(2′H)-dicarboxylate |
| TDH-067 | (S)-3-amino-6-bromo-N-(2-((4-fluorophenyl)amino)-2-oxo-1-phenylethyl)pyrazine-2- |
| | carboxamide |
| TDH-068 | (R)-2-((4-fluorophenyl)amino)-2-oxo-1-phenylethyl 3-amino-6-(1,2,3,6-tetrahydropyridin-4- |
| | yl)pyrazine-2-carboxylate hydrochloride |
| TDH-069 | (R)-2-((4-fluorophenyl)amino)-2-oxo-1-phenylethyl 3-amino-6-(1-(piperidin-4-yl)-1H- |
| | pyrazol-4-yl)pyrazine-2-carboxylate hydrochloride |
| TDH-070 | (R)-2-((4-fluorophenyl)amino)-2-oxo-1-phenylethyl 6-amino-1′,2′,3′,6′-tetrahydro-[3,4′- |
| | bipyridine]-5-carboxylate hydrochloride |
| TDH-071 | (S)-3-amino-N-(2-((4-fluorophenyl)amino)-2-oxo-1-phenylethyl)pyrazine-2-carboxamide |
| TDH-097 | (R)-2-((4-fluorophenyl)amino)-2-oxo-1-phenylethyl 6-(1-(1-acetylpiperidin-4-yl)-1H-pyrazol- |
| | 4-yl)-3-aminopyrazine-2-carboxylate |
| TDH-098 | tert-butyl (R)-4-(4-(5-amino-6-((2-((4-fluorophenyl)amino)-2-oxo-1- |
| | phenylethyl)carbamoyl)pyrazin-2-yl)-1H-pyrazol-1-yl)piperidine-1-carboxylate |
| TDH-099 | (R)-3-amino-N-(2-((4-fluorophenyl)amino)-2-oxo-1-phenylethyl)-6-(1-(piperidin-4-yl)-1H- |
| | pyrazol-4-yl)pyrazine-2-carboxamide hydrochloride |
| TDH-100 | (S)-2-((4-fluorophenyl)amino)-2-oxo-1-phenylethyl 3-amino-6-(1-(1-(2,2-dimethyl-4-oxo- |
| | 3,8,11-trioxa-5-azatridecan-13-yl)piperidin-4-yl)-1H-pyrazol-4-yl)pyrazine-2-carboxylate |
| TDH-106 | (R)-3-amino-N-(2-((4-fluorophenyl)amino)-2-oxo-1-phenylethyl)pyrazine-2-carboxamide |
| TDH-112 | (R)-2-((4-fluorophenyl)amino)-2-oxo-1-phenylethyl 2-amino-5-(1-(piperidin-4-yl)-1H- |
| | pyrazol-4-yl)nicotinate hydrochloride |
| TDH-180 | (R)-2-((4-fluorophenyl)amino)-2-oxo-1-phenylethyl 3-amino-6-(1-(1-(tert- |
| | butoxycarbonyl)piperidin-4-yl)-1H-1,2,3-triazol-4-yl)pyrazine-2-carboxylate |
| TDH-181 | (R)-2-((4-fluorophenyl)amino)-2-oxo-1-phenylethyl 3-amino-6-(1-(piperidin-4-yl)-1H-1,2,3- |
| | triazol-4-yl)pyrazine-2-carboxylate |
| TDH-182 | (R)-2-((4-fluorophenyl)amino)-2-oxo-1-phenylethyl 3-amino-6-(1-(1-methylpiperidin-4-yl)- |
| | 1H-1,2,3-triazol-4-yl)pyrazine-2-carboxylate |
| TDH-183 | (R)-2-((4-fluorophenyl)amino)-2-oxo-1-phenylethyl 3-amino-6-(6-(4-ethylpiperazin-1- |
| | yl)pyridin-3-yl)pyrazine-2-carboxylate |
| TDH-184 | (R)-2-((4-fluorophenyl)amino)-2-oxo-1-phenylethyl 6-(6-(4-acetylpiperazin-1-yl)pyridin-3- |
| | yl)-3-aminopyrazine-2-carboxylate |
| TDH-185 | (R)-2-((4-fluorophenyl)amino)-2-oxo-1-phenylethyl 6-(1-(1-acetylazetidin-3-yl)-1H-pyrazol- |
| | 4-yl)-3-aminopyrazine-2-carboxylate |
| TDH-227 | 2-((4-fluorophenyl)amino)-2-oxo-1-phenylethyl 3-amino-6-(1-((S)-1-(tert- |
| | butoxycarbonyl)pyrrolidin-3-yl)-1H-pyrazol-4-yl)pyrazine-2-carboxylate |
| TDH-228 | 2-((4-fluorophenyl)amino)-2-oxo-1-phenylethyl 6-(1-((S)-1-acetylpyrrolidin-3-yl)-1H- |
| | pyrazol-4-yl)-3-aminopyrazine-2-carboxylate |
| TDH-229 | 2-((4-fluorophenyl)amino)-2-oxo-1-phenylethyl 6-(1-((R)-1-acetylpyrrolidin-3-yl)-1H- |
| | pyrazol-4-yl)-3-aminopyrazine-2-carboxylate |
| TDH-313 | (R)-2-((4-fluorophenyl)amino)-2-oxo-1-phenylethyl 3-amino-6-bromopyrazine-2- |
| | carboxylate |
| TDH-314 | (R)-2-((3-chlorophenyl)amino)-2-oxo-1-phenylethyl 3-amino-6-(1-(piperidin-4-yl)-1H- |
| | pyrazol-4-yl)pyrazine-2-carboxylate hydrochloride |
| TDH-315 | (R)-2-((4-chlorophenyl)amino)-2-oxo-1-phenylethyl 3-amino-6-(1-(piperidin-4-yl)-1H- |
| | pyrazol-4-yl)pyrazine-2-carboxylate hydrochloride |
| TDH-316 | (R)-2-((3-methoxyphenyl)amino)-2-oxo-1-phenylethyl 3-amino-6-(1-(piperidin-4-yl)-1H- |
| | pyrazol-4-yl)pyrazine-2-carboxylate hydrochloride |
| TDH-317 | (R)-2-((4-methoxyphenyl)amino)-2-oxo-1-phenylethyl 3-amino-6-(1-(piperidin-4-yl)-1H- |
| | pyrazol-4-yl)pyrazine-2-carboxylate hydrochloride |
| TDH-318 | (R)-2-oxo-1-phenyl-2-(m-tolylamino)ethyl 3-amino-6-(1-(piperidin-4-yl)-1H-pyrazol-4- |
| | yl)pyrazine-2-carboxylate hydrochloride |
| TDH-319 | (R)-2-oxo-1-phenyl-2-(p-tolylamino)ethyl 3-amino-6-(1-(piperidin-4-yl)-1H-pyrazol-4- |
| | yl)pyrazine-2-carboxylate hydrochloride |
| TDH-320 | (R)-2-((4-fluorophenyl)amino)-2-oxo-1-(p-tolyl)ethyl 3-amino-6-(1-(piperidin-4-yl)-1H- |
| | pyrazol-4-yl)pyrazine-2-carboxylate hydrochloride |
| TDH-321 | (R)-2-((4-fluorophenyl)amino)-2-oxo-1-(p-tolyl)ethyl 3-amino-6-(1-(piperidin-4-yl)-1H- |
| | pyrazol-4-yl)pyrazine-2-carboxylate hydrochloride |
| TDH-322 | (R)-1-(4-chlorophenyl)-2-((4-fluorophenyl)amino)-2-oxoethyl 3-amino-6-(1-(piperidin-4-yl)- |
| | 1H-pyrazol-4-yl)pyrazine-2-carboxylate hydrochloride |
| TDH-323 | (R)-2-((4-fluorophenyl)amino)-2-oxo-1-(p-tolyl)ethyl 6-(1-(1-acetylpiperidin-4-yl)-1H- |
| | pyrazol-4-yl)-3-aminopyrazine-2-carboxylate |
| TDH-324 | (R)-1-(3-chlorophenyl)-2-((4-fluorophenyl)amino)-2-oxoethyl 6-(1-(1-acetylpiperidin-4-yl)- |
| | 1H-pyrazol-4-yl)-3-aminopyrazine-2-carboxylate |
| TDH-325 | (R)-1-(4-chlorophenyl)-2-((4-fluorophenyl)amino)-2-oxoethyl 6-(1-(1-acetylpiperidin-4-yl)- |
| | 1H-pyrazol-4-yl)-3-aminopyrazine-2-carboxylate |
| TDH-326 | (R)-2-((4-fluorophenyl)amino)-1-(4-methoxyphenyl)-2-oxoethyl 3-amino-6-(1-(piperidin-4- |
| | yl)-1H-pyrazol-4-yl)pyrazine-2-carboxylate hydrochloride |
| TDH-327 | (R)-2-((4-fluorophenyl)amino)-2-oxo-1-phenylethyl 3-amino-6-(4-chlorophenyl)pyrazine-2- |
| | carboxylate |
| TDH-329 | (R)-2-((4-fluorophenyl)amino)-2-oxo-1-phenylethyl 3-amino-6-(4- |
| | (methylsulfonyl)phenyl)pyrazine-2-carboxylate |
| TDH-335 | (R)-2-((4-fluorophenyl)amino)-2-oxo-1-phenylethyl 3-amino-6-(pyridin-4-yl)pyrazine-2- |
| | carboxylate |
| TDH-424 | (R)-2-((4-methylcyclohexyl)amino)-2-oxo-1-phenylethyl 3-amino-6-(1-(piperidin-4-yl)-1H- |
| | pyrazol-4-yl)pyrazine-2-carboxylate hydrochloride |
| TDH-444 | (R)-2-((1-methylpiperidin-4-yl)amino)-2-oxo-1-phenylethyl 3-amino-6-(1-(piperidin-4-yl)- |
| | 1H-pyrazol-4-yl)pyrazine-2-carboxylate hydrochloride |
| TDH-455 | (R)-2-(4-methylpiperazin-1-yl)-2-oxo-1-phenylethyl 3-amino-6-(1-(piperidin-4-yl)-1H- |
| | pyrazol-4-yl)pyrazine-2-carboxylate hydrochloride |
| TDH-456 | (R)-2-((3-fluorophenyl)amino)-2-oxo-1-phenylethyl 3-amino-6-(1-(piperidin-4-yl)-1H- |
| | pyrazol-4-yl)pyrazine-2-carboxylate hydrochloride |
| TDH-457 | (R)-2-((4-fluorobenzyl)amino)-2-oxo-1-phenylethyl 3-amino-6-(1-(piperidin-4-yl)-1H- |
| | pyrazol-4-yl)pyrazine-2-carboxylate hydrochloride |
| TDH-458 | (R)-2-((2-fluorophenyl)amino)-2-oxo-1-phenylethyl 3-amino-6-(1-(piperidin-4-yl)-1H- |
| | pyrazol-4-yl)pyrazine-2-carboxylate hydrochloride |
| TDH-459 | (R)-2-oxo-1-phenyl-2-((4-(trifluoromethyl)phenyl)amino)ethyl 3-amino-6-(1-(piperidin-4-yl)- |
| | 1H-pyrazol-4-yl)pyrazine-2-carboxylate hydrochloride |
| TDH-460 | (R)-2-oxo-1-phenyl-2-((3-(trifluoromethyl)phenyl)amino)ethyl 3-amino-6-(1-(piperidin-4-yl)- |
| | 1H-pyrazol-4-yl)pyrazine-2-carboxylate hydrochloride |
| TDH-461 | (R)-2-((4-(4-methylpiperazin-1-yl)phenyl)amino)-2-oxo-1-phenylethyl 3-amino-6-(1- |
| | (piperidin-4-yl)-1H-pyrazol-4-yl)pyrazine-2-carboxylate hydrochloride |
| TDH-462 | (R)-2-((3-(4-methylpiperazin-1-yl)phenyl)amino)-2-oxo-1-phenylethyl 3-amino-6-(1- |
| | (piperidin-4-yl)-1H-pyrazol-4-yl)pyrazine-2-carboxylate hydrochloride |
| TDH-463 | (R)-2-oxo-1-phenyl-2-(phenylamino)ethyl 3-amino-6-(1-(piperidin-4-yl)-1H-pyrazol-4- |
| | yl)pyrazine-2-carboxylate hydrochloride |
| TDH-464 | (R)-2-((4-fluoro-3-methylphenyl)amino)-2-oxo-1-phenylethyl 3-amino-6-(1-(piperidin-4-yl)- |
| | 1H-pyrazol-4-yl)pyrazine-2-carboxylate hydrochloride |
| TDH-465 | (R)-1-(2-chlorophenyl)-2-((4-fluorophenyl)amino)-2-oxoethyl 3-amino-6-(1-(piperidin-4-yl)- |
| | 1H-pyrazol-4-yl)pyrazine-2-carboxylate hydrochloride |
| TDH-468 | (R)-2-oxo-1-phenyl-2-(pyridin-3-ylamino)ethyl 3-amino-6-(1-(piperidin-4-yl)-1H-pyrazol-4- |
| | yl)pyrazine-2-carboxylate hydrochloride |
| TDH-469 | (R)-2-((3-(dimethylamino)phenyl)amino)-2-oxo-1-phenylethyl 3-amino-6-(1-(piperidin-4-yl)- |
| | 1H-pyrazol-4-yl)pyrazine-2-carboxylate hydrochloride |
| TDH-470 | (R)-2-((4-(dimethylamino)phenyl)amino)-2-oxo-1-phenylethyl 3-amino-6-(1-(piperidin-4-yl)- |
| | 1H-pyrazol-4-yl)pyrazine-2-carboxylate hydrochloride |
| TDH-471 | (R)-1-((4-fluorophenyl)amino)-3-methyl-1-oxobutan-2-yl 3-amino-6-(1-(piperidin-4-yl)-1H- |
| | pyrazol-4-yl)pyrazine-2-carboxylate hydrochloride |
| TDH-479 | (R)-2-oxo-1-phenyl-2-(pyridin-4-ylamino)ethyl 3-amino-6-(1-(piperidin-4-yl)-1H-pyrazol-4- |
| | yl)pyrazine-2-carboxylate--2,2,2-trifluoroacetaldehyde |
| TDH-488 | (R)-2-((3,4-difluorophenyl)amino)-2-oxo-1-phenylethyl 3-amino-6-(1-(piperidin-4-yl)-1H- |
| | pyrazol-4-yl)pyrazine-2-carboxylate hydrochloride |
| TDH-489 | 1-(4-fluorophenyl)-2-((4-fluorophenyl)amino)-2-oxoethyl 3-amino-6-(1-(piperidin-4-yl)-1H- |
| | pyrazol-4-yl)pyrazine-2-carboxylate hydrochloride |
| TDH-490 | (R)-3-amino-N-(2-((4-fluorophenyl)amino)-2-oxo-1-phenylethyl)-N-methyl-6-(1-(piperidin-4- |
| | yl)-1H-pyrazol-4-yl)pyrazine-2-carboxamide hydrochloride |

Non-limiting examples of compounds of Chemical Formula 2 according to the present invention include the compounds in Table 2 below and pharmaceutically acceptable salts thereof.

TABLE 2

| Compound | IUPAC Name |
|---|---|
| TDH-072 | (R)-2-((4-fluorophenyl)amino)-2-oxo-1-phenylethyl 6-amino-1′-(6-(2-((2-(2,6- |
| | dioxopiperidin-3-yl)-1,3-dioxoisoindolin-5-yl)amino)acetamido)hexanoyl)-1′,2′,3′,6′- |
| | tetrahydro-[3,4′-bipyridine]-5-carboxylate |
| TDH-073 | (R)-2-((4-fluorophenyl)amino)-2-oxo-1-phenylethyl 6-amino-1′-(6-((2-(2,6-dioxopiperidin-3- |
| | yl)-1,3-dioxoisoindolin-4-yl)amino)hexanoyl)-1′,2′,3′,6′-tetrahydro-[3,4′-bipyridine]-5- |
| | carboxylate |
| TDH-074 | (R)-2-((4-fluorophenyl)amino)-2-oxo-1-phenylethyl 6-amino-1′-(6-(1-(2-(2,6-dioxopiperidin- |
| | 3-yl)-1,3-dioxoisoindolin-5-yl)piperidine-4-carboxamido)hexanoyl)-1′,2′,3′,6′-tetrahydro- |
| | [3,4′-bipyridine]-5-carboxylate |
| TDH-075 | (R)-2-((4-fluorophenyl)amino)-2-oxo-1-phenylethyl 6-amino-1′-(6-((2-(2,6-dioxopiperidin-3- |
| | yl)-1,3-dioxoisoindolin-5-yl)amino)hexanoyl)-1′,2′,3′,6′-tetrahydro-[3,4′-bipyridine]-5- |
| | carboxylate |
| TDH-078 | (R)-2-((4-fluorophenyl)amino)-2-oxo-1-phenylethyl 6-amino-1′-(6-(1-(2-(2,6-dioxopiperidin- |
| | 3-yl)-1,3-dioxoisoindolin-4-yl)azetidine-3-carboxamido)hexanoyl)-1′,2′,3′,6′-tetrahydro- |
| | [3,4′-bipyridine]-5-carboxylate |
| TDH-079 | (R)-2-((4-fluorophenyl)amino)-2-oxo-1-phenylethyl 6-amino-1′-(6-(2-((2-(2,6- |
| | dioxopiperidin-3-yl)-1,3-dioxoisoindolin-4-yl)amino)acetamido)hexanoyl)-1′,2′,3′,6′- |
| | tetrahydro-[3,4′-bipyridine]-5-carboxylate |
| TDH-080 | (R)-2-((4-fluorophenyl)amino)-2-oxo-1-phenylethyl 6-amino-1′-(6-(1-(2-(2,6-dioxopiperidin- |
| | 3-yl)-1,3-dioxoisoindolin-4-yl)piperidine-4-carboxamido)hexanoyl)-1′,2′,3′,6′-tetrahydro- |
| | [3,4′-bipyridine]-5-carboxylate |
| TDH-081 | (R)-2-((4-fluorophenyl)amino)-2-oxo-1-phenylethyl 6-amino-1′-(6-(2-((2-(2,6- |
| | dioxopiperidin-3-yl)-1,3-dioxoisoindolin-4-yl)oxy)acetamido)hexanoyl)-1′,2′,3′,6′-tetrahydro- |
| | [3,4′-bipyridine]-5-carboxylate |
| TDH-082 | (R)-2-((4-fluorophenyl)amino)-2-oxo-1-phenylethyl 6-amino-1′-(2-(2-(2-((2-(2,6- |
| | dioxopiperidin-3-yl)-1,3-dioxoisoindolin-4-yl)amino)ethoxy)ethoxy)acetyl)-1′,2′,3′,6′- |
| | tetrahydro-[3,4′-bipyridine]-5-carboxylate |
| TDH-083 | (R)-2-((4-fluorophenyl)amino)-2-oxo-1-phenylethyl 6-amino-1′-(2-(2-(2-((2-(2,6- |
| | dioxopiperidin-3-yl)-1,3-dioxoisoindolin-5-yl)amino)ethoxy)ethoxy)acetyl)-1′,2′,3′,6′- |
| | tetrahydro-[3,4′-bipyridine]-5-carboxylate |
| TDH-084 | (R)-2-((4-fluorophenyl)amino)-2-oxo-1-phenylethyl 6-amino-1′-(2-(2-(2-(2-((2-(2,6- |
| | dioxopiperidin-3-yl)-1,3-dioxoisoindolin-4-yl)amino)acetamido)ethoxy)ethoxy)acetyl)- |
| | 1′,2′,3′,6′-tetrahydro-[3,4′-bipyridine]-5-carboxylate |
| TDH-085 | (R)-2-((4-fluorophenyl)amino)-2-oxo-1-phenylethyl 6-amino-1′-(2-(2-(2-(2-((2-(2,6- |
| | dioxopiperidin-3-yl)-1,3-dioxoisoindolin-5-yl)amino)acetamido)ethoxy)ethoxy)acetyl)- |
| | 1′,2′,3′,6′-tetrahydro-[3,4′-bipyridine]-5-carboxylate |
| TDH-086 | (R)-2-((4-fluorophenyl)amino)-2-oxo-1-phenylethyl 6-amino-1′-(2-(2-(2-(1-(2-(2,6- |
| | dioxopiperidin-3-yl)-1,3-dioxoisoindolin-4-yl)azetidine-3- |
| | carboxamido)ethoxy)ethoxy)acetyl)-1′,2′, 3′,6′-tetrahydro-[3,4′-bipyridine]-5-carboxylate |
| TDH-087 | (R)-2-((4-fluorophenyl)amino)-2-oxo-1-phenylethyl 6-amino-1′-(2-(2-(2-(1-(2-(2,6- |
| | dioxopiperidin-3-yl)-1,3-dioxoisoindolin-5-yl)azetidine-3- |
| | carboxamido)ethoxy)ethoxy)acetyl)-1′,2′,3′,6′-tetrahydro-[3,4′-bipyridine]-5-carboxylate |
| TDH-088 | (R)-2-((4-fluorophenyl)amino)-2-oxo-1-phenylethyl 6-amino-1′-(2-(2-(2-(1-(2-(2,6- |
| | dioxopiperidin-3-yl)-1,3-dioxoisoindolin-4-yl)piperidine-4- |
| | carboxamido)ethoxy)ethoxy)acetyl)-1′,2′,3′,6′-tetrahydro-[3,4′-bipyridine]-5-carboxylate |
| TDH-089 | (R)-2-((4-fluorophenyl)amino)-2-oxo-1-phenylethyl 6-amino-1′-(2-(2-(2-(1-(2-(2,6- |
| | dioxopiperidin-3-yl)-1,3-dioxoisoindolin-5-yl)piperidine-4- |
| | carboxamido)ethoxy)ethoxy)acetyl)-1′,2′,3′,6′-tetrahydro-[3,4′-bipyridine]-5-carboxylate |
| TDH-092 | (R)-2-((4-fluorophenyl)amino)-2-oxo-1-phenylethyl 6-amino-1′-(6-(1-(2-(2,6-dioxopiperidin- |
| | 3-yl)-1,3-dioxoisoindolin-5-yl)azetidine-3-carboxamido)hexanoyl)-1′,2′,3′,6′-tetrahydro- |
| | [3,4′-bipyridine]-5-carboxylate |
| TDH-095 | (R)-2-((4-fluorophenyl)amino)-2-oxo-1-phenylethyl 3-amino-6-(1-(1-(10-((2-(2,6- |
| | dioxopiperidin-3-yl)-1,3-dioxoisoindolin-4-yl)amino)decyl)piperidin-4-yl)-1H-pyrazol-4- |
| | yl)pyrazine-2-carboxylate |
| TDH-096 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-4-yl)amino)acetamido)ethoxy)ethyl)piperidin-4-yl)- |
| | 1H-pyrazol-4-yl)pyrazine-2-carboxylate |
| TDH-101 | (R)-2-((4-fluorophenyl)amino)-2-oxo-1-phenylethyl 3-amino-6-(1-(1-(6-(2-((2-(2,6- |
| | dioxopiperidin-3-yl)-1,3-dioxoisoindolin-4-yl)amino)acetamido)hexyl)piperidin-4-yl)-1H- |
| | pyrazol-4-yl)pyrazine-2-carboxylate |
| TDH-102 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-4- |
| | yl)amino)acetamido)ethoxy)ethoxy)ethyl)piperidin-4-yl)-1H-pyrazol-4-yl)pyrazine-2- |
| | carboxylate |
| TDH-103 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-5- |
| | yl)amino)acetamido)ethoxy)ethoxy)ethyl)piperidin-4-yl)-1H-pyrazol-4-yl)pyrazine-2- |
| | carboxylate |
| TDH-104 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-4-yl)amino)ethoxy)ethoxy)acetyl)piperidin-4-yl)-1H- |
| | pyrazol-4-yl)pyrazine-2-carboxylate |
| TDH-105 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-5-yl)amino)ethoxy)ethoxy)acetyl)piperidin-4-yl)-1H- |
| | pyrazol-4-yl)pyrazine-2-carboxylate |
| TDH-107 | (R)-2-((4-fluorophenyl)amino)-2-oxo-1-phenylethyl 3-amino-6-(1-(2-(2-(2-(2-((2-(2,6- |
| | dioxopiperidin-3-yl)-1,3-dioxoisoindolin-5-yl)amino)acetamido)ethoxy)ethoxy)ethyl)- |
| | 1,2,3,6-tetrahydropyridin-4-yl)pyrazine-2-carboxylate |
| TDH-108 | (R)-2-((4-fluorophenyl)amino)-2-oxo-1-phenylethyl 3-amino-6-(1-(1-(6-(1-(2-(2,6- |
| | dioxopiperidin-3-yl)-1,3-dioxoisoindolin-4-yl)piperidine-4-carboxamido)hexyl)piperidin-4- |
| | yl)-1H-pyrazol-4-yl)pyrazine-2-carboxylate |
| TDH-109 | (R)-2-((4-fluorophenyl)amino)-2-oxo-1-phenylethyl 3-amino-6-(1-(1-(6-(1-(2-(2,6- |
| | dioxopiperidin-3-yl)-1,3-dioxoisoindolin-5-yl)piperidine-4-carboxamido)hexyl)piperidin-4- |
| | yl)-1H-pyrazol-4-yl)pyrazine-2-carboxylate |
| TDH-110 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-4-yl)piperidine-4- |
| | carboxamido)ethoxy)ethoxy)ethyl)piperidin-4-yl)-1H-pyrazol-4-yl)pyrazine-2-carboxylate |
| TDH-111 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-5-yl)piperidine-4- |
| | carboxamido)ethoxy)ethoxy)ethyl)piperidin-4-yl)-1H-pyrazol-4-yl)pyrazine-2-carboxylate |
| TDH-113 | (R)-2-((4-fluorophenyl)amino)-2-oxo-1-phenylethyl 3-amino-6-(1-(1-(6-(1-(2-(2,6- |
| | dioxopiperidin-3-yl)-1,3-dioxoisoindolin-4-yl)azetidine-3-carboxamido)hexyl)piperidin-4-yl)- |
| | 1H-pyrazol-4-yl)pyrazine-2-carboxylate |
| TDH-114 | (R)-2-((4-fluorophenyl)amino)-2-oxo-1-phenylethyl 3-amino-6-(1-(1-(6-(1-(2-(2,6- |
| | dioxopiperidin-3-yl)-1,3-dioxoisoindolin-5-yl)azetidine-3-carboxamido)hexyl)piperidin-4-yl)- |
| | 1H-pyrazol-4-yl)pyrazine-2-carboxylate |
| TDH-115 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-4-yl)azetidine-3- |
| | carboxamido)ethoxy)ethoxy)ethyl)piperidin-4-yl)-1H-pyrazol-4-yl)pyrazine-2-carboxylate |
| TDH-116 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-5-yl)azetidine-3- |
| | carboxamido)ethoxy)ethoxy)ethyl)piperidin-4-yl)-1H-pyrazol-4-yl)pyrazine-2-carboxylate |
| TDH-119 | (R)-2-((4-fluorophenyl)amino)-2-oxo-1-phenylethyl 3-amino-6-(1-(1-(6-((2-(2,6- |
| | dioxopiperidin-3-yl)-1,3-dioxoisoindolin-4-yl)amino)hexyl)piperidin-4-yl)-1H-pyrazol-4- |
| | yl)pyrazine-2-carboxylate |
| TDH-120 | (R)-2-((4-fluorophenyl)amino)-2-oxo-1-phenylethyl 3-amino-6-(1-(1-(6-((2-(2,6- |
| | dioxopiperidin-3-yl)-1,3-dioxoisoindolin-5-yl)amino)hexyl)piperidin-4-yl)-1H-pyrazol-4- |
| | yl)pyrazine-2-carboxylate |
| TDH-121 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-4-yl)amino)ethoxy)ethoxy)ethyl)piperidin-4-yl)-1H- |
| | pyrazol-4-yl)pyrazine-2-carboxylate |
| TDH-122 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-5-yl)amino)ethoxy)ethoxy)ethyl)piperidin-4-yl)-1H- |
| | pyrazol-4-yl)pyrazine-2-carboxylate |
| TDH-123 | (R)-2-((4-fluorophenyl)amino)-2-oxo-1-phenylethyl 3-amino-6-(1-(1-(6-(2-((2-(2,6- |
| | dioxopiperidin-3-yl)-1,3-dioxoisoindolin-5-yl)amino)acetamido)hexanoyl)piperidin-4-yl)-1H- |
| | pyrazol-4-yl)pyrazine-2-carboxylate |
| TDH-124 | (R)-2-((4-fluorophenyl)amino)-2-oxo-1-phenylethyl 3-amino-6-(1-(1-(6-(2-((2-(2,6- |
| | dioxopiperidin-3-yl)-1,3-dioxoisoindolin-4-yl)amino)acetamido)hexanoyl)piperidin-4-yl)-1H- |
| | pyrazol-4-yl)pyrazine-2-carboxylate |
| TDH-125 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-5-yl)amino)acetamido)ethoxy)ethyl)piperidin-4-yl)- |
| | 1H-pyrazol-4-yl)pyrazine-2-carboxylate |
| TDH-126 | (R)-2-((4-fluorophenyl)amino)-2-oxo-1-phenylethyl 3-amino-6-(1-(1-(2-(2-(1-(2-(2,6- |
| | dioxopiperidin-3-yl)-1,3-dioxoisoindolin-4-yl)azetidine-3- |
| | carboxamido)ethoxy)ethyl)piperidin-4-yl)-1H-pyrazol-4-yl)pyrazine-2-carboxylate |
| TDH-127 | (R)-2-((4-fluorophenyl)amino)-2-oxo-1-phenylethyl 3-amino-6-(1-(1-(2-(2-(1-(2-(2,6- |
| | dioxopiperidin-3-yl)-1,3-dioxoisoindolin-5-yl)azetidine-3- |
| | carboxamido)ethoxy)ethyl)piperidin-4-yl)-1H-pyrazol-4-yl)pyrazine-2-carboxylate |
| TDH-128 | (R)-2-((4-fluorophenyl)amino)-2-oxo-1-phenylethyl 3-amino-6-(1-(1-(2-(2-(1-(2-(2,6- |
| | dioxopiperidin-3-yl)-1,3-dioxoisoindolin-4-yl)piperidine-4- |
| | carboxamido)ethoxy)ethyl)piperidin-4-yl)-1H-pyrazol-4-yl)pyrazine-2-carboxylate |
| TDH-129 | (R)-2-((4-fluorophenyl)amino)-2-oxo-1-phenylethyl 3-amino-6-(1-(1-(2-(2-(1-(2-(2,6- |
| | dioxopiperidin-3-yl)-1,3-dioxoisoindolin-5-yl)piperidine-4- |
| | carboxamido)ethoxy)ethyl)piperidin-4-yl)-1H-pyrazol-4-yl)pyrazine-2-carboxylate |
| TDH-130 | (R)-2-((4-fluorophenyl)amino)-2-oxo-1-phenylethyl 3-amino-6-(1-(1-(2-(2-((2-(2,6- |
| | dioxopiperidin-3-yl)-1,3-dioxoisoindolin-4-yl)amino)ethoxy)ethyl)piperidin-4-yl)-1H-pyrazol- |
| | 4-yl)pyrazine-2-carboxylate |
| TDH-131 | (R)-2-((4-fluorophenyl)amino)-2-oxo-1-phenylethyl 3-amino-6-(1-(1-(2-(2-((2-(2,6- |
| | dioxopiperidin-3-yl)-1,3-dioxoisoindolin-5-yl)amino)ethoxy)ethyl)piperidin-4-yl)-1H-pyrazol- |
| | 4-yl)pyrazine-2-carboxylate |
| TDH-132 | (R)-2-((4-fluorophenyl)amino)-2-oxo-1-phenylethyl 3-amino-6-(1-(1-(6-(2-((2-(2,6- |
| | dioxopiperidin-3-yl)-1,3-dioxoisoindolin-5-yl)amino)acetamido)hexyl)piperidin-4-yl)-1H- |
| | pyrazol-4-yl)pyrazine-2-carboxylate |
| TDH-133 | (R)-2-((4-fluorophenyl)amino)-2-oxo-1-phenylethyl 3-amino-6-(1-(1-(6-(2-((2-(2,6- |
| | dioxopiperidin-3-yl)-1,3-dioxoisoindolin-4-yl)oxy)acetamido)hexyl)piperidin-4-yl)-1H- |
| | pyrazol-4-yl)pyrazine-2-carboxylate |
| TDH-134 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-4-yl)oxy)acetamido)ethoxy)ethyl)piperidin-4-yl)-1H- |
| | pyrazol-4-yl)pyrazine-2-carboxylate |
| TDH-135 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-4-yl)oxy)acetamido)ethoxy)ethoxy)ethyl)piperidin- |
| | 4-yl)-1H-pyrazol-4-yl)pyrazine-2-carboxylate |
| TDH-136 | (R)-2-((4-fluorophenyl)amino)-2-oxo-1-phenylethyl 3-amino-6-(1-(1-((1-((2-(2,6- |
| | dioxopiperidin-3-yl)-1,3-dioxoisoindolin-4-yl)glycyl)piperidin-4-yl)methyl)piperidin-4-yl)-1H- |
| | pyrazol-4-yl)pyrazine-2-carboxylate |
| TDH-137 | (R)-2-((4-fluorophenyl)amino)-2-oxo-1-phenylethyl 3-amino-6-(1-(1-((1-((2-(2,6- |
| | dioxopiperidin-3-yl)-1,3-dioxoisoindolin-5-yl)glycyl)piperidin-4-yl)methyl)piperidin-4-yl)-1H- |
| | pyrazol-4-yl)pyrazine-2-carboxylate |
| TDH-138 | (R)-2-((4-fluorophenyl)amino)-2-oxo-1-phenylethyl 3-amino-6-(1-(1-((1-(1-(2-(2,6- |
| | dioxopiperidin-3-yl)-1,3-dioxoisoindolin-4-yl)azetidine-3-carbonyl)piperidin-4- |
| | yl)methyl)piperidin-4-yl)-1H-pyrazol-4-yl)pyrazine-2-carboxylate |
| TDH-139 | (R)-2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-4-yl)piperidin-4-yl)-1-oxo-5,8,11-trioxa-2- |
| | azatridecan-13-yl)piperidin-4-yl)-1H-pyrazol-4-yl)pyrazine-2-carboxylate |
| TDH-140 | (R)-2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-5-yl)piperidin-4-yl)-1-oxo-5,8,11-trioxa-2- |
| | azatridecan-13-yl)piperidin-4-yl)-1H-pyrazol-4-yl)pyrazine-2-carboxylate |
| TDH-141 | (R)-2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-4-yl)azetidin-3-yl)-1-oxo-5,8,11-trioxa-2- |
| | azatridecan-13-yl)piperidin-4-yl)-1H-pyrazol-4-yl)pyrazine-2-carboxylate |
| TDH-142 | (R)-2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-5-yl)azetidin-3-yl)-1-oxo-5,8,11-trioxa-2- |
| | azatridecan-13-yl)piperidin-4-yl)-1H-pyrazol-4-yl)pyrazine-2-carboxylate |
| TDH-143 | (R)-2-((4-fluorophenyl)amino)-2-oxo-1-phenylethyl 3-amino-6-(1-(1-(1-((2-(2,6- |
| | dioxopiperidin-3-yl)-1,3-dioxoisoindolin-4-yl)amino)-2-oxo-6,9,12-trioxa-3-azatetradecan- |
| | 14-yl)piperidin-4-yl)-1H-pyrazol-4-yl)pyrazine-2-carboxylate |
| TDH-144 | (R)-2-((4-fluorophenyl)amino)-2-oxo-1-phenylethyl 3-amino-6-(1-(1-(1-((2-(2,6- |
| | dioxopiperidin-3-yl)-1,3-dioxoisoindolin-5-yl)amino)-2-oxo-6,9,12-trioxa-3-azatetradecan- |
| | 14-yl)piperidin-4-yl)-1H-pyrazol-4-yl)pyrazine-2-carboxylate |
| TDH-145 | (R)-2-((4-fluorophenyl)amino)-2-oxo-1-phenylethyl 3-amino-6-(1-(1-((1-(1-(2-(2,6- |
| | dioxopiperidin-3-yl)-1,3-dioxoisoindolin-5-yl)azetidine-3-carbonyl)piperidin-4- |
| | yl)methyl)piperidin-4-yl)-1H-pyrazol-4-yl)pyrazine-2-carboxylate |
| TDH-146 | (R)-2-((4-fluorophenyl)amino)-2-oxo-1-phenylethyl 3-amino-6-(1-(1-((1-(1-(2-(2,6- |
| | dioxopiperidin-3-yl)-1,3-dioxoisoindolin-4-yl)piperidine-4-carbonyl)piperidin-4- |
| | yl)methyl)piperidin-4-yl)-1H-pyrazol-4-yl)pyrazine-2-carboxylate |
| TDH-147 | (R)-2-((4-fluorophenyl)amino)-2-oxo-1-phenylethyl 3-amino-6-(1-(1-((1-(1-(2-(2,6- |
| | dioxopiperidin-3-yl)-1,3-dioxoisoindolin-5-yl)piperidine-4-carbonyl)piperidin-4- |
| | yl)methyl)piperidin-4-yl)-1H-pyrazol-4-yl)pyrazine-2-carboxylate |
| TDH-148 | (R)-2-((4-fluorophenyl)amino)-2-oxo-1-phenylethyl 3-amino-6-(1-(1-((1-(2-((2-(2,6- |
| | dioxopiperidin-3-yl)-1,3-dioxoisoindolin-4-yl)oxy)acetyl)piperidin-4-yl)methyl)piperidin-4-yl)- |
| | 1H-pyrazol-4-yl)pyrazine-2-carboxylate |
| TDH-149 | (R)-2-((4-fluorophenyl)amino)-2-oxo-1-phenylethyl 3-amino-6-(1-(1-((1-(2-(2,6- |
| | dioxopiperidin-3-yl)-1,3-dioxoisoindolin-4-yl)piperidin-4-yl)methyl)piperidin-4-yl)-1H- |
| | pyrazol-4-yl)pyrazine-2-carboxylate |
| TDH-150 | (R)-2-((4-fluorophenyl)amino)-2-oxo-1-phenylethyl 3-amino-6-(1-(1-((1-(2-(2,6- |
| | dioxopiperidin-3-yl)-1,3-dioxoisoindolin-5-yl)piperidin-4-yl)methyl)piperidin-4-yl)-1H- |
| | pyrazol-4-yl)pyrazine-2-carboxylate |
| TDH-151 | (R)-2-((4-fluorophenyl)amino)-2-oxo-1-phenylethyl 3-amino-6-(1-(1-((1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)-1H-pyrazol-4-yl)pyrazine-2-carboxylate |
| TDH-152 | (R)-2-((4-fluorophenyl)amino)-2-oxo-1-phenylethyl 3-amino-6-(1-(1-((1-((3-(2,6- |
| | dioxopiperidin-3-yl)-4-oxo-3,4-dihydrobenzo[d][1,2,3]triazin-6-yl)glycyl)piperidin-4- |
| | yl)methyl)piperidin-4-yl)-1H-pyrazol-4-yl)pyrazine-2-carboxylate |
| TDH-153 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-4-yl)amino)ethoxy)ethoxy)ethoxy)ethyl)piperidin-4- |
| | yl)-1H-pyrazol-4-yl)pyrazine-2-carboxylate |
| TDH-154 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-5-yl)amino)ethoxy)ethoxy)ethoxy)ethyl)piperidin-4- |
| | yl)-1H-pyrazol-4-yl)pyrazine-2-carboxylate |
| TDH-155 | (R)-2-((4-fluorophenyl)amino)-2-oxo-1-phenylethyl 3-amino-6-(1-(1-(1-((2-(2,6- |
| | dioxopiperidin-3-yl)-1,3-dioxoisoindolin-4-yl)oxy)-2-oxo-6,9,12-trioxa-3-azatetradecan-14- |
| | yl)piperidin-4-yl)-1H-pyrazol-4-yl)pyrazine-2-carboxylate |
| TDH-156 | (R)-2-((4-fluorophenyl)amino)-2-oxo-1-phenylethyl 3-amino-6-(1-(1-(10-(1-(2-(2,6- |
| | dioxopiperidin-3-yl)-1,3-dioxoisoindolin-4-yl)piperidine-4-carboxamido)decyl)piperidin-4- |
| | yl)-1H-pyrazol-4-yl)pyrazine-2-carboxylate |
| TDH-157 | (R)-2-((4-fluorophenyl)amino)-2-oxo-1-phenylethyl 3-amino-6-(1-(1-(10-(1-(2-(2,6- |
| | dioxopiperidin-3-yl)-1,3-dioxoisoindolin-5-yl)piperidine-4-carboxamido)decyl)piperidin-4- |
| | yl)-1H-pyrazol-4-yl)pyrazine-2-carboxylate |
| TDH-158 | (R)-2-((4-fluorophenyl)amino)-2-oxo-1-phenylethyl 3-amino-6-(1-(1-(10-(1-(2-(2,6- |
| | dioxopiperidin-3-yl)-1,3-dioxoisoindolin-4-yl)azetidine-3-carboxamido)decyl)piperidin-4-yl)- |
| | 1H-pyrazol-4-yl)pyrazine-2-carboxylate |
| TDH-159 | (R)-2-((4-fluorophenyl)amino)-2-oxo-1-phenylethyl 3-amino-6-(1-(1-(10-(1-(2-(2,6- |
| | dioxopiperidin-3-yl)-1,3-dioxoisoindolin-5-yl)azetidine-3-carboxamido)decyl)piperidin-4-yl)- |
| | 1H-pyrazol-4-yl)pyrazine-2-carboxylate |
| TDH-160 | (R)-2-((4-fluorophenyl)amino)-2-oxo-1-phenylethyl 3-amino-6-(1-(1-(10-((2-(2,6- |
| | dioxopiperidin-3-yl)-1,3-dioxoisoindolin-5-yl)amino)decyl)piperidin-4-yl)-1H-pyrazol-4- |
| | yl)pyrazine-2-carboxylate |
| TDH-161 | (R)-2-((4-fluorophenyl)amino)-2-oxo-1-phenylethyl 3-amino-6-(1-(1-(10-(2-((2-(2,6- |
| | dioxopiperidin-3-yl)-1,3-dioxoisoindolin-4-yl)amino)acetamido)decyl)piperidin-4-yl)-1H- |
| | pyrazol-4-yl)pyrazine-2-carboxylate |
| TDH-162 | (R)-2-((4-fluorophenyl)amino)-2-oxo-1-phenylethyl 3-amino-6-(1-(1-(10-(2-((2-(2,6- |
| | dioxopiperidin-3-yl)-1,3-dioxoisoindolin-5-yl)amino)acetamido)decyl)piperidin-4-yl)-1H- |
| | pyrazol-4-yl)pyrazine-2-carboxylate |
| TDH-163 | (R)-2-((4-fluorophenyl)amino)-2-oxo-1-phenylethyl 3-amino-6-(1-(1-(10-(2-((2-(2,6- |
| | dioxopiperidin-3-yl)-1,3-dioxoisoindolin-4-yl)oxy)acetamido)decyl)piperidin-4-yl)-1H- |
| | pyrazol-4-yl)pyrazine-2-carboxylate |
| TDH-164 | (R)-2-((4-fluorophenyl)amino)-2-oxo-1-phenylethyl 3-amino-6-(1-(1-(10-(2-((2-(2,6- |
| | dioxopiperidin-3-yl)-1,3-dioxoisoindolin-5-yl)oxy)acetamido)decyl)piperidin-4-yl)-1H- |
| | pyrazol-4-yl)pyrazine-2-carboxylate |
| TDH-165 | (R)-2-((4-fluorophenyl)amino)-2-oxo-1-phenylethyl 3-amino-6-(1-(1-(6-(2-((2-(2,6- |
| | dioxopiperidin-3-yl)-1,3-dioxoisoindolin-5-yl)oxy)acetamido)hexyl)piperidin-4-yl)-1H- |
| | pyrazol-4-yl)pyrazine-2-carboxylate |
| TDH-166 | (R)-2-((4-fluorophenyl)amino)-2-oxo-1-phenylethyl 3-amino-6-(1-(1-(1-((2-(2,6- |
| | dioxopiperidin-3-yl)-1,3-dioxoisoindolin-5-yl)oxy)-2-oxo-6,9,12-trioxa-3-azatetradecan-14- |
| | yl)piperidin-4-yl)-1H-pyrazol-4-yl)pyrazine-2-carboxylate |
| TDH-167 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-5-yl)oxy)acetamido)ethoxy)ethoxy)ethyl)piperidin- |
| | 4-yl)-1H-pyrazol-4-yl)pyrazine-2-carboxylate |
| TDH-168 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-5-yl)oxy)acetamido)ethoxy)ethyl)piperidin-4-yl)-1H- |
| | pyrazol-4-yl)pyrazine-2-carboxylate |
| TDH-169 | (R)-2-((4-fluorophenyl)amino)-2-oxo-1-phenylethyl 3-amino-6-(1-(1-((1-(2-((3-(2,6- |
| | dioxopiperidin-3-yl)-4-oxo-3,4-dihydrobenzo[d][1,2,3]triazin-6-yl)oxy)acetyl)piperidin-4- |
| | yl)methyl)piperidin-4-yl)-1H-pyrazol-4-yl)pyrazine-2-carboxylate |
| TDH-170 | (R)-2-((4-fluorophenyl)amino)-2-oxo-1-phenylethyl 3-amino-6-(1-(1-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)-1H-pyrazol-4-yl)pyrazine-2-carboxylate |
| TDH-171 | (R)-2-((4-fluorophenyl)amino)-2-oxo-1-phenylethyl 3-amino-6-(1-(1-((1-(2-((2-(2,6- |
| | dioxopiperidin-3-yl)-1,3-dioxoisoindolin-5-yl)oxy)acetyl)piperidin-4-yl)methyl)piperidin-4-yl)- |
| | 1H-pyrazol-4-yl)pyrazine-2-carboxylate |
| TDH-172 | (R)-2-((4-fluorophenyl)amino)-2-oxo-1-phenylethyl 3-amino-6-(1-(1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)-1H-pyrazol-4-yl)pyrazine-2-carboxylate |
| TDH-173 | (R)-2-((4-fluorophenyl)amino)-2-oxo-1-phenylethyl 3-amino-6-(1-(1-(10-(2-((3-(2,6- |
| | dioxopiperidin-3-yl)-4-oxo-3,4-dihydrobenzo[d][1,2,3]triazin-6- |
| | yl)amino)acetamido)decyl)piperidin-4-yl)-1H-pyrazol-4-yl)pyrazine-2-carboxylate |
| TDH-174 | (R)-2-((4-fluorophenyl)amino)-2-oxo-1-phenylethyl 3-amino-6-(1-(1-(2-(2-(2-(2-((3-(2,6- |
| | dioxopiperidin-3-yl)-4-oxo-3,4-dihydrobenzo[d][1,2,3]triazin-6- |
| | yl)amino)acetamido)ethoxy)ethoxy)ethyl)piperidin-4-yl)-1H-pyrazol-4-yl)pyrazine-2- |
| | carboxylate |
| TDH-175 | (R)-2-((4-fluorophenyl)amino)-2-oxo-1-phenylethyl 3-amino-6-(1-(1-(1-((3-(2,6- |
| | dioxopiperidin-3-yl)-4-oxo-3,4-dihydrobenzo[d][1,2,3]triazin-6-yl)amino)-2-oxo-6,9,12- |
| | trioxa-3-azatetradecan-14-yl)piperidin-4-yl)-1H-pyrazol-4-yl)pyrazine-2-carboxylate |
| TDH-176 | (R)-2-((4-fluorophenyl)amino)-2-oxo-1-phenylethyl 3-amino-6-(1-(1-(10-(2-((3-(2,6- |
| | dioxopiperidin-3-yl)-4-oxo-3,4-dihydrobenzo[d][1,2,3]triazin-6- |
| | yl)oxy)acetamido)decyl)piperidin-4-yl)-1H-pyrazol-4-yl)pyrazine-2-carboxylate |
| TDH-177 | (R)-2-((4-fluorophenyl)amino)-2-oxo-1-phenylethyl 3-amino-6-(1-(1-(10-(1-(3-(2,6- |
| | dioxopiperidin-3-yl)-4-oxo-3,4-dihydrobenzo[d][1,2,3]triazin-6-yl)piperidine-4- |
| | carboxamido)decyl)piperidin-4-yl)-1H-pyrazol-4-yl)pyrazine-2-carboxylate |
| TDH-178 | (R)-2-((4-fluorophenyl)amino)-2-oxo-1-phenylethyl 3-amino-6-(1-(1-(10-(1-(3-(2,6- |
| | dioxopiperidin-3-yl)-4-oxo-3,4-dihydrobenzo[d][1,2,3]triazin-6-yl)azetidine-3- |
| | carboxamido)decyl)piperidin-4-yl)-1H-pyrazol-4-yl)pyrazine-2-carboxylate |
| TDH-179 | (R)-2-((4-fluorophenyl)amino)-2-oxo-1-phenylethyl 3-amino-6-(1-(1-(10-((3-(2,6- |
| | dioxopiperidin-3-yl)-4-oxo-3,4-dihydrobenzo[d][1,2,3]triazin-6-yl)amino)decyl)piperidin-4- |
| | yl)-1H-pyrazol-4-yl)pyrazine-2-carboxylate |
| TDH-186 | 3-amino-6-(1-(1-((1-(2-((2-(2,6-dioxopiperidin-3-yl)-1,3-dioxoisoindolin-4- |
| | yl)oxy)acetyl)piperidin-4-yl)methyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-187 | 3-amino-6-(1-(1-((1-(2-((2-(2,6-dioxopiperidin-3-yl)-1,3-dioxoisoindolin-5- |
| | yl)oxy)acetyl)piperidin-4-yl)methyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-188 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-4-yl)oxy)acetamido)ethoxy)ethoxy)acetyl)piperidin- |
| | 4-yl)-1H-pyrazol-4-yl)pyrazine-2-carboxylate |
| TDH-189 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-5-yl)oxy)acetamido)ethoxy)ethoxy)acetyl)piperidin- |
| | 4-yl)-1H-pyrazol-4-yl)pyrazine-2-carboxylate |
| TDH-190 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-4- |
| | yl)amino)acetamido)ethoxy)ethoxy)acetyl)piperidin-4-yl)-1H-pyrazol-4-yl)pyrazine-2- |
| | carboxylate |
| TDH-191 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-5- |
| | yl)amino)acetamido)ethoxy)ethoxy)acetyl)piperidin-4-yl)-1H-pyrazol-4-yl)pyrazine-2- |
| | carboxylate |
| TDH-192 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-4-yl)azetidine-3- |
| | carboxamido)ethoxy)ethoxy)acetyl)piperidin-4-yl)-1H-pyrazol-4-yl)pyrazine-2-carboxylate |
| TDH-193 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-5-yl)azetidine-3- |
| | carboxamido)ethoxy)ethoxy)acetyl)piperidin-4-yl)-1H-pyrazol-4-yl)pyrazine-2-carboxylate |
| TDH-194 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-4-yl)piperidine-4- |
| | carboxamido)ethoxy)ethoxy)acetyl)piperidin-4-yl)-1H-pyrazol-4-yl)pyrazine-2-carboxylate |
| TDH-195 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-5-yl)piperidine-4- |
| | carboxamido)ethoxy)ethoxy)acetyl)piperidin-4-yl)-1H-pyrazol-4-yl)pyrazine-2-carboxylate |
| TDH-196 | (R)-2-((4-fluorophenyl)amino)-2-oxo-1-phenylethyl 3-amino-6-(1-(1-(2-(1-(1-(2-(2,6- |
| | dioxopiperidin-3-yl)-1,3-dioxoisoindolin-4-yl)piperidine-4-carbonyl)piperidin-4- |
| | yl)ethyl)piperidin-4-yl)-1H-pyrazol-4-yl)pyrazine-2-carboxylate |
| TDH-197 | (R)-2-((4-fluorophenyl)amino)-2-oxo-1-phenylethyl 3-amino-6-(1-(1-(2-(1-(1-(2-(2,6- |
| | dioxopiperidin-3-yl)-1,3-dioxoisoindolin-5-yl)piperidine-4-carbonyl)piperidin-4- |
| | yl)ethyl)piperidin-4-yl)-1H-pyrazol-4-yl)pyrazine-2-carboxylate |
| TDH-198 | (R)-2-((4-fluorophenyl)amino)-2-oxo-1-phenylethyl 3-amino-6-(1-(1-(2-(1-((2-(2,6- |
| | dioxopiperidin-3-yl)-1,3-dioxoisoindolin-4-yl)glycyl)piperidin-4-yl)ethyl)piperidin-4-yl)-1H- |
| | pyrazol-4-yl)pyrazine-2-carboxylate |
| TDH-199 | (R)-2-((4-fluorophenyl)amino)-2-oxo-1-phenylethyl 3-amino-6-(1-(1-(2-(1-((2-(2,6- |
| | dioxopiperidin-3-yl)-1,3-dioxoisoindolin-5-yl)glycyl)piperidin-4-yl)ethyl)piperidin-4-yl)-1H- |
| | pyrazol-4-yl)pyrazine-2-carboxylate |
| TDH-200 | (R)-2-((4-fluorophenyl)amino)-2-oxo-1-phenylethyl 3-amino-6-(1-(1-(2-(1-(2-((2-(2,6- |
| | dioxopiperidin-3-yl)-1,3-dioxoisoindolin-4-yl)oxy)acetyl)piperidin-4-yl)ethyl)piperidin-4-yl)- |
| | 1H-pyrazol-4-yl)pyrazine-2-carboxylate |
| TDH-201 | (R)-2-((4-fluorophenyl)amino)-2-oxo-1-phenylethyl 3-amino-6-(1-(1-(2-(1-(2-((2-(2,6- |
| | dioxopiperidin-3-yl)-1,3-dioxoisoindolin-5-yl)oxy)acetyl)piperidin-4-yl)ethyl)piperidin-4-yl)- |
| | 1H-pyrazol-4-yl)pyrazine-2-carboxylate |
| TDH-202 | (R)-2-((4-fluorophenyl)amino)-2-oxo-1-phenylethyl 3-amino-6-(1-(1-(2-(1-(2-(2,6- |
| | dioxopiperidin-3-yl)-1,3-dioxoisoindolin-4-yl)piperidin-4-yl)ethyl)piperidin-4-yl)-1H-pyrazol- |
| | 4-yl)pyrazine-2-carboxylate |
| TDH-203 | (R)-2-((4-fluorophenyl)amino)-2-oxo-1-phenylethyl 3-amino-6-(1-(1-(2-(1-(2-(2,6- |
| | dioxopiperidin-3-yl)-1,3-dioxoisoindolin-5-yl)piperidin-4-yl)ethyl)piperidin-4-yl)-1H-pyrazol- |
| | 4-yl)pyrazine-2-carboxylate |
| TDH-204 | (R)-2-((4-fluorophenyl)amino)-2-oxo-1-phenylethyl 3-amino-6-(1-(1-((1-(2-(1-(2-(2,6- |
| | dioxopiperidin-3-yl)-1,3-dioxoisoindolin-4-yl)piperidin-4-yl)acetyl)piperidin-4- |
| | yl)methyl)piperidin-4-yl)-1H-pyrazol-4-yl)pyrazine-2-carboxylate |
| TDH-205 | (R)-2-((4-fluorophenyl)amino)-2-oxo-1-phenylethyl 3-amino-6-(1-(1-((1-(2-(1-(2-(2,6- |
| | dioxopiperidin-3-yl)-1,3-dioxoisoindolin-5-yl)piperidin-4-yl)acetyl)piperidin-4- |
| | yl)methyl)piperidin-4-yl)-1H-pyrazol-4-yl)pyrazine-2-carboxylate |
| TDH-206 | 3-amino-6-(1-(1-((1-((2-(2,6-dioxopiperidin-3-yl)-1,3-dioxoisoindolin-4-yl)glycyl)piperidin-4- |
| | yl)methyl)piperidin-4-yl)-1H-pyrazol-4-yl)-N-((R)-2-((4-fluorophenyl)amino)-2-oxo-1- |
| | phenylethyl)pyrazine-2-carboxamide |
| TDH-207 | 3-amino-6-(1-(1-((1-((2-(2,6-dioxopiperidin-3-yl)-1,3-dioxoisoindolin-5-yl)glycyl)piperidin-4- |
| | yl)methyl)piperidin-4-yl)-1H-pyrazol-4-yl)-N-((R)-2-((4-fluorophenyl)amino)-2-oxo-1- |
| | phenylethyl)pyrazine-2-carboxamide |
| TDH-208 | 3-amino-6-(1-(1-((1-(2-(2,6-dioxopiperidin-3-yl)-1,3-dioxoisoindolin-4-yl)piperidin-4- |
| | yl)methyl)piperidin-4-yl)-1H-pyrazol-4-yl)-N-((R)-2-((4-fluorophenyl)amino)-2-oxo-1- |
| | phenylethyl)pyrazine-2-carboxamide |
| TDH-209 | 3-amino-6-(1-(1-((1-(1-(2-(2,6-dioxopiperidin-3-yl)-1,3-dioxoisoindolin-4-yl)piperidine-4- |
| | carbonyl)piperidin-4-yl)methyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-210 | 3-amino-6-(1-(1-((1-(1-(2-(2,6-dioxopiperidin-3-yl)-1,3-dioxoisoindolin-5-yl)piperidine-4- |
| | carbonyl)piperidin-4-yl)methyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-211 | (R)-2-((4-fluorophenyl)amino)-2-oxo-1-phenylethyl 3-amino-6-(1-(1-((1-(2-(((S)-1-((2S,4R)- |
| | 4-hydroxy-2-((4-(4-methylthiazol-5-yl)benzyl)carbamoyl)pyrrolidin-1-yl)-3,3-dimethyl-1- |
| | oxobutan-2-yl)amino)-2-oxoethyl)piperidin-4-yl)methyl)piperidin-4-yl)-1H-pyrazol-4- |
| | yl)pyrazine-2-carboxylate |
| TDH-212 | (R)-2-((4-fluorophenyl)amino)-2-oxo-1-phenylethyl 3-amino-6-(1-(1-((1-(4-(((S)-1-((2S,4R)- |
| | 4-hydroxy-2-((4-(4-methylthiazol-5-yl)benzyl)carbamoyl)pyrrolidin-1-yl)-3,3-dimethyl-1- |
| | oxobutan-2-yl)amino)-4-oxobutanoyl)piperidin-4-yl)methyl)piperidin-4-yl)-1H-pyrazol-4- |
| | yl)pyrazine-2-carboxylate |
| TDH-213 | 3-amino-6-(1-(1-(2-(2-(2-(1-(2-(2,6-dioxopiperidin-3-yl)-1,3-dioxoisoindolin-4-yl)piperidine- |
| | 4-carboxamido)ethoxy)ethoxy)ethyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-214 | 3-amino-6-(1-(1-(2-(2-(2-(1-(2-(2,6-dioxopiperidin-3-yl)-1,3-dioxoisoindolin-5-yl)piperidine- |
| | 4-carboxamido)ethoxy)ethoxy)ethyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-215 | 3-amino-6-(1-(1-(2-(2-(2-((2-(2,6-dioxopiperidin-3-yl)-1,3-dioxoisoindolin-4- |
| | yl)amino)ethoxy)ethoxy)ethyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-216 | 3-amino-6-(1-(1-(2-(2-(2-((2-(2,6-dioxopiperidin-3-yl)-1,3-dioxoisoindolin-5- |
| | yl)amino)ethoxy)ethoxy)ethyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-217 | 3-amino-6-(1-(1-(2-(2-(2-(2-((2-(2,6-dioxopiperidin-3-yl)-1,3-dioxoisoindolin-4- |
| | yl)amino)acetamido)ethoxy)ethoxy)ethyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-218 | 3-amino-6-(1-(1-(2-(2-(2-(2-((2-(2,6-dioxopiperidin-3-yl)-1,3-dioxoisoindolin-5- |
| | yl)amino)acetamido)ethoxy)ethoxy)ethyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-221 | 2-((4-fluorophenyl)amino)-2-oxo-1-phenylethyl 3-amino-6-(1-(1-((1-(1-(2-(2,6- |
| | dioxopiperidin-3-yl)-1,3-dioxoisoindolin-5-yl)piperidine-4-carbonyl)piperidin-4- |
| | yl)methyl)piperidin-4-yl)-1H-1,2,3-triazol-4-yl)pyrazine-2-carboxylate |
| TDH-222 | 2-((4-fluorophenyl)amino)-2-oxo-1-phenylethyl 3-amino-6-(1-(1-((1-(1-(2-(2,6- |
| | dioxopiperidin-3-yl)-1,3-dioxoisoindolin-4-yl)piperidine-4-carbonyl)piperidin-4- |
| | yl)methyl)piperidin-4-yl)-1H-1,2,3-triazol-4-yl)pyrazine-2-carboxylate |
| TDH-223 | 2-((4-fluorophenyl)amino)-2-oxo-1-phenylethyl 3-amino-6-(1-(1-((1-((2-(2,6-dioxopiperidin- |
| | 3-yl)-1,3-dioxoisoindolin-5-yl)glycyl)piperidin-4-yl)methyl)piperidin-4-yl)-1H-1,2,3-triazol-4- |
| | yl)pyrazine-2-carboxylate |
| TDH-224 | 2-((4-fluorophenyl)amino)-2-oxo-1-phenylethyl 3-amino-6-(1-(1-((1-((2-(2,6-dioxopiperidin- |
| | 3-yl)-1,3-dioxoisoindolin-4-yl)glycyl)piperidin-4-yl)methyl)piperidin-4-yl)-1H-1,2,3-triazol-4- |
| | yl)pyrazine-2-carboxylate |
| TDH-225 | 2-((4-fluorophenyl)amino)-2-oxo-1-phenylethyl 3-amino-6-(1-(1-((1-(2-(2,6-dioxopiperidin- |
| | 3-yl)-1,3-dioxoisoindolin-4-yl)piperidin-4-yl)methyl)piperidin-4-yl)-1H-1,2,3-triazol-4- |
| | yl)pyrazine-2-carboxylate |
| TDH-226 | 2-((4-fluorophenyl)amino)-2-oxo-1-phenylethyl 3-amino-6-(1-(1-((1-(2-(2,6-dioxopiperidin- |
| | 3-yl)-1,3-dioxoisoindolin-5-yl)piperidin-4-yl)methyl)piperidin-4-yl)-1H-1,2,3-triazol-4- |
| | yl)pyrazine-2-carboxylate |
| TDH-230 | 2-((4-fluorophenyl)amino)-2-oxo-1-phenylethyl 3-amino-6-(6-(4-((1-(1-(2-(2,6- |
| | dioxopiperidin-3-yl)-1,3-dioxoisoindolin-4-yl)piperidine-4-carbonyl)piperidin-4- |
| | yl)methyl)piperazin-1-yl)pyridin-3-yl)pyrazine-2-carboxylate |
| TDH-231 | 2-((4-fluorophenyl)amino)-2-oxo-1-phenylethyl 3-amino-6-(6-(4-((1-(1-(2-(2,6- |
| | dioxopiperidin-3-yl)-1,3-dioxoisoindolin-5-yl)piperidine-4-carbonyl)piperidin-4- |
| | yl)methyl)piperazin-1-yl)pyridin-3-yl)pyrazine-2-carboxylate |
| TDH-232 | 2-((4-fluorophenyl)amino)-2-oxo-1-phenylethyl 3-amino-6-(6-(4-((1-((2-(2,6-dioxopiperidin- |
| | 3-yl)-1,3-dioxoisoindolin-4-yl)glycyl)piperidin-4-yl)methyl)piperazin-1-yl)pyridin-3- |
| | yl)pyrazine-2-carboxylate |
| TDH-233 | 2-((4-fluorophenyl)amino)-2-oxo-1-phenylethyl 3-amino-6-(6-(4-((1-((2-(2,6-dioxopiperidin- |
| | 3-yl)-1,3-dioxoisoindolin-5-yl)glycyl)piperidin-4-yl)methyl)piperazin-1-yl)pyridin-3- |
| | yl)pyrazine-2-carboxylate |
| TDH-234 | 2-((4-fluorophenyl)amino)-2-oxo-1-phenylethyl 3-amino-6-(1-(1-((1-(2-((2-((2-(2,6- |
| | dioxopiperidin-3-yl)-1,3-dioxoisoindolin-4-yl)amino)ethyl)amino)-2-oxoethyl)piperidin-4- |
| | yl)methyl)piperidin-4-yl)-1H-pyrazol-4-yl)pyrazine-2-carboxylate |
| TDH-235 | 2-((4-fluorophenyl)amino)-2-oxo-1-phenylethyl 3-amino-6-(1-(1-((1-(2-((3-((2-(2,6- |
| | dioxopiperidin-3-yl)-1,3-dioxoisoindolin-4-yl)amino)propyl)amino)-2-oxoethyl)piperidin-4- |
| | yl)methyl)piperidin-4-yl)-1H-pyrazol-4-yl)pyrazine-2-carboxylate |
| TDH-236 | 2-((4-fluorophenyl)amino)-2-oxo-1-phenylethyl 3-amino-6-(1-(1-((1-(2-((1-(2-(2,6- |
| | dioxopiperidin-3-yl)-1,3-dioxoisoindolin-4-yl)piperidin-4-yl)amino)-2-oxoethyl)piperidin-4- |
| | yl)methyl)piperidin-4-yl)-1H-pyrazol-4-yl)pyrazine-2-carboxylate |
| TDH-237 | 2-((4-fluorophenyl)amino)-2-oxo-1-phenylethyl 3-amino-6-(1-(1-((1-(2-(4-(2-(2,6- |
| | dioxopiperidin-3-yl)-1,3-dioxoisoindolin-4-yl)piperazin-1-yl)-2-oxoethyl)piperidin-4- |
| | yl)methyl)piperidin-4-yl)-1H-pyrazol-4-yl)pyrazine-2-carboxylate |
| TDH-238 | 2-((4-fluorophenyl)amino)-2-oxo-1-phenylethyl 3-amino-6-(1-(1-((1-(2-(4-((2-(2,6- |
| | dioxopiperidin-3-yl)-1,3-dioxoisoindolin-4-yl)amino)piperidin-1-yl)-2-oxoethyl)piperidin-4- |
| | yl)methyl)piperidin-4-yl)-1H-pyrazol-4-yl)pyrazine-2-carboxylate |
| TDH-239 | 2-((4-fluorophenyl)amino)-2-oxo-1-phenylethyl 3-amino-6-(1-(1-(3-(4-(1-(2-(2,6- |
| | dioxopiperidin-3-yl)-1,3-dioxoisoindolin-4-yl)piperidine-4-carbonyl)piperazin-1- |
| | yl)propyl)piperidin-4-yl)-1H-pyrazol-4-yl)pyrazine-2-carboxylate |
| TDH-240 | 2-((4-fluorophenyl)amino)-2-oxo-1-phenylethyl 3-amino-6-(1-(1-(3-(4-(1-(2-(2,6- |
| | dioxopiperidin-3-yl)-1,3-dioxoisoindolin-5-yl)piperidine-4-carbonyl)piperazin-1- |
| | yl)propyl)piperidin-4-yl)-1H-pyrazol-4-yl)pyrazine-2-carboxylate |
| TDH-241 | 2-((4-fluorophenyl)amino)-2-oxo-1-phenylethyl 3-amino-6-(1-(1-(3-(4-((2-(2,6- |
| | dioxopiperidin-3-yl)-1,3-dioxoisoindolin-4-yl)glycyl)piperazin-1-yl)propyl)piperidin-4-yl)-1H- |
| | pyrazol-4-yl)pyrazine-2-carboxylate |
| TDH-242 | 2-((4-fluorophenyl)amino)-2-oxo-1-phenylethyl 3-amino-6-(1-(1-(3-(4-((2-(2,6- |
| | dioxopiperidin-3-yl)-1,3-dioxoisoindolin-5-yl)glycyl)piperazin-1-yl)propyl)piperidin-4-yl)-1H- |
| | pyrazol-4-yl)pyrazine-2-carboxylate |
| TDH-243 | 2-((4-fluorophenyl)amino)-2-oxo-1-phenylethyl 3-amino-6-(1-(1-(3-(4-(2-((2-(2,6- |
| | dioxopiperidin-3-yl)-1,3-dioxoisoindolin-4-yl)oxy)acetyl)piperazin-1-yl)propyl)piperidin-4- |
| | yl)-1H-pyrazol-4-yl)pyrazine-2-carboxylate |
| TDH-244 | 2-((4-fluorophenyl)amino)-2-oxo-1-phenylethyl 3-amino-6-(1-(1-(3-(4-(2-((2-(2,6- |
| | dioxopiperidin-3-yl)-1,3-dioxoisoindolin-5-yl)oxy)acetyl)piperazin-1-yl)propyl)piperidin-4- |
| | yl)-1H-pyrazol-4-yl)pyrazine-2-carboxylate |
| TDH-245 | 2-((4-fluorophenyl)amino)-2-oxo-1-phenylethyl 3-amino-6-(1-(1-(3-(4-(2-(2,6- |
| | dioxopiperidin-3-yl)-1,3-dioxoisoindolin-4-yl)piperazin-1-yl)propyl)piperidin-4-yl)-1H- |
| | pyrazol-4-yl)pyrazine-2-carboxylate |
| TDH-246 | 2-((4-fluorophenyl)amino)-2-oxo-1-phenylethyl 3-amino-6-(1-(1-(3-(4-(2-(2,6- |
| | dioxopiperidin-3-yl)-1,3-dioxoisoindolin-5-yl)piperazin-1-yl)propyl)piperidin-4-yl)-1H- |
| | pyrazol-4-yl)pyrazine-2-carboxylate |
| TDH-247 | 2-((4-fluorophenyl)amino)-2-oxo-1-phenylethyl 3-amino-6-(1-(1-(2-((1-(1-(2-(2,6- |
| | dioxopiperidin-3-yl)-1,3-dioxoisoindolin-4-yl)piperidine-4-carbonyl)piperidin-4- |
| | yl)oxy)ethyl)piperidin-4-yl)-1H-pyrazol-4-yl)pyrazine-2-carboxylate |
| TDH-248 | 2-((4-fluorophenyl)amino)-2-oxo-1-phenylethyl 3-amino-6-(1-(1-(2-((1-(1-(2-(2,6- |
| | dioxopiperidin-3-yl)-1,3-dioxoisoindolin-5-yl)piperidine-4-carbonyl)piperidin-4- |
| | yl)oxy)ethyl)piperidin-4-yl)-1H-pyrazol-4-yl)pyrazine-2-carboxylate |
| TDH-249 | 2-((4-fluorophenyl)amino)-2-oxo-1-phenylethyl 3-amino-6-(1-(1-(2-((1-((2-(2,6- |
| | dioxopiperidin-3-yl)-1,3-dioxoisoindolin-4-yl)glycyl)piperidin-4-yl)oxy)ethyl)piperidin-4-yl)- |
| | 1H-pyrazol-4-yl)pyrazine-2-carboxylate |
| TDH-250 | 2-((4-fluorophenyl)amino)-2-oxo-1-phenylethyl 3-amino-6-(1-(1-(2-((1-((2-(2,6- |
| | dioxopiperidin-3-yl)-1,3-dioxoisoindolin-5-yl)glycyl)piperidin-4-yl)oxy)ethyl)piperidin-4-yl)- |
| | 1H-pyrazol-4-yl)pyrazine-2-carboxylate |
| TDH-251 | 2-((4-fluorophenyl)amino)-2-oxo-1-phenylethyl 3-amino-6-(1-(1-(2-((1-(2-((2-(2,6- |
| | dioxopiperidin-3-yl)-1,3-dioxoisoindolin-4-yl)oxy)acetyl)piperidin-4-yl)oxy)ethyl)piperidin-4- |
| | yl)-1H-pyrazol-4-yl)pyrazine-2-carboxylate |
| TDH-252 | 2-((4-fluorophenyl)amino)-2-oxo-1-phenylethyl 3-amino-6-(1-(1-(2-((1-(2-((2-(2,6- |
| | dioxopiperidin-3-yl)-1,3-dioxoisoindolin-5-yl)oxy)acetyl)piperidin-4-yl)oxy)ethyl)piperidin-4- |
| | yl)-1H-pyrazol-4-yl)pyrazine-2-carboxylate |
| TDH-253 | 2-((4-fluorophenyl)amino)-2-oxo-1-phenylethyl 3-amino-6-(1-(1-(2-((1-(2-(2,6- |
| | dioxopiperidin-3-yl)-1,3-dioxoisoindolin-4-yl)piperidin-4-yl)oxy)ethyl)piperidin-4-yl)-1H- |
| | pyrazol-4-yl)pyrazine-2-carboxylate |
| TDH-254 | 2-((4-fluorophenyl)amino)-2-oxo-1-phenylethyl 3-amino-6-(1-(1-(2-((1-(2-(2,6- |
| | dioxopiperidin-3-yl)-1,3-dioxoisoindolin-5-yl)piperidin-4-yl)oxy)ethyl)piperidin-4-yl)-1H- |
| | pyrazol-4-yl)pyrazine-2-carboxylate |
| TDH-255 | 2-((4-fluorophenyl)amino)-2-oxo-1-phenylethyl 3-amino-6-(6-(4-((1-(2-((2-(2,6- |
| | dioxopiperidin-3-yl)-1,3-dioxoisoindolin-4-yl)oxy)acetyl)piperidin-4-yl)methyl)piperazin-1- |
| | yl)pyridin-3-yl)pyrazine-2-carboxylate |
| TDH-256 | 2-((4-fluorophenyl)amino)-2-oxo-1-phenylethyl 3-amino-6-(6-(4-((1-(2-((2-(2,6- |
| | dioxopiperidin-3-yl)-1,3-dioxoisoindolin-5-yl)oxy)acetyl)piperidin-4-yl)methyl)piperazin-1- |
| | yl)pyridin-3-yl)pyrazine-2-carboxylate |
| TDH-257 | 2-((4-fluorophenyl)amino)-2-oxo-1-phenylethyl 3-amino-6-(6-(4-(2-(2-(2-(1-(2-(2,6- |
| | dioxopiperidin-3-yl)-1,3-dioxoisoindolin-4-yl)piperidine-4- |
| | carboxamido)ethoxy)ethoxy)ethyl)piperazin-1-yl)pyridin-3-yl)pyrazine-2-carboxylate |
| TDH-258 | 2-((4-fluorophenyl)amino)-2-oxo-1-phenylethyl 3-amino-6-(6-(4-(2-(2-(2-(1-(2-(2,6- |
| | dioxopiperidin-3-yl)-1,3-dioxoisoindolin-5-yl)piperidine-4- |
| | carboxamido)ethoxy)ethoxy)ethyl)piperazin-1-yl)pyridin-3-yl)pyrazine-2-carboxylate |
| TDH-259 | 2-((4-fluorophenyl)amino)-2-oxo-1-phenylethyl 3-amino-6-(6-(4-(2-(2-(2-(2-((2-(2,6- |
| | dioxopiperidin-3-yl)-1,3-dioxoisoindolin-4- |
| | yl)amino)acetamido)ethoxy)ethoxy)ethyl)piperazin-1-yl)pyridin-3-yl)pyrazine-2-carboxylate |
| TDH-260 | 2-((4-fluorophenyl)amino)-2-oxo-1-phenylethyl 3-amino-6-(6-(4-(2-(2-(2-(2-((2-(2,6- |
| | dioxopiperidin-3-yl)-1,3-dioxoisoindolin-5- |
| | yl)amino)acetamido)ethoxy)ethoxy)ethyl)piperazin-1-yl)pyridin-3-yl)pyrazine-2-carboxylate |
| TDH-261 | 2-((4-fluorophenyl)amino)-2-oxo-1-phenylethyl 3-amino-6-(6-(4-(2-(2-(2-(2-((2-(2,6- |
| | dioxopiperidin-3-yl)-1,3-dioxoisoindolin-4-yl)oxy)acetamido)ethoxy)ethoxy)ethyl)piperazin- |
| | 1-yl)pyridin-3-yl)pyrazine-2-carboxylate |
| TDH-262 | 2-((4-fluorophenyl)amino)-2-oxo-1-phenylethyl 3-amino-6-(6-(4-(2-(2-(2-(2-((2-(2,6- |
| | dioxopiperidin-3-yl)-1,3-dioxoisoindolin-5-yl)oxy)acetamido)ethoxy)ethoxy)ethyl)piperazin- |
| | 1-yl)pyridin-3-yl)pyrazine-2-carboxylate |
| TDH-263 | 2-((4-fluorophenyl)amino)-2-oxo-1-phenylethyl 3-amino-6-(1-((3S)-1-(2-(2-(2-(2-((2-(2,6- |
| | dioxopiperidin-3-yl)-1,3-dioxoisoindolin-4- |
| | yl)amino)acetamido)ethoxy)ethoxy)ethyl)pyrrolidin-3-yl)-1H-pyrazol-4-yl)pyrazine-2- |
| | carboxylate |
| TDH-264 | 2-((4-fluorophenyl)amino)-2-oxo-1-phenylethyl 3-amino-6-(1-((3S)-1-(2-(2-(2-(2-((2-(2,6- |
| | dioxopiperidin-3-yl)-1,3-dioxoisoindolin-5- |
| | yl)amino)acetamido)ethoxy)ethoxy)ethyl)pyrrolidin-3-yl)-1H-pyrazol-4-yl)pyrazine-2- |
| | carboxylate |
| TDH-265 | 2-((4-fluorophenyl)amino)-2-oxo-1-phenylethyl 3-amino-6-(1-((3S)-1-(2-(2-(2-(2-((2-(2,6- |
| | dioxopiperidin-3-yl)-1,3-dioxoisoindolin-4-yl)oxy)acetamido)ethoxy)ethoxy)ethyl)pyrrolidin- |
| | 3-yl)-1H-pyrazol-4-yl)pyrazine-2-carboxylate |
| TDH-266 | 2-((4-fluorophenyl)amino)-2-oxo-1-phenylethyl 3-amino-6-(1-((3S)-1-(2-(2-(2-(2-((2-(2,6- |
| | dioxopiperidin-3-yl)-1,3-dioxoisoindolin-5-yl)oxy)acetamido)ethoxy)ethoxy)ethyl)pyrrolidin- |
| | 3-yl)-1H-pyrazol-4-yl)pyrazine-2-carboxylate |
| TDH-267 | 2-((4-fluorophenyl)amino)-2-oxo-1-phenylethyl 3-amino-6-(1-((3R)-1-(2-(2-(2-(2-((2-(2,6- |
| | dioxopiperidin-3-yl)-1,3-dioxoisoindolin-4- |
| | yl)amino)acetamido)ethoxy)ethoxy)ethyl)pyrrolidin-3-yl)-1H-pyrazol-4-yl)pyrazine-2- |
| | carboxylate |
| TDH-268 | 2-((4-fluorophenyl)amino)-2-oxo-1-phenylethyl 3-amino-6-(1-((3R)-1-(2-(2-(2-(2-((2-(2,6- |
| | dioxopiperidin-3-yl)-1,3-dioxoisoindolin-5- |
| | yl)amino)acetamido)ethoxy)ethoxy)ethyl)pyrrolidin-3-yl)-1H-pyrazol-4-yl)pyrazine-2- |
| | carboxylate |
| TDH-269 | 2-((4-fluorophenyl)amino)-2-oxo-1-phenylethyl 3-amino-6-(1-((3R)-1-(2-(2-(2-(2-((2-(2,6- |
| | dioxopiperidin-3-yl)-1,3-dioxoisoindolin-4-yl)oxy)acetamido)ethoxy)ethoxy)ethyl)pyrrolidin- |
| | 3-yl)-1H-pyrazol-4-yl)pyrazine-2-carboxylate |
| TDH-270 | 2-((4-fluorophenyl)amino)-2-oxo-1-phenylethyl 3-amino-6-(1-((3R)-1-(2-(2-(2-(2-((2-(2,6- |
| | dioxopiperidin-3-yl)-1,3-dioxoisoindolin-5-yl)oxy)acetamido)ethoxy)ethoxy)ethyl)pyrrolidin- |
| | 3-yl)-1H-pyrazol-4-yl)pyrazine-2-carboxylate |
| TDH-271 | 2-((4-fluorophenyl)amino)-2-oxo-1-phenylethyl 3-amino-6-(1-(1-((1-(2-(3-((2-(2,6- |
| | dioxopiperidin-3-yl)-1,3-dioxoisoindolin-4-yl)amino)azetidin-1-yl)-2-oxoethyl)piperidin-4- |
| | yl)methyl)piperidin-4-yl)-1H-pyrazol-4-yl)pyrazine-2-carboxylate |
| TDH-272 | 2-((4-fluorophenyl)amino)-2-oxo-1-phenylethyl 3-amino-6-(1-(1-((1-(2-((1-(2-(2,6- |
| | dioxopiperidin-3-yl)-1,3-dioxoisoindolin-5-yl)piperidin-4-yl)amino)-2-oxoethyl)piperidin-4- |
| | yl)methyl)piperidin-4-yl)-1H-pyrazol-4-yl)pyrazine-2-carboxylate |
| TDH-273 | 2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-4-yl)piperidine-4-carbonyl)piperidine-4- |
| | carbonyl)piperidin-4-yl)-1H-pyrazol-4-yl)pyrazine-2-carboxylate |
| TDH-274 | 2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-5-yl)piperidine-4-carbonyl)piperidine-4- |
| | carbonyl)piperidin-4-yl)-1H-pyrazol-4-yl)pyrazine-2-carboxylate |
| TDH-275 | 2-((4-fluorophenyl)amino)-2-oxo-1-phenylethyl 3-amino-6-(1-(1-(1-((2-(2,6-dioxopiperidin- |
| | 3-yl)-1,3-dioxoisoindolin-4-yl)glycyl)piperidine-4-carbonyl)piperidin-4-yl)-1H-pyrazol-4- |
| | yl)pyrazine-2-carboxylate |
| TDH-276 | 2-((4-fluorophenyl)amino)-2-oxo-1-phenylethyl 3-amino-6-(1-(1-(1-((2-(2,6-dioxopiperidin- |
| | 3-yl)-1,3-dioxoisoindolin-5-yl)glycyl)piperidine-4-carbonyl)piperidin-4-yl)-1H-pyrazol-4- |
| | yl)pyrazine-2-carboxylate |
| TDH-277 | 2-((4-fluorophenyl)amino)-2-oxo-1-phenylethyl 3-amino-6-(1-(1-(1-(2-((2-(2,6- |
| | dioxopiperidin-3-yl)-1,3-dioxoisoindolin-4-yl)oxy)acetyl)piperidine-4-carbonyl)piperidin-4- |
| | yl)-1H-pyrazol-4-yl)pyrazine-2-carboxylate |
| TDH-278 | 2-((4-fluorophenyl)amino)-2-oxo-1-phenylethyl 3-amino-6-(1-(1-(1-(2-((2-(2,6- |
| | dioxopiperidin-3-yl)-1,3-dioxoisoindolin-5-yl)oxy)acetyl)piperidine-4-carbonyl)piperidin-4- |
| | yl)-1H-pyrazol-4-yl)pyrazine-2-carboxylate |
| TDH-279 | 2-((4-fluorophenyl)amino)-2-oxo-1-phenylethyl 3-amino-6-(1-(1-(1-(2-(2,6-dioxopiperidin-3- |
| | yl)-1,3-dioxoisoindolin-4-yl)piperidine-4-carbonyl)piperidin-4-yl)-1H-pyrazol-4-yl)pyrazine- |
| | 2-carboxylate |
| TDH-280 | 2-((4-fluorophenyl)amino)-2-oxo-1-phenylethyl 3-amino-6-(1-(1-(1-(2-(2,6-dioxopiperidin-3- |
| | yl)-1,3-dioxoisoindolin-5-yl)piperidine-4-carbonyl)piperidin-4-yl)-1H-pyrazol-4-yl)pyrazine- |
| | 2-carboxylate |
| TDH-281 | 2-((4-fluorophenyl)amino)-2-oxo-1-phenylethyl 3-amino-6-(1-((3S)-1-((1-((2-(2,6- |
| | dioxopiperidin-3-yl)-1,3-dioxoisoindolin-4-yl)glycyl)piperidin-4-yl)methyl)pyrrolidin-3-yl)-1H- |
| | pyrazol-4-yl)pyrazine-2-carboxylate |
| TDH-282 | 2-((4-fluorophenyl)amino)-2-oxo-1-phenylethyl 3-amino-6-(1-((3S)-1-((1-((2-(2,6- |
| | dioxopiperidin-3-yl)-1,3-dioxoisoindolin-5-yl)glycyl)piperidin-4-yl)methyl)pyrrolidin-3-yl)-1H- |
| | pyrazol-4-yl)pyrazine-2-carboxylate |
| TDH-283 | 2-((4-fluorophenyl)amino)-2-oxo-1-phenylethyl 3-amino-6-(1-((3S)-1-((1-(2-((2-(2,6- |
| | dioxopiperidin-3-yl)-1,3-dioxoisoindolin-4-yl)oxy)acetyl)piperidin-4-yl)methyl)pyrrolidin-3- |
| | yl)-1H-pyrazol-4-yl)pyrazine-2-carboxylate |
| TDH-284 | 2-((4-fluorophenyl)amino)-2-oxo-1-phenylethyl 3-amino-6-(1-((3S)-1-((1-(2-((2-(2,6- |
| | dioxopiperidin-3-yl)-1,3-dioxoisoindolin-5-yl)oxy)acetyl)piperidin-4-yl)methyl)pyrrolidin-3- |
| | yl)-1H-pyrazol-4-yl)pyrazine-2-carboxylate |
| TDH-285 | 2-((4-fluorophenyl)amino)-2-oxo-1-phenylethyl 3-amino-6-(1-((3R)-1-((1-((2-(2,6- |
| | dioxopiperidin-3-yl)-1,3-dioxoisoindolin-4-yl)glycyl)piperidin-4-yl)methyl)pyrrolidin-3-yl)-1H- |
| | pyrazol-4-yl)pyrazine-2-carboxylate |
| TDH-286 | 2-((4-fluorophenyl)amino)-2-oxo-1-phenylethyl 3-amino-6-(1-((3R)-1-((1-((2-(2,6- |
| | dioxopiperidin-3-yl)-1,3-dioxoisoindolin-5-yl)glycyl)piperidin-4-yl)methyl)pyrrolidin-3-yl)-1H- |
| | pyrazol-4-yl)pyrazine-2-carboxylate |
| TDH-287 | 2-((4-fluorophenyl)amino)-2-oxo-1-phenylethyl 3-amino-6-(1-((3R)-1-((1-(2-((2-(2,6- |
| | dioxopiperidin-3-yl)-1,3-dioxoisoindolin-4-yl)oxy)acetyl)piperidin-4-yl)methyl)pyrrolidin-3- |
| | yl)-1H-pyrazol-4-yl)pyrazine-2-carboxylate |
| TDH-288 | 2-((4-fluorophenyl)amino)-2-oxo-1-phenylethyl 3-amino-6-(1-((3R)-1-((1-(2-((2-(2,6- |
| | dioxopiperidin-3-yl)-1,3-dioxoisoindolin-5-yl)oxy)acetyl)piperidin-4-yl)methyl)pyrrolidin-3- |
| | yl)-1H-pyrazol-4-yl)pyrazine-2-carboxylate |
| TDH-289 | 2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-5-yl)piperidine-4- |
| | carbonyl)piperidin-4-yl)ethyl)piperidin-4-yl)-1H-pyrazol-4-yl)pyrazine-2-carboxylate |
| TDH-290 | 2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)ethyl)piperidin-4-yl)-1H-pyrazol-4-yl)pyrazine-2-carboxylate |
| TDH-291 | 2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)ethyl)piperidin-4-yl)-1H-pyrazol-4-yl)pyrazine-2-carboxylate |
| TDH-292 | 2-((4-fluorophenyl)amino)-2-oxo-1-phenylethyl 3-amino-6-(1-(1-(2-(1-((3-(2,6- |
| | dioxopiperidin-3-yl)-4-oxo-3,4-dihydrobenzo[d][1,2,3]triazin-6-yl)glycyl)piperidin-4- |
| | yl)ethyl)piperidin-4-yl)-1H-pyrazol-4-yl)pyrazine-2-carboxylate |
| TDH-293 | 2-((4-fluorophenyl)amino)-2-oxo-1-phenylethyl 3-amino-6-(1-(1-(2-(1-(2-((3-(2,6- |
| | dioxopiperidin-3-yl)-4-oxo-3,4-dihydrobenzo[d][1,2,3]triazin-6-yl)oxy)acetyl)piperidin-4- |
| | yl)ethyl)piperidin-4-yl)-1H-pyrazol-4-yl)pyrazine-2-carboxylate |
| TDH-294 | 2-((4-fluorophenyl)amino)-2-oxo-1-phenylethyl 3-amino-6-(1-(1-(2-(1-(3-(2,6- |
| | dioxopiperidin-3-yl)-4-oxo-3,4-dihydrobenzo[d][1,2,3]triazin-5-yl)piperidin-4- |
| | yl)ethyl)piperidin-4-yl)-1H-pyrazol-4-yl)pyrazine-2-carboxylate |
| TDH-295 | 2-((4-fluorophenyl)amino)-2-oxo-1-phenylethyl 3-amino-6-(1-(1-(2-(1-(3-(2,6- |
| | dioxopiperidin-3-yl)-4-oxo-3,4-dihydrobenzo[d][1,2,3]triazin-6-yl)piperidin-4- |
| | yl)ethyl)piperidin-4-yl)-1H-pyrazol-4-yl)pyrazine-2-carboxylate |
| TDH-296 | 2-((4-fluorophenyl)amino)-2-oxo-1-phenylethyl 3-amino-6-(1-(1-((1-((2-(2,6-dioxopiperidin- |
| | 3-yl)-1,3-dioxoisoindolin-4-yl)glycyl)piperidin-4-yl)methyl)azetidin-3-yl)-1H-pyrazol-4- |
| | yl)pyrazine-2-carboxylate |
| TDH-297 | 2-((4-fluorophenyl)amino)-2-oxo-1-phenylethyl 3-amino-6-(1-(1-((1-((2-(2,6-dioxopiperidin- |
| | 3-yl)-1,3-dioxoisoindolin-5-yl)glycyl)piperidin-4-yl)methyl)azetidin-3-yl)-1H-pyrazol-4- |
| | yl)pyrazine-2-carboxylate |
| TDH-298 | 2-((4-fluorophenyl)amino)-2-oxo-1-phenylethyl 3-amino-6-(1-(1-((1-(2-((2-(2,6- |
| | dioxopiperidin-3-yl)-1,3-dioxoisoindolin-4-yl)oxy)acetyl)piperidin-4-yl)methyl)azetidin-3-yl)- |
| | 1H-pyrazol-4-yl)pyrazine-2-carboxylate |
| TDH-299 | 2-((4-fluorophenyl)amino)-2-oxo-1-phenylethyl 3-amino-6-(1-(1-((1-(2-((2-(2,6- |
| | dioxopiperidin-3-yl)-1,3-dioxoisoindolin-5-yl)oxy)acetyl)piperidin-4-yl)methyl)azetidin-3-yl)- |
| | 1H-pyrazol-4-yl)pyrazine-2-carboxylate |
| TDH-300 | 2-((4-fluorophenyl)amino)-2-oxo-1-phenylethyl 2-amino-5-(1-(1-(2-(1-(2-((2-(2,6- |
| | dioxopiperidin-3-yl)-1,3-dioxoisoindolin-5-yl)oxy)acetyl)piperidin-4-yl)ethyl)piperidin-4-yl)- |
| | 1H-pyrazol-4-yl)nicotinate |
| TDH-301 | 2-((4-fluorophenyl)amino)-2-oxo-1-phenylethyl 2-amino-5-(1-(1-(2-(1-(2-((2-(2,6- |
| | dioxopiperidin-3-yl)-1,3-dioxoisoindolin-4-yl)oxy)acetyl)piperidin-4-yl)ethyl)piperidin-4-yl)- |
| | 1H-pyrazol-4-yl)nicotinate |
| TDH-302 | 2-((4-fluorophenyl)amino)-2-oxo-1-phenylethyl 2-amino-5-(1-(1-(2-(1-((2-(2,6- |
| | dioxopiperidin-3-yl)-1,3-dioxoisoindolin-5-yl)glycyl)piperidin-4-yl)ethyl)piperidin-4-yl)-1H- |
| | pyrazol-4-yl)nicotinate |
| TDH-303 | 2-((4-fluorophenyl)amino)-2-oxo-1-phenylethyl 2-amino-5-(1-(1-(2-(1-((2-(2,6- |
| | dioxopiperidin-3-yl)-1,3-dioxoisoindolin-4-yl)glycyl)piperidin-4-yl)ethyl)piperidin-4-yl)-1H- |
| | pyrazol-4-yl)nicotinate |
| TDH-304 | 2-((4-fluorophenyl)amino)-2-oxo-1-phenylethyl 3-amino-6-(1-(1-((4-(1-(2-(2,6- |
| | dioxopiperidin-3-yl)-1,3-dioxoisoindolin-4-yl)piperidine-4-carbonyl)morpholin-2- |
| | yl)methyl)piperidin-4-yl)-1H-pyrazol-4-yl)pyrazine-2-carboxylate |
| TDH-305 | 2-((4-fluorophenyl)amino)-2-oxo-1-phenylethyl 3-amino-6-(1-(1-((4-(1-(2-(2,6- |
| | dioxopiperidin-3-yl)-1,3-dioxoisoindolin-5-yl)piperidine-4-carbonyl)morpholin-2- |
| | yl)methyl)piperidin-4-yl)-1H-pyrazol-4-yl)pyrazine-2-carboxylate |
| TDH-306 | 2-((4-fluorophenyl)amino)-2-oxo-1-phenylethyl 3-amino-6-(1-(1-((4-((2-(2,6-dioxopiperidin- |
| | 3-yl)-1,3-dioxoisoindolin-4-yl)glycyl)morpholin-2-yl)methyl)piperidin-4-yl)-1H-pyrazol-4- |
| | yl)pyrazine-2-carboxylate |
| TDH-307 | 2-((4-fluorophenyl)amino)-2-oxo-1-phenylethyl 3-amino-6-(1-(1-((4-((2-(2,6-dioxopiperidin- |
| | 3-yl)-1,3-dioxoisoindolin-5-yl)glycyl)morpholin-2-yl)methyl)piperidin-4-yl)-1H-pyrazol-4- |
| | yl)pyrazine-2-carboxylate |
| TDH-308 | 2-((4-fluorophenyl)amino)-2-oxo-1-phenylethyl 3-amino-6-(1-(1-((4-(2-((2-(2,6- |
| | dioxopiperidin-3-yl)-1,3-dioxoisoindolin-4-yl)oxy)acetyl)morpholin-2-yl)methyl)piperidin-4- |
| | yl)-1H-pyrazol-4-yl)pyrazine-2-carboxylate |
| TDH-309 | 2-((4-fluorophenyl)amino)-2-oxo-1-phenylethyl 3-amino-6-(1-(1-((4-(2-((2-(2,6- |
| | dioxopiperidin-3-yl)-1,3-dioxoisoindolin-5-yl)oxy)acetyl)morpholin-2-yl)methyl)piperidin-4- |
| | yl)-1H-pyrazol-4-yl)pyrazine-2-carboxylate |
| TDH-310 | 2-((4-fluorophenyl)amino)-2-oxo-1-phenylethyl 3-amino-6-(1-(1-((4-(2-(2,6-dioxopiperidin- |
| | 3-yl)-1,3-dioxoisoindolin-4-yl)morpholin-2-yl)methyl)piperidin-4-yl)-1H-pyrazol-4- |
| | yl)pyrazine-2-carboxylate |
| TDH-311 | 2-((4-fluorophenyl)amino)-2-oxo-1-phenylethyl 3-amino-6-(1-(1-((4-(2-(2,6-dioxopiperidin- |
| | 3-yl)-1,3-dioxoisoindolin-5-yl)morpholin-2-yl)methyl)piperidin-4-yl)-1H-pyrazol-4- |
| | yl)pyrazine-2-carboxylate |
| TDH-312 | 2-((4-fluorophenyl)amino)-2-oxo-1-phenylethyl 3-amino-6-(1-(1-(2-(1-(2-(((S)-1-((2R,4S)-4- |
| | hydroxy-2-((4-(4-methylthiazol-5-yl)benzyl)carbamoyl)pyrrolidin-1-yl)-3,3-dimethyl-1- |
| | oxobutan-2-yl)amino)-2-oxoethyl)piperidin-4-yl)ethyl)piperidin-4-yl)-1H-pyrazol-4- |
| | yl)pyrazine-2-carboxylate |
| TDH-328 | (R)-2-((4-fluorophenyl)amino)-2-oxo-1-phenylethyl 3-amino-6-(1-(1-((1-(3-(2-(2,6- |
| | dioxopiperidin-3-yl)-1,3-dioxoisoindolin-5-yl)propanoyl)piperidin-4-yl)methyl)piperidin-4-yl)- |
| | 1H-pyrazol-4-yl)pyrazine-2-carboxylate |
| TDH-330 | (R)-2-((4-fluorophenyl)amino)-2-oxo-1-phenylethyl 3-amino-6-(1-(1-(2-(1-(2-((3-(2,6- |
| | dioxopiperidin-3-yl)-4-oxo-3,4-dihydrobenzo[d][1,2,3]triazin-6-yl)oxy)acetyl)piperidin-4- |
| | yl)ethyl)piperidin-4-yl)-1H-pyrazol-4-yl)pyrazine-2-carboxylate |
| TDH-331 | (R)-2-((4-fluorophenyl)amino)-2-oxo-1-phenylethyl 3-amino-6-(1-(1-(2-((1-(2-((2-(2,6- |
| | dioxopiperidin-3-yl)-1,3-dioxoisoindolin-4-yl)oxy)acetyl)piperidin-4-yl)oxy)ethyl)piperidin-4- |
| | yl)-1H-pyrazol-4-yl)pyrazine-2-carboxylate |
| TDH-332 | (R)-2-((4-fluorophenyl)amino)-2-oxo-1-phenylethyl 3-amino-6-(1-(1-(2-((1-(2-((2-(2,6- |
| | dioxopiperidin-3-yl)-1,3-dioxoisoindolin-5-yl)oxy)acetyl)piperidin-4-yl)oxy)ethyl)piperidin-4- |
| | yl)-1H-pyrazol-4-yl)pyrazine-2-carboxylate |
| TDH-333 | (R)-2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)ethyl)piperidin-4-yl)-1H-pyrazol-4-yl)pyrazine-2-carboxylate |
| TDH-334 | (R)-2-((4-fluorophenyl)amino)-2-oxo-1-phenylethyl 3-amino-6-(1-(1-(2-(1-((3-(2,6- |
| | dioxopiperidin-3-yl)-4-oxo-3,4-dihydrobenzo[d][1,2,3]triazin-6-yl)glycyl)piperidin-4- |
| | yl)ethyl)piperidin-4-yl)-1H-pyrazol-4-yl)pyrazine-2-carboxylate |
| TDH-336 | (R)-2-((4-fluorophenyl)amino)-2-oxo-1-phenylethyl 3-amino-6-(1-(1-((1-(3-(2-(2,6- |
| | dioxopiperidin-3-yl)-1,3-dioxoisoindolin-4-yl)propanoyl)piperidin-4-yl)methyl)piperidin-4-yl)- |
| | 1H-pyrazol-4-yl)pyrazine-2-carboxylate |
| TDH-337 | (R)-2-((4-fluorophenyl)amino)-2-oxo-1-phenylethyl 3-amino-6-(1-(1-(2-(1-(2-((2-(2,6- |
| | dioxopiperidin-3-yl)-1,3-dioxoisoindolin-4-yl)oxy)acetyl)piperidin-4-yl)acetyl)piperidin-4-yl)- |
| | 1H-pyrazol-4-yl)pyrazine-2-carboxylate |
| TDH-338 | (R)-2-((4-fluorophenyl)amino)-2-oxo-1-phenylethyl 3-amino-6-(1-(1-(2-(1-(2-((2-(2,6- |
| | dioxopiperidin-3-yl)-1,3-dioxoisoindolin-5-yl)oxy)acetyl)piperidin-4-yl)acetyl)piperidin-4-yl)- |
| | 1H-pyrazol-4-yl)pyrazine-2-carboxylate |
| TDH-339 | (R)-2-((4-fluorophenyl)amino)-2-oxo-1-phenylethyl 3-amino-6-(1-(1-(2-(1-((2-(2,6- |
| | dioxopiperidin-3-yl)-1,3-dioxoisoindolin-4-yl)glycyl)piperidin-4-yl)acetyl)piperidin-4-yl)-1H- |
| | pyrazol-4-yl)pyrazine-2-carboxylate |
| TDH-340 | (R)-2-((4-fluorophenyl)amino)-2-oxo-1-phenylethyl 3-amino-6-(1-(1-(2-(1-((2-(2,6- |
| | dioxopiperidin-3-yl)-1,3-dioxoisoindolin-5-yl)glycyl)piperidin-4-yl)acetyl)piperidin-4-yl)-1H- |
| | pyrazol-4-yl)pyrazine-2-carboxylate |
| TDH-341 | (R)-2-((4-fluorophenyl)amino)-2-oxo-1-phenylethyl 3-amino-6-(1-(1-(2-(1-(1-(2-(2,6- |
| | dioxopiperidin-3-yl)-1,3-dioxoisoindolin-4-yl)piperidine-4-carbonyl)piperidin-4- |
| | yl)acetyl)piperidin-4-yl)-1H-pyrazol-4-yl)pyrazine-2-carboxylate |
| TDH-342 | (R)-2-((4-fluorophenyl)amino)-2-oxo-1-phenylethyl 3-amino-6-(1-(1-(2-(1-(1-(2-(2,6- |
| | dioxopiperidin-3-yl)-1,3-dioxoisoindolin-5-yl)piperidine-4-carbonyl)piperidin-4- |
| | yl)acetyl)piperidin-4-yl)-1H-pyrazol-4-yl)pyrazine-2-carboxylate |
| TDH-343 | (R)-2-((4-fluorophenyl)amino)-2-oxo-1-phenylethyl 3-amino-6-(1-(1-(2-(1-(2-(2,6- |
| | dioxopiperidin-3-yl)-1,3-dioxoisoindolin-4-yl)piperidin-4-yl)acetyl)piperidin-4-yl)-1H-pyrazol- |
| | 4-yl)pyrazine-2-carboxylate |
| TDH-344 | (R)-2-((4-fluorophenyl)amino)-2-oxo-1-phenylethyl 3-amino-6-(1-(1-(2-(1-(2-(2,6- |
| | dioxopiperidin-3-yl)-1,3-dioxoisoindolin-5-yl)piperidin-4-yl)acetyl)piperidin-4-yl)-1H-pyrazol- |
| | 4-yl)pyrazine-2-carboxylate |
| TDH-345 | (R)-2-((4-fluorophenyl)amino)-2-oxo-1-phenylethyl 3-amino-6-(1-(1-(((1r,4r)-4-((2-((2-(2,6- |
| | dioxopiperidin-3-yl)-1,3-dioxoisoindolin-4- |
| | yl)oxy)acetamido)methyl)cyclohexyl)methyl)piperidin-4-yl)-1H-pyrazol-4-yl)pyrazine-2- |
| | carboxylate |
| TDH-346 | (R)-2-((4-fluorophenyl)amino)-2-oxo-1-phenylethyl 3-amino-6-(1-(1-(((1r,4r)-4-((2-((2-(2,6- |
| | dioxopiperidin-3-yl)-1,3-dioxoisoindolin-5- |
| | yl)oxy)acetamido)methyl)cyclohexyl)methyl)piperidin-4-yl)-1H-pyrazol-4-yl)pyrazine-2- |
| | carboxylate |
| TDH-347 | (R)-2-((4-fluorophenyl)amino)-2-oxo-1-phenylethyl 3-amino-6-(1-(1-(((1r,4r)-4-((2-((2-(2,6- |
| | dioxopiperidin-3-yl)-1,3-dioxoisoindolin-4- |
| | yl)amino)acetamido)methyl)cyclohexyl)methyl)piperidin-4-yl)-1H-pyrazol-4-yl)pyrazine-2- |
| | carboxylate |
| TDH-348 | (R)-2-((4-fluorophenyl)amino)-2-oxo-1-phenylethyl 3-amino-6-(1-(1-(((1r,4r)-4-((2-((2-(2,6- |
| | dioxopiperidin-3-yl)-1,3-dioxoisoindolin-5- |
| | yl)amino)acetamido)methyl)cyclohexyl)methyl)piperidin-4-yl)-1H-pyrazol-4-yl)pyrazine-2- |
| | carboxylate |
| TDH-349 | (R)-2-((4-fluorophenyl)amino)-2-oxo-1-phenylethyl 3-amino-6-(1-(1-(((1r,4r)-4-((1-(2-(2,6- |
| | dioxopiperidin-3-yl)-1,3-dioxoisoindolin-4-yl)piperidine-4- |
| | carboxamido)methyl)cyclohexyl)methyl)piperidin-4-yl)-1H-pyrazol-4-yl)pyrazine-2- |
| | carboxylate |
| TDH-350 | (R)-2-((4-fluorophenyl)amino)-2-oxo-1-phenylethyl 3-amino-6-(1-(1-(((1r,4r)-4-((1-(2-(2,6- |
| | dioxopiperidin-3-yl)-1,3-dioxoisoindolin-5-yl)piperidine-4- |
| | carboxamido)methyl)cyclohexyl)methyl)piperidin-4-yl)-1H-pyrazol-4-yl)pyrazine-2- |
| | carboxylate |
| TDH-351 | (R)-2-((4-fluorophenyl)amino)-2-oxo-1-phenylethyl 3-amino-6-(1-(1-(((1r,4r)-4-(((2-(2,6- |
| | dioxopiperidin-3-yl)-1,3-dioxoisoindolin-4-yl)amino)methyl)cyclohexyl)methyl)piperidin-4- |
| | yl)-1H-pyrazol-4-yl)pyrazine-2-carboxylate |
| TDH-352 | (R)-2-((4-fluorophenyl)amino)-2-oxo-1-phenylethyl 3-amino-6-(1-(1-(((1r,4r)-4-(((2-(2,6- |
| | dioxopiperidin-3-yl)-1,3-dioxoisoindolin-5-yl)amino) methyl)cyclohexyl)methyl)piperidin-4- |
| | yl)-1H-pyrazol-4-yl)pyrazine-2-carboxylate |
| TDH-353 | (R)-2-((4-fluorophenyl)amino)-2-oxo-1-phenylethyl 3-amino-6-(1-(1-(1-((2-(2,6- |
| | dioxopiperidin-3-yl)-1,3-dioxoisoindolin-5-yl)glycyl)piperidine-4-carbonyl)piperidin-4-yl)-1H- |
| | pyrazol-4-yl)pyrazine-2-carboxylate |
| TDH-354 | (R)-2-((4-fluorophenyl)amino)-2-oxo-1-phenylethyl 3-amino-6-(1-(1-(1-(1-(2-((2-(2,6- |
| | dioxopiperidin-3-yl)-1,3-dioxoisoindolin-4-yl)oxy)acetyl)piperidin-4-yl)-1H-imidazole-4- |
| | carbonyl)piperidin-4-yl)-1H-pyrazol-4-yl)pyrazine-2-carboxylate |
| TDH-355 | (R)-2-((4-fluorophenyl)amino)-2-oxo-1-phenylethyl 3-amino-6-(1-(1-(1-(1-(2-((2-(2,6- |
| | dioxopiperidin-3-yl)-1,3-dioxoisoindolin-5-yl)oxy)acetyl)piperidin-4-yl)-1H-imidazole-4- |
| | carbonyl)piperidin-4-yl)-1H-pyrazol-4-yl)pyrazine-2-carboxylate |
| TDH-356 | (R)-2-((4-fluorophenyl)amino)-2-oxo-1-phenylethyl 3-amino-6-(1-(1-(1-(1-((2-(2,6- |
| | dioxopiperidin-3-yl)-1,3-dioxoisoindolin-4-yl)glycyl)piperidin-4-yl)-1H-imidazole-4- |
| | carbonyl)piperidin-4-yl)-1H-pyrazol-4-yl)pyrazine-2-carboxylate |
| TDH-357 | (R)-2-((4-fluorophenyl)amino)-2-oxo-1-phenylethyl 3-amino-6-(1-(1-(3-(4-((2-(2,6- |
| | dioxopiperidin-3-yl)-1,3-dioxoisoindolin-5-yl)glycyl)piperazin-1-yl)propyl)piperidin-4-yl)-1H- |
| | pyrazol-4-yl)pyrazine-2-carboxylate |
| TDH-358 | (R)-2-((4-fluorophenyl)amino)-2-oxo-1-phenylethyl 3-amino-6-(1-(1-(3-(4-(2-((2-(2,6- |
| | dioxopiperidin-3-yl)-1,3-dioxoisoindolin-5-yl)oxy)acetyl)piperazin-1-yl)propyl)piperidin-4- |
| | yl)-1H-pyrazol-4-yl)pyrazine-2-carboxylate |
| TDH-359 | (R)-2-((4-fluorophenyl)amino)-2-oxo-1-phenylethyl 3-amino-6-(1-((3R)-1-((1-(2-((2-(2,6- |
| | dioxopiperidin-3-yl)-1,3-dioxoisoindolin-5-yl)oxy)acetyl)piperidin-4-yl)methyl)pyrrolidin-3- |
| | yl)-1H-pyrazol-4-yl)pyrazine-2-carboxylate |
| TDH-360 | (R)-2-((4-fluorophenyl)amino)-2-oxo-1-phenylethyl 3-amino-6-(1-(1-((1-((2-(2,6- |
| | dioxopiperidin-3-yl)-1,3-dioxoisoindolin-5-yl)glycyl)piperidin-4-yl)methyl)azetidin-3-yl)-1H- |
| | pyrazol-4-yl)pyrazine-2-carboxylate |
| TDH-361 | (R)-2-((4-fluorophenyl)amino)-2-oxo-1-phenylethyl 3-amino-6-(1-(1-(1-(1-((2-(2,6- |
| | dioxopiperidin-3-yl)-1,3-dioxoisoindolin-5-yl)glycyl)piperidin-4-yl)-1H-imidazole-4- |
| | carbonyl)piperidin-4-yl)-1H-pyrazol-4-yl)pyrazine-2-carboxylate |
| TDH-362 | (R)-2-((4-fluorophenyl)amino)-2-oxo-1-phenylethyl 3-amino-6-(1-(1-(1-(1-(1-(2-(2,6- |
| | dioxopiperidin-3-yl)-1,3-dioxoisoindolin-4-yl)piperidine-4-carbonyl)piperidin-4-yl)-1H- |
| | imidazole-4-carbonyl)piperidin-4-yl)-1H-pyrazol-4-yl)pyrazine-2-carboxylate |
| TDH-363 | (R)-2-((4-fluorophenyl)amino)-2-oxo-1-phenylethyl 3-amino-6-(1-(1-(1-(1-(1-(2-(2,6- |
| | dioxopiperidin-3-yl)-1,3-dioxoisoindolin-5-yl)piperidine-4-carbonyl)piperidin-4-yl)-1H- |
| | imidazole-4-carbonyl)piperidin-4-yl)-1H-pyrazol-4-yl)pyrazine-2-carboxylate |
| TDH-364 | (R)-2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-4-yl)piperidin-4-yl)-1H-imidazole-4- |
| | carbonyl)piperidin-4-yl)-1H-pyrazol-4-yl)pyrazine-2-carboxylate |
| TDH-365 | (R)-2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-5-yl)piperidin-4-yl)-1H-imidazole-4- |
| | carbonyl)piperidin-4-yl)-1H-pyrazol-4-yl)pyrazine-2-carboxylate |
| TDH-366 | (R)-2-((4-fluorophenyl)amino)-2-oxo-1-phenylethyl 3-amino-6-(1-(1-(2-(1-(2-(1-(2-(2,6- |
| | dioxopiperidin-3-yl)-1,3-dioxoisoindolin-4-yl)piperidin-4-yl)acetyl)piperidin-4- |
| | yl)ethyl)piperidin-4-yl)-1H-pyrazol-4-yl)pyrazine-2-carboxylate |
| TDH-367 | (R)-2-((4-fluorophenyl)amino)-2-oxo-1-phenylethyl 3-amino-6-(1-(1-(2-(1-(2-(1-(2-(2,6- |
| | dioxopiperidin-3-yl)-1,3-dioxoisoindolin-5-yl)piperidin-4-yl)acetyl)piperidin-4- |
| | yl)ethyl)piperidin-4-yl)-1H-pyrazol-4-yl)pyrazine-2-carboxylate |
| TDH-368 | (R)-2-((4-fluorophenyl)amino)-2-oxo-1-phenylethyl 3-amino-6-(1-(1-(3-(4-(2-(1-(2-(2,6- |
| | dioxopiperidin-3-yl)-1,3-dioxoisoindolin-4-yl)piperidin-4-yl)acetyl)piperazin-1- |
| | yl)propyl)piperidin-4-yl)-1H-pyrazol-4-yl)pyrazine-2-carboxylate |
| TDH-369 | (R)-2-((4-fluorophenyl)amino)-2-oxo-1-phenylethyl 3-amino-6-(1-(1-(3-(4-(2-(1-(2-(2,6- |
| | dioxopiperidin-3-yl)-1,3-dioxoisoindolin-5-yl)piperidin-4-yl)acetyl)piperazin-1- |
| | yl)propyl)piperidin-4-yl)-1H-pyrazol-4-yl)pyrazine-2-carboxylate |
| TDH-370 | (R)-2-((4-fluorophenyl)amino)-2-oxo-1-phenylethyl 3-amino-6-(1-(1-(3-(4-(2-((3-(2,6- |
| | dioxopiperidin-3-yl)-4-oxo-3,4-dihydrobenzo[d][1,2,3]triazin-6-yl)oxy)acetyl)piperazin-1- |
| | yl)propyl)piperidin-4-yl)-1H-pyrazol-4-yl)pyrazine-2-carboxylate |
| TDH-371 | (R)-2-((4-fluorophenyl)amino)-2-oxo-1-phenylethyl 3-amino-6-(1-(1-(2-((1-(2-(1-(2-(2,6- |
| | dioxopiperidin-3-yl)-1,3-dioxoisoindolin-4-yl)piperidin-4-yl)acetyl)piperidin-4- |
| | yl)oxy)ethyl)piperidin-4-yl)-1H-pyrazol-4-yl)pyrazine-2-carboxylate |
| TDH-372 | (R)-2-((4-fluorophenyl)amino)-2-oxo-1-phenylethyl 3-amino-6-(1-(1-(2-((1-(2-(1-(2-(2,6- |
| | dioxopiperidin-3-yl)-1,3-dioxoisoindolin-5-yl)piperidin-4-yl)acetyl)piperidin-4- |
| | yl)oxy)ethyl)piperidin-4-yl)-1H-pyrazol-4-yl)pyrazine-2-carboxylate |
| TDH-373 | (R)-2-((4-fluorophenyl)amino)-2-oxo-1-phenylethyl 3-amino-6-(1-(1-(2-((1-(2-((3-(2,6- |
| | dioxopiperidin-3-yl)-4-oxo-3,4-dihydrobenzo[d][1,2,3]triazin-6-yl)oxy)acetyl)piperidin-4- |
| | yl)oxy)ethyl)piperidin-4-yl)-1H-pyrazol-4-yl)pyrazine-2-carboxylate |
| TDH-374 | (R)-2-((4-fluorophenyl)amino)-2-oxo-1-phenylethyl 3-amino-6-(1-(1-(1-(2-(1-(2-(2,6- |
| | dioxopiperidin-3-yl)-1,3-dioxoisoindolin-4-yl)piperidin-4-yl)acetyl)piperidine-4- |
| | carbonyl)piperidin-4-yl)-1H-pyrazol-4-yl)pyrazine-2-carboxylate |
| TDH-375 | (R)-2-((4-fluorophenyl)amino)-2-oxo-1-phenylethyl 3-amino-6-(1-(1-(1-(2-(1-(2-(2,6- |
| | dioxopiperidin-3-yl)-1,3-dioxoisoindolin-5-yl)piperidin-4-yl)acetyl)piperidine-4- |
| | carbonyl)piperidin-4-yl)-1H-pyrazol-4-yl)pyrazine-2-carboxylate |
| TDH-376 | (R)-2-((4-fluorophenyl)amino)-2-oxo-1-phenylethyl 3-amino-6-(1-(1-(2-(1-(3-(2-(2,6- |
| | dioxopiperidin-3-yl)-1,3-dioxoisoindolin-4-yl)propanoyl)piperidin-4-yl)ethyl)piperidin-4-yl)- |
| | 1H-pyrazol-4-yl)pyrazine-2-carboxylate |
| TDH-377 | (R)-2-((4-fluorophenyl)amino)-2-oxo-1-phenylethyl 3-amino-6-(1-(1-(2-(1-(3-(2-(2,6- |
| | dioxopiperidin-3-yl)-1,3-dioxoisoindolin-5-yl)propanoyl)piperidin-4-yl)ethyl)piperidin-4-yl)- |
| | 1H-pyrazol-4-yl)pyrazine-2-carboxylate |
| TDH-378 | (R)-2-((4-fluorophenyl)amino)-2-oxo-1-phenylethyl 3-amino-6-(1-(1-(2-((1-(3-(2-(2,6- |
| | dioxopiperidin-3-yl)-1,3-dioxoisoindolin-4-yl)propanoyl)piperidin-4-yl)oxy)ethyl)piperidin-4- |
| | yl)-1H-pyrazol-4-yl)pyrazine-2-carboxylate |
| TDH-379 | (R)-2-((4-fluorophenyl)amino)-2-oxo-1-phenylethyl 3-amino-6-(1-(1-(2-((1-(3-(2-(2,6- |
| | dioxopiperidin-3-yl)-1,3-dioxoisoindolin-5-yl)propanoyl)piperidin-4-yl)oxy)ethyl)piperidin-4- |
| | yl)-1H-pyrazol-4-yl)pyrazine-2-carboxylate |
| TDH-380 | (R)-2-((4-fluorophenyl)amino)-2-oxo-1-phenylethyl 3-amino-6-(1-(1-(1-(3-(2-(2,6- |
| | dioxopiperidin-3-yl)-1,3-dioxoisoindolin-4-yl)propanoyl)piperidine-4-carbonyl)piperidin-4- |
| | yl)-1H-pyrazol-4-yl)pyrazine-2-carboxylate |
| TDH-381 | (R)-2-((4-fluorophenyl)amino)-2-oxo-1-phenylethyl 3-amino-6-(1-(1-(1-(3-(2-(2,6- |
| | dioxopiperidin-3-yl)-1,3-dioxoisoindolin-5-yl)propanoyl)piperidine-4-carbonyl)piperidin-4- |
| | yl)-1H-pyrazol-4-yl)pyrazine-2-carboxylate |
| TDH-382 | (R)-2-((4-fluorophenyl)amino)-2-oxo-1-phenylethyl 3-amino-6-(1-(1-(3-(4-(3-(2-(2,6- |
| | dioxopiperidin-3-yl)-1,3-dioxoisoindolin-4-yl)propanoyl)piperazin-1-yl)propyl)piperidin-4-yl)- |
| | 1H-pyrazol-4-yl)pyrazine-2-carboxylate |
| TDH-383 | (R)-2-((4-fluorophenyl)amino)-2-oxo-1-phenylethyl 3-amino-6-(1-(1-(3-(4-(3-(2-(2,6- |
| | dioxopiperidin-3-yl)-1,3-dioxoisoindolin-5-yl)propanoyl)piperazin-1-yl)propyl)piperidin-4-yl)- |
| | 1H-pyrazol-4-yl)pyrazine-2-carboxylate |
| TDH-384 | (R)-2-((4-fluorophenyl)amino)-2-oxo-1-phenylethyl 3-amino-6-(1-(1-((1-(3-(2-(2,6- |
| | dioxopiperidin-3-yl)-1-oxoisoindolin-4-yl)propanoyl)piperidin-4-yl)methyl)piperidin-4-yl)-1H- |
| | pyrazol-4-yl)pyrazine-2-carboxylate |
| TDH-385 | (R)-2-((4-fluorophenyl)amino)-2-oxo-1-phenylethyl 3-amino-6-(1-(1-(2-(1-(3-(2-(2,6- |
| | dioxopiperidin-3-yl)-1-oxoisoindolin-4-yl)propanoyl)piperidin-4-yl)ethyl)piperidin-4-yl)-1H- |
| | pyrazol-4-yl)pyrazine-2-carboxylate |
| TDH-386 | (R)-2-((4-fluorophenyl)amino)-2-oxo-1-phenylethyl 3-amino-6-(1-(1-(3-(4-(3-(2-(2,6- |
| | dioxopiperidin-3-yl)-1-oxoisoindolin-4-yl)propanoyl)piperazin-1-yl)propyl)piperidin-4-yl)-1H- |
| | pyrazol-4-yl)pyrazine-2-carboxylate |
| TDH-387 | (R)-2-((4-fluorophenyl)amino)-2-oxo-1-phenylethyl 3-amino-6-(1-(1-(2-((1-(3-(2-(2,6- |
| | dioxopiperidin-3-yl)-1-oxoisoindolin-4-yl)propanoyl)piperidin-4-yl)oxy)ethyl)piperidin-4-yl)- |
| | 1H-pyrazol-4-yl)pyrazine-2-carboxylate |
| TDH-388 | (R)-2-((4-fluorophenyl)amino)-2-oxo-1-phenylethyl 3-amino-6-(1-(1-(1-(3-(2-(2,6- |
| | dioxopiperidin-3-yl)-1-oxoisoindolin-4-yl)propanoyl)piperidine-4-carbonyl)piperidin-4-yl)- |
| | 1H-pyrazol-4-yl)pyrazine-2-carboxylate |
| TDH-389 | (R)-2-((4-fluorophenyl)amino)-2-oxo-1-phenylethyl 3-amino-6-(1-(1-(3-(4-(2-((2-(2,6- |
| | dioxopiperidin-3-yl)-1,3-dioxoisoindolin-5-yl)oxy)acetyl)piperazin-1-yl)propyl)piperidin-4- |
| | yl)-1H-pyrazol-4-yl)pyrazine-2-carboxylate hydrochloride |
| TDH-390 | (R)-2-((4-fluorophenyl)amino)-2-oxo-1-phenylethyl 3-amino-6-(1-(1-((4-(2-((2-(2,6- |
| | dioxopiperidin-3-yl)-1,3-dioxoisoindolin-5-yl)oxy)acetyl)morpholin-2-yl)methyl)piperidin-4- |
| | yl)-1H-pyrazol-4-yl)pyrazine-2-carboxylate |
| TDH-391 | (R)-2-((4-fluorophenyl)amino)-2-oxo-1-phenylethyl 3-amino-6-(1-(1-(2-(1-((2-(2,6- |
| | dioxopiperidin-3-yl)-1,3-dioxoisoindolin-4-yl)glycyl)piperidin-4-yl)ethyl)piperidin-4-yl)-1H- |
| | pyrazol-4-yl)pyrazine-2-carboxylate hydrochloride |
| TDH-392 | (R)-2-((4-fluorophenyl)amino)-2-oxo-1-phenylethyl 3-amino-6-(1-(1-(2-(1-(2-((2-(2,6- |
| | dioxopiperidin-3-yl)-1,3-dioxoisoindolin-5-yl)oxy)acetyl)piperidin-4-yl)ethyl)piperidin-4-yl)- |
| | 1H-pyrazol-4-yl)pyrazine-2-carboxylate hydrochloride |
| TDH-393 | (R)-2-((4-fluorophenyl)amino)-2-oxo-1-phenylethyl 3-amino-6-(1-(1-(2-((1-(2-((2-(2,6- |
| | dioxopiperidin-3-yl)-1,3-dioxoisoindolin-4-yl)oxy)acetyl)piperidin-4-yl)oxy)acetyl)piperidin- |
| | 4-yl)-1H-pyrazol-4-yl)pyrazine-2-carboxylate |
| TDH-394 | (R)-2-((4-fluorophenyl)amino)-2-oxo-1-phenylethyl 3-amino-6-(1-(1-(2-((1-(2-((2-(2,6- |
| | dioxopiperidin-3-yl)-1,3-dioxoisoindolin-5-yl)oxy)acetyl)piperidin-4-yl)oxy)acetyl)piperidin- |
| | 4-yl)-1H-pyrazol-4-yl)pyrazine-2-carboxylate |
| TDH-395 | (R)-2-((4-fluorophenyl)amino)-2-oxo-1-phenylethyl 3-amino-6-(1-(1-(2-((1-((2-(2,6- |
| | dioxopiperidin-3-yl)-1,3-dioxoisoindolin-4-yl)glycyl)piperidin-4-yl)oxy)acetyl)piperidin-4-yl)- |
| | 1H-pyrazol-4-yl)pyrazine-2-carboxylate |
| TDH-396 | (R)-2-((4-fluorophenyl)amino)-2-oxo-1-phenylethyl 3-amino-6-(1-(1-(2-((1-((2-(2,6- |
| | dioxopiperidin-3-yl)-1,3-dioxoisoindolin-5-yl)glycyl)piperidin-4-yl)oxy)acetyl)piperidin-4-yl)- |
| | 1H-pyrazol-4-yl)pyrazine-2-carboxylate |
| TDH-397 | (R)-2-((4-fluorophenyl)amino)-2-oxo-1-phenylethyl 3-amino-6-(1-(1-(2-((1-(1-(2-(2,6- |
| | dioxopiperidin-3-yl)-1,3-dioxoisoindolin-4-yl)piperidine-4-carbonyl)piperidin-4- |
| | yl)oxy)acetyl)piperidin-4-yl)-1H-pyrazol-4-yl)pyrazine-2-carboxylate |
| TDH-398 | (R)-2-((4-fluorophenyl)amino)-2-oxo-1-phenylethyl 3-amino-6-(1-(1-(2-((1-(1-(2-(2,6- |
| | dioxopiperidin-3-yl)-1,3-dioxoisoindolin-5-yl)piperidine-4-carbonyl)piperidin-4- |
| | yl)oxy)acetyl)piperidin-4-yl)-1H-pyrazol-4-yl)pyrazine-2-carboxylate |
| TDH-399 | (R)-2-((4-fluorophenyl)amino)-2-oxo-1-phenylethyl 3-amino-6-(1-(1-(2-((1-(2-(2,6- |
| | dioxopiperidin-3-yl)-1,3-dioxoisoindolin-4-yl)piperidin-4-yl)oxy)acetyl)piperidin-4-yl)-1H- |
| | pyrazol-4-yl)pyrazine-2-carboxylate |
| TDH-400 | (R)-2-((4-fluorophenyl)amino)-2-oxo-1-phenylethyl 3-amino-6-(1-(1-(2-((1-(2-(2,6- |
| | dioxopiperidin-3-yl)-1,3-dioxoisoindolin-5-yl)piperidin-4-yl)oxy)acetyl)piperidin-4-yl)-1H- |
| | pyrazol-4-yl)pyrazine-2-carboxylate |
| TDH-401 | (R)-2-((4-fluorophenyl)amino)-2-oxo-1-phenylethyl 3-amino-6-(1-(1-(2-(1-(1-(2-(2,6- |
| | dioxopiperidin-3-yl)-1,3-dioxoisoindolin-5-yl)azetidine-3-carbonyl)piperidin-4- |
| | yl)acetyl)piperidin-4-yl)-1H-pyrazol-4-yl)pyrazine-2-carboxylate |
| TDH-402 | (R)-2-((4-fluorophenyl)amino)-2-oxo-1-phenylethyl 3-amino-6-(1-(1-(2-(1-(2-(1-(2-(2,6- |
| | dioxopiperidin-3-yl)-1,3-dioxoisoindolin-5-yl)piperidin-4-yl)acetyl)piperidin-4- |
| | yl)acetyl)piperidin-4-yl)-1H-pyrazol-4-yl)pyrazine-2-carboxylate |
| TDH-403 | (R)-2-((4-fluorophenyl)amino)-2-oxo-1-phenylethyl 3-amino-6-(1-(1-(2-(1-(3-(2-(2,6- |
| | dioxopiperidin-3-yl)-1,3-dioxoisoindolin-5-yl)propanoyl)piperidin-4-yl)acetyl)piperidin-4-yl)- |
| | 1H-pyrazol-4-yl)pyrazine-2-carboxylate |
| TDH-404 | (R)-2-((4-fluorophenyl)amino)-2-oxo-1-phenylethyl 3-amino-6-(1-(1-(2-(1-(3-(2-(2,6- |
| | dioxopiperidin-3-yl)-1-oxoisoindolin-4-yl)propanoyl)piperidin-4-yl)acetyl)piperidin-4-yl)-1H- |
| | pyrazol-4-yl)pyrazine-2-carboxylate |
| TDH-405 | (R)-2-((4-fluorophenyl)amino)-2-oxo-1-phenylethyl 3-amino-6-(1-(1-(2-(1-((3-(2,6- |
| | dioxopiperidin-3-yl)-4-oxo-3,4-dihydrobenzo[d][1,2,3]triazin-6-yl)glycyl)piperidin-4- |
| | yl)acetyl)piperidin-4-yl)-1H-pyrazol-4-yl)pyrazine-2-carboxylate |
| TDH-406 | (R)-2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)acetyl)piperidin-4-yl)-1H-pyrazol-4-yl)pyrazine-2-carboxylate |
| TDH-407 | (R)-2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)acetyl)piperidin-4-yl)-1H-pyrazol-4-yl)pyrazine-2-carboxylate |
| TDH-408 | (R)-2-((4-fluorophenyl)amino)-2-oxo-1-phenylethyl 3-amino-6-(1-(1-(1-((3-(2,6- |
| | dioxopiperidin-3-yl)-4-oxo-3,4-dihydrobenzo[d][1,2,3]triazin-6-yl)glycyl)piperidine-4- |
| | carbonyl)piperidin-4-yl)-1H-pyrazol-4-yl)pyrazine-2-carboxylate |
| TDH-409 | (R)-2-((4-fluorophenyl)amino)-2-oxo-1-phenylethyl 3-amino-6-(1-(1-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidine-4-carbonyl)piperidin-4-yl)-1H-pyrazol-4-yl)pyrazine-2-carboxylate |
| TDH-410 | (R)-2-((4-fluorophenyl)amino)-2-oxo-1-phenylethyl 3-amino-6-(1-(1-(1-(1-(3-(2,6- |
| | dioxopiperidin-3-yl)-4-oxo-3,4-dihydrobenzo[d][1,2,3]triazin-6-yl)piperidine-4- |
| | carbonyl)piperidine-4-carbonyl)piperidin-4-yl)-1H-pyrazol-4-yl)pyrazine-2-carboxylate |
| TDH-411 | (R)-2-((4-fluorophenyl)amino)-2-oxo-1-phenylethyl 3-amino-6-(1-(1-(2-(1-((3-(2,6- |
| | dioxopiperidin-3-yl)-4-oxo-3,4-dihydrobenzo[d][1,2,3]triazin-5-yl)glycyl)piperidin-4- |
| | yl)acetyl)piperidin-4-yl)-1H-pyrazol-4-yl)pyrazine-2-carboxylate |
| TDH-412 | (R)-2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-5-yl)piperidine-4- |
| | carbonyl)piperidin-4-yl)acetyl)piperidin-4-yl)-1H-pyrazol-4-yl)pyrazine-2-carboxylate |
| TDH-413 | (R)-2-((4-fluorophenyl)amino)-2-oxo-1-phenylethyl 3-amino-6-(1-(1-(1-((3-(2,6- |
| | dioxopiperidin-3-yl)-4-oxo-3,4-dihydrobenzo[d][1,2,3]triazin-5-yl)glycyl)piperidine-4- |
| | carbonyl)piperidin-4-yl)-1H-pyrazol-4-yl)pyrazine-2-carboxylate |
| TDH-414 | (R)-2-((4-fluorophenyl)amino)-2-oxo-1-phenylethyl 3-amino-6-(1-(1-(1-(1-(3-(2,6- |
| | dioxopiperidin-3-yl)-4-oxo-3,4-dihydrobenzo[d][1,2,3]triazin-5-yl)piperidine-4- |
| | carbonyl)piperidine-4-carbonyl)piperidin-4-yl)-1H-pyrazol-4-yl)pyrazine-2-carboxylate |
| TDH-415 | (R)-2-((4-fluorophenyl)amino)-2-oxo-1-phenylethyl 3-amino-6-(1-(1-(3-(1-(2-((2-(2,6- |
| | dioxopiperidin-3-yl)-1,3-dioxoisoindolin-4-yl)oxy)acetyl)piperidin-4-yl)propanoyl)piperidin- |
| | 4-yl)-1H-pyrazol-4-yl)pyrazine-2-carboxylate |
| TDH-416 | (R)-2-((4-fluorophenyl)amino)-2-oxo-1-phenylethyl 3-amino-6-(1-(1-(3-(1-(2-((2-(2,6- |
| | dioxopiperidin-3-yl)-1,3-dioxoisoindolin-5-yl)oxy)acetyl)piperidin-4-yl)propanoyl)piperidin- |
| | 4-yl)-1H-pyrazol-4-yl)pyrazine-2-carboxylate |
| TDH-417 | (R)-2-((4-fluorophenyl)amino)-2-oxo-1-phenylethyl 3-amino-6-(1-(1-(3-(1-(1-(2-(2,6- |
| | dioxopiperidin-3-yl)-1,3-dioxoisoindolin-4-yl)piperidine-4-carbonyl)piperidin-4- |
| | yl)propanoyl)piperidin-4-yl)-1H-pyrazol-4-yl)pyrazine-2-carboxylate |
| TDH-418 | (R)-2-((4-fluorophenyl)amino)-2-oxo-1-phenylethyl 3-amino-6-(1-(1-(3-(1-(1-(2-(2,6- |
| | dioxopiperidin-3-yl)-1,3-dioxoisoindolin-5-yl)piperidine-4-carbonyl)piperidin-4- |
| | yl)propanoyl)piperidin-4-yl)-1H-pyrazol-4-yl)pyrazine-2-carboxylate |
| TDH-419 | (R)-2-((4-fluorophenyl)amino)-2-oxo-1-phenylethyl 3-amino-6-(1-(1-(3-(1-(2-(2,6- |
| | dioxopiperidin-3-yl)-1,3-dioxoisoindolin-4-yl)piperidin-4-yl)propanoyl)piperidin-4-yl)-1H- |
| | pyrazol-4-yl)pyrazine-2-carboxylate |
| TDH-420 | (R)-2-((4-fluorophenyl)amino)-2-oxo-1-phenylethyl 3-amino-6-(1-(1-(3-(1-(2-(2,6- |
| | dioxopiperidin-3-yl)-1,3-dioxoisoindolin-5-yl)piperidin-4-yl)propanoyl)piperidin-4-yl)-1H- |
| | pyrazol-4-yl)pyrazine-2-carboxylate |
| TDH-421 | (R)-2-((4-fluorophenyl)amino)-2-oxo-1-phenylethyl 3-amino-6-(1-(1-(3-(1-((2-(2,6- |
| | dioxopiperidin-3-yl)-1,3-dioxoisoindolin-4-yl)glycyl)piperidin-4-yl)propanoyl)piperidin-4-yl)- |
| | 1H-pyrazol-4-yl)pyrazine-2-carboxylate |
| TDH-422 | (R)-2-((4-fluorophenyl)amino)-2-oxo-1-phenylethyl 3-amino-6-(1-(1-(3-(1-((2-(2,6- |
| | dioxopiperidin-3-yl)-1,3-dioxoisoindolin-5-yl)glycyl)piperidin-4-yl)propanoyl)piperidin-4-yl)- |
| | 1H-pyrazol-4-yl)pyrazine-2-carboxylate |
| TDH-423 | (R)-2-((4-fluorophenyl)amino)-2-oxo-1-phenylethyl 3-amino-6-(1-(1-(8-(2-(2,6- |
| | dioxopiperidin-3-yl)-1-oxoisoindolin-4-yl)oct-7-ynoyl)piperidin-4-yl)-1H-pyrazol-4- |
| | yl)pyrazine-2-carboxylate |
| TDH-425 | (R)-2-((4-fluorophenyl)amino)-2-oxo-1-phenylethyl 3-amino-6-(1-(1-((1-((1-(2-((2-(2,6- |
| | dioxopiperidin-3-yl)-1,3-dioxoisoindolin-5-yl)oxy)acetyl)piperidin-4-yl)methyl)piperidin-4- |
| | yl)methyl)piperidin-4-yl)-1H-pyrazol-4-yl)pyrazine-2-carboxylate |
| TDH-426 | (R)-2-((4-fluorophenyl)amino)-2-oxo-1-phenylethyl 3-amino-6-(1-(1-((1-((1-((2-(2,6- |
| | dioxopiperidin-3-yl)-1,3-dioxoisoindolin-5-yl)glycyl)piperidin-4-yl)methyl)piperidin-4- |
| | yl)methyl)piperidin-4-yl)-1H-pyrazol-4-yl)pyrazine-2-carboxylate |
| TDH-427 | (R)-2-((4-fluorophenyl)amino)-2-oxo-1-phenylethyl 3-amino-6-(1-(1-(3-(1-(1-(2-(2,6- |
| | dioxopiperidin-3-yl)-1,3-dioxoisoindolin-4-yl)azetidine-3-carbonyl)piperidin-4- |
| | yl)propanoyl)piperidin-4-yl)-1H-pyrazol-4-yl)pyrazine-2-carboxylate |
| TDH-428 | (R)-2-((4-fluorophenyl)amino)-2-oxo-1-phenylethyl 3-amino-6-(1-(1-(3-(1-(1-(2-(2,6- |
| | dioxopiperidin-3-yl)-1,3-dioxoisoindolin-5-yl)azetidine-3-carbonyl)piperidin-4- |
| | yl)propanoyl)piperidin-4-yl)-1H-pyrazol-4-yl)pyrazine-2-carboxylate |
| TDH-429 | (R)-2-((4-fluorophenyl)amino)-2-oxo-1-phenylethyl 3-amino-6-(1-(1-(3-(1-(2-(1-(2-(2,6- |
| | dioxopiperidin-3-yl)-1,3-dioxoisoindolin-4-yl)piperidin-4-yl)acetyl)piperidin-4- |
| | yl)propanoyl)piperidin-4-yl)-1H-pyrazol-4-yl)pyrazine-2-carboxylate |
| TDH-430 | (R)-2-((4-fluorophenyl)amino)-2-oxo-1-phenylethyl 3-amino-6-(1-(1-(3-(1-(2-(1-(2-(2,6- |
| | dioxopiperidin-3-yl)-1,3-dioxoisoindolin-5-yl)piperidin-4-yl)acetyl)piperidin-4- |
| | yl)propanoyl)piperidin-4-yl)-1H-pyrazol-4-yl)pyrazine-2-carboxylate |
| TDH-431 | (R)-2-((4-fluorophenyl)amino)-2-oxo-1-phenylethyl 3-amino-6-(1-(1-(3-(1-(3-(2-(2,6- |
| | dioxopiperidin-3-yl)-1,3-dioxoisoindolin-4-yl)propanoyl)piperidin-4-yl)propanoyl)piperidin-4- |
| | yl)-1H-pyrazol-4-yl)pyrazine-2-carboxylate |
| TDH-432 | (R)-2-((4-fluorophenyl)amino)-2-oxo-1-phenylethyl 3-amino-6-(1-(1-(3-(1-(3-(2-(2,6- |
| | dioxopiperidin-3-yl)-1,3-dioxoisoindolin-5-yl)propanoyl)piperidin-4-yl)propanoyl)piperidin-4- |
| | yl)-1H-pyrazol-4-yl)pyrazine-2-carboxylate |
| TDH-433 | (R)-2-((4-fluorophenyl)amino)-2-oxo-1-phenylethyl 3-amino-6-(1-(1-(3-(1-((3-(2,6- |
| | dioxopiperidin-3-yl)-4-oxo-3,4-dihydrobenzo[d][1,2,3]triazin-5-yl)glycyl)piperidin-4- |
| | yl)propanoyl)piperidin-4-yl)-1H-pyrazol-4-yl)pyrazine-2-carboxylate |
| TDH-434 | (R)-2-((4-fluorophenyl)amino)-2-oxo-1-phenylethyl 3-amino-6-(1-(1-(3-(1-((3-(2,6- |
| | dioxopiperidin-3-yl)-4-oxo-3,4-dihydrobenzo[d][1,2,3]triazin-6-yl)glycyl)piperidin-4- |
| | yl)propanoyl)piperidin-4-yl)-1H-pyrazol-4-yl)pyrazine-2-carboxylate |
| TDH-435 | (R)-2-((4-fluorophenyl)amino)-2-oxo-1-phenylethyl 3-amino-6-(1-(1-(3-(1-(1-(3-(2,6- |
| | dioxopiperidin-3-yl)-4-oxo-3,4-dihydrobenzo[d][1,2,3]triazin-5-yl)piperidine-4- |
| | carbonyl)piperidin-4-yl)propanoyl)piperidin-4-yl)-1H-pyrazol-4-yl)pyrazine-2-carboxylate |
| TDH-436 | (R)-2-((4-fluorophenyl)amino)-2-oxo-1-phenylethyl 3-amino-6-(1-(1-(3-(1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)propanoyl)piperidin-4-yl)-1H-pyrazol-4-yl)pyrazine-2-carboxylate |
| TDH-437 | (R)-2-((4-fluorophenyl)amino)-2-oxo-1-phenylethyl 3-amino-6-(1-(1-(3-(1-(2-((3-(2,6- |
| | dioxopiperidin-3-yl)-4-oxo-3,4-dihydrobenzo[d][1,2,3]triazin-6-yl)oxy)acetyl)piperidin-4- |
| | yl)propanoyl)piperidin-4-yl)-1H-pyrazol-4-yl)pyrazine-2-carboxylate |
| TDH-438 | (R)-2-((4-fluorophenyl)amino)-2-oxo-1-phenylethyl 3-amino-6-(1-(1-(3-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)propanoyl)piperidin-4-yl)-1H-pyrazol-4-yl)pyrazine-2-carboxylate |
| TDH-439 | (R)-2-((4-fluorophenyl)amino)-2-oxo-1-phenylethyl 3-amino-6-(1-(1-(3-(1-(3-(2-(2,6- |
| | dioxopiperidin-3-yl)-1-oxoisoindolin-4-yl)propanoyl)piperidin-4-yl)propanoyl)piperidin-4-yl)- |
| | 1H-pyrazol-4-yl)pyrazine-2-carboxylate |
| TDH-440 | (R)-2-((4-fluorophenyl)amino)-2-oxo-1-phenylethyl 3-amino-6-(1-(1-(3-(4-((1-(2-((2-(2,6- |
| | dioxopiperidin-3-yl)-1,3-dioxoisoindolin-5-yl)oxy)acetyl)piperidin-4-yl)methyl)piperazin-1- |
| | yl)propyl)piperidin-4-yl)-1H-pyrazol-4-yl)pyrazine-2-carboxylate |
| TDH-441 | (R)-2-((4-fluorophenyl)amino)-2-oxo-1-phenylethyl 3-amino-6-(1-(1-(3-(4-((1-((2-(2,6- |
| | dioxopiperidin-3-yl)-1,3-dioxoisoindolin-5-yl)glycyl)piperidin-4-yl)methyl)piperazin-1- |
| | yl)propyl)piperidin-4-yl)-1H-pyrazol-4-yl)pyrazine-2-carboxylate |
| TDH-442 | (R)-2-((4-fluorophenyl)amino)-2-oxo-1-phenylethyl 3-amino-6-(1-(1-(7-((1-(2-((2-(2,6- |
| | dioxopiperidin-3-yl)-1,3-dioxoisoindolin-5-yl)oxy)acetyl)piperidin-4-yl)methyl)-7- |
| | azaspiro[3.5]nonan-2-yl)piperidin-4-yl)-1H-pyrazol-4-yl)pyrazine-2-carboxylate |
| TDH-443 | (R)-2-((4-fluorophenyl)amino)-2-oxo-1-phenylethyl 3-amino-6-(1-(1-(7-((1-((2-(2,6- |
| | dioxopiperidin-3-yl)-1,3-dioxoisoindolin-5-yl)glycyl)piperidin-4-yl)methyl)-7- |
| | azaspiro[3.5]nonan-2-yl)piperidin-4-yl)-1H-pyrazol-4-yl)pyrazine-2-carboxylate |
| TDH-445 | (R)-2-((4-fluorophenyl)amino)-2-oxo-1-phenylethyl 3-amino-6-(1-(1-((1-(3-(2,4- |
| | dioxotetrahydropyrimidin-1(2H)-yl)-4-methoxybenzoyl)piperidin-4-yl)methyl)piperidin-4-yl)- |
| | 1H-pyrazol-4-yl)pyrazine-2-carboxylate |
| TDH-446 | (R)-2-((4-fluorophenyl)amino)-2-oxo-1-phenylethyl 3-amino-6-(1-(1-(2-(1-(3-(2,4- |
| | dioxotetrahydropyrimidin-1(2H)-yl)-4-methoxybenzoyl)piperidin-4-yl)ethyl)piperidin-4-yl)- |
| | 1H-pyrazol-4-yl)pyrazine-2-carboxylate |
| TDH-447 | (R)-2-((4-fluorophenyl)amino)-2-oxo-1-phenylethyl 3-amino-6-(1-(1-(3-(4-(3-(2,4- |
| | dioxotetrahydropyrimidin-1(2H)-yl)-4-methoxybenzoyl)piperazin-1-yl)propyl)piperidin-4-yl)- |
| | 1H-pyrazol-4-yl)pyrazine-2-carboxylate |
| TDH-448 | (R)-2-((4-fluorophenyl)amino)-2-oxo-1-phenylethyl 3-amino-6-(1-(1-(2-((1-(3-(2,4- |
| | dioxotetrahydropyrimidin-1(2H)-yl)-4-methoxybenzoyl)piperidin-4-yl)oxy)ethyl)piperidin-4- |
| | yl)-1H-pyrazol-4-yl)pyrazine-2-carboxylate |
| TDH-450 | (R)-2-((4-fluorophenyl)amino)-2-oxo-1-phenylethyl 3-amino-6-(1-(1-((1-(2-(1-(2-(1-methyl- |
| | 2,6-dioxopiperidin-3-yl)-1,3-dioxoisoindolin-5-yl)piperidin-4-yl)acetyl)piperidin-4- |
| | yl)methyl)piperidin-4-yl)-1H-pyrazol-4-yl)pyrazine-2-carboxylate |
| TDH-451 | (R)-2-((4-fluorophenyl)amino)-2-oxo-1-phenylethyl 3-amino-6-(1-(1-(2-(1-(1-(2-((2-(2,6- |
| | dioxopiperidin-3-yl)-1,3-dioxoisoindolin-5-yl)oxy)acetyl)piperidin-4-yl)-1H-1,2,3-triazol-4- |
| | yl)ethyl)piperidin-4-yl)-1H-pyrazol-4-yl)pyrazine-2-carboxylate |
| TDH-452 | (R)-2-((4-fluorophenyl)amino)-2-oxo-1-phenylethyl 3-amino-6-(1-(1-(2-(1-(1-((2-(2,6- |
| | dioxopiperidin-3-yl)-1,3-dioxoisoindolin-5-yl)glycyl)piperidin-4-yl)-1H-1,2,3-triazol-4- |
| | yl)ethyl)piperidin-4-yl)-1H-pyrazol-4-yl)pyrazine-2-carboxylate |
| TDH-453 | (R)-2-((4-fluorophenyl)amino)-2-oxo-1-phenylethyl 3-amino-6-(1-(1-(3-(5-(2-((2-(2,6- |
| | dioxopiperidin-3-yl)-1,3-dioxoisoindolin-5-yl)oxy)acetyl)-2,5-diazabicyclo[2.2.1]heptan-2- |
| | yl)propyl)piperidin-4-yl)-1H-pyrazol-4-yl)pyrazine-2-carboxylate |
| TDH-454 | (R)-2-((4-fluorophenyl)amino)-2-oxo-1-phenylethyl 3-amino-6-(1-(1-(3-(5-((2-(2,6- |
| | dioxopiperidin-3-yl)-1,3-dioxoisoindolin-5-yl)glycyl)-2,5-diazabicyclo[2.2.1]heptan-2- |
| | yl)propyl)piperidin-4-yl)-1H-pyrazol-4-yl)pyrazine-2-carboxylate |
| TDH-466 | (R)-2-((4-fluorophenyl)amino)-2-oxo-1-phenylethyl 3-amino-6-(1-(1-(3-(5-(2-((2-(2,6- |
| | dioxopiperidin-3-yl)-1,3-dioxoisoindolin-5-yl)oxy)acetyl)hexahydropyrrolo[3,4-c]pyrrol- |
| | 2(1H)-yl)propyl)piperidin-4-yl)-1H-pyrazol-4-yl)pyrazine-2-carboxylate |
| TDH-467 | (R)-2-((4-fluorophenyl)amino)-2-oxo-1-phenylethyl 3-amino-6-(1-(1-(3-(5-((2-(2,6- |
| | dioxopiperidin-3-yl)-1,3-dioxoisoindolin-5-yl)glycyl)hexahydropyrrolo[3,4-c]pyrrol-2(1H)- |
| | yl)propyl)piperidin-4-yl)-1H-pyrazol-4-yl)pyrazine-2-carboxylate |
| TDH-472 | (R)-2-((4-fluorophenyl)amino)-2-oxo-1-phenylethyl 3-amino-6-(1-(1-(7-((1-(2-(2,6- |
| | dioxopiperidin-3-yl)-1,3-dioxoisoindolin-5-yl)piperidin-4-yl)methyl)-7-azaspiro[3.5]nonan-2- |
| | yl)piperidin-4-yl)-1H-pyrazol-4-yl)pyrazine-2-carboxylate |
| TDH-473 | (R)-2-((4-fluorophenyl)amino)-2-oxo-1-phenylethyl 3-amino-6-(1-(1-((1-((1-(2-(2,6- |
| | dioxopiperidin-3-yl)-1,3-dioxoisoindolin-5-yl)piperidin-4-yl)methyl)piperidin-4- |
| | yl)methyl)piperidin-4-yl)-1H-pyrazol-4-yl)pyrazine-2-carboxylate |
| TDH-474 | (R)-2-((4-fluorophenyl)amino)-2-oxo-1-phenylethyl 3-amino-6-(1-(1-(3-(4-((1-(2-(2,6- |
| | dioxopiperidin-3-yl)-1,3-dioxoisoindolin-5-yl)piperidin-4-yl)methyl)piperazin-1- |
| | yl)propyl)piperidin-4-yl)-1H-pyrazol-4-yl)pyrazine-2-carboxylate |
| TDH-475 | (R)-2-((4-fluorophenyl)amino)-2-oxo-1-phenylethyl 3-amino-6-(1-(1-(2-(1-(3-(2-(2,6- |
| | dioxopiperidin-3-yl)-1-oxoisoindolin-5-yl)propanoyl)piperidin-4-yl)ethyl)piperidin-4-yl)-1H- |
| | pyrazol-4-yl)pyrazine-2-carboxylate |
| TDH-476 | (R)-2-((4-fluorophenyl)amino)-2-oxo-1-phenylethyl 3-amino-6-(1-(1-(3-(4-(3-(2-(2,6- |
| | dioxopiperidin-3-yl)-1-oxoisoindolin-5-yl)propanoyl)piperazin-1-yl)propyl)piperidin-4-yl)-1H- |
| | pyrazol-4-yl)pyrazine-2-carboxylate |
| TDH-477 | (R)-2-((4-fluorophenyl)amino)-2-oxo-1-phenylethyl 3-amino-6-(1-(1-((1-((1-(3-(2,4- |
| | dioxotetrahydropyrimidin-1(2H)-yl)-4-methoxybenzoyl)piperidin-4-yl)methyl)piperidin-4- |
| | yl)methyl)piperidin-4-yl)-1H-pyrazol-4-yl)pyrazine-2-carboxylate |
| TDH-478 | (R)-2-((4-fluorophenyl)amino)-2-oxo-1-phenylethyl 3-amino-6-(1-(1-(3-(4-((1-(3-(2,4- |
| | dioxotetrahydropyrimidin-1(2H)-yl)-4-methoxybenzoyl)piperidin-4-yl)methyl)piperazin-1- |
| | yl)propyl)piperidin-4-yl)-1H-pyrazol-4-yl)pyrazine-2-carboxylate |
| TDH-480 | (R)-2-((4-fluorophenyl)amino)-2-oxo-1-phenylethyl 3-amino-6-(1-(1-(3-(5-(3-(2-(2,6- |
| | dioxopiperidin-3-yl)-1,3-dioxoisoindolin-5-yl)propanoyl)hexahydropyrrolo[3,4-c]pyrrol- |
| | 2(1H)-yl)propyl)piperidin-4-yl)-1H-pyrazol-4-yl)pyrazine-2-carboxylate |
| TDH-481 | (R)-2-((4-fluorophenyl)amino)-2-oxo-1-phenylethyl 3-amino-6-(1-(1-(3-(1-(2-(2,6- |
| | dioxopiperidin-3-yl)-1,3-dioxoisoindolin-5-yl)piperidin-4-yl)propyl)piperidin-4-yl)-1H- |
| | pyrazol-4-yl)pyrazine-2-carboxylate |
| TDH-482 | (R)-2-((4-fluorophenyl)amino)-2-oxo-1-phenylethyl 3-amino-6-(1-(1-(3-(1-(2-((2-(2,6- |
| | dioxopiperidin-3-yl)-1,3-dioxoisoindolin-5-yl)oxy)acetyl)piperidin-4-yl)propyl)piperidin-4-yl)- |
| | 1H-pyrazol-4-yl)pyrazine-2-carboxylate |
| TDH-483 | (R)-2-((4-fluorophenyl)amino)-2-oxo-1-phenylethyl 3-amino-6-(1-(1-(3-(1-((2-(2,6- |
| | dioxopiperidin-3-yl)-1,3-dioxoisoindolin-5-yl)glycyl)piperidin-4-yl)propyl)piperidin-4-yl)-1H- |
| | pyrazol-4-yl)pyrazine-2-carboxylate |
| TDH-484 | (R)-2-((4-fluorophenyl)amino)-2-oxo-1-phenylethyl 3-amino-6-(1-(1-(3-(1-(3-(2-(2,6- |
| | dioxopiperidin-3-yl)-1,3-dioxoisoindolin-5-yl)propanoyl)piperidin-4-yl)propyl)piperidin-4-yl)- |
| | 1H-pyrazol-4-yl)pyrazine-2-carboxylate |
| TDH-485 | (R)-2-((3-chlorophenyl)amino)-2-oxo-1-phenylethyl 3-amino-6-(1-(1-(3-(4-(2-((2-(2,6- |
| | dioxopiperidin-3-yl)-1,3-dioxoisoindolin-5-yl)oxy)acetyl)piperazin-1-yl)propyl)piperidin-4- |
| | yl)-1H-pyrazol-4-yl)pyrazine-2-carboxylate |
| TDH-486 | (R)-2-((3-methoxyphenyl)amino)-2-oxo-1-phenylethyl 3-amino-6-(1-(1-(3-(4-(2-((2-(2,6- |
| | dioxopiperidin-3-yl)-1,3-dioxoisoindolin-5-yl)oxy)acetyl)piperazin-1-yl)propyl)piperidin-4- |
| | yl)-1H-pyrazol-4-yl)pyrazine-2-carboxylate |
| TDH-487 | (R)-2-oxo-1-phenyl-2-(m-tolylamino)ethyl 3-amino-6-(1-(1-(3-(4-(2-((2-(2,6-dioxopiperidin- |
| | 3-yl)-1,3-dioxoisoindolin-5-yl)oxy)acetyl)piperazin-1-yl)propyl)piperidin-4-yl)-1H-pyrazol-4- |
| | yl)pyrazine-2-carboxylate |
| TDH-491 | 3-amino-6-(1-(1-(3-(4-(2-(2,6-dioxopiperidin-3-yl)-1,3-dioxoisoindolin-5-yl)piperazin-1- |
| | yl)propyl)piperidin-4-yl)-1H-pyrazol-4-yl)-N-((R)-2-((4-fluorophenyl)amino)-2-oxo-1- |
| | phenylethyl)pyrazine-2-carboxamide |
| TDH-492 | 3-amino-6-(1-(1-(3-(4-(2-((2-(2,6-dioxopiperidin-3-yl)-1,3-dioxoisoindolin-5- |
| | yl)oxy)acetyl)piperazin-1-yl)propyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-493 | 3-amino-6-(1-(1-(3-(4-((2-(2,6-dioxopiperidin-3-yl)-1,3-dioxoisoindolin-5-yl)glycyl)piperazin- |
| | 1-yl)propyl)piperidin-4-yl)-1H-pyrazol-4-yl)-N-((R)-2-((4-fluorophenyl)amino)-2-oxo-1- |
| | phenylethyl)pyrazine-2-carboxamide |
| TDH-494 | (R)-1-(2-chlorophenyl)-2-((4-fluorophenyl)amino)-2-oxoethyl 3-amino-6-(1-(1-(3-(4-(2-((2- |
| | (2,6-dioxopiperidin-3-yl)-1,3-dioxoisoindolin-5-yl)oxy)acetyl)piperazin-1-yl)propyl)piperidin- |
| | 4-yl)-1H-pyrazol-4-yl)pyrazine-2-carboxylate |
| TDH-494 | (R)-1-(2-chlorophenyl)-2-((4-fluorophenyl)amino)-2-oxoethyl 3-amino-6-(1-(1-(3-(4-(2-((2- |
| | (2,6-dioxopiperidin-3-yl)-1,3-dioxoisoindolin-5-yl)oxy)acetyl)piperazin-1-yl)propyl)piperidin- |
| | 4-yl)-1H-pyrazol-4-yl)pyrazine-2-carboxylate |
| TDH-495 | (R)-2-((4-fluorophenyl)amino)-2-oxo-1-phenylethyl 3-amino-6-(1-(1-(3-(4-(2-(3-((1-(2,6- |
| | dioxopiperidin-3-yl)-2,5-dioxo-2,5-dihydro-1H-pyrrol-3-yl)amino)phenyl)acetyl)piperazin-1- |
| | yl)propyl)piperidin-4-yl)-1H-pyrazol-4-yl)pyrazine-2-carboxylate |
| TDH-496 | (R)-2-((4-fluorophenyl)amino)-2-oxo-1-phenylethyl 3-amino-6-(1-(1-(2-((1-(2-(3-((1-(2,6- |
| | dioxopiperidin-3-yl)-2,5-dioxo-2,5-dihydro-1H-pyrrol-3-yl)amino)phenyl)acetyl)piperidin-4- |
| | yl)oxy)ethyl)piperidin-4-yl)-1H-pyrazol-4-yl)pyrazine-2-carboxylate |
| TDH-497 | (R)-2-((4-fluorophenyl)amino)-2-oxo-1-phenylethyl 3-amino-6-(1-(1-(((1r,4r)-4-((3-(2,4- |
| | dioxotetrahydropyrimidin-1(2H)-yl)-4- |
| | methoxybenzamido)methyl)cyclohexyl)methyl)piperidin-4-yl)-1H-pyrazol-4-yl)pyrazine-2- |
| | carboxylate |
| TDH-498 | (R)-2-((4-fluorophenyl)amino)-2-oxo-1-phenylethyl 3-amino-6-(1-(1-(((1r,4r)-4-((2-(3-((1- |
| | (2,6-dioxopiperidin-3-yl)-2,5-dioxo-2,5-dihydro-1H-pyrrol-3- |
| | yl)amino)phenyl)acetamido)methyl)cyclohexyl)methyl)piperidin-4-yl)-1H-pyrazol-4- |
| | yl)pyrazine-2-carboxylate |
| TDH-499 | (R)-2-((4-fluorophenyl)amino)-2-oxo-1-phenylethyl 3-amino-6-(1-(1-(7-((1-(3-(2,4- |
| | dioxotetrahydropyrimidin-1(2H)-yl)-4-methoxybenzoyl)piperidin-4-yl)methyl)-7- |
| | azaspiro[3.5]nonan-2-yl)piperidin-4-yl)-1H-pyrazol-4-yl)pyrazine-2-carboxylate |
| TDH-500 | (R)-2-((4-fluorophenyl)amino)-2-oxo-1-phenylethyl 3-amino-6-(1-(1-(7-((1-(2-(3-((1-(2,6- |
| | dioxopiperidin-3-yl)-2,5-dioxo-2,5-dihydro-1H-pyrrol-3-yl)amino)phenyl)acetyl)piperidin-4- |
| | yl)methyl)-7-azaspiro[3.5]nonan-2-yl)piperidin-4-yl)-1H-pyrazol-4-yl)pyrazine-2- |
| | carboxylate |
| TDH-501 | (R)-2-((4-fluorophenyl)amino)-2-oxo-1-phenylethyl 3-amino-6-(1-(1-((1-(2-(1-(3-(2,4- |
| | dioxotetrahydropyrimidin-1(2H)-yl)-4-methoxybenzoyl)piperidin-4-yl)ethyl)piperidin-4- |
| | yl)methyl)piperidin-4-yl)-1H-pyrazol-4-yl)pyrazine-2-carboxylate |
| TDH-502 | (R)-2-((4-fluorophenyl)amino)-2-oxo-1-phenylethyl 3-amino-6-(1-(1-((1-(2-(1-(1-(3-(2,4- |
| | dioxotetrahydropyrimidin-1(2H)-yl)-4-methoxybenzoyl)piperidine-4-carbonyl)piperidin-4- |
| | yl)ethyl)piperidin-4-yl)methyl)piperidin-4-yl)-1H-pyrazol-4-yl)pyrazine-2-carboxylate |
| TDH-503 | (R)-2-((4-fluorophenyl)amino)-2-oxo-1-phenylethyl 3-amino-6-(1-(1-((1-(7-(3-(2,4- |
| | dioxotetrahydropyrimidin-1(2H)-yl)-4-methoxybenzoyl)-7-azaspiro[3.5]nonan-2- |
| | yl)piperidin-4-yl)methyl)piperidin-4-yl)-1H-pyrazol-4-yl)pyrazine-2-carboxylate |
| TDH-504 | (R)-2-((4-fluorophenyl)amino)-2-oxo-1-phenylethyl 3-amino-6-(1-(1-(2-(1-(7-(3-(2,4- |
| | dioxotetrahydropyrimidin-1(2H)-yl)-4-methoxybenzoyl)-7-azaspiro[3.5]nonan-2- |
| | yl)piperidin-4-yl)ethyl)piperidin-4-yl)-1H-pyrazol-4-yl)pyrazine-2-carboxylate |
| TDH-505 | (R)-2-((4-fluorophenyl)amino)-2-oxo-1-phenylethyl 3-amino-6-(1-(1-(2-(1-((1-(3-(2,4- |
| | dioxotetrahydropyrimidin-1(2H)-yl)-4-methoxybenzoyl)piperidin-4-yl)methyl)piperidin-4- |
| | yl)ethyl)piperidin-4-yl)-1H-pyrazol-4-yl)pyrazine-2-carboxylate |
| TDH-506 | (R)-2-((4-fluorophenyl)amino)-2-oxo-1-phenylethyl 3-amino-6-(1-(1-((1-(2-((1-(3-(2,4- |
| | dioxotetrahydropyrimidin-1(2H)-yl)-4-methoxybenzoyl)piperidin-4-yl)oxy)ethyl)piperidin-4- |
| | yl)methyl)piperidin-4-yl)-1H-pyrazol-4-yl)pyrazine-2-carboxylate |
| TDH-507 | (R)-2-((4-fluorophenyl)amino)-2-oxo-1-phenylethyl 3-amino-6-(1-(1-((1-(2-(1-(2-(3-((1-(2,6- |
| | dioxopiperidin-3-yl)-2,5-dioxo-2,5-dihydro-1H-pyrrol-3-yl)amino)phenyl)acetyl)piperidin-4- |
| | yl)ethyl)piperidin-4-yl)methyl)piperidin-4-yl)-1H-pyrazol-4-yl)pyrazine-2-carboxylate |
| TDH-508 | (R)-2-((4-fluorophenyl)amino)-2-oxo-1-phenylethyl 3-amino-6-(1-(1-((1-(7-(1-(3-(2,4- |
| | dioxotetrahydropyrimidin-1(2H)-yl)-4-methoxybenzoyl)piperidine-4-carbonyl)-7- |
| | azaspiro[3.5]nonan-2-yl)piperidin-4-yl)methyl)piperidin-4-yl)-1H-pyrazol-4-yl)pyrazine-2- |
| | carboxylate |
| TDH-509 | (R)-2-((4-fluorophenyl)amino)-2-oxo-1-phenylethyl 3-amino-6-(1-(1-(2-(1-(7-(2-(3-((1-(2,6- |
| | dioxopiperidin-3-yl)-2,5-dioxo-2,5-dihydro-1H-pyrrol-3-yl)amino)phenyl)acetyl)-7- |
| | azaspiro[3.5]nonan-2-yl)piperidin-4-yl)ethyl)piperidin-4-yl)-1H-pyrazol-4-yl)pyrazine-2- |
| | carboxylate |
| TDH-510 | (R)-2-((4-fluorophenyl)amino)-2-oxo-1-phenylethyl 3-amino-6-(1-(1-(2-(1-((1-(2-(3-((1-(2,6- |
| | dioxopiperidin-3-yl)-2,5-dioxo-2,5-dihydro-1H-pyrrol-3-yl)amino)phenyl)acetyl)piperidin-4- |
| | yl)methyl)piperidin-4-yl)ethyl)piperidin-4-yl)-1H-pyrazol-4-yl)pyrazine-2-carboxylate |
| TDH-511 | (R)-2-((4-fluorophenyl)amino)-2-oxo-1-phenylethyl 3-amino-6-(1-(1-(2-(1-((1-(1-(3-(2,4- |
| | dioxotetrahydropyrimidin-1(2H)-yl)-4-methoxybenzoyl)piperidine-4-carbonyl)piperidin-4- |
| | yl)methyl)piperidin-4-yl)ethyl)piperidin-4-yl)-1H-pyrazol-4-yl)pyrazine-2-carboxylate |
| TDH-512 | (R)-2-((4-fluorophenyl)amino)-2-oxo-1-phenylethyl 3-amino-6-(1-(1-((1-(2-((1-(1-(3-(2,4- |
| | dioxotetrahydropyrimidin-1(2H)-yl)-4-methoxybenzoyl)piperidine-4-carbonyl)piperidin-4- |
| | yl)oxy)ethyl)piperidin-4-yl)methyl)piperidin-4-yl)-1H-pyrazol-4-yl)pyrazine-2-carboxylate |
| TDH-513 | (R)-2-((4-fluorophenyl)amino)-2-oxo-1-phenylethyl 3-amino-6-(1-(1-((1-(2-(1-(2-(2,6- |
| | dioxopiperidin-3-yl)-6-fluoro-1,3-dioxoisoindolin-5-yl)piperidin-4-yl)ethyl)piperidin-4- |
| | yl)methyl)piperidin-4-yl)-1H-pyrazol-4-yl)pyrazine-2-carboxylate |
| TDH-514 | (R)-2-((4-fluorophenyl)amino)-2-oxo-1-phenylethyl 3-amino-6-(1-(1-(2-(1-((1-(2-(2,6- |
| | dioxopiperidin-3-yl)-6-fluoro-1,3-dioxoisoindolin-5-yl)piperidin-4-yl)methyl)piperidin-4- |
| | yl)ethyl)piperidin-4-yl)-1H-pyrazol-4-yl)pyrazine-2-carboxylate |
| TDH-515 | (R)-2-((4-fluorophenyl)amino)-2-oxo-1-phenylethyl 3-amino-6-(1-(1-((1-(1-(3-(2,4- |
| | dioxotetrahydropyrimidin-1(2H)-yl)-4-methoxybenzoyl)piperidine-4-carbonyl)piperidin-4- |
| | yl)methyl)piperidin-4-yl)-1H-pyrazol-4-yl)pyrazine-2-carboxylate |
| TDH-516 | (R)-2-((4-fluorophenyl)amino)-2-oxo-1-phenylethyl 3-amino-6-(1-(1-((1-(2-(3-((1-(2,6- |
| | dioxopiperidin-3-yl)-2,5-dioxo-2,5-dihydro-1H-pyrrol-3-yl)amino)phenyl)acetyl)piperidin-4- |
| | yl)methyl)piperidin-4-yl)-1H-pyrazol-4-yl)pyrazine-2-carboxylate |
| TDH-517 | (R)-2-((4-fluorophenyl)amino)-2-oxo-1-phenylethyl 3-amino-6-(1-(1-(2-(1-(1-(3-(2,4- |
| | dioxotetrahydropyrimidin-1(2H)-yl)-4-methoxybenzoyl)piperidine-4-carbonyl)piperidin-4- |
| | yl)ethyl)piperidin-4-yl)-1H-pyrazol-4-yl)pyrazine-2-carboxylate |
| TDH-518 | (R)-2-((4-fluorophenyl)amino)-2-oxo-1-phenylethyl 3-amino-6-(1-(1-(2-(1-(2-(3-((1-(2,6- |
| | dioxopiperidin-3-yl)-2,5-dioxo-2,5-dihydro-1H-pyrrol-3-yl)amino)phenyl)acetyl)piperidin-4- |
| | yl)ethyl)piperidin-4-yl)-1H-pyrazol-4-yl)pyrazine-2-carboxylate |
| TDH-519 | (R)-2-((4-fluorophenyl)amino)-2-oxo-1-phenylethyl 3-amino-6-(1-(1-((1-(2-(2,6- |
| | dioxopiperidin-3-yl)-6-fluoro-1,3-dioxoisoindolin-5-yl)piperidin-4-yl)methyl)piperidin-4-yl)- |
| | 1H-pyrazol-4-yl)pyrazine-2-carboxylate |
| TDH-520 | (R)-2-((4-fluorophenyl)amino)-2-oxo-1-phenylethyl 3-amino-6-(1-(1-(2-(1-(2-(2,6- |
| | dioxopiperidin-3-yl)-6-fluoro-1,3-dioxoisoindolin-5-yl)piperidin-4-yl)ethyl)piperidin-4-yl)-1H- |
| | pyrazol-4-yl)pyrazine-2-carboxylate |
| TDH-521 | (R)-2-((4-fluorophenyl)amino)-2-oxo-1-phenylethyl 3-amino-6-(1-(1-((1-(1-(2,6- |
| | dioxopiperidin-3-yl)-2,5-dioxo-2,5-dihydro-1H-pyrrol-3-yl)piperidin-4-yl)methyl)piperidin-4- |
| | yl)-1H-pyrazol-4-yl)pyrazine-2-carboxylate |
| TDH-522 | (R)-2-((4-fluorophenyl)amino)-2-oxo-1-phenylethyl 3-amino-6-(1-(1-(2-(1-(1-(2,6- |
| | dioxopiperidin-3-yl)-2,5-dioxo-2,5-dihydro-1H-pyrrol-3-yl)piperidin-4-yl)ethyl)piperidin-4-yl)- |
| | 1H-pyrazol-4-yl)pyrazine-2-carboxylate |
| TDH-523 | (R)-2-((4-fluorophenyl)amino)-2-oxo-1-phenylethyl 3-amino-6-(1-(1-((1-(1-(1-(2,6- |
| | dioxopiperidin-3-yl)-2,5-dioxo-2,5-dihydro-1H-pyrrol-3-yl)piperidine-4-carbonyl)piperidin-4- |
| | yl)methyl)piperidin-4-yl)-1H-pyrazol-4-yl)pyrazine-2-carboxylate |
| TDH-524 | (R)-2-((4-fluorophenyl)amino)-2-oxo-1-phenylethyl 3-amino-6-(1-(1-(2-(1-(1-(1-(2,6- |
| | dioxopiperidin-3-yl)-2,5-dioxo-2,5-dihydro-1H-pyrrol-3-yl)piperidine-4-carbonyl)piperidin-4- |
| | yl)ethyl)piperidin-4-yl)-1H-pyrazol-4-yl)pyrazine-2-carboxylate |
| TDH-525 | (R)-2-((4-fluorophenyl)amino)-2-oxo-1-phenylethyl 3-amino-6-(1-(1-(3-(4-(2-((2-(1-methyl- |
| | 2,6-dioxopiperidin-3-yl)-1,3-dioxoisoindolin-5-yl)oxy)acetyl)piperazin-1-yl)propyl)piperidin- |
| | 4-yl)-1H-pyrazol-4-yl)pyrazine-2-carboxylate |
| TDH-526 | (R)-2-((4-fluorophenyl)amino)-2-oxo-1-phenylethyl 3-amino-6-(1-(1-(3-(4-(1-(1-(2,6- |
| | dioxopiperidin-3-yl)-2,5-dioxo-2,5-dihydro-1H-pyrrol-3-yl)piperidine-4-carbonyl)piperazin-1- |
| | yl)propyl)piperidin-4-yl)-1H-pyrazol-4-yl)pyrazine-2-carboxylate |
| TDH-527 | (R)-2-((4-fluorophenyl)amino)-2-oxo-1-phenylethyl 3-amino-6-(1-(1-((1-((1-(1-(1-(2,6- |
| | dioxopiperidin-3-yl)-2,5-dioxo-2,5-dihydro-1H-pyrrol-3-yl)piperidine-4-carbonyl)piperidin-4- |
| | yl)methyl)piperidin-4-yl)methyl)piperidin-4-yl)-1H-pyrazol-4-yl)pyrazine-2-carboxylate |
| TDH-528 | (R)-2-((4-fluorophenyl)amino)-2-oxo-1-phenylethyl 3-amino-6-(1-(1-(7-((1-(1-(1-(2,6- |
| | dioxopiperidin-3-yl)-2,5-dioxo-2,5-dihydro-1H-pyrrol-3-yl)piperidine-4-carbonyl)piperidin-4- |
| | yl)methyl)-7-azaspiro[3.5]nonan-2-yl)piperidin-4-yl)-1H-pyrazol-4-yl)pyrazine-2- |
| | carboxylate |
| TDH-529 | (R)-2-((4-fluorophenyl)amino)-2-oxo-1-phenylethyl 3-amino-6-(1-(1-(2-((1-(1-(1-(2,6- |
| | dioxopiperidin-3-yl)-2,5-dioxo-2,5-dihydro-1H-pyrrol-3-yl)piperidine-4-carbonyl)piperidin-4- |
| | yl)oxy)ethyl)piperidin-4-yl)-1H-pyrazol-4-yl)pyrazine-2-carboxylate |
| TDH-530 | (R)-2-((4-fluorophenyl)amino)-2-oxo-1-phenylethyl 3-amino-6-(1-(1-(3-(4-(2-(2,6- |
| | dioxopiperidin-3-yl)-1-oxoisoindoline-5-carbonyl)piperazin-1-yl)propyl)piperidin-4-yl)-1H- |
| | pyrazol-4-yl)pyrazine-2-carboxylate |
| TDH-531 | (R)-2-((4-fluorophenyl)amino)-2-oxo-1-phenylethyl 3-amino-6-(1-(1-(2-(1-(1-(1-(2,6- |
| | dioxopiperidin-3-yl)-2,5-dioxo-2,5-dihydro-1H-pyrrol-3-yl)azetidine-3-carbonyl)piperidin-4- |
| | yl)ethyl)piperidin-4-yl)-1H-pyrazol-4-yl)pyrazine-2-carboxylate |
| TDH-532 | (R)-2-((4-fluorophenyl)amino)-2-oxo-1-phenylethyl 3-amino-6-(1-(1-(3-(4-(1-(1-(2,6- |
| | dioxopiperidin-3-yl)-2,5-dioxo-2,5-dihydro-1H-pyrrol-3-yl)azetidine-3-carbonyl)piperazin-1- |
| | yl)propyl)piperidin-4-yl)-1H-pyrazol-4-yl)pyrazine-2-carboxylate |
| TDH-533 | (R)-2-((4-fluorophenyl)amino)-2-oxo-1-phenylethyl 3-amino-6-(1-(1-((1-((1-(1-(1-(2,6- |
| | dioxopiperidin-3-yl)-2,5-dioxo-2,5-dihydro-1H-pyrrol-3-yl)azetidine-3-carbonyl)piperidin-4- |
| | yl)methyl)piperidin-4-yl)methyl)piperidin-4-yl)-1H-pyrazol-4-yl)pyrazine-2-carboxylate |
| TDH-534 | (R)-2-((4-fluorophenyl)amino)-2-oxo-1-phenylethyl 3-amino-6-(1-(1-(7-((1-(1-(1-(2,6- |
| | dioxopiperidin-3-yl)-2,5-dioxo-2,5-dihydro-1H-pyrrol-3-yl)azetidine-3-carbonyl)piperidin-4- |
| | yl)methyl)-7-azaspiro[3.5]nonan-2-yl)piperidin-4-yl)-1H-pyrazol-4-yl)pyrazine-2- |
| | carboxylate |
| TDH-535 | (R)-2-((4-fluorophenyl)amino)-2-oxo-1-phenylethyl 3-amino-6-(1-(1-(3-(4-(4-((2,6- |
| | dioxopiperidin-3-yl)carbamoyl)benzoyl)piperazin-1-yl)propyl)piperidin-4-yl)-1H-pyrazol-4- |
| | yl)pyrazine-2-carboxylate |
| TDH-536 | (R)-2-((4-fluorophenyl)amino)-2-oxo-1-phenylethyl 3-amino-6-(1-(1-(2-(1-(4-((2,6- |
| | dioxopiperidin-3-yl)carbamoyl)benzoyl)piperidin-4-yl)ethyl)piperidin-4-yl)-1H-pyrazol-4- |
| | yl)pyrazine-2-carboxylate |
| TDH-537 | (R)-2-((4-fluorophenyl)amino)-2-oxo-1-phenylethyl 3-amino-6-(1-(1-(7-((1-(4-((2,6- |
| | dioxopiperidin-3-yl)carbamoyl)benzoyl)piperidin-4-yl)methyl)-7-azaspiro[3.5]nonan-2- |
| | yl)piperidin-4-yl)-1H-pyrazol-4-yl)pyrazine-2-carboxylate |
| TDH-538 | (R)-2-((4-fluorophenyl)amino)-2-oxo-1-phenylethyl 3-amino-6-(1-(1-(2-((1-(4-((2,6- |
| | dioxopiperidin-3-yl)carbamoyl)benzoyl)piperidin-4-yl)oxy)ethyl)piperidin-4-yl)-1H-pyrazol- |
| | 4-yl)pyrazine-2-carboxylate |
| TDH-539 | (R)-2-((4-fluorophenyl)amino)-2-oxo-1-phenylethyl 3-amino-6-(1-(1-((1-((1-(4-((2,6- |
| | dioxopiperidin-3-yl)carbamoyl)benzoyl)piperidin-4-yl)methyl)piperidin-4- |
| | yl)methyl)piperidin-4-yl)-1H-pyrazol-4-yl)pyrazine-2-carboxylate |
| TDH-540 | (R)-2-((4-fluorophenyl)amino)-2-oxo-1-phenylethyl 3-amino-6-(1-(1-(3-(4-(1-(1-(2,6- |
| | dioxopiperidin-3-yl)-3-methyl-2-oxo-2,3-dihydro-1H-benzo[d]imidazol-5-yl)piperidine-4- |
| | carbonyl)piperazin-1-yl)propyl)piperidin-4-yl)-1H-pyrazol-4-yl)pyrazine-2-carboxylate |
| TDH-541 | (R)-2-((4-fluorophenyl)amino)-2-oxo-1-phenylethyl 3-amino-6-(1-(1-(2-((1-(1-(1-(2,6- |
| | dioxopiperidin-3-yl)-2,5-dioxo-2,5-dihydro-1H-pyrrol-3-yl)azetidine-3-carbonyl)piperidin-4- |
| | yl)oxy)ethyl)piperidin-4-yl)-1H-pyrazol-4-yl)pyrazine-2-carboxylate |
| TDH-542 | (R)-2-((4-fluorophenyl)amino)-2-oxo-1-phenylethyl 3-amino-6-(1-(1-(3-(4-(3-((2,6- |
| | dioxopiperidin-3-yl)carbamoyl)benzoyl)piperazin-1-yl)propyl)piperidin-4-yl)-1H-pyrazol-4- |
| | yl)pyrazine-2-carboxylate |
| TDH-543 | (R)-2-((4-fluorophenyl)amino)-2-oxo-1-phenylethyl 3-amino-6-(1-(1-(2-(1-(3-((2,6- |
| | dioxopiperidin-3-yl)carbamoyl)benzoyl)piperidin-4-yl)ethyl)piperidin-4-yl)-1H-pyrazol-4- |
| | yl)pyrazine-2-carboxylate |
| TDH-544 | (R)-2-((4-fluorophenyl)amino)-2-oxo-1-phenylethyl 3-amino-6-(1-(1-(7-((1-(3-((2,6- |
| | dioxopiperidin-3-yl)carbamoyl)benzoyl)piperidin-4-yl)methyl)-7-azaspiro[3.5]nonan-2- |
| | yl)piperidin-4-yl)-1H-pyrazol-4-yl)pyrazine-2-carboxylate |
| TDH-545 | (R)-2-((4-fluorophenyl)amino)-2-oxo-1-phenylethyl 3-amino-6-(1-(1-(2-((1-(3-((2,6- |
| | dioxopiperidin-3-yl)carbamoyl)benzoyl)piperidin-4-yl)oxy)ethyl)piperidin-4-yl)-1H-pyrazol- |
| | 4-yl)pyrazine-2-carboxylate |
| TDH-546 | (R)-2-((4-fluorophenyl)amino)-2-oxo-1-phenylethyl 3-amino-6-(1-(1-((1-((1-(3-((2,6- |
| | dioxopiperidin-3-yl)carbamoyl)benzoyl)piperidin-4-yl)methyl)piperidin-4- |
| | yl)methyl)piperidin-4-yl)-1H-pyrazol-4-yl)pyrazine-2-carboxylate |
| TDH-547 | (R)-2-((4-fluorophenyl)amino)-2-oxo-1-phenylethyl 3-amino-6-(1-(1-(2-(1-(2-(2,6- |
| | dioxopiperidin-3-yl)-1-oxoisoindoline-5-carbonyl)piperidin-4-yl)ethyl)piperidin-4-yl)-1H- |
| | pyrazol-4-yl)pyrazine-2-carboxylate |
| TDH-548 | (R)-2-((4-fluorophenyl)amino)-2-oxo-1-phenylethyl 3-amino-6-(1-(1-(7-((1-(2-(2,6- |
| | dioxopiperidin-3-yl)-1-oxoisoindoline-5-carbonyl)piperidin-4-yl)methyl)-7- |
| | azaspiro[3.5]nonan-2-yl)piperidin-4-yl)-1H-pyrazol-4-yl)pyrazine-2-carboxylate |
| TDH-549 | (R)-2-((4-fluorophenyl)amino)-2-oxo-1-phenylethyl 3-amino-6-(1-(1-(2-((1-(2-(2,6- |
| | dioxopiperidin-3-yl)-1-oxoisoindoline-5-carbonyl)piperidin-4-yl)oxy)ethyl)piperidin-4-yl)-1H- |
| | pyrazol-4-yl)pyrazine-2-carboxylate |
| TDH-550 | (R)-2-((4-fluorophenyl)amino)-2-oxo-1-phenylethyl 3-amino-6-(1-(1-((1-((1-(2-(2,6- |
| | dioxopiperidin-3-yl)-1-oxoisoindoline-5-carbonyl)piperidin-4-yl)methyl)piperidin-4- |
| | yl)methyl)piperidin-4-yl)-1H-pyrazol-4-yl)pyrazine-2-carboxylate |
| TDH-551 | (R)-2-((4-fluorophenyl)amino)-2-oxo-1-phenylethyl 3-amino-6-(1-(1-(3-(4-(3-(2,4- |
| | dioxotetrahydropyrimidin-1(2H)-yl)benzofuran-6-carbonyl)piperazin-1-yl)propyl)piperidin-4- |
| | yl)-1H-pyrazol-4-yl)pyrazine-2-carboxylate |
| TDH-552 | (R)-2-((4-fluorophenyl)amino)-2-oxo-1-phenylethyl 3-amino-6-(1-(1-(2-(1-(3-(2,4- |
| | dioxotetrahydropyrimidin-1(2H)-yl)benzofuran-6-carbonyl)piperidin-4-yl)ethyl)piperidin-4- |
| | yl)-1H-pyrazol-4-yl)pyrazine-2-carboxylate |
| TDH-553 | (R)-2-((4-fluorophenyl)amino)-2-oxo-1-phenylethyl 3-amino-6-(1-(1-(7-((1-(3-(2,4- |
| | dioxotetrahydropyrimidin-1(2H)-yl)benzofuran-6-carbonyl)piperidin-4-yl)methyl)-7- |
| | azaspiro[3.5]nonan-2-yl)piperidin-4-yl)-1H-pyrazol-4-yl)pyrazine-2-carboxylate |
| TDH-554 | (R)-2-((4-fluorophenyl)amino)-2-oxo-1-phenylethyl 3-amino-6-(1-(1-(2-((1-(3-(2,4- |
| | dioxotetrahydropyrimidin-1(2H)-yl)benzofuran-6-carbonyl)piperidin-4- |
| | yl)oxy)ethyl)piperidin-4-yl)-1H-pyrazol-4-yl)pyrazine-2-carboxylate |
| TDH-555 | (R)-2-((4-fluorophenyl)amino)-2-oxo-1-phenylethyl 3-amino-6-(1-(1-((1-((1-(3-(2,4- |
| | dioxotetrahydropyrimidin-1(2H)-yl)benzofuran-6-carbonyl)piperidin-4-yl)methyl)piperidin-4- |
| | yl)methyl)piperidin-4-yl)-1H-pyrazol-4-yl)pyrazine-2-carboxylate |

In another embodiment, the present invention provides a pharmaceutical composition comprising a therapeutically effective amount of a compound of Chemical Formula 1 or 2 or a pharmaceutically acceptable salt thereof according to the present invention, and a pharmaceutically acceptable carrier.

Another embodiment of the present invention provides a method for treating a disease or condition, comprising administering a therapeutically effective amount of a compound of Chemical Formula 1 or 2 or a pharmaceutically acceptable salt thereof to a subject in need thereof, wherein the disease or condition is traumatic brain injury, stroke, bone marrow failure, (acute) pancreatitis, arteriosclerosis, metabolic disease (e.g., diabetes, hypertension, hyperlipidemia, heart disease, etc.), ischemia-reperfusion injury, transplantation, infection, chronic obstructive pulmonary disease, remote lung injury, hepatotoxicity, (alcoholic and non-alcoholic) steatohepatitis, remote liver injury, autoimmune hepatitis, psoriasis, hypoxic ischemic encephalopathy, ovarian cancer, renal injury, fatty liver, inflammation, Crohn's colitis, ulcerative colitis, or ileitis. In another embodiment of the present invention, the disease or condition is nonalcoholic steatohepatitis, autoimmune hepatitis, psoriasis, hypoxic ischemic encephalopathy, stroke, ovarian cancer, renal damage, fatty liver or inflammation.

That is, the present invention provides a medical use characterized by using a compound of Chemical Formula 1 or 2 or a pharmaceutically acceptable salt thereof according to the present invention as an active ingredient. In one embodiment, the medical use of the present invention is for the treatment or prevention of a disease or condition described herein.

### Medical Uses and Methods of Treatment of the Compounds According to the Present Invention

The present invention further provides methods for treating a disease or condition in a subject having or susceptible to having such a disease or condition, by administering to the subject a therapeutically-effective amount of one or more compounds as described above. In one embodiment, the treatment is preventative treatment. In another embodiment, the treatment is palliative treatment. In another embodiment, the treatment is restorative treatment.

### 1. Diseases or Conditions

The compounds of the present invention for inhibiting MLKL activity are useful for various therapeutic or prophylactic applications. These compounds can be used to control or inhibit MLKL activity, and can also be used to inhibit MLKL-mediated necroptosis. It can also be used for the treatment of MLKL-related diseases or to prevent exacerbation of these diseases. Accordingly, the present invention provides a method for inhibiting, controlling, or degrading intracellular MLKL activity. In this method, the cells are contacted with an effective amount of a compound of the present invention. In one embodiment, the cell is within a subject. The method of the present invention comprises administering a pharmaceutical composition comprising a therapeutically or prophylactically effective amount of an MLKL activity inhibitor to a subject in need of treatment or prevention.

In one embodiment, the present invention provides a method for inhibiting, degrading or controlling MLKL activity in cells of an MLKL-associated disease. For example, the present invention can be used to inhibit MLKL activity in cells of traumatic brain injury, stroke, bone marrow failure, (acute) pancreatitis, atherosclerosis, metabolic diseases (e.g., diabetes, hypertension, hyperlipidemia, heart disease, etc.), ischemia-reperfusion injury, transplantation, infection, chronic obstructive pulmonary disease, remote lung injury, hepatotoxicity, (alcoholic and non-alcoholic) steatohepatitis, remote liver injury, autoimmune hepatitis, psoriasis, hypoxic ischemic encephalopathy, ovarian cancer, renal injury, fatty liver, inflammation, Crohn's colitis, ulcerative colitis, ileitis, etc. In another embodiment of the present invention, the present invention can be used to inhibit MLKL activity in cells of non-alcoholic steatohepatitis, autoimmune hepatitis, psoriasis, hypoxic ischemic encephalopathy, stroke, ovarian cancer, renal damage, fatty liver or inflammation.

A compound of the present invention can be administered to the subject in the form of a pharmaceutical composition described herein.

In another embodiment, the present invention provides a method for treating or preventing MLKL-related diseases in a subject, such MLKL-related diseases include traumatic brain injury, stroke, bone marrow failure, (acute) pancreatitis, atherosclerosis, metabolic disease, ischemia-reperfusion injury, transplantation, infection, chronic obstructive pulmonary disease, remote lung injury, hepatotoxicity, (alcoholic and non-alcoholic) steatohepatitis, remote liver injury, autoimmune hepatitis, psoriasis, hypoxic-ischemic encephalopathy, ovarian cancer, renal injury, fatty liver, inflammation, Crohn's colitis, ulcerative colitis, ileitis and the like. In another embodiment of the present invention, the MLKL-related disease is nonalcoholic steatohepatitis, autoimmune hepatitis, psoriasis, hypoxic ischemic encephalopathy, stroke, ovarian cancer, renal injury, fatty liver or inflammation. Such methods include administering to a subject in need of treatment an amount of a compound of the present invention sufficient to inhibit MLKL activity, i.e., a therapeutically effective amount.

### 2. Subjects

Suitable subjects to be treated according to the present invention include mammalian subjects. Mammals according to the present disclosure include, but are not limited to, human, canine, feline, bovine, caprine, equine, ovine, porcine, rodents, lagomorphs, primates, and the like, and encompass mammals in utero. Subjects can be of both sexes and can be at any stage of development.

In one embodiment, the suitable subject to be treated according to the present invention is human.

### 3. Administration and Dosing

The compounds of the present invention are generally administered in a therapeutically effective amount.

The compounds of the present invention can be administered by any suitable route in the form of a pharmaceutical composition adapted to such a route, and in a dose effective for the treatment intended. An effective dosage is typically in the range of about 0.001 to about 100 mg per kg body weight per day, preferably about 0.01 to about 50 mg/kg/day, in single or divided doses. Depending on age, species and disease or condition being treated, dosage levels below the lower limit of this range may be suitable. In other cases, still larger doses may be used without harmful side effects. Larger doses may also be divided into several smaller doses, for administration throughout the day.

### Pharmaceutical Compositions, Dosage Forms and Administration Routes

For the treatment of the diseases or conditions referred to above, the compounds described herein or pharmaceutically acceptable salts thereof can be administered as follows:

### Oral Administration

The compounds of the present invention may be administered orally, including by swallowing, so that the compound enters the gastrointestinal tract, or absorbed into the blood stream directly from the mouth (e.g., buccal or sublingual administration).

Suitable compositions for oral administration include solid, liquid, gel or powder formulations, and have a dosage form such as tablet, lozenge, capsule, granule or powder.

Compositions for oral administration may optionally be enteric coated and may exhibit delayed or sustained release through the enteric coating. That is, the composition for oral administration according to the present invention may be a formulation having an immediate or modified release pattern.

### Parenteral Administration

Compounds of the present disclosure may be administered directly into the blood stream, muscle, or internal organs. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods.

Compositions for parenteral administration may be formulated as immediate or modified release, including delayed or sustained release.

Most parenteral formulations are liquid compositions, and the liquid composition is an aqueous solution containing the active ingredient according to the present invention, a salt, a buffering agent, an isotonic agent, and the like.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions.

### Topical Administration

Compounds of the present invention may be administered topically to the skin or transdermally. Formulations for this topical administration can include lotions, solutions, creams, gels, hydrogels, ointments, foams, implants, patches and the like. Pharmaceutically acceptable carriers for topical administration formulations can include water, alcohol, mineral oil, glycerin, polyethylene glycol and the like. Topical administration can also be performed by electroporation, iontophoresis, phonophoresis and the like.

Compositions for topical administration may be formulated as immediate or modified release, including delayed or sustained release.

### Advantageous Effects

The present invention provides compounds capable of exhibiting various pharmacological activities by inhibiting or degrading MLKL activity, pharmaceutical compositions containing them as an active ingredient, their medical uses (especially for treatment or prevention of traumatic brain injury, stroke, bone marrow failure, (acute) pancreatitis, arteriosclerosis, ischemia-reperfusion injury, transplant, infection, chronic obstructive pulmonary disease, remote lung injury, hepatotoxicity, (alcoholic and non-alcoholic) steatohepatitis, remote liver injury, autoimmune hepatitis, psoriasis, hypoxic ischemic encephalopathy, ovarian cancer, kidney damage, fatty liver, inflammation, Crohn's colitis, ulcerative colitis, ileitis, etc.), and a treatment method comprising administering them to a subject in need of treatment or prevention. The compounds or pharmaceutically acceptable salts thereof according to the present invention are excellent in various aspects such as safety and stability, and have high selectivity in terms of MLKL activity inhibition or degradation, and thus can exhibit excellent medicinal effects.

### BRIEF DESCRIPTION OF FIGURES

FIG. 1 is a diagram showing the necroptosis signaling mechanism associated with MLKL.

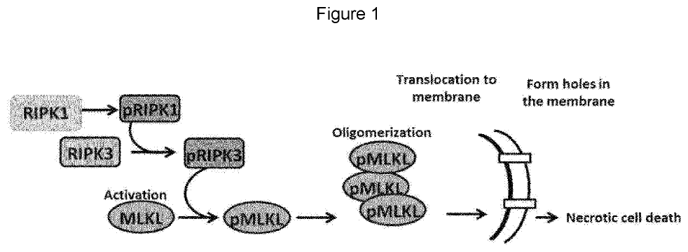

### MODE FOR INVENTION

Hereinafter, the present invention is described in considerable detail with examples to help those skilled in the art understand the present invention. However, the following examples are offered by way of illustration and are not intended to limit the scope of the invention. It is apparent that various changes may be made without departing from the spirit and scope of the invention or sacrificing all of its material advantages.

### Preparation of Compounds of the Present Invention

Hereinafter, the synthesis process of some compounds of the present invention will be described, and the other compounds not mentioned below can be prepared by substituting starting materials, intermediates and/or reactants in a similar manner.

### Synthesis of TDH-069 and TDH-097

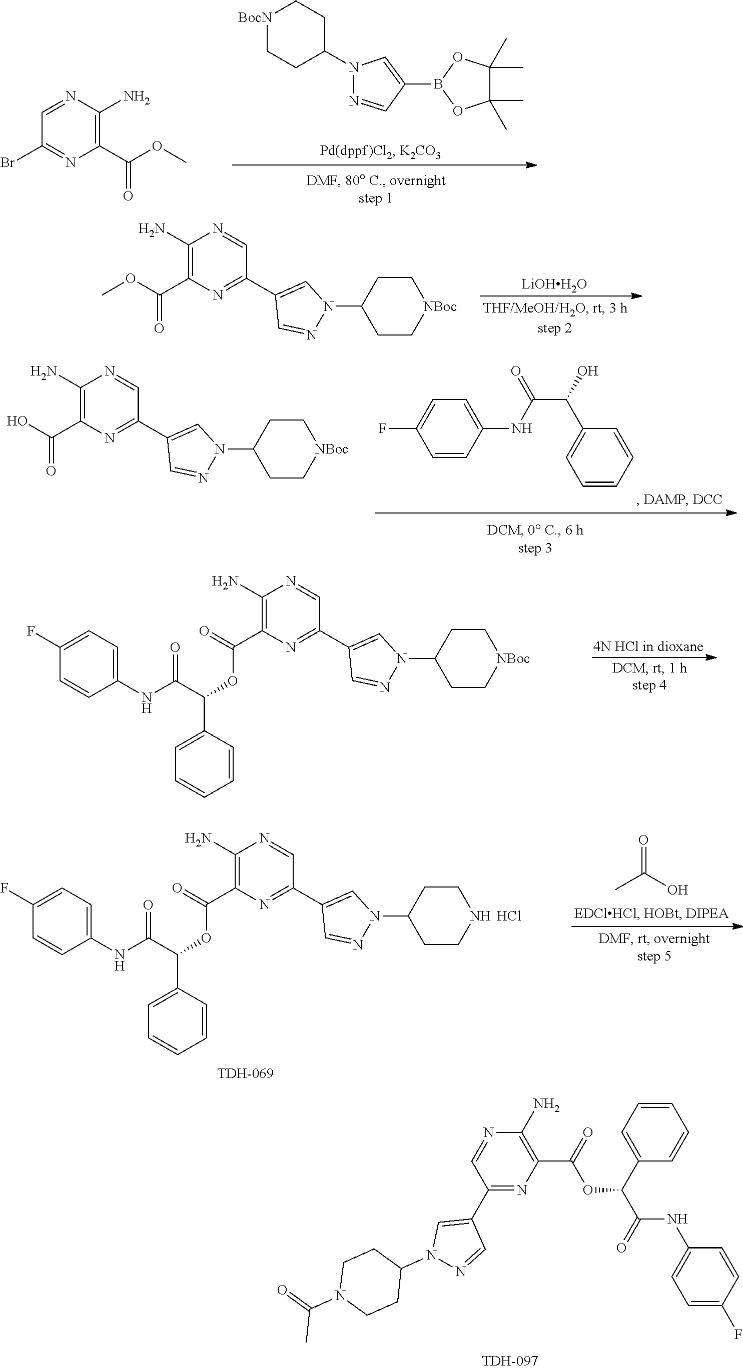

### Synthesis of methyl 3-amino-6-(1-(1-(tert-butoxycarbonyl)piperidin-4-yl)-1H-pyrazol-4-yl)pyrazine-2-carboxylate (step 1)

Methyl 3-amino-6-bromopyrazine-2-carboxylate (3.0 g, 13 mmol, 1 eq.) was dissolved in DMF (50 mL), and Pd(dppf)Cl₂ (946 mg, 1.3 mmol, 0.1 eq.), tert-butyl 4-(4-(4,4,5,5-tetramethyl-1,3,2-dioxaborolan-2-yl)-1H-pyrazol-1-yl)piperidine-1-carboxylate (7.3 g, 19 mmol, 1.5 eq.), and potassium carbonate (7.1 g, 52 mmol, 4.0 eq.) were added and reacted overnight at 80° C. in the presence of nitrogen. After completion of the reaction, the reactant was concentrated under reduced pressure to remove DMF, diluted with ethyl acetate, and extracted with water. The combined organic layer was washed with brine, and the residual water was removed through anhydrous Na₂SO₄, followed by filtration. After concentrating the organic layer, the desired methyl 3-amino-6-(1-(1-(tert-butoxycarbonyl)piperidin-4-yl)-1H-pyrazol-4-yl)pyrazine-2-carboxylate (4.5 g, 86%) was obtained.

¹H NMR (300 MHz, CDCl₃) δ 8.41 (s, 1H), 7.90 (s, 1H), 7.87 (s, 1H), 6.48 (s, 2H), 4.31-4.16 (m, 3H), 3.94 (s, 3H), 2.86 (t, J=12.7 Hz, 2H), 2.17-2.07 (m, 2H), 1.98-1.84 (m, 2H), 1.44 (s, 9H).

### Synthesis of 3-amino-6-(1-(1-(tert-butoxycarbonyl)piperidin-4-yl)-1H-pyrazol-4-yl)pyrazine-2-carboxylic acid (step 2)

Methyl 3-amino-6-(1-(1-(tert-butoxycarbonyl)piperidin-4-yl)-1H-pyrazol-4-yl)pyrazine-2-carboxylate (4.5 g, 11 mmol, 1 eq.) was dissolved in THF/MeOH/H₂O (40 mL/10 mL/10 mL), and then LiOH·H₂O (2.3 g, 55 mmol, 5.0 eq.) was added and reacted at room temperature for 3 hours. After completion of the reaction, the reaction solvent was concentrated, diluted with EtOAc, and extracted with water. The aqueous layer was adjusted to pH 4-5 with 1 N HCl aqueous solution and then extracted with EtOAc. The combined organic layer was washed with brine, and the residual water was removed through anhydrous Na₂SO₄, followed by filtration. The organic layer was concentrated under reduced pressure to obtain the desired 3-amino-6-(1-(1-(tert-butoxycarbonyl)piperidin-4-yl)-1H-pyrazol-4-yl)pyrazine-2-carboxylic acid (4.1 g, 96%) was obtained.

¹H NMR (300 MHz, DMSO-d₆) δ 8.57 (s, 1H), 8.39 (s, 1H), 8.03 (s, 1H), 7.41 (s, 2H), 4.47-4.28 (m, 1H), 4.13-3.97 (m, 2H), 3.07-2.82 (m, 2H), 2.13-1.97 (m, 2H), 1.88-1.64 (m, 2H), 1.43 (s, 9H).

### Synthesis of (R)—N-(4-Fluorophenyl)-2-hydroxy-2-phenylacetamide

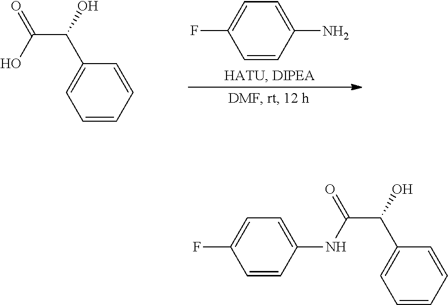 [SMILES: O=C(O)[C@H](O)c1ccccc1 ; Nc1ccc(F)cc1 ; O=C(Nc1ccc(F)cc1)[C@H](O)c1ccccc1]

After dissolving (R)-2-hydroxy-2-phenylacetic acid (10 g, 65 mmol, 1 eq.) in DMF (150 mL), 4-fluoroanilin (8.0 mL, 85 mmol, 1.3 eq.), HATU (37 g, 98 mmol, 1.5 eq.), and DIPEA (57 mL, 328 mmol, 5.0 eq.) were added and reacted at room temperature for 12 hours. After completion of the reaction, DMF was concentrated under reduced pressure, diluted with EA, and washed with water. The combined organic layer was washed with brine, and the residual water was removed through anhydrous Na₂SO₄, followed by filtration. After concentrating the organic layer, column chromatography was used to obtain the desired (R)—N-(4-Fluorophenyl)-2-hydroxy-2-phenylacetamide (11 g, 69%).

¹H NMR (300 MHz, DMSO-d₆) δ 10.02 (s, 1H), 7.79-7.68 (m, 2H), 7.57-7.47 (m, 2H), 7.43-7.27 (m, 4H), 7.17-7.08 (m, 2H), 6.49-6.43 (m, 1H), 5.13-5.07 (m, 1H).

### Synthesis of (R)-2-((4-Fluorophenyl)amino)-2-oxo-1-phenylethyl 3-amino-6-(1-(1-(tert-butoxycarbonyl)piperidin-4-yl)-1H-pyrazol-4-yl)pyrazine-2-carboxylate (step 3)

3-Amino-6-(1-(1-(tert-butoxycarbonyl)piperidin-4-yl)-1H-pyrazol-4-yl)pyrazine-2-carboxylic acid (1.5 g, 3.9 mmol, 1 eq.) was dissolved in DCM (15 mL), and (R)—N-(4-Fluorophenyl)-2-hydroxy-2-phenylacetamide (0.85 g, 3.9 mmol, 1 eq.), DCC (800 mg, 3.9 mmol, 1 eq.) and DMAP (236 mg, 1.9 mmol, 0.5 eq.) were added and reacted at room temperature for 6 hours. After completion of the reaction, it was diluted with DCM and washed with water. The combined organic layer was washed with brine, and the residual water was removed through anhydrous Na₂SO₄, followed by filtration. After concentrating the organic layer, column chromatography was used to obtain the desired (R)-2-((4-Fluorophenyl)amino)-2-oxo-1-phenylethyl 3-amino-6-(1-(1-(tert-butoxycarbonyl)piperidin-4-yl)-1H-pyrazol-4-yl)pyrazine-2-carboxylate (1.6 g, 67%).

¹H NMR (400 MHz, CDCl₃) δ 8.94 (s, 1H), 8.53 (s, 1H), 8.01 (s, 1H), 7.90 (s, 1H), 7.68-7.64 (m, 2H), 7.61-7.53 (m, 2H), 7.46-7.41 (m, 3H), 7.03 (t, J=8.6 Hz, 2H), 6.46 (s, 1H), 6.35 (s, 2H), 4.41-4.21 (m, 5H), 2.97-2.90 (m, 2H), 2.20-2.16 (m, 2H), 1.63 (s, 9H).

### Synthesis of (R)-2-((4-Fluorophenyl)amino)-2-oxo-1-phenylethyl 3-amino-6-(1-(piperidin-4-yl)-1H-pyrazol-4-yl)pyrazine-2-carboxylate hydrochloride (TDH-069, step 4)

(R)-2-((4-Fluorophenyl)amino)-2-oxo-1-phenylethyl 3-amino-6-(1-(1-(tert-butoxycarbonyl)piperidin-4-yl)-1H-pyrazol-4-yl)pyrazine-2-carboxylate (1.6 g, 2.5 mmol) was dissolved in DCM (15 mL), 4N HCl in dioxane (5 mL) was slowly added, and the mixture was stirred at room temperature for 1 hour. After completion of the reaction, the precipitate was filtered to obtain the desired (R)-2-((4-Fluorophenyl)amino)-2-oxo-1-phenylethyl 3-amino-6-(1-(piperidin-4-yl)-1H-pyrazol-4-yl)pyrazine-2-carboxylate hydrochloride (1.6 g).

### Synthesis of (R)-2-((4-fluorophenyl)amino)-2-oxo-1-phenylethyl 6-(1-(1-acetylpiperidin-4-yl)-1H-pyrazol-4-yl)-3-aminopyrazine-2-carboxylate (TDH-097, step 5)

Acetic acid (0.002 mL, 0.036 mmol, 1 eq.) was dissolved in DMF (0.15 mL), and EDCI·HCl (7.6 mg, 0.040 mmol, 1.1 eq.), HOBt (6.0 mg, 0.040 mmol, 1 eq.), and DIPEA (0.031 mL, 0.18 mmol, 5 eq.) were added. Then, (R)-2-((4-Fluorophenyl)amino)-2-oxo-1-phenylethyl 3-amino-6-(1-(piperidin-4-yl)-1H-pyrazol-4-yl)pyrazine-2-carboxylate hydrochloride (20 mg, 0.036 mmol, 1 eq.) was added and reacted overnight at room temperature. After completion of the reaction, the mixture was diluted with ethyl acetate and extracted with water. The combined organic layer was washed with brine, and the residual water was removed through anhydrous Na₂SO₄, followed by filtration. After concentrating the organic layer, column chromatography was performed to obtain (R)-2-((4-fluorophenyl)amino)-2-oxo-1-phenylethyl 6-(1-(1-acetylpiperidin-4-yl)-1H-pyrazol-4-yl)-3-aminopyrazine-2-carboxylate (12 mg, 60%).

### Synthesis of TDH-066 and TDH-070

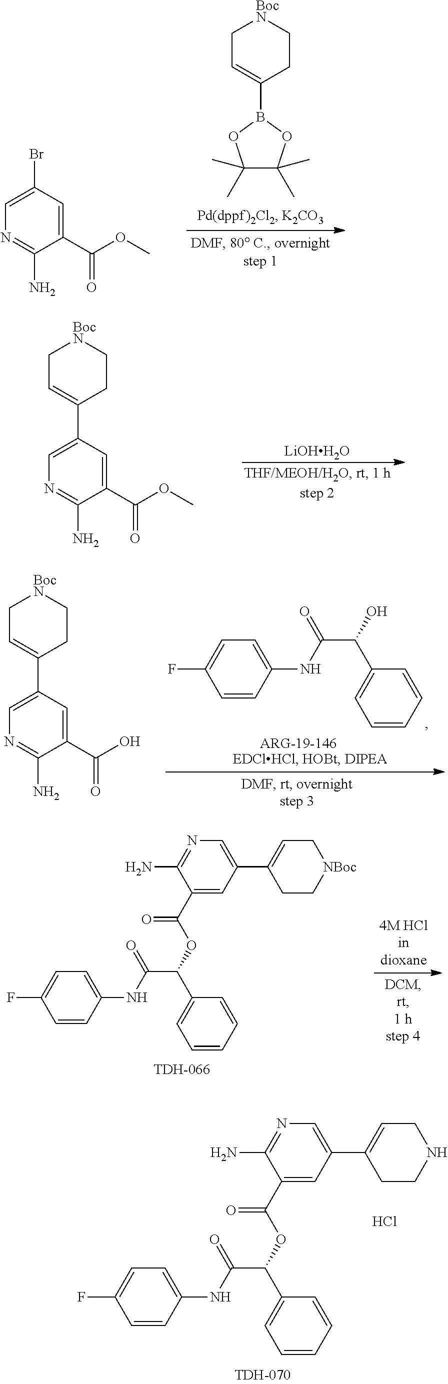

### Synthesis of 1′-(tert-Butyl) 5-methyl 6-amino-3′,6′-dihydro-[3,4′-bipyridine]-1′,5(2′H)-dicarboxylate (step 1)

Methyl 2-amino-5-bromonicotinate (500 mg, 2.16 mmol, 1.0 eq.), tert-butyl 4-(4,4,5,5-tetramethyl-1,3,2-dioxaborolan-2-yl)-3,6-dihydropyridine-1(2H)-carboxylate (1.0 g, 3.25 mmol, 1.5 eq.), K₂CO₃ (1.2 g, 8.66 mmol, 4.0 eq.), and Pd(dppf)₂Cl₂ (158 mg, 0.22 mmol, 0.1 eq.) were dissolved in DMF (10 mL) and reacted overnight at 80° C. in the presence of nitrogen. After completion of the reaction, DMF was concentrated under reduced pressure to remove, diluted with ethyl acetate, and extracted with water. The combined organic layer was washed with brine, and the residual water was removed through anhydrous Na₂SO₄, followed by filtration. After concentrating the organic layer, column chromatography was performed to obtain the desired 1′-(tert-Butyl) 5-methyl 6-amino-3′,6′-dihydro-[3,4′-bipyridine]-1′,5(2′H)-dicarboxylate (587 mg, 82%).

¹H NMR (400 MHz, CDCl₃) δ 8.29 (d, J=2.5 Hz, 1H), 8.12 (d, J=2.4 Hz, 1H), 5.95 (s, 1H), 4.12-4.01 (m, 2H), 3.94-3.87 (m, 3H), 3.69-3.59 (m, 2H), 2.52-2.42 (m, 2H), 1.49 (s, 9H).

### Synthesis of 6-Amino-1′-(tert-butoxycarbonyl)-1′,2′,3′,6′-tetrahydro-[3,4′-bipyridine]-5-carboxylic acid (step 2)

1′-(tert-Butyl) 5-methyl 6-amino-3′,6′-dihydro-[3,4′-bipyridine]-1′,5(2′H)-dicarboxylate (587 mg, 1.8 mmol, 1 eq.) was dissolved in THF/MeOH/H₂O (8 mL/4 mL/4 mL), and LiOH·H₂O (369 mg, 8.8 mmol, 5 eq.) was added and reacted at room temperature for 1 hour. After completion of the reaction, the reaction solvent was concentrated, diluted with EtOAc, and extracted with water. The collected water layer was adjusted to pH 4-5 with 1 N HCl aqueous solution and then extracted with EtOAc. The combined organic layer was washed with brine, and the residual water was removed through anhydrous Na₂SO₄, followed by filtration. The organic layer was concentrated under reduced pressure to obtain the desired 6-amino-1′-(tert-butoxycarbonyl)-1′,2′,3′,6′-tetrahydro-[3,4′-bipyridine]-5-carboxylic acid (585 mg).

¹H NMR (300 MHz, MeOD) δ 8.38 (s, 1H), 8.16 (s, 1H), 6.10 (s, 1H), 4.16-4.03 (m, 2H), 3.73-3.61 (m, 2H), 2.59-2.44 (m, 2H), 1.51 (s, 9H).

### (R)-1′-(tert-Butyl) 5-(2-((4-fluorophenyl)amino)-2-oxo-1-phenylethyl) 6-amino-3′,6′-dihydro-[3,4′-bipyridine]-1′,5(2′H)-dicarboxylate (TDH-066, step 3)

6-Amino-1′-(tert-butoxycarbonyl)-1′,2′,3′,6′-tetrahydro-[3,4′-bipyridine]-5-carboxylic acid (100 mg, 0.31 mmol, 1 eq.), (R)—N-(4-Fluorophenyl)-2-hydroxy-2-phenylacetamide (77 mg, 0.31 mmol, 1 eq.), EDCI-HCl (66 mg, 0.34 mmol, 1.1 eq.), HOBt (53 mg, 0.34 mmol, 1.1 eq.), and DIPEA (0.27 mL, 1.6 mmol, 5 eq.) were dissolved in DMF (1.5 mL) and reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain the desired (R)-1′-(tert-Butyl) 5-(2-((4-fluorophenyl)amino)-2-oxo-1-phenylethyl) 6-amino-3′,6′-dihydro-[3,4′-bipyridine]-1′,5(2′H)-dicarboxylate (35 mg, 21%).

### (R)-2-((4-Fluorophenyl)amino)-2-oxo-1-phenylethyl 6-amino-1′,2′,3′,6′-tetrahydro-[3,4′-bipyridine]-5-carboxylate hydrochloride (TDH-070, step 4)

(R)-1′-(tert-Butyl) 5-(2-((4-fluorophenyl)amino)-2-oxo-1-phenylethyl) 6-amino-3′,6′-dihydro-[3,4 Dissolve′-bipyridine]-1′,5(2′H)-dicarboxylate (20 mg, 0.036 mmol) was dissolved in DCM (0.5 mL). 4N HCl in dioxane (0.5 mL) was slowly added thereto, and reacted at room temperature for 1 hour. After completion of the reaction, the precipitate was filtered to obtain the desired ARG-18-507 (12 mg), which was used in the next reaction without purification.

### Synthesis of TDH-098 and TDH-099

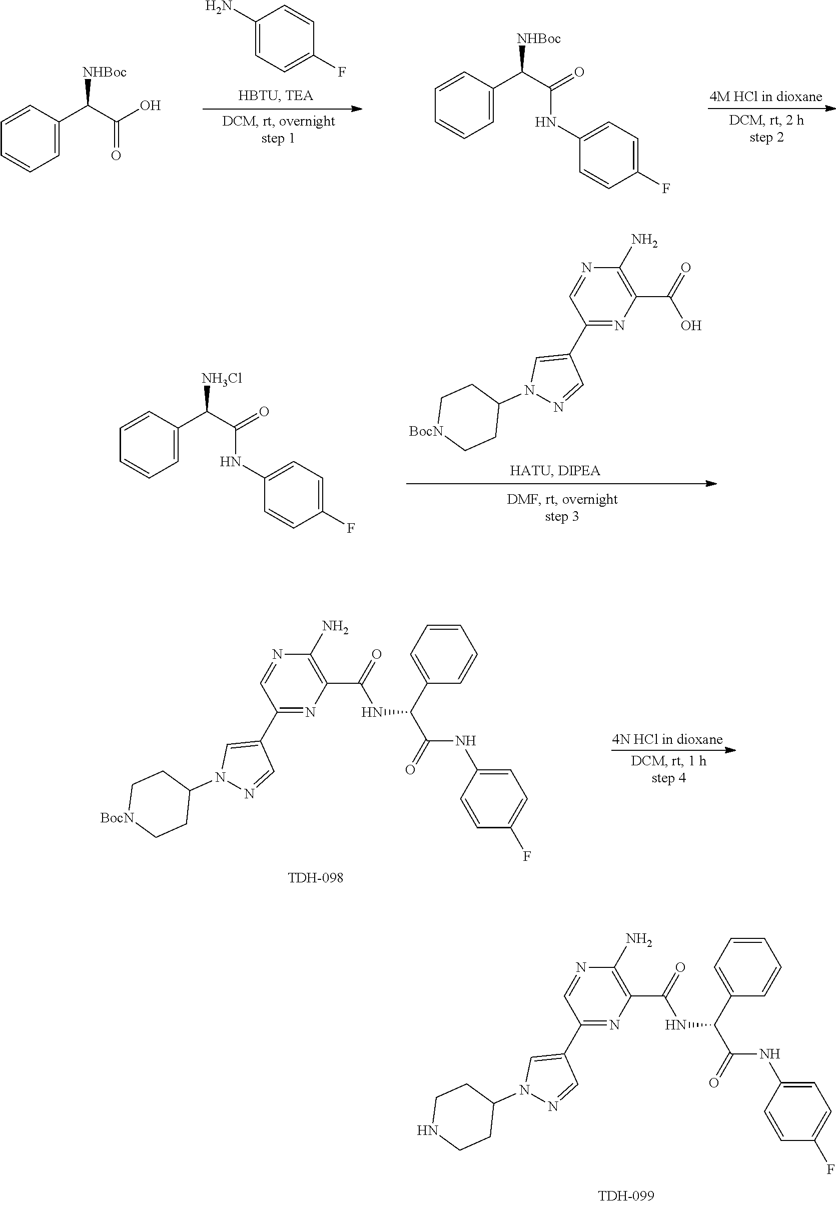

### Synthesis of tert-Butyl (R)-(2-((4-fluorophenyl)amino)-2-oxo-1-phenylethyl)carbamate (step 1)

(R)-2-((tert-Butoxycarbonyl)amino)-2-phenylacetic acid (1.00 g, 3.979 mmol) was dissolved in DCM (40 mL), and TEA (0.61 mL, 5.5 mmol) and HBTU (1.66 g, 4.377 mmol) were was added and stirred for 20 minutes. 4-Fluoroaniline (0.37 mL, 3.97 mmol) was added and reacted overnight at room temperature. The reaction solvent was concentrated, diluted with EtOAc, washed with saturated NH₄Cl aqueous solution, dried over anhydrous MgSO₄, and filtered. The collected organic solvents were concentrated and purified by column chromatography to obtain the desired white solid, tert-butyl (R)-(2-((4-fluorophenyl)amino)-2-oxo-1-phenylethyl)carbamate (915 mg, 2.65 mmol, 66%).

¹H NMR (300 MHz, CDCl₃) δ 8.02 (s, 1H), 7.45 (d, J=6.6 Hz, 2H), 7.41-7.32 (m, 2H), 6.98-6.89 (m, 2H), 5.78 (s, 1H), 5.36 (s, 1H), 1.43 (s, 9H).

### Synthesis of (R)-2-Amino-N-(4-fluorophenyl)-2-phenylacetamide (step 2)

Tert-butyl (R)-(2-((4-fluorophenyl)amino)-2-oxo-1-phenylethyl)carbamate (915 mg, 2.65 mmol) was dissolved in DCM (10.0 mL). 4M HCl in dioxane (5.0 mL) was added and reacted at room temperature for 2 hours. The reaction solvent was concentrated to obtain (R)-2-Amino-N-(4-fluorophenyl)-2-phenylacetamide (880 mg, crude) as a white solid.

¹H NMR (300 MHz, DMSO-d₆) δ 11.21 (s, 1H), 8.87 (s, 3H), 7.70-7.62 (m, 4H), 7.51-7.42 (m, 3H), 7.22-7.14 (m, 2H), 5.26 (s, 1H).

### Synthesis of tert-Butyl (R)-4-(4-(5-amino-6-((2-((4-fluorophenyl)amino)-2-oxo-1-phenylethyl)carbamoyl)pyrazin-2-yl)-1H-pyrazol-1-yl)piperidine-1-carboxylate (TDH-098, step 3)

After dissolving 3-amino-6-(1-(1-(tert-butoxycarbonyl)piperidin-4-yl)-1H-pyrazol-4-yl)pyrazine-2-carboxylic acid (300 mg, 0.772 mmol) in DMF, (R)-2-amino-N-(4-fluorophenyl)-2-phenylacetamide (216 mg, 0.772 mmol), HATU (440 mg, 1.15 mmol), and DIPEA (0.293 mL, 1.68 mmol) were added and reacted at room temperature overnight. After completion of the reaction, the mixture was diluted with EtOAc, washed with water, dried over anhydrous MgSO₄, and filtered. The organic solvent was concentrated under reduced pressure and purified by column chromatography to obtain the desired yellow solid, tert-butyl (R)-4-(4-(5-amino-6-((2-((4-fluorophenyl)amino)-2-oxo-1-phenylethyl)carbamoyl)pyrazin-2-yl)-1H-pyrazol-1-yl)piperidine-1-carboxylate (427 mg, 0.694 mmol, 89%).

### Synthesis of (R)-3-Amino-N-(2-((4-fluorophenyl)amino)-2-oxo-1-phenylethyl)-6-(1-(piperidin-4-yl)-1H-pyrazol-4-yl)pyrazine-2-carboxamide (TDH-099, step 4)

Tert-butyl (R)-4-(4-(5-amino-6-((2-((4-fluorophenyl)amino)-2-oxo-1-phenylethyl)carbamoyl)pyrazin-2-yl)-1H-pyrazol-1-yl)piperidine-1-carboxylate (315 mg, 0.514 mmol) was dissolved in DCM (4 mL), 4 M HCl in dioxane (2 mL) was added, and reacted at room temperature for 1 hour. After completion of the reaction, it was concentrated. After neutralizing with sat. NaHCO₃ solution, the precipitate was filtered and washed with water. The filtered water layer was extracted with EtOAc, concentrated, and collected with the precipitate to obtain (R)-3-Amino-N-(2-((4-fluorophenyl)amino)-2-oxo-1-phenylethyl)-6-(1-(piperidin-4-yl)-1H-pyrazol-4-yl)pyrazine-2-carboxamide (208 mg, 0.404 mmol, 78%) as a yellow solid.

### Synthesis of TDH-112

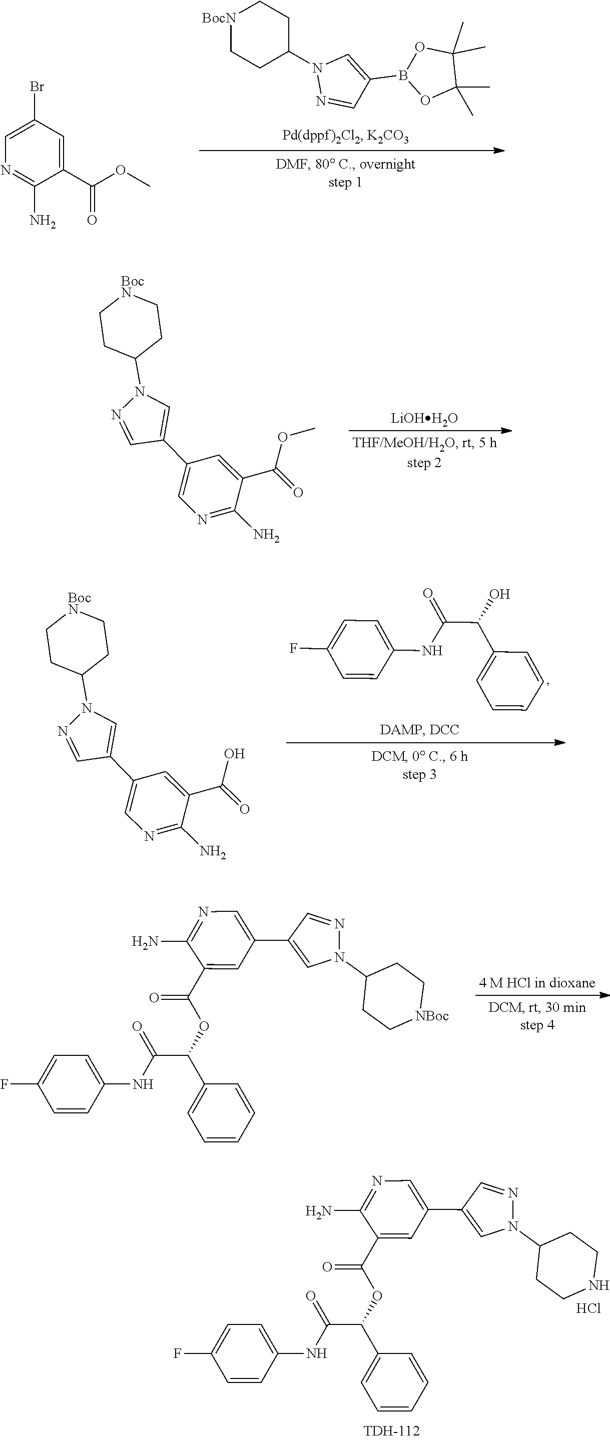

### Synthesis of methyl 2-amino-5-(1-(1-(tert-butoxycarbonyl)piperidin-4-yl)-1H-pyrazol-4-yl)nicotinate (step 1)

Methyl 2-amino-5-bromonicotinate (200 mg, 0.87 mmol, 1.0 eq.) was dissolved in DMF (17 mL). Then, Pd(dppf)Cl₂ (64 mg, 0.087 mmol, 0.1 eq.), tert-butyl 4-(4-(4,4,5,5-tetramethyl-1,3,2-dioxaborolan-2-yl)-1H-pyrazol-1-yl)piperidine-1-carboxylate Borane (490 mg, 1.3 mmol, 1.5 eq.), and potassium carbonate (478 mg, 3.5 mmol, 4.0 eq.) was added and reacted overnight at 80° C. in the presence of nitrogen. After completion of the reaction, DMF was concentrated under reduced pressure to remove, diluted with ethyl acetate, and extracted with water. The combined organic layer was washed with brine, and the residual water was removed through anhydrous Na₂SO₄, followed by filtration. After concentrating the organic layer, column chromatography was performed to obtain the desired methyl 2-amino-5-(1-(1-(tert-butoxycarbonyl)piperidin-4-yl)-1H-pyrazol-4-yl)nicotinate (291 mg, 84%).

¹H NMR (400 MHz, CDCl₃) δ 8.39-8.35 (m, 1H), 8.20 (s, 1H), 7.73 (s, 1H), 7.62 (d, J=13.4 Hz, 1H), 4.33-4.19 (m, 1H), 3.92 (s, 1H), 2.98-2.87 (m, 1H), 2.23-2.11 (m, 1H), 2.03-1.88 (m, 1H), 1.51-1.48 (m, 1H).

### Synthesis of 2-amino-5-(1-(1-(tert-butoxycarbonyl)piperidin-4-yl)-1H-pyrazol-4-yl)nicotinic acid (step 2)

Methyl 2-amino-5-(1-(1-(tert-butoxycarbonyl)piperidin-4-yl)-1H-pyrazol-4-yl)nicotinate (281 mg, 0.73 mmol, 1 eq.) was dissolved in THF/MeOH/H₂O (2 mL/1 mL/1 mL). Then, LiOH·H₂O (153 mg, 3.7 mmol, 5 eq.) was added and reacted at room temperature for 5 hours. After completion of the reaction, the reaction solvent was concentrated, diluted with EtOAc, and extracted with water. The aqueous layer was adjusted to pH 4-5 with 1 N HCl aqueous solution and then extracted with EtOAc. The combined organic layer was washed with brine, and the residual water was removed through anhydrous Na₂SO₄, followed by filtration. The organic layer was concentrated under reduced pressure to obtain the desired 2-amino-5-(1-(1-(tert-butoxycarbonyl)piperidin-4-yl)-1H-pyrazol-4-yl)nicotinic acid (259 mg, 92%).

¹H NMR (300 MHz, DMSO) δ 8.49-8.40 (m, 1H), 8.31-8.21 (m, 1H), 8.21-8.15 (m, 1H), 7.89-7.78 (m, 1H), 4.45-4.26 (m, 1H), 4.14-3.98 (m, 1H), 3.39-3.18 (m, 2H), 3.04-2.81 (m, 2H), 2.09-1.99 (m, 1H), 1.86-1.71 (m, 2H), 1.54-1.28 (m, 2H), 1.41 (d, 9H).

### Synthesis of (R)-2-((4-fluorophenyl)amino)-2-oxo-1-phenylethyl 2-amino-5-(1-(1-(tert-butoxycarbonyl)piperidin-4-yl)-1H-pyrazol-4-yl)nicotinate (step 3)

2-amino-5-(1-(1-(tert-butoxycarbonyl)piperidin-4-yl)-1H-pyrazol-4-yl)nicotinic acid (50 mg, 0.13 mmol, 1 eq.) was dissolved in DCM (1.0 mL). Then, (R)—N-(4-Fluorophenyl)-2-hydroxy-2-phenylacetamide (32 mg, 0.13 mmol, 1 eq.), DCC (27 mg, 0.13 mmol, 1 eq.), and DMAP (8 mg, 0.07 mmol, 0.5 eq.) were added and reacted at room temperature for 6 hours. After completion of the reaction, it was diluted with DCM and washed with water. The combined organic layer was washed with brine, and the residual water was removed through anhydrous Na₂SO₄, followed by filtration. After concentrating the organic layer, column chromatography was performed to obtain the desired (R)-2-((4-fluorophenyl)amino)-2-oxo-1-phenylethyl 2-amino-5-(1-(1-(tert-butoxycarbonyl)piperidin-4-yl)-1H-pyrazol-4-yl)nicotinate (71 mg, 92%).

¹H NMR (400 MHz, CDCl₃) δ 8.43-8.39 (m, 1H), 8.28-8.25 (m, 1H), 8.24-8.21 (m, 1H), 7.99-7.92 (m, 1H), 7.72-7.68 (m, 1H), 7.62-7.58 (m, 2H), 7.49-7.44 (m, 4H), 7.04-6.96 (m, 2H), 6.50-6.48 (m, 1H), 6.40-6.33 (m, 2H), 4.36-4.19 (m, 3H), 2.97-2.83 (m, 2H), 2.21-2.09 (m, 2H), 2.02-1.87 (m, 3H), 1.51 (s, 9H).

### Synthesis of (R)-2-((4-fluorophenyl)amino)-2-oxo-1-phenylethyl 2-amino-5-(1-(piperidin-4-yl)-1H-pyrazol-4-yl)nicotinate hydrochloride (TDH-112, step 4)

(R)-2-((4-fluorophenyl)amino)-2-oxo-1-phenylethyl 2-amino-5-(1-(1-(tert-butoxycarbonyl)piperidin-4-yl)-1H-pyrazol-4-yl)nicotinate (60 mg, 0.098 mmol) was dissolved in DCM (1.5 mL). Then, 4N HCl in dioxane (0.5 mL) was slowly added, and the mixture was stirred at room temperature for 30 minutes. After completion of the reaction, the precipitate was filtered to obtain the desired (R)-2-((4-fluorophenyl)amino)-2-oxo-1-phenylethyl 2-amino-5-(1-(piperidin-4-yl)-1H-pyrazol-4-yl)nicotinate hydrochloride (55 mg).

### Synthesis of TDH-180, 181, and 182

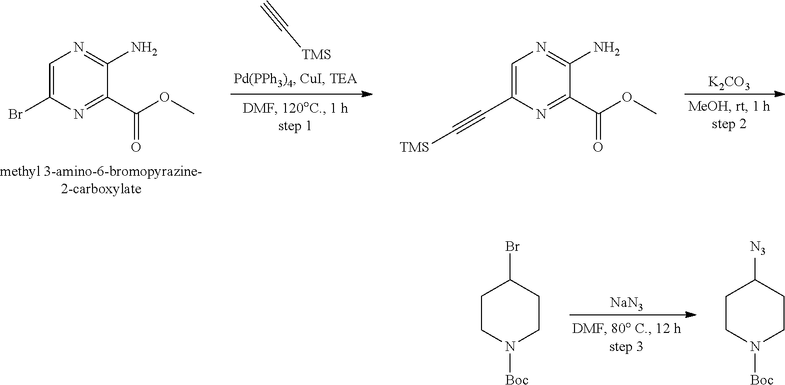

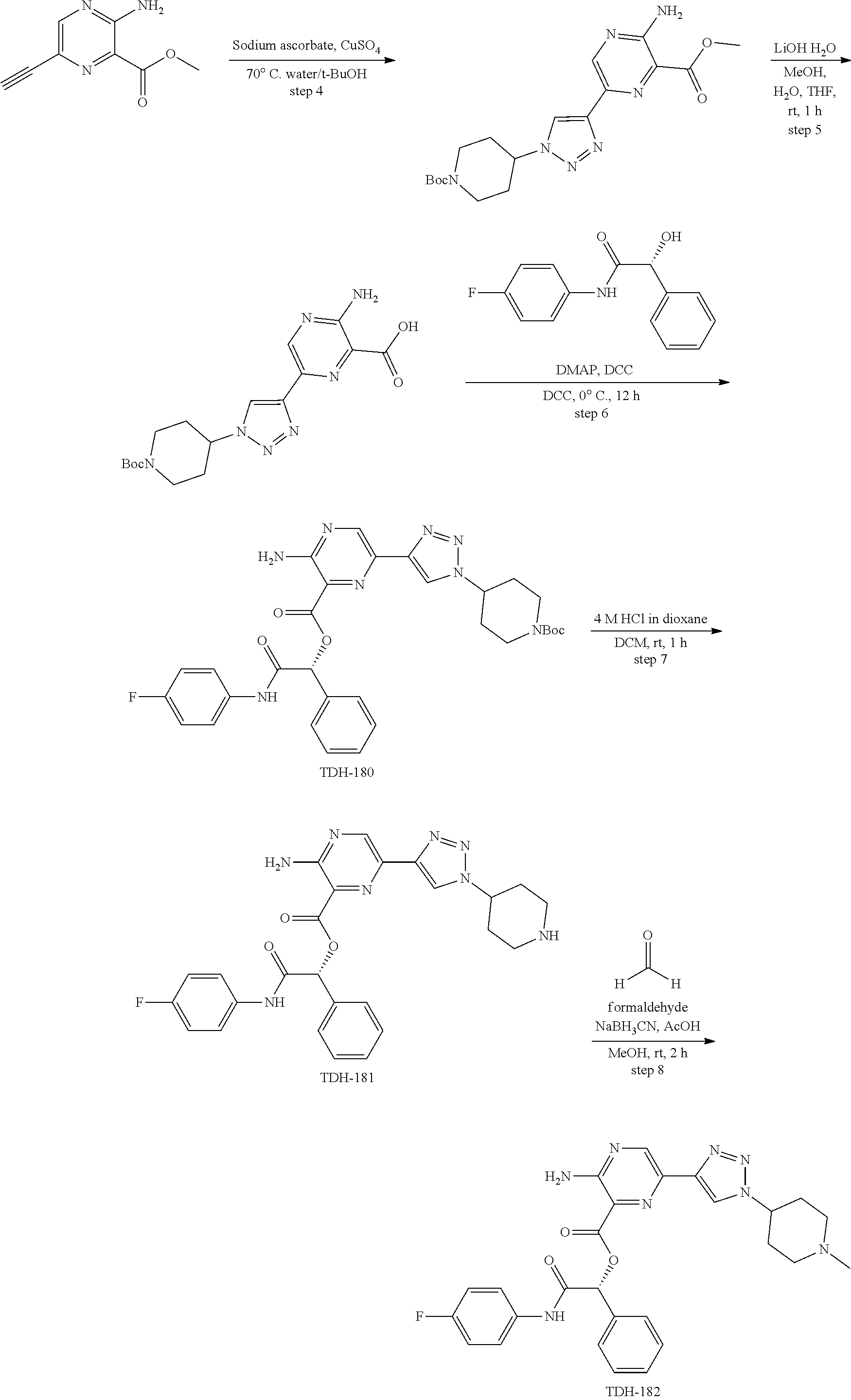

### Synthesis of methyl 3-amino-6-((trimethylsilyl)ethynyl)pyrazine-2-carboxylate (step 1)

Methyl 3-amino-6-bromopyrazine-2-carboxylate (2 g, 8.66 mmol) was dissolved in DMF (10 mL), and then, Pd(PPh₃)₄ (508 mg, 0.44 mmol), CuI (84 mg, 0.44 mmol), and TEA (4.82 mL, 34.64 mmol) were added. Ethylnyltrimethylsilane (1.80 mL, 12.98 mmol) was added thereto and reacted at 120° C. for 1 hour in the presence of nitrogen. After completion of the reaction, the mixture was diluted with water and extracted with DCM (50 mL×4). The collected organic solvent layers were washed with brine, dried over anhydrous MgSO₄, and filtered. The organic layer was concentrated under reduced pressure and then separated and purified by silica gel column chromatography to obtain the desired methyl 3-amino-6-((trimethylsilyl)ethynyl)pyrazine-2-carboxylate.

¹H NMR (300 MHz, Chloroform-d) δ 8.36 (s, 1H), 4.00 (s, 3H), 0.30-0.25 (m, 9H)

### Synthesis of methyl 3-amino-6-ethynylpyrazine-2-carboxylate (step 2)

Methyl 3-amino-6-((trimethylsilyl)ethynyl)pyrazine-2-carboxylate (1.73 g, 6.94 mmol) was dissolved in MeOH (10 mL). Then, K₂CO₃ (719 mg, 5.20 mmol) was added, and reacted at room temperature for 1 hour. After completion of the reaction, the solvent was concentrated, and the remaining K₂CO₃ was filtered and washed with MeOH. After concentration, the filtrate was separated and purified by silica gel column chromatography to obtain the desired methyl 3-amino-6-ethynylpyrazine-2-carboxylate.

¹H NMR (500 MHz, Chloroform-d) δ 8.37 (s, 1H), 4.00 (s, 3H), 3.19 (s, 1H).

### Synthesis of tert-butyl 4-azidopiperidine-1-carboxylate (step 3)

After dissolving tert-butyl 4-bromopiperidine-1-carboxylate (1 g, 3.79 mmol) in DMF (1 mL), and adding sodium azide (984 mg, 15.14 mmol), the mixture was reacted at 80° C. for 12 hours. After completion of the reaction, the mixture was diluted with water, extracted with EtOAc, and washed with brine. The collected organic layer was dried over anhydrous MgSO₄ and filtered. After concentrating the organic layer, it was separated and purified by silica gel column chromatography (EtOAc/Hexane=10%) to obtain the target tert-butyl 4-azidopiperidine-1-carboxylate.

¹H NMR (300 MHz, Chloroform-d) δ 3.90-3.74 (m, 2H), 3.62-3.50 (m, 1H), 3.15-3.02 (m, 2H), 1.95-1.78 (m, 2H), 1.61-1.46 (m, 2H), 1.45-1.35 (m, 9H).

### Synthesis of methyl 3-amino-6-(1-(1-(tert-butoxycarbonyl)piperidin-4-yl)-1H-1,2,3-triazol-4-yl)pyrazine-2-carboxylate (step 4)

Methyl 3-amino-6-ethynylpyrazine-2-carboxylate (819 mg, 4.62 mmol) and tert-butyl 4-azidopiperidine-1-carboxylate (1.25 g, 5.55 mmol) were dissolved in a mixture of water and 1-butanol, and sodium ascorbate. (366 mg, 1.85 mmol) and CuSO₄ (146 mg, 0.92 mmol) were added. The mixture was stirred for 12 hours. After completion of the reaction, the mixture was diluted with EtOAc and washed with water. The combined organic layer was filtered after drying the residue with anhydrous MgSO₄. After concentrating the organic layer, it was separated and purified by silica gel column chromatography (EtOAc/Hexane=70%) to obtain the desired methyl 3-amino-6-(1-(1-(tert-butoxycarbonyl)piperidin-4-yl)-1H-1,2,3-triazol-4-yl)pyrazine-2-carboxylate.

¹H NMR (500 MHz, Chloroform-d) δ 9.12-9.09 (m, 1H), 8.11 (d, J=1.6 Hz, 1H), 4.74-4.66 (m, 1H), 4.31 (s, 2H), 4.03-4.01 (s, 3H), 2.98 (s, 2H), 2.29-2.21 (m, 2H), 2.04-1.97 (m, 2H), 1.28 (m, 9H).

### Synthesis of 3-amino-6-(1-(1-(tert-butoxycarbonyl)piperidin-4-yl)-1H-1,2,3-triazol-4-yl)pyrazine-2-carboxylic acid (step 5)

Methyl 3-amino-6-(1-(1-(tert-butoxycarbonyl)piperidin-4-yl)-1H-1,2,3-triazol-4-yl)pyrazine-2-carboxylate (1.67 g, 4.14 mmol) was dissolved in a mixed solvent of methanol (5 mL), H₂O (5 mL), and THF (15 mL). Then, lithium hydroxide monohydrate (521 mg, 12.4 mmol) was added and reacted at room temperature for 1 hour. After concentrating the reaction solution, it was dissolved in water and the pH was adjusted to 3 using 1 N HCl. After extraction with EtOAc, the residue was dried over anhydrous MgSO₄ and filtered. The organic layer was concentrated to obtain the desired 3-amino-6-(1-(1-(tert-butoxycarbonyl)piperidin-4-yl)-1H-1,2,3-triazol-4-yl)pyrazine-2-carboxylic acid.

¹H NMR (300 MHz, Chloroform-d) δ 9.16 (s, 1H), 8.06 (s, 1H), 4.80-4.45 (m, 1H), 4.33 (s, 2H), 3.30-2.88 (m, 2H), 2.32-2.25 (m, 2H), 2.10-1.85 (m, 3H), 1.51 (s, 9H).

### Synthesis of (R)-2-((4-fluorophenyl)amino)-2-oxo-1-phenylethyl 3-amino-6-(1-(1-(tert-butoxycarbonyl)piperidin-4-yl)-1H-1,2,3-triazol-4-yl)pyrazine-2-carboxylate (TD-180, step 6)

3-amino-6-(1-(1-(tert-butoxycarbonyl)piperidin-4-yl)-1H-1,2,3-triazol-4-yl)pyrazine-2-carboxylic acid (30 mg, 0.08 mmol) was dissolved in DCM (2 mL). Then, ARG-19-146 (20 mg, 0.08 mmol), DMAP (5 mg, 0.04 mmol), and DCC (16 mg, 0.08 mmol) were added thereto, and then reacted at room temperature for 12 hours. After completion of the reaction, extraction was performed with DCM and H₂O, and the organic layer was dried with anhydrous MgSO₄ and the residue was filtered. After concentrating the organic layer, it was separated and purified by silica gel column chromatography (EtOAc/Hexane=50%) to obtain the desired (R)-2-((4-fluorophenyl)amino)-2-oxo-1-phenylethyl 3-amino-6-(1-(1-(tert-butoxycarbonyl)piperidin-4-yl)-1H-1,2,3-triazol-4-yl)pyrazine-2-carboxylate.

### Synthesis of (R)-2-((4-fluorophenyl)amino)-2-oxo-1-phenylethyl 3-amino-6-(1-(piperidin-4-yl)-1H-1,2,3-triazol-4-yl)pyrazine-2-carboxylate (TD-181, step 7)

(R)-2-((4-fluorophenyl)amino)-2-oxo-1-phenylethyl 3-amino-6-(1-(1-(tert-butoxycarbonyl)piperidin-4-yl)-1H-1,2,3-triazol-4-yl)pyrazine-2-carboxylate (361 mg, 0.59 mmol) was dissolved in DCM (1 mL), 4M HCl in dioxane (2.0 mL) was added, and the mixture was stirred at room temperature for 1 hour. The reaction solvent was concentrated, dissolved in water, neutralized using a saturated NaHCO₃ aqueous solution, extracted with EA, and concentrated to obtain the desired (R)-2-((4-fluorophenyl)amino)-2-oxo-1-phenylethyl 3-amino-6-(1-(piperidin-4-yl)-1H-1,2,3-triazol-4-yl)pyrazine-2-carboxylate.

### Synthesis of (R)-2-((4-fluorophenyl)amino)-2-oxo-1-phenylethyl 3-amino-6-(1-(1-methylpiperidin-4-yl)-1H-1,2,3-triazol-4-yl)pyrazine-2-carboxylate (TD-182, step 8)

(R)-2-((4-fluorophenyl)amino)-2-oxo-1-phenylethyl 3-amino-6-(1-(piperidin-4-yl)-1H-1,2,3-triazol-4-yl)pyrazine-2-carboxylate (15 mg, 0.03 mmol) was dissolved in MeOH (0.40 mL). Then, formaldehyde (0.001 mL, 0.05 mmol) and AcOH (0.10 mL) were added thereto and stirred for 1 hour. NaBH₃CN (11 mg, 0.18 mmol) was added thereto and reacted at room temperature for 2 hours. After the reaction was terminated by adding 5% K₂CO₃, extraction was performed with EtOAc, and the collected organic layers were dried over anhydrous MgSO₄ and filtered. After concentrating the organic layer, it was separated and purified by silica gel column chromatography (DCM/MeOH=6%) to obtain the desired (R)-2-((4-fluorophenyl)amino)-2-oxo-1-phenylethyl 3-amino-6-(1-(1-methylpiperidin-4-yl)-1H-1,2,3-triazol-4-yl)pyrazine-2-carboxylate.

### Synthesis of TDH-183

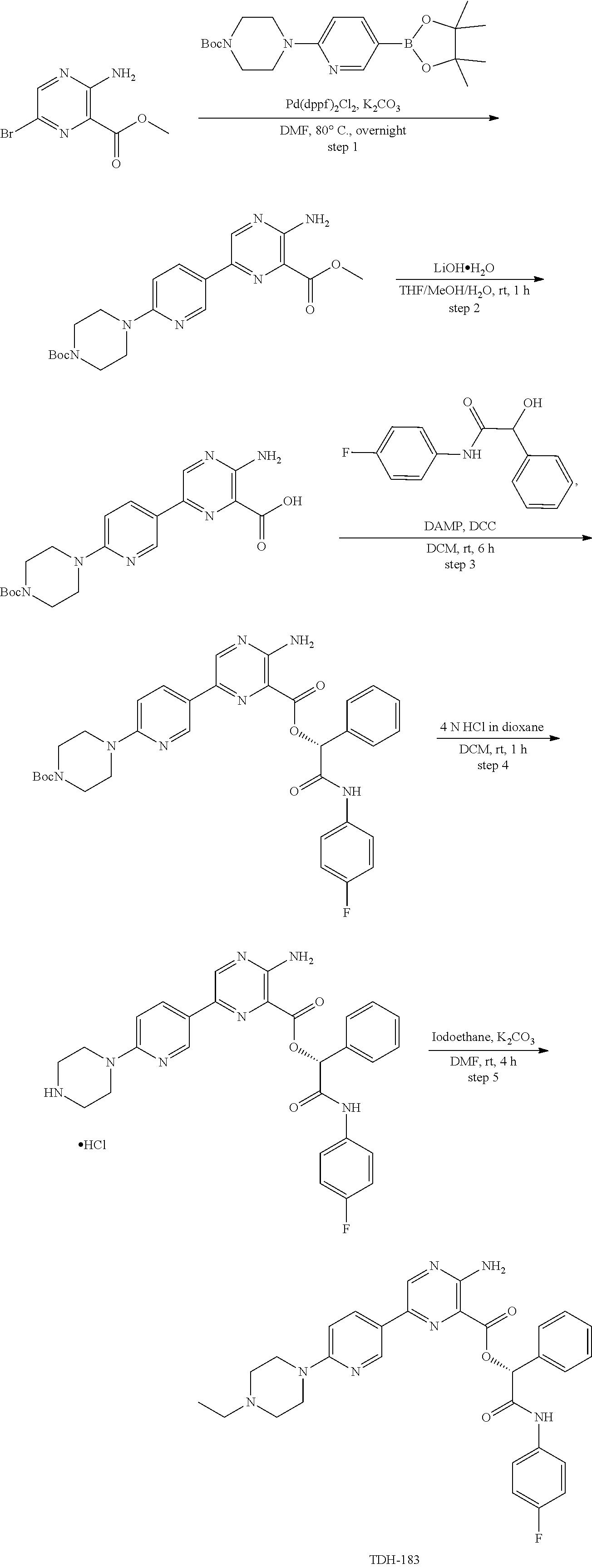

### Synthesis of methyl 3-amino-6-(6-(4-(tert-butoxycarbonyl)piperazin-1-yl)pyridin-3-yl)pyrazine-2-carboxylate (step 1)

Methyl 3-amino-6-bromopyrazine-2-carboxylate (40 mg, 0.17 mmol, 1 eq.) was dissolved in DMF (1 mL). Then, Pd(dppf)Cl₂ (13 mg, 0.017 mmol, 0.1 eq.), tert-butyl 4-(5-(4,4,5,5-tetramethyl-1,3,2-dioxaborolan-2-yl)pyridin-2-yl)piperazine-1-carboxylate (101 mg, 0.26 mmol, 1.5 eq.), and potassium carbonate (95 mg, 0.69 mmol, 4.0 eq.) were added and reacted overnight at 80° C. in the presence of nitrogen. After completion of the reaction, DMF was concentrated under reduced pressure to remove. The reactant was diluted with ethyl acetate, and extracted with water. The combined organic layer was washed with brine, and the residual water was removed through anhydrous Na₂SO₄, followed by filtration. After concentrating the organic layer, column chromatography was performed to obtain the desired Methyl 3-amino-6-(6-(4-(tert-butoxycarbonyl)piperazin-1-yl)pyridin-3-yl)pyrazine-2-carboxylate (94 mg).

¹H NMR (300 MHz, CDCl₃) δ 8.79-8.70 (m, 1H), 8.62 (s, 1H), 8.17-8.05 (m, 1H), 6.78-6.71 (m, 1H), 6.43 (s, 2H), 4.02 (s, 3H), 3.66-3.58 (m, 8H), 1.51 (s, 9H).

### Synthesis of 3-amino-6-(6-(4-(tert-butoxycarbonyl)piperazin-1-yl)pyridin-3-yl)pyrazine-2-carboxylic acid (step 2)

Methyl 3-amino-6-(6-(4-(tert-butoxycarbonyl)piperazin-1-yl)pyridin-3-yl)pyrazine-2-carboxylate (94 mg, 0.23 mmol, 1 eq.) was dissolved in THF/MeOH/H₂O (1.0 mL/0.2 mL/0.2 mL). Then, LiOH·H₂O (48 mg, 1.1 mmol, 5.0 eq.) was added and reacted at room temperature for 1 hour. After completion of the reaction, the reaction solvent was concentrated, diluted with EtOAc, and extracted with water. The aqueous layer was adjusted to pH 4-5 with 1 N HCl aqueous solution and then extracted with EtOAc. The combined organic layer was washed with brine, and the residual water was removed through anhydrous Na₂SO₄, followed by filtration. The organic layer was concentrated under reduced pressure to obtain the desired 3-amino-6-(6-(4-(tert-butoxycarbonyl)piperazin-1-yl)pyridin-3-yl)pyrazine-2-carboxylic acid (45 mg, 49%).

¹H NMR (300 MHz, MeOD) δ 8.75 (s, 1H), 8.65 (s, 1H), 8.31-8.20 (m, 1H), 6.95 (d, J=9.0 Hz, 1H), 3.71-3.50 (m, 8H), 1.50 (s, 9H).

### Synthesis of (R)-2-((4-fluorophenyl)amino)-2-oxo-1-phenylethyl 3-amino-6-(6-(4-(tert-butoxycarbonyl)piperazin-1-yl)pyridin-3-yl)pyrazine-2-carboxylate (step 3)

3-amino-6-(6-(4-(tert-butoxycarbonyl)piperazin-1-yl)pyridin-3-yl)pyrazine-2-carboxylic acid (45 mg, 0.11 mmol, 1 eq.) was dissolved in DCM (1 mL). Then, after adding ARG (28 mg, 0.11 mmol, 1 eq.), DCC (23 mg, 0.11 mmol, 1 eq.), and DMAP (7 mg, 0.06 mmol, 0.5 eq.) thereto, the mixture was reacted at room temperature for 6 hours. After completion of the reaction, it was diluted with DCM and washed with water. The combined organic layer was washed with brine, and the residual water was removed through anhydrous Na₂SO₄, followed by filtration. After concentrating the organic layer, column chromatography was performed to obtain the desired (R)-2-((4-fluorophenyl)amino)-2-oxo-1-phenylethyl 3-amino-6-(6-(4-(tert-butoxycarbonyl)piperazin-1-yl)pyridin-3-yl)pyrazine-2-carboxylate (46 mg, 68%).

¹H NMR (300 MHz, CDCl₃) δ 8.99 (s, 1H), 8.85 (s, 1H), 8.70 (s, 1H), 8.16-8.05 (m, 1H), 7.70-7.62 (m, 2H), 7.63-7.55 (m, 2H), 7.47-7.39 (m, 3H), 7.06-6.96 (m, 2H), 6.83-6.72 (m, 1H), 6.41 (d, J=23.0 Hz, 3H), 3.69-3.58 (m, 8H), 1.52 (s, 9H).

### Synthesis of (R)-2-((4-fluorophenyl)amino)-2-oxo-1-phenylethyl 3-amino-6-(6-(piperazin-1-yl)pyridin-3-yl)pyrazine-2-carboxylate hydrochloride (step 4)

(R)-2-((4-fluorophenyl)amino)-2-oxo-1-phenylethyl 3-amino-6-(6-(4-(tert-butoxycarbonyl)piperazin-1-yl)pyridin-3-yl)pyrazine-2-carboxylate (46 mg, 0.07 mmol) was dissolved in DCM (1.5 mL). Then, 4N HCl in dioxane (1 mL) was slowly added, and the mixture was stirred at room temperature for 1 hour. After completion of the reaction, the precipitate was filtered to obtain the desired (R)-2-((4-fluorophenyl)amino)-2-oxo-1-phenylethyl 3-amino-6-(6-(piperazin-1-yl)pyridin-3-yl)pyrazine-2-carboxylate hydrochloride (47 mg).

¹H NMR (300 MHz, MeOD) δ 8.87 (s, 1H), 8.82-8.75 (m, 1H), 8.68-8.64 (m, 1H), 7.80-7.72 (m, 2H), 7.65-7.55 (m, 3H), 7.55-7.44 (m, 3H), 7.13-7.02 (m, 2H), 6.33 (s, 1H), 4.13-4.05 (m, 4H), 3.55-3.50 (m, 4H).

### Synthesis of (R)-2-((4-fluorophenyl)amino)-2-oxo-1-phenylethyl 3-amino-6-(6-(4-ethylpiperazin-1-yl)pyridin-3-yl)pyrazine-2-carboxylate (TDH-183, step 5)

(R)-2-((4-fluorophenyl)amino)-2-oxo-1-phenylethyl 3-amino-6-(6-(piperazin-1-yl)pyridin-3-yl)pyrazine-2-carboxylate hydrochloride (8 mg, 0.015 mmol, 1 eq.) was dissolved in DMF (1.0 mL), and then K₂CO₃ (4 mg, 0.03 mmol, 2.0 eq.) was added. After adding iodoethane (0.002 mL, 0.0023 mmol, 1.5 eq.) to the above solution, it was reacted at room temperature for 4 hours. After completion of the reaction, the mixture was diluted with ethyl acetate and extracted with water. The combined organic layer was washed with brine, and the residual water was removed through anhydrous Na₂SO₄, followed by filtration. After concentrating the organic layer, column chromatography was performed to obtain the desired (R)-2-((4-fluorophenyl)amino)-2-oxo-1-phenylethyl 3-amino-6-(6-(4-ethylpiperazin-1-yl)pyridin-3-yl)pyrazine-2-carboxylate (5 mg, 60%).

### Synthesis of TDH-184

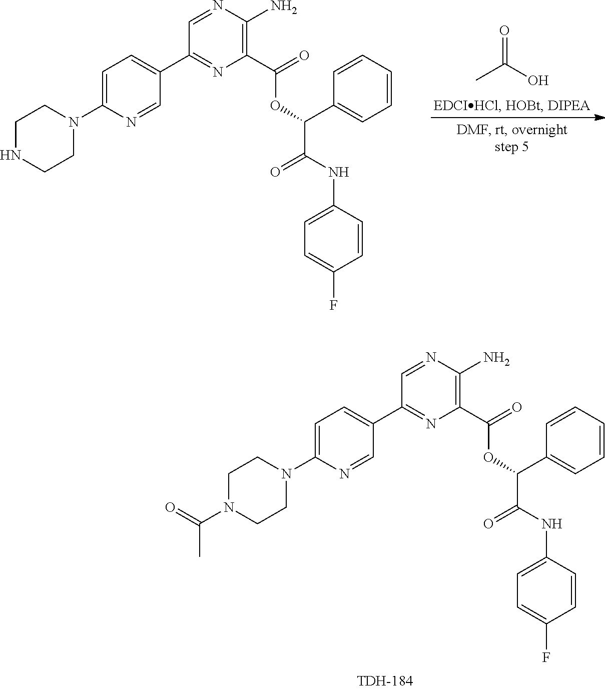

(R)-2-((4-fluorophenyl)amino)-2-oxo-1-phenylethyl 3-amino-6-(6-(piperazin-1-yl)pyridin-3-yl)pyrazine-2-carboxylate hydrochloride (8 mg, 0.015 mmol, 1 eq.) was dissolved in DMF (1 mL). Then, acetic acid (0.001 mL, 0.015 mmol, 1.0 eq.), EDCI-HCl (3 mg, 0.017 mmol, 1.1 eq.), HOBt (3 mg, 0.017 mmol, 1.1 eq.), and DIPEA (0.0013 mL, 0.076 mmol, 5 eq.) were added, and then reacted overnight at room temperature. After completion of the reaction, the mixture was diluted with ethyl acetate and extracted with water. The combined organic layer was washed with brine, and the residual water was removed through anhydrous Na₂SO₄, followed by filtration. After concentrating the organic layer, column chromatography was performed to obtain the desired (R)-2-((4-fluorophenyl)amino)-2-oxo-1-phenylethyl 6-(6-(4-acetylpiperazin-1-yl)pyridin-3-yl)-3-aminopyrazine-2-carboxylate (6 mg, 59%).

### Synthesis of TDH-185

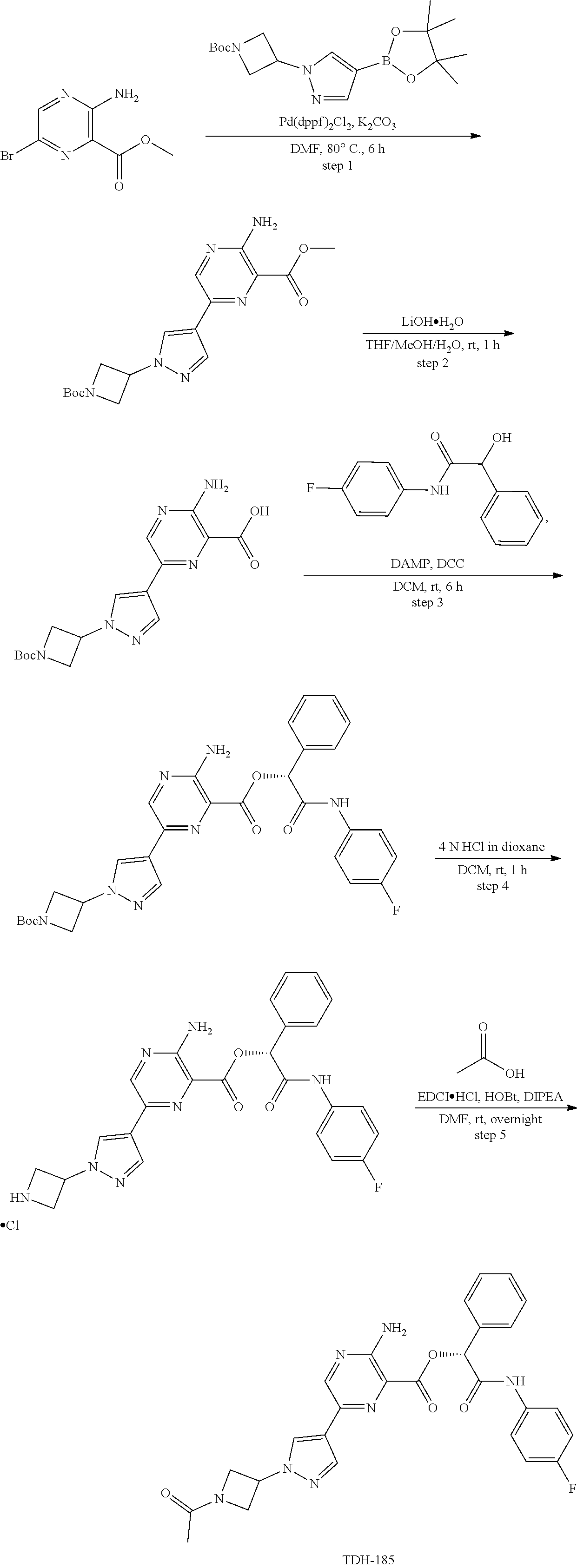

### Synthesis of methyl 3-amino-6-(1-(1-(tert-butoxycarbonyl)azetidin-3-yl)-1H-pyrazol-4-yl)pyrazine-2-carboxylate (step 1)

Methyl 3-amino-6-bromopyrazine-2-carboxylate (40 mg, 0.17 mmol, 1 eq.) was dissolved in DMF (1 mL). Then, Pd(dppf)Cl₂ (13 mg, 0.017 mmol, 0.1 eq.), tert-butyl 3-(4-(4,4,5,5-tetramethyl-1,3,2-dioxaborolan-2-yl)-1H-pyrazol-1-yl)azetidine-1-carboxylate (90 mg, 0.26 mmol, 1.5 eq.), and potassium carbonate (95 mg, 0.69 mmol, 4.0 eq.) were added and reacted at 80° C. for 6 hours in the presence of nitrogen. After completion of the reaction, DMF was concentrated under reduced pressure to remove, diluted with ethyl acetate, and extracted with water. The combined organic layer was washed with brine, and the residual water was removed through anhydrous Na₂SO₄, followed by filtration. After concentrating the organic layer, column chromatography was performed to obtain the desired methyl 3-amino-6-(1-(1-(tert-butoxycarbonyl)azetidin-3-yl)-1H-pyrazol-4-yl)pyrazine-2-carboxylate (50 mg, 79%).

¹H NMR (400 MHz, CDCl₃) δ 8.47 (s, 1H), 8.07 (s, 1H), 7.99 (s, 1H), 6.42 (s, 2H), 5.16-5.05 (m, 1H), 4.47-4.37 (m, 4H), 4.02 (s, 3H), 1.49 (s, 9H).

### Synthesis of 3-amino-6-(1-(1-(tert-butoxycarbonyl)azetidin-3-yl)-1H-pyrazol-4-yl)pyrazine-2-carboxylic acid (step 2)

Methyl 3-amino-6-(1-(1-(tert-butoxycarbonyl)azetidin-3-yl)-1H-pyrazol-4-yl)pyrazine-2-carboxylate (50 mg, 0.13 mmol, 1 eq.) was dissolved in THF/MeOH/H₂O (0.5 mL/0.1 mL/0.1 mL). Then, LiOH·H₂O (28 mg, 0.67 mmol, 5.0 eq.) was added and reacted at room temperature for 1 hour. After completion of the reaction, the reaction solvent was concentrated, diluted with EtOAc, and extracted with water. The aqueous layer was adjusted to pH 4-5 with 1N HCl aqueous solution and then extracted with EtOAc. The combined organic layer was washed with brine, and the residual water was removed through anhydrous Na₂SO₄, followed by filtration. The organic layer was concentrated under reduced pressure to obtain the desired 3-amino-6-(1-(1-(tert-butoxycarbonyl)azetidin-3-yl)-1H-pyrazol-4-yl)pyrazine-2-carboxylic acid (38 mg, 83%).

¹H NMR (300 MHz, MeOD) δ 8.58 (s, 1H), 8.40 (s, 1H), 8.19 (s, 1H), 5.32-5.19 (m, 1H), 4.50-4.38 (m, 2H), 4.38-4.28 (m, 2H), 1.50 (s, 9H).

### Synthesis of (R)-2-((4-fluorophenyl)amino)-2-oxo-1-phenylethyl 3-amino-6-(1-(1-(tert-butoxycarbonyl)azetidin-3-yl)-1H-pyrazol-4-yl)pyrazine-2-carboxylate (step 3)

3-amino-6-(1-(1-(tert-butoxycarbonyl)azetidin-3-yl)-1H-pyrazol-4-yl)pyrazine-2-carboxylic acid (38 mg, 0.11 mmol, 1 eq.) was dissolved in DCM (1 mL). Then, (R)—N-(4-Fluorophenyl)-2-hydroxy-2-phenylacetamide (26 mg, 0.11 mmol, 1 eq.), DCC (22 mg, 0.11 mmol, 1 eq.), and DMAP (7 mg, 0.05 mmol, 0.5 eq.) were added and reacted at room temperature for 6 hours. After completion of the reaction, it was diluted with DCM and washed with water. The combined organic layer was washed with brine, and the residual water was removed through anhydrous Na₂SO₄, followed by filtration. After concentrating the organic layer, column chromatography was performed to obtain the desired (R)-2-((4-fluorophenyl)amino)-2-oxo-1-phenylethyl 3-amino-6-(1-(1-(tert-butoxycarbonyl)azetidin-3-yl)-1H-pyrazol-4-yl)pyrazine-2-carboxylate (41 mg, 65%).

¹H NMR (300 MHz, CDCl₃) δ 8.88 (s, 1H), 8.54 (s, 1H), 8.11-8.06 (m, 1H), 8.04-7.97 (m, 1H), 7.70-7.62 (m, 2H), 7.58-7.50 (m, 2H), 7.47-7.37 (m, 3H), 7.09-6.99 (m, 2H), 6.51-6.30 (m, 3H), 5.19-5.05 (m, 1H), 4.48-4.34 (m, 4H), 1.49 (s, 9H).

### Synthesis of (R)-2-((4-fluorophenyl)amino)-2-oxo-1-phenylethyl 3-amino-6-(1-(azetidin-3-yl)-1H-pyrazol-4-yl)pyrazine-2-carboxylate hydrochloride (step 4)

(R)-2-((4-fluorophenyl)amino)-2-oxo-1-phenylethyl 3-amino-6-(1-(1-(tert-butoxycarbonyl)azetidin-3-yl)-1H-pyrazol-4-yl)pyrazine-2-carboxylate (41 mg, 0.07 mmol) was dissolved in DCM (1.5 mL). 4N HCl in dioxane (0.8 mL) was slowly added, and the mixture was stirred at room temperature for 1 hour. After completion of the reaction, the precipitate was filtered to obtain the desired (R)-2-((4-fluorophenyl)amino)-2-oxo-1-phenylethyl 3-amino-6-(1-(azetidin-3-yl)-1H-pyrazol-4-yl)pyrazine-2-carboxylate hydrochloride (42 mg).

¹H NMR (400 MHz, MeOD) δ 8.60 (s, 1H), 8.28 (s, 1H), 8.08 (s, 1H), 7.77-7.71 (m, 2H), 7.63-7.55 (m, 2H ¹H NMR (300 MHz, MeOD) δ 8.63-8.51 (m, 1H), 8.25-8.14 (m, 2H), 7.74-7.67 (m, 1H), 7.61-7.50 (m, 3H), 7.47-7.38 (m, 2H), 7.37-7.28 (m, 1H), 7.08-6.96 (m, 2H), 6.31 (s, 1H), 5.51-5.43 (m, 1H), 4.61-4.51 (m, 4H).

### Synthesis of (R)-2-((4-fluorophenyl)amino)-2-oxo-1-phenylethyl 6-(1-(1-acetylazetidin-3-yl)-1H-pyrazol-4-yl)-3-aminopyrazine-2-carboxylate (TDH-185, step 5)

(R)-2-((4-fluorophenyl)amino)-2-oxo-1-phenylethyl 3-amino-6-(1-(azetidin-3-yl)-1H-pyrazol-4-yl)pyrazine-2-carboxylate hydrochloride (6 mg, 0.012 mmol, 1 eq.) was dissolved in DMF (1.0 mL). Then, acetic acid (0.001 mL, 0.012 mmol, 1.0 eq.), EDCI-HCl (3 mg, 0.014 mmol, 1.1 eq.), HOBt (2 mg, 0.014 mmol, 1.1 eq.), and DIPEA (0.0011 mL, 0.06 mmol, 5 eq.) were added and reacted at room temperature overnight. After completion of the reaction, the mixture was diluted with ethyl acetate and extracted with water. The combined organic layer was washed with brine, and the residual water was removed through anhydrous Na₂SO₄, followed by filtration. After concentrating the organic layer, column chromatography was performed to obtain the desired (R)-2-((4-fluorophenyl)amino)-2-oxo-1-phenylethyl 6-(1-(1-acetylazetidin-3-yl)-1H-pyrazol-4-yl)-3-aminopyrazine-2-carboxylate (2 mg, 31%).

### Synthesis of TDH-227 and TDH-228

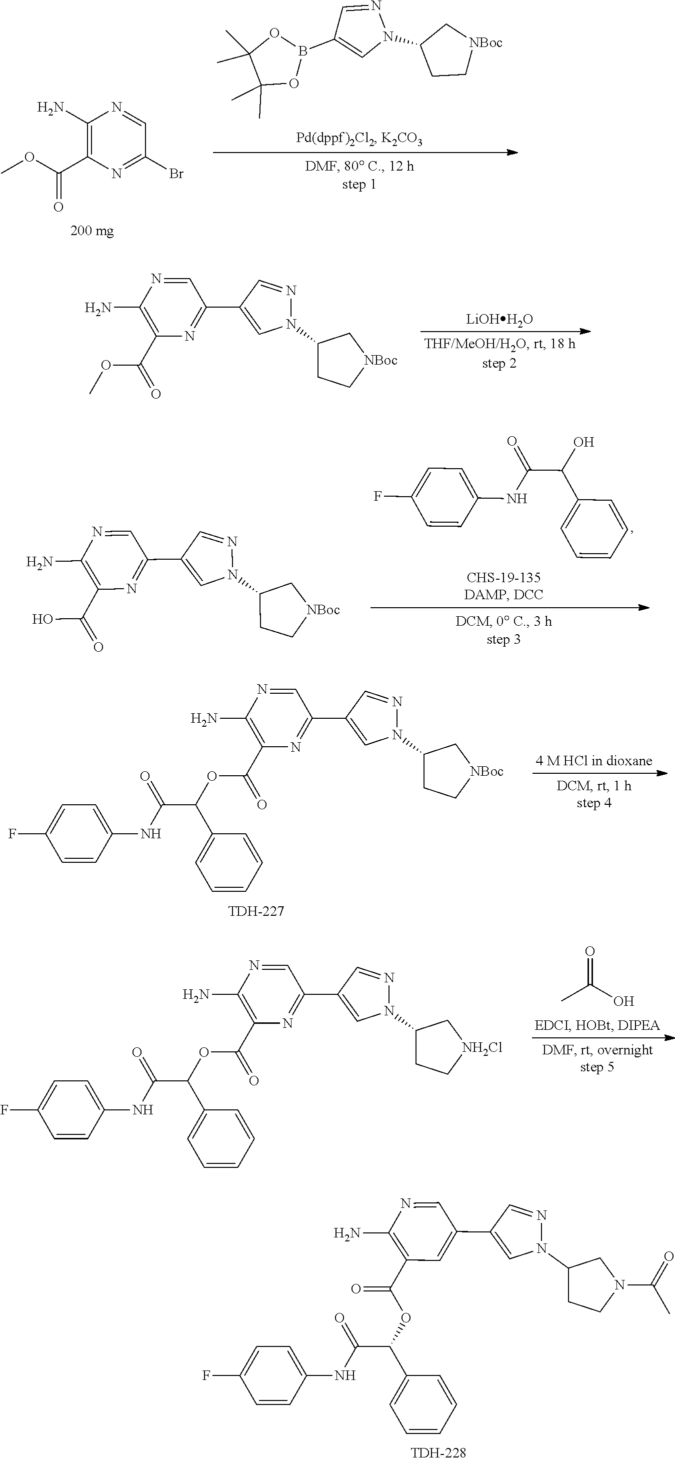

### Synthesis of methyl (S)-3-amino-6-(1-(1-(tert-butoxycarbonyl)pyrrolidin-3-yl)-1H-pyrazol-4-yl)pyrazine-2-carboxylate (step 1)

Methyl 3-amino-6-bromopyrazine-2-carboxylate (200 mg, 0.861 mmol), tert-butyl (S)-3-(4-(4,4,5,5-tetramethyl-1,3,2-dioxaborolan-2-yl)-1H-pyrazol-1-yl)pyrrolidine-1-carboxylate (469 mg, 1.29 mmol), and Potassium carbonate (475 mg, 3.44 mmol) were dissolved in DMF (7 mL), and nitrogen gas was charged. Then, Pd(dppf)Cl₂ (63 mg, 0.086 mmol) was added and reacted overnight at 80° C. After completion of the reaction, it was diluted with water, extracted with EtOAc, dried with MgSO₄, and separated and purified by silica gel column chromatography (EtOAc/Hex=80%) to obtain methyl (S)-3-amino-6-(1-(1-(tert-butoxycarbonyl)pyrrolidin-3-yl)-1H-pyrazol-4-yl)pyrazine-2-carboxylate (328 mg, 0.844 mmol, 98%) as a yellow solid.

¹H NMR (300 MHz, CDCl₃) δ 8.47 (s, 1H), 7.30 (s, 1H), 6.43 (s, 2H), 5.02-4.87 (m, 1H), 4.02 (s, 3H), 3.97-3.73 (m, 2H), 3.73-3.49 (m, 2H), 2.56-2.37 (m, 2H), 1.51 (s, 9H).

### Synthesis of (S)-3-amino-6-(1-(1-(tert-butoxycarbonyl)pyrrolidin-3-yl)-1H-pyrazol-4-yl)pyrazine-2-carboxylic acid (step 2)

Methyl (S)-3-amino-6-(1-(1-(tert-butoxycarbonyl)pyrrolidin-3-yl)-1H-pyrazol-4-yl)pyrazine-2-carboxylate (328 mg, 0.844 mmol) was dissolved in MeOH (2 mL), THF (6 mL), and water (2 mL). Then, and LiOH H₂O (88 mg, 2.11 mmol) was added and reacted overnight at room temperature. After completion of the reaction, the organic solvent was concentrated, adjusted to pH 4 using 1 N HCl, and then extracted with EtOAc (50 mL×2). The organic solvent was dried and concentrated with anhydrous MgSO₄ to obtain (S)-3-amino-6-(1-(1-(tert-butoxycarbonyl)pyrrolidin-3-yl)-1H-pyrazol-4-yl)pyrazine-2-carboxylic acid (304 mg, 0.811 mmol, 96%) as a yellow solid.

¹H NMR (300 MHz, DMSO-d₆) δ 8.58 (s, 1H), 8.37 (s, 1H), 8.06 (s, 1H), 7.38 (s, 2H), 5.05-4.89 (m, 1H), 3.82-3.68 (m, 2H), 3.63-3.52 (m, 2H), 2.42-2.25 (m, 3H), 1.40 (s, 9H).

### Synthesis of 2-((4-fluorophenyl)amino)-2-oxo-1-phenylethyl 3-amino-6-(1-((S)-1-(tert-butoxycarbonyl)pyrrolidin-3-yl)-1H-pyrazol-4-yl)pyrazine-2-carboxylate (TDH-227, step 3)

(S)-3-amino-6-(1-(1-(tert-butoxycarbonyl)pyrrolidin-3-yl)-1H-pyrazol-4-yl)pyrazine-2-carboxylic acid (304 mg, 0.812 mmol) was dissolved in DCM. Then, CHS-19-135 (199 mg, 0.812 mmol), DCC (167 mg, 0.812 mmol), and DMAP (49 mg, 0.405 mmol) were added at 0° C. and reacted overnight at room temperature. After completion of the reaction, the mixture was diluted with DCM, washed with water, dried over anhydrous MgSO₄, filtered and concentrated. The concentrate was purified by silica gel column chromatography (EtOAc/Hex=65%) to obtain a yellow solid, 2-((4-fluorophenyl)amino)-2-oxo-1-phenylethyl 3-amino-6-(1-((S)-1-(tert-butoxycarbonyl)pyrrolidin-3-yl)-1H-pyrazol-4-yl)pyrazine-2-carboxylate (319 mg, 0.530 mmol, 62%).

### Synthesis of 2-((4-fluorophenyl)amino)-2-oxo-1-phenylethyl 3-amino-6-(1-((S)-pyrrolidin-3-yl)-1H-pyrazol-4-yl)pyrazine-2-carboxylate (step 4)

2-((4-fluorophenyl)amino)-2-oxo-1-phenylethyl 3-amino-6-(1-((S)-1-(tert-butoxycarbonyl)pyrrolidin-3-yl)-1H-pyrazol-4-yl)pyrazine-2-carboxylate (319 mg, 0.530 mmol) was dissolved in DCM (6 mL). Then, 4 M HCl in dioxane (2 mL) was slowly added at 0° C., and the mixture was reacted at room temperature for 30 minutes. After completion of the reaction, it was concentrated and washed with ether to obtain 2-((4-fluorophenyl)amino)-2-oxo-1-phenylethyl 3-amino-6-(1-((S)-pyrrolidin-3-yl)-1H-pyrazol-4-yl)pyrazine-2-carboxylate (173 mg, 0.321 mmol, 60%) as a yellow solid.

¹H NMR (300 MHz, DMSO-d₆) δ 10.76 (s, 1H), 9.38 (s, 2H), 8.72 (s, 1H), 8.37 (s, 1H), 8.05 (s, 1H), 7.80-7.72 (m, 2H), 7.69-7.58 (m, 2H), 7.55-7.40 (m, 3H), 7.25-7.10 (m, 2H), 6.27 (s, 1H), 5.33-5.17 (m, 1H), 3.72-3.63 (m, 2H).

### Synthesis of 2-((4-fluorophenyl)amino)-2-oxo-1-phenylethyl 6-(1-((S)-1-acetylpyrrolidin-3-yl)-1H-pyrazol-4-yl)-3-aminopyrazine-2-carboxylate (TDH-228, step 5)

2-((4-fluorophenyl)amino)-2-oxo-1-phenylethyl 3-amino-6-(1-((S)-pyrrolidin-3-yl)-1H-pyrazol-4-yl)pyrazine-2-carboxylate (12 mg, 0.023 mmol) was dissolved in DMF. Next, acetic acid (0.002 mL, 0.023 mmol), EDCI HCl (5 mg, 0.025 mmol), HOBt H₂O (4 mg, 0.025 mmol), and DIPEA (0.016 mL, 0.092 mmol) was added and reacted overnight at room temperature. After completion of the reaction, it was diluted with water, extracted with EtOAc, and the residue was dried with MgSO₄, filtered and concentrated, and separated and purified by silica gel column chromatography (DCM/MeOH=6%) to obtain a yellow solid, 2-((4-fluorophenyl)amino)-2-oxo-1-phenylethyl 6-(1-((S)-1-acetylpyrrolidin-3-yl)-1H-pyrazol-4-yl)-3-aminopyrazine-2-carboxylate (10 mg, 0.018 mmol, 79%).

### Synthesis of TDH-229

The desired compound 2-((4-fluorophenyl)amino)-2-oxo-1-phenylethyl 6-(1-((R)-1-acetylpyrrolidin-3-yl)-1H-pyrazol-4-yl)-3-aminopyrazine-2-carboxylate was obtained in the same way as TDH-228 except that (R)-3-(4-(4,4,5,5-tetramethyl-1,3,2-dioxaborolan-2-yl)-1H-pyrazol-1-yl)pyrrolidine-1-carboxylate was used instead of (S)-3-(4-(4,4,5,5-tetramethyl-1,3,2-dioxaborolan-2-yl)-1H-pyrazol-1-yl)pyrrolidine-1-carboxylate in step 1 of the synthesis for TDH-228.

### Synthesis of (R)-2-((4-fluorophenyl)amino)-2-oxo-1-phenylethyl 6-amino-1′-(6-(2-((2-(2,6-dioxopiperidin-3-yl)-1,3-dioxoisoindolin-5-yl)amino)acetamido)hexanoyl)-1′,2′,3′,6′-tetrahydro-[3,4′-bipyridine]-5-carboxylate [TDH-072]

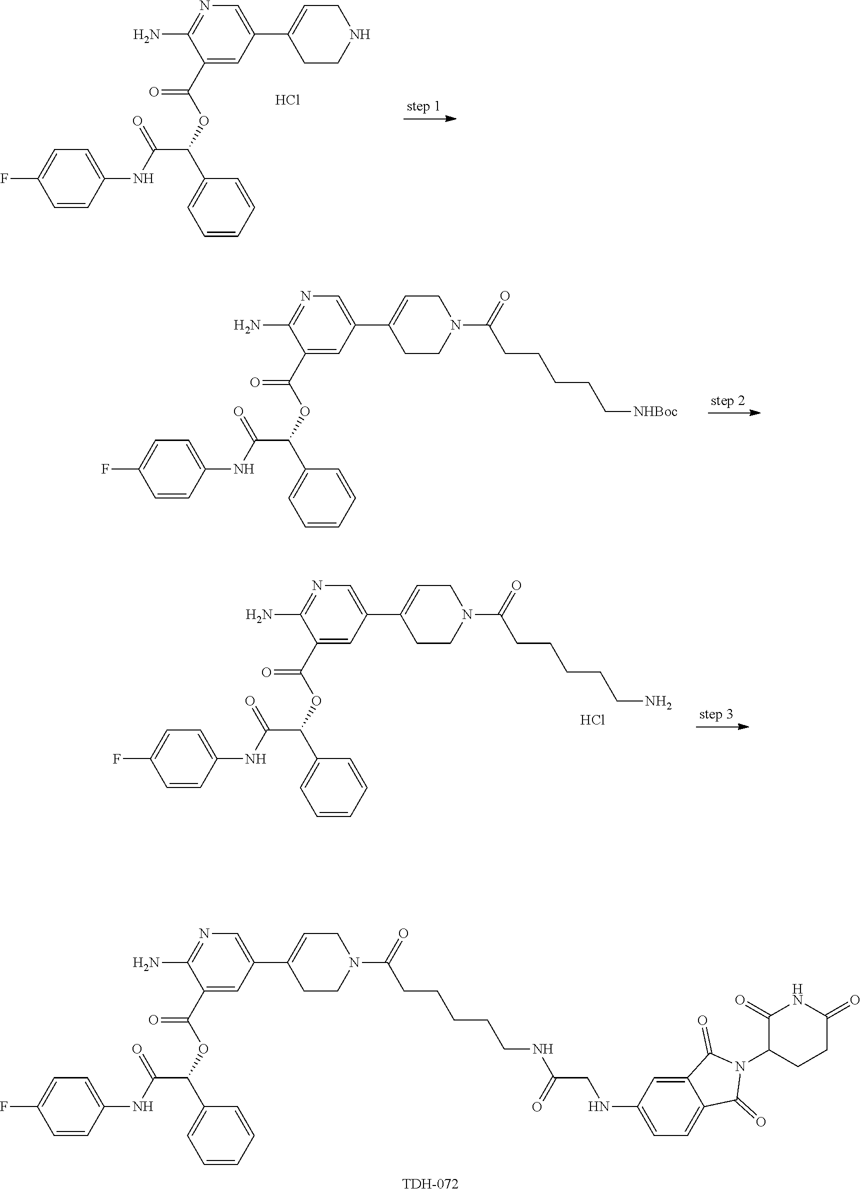 [SMILES: Nc1ncc(C2=CCNCC2)cc1C(=O)O[C@@H](C(=O)Nc1ccc(F)cc1)c1ccccc1 ; Cl ; CC(C)(C)OC(=O)NCCCCCC(=O)N1CC=C(c2cnc(N)c(C(=O)O[C@@H](C(=O)Nc3ccc(F)cc3)c3ccccc3)c2)CC1 ; NCCCCCC(=O)N1CC=C(c2cnc(N)c(C(=O)O[C@@H](C(=O)Nc3ccc(F)cc3)c3ccccc3)c2)CC1 ; Cl ; Nc1ncc(C2=CCN(C(=O)CCCCCNC(=O)CNc3ccc4c(c3)C(=O)N(C3CCC(=O)NC3=O)C4=O)CC2)cc1C(=O)O[C@@H](C(=O)Nc1ccc(F)cc1)c1ccccc1]

### Synthesis of (R)-2-((4-fluorophenyl)amino)-2-oxo-1-phenylethyl 6-amino-1′-(6-((tert-butoxycarbonyl)amino)hexanoyl)-1′,2′,3′,6′-tetrahydro-[3,4′-bipyridine]-5-carboxylate (step 1)

(R)-2-((4-fluorophenyl)amino)-2-oxo-1-phenylethyl 6-amino-1′,2′,3′,6′-tetrahydro-[3,4′-bipyridine]-5-carboxylate hydrochloride (104 mg, 0.22 mmol, 1 eq.), 6-((tert-butoxycarbonyl)amino)hexanoic acid (50 mg, 0.22 mmol, 1 eq.), HATU (90 mg, 0.24 mmol, 1.1 eq.), and DIPEA (0.19 mL, 1.1 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain the desired (R)-2-((4-fluorophenyl)amino)-2-oxo-1-phenylethyl 6-amino-1′-(6-((tert-butoxycarbonyl)amino)hexanoyl)-1′,2′,3′,6′-tetrahydro-[3,4′-bipyridine]-5-carboxylate (130 mg, 90%).

¹H NMR (400 MHz, CDCl₃) δ 8.36-8.25 (m, 1H), 8.22-8.09 (m, 2H), 7.65-7.57 (m, 2H), 7.52-7.39 (m, 5H), 6.98 (t, J=8.6 Hz, 2H), 6.44 (s, 2H), 6.35 (s, 1H), 5.94 (d, J=20.5 Hz, 1H), 4.60 (s, 1H), 4.22 (s, 1H), 4.13-4.08 (m, 1H), 3.85-3.76 (m, 1H), 3.72-3.63 (m, 1H), 3.19-3.10 (m, 2H), 2.59-2.46 (m, 2H), 2.42-2.29 (m, 2H), 1.79 (s, 1H), 1.73-1.61 (m, 2H), 1.56-1.47 (m, 2H), 1.46-1.41 (m, 9H), 1.40-1.33 (m, 2H).

### Synthesis of (R)-2-((4-fluorophenyl)amino)-2-oxo-1-phenylethyl 6-amino-1′-(6-aminohexanoyl)-1′,2′,3′,6′-tetrahydro-[3,4′-bipyridine]-5-carboxylate hydrochloride (step 2)

(R)-2-((4-fluorophenyl)amino)-2-oxo-1-phenylethyl 6-amino-1′-(6-((tert-butoxycarbonyl)amino)hexanoyl)-1′,2′,3′,6′-tetrahydro-[3,4′-bipyridine]-5-carboxylate (130 mg, 0.20 mmol) was dissolved in DCM (1.0 mL). Then, 4 N HCl in dioxane (0.5 mL) was slowly added, and reacted at room temperature for 1 hour. After completion of the reaction, the precipitate was filtered to obtain the desired (R)-2-((4-fluorophenyl)amino)-2-oxo-1-phenylethyl 6-amino-1′-(6-aminohexanoyl)-1′,2′,3′,6′-tetrahydro-[3,4′-bipyridine]-5-carboxylate hydrochloride (120 mg).

### Synthesis of (R)-2-((4-fluorophenyl)amino)-2-oxo-1-phenylethyl 6-amino-1′-(6-(2-((2-(2,6-dioxopiperidin-3-yl)-1,3-dioxoisoindolin-5-yl)amino)acetamido)hexanoyl)-1′,2′,3′,6′-tetrahydro-[3,4′-bipyridine]-5-carboxylate (TDH-072, step 3)

(R)-2-((4-fluorophenyl)amino)-2-oxo-1-phenylethyl 6-amino-1′-(6-aminohexanoyl)-1′,2′,3′,6′-tetrahydro-[3,4′-bipyridine]-5-carboxylate hydrochloride (25 mg, 0.042 mmol, 1 eq.), (2-(2,6-dioxopiperidin-3-yl)-1,3-dioxoisoindolin-5-yl)glycine (18 mg, 0.042 mmol, 1 eq.), HATU (18 mg, 0.046 mmol, 1.1 eq.), and DIPEA (0.04 mL, 0.21 mmol, 4 eq.) were dissolved in DMF (1.0 mL) and reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain the desired (R)-2-((4-fluorophenyl)amino)-2-oxo-1-phenylethyl 6-amino-1′-(6-(2-((2-(2,6-dioxopiperidin-3-yl)-1,3-dioxoisoindolin-5-yl)amino)acetamido)hexanoyl)-1′,2′,3′,6′-tetrahydro-[3,4′-bipyridine]-5-carboxylate (15 mg, 42%).

### Synthesis of (R)-2-((4-fluorophenyl)amino)-2-oxo-1-phenylethyl 6-amino-1′-(6-((2-(2,6-dioxopiperidin-3-yl)-1,3-dioxoisoindolin-4-yl)amino)hexanoyl)-1′,2′,3′,6′-tetrahydro-[3,4′-bipyridine]-5-carboxylate [TDH-073]

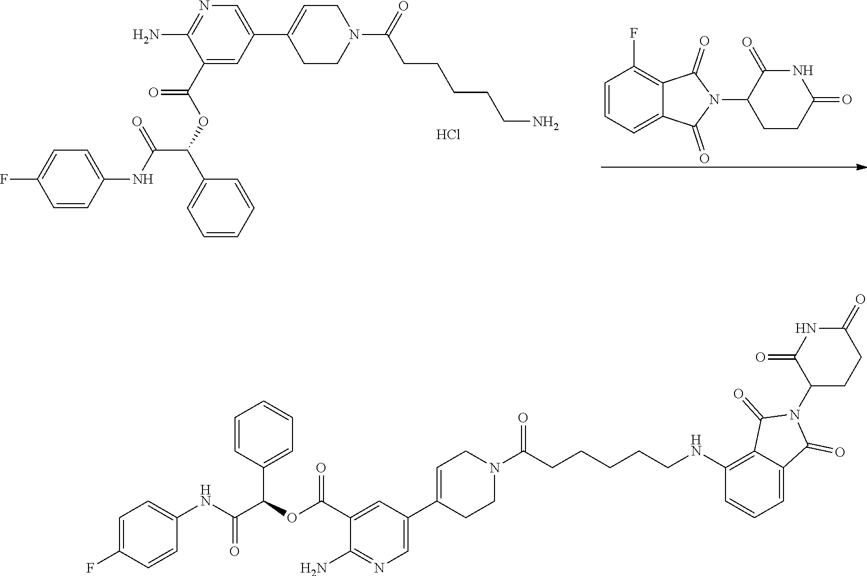 [SMILES: NCCCCCC(=O)N1CC=C(c2cnc(N)c(C(=O)O[C@@H](C(=O)Nc3ccc(F)cc3)c3ccccc3)c2)CC1 ; Cl ; O=C1CCC(N2C(=O)c3cccc(F)c3C2=O)C(=O)N1 ; Nc1ncc(C2=CCN(C(=O)CCCCCNc3cccc4c3C(=O)N(C3CCC(=O)NC3=O)C4=O)CC2)cc1C(=O)O[C@@H](C(=O)Nc1ccc(F)cc1)c1ccccc1]

(R)-2-((4-fluorophenyl)amino)-2-oxo-1-phenylethyl 6-amino-1′-(6-aminohexanoyl)-1′,2′,3′,6′-tetrahydro-[3,4′-bipyridine]-5-carboxylate hydrochloride 24 (25 mg, 0.042 mmol, 1 eq.), 4-fluorothalidomide (12 mg, 0.042 mmol, 1 eq.), and DIPEA (0.04 mL, 0.21 mmol, 5 eq.) were dissolved in DMSO (1.0 mL) and reacted overnight at 90° C. After completion of the reaction, it was diluted with water and extracted with EA. The combined organic layer was washed with brine, and the residual water was removed through anhydrous Na₂SO₄, followed by filtration. The organic layer was concentrated under reduced pressure and separated by a column to obtain the desired (R)-2-((4-fluorophenyl)amino)-2-oxo-1-phenylethyl 6-amino-1′-(6-((2-(2,6-dioxopiperidin-3-yl)-1,3-dioxoisoindolin-4-yl)amino)hexanoyl)-1′,2′,3′,6′-tetrahydro-[3,4′-bipyridine]-5-carboxylate (2 mg, 6%).

### Synthesis of (R)-2-((4-fluorophenyl)amino)-2-oxo-1-phenylethyl 3-amino-6-(1-(1-(6-(2-((2-(2,6-dioxopiperidin-3-yl)-1,3-dioxoisoindolin-5-yl)oxy)acetamido)hexyl)piperidin-4-yl)-1H-pyrazol-4-yl)pyrazine-2-carboxylate [TDH-165]

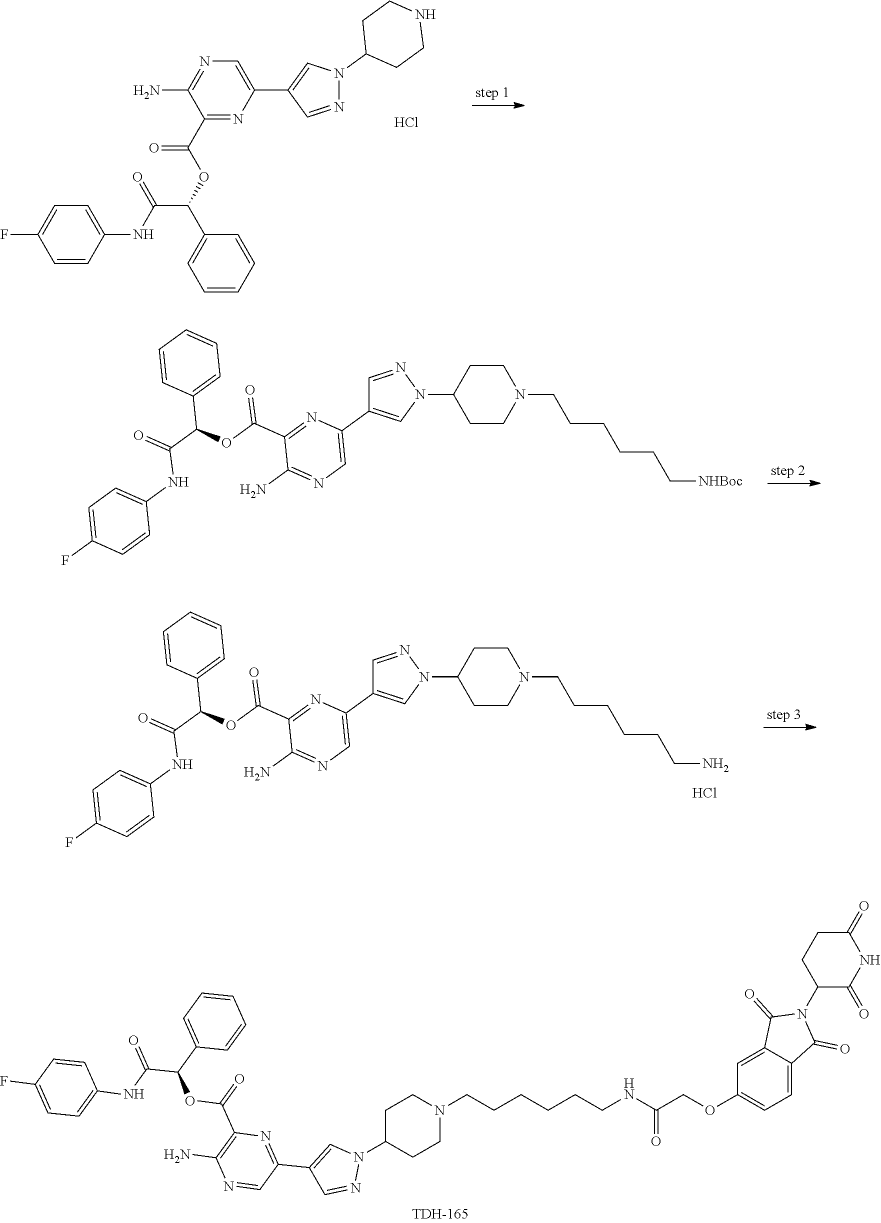

(R)-2-((4-fluorophenyl)amino)-2-oxo-1-phenylethyl 3-amino-6-(1-(1-(6-((tert-butoxycarbonyl)amino)hexyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylatehydrochloride (400 mg, 0.78 mmol, 1 eq.), 6-(Boc-amino)hexyl bromide (0.19 mL, 0.78 mmol, 1 eq.), and K₂CO₃ (322 mg, 2.3 mmol, 3.0 eq.) were dissolved with DMF (3.0 mL), and reacted overnight at 70° C. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain the desired (R)-2-((4-fluorophenyl)amino)-2-oxo-1-phenylethyl 3-amino-6-(1-(1-(6-((tert-butoxycarbonyl) amino)hexyl)piperidin-4-yl)-1H-pyrazol-4-yl)pyrazine-2-carboxylate (512 mg, 92%).

¹H NMR (400 MHz, CDCl₃) δ 8.94 (s, 1H), 8.54 (s, 1H), 7.95 (s, 1H), 7.70-7.63 (m, 2H), 7.61-7.53 (m, 2H), 7.51-7.40 (m, 3H), 7.06 (t, J=8.5 Hz, 2H), 6.44 (s, 1H), 6.33 (s, 2H), 4.62-4.46 (m, 1H), 4.34-4.18 (m, 1H), 3.21-3.02 (m, 4H), 2.56-2.38 (m, 2H), 2.32-1.99 (m, 6H), 1.68-1.44 (m, 13H), 1.41-1.33 (m, 4H).

### (R)-2-((4-fluorophenyl)amino)-2-oxo-1-phenylethyl 3-amino-6-(1-(1-(6-aminohexyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-(6-((tert-butoxycarbonyl)amino)hexyl)piperidin-4-yl)-1H-pyrazol-4-yl)pyrazine-2-carboxylate (512 mg, 0.72 mmol) was dissolved in DCM (1.5 mL). Then, 4 N HCl in dioxane (1.0 mL) was slowly added, and reacted at room temperature for 1 hour. After completion of the reaction, the precipitate was filtered to obtain the desired (R)-2-((4-fluorophenyl)amino)-2-oxo-1-phenylethyl 3-amino-6-(1-(1-(6-aminohexyl)piperidin-4-yl)-1H-pyrazol-4-yl)pyrazine-2-carboxylate hydrochloride (510 mg).

### (R)-2-((4-fluorophenyl)amino)-2-oxo-1-phenylethyl 3-amino-6-(1-(1-(6-(2-((2-(2,6-dioxopiperidin-3-yl)-1,3-dioxoisoindolin-5-yl)oxy)acetamido)hexyl)piperidin-4-yl)-1H-pyrazol-4-yl)pyrazine-2-carboxylate (TDH-165, step 3)

(R)-2-((4-fluorophenyl)amino)-2-oxo-1-phenylethyl 3-amino-6-(1-(1-(6-aminohexyl)piperidin-4-yl)-1H-pyrazol-4-yl)pyrazine-2-carboxylate hydrochloride (20 mg, 0.031 mmol, 1 eq.), 2-((2-(2,6-dioxopiperidin-3-yl)-1,3-dioxoisoindolin-5-yl)oxy)acetic acid (10 mg, 0.031 mmol, 1 eq.), EDCI-HCl (6 mg, 0.034 mmol, 1.1 eq.), HOBt (5 mg, 0.034 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain the desired (R)-2-((4-fluorophenyl)amino)-2-oxo-1-phenylethyl 3-amino-6-(1-(1-(6-(2-((2-(2,6-dioxopiperidin-3-yl)-1,3-dioxoisoindolin-5-yl)oxy)acetamido)hexyl)piperidin-4-yl)-1H-pyrazol-4-yl)pyrazine-2-carboxylate (16 mg, 56%).

### Synthesis of (R)-2-((4-fluorophenyl)amino)-2-oxo-1-phenylethyl 3-amino-6-(1-(1-(6-((2-(2,6-dioxopiperidin-3-yl)-1,3-dioxoisoindolin-4-yl)amino)hexyl)piperidin-4-yl)-1H-pyrazol-4-yl)pyrazine-2-carboxylate [TDH-119]

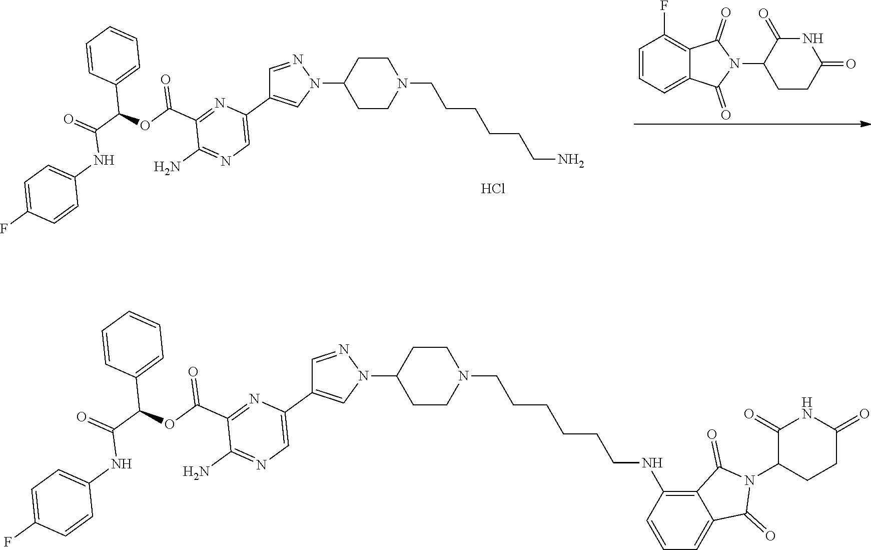 [SMILES: NCCCCCCN1CCC(n2cc(-c3cnc(N)c(C(=O)O[C@@H](C(=O)Nc4ccc(F)cc4)c4ccccc4)n3)cn2)CC1 ; Cl ; O=C1CCC(N2C(=O)c3cccc(F)c3C2=O)C(=O)N1 ; Nc1ncc(-c2cnn(C3CCN(CCCCCCNc4cccc5c4C(=O)N(C4CCC(=O)NC4=O)C5=O)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(6-aminohexyl)piperidin-4-yl)-1H-pyrazol-4-yl)pyrazine-2-carboxylate hydrochloride (25 mg, 0.042 mmol, 1 eq.), 4-fluorothalidomide (11 mg, 0.042 mmol, 1 eq.), and DIPEA (0.04 mL, 0.21 mmol, 5 eq.) were dissolved in DMSO (1.0 mL), and reacted overnight at 90° C. After completion of the reaction, it was diluted with water and extracted with EA. The combined organic layer was washed with brine, and the residual water was removed through anhydrous Na₂SO₄, followed by filtration. The organic layer was concentrated under reduced pressure and separated by a column to obtain the desired (R)-2-((4-fluorophenyl)amino)-2-oxo-1-phenylethyl 3-amino-6-(1-(1-(6-((2-(2,6-dioxopiperidin-3-yl)-1,3-dioxoisoindolin-4-yl)amino)hexyl)piperidin-4-yl)-1H-pyrazol-4-yl)pyrazine-2-carboxylate (7 mg, 22%).

### Synthesis of (R)-2-((4-fluorophenyl)amino)-2-oxo-1-phenylethyl 3-amino-6-(1-(1-(2-(2-(2-(2-((2-(2,6-dioxopiperidin-3-yl)-1,3-dioxoisoindolin-4-yl)oxy)acetamido)ethoxy)ethoxy)acetyl)piperidin-4-yl)-1H-pyrazol-4-yl)pyrazine-2-carboxylate [TDH-189]

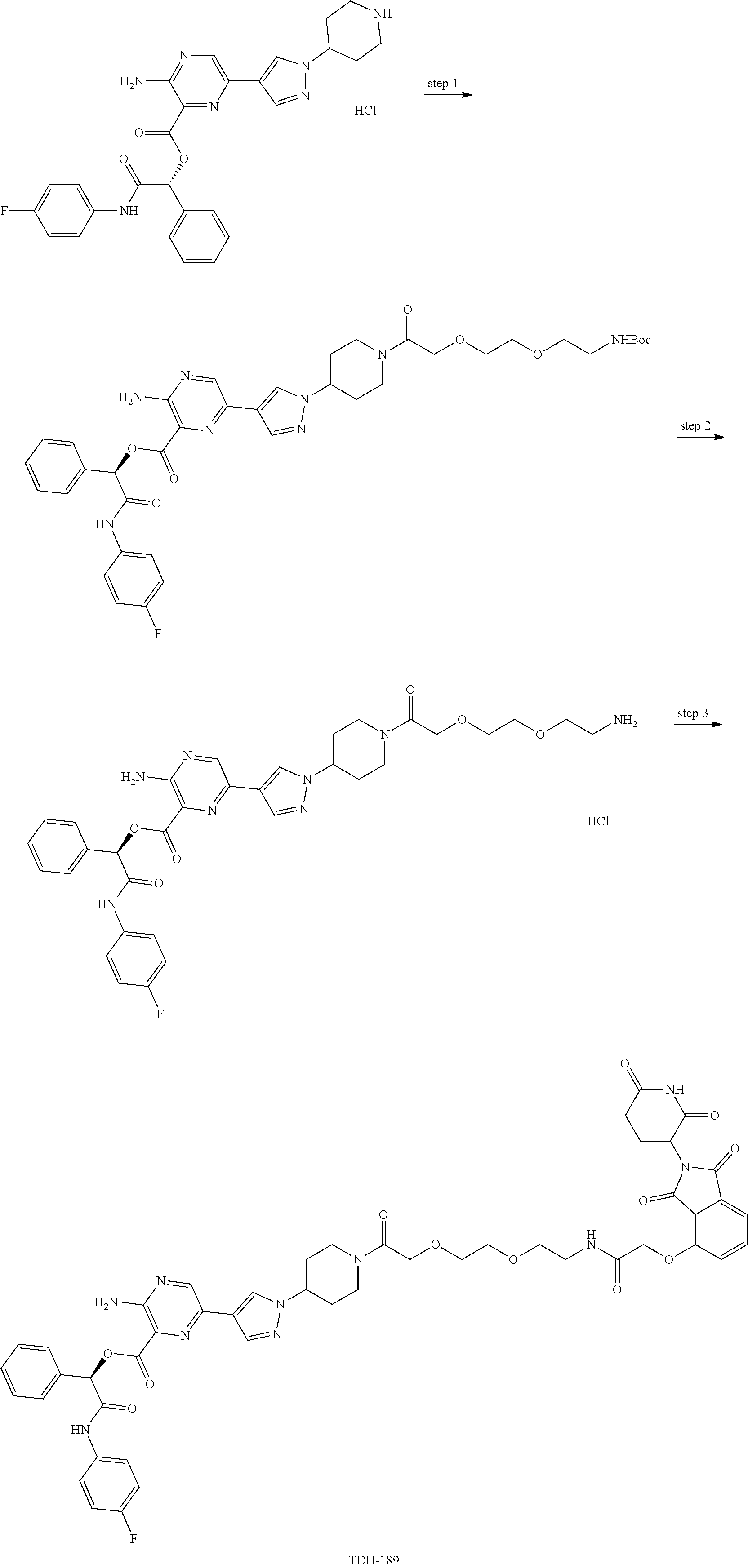 [SMILES: Nc1ncc(-c2cnn(C3CCNCC3)c2)nc1C(=O)O[C@@H](C(=O)Nc1ccc(F)cc1)c1ccccc1 ; Cl ; CC(C)(C)OC(=O)NCCOCCOCC(=O)N1CCC(n2cc(-c3cnc(N)c(C(=O)O[C@@H](C(=O)Nc4ccc(F)cc4)c4ccccc4)n3)cn2)CC1 ; NCCOCCOCC(=O)N1CCC(n2cc(-c3cnc(N)c(C(=O)O[C@@H](C(=O)Nc4ccc(F)cc4)c4ccccc4)n3)cn2)CC1 ; Cl ; Nc1ncc(-c2cnn(C3CCN(C(=O)COCCOCCNC(=O)COc4cccc5c4C(=O)N(C4CCC(=O)NC4=O)C5=O)CC3)c2)nc1C(=O)O[C@@H](C(=O)Nc1ccc(F)cc1)c1ccccc1]

### (R)-2-((4-fluorophenyl)amino)-2-oxo-1-phenylethyl 3-amino-6-(1-(1-(2,2-dimethyl-4-oxo-3,8,11-trioxa-5-azatridecan-13-oyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylate hydrochloride (200 mg, 0.39 mmol, 1 eq.), 2,2-dimethyl-4-oxo-3,8,11-trioxa-5-azatridecan-13-oic acid (102 mg, 0.39 mmol, 1.0 eq.), EDCI-HCl (82 mg, 0.43 mmol, 1.1 eq.), HOBt (65 mg, 0.43 mmol, 1.1 eq.), and DIPEA (0.3 mL, 1.9 mmol, 5 eq.) were dissolved in DMF (1.5 mL), and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain the desired (R)-2-((4-fluorophenyl)amino)-2-oxo-1-phenylethyl 3-amino-6-(1-(1-(2,2-dimethyl-4-oxo-3,8,11-trioxa-5-azatridecan-13-oyl)piperidin-4-yl)-1H-pyrazol-4-yl)pyrazine-2-carboxylate (251 mg).

### (R)-2-((4-fluorophenyl)amino)-2-oxo-1-phenylethyl 3-amino-6-(1-(1-(2-(2-(2-aminoethoxy)ethoxy)acetyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-(2,2-dimethyl-4-oxo-3,8,11-trioxa-5-azatridecan-13-oyl)piperidin-4-yl)-1H-pyrazol-4-yl)pyrazine-2-carboxylate (200 mg, 0.26 mmol) was dissolved in DCM (1.5 mL). Then, 4 N HCl in dioxane (1.0 mL) was slowly added, and the mixture was reacted at room temperature for 1 hour. After completion of the reaction, the precipitate was filtered to obtain the desired (R)-2-((4-fluorophenyl)amino)-2-oxo-1-phenylethyl 3-amino-6-(1-(1-(2-(2-(2-aminoethoxy)ethoxy)acetyl)piperidin-4-yl)-1H-pyrazol-4-yl)pyrazine-2-carboxylate hydrochloride (180 mg).

### (R)-2-((4-fluorophenyl)amino)-2-oxo-1-phenylethyl 3-amino-6-(1-(1-(2-(2-(2-(2-((2-(2,6-dioxopiperidin-3-yl)-1,3-dioxoisoindolin-4-yl)oxy)acetamido)ethoxy)ethoxy)acetyl)piperidin-4-yl)-1H-pyrazol-4-yl)pyrazine-2-carboxylate (TDH-189, step 3)

(R)-2-((4-fluorophenyl)amino)-2-oxo-1-phenylethyl 3-amino-6-(1-(1-(2-(2-(2-aminoethoxy)ethoxy)acetyl)piperidin-4-yl)-1H-pyrazol-4-yl)pyrazine-2-carboxylate hydrochloride (20 mg, 0.029 mmol, 1 eq.), 2-((2-(2,6-dioxopiperidin-3-yl)-1,3-dioxoisoindolin-4-yl)oxy)acetic acid (10 mg, 0.029 mmol, 1 eq.), EDCI-HCl (6 mg, 0.032 mmol, 1.1 eq.), HOBt (5 mg, 0.032 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain the desired (R)-2-((4-fluorophenyl)amino)-2-oxo-1-phenylethyl 3-amino-6-(1-(1-(2-(2-(2-(2-((2-(2,6-dioxopiperidin-3-yl)-1,3-dioxoisoindolin-4-yl)oxy)acetamido)ethoxy)ethoxy)acetyl)piperidin-4-yl)-1H-pyrazol-4-yl)pyrazine-2-carboxylate (17 mg, 60%).

### Synthesis of (R)-2-((4-fluorophenyl)amino)-2-oxo-1-phenylethyl 3-amino-6-(1-(1-(2-(2-(2-((2-(2,6-dioxopiperidin-3-yl)-1,3-dioxoisoindolin-4-yl)amino)ethoxy)ethoxy)acetyl)piperidin-4-yl)-1H-pyrazol-4-yl)pyrazine-2-carboxylate [TDH-104]

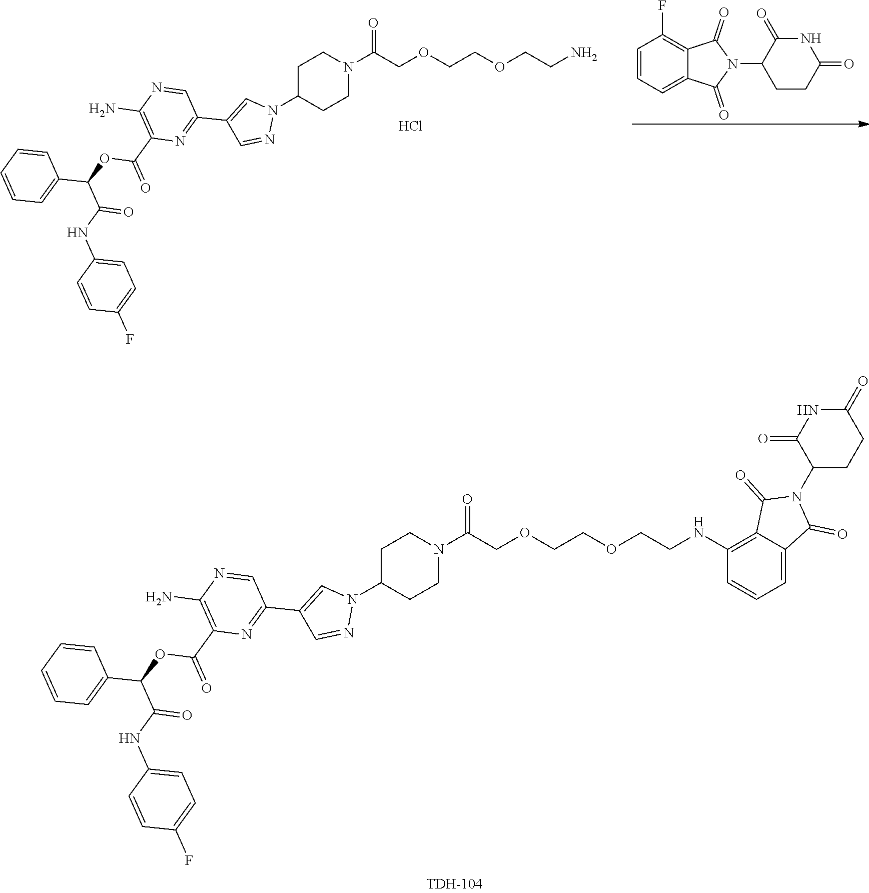 [SMILES: NCCOCCOCC(=O)N1CCC(n2cc(-c3cnc(N)c(C(=O)O[C@@H](C(=O)Nc4ccc(F)cc4)c4ccccc4)n3)cn2)CC1 ; Cl ; O=C1CCC(N2C(=O)c3cccc(F)c3C2=O)C(=O)N1 ; Nc1ncc(-c2cnn(C3CCN(C(=O)COCCOCCNc4cccc5c4C(=O)N(C4CCC(=O)NC4=O)C5=O)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(2-(2-aminoethoxy)ethoxy)acetyl)piperidin-4-yl)-1H-pyrazol-4-yl)pyrazine-2-carboxylate hydrochloride (20 mg, 0.029 mmol, 1 eq.), 4-fluorothalidomide (8 mg, 0.029 mmol, 1 eq.), and DIPEA (0.04 mL, 0.21 mmol, 5 eq.) were dissolved in DMSO (1.0 mL) and reacted at 90° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure and separated by a column to obtain (R)-2-((4-fluorophenyl)amino)-2-oxo-1-phenylethyl 3-amino-6-(1-(1-(2-(2-(2-((2-(2,6-dioxopiperidin-3-yl)-1,3-dioxoisoindolin-4-yl)amino)ethoxy)ethoxy)acetyl)piperidin-4-yl)-1H-pyrazol-4-yl)pyrazine-2-carboxylate (7 mg, 27%).

### Synthesis of (R)-2-((4-fluorophenyl)amino)-2-oxo-1-phenylethyl 3-amino-6-(1-(1-((1-(2-((2-(2,6-dioxopiperidin-3-yl)-1,3-dioxoisoindolin-4-yl)oxy)acetyl)piperidin-4-yl)methyl)piperidin-4-yl)-1H-pyrazol-4-yl)pyrazine-2-carboxylate [TDH-148]

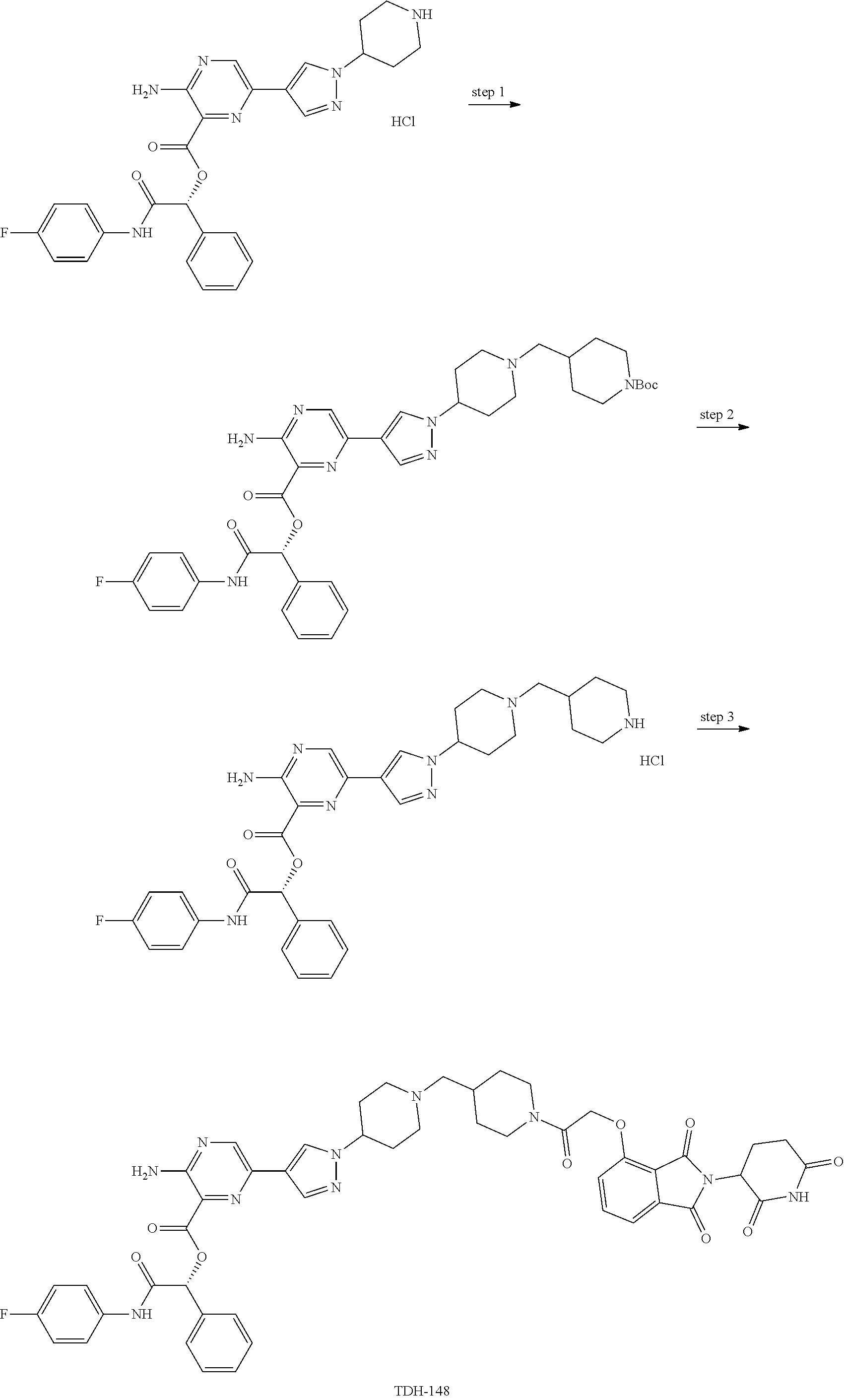 [SMILES: Nc1ncc(-c2cnn(C3CCNCC3)c2)nc1C(=O)O[C@@H](C(=O)Nc1ccc(F)cc1)c1ccccc1 ; Cl ; CC(C)(C)OC(=O)N1CCC(CN2CCC(n3cc(-c4cnc(N)c(C(=O)O[C@@H](C(=O)Nc5ccc(F)cc5)c5ccccc5)n4)cn3)CC2)CC1 ; Nc1ncc(-c2cnn(C3CCN(CC4CCNCC4)CC3)c2)nc1C(=O)O[C@@H](C(=O)Nc1ccc(F)cc1)c1ccccc1 ; Cl ; Nc1ncc(-c2cnn(C3CCN(CC4CCN(C(=O)COc5cccc6c5C(=O)N(C5CCC(=O)NC5=O)C6=O)CC4)CC3)c2)nc1C(=O)O[C@@H](C(=O)Nc1ccc(F)cc1)c1ccccc1]

### (R)-2-((4-fluorophenyl)amino)-2-oxo-1-phenylethyl 3-amino-6-(1-(1-((1-(tert-butoxycarbonyl)piperidin-4-yl)methyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylatehydrochloride (300 mg, 0.54 mol, 1 eq.) and K₂CO₃ (223 mg, 1.62 mmol, 3 eq.) were dissolved in DMF (10 mL), and the mixture was reacted at 70° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain the desired ((R)-2-((4-fluorophenyl)amino)-2-oxo-1-phenylethyl 3-amino-6-(1-(1-((1-(tert-butoxycarbonyl)piperidin-4-yl)methyl)piperidin-4-yl)-1H-pyrazol-4-yl)pyrazine-2-carboxylate (78 mg, 20%).

¹H NMR (300 MHz, Chloroform-d) δ 8.98 (s, 1H), 8.54 (s, 1H), 7.99 (d, J=0.7 Hz, 1H), 7.95 (d, J=6.2 Hz, 1H), 7.67 (td, J=7.5, 2.9 Hz, 2H), 7.62-7.54 (m, 2H), 7.49-7.39 (m, 3H), 7.05 (t, J=8.6 Hz, 2H), 6.44 (s, 1H), 6.33 (s, 2H), 4.18 (d, J=18.7 Hz, 3H), 3.02 (d, J=10.2 Hz, 2H), 2.72 (t, J=12.7 Hz, 2H), 2.30-1.94 (m, 10H), 1.84-1.62 (m, 6H), 1.48 (s, 11H), 1.24-1.01 (m, 3H).

### (R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (step 2)

((R)-2-((4-fluorophenyl)amino)-2-oxo-1-phenylethyl 3-amino-6-(1-(1-((1-(tert-butoxycarbonyl)piperidin-4-yl)methyl)piperidin-4-yl)-1H-pyrazol-4-yl)pyrazine-2-carboxylate (131 mg, 0.18 mmol) was dissolved in DCM (2.0 mL). Then, 4 N HCl in dioxane (1.0 mL) was slowly added, and reacted at room temperature for 1 hour. After completion of the reaction, the precipitate was filtered to obtain (R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (113 mg).

### (R)-2-((4-fluorophenyl)amino)-2-oxo-1-phenylethyl 3-amino-6-(1-(1-((1-(2-((2-(2,6-dioxopiperidin-3-yl)-1,3-dioxoisoindolin-4-yl)oxy)acetyl)piperidin-4-yl)methyl)piperidin-4-yl)-1H-pyrazol-4-yl)pyrazine-2-carboxylate (TDH-148, step 3)

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (25 mg, 0.040 mmol, 1 eq.), 2-((2-(2,6-dioxopiperidin-3-yl)-1,3-dioxoisoindolin-4-yl)oxy)acetic acid (7 mg, 0.020 mmol, 1.0 eq.), EDCI-HCl (8 mg, 0.044 mmol, 1.1 eq.), HOBt (6 mg, 0.044 mmol, 1.1 eq.), and DIPEA (0.03 mL, 0.17 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain the desired (R)-2-((4-fluorophenyl)amino)-2-oxo-1-phenylethyl 3-amino-6-(1-(1-((1-(2-((2-(2,6-dioxopiperidin-3-yl)-1,3-dioxoisoindolin-4-yl)oxy)acetyl)piperidin-4-yl)methyl)piperidin-4-yl)-1H-pyrazol-4-yl)pyrazine-2-carboxylate (11 mg, 35%).

### Synthesis of (R)-2-((4-fluorophenyl)amino)-2-oxo-1-phenylethyl 3-amino-6-(1-(1-((1-(2-(2,6-dioxopiperidin-3-yl)-1,3-dioxoisoindolin-4-yl)piperidin-4-yl)methyl)piperidin-4-yl)-1H-pyrazol-4-yl)pyrazine-2-carboxylate [TDH-149]

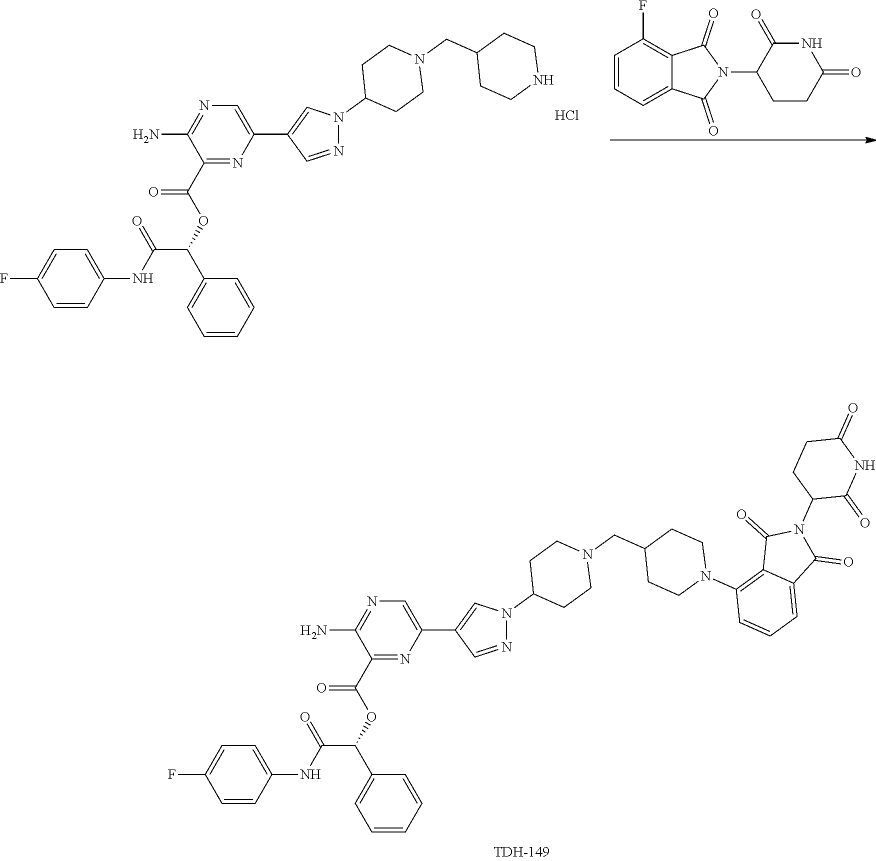 [SMILES: Nc1ncc(-c2cnn(C3CCN(CC4CCNCC4)CC3)c2)nc1C(=O)O[C@@H](C(=O)Nc1ccc(F)cc1)c1ccccc1 ; Cl ; O=C1CCC(N2C(=O)c3cccc(F)c3C2=O)C(=O)N1 ; Nc1ncc(-c2cnn(C3CCN(CC4CCN(c5cccc6c5C(=O)N(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (20 mg, 0.03 mmol, 1 eq.), 4-fluorothalidomide (9 mg, 0.03 mmol, 1 eq.), and DIPEA (0.04 mL, 0.15 mmol, 5 eq.) were dissolved in DMSO (1.0 mL) and reacted overnight at 90° C. After completion of the reaction, it was diluted with water and extracted with EA. The combined organic layer was washed with brine, and the residual water was removed through anhydrous Na₂SO₄, followed by filtration. The organic layer was concentrated under reduced pressure and separated by a column to obtain (R)-2-((4-fluorophenyl)amino)-2-oxo-1-phenylethyl 3-amino-6-(1-(1-((1-(2-(2,6-dioxopiperidin-3-yl)-1,3-dioxoisoindolin-4-yl)piperidin-4-yl)methyl)piperidin-4-yl)-1H-pyrazol-4-yl)pyrazine-2-carboxylate (4 mg, 14%).

### Synthesis of (R)-2-((4-fluorophenyl)amino)-2-oxo-1-phenylethyl 3-amino-6-(1-(1-(2-(2-(2-(2-((2-(2,6-dioxopiperidin-3-yl)-1,3-dioxoisoindolin-4-yl)oxy)acetamido)ethoxy)ethoxy)acetyl)piperidin-4-yl)-1H-pyrazol-4-yl)pyrazine-2-carboxylate [TDH-277]

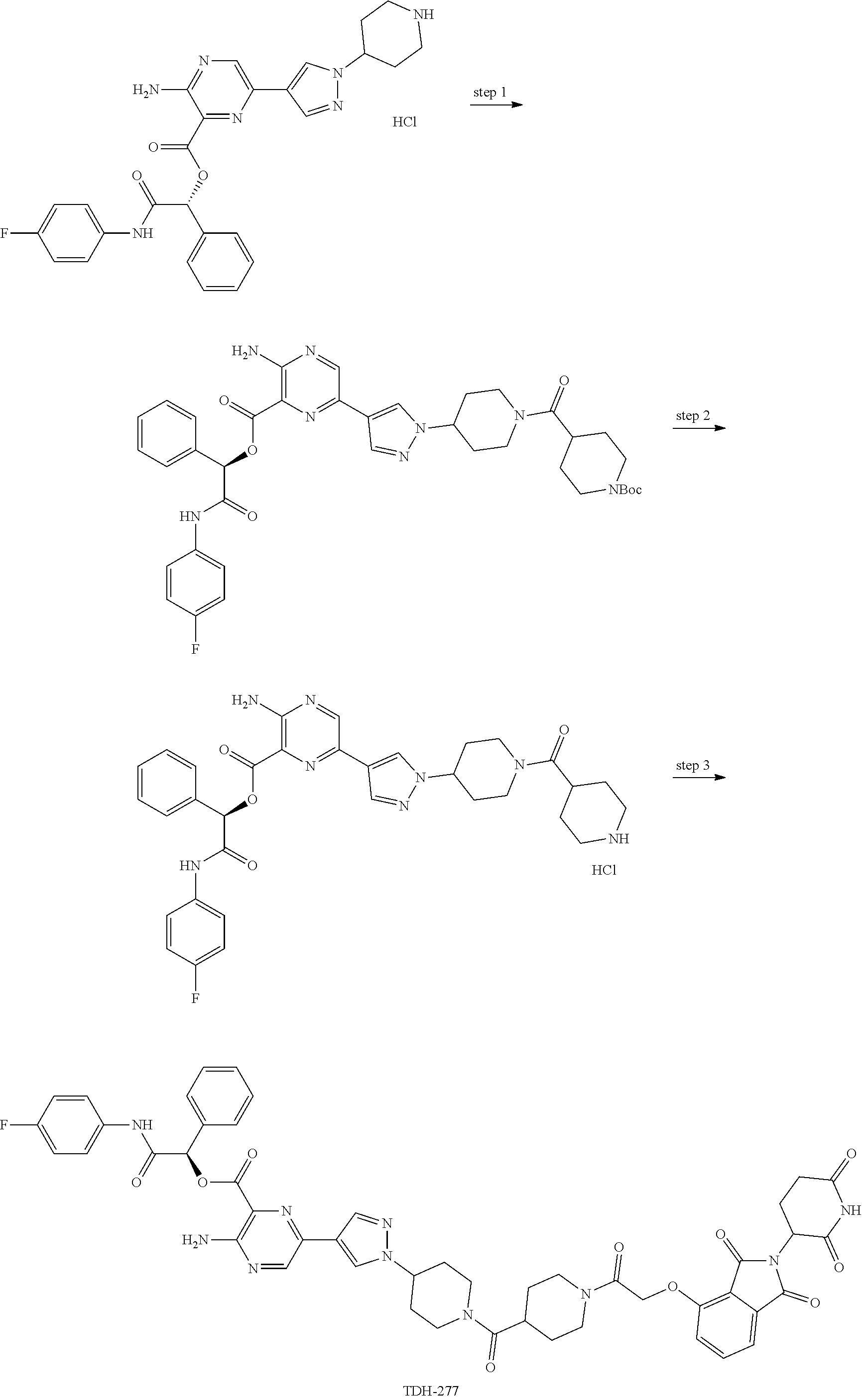 [SMILES: Nc1ncc(-c2cnn(C3CCNCC3)c2)nc1C(=O)O[C@@H](C(=O)Nc1ccc(F)cc1)c1ccccc1 ; Cl ; CC(C)(C)OC(=O)N1CCC(C(=O)N2CCC(n3cc(-c4cnc(N)c(C(=O)O[C@@H](C(=O)Nc5ccc(F)cc5)c5ccccc5)n4)cn3)CC2)CC1 ; Nc1ncc(-c2cnn(C3CCN(C(=O)C4CCNCC4)CC3)c2)nc1C(=O)O[C@@H](C(=O)Nc1ccc(F)cc1)c1ccccc1 ; Cl ; Nc1ncc(-c2cnn(C3CCN(C(=O)C4CCN(C(=O)COc5cccc6c5C(=O)N(C5CCC(=O)NC5=O)C6=O)CC4)CC3)c2)nc1C(=O)O[C@@H](C(=O)Nc1ccc(F)cc1)c1ccccc1]

### (R)-2-((4-fluorophenyl)amino)-2-oxo-1-phenylethyl 3-amino-6-(1-(1-(1-(tert-butoxycarbonyl)piperidine-4-carbonyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylatehydrochloride (250 mg, 0.45 mmol, 1 eq.), 1-(tert-butoxycarbonyl)piperidine-4-carboxylic acid (104 mg, 0.45 mmol, 1.0 eq.), EDCI-HCl (96 mg, 0.50 mmol, 1.1 eq.), HOBt (76 mg, 0.50 mmol, 1.1 eq.), and DIPEA (0.40 mL, 2.3 mmol, 5 eq.) were dissolved in DMF (1.8 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain the desired ((R)-2-((4-fluorophenyl)amino)-2-oxo-1-phenylethyl 3-amino-6-(1-(1-(1-(tert-butoxycarbonyl)piperidine-4-carbonyl)piperidin-4-yl)-1H-pyrazol-4-yl)pyrazine-2-carboxylate (339 mg, quant.).

¹H NMR (400 MHz, Chloroform-d) δ 8.96 (s, 1H), 8.49 (s, 1H), 7.98 (s, 1H), 7.87 (s, 1H), 7.66-7.60 (m, 2H), 7.55-7.49 (m, 2H), 7.45-7.36 (m, 3H), 7.03-6.95 (m, 2H), 6.42 (s, 1H), 6.35 (s, 2H), 4.84-4.71 (m, 1H), 4.44-4.35 (m, 1H), 4.22-4.02 (m, 4H), 3.30-3.18 (m, 1H), 2.84-2.72 (m, 4H), 2.69-2.60 (m, 1H), 2.34-2.14 (m, 3H), 2.07-1.89 (m, 3H), 1.45 (s, 9H).

### (R)-2-((4-fluorophenyl)amino)-2-oxo-1-phenylethyl 3-amino-6-(1-(1-(piperidine-4-carbonyl)piperidin-4-yl)-1H-pyrazol-4-yl)pyrazine-2-carboxylate hydrochloride (step 2)

((R)-2-((4-fluorophenyl)amino)-2-oxo-1-phenylethyl 3-amino-6-(1-(1-(1-(tert-butoxycarbonyl)piperidine-4-carbonyl)piperidin-4-yl)-1H-pyrazol-4-yl)pyrazine-2-carboxylate (339 mg, 0.47 mmol) was dissolved in DCM (2.0 mL), 4 N HCl in dioxane (1.0 mL) was slowly added, and reacted at room temperature for 1 hour. After the reaction was completed, the precipitate was filtered to obtain (R)-2-((4-fluorophenyl)amino)-2-oxo-1-phenylethyl 3-amino-6-(1-(1-(piperidine-4-carbonyl)piperidin-4-yl)-1H-pyrazol-4-yl)pyrazine-2-carboxylate hydrochloride (309 mg).

### (R)-2-((4-fluorophenyl)amino)-2-oxo-1-phenylethyl 3-amino-6-(1-(1-(1-(2-((2-(2,6-dioxopiperidin-3-yl)-1,3-dioxoisoindolin-4-yl)oxy)acetyl)piperidine-4-carbonyl)piperidin-4-yl)-1H-pyrazol-4-yl)pyrazine-2-carboxylate (TDH-277, step 3)

(R)-2-((4-fluorophenyl)amino)-2-oxo-1-phenylethyl 3-amino-6-(1-(1-(piperidine-4-carbonyl)piperidin-4-yl)-1H-pyrazol-4-yl)pyrazine-2-carboxylate hydrochloride (15 mg, 0.020 mmol, 1 eq.), 2-((2-(2,6-dioxopiperidin-3-yl)-1,3-dioxoisoindolin-4-yl)oxy)acetic acid (7 mg, 0.020 mmol, 1.0 eq.), EDCI-HCl (4 mg, 0.022 mmol, 1.1 eq.), HOBt (3 mg, 0.022 mmol, 1.1 eq.), and DIPEA (0.02 mL, 0.10 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain the desired (R)-2-((4-fluorophenyl)amino)-2-oxo-1-phenylethyl 3-amino-6-(1-(1-(1-(2-((2-(2,6-dioxopiperidin-3-yl)-1,3-dioxoisoindolin-4-yl)oxy)acetyl)piperidine-4-carbonyl)piperidin-4-yl)-1H-pyrazol-4-yl)pyrazine-2-carboxylate (11 mg, 58%).

### Synthesis of (R)-2-((4-fluorophenyl)amino)-2-oxo-1-phenylethyl 3-amino-6-(1-(1-(1-(2-(2,6-dioxopiperidin-3-yl)-1,3-dioxoisoindolin-4-yl)piperidine-4-carbonyl)piperidin-4-yl)-1H-pyrazol-4-yl)pyrazine-2-carboxylate [TDH-279]

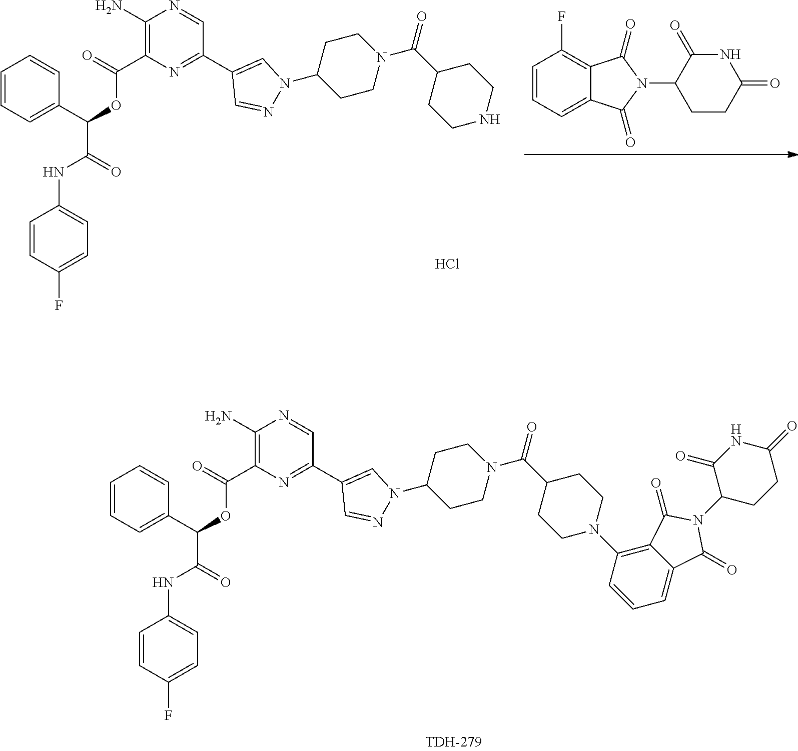

(R)-2-((4-fluorophenyl)amino)-2-oxo-1-phenylethyl 3-amino-6-(1-(1-(piperidine-4-carbonyl)piperidin-4-yl)-1H-pyrazol-4-yl)pyrazine-2-carboxylate hydrochloride (20 mg, 0.03 mmol, 1 eq.), 4-fluorothalidomide (8 mg, 0.03 mmol, 1 eq.), and DIPEA (0.04 mL, 0.15 mmol, 5 eq.) were dissolved in DMSO (1.0 mL) and reacted overnight at 90° C. After completion of the reaction, it was diluted with water and extracted with EA. The combined organic layer was washed with brine, and the residual water was removed through anhydrous Na₂SO₄, followed by filtration. The organic layer was concentrated under reduced pressure and separated by a column to obtain (R)-2-((4-fluorophenyl)amino)-2-oxo-1-phenylethyl 3-amino-6-(1-(1-(1-(2-(2,6-dioxopiperidin-3-yl)-1,3-dioxoisoindolin-4-yl)piperidine-4-carbonyl)piperidin-4-yl)-1H-pyrazol-4-yl)pyrazine-2-carboxylate (17 mg, 96%).

### Synthesis of (R)-2-((4-fluorophenyl)amino)-2-oxo-1-phenylethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate [TDH-358]

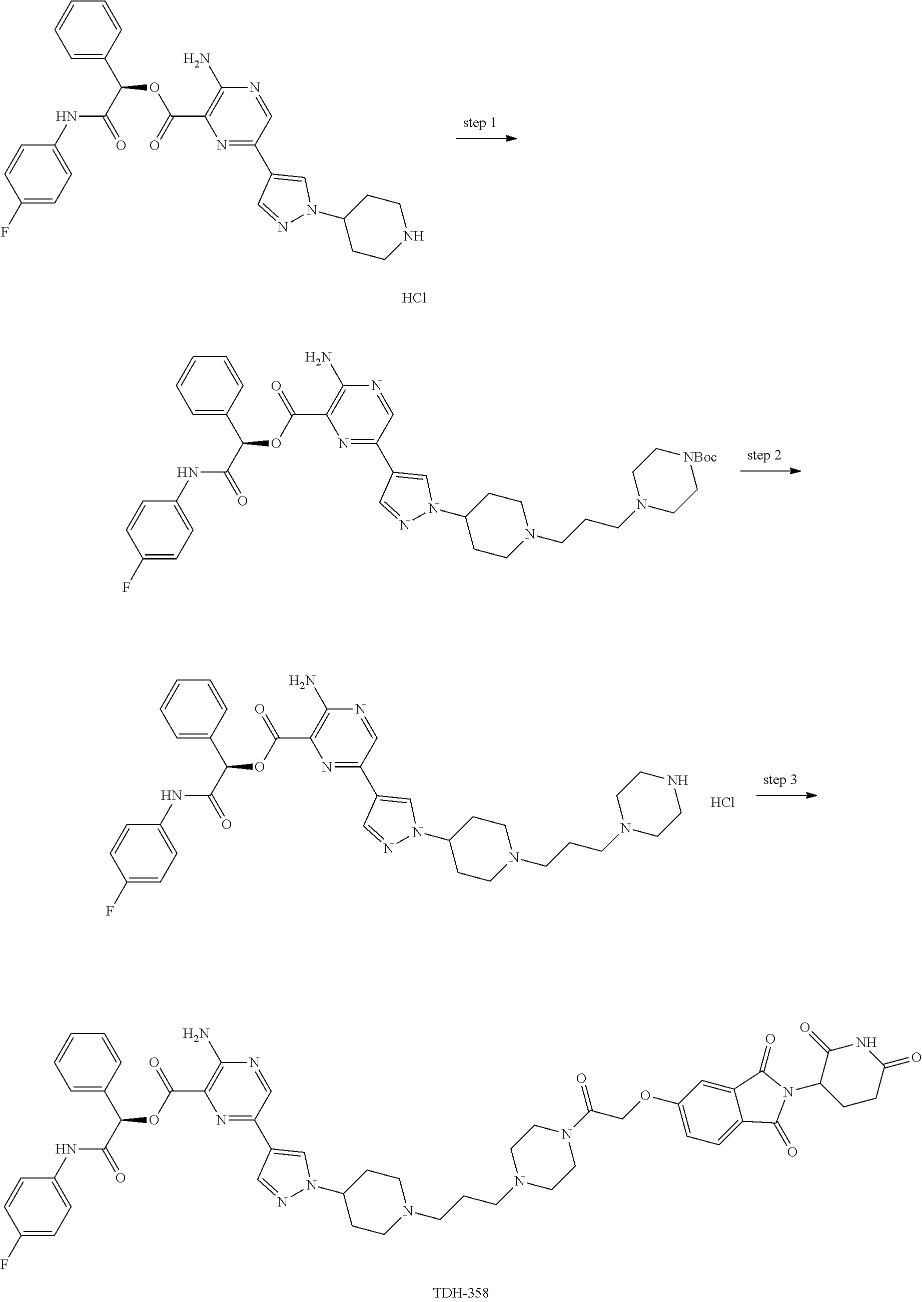 [SMILES: Nc1ncc(-c2cnn(C3CCNCC3)c2)nc1C(=O)O[C@@H](C(=O)Nc1ccc(F)cc1)c1ccccc1 ; Cl ; CC(C)(C)OC(=O)N1CCN(CCCN2CCC(n3cc(-c4cnc(N)c(C(=O)O[C@@H](C(=O)Nc5ccc(F)cc5)c5ccccc5)n4)cn3)CC2)CC1 ; Nc1ncc(-c2cnn(C3CCN(CCCN4CCNCC4)CC3)c2)nc1C(=O)O[C@@H](C(=O)Nc1ccc(F)cc1)c1ccccc1 ; Cl ; Nc1ncc(-c2cnn(C3CCN(CCCN4CCN(C(=O)COc5ccc6c(c5)C(=O)N(C5CCC(=O)NC5=O)C6=O)CC4)CC3)c2)nc1C(=O)O[C@@H](C(=O)Nc1ccc(F)cc1)c1ccccc1]

### (R)-2-((4-fluorophenyl)amino)-2-oxo-1-phenylethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylate hydrochloride (103 mg, 0.20 mmol, 1 eq.), tert-butyl 4-(3-iodopropyl)piperazine-1-carboxylate (105 mg, 0.30 mmol, 1.5 eq.), and K₂CO₃ (56 mg, 040 mmol, 2.0 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain (R)-2-((4-fluorophenyl)amino)-2-oxo-1-phenylethyl 3-amino-6-(1-(1-(3-(4-(tert-butoxycarbonyl) Piperazin-1-yl)propyl)piperidin-4-yl)-1H-pyrazol-4-yl)pyrazine-2-carboxylate (45 mg).

### (R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (165 mg, 0.49 mmol) was dissolved in DCM (2.0 mL), and 4 N HCl in dioxane (1.0 mL) was slowly added. After that, it was reacted at room temperature for 1 hour. After completion of the reaction, the precipitate was filtered to obtain the desired (R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (155 mg).

### (R)-2-((4-fluorophenyl)amino)-2-oxo-1-phenylethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate (TDH-358, step 3)

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (40 mg, 0.059 mmol, 1 eq.), 2-((2-(2,6-dioxopiperidin-3-yl)-1,3-dioxoisoindolin-5-yl)oxy)acetic acid (20 mg, 0.059 mmol, 1.0 eq.), EDCI-HCl (13 mg, 0.065 mmol, 1.1 eq.), HOBt (10 mg, 0.065 mmol, 1.1 eq.), and DIPEA (0.03 mL, 0.12 mmol, 5 eq.) were dissolved in DMF (1.0 mL), and the mixture was reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure to obtain the desired (R)-2-((4-fluorophenyl)amino)-2-oxo-1-phenylethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate (35 mg, 62%).

### Synthesis of ((R)-2-(4-methylpiperazin-1-yl)-2-oxo-1-phenylethyl 3-amino-6-(1-(1-(tert-butoxycarbonyl)piperidin-4-yl)-1H-pyrazol-4-yl)pyrazine-2-carboxylate hydrochloride (TDH-455)

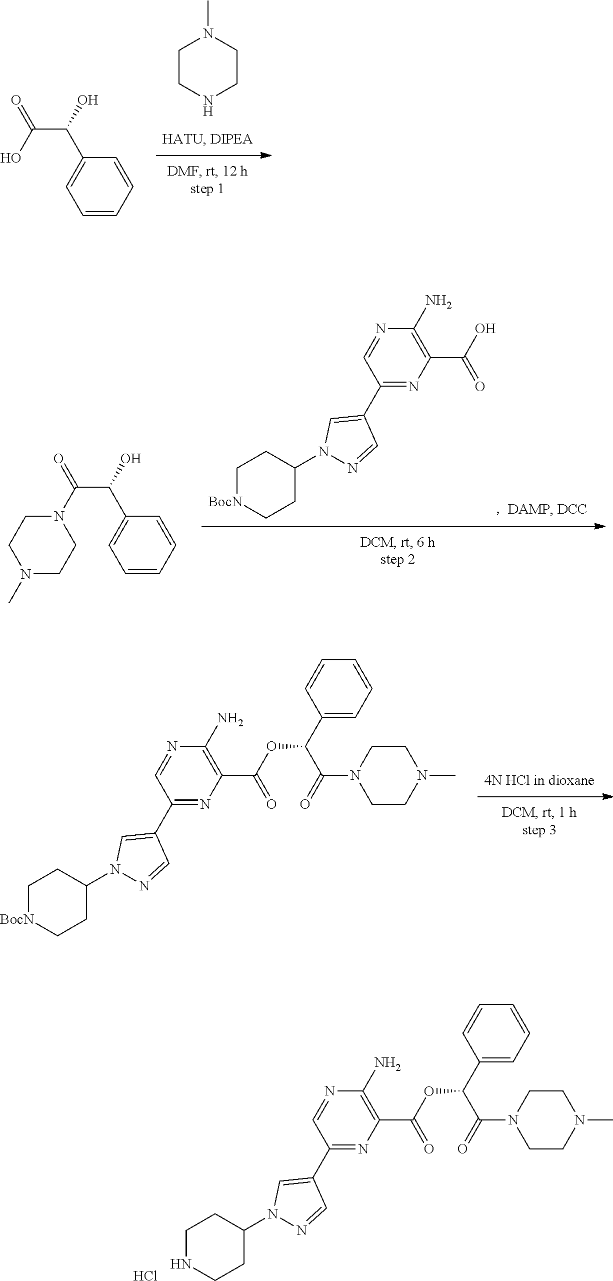

### Synthesis of (R)-2-hydroxy-1-(4-methylpiperazin-1-yl)-2-phenylethan-1-one (step 1)

(R)-2-hydroxy-2-phenylacetic acid (304 mg, 2.0 mmol) was dissolved in DMF (8 mL). Then, DIPEA (1.7 mL, 10 mmol) and HATU (1.14 g, 3.0 mmol) was added thereto, and stirred for 20 minutes. 1-Methylpiperazine (0.3 mL, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired light orange solid, (R)-2-hydroxy-1-(4-methylpiperazin-1-yl)-2-phenylethan-1-one (421 mg, 1.8 mmol, 90%).

¹H NMR (300 MHz, CDCl₃) δ 7.46-7.30 (m, 5H), 5.22 (s, 1H), 4.78 (s, 1H), 3.90-3.79 (m, 1H), 3.69-3.57 (m, 1H), 3.37-3.26 (m, 1H), 3.26-3.16 (m, 1H), 2.52-2.42 (m, 1H), 2.32-2.23 (m, 2H), 2.22 (s, 3H), 1.83-1.73 (m, 1H).

### Synthesis of (R)-2-(4-methylpiperazin-1-yl)-2-oxo-1-phenylethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)-2-hydroxy-1-(4-methylpiperazin-1-yl)-2-phenylethan-1-one (37 mg, 0.15 mmol), DCC (32 mg, 0.15 mmol), and DMAP (10 mg, 0.08 mmol) was added at 0° C. and reacted overnight at room temperature. After completion of the reaction, the mixture was diluted with DCM, washed with water, dried over anhydrous MgSO₄, filtered and concentrated. The concentrate was purified by silica gel column chromatography to obtain a yellow solid, (R)-2-(4-methylpiperazin-1-yl)-2-oxo-1-phenylethyl 3-amino-6-(1-(1-(tert-butoxycarbonyl)piperidin-4-yl)-1H-pyrazol-4-yl)pyrazine-2-carboxylate (68 mg, 0.11 mmol, 75%).

¹H NMR (400 MHz, CDCl₃) δ 8.42 (s, 1H), 7.92 (s, 1H), 7.90 (s, 1H), 7.62-7.55 (m, 2H), 7.49-7.39 (m, 3H), 6.66 (s, 1H), 6.49 (s, 2H), 4.36-4.20 (m, 3H), 3.80-3.61 (m, 2H), 3.61-3.53 (m, 1H), 3.50-3.39 (m, 1H), 2.97-2.85 (m, 2H), 2.51-2.41 (m, 1H), 2.36-2.27 (m, 2H), 2.24 (s, 3H), 2.20-2.11 (m, 2H), 2.01-1.87 (m, 3H), 1.50 (s, 9H).

### Synthesis of ((R)-2-(4-methylpiperazin-1-yl)-2-oxo-1-phenylethyl 3-amino-6-(1-(1-(tert-butoxycarbonyl)piperidin-4-yl)-1H-pyrazol-4-yl)pyrazine-2-carboxylate hydrochloride (TDH-455, step 3)

(R)-2-(4-methylpiperazin-1-yl)-2-oxo-1-phenylethyl 3-amino-6-(1-(1-(tert-butoxycarbonyl)piperidin-4-yl)-1H-pyrazol-4-yl)pyrazine-2-carboxylate (20 mg, 0.03 mmol) was dissolved in DCM (1.0 mL), 4 M HCl in dioxane (0.5 mL) was added, and reacted at room temperature for 2 hours. The reaction solvent was concentrated to yield a yellow solid, ((R)-2-(4-methylpiperazin-1-yl)-2-oxo-1-phenylethyl 3-amino-6-(1-(1-(tert-butoxycarbonyl)piperidin-4-yl)-1H-pyrazol-4-yl)pyrazine-2-carboxylate hydrochloride (1.7 mg, 0.003 mmol, 10%).

### Synthesis of (R)-2-((3-fluorophenyl)amino)-2-oxo-1-phenylethyl 3-amino-6-(1-(piperidin-4-yl)-1H-pyrazol-4-yl)pyrazine-2-carboxylate hydrochloride (TDH-456)

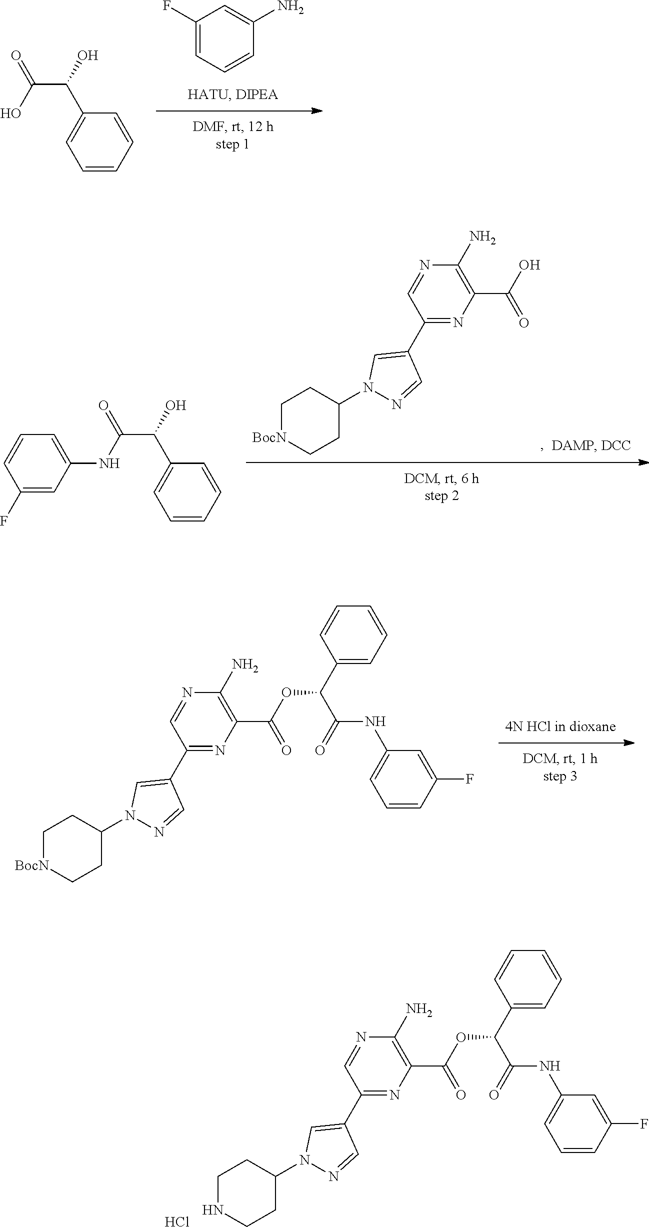

### Synthesis of (R)—N-(3-fluorophenyl)-2-hydroxy-2-phenylacetamide (step 1)

(R)-2-hydroxy-2-phenylacetic acid (200 mg, 1.3 mmol) was dissolved in DMF (5 mL), DIPEA (1.2 mL, 6.6 mmol) and HATU (748 mg, 2.0 mmol) were added thereto, and it was stirred for 20 minutes. 3-fluoroanilin (0.16 mL, 1.7 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired light orange solid, (R)—N-(3-fluorophenyl)-2-hydroxy-2-phenylacetamide (187 mg, 0.76 mmol, mixture).

### Synthesis of (R)-2-((3-fluorophenyl)amino)-2-oxo-1-phenylethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (50 mg, 0.13 mmol) was dissolved in DCM. Then, (R)—N-(3-fluorophenyl)-2-hydroxy-2-phenylacetamide (32 mg, 0.13 mmol), DCC (27 mg, 0.13 mmol), and DMAP (8 mg, 0.06 mmol) were added at 0° C., and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-((3-fluorophenyl)amino)-2-oxo-1-phenylethyl 3-amino-6-(1-(1-(tert-butoxycarbonyl)piperidin-4-yl)-1H-pyrazol-4-yl)pyrazine-2-carboxylate (80 mg, 0.13 mmol, quant.).

¹H NMR (400 MHz, CDCl₃) δ 9.04 (s, 1H), 8.55 (s, 1H), 8.24-8.19 (m, 0.5H), 8.01 (s, 1H), 7.96-7.91 (m, 1H), 7.68-7.63 (m, 2H), 7.53-7.40 (m, 4H), 7.27-7.20 (m, 1H), 7.18-7.09 (m, 1H), 6.53-6.49 (m, 0.5H), 6.45 (s, 1H), 6.35 (s, 2H), 4.40-4.24 (m, 3H), 2.99-2.86 (m, 2H), 2.25-2.13 (m, 2H), 1.98 (dd, J=12.3, 4.3 Hz, 2H).

### Synthesis of (R)-2-((3-fluorophenyl)amino)-2-oxo-1-phenylethyl 3-amino-6-(1-(piperidin-4-yl)-1H-pyrazol-4-yl)pyrazine-2-carboxylate hydrochloride (TDH-456, step 3)

(R)-2-((3-fluorophenyl)amino)-2-oxo-1-phenylethyl 3-amino-6-(1-(1-(tert-butoxycarbonyl)piperidin-4-yl)-1H-pyrazol-4-yl)pyrazine-2-carboxylate (20 mg, 0.03 mmol) was dissolved in DCM (1.0 mL), 4 M HCl in dioxane (0.5 mL) was added, and the mixture was reacted at room temperature for 2 hours. The reaction solvent was concentrated to obtain a yellow solid, (R)-2-((3-fluorophenyl)amino)-2-oxo-1-phenylethyl 3-amino-6-(1-(piperidin-4-yl)-1H-pyrazol-4-yl)pyrazine-2-carboxylate hydrochloride (16 mg, 0.03 mmol, 97%).

### Synthesis of (R)-2-((4-fluorobenzyl)amino)-2-oxo-1-phenylethyl 3-amino-6-(1-(piperidin-4-yl)-1H-pyrazol-4-yl)pyrazine-2-carboxylate hydrochloride (TDH-457)

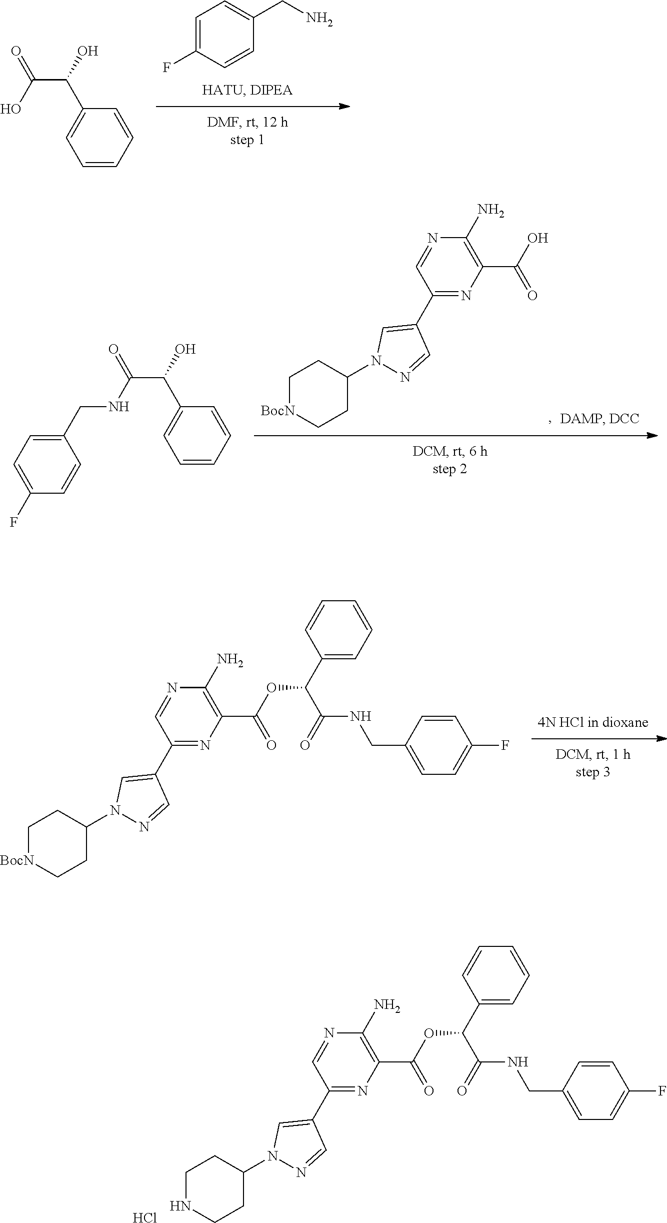

### Synthesis of (R)—N-(4-fluorobenzyl)-2-hydroxy-2-phenylacetamide (step 1)

(R)-2-hydroxy-2-phenylacetic acid (304 mg, 2.0 mmol) was dissolved in DMF (8 mL). Then, DIPEA (1.7 mL, 10 mmol) and HATU (1.14 g, 3.0 mmol) was added, and stirred for 20 minutes. 4-Fluorobenzylamine (0.30 mL, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired pale yellow solid, (R)—N-(4-fluorobenzyl)-2-hydroxy-2-phenylacetamide (527 mg, 2.0 mmol, quant.).

¹H NMR (300 MHz, CDCl₃) δ 7.46-7.33 (m, 5H), 7.21-7.13 (m, 2H), 7.04-6.96 (m, 2H), 6.64 (s, 1H), 5.10 (d, J=3.3 Hz, 1H), 4.42 (d, J=6.0 Hz, 2H), 3.82 (d, J=3.6 Hz, 1H).

### Synthesis of (R)-2-((4-fluorobenzyl)amino)-2-oxo-1-phenylethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)—N-(4-fluorobenzyl)-2-hydroxy-2-phenylacetamide (40 mg, 0.15 mmol), DCC (32 mg, 0.15 mmol), and DMAP (10 mg, 0.08 mmol) were added at 0° C., and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-((4-fluorobenzyl)amino)-2-oxo-1-phenylethyl 3-amino-6-(1-(1-(tert-butoxycarbonyl)piperidin-4-yl)-1H-pyrazol-4-yl)pyrazine-2-carboxylate (119 mg, 0.19 mmol, mixture).

### Synthesis of (R)-2-((4-fluorobenzyl)amino)-2-oxo-1-phenylethyl 3-amino-6-(1-(piperidin-4-yl)-1H-pyrazol-4-yl)pyrazine-2-carboxylate hydrochloride (TDH-457, step 3)

(R)-2-((4-fluorobenzyl)amino)-2-oxo-1-phenylethyl 3-amino-6-(1-(1-(tert-butoxycarbonyl)piperidin-4-yl)-1H-pyrazol-4-yl)pyrazine-2-carboxylate (20 mg, 0.03 mmol) was dissolved in DCM (1.0 mL), 4 M HCl in dioxane (0.5 mL) was added, and reacted at room temperature for 2 hours. The reaction solvent was concentrated to obtain a yellow solid, (R)-2-((4-fluorobenzyl)amino)-2-oxo-1-phenylethyl 3-amino-6-(1-(piperidin-4-yl)-1H-pyrazol-4-yl)pyrazine-2-carboxylate hydrochloride (8 mg, 0.01 mmol, 44%).

### Synthesis of (R)-2-((2-fluorophenyl)amino)-2-oxo-1-phenylethyl 3-amino-6-(1-(piperidin-4-yl)-1H-pyrazol-4-yl)pyrazine-2-carboxylate hydrochloride (TDH-458)

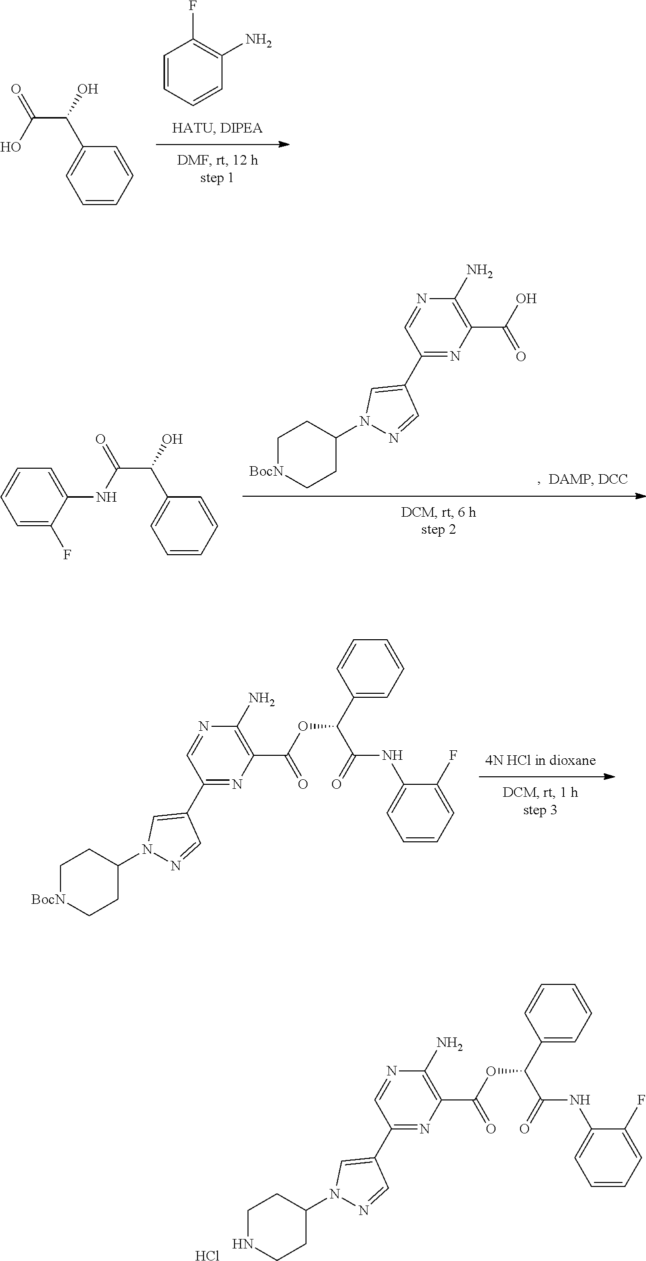

### Synthesis of (R)—N-(2-fluorophenyl)-2-hydroxy-2-phenylacetamide (step 1)

(R)-2-hydroxy-2-phenylacetic acid (304 mg, 2.0 mmol) was dissolved in DMF (8 mL). Then, DIPEA (1.7 mL, 10 mmol) and HATU (1.14 g, 3.0 mmol) were added, and stirred for 20 minutes. 2-fluoroanilin (0.25 mL, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired clear oil (R)—N-(2-fluorophenyl)-2-hydroxy-2-phenylacetamide (108 mg, 0.44 mmol, 22%).

¹H NMR (300 MHz, CDCl₃) δ 8.50 (s, 1H), 8.38-8.29 (m, 1H), 7.56-7.49 (m, 2H), 7.48-7.36 (m, 3H), 7.18-7.05 (m, 3H), 5.26 (d, J=3.4 Hz, 1H), 3.37 (d, J=3.4 Hz, 1H).

### Synthesis of (R)-2-((2-fluorophenyl)amino)-2-oxo-1-phenylethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)—N-(2-fluorophenyl)-2-hydroxy-2-phenylacetamide (38 mg, 0.15 mmol), DCC (32 mg, 0.15 mmol), and DMAP (10 mg, 0.08 mmol) were added at 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-((2-fluorophenyl)amino)-2-oxo-1-phenylethyl 3-amino-6-(1-(1-(tert-butoxycarbonyl)piperidin-4-yl)-1H-pyrazol-4-yl)pyrazine-2-carboxylate (96 mg, 0.16 mmol, mixture).

### Synthesis of (R)-2-((2-fluorophenyl)amino)-2-oxo-1-phenylethyl 3-amino-6-(1-(piperidin-4-yl)-1H-pyrazol-4-yl)pyrazine-2-carboxylate hydrochloride (TDH-458, step 3)

(R)-2-((2-fluorophenyl)amino)-2-oxo-1-phenylethyl 3-amino-6-(1-(1-(tert-butoxycarbonyl)piperidin-4-yl)-1H-pyrazol-4-yl)pyrazine-2-carboxylate (20 mg, 0.03 mmol) was dissolved in DCM (1.0 mL), 4 M HCl in dioxane (0.5 mL) was added, and reacted at room temperature for 2 hours. The reaction solvent was concentrated to yield a yellow solid, (R)-2-((2-fluorophenyl)amino)-2-oxo-1-phenylethyl 3-amino-6-(1-(piperidin-4-yl)-1H-pyrazol-4-yl)pyrazine-2-carboxylate hydrochloride (15 mg, 0.03 mmol, 85%).

### Synthesis of (R)-2-oxo-1-phenyl-2-((4-(trifluoromethyl)phenyl)amino)ethyl 3-amino-6-(1-(piperidin-4-yl)-1H-pyrazol-4-yl)pyrazine-2-carboxylate hydrochloride (TDH-459)

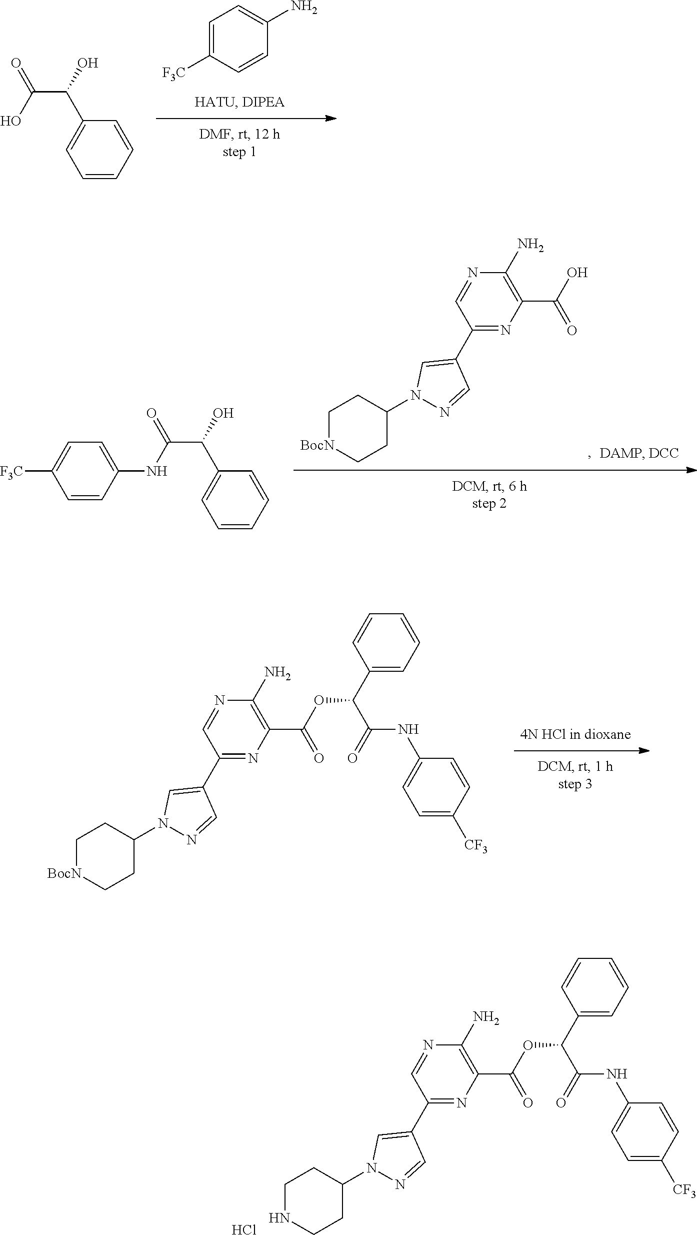

### Synthesis of (R)-2-hydroxy-2-phenyl-N-(4-(trifluoromethyl)phenyl)acetamide (step 1)

(R)-2-hydroxy-2-phenylacetic acid (304 mg, 2.0 mmol) was dissolved in DMF (8 mL). Then, DIPEA (1.7 mL, 6.6 mmol) and HATU (1.14 g, 3.0 mmol) were added, and stirred for 20 minutes. 4-(trifluoromethyl)aniline (0.33 mL, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvent was concentrated and then purified by column chromatography to obtain the desired light orange solid, (R)-2-hydroxy-2-phenyl-N-(4-(trifluoromethyl)phenyl)acetamide (105 mg, 0.35 mmol, 18%).

¹H NMR (300 MHz, CDCl₃) δ 8.47 (s, 1H), 7.70 (d, J=8.5 Hz, 2H), 7.59 (d, J=8.6 Hz, 2H), 7.54-7.47 (m, 2H), 7.47-7.38 (m, 3H), 5.25 (d, J=3.0 Hz, 1H), 3.29 (d, J=3.2 Hz, 1H).

### Synthesis of (R)-2-oxo-1-phenyl-2-((4-(trifluoromethyl)phenyl)amino)ethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)-2-hydroxy-2-phenyl-N-(4-(trifluoromethyl)phenyl)acetamide (46 mg, 0.15 mmol), DCC (32 mg, 0.15 mmol), and DMAP (10 mg, 0.08 mmol) were added at 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-oxo-1-phenyl-2-((4-(trifluoromethyl)phenyl)amino)ethyl 3-amino-6-(1-(1-(tert-butoxycarbonyl)piperidin-4-yl)-1H-pyrazol-4-yl)pyrazine-2-carboxylate (96 mg, 0.134 mmol, mixture).

### Synthesis of (R)-2-oxo-1-phenyl-2-((4-(trifluoromethyl)phenyl)amino)ethyl 3-amino-6-(1-(piperidin-4-yl)-1H-pyrazol-4-yl)pyrazine-2-carboxylate hydrochloride (TDH-459, step 3)

(R)-2-oxo-1-phenyl-2-((4-(trifluoromethyl)phenyl)amino)ethyl 3-amino-6-(1-(1-(tert-butoxycarbonyl)piperidin-4-yl)-1H-pyrazol-4-yl)pyrazine-2-carboxylate (20 mg, 0.03 mmol) was dissolved in DCM (1.0 mL), 4 M HCl in dioxane (0.5 mL) was added, and reacted at room temperature for 2 hours. The reaction solvent was concentrated to obtain a yellow solid, (R)-2-oxo-1-phenyl-2-((4-(trifluoromethyl)phenyl)amino)ethyl 3-amino-6-(1-(piperidin-4-yl)-1H-pyrazol-4-yl)pyrazine-2-carboxylate hydrochloride (19 mg, 0.03 mmol, quant.).

### Synthesis of (R)-2-oxo-1-phenyl-2-((3-(trifluoromethyl)phenyl)amino)ethyl 3-amino-6-(1-(piperidin-4-yl)-1H-pyrazol-4-yl)pyrazine-2-carboxylate hydrochloride (TDH-460)

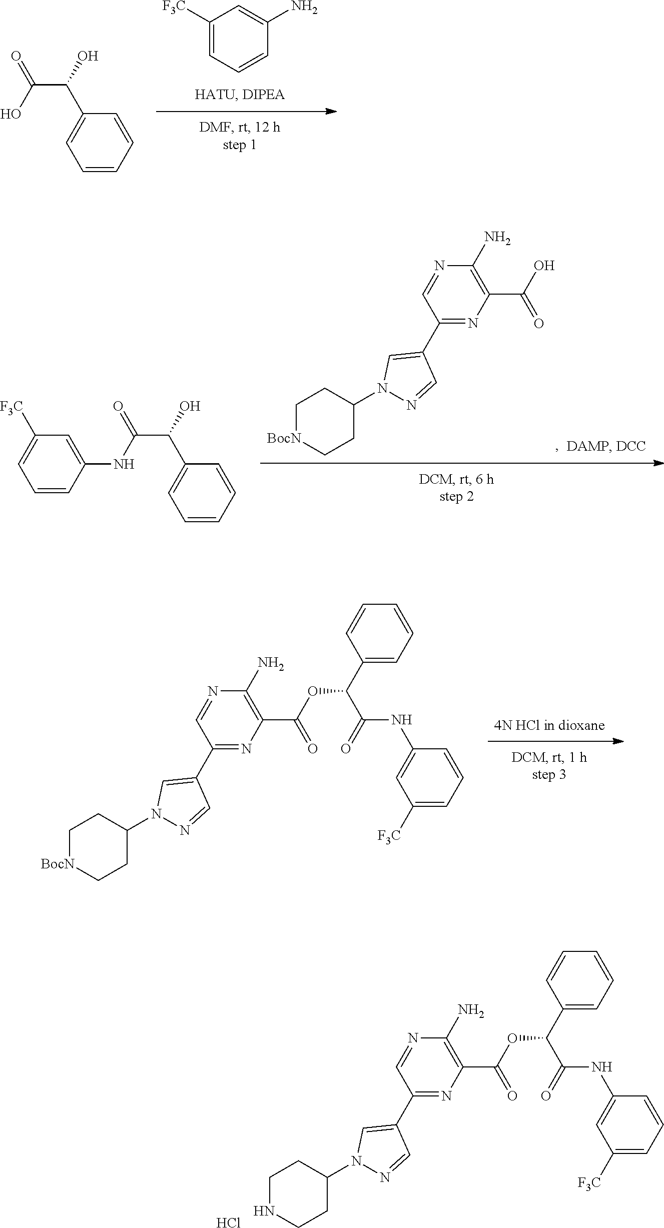

### Synthesis of (R)-2-hydroxy-2-phenyl-N-(3-(trifluoromethyl)phenyl)acetamide (step 1)

(R)-2-hydroxy-2-phenylacetic acid (304 mg, 2.0 mmol) was dissolved in DMF (8 mL). Then, DIPEA (1.7 mL, 6.6 mmol) and HATU (1.14 g, 3.0 mmol) were added, and stirred for 20 minutes. 3-(trifluoromethyl)aniline (0.33 mL, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired light orange solid, (R)-2-hydroxy-2-phenyl-N-(3-(trifluoromethyl)phenyl)acetamide (155 mg, 0.52 mmol, 26%).

¹H NMR (300 MHz, CDCl₃) δ 8.43 (s, 1H), 7.88 (s, 1H), 7.77 (d, J=7.8 Hz, 1H), 7.54-7.48 (m, 2H), 7.47-7.37 (m, 5H), 5.25 (d, J=3.0 Hz, 1H), 3.29 (s, 1H).

### Synthesis of (R)-2-oxo-1-phenyl-2-((3-(trifluoromethyl)phenyl)amino)ethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)-2-hydroxy-2-phenyl-N-(3-(trifluoromethyl)phenyl)acetamide (46 mg, 0.15 mmol), DCC (32 mg, 0.15 mmol), and DMAP (10 mg, 0.08 mmol) were added at 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-oxo-1-phenyl-2-((3-(trifluoromethyl)phenyl)amino)ethyl 3-amino-6-(1-(1-(tert-butoxycarbonyl)piperidin-4-yl)-1H-pyrazol-4-yl)pyrazine-2-carboxylate (94 mg, 0.14 mmol, mixture).

### Synthesis of (R)-2-oxo-1-phenyl-2-((3-(trifluoromethyl)phenyl)amino)ethyl 3-amino-6-(1-(piperidin-4-yl)-1H-pyrazol-4-yl)pyrazine-2-carboxylate hydrochloride (TDH-460, step 3)

(R)-2-oxo-1-phenyl-2-((3-(trifluoromethyl)phenyl)amino)ethyl 3-amino-6-(1-(1-(tert-butoxycarbonyl)piperidin-4-yl)-1H-pyrazol-4-yl)pyrazine-2-carboxylate (20 mg, 0.03 mmol) was dissolved in DCM (1.0 mL), 4 M HCl in dioxane (0.5 mL) was added, and reacted at room temperature for 2 hours. The reaction solvent was concentrated to obtain a yellow solid, (R)-2-oxo-1-phenyl-2-((3-(trifluoromethyl)phenyl)amino)ethyl 3-amino-6-(1-(piperidin-4-yl)-1H-pyrazol-4-yl)pyrazine-2-carboxylate hydrochloride (12 mg, 0.02 mmol, 66%).

### Synthesis of (R)-2-((4-(4-methylpiperazin-1-yl)phenyl)amino)-2-oxo-1-phenylethyl 3-amino-6-(1-(piperidin-4-yl)-1H-pyrazol-4-yl)pyrazine-2-carboxylate hydrochloride (TDH-461)

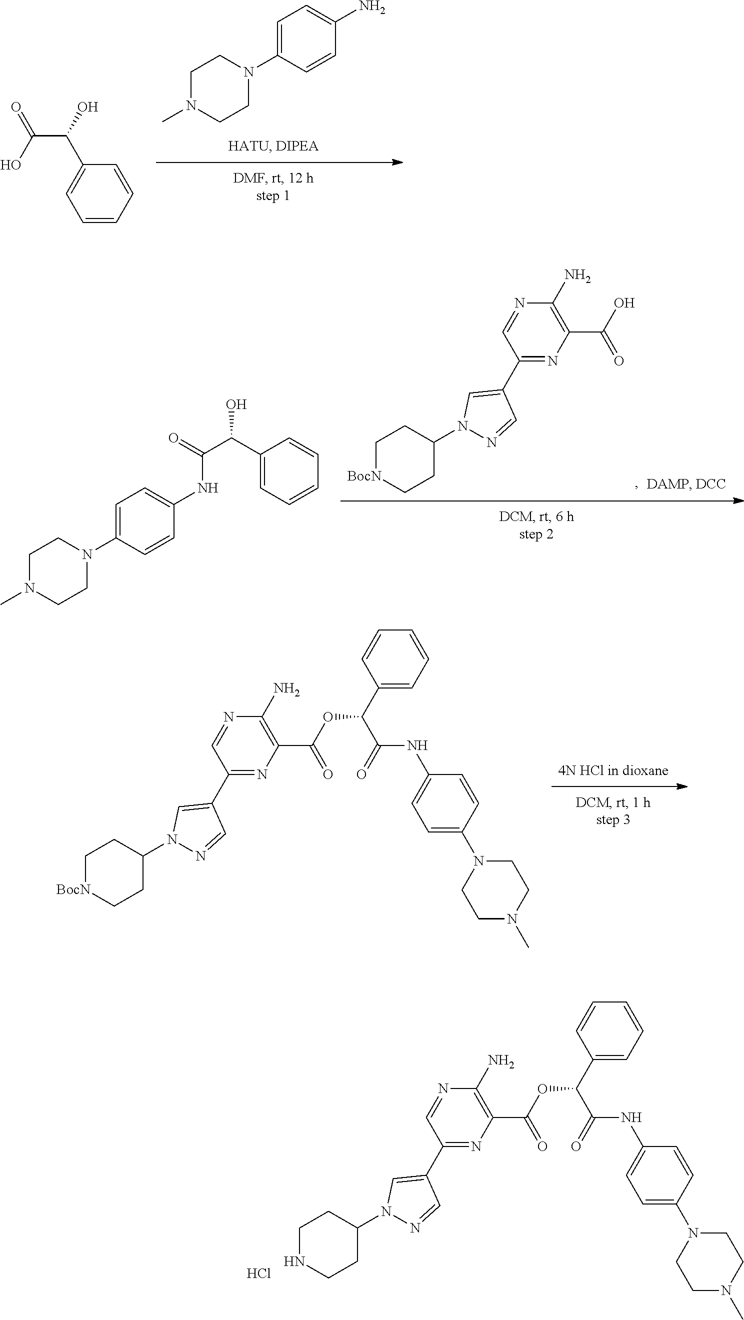

### Synthesis of (R)-2-hydroxy-N-(4-(4-methylpiperazin-1-yl)phenyl)-2-phenylacetamide (step 1)

(R)-2-hydroxy-2-phenylacetic acid (304 mg, 2.0 mmol) was dissolved in DMF (8 mL). Then, DIPEA (1.7 mL, 6.6 mmol) and HATU (1.14 g, 3.0 mmol) were added, and stirred for 20 minutes. 4-(4-methylpiperazin-1-yl)aniline (497 mg, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvent was concentrated and purified by column chromatography to obtain the desired dark orange solid, (R)-2-hydroxy-N-(4-(4-methylpiperazin-1-yl)phenyl)-2-phenylacetamide (297 mg, 0.91 mmol, 46%).

¹H NMR (300 MHz, CDCl₃) δ 7.93 (s, 1H), 7.53-7.49 (m, 2H), 7.46-7.38 (m, 5H), 6.94-6.86 (m, 2H), 5.21 (s, 1H), 3.22-3.13 (m, 4H), 2.64-2.56 (m, 4H), 2.37 (s, 3H).

### Synthesis of (R)-2-((4-(4-methylpiperazin-1-yl)phenyl)amino)-2-oxo-1-phenylethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)-2-hydroxy-N-(4-(4-methylpiperazin-1-yl)phenyl)-2-phenylacetamide (50 mg, 0.15 mmol), DCC (32 mg, 0.15 mmol), and DMAP (10 mg, 0.08 mmol) were added at 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-((4-(4-methylpiperazin-1-yl)phenyl)amino)-2-oxo-1-phenylethyl 3-amino-6-(1-(1-(tert-butoxycarbonyl)piperidin-4-yl)-1H-pyrazol-4-yl)pyrazine-2-carboxylate (80 mg, 0.12 mmol, 77%).

¹H NMR (400 MHz, CDCl₃) δ 8.79 (s, 1H), 8.52 (s, 1H), 8.02 (s, 1H), 7.91 (s, 1H), 7.69-7.63 (m, 2H), 7.50-7.40 (m, 5H), 6.89 (d, J=8.9 Hz, 2H), 6.45 (s, 1H), 6.35 (s, 2H), 4.37-4.23 (m, 3H), 3.19 (t, J=5.0 Hz, 4H), 2.97-2.85 (m, 2H), 2.59 (t, J=5.0 Hz, 4H), 2.37 (s, 3H), 2.02-1.92 (m, 2H), 1.50 (s, 9H).

### Synthesis of (R)-2-((4-(4-methylpiperazin-1-yl)phenyl)amino)-2-oxo-1-phenylethyl 3-amino-6-(1-(piperidin-4-yl)-1H-pyrazol-4-yl)pyrazine-2-carboxylate hydrochloride (TDH-461, step 3)

(R)-2-((4-(4-methylpiperazin-1-yl)phenyl)amino)-2-oxo-1-phenylethyl 3-amino-6-(1-(1-(tert-butoxycarbonyl)piperidin-4-yl)-1H-pyrazol-4-yl)pyrazine-2-carboxylate (20 mg, 0.03 mmol) was dissolved in DCM (1.0 mL), 4 M HCl in dioxane (0.5 mL) was added, and reacted at room temperature for 2 hours. The reaction solvent was concentrated to yield a yellow solid, (R)-2-((4-(4-methylpiperazin-1-yl)phenyl)amino)-2-oxo-1-phenylethyl 3-amino-6-(1-(piperidin-4-yl)-1H-pyrazol-4-yl)pyrazine-2-carboxylate hydrochloride (19 mg, 0.03 mmol, quant.).

### Synthesis of (R)-2-((3-(4-methylpiperazin-1-yl)phenyl)amino)-2-oxo-1-phenylethyl 3-amino-6-(1-(piperidin-4-yl)-1H-pyrazol-4-yl)pyrazine-2-carboxylate hydrochloride (TDH-462)

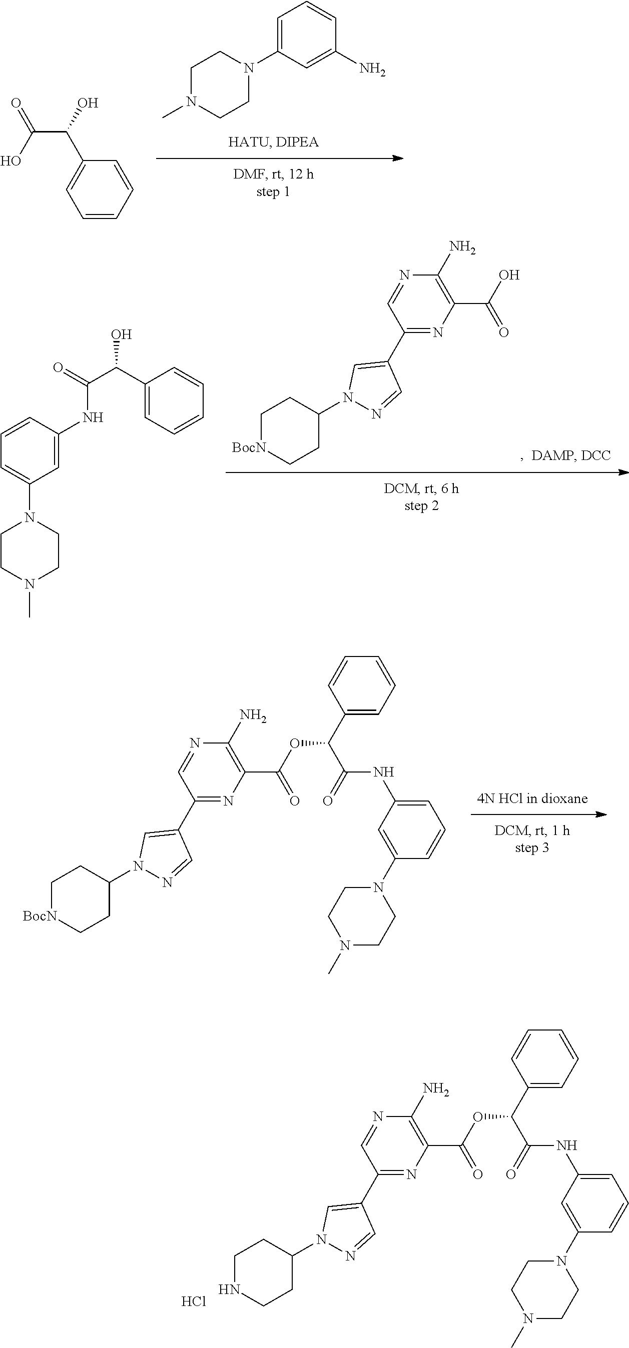

### Synthesis of (R)-2-hydroxy-N-(3-(4-methylpiperazin-1-yl)phenyl)-2-phenylacetamide (step 1)

(R)-2-hydroxy-2-phenylacetic acid (304 mg, 2.0 mmol) was dissolved in DMF (8 mL). Then, DIPEA (1.7 mL, 6.6 mmol) and HATU (1.14 g, 3.0 mmol) were added, and reacted at room temperature for 20 minutes. 3-(4-methylpiperazin-1-yl)aniline (497 mg, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired yellow solid, (R)-2-hydroxy-N-(3-(4-methylpiperazin-1-yl)phenyl)-2-phenylacetamide (638 mg, 1.96 mmol, 98%).

¹H NMR (300 MHz, CDCl₃) δ 8.08 (s, 1H), 7.55-7.48 (m, 2H), 7.47-7.37 (m, 4H), 7.25-7.16 (m, 1H), 6.89-6.83 (m, 1H), 6.74-6.68 (m, 1H), 5.22 (s, 1H), 3.30-3.20 (m, 4H), 2.67-2.57 (m, 4H), 2.38 (s, 3H).

### Synthesis of (R)-2-((3-(4-methylpiperazin-1-yl)phenyl)amino)-2-oxo-1-phenylethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)-2-hydroxy-N-(3-(4-methylpiperazin-1-yl)phenyl)-2-phenylacetamide (50 mg, 0.15 mmol), DCC (32 mg, 0.15 mmol), and DMAP (10 mg, 0.08 mmol) were added at 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-((3-(4-methylpiperazin-1-yl)phenyl)amino)-2-oxo-1-phenylethyl 3-amino-6-(1-(1-(tert-butoxycarbonyl)piperidin-4-yl)-1H-pyrazol-4-yl)pyrazine-2-carboxylate (71 mg, 0.10 mmol, 68%).

¹H NMR (400 MHz, CDCl₃) δ 8.89 (s, 1H), 8.53 (s, 1H), 8.02 (s, 1H), 7.93 (s, 1H), 7.69-7.62 (m, 2H), 7.51-7.47 (m, 1H), 7.47-7.37 (m, 3H), 7.21-7.14 (m, 1H), 6.85-6.80 (m, 1H), 6.73-6.67 (m, 1H), 6.42 (s, 1H), 6.34 (s, 2H), 4.36-4.20 (m, 3H), 3.25-3.15 (m, 4H), 2.98-2.86 (m, 2H), 2.59-2.51 (m, 4H), 2.36 (s, 3H), 2.23-2.14 (m, 2H), 2.01-1.92 (m, 2H), 1.50 (s, 9H).

### Synthesis of (R)-2-((3-(4-methylpiperazin-1-yl)phenyl)amino)-2-oxo-1-phenylethyl 3-amino-6-(1-(piperidin-4-yl)-1H-pyrazol-4-yl)pyrazine-2-carboxylate hydrochloride (TDH-462, step 3)

(R)-2-((3-(4-methylpiperazin-1-yl)phenyl)amino)-2-oxo-1-phenylethyl 3-amino-6-(1-(1-(tert-butoxycarbonyl)piperidin-4-yl)-1H-pyrazol-4-yl)pyrazine-2-carboxylate (20 mg, 0.03 mmol) was dissolved in DCM (1.0 mL), 4 M HCl in dioxane (0.5 mL) was added, and it was reacted at room temperature for 2 hours. The reaction solvent was concentrated to yield a yellow solid, (R)-2-((3-(4-methylpiperazin-1-yl)phenyl)amino)-2-oxo-1-phenylethyl 3-amino-6-(1-(piperidin-4-yl)-1H-pyrazol-4-yl)pyrazine-2-carboxylate hydrochloride (18 mg, 0.03 mmol, quant.).

### Synthesis of (R)-2-oxo-1-phenyl-2-(phenylamino)ethyl 3-amino-6-(1-(piperidin-4-yl)-1H-pyrazol-4-yl)pyrazine-2-carboxylate hydrochloride (TDH-463)

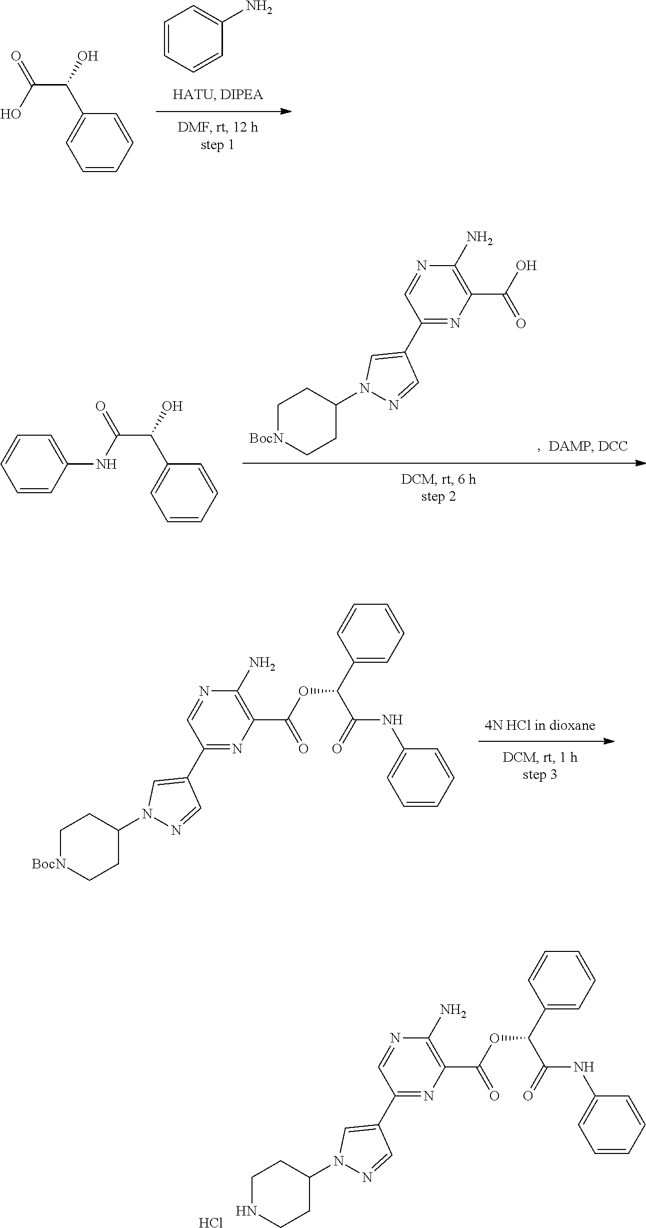

### Synthesis of (R)-2-hydroxy-N,2-diphenylacetamide (step 1)

(R)-2-hydroxy-2-phenylacetic acid (304 mg, 2.0 mmol) was dissolved in DMF (8 mL). Then, DIPEA (1.7 mL, 6.6 mmol) and HATU (1.14 g, 3.0 mmol) were added, and stirred for 20 minutes. Aniline (0.24 mL, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and then purified by column chromatography to obtain the desired yellow oil (R)-2-hydroxy-N,2-diphenylacetamide (386 mg, 1.70 mmol, 85%).

¹H NMR (400 MHz, CDCl₃) b 9.01 (s, 1H), 7.59-7.50 (m, 2H), 7.50-7.41 (m, 2H), 7.41-7.29 (m, 5H), 7.21-7.12 (m, 1H), 5.54 (s, 1H), 5.07 (s, 1H).

### Synthesis of (R)-2-oxo-1-phenyl-2-(phenylamino)ethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)-2-hydroxy-N,2-diphenylacetamide (35 mg, 0.15 mmol), DCC (32 mg, 0.15 mmol), and DMAP (10 mg, 0.08 mmol) were added at 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-oxo-1-phenyl-2-(phenylamino)ethyl 3-amino-6-(1-(1-(tert-butoxycarbonyl)piperidin-4-yl)-1H-pyrazol-4-yl)pyrazine-2-carboxylate (101 mg, 0.17 mmol, mixture).

### Synthesis of (R)-2-oxo-1-phenyl-2-(phenylamino)ethyl 3-amino-6-(1-(piperidin-4-yl)-1H-pyrazol-4-yl)pyrazine-2-carboxylate hydrochloride (TDH-463, step 3)

(R)-2-oxo-1-phenyl-2-(phenylamino)ethyl 3-amino-6-(1-(1-(tert-butoxycarbonyl)piperidin-4-yl)-1H-pyrazol-4-yl)pyrazine-2-carboxylate (25 mg, 0.04 mmol) was dissolved in DCM (1.0 mL), 4 M HCl in dioxane (0.5 mL) was added, and it was reacted at room temperature for 2 hours. The reaction solvent was concentrated to obtain a yellow solid, (R)-2-oxo-1-phenyl-2-(phenylamino)ethyl 3-amino-6-(1-(piperidin-4-yl)-1H-pyrazol-4-yl)pyrazine-2-carboxylate hydrochloride (12 mg, 0.022 mmol, 54%).

### Synthesis of (R)-2-((4-fluoro-3-methylphenyl)amino)-2-oxo-1-phenylethyl 3-amino-6-(1-(piperidin-4-yl)-1H-pyrazol-4-yl)pyrazine-2-carboxylate hydrochloride (TDH-464)

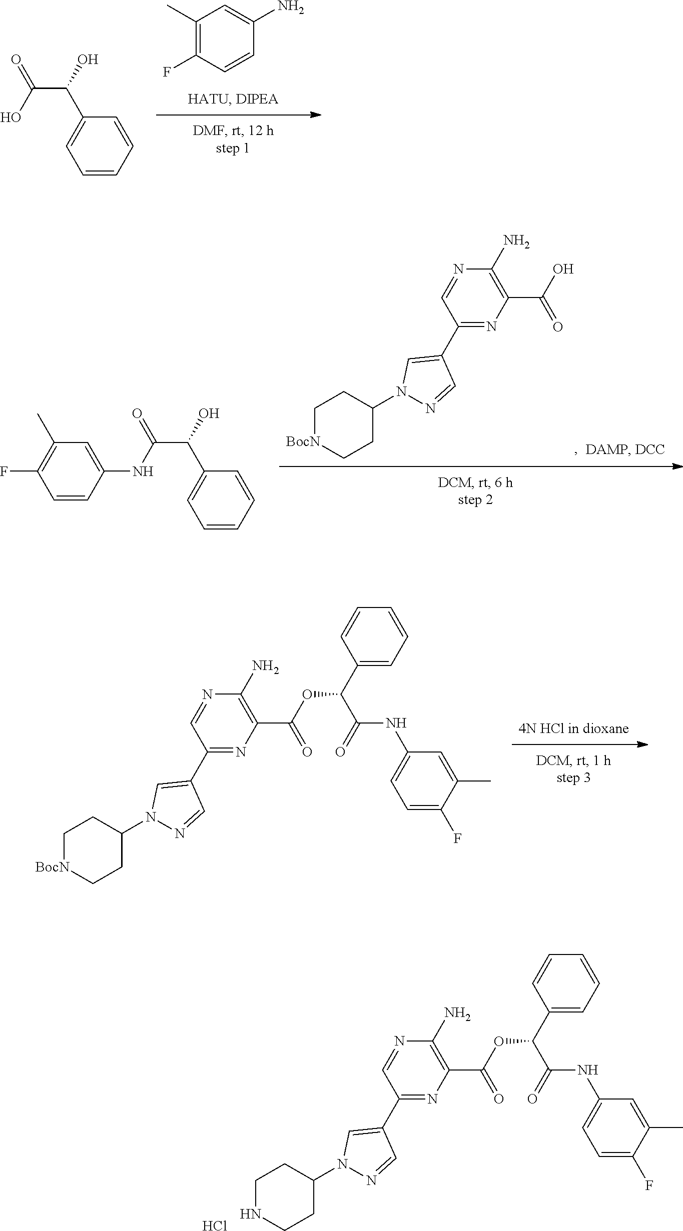

### Synthesis of (R)—N-(4-fluoro-3-methylphenyl)-2-hydroxy-2-phenylacetamide (step 1)

(R)-2-hydroxy-2-phenylacetic acid (304 mg, 2.0 mmol) was dissolved in DMF (8 mL). Then, DIPEA (1.7 mL, 6.6 mmol) and HATU (1.14 g, 3.0 mmol) were added, and stirred for 20 minutes. 4-fluoro-3-methylaniline (325 mg, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and then purified by column chromatography to obtain the desired white solid, (R)—N-(4-fluoro-3-methylphenyl)-2-hydroxy-2-phenylacetamide (362 mg, 1.40 mmol, 70%).

¹H NMR (500 MHz, CDCl₃) δ 8.98 (s, 1H), 7.47-7.38 (m, 2H), 7.36-7.28 (m, 4H), 7.27-7.19 (m, 1H), 6.96-6.85 (m, 1H), 5.74 (s, 1H), 5.05 (s, 1H), 3.29 (s, 3H).

### Synthesis of (R)-2-((4-fluoro-3-methylphenyl)amino)-2-oxo-1-phenylethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)—N-(4-fluoro-3-methylphenyl)-2-hydroxy-2-phenylacetamide (39 mg, 0.15 mmol), DCC (32 mg, 0.15 mmol), and DMAP (10 mg, 0.08 mmol) were added at 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-((4-fluoro-3-methylphenyl)amino)-2-oxo-1-phenylethyl 3-amino-6-(1-(1-(tert-butoxycarbonyl)piperidin-4-yl)-1H-pyrazol-4-yl)pyrazine-2-carboxylate (77 mg, 0.12 mmol, 82%).

¹H NMR (400 MHz, CDCl₃) b 8.91 (s, 1H), 8.53 (s, 1H), 8.02 (s, 1H), 7.90 (s, 1H), 7.74-7.64 (m, 2H), 7.50-7.40 (m, 4H), 7.34-7.29 (m, 1H), 7.00-6.90 (m, 1H), 6.44 (s, 1H), 6.35 (s, 2H), 4.40-4.21 (m, 3H), 3.02-2.86 (m, 2H), 2.23 (s, 3H), 2.21-2.13 (m, 2H), 2.03-1.88 (m, 3H), 1.50 (s, 9H).

### Synthesis of (R)-2-((4-fluoro-3-methylphenyl)amino)-2-oxo-1-phenylethyl 3-amino-6-(1-(piperidin-4-yl)-1H-pyrazol-4-yl)pyrazine-2-carboxylate hydrochloride (TDH-464, step 3)

(R)-2-((4-fluoro-3-methylphenyl)amino)-2-oxo-1-phenylethyl 3-amino-6-(1-(1-(tert-butoxycarbonyl)piperidin-4-yl)-1H-pyrazol-4-yl)pyrazine-2-carboxylate (25 mg, 0.04 mmol) was dissolved in DCM (1.0 mL), 4 M HCl in dioxane (0.5 mL) was added, and it was reacted at room temperature for 2 hours. The reaction solvent was concentrated to yield a yellow solid, (R)-2-((4-fluoro-3-methylphenyl)amino)-2-oxo-1-phenylethyl 3-amino-6-(1-(piperidin-4-yl)-1H-pyrazol-4-yl)pyrazine-2-carboxylate hydrochloride (18 mg, 0.03 mmol, 81%).

### Synthesis of (R)-1-(2-chlorophenyl)-2-((4-fluorophenyl)amino)-2-oxoethyl 3-amino-6-(1-(piperidin-4-yl)-1H-pyrazol-4-yl)pyrazine-2-carboxylate hydrochloride (TDH-465)

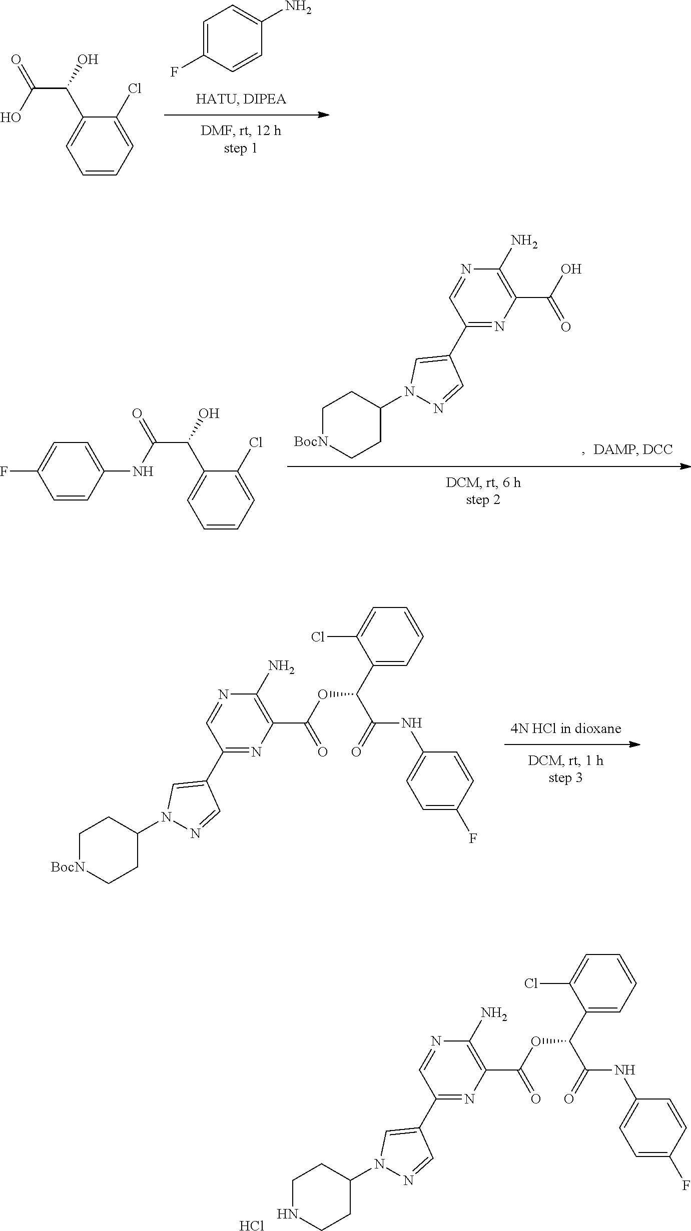

### Synthesis of (R)-2-(2-chlorophenyl)-N-(4-fluorophenyl)-2-hydroxyacetamide (step 1)

(R)-2-(2-chlorophenyl)-2-hydroxyacetic acid (373 mg, 2.0 mmol) was dissolved in DMF (8 mL). Then, and DIPEA (1.7 mL, 6.6 mmol) and HATU (1.14 g, 3.0 mmol) were added, and stirred for 20 minutes. 4-fluoroaniline (0.3 mL, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired white solid, (R)-2-(2-chlorophenyl)-N-(4-fluorophenyl)-2-hydroxyacetamide (239 mg, 0.86 mmol, 43%).

¹H NMR (400 MHz, DMSO) δ 10.13 (s, 1H), 7.80-7.70 (m, 2H), 7.59-7.52 (m, 1H), 7.52-7.42 (m, 1H), 7.42-7.30 (m, 2H), 7.23-7.07 (m, 2H), 6.67 (s, 1H), 5.47 (s, 1H).

### Synthesis of (R)-1-(2-chlorophenyl)-2-((4-fluorophenyl)amino)-2-oxoethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)-2-(2-chlorophenyl)-N-(4-fluorophenyl)-2-hydroxyacetamide (42 mg, 0.15 mmol), DCC (32 mg, 0.15 mmol), and DMAP (10 mg, 0.08 mmol) were added at 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-1-(2-chlorophenyl)-2-((4-fluorophenyl)amino)-2-oxoethyl 3-amino-6-(1-(1-(tert-butoxycarbonyl)piperidin-4-yl)-1H-pyrazol-4-yl)pyrazine-2-carboxylate (102 mg, 0.16 mmol, mixture).

### Synthesis of (R)-1-(2-chlorophenyl)-2-((4-fluorophenyl)amino)-2-oxoethyl 3-amino-6-(1-(piperidin-4-yl)-1H-pyrazol-4-yl)pyrazine-2-carboxylate hydrochloride (TDH-465, step 3)

(R)-1-(2-chlorophenyl)-2-((4-fluorophenyl)amino)-2-oxoethyl 3-amino-6-(1-(1-(tert-butoxycarbonyl)piperidin-4-yl)-1H-pyrazol-4-yl)pyrazine-2-carboxylate (25 mg, 0.04 mmol) was dissolved in DCM (1.0 mL), 4 M HCl in dioxane (0.5 mL) was added, and it was reacted at room temperature for 2 hours. The reaction solvent was concentrated to obtain a yellow solid, (R)-1-(2-chlorophenyl)-2-((4-fluorophenyl)amino)-2-oxoethyl 3-amino-6-(1-(piperidin-4-yl)-1H-pyrazol-4-yl)pyrazine-2-carboxylate hydrochloride (21 mg, 0.036 mmol, 94%).

### Synthesis of (R)-2-oxo-1-phenyl-2-(pyridin-3-ylamino)ethyl 3-amino-6-(1-(piperidin-4-yl)-1H-pyrazol-4-yl)pyrazine-2-carboxylate hydrochloride (TDH-468)

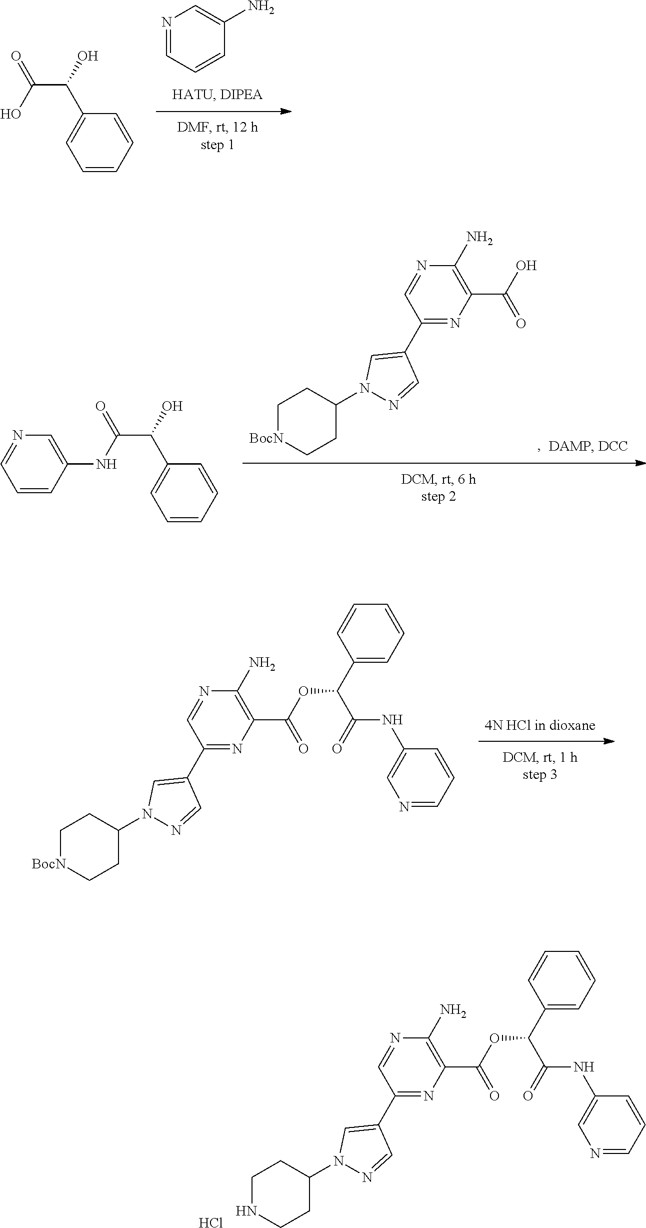

### Synthesis of (R)-2-hydroxy-2-phenyl-N-(pyridin-3-yl)acetamide (step 1)

(R)-2-hydroxy-2-phenylacetic acid (304 mg, 2.0 mmol) was dissolved in DMF (8 mL). Then, DIPEA (1.7 mL, 6.6 mmol) and HATU (1.14 g, 3.0 mmol) were added, and stirred for 20 minutes. Pyridin-3-amine (244 mg, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired white solid, (R)-2-hydroxy-2-phenyl-N-(pyridin-3-yl)acetamide (174 mg, 0.76 mmol, 38%).

¹H NMR (400 MHz, DMSO) δ 10.20 (s, 1H), 8.90-8.87 (m, 1H), 8.30-8.24 (m, 1H), 8.16-8.10 (m, 1H), 7.56-7.50 (m, 2H), 7.40-7.29 (m, 4H), 6.55 (s, 1H), 5.14 (s, 1H).

### Synthesis of (R)-2-oxo-1-phenyl-2-(pyridin-3-ylamino)ethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)-2-hydroxy-2-phenyl-N-(pyridin-3-yl)acetamide (34 mg, 0.15 mmol), DCC (32 mg, 0.15 mmol), and DMAP (10 mg, 0.08 mmol) were added to 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-oxo-1-phenyl-2-(pyridin-3-ylamino)ethyl 3-amino-6-(1-(1-(tert-butoxycarbonyl)piperidin-4-yl)-1H-pyrazol-4-yl)pyrazine-2-carboxylate (110 mg, 0.18 mmol, mixture).

### Synthesis of (R)-2-oxo-1-phenyl-2-(pyridin-3-ylamino)ethyl 3-amino-6-(1-(piperidin-4-yl)-1H-pyrazol-4-yl)pyrazine-2-carboxylate hydrochloride (TDH-468, step 3)

(R)-2-oxo-1-phenyl-2-(pyridin-3-ylamino)ethyl 3-amino-6-(1-(1-(tert-butoxycarbonyl)piperidin-4-yl)-1H-pyrazol-4-yl)pyrazine-2-carboxylate (30 mg, 0.05 mmol) was dissolved in DCM (1.0 mL), 4 M HCl in dioxane (0.5 mL) was added, and it was reacted at room temperature for 2 hours. The reaction solvent was concentrated to obtain a yellow solid, (R)-2-oxo-1-phenyl-2-(pyridin-3-ylamino)ethyl 3-amino-6-(1-(piperidin-4-yl)-1H-pyrazol-4-yl)pyrazine-2-carboxylate hydrochloride (11 mg, 0.02 mmol, 41%).

### Synthesis of (R)-2-((3-(dimethylamino)phenyl)amino)-2-oxo-1-phenylethyl 3-amino-6-(1-(piperidin-4-yl)-1H-pyrazol-4-yl)pyrazine-2-carboxylate hydrochloride (TDH-469)

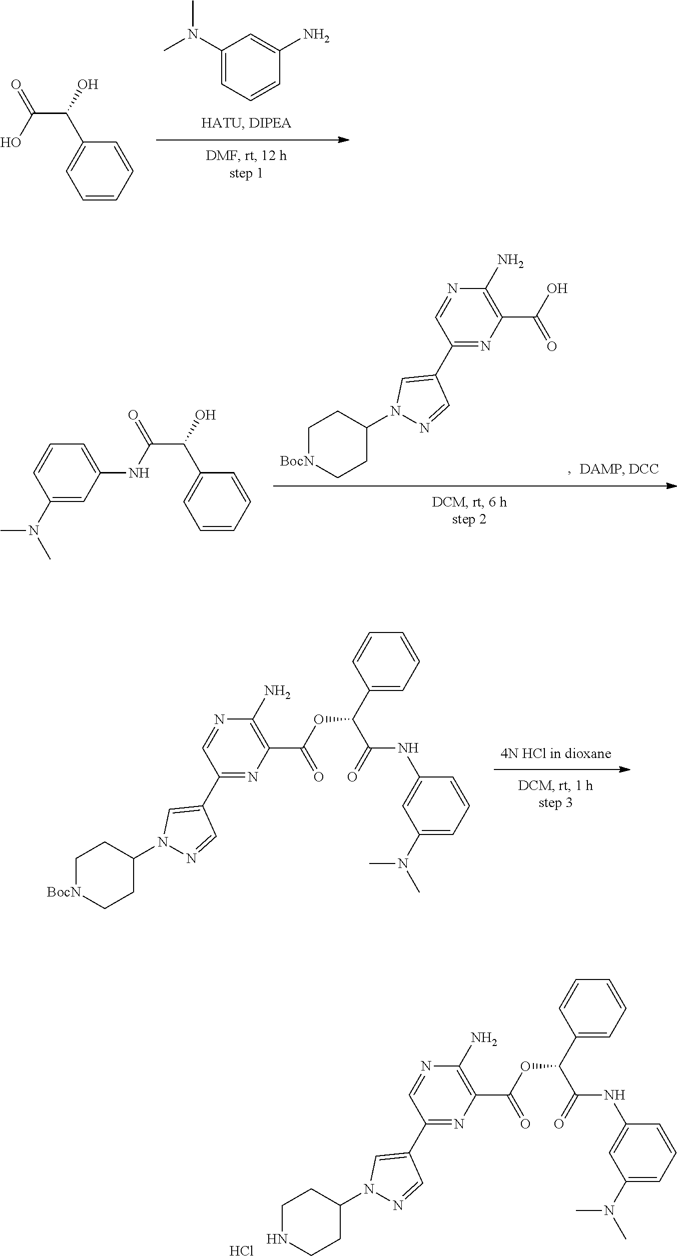

### Synthesis of (R)—N-(3-(dimethylamino)phenyl)-2-hydroxy-2-phenylacetamide (step 1)

(R)-2-hydroxy-2-phenylacetic acid (304 mg, 2.0 mmol) was dissolved in DMF (8 mL). Then, DIPEA (1.7 mL, 6.6 mmol) and HATU (1.14 g, 3.0 mmol) were added, and stirred for 20 minutes. N¹,N¹-dimethylbenzene-1,3-diamine (354 mg, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and then purified by column chromatography to obtain the desired pale yellow solid, (R)—N-(3-(dimethylamino)phenyl)-2-hydroxy-2-phenylacetamide (507 mg, 1.88 mmol, 96%).

¹H NMR (300 MHz, DMSO) δ 9.67 (s, 1H), 7.56-7.48 (m, 2H), 7.42-7.28 (m, 3H), 7.16-7.11 (m, 1H), 7.11-7.02 (m, 2H), 6.50-6.35 (m, 2H), 5.08 (s, 1H), 2.86 (s, 6H).

### Synthesis of (R)-2-((3-(dimethylamino)phenyl)amino)-2-oxo-1-phenylethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)—N-(3-(dimethylamino)phenyl)-2-hydroxy-2-phenylacetamide (41 mg, 0.15 mmol), DCC (32 mg, 0.15 mmol), and DMAP (10 mg, 0.08 mmol) were added to 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-((3-(dimethylamino)phenyl)amino)-2-oxo-1-phenylethyl 3-amino-6-(1-(1-(tert-butoxycarbonyl)piperidin-4-yl)-1H-pyrazol-4-yl)pyrazine-2-carboxylate (127 mg, 0.20 mmol, mixture).

### Synthesis of (R)-2-((3-(dimethylamino)phenyl)amino)-2-oxo-1-phenylethyl 3-amino-6-(1-(piperidin-4-yl)-1H-pyrazol-4-yl)pyrazine-2-carboxylate hydrochloride (TDH-469, step 3)

(R)-2-((3-(dimethylamino)phenyl)amino)-2-oxo-1-phenylethyl 3-amino-6-(1-(1-(tert-butoxycarbonyl)piperidin-4-yl)-1H-pyrazol-4-yl)pyrazine-2-carboxylate (50 mg, 0.08 mmol) was dissolved in DCM (1.0 mL), 4 M HCl in dioxane (0.5 mL) was added, and it was reacted at room temperature for 2 hours. The reaction solvent was concentrated to yield a yellow solid, (R)-2-((3-(dimethylamino)phenyl)amino)-2-oxo-1-phenylethyl 3-amino-6-(1-(piperidin-4-yl)-1H-pyrazol-4-yl)pyrazine-2-carboxylate hydrochloride (12 mg, 0.02 mmol, 26%).

### Synthesis of (R)-2-((4-(dimethylamino)phenyl)amino)-2-oxo-1-phenylethyl 3-amino-6-(1-(piperidin-4-yl)-1H-pyrazol-4-yl)pyrazine-2-carboxylate hydrochloride (TDH-470)

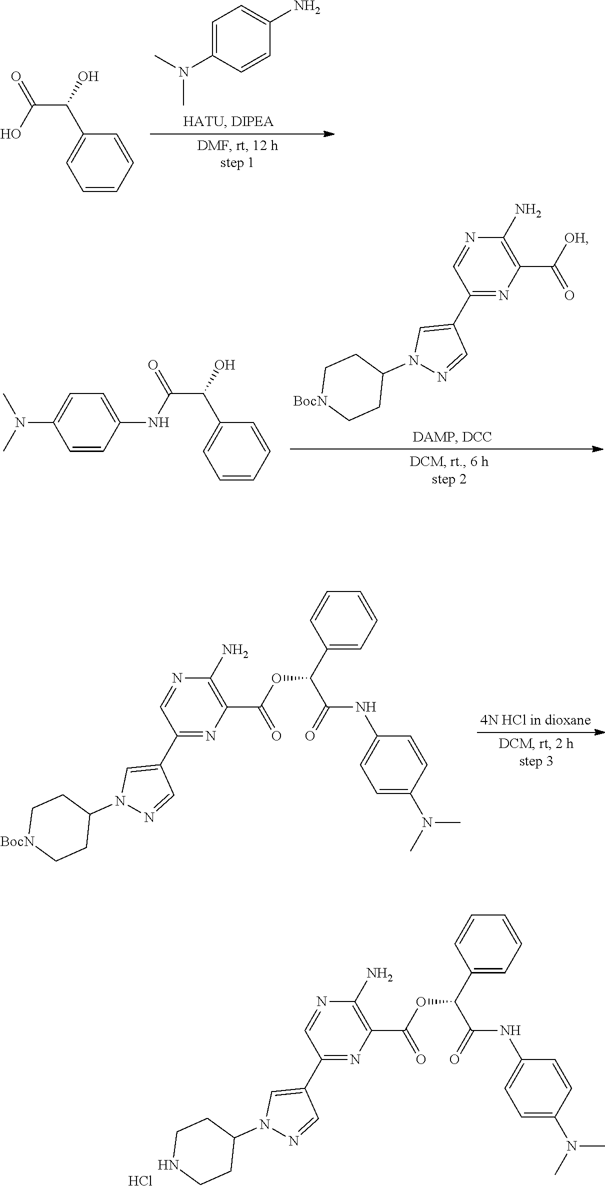

### Synthesis of (R)—N-(4-(dimethylamino)phenyl)-2-hydroxy-2-phenylacetamide (step 1)

(R)-2-hydroxy-2-phenylacetic acid (304 mg, 2.0 mmol) was dissolved in DMF (8 mL). Then, DIPEA (1.7 mL, 6.6 mmol) and HATU (1.14 g, 3.0 mmol) were added, and stirred for 20 minutes. N¹,N¹-dimethylbenzene-1,4-diamine (354 mg, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired pale yellow solid, (R)—N-(4-(dimethylamino)phenyl)-2-hydroxy-2-phenylacetamide (507 mg, 1.88 mmol, 94%).

¹H NMR (300 MHz, DMSO) δ 9.62 (s, 1H), 7.56-7.45 (m, 4H), 7.39-7.25 (m, 3H), 6.72-6.62 (m, 2H), 6.33 (s, 1H), 5.05 (s, 1H), 2.84 (s, 6H).

### Synthesis of (R)-2-((4-(dimethylamino)phenyl)amino)-2-oxo-1-phenylethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)—N-(4-(dimethylamino)phenyl)-2-hydroxy-2-phenylacetamide (41 mg, 0.15 mmol), DCC (32 mg, 0.15 mmol), and DMAP (10 mg, 0.08 mmol) were added to 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-((4-(dimethylamino)phenyl)amino)-2-oxo-1-phenylethyl 3-amino-6-(1-(1-(tert-butoxycarbonyl)piperidin-4-yl)-1H-pyrazol-4-yl)pyrazine-2-carboxylate (94 mg, 0.15 mmol, mixture).

### Synthesis of (R)-2-((4-(dimethylamino)phenyl)amino)-2-oxo-1-phenylethyl 3-amino-6-(1-(piperidin-4-yl)-1H-pyrazol-4-yl)pyrazine-2-carboxylate hydrochloride (TDH-470, step 3)

(R)-2-((4-(dimethylamino)phenyl)amino)-2-oxo-1-phenylethyl 3-amino-6-(1-(1-(tert-butoxycarbonyl)piperidin-4-yl)-1H-pyrazol-4-yl)pyrazine-2-carboxylate (50 mg, 0.08 mmol) was dissolved in DCM (1.0 mL), 4 M HCl in dioxane (0.5 mL) was added, and it was reacted at room temperature for 2 hours. The reaction solvent was concentrated to yield a yellow solid, (R)-2-((4-(dimethylamino)phenyl)amino)-2-oxo-1-phenylethyl 3-amino-6-(1-(piperidin-4-yl)-1H-pyrazol-4-yl)pyrazine-2-carboxylate hydrochloride (12 mg, 0.02 mmol, 26%).

### Synthesis of (R)-1-((4-fluorophenyl)amino)-3-methyl-1-oxobutan-2-yl 3-amino-6-(1-(piperidin-4-yl)-1H-pyrazol-4-yl)pyrazine-2-carboxylate hydrochloride (TDH-471)

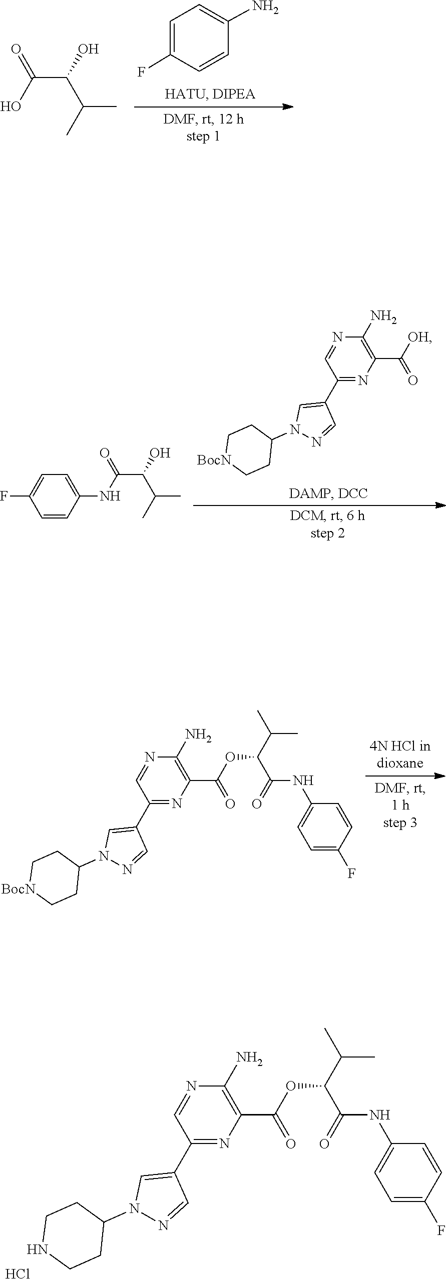

### Synthesis of (R)—N-(4-fluorophenyl)-2-hydroxy-3-methylbutanamide (step 1)

(R)-2-hydroxy-3-methylbutanoic acid (373 mg, 2.0 mmol) was dissolved in DMF (8 mL). Then, DIPEA (1.7 mL, 6.6 mmol) and HATU (1.14 g, 3.0 mmol) were added, and stirred for 20 minutes. 4-fluoroaniline (0.3 mL, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired yellow oil (R)—N-(4-fluorophenyl)-2-hydroxy-3-methylbutanamide (209 mg, 0.99 mmol, 50%).

¹H NMR (300 MHz, DMSO) δ 9.71 (s, 1H), 7.78-7.68 (m, 2H), 7.21-7.10 (m, 2H), 5.68 (d, J=4.9 Hz, 1H), 3.81 (s, 1H), 2.12-2.02 (m, 1H), 0.95 (d, J=6.9 Hz, 3H), 0.85 (d, J=6.8 Hz, 3H).

### Synthesis of (R)-1-((4-fluorophenyl)amino)-3-methyl-1-oxobutan-2-yl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)—N-(4-fluorophenyl)-2-hydroxy-3-methylbutanamide (32 mg, 0.15 mmol), DCC (32 mg, 0.15 mmol), and DMAP (10 mg, 0.08 mmol) were added at 0° C., and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-1-((4-fluorophenyl)amino)-3-methyl-1-oxobutan-2-yl 3-amino-6-(1-(1-(tert-butoxycarbonyl)piperidin-4-yl)-1H-pyrazol-4-yl)pyrazine-2-carboxylate (131 mg, 0.23 mmol, mixture).

### Synthesis of (R)-1-((4-fluorophenyl)amino)-3-methyl-1-oxobutan-2-yl 3-amino-6-(1-(piperidin-4-yl)-1H-pyrazol-4-yl)pyrazine-2-carboxylate hydrochloride (TDH-471, step 3)

(R)-1-((4-fluorophenyl)amino)-3-methyl-1-oxobutan-2-yl 3-amino-6-(1-(1-(tert-butoxycarbonyl)piperidin-4-yl)-1H-pyrazol-4-yl)pyrazine-2-carboxylate (60 mg, 0.10 mmol) was dissolved in DCM (2.0 mL), 4 M HCl in dioxane (1.0 mL) was added, and it was reacted at room temperature for 2 hours. The reaction solvent was concentrated to obtain a yellow solid, (R)-1-((4-fluorophenyl)amino)-3-methyl-1-oxobutan-2-yl 3-amino-6-(1-(piperidin-4-yl)-1H-pyrazol-4-yl)pyrazine-2-carboxylate hydrochloride (18 mg, 0.035 mmol, 35%).

### Synthesis of (R)-2-oxo-1-phenyl-2-(pyridin-4-ylamino)ethyl 3-amino-6-(1-(piperidin-4-yl)-1H-pyrazol-4-yl)pyrazine-2-carboxylate 2,2,2-trifluoroacetate (TDH-479)

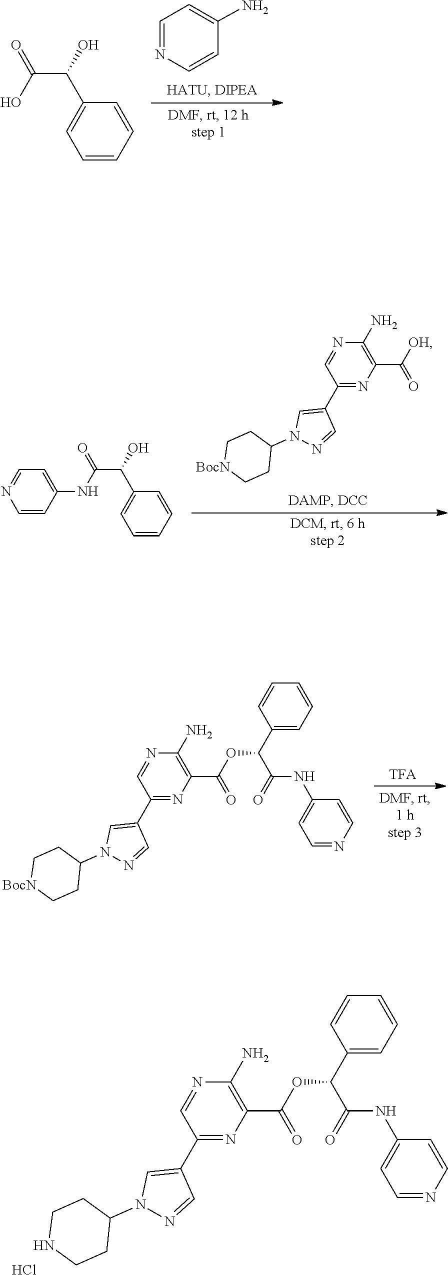

### Synthesis of (R)-2-hydroxy-2-phenyl-N-(pyridin-4-yl)acetamide (step 1)

(R)-2-hydroxy-2-phenylacetic acid (304 mg, 2.0 mmol) was dissolved in DMF (8 mL). Then, DIPEA (1.7 mL, 6.6 mmol) and HATU (1.14 g, 3.0 mmol) were added, and stirred for 20 minutes. Pyridin-4-amine (244 mg, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired yellow oil (R)-2-hydroxy-2-phenyl-N-(pyridin-4-yl)acetamide (209 mg, 0.99 mmol, 50%).

¹H NMR (300 MHz, DMSO) δ 9.71 (s, 1H), 7.78-7.68 (m, 2H), 7.21-7.10 (m, 2H), 5.68 (d, J=4.9 Hz, 1H), 3.81 (s, 1H), 2.12-2.02 (m, 1H), 0.95 (d, J=6.9 Hz, 3H), 0.85 (d, J=6.8 Hz, 3H).

### Synthesis of (R)-2-oxo-1-phenyl-2-(pyridin-4-ylamino)ethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)-2-hydroxy-2-phenyl-N-(pyridin-4-yl)acetamide (34 mg, 0.15 mmol), DCC (32 mg, 0.15 mmol), and DMAP (10 mg, 0.08 mmol) were added to 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-oxo-1-phenyl-2-(pyridin-4-ylamino)ethyl 3-amino-6-(1-(1-(tert-butoxycarbonyl)piperidin-4-yl)-1H-pyrazol-4-yl)pyrazine-2-carboxylate (39 mg, 0.065 mmol, mixture).

### Synthesis of (R)-2-oxo-1-phenyl-2-(pyridin-4-ylamino)ethyl 3-amino-6-(1-(piperidin-4-yl)-1H-pyrazol-4-yl)pyrazine-2-carboxylate 2,2,2-trifluoroacetate (TDH-479, step 3)

(R)-2-oxo-1-phenyl-2-(pyridin-4-ylamino)ethyl 3-amino-6-(1-(1-(tert-butoxycarbonyl)piperidin-4-yl)-1H-pyrazol-4-yl)pyrazine-2-carboxylate (30 mg, 0.05 mmol) was dissolved in DCM (1.0 mL), 4 M HCl in dioxane (0.5 mL) was added, and it was reacted at room temperature for 2 hours. The reaction solvent was concentrated to obtain a yellow solid, (R)-2-oxo-1-phenyl-2-(pyridin-4-ylamino)ethyl 3-amino-6-(1-(piperidin-4-yl)-1H-pyrazol-4-yl)pyrazine-2-carboxylate 2,2,2-trifluoroacetate (8 mg, 0.013 mmol, 43%).

### Synthesis of (R)-2-((3,4-difluorophenyl)amino)-2-oxo-1-phenylethyl 3-amino-6-(1-(piperidin-4-yl)-1H-pyrazol-4-yl)pyrazine-2-carboxylate hydrochloride (TDH-488)

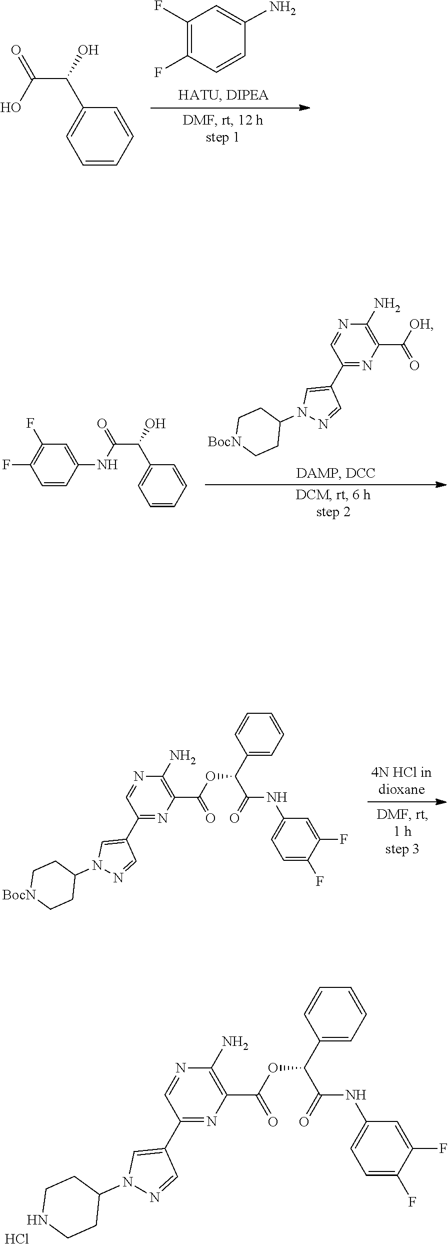

### Synthesis of (R)—N-(3,4-difluorophenyl)-2-hydroxy-2-phenylacetamide (step 1)

(R)-2-hydroxy-2-phenylacetic acid (304 mg, 2.0 mmol) was dissolved in DMF (8 mL). Then, DIPEA (1.7 mL, 6.6 mmol) and HATU (1.14 g, 3.0 mmol) were added, and stirred for 20 minutes. 3,4-difluoroaniline (0.26 mL, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired yellow solid, (R)—N-(3,4-difluorophenyl)-2-hydroxy-2-phenylacetamide (68 mg, 0.26 mmol, 13%).

¹H NMR (400 MHz, CDCl₃) δ 8.41 (s, 1H), 7.68-7.58 (m, 1H), 7.50-7.35 (m, 5H), 7.18-7.02 (m, 2H), 5.16 (d, J=2.8 Hz, 1H), 3.53 (d, J=3.2 Hz, 1H).

### Synthesis of (R)-2-((3,4-difluorophenyl)amino)-2-oxo-1-phenylethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (80 mg, 0.21 mmol) was dissolved in DCM. Then, (R)—N-(3,4-difluorophenyl)-2-hydroxy-2-phenylacetamide (55 mg, 0.21 mmol), DCC (42 mg, 0.21 mmol), and DMAP (13 mg, 0.10 mmol) were added at 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-2-((3,4-difluorophenyl)amino)-2-oxo-1-phenylethyl 3-amino-6-(1-(1-(tert-butoxycarbonyl)piperidin-4-yl)-1H-pyrazol-4-yl)pyrazine-2-carboxylate (92 mg, 0.15 mmol, 69%).

¹H NMR (400 MHz, CDCl₃) δ 9.03 (s, 1H), 8.54 (s, 1H), 8.01 (s, 1H), 7.91 (s, 1H), 7.68-7.61 (m, 3H), 7.47-7.39 (m, 3H), 7.25-7.17 (m, 1H), 7.14-7.05 (m, 1H), 6.45 (s, 1H), 6.36 (s, 2H), 4.40-4.35 (m, 1H), 2.99-2.90 (m, 2H), 2.24-2.17 (m, 2H), 2.02-1.95 (m, 2H), 1.88-1.79 (m, 2H), 1.50 (s, 9H).

### Synthesis of (R)-2-((3,4-difluorophenyl)amino)-2-oxo-1-phenylethyl 3-amino-6-(1-(piperidin-4-yl)-1H-pyrazol-4-yl)pyrazine-2-carboxylate hydrochloride (TDH-488, step 3)

(R)-2-((3,4-difluorophenyl)amino)-2-oxo-1-phenylethyl 3-amino-6-(1-(1-(tert-butoxycarbonyl)piperidin-4-yl)-1H-pyrazol-4-yl)pyrazine-2-carboxylate (30 mg, 0.047 mmol) was dissolved in DCM (1.0 mL), 4 M HCl in dioxane (0.5 mL) was added, and it was reacted at room temperature for 2 hours. The reaction solvent was concentrated to yield a yellow solid, (R)-2-((3,4-difluorophenyl)amino)-2-oxo-1-phenylethyl 3-amino-6-(1-(piperidin-4-yl)-1H-pyrazol-4-yl)pyrazine-2-carboxylate hydrochloride (20 mg, 0.035 mmol, 75%).

### Synthesis of 1-(4-fluorophenyl)-2-((4-fluorophenyl)amino)-2-oxoethyl 3-amino-6-(1-(piperidin-4-yl)-1H-pyrazol-4-yl)pyrazine-2-carboxylate hydrochloride (TDH-489)

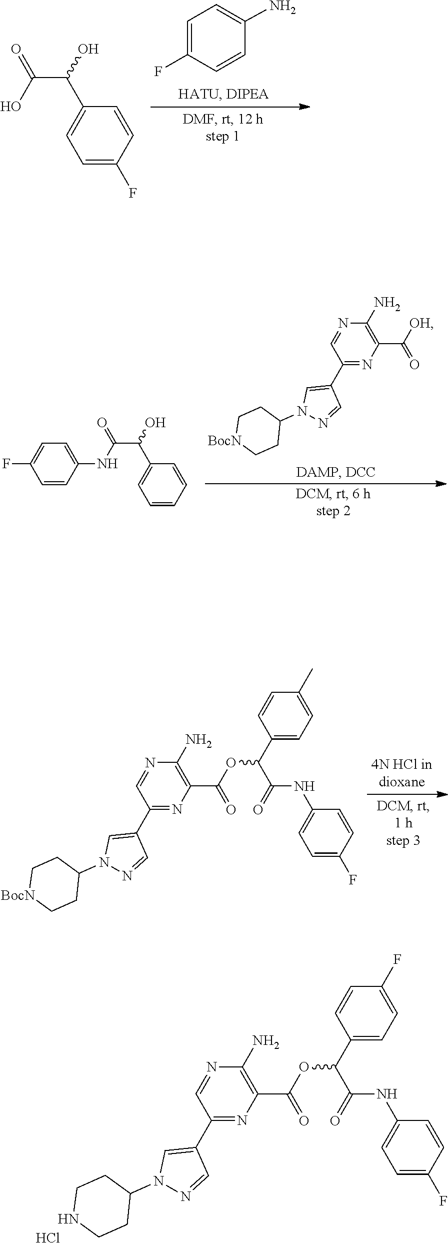

### Synthesis of N,2-bis(4-fluorophenyl)-2-hydroxyacetamide (step 1)

2-(4-fluorophenyl)-2-hydroxyacetic acid (373 mg, 2.0 mmol) was dissolved in DMF (8 mL). Then, DIPEA (1.7 mL, 6.6 mmol) and HATU (1.14 g, 3.0 mmol) were added, and stirred for 20 minutes. 4-fluoroaniline (0.25 mL, 2.6 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired yellow oil N,2-bis(4-fluorophenyl)-2-hydroxyacetamide (50 mg, 0.19 mmol, mixture).

### Synthesis of 2-((4-fluorophenyl)amino)-2-oxo-1-(p-tolyl)ethyl 3-amino-6-(1-(1-(tert-butoxycarbonyl)piperidin-4-yl)-1H-pyrazol-4-yl)pyrazine-2-carboxylate (step 2)

3-amino-6-(1-(1-(tert-butoxycarbonyl)piperidin-4-yl)-1H-pyrazol-4-yl)pyrazine-2-carboxylic acid (80 mg, 0.21 mmol) was dissolved in DCM. Then, N,2-bis(4-fluorophenyl)-2-hydroxyacetamide (55 mg, 0.21 mmol), DCC (42 mg, 0.21 mmol), and DMAP (13 mg, 0.10 mmol) were added at 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, 2-((4-fluorophenyl)amino)-2-oxo-1-(p-tolyl)ethyl 3-amino-6-(1-(1-(tert-butoxycarbonyl)piperidin-4-yl)-1H-pyrazol-4-yl)pyrazine-2-carboxylate (93 mg, 0.15 mmol, 69%).

¹H NMR (400 MHz, CDCl₃) δ 8.99 (s, 1H), 8.54 (s, 1H), 8.01 (s, 1H), 7.89 (s, 1H), 7.66-7.59 (m, 2H), 7.59-7.50 (m, 2H), 7.15-7.09 (m, 2H), 7.08-6.99 (m, 2H), 6.43 (s, 1H), 6.36 (s, 2H), 4.36-4.28 (m, 3H), 2.96-2.90 (m, 2H), 2.21-2.16 (m, 2H), 2.00-1.95 (m, 2H), 1.50 (s, 9H).

### Synthesis of 1-(4-fluorophenyl)-2-((4-fluorophenyl)amino)-2-oxoethyl 3-amino-6-(1-(piperidin-4-yl)-1H-pyrazol-4-yl)pyrazine-2-carboxylate hydrochloride (TDH-489, step 3)

2-((4-fluorophenyl)amino)-2-oxo-1-(p-tolyl)ethyl 3-amino-6-(1-(1-(tert-butoxycarbonyl)piperidin-4-yl)-1H-pyrazol-4-yl)pyrazine-2-carboxylate (30 mg, 0.05 mmol) was dissolved in DCM (1.0 mL), 4 M HCl in dioxane (0.5 mL) was added, and it was reacted at room temperature for 2 hours. The reaction solvent was concentrated to yield a yellow solid, 1-(4-fluorophenyl)-2-((4-fluorophenyl)amino)-2-oxoethyl 3-amino-6-(1-(piperidin-4-yl)-1H-pyrazol-4-yl)pyrazine-2-carboxylate hydrochloride (18 mg, 0.032 mmol, 67%).

### Synthesis of (R)-3-amino-N-(2-((4-fluorophenyl)amino)-2-oxo-1-phenylethyl)-N-methyl-6-(1-(piperidin-4-yl)-1H-pyrazol-4-yl)pyrazine-2-carboxamide hydrochloride (TDH-490)

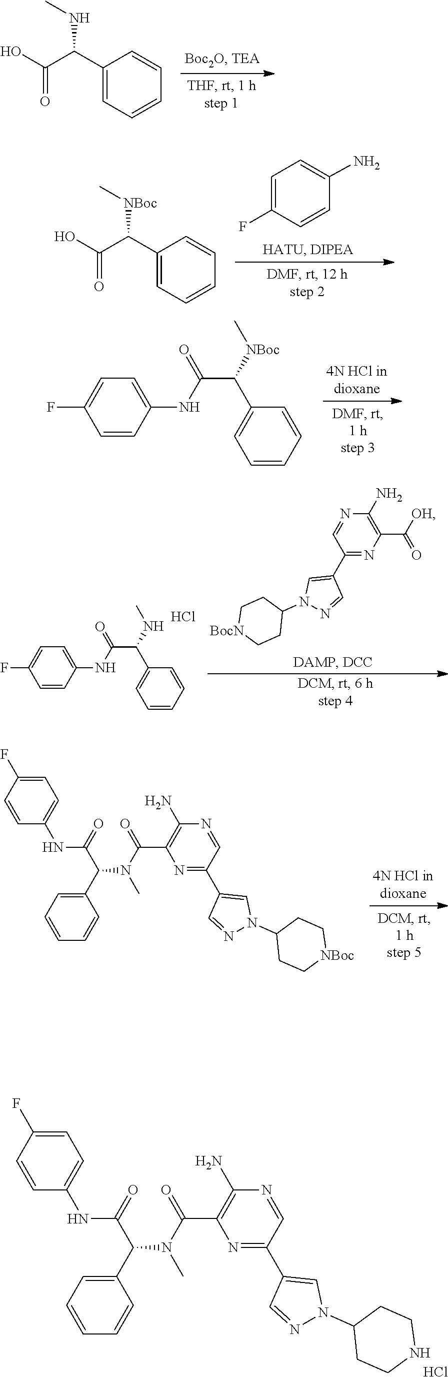

### Synthesis of (R)-2-((tert-butoxycarbonyl)(methyl)amino)-2-phenylacetic acid (step 1)

(R)-2-(methylamino)-2-phenylacetic acid (330 mg, 2 mmol) was dissolved in THF (8 mL), and TEA (0.42 mL, 3.0 mmol) was added. After slowly adding di-tert-butyl dicarbonate (0.55 mL, 2.4 mmol) to the above solution at 0° C., and reacted at room temperature for 1 hour. After concentrating the solvent, the reaction solvent was concentrated, diluted with EtOAc, washed with saturated NaCl aqueous solution, dried over anhydrous MgSO₄, and filtered. The collected organic solvents were concentrated and purified by column chromatography to obtain the desired yellow oil (R)-2-((tert-butoxycarbonyl)(methyl)amino)-2-phenylacetic acid (404 mg, 1.5 mmol, mixture).

### Synthesis of tert-butyl (R)-(2-((4-fluorophenyl)amino)-2-oxo-1-phenylethyl)(methyl)carbamate (step 2)

(R)-2-((tert-butoxycarbonyl)(methyl)amino)-2-phenylacetic acid (404 mg, 1.5 mmol) was dissolved in DMF (6 mL). Then, DIPEA (0.53 mL, 3.0 mmol) and HATU (868 mg, 2.3 mmol) were added and stirred for 20 minutes. 4-fluoroaniline (0.19 mL, 2.0 mmol) was added and reacted overnight at room temperature. The reaction solvent was concentrated, diluted with EtOAc, washed with a saturated aqueous NaCl solution, dried over anhydrous MgSO₄, and filtered. The collected organic solvents were concentrated and purified by column chromatography to obtain the desired white solid, tert-butyl (R)-(2-((4-fluorophenyl)amino)-2-oxo-1-phenylethyl)(methyl)carbamate (69 mg, 0.19 mmol, 13%).

¹H NMR (400 MHz, CDCl₃) δ 7.53-7.47 (m, 2H), 7.44-7.35 (m, 5H), 7.06-6.98 (m, 2H), 5.91 (s, 1H), 2.83 (s, 3H), 1.50 (s, 9H).

### Synthesis of (R)—N-(4-fluorophenyl)-2-(methylamino)-2-phenylacetamide hydrochloride (step 3)

Tert-butyl (R)-(2-((4-fluorophenyl)amino)-2-oxo-1-phenylethyl)(methyl)carbamate (69 mg, 0.19 mmol) was dissolved in DCM (1.0 mL). Then, 4 M HCl in dioxane (0.5 mL) was added, and the mixture was reacted at room temperature for 2 hours. The reaction solvent was concentrated to obtain (R)—N-(4-fluorophenyl)-2-(methylamino)-2-phenylacetamide hydrochloride (60 mg, 0.20 mmol, quant.) as an orange solid.

¹H NMR (500 MHz, DMSO) δ 11.25-11.11 (m, 1H), 9.84 (s, 1H), 9.54 (s, 1H), 7.70-7.61 (m, 4H), 7.56-7.46 (m, 3H), 7.23-7.14 (m, 2H), 5.14 (s, 1H), 3.58 (s, 3H).

### Synthesis of tert-butyl (R)-4-(4-(5-amino-6-((2-((4-fluorophenyl)amino)-2-oxo-1-phenylethyl)(methyl)carbamoyl)pyrazin-2-yl)-1H-pyrazol-1-yl)piperidine-1-carboxylate (step 4)

3-amino-6-(1-(1-(tert-butoxycarbonyl)piperidin-4-yl)-1H-pyrazol-4-yl)pyrazine-2-carboxylic acid (60 mg, 0.15 mmol) was dissolved in DCM. Then, (R)—N-(4-fluorophenyl)-2-(methylamino)-2-phenylacetamide hydrochloride (46 mg, 0.15 mmol), DCC (32 mg, 0.15 mmol), and DMAP (10 mg, 0.08 mmol) were added to 0° C. and reacted overnight at room temperature. After completion of the reaction, the mixture was diluted with DCM, washed with water, dried over anhydrous MgSO₄, filtered and concentrated. The concentrate was separated and purified by silica gel column chromatography to obtain a yellow solid, (R)-1-((4-fluorophenyl)amino)-3-methyl-1-oxobutan-2-yl 3-amino-6-(1-(1-(tert-butoxycarbonyl)piperidin-4-yl)-1H-pyrazol-4-yl)pyrazine-2-carboxylate (60 mg, 0.096 mmol, mixture).

### Synthesis of (R)-3-amino-N-(2-((4-fluorophenyl)amino)-2-oxo-1-phenylethyl)-N-methyl-6-(1-(piperidin-4-yl)-1H-pyrazol-4-yl)pyrazine-2-carboxamide hydrochloride (TDH-490, step 5)

Tert-butyl (R)-4-(4-(5-amino-6-((2-((4-fluorophenyl)amino)-2-oxo-1-phenylethyl)(methyl)carbamoyl)pyrazin-2-yl)-1H-pyrazol-1-yl)piperidine-1-carboxylate (20 mg, 0.03 mmol) was dissolved in DCM (1.0 mL), 4 M HCl in dioxane (0.5 mL) was added, and it was reacted at room temperature for 2 hours. The reaction solvent was concentrated to obtain a yellow solid, (R)-3-amino-N-(2-((4-fluorophenyl)amino)-2-oxo-1-phenylethyl)-N-methyl-6-(1-(piperidin-4-yl)-1H-pyrazol-4-yl)pyrazine-2-carboxamide hydrochloride (8 mg, 0.014 mmol, 47%).

### Synthesis of (R)-2-((4-fluorophenyl)amino)-2-oxo-1-phenylethyl 3-amino-6-(1-(1-((1-(3-(2,4-dioxotetrahydropyrimidin-1(2H)-yl)-4-methoxybenzoyl)piperidin-4-yl)methyl)piperidin-4-yl)-1H-pyrazol-4-yl)pyrazine-2-carboxylate [TDH-445]

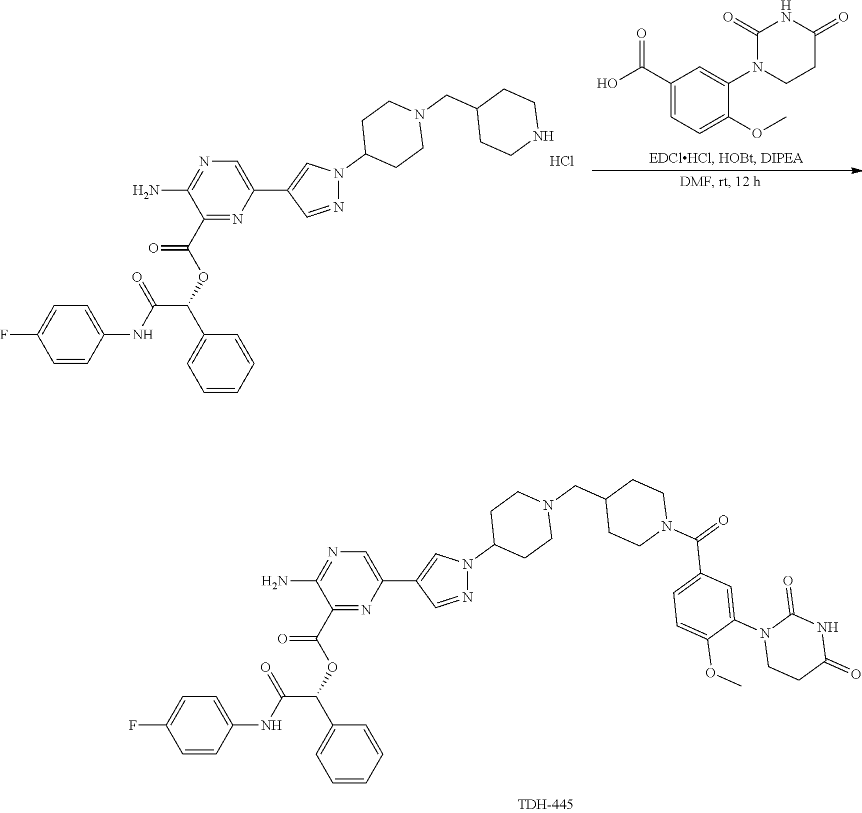 [SMILES: Nc1ncc(-c2cnn(C3CCN(CC4CCNCC4)CC3)c2)nc1C(=O)O[C@@H](C(=O)Nc1ccc(F)cc1)c1ccccc1 ; Cl ; COc1ccc(C(=O)O)cc1N1CCC(=O)NC1=O ; COc1ccc(C(=O)N2CCC(CN3CCC(n4cc(-c5cnc(N)c(C(=O)O[C@@H](C(=O)Nc6ccc(F)cc6)c6ccccc6)n5)cn4)CC3)CC2)cc1N1CCC(=O)NC1=O]

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (15 mg, 0.023 mmol, 1 eq.), 3-(2,4-dioxotetrahydropyrimidin-1(2H)-yl)-4-methoxybenzoic acid (7 mg, 0.023 mmol, 1.0 eq.), EDCI-HCl (5 mg, 0.025 mmol, 1.1 eq.), HOBt (4 mg, 0.025 mmol, 1.1 eq.), and DIPEA (0.02 mL, 0.12 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(3-(2,4-dioxotetrahydropyrimidin-1(2H)-yl)-4-methoxybenzoyl)piperidin-4-yl)methyl)piperidin-4-yl)-1H-pyrazol-4-yl)pyrazine-2-carboxylate (10 mg, 51%).

### Synthesis of (R)-2-((4-fluorophenyl)amino)-2-oxo-1-phenylethyl 3-amino-6-(1-(1-((1-(2-(1-(2-(1-methyl-2,6-dioxopiperidin-3-yl)-1,3-dioxoisoindolin-5-yl)piperidin-4-yl)acetyl)piperidin-4-yl)methyl)piperidin-4-yl)-1H-pyrazol-4-yl)pyrazine-2-carboxylate [TDH-450]

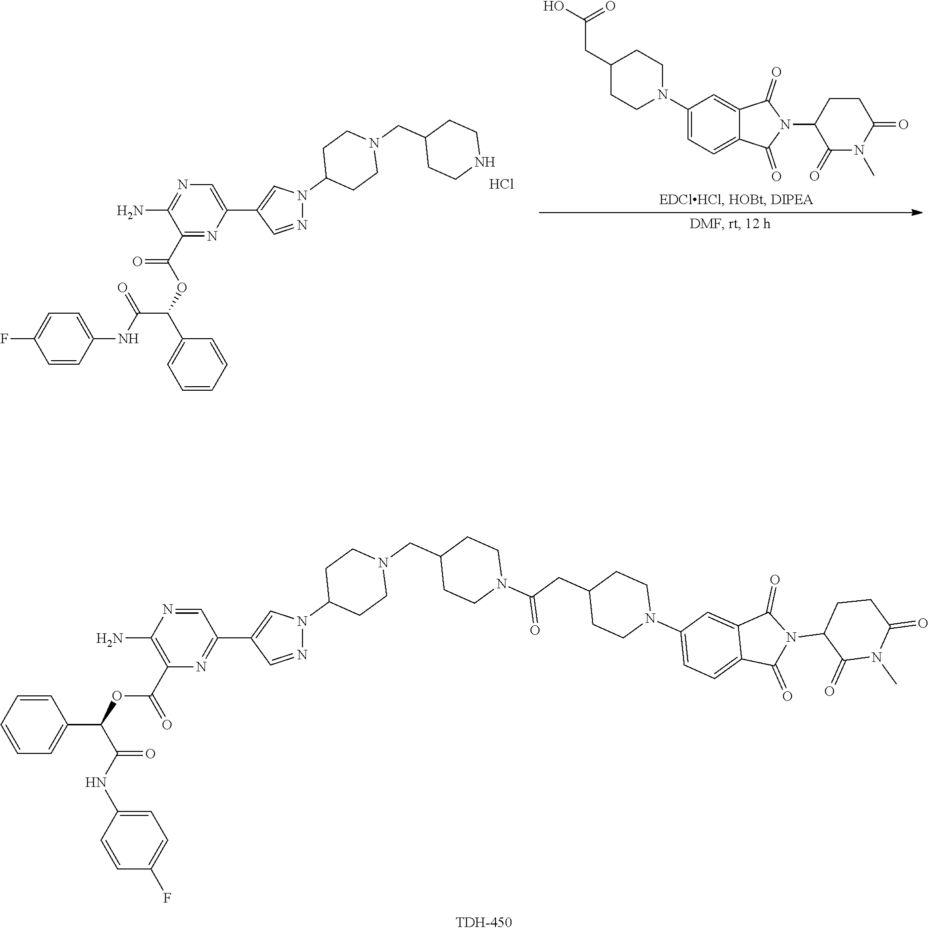 [SMILES: Nc1ncc(-c2cnn(C3CCN(CC4CCNCC4)CC3)c2)nc1C(=O)O[C@@H](C(=O)Nc1ccc(F)cc1)c1ccccc1 ; Cl ; CN1C(=O)CCC(N2C(=O)c3ccc(N4CCC(CC(=O)O)CC4)cc3C2=O)C1=O ; CN1C(=O)CCC(N2C(=O)c3ccc(N4CCC(CC(=O)N5CCC(CN6CCC(n7cc(-c8cnc(N)c(C(=O)O[C@@H](C(=O)Nc9ccc(F)cc9)c9ccccc9)n8)cn7)CC6)CC5)CC4)cc3C2=O)C1=O]

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (50 mg, 0.08 mmol, 1 eq.), 2-(1-(2-(1-methyl-2,6-dioxopiperidin-3-yl)-1,3-dioxoisoindolin-5-yl)piperidin-4-yl)acetic acid (32 mg, 0.08 mmol, 1.0 eq.), EDCI-HCl (16 mg, 0.09 mmol, 1.1 eq.), HOBt (13 mg, 0.09 mmol, 1.1 eq.), and DIPEA (0.07 mL, 0.39 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(2-(1-(2-(1-methyl-2,6-dioxopiperidin-3-yl)-1,3-dioxoisoindolin-5-yl)piperidin-4-yl)acetyl)piperidin-4-yl)methyl)piperidin-4-yl)-1H-pyrazol-4-yl)pyrazine-2-carboxylate (13 mg, 16%).

### Synthesis of (R)-2-((4-fluorophenyl)amino)-2-oxo-1-phenylethyl 3-amino-6-(1-(1-((1-(1-(3-(2,4-dioxotetrahydropyrimidin-1 (2H)-yl)-4-methoxybenzoyl)piperidine-4-carbonyl)piperidin-4-yl)methyl)piperidin-4-yl)-1H-pyrazol-4-yl)pyrazine-2-carboxylate [TDH-515]

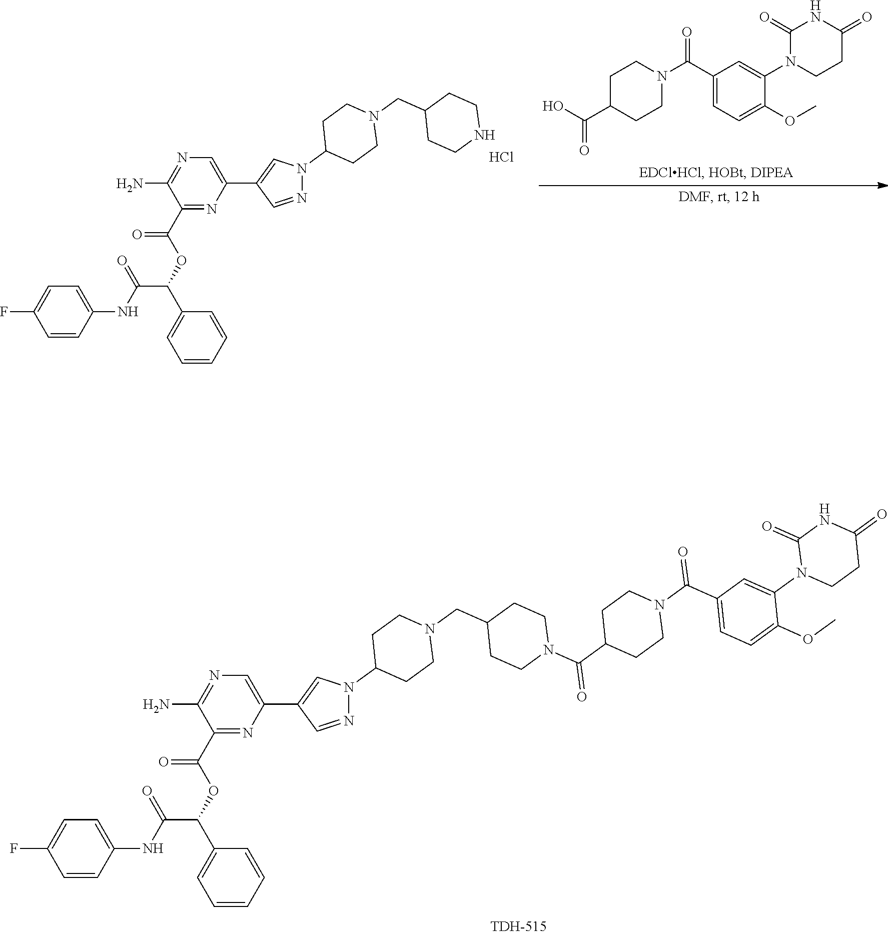 [SMILES: Nc1ncc(-c2cnn(C3CCN(CC4CCNCC4)CC3)c2)nc1C(=O)O[C@@H](C(=O)Nc1ccc(F)cc1)c1ccccc1 ; Cl ; COc1ccc(C(=O)N2CCC(C(=O)O)CC2)cc1N1CCC(=O)NC1=O ; COc1ccc(C(=O)N2CCC(C(=O)N3CCC(CN4CCC(n5cc(-c6cnc(N)c(C(=O)O[C@@H](C(=O)Nc7ccc(F)cc7)c7ccccc7)n6)cn5)CC4)CC3)CC2)cc1N1CCC(=O)NC1=O]

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (20 mg, 0.031 mmol, 1 eq.), 1-(3-(2,4-dioxotetrahydropyrimidin-1(2H)-yl)-4-methoxybenzoyl)piperidine-4-carboxylic acid (12 mg, 0.031 mmol, 1.0 eq.), EDCI-HCl (7 mg, 0.034 mmol, 1.1 eq.), HOBt (5 mg, 0.034 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(1-(3-(2,4-dioxotetrahydropyrimidin-1(2H)-yl)-4-methoxybenzoyl)piperidine-4-carbonyl)piperidin-4-yl)methyl)piperidin-4-yl)-1H-pyrazol-4-yl)pyrazine-2-carboxylate (7 mg, 23%).

### Synthesis of (R)-2-((4-fluorophenyl)amino)-2-oxo-1-phenylethyl 3-amino-6-(1-(1-((1-(2-(3-((1-(2,6-dioxopiperidin-3-yl)-2,5-dioxo-2,5-dihydro-1H-pyrrol-3-yl)amino)phenyl)acetyl)piperidin-4-yl)methyl)piperidin-4-yl)-1H-pyrazol-4-yl)pyrazine-2-carboxylate [TDH-516]

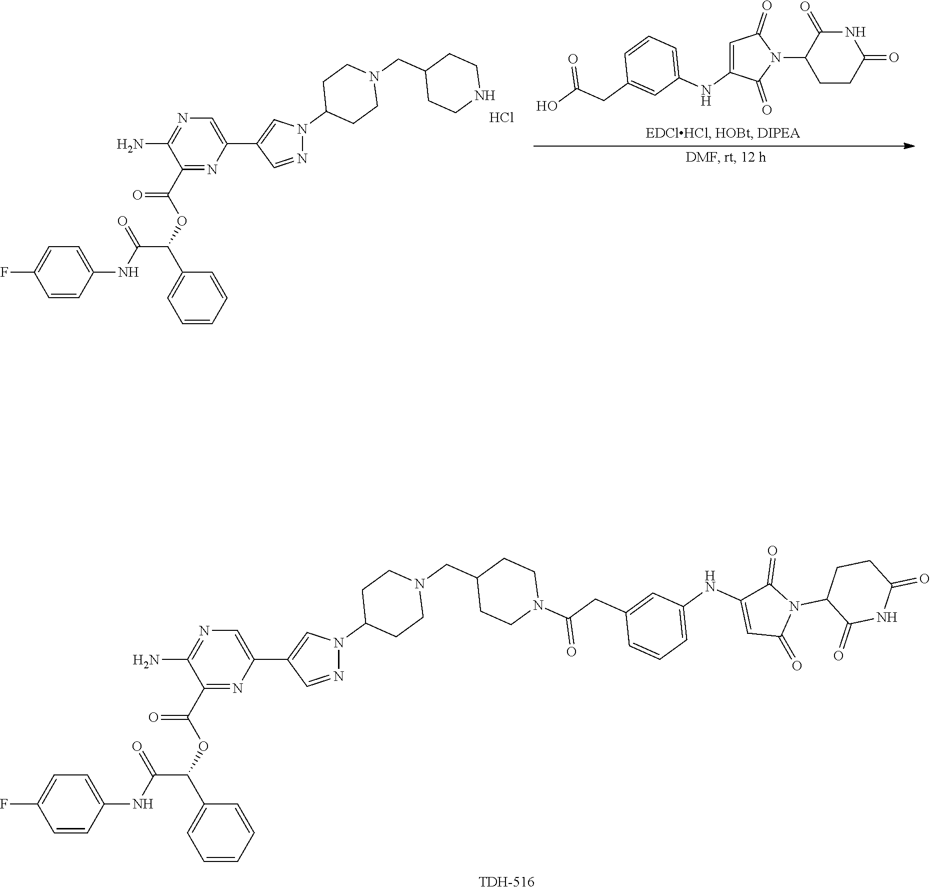 [SMILES: Nc1ncc(-c2cnn(C3CCN(CC4CCNCC4)CC3)c2)nc1C(=O)O[C@@H](C(=O)Nc1ccc(F)cc1)c1ccccc1 ; Cl ; O=C(O)Cc1cccc(NC2=CC(=O)N(C3CCC(=O)NC3=O)C2=O)c1 ; Nc1ncc(-c2cnn(C3CCN(CC4CCN(C(=O)Cc5cccc(NC6=CC(=O)N(C7CCC(=O)NC7=O)C6=O)c5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (20 mg, 0.031 mmol, 1 eq.), 2-(3-((1-(2,6-dioxopiperidin-3-yl)-2,5-dioxo-2,5-dihydro-1H-pyrrol-3-yl)amino)phenyl)acetic acid (11 mg, 0.031 mmol, 1.0 eq.), EDCI-HCl (7 mg, 0.034 mmol, 1.1 eq.), HOBt (5 mg, 0.034 mmol, 1.1 eq.), and DIPEA (0.03 mL, 0.15 mmol, 5 eq.) were dissolved in DMF (1.0 mL), and the mixture was reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(2-(3-((1-(2,6-dioxopiperidin-3-yl)-2,5-dioxo-2,5-dihydro-1H-pyrrol-3-yl)amino)phenyl)acetyl)piperidin-4-yl)methyl)piperidin-4-yl)-1H-pyrazol-4-yl)pyrazine-2-carboxylate (14 mg, 50%).

### Synthesis of (R)-2-((4-fluorophenyl)amino)-2-oxo-1-phenylethyl 3-amino-6-(1-(1-((1-(2-(2,6-dioxopiperidin-3-yl)-6-fluoro-1,3-dioxoisoindolin-5-yl)piperidin-4-yl)methyl)piperidin-4-yl)-1H-pyrazol-4-yl)pyrazine-2-carboxylate [TDH-519]

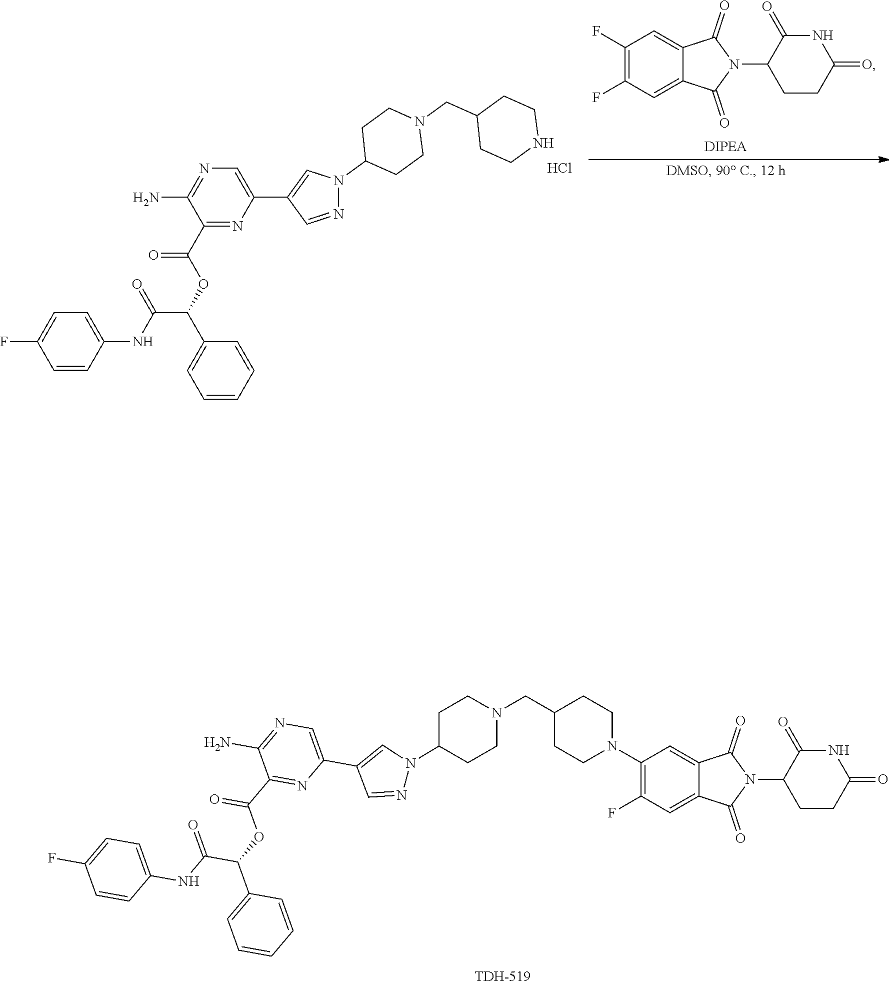 [SMILES: Nc1ncc(-c2cnn(C3CCN(CC4CCNCC4)CC3)c2)nc1C(=O)O[C@@H](C(=O)Nc1ccc(F)cc1)c1ccccc1 ; Cl ; O=C1CCC(N2C(=O)c3cc(F)c(F)cc3C2=O)C(=O)N1 ; Nc1ncc(-c2cnn(C3CCN(CC4CCN(c5cc6c(cc5F)C(=O)N(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (20 mg, 0.031 mmol, 1 eq.), 2-(2,6-dioxopiperidin-3-yl)-5,6-difluoroisoindoline-1,3-dione (9 mg, 0.029 mmol, 0.95 eq.) and DIPEA (0.02 mL, 0.092 mmol, 3 eq.) were dissolved in DMSO (1.0 mL), and the mixture was reacted at 90° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(2-(2,6-dioxopiperidin-3-yl)-6-fluoro-1,3-dioxoisoindolin-5-yl)piperidin-4-yl)methyl)piperidin-4-yl)-1H-pyrazol-4-yl)pyrazine-2-carboxylate (10 mg, 38%).

### Synthesis of (R)-2-((4-fluorophenyl)amino)-2-oxo-1-phenylethyl 3-amino-6-(1-(1-((1-(1-(2,6-dioxopiperidin-3-yl)-2,5-dioxo-2,5-dihydro-1H-pyrrol-3-yl)piperidin-4-yl)methyl)piperidin-4-yl)-1H-pyrazol-4-yl)pyrazine-2-carboxylate [TDH-521]

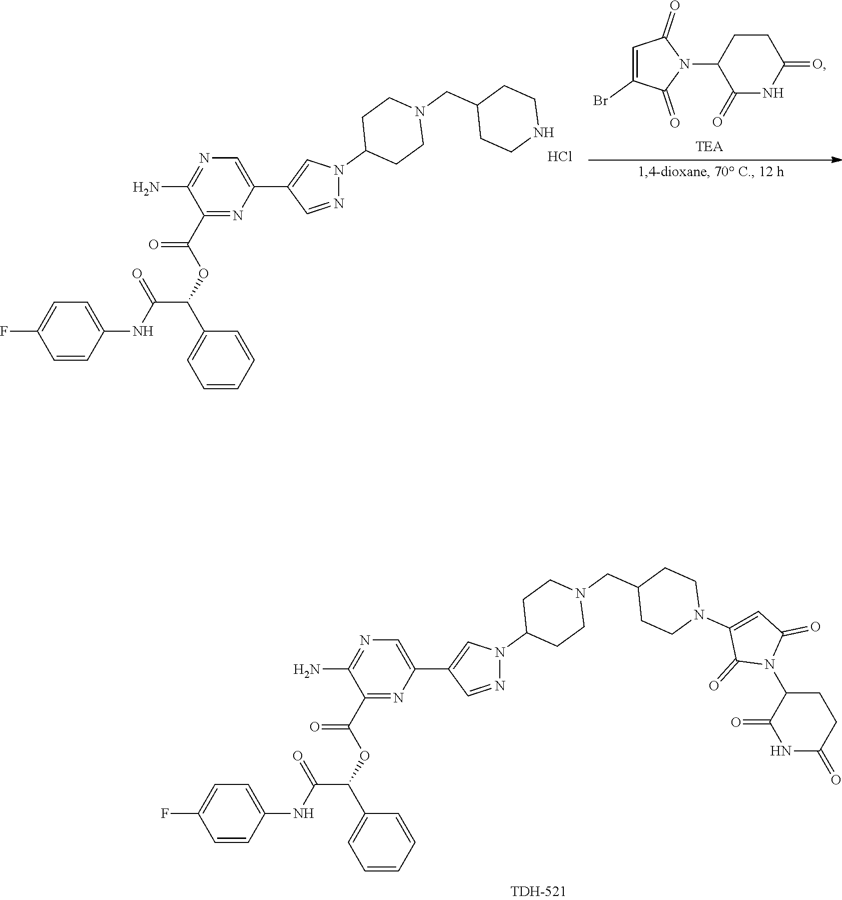 [SMILES: Nc1ncc(-c2cnn(C3CCN(CC4CCNCC4)CC3)c2)nc1C(=O)O[C@@H](C(=O)Nc1ccc(F)cc1)c1ccccc1 ; Cl ; O=C1CCC(N2C(=O)C=C(Br)C2=O)C(=O)N1 ; Nc1ncc(-c2cnn(C3CCN(CC4CCN(C5=CC(=O)N(C6CCC(=O)NC6=O)C5=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (68 mg, 0.11 mol, 1.5 eq.), 3-(3-bromo-2,5-dioxo-2,5-dihydro-1H-pyrrol-1-yl)piperidine-2,6-dione (20 mg, 0.07 mmol, 1.0 eq.) and TEA (0.03 mL, 0.21 mmol, 3 eq.) were dissolved in 1,4-dioxane (1.0 mL), and reacted overnight at 70° C. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(1-(2,6-dioxopiperidin-3-yl)-2,5-dioxo-2,5-dihydro-1H-pyrrol-3-yl)piperidin-4-yl)methyl)piperidin-4-yl)-1H-pyrazol-4-yl)pyrazine-2-carboxylate (13 mg, 23%).

### Synthesis of (R)-2-((4-fluorophenyl)amino)-2-oxo-1-phenylethyl 3-amino-6-(1-(1-((1-(1-(1-(2,6-dioxopiperidin-3-yl)-2,5-dioxo-2,5-dihydro-1H-pyrrol-3-yl)piperidine-4-carbonyl)piperidin-4-yl)methyl)piperidin-4-yl)-1H-pyrazol-4-yl)pyrazine-2-carboxylate [TDH-523]

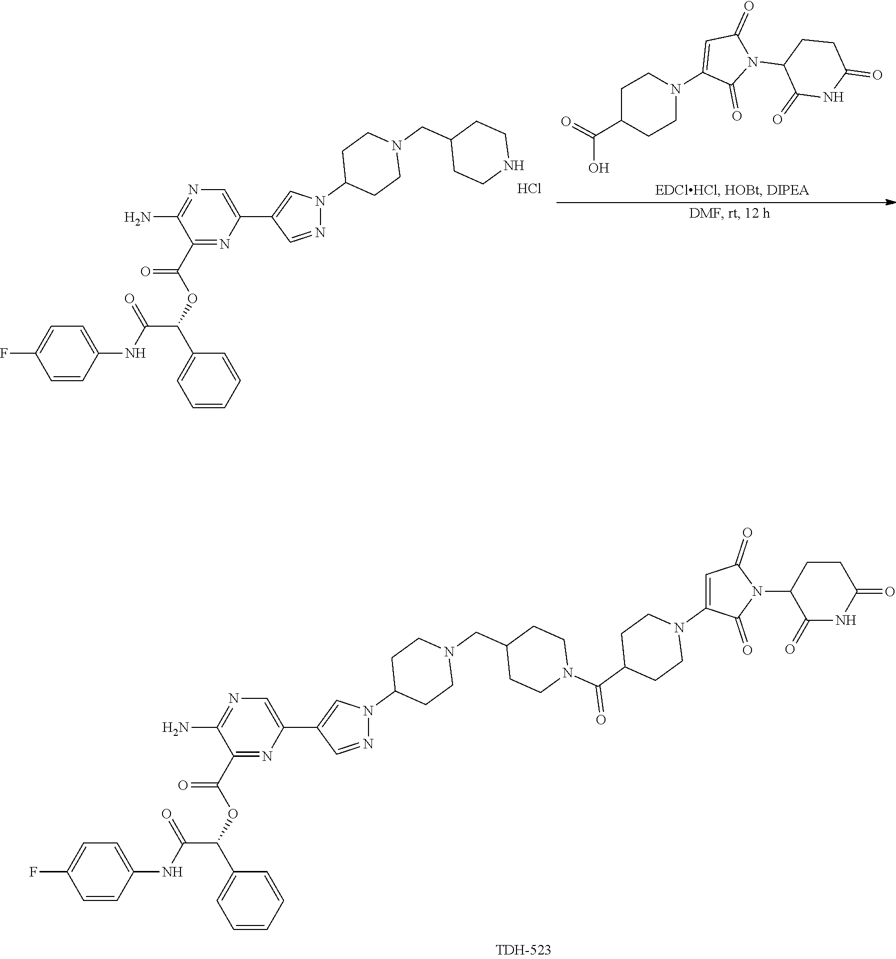 [SMILES: Nc1ncc(-c2cnn(C3CCN(CC4CCNCC4)CC3)c2)nc1C(=O)O[C@@H](C(=O)Nc1ccc(F)cc1)c1ccccc1 ; Cl ; O=C1CCC(N2C(=O)C=C(N3CCC(C(=O)O)CC3)C2=O)C(=O)N1 ; Nc1ncc(-c2cnn(C3CCN(CC4CCN(C(=O)C5CCN(C6=CC(=O)N(C7CCC(=O)NC7=O)C6=O)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (20 mg, 0.031 mmol, 1 eq.), 1-(1-(2,6-dioxopiperidin-3-yl)-2,5-dioxo-2,5-dihydro-1H-pyrrol-3-yl)piperidine-4-carboxylic acid (10 mg, 0.031 mmol, 1.0 eq.), EDCI-HCl (7 mg, 0.034 mmol, 1.1 eq.), HOBt (5 mg, 0.034 mmol, 1.1 eq.) and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(2-(3-((1-(2,6-dioxopiperidin-3-yl)-2,5-dioxo-2,5-dihydro-1H-pyrrol-3-yl)amino)phenyl)acetyl)piperidin-4-yl)methyl)piperidin-4-yl)-1H-pyrazol-4-yl)pyrazine-2-carboxylate (5 mg, 18%).

### Synthesis of (R)-2-((4-fluorophenyl)amino)-2-oxo-1-phenylethyl 3-amino-6-(1-(1-((1-((1-(2-(2,6-dioxopiperidin-3-yl)-1,3-dioxoisoindolin-5-yl)piperidin-4-yl)methyl)piperidin-4-yl)methyl)piperidin-4-yl)-1H-pyrazol-4-yl)pyrazine-2-carboxylate [TDH-473]

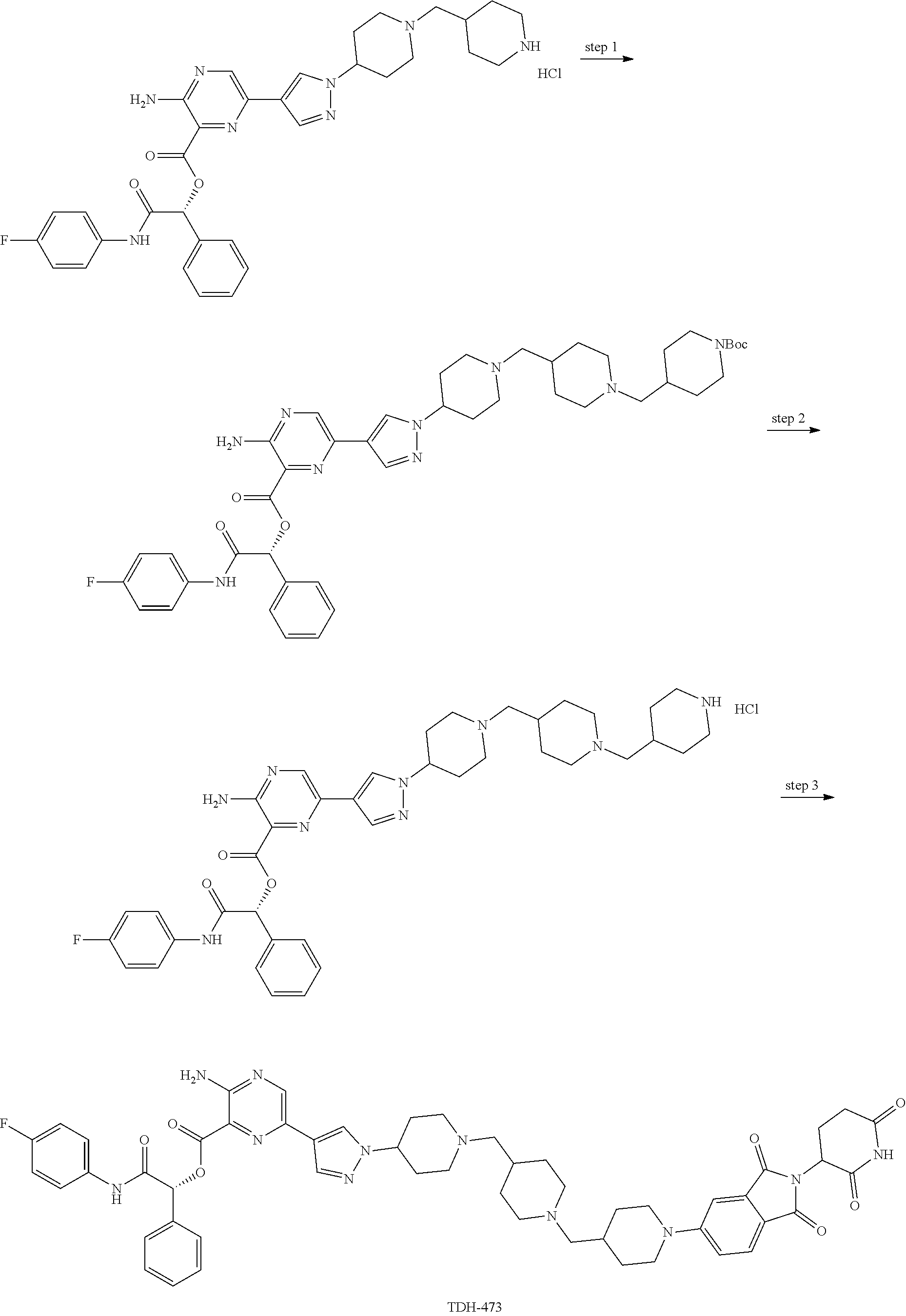 [SMILES: Nc1ncc(-c2cnn(C3CCN(CC4CCNCC4)CC3)c2)nc1C(=O)O[C@@H](C(=O)Nc1ccc(F)cc1)c1ccccc1 ; Cl ; CC(C)(C)OC(=O)N1CCC(CN2CCC(CN3CCC(n4cc(-c5cnc(N)c(C(=O)O[C@@H](C(=O)Nc6ccc(F)cc6)c6ccccc6)n5)cn4)CC3)CC2)CC1 ; Nc1ncc(-c2cnn(C3CCN(CC4CCN(CC5CCNCC5)CC4)CC3)c2)nc1C(=O)O[C@@H](C(=O)Nc1ccc(F)cc1)c1ccccc1 ; Cl ; Nc1ncc(-c2cnn(C3CCN(CC4CCN(CC5CCN(c6ccc7c(c6)C(=O)N(C6CCC(=O)NC6=O)C7=O)CC5)CC4)CC3)c2)nc1C(=O)O[C@@H](C(=O)Nc1ccc(F)cc1)c1ccccc1]

### (R)-2-((4-fluorophenyl)amino)-2-oxo-1-phenylethyl 3-amino-6-(1-(1-((1-((1-(tert-butoxycarbonyl)piperidin-4-yl)methyl)piperidin-4-yl)methyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (100 mg, 0.15 mmol, 1 eq.) was dissolved in DCM (1.0 mL) and DIPEA (0.04 mL, 0.23 mmol, 1.5 eq.) was added at 0° C. After 10 minutes, tert-butyl 4-formylpiperidine-1-carboxylate (50 mg, 0.23 mmol, 1.5 eq.) was added to the above solution and reacted at room temperature for 4 hours. After adding NaBH(OAc)₃ (44 mg, 0.21 mmol, 1.34 eq.) at 0° C., the mixture was reacted overnight at room temperature. After completion of the reaction, the mixture was diluted with water and extracted with DCM. The combined organic layer was filtered after removing the residual water through anhydrous Na₂SO₄. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-((1-(tert-butoxycarbonyl)piperidin-4-yl)methyl)piperidin-4-yl)methyl)piperidin-4-yl)-1H-pyrazol-4-yl)pyrazine-2-carboxylate (99 mg, 81%).

¹H NMR (300 MHz, Chloroform-d) δ 8.99 (s, 1H), 8.53 (s, 1H), 8.02-7.97 (m, 1H), 7.97-7.90 (m, 1H), 7.70-7.62 (m, 2H), 7.62-7.53 (m, 2H), 7.50-7.37 (m, 3H), 7.11-6.99 (m, 2H), 6.44 (s, 1H), 6.36 (s, 2H), 4.27-4.04 (m, 4H), 3.42-3.14 (m, 3H), 3.07-2.96 (m, 3H), 2.79-2.68 (m, 3H), 2.62-2.49 (m, 2H), 2.38-2.29 (m, 3H), 2.24-2.14 (m, 4H), 1.93-1.83 (m, 4H), 1.76-1.65 (m, 2H), 1.47 (s, 9H), 1.31-1.25 (m, 1H), 1.25-1.21 (m, 1H), 1.20-1.16 (m, 1H).

### (R)-2-((4-fluorophenyl)amino)-2-oxo-1-phenylethyl 3-amino-6-(1-(1-((1-(piperidin-4-ylmethyl)piperidin-4-yl)methyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-((1-((1-(tert-butoxycarbonyl)piperidin-4-yl)methyl)piperidin-4-yl)methyl)piperidin-4-yl)-1H-pyrazol-4-yl)pyrazine-2-carboxylate (99 mg, 0.12 mmol) was dissolved in DCM (1.0 mL). Then, 4 N HCl in dioxane (0.5 mL) was slowly added and reacted at room temperature for 1 hour. After completion of the reaction, the precipitate was filtered to obtain (R)-2-((4-fluorophenyl)amino)-2-oxo-1-phenylethyl 3-amino-6-(1-(1-((1-(piperidin-4-ylmethyl)piperidin-4-yl)methyl)piperidin-4-yl)-1H-pyrazol-4-yl)pyrazine-2-carboxylate hydrochloride (78 mg, 87%).

¹H NMR (300 MHz, MeOD) δ 10.40 (s, 1H), 8.62 (s, 1H), 8.28 (s, 1H), 8.09 (s, 1H), 7.78-7.70 (m, 2H), 7.63-7.56 (m, 2H), 7.52-7.42 (m, 3H), 7.14-7.01 (m, 3H), 6.35 (s, 1H), 3.90-3.82 (m, 2H), 3.80-3.72 (m, 2H), 3.58-3.55 (m, 1H), 3.52-3.48 (m, 1H), 3.47-3.41 (m, 2H), 3.27-3.19 (m, 2H), 3.19-3.04 (m, 6H), 2.49-2.44 (m, 2H), 2.40-2.25 (m, 3H), 2.21-2.10 (m, 4H), 1.90-1.81 (m, 2H), 1.60-1.56 (m, 1H), 1.56-1.53 (m, 1H).

### Synthesis of (R)-2-((4-fluorophenyl)amino)-2-oxo-1-phenylethyl 3-amino-6-(1-(1-((1-((1-(2-(2,6-dioxopiperidin-3-yl)-1,3-dioxoisoindolin-5-yl)piperidin-4-yl)methyl)piperidin-4-yl)methyl)piperidin-4-yl)-1H-pyrazol-4-yl)pyrazine-2-carboxylate [step 3, TDH-473]

(R)-2-((4-fluorophenyl)amino)-2-oxo-1-phenylethyl 3-amino-6-(1-(1-((1-(piperidin-4-ylmethyl)piperidin-4-yl)methyl)piperidin-4-yl)-1H-pyrazol-4-yl)pyrazine-2-carboxylate hydrochloride (20 mg, 0.027 mmol, 1 eq.), 2-(2,6-dioxopiperidin-3-yl)-5-fluoroisoindoline-1,3-dione (7 mg, 0.026 mmol, 1 eq.) and DIPEA (0.013 mL, 0.077 mmol, 3 eq.) were dissolved in DMSO (1.0 mL) and reacted at 90° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-((1-(2-(2,6-dioxopiperidin-3-yl)-1,3-dioxoisoindolin-5-yl)piperidin-4-yl)methyl)piperidin-4-yl)methyl)piperidin-4-yl)-1H-pyrazol-4-yl)pyrazine-2-carboxylate (10 mg, 41%).

### Synthesis of (R)-2-((4-fluorophenyl)amino)-2-oxo-1-phenylethyl 3-amino-6-(1-(1-((1-((1-(3-(2,4-dioxotetrahydropyrimidin-1(2H)-yl)-4-methoxybenzoyl)piperidin-4-yl)methyl)piperidin-4-yl)methyl)piperidin-4-yl)-1H-pyrazol-4-yl)pyrazine-2-carboxylate [TDH-477]

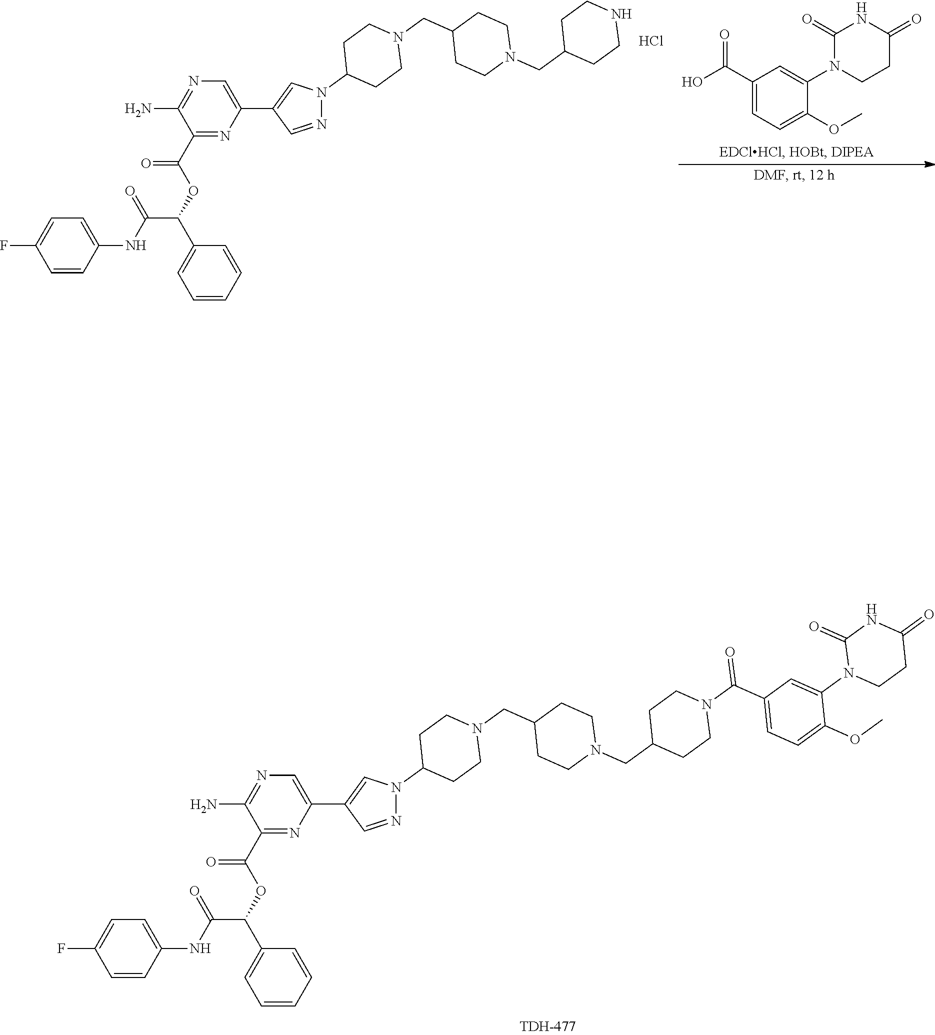 [SMILES: Nc1ncc(-c2cnn(C3CCN(CC4CCN(CC5CCNCC5)CC4)CC3)c2)nc1C(=O)O[C@@H](C(=O)Nc1ccc(F)cc1)c1ccccc1 ; Cl ; COc1ccc(C(=O)O)cc1N1CCC(=O)NC1=O ; COc1ccc(C(=O)N2CCC(CN3CCC(CN4CCC(n5cc(-c6cnc(N)c(C(=O)O[C@@H](C(=O)Nc7ccc(F)cc7)c7ccccc7)n6)cn5)CC4)CC3)CC2)cc1N1CCC(=O)NC1=O]

(R)-2-((4-fluorophenyl)amino)-2-oxo-1-phenylethyl 3-amino-6-(1-(1-((1-(piperidin-4-ylmethyl)piperidin-4-yl)methyl)piperidin-4-yl)-1H-pyrazol-4-yl)pyrazine-2-carboxylate hydrochloride (15 mg, 0.020 mmol, 1 eq.), 3-(2,4-dioxotetrahydropyrimidin-1(2H)-yl)-4-methoxybenzoic acid (5 mg, 0.020 mmol, 1.0 eq.), EDCI-HCl (5 mg, 0.022 mmol, 1.1 eq.), HOBt (4 mg, 0.022 mmol, 1.1 eq.), and DIPEA (0.02 mL, 0.10 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure and separated and purified by silica gel column chromatography to obtain a yellow solid, (R)-2-((4-fluorophenyl)amino)-2-oxo-1-phenylethyl 3-amino-6-(1-(1-((1-((1-(3-(2,4-dioxotetrahydropyrimidin-1(2H)-yl)-4-methoxybenzoyl)piperidin-4-yl)methyl)piperidin-4-yl)methyl)piperidin-4-yl)-1H-pyrazol-4-yl)pyrazine-2-carboxylate (10 mg, 52%).

### Synthesis of (R)-2-((4-fluorophenyl)amino)-2-oxo-1-phenylethyl 3-amino-6-(1-(1-((1-((1-(1-(1-(2,6-dioxopiperidin-3-yl)-2,5-dioxo-2,5-dihydro-1H-pyrrol-3-yl)piperidine-4-carbonyl)piperidin-4-yl)methyl)piperidin-4-yl)methyl)piperidin-4-yl)-1H-pyrazol-4-yl)pyrazine-2-carboxylate [TDH-527]

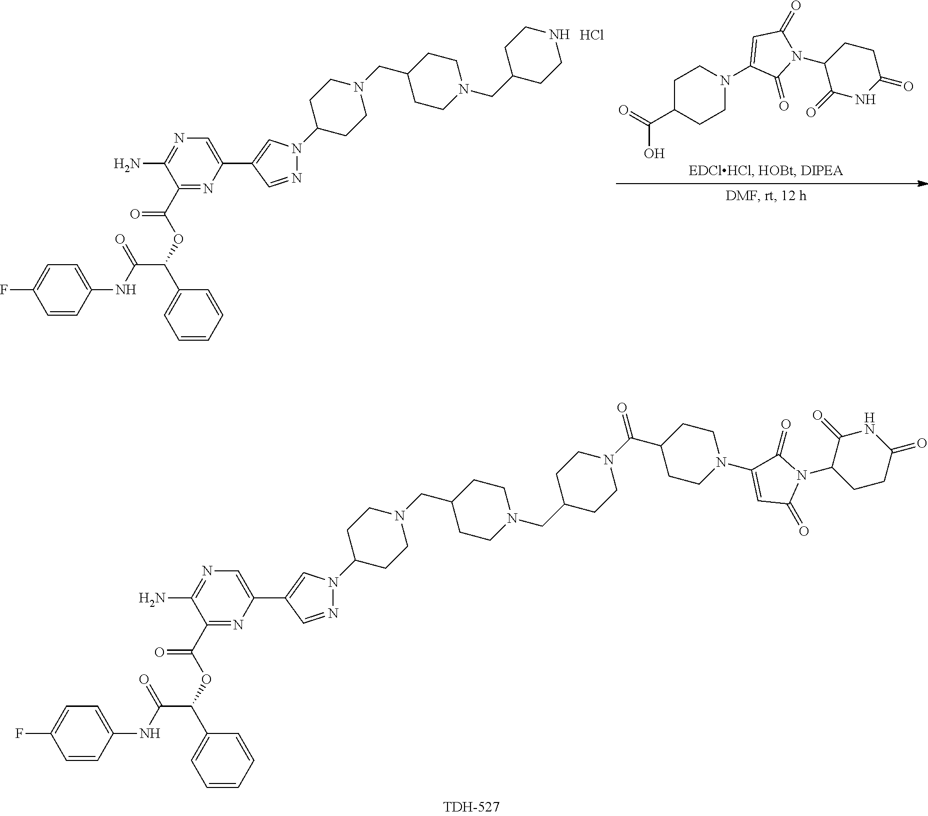 [SMILES: Nc1ncc(-c2cnn(C3CCN(CC4CCN(CC5CCNCC5)CC4)CC3)c2)nc1C(=O)O[C@@H](C(=O)Nc1ccc(F)cc1)c1ccccc1 ; Cl ; O=C1CCC(N2C(=O)C=C(N3CCC(C(=O)O)CC3)C2=O)C(=O)N1 ; Nc1ncc(-c2cnn(C3CCN(CC4CCN(CC5CCN(C(=O)C6CCN(C7=CC(=O)N(C8CCC(=O)NC8=O)C7=O)CC6)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-((1-(piperidin-4-ylmethyl)piperidin-4-yl)methyl)piperidin-4-yl)-1H-pyrazol-4-yl)pyrazine-2-carboxylate hydrochloride (20 mg, 0.027 mmol, 1 eq.), 1-(1-(2,6-dioxopiperidin-3-yl)-2,5-dioxo-2,5-dihydro-1H-pyrrol-3-yl)piperidine-4-carboxylic acid (9 mg, 0.027 mmol, 1.0 eq.), EDCI-HCl (6 mg, 0.030 mmol, 1.1 eq.), HOBt (5 mg, 0.030 mmol, 1.1 eq.), and DIPEA (0.02 mL, 0.14 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure and separated and purified by silica gel column chromatography to obtain a yellow solid, (R)-2-((4-fluorophenyl)amino)-2-oxo-1-phenylethyl 3-amino-6-(1-(1-((1-((1-(1-(1-(2,6-dioxopiperidin-3-yl)-2,5-dioxo-2,5-dihydro-1H-pyrrol-3-yl)piperidine-4-carbonyl)piperidin-4-yl)methyl)piperidin-4-yl)methyl)piperidin-4-yl)-1H-pyrazol-4-yl)pyrazine-2-carboxylate (6 mg, 19%).

### Synthesis of (R)-2-((4-fluorophenyl)amino)-2-oxo-1-phenylethyl 3-amino-6-(1-(1-((1-((1-(1-(1-(2,6-dioxopiperidin-3-yl)-2,5-dioxo-2,5-dihydro-1H-pyrrol-3-yl)azetidine-3-carbonyl)piperidin-4-yl)methyl)piperidin-4-yl)methyl)piperidin-4-yl)-1H-pyrazol-4-yl)pyrazine-2-carboxylate [TDH-533]

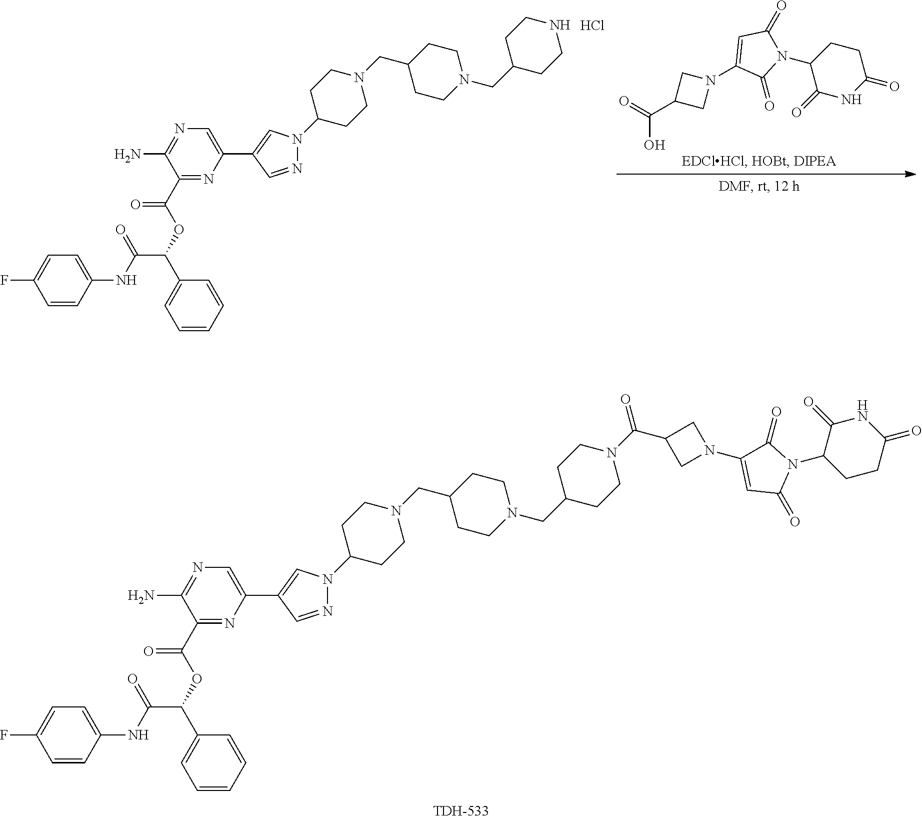 [SMILES: Nc1ncc(-c2cnn(C3CCN(CC4CCN(CC5CCNCC5)CC4)CC3)c2)nc1C(=O)O[C@@H](C(=O)Nc1ccc(F)cc1)c1ccccc1 ; Cl ; O=C1CCC(N2C(=O)C=C(N3CC(C(=O)O)C3)C2=O)C(=O)N1 ; Nc1ncc(-c2cnn(C3CCN(CC4CCN(CC5CCN(C(=O)C6CN(C7=CC(=O)N(C8CCC(=O)NC8=O)C7=O)C6)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-((1-(piperidin-4-ylmethyl)piperidin-4-yl)methyl)piperidin-4-yl)-1H-pyrazol-4-yl)pyrazine-2-carboxylate hydrochloride (20 mg, 0.027 mmol, 1 eq.), 1-(1-(2,6-dioxopiperidin-3-yl)-2,5-dioxo-2,5-dihydro-1H-pyrrol-3-yl)azetidine-3-carboxylic acid (8 mg, 0.027 mmol, 1.0 eq.), EDCI-HCl (6 mg, 0.030 mmol, 1.1 eq.), HOBt (5 mg, 0.030 mmol, 1.1 eq.), and DIPEA (0.02 mL, 0.14 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure and separated and purified by silica gel column chromatography to obtain a yellow solid, (R)-2-((4-fluorophenyl)amino)-2-oxo-1-phenylethyl 3-amino-6-(1-(1-((1-((1-(1-(1-(2,6-dioxopiperidin-3-yl)-2,5-dioxo-2,5-dihydro-1H-pyrrol-3-yl)azetidine-3-carbonyl)piperidin-4-yl)methyl)piperidin-4-yl)methyl)piperidin-4-yl)-1H-pyrazol-4-yl)pyrazine-2-carboxylate (5 mg, 22%).

### Synthesis of (R)-2-((4-fluorophenyl)amino)-2-oxo-1-phenylethyl 3-amino-6-(1-(1-((1-((1-(4-((2,6-dioxopiperidin-3-yl)carbamoyl)benzoyl)piperidin-4-yl)methyl)piperidin-4-yl)methyl)piperidin-4-yl)-1H-pyrazol-4-yl)pyrazine-2-carboxylate [TDH-539]

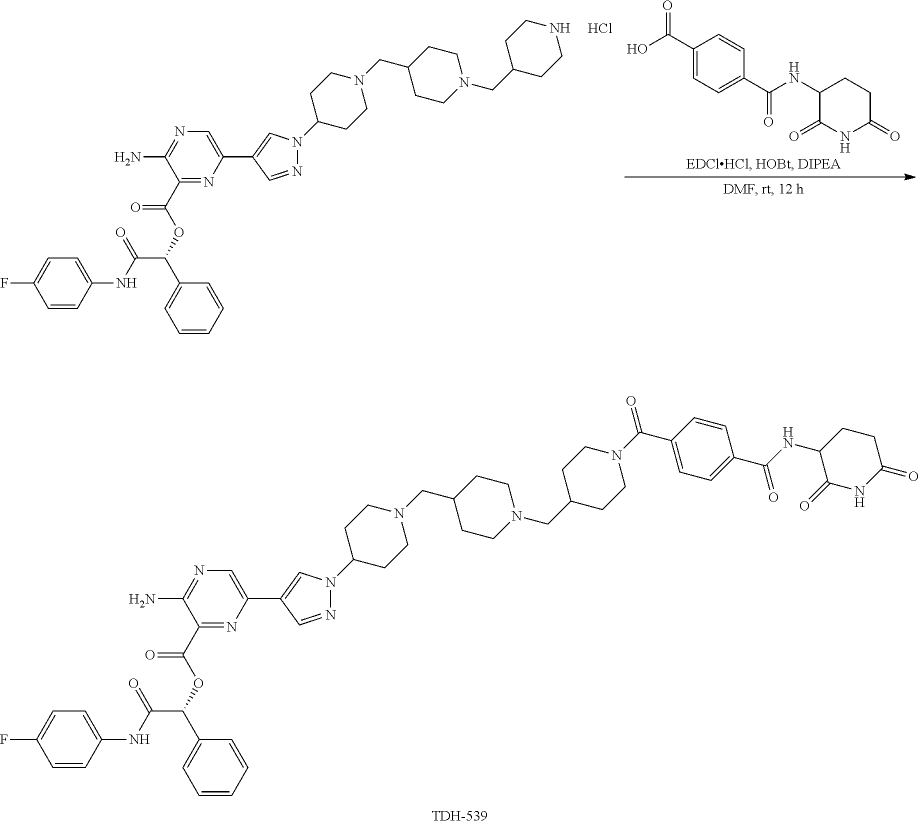 [SMILES: Nc1ncc(-c2cnn(C3CCN(CC4CCN(CC5CCNCC5)CC4)CC3)c2)nc1C(=O)O[C@@H](C(=O)Nc1ccc(F)cc1)c1ccccc1 ; Cl ; O=C1CCC(NC(=O)c2ccc(C(=O)O)cc2)C(=O)N1 ; Nc1ncc(-c2cnn(C3CCN(CC4CCN(CC5CCN(C(=O)c6ccc(C(=O)NC7CCC(=O)NC7=O)cc6)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-((1-(piperidin-4-ylmethyl)piperidin-4-yl)methyl)piperidin-4-yl)-1H-pyrazol-4-yl)pyrazine-2-carboxylate hydrochloride (20 mg, 0.027 mmol, 1 eq.), 4-((2,6-dioxopiperidin-3-yl)carbamoyl)benzoic acid (8 mg, 0.027 mmol, 1.0 eq.), EDCI-HCl (6 mg, 0.030 mmol, 1.1 eq.), HOBt (5 mg, 0.030 mmol, 1.1 eq.), and DIPEA (0.02 mL, 0.14 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure and separated and purified by silica gel column chromatography to obtain (R)-2-((4-fluorophenyl)amino)-2-oxo-1-phenylethyl 3-amino-6-(1-(1-((1-((1-(4-((2,6-dioxopiperidin-3-yl)carbamoyl)benzoyl)piperidin-4-yl)methyl)piperidin-4-yl)methyl)piperidin-4-yl)-1H-pyrazol-4-yl)pyrazine-2-carboxylate (14 mg, 54%).

### Synthesis of (R)-2-((4-fluorophenyl)amino)-2-oxo-1-phenylethyl 3-amino-6-(1-(1-((1-((1-(3-((2,6-dioxopiperidin-3-yl)carbamoyl)benzoyl)piperidin-4-yl)methyl)piperidin-4-yl)methyl)piperidin-4-yl)-1H-pyrazol-4-yl)pyrazine-2-carboxylate [TDH-546]

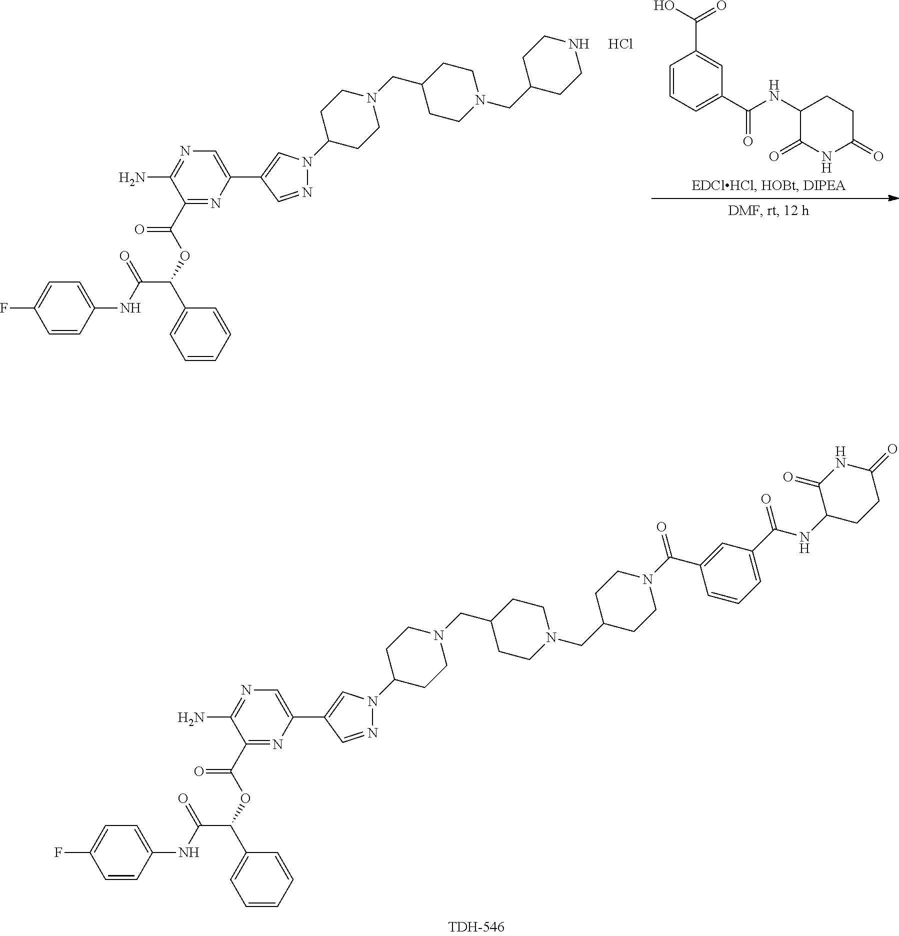 [SMILES: Nc1ncc(-c2cnn(C3CCN(CC4CCN(CC5CCNCC5)CC4)CC3)c2)nc1C(=O)O[C@@H](C(=O)Nc1ccc(F)cc1)c1ccccc1 ; Cl ; O=C1CCC(NC(=O)c2cccc(C(=O)O)c2)C(=O)N1 ; Nc1ncc(-c2cnn(C3CCN(CC4CCN(CC5CCN(C(=O)c6cccc(C(=O)NC7CCC(=O)NC7=O)c6)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-((1-(piperidin-4-ylmethyl)piperidin-4-yl)methyl)piperidin-4-yl)-1H-pyrazol-4-yl)pyrazine-2-carboxylate hydrochloride (20 mg, 0.027 mmol, 1 eq.), 3-((2,6-dioxopiperidin-3-yl)carbamoyl)benzoic acid (8 mg, 0.027 mmol, 1.0 eq.), EDCI-HCl (6 mg, 0.030 mmol, 1.1 eq.), HOBt (5 mg, 0.030 mmol, 1.1 eq.), and DIPEA (0.02 mL, 0.14 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure and separated and purified by silica gel column chromatography to obtain a yellow solid, (R)-2-((4-fluorophenyl)amino)-2-oxo-1-phenylethyl 3-amino-6-(1-(1-((1-((1-(3-((2,6-dioxopiperidin-3-yl)carbamoyl)benzoyl)piperidin-4-yl)methyl)piperidin-4-yl)methyl)piperidin-4-yl)-1H-pyrazol-4-yl)pyrazine-2-carboxylate (12 mg, 46%).

### Synthesis of (R)-2-((4-fluorophenyl)amino)-2-oxo-1-phenylethyl 3-amino-6-(1-(1-((1-((1-(2-(2,6-dioxopiperidin-3-yl)-1-oxoisoindoline-5-carbonyl)piperidin-4-yl)methyl)piperidin-4-yl)methyl)piperidin-4-yl)-1H-pyrazol-4-yl)pyrazine-2-carboxylate [TDH-550]

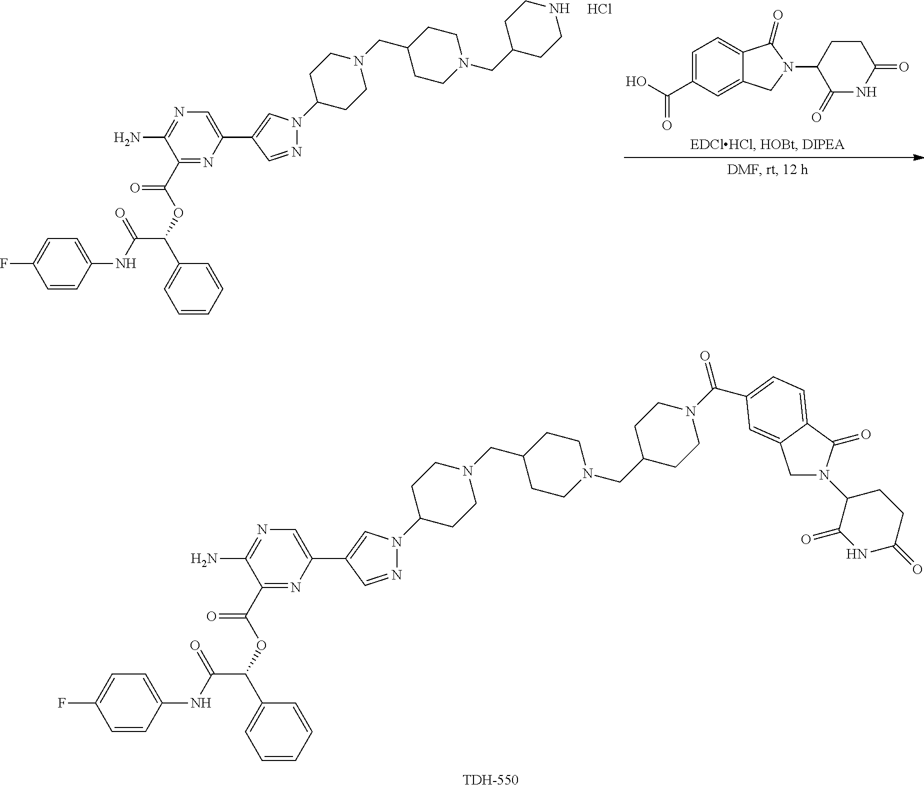 [SMILES: Nc1ncc(-c2cnn(C3CCN(CC4CCN(CC5CCNCC5)CC4)CC3)c2)nc1C(=O)O[C@@H](C(=O)Nc1ccc(F)cc1)c1ccccc1 ; Cl ; O=C1CCC(N2Cc3cc(C(=O)O)ccc3C2=O)C(=O)N1 ; Nc1ncc(-c2cnn(C3CCN(CC4CCN(CC5CCN(C(=O)c6ccc7c(c6)CN(C6CCC(=O)NC6=O)C7=O)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-((1-(piperidin-4-ylmethyl)piperidin-4-yl)methyl)piperidin-4-yl)-1H-pyrazol-4-yl)pyrazine-2-carboxylate hydrochloride (20 mg, 0.027 mmol, 1 eq.), 2-(2,6-dioxopiperidin-3-yl)-1-oxoisoindoline-5-carboxylic acid (8 mg, 0.027 mmol, 1.0 eq.), EDCI-HCl (6 mg, 0.030 mmol, 1.1 eq.), HOBt (5 mg, 0.030 mmol, 1.1 eq.), and DIPEA (0.02 mL, 0.14 mmol, 5 eq.) were dissolved in DMF (1.0 mL) and reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure and separated and purified by silica gel column chromatography to obtain a yellow solid, (R)-2-((4-fluorophenyl)amino)-2-oxo-1-phenylethyl 3-amino-6-(1-(1-((1-((1-(2-(2,6-dioxopiperidin-3-yl)-1-oxoisoindoline-5-carbonyl)piperidin-4-yl)methyl)piperidin-4-yl)methyl)piperidin-4-yl)-1H-pyrazol-4-yl)pyrazine-2-carboxylate (15 mg, 60%).

### Synthesis of (R)-2-((4-fluorophenyl)amino)-2-oxo-1-phenylethyl 3-amino-6-(1-(1-((1-((1-(3-(2,4-dioxotetrahydropyrimidin-1(2H)-yl)benzofuran-6-carbonyl)piperidin-4-yl)methyl)piperidin-4-yl)methyl)piperidin-4-yl)-1H-pyrazol-4-yl)pyrazine-2-carboxylate [TDH-555]

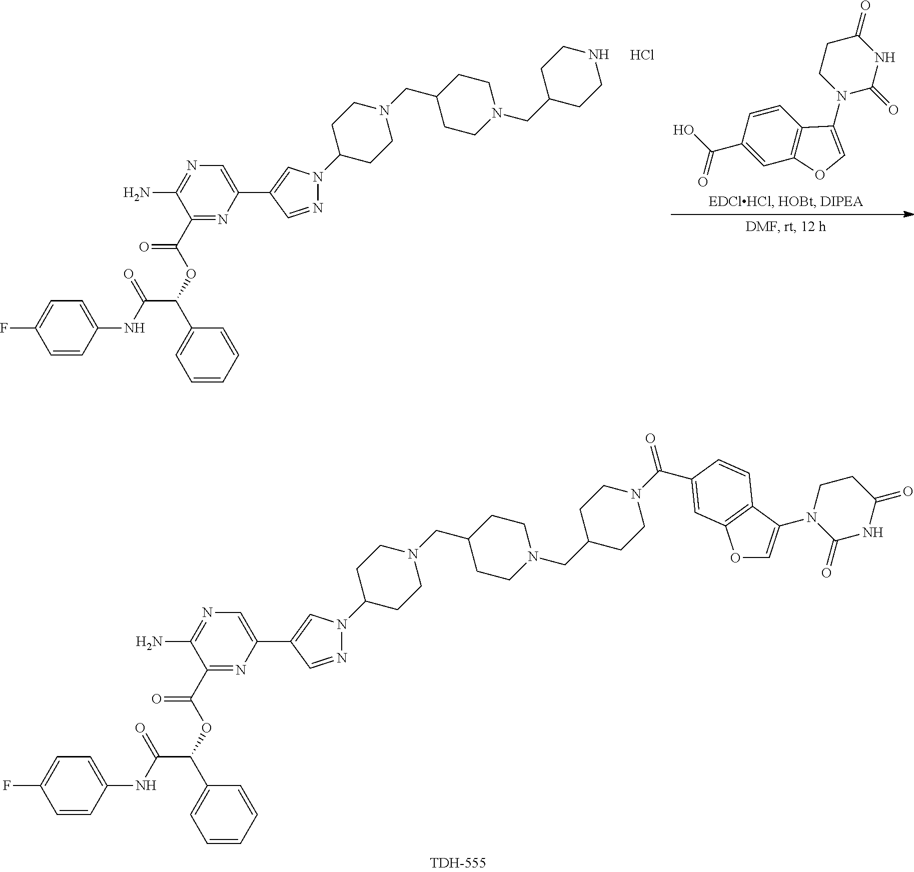 [SMILES: Nc1ncc(-c2cnn(C3CCN(CC4CCN(CC5CCNCC5)CC4)CC3)c2)nc1C(=O)O[C@@H](C(=O)Nc1ccc(F)cc1)c1ccccc1 ; Cl ; O=C1CCN(c2coc3cc(C(=O)O)ccc23)C(=O)N1 ; Nc1ncc(-c2cnn(C3CCN(CC4CCN(CC5CCN(C(=O)c6ccc7c(N8CCC(=O)NC8=O)coc7c6)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-((1-(piperidin-4-ylmethyl)piperidin-4-yl)methyl)piperidin-4-yl)-1H-pyrazol-4-yl)pyrazine-2-carboxylate hydrochloride (20 mg, 0.027 mmol, 1 eq.), 3-(2,4-dioxotetrahydropyrimidin-1(2H)-yl)benzofuran-6-carboxylic acid (8 mg, 0.027 mmol, 1.0 eq.), EDCI-HCl (6 mg, 0.030 mmol, 1.1 eq.), HOBt (5 mg, 0.030 mmol, 1.1 eq.), and DIPEA (0.02 mL, 0.14 mmol, 5 eq.) were dissolved in DMF (1.0 mL) and reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure and separated and purified by silica gel column chromatography to obtain a yellow solid, (R)-2-((4-fluorophenyl)amino)-2-oxo-1-phenylethyl 3-amino-6-(1-(1-((1-((1-(3-(2,4-dioxotetrahydropyrimidin-1(2H)-yl)benzofuran-6-carbonyl)piperidin-4-yl)methyl)piperidin-4-yl)methyl)piperidin-4-yl)-1H-pyrazol-4-yl)pyrazine-2-carboxylate (12 mg, 46%).

### Synthesis of (R)-2-((4-fluorophenyl)amino)-2-oxo-1-phenylethyl 3-amino-6-(1-(1-((1-(2-(1-(3-(2,4-dioxotetrahydropyrimidin-1(2H)-yl)-4-methoxybenzoyl)piperidin-4-yl)ethyl)piperidin-4-yl)methyl)piperidin-4-yl)-1H-pyrazol-4-yl)pyrazine-2-carboxylate [TDH-501]

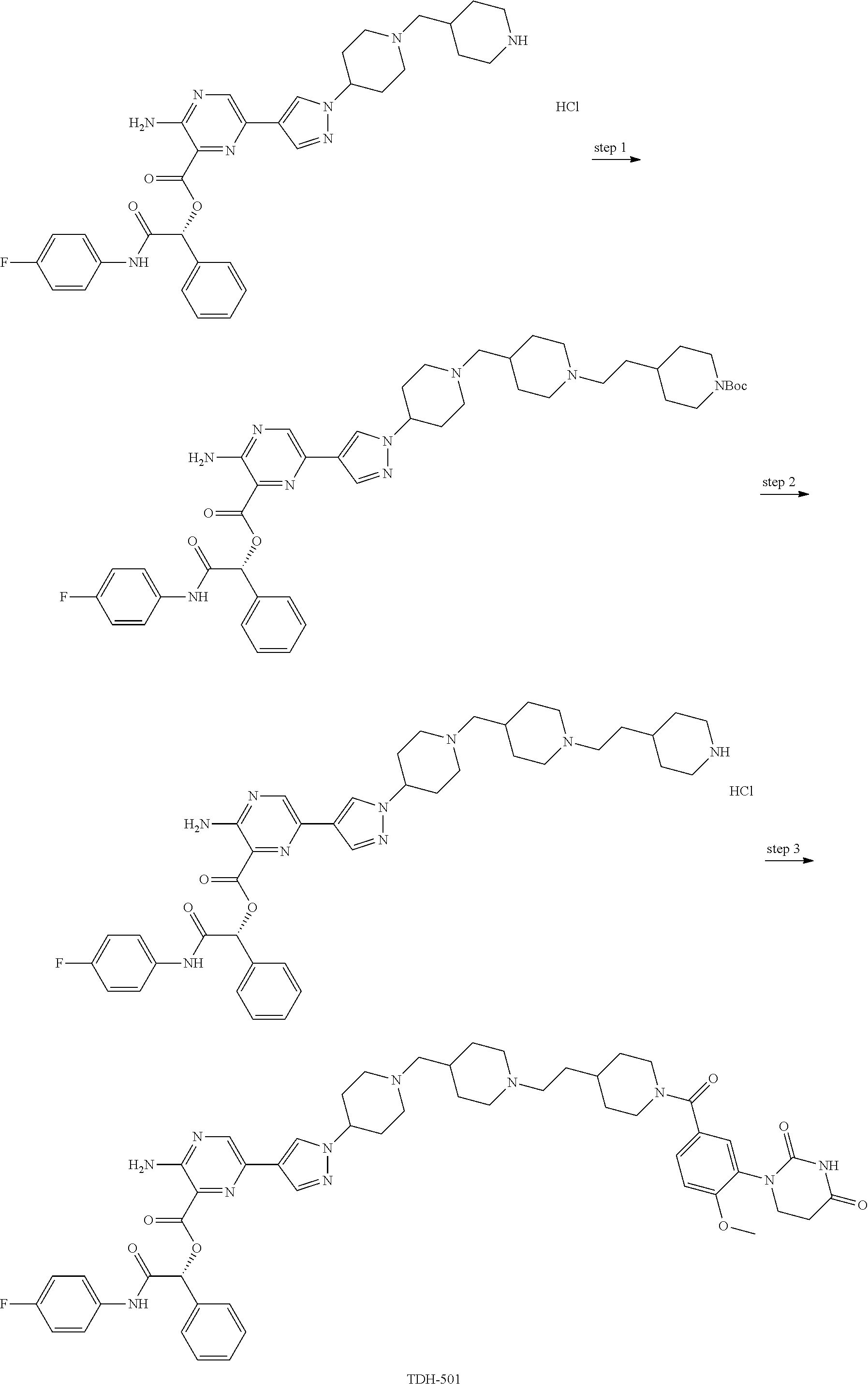 [SMILES: Nc1ncc(-c2cnn(C3CCN(CC4CCNCC4)CC3)c2)nc1C(=O)O[C@@H](C(=O)Nc1ccc(F)cc1)c1ccccc1 ; Cl ; CC(C)(C)OC(=O)N1CCC(CCN2CCC(CN3CCC(n4cc(-c5cnc(N)c(C(=O)O[C@@H](C(=O)Nc6ccc(F)cc6)c6ccccc6)n5)cn4)CC3)CC2)CC1 ; Nc1ncc(-c2cnn(C3CCN(CC4CCN(CCC5CCNCC5)CC4)CC3)c2)nc1C(=O)O[C@@H](C(=O)Nc1ccc(F)cc1)c1ccccc1 ; Cl ; COc1ccc(C(=O)N2CCC(CCN3CCC(CN4CCC(n5cc(-c6cnc(N)c(C(=O)O[C@@H](C(=O)Nc7ccc(F)cc7)c7ccccc7)n6)cn5)CC4)CC3)CC2)cc1N1CCC(=O)NC1=O]

### (R)-2-((4-fluorophenyl)amino)-2-oxo-1-phenylethyl 3-amino-6-(1-(1-((1-(2-(1-(tert-butoxycarbonyl)piperidin-4-yl)ethyl)piperidin-4-yl)methyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (200 mg, 0.31 mmol, 1 eq.) was dissolved in DCM (1.2 mL) and DIPEA (0.27 mL, 1.5 mmol, 5.0 eq.) was added at 0° C. After 10 minutes, tert-butyl 4-(2-oxoethyl)piperidine-1-carboxylate (105 mg, 0.46 mmol, 1.5 eq.) was added to the above solution and reacted at room temperature for 4 hours. After adding NaBH(OAc)₃ (88 mg, 0.41 mmol, 1.34 eq.) at 0° C., the mixture was reacted overnight at room temperature. After completion of the reaction, the mixture was diluted with water and extracted with DCM. The combined organic layer was filtered after removing the residual water through anhydrous Na₂SO₄. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(2-(1-(tert-butoxycarbonyl)piperidin-4-yl)ethyl)piperidin-4-yl)methyl)piperidin-4-yl)-1H-pyrazol-4-yl)pyrazine-2-carboxylate (150 mg, mixture).

### (R)-2-((4-fluorophenyl)amino)-2-oxo-1-phenylethyl 3-amino-6-(1-(1-((1-(2-(piperidin-4-yl)ethyl)piperidin-4-yl)methyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-((1-(2-(1-(tert-butoxycarbonyl)piperidin-4-yl)ethyl)piperidin-4-yl)methyl)piperidin-4-yl)-1H-pyrazol-4-yl)pyrazine-2-carboxylate (150 mg, 0.18 mmol) was dissolved in DCM (2.0 mL). Then, 4 N HCl in dioxane (1.0 mL) was slowly added and reacted at room temperature for 1 hour. After completion of the reaction, the precipitate was filtered to obtain (R)-2-((4-fluorophenyl)amino)-2-oxo-1-phenylethyl 3-amino-6-(1-(1-((1-(2-(piperidin-4-yl)ethyl)piperidin-4-yl)methyl)piperidin-4-yl)-1H-pyrazol-4-yl)pyrazine-2-carboxylate hydrochloride (130 mg, mixture).

### Synthesis of (R)-2-((4-fluorophenyl)amino)-2-oxo-1-phenylethyl 3-amino-6-(1-(1-((1-(2-(1-(3-(2,4-dioxotetrahydropyrimidin-1(2H)-yl)-4-methoxybenzoyl)piperidin-4-yl)ethyl)piperidin-4-yl)methyl)piperidin-4-yl)-1H-pyrazol-4-yl)pyrazine-2-carboxylate [step 3, TDH-501]

(R)-2-((4-fluorophenyl)amino)-2-oxo-1-phenylethyl 3-amino-6-(1-(1-((1-(2-(piperidin-4-yl)ethyl)piperidin-4-yl)methyl)piperidin-4-yl)-1H-pyrazol-4-yl)pyrazine-2-carboxylate hydrochloride (20 mg, 0.026 mmol, 1 eq.), 3-(2,4-dioxotetrahydropyrimidin-1(2H)-yl)-4-methoxybenzoic acid (7 mg, 0.026 mmol, 1.0 eq.), EDCI-HCl (6 mg, 0.029 mmol, 1.1 eq.), HOBt (4 mg, 0.029 mmol, 1.1 eq.) and DIPEA (0.02 mL, 0.13 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(2-(1-(3-(2,4-dioxotetrahydropyrimidin-1(2H)-yl)-4-methoxybenzoyl)piperidin-4-yl)ethyl)piperidin-4-yl)methyl)piperidin-4-yl)-1H-pyrazol-4-yl)pyrazine-2-carboxylate (13 mg, 54%).

### Synthesis of (R)-2-((4-fluorophenyl)amino)-2-oxo-1-phenylethyl 3-amino-6-(1-(1-((1-(2-(1-(1-(3-(2,4-dioxotetrahydropyrimidin-1(2H)-yl)-4-methoxybenzoyl)piperidine-4-carbonyl)piperidin-4-yl)ethyl)piperidin-4-yl)methyl)piperidin-4-yl)-1H-pyrazol-4-yl)pyrazine-2-carboxylate [TDH-502]

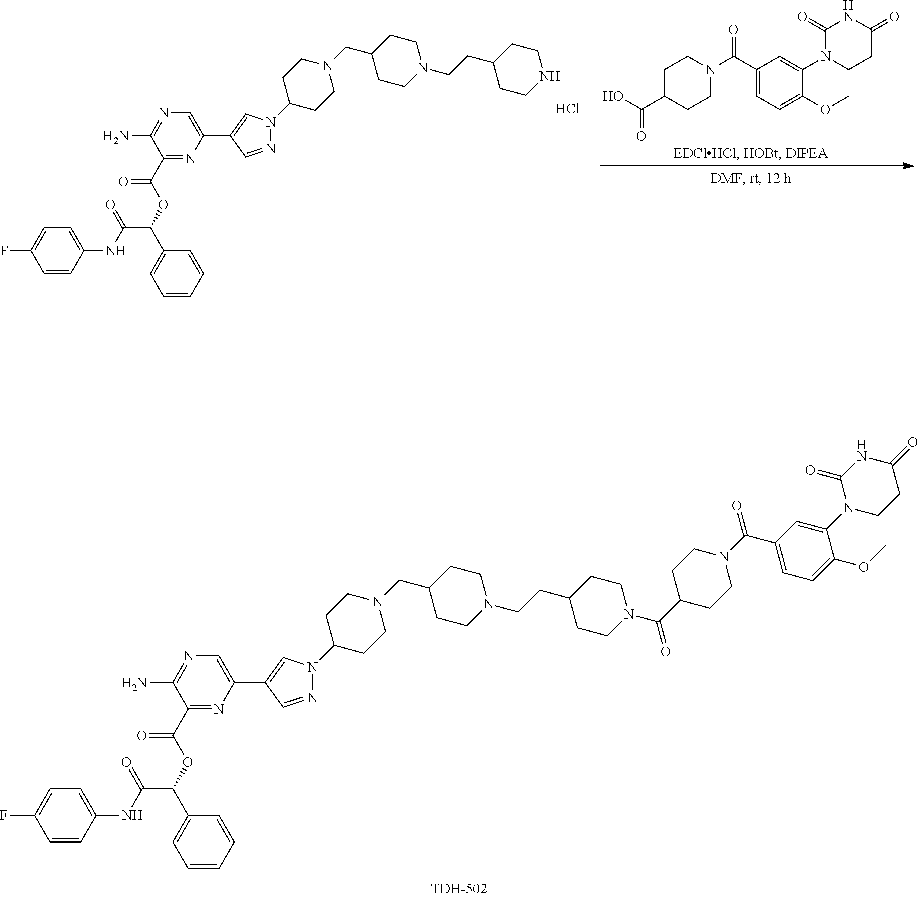 [SMILES: Nc1ncc(-c2cnn(C3CCN(CC4CCN(CCC5CCNCC5)CC4)CC3)c2)nc1C(=O)O[C@@H](C(=O)Nc1ccc(F)cc1)c1ccccc1 ; Cl ; COc1ccc(C(=O)N2CCC(C(=O)O)CC2)cc1N1CCC(=O)NC1=O ; COc1ccc(C(=O)N2CCC(C(=O)N3CCC(CCN4CCC(CN5CCC(n6cc(-c7cnc(N)c(C(=O)O[C@@H](C(=O)Nc8ccc(F)cc8)c8ccccc8)n7)cn6)CC5)CC4)CC3)CC2)cc1N1CCC(=O)NC1=O]

(R)-2-((4-fluorophenyl)amino)-2-oxo-1-phenylethyl 3-amino-6-(1-(1-((1-(2-(piperidin-4-yl)ethyl)piperidin-4-yl)methyl)piperidin-4-yl)-1H-pyrazol-4-yl)pyrazine-2-carboxylate hydrochloride (20 mg, 0.026 mmol, 1 eq.), 1-(3-(2,4-dioxotetrahydropyrimidin-1(2H)-yl)-4-methoxybenzoyl)piperidine-4-carboxylic acid (10 mg, 0.026 mmol, 1.0 eq.), EDCI-HCl (6 mg, 0.029 mmol, 1.1 eq.), HOBt (4 mg, 0.029 mmol, 1.1 eq.) and DIPEA (0.02 mL, 0.13 mmol, 5 eq.) were dissolved in DMF (1.0 mL), and the mixture was reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(2-(1-(1-(3-(2,4-dioxotetrahydropyrimidin-1(2H)-yl)-4-methoxybenzoyl)piperidine-4-carbonyl)piperidin-4-yl)ethyl)piperidin-4-yl)methyl)piperidin-4-yl)-1H-pyrazol-4-yl)pyrazine-2-carboxylate (11 mg, 41%).

### Synthesis of (R)-2-((4-fluorophenyl)amino)-2-oxo-1-phenylethyl 3-amino-6-(1-(1-((1-(2-(1-(2-(3-((1-(2,6-dioxopiperidin-3-yl)-2,5-dioxo-2,5-dihydro-1H-pyrrol-3-yl)amino)phenyl)acetyl)piperidin-4-yl)ethyl)piperidin-4-yl)methyl)piperidin-4-yl)-1H-pyrazol-4-yl)pyrazine-2-carboxylate [TDH-507]

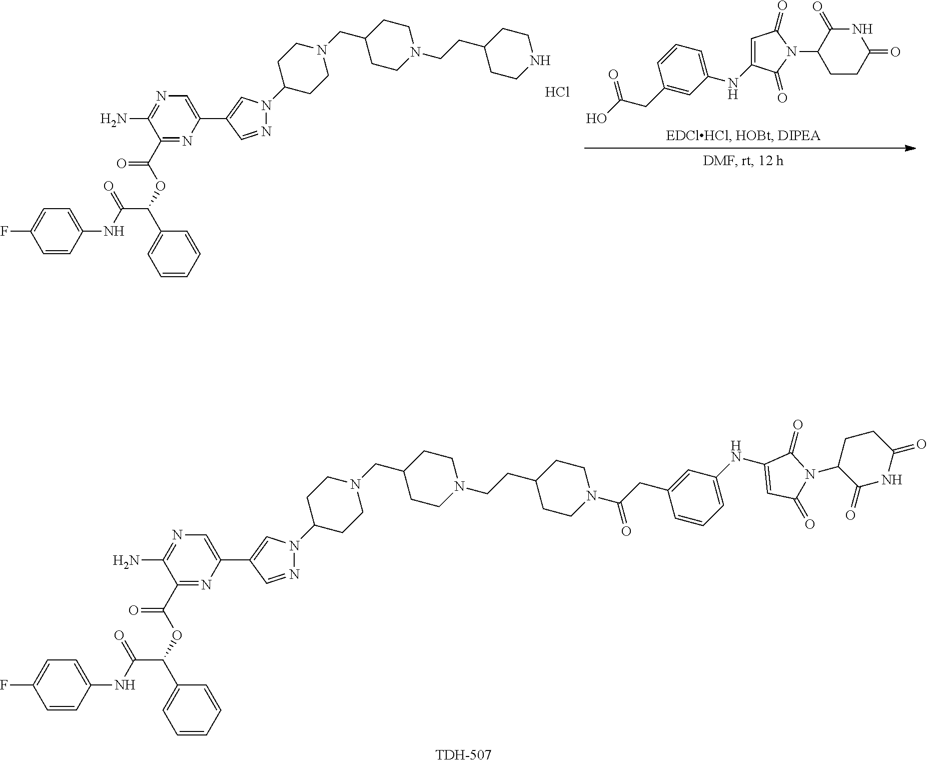 [SMILES: Nc1ncc(-c2cnn(C3CCN(CC4CCN(CCC5CCNCC5)CC4)CC3)c2)nc1C(=O)O[C@@H](C(=O)Nc1ccc(F)cc1)c1ccccc1 ; Cl ; O=C(O)Cc1cccc(NC2=CC(=O)N(C3CCC(=O)NC3=O)C2=O)c1 ; Nc1ncc(-c2cnn(C3CCN(CC4CCN(CCC5CCN(C(=O)Cc6cccc(NC7=CC(=O)N(C8CCC(=O)NC8=O)C7=O)c6)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-((1-(2-(piperidin-4-yl)ethyl)piperidin-4-yl)methyl)piperidin-4-yl)-1H-pyrazol-4-yl)pyrazine-2-carboxylate hydrochloride (20 mg, 0.026 mmol, 1 eq.), 2-(3-((1-(2,6-dioxopiperidin-3-yl)-2,5-dioxo-2,5-dihydro-1H-pyrrol-3-yl)amino)phenyl)acetic acid (10 mg, 0.026 mmol, 1.0 eq.), EDCI-HCl (6 mg, 0.029 mmol, 1.1 eq.), HOBt (4 mg, 0.029 mmol, 1.1 eq.), and DIPEA (0.02 mL, 0.13 mmol, 5 eq.) were dissolved in DMF (1.0 mL), and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(2-(1-(2-(3-((1-(2,6-dioxopiperidin-3-yl)-2,5-dioxo-2,5-dihydro-1H-pyrrol-3-yl)amino)phenyl)acetyl)piperidin-4-yl)ethyl)piperidin-4-yl)methyl)piperidin-4-yl)-1H-pyrazol-4-yl)pyrazine-2-carboxylate (14 mg, 51%).

### Synthesis of (R)-2-((4-fluorophenyl)amino)-2-oxo-1-phenylethyl 3-amino-6-(1-(1-((1-(2-(1-(2-(2,6-dioxopiperidin-3-yl)-6-fluoro-1,3-dioxoisoindolin-5-yl)piperidin-4-yl)ethyl)piperidin-4-yl)methyl)piperidin-4-yl)-1H-pyrazol-4-yl)pyrazine-2-carboxylate [TDH-513]

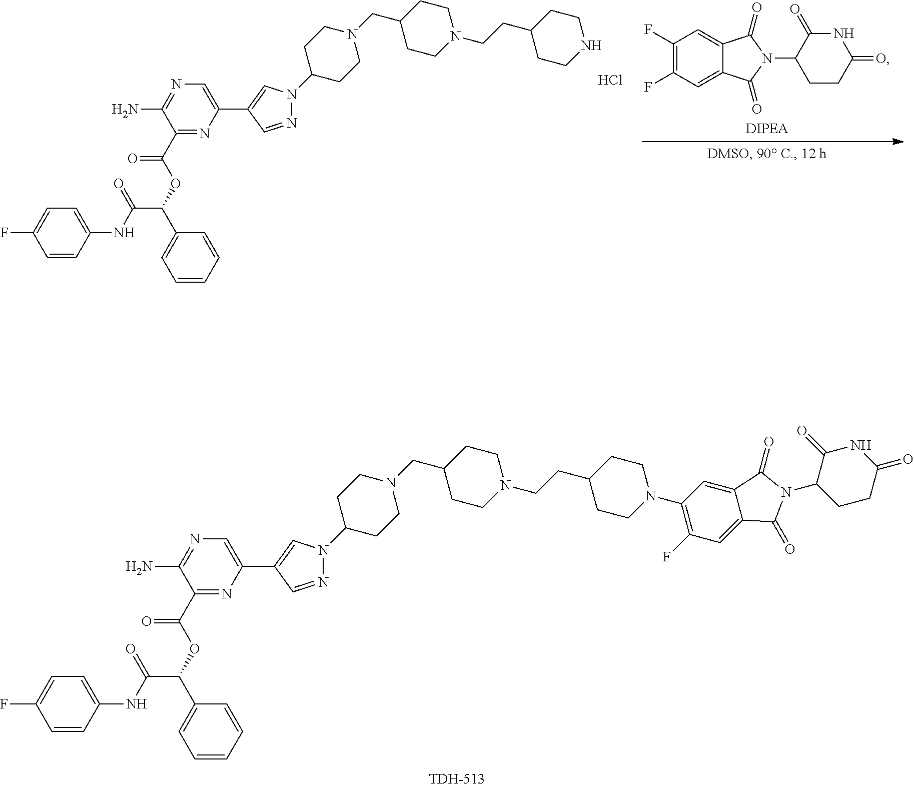

(R)-2-((4-fluorophenyl)amino)-2-oxo-1-phenylethyl 3-amino-6-(1-(1-((1-(2-(piperidin-4-yl)ethyl)piperidin-4-yl)methyl)piperidin-4-yl)-1H-pyrazol-4-yl)pyrazine-2-carboxylate hydrochloride (20 mg, 0.027 mmol, 1 eq.), 2-(2,6-dioxopiperidin-3-yl)-5,6-difluoroisoindoline-1,3-dione (7 mg, 0.025 mmol, 0.95 eq.) and DIPEA (0.014 mL, 0.079 mmol, 3 eq.) were dissolved in DMSO (1.0 mL), and reacted overnight at 90° C. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(2-(1-(2-(2,6-dioxopiperidin-3-yl)-6-fluoro-1,3-dioxoisoindolin-5-yl)piperidin-4-yl)ethyl)piperidin-4-yl)methyl)piperidin-4-yl)-1H-pyrazol-4-yl)pyrazine-2-carboxylate (15 mg, 58%).

### Synthesis of (R)-2-((4-fluorophenyl)amino)-2-oxo-1-phenylethyl 3-amino-6-(1-(1-((1-(7-(3-(2,4-dioxotetrahydropyrimidin-1(2H)-yl)-4-methoxybenzoyl)-7-azaspiro[3.5]nonan-2-yl)piperidin-4-yl)methyl)piperidin-4-yl)-1H-pyrazol-4-yl)pyrazine-2-carboxylate [TDH-503]

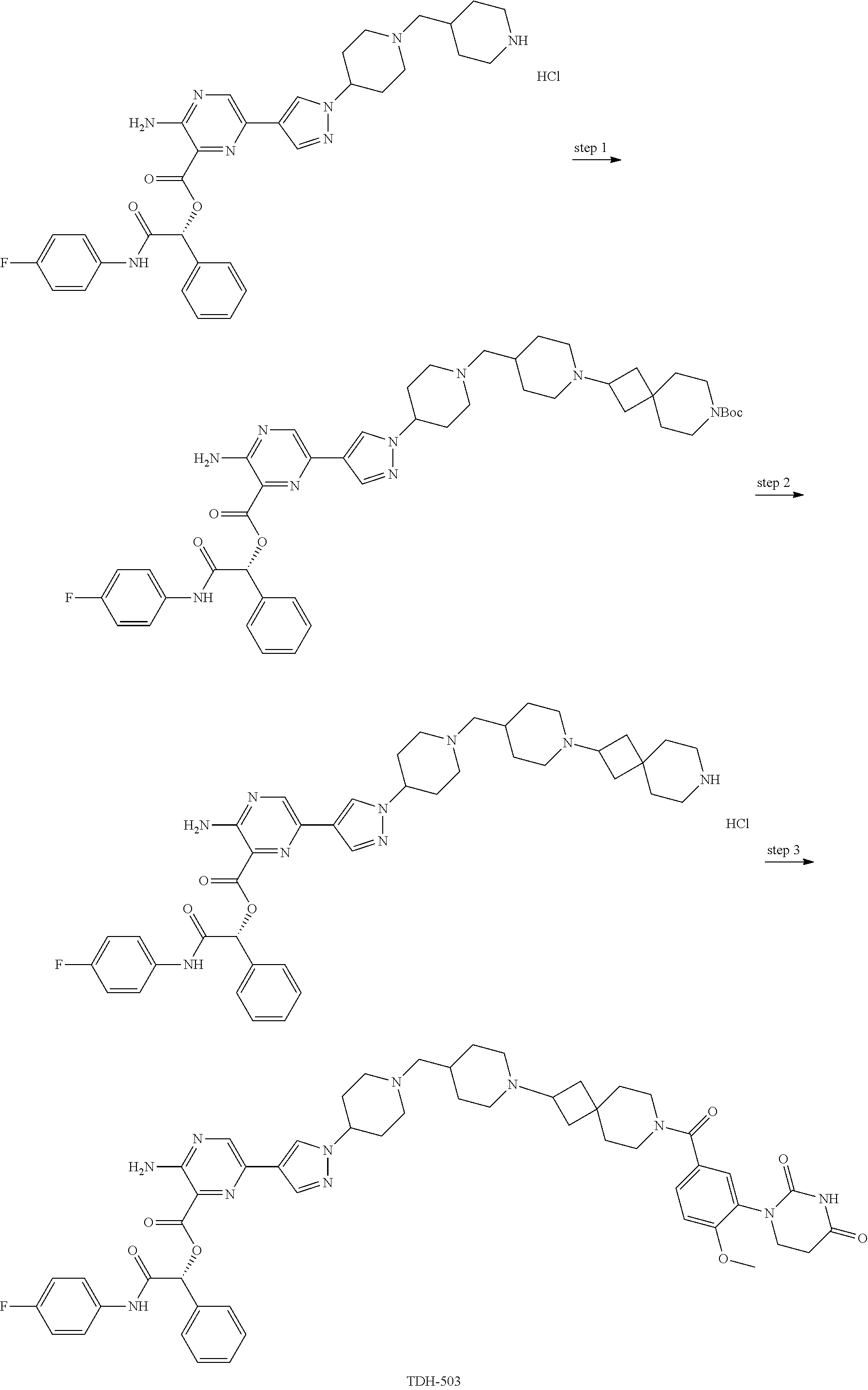 [SMILES: Nc1ncc(-c2cnn(C3CCN(CC4CCNCC4)CC3)c2)nc1C(=O)O[C@@H](C(=O)Nc1ccc(F)cc1)c1ccccc1 ; Cl ; CC(C)(C)OC(=O)N1CCC2(CC1)CC(N1CCC(CN3CCC(n4cc(-c5cnc(N)c(C(=O)O[C@@H](C(=O)Nc6ccc(F)cc6)c6ccccc6)n5)cn4)CC3)CC1)C2 ; Nc1ncc(-c2cnn(C3CCN(CC4CCN(C5CC6(CCNCC6)C5)CC4)CC3)c2)nc1C(=O)O[C@@H](C(=O)Nc1ccc(F)cc1)c1ccccc1 ; Cl ; COc1ccc(C(=O)N2CCC3(CC2)CC(N2CCC(CN4CCC(n5cc(-c6cnc(N)c(C(=O)O[C@@H](C(=O)Nc7ccc(F)cc7)c7ccccc7)n6)cn5)CC4)CC2)C3)cc1N1CCC(=O)NC1=O]

### Tert-butyl (R)-2-(4-((4-(4-(5-amino-6-((2-((4-fluorophenyl)amino)-2-oxo-1-phenylethoxy)carbonyl)pyrazin-2-yl)-1H-pyrazol-1-yl)piperidin-1-yl)methyl)piperidin-1-yl)-7-azaspiro[3.5]nonane-7-carboxylate (step 1)

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (200 mg, 0.31 mmol, 1 eq.) was dissolved in DCM (1.2 mL) and then DIPEA (0.27 mL, 1.5 mmol, 5.0 eq.) was added at 0° C. After 10 minutes, tert-butyl tert-butyl 2-oxo-7-azaspiro[3.5]nonane-7-carboxylate (111 mg, 0.46 mmol, 1.5 eq.) was added to the above solution and reacted at room temperature for 4 hours. After adding NaBH(OAc)₃ (88 mg, 0.41 mmol, 1.34 eq.) at 0° C., the mixture was reacted overnight at room temperature. After completion of the reaction, the mixture was diluted with water and extracted with DCM. The combined organic layer was filtered after removing the residual water through anhydrous Na₂SO₄. The organic layer was concentrated under reduced pressure. The concentrate was separated and purified by silica gel column chromatography to obtain the desired yellow solid, tert-butyl (R)-2-(4-((4-(4-(5-amino-6-((2-((4-fluorophenyl)amino)-2-oxo-1-phenylethoxy)carbonyl)pyrazin-2-yl)-1H-pyrazol-1-yl)piperidin-1-yl)methyl)piperidin-1-yl)-7-azaspiro[3.5]nonane-7-carboxylate (49 mg, mixture).

### (R)-2-((4-fluorophenyl)amino)-2-oxo-1-phenylethyl 6-(1-(1-((1-(7-azaspiro[3.5]nonan-2-yl)piperidin-4-yl)methyl)piperidin-4-yl)-1H-pyrazol-4-yl)-3-aminopyrazine-2-carboxylate hydrochloride (step 2)

Tert-butyl (R)-2-(4-((4-(4-(5-amino-6-((2-((4-fluorophenyl)amino)-2-oxo-1-phenylethoxy)carbonyl)pyrazin-2-yl)-1H-pyrazol-1-yl)piperidin-1-yl)methyl)piperidin-1-yl)-7-azaspiro[3.5]nonane-7-carboxylate (49 mg, 0.09 mmol) was dissolved in DCM (1.0 mL). Then, 4 N HCl in dioxane (0.5 mL) was slowly added, and reacted at room temperature for 1 hour. After completion of the reaction, the precipitate was filtered to obtain (R)-2-((4-fluorophenyl)amino)-2-oxo-1-phenylethyl 6-(1-(1-((1-(7-azaspiro[3.5]nonan-2-yl)piperidin-4-yl)methyl)piperidin-4-yl)-1H-pyrazol-4-yl)-3-aminopyrazine-2-carboxylate hydrochloride (34 mg, mixture).

### Synthesis of (R)-2-((4-fluorophenyl)amino)-2-oxo-1-phenylethyl 3-amino-6-(1-(1-((1-(7-(3-(2,4-dioxotetrahydropyrimidin-1(2H)-yl)-4-methoxybenzoyl)-7-azaspiro[3.5]nonan-2-yl)piperidin-4-yl)methyl)piperidin-4-yl)-1H-pyrazol-4-yl)pyrazine-2-carboxylate [step 3, TDH-503]

(R)-2-((4-fluorophenyl)amino)-2-oxo-1-phenylethyl 6-(1-(1-((1-(7-azaspiro[3.5]nonan-2-yl)piperidin-4-yl)methyl)piperidin-4-yl)-1H-pyrazol-4-yl)-3-aminopyrazine-2-carboxylate hydrochloride (20 mg, 0.026 mmol, 1 eq.), 3-(2,4-dioxotetrahydropyrimidin-1(2H)-yl)-4-methoxybenzoic acid (7 mg, 0.026 mmol, 1.0 eq.), EDCI-HCl (6 mg, 0.029 mmol, 1.1 eq.), HOBt (4 mg, 0.029 mmol, 1.1 eq.) and DIPEA (0.02 mL, 0.13 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(7-(3-(2,4-dioxotetrahydropyrimidin-1(2H)-yl)-4-methoxybenzoyl)-7-azaspiro[3.5]nonan-2-yl)piperidin-4-yl)methyl)piperidin-4-yl)-1H-pyrazol-4-yl)pyrazine-2-carboxylate (11 mg, 43%).

### Synthesis of (R)-2-((4-fluorophenyl)amino)-2-oxo-1-phenylethyl 3-amino-6-(1-(1-((1-(7-(1-(3-(2,4-dioxotetrahydropyrimidin-1(2H)-yl)-4-methoxybenzoyl)piperidine-4-carbonyl)-7-azaspiro[3.5]nonan-2-yl)piperidin-4-yl)methyl)piperidin-4-yl)-1H-pyrazol-4-yl)pyrazine-2-carboxylate (TDH-508)

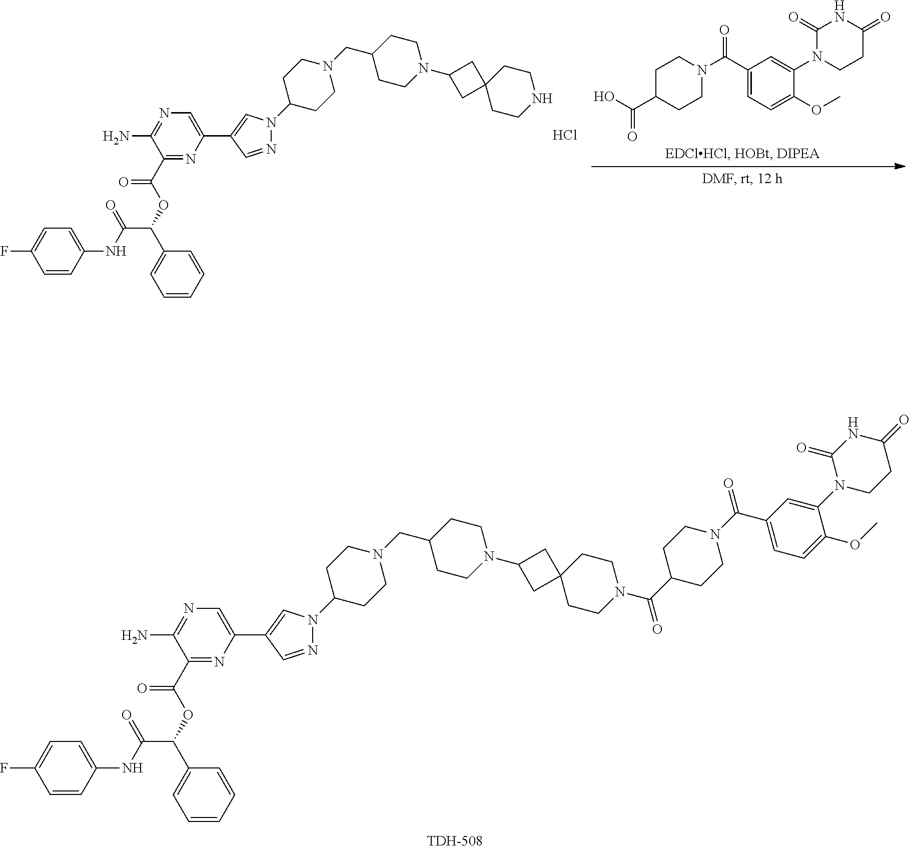 [SMILES: Nc1ncc(-c2cnn(C3CCN(CC4CCN(C5CC6(CCNCC6)C5)CC4)CC3)c2)nc1C(=O)O[C@@H](C(=O)Nc1ccc(F)cc1)c1ccccc1 ; Cl ; COc1ccc(C(=O)N2CCC(C(=O)O)CC2)cc1N1CCC(=O)NC1=O ; COc1ccc(C(=O)N2CCC(C(=O)N3CCC4(CC3)CC(N3CCC(CN5CCC(n6cc(-c7cnc(N)c(C(=O)O[C@@H](C(=O)Nc8ccc(F)cc8)c8ccccc8)n7)cn6)CC5)CC3)C4)CC2)cc1N1CCC(=O)NC1=O]

(R)-2-((4-fluorophenyl)amino)-2-oxo-1-phenylethyl 6-(1-(1-((1-(7-azaspiro[3.5]nonan-2-yl)piperidin-4-yl)methyl)piperidin-4-yl)-1H-pyrazol-4-yl)-3-aminopyrazine-2-carboxylate hydrochloride (15 mg, 0.016 mmol, 1 eq.), 1-(3-(2,4-dioxotetrahydropyrimidin-1(2H)-yl)-4-methoxybenzoyl)piperidine-4-carboxylic acid (6 mg, 0.016 mmol, 1.0 eq.), EDCI-HCl (3 mg, 0.018 mmol, 1.1 eq.), HOBt (3 mg, 0.018 mmol, 1.1 eq.) and DIPEA (0.01 mL, 0.08 mmol, 5 eq.) were dissolved in DMF (1.0 mL), and the mixture was reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(7-(1-(3-(2,4-dioxotetrahydropyrimidin-1(2H)-yl)-4-methoxybenzoyl)piperidine-4-carbonyl)-7-azaspiro[3.5]nonan-2-yl)piperidin-4-yl)methyl)piperidin-4-yl)-1H-pyrazol-4-yl)pyrazine-2-carboxylate (8 mg, 49%).

### Synthesis of (R)-2-((4-fluorophenyl)amino)-2-oxo-1-phenylethyl 3-amino-6-(1-(1-((1-(2-((1-(3-(2,4-dioxotetrahydropyrimidin-1(2H)-yl)-4-methoxybenzoyl)piperidin-4-yl)oxy)ethyl)piperidin-4-yl)methyl)piperidin-4-yl)-1H-pyrazol-4-yl)pyrazine-2-carboxylate [TDH-506]

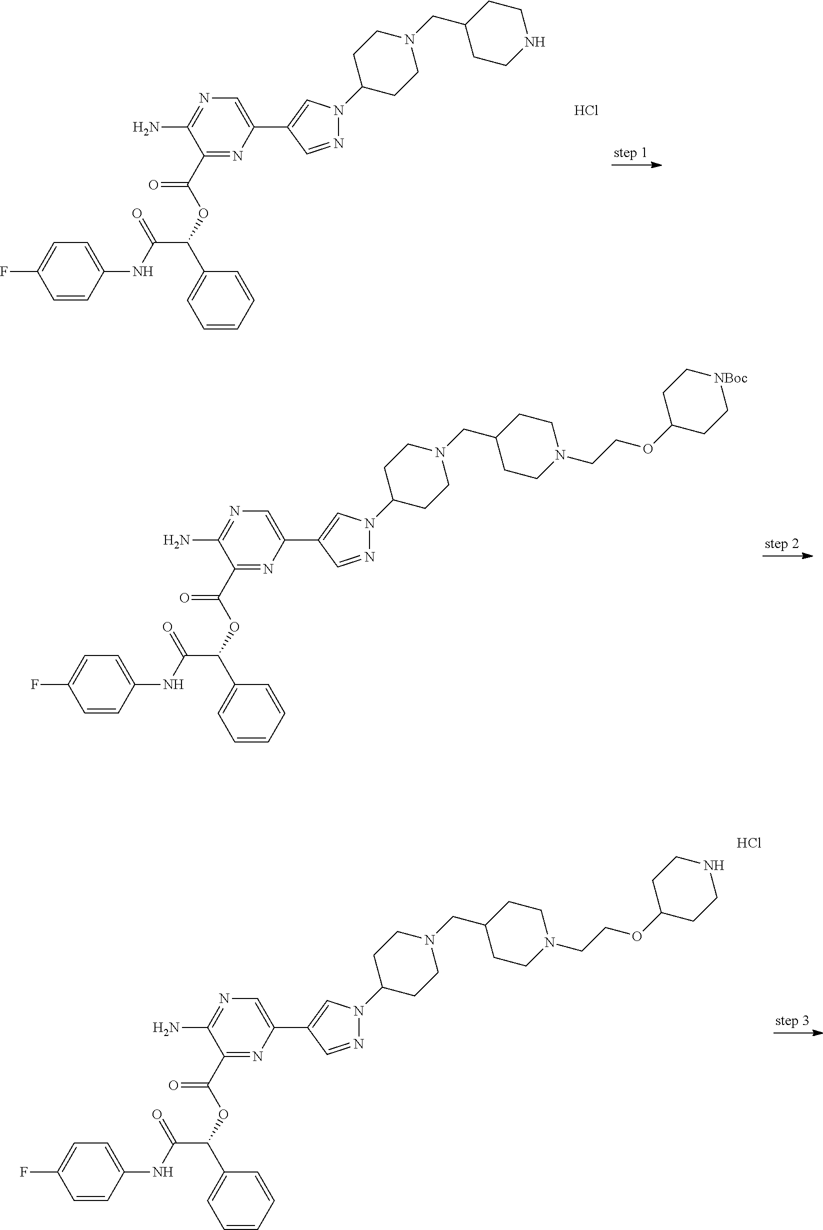 [SMILES: Nc1ncc(-c2cnn(C3CCN(CC4CCNCC4)CC3)c2)nc1C(=O)O[C@@H](C(=O)Nc1ccc(F)cc1)c1ccccc1 ; Cl ; CC(C)(C)OC(=O)N1CCC(OCCN2CCC(CN3CCC(n4cc(-c5cnc(N)c(C(=O)O[C@@H](C(=O)Nc6ccc(F)cc6)c6ccccc6)n5)cn4)CC3)CC2)CC1 ; Cl ; Nc1ncc(-c2cnn(C3CCN(CC4CCN(CCOC5CCNCC5)CC4)CC3)c2)nc1C(=O)O[C@@H](C(=O)Nc1ccc(F)cc1)c1ccccc1]

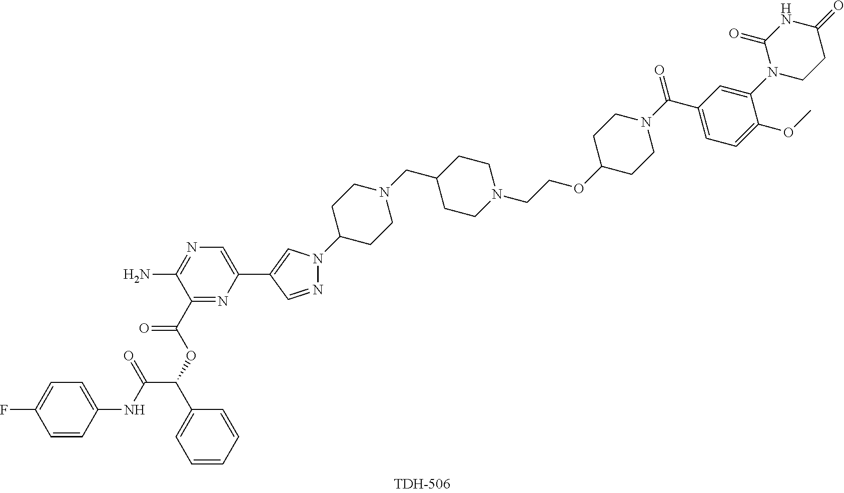 [SMILES: COc1ccc(C(=O)N2CCC(OCCN3CCC(CN4CCC(n5cc(-c6cnc(N)c(C(=O)O[C@@H](C(=O)Nc7ccc(F)cc7)c7ccccc7)n6)cn5)CC4)CC3)CC2)cc1N1CCC(=O)NC1=O]

### Synthesis of (R)-2-((4-fluorophenyl)amino)-2-oxo-1-phenylethyl 3-amino-6-(1-(1-((1-(2-((1-(tert-butoxycarbonyl)piperidin-4-yl)oxy)ethyl)piperidin-4-yl)methyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(1-(piperidin-4-ylmethyl)piperidin-4-yl)-1H-pyrazol-4-yl)pyrazine-2-carboxylate hydrochloride (200 mg, 0.31 mmol, 1.0 eq.), tert-butyl 4-(2-iodoethoxy)piperidine-1-carboxylate (164 mg, 0.46 mmol, 1.5 eq.), and K₂CO₃ (86 mg, 0.62 mmol, 2.0 eq.) were dissolved in DMF (1.2 mL) and reacted at 50° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(2-((1-(tert-butoxycarbonyl)piperidin-4-yl)oxy)ethyl)piperidin-4-yl)methyl)piperidin-4-yl)-1H-pyrazol-4-yl)pyrazine-2-carboxylate (33 mg, mixture).

### Synthesis of (R)-2-((4-fluorophenyl)amino)-2-oxo-1-phenylethyl 3-amino-6-(1-(1-((1-(2-(piperidin-4-yloxy)ethyl)piperidin-4-yl)methyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-((1-(2-((1-(tert-butoxycarbonyl)piperidin-4-yl)oxy)ethyl)piperidin-4-yl)methyl)piperidin-4-yl)-1H-pyrazol-4-yl)pyrazine-2-carboxylate (33 mg, 0.04 mmol) was dissolved in DCM (1.0 mL). Then, 4 N HCl in dioxane (0.5 mL) was slowly added, and reacted at room temperature for 1 hour. After completion of the reaction, the precipitate was filtered to obtain (R)-2-((4-fluorophenyl)amino)-2-oxo-1-phenylethyl 3-amino-6-(1-(1-((1-(2-(piperidin-4-yloxy)ethyl)piperidin-4-yl)methyl)piperidin-4-yl)-1H-pyrazol-4-yl)pyrazine-2-carboxylate hydrochloride (32 mg, mixture).

### Synthesis of (R)-2-((4-fluorophenyl)amino)-2-oxo-1-phenylethyl 3-amino-6-(1-(1-((1-(2-((1-(3-(2,4-dioxotetrahydropyrimidin-1(2H)-yl)-4-methoxybenzoyl)piperidin-4-yl)oxy)ethyl)piperidin-4-yl)methyl)piperidin-4-yl)-1H-pyrazol-4-yl)pyrazine-2-carboxylate [step 3, TDH-506]

(R)-2-((4-fluorophenyl)amino)-2-oxo-1-phenylethyl 3-amino-6-(1-(1-((1-(2-(piperidin-4-yloxy)ethyl)piperidin-4-yl)methyl)piperidin-4-yl)-1H-pyrazol-4-yl)pyrazine-2-carboxylate hydrochloride (20 mg, 0.026 mmol, 1 eq.), 3-(2,4-dioxotetrahydropyrimidin-1(2H)-yl)-4-methoxybenzoic acid (7 mg, 0.026 mmol, 1.0 eq.), EDCI-HCl (6 mg, 0.029 mmol, 1.1 eq.), HOBt (4 mg, 0.029 mmol, 1.1 eq.), and DIPEA (0.02 mL, 0.13 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(2-((1-(3-(2,4-dioxotetrahydropyrimidin-1(2H)-yl)-4-methoxybenzoyl)piperidin-4-yl)oxy)ethyl)piperidin-4-yl)methyl)piperidin-4-yl)-1H-pyrazol-4-yl)pyrazine-2-carboxylate (10 mg, 39%).

### Synthesis of (R)-2-((4-fluorophenyl)amino)-2-oxo-1-phenylethyl 3-amino-6-(1-(1-((1-(2-((1-(1-(3-(2,4-dioxotetrahydropyrimidin-1(2H)-yl)-4-methoxybenzoyl)piperidine-4-carbonyl)piperidin-4-yl)oxy)ethyl)piperidin-4-yl)methyl)piperidin-4-yl)-1H-pyrazol-4-yl)pyrazine-2-carboxylate (TDH-512)

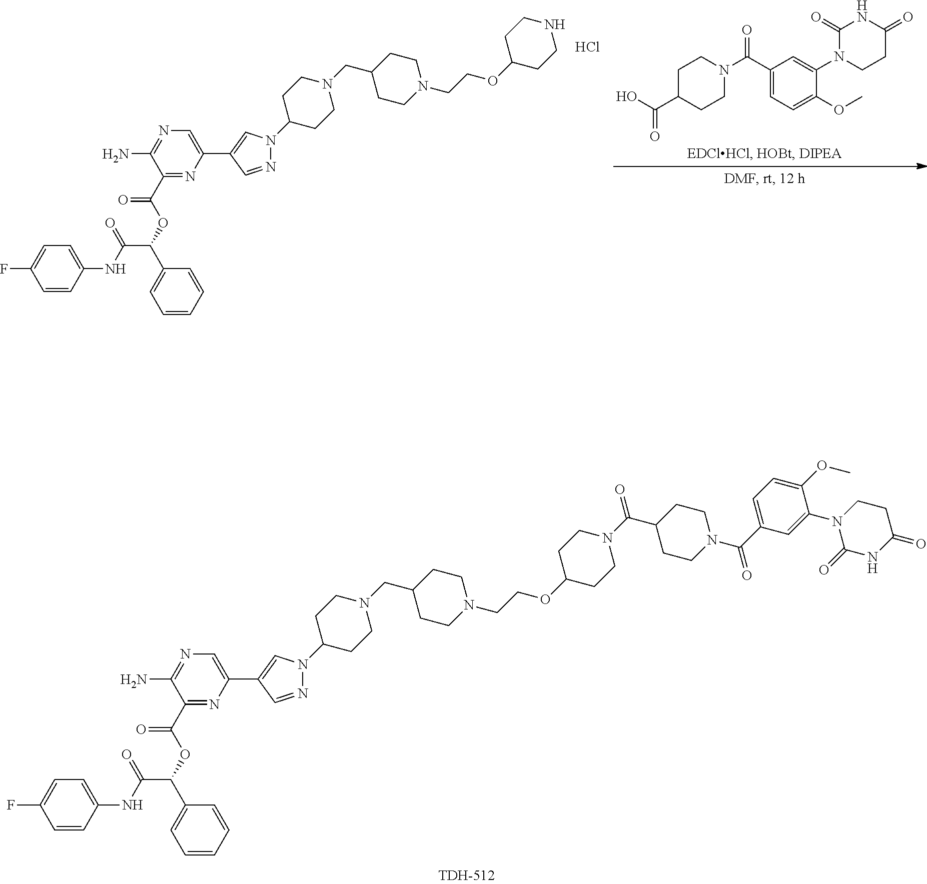 [SMILES: Nc1ncc(-c2cnn(C3CCN(CC4CCN(CCOC5CCNCC5)CC4)CC3)c2)nc1C(=O)O[C@@H](C(=O)Nc1ccc(F)cc1)c1ccccc1 ; Cl ; COc1ccc(C(=O)N2CCC(C(=O)O)CC2)cc1N1CCC(=O)NC1=O ; COc1ccc(C(=O)N2CCC(C(=O)N3CCC(OCCN4CCC(CN5CCC(n6cc(-c7cnc(N)c(C(=O)O[C@@H](C(=O)Nc8ccc(F)cc8)c8ccccc8)n7)cn6)CC5)CC4)CC3)CC2)cc1N1CCC(=O)NC1=O]

(R)-2-((4-fluorophenyl)amino)-2-oxo-1-phenylethyl 3-amino-6-(1-(1-((1-(2-(piperidin-4-yloxy)ethyl)piperidin-4-yl)methyl)piperidin-4-yl)-1H-pyrazol-4-yl)pyrazine-2-carboxylate hydrochloride (12 mg, 0.016 mmol, 1 eq.), 1-(3-(2,4-dioxotetrahydropyrimidin-1(2H)-yl)-4-methoxybenzoyl)piperidine-4-carboxylic acid (6 mg, 0.016 mmol, 1.0 eq.), EDCI-HCl (3 mg, 0.018 mmol, 1.1 eq.), HOBt (3 mg, 0.018 mmol, 1.1 eq.) and DIPEA (0.01 mL, 0.08 mmol, 5 eq.) were dissolved in DMF (1.0 mL), and the mixture was reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-((1-(2-((1-(3-(2,4-dioxotetrahydropyrimidin-1(2H)-yl)-4-methoxybenzoyl)piperidin-4-yl)oxy)ethyl)piperidin-4-yl)methyl)piperidin-4-yl)-1H-pyrazol-4-yl)pyrazine-2-carboxylate (6 mg, 34%).

### Synthesis of (R)-2-((4-fluorophenyl)amino)-2-oxo-1-phenylethyl 3-amino-6-(1-(1-(3-(4-(3-(2,4-dioxotetrahydropyrimidin-1(2H)-yl)-4-methoxybenzoyl)piperazin-1-yl)propyl)piperidin-4-yl)-1H-pyrazol-4-yl)pyrazine-2-carboxylate [TDH-447]

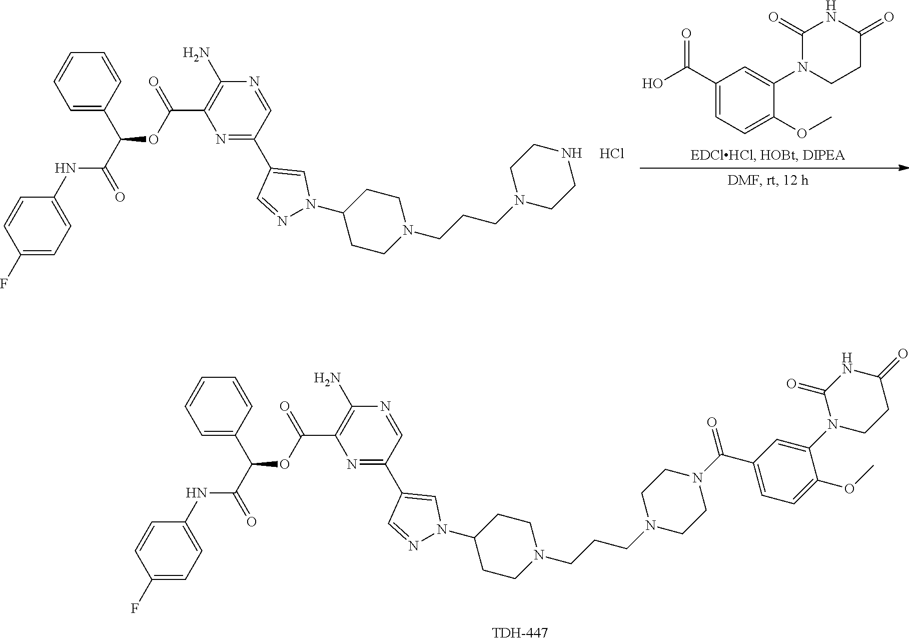 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; COc1ccc(C(=O)O)cc1N1CCC(=O)NC1=O ; COc1ccc(C(=O)N2CCN(CCCN3CCC(n4cc(-c5cnc(N)c(C(=O)O[C@@H](C(=O)Nc6ccc(F)cc6)c6ccccc6)n5)cn4)CC3)CC2)cc1N1CCC(=O)NC1=O]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (9 mg, 0.014 mmol, 1 eq.), 3-(2,4-dioxotetrahydropyrimidin-1(2H)-yl)-4-methoxybenzoic acid (4 mg, 0.014 mmol, 1.0 eq.), EDCI-HCl (3 mg, 0.015 mmol, 1.1 eq.), HOBt (2 mg, 0.015 mmol, 1.1 eq.), and DIPEA (0.01 mL, 0.07 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-(3-(2,4-dioxotetrahydropyrimidin-1(2H)-yl)-4-methoxybenzoyl)piperazin-1-yl)propyl)piperidin-4-yl)-1H-pyrazol-4-yl)pyrazine-2-carboxylate (8 mg, 72%).

### Synthesis of (R)-2-((4-fluorophenyl)amino)-2-oxo-1-phenylethyl 3-amino-6-(1-(1-(3-(4-(3-(2-(2,6-dioxopiperidin-3-yl)-1-oxoisoindolin-5-yl)propanoyl)piperazin-1-yl)propyl)piperidin-4-yl)-1H-pyrazol-4-yl)pyrazine-2-carboxylate [TDH-476]

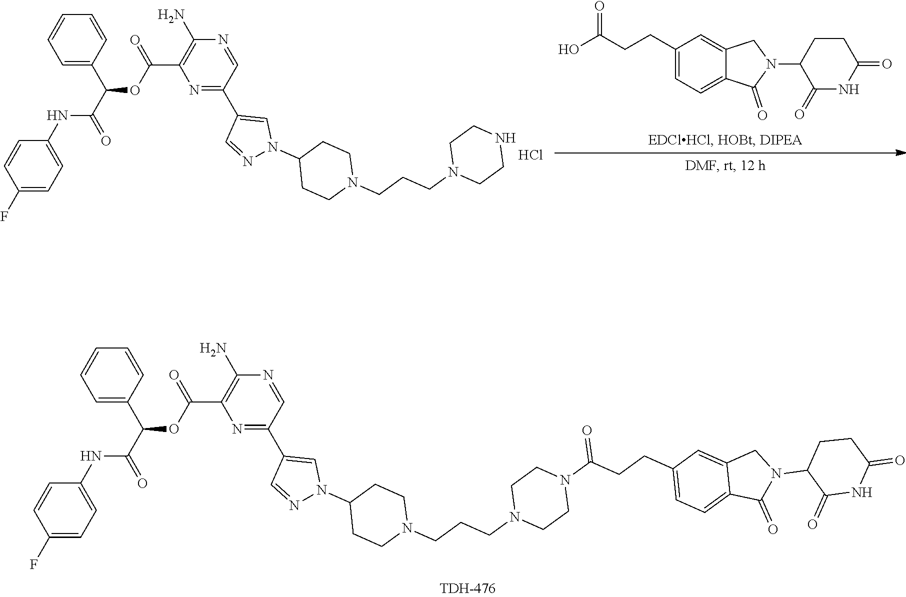 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; O=C(O)CCc1ccc2c(c1)CN(C1CCC(=O)NC1=O)C2=O ; Nc1ncc(-c2cnn(C3CCN(CCCN4CCN(C(=O)CCc5ccc6c(c5)CN(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (20 mg, 0.030 mmol, 1 eq.), 3-(2-(2,6-dioxopiperidin-3-yl)-1-oxoisoindolin-5-yl)propanoic acid (10 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-(3-(2-(2,6-dioxopiperidin-3-yl)-1-oxoisoindolin-5-yl)propanoyl)piperazin-1-yl)propyl)piperidin-4-yl)-1H-pyrazol-4-yl)pyrazine-2-carboxylate (12 mg, 43%).

### Synthesis of 3-amino-6-(1-(1-(3-(4-(2-(2,6-dioxopiperidin-3-yl)-1,3-dioxoisoindolin-5-yl)piperazin-1-yl)propyl)piperidin-4-yl)-1H-pyrazol-4-yl)-N—((R)-2-((4-fluorophenyl)amino)-2-oxo-1-phenylethyl)pyrazine-2-carboxamide [TDH-491]

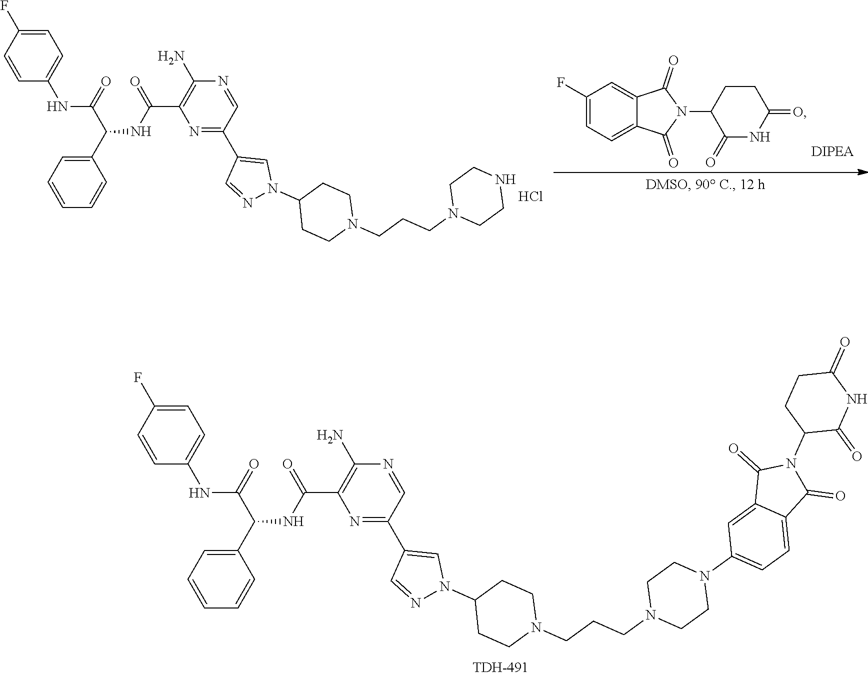 [SMILES: Nc1ncc(-c2cnn(C3CCN(CCCN4CCNCC4)CC3)c2)nc1C(=O)N[C@@H](C(=O)Nc1ccc(F)cc1)c1ccccc1 ; Cl ; O=C1CCC(N2C(=O)c3ccc(F)cc3C2=O)C(=O)N1 ; Nc1ncc(-c2cnn(C3CCN(CCCN4CCN(c5ccc6c(c5)C(=O)N(C5CCC(=O)NC5=O)C6=O)CC4)CC3)c2)nc1C(=O)N[C@@H](C(=O)Nc1ccc(F)cc1)c1ccccc1]

(R)-3-amino-N-(2-((4-fluorophenyl)amino)-2-oxo-1-phenylethyl)-6-(1-(1-(3-(piperazin-1-yl)propyl)piperidin-4-yl)-1H-pyrazol-4-yl)pyrazine-2-carboxamide hydrochloride (20 mg, 0.030 mmol, 1 eq.), 2-(2,6-dioxopiperidin-3-yl)-5-fluoroisoindoline-1,3-dione (8 mg, 0.030 mmol, 1 eq.) and DIPEA (0.015 mL, 0.089 mmol, 3 eq.) were dissolved in DMSO (1.0 mL), and the mixture was reacted at 90° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, 3-amino-6-(1-(1-(3-(4-(2-(2,6-dioxopiperidin-3-yl)-1,3-dioxoisoindolin-5-yl)piperazin-1-yl)propyl)piperidin-4-yl)-1H-pyrazol-4-yl)-N—((R)-2-((4-fluorophenyl)amino)-2-oxo-1-phenylethyl)pyrazine-2-carboxamide (11 mg, 38%).

### Synthesis of (R)-2-((4-fluorophenyl)amino)-2-oxo-1-phenylethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate [TDH-492]

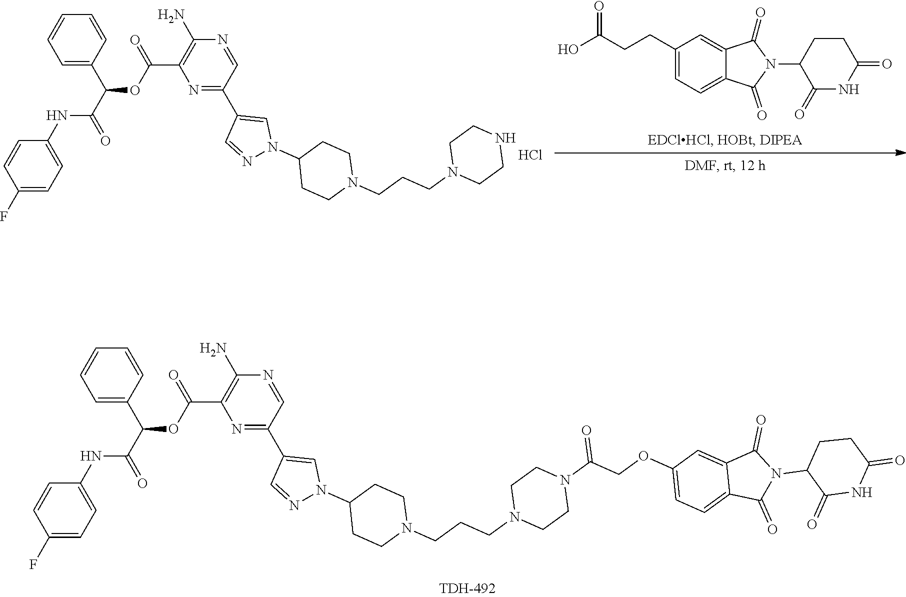 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; O=C(O)CCc1ccc2c(c1)C(=O)N(C1CCC(=O)NC1=O)C2=O ; Nc1ncc(-c2cnn(C3CCN(CCCN4CCN(C(=O)COc5ccc6c(c5)C(=O)N(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (20 mg, 0.030 mmol, 1 eq.), 2-((2-(2,6-dioxopiperidin-3-yl)-1,3-dioxoisoindolin-5-yl)oxy)acetic acid (10 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL), and the mixture was reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate (21 mg, 73%).

### Synthesis of (R)-2-((4-fluorophenyl)amino)-2-oxo-1-phenylethyl 3-amino-6-(1-(1-(3-(4-((2-(2,6-dioxopiperidin-3-yl)-1,3-dioxoisoindolin-5-yl)glycyl)piperazin-1-yl)propyl)piperidin-4-yl)-1H-pyrazol-4-yl)pyrazine-2-carboxylate [TDH-493]

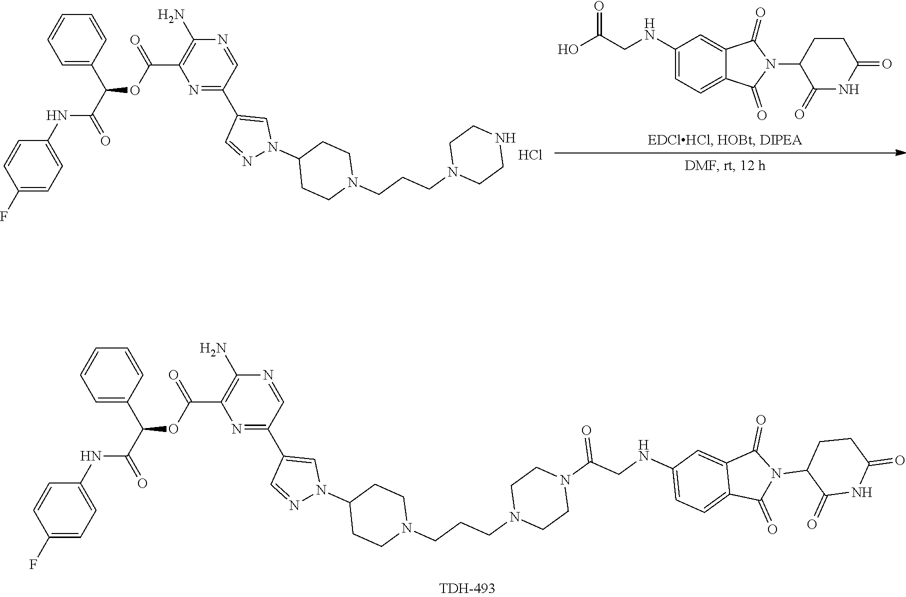 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; O=C(O)CNc1ccc2c(c1)C(=O)N(C1CCC(=O)NC1=O)C2=O ; Nc1ncc(-c2cnn(C3CCN(CCCN4CCN(C(=O)CNc5ccc6c(c5)C(=O)N(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (20 mg, 0.030 mmol, 1 eq.), (2-(2,6-dioxopiperidin-3-yl)-1,3-dioxoisoindolin-5-yl)glycine (10 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL) and reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-(2-((2-(2,6-dioxopiperidin-3-yl)-1-oxoisoindolin-5-yl)oxy)acetyl)piperazin-1-yl)propyl)piperidin-4-yl)-1H-pyrazol-4-yl)pyrazine-2-carboxylate (15 mg, 52%).

### Synthesis of (R)-2-((4-fluorophenyl)amino)-2-oxo-1-phenylethyl 3-amino-6-(1-(1-(3-(4-(2-(3-((1-(2,6-dioxopiperidin-3-yl)-2,5-dioxo-2,5-dihydro-1H-pyrrol-3-yl)amino)phenyl)acetyl)piperazin-1-yl)propyl)piperidin-4-yl)-1H-pyrazol-4-yl)pyrazine-2-carboxylate [TDH-495]

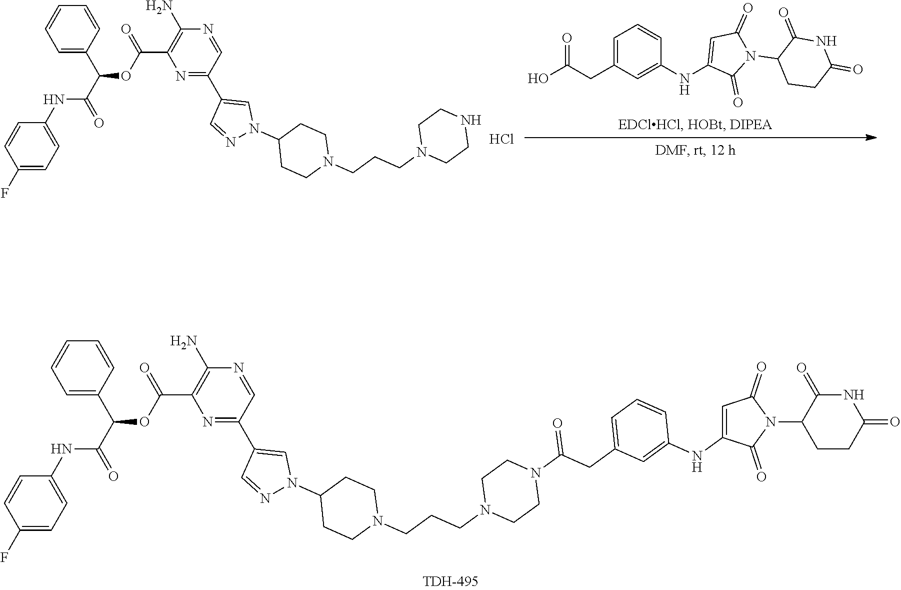 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; O=C(O)Cc1cccc(NC2=CC(=O)N(C3CCC(=O)NC3=O)C2=O)c1 ; Nc1ncc(-c2cnn(C3CCN(CCCN4CCN(C(=O)Cc5cccc(NC6=CC(=O)N(C7CCC(=O)NC7=O)C6=O)c5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (20 mg, 0.030 mmol, 1 eq.), 2-(3-((1-(2,6-dioxopiperidin-3-yl)-2,5-dioxo-2,5-dihydro-1H-pyrrol-3-yl)amino)phenyl)acetic acid (10 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-(2-(3-((1-(2,6-dioxopiperidin-3-yl)-2,5-dioxo-2,5-dihydro-1H-pyrrol-3-yl)amino)phenyl)acetyl)piperazin-1-yl)propyl)piperidin-4-yl)-1H-pyrazol-4-yl)pyrazine-2-carboxylate (19 mg, 67%).

### Synthesis of (R)-2-((4-fluorophenyl)amino)-2-oxo-1-phenylethyl 3-amino-6-(1-(1-(3-(4-(2-((2-(1-methyl-2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate [TDH-525]

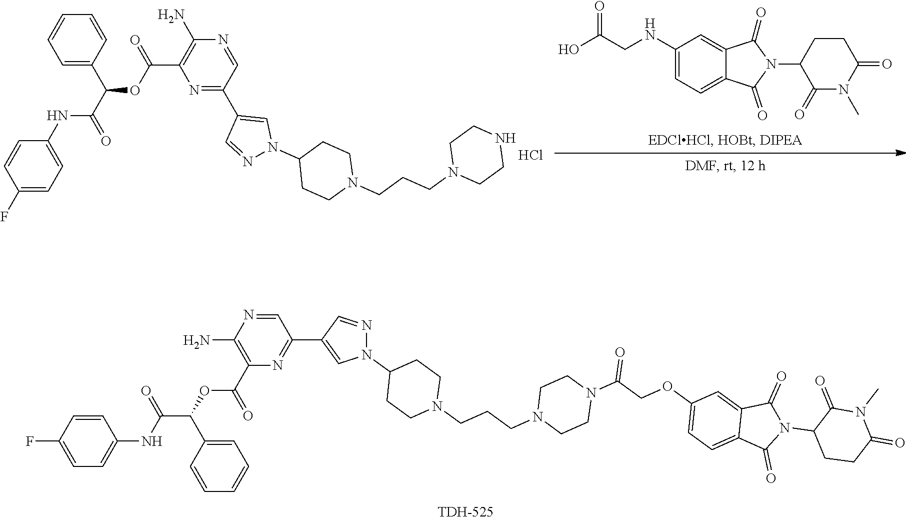 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; CN1C(=O)CCC(N2C(=O)c3ccc(NCC(=O)O)cc3C2=O)C1=O ; CN1C(=O)CCC(N2C(=O)c3ccc(OCC(=O)N4CCN(CCCN5CCC(n6cc(-c7cnc(N)c(C(=O)O[C@@H](C(=O)Nc8ccc(F)cc8)c8ccccc8)n7)cn6)CC5)CC4)cc3C2=O)C1=O]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (67 mg, 0.098 mmol, 1 eq.), 2-((2-(1-methyl-2,6-dioxopiperidin-3-yl)-1,3-dioxoisoindolin-5-yl)oxy)acetic acid (34 mg, 0.098 mmol, 1.0 eq.), EDCI-HCl (21 mg, 0.11 mmol, 1.1 eq.), HOBt (17 mg, 0.11 mmol, 1.1 eq.) and DIPEA (0.09 mL, 0.49 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-(2-((2-(1-methyl-2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate (39 mg, 42%).

### Synthesis of (R)-2-((4-fluorophenyl)amino)-2-oxo-1-phenylethyl 3-amino-6-(1-(1-(3-(4-(1-(1-(2,6-dioxopiperidin-3-yl)-2,5-dioxo-2,5-dihydro-1H-pyrrol-3-yl)piperidine-4-carbonyl)piperazin-1-yl)propyl)piperidin-4-yl)-1H-pyrazol-4-yl)pyrazine-2-carboxylate [TDH-526]

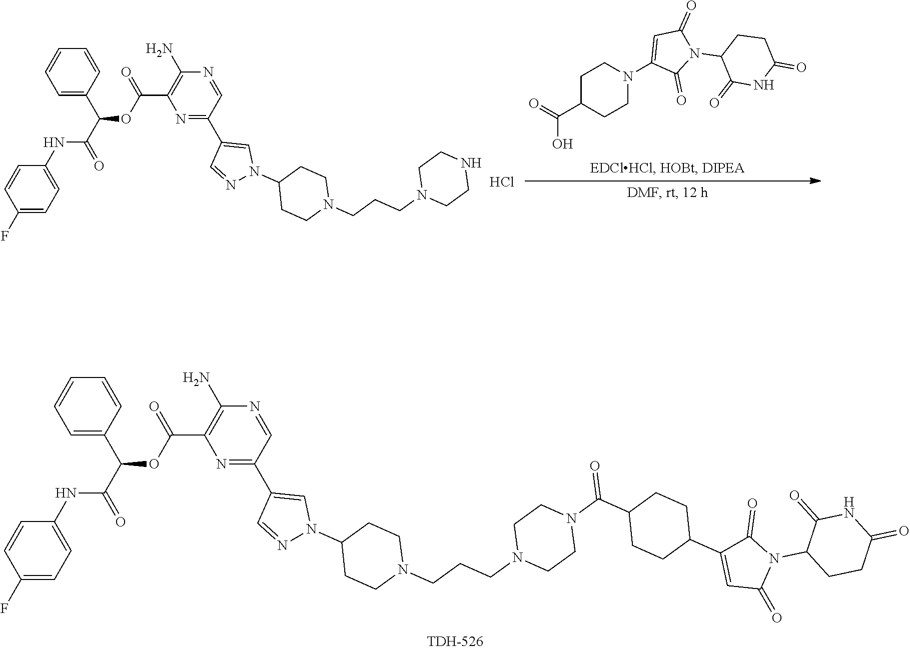 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; O=C1CCC(N2C(=O)C=C(N3CCC(C(=O)O)CC3)C2=O)C(=O)N1 ; Nc1ncc(-c2cnn(C3CCN(CCCN4CCN(C(=O)C5CCC(C6=CC(=O)N(C7CCC(=O)NC7=O)C6=O)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (20 mg, 0.031 mmol, 1 eq.), 1-(1-(2,6-dioxopiperidin-3-yl)-2,5-dioxo-2,5-dihydro-1H-pyrrol-3-yl)piperidine-4-carboxylic acid (10 mg, 0.031 mmol, 1.0 eq.), EDCI-HCl (7 mg, 0.034 mmol, 1.1 eq.), HOBt (5 mg, 0.034 mmol, 1.1 eq.) and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-(1-(1-(2,6-dioxopiperidin-3-yl)-2,5-dioxo-2,5-dihydro-1H-pyrrol-3-yl)piperidine-4-carbonyl)piperazin-1-yl)propyl)piperidin-4-yl)-1H-pyrazol-4-yl)pyrazine-2-carboxylate (10 mg, 34%).

### Synthesis of (R)-2-((4-fluorophenyl)amino)-2-oxo-1-phenylethyl 3-amino-6-(1-(1-(3-(4-(2-(2,6-dioxopiperidin-3-yl)-1-oxoisoindoline-5-carbonyl)piperazin-1-yl)propyl)piperidin-4-yl)-1H-pyrazol-4-yl)pyrazine-2-carboxylate [TDH-530]

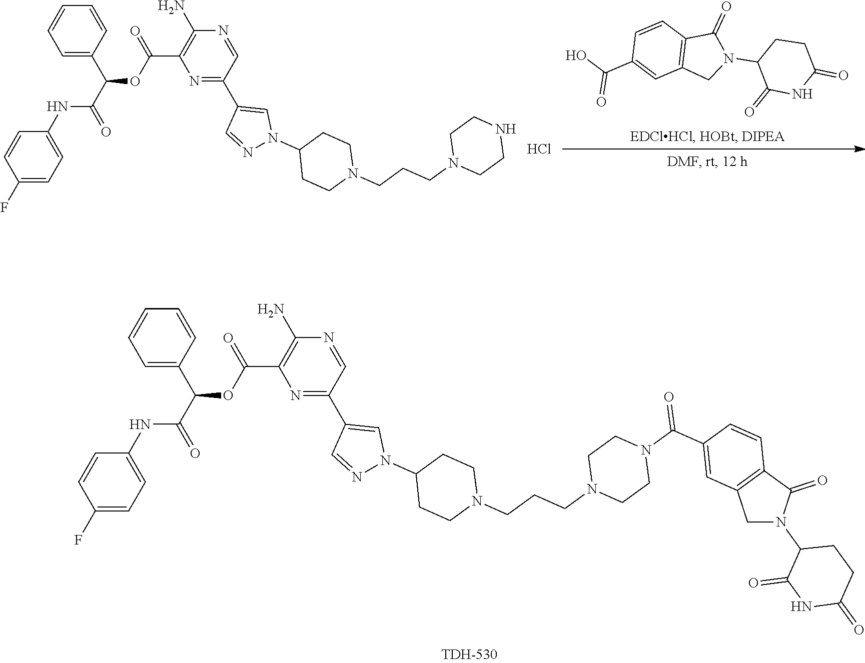 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; O=C1CCC(N2Cc3cc(C(=O)O)ccc3C2=O)C(=O)N1 ; Nc1ncc(-c2cnn(C3CCN(CCCN4CCN(C(=O)c5ccc6c(c5)CN(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (20 mg, 0.031 mmol, 1 eq.), 2-(2,6-dioxopiperidin-3-yl)-1-oxoisoindoline-5-carboxylic acid (9 mg, 0.031 mmol, 1.0 eq.), EDCI-HCl (7 mg, 0.034 mmol, 1.1 eq.), HOBt (5 mg, 0.034 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, ((R)-2-((4-fluorophenyl)amino)-2-oxo-1-phenylethyl 3-amino-6-(1-(1-(3-(4-(2-(2,6-dioxopiperidin-3-yl)-1-oxoisoindoline-5-carbonyl)piperazin-1-yl)propyl)piperidin-4-yl)-1H-pyrazol-4-yl)pyrazine-2-carboxylate (13 mg, 46%).

### Synthesis of (R)-2-((4-fluorophenyl)amino)-2-oxo-1-phenylethyl 3-amino-6-(1-(1-(3-(4-(1-(1-(2,6-dioxopiperidin-3-yl)-2,5-dioxo-2,5-dihydro-1H-pyrrol-3-yl)azetidine-3-carbonyl)piperazin-1-yl)propyl)piperidin-4-yl)-1H-pyrazol-4-yl)pyrazine-2-carboxylate [TDH-532]

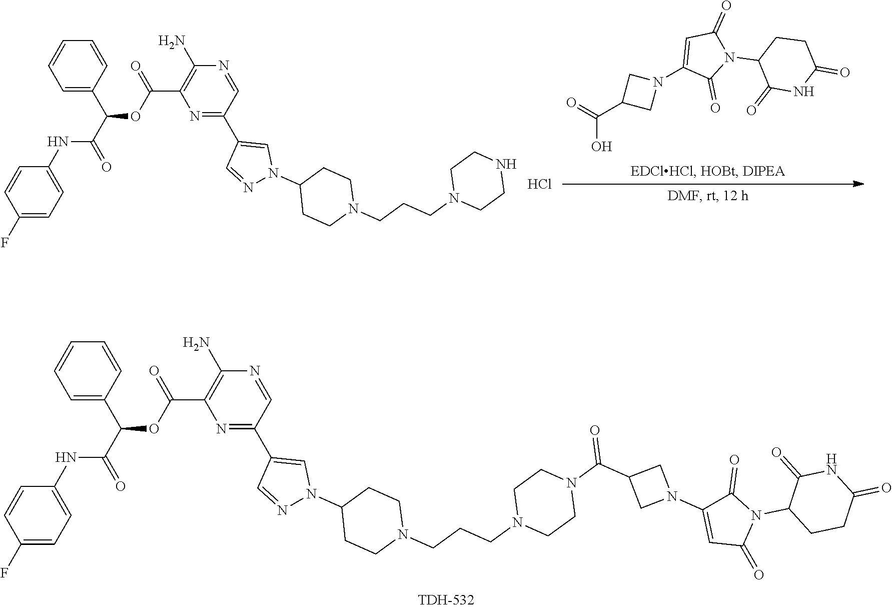 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; O=C1CCC(N2C(=O)C=C(N3CC(C(=O)O)C3)C2=O)C(=O)N1 ; Nc1ncc(-c2cnn(C3CCN(CCCN4CCN(C(=O)C5CN(C6=CC(=O)N(C7CCC(=O)NC7=O)C6=O)C5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (20 mg, 0.031 mmol, 1 eq.), 1-(1-(2,6-dioxopiperidin-3-yl)-2,5-dioxo-2,5-dihydro-1H-pyrrol-3-yl)azetidine-3-carboxylic acid (10 mg, 0.031 mmol, 1.0 eq.), EDCI-HCl (7 mg, 0.034 mmol, 1.1 eq.), HOBt (5 mg, 0.034 mmol, 1.1 eq.) and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-(1-(1-(2,6-dioxopiperidin-3-yl)-2,5-dioxo-2,5-dihydro-1H-pyrrol-3-yl)azetidine-3-carbonyl)piperazin-1-yl)propyl)piperidin-4-yl)-1H-pyrazol-4-yl)pyrazine-2-carboxylate (8 mg, 28%).

### Synthesis of (R)-2-((4-fluorophenyl)amino)-2-oxo-1-phenylethyl 3-amino-6-(1-(1-(3-(4-(4-((2,6-dioxopiperidin-3-yl)carbamoyl)benzoyl)piperazin-1-yl)propyl)piperidin-4-yl)-1H-pyrazol-4-yl)pyrazine-2-carboxylate [TDH-535]

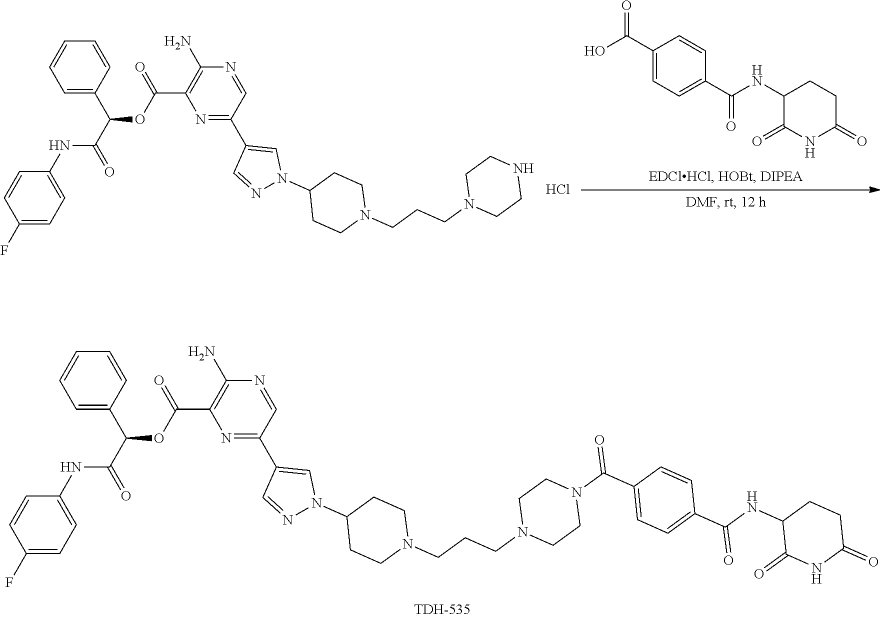 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; O=C1CCC(NC(=O)c2ccc(C(=O)O)cc2)C(=O)N1 ; Nc1ncc(-c2cnn(C3CCN(CCCN4CCN(C(=O)c5ccc(C(=O)NC6CCC(=O)NC6=O)cc5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (20 mg, 0.031 mmol, 1 eq.), 4-((2,6-dioxopiperidin-3-yl)carbamoyl)benzoic acid (9 mg, 0.031 mmol, 1.0 eq.), EDCI-HCl (7 mg, 0.034 mmol, 1.1 eq.), HOBt (5 mg, 0.034 mmol, 1.1 eq.), and DIPEA (0.03 mL, 0.15 mmol, 5 eq.) were dissolved in DMF (1.0 mL), and the mixture was reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-(4-((2,6-dioxopiperidin-3-yl)carbamoyl)benzoyl)piperazin-1-yl)propyl)piperidin-4-yl)-1H-pyrazol-4-yl)pyrazine-2-carboxylate (12 mg, 43%).

### Synthesis of (R)-2-((4-fluorophenyl)amino)-2-oxo-1-phenylethyl 3-amino-6-(1-(1-(3-(4-(1-(1-(2,6-dioxopiperidin-3-yl)-3-methyl-2-oxo-2,3-dihydro-1H-benzo[d]imidazol-5-yl)piperidine-4-carbonyl)piperazin-1-yl)propyl)piperidin-4-yl)-1H-pyrazol-4-yl)pyrazine-2-carboxylate [TDH-540]

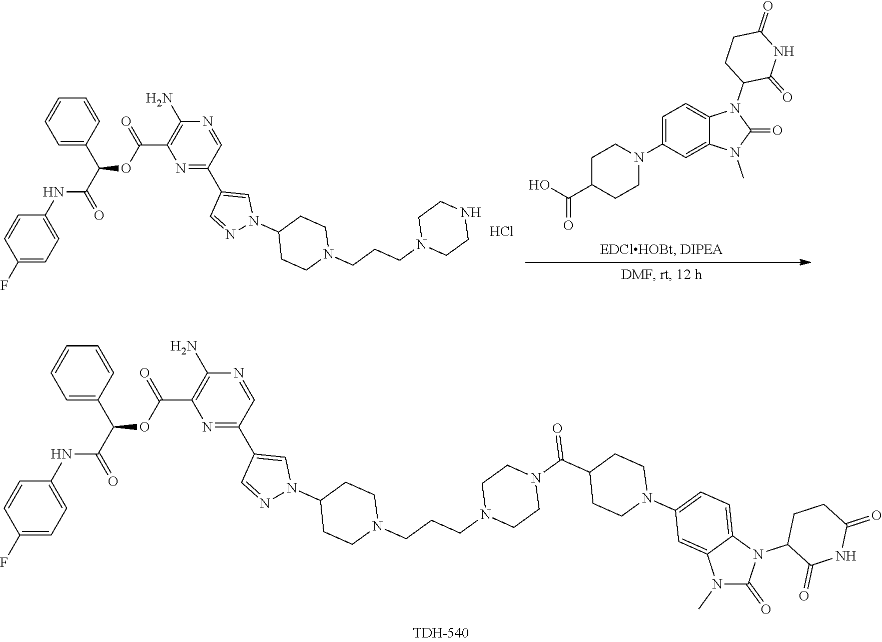 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; Cn1c(=O)n(C2CCC(=O)NC2=O)c2ccc(N3CCC(C(=O)O)CC3)cc21 ; Cn1c(=O)n(C2CCC(=O)NC2=O)c2ccc(N3CCC(C(=O)N4CCN(CCCN5CCC(n6cc(-c7cnc(N)c(C(=O)O[C@@H](C(=O)Nc8ccc(F)cc8)c8ccccc8)n7)cn6)CC5)CC4)CC3)cc21]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (20 mg, 0.031 mmol, 1 eq.), 1-(1-(2,6-dioxopiperidin-3-yl)-3-methyl-2-oxo-2,3-dihydro-1H-benzo[d]imidazol-5-yl)piperidine-4-carboxylic acid (11 mg, 0.031 mmol, 1.0 eq.), EDCI-HCl (7 mg, 0.034 mmol, 1.1 eq.), HOBt (5 mg, 0.034 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-(1-(1-(2,6-dioxopiperidin-3-yl)-3-methyl-2-oxo-2,3-dihydro-1H-benzo[d]imidazol-5-yl)piperidine-4-carbonyl)piperazin-1-yl)propyl)piperidin-4-yl)-1H-pyrazol-4-yl)pyrazine-2-carboxylate (2 mg, 6%).

### Synthesis of (R)-2-((4-fluorophenyl)amino)-2-oxo-1-phenylethyl 3-amino-6-(1-(1-(3-(4-(3-((2,6-dioxopiperidin-3-yl)carbamoyl)benzoyl)piperazin-1-yl)propyl)piperidin-4-yl)-1H-pyrazol-4-yl)pyrazine-2-carboxylate [TDH-542]

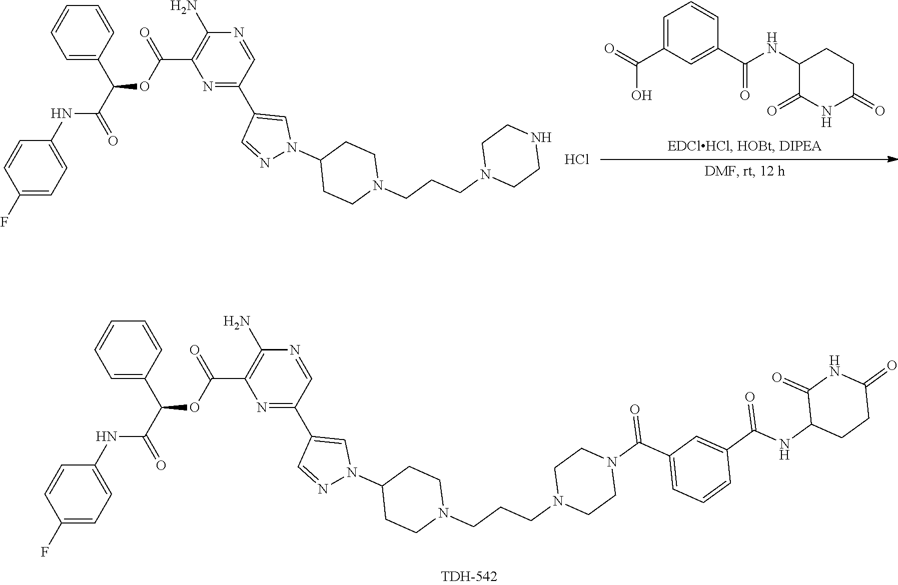 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; O=C1CCC(NC(=O)c2cccc(C(=O)O)c2)C(=O)N1 ; Nc1ncc(-c2cnn(C3CCN(CCCN4CCN(C(=O)c5cccc(C(=O)NC6CCC(=O)NC6=O)c5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (20 mg, 0.031 mmol, 1 eq.), 3-((2,6-dioxopiperidin-3-yl)carbamoyl)benzoic acid (9 mg, 0.031 mmol, 1.0 eq.), EDCI-HCl (7 mg, 0.034 mmol, 1.1 eq.), HOBt (5 mg, 0.034 mmol, 1.1 eq.), and DIPEA (0.03 mL, 0.15 mmol, 5 eq.) were dissolved in DMF (1.0 mL), and the mixture was reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-(3-((2,6-dioxopiperidin-3-yl)carbamoyl)benzoyl)piperazin-1-yl)propyl)piperidin-4-yl)-1H-pyrazol-4-yl)pyrazine-2-carboxylate (16 mg, 57%).

### Synthesis of (R)-2-((4-fluorophenyl)amino)-2-oxo-1-phenylethyl 3-amino-6-(1-(1-(3-(4-(3-(2,4-dioxotetrahydropyrimidin-1(2H)-yl)benzofuran-6-carbonyl)piperazin-1-yl)propyl)piperidin-4-yl)-1H-pyrazol-4-yl)pyrazine-2-carboxylate [TDH-551]

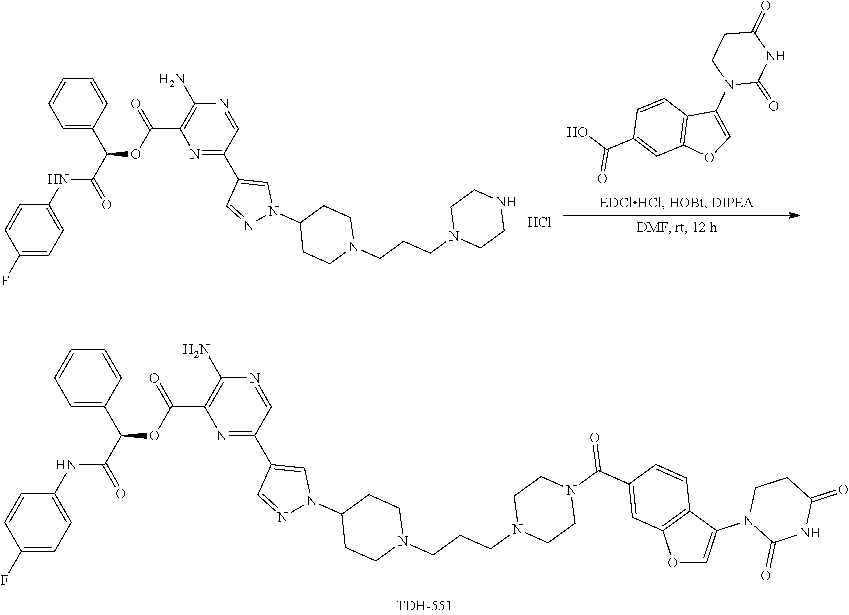 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; O=C1CCN(c2coc3cc(C(=O)O)ccc23)C(=O)N1 ; Nc1ncc(-c2cnn(C3CCN(CCCN4CCN(C(=O)c5ccc6c(N7CCC(=O)NC7=O)coc6c5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (20 mg, 0.031 mmol, 1 eq.), 3-(2,4-dioxotetrahydropyrimidin-1(2H)-yl)benzofuran-6-carboxylic acid (9 mg, 0.031 mmol, 1.0 eq.), EDCI-HCl (7 mg, 0.034 mmol, 1.1 eq.), HOBt (5 mg, 0.034 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-(3-(2,4-dioxotetrahydropyrimidin-1(2H)-yl)benzofuran-6-carbonyl)piperazin-1-yl)propyl)piperidin-4-yl)-1H-pyrazol-4-yl)pyrazine-2-carboxylate (17 mg, 64%).

### Synthesis of (R)-2-((4-fluorophenyl)amino)-2-oxo-1-phenylethyl 3-amino-6-(1-(1-(3-(4-((1-(2-((2-(2,6-dioxopiperidin-3-yl)-1,3-dioxoisoindolin-5-yl)oxy)acetyl)piperidin-4-yl)methyl)piperazin-1-yl)propyl)piperidin-4-yl)-1H-pyrazol-4-yl)pyrazine-2-carboxylate [TDH-440]

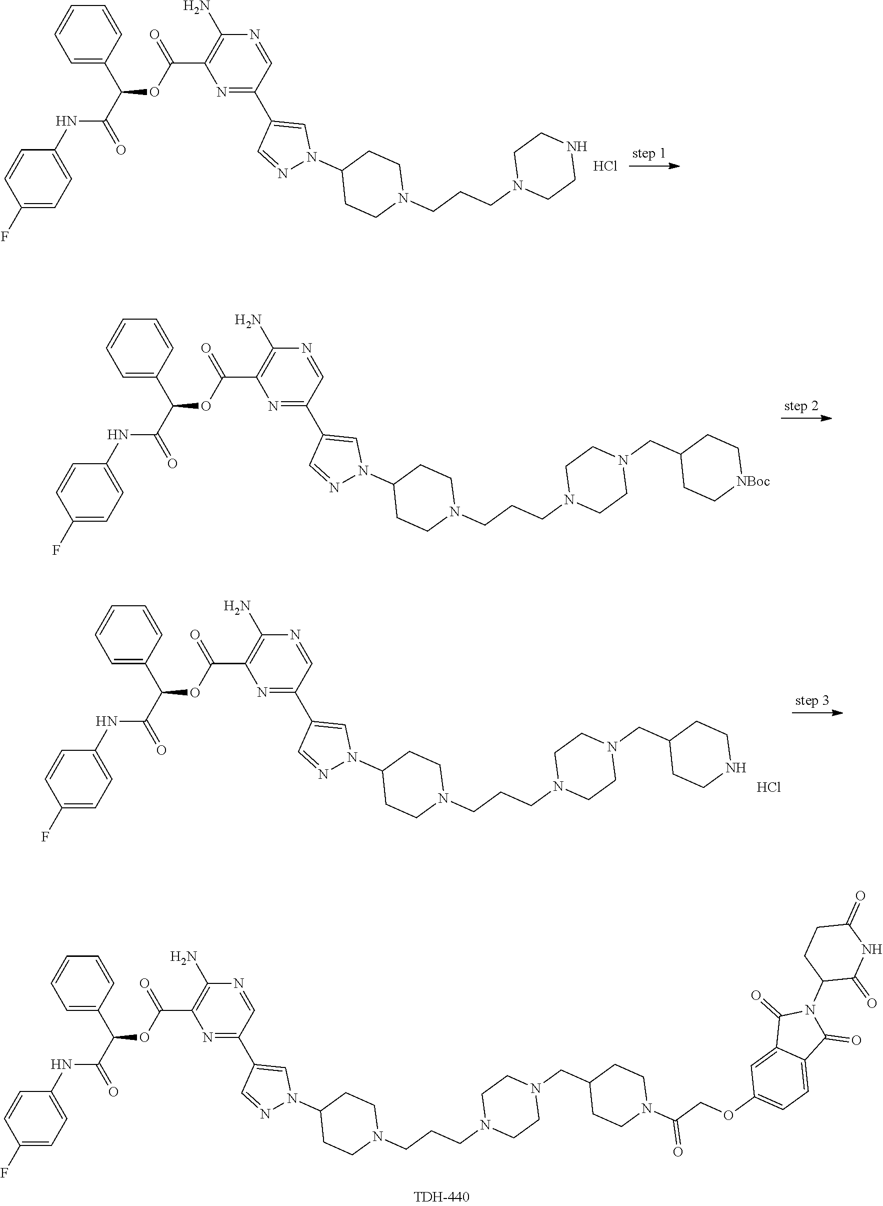 [SMILES: Nc1ncc(-c2cnn(C3CCN(CCCN4CCNCC4)CC3)c2)nc1C(=O)O[C@@H](C(=O)Nc1ccc(F)cc1)c1ccccc1 ; Cl ; CC(C)(C)OC(=O)N1CCC(CN2CCN(CCCN3CCC(n4cc(-c5cnc(N)c(C(=O)O[C@@H](C(=O)Nc6ccc(F)cc6)c6ccccc6)n5)cn4)CC3)CC2)CC1 ; Nc1ncc(-c2cnn(C3CCN(CCCN4CCN(CC5CCNCC5)CC4)CC3)c2)nc1C(=O)O[C@@H](C(=O)Nc1ccc(F)cc1)c1ccccc1 ; Cl ; Nc1ncc(-c2cnn(C3CCN(CCCN4CCN(CC5CCN(C(=O)COc6ccc7c(c6)C(=O)N(C6CCC(=O)NC6=O)C7=O)CC5)CC4)CC3)c2)nc1C(=O)O[C@@H](C(=O)Nc1ccc(F)cc1)c1ccccc1]

### (R)-2-((4-fluorophenyl)amino)-2-oxo-1-phenylethyl 3-amino-6-(1-(1-(3-(4-((1-(tert-butoxycarbonyl)piperidin-4-yl)methyl)piperazin-1-yl)propyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (90 mg, 0.13 mmol, 1 eq.) was dissolved in DCM (1.0 mL), and DIPEA (0.04 mL, 0.20 mmol, 1.5 eq.) was added thereto at 0° C. After 10 minutes, tert-butyl 4-formylpiperidine-1-carboxylate (43 mg, 0.20 mmol, 1.5 eq.) was added to the above solution and reacted at room temperature for 4 hours. After adding NaBH(OAc)₃ (38 mg, 0.18 mmol, 1.34 eq.) at 0° C., the mixture was reacted overnight at room temperature. After completion of the reaction, the mixture was diluted with water and extracted with DCM. The combined organic layer was filtered after removing the residual water through anhydrous Na₂SO₄. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(4-((1-(tert-butoxycarbonyl)piperidin-4-yl)methyl)piperazin-1-yl)propyl)piperidin-4-yl)-1H-pyrazol-4-yl)pyrazine-2-carboxylate (85 mg, 78%).

¹H NMR (400 MHz, CDCl₃) δ 9.00 (s, 1H), 8.53 (s, 1H), 7.99 (s, 1H), 7.94 (s, 1H), 7.68-7.63 (m, 2H), 7.61-7.55 (m, 2H), 7.47-7.40 (m, 3H), 7.09-7.01 (m, 2H), 6.44 (s, 1H), 6.37 (s, 2H), 4.29-4.19 (m, 2H), 4.10 (s, 3H), 3.73-3.63 (m, 1H), 3.18-3.11 (m, 2H), 2.77-2.70 (m, 4H), 2.66-2.62 (m, 4H), 2.54-2.50 (m, 2H), 2.28-2.24 (m, 4H), 2.15-2.09 (m, 2H), 1.93-1.87 (m, 2H), 1.77-1.69 (m, 3H), 1.66-1.62 (m, 1H), 1.51-1.50 (m, 2H), 1.48-1.47 (m, 9H), 1.15-1.06 (m, 2H).

### (R)-2-((4-fluorophenyl)amino)-2-oxo-1-phenylethyl 3-amino-6-(1-(1-(3-(4-(piperidin-4-ylmethyl)piperazin-1-yl)propyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-(3-(4-((1-(tert-butoxycarbonyl)piperidin-4-yl)methyl)piperazin-1-yl)propyl)piperidin-4-yl)-1H-pyrazol-4-yl)pyrazine-2-carboxylate (85 mg, 0.10 mmol) was dissolved in DCM (1.0 mL). Then, 4 N HCl in dioxane (0.5 mL) was slowly added and reacted at room temperature for 1 hour. After completion of the reaction, the precipitate was filtered to obtain the desired (R)-2-((4-fluorophenyl)amino)-2-oxo-1-phenylethyl 3-amino-6-(1-(1-(3-(4-(piperidin-4-ylmethyl)piperazin-1-yl)propyl)piperidin-4-yl)-1H-pyrazol-4-yl)pyrazine-2-carboxylate hydrochloride (70 mg, mixture).

### (R)-2-((4-fluorophenyl)amino)-2-oxo-1-phenylethyl 3-amino-6-(1-(1-(3-(4-((1-(2-((2-(2,6-dioxopiperidin-3-yl)-1,3-dioxoisoindolin-5-yl)oxy)acetyl)piperidin-4-yl)methyl)piperazin-1-yl)propyl)piperidin-4-yl)-1H-pyrazol-4-yl)pyrazine-2-carboxylate [step 3, TDH-440]

(R)-2-((4-fluorophenyl)amino)-2-oxo-1-phenylethyl 3-amino-6-(1-(1-(3-(4-(piperidin-4-ylmethyl)piperazin-1-yl)propyl)piperidin-4-yl)-1H-pyrazol-4-yl)pyrazine-2-carboxylate hydrochloride (15 mg, 0.020 mmol, 1 eq.), 2-((2-(2,6-dioxopiperidin-3-yl)-1,3-dioxoisoindolin-5-yl)oxy)acetic acid (7 mg, 0.020 mmol, 1.0 eq.), EDCI-HCl (5 mg, 0.022 mmol, 1.1 eq.), HOBt (4 mg, 0.022 mmol, 1.1 eq.) and DIPEA (0.02 mL, 0.10 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure and separated and purified by silica gel column chromatography to obtain a yellow solid, (R)-2-((4-fluorophenyl)amino)-2-oxo-1-phenylethyl 3-amino-6-(1-(1-(3-(4-((1-(2-((2-(2,6-dioxopiperidin-3-yl)-1,3-dioxoisoindolin-5-yl)oxy)acetyl)piperidin-4-yl)methyl)piperazin-1-yl)propyl)piperidin-4-yl)-1H-pyrazol-4-yl)pyrazine-2-carboxylate (12 mg, 59%).

### Synthesis of (R)-2-((4-fluorophenyl)amino)-2-oxo-1-phenylethyl 3-amino-6-(1-(1-(3-(4-((1-((2-(2,6-dioxopiperidin-3-yl)-1,3-dioxoisoindolin-5-yl)glycyl)piperidin-4-yl)methyl)piperazin-1-yl)propyl)piperidin-4-yl)-1H-pyrazol-4-yl)pyrazine-2-carboxylate [TDH-441]

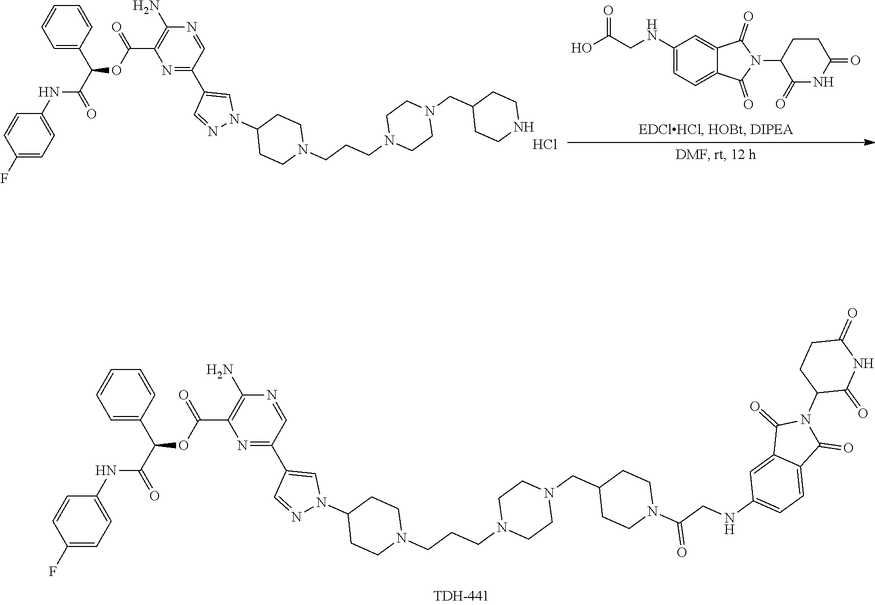 [SMILES: Nc1ncc(-c2cnn(C3CCN(CCCN4CCN(CC5CCNCC5)CC4)CC3)c2)nc1C(=O)O[C@@H](C(=O)Nc1ccc(F)cc1)c1ccccc1 ; Cl ; O=C(O)CNc1ccc2c(c1)C(=O)N(C1CCC(=O)NC1=O)C2=O ; Nc1ncc(-c2cnn(C3CCN(CCCN4CCN(CC5CCN(C(=O)CNc6ccc7c(c6)C(=O)N(C6CCC(=O)NC6=O)C7=O)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(4-(piperidin-4-ylmethyl)piperazin-1-yl)propyl)piperidin-4-yl)-1H-pyrazol-4-yl)pyrazine-2-carboxylate hydrochloride (15 mg, 0.020 mmol, 1 eq.), (2-(2,6-dioxopiperidin-3-yl)-1,3-dioxoisoindolin-5-yl)glycine (7 mg, 0.020 mmol, 1.0 eq.), EDCI-HCl (5 mg, 0.022 mmol, 1.1 eq.), HOBt (4 mg, 0.022 mmol, 1.1 eq.) and DIPEA (0.02 mL, 0.10 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure and separated and purified by silica gel column chromatography to obtain a yellow solid, (R)-2-((4-fluorophenyl)amino)-2-oxo-1-phenylethyl 3-amino-6-(1-(1-(3-(4-((1-((2-(2,6-dioxopiperidin-3-yl)-1,3-dioxoisoindolin-5-yl)glycyl)piperidin-4-yl)methyl)piperazin-1-yl)propyl)piperidin-4-yl)-1H-pyrazol-4-yl)pyrazine-2-carboxylate (13 mg, 62%).

### Synthesis of (R)-2-((4-fluorophenyl)amino)-2-oxo-1-phenylethyl 3-amino-6-(1-(1-(3-(4-((1-(2-(2,6-dioxopiperidin-3-yl)-1,3-dioxoisoindolin-5-yl)piperidin-4-yl)methyl)piperazin-1-yl)propyl)piperidin-4-yl)-1H-pyrazol-4-yl)pyrazine-2-carboxylate [TDH-474]

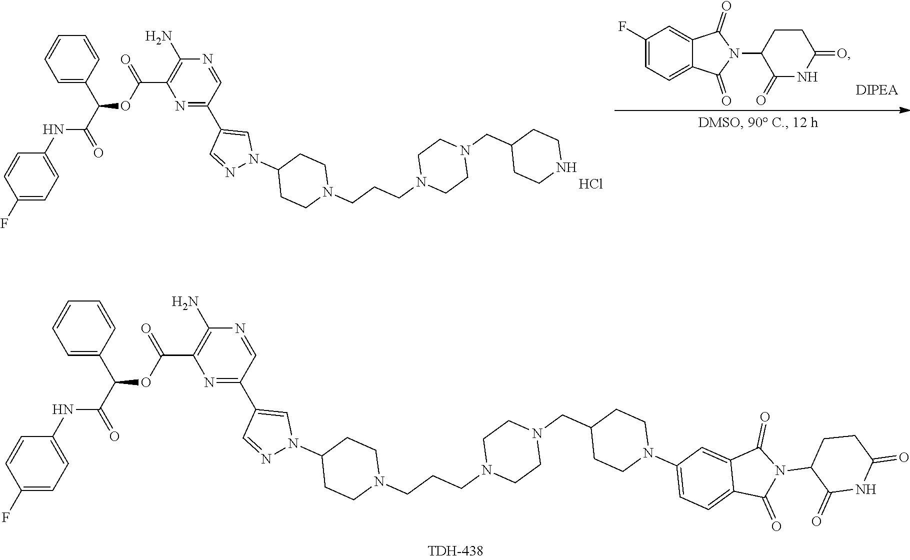 [SMILES: Nc1ncc(-c2cnn(C3CCN(CCCN4CCN(CC5CCNCC5)CC4)CC3)c2)nc1C(=O)O[C@@H](C(=O)Nc1ccc(F)cc1)c1ccccc1 ; Cl ; O=C1CCC(N2C(=O)c3ccc(F)cc3C2=O)C(=O)N1 ; Nc1ncc(-c2cnn(C3CCN(CCCN4CCN(CC5CCN(c6ccc7c(c6)C(=O)N(C6CCC(=O)NC6=O)C7=O)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(4-(piperidin-4-ylmethyl)piperazin-1-yl)propyl)piperidin-4-yl)-1H-pyrazol-4-yl)pyrazine-2-carboxylate hydrochloride (20 mg, 0.026 mmol, 1 eq.), 2-(2,6-dioxopiperidin-3-yl)-5-fluoroisoindoline-1,3-dione (7 mg, 0.026 mmol, 1 eq.) and DIPEA (0.013 mL, 0.077 mmol, 3 eq.) were dissolved in DMSO (1.0 mL) and reacted overnight at 90° C. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, 3-amino-6-(1-(1-(3-(4-(2-(2,6-dioxopiperidin-3-yl)-1,3-dioxoisoindolin-5-yl)piperazin-1-yl)propyl)piperidin-4-yl)-1H-pyrazol-4-yl)-N—((R)-2-((4-fluorophenyl)amino)-2-oxo-1-phenylethyl)pyrazine-2-carboxamide (8 mg, 34%).

### Synthesis of (R)-2-((4-fluorophenyl)amino)-2-oxo-1-phenylethyl 3-amino-6-(1-(1-(3-(4-((1-(3-(2,4-dioxotetrahydropyrimidin-1(2H)-yl)-4-methoxybenzoyl)piperidin-4-yl)methyl)piperazin-1-yl)propyl)piperidin-4-yl)-1H-pyrazol-4-yl)pyrazine-2-carboxylate [TDH-478]

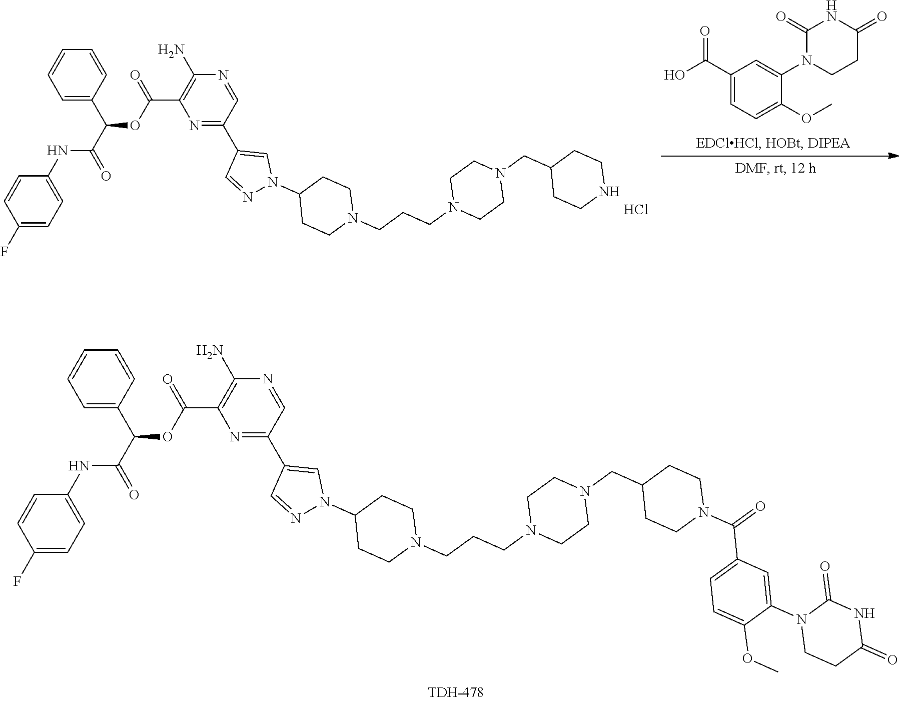 [SMILES: Nc1ncc(-c2cnn(C3CCN(CCCN4CCN(CC5CCNCC5)CC4)CC3)c2)nc1C(=O)O[C@@H](C(=O)Nc1ccc(F)cc1)c1ccccc1 ; Cl ; COc1ccc(C(=O)O)cc1N1CCC(=O)NC1=O ; COc1ccc(C(=O)N2CCC(CN3CCN(CCCN4CCC(n5cc(-c6cnc(N)c(C(=O)O[C@@H](C(=O)Nc7ccc(F)cc7)c7ccccc7)n6)cn5)CC4)CC3)CC2)cc1N1CCC(=O)NC1=O]

(R)-2-((4-fluorophenyl)amino)-2-oxo-1-phenylethyl 3-amino-6-(1-(1-(3-(4-(piperidin-4-ylmethyl)piperazin-1-yl)propyl)piperidin-4-yl)-1H-pyrazol-4-yl)pyrazine-2-carboxylate hydrochloride (15 mg, 0.020 mmol, 1 eq.), 3-(2,4-dioxotetrahydropyrimidin-1(2H)-yl)-4-methoxybenzoic acid (5 mg, 0.020 mmol, 1.0 eq.), EDCI-HCl (5 mg, 0.022 mmol, 1.1 eq.), HOBt (4 mg, 0.022 mmol, 1.1 eq.), and DIPEA (0.02 mL, 0.10 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure and separated and purified by silica gel column chromatography to obtain a yellow solid, (R)-2-((4-fluorophenyl)amino)-2-oxo-1-phenylethyl 3-amino-6-(1-(1-(3-(4-((1-(3-(2,4-dioxotetrahydropyrimidin-1(2H)-yl)-4-methoxybenzoyl)piperidin-4-yl)methyl)piperazin-1-yl)propyl)piperidin-4-yl)-1H-pyrazol-4-yl)pyrazine-2-carboxylate (6 mg, 30%).

### Synthesis of (R)-2-((3-chlorophenyl)amino)-2-oxo-1-phenylethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate [TDH-485]

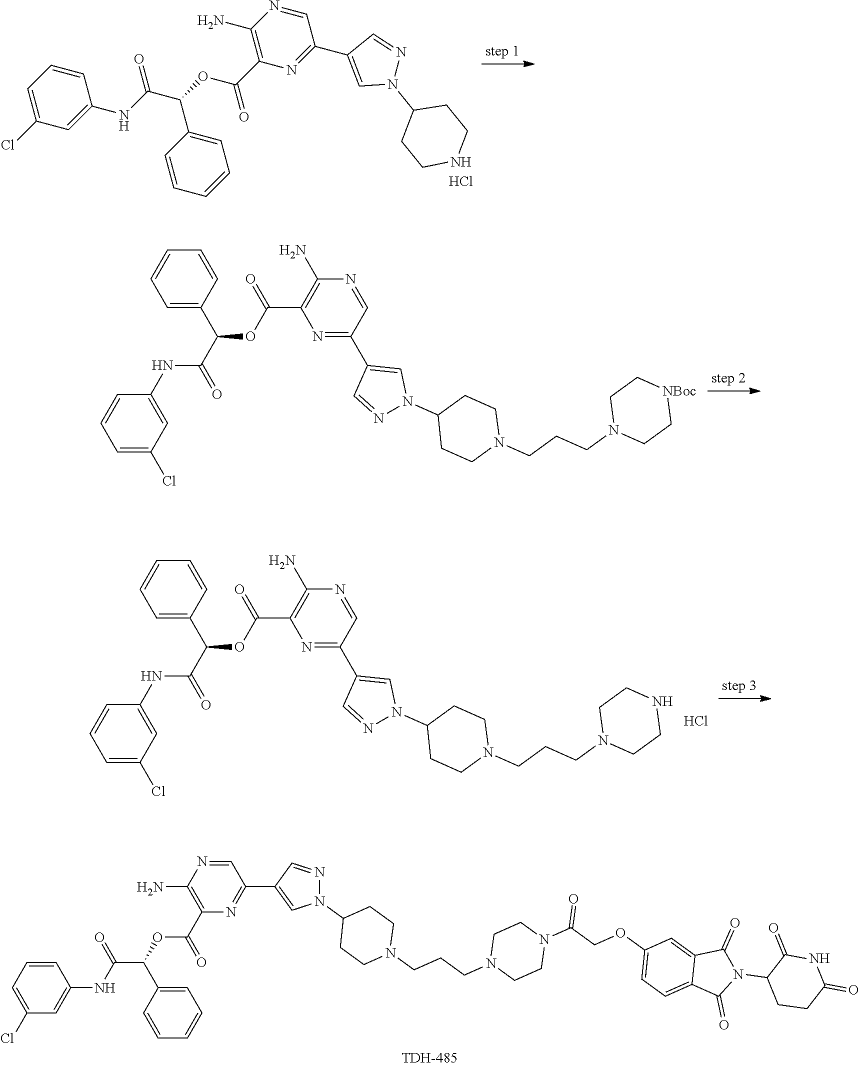 [SMILES: Nc1ncc(-c2cnn(C3CCNCC3)c2)nc1C(=O)O[C@@H](C(=O)Nc1cccc(Cl)c1)c1ccccc1 ; Cl ; CC(C)(C)OC(=O)N1CCN(CCCN2CCC(n3cc(-c4cnc(N)c(C(=O)O[C@@H](C(=O)Nc5cccc(Cl)c5)c5ccccc5)n4)cn3)CC2)CC1 ; Nc1ncc(-c2cnn(C3CCN(CCCN4CCNCC4)CC3)c2)nc1C(=O)O[C@@H](C(=O)Nc1cccc(Cl)c1)c1ccccc1 ; Cl ; Nc1ncc(-c2cnn(C3CCN(CCCN4CCN(C(=O)COc5ccc6c(c5)C(=O)N(C5CCC(=O)NC5=O)C6=O)CC4)CC3)c2)nc1C(=O)O[C@@H](C(=O)Nc1cccc(Cl)c1)c1ccccc1]

### Synthesis of (R)-2-((3-chlorophenyl)amino)-2-oxo-1-phenylethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (step 1)

(R)-2-((3-chlorophenyl)amino)-2-oxo-1-phenylethyl 3-amino-6-(1-(piperidin-4-yl)-1H-pyrazol-4-yl)pyrazine-2-carboxylate hydrochloride (15 mg, 0.026 mmol, 1.0 eq.), tert-butyl 4-(3-iodopropyl)piperazine-1-carboxylate (14 mg, 0.040 mmol, 1.5 eq.), and K₂CO₃ (7 mg, 0.053 mmol, 2.0 eq.) were dissolved in DMF (1 mL) and reacted overnight at 50° C. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((3-chlorophenyl)amino)-2-oxo-1-phenylethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (11 mg, 69%).

¹H NMR (300 MHz, CDCl₃) δ 9.04 (s, 1H), 8.54 (s, 1H), 7.99 (s, 1H), 7.95 (s, 1H), 7.71-7.67 (m, 1H), 7.67-7.62 (m, 2H), 7.49-7.39 (m, 4H), 7.26-7.22 (m, 1H), 7.14-7.08 (m, 1H), 6.45 (s, 1H), 6.36 (s, 2H), 4.32-4.13 (m, 1H), 3.50-3.42 (m, 4H), 3.21-3.05 (m, 2H), 2.52-2.40 (m, 8H), 2.25-2.03 (m, 6H), 1.82-1.71 (m, 2H), 1.48 (s, 9H).

### Synthesis of (R)-2-((3-chlorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (step 2)

(R)-2-((3-chlorophenyl)amino)-2-oxo-1-phenylethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (11 mg, 0.014 mmol) was dissolved in DCM (1.0 mL). Then, 4 M HCl in dioxane (0.5 mL) was added, and reacted at room temperature for 2 hours. The reaction solvent was concentrated to obtain a yellow solid, (R)-2-((3-chlorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (12 mg, mixture).

### Synthesis of (R)-2-((3-chlorophenyl)amino)-2-oxo-1-phenylethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate [step 3, TDH-485]

(R)-2-((3-chlorophenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (12 mg, 0.017 mmol, 1 eq.), 2-((2-(2,6-dioxopiperidin-3-yl)-1,3-dioxoisoindolin-5-yl)oxy)acetic acid (6 mg, 0.017 mmol, 1.0 eq.), EDCI-HCl (4 mg, 0.019 mmol, 1.1 eq.), HOBt (3 mg, 0.019 mmol, 1.1 eq.), and DIPEA (0.02 mL, 0.086 mmol, 5 eq.) were dissolved in DMF (1.0 mL), and the mixture was reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((3-chlorophenyl)amino)-2-oxo-1-phenylethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate (11 mg, 67%).

### Synthesis of (R)-2-((3-methoxyphenyl)amino)-2-oxo-1-phenylethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate [TDH-486]

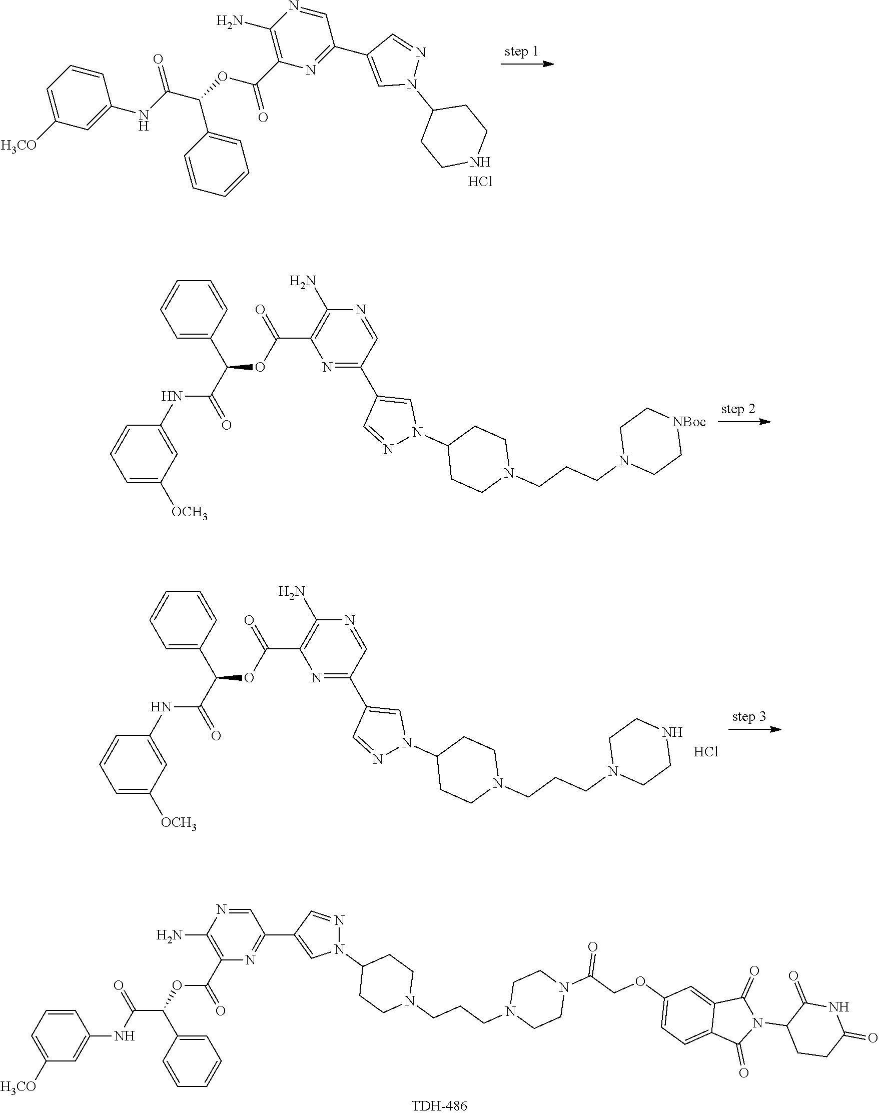 [SMILES: COc1cccc(NC(=O)[C@H](OC(=O)c2nc(-c3cnn(C4CCNCC4)c3)cnc2N)c2ccccc2)c1 ; Cl ; COc1cccc(NC(=O)[C@H](OC(=O)c2nc(-c3cnn(C4CCN(CCCN5CCN(C(=O)OC(C)(C)C)CC5)CC4)c3)cnc2N)c2ccccc2)c1 ; COc1cccc(NC(=O)[C@H](OC(=O)c2nc(-c3cnn(C4CCN(CCCN5CCNCC5)CC4)c3)cnc2N)c2ccccc2)c1 ; Cl ; COc1cccc(NC(=O)[C@H](OC(=O)c2nc(-c3cnn(C4CCN(CCCN5CCN(C(=O)COc6ccc7c(c6)C(=O)N(C6CCC(=O)NC6=O)C7=O)CC5)CC4)c3)cnc2N)c2ccccc2)c1]

### Synthesis of (R)-2-((3-methoxyphenyl)amino)-2-oxo-1-phenylethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (step 1)

(R)-2-((3-((λ¹-oxidaneyl)-λ⁵-methyl)phenyl)amino)-2-oxo-1-phenylethyl 3-amino-6-(1-(piperidin-4-yl)-1H-pyrazol-4-yl)pyrazine-2-carboxylate hydrochloride (15 mg, 0.027 mmol, 1.0 eq.), tert-butyl 4-(3-iodopropyl)piperazine-1-carboxylate (14 mg, 0.040 mmol, 1.5 eq.), and K₂CO₃ (7 mg, 0.053 mmol, 2.0 eq.) were dissolved in DMF (1 mL) and reacted at 50° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((3-methoxyphenyl)amino)-2-oxo-1-phenylethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (6 mg, 29%).

¹H NMR (300 MHz, CDCl₃) δ 8.95 (s, 1H), 8.53 (s, 1H), 8.01 (s, 1H), 7.97 (s, 1H), 7.69-7.62 (m, 2H), 7.48-7.38 (m, 4H), 7.28-7.18 (m, 1H), 7.06-7.00 (m, 1H), 6.74-6.67 (m, 1H), 6.43 (s, 1H), 6.34 (s, 2H), 4.27-4.19 (m, 1H), 3.77 (s, 3H), 3.49-3.44 (m, 4H), 3.15-3.06 (m, 2H), 2.46-2.41 (m, 6H), 2.31-2.14 (m, 8H), 1.79-1.73 (m, 2H), 1.48 (s, 9H).

### Synthesis of (R)-2-((3-methoxyphenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (step 2)

(R)-2-((3-methoxyphenyl)amino)-2-oxo-1-phenylethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (6 mg, 0.008 mmol) was dissolved in DCM (1.0 mL) and 4 M HCl in dioxane (0.5 mL) was added. Then, it was reacted at room temperature for 2 hours. The reaction solvent was concentrated to obtain a yellow solid, (R)-2-((3-methoxyphenyl)amino)-2-oxo-1-phenylethyl 3-amino-6-(1-(1-(3-(piperazin-1-yl))propyl)piperidin-4-yl)-1H-pyrazol-4-yl)pyrazine-2-carboxylate hydrochloride (7 mg, mixture).

### Synthesis of (R)-2-((3-methoxyphenyl)amino)-2-oxo-1-phenylethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate [step 3, TDH-486]

(R)-2-((3-methoxyphenyl)amino)-2-oxo-1-phenylethyl 3-amino-6-(1-(1-(3-(piperazin-1-yl)propyl)piperidin-4-yl)-1H-pyrazol-4-yl)pyrazine-2-carboxylate hydrochloride (7 mg, 0.011 mmol, 1 eq.), 2-((2-(2,6-dioxopiperidin-3-yl)-1,3-dioxoisoindolin-5-yl)oxy)acetic acid (4 mg, 0.011 mmol, 1.0 eq.), EDCI-HCl (3 mg, 0.012 mmol, 1.1 eq.), HOBt (2 mg, 0.012 mmol, 1.1 eq.), and DIPEA (0.01 mL, 0.056 mmol, 5 eq.) were dissolved in DMF (1.0 mL), and the mixture was reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((3-methoxyphenyl)amino)-2-oxo-1-phenylethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate (6 mg, 56%).

### Synthesis of (R)-2-oxo-1-phenyl-2-(m-tolylamino)ethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate [TDH-487]

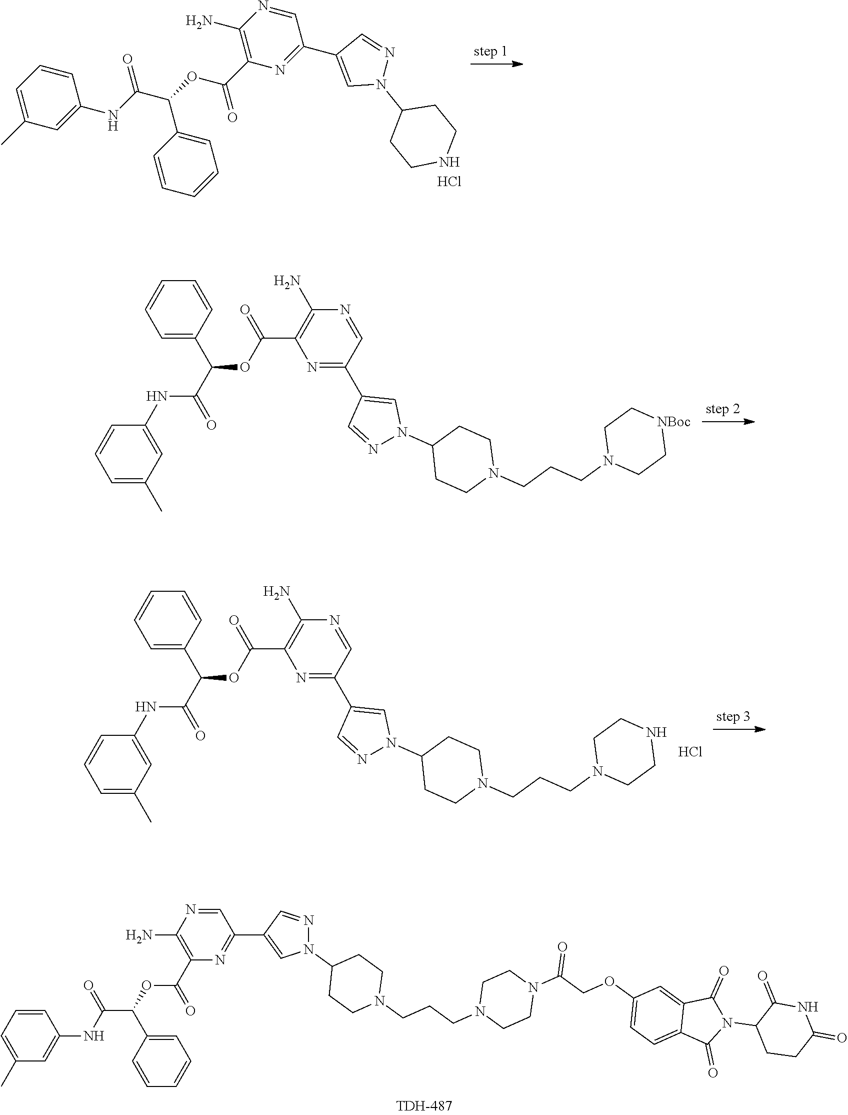 [SMILES: Cc1cccc(NC(=O)[C@H](OC(=O)c2nc(-c3cnn(C4CCNCC4)c3)cnc2N)c2ccccc2)c1 ; Cl ; Cc1cccc(NC(=O)[C@H](OC(=O)c2nc(-c3cnn(C4CCN(CCCN5CCN(C(=O)OC(C)(C)C)CC5)CC4)c3)cnc2N)c2ccccc2)c1 ; Cc1cccc(NC(=O)[C@H](OC(=O)c2nc(-c3cnn(C4CCN(CCCN5CCNCC5)CC4)c3)cnc2N)c2ccccc2)c1 ; Cl ; Cc1cccc(NC(=O)[C@H](OC(=O)c2nc(-c3cnn(C4CCN(CCCN5CCN(C(=O)COc6ccc7c(c6)C(=O)N(C6CCC(=O)NC6=O)C7=O)CC5)CC4)c3)cnc2N)c2ccccc2)c1]

### Synthesis of (R)-2-oxo-1-phenyl-2-(m-tolylamino)ethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (step 1)

(R)-2-oxo-1-phenyl-2-(m-tolylamino)ethyl 3-amino-6-(1-(piperidin-4-yl)-1H-pyrazol-4-yl)pyrazine-2-carboxylate hydrochloride (15 mg, 0.027 mmol, 1.0 eq.), tert-butyl 4-(3-iodopropyl)piperazine-1-carboxylate (14 mg, 0.040 mmol, 1.5 eq.), and K₂CO₃ (7 mg, 0.053 mmol, 2.0 eq.) were dissolved in DMF (1 mL) and reacted overnight at 50° C. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-oxo-1-phenyl-2-(m-tolylamino)ethyl 3-amino-6-(1-(1-(3)-(4-(tert-butoxycarbonyl)piperazin-1-yl)propyl)piperidin-4-yl)-1H-pyrazol-4-yl)pyrazine-2-carboxylate (21 mg, mixture).

### Synthesis of (R)-2-oxo-1-phenyl-2-(m-tolylamino)ethyl 3-amino-6-(1-(1-(3-(piperazin-1-yl)propyl)piperidin-4-yl)-1H-pyrazol-4-yl)pyrazine-2-carboxylate hydrochloride (step 2)

(R)-2-oxo-1-phenyl-2-(m-tolylamino)ethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (21 mg, 0.028 mmol) was dissolved in DCM (2.0 mL), 4 M HCl in dioxane (1.0 mL) was added, and it was reacted at room temperature for 2 hours. The reaction solvent was concentrated to obtain a yellow solid, (R)-2-oxo-1-phenyl-2-(m-tolylamino)ethyl 3-amino-6-(1-(1-(3-(piperazin-1-yl)propyl)piperidin-4-yl)-1H-pyrazol-4-yl)pyrazine-2-carboxylate hydrochloride (21 mg, mixture).

### Synthesis of (R)-2-oxo-1-phenyl-2-(m-tolylamino)ethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate [step 3, TDH-487]

(R)-2-oxo-1-phenyl-2-(m-tolylamino)ethyl 3-amino-6-(1-(1-(3-(piperazin-1-yl)propyl)piperidin-4-yl)-1H-pyrazol-4-yl)pyrazine-2-carboxylate hydrochloride (21 mg, 0.031 mmol, 1 eq.), 2-((2-(2,6-dioxopiperidin-3-yl)-1,3-dioxoisoindolin-5-yl)oxy)acetic acid (10 mg, 0.031 mmol, 1.0 eq.), EDCI-HCl (7 mg, 0.034 mmol, 1.1 eq.), HOBt (5 mg, 0.034 mmol, 1.1 eq.), and DIPEA (0.03 mL, 0.16 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-oxo-1-phenyl-2-(m-tolylamino)ethyl 3-amino-6-(1-(1-(3)-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate (11 mg, 37%).

### Synthesis of (R)-1-(2-chlorophenyl)-2-((4-fluorophenyl)amino)-2-oxoethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate [TDH-582]

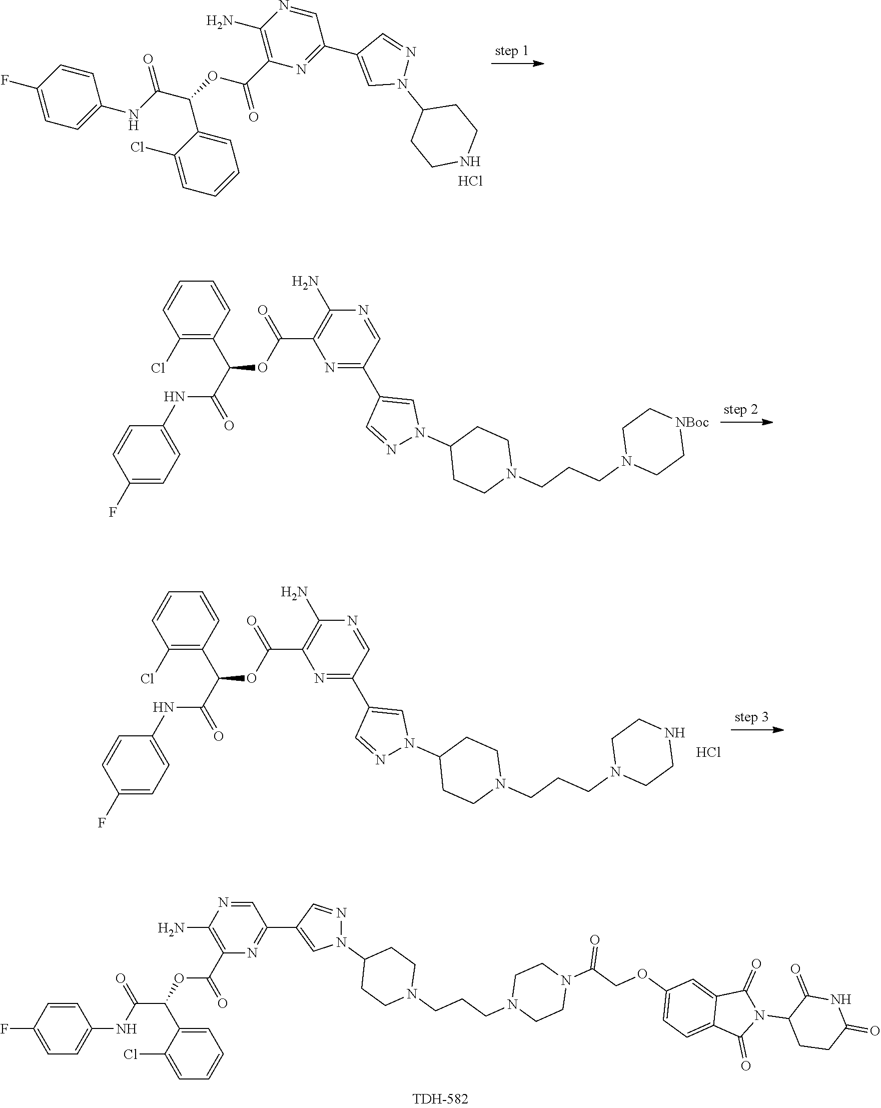 [SMILES: Nc1ncc(-c2cnn(C3CCNCC3)c2)nc1C(=O)O[C@@H](C(=O)Nc1ccc(F)cc1)c1ccccc1Cl ; Cl ; CC(C)(C)OC(=O)N1CCN(CCCN2CCC(n3cc(-c4cnc(N)c(C(=O)O[C@@H](C(=O)Nc5ccc(F)cc5)c5ccccc5Cl)n4)cn3)CC2)CC1 ; Nc1ncc(-c2cnn(C3CCN(CCCN4CCNCC4)CC3)c2)nc1C(=O)O[C@@H](C(=O)Nc1ccc(F)cc1)c1ccccc1Cl ; Cl ; Nc1ncc(-c2cnn(C3CCN(CCCN4CCN(C(=O)COc5ccc6c(c5)C(=O)N(C5CCC(=O)NC5=O)C6=O)CC4)CC3)c2)nc1C(=O)O[C@@H](C(=O)Nc1ccc(F)cc1)c1ccccc1Cl]

### Synthesis of (R)-1-(2-chlorophenyl)-2-((4-fluorophenyl)amino)-2-oxoethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (step 1)

(R)-1-(2-chlorophenyl)-2-((4-fluorophenyl)amino)-2-oxoethyl 3-amino-6-(1-(piperidin-4-yl)-1H-pyrazol-4-yl)pyrazine-2-carboxylate hydrochloride (13 mg, 0.02 mmol, 1.0 eq.), tert-butyl 4-(3-iodopropyl)piperazine-1-carboxylate (12 mg, 0.03 mmol, 1.5 eq.), and K₂CO₃ (6 mg, 0.04 mmol, 2.0 eq.) were dissolved in DMF (1 mL) and reacted at 50° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-1-(2-chlorophenyl)-2-((4-fluorophenyl)amino)-2-oxoethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (19 mg, mixture).

### Synthesis of (R)-1-(2-chlorophenyl)-2-((4-fluorophenyl)amino)-2-oxoethyl 3-amino-6-(1-(1-(3-(piperazin-1-yl)propyl)piperidin-4-yl)-1H-pyrazol-4-yl)pyrazine-2-carboxylate hydrochloride (step 2)

(R)-1-(2-chlorophenyl)-2-((4-fluorophenyl)amino)-2-oxoethyl 3-amino-6-(1-(1-(3-(4-(tert-butoxycarbonyl)piperazin-1-yl)propyl)piperidin-4-yl)-1H-pyrazol-4-yl)pyrazine-2-carboxylate (19 mg, 0.026 mmol) was dissolved in DCM (1.0 mL). Then, 4 M HCl in dioxane (0.5 mL) was added and reacted at room temperature for 2 hours. The reaction solvent was concentrated to obtain a yellow solid, ((R)-1-(2-chlorophenyl)-2-((4-fluorophenyl)amino)-2-oxoethyl 3-amino-6-(1-(1-(3-(piperazin-1-yl)propyl)piperidin-4-yl)-1H-pyrazol-4-yl)pyrazine-2-carboxylate hydrochloride (16 mg, mixture).

### Synthesis of (R)-1-(2-chlorophenyl)-2-((4-fluorophenyl)amino)-2-oxoethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate [step 3, TDH-582]

(R)-1-(2-chlorophenyl)-2-((4-fluorophenyl)amino)-2-oxoethyl 3-amino-6-(1-(1-(3-(piperazin-1-yl)propyl)piperidin-4-yl)-1H-pyrazol-4-yl)pyrazine-2-carboxylate hydrochloride (16 mg, 0.022 mmol, 1 eq.), 2-((2-(2,6-dioxopiperidin-3-yl)-1,3-dioxoisoindolin-5-yl)oxy)acetic acid (7 mg, 0.022 mmol, 1.0 eq.), EDCI-HCl (5 mg, 0.025 mmol, 1.1 eq.), HOBt (4 mg, 0.025 mmol, 1.1 eq.) and DIPEA (0.02 mL, 0.11 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-1-(2-chlorophenyl)-2-((4-fluorophenyl)amino)-2-oxoethyl 3-amino-6-(1-(1-(3-(4-(2-((2-(2,6-dioxopiperidin-3-yl)-1,3-dioxoisoindolin-5-yl)oxy)acetyl)piperazin-1-yl)propyl)piperidin-4-yl)-1H-pyrazol-4-yl)pyrazine-2-carboxylate (16 mg, 76%).

### Synthesis of (R)-2-((4-fluorophenyl)amino)-2-oxo-1-phenylethyl 3-amino-6-(1-(1-(7-((1-(2-((2-(2,6-dioxopiperidin-3-yl)-1,3-dioxoisoindolin-5-yl)oxy)acetyl)piperidin-4-VI)methyl)-7-azaspiro[3.5]nonan-2-yl)piperidin-4-yl)-1H-pyrazol-4-yl)pyrazine-2-carboxylate [TDH-442]

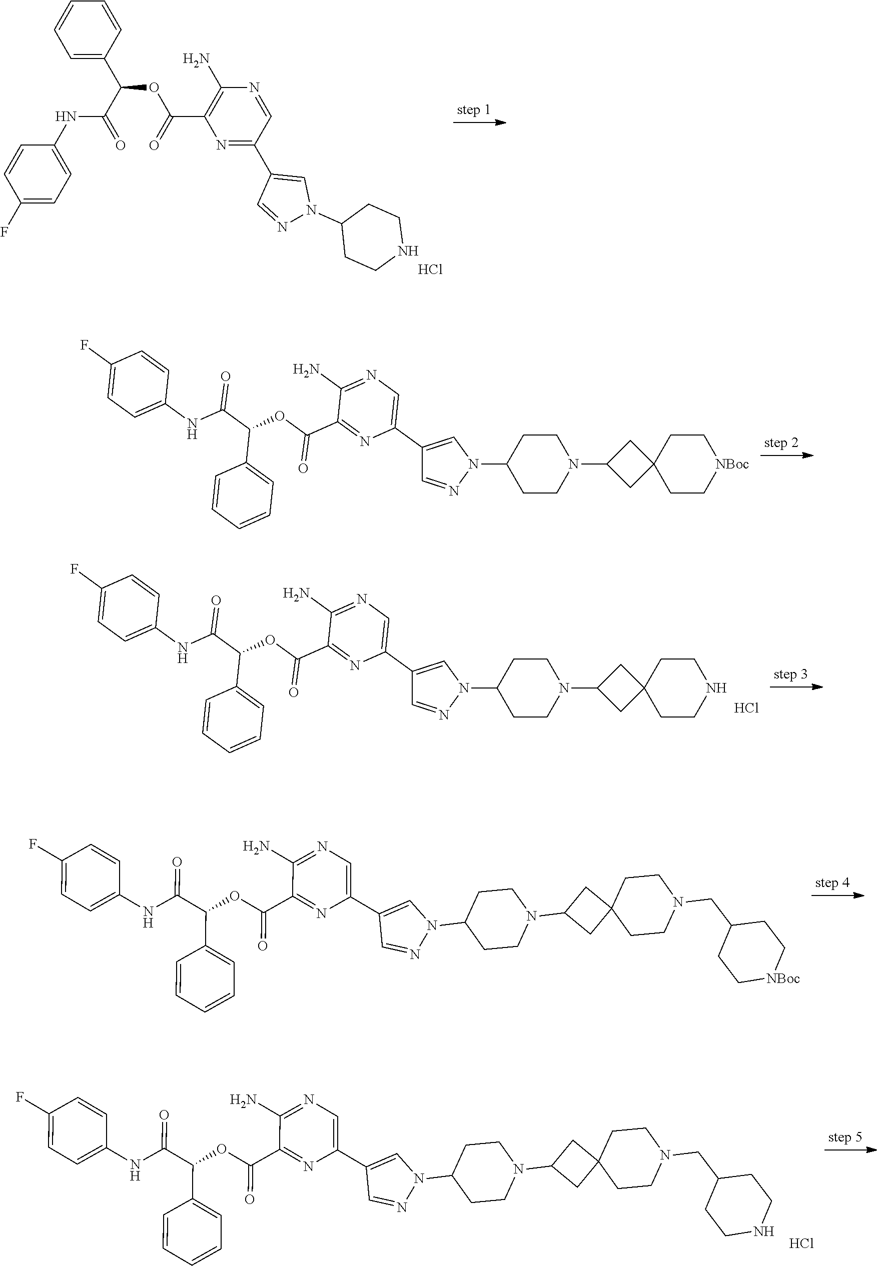

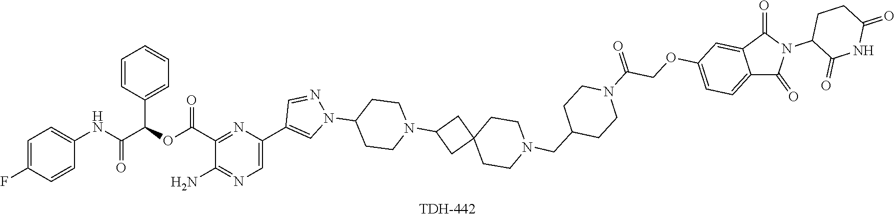 [SMILES: Nc1ncc(-c2cnn(C3CCN(C4CC5(CCN(CC6CCN(C(=O)COc7ccc8c(c7)C(=O)N(C7CCC(=O)NC7=O)C8=O)CC6)CC5)C4)CC3)c2)nc1C(=O)O[C@@H](C(=O)Nc1ccc(F)cc1)c1ccccc1]

### Tert-butyl (R)-2-(4-(4-(5-amino-6-((2-((4-fluorophenyl)amino)-2-oxo-1-phenylethoxy)carbonyl)pyrazin-2-yl)-1H-pyrazol-1-yl)piperidin-1-yl)-7-azaspiro[3.5]nonane-7-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylate hydrochloride (200 mg, 0.36 mmol, 1 eq.) was dissolved in DCM (1.5 mL), and DIPEA (0.1 mL, 0.54 mmol, 1.5 eq.) was added at 0° C. After 10 minutes, tert-butyl 2-oxo-7-azaspiro[3.5]nonane-7-carboxylate (130 mg, 0.54 mmol, 1.5 eq.) was added to the above solution and reacted at room temperature for 4 hours. After adding NaBH(OAc)₃ (103 mg, 0.48 mmol, 1.34 eq.) at 0° C., the mixture was reacted overnight at room temperature. After completion of the reaction, the mixture was diluted with water and extracted with DCM. The combined organic layer was filtered after removing the residual water through anhydrous Na₂SO₄. The organic layer was concentrated under reduced pressure. The concentrate was separated and purified by silica gel column chromatography to obtain the desired yellow solid, tert-butyl (R)-2-(4-(4-(5-amino-6-((2-((4-fluorophenyl)amino)-2-oxo-1-phenylethoxy)carbonyl)pyrazin-2-yl)-1H-pyrazol-1-yl)piperidin-1-yl)-7-azaspiro[3.5]nonane-7-carboxylate (176 mg, 66%).

¹H NMR (300 MHz, CDCl₃) δ 9.03 (s, 1H), 8.52 (s, 1H), 7.98 (s, 2H), 7.71-7.62 (m, 2H), 7.62-7.54 (m, 2H), 7.50-7.37 (m, 3H), 7.10-6.99 (m, 2H), 6.48-6.35 (m, 3H), 4.43-4.27 (m, 2H), 3.44-3.36 (m, 3H), 3.36-3.26 (m, 3H), 3.26-3.16 (m, 3H), 3.16-3.05 (m, 8H), 2.41-2.17 (m, 6H), 2.17-2.09 (m, 6H), 1.47 (s, 9H).

### (R)-2-((4-fluorophenyl)amino)-2-oxo-1-phenylethyl 6-(1-(1-(7-azaspiro[3.5]nonan-2-yl)piperidin-4-yl)-1H-pyrazol-4-yl)-3-aminopyrazine-2-carboxylate hydrochloride (step 2)

Tert-butyl (R)-2-(4-(4-(5-amino-6-((2-((4-fluorophenyl)amino)-2-oxo-1-phenylethoxy)carbonyl)pyrazin-2-yl)-1H-pyrazol-1-yl)piperidin-1-yl)-7-azaspiro[3.5]nonane-7-carboxylate (176 mg, 0.24 mmol) was dissolved in DCM (2.0 mL). Then, 4 N HCl in dioxane (1.0 mL) was added slowly and reacted at room temperature for 1 hour. After completion of the reaction, the precipitate was filtered to obtain the desired (R)-2-((4-fluorophenyl)amino)-2-oxo-1-phenylethyl 6-(1-(1-(7-azaspiro[3.5]nonan-2-yl)piperidin-4-yl)-1H-pyrazol-4-yl)-3-aminopyrazine-2-carboxylate hydrochloride (104 mg, 64%).

¹H NMR (300 MHz, MeOD) δ 8.62 (s, 1H), 8.32-8.26 (m, 1H), 8.08 (s, 1H), 7.78-7.69 (m, 2H), 7.62-7.54 (m, 2H), 7.52-7.42 (m, 3H), 7.13-7.03 (m, 2H), 6.35 (s, 1H), 4.66-4.58 (m, 1H), 3.74-3.67 (m, 2H), 3.25-3.20 (m, 2H), 3.19-3.14 (m, 2H), 3.12-3.04 (m, 2H), 2.49-2.37 (m, 6H), 2.32-2.19 (m, 3H), 1.97-1.90 (m, 4H).

### (R)-2-((4-fluorophenyl)amino)-2-oxo-1-phenylethyl 3-amino-6-(1-(1-(7-((1-(tert-butoxycarbonyl)piperidin-4-yl)methyl)-7-azaspiro[3.5]nonan-2-yl)piperidin-4-yl)-1H-pyrazol-4-yl)pyrazine-2-carboxylate (step 3)

(R)-2-((4-fluorophenyl)amino)-2-oxo-1-phenylethyl 6-(1-(1-(7-azaspiro[3.5]nonan-2-yl)piperidin-4-yl)-1H-pyrazol-4-yl)-3-aminopyrazine-2-carboxylate hydrochloride (104 mg, 0.15 mmol, 1 eq.) was dissolved in DCM (1.0 mL), and DIPEA (0.04 mL, 0.23 mmol, 1.5 eq.) was added at 0° C. After 10 minutes, tert-butyl 4-formylpiperidine-1-carboxylate (50 mg, 0.23 mmol, 1.5 eq.) was added to the above solution and reacted at room temperature for 4 hours. After adding NaBH(OAc)₃ (44 mg, 0.21 mmol, 1.34 eq.) at 0° C., the mixture was reacted overnight at room temperature. After completion of the reaction, the mixture was diluted with water and extracted with DCM. The combined organic layer was filtered after removing the residual water through anhydrous Na₂SO₄. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(7-((1-(tert-butoxycarbonyl)piperidin-4-yl)methyl)-7-azaspiro[3.5]nonan-2-yl)piperidin-4-yl)-1H-pyrazol-4-yl)pyrazine-2-carboxylate (104 mg, 83%).

¹H NMR (400 MHz, CDCl₃) δ 8.99 (s, 1H), 8.52 (s, 1H), 7.98 (s, 1H), 7.95 (s, 1H), 7.69-7.63 (m, 2H), 7.61-7.54 (m, 2H), 7.49-7.38 (m, 3H), 7.10-7.02 (m, 2H), 6.44 (s, 1H), 6.38 (s, 2H), 4.35-4.19 (m, 2H), 4.18-4.04 (m, 3H), 3.72-3.63 (m, 2H), 3.15-3.03 (m, 4H), 2.76-2.66 (m, 4H), 2.46-2.37 (m, 2H), 2.30-2.23 (m, 2H), 2.14-2.07 (m, 4H), 1.91-1.76 (m, 10H), 1.47 (s, 9H).

### (R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (step 4)

(R)-2-((4-fluorophenyl)amino)-2-oxo-1-phenylethyl 3-amino-6-(1-(1-(7-((1-(tert-butoxycarbonyl)piperidin-4-yl)methyl)-7-azaspiro[3.5]nonan-2-yl)piperidin-4-yl)-1H-pyrazol-4-yl)pyrazine-2-carboxylate (104 mg, 0.12 mmol) was dissolved in DCM (1.0 mL). Then, 4 N HCl in dioxane (0.5 mL) was slowly added, and reacted at room temperature for 1 hour. After completion of the reaction, the precipitate was filtered to obtain the desired (R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (80 mg, mixture).

### (R)-2-((4-fluorophenyl)amino)-2-oxo-1-phenylethyl 3-amino-6-(1-(1-(7-((1-(2-((2-(2,6-dioxopiperidin-3-yl)-1,3-dioxoisoindolin-5-yl)oxy)acetyl)piperidin-4-yl)methyl)-7-azaspiro[3.5]nonan-2-yl)piperidin-4-yl)-1H-pyrazol-4-yl)pyrazine-2-carboxylate [step 5, TDH-442]

(R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (15 mg, 0.020 mmol, 1 eq.), 2-((2-(2,6-dioxopiperidin-3-yl)-1,3-dioxoisoindolin-5-yl)oxy)acetic acid (7 mg, 0.020 mmol, 1.0 eq.), EDCI-HCl (5 mg, 0.022 mmol, 1.1 eq.), HOBt (4 mg, 0.022 mmol, 1.1 eq.) and DIPEA (0.02 mL, 0.10 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(7-((1-(2-((2-(2,6-dioxopiperidin-3-yl)-1,3-dioxoisoindolin-5-yl)oxy)acetyl)piperidin-4-yl)methyl)-7-azaspiro[3.5]nonan-2-yl)piperidin-4-yl)-1H-pyrazol-4-yl)pyrazine-2-carboxylate (9 mg, 45%).

### Synthesis of (R)-2-((4-fluorophenyl)amino)-2-oxo-1-phenylethyl 3-amino-6-(1-(1-(7-((1-((2-(2,6-dioxopiperidin-3-yl)-1,3-dioxoisoindolin-5-yl)glycyl)piperidin-4-yl)methyl)-7-azaspiro[3.5]nonan-2-yl)piperidin-4-yl)-1H-pyrazol-4-yl)pyrazine-2-carboxylate [TDH-443]

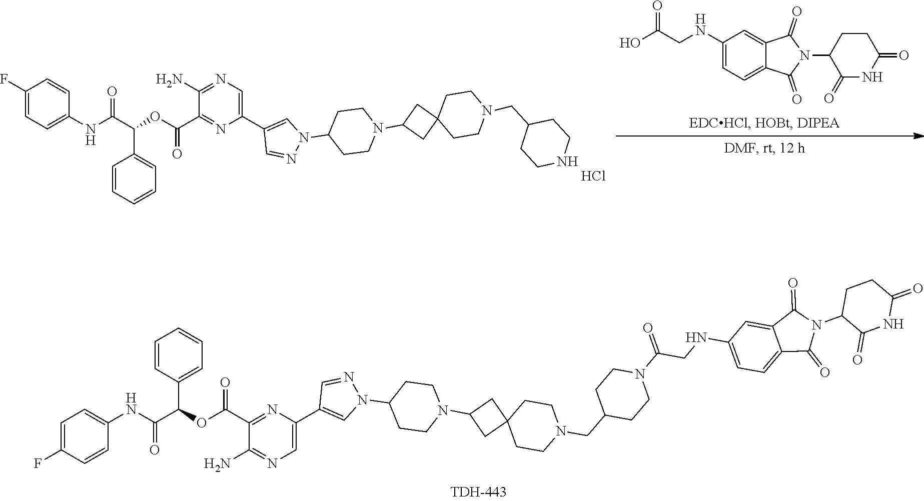 [SMILES: Nc1ncc(-c2cnn(C3CCN(C4CC5(CCN(CC6CCNCC6)CC5)C4)CC3)c2)nc1C(=O)O[C@@H](C(=O)Nc1ccc(F)cc1)c1ccccc1 ; Cl ; O=C(O)CNc1ccc2c(c1)C(=O)N(C1CCC(=O)NC1=O)C2=O ; Nc1ncc(-c2cnn(C3CCN(C4CC5(CCN(CC6CCN(C(=O)CNc7ccc8c(c7)C(=O)N(C7CCC(=O)NC7=O)C8=O)CC6)CC5)C4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (15 mg, 0.020 mmol, 1 eq.), (2-(2,6-dioxopiperidin-3-yl)-1,3-dioxoisoindolin-5-yl)glycine (7 mg, 0.020 mmol, 1.0 eq.), EDCI-HCl (5 mg, 0.022 mmol, 1.1 eq.), HOBt (4 mg, 0.022 mmol, 1.1 eq.) and DIPEA (0.02 mL, 0.10 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(7-((1-((2-(2,6-dioxopiperidin-3-yl)-1,3-dioxoisoindolin-5-yl)glycyl)piperidin-4-yl)methyl)-7-azaspiro[3.5]nonan-2-yl)piperidin-4-yl)-1H-pyrazol-4-yl)pyrazine-2-carboxylate (7 mg, 37%).

### Synthesis of (R)-2-((4-fluorophenyl)amino)-2-oxo-1-phenylethyl 3-amino-6-(1-(1-(7-((1-(2-(2,6-dioxopiperidin-3-yl)-1,3-dioxoisoindolin-5-yl)piperidin-4-yl)methyl)-7-azaspiro[3.5]nonan-2-yl)piperidin-4-yl)-1H-pyrazol-4-yl)pyrazine-2-carboxylate [TDH-472]

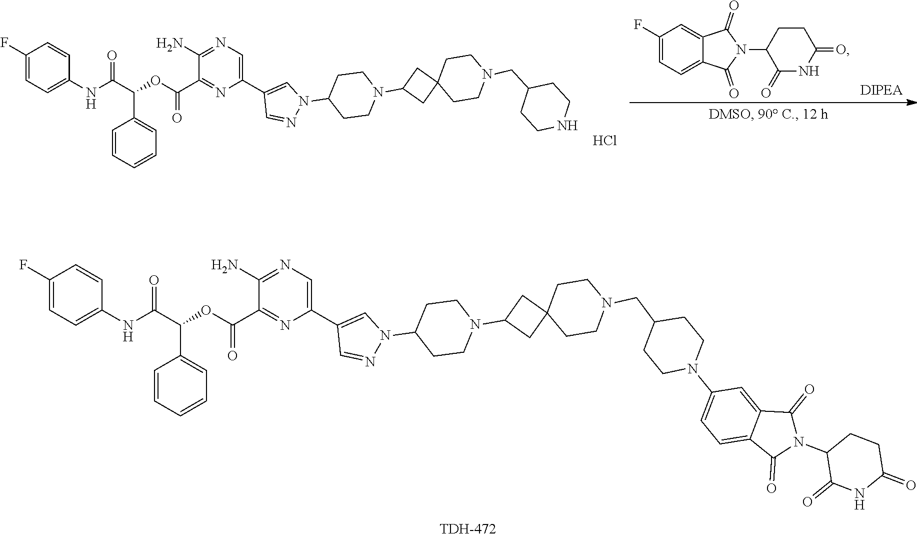 [SMILES: Nc1ncc(-c2cnn(C3CCN(C4CC5(CCN(CC6CCNCC6)CC5)C4)CC3)c2)nc1C(=O)O[C@@H](C(=O)Nc1ccc(F)cc1)c1ccccc1 ; Cl ; O=C1CCC(N2C(=O)c3ccc(F)cc3C2=O)C(=O)N1 ; Nc1ncc(-c2cnn(C3CCN(C4CC5(CCN(CC6CCN(c7ccc8c(c7)C(=O)N(C7CCC(=O)NC7=O)C8=O)CC6)CC5)C4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (20 mg, 0.026 mmol, 1 eq.), 2-(2,6-dioxopiperidin-3-yl)-5-fluoroisoindoline-1,3-dione (7 mg, 0.026 mmol, 1 eq.) and DIPEA (0.013 mL, 0.077 mmol, 3 eq.) were dissolved in DMSO (1.0 mL), and reacted at 90° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(7-((1-(2-(2,6-dioxopiperidin-3-yl)-1,3-dioxoisoindolin-5-yl)piperidin-4-yl)methyl)-7-azaspiro[3.5]nonan-2-yl)piperidin-4-yl)-1H-pyrazol-4-yl)pyrazine-2-carboxylate (9 mg, 38%).

### Synthesis of (R)-2-((4-fluorophenyl)amino)-2-oxo-1-phenylethyl 3-amino-6-(1-(1-(7-((1-(3-(2,4-dioxotetrahydropyrimidin-1(2H)-yl)-4-methoxybenzoyl)piperidin-4-yl)methyl)-7-azaspiro[3.5]nonan-2-yl)piperidin-4-yl)-1H-pyrazol-4-yl)pyrazine-2-carboxylate [TDH-499]

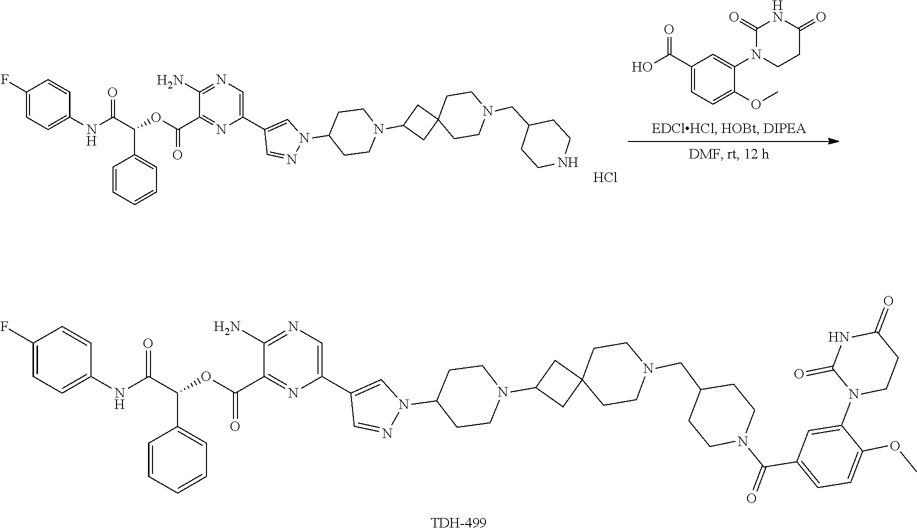 [SMILES: Nc1ncc(-c2cnn(C3CCN(C4CC5(CCN(CC6CCNCC6)CC5)C4)CC3)c2)nc1C(=O)O[C@@H](C(=O)Nc1ccc(F)cc1)c1ccccc1 ; Cl ; COc1ccc(C(=O)O)cc1N1CCC(=O)NC1=O ; COc1ccc(C(=O)N2CCC(CN3CCC4(CC3)CC(N3CCC(n5cc(-c6cnc(N)c(C(=O)O[C@@H](C(=O)Nc7ccc(F)cc7)c7ccccc7)n6)cn5)CC3)C4)CC2)cc1N1CCC(=O)NC1=O]

(R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (20 mg, 0.026 mmol, 1 eq.), 3-(2,4-dioxotetrahydropyrimidin-1(2H)-yl)-4-methoxybenzoic acid (7 mg, 0.026 mmol, 1.0 eq.), EDCI-HCl (5 mg, 0.028 mmol, 1.1 eq.), HOBt (4 mg, 0.028 mmol, 1.1 eq.), and DIPEA (0.02 mL, 0.13 mmol, 5 eq.) were dissolved in DMF (1.0 mL), and the mixture was reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(7-((1-(3-(2,4-dioxotetrahydropyrimidin-1(2H)-yl)-4-methoxybenzoyl)piperidin-4-yl)methyl)-7-azaspiro[3.5]nonan-2-yl) Piperidin-4-yl)-1H-pyrazol-4-yl)pyrazine-2-carboxylate (12 mg, 47%).

### Synthesis of (R)-2-((4-fluorophenyl)amino)-2-oxo-1-phenylethyl 3-amino-6-(1-(1-(7-((1-(2-(3-((1-(2,6-dioxopiperidin-3-yl)-2,5-dioxo-2,5-dihydro-1H-pyrrol-3-yl)amino)phenyl)acetyl)piperidin-4-yl)methyl)-7-azaspiro[3.5]nonan-2-yl)piperidin-4-yl)-1H-pyrazol-4-yl)pyrazine-2-carboxylate [TDH-500]

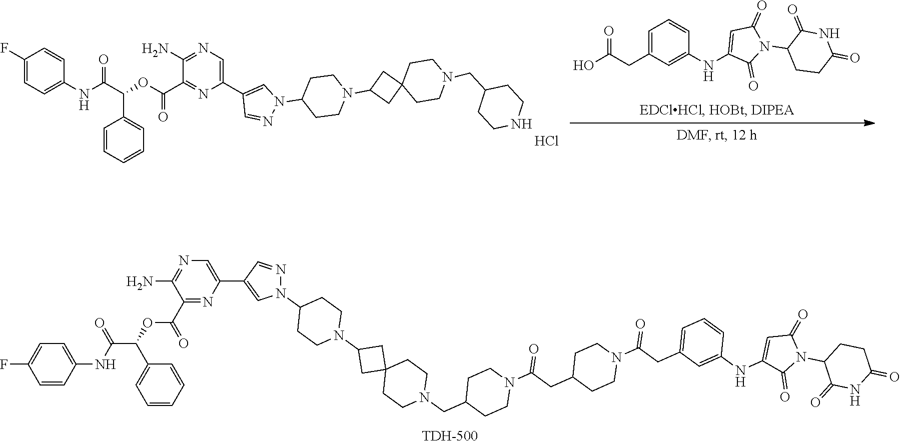 [SMILES: Nc1ncc(-c2cnn(C3CCN(C4CC5(CCN(CC6CCNCC6)CC5)C4)CC3)c2)nc1C(=O)O[C@@H](C(=O)Nc1ccc(F)cc1)c1ccccc1 ; Cl ; O=C(O)Cc1cccc(NC2=CC(=O)N(C3CCC(=O)NC3=O)C2=O)c1 ; Nc1ncc(-c2cnn(C3CCN(C4CC5(CCN(CC6CCN(C(=O)CC7CCN(C(=O)Cc8cccc(NC9=CC(=O)N(C%10CCC(=O)NC%10=O)C9=O)c8)CC7)CC6)CC5)C4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (20 mg, 0.026 mmol, 1 eq.), 2-(3-((1-(2,6-dioxopiperidin-3-yl)-2,5-dioxo-2,5-dihydro-1H-pyrrol-3-yl)amino)phenyl)acetic acid (9 mg, 0.026 mmol, 1.0 eq.), EDCI-HCl (5 mg, 0.028 mmol, 1.1 eq.), HOBt (4 mg, 0.028 mmol, 1.1 eq.), and DIPEA (0.02 mL, 0.13 mmol, 5 eq.) were dissolved in DMF (1.0 mL), and reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(7-((1-(2-(3-((1-(2,6-dioxopiperidin-3-yl)-2,5-dioxo-2,5-dihydro-1H-pyrrol-3-yl)amino)phenyl)acetyl)piperidin-4-yl)methyl)-7-azaspiro[3.5]nonan-2-yl)piperidin-4-yl)-1H-pyrazol-4-yl)pyrazine-2-carboxylate (8 mg, 29%).

### Synthesis of (R)-2-((4-fluorophenyl)amino)-2-oxo-1-phenylethyl 3-amino-6-(1-(1-(7-((1-(1-(1-(2,6-dioxopiperidin-3-yl)-2,5-dioxo-2,5-dihydro-1H-pyrrol-3-yl)piperidine-4-carbonyl)piperidin-4-yl)methyl)-7-azaspiro[3.5]nonan-2-yl)piperidin-4-yl)-1H-pyrazol-4-yl)pyrazine-2-carboxylate [TDH-528]

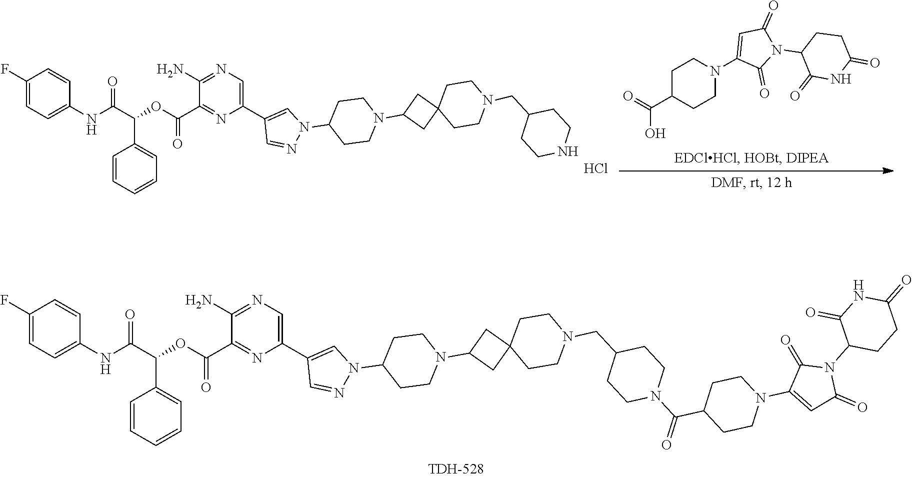 [SMILES: Nc1ncc(-c2cnn(C3CCN(C4CC5(CCN(CC6CCNCC6)CC5)C4)CC3)c2)nc1C(=O)O[C@@H](C(=O)Nc1ccc(F)cc1)c1ccccc1 ; Cl ; O=C1CCC(N2C(=O)C=C(N3CCC(C(=O)O)CC3)C2=O)C(=O)N1 ; Nc1ncc(-c2cnn(C3CCN(C4CC5(CCN(CC6CCN(C(=O)C7CCN(C8=CC(=O)N(C9CCC(=O)NC9=O)C8=O)CC7)CC6)CC5)C4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (20 mg, 0.026 mmol, 1 eq.), 1-(1-(2,6-dioxopiperidin-3-yl)-2,5-dioxo-2,5-dihydro-1H-pyrrol-3-yl)piperidine-4-carboxylic acid (9 mg, 0.026 mmol, 1.0 eq.), EDCI-HCl (5 mg, 0.028 mmol, 1.1 eq.), HOBt (4 mg, 0.028 mmol, 1.1 eq.), and DIPEA (0.02 mL, 0.13 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(7-((1-(1-(1-(2,6-dioxopiperidin-3-yl)-2,5-dioxo-2,5-dihydro-1H-pyrrol-3-yl)piperidine-4-carbonyl)piperidin-4-yl)methyl)-7-azaspiro[3.5]nonan-2-yl)piperidin-4-yl)-1H-pyrazol-4-yl)pyrazine-2-carboxylate (6 mg, 18%).

### Synthesis of (R)-2-((4-fluorophenyl)amino)-2-oxo-1-phenylethyl 3-amino-6-(1-(1-(7-((1-(1-(1-(2,6-dioxopiperidin-3-yl)-2,5-dioxo-2,5-dihydro-1H-pyrrol-3-yl)azetidine-3-carbonyl)piperidin-4-yl)methyl)-7-azaspiro[3.5]nonan-2-yl)piperidin-4-yl)-1H-pyrazol-4-yl)pyrazine-2-carboxylate [TDH-534]

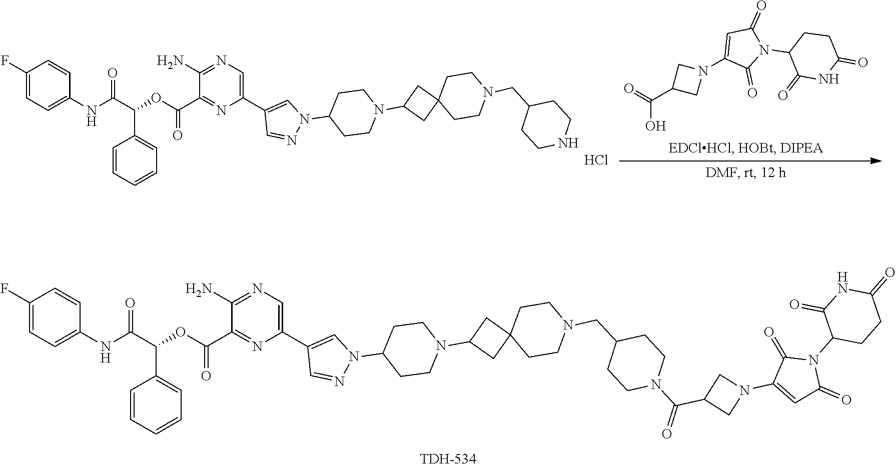 [SMILES: Nc1ncc(-c2cnn(C3CCN(C4CC5(CCN(CC6CCNCC6)CC5)C4)CC3)c2)nc1C(=O)O[C@@H](C(=O)Nc1ccc(F)cc1)c1ccccc1 ; Cl ; O=C1CCC(N2C(=O)C=C(N3CC(C(=O)O)C3)C2=O)C(=O)N1 ; Nc1ncc(-c2cnn(C3CCN(C4CC5(CCN(CC6CCN(C(=O)C7CN(C8=CC(=O)N(C9CCC(=O)NC9=O)C8=O)C7)CC6)CC5)C4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (20 mg, 0.026 mmol, 1 eq.), 1-(1-(2,6-dioxopiperidin-3-yl)-2,5-dioxo-2,5-dihydro-1H-pyrrol-3-yl)azetidine-3-carboxylic acid (9 mg, 0.026 mmol, 1.0 eq.), EDCI-HCl (5 mg, 0.028 mmol, 1.1 eq.), HOBt (4 mg, 0.028 mmol, 1.1 eq.), and DIPEA (0.02 mL, 0.13 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(7-((1-(1-(1-(2,6-dioxopiperidin-3-yl)-2,5-dioxo-2,5-dihydro-1H-pyrrol-3-yl)azetidine-3-carbonyl)piperidin-4-yl)methyl)-7-azaspiro[3.5]nonan-2-yl)piperidin-4-yl)-1H-pyrazol-4-yl)pyrazine-2-carboxylate (2 mg, 9%).

### Synthesis of (R)-2-((4-fluorophenyl)amino)-2-oxo-1-phenylethyl 3-amino-6-(1-(1-(7-((1-(4-((2,6-dioxopiperidin-3-yl)carbamoyl)benzoyl)piperidin-4-yl)methyl)-7-azaspiro[3.5]nonan-2-yl)piperidin-4-yl)-1H-pyrazol-4-yl)pyrazine-2-carboxylate [TDH-537]

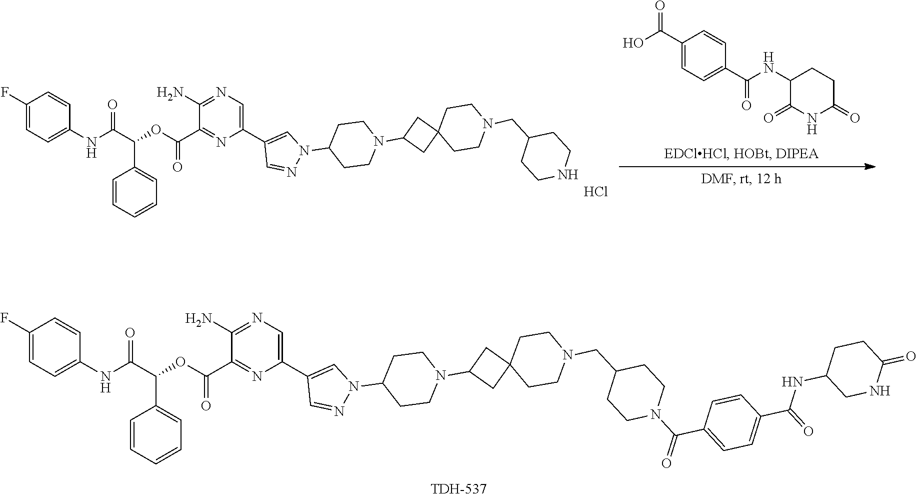 [SMILES: Nc1ncc(-c2cnn(C3CCN(C4CC5(CCN(CC6CCNCC6)CC5)C4)CC3)c2)nc1C(=O)O[C@@H](C(=O)Nc1ccc(F)cc1)c1ccccc1 ; Cl ; O=C1CCC(NC(=O)c2ccc(C(=O)O)cc2)C(=O)N1 ; Nc1ncc(-c2cnn(C3CCN(C4CC5(CCN(CC6CCN(C(=O)c7ccc(C(=O)NC8CCC(=O)NC8)cc7)CC6)CC5)C4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (20 mg, 0.026 mmol, 1 eq.), 4-((2,6-dioxopiperidin-3-yl)carbamoyl)benzoic acid (8 mg, 0.026 mmol, 1.0 eq.), EDCI-HCl (5 mg, 0.028 mmol, 1.1 eq.), HOBt (4 mg, 0.028 mmol, 1.1 eq.), and DIPEA (0.02 mL, 0.13 mmol, 5 eq.) were dissolved in DMF (1.0 mL) and reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(7-((1-(4-((2,6-dioxopiperidin-3-yl)carbamoyl)benzoyl)piperidin-4-yl)methyl)-7-azaspiro[3.5]nonan-2-yl)piperidin-4-yl)-1H-pyrazol-4-yl)pyrazine-2-carboxylate (15 mg, 56%).

### Synthesis of (R)-2-((4-fluorophenyl)amino)-2-oxo-1-phenylethyl 3-amino-6-(1-(1-(7-((1-(3-((2,6-dioxopiperidin-3-yl)carbamoyl)benzoyl)piperidin-4-yl)methyl)-7-azaspiro[3.5]nonan-2-yl)piperidin-4-yl)-1H-pyrazol-4-yl)pyrazine-2-carboxylate[TDH-544]

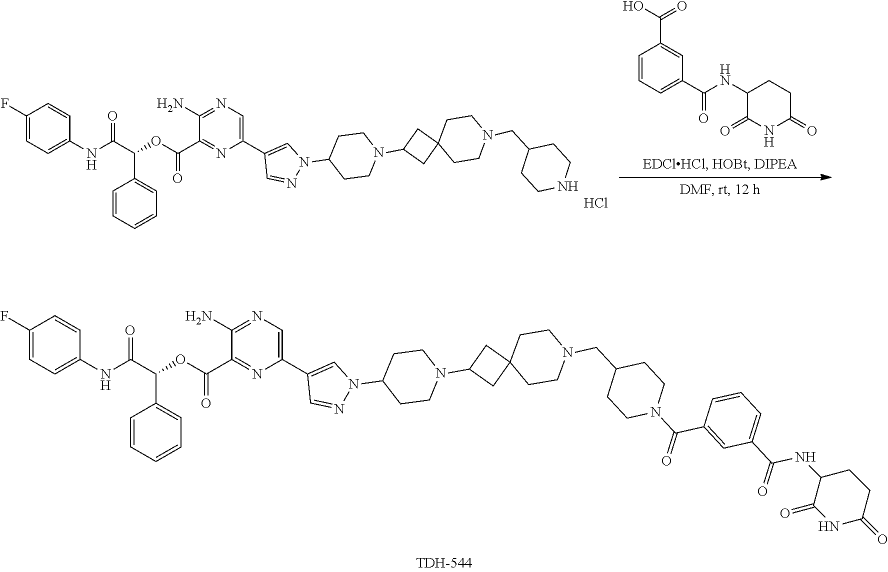 [SMILES: Nc1ncc(-c2cnn(C3CCN(C4CC5(CCN(CC6CCNCC6)CC5)C4)CC3)c2)nc1C(=O)O[C@@H](C(=O)Nc1ccc(F)cc1)c1ccccc1 ; Cl ; O=C1CCC(NC(=O)c2cccc(C(=O)O)c2)C(=O)N1 ; Nc1ncc(-c2cnn(C3CCN(C4CC5(CCN(CC6CCN(C(=O)c7cccc(C(=O)NC8CCC(=O)NC8=O)c7)CC6)CC5)C4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (20 mg, 0.026 mmol, 1 eq.), 3-((2,6-dioxopiperidin-3-yl)carbamoyl)benzoic acid (8 mg, 0.026 mmol, 1.0 eq.), EDCI-HCl (5 mg, 0.028 mmol, 1.1 eq.), HOBt (4 mg, 0.028 mmol, 1.1 eq.), and DIPEA (0.02 mL, 0.13 mmol, 5 eq.) were dissolved in DMF (1.0 mL) and reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(7-((1-(3-((2,6-dioxopiperidin-3-yl)carbamoyl)benzoyl)piperidin-4-yl)methyl)-7-azaspiro[3.5]nonan-2-yl)piperidin-4-yl)-1H-pyrazol-4-yl)pyrazine-2-carboxylate (9 mg, 34%).

### Synthesis of (R)-2-((4-fluorophenyl)amino)-2-oxo-1-phenylethyl 3-amino-6-(1-(1-(7-((1-(2-(2,6-dioxopiperidin-3-yl)-1-oxoisoindoline-5-carbonyl)piperidin-4-yl)methyl)-7-azaspiro[3.5]nonan-2-yl)piperidin-4-yl)-1H-pyrazol-4-yl)pyrazine-2-carboxylate [TDH-548]

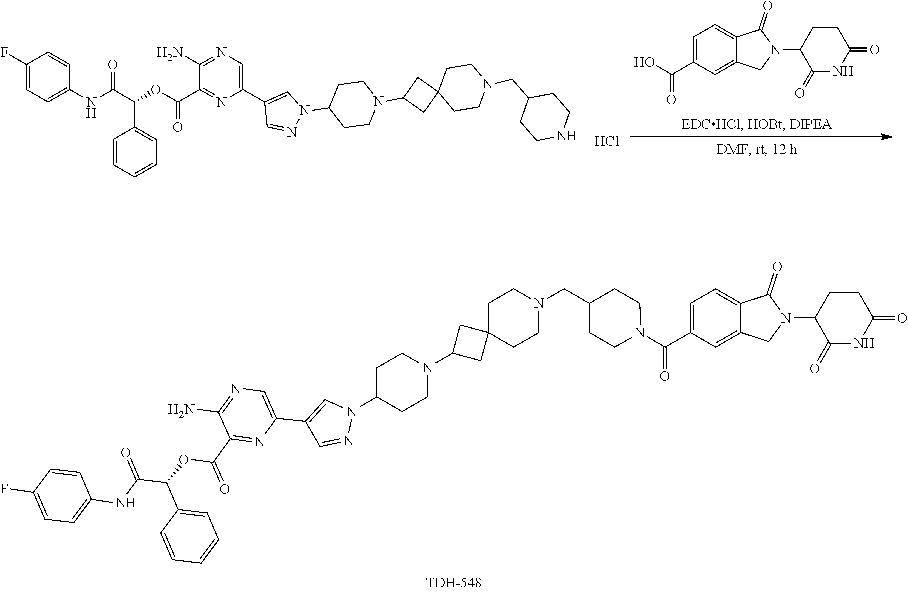 [SMILES: Nc1ncc(-c2cnn(C3CCN(C4CC5(CCN(CC6CCNCC6)CC5)C4)CC3)c2)nc1C(=O)O[C@@H](C(=O)Nc1ccc(F)cc1)c1ccccc1 ; Cl ; O=C1CCC(N2Cc3cc(C(=O)O)ccc3C2=O)C(=O)N1 ; Nc1ncc(-c2cnn(C3CCN(C4CC5(CCN(CC6CCN(C(=O)c7ccc8c(c7)CN(C7CCC(=O)NC7=O)C8=O)CC6)CC5)C4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (20 mg, 0.026 mmol, 1 eq.), 2-(2,6-dioxopiperidin-3-yl)-1-oxoisoindoline-5-carboxylic acid (8 mg, 0.026 mmol, 1.0 eq.), EDCI-HCl (5 mg, 0.028 mmol, 1.1 eq.), HOBt (4 mg, 0.028 mmol, 1.1 eq.) and DIPEA (0.02 mL, 0.13 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(7-((1-(2-(2,6-dioxopiperidin-3-yl)-1-oxoisoindoline-5-carbonyl)piperidin-4-yl)methyl)-7-azaspiro[3.5]nonan-2-yl)piperidin-4-yl)-1H-pyrazol-4-yl)pyrazine-2-carboxylate (16 mg, 59%).

### Synthesis of (R)-2-((4-fluorophenyl)amino)-2-oxo-1-phenylethyl 3-amino-6-(1-(1-(7-((1-(3-(2,4-dioxotetrahydropyrimidin-1(2H)-yl)benzofuran-6-carbonyl)piperidin-4-yl)methyl)-7-azaspiro[3.5]nonan-2-yl)piperidin-4-yl)-1H-pyrazol-4-yl)pyrazine-2-carboxylate [TDH-553]

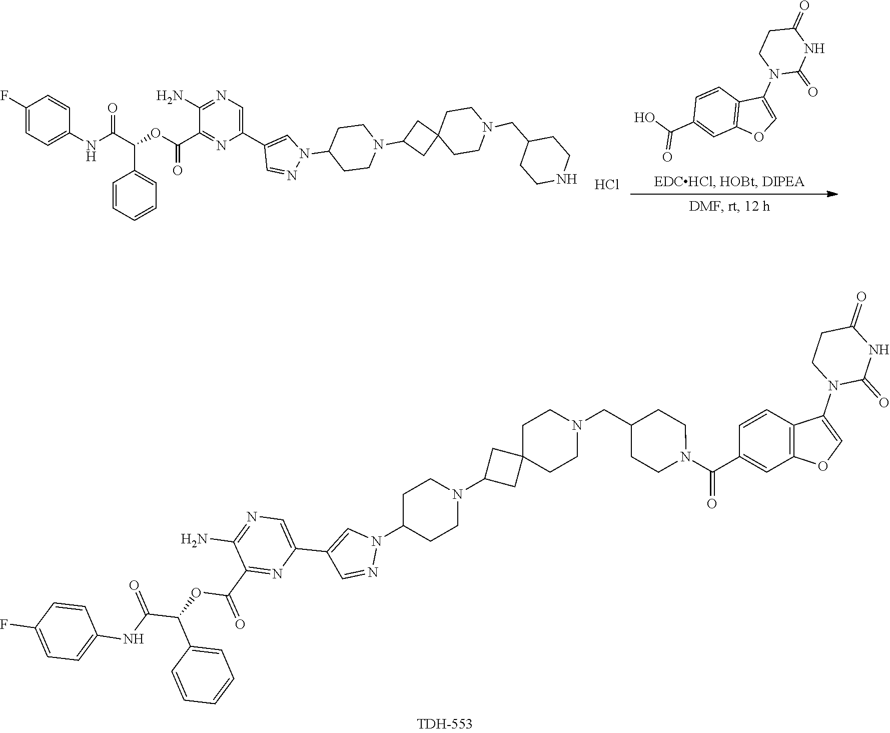 [SMILES: Nc1ncc(-c2cnn(C3CCN(C4CC5(CCN(CC6CCNCC6)CC5)C4)CC3)c2)nc1C(=O)O[C@@H](C(=O)Nc1ccc(F)cc1)c1ccccc1 ; Cl ; O=C1CCN(c2coc3cc(C(=O)O)ccc23)C(=O)N1 ; Nc1ncc(-c2cnn(C3CCN(C4CC5(CCN(CC6CCN(C(=O)c7ccc8c(N9CCC(=O)NC9=O)coc8c7)CC6)CC5)C4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(7-(piperidin-4-ylmethyl)-7-azaspiro[3.5]nonan-2-yl)piperidin-4-yl)-1H-pyrazol-4-yl)pyrazine-2-carboxylate hydrochloride (20 mg, 0.026 mmol, 1 eq.), 3-(2,4-dioxotetrahydropyrimidin-1(2H)-yl)benzofuran-6-carboxylic acid (8 mg, 0.026 mmol, 1.0 eq.), EDCI-HCl (5 mg, 0.028 mmol, 1.1 eq.), HOBt (4 mg, 0.028 mmol, 1.1 eq.) and DIPEA (0.02 mL, 0.13 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(7-((1-(3-(2,4-dioxotetrahydropyrimidin-1(2H)-yl)benzofuran-6-carbonyl)piperidin-4-yl)methyl)-7-azaspiro[3.5]nonan-2-yl)piperidin-4-yl)-1H-pyrazol-4-yl)pyrazine-2-carboxylate (15 mg, 56%).

### Synthesis of (R)-2-((4-fluorophenyl)amino)-2-oxo-1-phenylethyl 3-amino-6-(1-(1-(2-(1-(3-(2,4-dioxotetrahydropyrimidin-1(2H)-yl)-4-methoxybenzoyl)piperidin-4-yl)ethyl)piperidin-4-yl)-1H-pyrazol-4-yl)pyrazine-2-carboxylate [TDH-446]

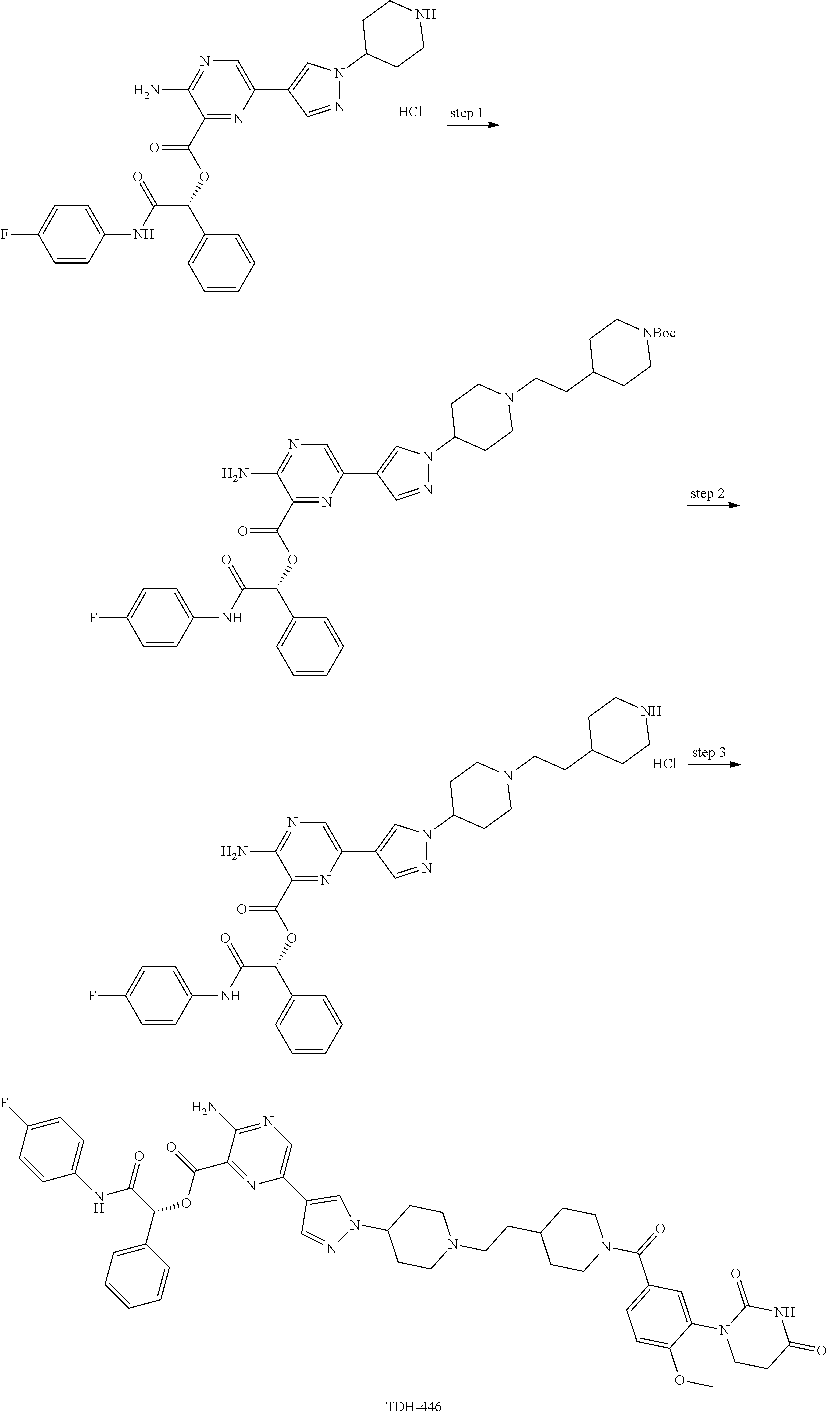 [SMILES: Nc1ncc(-c2cnn(C3CCNCC3)c2)nc1C(=O)O[C@@H](C(=O)Nc1ccc(F)cc1)c1ccccc1 ; Cl ; CC(C)(C)OC(=O)N1CCC(CCN2CCC(n3cc(-c4cnc(N)c(C(=O)O[C@@H](C(=O)Nc5ccc(F)cc5)c5ccccc5)n4)cn3)CC2)CC1 ; Nc1ncc(-c2cnn(C3CCN(CCC4CCNCC4)CC3)c2)nc1C(=O)O[C@@H](C(=O)Nc1ccc(F)cc1)c1ccccc1 ; Cl ; COc1ccc(C(=O)N2CCC(CCN3CCC(n4cc(-c5cnc(N)c(C(=O)O[C@@H](C(=O)Nc6ccc(F)cc6)c6ccccc6)n5)cn4)CC3)CC2)cc1N1CCC(=O)NC1=O]

### (R)-2-((4-fluorophenyl)amino)-2-oxo-1-phenylethyl 3-amino-6-(1-(1-(2-(1-(tert-butoxycarbonyl)piperidin-4-yl)ethyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylate hydrochloride (300 mg, 0.54 mmol, 1 eq.) was dissolved in DCM (3.0 mL), and DIPEA (0.15 mL, 0.81 mmol, 1.5 eq.) was added at 0° C. After 10 minutes, tert-butyl 4-(2-oxoethyl)piperidine-1-carboxylate (180 mg, 0.81 mmol, 1.5 eq.) was added to the above solution and reacted at room temperature for 4 hours. After adding NaBH(OAc)₃ (150 mg, 0.78 mmol, 1.34 eq.) at 0° C., the mixture was reacted overnight at room temperature. After completion of the reaction, the mixture was diluted with water and extracted with DCM. The combined organic layer was filtered after removing the residual water through anhydrous Na₂SO₄. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(tert-butoxycarbonyl)piperidin-4-yl)ethyl)piperidin-4-yl)-1H-pyrazol-4-yl)pyrazine-2-carboxylate (433 mg, mixture).

### (R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-(2-(1-(tert-butoxycarbonyl)piperidin-4-yl)ethyl)piperidin-4-yl)-1H-pyrazol-4-yl)pyrazine-2-carboxylate (433 mg, 0.60 mmol) was dissolved in DCM (2.0 mL) and 4 N HCl in dioxane (1.0 mL) was slowly added. After that, it was reacted at room temperature for 1 hour. After completion of the reaction, the precipitate was filtered to obtain (R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (352 mg, mixture).

### (R)-2-((4-fluorophenyl)amino)-2-oxo-1-phenylethyl 3-amino-6-(1-(1-(2-(1-(3-(2,4-dioxotetrahydropyrimidin-1(2H)-yl)-4-methoxybenzoyl)piperidin-4-yl)ethyl)piperidin-4-yl)-1H-pyrazol-4-yl)pyrazine-2-carboxylate [step 3, TDH-446]

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (20 mg, 0.030 mmol, 1 eq.), 3-(2,4-dioxotetrahydropyrimidin-1(2H)-yl)-4-methoxybenzoic acid (8 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(3-(2,4-dioxotetrahydropyrimidin-1(2H)-yl)-4-methoxybenzoyl)piperidin-4-yl)ethyl)piperidin-4-yl)-1H-pyrazol-4-yl) pyrazine-2-carboxylate (19 mg, 74%).

### Synthesis of (R)-2-((4-fluorophenyl)amino)-2-oxo-1-phenylethyl 3-amino-6-(1-(1-(2-(1-(3-(2-(2,6-dioxopiperidin-3-yl)-1-oxoisoindolin-5-yl)propanoyl)piperidin-4-yl)ethyl)piperidin-4-yl)-1H-pyrazol-4-yl)pyrazine-2-carboxylate [TDH-475]

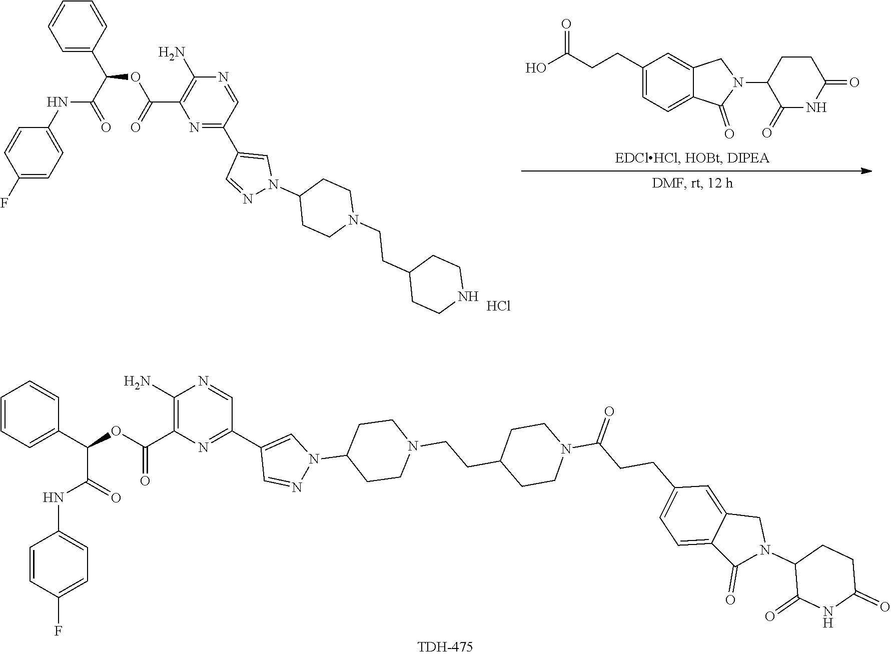 [SMILES: Nc1ncc(-c2cnn(C3CCN(CCC4CCNCC4)CC3)c2)nc1C(=O)O[C@@H](C(=O)Nc1ccc(F)cc1)c1ccccc1 ; Cl ; O=C(O)CCc1ccc2c(c1)CN(C1CCC(=O)NC1=O)C2=O ; Nc1ncc(-c2cnn(C3CCN(CCC4CCN(C(=O)CCc5ccc6c(c5)CN(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (20 mg, 0.030 mmol, 1 eq.), 3-(2-(2,6-dioxopiperidin-3-yl)-1-oxoisoindolin-5-yl)propanoic acid (10 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(3-(2-(2,6-dioxopiperidin-3-yl)-1-oxoisoindolin-5-yl)propanoyl)piperidin-4-yl)ethyl)piperidin-4-yl)-1H-pyrazol-4-yl)pyrazine-2-carboxylate (12 mg, 43%).

### Synthesis of (R)-2-((4-fluorophenyl)amino)-2-oxo-1-phenylethyl 3-amino-6-(1-(1-(2-(1-(1-(3-(2,4-dioxotetrahydropyrimidin-1(2H)-yl)-4-methoxybenzoyl)piperidine-4-carbonyl)piperidin-4-yl)ethyl)piperidin-4-yl)-1H-pyrazol-4-yl)pyrazine-2-carboxylate [TDH-517]

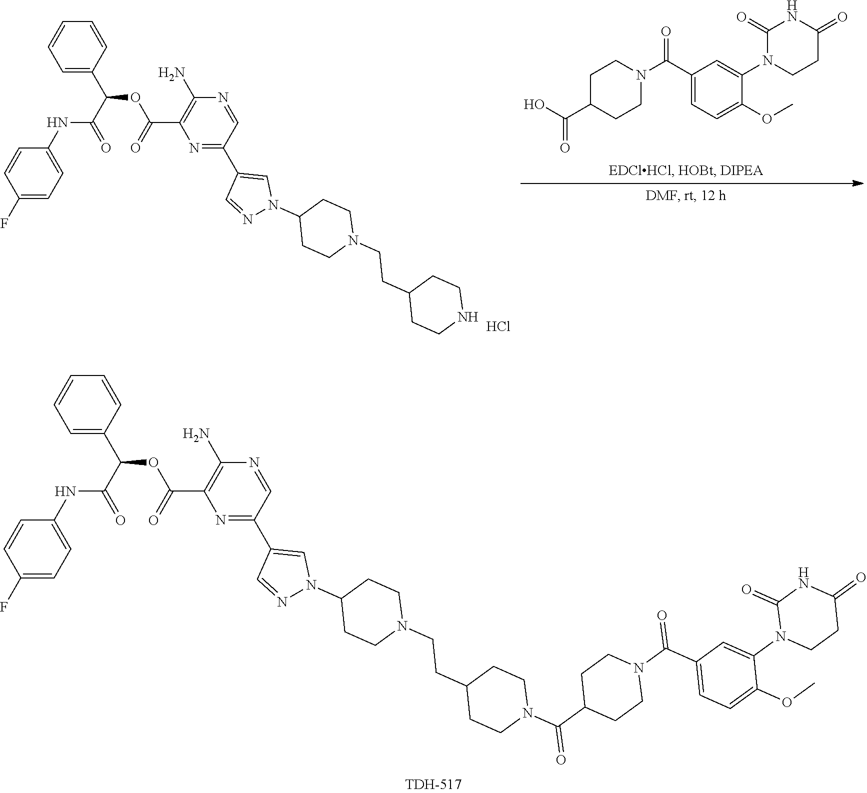 [SMILES: Nc1ncc(-c2cnn(C3CCN(CCC4CCNCC4)CC3)c2)nc1C(=O)O[C@@H](C(=O)Nc1ccc(F)cc1)c1ccccc1 ; Cl ; COc1ccc(C(=O)N2CCC(C(=O)O)CC2)cc1N1CCC(=O)NC1=O ; COc1ccc(C(=O)N2CCC(C(=O)N3CCC(CCN4CCC(n5cc(-c6cnc(N)c(C(=O)O[C@@H](C(=O)Nc7ccc(F)cc7)c7ccccc7)n6)cn5)CC4)CC3)CC2)cc1N1CCC(=O)NC1=O]

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (20 mg, 0.030 mmol, 1 eq.), 1-(3-(2,4-dioxotetrahydropyrimidin-1(2H)-yl)-4-methoxybenzoyl)piperidine-4-carboxylic acid (11 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL) and the mixture was reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(1-(3-(2,4-dioxotetrahydropyrimidin-1(2H)-yl)-4-methoxybenzoyl)piperidine-4-carbonyl)piperidin-4-yl)ethyl)piperidin-4-yl)-1H-pyrazol-4-yl)pyrazine-2-carboxylate (4 mg, 16%).

### Synthesis of (R)-2-((4-fluorophenyl)amino)-2-oxo-1-phenylethyl 3-amino-6-(1-(1-(2-(1-(2-(3-((1-(2,6-dioxopiperidin-3-yl)-2,5-dioxo-2,5-dihydro-1H-pyrrol-3-yl)amino)phenyl)acetyl)piperidin-4-yl)ethyl)piperidin-4-yl)-1H-pyrazol-4-yl)pyrazine-2-carboxylate [TDH-518]

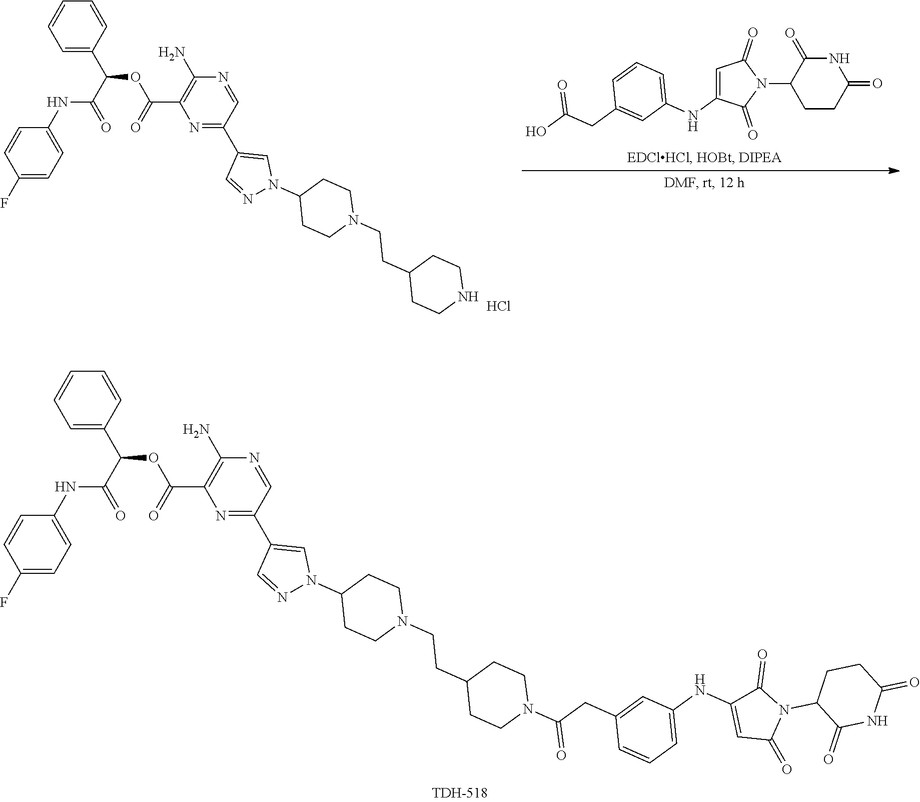 [SMILES: Nc1ncc(-c2cnn(C3CCN(CCC4CCNCC4)CC3)c2)nc1C(=O)O[C@@H](C(=O)Nc1ccc(F)cc1)c1ccccc1 ; Cl ; O=C(O)Cc1cccc(NC2=CC(=O)N(C3CCC(=O)NC3=O)C2=O)c1 ; Nc1ncc(-c2cnn(C3CCN(CCC4CCN(C(=O)Cc5cccc(NC6=CC(=O)N(C7CCC(=O)NC7=O)C6=O)c5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (20 mg, 0.030 mmol, 1 eq.), 2-(3-((1-(2,6-dioxopiperidin-3-yl)-2,5-dioxo-2,5-dihydro-1H-pyrrol-3-yl)amino)phenyl)acetic acid (11 mg, 0.030 mmol, 1.0 eq.), EDCI-HCl (6 mg, 0.033 mmol, 1.1 eq.).), HOBt (5 mg, 0.033 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(2-(3-((1-(2,6-dioxopiperidin-3-yl)-2,5-dioxo-2,5-dihydro-1H-pyrrol-3-yl)amino) phenyl)acetyl)piperidin-4-yl)ethyl)piperidin-4-yl)-1H-pyrazol-4-yl)pyrazine-2-carboxylate (13 mg, 45%).

### Synthesis of (R)-2-((4-fluorophenyl)amino)-2-oxo-1-phenylethyl 3-amino-6-(1-(1-(2-(1-(2-(2,6-dioxopiperidin-3-yl)-6-fluoro-1,3-dioxoisoindolin-5-yl)piperidin-4-yl)ethyl)piperidin-4-yl)-1H-pyrazol-4-yl)pyrazine-2-carboxylate [TDH-520]

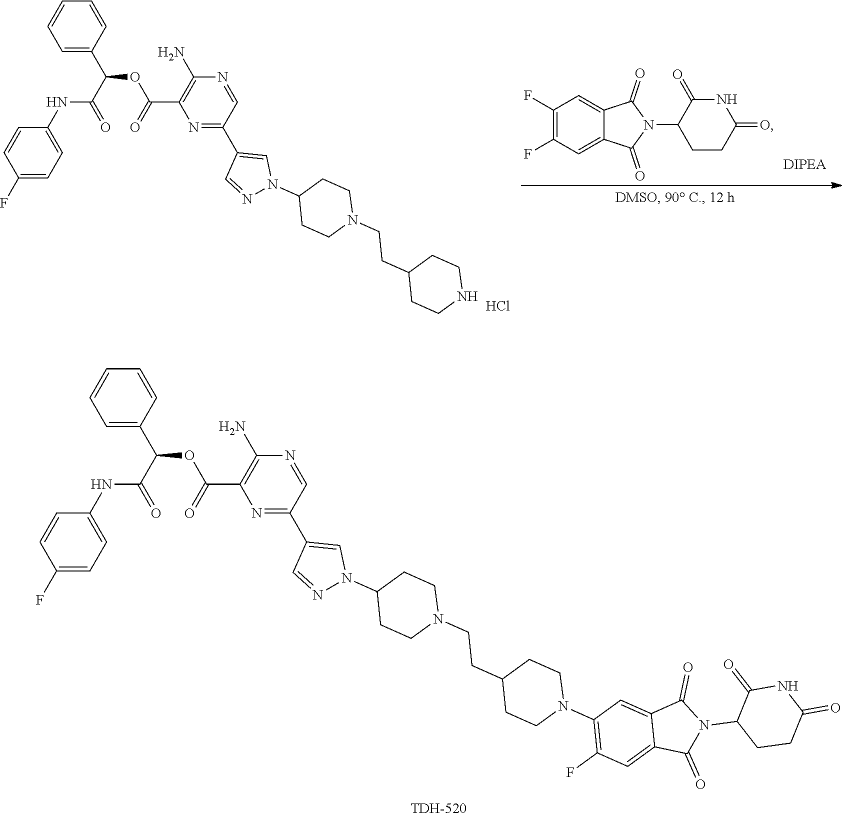 [SMILES: Nc1ncc(-c2cnn(C3CCN(CCC4CCNCC4)CC3)c2)nc1C(=O)O[C@@H](C(=O)Nc1ccc(F)cc1)c1ccccc1 ; Cl ; O=C1CCC(N2C(=O)c3cc(F)c(F)cc3C2=O)C(=O)N1 ; Nc1ncc(-c2cnn(C3CCN(CCC4CCN(c5cc6c(cc5F)C(=O)N(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (20 mg, 0.030 mmol, 1 eq.), 2-(2,6-dioxopiperidin-3-yl)-5,6-difluoroisoindoline-1,3-dione (9 mg, 0.029 mmol, 0.95 eq.) and DIPEA (0.015 mL, 0.089 mmol, 3 eq.) were dissolved in DMSO (1.0 mL), and the mixture was reacted at 90° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(2-(2,6-dioxopiperidin-3-yl)-6-fluoro-1,3-dioxoisoindolin-5-yl)piperidin-4-yl)ethyl)piperidin-4-yl)-1H-pyrazol-4-yl)pyrazine-2-carboxylate (11 mg, 41%).

### Synthesis of (R)-2-((4-fluorophenyl)amino)-2-oxo-1-phenylethyl 3-amino-6-(1-(1-(2-(1-(1-(2,6-dioxopiperidin-3-yl)-2,5-dioxo-2,5-dihydro-1H-pyrrol-3-yl)piperidin-4-yl)ethyl)piperidin-4-yl)-1H-pyrazol-4-yl)pyrazine-2-carboxylate [TDH-522]

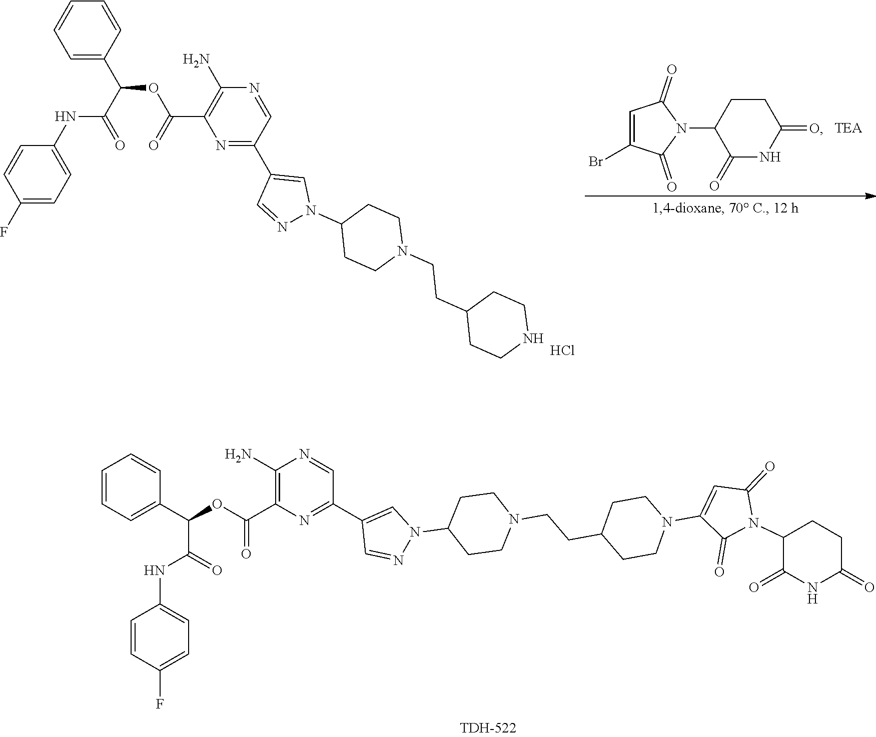 [SMILES: Nc1ncc(-c2cnn(C3CCN(CCC4CCNCC4)CC3)c2)nc1C(=O)O[C@@H](C(=O)Nc1ccc(F)cc1)c1ccccc1 ; Cl ; O=C1CCC(N2C(=O)C=C(Br)C2=O)C(=O)N1 ; Nc1ncc(-c2cnn(C3CCN(CCC4CCN(C5=CC(=O)N(C6CCC(=O)NC6=O)C5=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (73 mg, 0.11 mmol, 1.5 eq.), 3-(3-bromo-2,5-dioxo-2,5-dihydro-1H-pyrrol-1-yl)piperidine-2,6-dione (20 mg, 0.07 mmol, 1.0 eq.) and TEA (0.03 mL, 0.21 mmol, 3 eq.) were dissolved in 1,4-dioxane (1.0 mL), and reacted overnight at 70° C. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(1-(2,6-dioxopiperidin-3-yl)-2,5-dioxo-2,5-dihydro-1H-pyrrol-3-yl)piperidin-4-yl)ethyl)piperidin-4-yl)-1H-pyrazol-4-yl)pyrazine-2-carboxylate (7 mg, 13%).

### Synthesis of (R)-2-((4-fluorophenyl)amino)-2-oxo-1-phenylethyl 3-amino-6-(1-(1-(2-(1-(1-(1-(2,6-dioxopiperidin-3-yl)-2,5-dioxo-2,5-dihydro-1H-pyrrol-3-yl)piperidine-4-carbonyl)piperidin-4-yl)ethyl)piperidin-4-yl)-1H-pyrazol-4-yl)pyrazine-2-carboxylate [TDH-524]

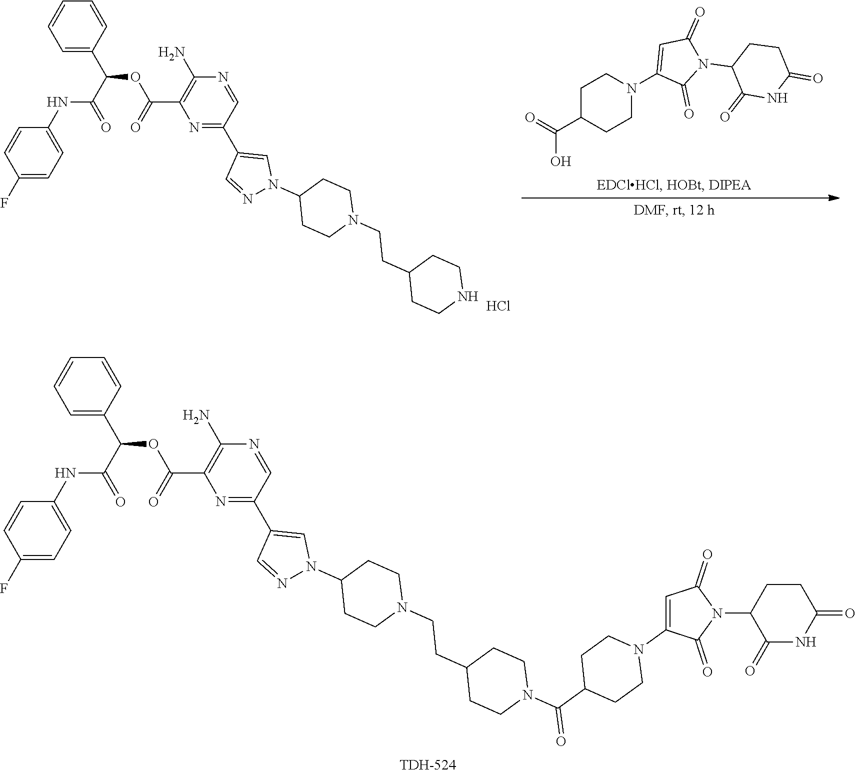 [SMILES: Nc1ncc(-c2cnn(C3CCN(CCC4CCNCC4)CC3)c2)nc1C(=O)O[C@@H](C(=O)Nc1ccc(F)cc1)c1ccccc1 ; Cl ; O=C1CCC(N2C(=O)C=C(N3CCC(C(=O)O)CC3)C2=O)C(=O)N1 ; Nc1ncc(-c2cnn(C3CCN(CCC4CCN(C(=O)C5CCN(C6=CC(=O)N(C7CCC(=O)NC7=O)C6=O)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (20 mg, 0.030 mmol, 1 eq.), 1-(1-(2,6-dioxopiperidin-3-yl)-2,5-dioxo-2,5-dihydro-1H-pyrrol-3-yl)piperidine-4-carboxylic acid (11 mg, 0.030 mmol, 1.0 eq.), EDCI-HCl (6 mg, 0.033 mmol, 1.1 eq.), HOBt (5 mg, 0.033 mmol, 1.1 eq.) and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(1-(1-(2,6-dioxopiperidin-3-yl)-2,5-dioxo-2,5-dihydro-1H-pyrrol-3-yl)piperidine-4-carbonyl)piperidin-4-yl)ethyl)piperidin-4-yl)-1H-pyrazol-4-yl)pyrazine-2-carboxylate (9 mg, 32%).

### Synthesis of (R)-2-((4-fluorophenyl)amino)-2-oxo-1-phenylethyl 3-amino-6-(1-(1-(2-(1-(1-(1-(2,6-dioxopiperidin-3-yl)-2,5-dioxo-2,5-dihydro-1H-pyrrol-3-yl)azetidine-3-carbonyl)piperidin-4-yl)ethyl)piperidin-4-yl)-1H-pyrazol-4-yl)pyrazine-2-carboxylate [TDH-531]

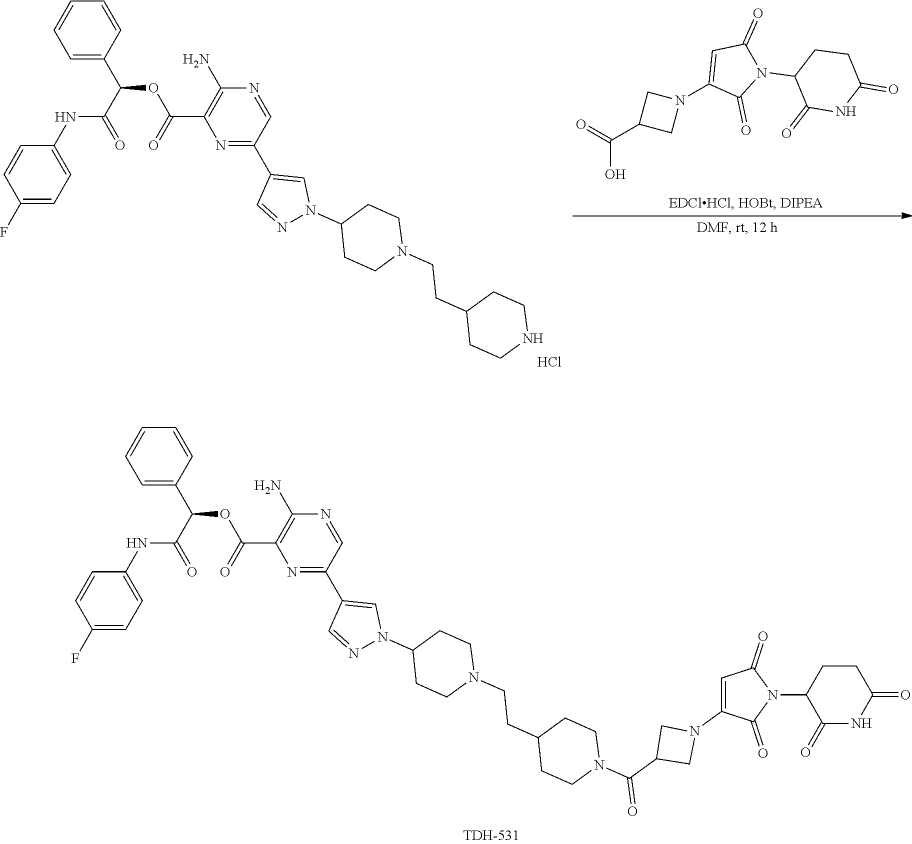 [SMILES: Nc1ncc(-c2cnn(C3CCN(CCC4CCNCC4)CC3)c2)nc1C(=O)O[C@@H](C(=O)Nc1ccc(F)cc1)c1ccccc1 ; O=C1CCC(N2C(=O)C=C(N3CC(C(=O)O)C3)C2=O)C(=O)N1 ; Cl ; Nc1ncc(-c2cnn(C3CCN(CCC4CCN(C(=O)C5CN(C6=CC(=O)N(C7CCC(=O)NC7=O)C6=O)C5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (20 mg, 0.030 mmol, 1 eq.), 1-(1-(2,6-dioxopiperidin-3-yl)-2,5-dioxo-2,5-dihydro-1H-pyrrol-3-yl)azetidine-3-carboxylic acid (9 mg, 0.030 mmol, 1.0 eq.), EDCI-HCl (6 mg, 0.033 mmol, 1.1 eq.), HOBt (5 mg, 0.033 mmol, 1.1 eq.) and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(1-(1-(2,6-dioxopiperidin-3-yl)-2,5-dioxo-2,5-dihydro-1H-pyrrol-3-yl)azetidine-3-carbonyl) Piperidin-4-yl)ethyl)piperidin-4-yl)-1H-pyrazol-4-yl)pyrazine-2-carboxylate (4 mg, 17%).

### Synthesis of (R)-2-((4-fluorophenyl)amino)-2-oxo-1-phenylethyl 3-amino-6-(1-(1-(2-(1-(4-((2,6-dioxopiperidin-3-yl)carbamoyl)benzoyl)piperidin-4-yl)ethyl)piperidin-4-yl)-1H-pyrazol-4-yl)pyrazine-2-carboxylate [TDH-536]

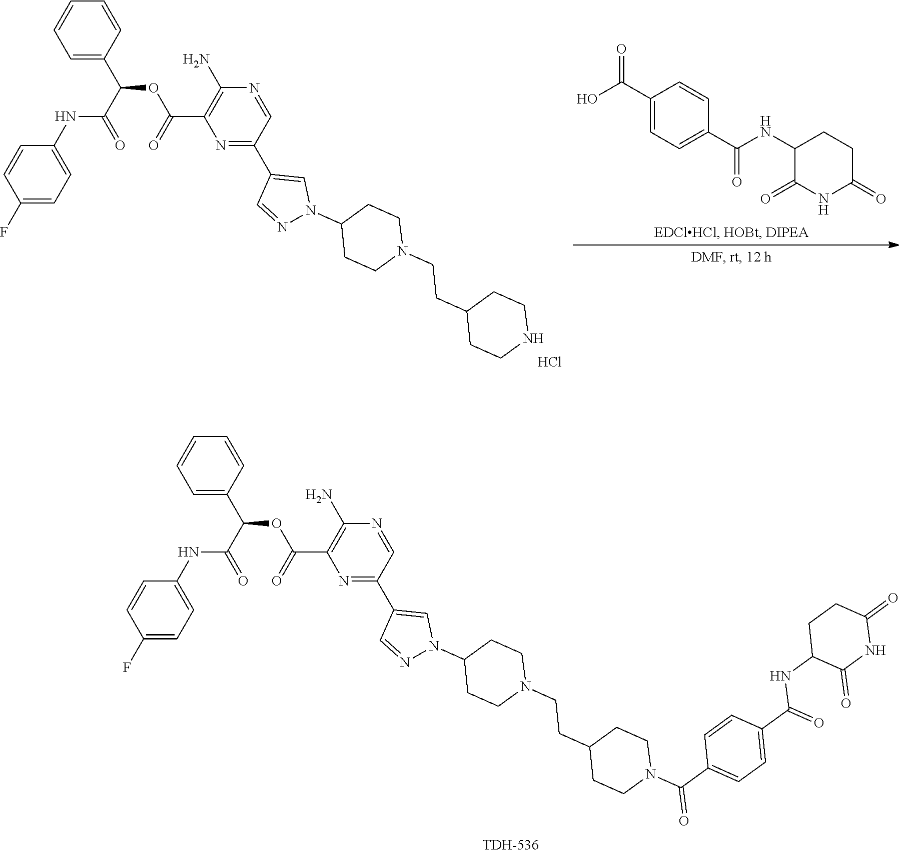 [SMILES: Nc1ncc(-c2cnn(C3CCN(CCC4CCNCC4)CC3)c2)nc1C(=O)O[C@@H](C(=O)Nc1ccc(F)cc1)c1ccccc1 ; O=C1CCC(NC(=O)c2ccc(C(=O)O)cc2)C(=O)N1 ; Cl ; Nc1ncc(-c2cnn(C3CCN(CCC4CCN(C(=O)c5ccc(C(=O)NC6CCC(=O)NC6=O)cc5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (20 mg, 0.030 mmol, 1 eq.), 4-((2,6-dioxopiperidin-3-yl)carbamoyl)benzoic acid (9 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL), and the mixture was reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(4-((2,6-dioxopiperidin-3-yl)carbamoyl)benzoyl)piperidin-4-yl)ethyl)piperidin-4-yl)-1H-pyrazol-4-yl)pyrazine-2-carboxylate (10 mg, 36%).

### Synthesis of (R)-2-((4-fluorophenyl)amino)-2-oxo-1-phenylethyl 3-amino-6-(1-(1-(2-(1-(3-((2,6-dioxopiperidin-3-yl)carbamoyl)benzoyl)piperidin-4-yl)ethyl)piperidin-4-yl)-1H-pyrazol-4-yl)pyrazine-2-carboxylate [TDH-543]

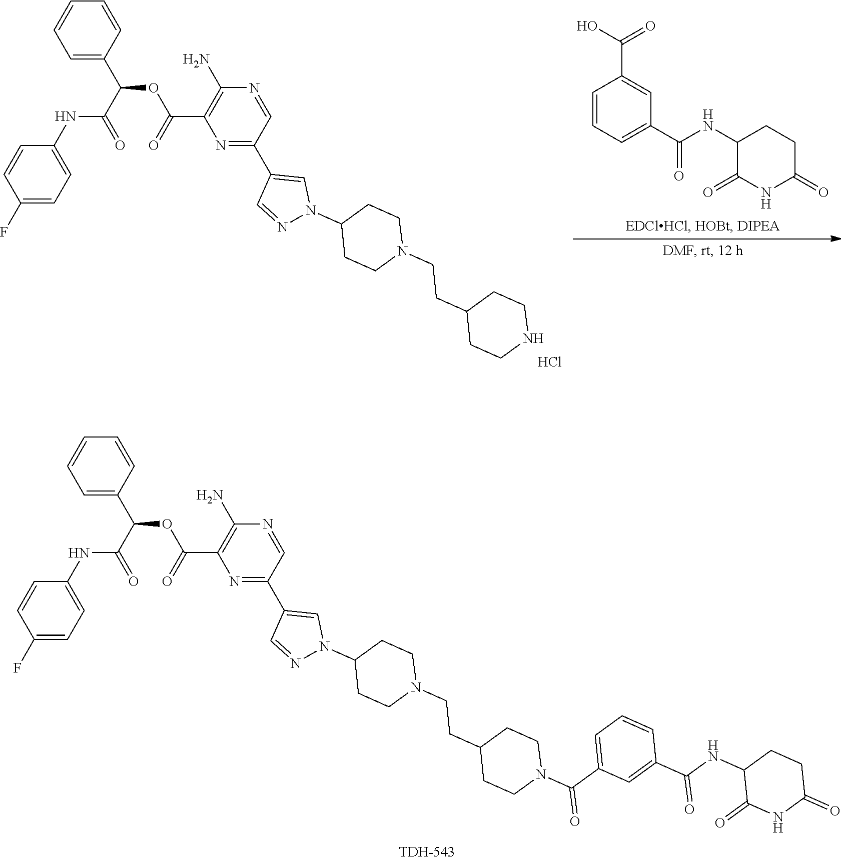 [SMILES: Nc1ncc(-c2cnn(C3CCN(CCC4CCNCC4)CC3)c2)nc1C(=O)O[C@@H](C(=O)Nc1ccc(F)cc1)c1ccccc1 ; O=C1CCC(NC(=O)c2cccc(C(=O)O)c2)C(=O)N1 ; Cl ; Nc1ncc(-c2cnn(C3CCN(CCC4CCN(C(=O)c5cccc(C(=O)NC6CCC(=O)NC6=O)c5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (20 mg, 0.030 mmol, 1 eq.), 3-((2,6-dioxopiperidin-3-yl)carbamoyl)benzoic acid (9 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL), and it was reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(3-((2,6-dioxopiperidin-3-yl)carbamoyl)benzoyl)piperidin-4-yl)ethyl)piperidin-4-yl)-1H-pyrazol-4-yl)pyrazine-2-carboxylate (16 mg, 60%).

### Synthesis of (R)-2-((4-fluorophenyl)amino)-2-oxo-1-phenylethyl 3-amino-6-(1-(1-(2-(1-(2-(2,6-dioxopiperidin-3-yl)-1-oxoisoindoline-5-carbonyl)piperidin-4-yl)ethyl)piperidin-4-yl)-1H-pyrazol-4-yl)pyrazine-2-carboxylate [TDH-547]

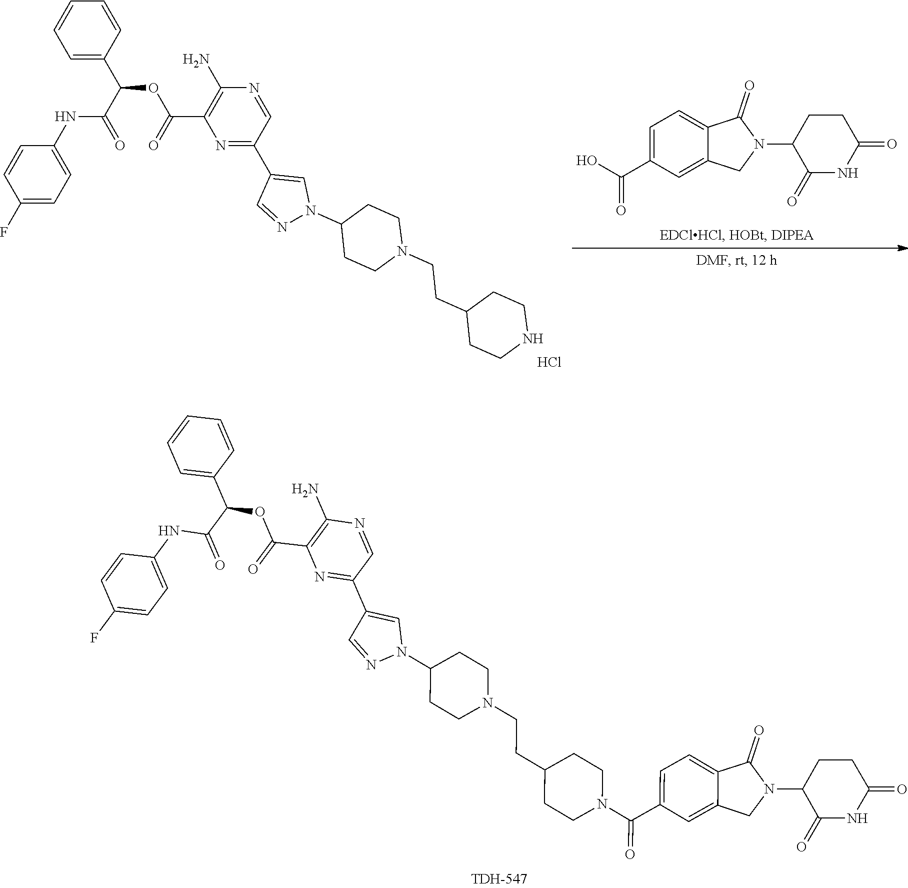 [SMILES: Nc1ncc(-c2cnn(C3CCN(CCC4CCNCC4)CC3)c2)nc1C(=O)O[C@@H](C(=O)Nc1ccc(F)cc1)c1ccccc1 ; O=C1CCC(N2Cc3cc(C(=O)O)ccc3C2=O)C(=O)N1 ; Cl ; Nc1ncc(-c2cnn(C3CCN(CCC4CCN(C(=O)c5ccc6c(c5)CN(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (20 mg, 0.030 mmol, 1 eq.), 2-(2,6-dioxopiperidin-3-yl)-1-oxoisoindoline-5-carboxylic acid (9 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(2-(2,6-dioxopiperidin-3-yl)-1-oxoisoindoline-5-carbonyl)piperidin-4-yl)ethyl)piperidin-4-yl)-1H-pyrazol-4-yl)pyrazine-2-carboxylate (19 mg, 71%).

### Synthesis of (R)-2-((4-fluorophenyl)amino)-2-oxo-1-phenylethyl 3-amino-6-(1-(1-(2-(1-(3-(2,4-dioxotetrahydropyrimidin-1(2H)-yl)benzofuran-6-carbonyl)piperidin-4-yl)ethyl)piperidin-4-yl)-1H-pyrazol-4-yl)pyrazine-2-carboxylate [TDH-552]

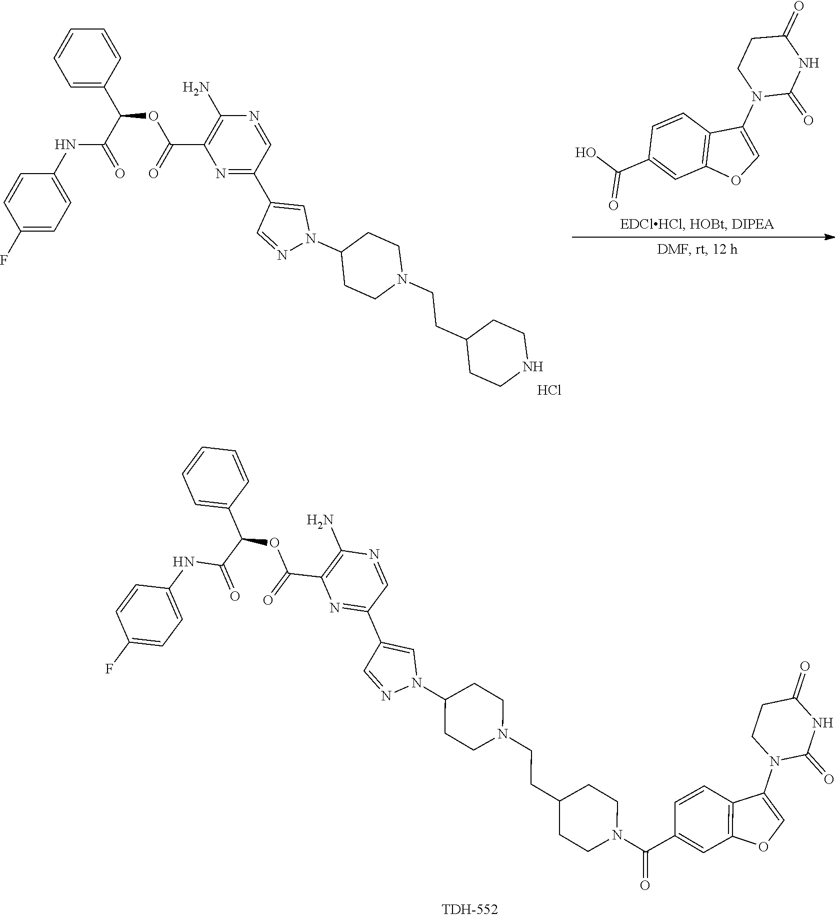 [SMILES: Nc1ncc(-c2cnn(C3CCN(CCC4CCNCC4)CC3)c2)nc1C(=O)O[C@@H](C(=O)Nc1ccc(F)cc1)c1ccccc1 ; O=C1CCN(c2coc3cc(C(=O)O)ccc23)C(=O)N1 ; Cl ; Nc1ncc(-c2cnn(C3CCN(CCC4CCN(C(=O)c5ccc6c(N7CCC(=O)NC7=O)coc6c5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (20 mg, 0.030 mmol, 1 eq.), 3-(2,4-dioxotetrahydropyrimidin-1(2H)-yl)benzofuran-6-carboxylic acid (9 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(3-(2,4-dioxotetrahydropyrimidin-1(2H)-yl)benzofuran-6-carbonyl)piperidin-4-yl)ethyl)piperidin-4-yl)-1H-pyrazol-4-yl)pyrazine-2-carboxylate (17 mg, 64%).

### Synthesis of (R)-2-((4-fluorophenyl)amino)-2-oxo-1-phenylethyl 3-amino-6-(1-(1-(2-(1-(7-(3-(2,4-dioxotetrahydropyrimidin-1(2H)-yl)-4-methoxybenzoyl)-7-azaspiro[3.5]nonan-2-yl)piperidin-4-yl)ethyl)piperidin-4-yl)-1H-pyrazol-4-yl)pyrazine-2-carboxylate [TDH-504]

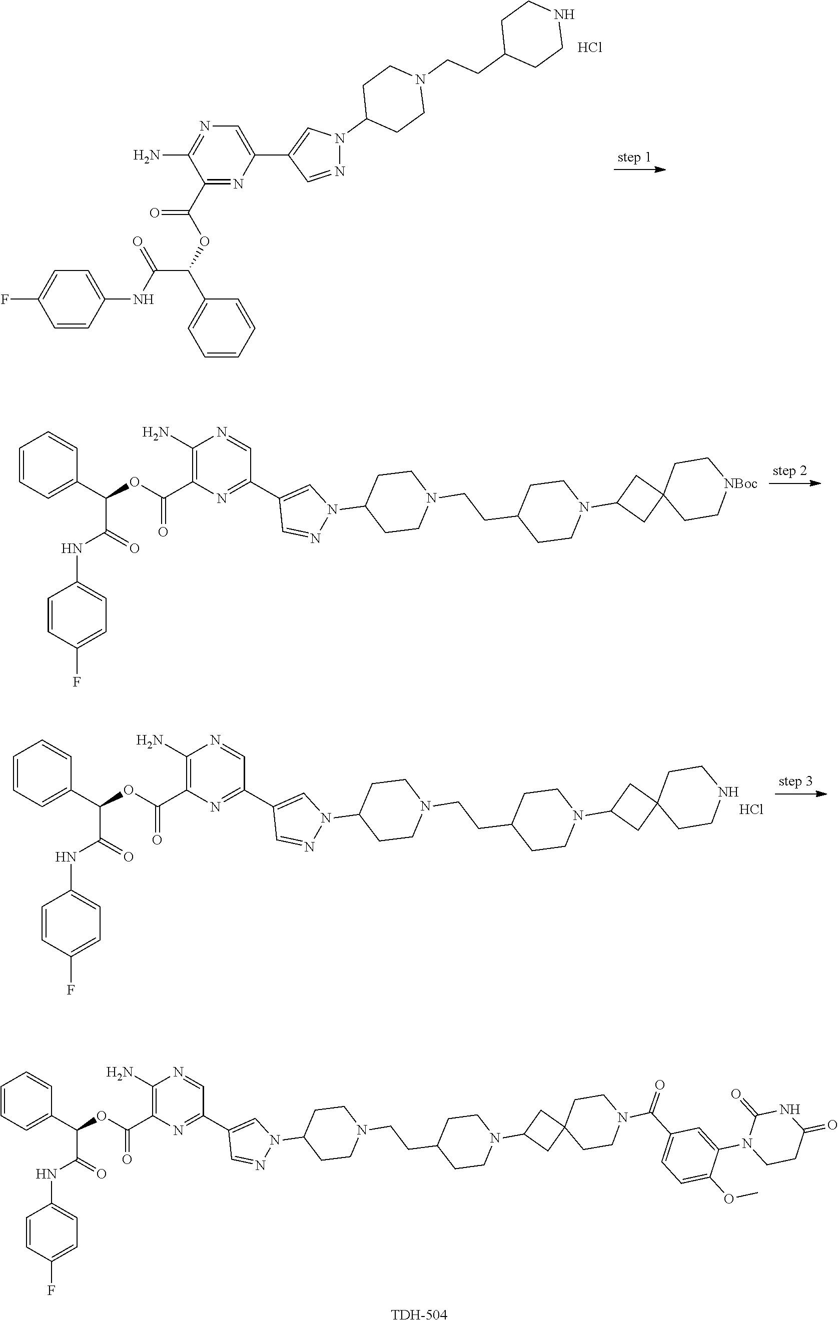 [SMILES: Nc1ncc(-c2cnn(C3CCN(CCC4CCNCC4)CC3)c2)nc1C(=O)O[C@@H](C(=O)Nc1ccc(F)cc1)c1ccccc1 ; Cl ; CC(C)(C)OC(=O)N1CCC2(CC1)CC(N1CCC(CCN3CCC(n4cc(-c5cnc(N)c(C(=O)O[C@@H](C(=O)Nc6ccc(F)cc6)c6ccccc6)n5)cn4)CC3)CC1)C2 ; Nc1ncc(-c2cnn(C3CCN(CCC4CCN(C5CC6(CCNCC6)C5)CC4)CC3)c2)nc1C(=O)O[C@@H](C(=O)Nc1ccc(F)cc1)c1ccccc1 ; Cl ; COc1ccc(C(=O)N2CCC3(CC2)CC(N2CCC(CCN4CCC(n5cc(-c6cnc(N)c(C(=O)O[C@@H](C(=O)Nc7ccc(F)cc7)c7ccccc7)n6)cn5)CC4)CC2)C3)cc1N1CCC(=O)NC1=O]

### Tert-butyl (R)-2-(4-(2-(4-(4-(5-amino-6-((2-((4-fluorophenyl)amino)-2-oxo-1-phenylethoxy)carbonyl)pyrazin-2-yl)-1H-pyrazol-1-yl)piperidin-1-yl)ethyl)piperidin-1-yl)-7-azaspiro[3.5]nonane-7-carboxylate (step 1)

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (200 mg, 0.30 mmol, 1 eq.) was dissolved in DCM (1.2 mL), and DIPEA (0.27 mL, 1.5 mmol, 5.0 eq.) was added at 0° C. After 10 minutes, tert-butyl 2-oxo-7-azaspiro[3.5]nonane-7-carboxylate (108 mg, 0.45 mmol, 1.5 eq.) was added to the above solution and reacted at room temperature for 4 hours. After adding NaBH(OAc)₃ (85 mg, 0.40 mmol, 1.34 eq.) at 0° C., the mixture was reacted overnight at room temperature. After completion of the reaction, the mixture was diluted with water and extracted with DCM. The combined organic layer was filtered after removing the residual water through anhydrous Na₂SO₄. The organic layer was concentrated under reduced pressure. The concentrate was separated and purified by silica gel column chromatography to obtain the desired yellow solid, tert-butyl (R)-2-(4-(2-(4-(4-(5-amino-6-((2-((4-fluorophenyl)amino)-2-oxo-1-phenylethoxy)carbonyl)pyrazin-2-yl)-1H-pyrazol-1-yl)piperidin-1-yl)ethyl)piperidin-1-yl)-7-azaspiro[3.5]nonane-7-carboxylate (62 mg, mixture).

### (R)-2-((4-fluorophenyl)amino)-2-oxo-1-phenylethyl 6-(1-(1-(2-(1-(7-azaspiro[3.5]nonan-2-yl)piperidin-4-yl)ethyl)piperidin-4-yl)-1H-pyrazol-4-yl)-3-aminopyrazine-2-carboxylate hydrochloride (step 2)

Tert-butyl (R)-2-(4-(2-(4-(4-(5-amino-6-((2-((4-fluorophenyl)amino)-2-oxo-1-phenylethoxy)carbonyl)pyrazin-2-yl)-1H-pyrazol-1-yl)piperidin-1-yl)ethyl)piperidin-1-yl)-7-azaspiro[3.5]nonane-7-carboxylate (62 mg, 0.07 mmol) was dissolved in DCM (2.0 mL). Then, 4 N HCl in dioxane (1.0 mL) was slowly added, and the mixture was reacted at room temperature for 1 hour. After completion of the reaction, the precipitate was filtered to obtain (R)-2-((4-fluorophenyl)amino)-2-oxo-1-phenylethyl 6-(1-(1-(2-(1-(7-azaspiro[3.5]nonan-2-yl)piperidin-4-yl)ethyl)piperidin-4-yl)-1H-pyrazol-4-yl)-3-aminopyrazine-2-carboxylate hydrochloride (49 mg, mixture).

### (R)-2-((4-fluorophenyl)amino)-2-oxo-1-phenylethyl 3-amino-6-(1-(1-(2-(1-(7-(3-(2,4-dioxotetrahydropyrimidin-1(2H)-yl)-4-methoxybenzoyl)-7-azaspiro[3.5]nonan-2-yl)piperidin-4-yl)ethyl)piperidin-4-yl)-1H-pyrazol-4-yl)pyrazine-2-carboxylate [step 3, TDH-504]

(R)-2-((4-fluorophenyl)amino)-2-oxo-1-phenylethyl 6-(1-(1-(2-(1-(7-azaspiro[3.5]nonan-2-yl)piperidin-4-yl)ethyl)piperidin-4-yl)-1H-pyrazol-4-yl)-3-aminopyrazine-2-carboxylate hydrochloride (20 mg, 0.025 mmol, 1 eq.), 3-(2,4-dioxotetrahydropyrimidin-1(2H)-yl)-4-methoxybenzoic acid (7 mg, 0.025 mmol, 1.0 eq.), EDCI-HCl (6 mg, 0.028 mmol, 1.1 eq.), HOBt (4 mg, 0.028 mmol, 1.1 eq.) and DIPEA (0.02 mL, 0.13 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(7-(3-(2,4-dioxotetrahydropyrimidin-1(2H)-yl)-4-methoxybenzoyl)-7-azaspiro[3.5]nonan-2-yl)piperidin-4-yl)ethyl)piperidin-4-yl)-1H-pyrazol-4-yl)pyrazine-2-carboxylate (13 mg, 55%).

### Synthesis of (R)-2-((4-fluorophenyl)amino)-2-oxo-1-phenylethyl 3-amino-6-(1-(1-(2-(1-(7-(2-(3-((1-(2,6-dioxopiperidin-3-yl)-2,5-dioxo-2,5-dihydro-1H-pyrrol-3-yl)amino)phenyl)acetyl)-7-azaspiro[3.5]nonan-2-yl)piperidin-4-yl)ethyl)piperidin-4-yl)-1H-pyrazol-4-yl)pyrazine-2-carboxylate [TDH-509]

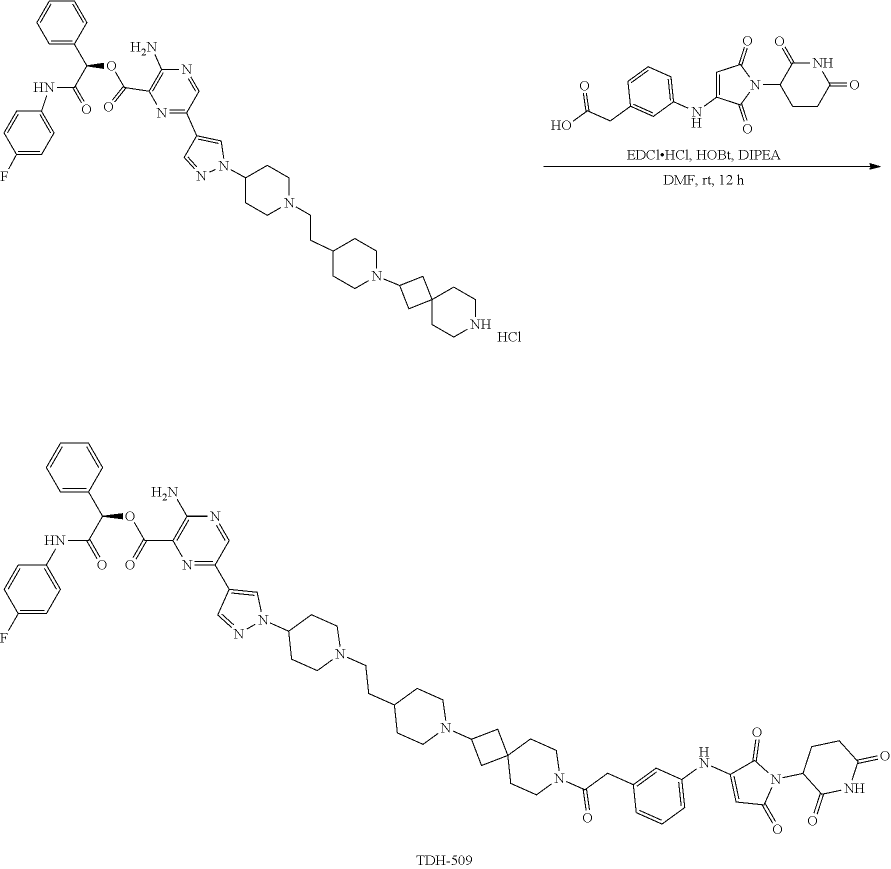 [SMILES: Nc1ncc(-c2cnn(C3CCN(CCC4CCN(C5CC6(CCNCC6)C5)CC4)CC3)c2)nc1C(=O)O[C@@H](C(=O)Nc1ccc(F)cc1)c1ccccc1 ; Cl ; O=C(O)Cc1cccc(NC2=CC(=O)N(C3CCC(=O)NC3=O)C2=O)c1 ; Nc1ncc(-c2cnn(C3CCN(CCC4CCN(C5CC6(CCN(C(=O)Cc7cccc(NC8=CC(=O)N(C9CCC(=O)NC9=O)C8=O)c7)CC6)C5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 6-(1-(1-(2-(1-(7-azaspiro[3.5]nonan-2-yl)piperidin-4-yl)ethyl)piperidin-4-yl)-1H-pyrazol-4-yl)-3-aminopyrazine-2-carboxylate hydrochloride (15 mg, 0.020 mmol, 1 eq.), 2-(3-((1-(2,6-dioxopiperidin-3-yl)-2,5-dioxo-2,5-dihydro-1H-pyrrol-3-yl)amino)phenyl)acetic acid (7 mg, 0.020 mmol, 1.0 eq.), EDCI-HCl (4 mg, 0.022 mmol, 1.1 eq.), HOBt (3 mg, 0.022 mmol, 1.1 eq.), and DIPEA (0.017 mL, 0.10 mmol, 5 eq.) were dissolved in DMF (1.0 mL), and it was reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(7-(2-(3-((1-(2,6-dioxopiperidin-3-yl)-2,5-dioxo-2,5-dihydro-1H-pyrrol-3-yl)amino)phenyl)acetyl)-7-azaspiro[3.5]nonan-2-yl)piperidin-4-yl)ethyl)piperidin-4-yl)-1H-pyrazol-4-yl)pyrazine-2-carboxylate (12 mg, 42%).

### Synthesis of (R)-2-((4-fluorophenyl)amino)-2-oxo-1-phenylethyl 3-amino-6-(1-(1-(2-(1-((1-(3-(2,4-dioxotetrahydropyrimidin-1(2H)-yl)-4-methoxybenzoyl)piperidin-4-yl)methyl)piperidin-4-yl)ethyl)piperidin-4-yl)-1H-pyrazol-4-yl)pyrazine-2-carboxylate [TDH-505]

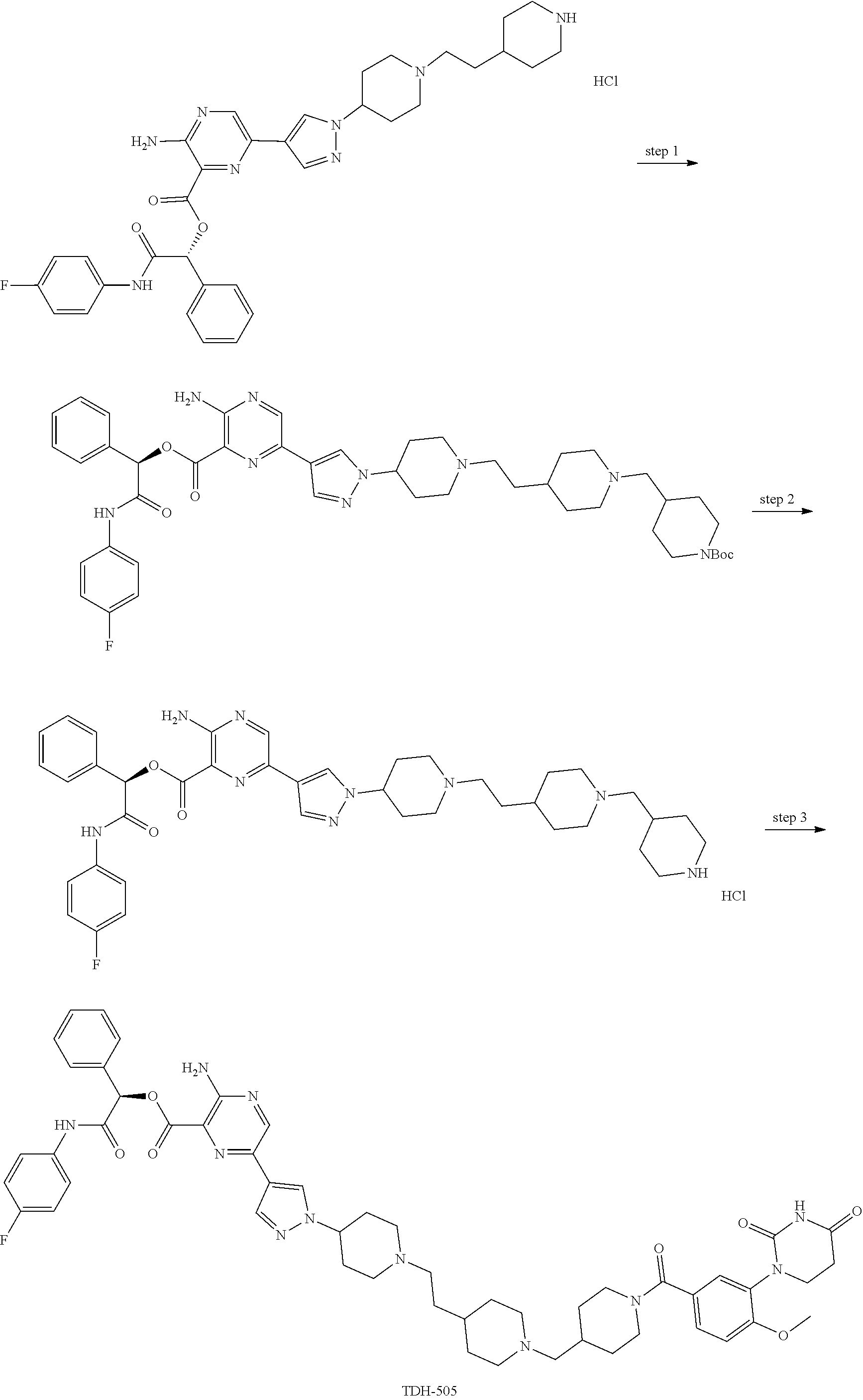

### (R)-2-((4-fluorophenyl)amino)-2-oxo-1-phenylethyl 3-amino-6-(1-(1-(2-(1-((1-(tert-butoxycarbonyl)piperidin-4-yl)methyl)piperidin-4-yl)ethyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(1-(2-(piperidin-4-yl)ethyl)piperidin-4-yl)-1H-pyrazol-4-yl)pyrazine-2-carboxylate hydrochloride (200 mg, 0.30 mmol, 1 eq.) was dissolved in DCM (1.2 mL), and DIPEA (0.27 mL, 1.5 mmol, 5.0 eq.) was added at 0° C. After 10 minutes, tert-butyl 4-formylpiperidine-1-carboxylate (96 mg, 0.45 mmol, 1.5 eq.) was added to the above solution and reacted at room temperature for 4 hours. After adding NaBH(OAc)₃ (85 mg, 0.40 mmol, 1.34 eq.) at 0° C., the mixture was reacted overnight at room temperature. After completion of the reaction, the mixture was diluted with water and extracted with DCM. The combined organic layer was filtered after removing the residual water through anhydrous Na₂SO₄. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-((1-(tert-butoxycarbonyl)piperidin-4-yl)methyl)piperidin-4-yl)ethyl)piperidin-4-yl)-1H-pyrazol-4-yl)pyrazine-2-carboxylate (229 mg, mixture).

### (R)-2-((4-fluorophenyl)amino)-2-oxo-1-phenylethyl 3-amino-6-(1-(1-(2-(1-(piperidin-4-ylmethyl)piperidin-4-yl)ethyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-(2-(1-((1-(tert-butoxycarbonyl)piperidin-4-yl)methyl)piperidin-4-yl)ethyl)piperidin-4-yl)-1H-pyrazol-4-yl)pyrazine-2-carboxylate (229 mg, 0.28 mmol) was dissolved in DCM (5.0 mL). Then, 4 N HCl in dioxane (2.0 mL) was slowly added and reacted at room temperature for 1 hour. After completion of the reaction, the precipitate was filtered to obtain (R)-2-((4-fluorophenyl)amino)-2-oxo-1-phenylethyl 3-amino-6-(1-(1-(2-(1-(piperidin-4-ylmethyl)piperidin-4-yl)ethyl)piperidin-4-yl)-1H-pyrazol-4-yl)pyrazine-2-carboxylate hydrochloride (182 mg, mixture).

### (R)-2-((4-fluorophenyl)amino)-2-oxo-1-phenylethyl 3-amino-6-(1-(1-(2-(1-((1-(3-(2,4-dioxotetrahydropyrimidin-1(2H)-yl)-4-methoxybenzoyl)piperidin-4-yl)methyl)piperidin-4-yl)ethyl)piperidin-4-yl)-1H-pyrazol-4-yl)pyrazine-2-carboxylate [step 3, TDH-505]

(R)-2-((4-fluorophenyl)amino)-2-oxo-1-phenylethyl 3-amino-6-(1-(1-(2-(1-(piperidin-4-ylmethyl)piperidin-4-yl)ethyl)piperidin-4-yl)-1H-pyrazol-4-yl)pyrazine-2-carboxylate hydrochloride (20 mg, 0.026 mmol, 1 eq.), 3-(2,4-dioxotetrahydropyrimidin-1(2H)-yl)-4-methoxybenzoic acid (7 mg, 0.026 mmol, 1.0 eq.), EDCI-HCl (6 mg, 0.029 mmol, 1.1 eq.), HOBt (4 mg, 0.029 mmol, 1.1 eq.), and DIPEA (0.02 mL, 0.13 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-((1-(3-(2,4-dioxotetrahydropyrimidin-1(2H)-yl)-4-methoxybenzoyl)piperidin-4-yl)methyl)piperidin-4-yl)ethyl)piperidin-4-yl)-1H-pyrazol-4-yl)pyrazine-2-carboxylate (17 mg, 70%).

### Synthesis of (R)-2-((4-fluorophenyl)amino)-2-oxo-1-phenylethyl 3-amino-6-(1-(1-(2-(1-((1-(2-(3-((1-(2,6-dioxopiperidin-3-yl)-2,5-dioxo-2,5-dihydro-1H-pyrrol-3-yl)amino)phenyl)acetyl)piperidin-4-yl)methyl)piperidin-4-yl)ethyl)piperidin-4-yl)-1H-pyrazol-4-yl)pyrazine-2-carboxylate [TDH-510]

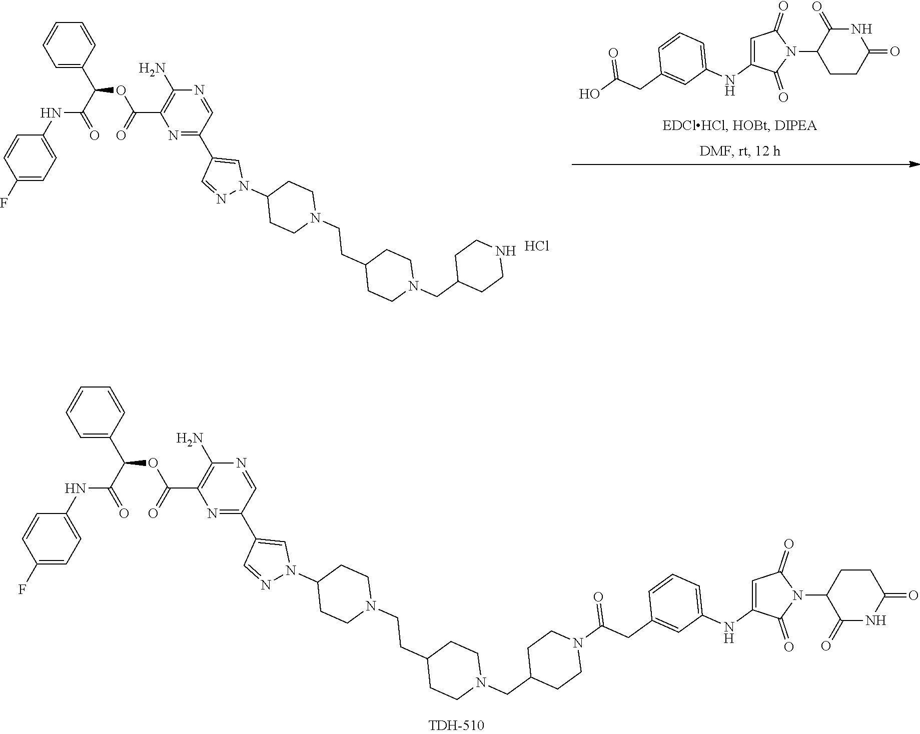 [SMILES: Nc1ncc(-c2cnn(C3CCN(CCC4CCN(CC5CCNCC5)CC4)CC3)c2)nc1C(=O)O[C@@H](C(=O)Nc1ccc(F)cc1)c1ccccc1 ; Cl ; O=C(O)Cc1cccc(NC2=CC(=O)N(C3CCC(=O)NC3=O)C2=O)c1 ; Nc1ncc(-c2cnn(C3CCN(CCC4CCN(CC5CCN(C(=O)Cc6cccc(NC7=CC(=O)N(C8CCC(=O)NC8=O)C7=O)c6)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(1-(piperidin-4-ylmethyl)piperidin-4-yl)ethyl)piperidin-4-yl)-1H-pyrazol-4-yl)pyrazine-2-carboxylate hydrochloride (20 mg, 0.026 mmol, 1 eq.), 2-(3-((1-(2,6-dioxopiperidin-3-yl)-2,5-dioxo-2,5-dihydro-1H-pyrrol-3-yl)amino)phenyl)acetic acid (10 mg, 0.026 mmol, 1.0 eq.), EDCI-HCl (6 mg, 0.029 mmol, 1.1 eq.), HOBt (4 mg, 0.029 mmol, 1.1 eq.), and DIPEA (0.02 mL, 0.13 mmol, 5 eq.) were dissolved in DMF (1.0 mL) and reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-((1-(2-(3-((1-(2,6-dioxopiperidin-3-yl)-2,5-dioxo-2,5-dihydro-1H-pyrrol-3-yl)amino)phenyl)acetyl)piperidin-4-yl)methyl)piperidin-4-yl)ethyl)piperidin-4-yl)-1H-pyrazol-4-yl)pyrazine-2-carboxylate (20 mg, 72%).

### Synthesis of (R)-2-((4-fluorophenyl)amino)-2-oxo-1-phenylethyl 3-amino-6-(1-(1-(2-(1-((1-(1-(3-(2,4-dioxotetrahydropyrimidin-1(2H)-yl)-4-methoxybenzoyl)piperidine-4-carbonyl)piperidin-4-yl)methyl)piperidin-4-yl)ethyl)piperidin-4-yl)-1H-pyrazol-4-yl)pyrazine-2-carboxylate [TDH-511]

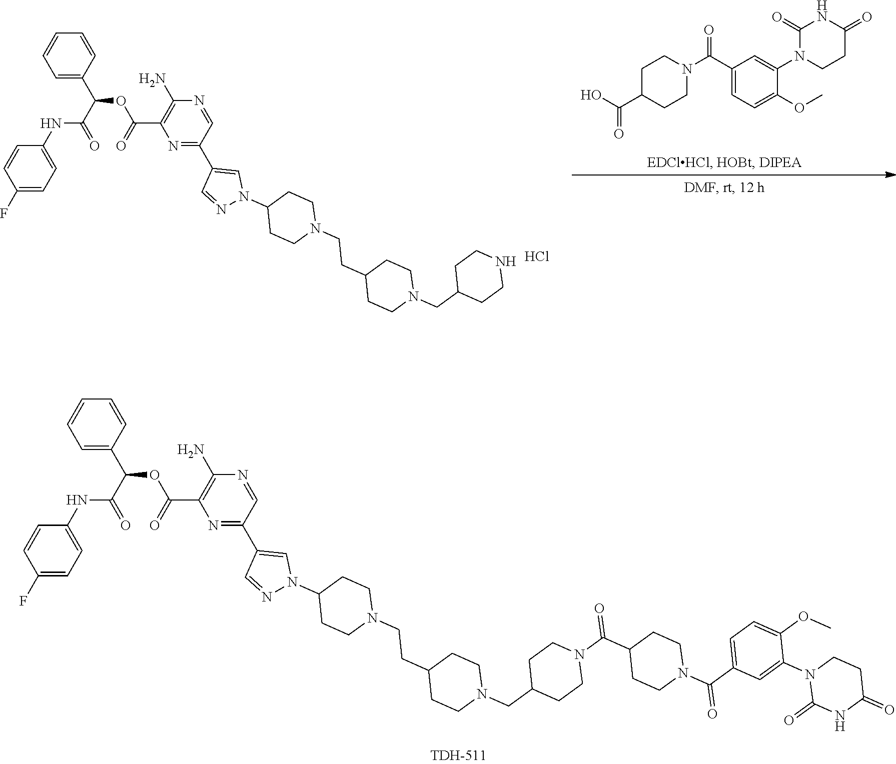 [SMILES: Nc1ncc(-c2cnn(C3CCN(CCC4CCN(CC5CCNCC5)CC4)CC3)c2)nc1C(=O)O[C@@H](C(=O)Nc1ccc(F)cc1)c1ccccc1 ; Cl ; COc1ccc(C(=O)N2CCC(C(=O)O)CC2)cc1N1CCC(=O)NC1=O ; COc1ccc(C(=O)N2CCC(C(=O)N3CCC(CN4CCC(CCN5CCC(n6cc(-c7cnc(N)c(C(=O)O[C@@H](C(=O)Nc8ccc(F)cc8)c8ccccc8)n7)cn6)CC5)CC4)CC3)CC2)cc1N1CCC(=O)NC1=O]

(R)-2-((4-fluorophenyl)amino)-2-oxo-1-phenylethyl 3-amino-6-(1-(1-(2-(1-(piperidin-4-ylmethyl)piperidin-4-yl)ethyl)piperidin-4-yl)-1H-pyrazol-4-yl)pyrazine-2-carboxylate hydrochloride (20 mg, 0.026 mmol, 1 eq.), 1-(3-(2,4-dioxotetrahydropyrimidin-1(2H)-yl)-4-methoxybenzoyl)piperidine-4-carboxylic acid (10 mg, 0.026 mmol, 1.0 eq.), EDCI-HCl (6 mg, 0.029 mmol, 1.1 eq.), HOBt (4 mg, After dissolving 0.029 mmol, 1.1 eq.) and DIPEA (0.02 mL, 0.13 mmol, 5 eq.) were dissolved in DMF (1.0 mL), and the mixture was reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-((1-(1-(3-(2,4-dioxotetrahydropyrimidin-1(2H)-yl)-4-methoxybenzoyl)piperidine-4-carbonyl)piperidin-4-yl)methyl)piperidin-4-yl)ethyl)piperidin-4-yl)-1H-pyrazol-4-yl)pyrazine-2-carboxylate (10 mg, 36%).

### Synthesis of (R)-2-((4-fluorophenyl)amino)-2-oxo-1-phenylethyl 3-amino-6-(1-(1-(2-(1-((1-(2-(2,6-dioxopiperidin-3-yl)-6-fluoro-1,3-dioxoisoindolin-5-yl)piperidin-4-yl)methyl)piperidin-4-yl)ethyl)piperidin-4-yl)-1H-pyrazol-4-yl)pyrazine-2-carboxylate [TDH-514]

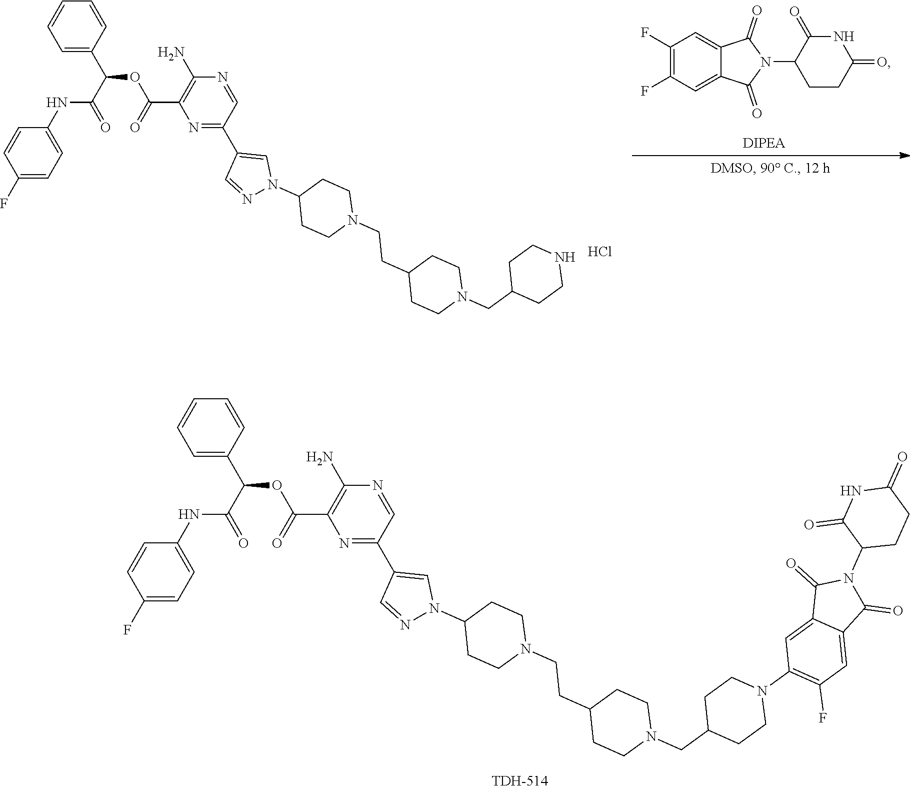 [SMILES: Nc1ncc(-c2cnn(C3CCN(CCC4CCN(CC5CCNCC5)CC4)CC3)c2)nc1C(=O)O[C@@H](C(=O)Nc1ccc(F)cc1)c1ccccc1 ; Cl ; O=C1CCC(N2C(=O)c3cc(F)c(F)cc3C2=O)C(=O)N1 ; Nc1ncc(-c2cnn(C3CCN(CCC4CCN(CC5CCN(c6cc7c(cc6F)C(=O)N(C6CCC(=O)NC6=O)C7=O)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(1-(piperidin-4-ylmethyl)piperidin-4-yl)ethyl)piperidin-4-yl)-1H-pyrazol-4-yl)pyrazine-2-carboxylate hydrochloride (20 mg, 0.026 mmol, 1 eq.), 2-(2,6-dioxopiperidin-3-yl))-5,6-difluoroisoindoline-1,3-dione (7 mg, 0.025 mmol, 0.95 eq.) and DIPEA (0.014 mL, 0.079 mmol, 3 eq.) were dissolved in DMSO (1.0 mL) and reacted at 90° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(2-(2,6-dioxopiperidin-3-yl)-6-fluoro-1,3-dioxoisoindolin-5-yl)piperidin-4-yl)ethyl)piperidin-4-yl)-1H-pyrazol-4-yl)pyrazine-2-carboxylate (15 mg, 58%).

### Synthesis of (R)-2-((4-fluorophenyl)amino)-2-oxo-1-phenylethyl 3-amino-6-(1-(1-(3-(1-(2-(2,6-dioxopiperidin-3-yl)-1,3-dioxoisoindolin-5-yl)piperidin-4-yl)propyl)piperidin-4-yl)-1H-pyrazol-4-yl)pyrazine-2-carboxylate [TDH-481]

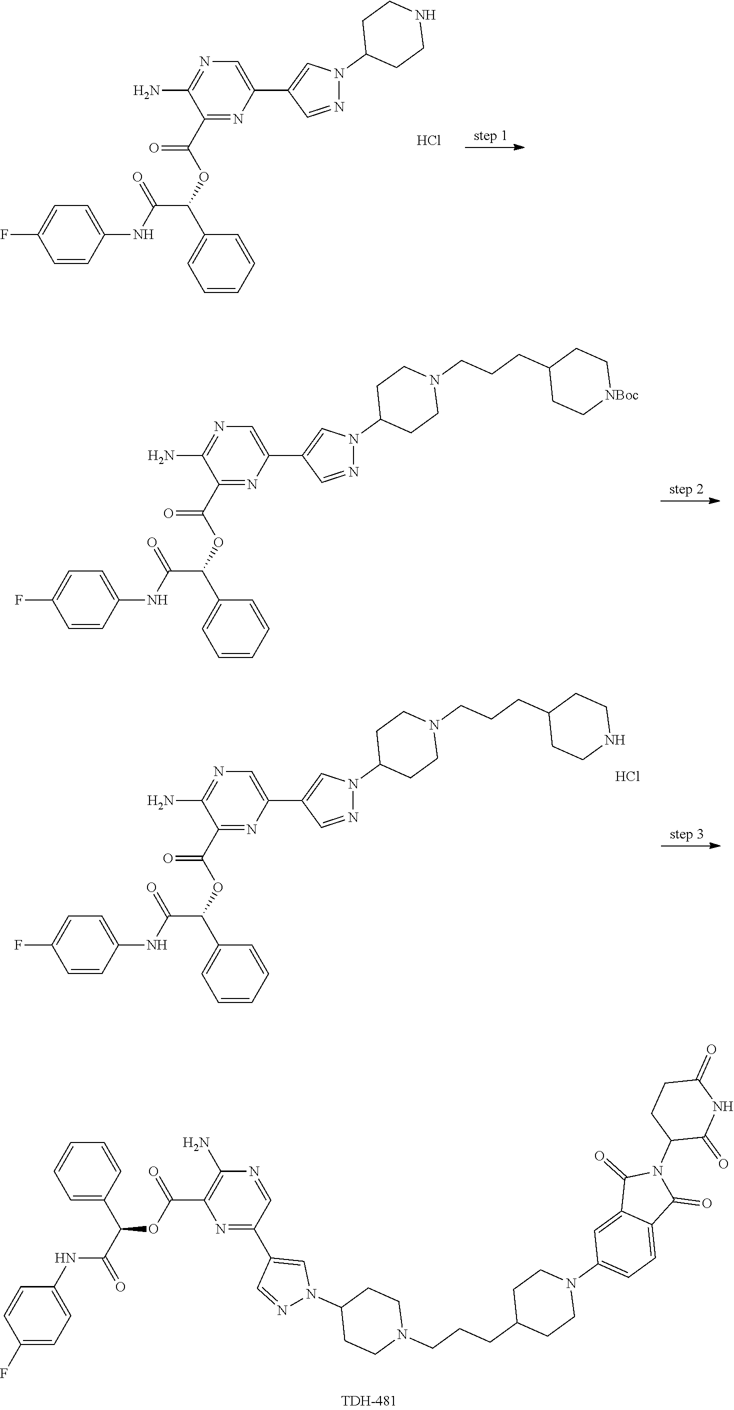 [SMILES: Nc1ncc(-c2cnn(C3CCNCC3)c2)nc1C(=O)O[C@@H](C(=O)Nc1ccc(F)cc1)c1ccccc1 ; Cl ; CC(C)(C)OC(=O)N1CCC(CCCN2CCC(n3cc(-c4cnc(N)c(C(=O)O[C@@H](C(=O)Nc5ccc(F)cc5)c5ccccc5)n4)cn3)CC2)CC1 ; Nc1ncc(-c2cnn(C3CCN(CCCC4CCNCC4)CC3)c2)nc1C(=O)O[C@@H](C(=O)Nc1ccc(F)cc1)c1ccccc1 ; Cl ; Nc1ncc(-c2cnn(C3CCN(CCCC4CCN(c5ccc6c(c5)C(=O)N(C5CCC(=O)NC5=O)C6=O)CC4)CC3)c2)nc1C(=O)O[C@@H](C(=O)Nc1ccc(F)cc1)c1ccccc1]

### (R)-2-((4-fluorophenyl)amino)-2-oxo-1-phenylethyl 3-amino-6-(1-(1-(3-(1-(tert-butoxycarbonyl)piperidin-4-yl)propyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylate hydrochloride (20 mg, 0.036 mmol, 1.0 eq.), tert-butyl 4-(3-iodopropyl)piperidine-1-carboxylate (19 mg, 0.054 mmol, 1.5 eq.), and K₂CO₃ (10 mg, 0.072 mmol, 2.0 eq.) were dissolved in DMF (1 mL) and reacted overnight at 50° C. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(1-(tert-butoxycarbonyl)piperidin-4-yl)propyl)piperidin-4-yl)-1H-pyrazol-4-yl)pyrazine-2-carboxylate (20 mg, 78%).

¹H NMR (500 MHz, CDCl₃) δ 9.03 (s, 1H), 8.53 (s, 1H), 7.99 (s, 1H), 7.95 (s, 1H), 7.69-7.63 (m, 2H), 7.61-7.55 (m, 2H), 7.51-7.37 (m, 3H), 7.09-7.01 (m, 2H), 6.45 (s, 1H), 6.36 (s, 2H), 4.31-4.22 (m, 1H), 4.19-3.98 (m, 2H), 3.16-3.07 (m, 2H), 2.75-2.64 (m, 2H), 2.46-2.39 (m, 2H), 2.28-2.16 (m, 4H), 2.16-2.03 (m, 3H), 1.73-1.66 (m, 2H), 1.63-1.54 (m, 3H), 1.48 (s, 9H), 1.45-1.36 (m, 2H), 1.18-1.05 (m, 3H).

### (R)-2-((4-fluorophenyl)amino)-2-oxo-1-phenylethyl 3-amino-6-(1-(1-(3-(piperidin-4-yl)propyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-(3-(1-(tert-butoxycarbonyl)piperidin-4-yl)propyl)piperidin-4-yl)-1H-pyrazol-4-yl)pyrazine-2-carboxylate (140 mg, 0.045 mmol) was dissolved in DCM (2.0 mL). Then, 4 N HCl in dioxane (1.0 mL) was slowly added, and reacted at room temperature for 1 hour. After completion of the reaction, the precipitate was filtered to obtain (R)-2-((4-fluorophenyl)amino)-2-oxo-1-phenylethyl 3-amino-6-(1-(1-(3-(piperidin-4-yl)propyl)piperidin-4-yl)-1H-pyrazol-4-yl)pyrazine-2-carboxylate hydrochloride (114 mg, 89%).

¹H NMR (400 MHz, MeOD) δ 8.64-8.58 (m, 1H), 8.34-8.30 (m, 1H), 8.09-8.04 (m, 1H), 7.77-7.72 (m, 2H), 7.62-7.56 (m, 2H), 7.53-7.45 (m, 3H), 7.11-7.04 (m, 2H), 6.37 (s, 1H), 4.69-4.61 (m, 1H), 3.82-3.76 (m, 2H), 3.44-3.40 (m, 2H), 3.25-3.20 (m, 2H), 3.05-2.99 (m, 3H), 2.49-2.39 (m, 4H), 2.04-1.99 (m, 2H), 1.92-1.84 (m, 2H), 1.77-1.68 (m, 1H), 1.52-1.37 (m, 6H).

### (R)-2-((4-fluorophenyl)amino)-2-oxo-1-phenylethyl 3-amino-6-(1-(1-(3-(1-(2-(2,6-dioxopiperidin-3-yl)-1,3-dioxoisoindolin-5-yl)piperidin-4-yl)propyl)piperidin-4-yl)-1H-pyrazol-4-yl)pyrazine-2-carboxylate [step 3, TDH-481]

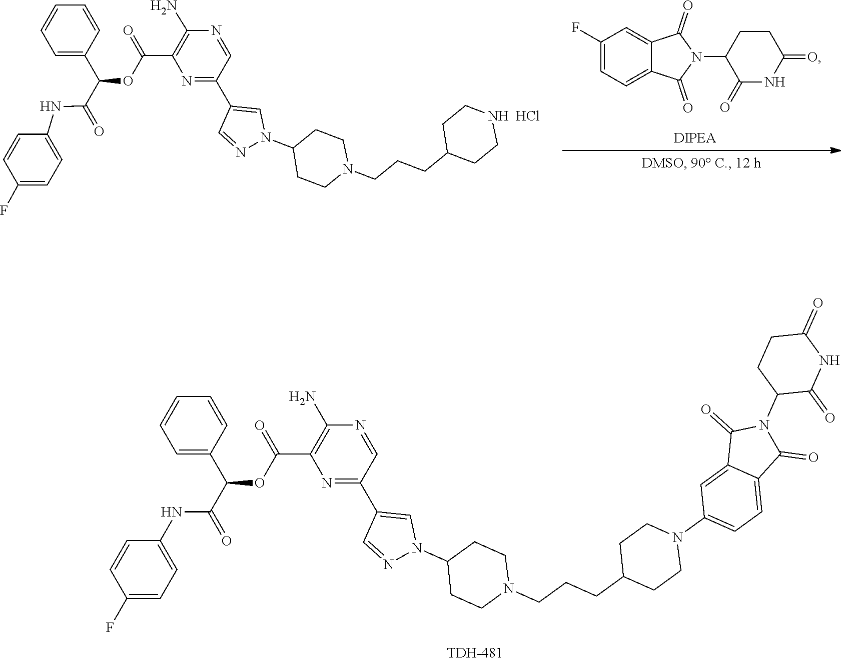 [SMILES: Nc1ncc(-c2cnn(C3CCN(CCCC4CCNCC4)CC3)c2)nc1C(=O)O[C@@H](C(=O)Nc1ccc(F)cc1)c1ccccc1 ; Cl ; O=C1CCC(N2C(=O)c3ccc(F)cc3C2=O)C(=O)N1 ; Nc1ncc(-c2cnn(C3CCN(CCCC4CCN(c5ccc6c(c5)C(=O)N(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperidin-4-yl)propyl)piperidin-4-yl)-1H-pyrazol-4-yl)pyrazine-2-carboxylate hydrochloride (20 mg, 0.030 mmol, 1 eq.), 2-(2,6-dioxopiperidin-3-yl)-5-fluoroisoindoline-1,3-dione (8 mg, 0.030 mmol, 1 eq.) and DIPEA (0.015 mL, 0.089 mmol, 3 eq.) were dissolved in DMSO (1.0 mL), and the mixture was reacted at 90° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(1-(2-(2,6-dioxopiperidin-3-yl)-1,3-dioxoisoindolin-5-yl)piperidin-4-yl)propyl)piperidin-4-yl)-1H-pyrazol-4-yl)pyrazine-2-carboxylate (16 mg, 61%).

### Synthesis of (R)-2-((4-fluorophenyl)amino)-2-oxo-1-phenylethyl 3-amino-6-(1-(1-(3-(1-(2-((2-(2,6-dioxopiperidin-3-yl)-1,3-dioxoisoindolin-5-yl)oxy)acetyl)piperidin-4-yl)propyl)piperidin-4-yl)-1H-pyrazol-4-yl)pyrazine-2-carboxylate [TDH-482]

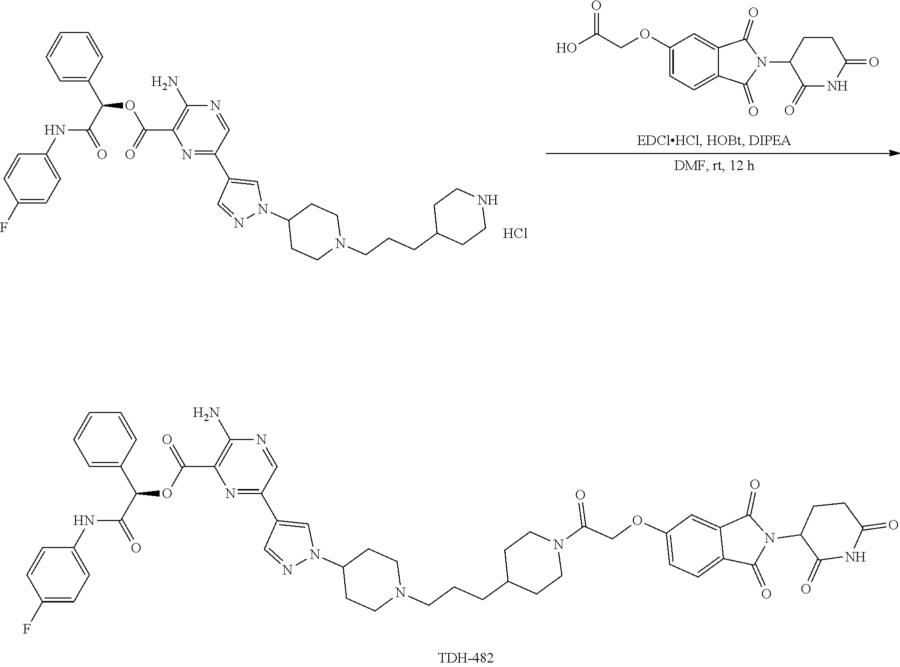 [SMILES: Nc1ncc(-c2cnn(C3CCN(CCCC4CCNCC4)CC3)c2)nc1C(=O)O[C@@H](C(=O)Nc1ccc(F)cc1)c1ccccc1 ; Cl ; O=C(O)COc1ccc2c(c1)C(=O)N(C1CCC(=O)NC1=O)C2=O ; Nc1ncc(-c2cnn(C3CCN(CCCC4CCN(C(=O)COc5ccc6c(c5)C(=O)N(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperidin-4-yl)propyl)piperidin-4-yl)-1H-pyrazol-4-yl)pyrazine-2-carboxylate hydrochloride (20 mg, 0.030 mmol, 1 eq.), 2-((2-(2,6-dioxopiperidin-3-yl)-1,3-dioxoisoindolin-5-yl)oxy)acetic acid (10 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL), and the mixture was reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(1-(2-((2-(2,6-dioxopiperidin-3-yl)-1,3-dioxoisoindolin-5-yl)oxy)acetyl)piperidin-4-yl)propyl)piperidin-4-yl)-1H-pyrazol-4-yl)pyrazine-2-carboxylate (15 mg, 52%).

### Synthesis of (R)-2-((4-fluorophenyl)amino)-2-oxo-1-phenylethyl 3-amino-6-(1-(1-(3-(1-((2-(2,6-dioxopiperidin-3-yl)-1,3-dioxoisoindolin-5-yl)glycyl)piperidin-4-yl)propyl)piperidin-4-yl)-1H-pyrazol-4-yl)pyrazine-2-carboxylate [TDH-483]

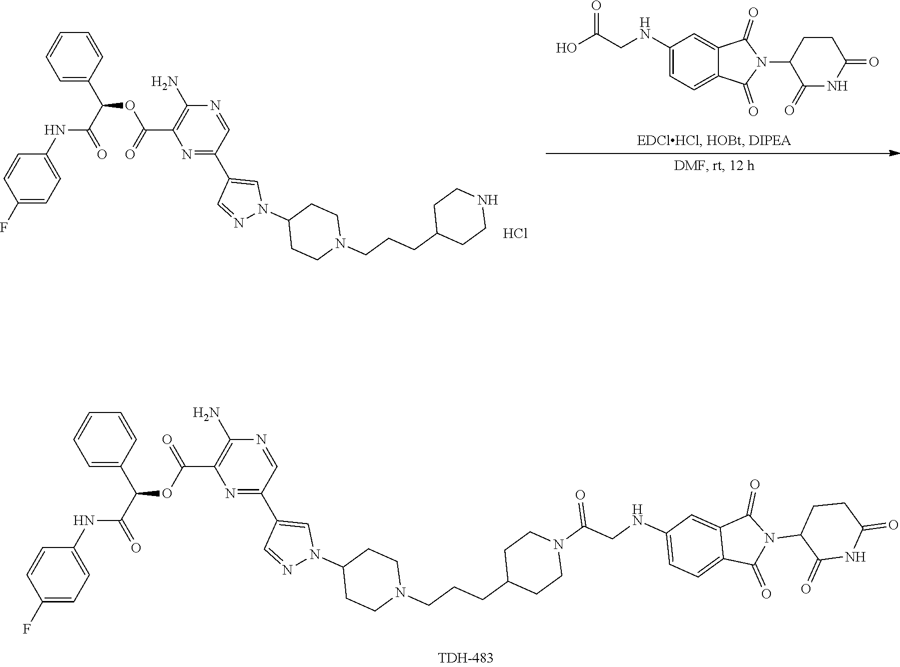 [SMILES: Nc1ncc(-c2cnn(C3CCN(CCCC4CCNCC4)CC3)c2)nc1C(=O)O[C@@H](C(=O)Nc1ccc(F)cc1)c1ccccc1 ; Cl ; O=C(O)CNc1ccc2c(c1)C(=O)N(C1CCC(=O)NC1=O)C2=O ; Nc1ncc(-c2cnn(C3CCN(CCCC4CCN(C(=O)CNc5ccc6c(c5)C(=O)N(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperidin-4-yl)propyl)piperidin-4-yl)-1H-pyrazol-4-yl)pyrazine-2-carboxylate hydrochloride (20 mg, 0.030 mmol, 1 eq.), (2-(2,6-dioxopiperidin-3-yl)-1,3-dioxoisoindolin-5-yl)glycine (10 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL) and reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(1-((2-(2,6-dioxopiperidin-3-yl)-1,3-dioxoisoindolin-5-yl)glycyl)piperidin-4-yl)propyl)piperidin-4-yl)-1H-pyrazol-4-yl)pyrazine-2-carboxylate (11 mg, 38%).

### Synthesis of (R)-2-((4-fluorophenyl)amino)-2-oxo-1-phenylethyl 3-amino-6-(1-(1-(3-(1-(3-(2-(2,6-dioxopiperidin-3-yl)-1,3-dioxoisoindolin-5-yl)propanoyl)piperidin-4-yl)propyl)piperidin-4-yl)-1H-pyrazol-4-yl)pyrazine-2-carboxylate [TDH-484]

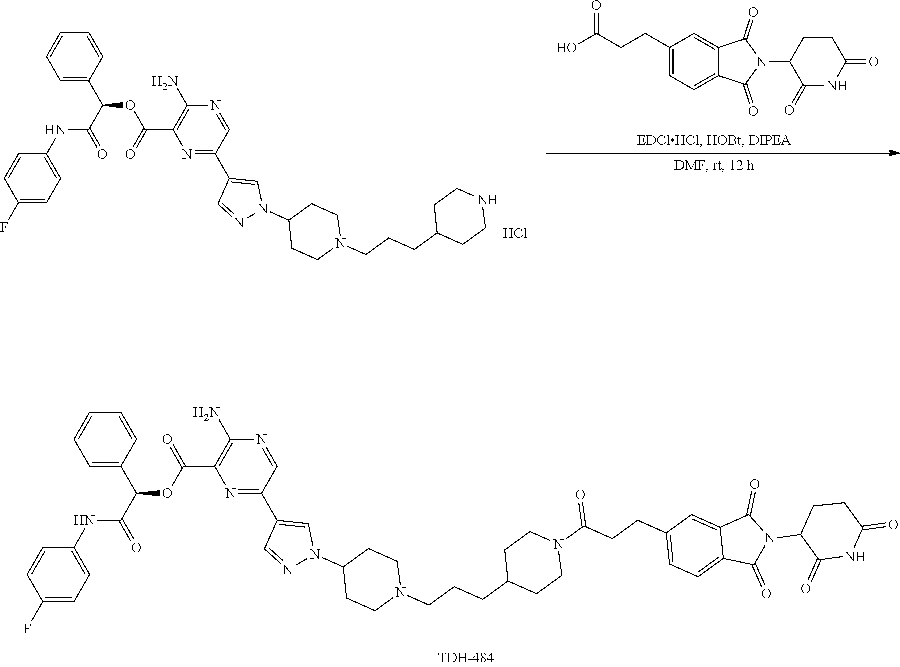 [SMILES: Nc1ncc(-c2cnn(C3CCN(CCCC4CCNCC4)CC3)c2)nc1C(=O)O[C@@H](C(=O)Nc1ccc(F)cc1)c1ccccc1 ; Cl ; O=C(O)CCc1ccc2c(c1)C(=O)N(C1CCC(=O)NC1=O)C2=O ; Nc1ncc(-c2cnn(C3CCN(CCCC4CCN(C(=O)CCc5ccc6c(c5)C(=O)N(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(piperidin-4-yl)propyl)piperidin-4-yl)-1H-pyrazol-4-yl)pyrazine-2-carboxylate hydrochloride (20 mg, 0.030 mmol, 1 eq.), 3-(2-(2,6-dioxopiperidin-3-yl)-1,3-dioxoisoindolin-5-yl)propanoic acid (10 mg, 0.030 mmol, 1.0 eq.), EDCI-HCl (6 mg, 0.033 mmol, 1.1 eq.), HOBt (5 mg, 0.033 mmol, 1.1 eq.), and DIPEA (0.03 mL, 0.15 mmol, 5 eq.) were dissolved in DMF (1.0 mL) and reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(1-(3-(2-(2,6-dioxopiperidin-3-yl)-1,3-dioxoisoindolin-5-yl)propanoyl)piperidin-4-yl)propyl)piperidin-4-yl)-1H-pyrazol-4-yl)pyrazine-2-carboxylate (12 mg, 42%).

### Synthesis of (R)-2-((4-fluorophenyl)amino)-2-oxo-1-phenylethyl 3-amino-6-(1-(1-(2-((1-(3-(2,4-dioxotetrahydropyrimidin-1(2H)-yl)-4-methoxybenzoyl)piperidin-4-yl)oxy)ethyl)piperidin-4-yl)-1H-pyrazol-4-yl)pyrazine-2-carboxylate [TDH-448]

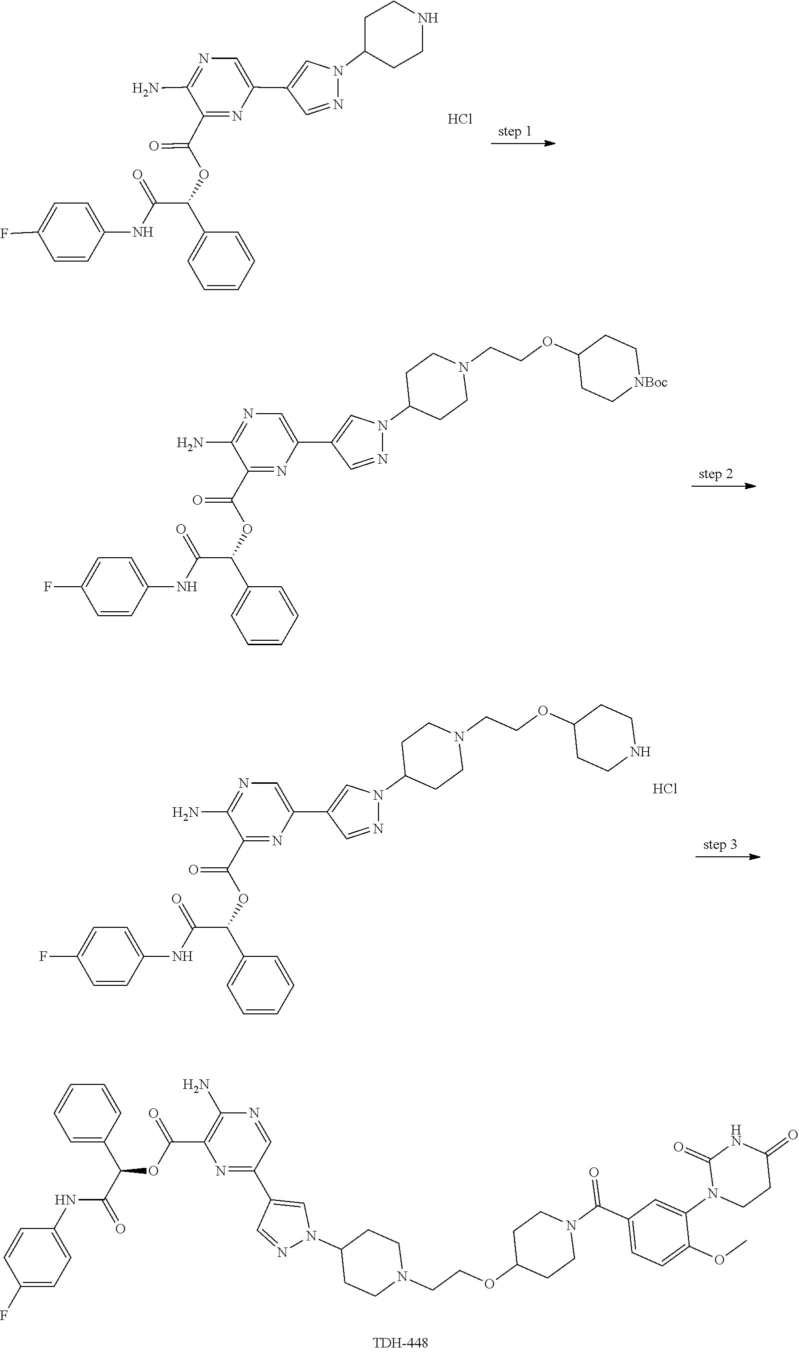 [SMILES: Nc1ncc(-c2cnn(C3CCNCC3)c2)nc1C(=O)O[C@@H](C(=O)Nc1ccc(F)cc1)c1ccccc1 ; Cl ; CC(C)(C)OC(=O)N1CCC(OCCN2CCC(n3cc(-c4cnc(N)c(C(=O)O[C@@H](C(=O)Nc5ccc(F)cc5)c5ccccc5)n4)cn3)CC2)CC1 ; Nc1ncc(-c2cnn(C3CCN(CCOC4CCNCC4)CC3)c2)nc1C(=O)O[C@@H](C(=O)Nc1ccc(F)cc1)c1ccccc1 ; Cl ; COc1ccc(C(=O)N2CCC(OCCN3CCC(n4cc(-c5cnc(N)c(C(=O)O[C@@H](C(=O)Nc6ccc(F)cc6)c6ccccc6)n5)cn4)CC3)CC2)cc1N1CCC(=O)NC1=O]

(R)-2-((4-fluorophenyl)amino)-2-oxo-1-phenylethyl 3-amino-6-(1-(1-(2-((1-(tert-butoxycarbonyl)piperidin-4-yl)oxy)ethyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylate hydrochloride (200 mg, 0.38 mmol, 1.0 eq.), tert-butyl 4-(2-iodoethoxy)piperidine-1-carboxylate (204 mg, 0.58 mmol, 1.5 eq.), and K₂CO₃ (106 mg, 0.77 mmol, 2.0 eq.) were dissolved in DMF (1.5 mL) and reacted overnight at 50° C. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(1-(tert-butoxycarbonyl)piperidin-4-yl)propyl)piperidin-4-yl)-1H-pyrazol-4-yl)pyrazine-2-carboxylate (255 mg, 90%).

¹H NMR (500 MHz, CDCl₃) δ 8.97 (s, 1H), 8.54 (s, 1H), 7.99 (s, 1H), 7.94 (s, 1H), 7.68-7.62 (m, 2H), 7.61-7.54 (m, 2H), 7.48-7.40 (m, 3H), 7.07 (t, J=7.5 Hz, 2H), 6.44 (s, 1H), 6.34 (s, 2H), 4.23 (s, 1H), 3.84-3.75 (m, 2H), 3.70-3.62 (m, 2H), 3.52-3.44 (m, 1H), 3.17-3.05 (m, 4H), 2.73-2.63 (m, 2H), 2.36-2.25 (m, 2H), 2.25-2.15 (m, 2H), 2.12-2.02 (m, 2H), 1.91-1.83 (m, 2H), 1.57-1.51 (m, 2H), 1.48 (s, 9H).

### (R)-2-((4-fluorophenyl)amino)-2-oxo-1-phenylethyl 3-amino-6-(1-(1-(2-(piperidin-4-yloxy)ethyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-(2-((1-(tert-butoxycarbonyl)piperidin-4-yl)oxy)ethyl)piperidin-4-yl)-1H-pyrazol-4-yl)pyrazine-2-carboxylate (255 mg, 0.34 mmol) was dissolved in DCM (2.0 mL). Then, 4 N HCl in dioxane (1.0 mL) was slowly added and reacted at room temperature for 1 hour. After completion of the reaction, the precipitate was filtered to obtain (R)-2-((4-fluorophenyl)amino)-2-oxo-1-phenylethyl 3-amino-6-(1-(1-(2-(piperidin-4-yloxy)ethyl)piperidin-4-yl)-1H-pyrazol-4-yl)pyrazine-2-carboxylate hydrochloride (171 mg, 74%).

¹H NMR (400 MHz, MeOD) δ 8.62 (s, 1H), 8.36-8.28 (m, 1H), 8.09 (s, 1H), 7.78-7.72 (m, 2H), 7.63-7.56 (m, 2H), 7.53-7.44 (m, 3H), 7.08 (t, J=8.6 Hz, 2H), 6.36 (s, 1H), 4.69-4.56 (m, 1H), 3.99-3.89 (m, 2H), 3.89-3.80 (m, 3H), 3.66-3.54 (m, 2H), 3.53-3.47 (m, 2H), 3.46-3.38 (m, 2H), 3.21-3.11 (m, 2H), 2.52-2.38 (m, 4H), 2.18-2.08 (m, 2H), 2.03-1.91 (m, 2H).

### (R)-2-((4-fluorophenyl)amino)-2-oxo-1-phenylethyl 3-amino-6-(1-(1-(2-((1-(3-(2,4-dioxotetrahydropyrimidin-1(2H)-yl)-4-methoxybenzoyl)piperidin-4-yl)oxy)ethyl)piperidin-4-yl)-1H-pyrazol-4-yl)pyrazine-2-carboxylate [step 3, TDH-448]

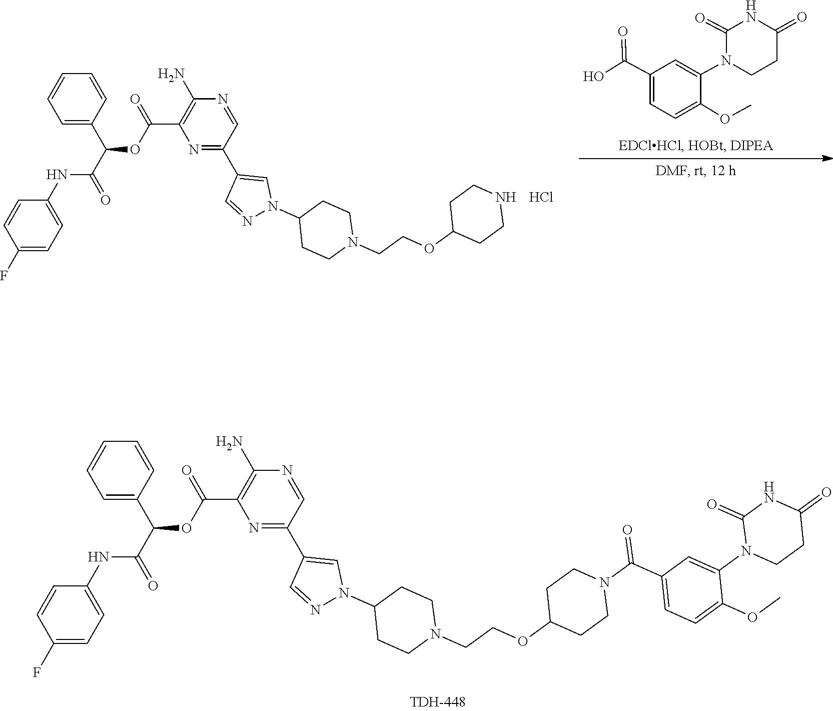 [SMILES: Nc1ncc(-c2cnn(C3CCN(CCOC4CCNCC4)CC3)c2)nc1C(=O)O[C@@H](C(=O)Nc1ccc(F)cc1)c1ccccc1 ; Cl ; COc1ccc(C(=O)O)cc1N1CCC(=O)NC1=O ; COc1ccc(C(=O)N2CCC(OCCN3CCC(n4cc(-c5cnc(N)c(C(=O)O[C@@H](C(=O)Nc6ccc(F)cc6)c6ccccc6)n5)cn4)CC3)CC2)cc1N1CCC(=O)NC1=O]

(R)-2-((4-fluorophenyl)amino)-2-oxo-1-phenylethyl 3-amino-6-(1-(1-(2-(piperidin-4-yloxy)ethyl)piperidin-4-yl)-1H-pyrazol-4-yl)pyrazine-2-carboxylate hydrochloride (20 mg, 0.029 mmol, 1 eq.), 3-(2,4-dioxotetrahydropyrimidin-1(2H)-yl)-4-methoxybenzoic acid (8 mg, 0.029 mmol, 1.0 eq.), EDCI-HCl (6 mg, 0.032 mmol, 1.1 eq.), HOBt (5 mg, 0.032 mmol, 1.1 eq.), and DIPEA (0.03 mL, 0.14 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-((1-(3-(2,4-dioxotetrahydropyrimidin-1(2H)-yl)-4-methoxybenzoyl)piperidin-4-yl)oxy)ethyl)piperidin-4-yl)-1H-pyrazol-4-yl)pyrazine-2-carboxylate (16 mg, 62%).

### Synthesis of (R)-2-((4-fluorophenyl)amino)-2-oxo-1-phenylethyl 3-amino-6-(1-(1-(2-((1-(2-(3-((1-(2,6-dioxopiperidin-3-yl)-2,5-dioxo-2,5-dihydro-1H-pyrrol-3-yl)amino)phenyl)acetyl)piperidin-4-yl)oxy)ethyl)piperidin-4-yl)-1H-pyrazol-4-yl)pyrazine-2-carboxylate [TDH-496]

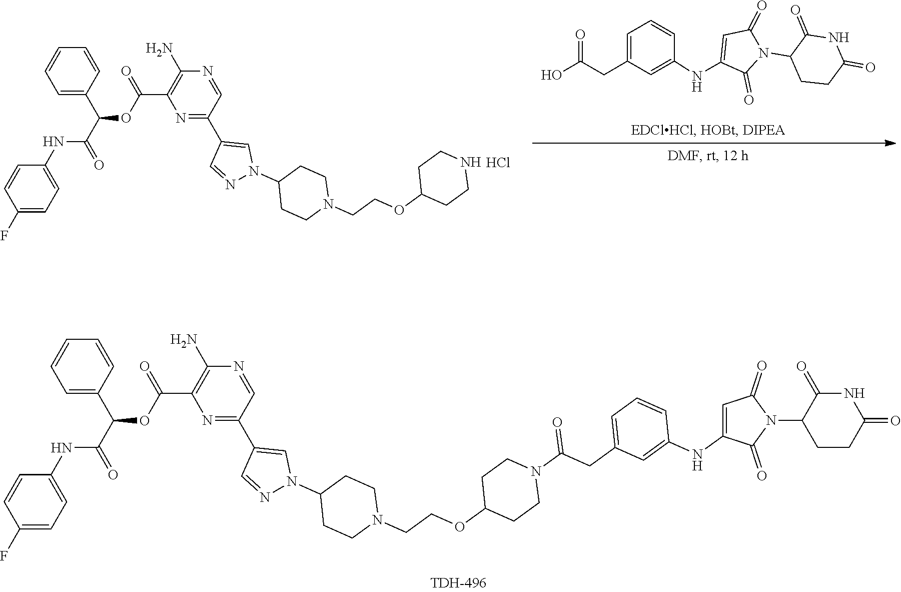 [SMILES: Nc1ncc(-c2cnn(C3CCN(CCOC4CCNCC4)CC3)c2)nc1C(=O)O[C@@H](C(=O)Nc1ccc(F)cc1)c1ccccc1 ; Cl ; O=C(O)Cc1cccc(NC2=CC(=O)N(C3CCC(=O)NC3=O)C2=O)c1 ; Nc1ncc(-c2cnn(C3CCN(CCOC4CCN(C(=O)Cc5cccc(NC6=CC(=O)N(C7CCC(=O)NC7=O)C6=O)c5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yloxy)ethyl)piperidin-4-yl)-1H-pyrazol-4-yl)pyrazine-2-carboxylate hydrochloride (20 mg, 0.029 mmol, 1 eq.), 2-(3-((1-(2,6-dioxopiperidin-3-yl)-2,5-dioxo-2,5-dihydro-1H-pyrrol-3-yl)amino)phenyl)acetic acid (11 mg, 0.029 mmol, 1.0 eq.), EDCI-HCl (6 mg, 0.032 mmol, 1.1 eq.), HOBt (5 mg, 0.032 mmol, 1.1 eq.), and DIPEA (0.03 mL, 0.14 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-((1-(3-(2,4-dioxotetrahydropyrimidin-1(2H)-yl)-4-methoxybenzoyl)piperidin-4-yl)oxy)ethyl)piperidin-4-yl)-1H-pyrazol-4-yl)pyrazine-2-carboxylate (16 mg, 56%).

### Synthesis of (R)-2-((4-fluorophenyl)amino)-2-oxo-1-phenylethyl 3-amino-6-(1-(1-(2-((1-(1-(1-(2,6-dioxopiperidin-3-yl)-2,5-dioxo-2,5-dihydro-1H-pyrrol-3-yl)piperidine-4-carbonyl)piperidin-4-yl)oxy)ethyl)piperidin-4-yl)-1H-pyrazol-4-yl)pyrazine-2-carboxylate [TDH-529]

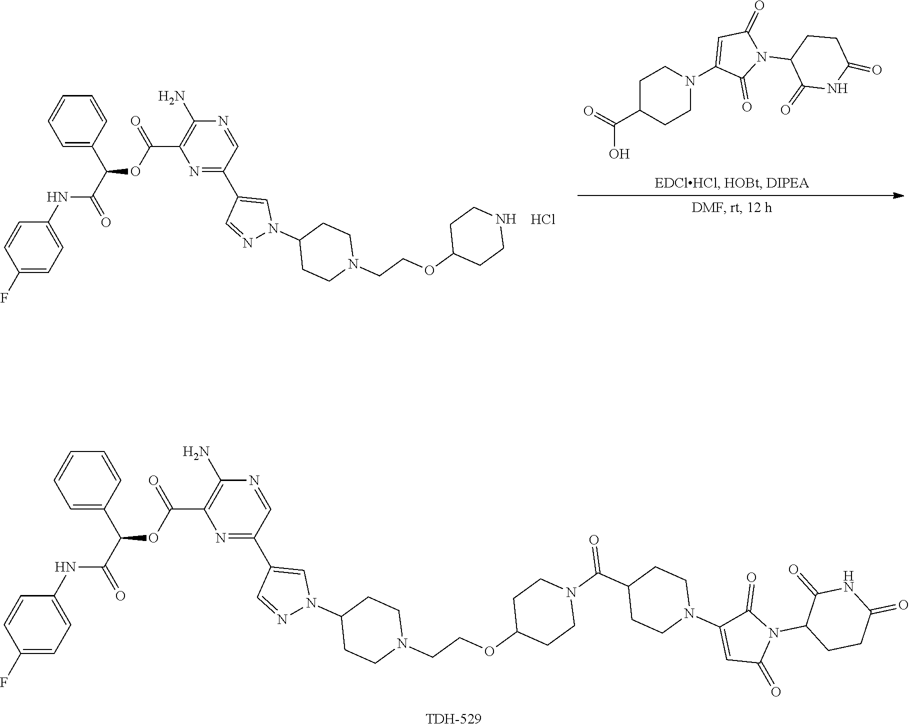 [SMILES: Nc1ncc(-c2cnn(C3CCN(CCOC4CCNCC4)CC3)c2)nc1C(=O)O[C@@H](C(=O)Nc1ccc(F)cc1)c1ccccc1 ; Cl ; O=C1CCC(N2C(=O)C=C(N3CCC(C(=O)O)CC3)C2=O)C(=O)N1 ; Nc1ncc(-c2cnn(C3CCN(CCOC4CCN(C(=O)C5CCN(C6=CC(=O)N(C7CCC(=O)NC7=O)C6=O)CC5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yloxy)ethyl)piperidin-4-yl)-1H-pyrazol-4-yl)pyrazine-2-carboxylate hydrochloride (20 mg, 0.029 mmol, 1 eq.), 1-(1-(2,6-dioxopiperidin-3-yl)-2,5-dioxo-2,5-dihydro-1H-pyrrol-3-yl)piperidine-4-carboxylic acid (10 mg, 0.029 mmol, 1.0 eq.), EDCI-HCl (6 mg, 0.032 mmol, 1.1 eq.), HOBt (5 mg, 0.032 mmol, 1.1 eq.) and DIPEA (0.03 mL, 0.14 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-((1-(1-(1-(2,6-dioxopiperidin-3-yl)-2,5-dioxo-2,5-dihydro-1H-pyrrol-3-yl)piperidine-4-carbonyl)piperidin-4-yl)oxy)ethyl)piperidin-4-yl)-1H-pyrazol-4-yl)pyrazine-2-carboxylate (14 mg, 47%).

### Synthesis of (R)-2-((4-fluorophenyl)amino)-2-oxo-1-phenylethyl 3-amino-6-(1-(1-(2-((1-(4-((2,6-dioxopiperidin-3-yl)carbamoyl)benzoyl)piperidin-4-yl)oxy)ethyl)piperidin-4-yl)-1H-pyrazol-4-yl)pyrazine-2-carboxylate [TDH-538]

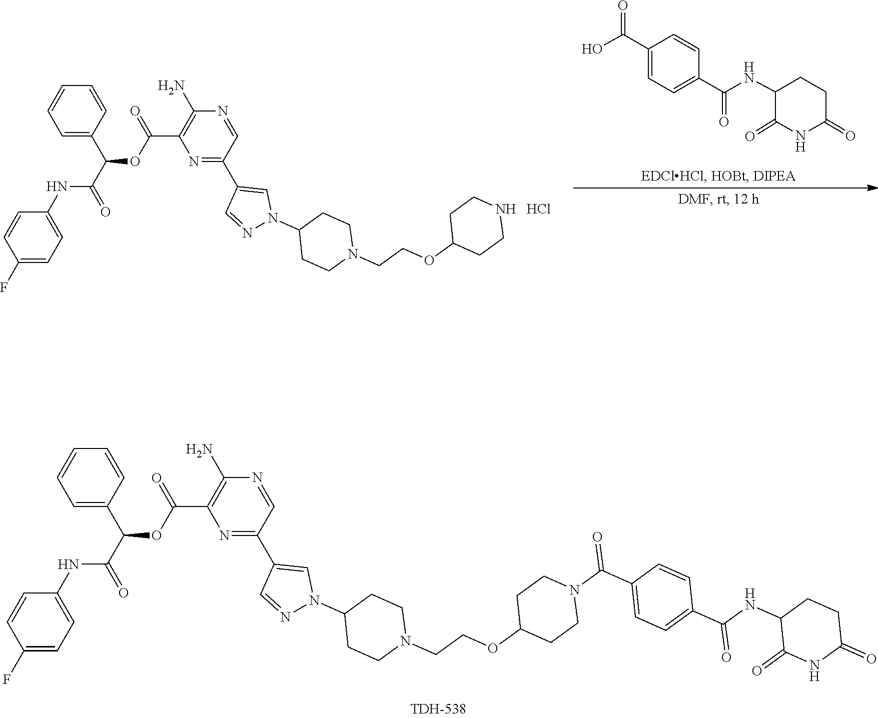 [SMILES: Nc1ncc(-c2cnn(C3CCN(CCOC4CCNCC4)CC3)c2)nc1C(=O)O[C@@H](C(=O)Nc1ccc(F)cc1)c1ccccc1 ; Cl ; O=C1CCC(NC(=O)c2ccc(C(=O)O)cc2)C(=O)N1 ; Nc1ncc(-c2cnn(C3CCN(CCOC4CCN(C(=O)c5ccc(C(=O)NC6CCC(=O)NC6=O)cc5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yloxy)ethyl)piperidin-4-yl)-1H-pyrazol-4-yl)pyrazine-2-carboxylate hydrochloride (20 mg, 0.029 mmol, 1 eq.), 4-((2,6-dioxopiperidin-3-yl)carbamoyl)benzoic acid (9 mg, 0.029 mmol, 1.0 eq.), EDCI-HCl (6 mg, 0.032 mmol, 1.1 eq.), HOBt (5 mg, 0.032 mmol, 1.1 eq.), and DIPEA (0.03 mL, 0.14 mmol, 5 eq.) were dissolved in DMF (1.0 mL), and it was reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-((1-(4-((2,6-dioxopiperidin-3-yl)carbamoyl)benzoyl)piperidin-4-yl)oxy)ethyl)piperidin-4-yl)-1H-pyrazol-4-yl)pyrazine-2-carboxylate (18 mg, 64%).

### Synthesis of (R)-2-((4-fluorophenyl)amino)-2-oxo-1-phenylethyl 3-amino-6-(1-(1-(2-((1-(1-(1-(2,6-dioxopiperidin-3-yl)-2,5-dioxo-2,5-dihydro-1H-pyrrol-3-yl)azetidine-3-carbonyl)piperidin-4-yl)oxy)ethyl)piperidin-4-yl)-1H-pyrazol-4-yl)pyrazine-2-carboxylate [TDH-541]

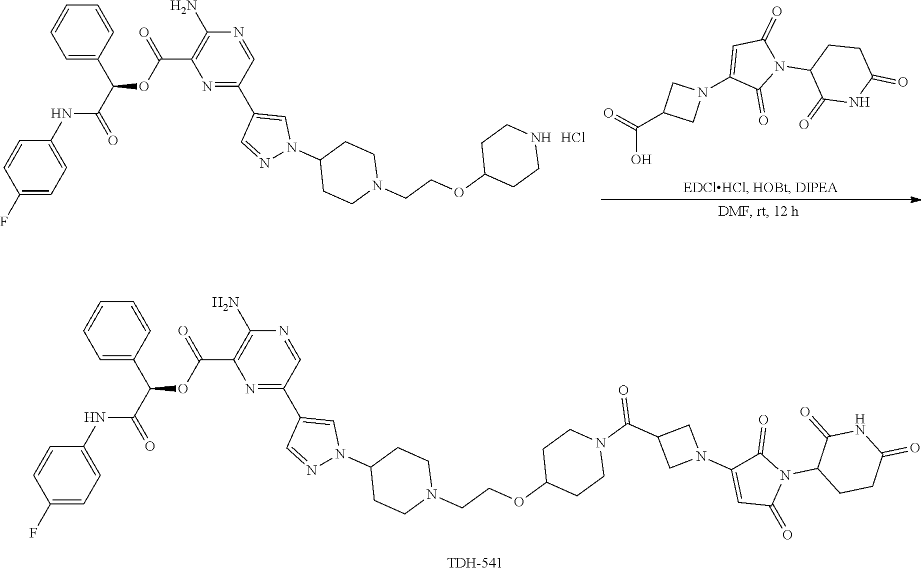 [SMILES: Nc1ncc(-c2cnn(C3CCN(CCOC4CCNCC4)CC3)c2)nc1C(=O)O[C@@H](C(=O)Nc1ccc(F)cc1)c1ccccc1 ; Cl ; O=C1CCC(N2C(=O)C=C(N3CC(C(=O)O)C3)C2=O)C(=O)N1 ; Nc1ncc(-c2cnn(C3CCN(CCOC4CCN(C(=O)C5CN(C6=CC(=O)N(C7CCC(=O)NC7=O)C6=O)C5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yloxy)ethyl)piperidin-4-yl)-1H-pyrazol-4-yl)pyrazine-2-carboxylate hydrochloride (20 mg, 0.029 mmol, 1 eq.), 1-(1-(2,6-dioxopiperidin-3-yl)-2,5-dioxo-2,5-dihydro-1H-pyrrol-3-yl)azetidine-3-carboxylic acid (10 mg, 0.029 mmol, 1.0 eq.), EDCI-HCl (6 mg, 0.032 mmol, 1.1 eq.), HOBt (5 mg, 0.032 mmol, 1.1 eq.) and DIPEA (0.03 mL, 0.14 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-((1-(1-(1-(2,6-dioxopiperidin-3-yl)-2,5-dioxo-2,5-dihydro-1H-pyrrol-3-yl)azetidine-3-carbonyl)piperidin-4-yl)oxy)ethyl)piperidin-4-yl)-1H-pyrazol-4-yl)pyrazine-2-carboxylate (4 mg, 15%).

### Synthesis of (R)-2-((4-fluorophenyl)amino)-2-oxo-1-phenylethyl 3-amino-6-(1-(1-(2-((1-(3-((2,6-dioxopiperidin-3-yl)carbamoyl)benzoyl)piperidin-4-yl)oxy)ethyl)piperidin-4-yl)-1H-pyrazol-4-yl)pyrazine-2-carboxylate [TDH-545]

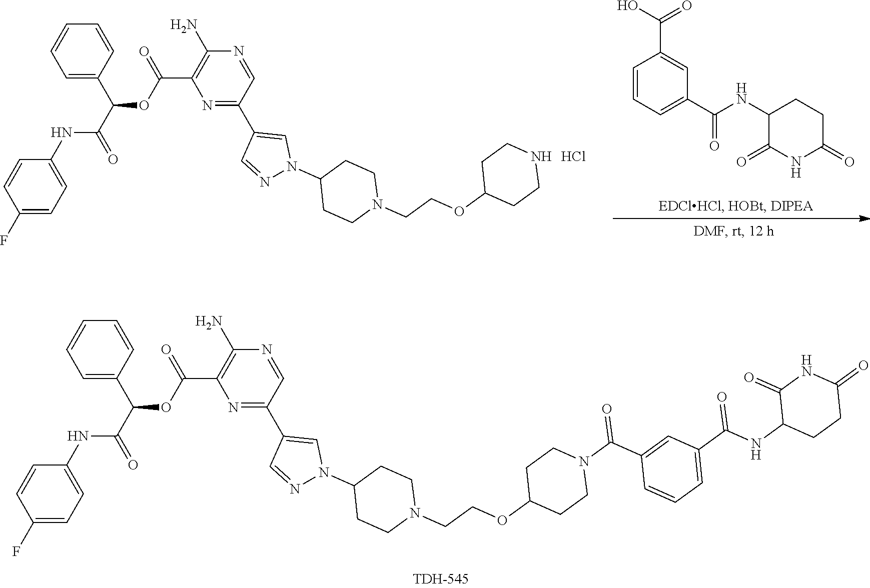 [SMILES: Nc1ncc(-c2cnn(C3CCN(CCOC4CCNCC4)CC3)c2)nc1C(=O)O[C@@H](C(=O)Nc1ccc(F)cc1)c1ccccc1 ; Cl ; O=C1CCC(NC(=O)c2cccc(C(=O)O)c2)C(=O)N1 ; Nc1ncc(-c2cnn(C3CCN(CCOC4CCN(C(=O)c5cccc(C(=O)NC6CCC(=O)NC6=O)c5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yloxy)ethyl)piperidin-4-yl)-1H-pyrazol-4-yl)pyrazine-2-carboxylate hydrochloride (20 mg, 0.029 mmol, 1 eq.), 3-((2,6-dioxopiperidin-3-yl)carbamoyl)benzoic acid (9 mg, 0.029 mmol, 1.0 eq.), EDCI-HCl (6 mg, 0.032 mmol, 1.1 eq.), HOBt (5 mg, 0.032 mmol, 1.1 eq.), and DIPEA (0.03 mL, 0.14 mmol, 5 eq.) were dissolved in DMF (1.0 mL), and it was reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, ((R)-2-((4-fluorophenyl)amino)-2-oxo-1-phenylethyl 3-amino-6-(1-(1-(2-((1-(3-((2,6-dioxopiperidin-3-yl)carbamoyl)benzoyl)piperidin-4-yl)oxy)ethyl)piperidin-4-yl)-1H-pyrazol-4-yl)pyrazine-2-carboxylate (19 mg, 68%).

### Synthesis of (R)-2-((4-fluorophenyl)amino)-2-oxo-1-phenylethyl 3-amino-6-(1-(1-(2-((1-(2-(2,6-dioxopiperidin-3-yl)-1-oxoisoindoline-5-carbonyl)piperidin-4-yl)oxy)ethyl)piperidin-4-yl)-1H-pyrazol-4-yl)pyrazine-2-carboxylate [TDH-549]

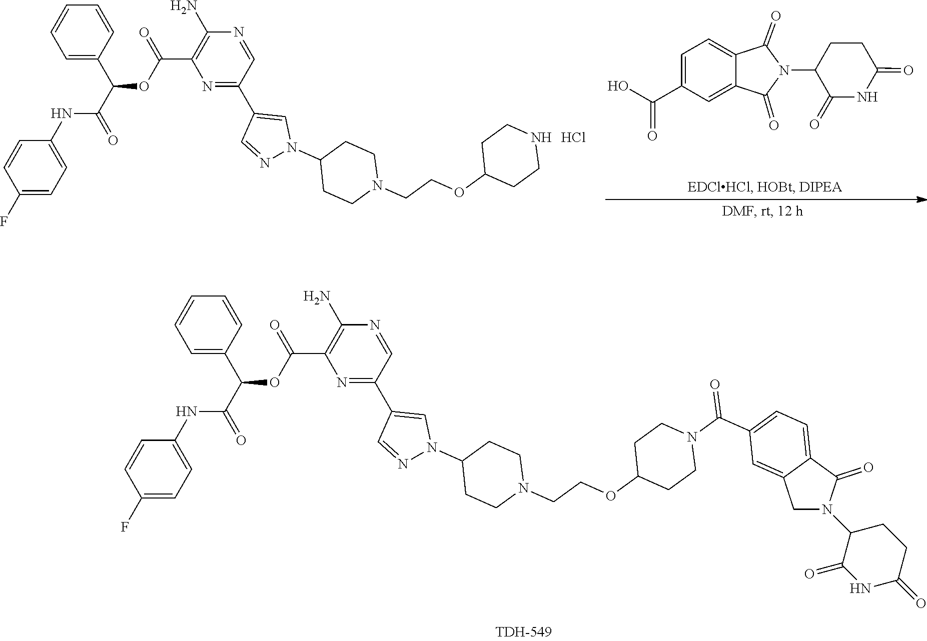 [SMILES: Nc1ncc(-c2cnn(C3CCN(CCOC4CCNCC4)CC3)c2)nc1C(=O)O[C@@H](C(=O)Nc1ccc(F)cc1)c1ccccc1 ; Cl ; O=C1CCC(N2C(=O)c3ccc(C(=O)O)cc3C2=O)C(=O)N1 ; Nc1ncc(-c2cnn(C3CCN(CCOC4CCN(C(=O)c5ccc6c(c5)CN(C5CCC(=O)NC5=O)C6=O)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yloxy)ethyl)piperidin-4-yl)-1H-pyrazol-4-yl)pyrazine-2-carboxylate hydrochloride (20 mg, 0.029 mmol, 1 eq.), 2-(2,6-dioxopiperidin-3-yl)-1-oxoisoindoline-5-carboxylic acid (9 mg, 0.029 mmol, 1.0 eq.), EDCI-HCl (6 mg, 0.032 mmol, 1.1 eq.), HOBt (5 mg, 0.032 mmol, 1.1 eq.), and DIPEA (0.03 mL, 0.14 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-((1-(2-(2,6-dioxopiperidin-3-yl)-1-oxoisoindoline-5-carbonyl)piperidin-4-yl)oxy)ethyl)piperidin-4-yl)-1H-pyrazol-4-yl)pyrazine-2-carboxylate (22 mg, 78%).

### Synthesis of (R)-2-((4-fluorophenyl)amino)-2-oxo-1-phenylethyl 3-amino-6-(1-(1-(2-((1-(3-(2,4-dioxotetrahydropyrimidin-1(2H)-yl)benzofuran-6-carbonyl)piperidin-4-yl)oxy)ethyl)piperidin-4-yl)-1H-pyrazol-4-yl)pyrazine-2-carboxylate [TDH-554]

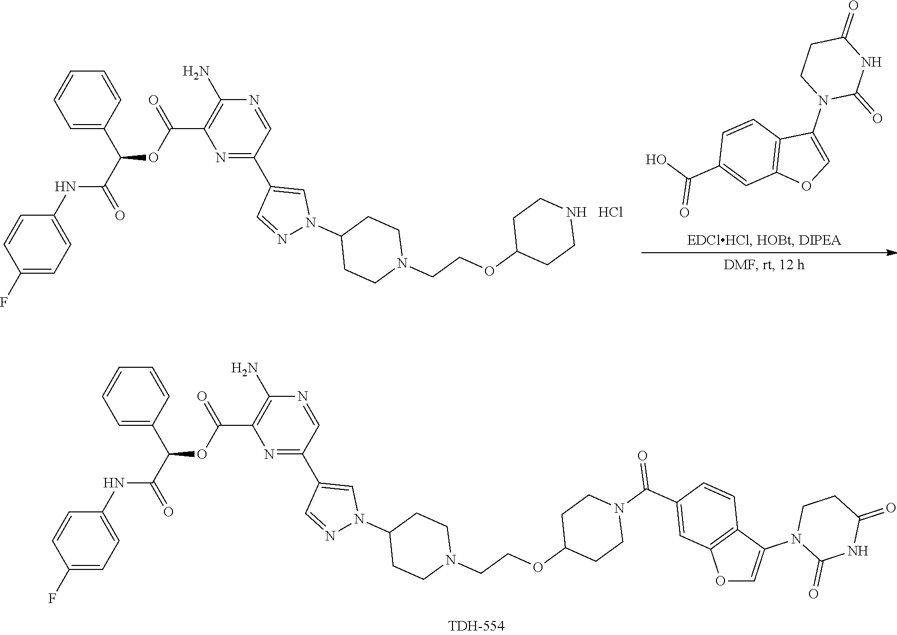 [SMILES: Nc1ncc(-c2cnn(C3CCN(CCOC4CCNCC4)CC3)c2)nc1C(=O)O[C@@H](C(=O)Nc1ccc(F)cc1)c1ccccc1 ; Cl ; O=C1CCN(c2coc3cc(C(=O)O)ccc23)C(=O)N1 ; Nc1ncc(-c2cnn(C3CCN(CCOC4CCN(C(=O)c5ccc6c(N7CCC(=O)NC7=O)coc6c5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(piperidin-4-yloxy)ethyl)piperidin-4-yl)-1H-pyrazol-4-yl)pyrazine-2-carboxylate hydrochloride (20 mg, 0.029 mmol, 1 eq.), 3-(2,4-dioxotetrahydropyrimidin-1(2H)-yl)benzofuran-6-carboxylic acid (9 mg, 0.029 mmol, 1.0 eq.), EDCI-HCl (6 mg, 0.032 mmol, 1.1 eq.), HOBt (5 mg, 0.032 mmol, 1.1 eq.), and DIPEA (0.03 mL, 0.14 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-((1-(3-(2,4-dioxotetrahydropyrimidin-1(2H)-yl)benzofuran-6-carbonyl)piperidin-4-yl)oxy)ethyl)piperidin-4-yl)-1H-pyrazol-4-yl)pyrazine-2-carboxylate (18 mg, 65%).

### Synthesis of (R)-2-((4-fluorophenyl)amino)-2-oxo-1-phenylethyl 3-amino-6-(1-(1-(2-(1-(1-(2-((2-(2,6-dioxopiperidin-3-yl)-1,3-dioxoisoindolin-5-yl)oxy)acetyl)piperidin-4-yl)-1H-1,2,3-triazol-4-yl)ethyl)piperidin-4-yl)-1H-pyrazol-4-yl)pyrazine-2-carboxylate [TDH-451]

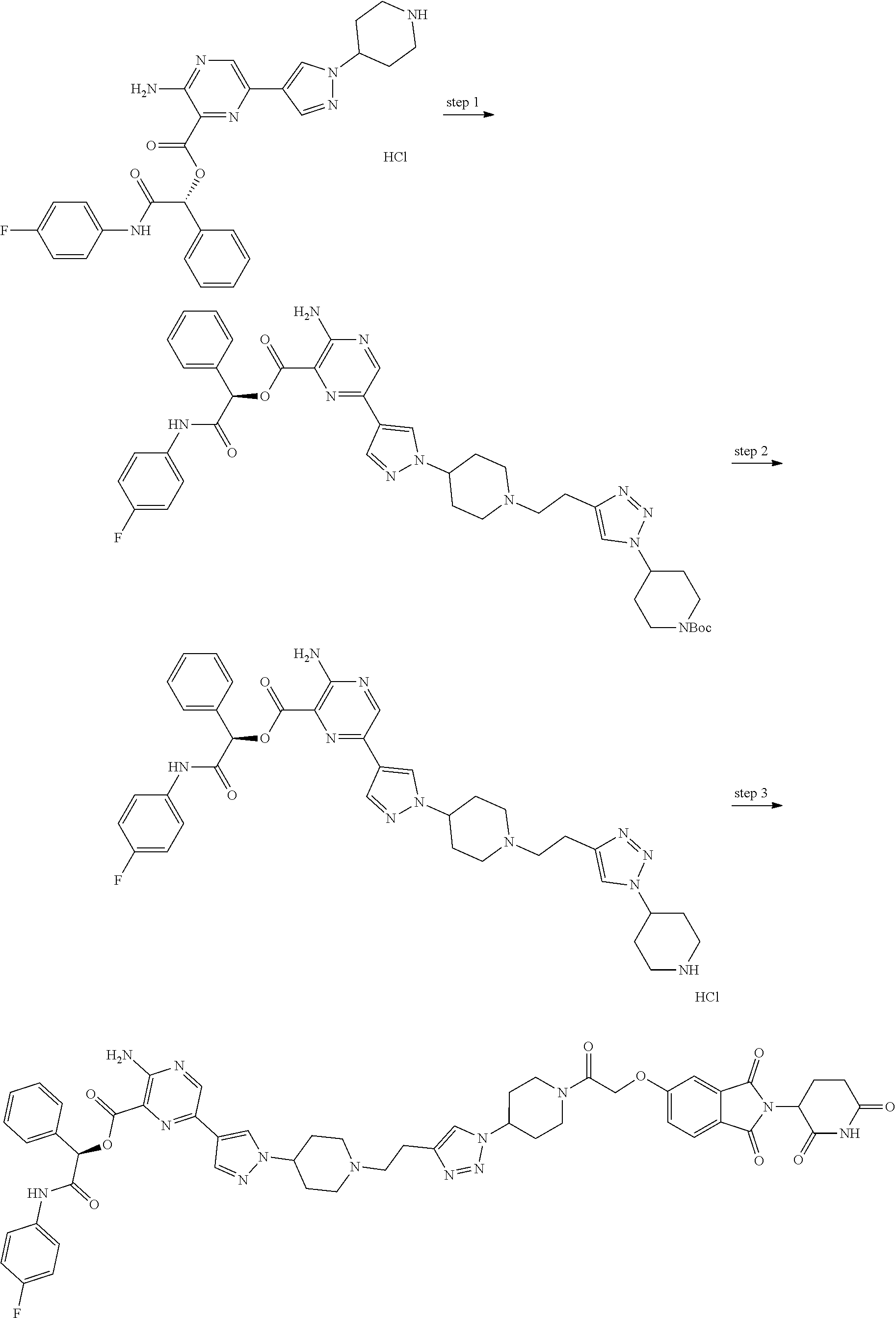

### (R)-2-((4-fluorophenyl)amino)-2-oxo-1-phenylethyl 3-amino-6-(1-(1-(2-(1-(1-(tert-butoxycarbonyl)piperidin-4-yl)-1H-1,2,3-triazol-4-yl)ethyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylate hydrochloride (200 mg, 0.38 mmol, 1.0 eq.), tert-butyl 4-(4-(2-(tosyloxy)ethyl)-1H-1,2,3-triazol-1-yl)piperidine-1-Carboxylate (260 mg, 0.58 mmol, 1.5 eq.) and K₂CO₃ (106 mg, 0.77 mmol, 2.0 eq.) were dissolved in DMF (1.5 mL) and reacted at 50° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(1-(tert-butoxycarbonyl)piperidin-4-yl)-1H-1,2,3-triazol-4-yl)ethyl)piperidin-4-yl)-1H-pyrazol-4-yl)pyrazine-2-carboxylate (98 mg, 31%).

¹H NMR (400 MHz, CDCl₃) δ 8.99 (s, 1H), 8.54 (s, 1H), 8.00 (s, 1H), 7.95 (s, 1H), 7.70-7.63 (m, 2H), 7.63-7.54 (m, 2H), 7.48-7.38 (m, 4H), 7.09-7.02 (m, 2H), 6.44 (s, 1H), 6.35 (s, 2H), 4.64-4.54 (m, 1H), 4.34-4.19 (m, 3H), 3.23-3.13 (m, 2H), 3.03-2.92 (m, 4H), 2.85-2.74 (m, 2H), 2.31-2.17 (m, 6H), 2.13-2.05 (m, 2H), 2.00-1.92 (m, 2H), 1.50 (s, 9H).

### (R)-2-((4-fluorophenyl)amino)-2-oxo-1-phenylethyl 3-amino-6-(1-(1-(2-(1-(piperidin-4-yl)-1H-1,2,3-triazol-4-yl)ethyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-(2-(1-(1-(tert-butoxycarbonyl)piperidin-4-yl)-1H-1,2,3-triazol-4-yl)ethyl)piperidin-4-yl)-1H-pyrazol-4-yl)pyrazine-2-carboxylate (98 mg, 0.12 mmol) was dissolved in DCM (1.0 mL). Then, 4 N HCl in dioxane (0.5 mL) was slowly added, and reacted at room temperature for 1 hour. After completion of the reaction, the precipitate was filtered to obtain (R)-2-((4-fluorophenyl)amino)-2-oxo-1-phenylethyl 3-amino-6-(1-(1-(2-(1-(piperidin-4-yl)-1H-1,2,3-triazol-4-yl)ethyl)piperidin-4-yl)-1H-pyrazol-4-yl)pyrazine-2-carboxylate hydrochloride (70 mg, 80%).

¹H NMR (400 MHz, MeOD) δ 8.62 (s, 1H), 8.30 (s, 1H), 8.11 (s, 1H), 8.09 (s, 1H), 7.76-7.72 (m, 2H), 7.62-7.56 (m, 2H), 7.52-7.42 (m, 3H), 7.11-7.04 (m, 2H), 6.36 (s, 1H), 3.95-3.87 (m, 2H), 3.66-3.55 (m, 5H), 3.32-3.23 (m, 7H), 2.50-2.37 (m, 8H).

### (R)-2-((4-fluorophenyl)amino)-2-oxo-1-phenylethyl 3-amino-6-(1-(1-(2-(1-(1-(2-((2-(2,6-dioxopiperidin-3-yl)-1,3-dioxoisoindolin-5-yl)oxy)acetyl)piperidin-4-yl)-1H-1,2,3-triazol-4-yl)ethyl)piperidin-4-yl)-1H-pyrazol-4-yl)pyrazine-2-carboxylate [step 3, TDH-451]

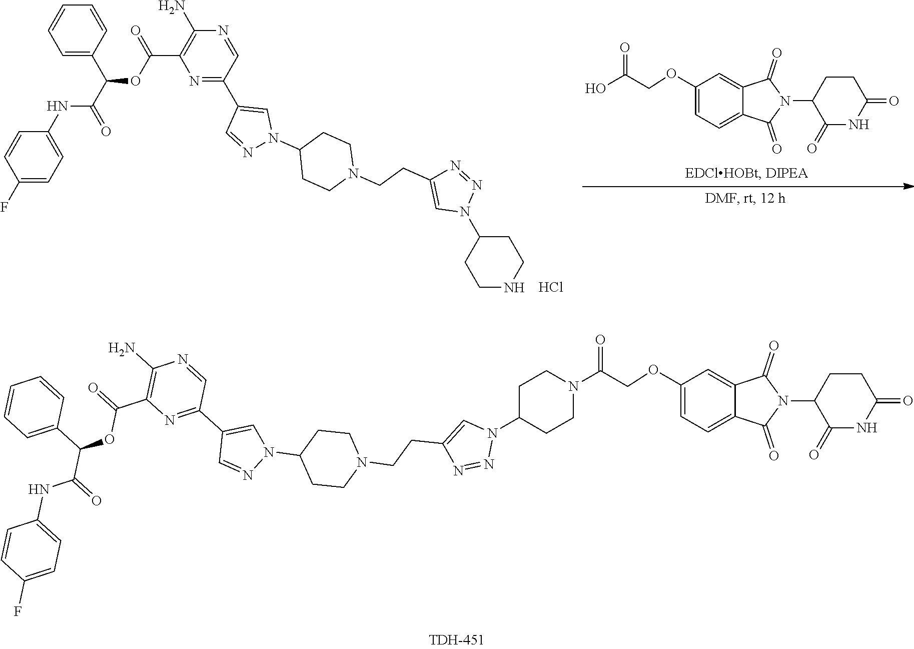 [SMILES: Nc1ncc(-c2cnn(C3CCN(CCc4cn(C5CCNCC5)nn4)CC3)c2)nc1C(=O)O[C@@H](C(=O)Nc1ccc(F)cc1)c1ccccc1 ; Cl ; O=C(O)COc1ccc2c(c1)C(=O)N(C1CCC(=O)NC1=O)C2=O ; Nc1ncc(-c2cnn(C3CCN(CCc4cn(C5CCN(C(=O)COc6ccc7c(c6)C(=O)N(C6CCC(=O)NC6=O)C7=O)CC5)nn4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(1-(piperidin-4-yl)-1H-1,2,3-triazol-4-yl)ethyl)piperidin-4-yl)-1H-pyrazol-4-yl)pyrazine-2-carboxylate hydrochloride (20 mg, 0.027 mmol, 1 eq.), 2-((2-(2,6-dioxopiperidin-3-yl)-1,3-dioxoisoindolin-5-yl)oxy)acetic acid (10 mg, 0.027 mmol, 1.0 eq.), EDCI-HCl (6 mg, 0.030 mmol, 1.1 eq.), HOBt (5 mg, 0.030 mmol, 1.1 eq.), and DIPEA (0.03 mL, 0.14 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(1-(2-((2-(2,6-dioxopiperidin-3-yl)-1,3-dioxoisoindolin-5-yl)oxy)acetyl)piperidin-4-yl)-1H-1,2,3-triazol-4-yl)ethyl)piperidin-4-yl)-1H-pyrazol-4-yl)pyrazine-2-carboxylate (15 mg, 57%).

### Synthesis of ((R)-2-((4-fluorophenyl)amino)-2-oxo-1-phenylethyl 3-amino-6-(1-(1-(2-(1-(1-((2-(2,6-dioxopiperidin-3-yl)-1,3-dioxoisoindolin-5-yl)glycyl)piperidin-4-yl)-1H-1,2,3-triazol-4-yl)ethyl)piperidin-4-yl)-1H-pyrazol-4-yl)pyrazine-2-carboxylate [TDH-452]

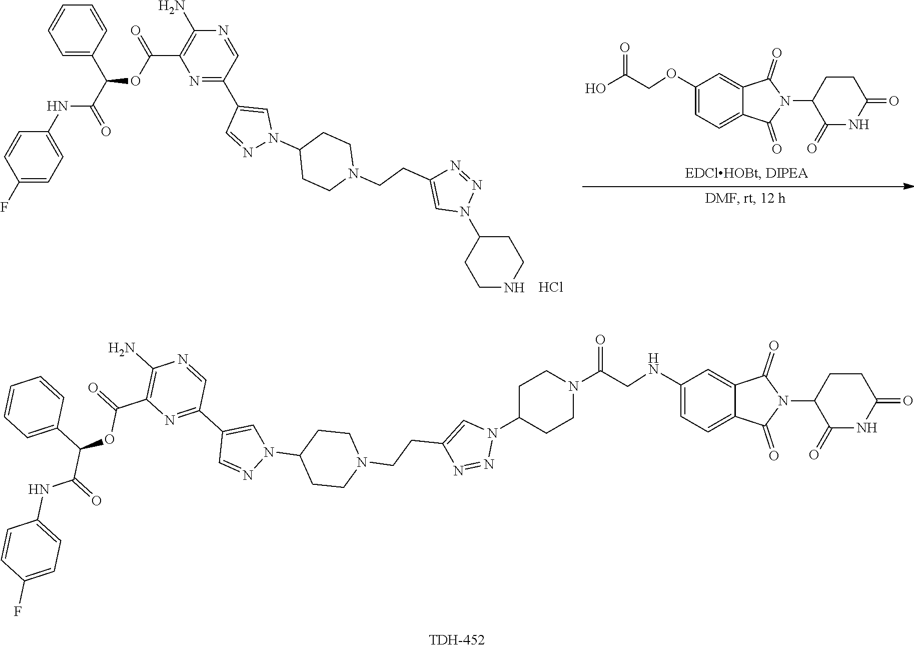 [SMILES: Nc1ncc(-c2cnn(C3CCN(CCc4cn(C5CCNCC5)nn4)CC3)c2)nc1C(=O)O[C@@H](C(=O)Nc1ccc(F)cc1)c1ccccc1 ; Cl ; O=C(O)COc1ccc2c(c1)C(=O)N(C1CCC(=O)NC1=O)C2=O ; Nc1ncc(-c2cnn(C3CCN(CCc4cn(C5CCN(C(=O)CNc6ccc7c(c6)C(=O)N(C6CCC(=O)NC6=O)C7=O)CC5)nn4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(2-(1-(piperidin-4-yl)-1H-1,2,3-triazol-4-yl)ethyl)piperidin-4-yl)-1H-pyrazol-4-yl)pyrazine-2-carboxylate hydrochloride (20 mg, 0.027 mmol, 1 eq.), (2-(2,6-dioxopiperidin-3-yl)-1,3-dioxoisoindolin-5-yl)glycine (9 mg, 0.027 mmol, 1.0 eq.), EDCI-HCl (6 mg, 0.030 mmol, 1.1 eq.), HOBt (5 mg, 0.030 mmol, 1.1 eq.) and DIPEA (0.03 mL, 0.14 mmol, 5 eq.) were dissolved in DMF (1.0 mL), and the mixture was reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(2-(1-(1-((2-(2,6-dioxopiperidin-3-yl)-1,3-dioxoisoindolin-5-yl)glycyl)piperidin-4-yl)-1H-1,2,3-triazol-4-yl)ethyl)piperidin-4-yl)-1H-pyrazol-4-yl)pyrazine-2-carboxylate (17 mg, 63%).

### Synthesis of (R)-2-((4-fluorophenyl)amino)-2-oxo-1-phenylethyl 3-amino-6-(1-(1-(3-(5-(2-((2-(2,6-dioxopiperidin-3-yl)-1,3-dioxoisoindolin-5-yl)oxy)acetyl)-2,5-diazabicyclo[2.2.1]heptan-2-yl)propyl)piperidin-4-yl)-1H-pyrazol-4-yl)pyrazine-2-carboxylate [TDH-453]

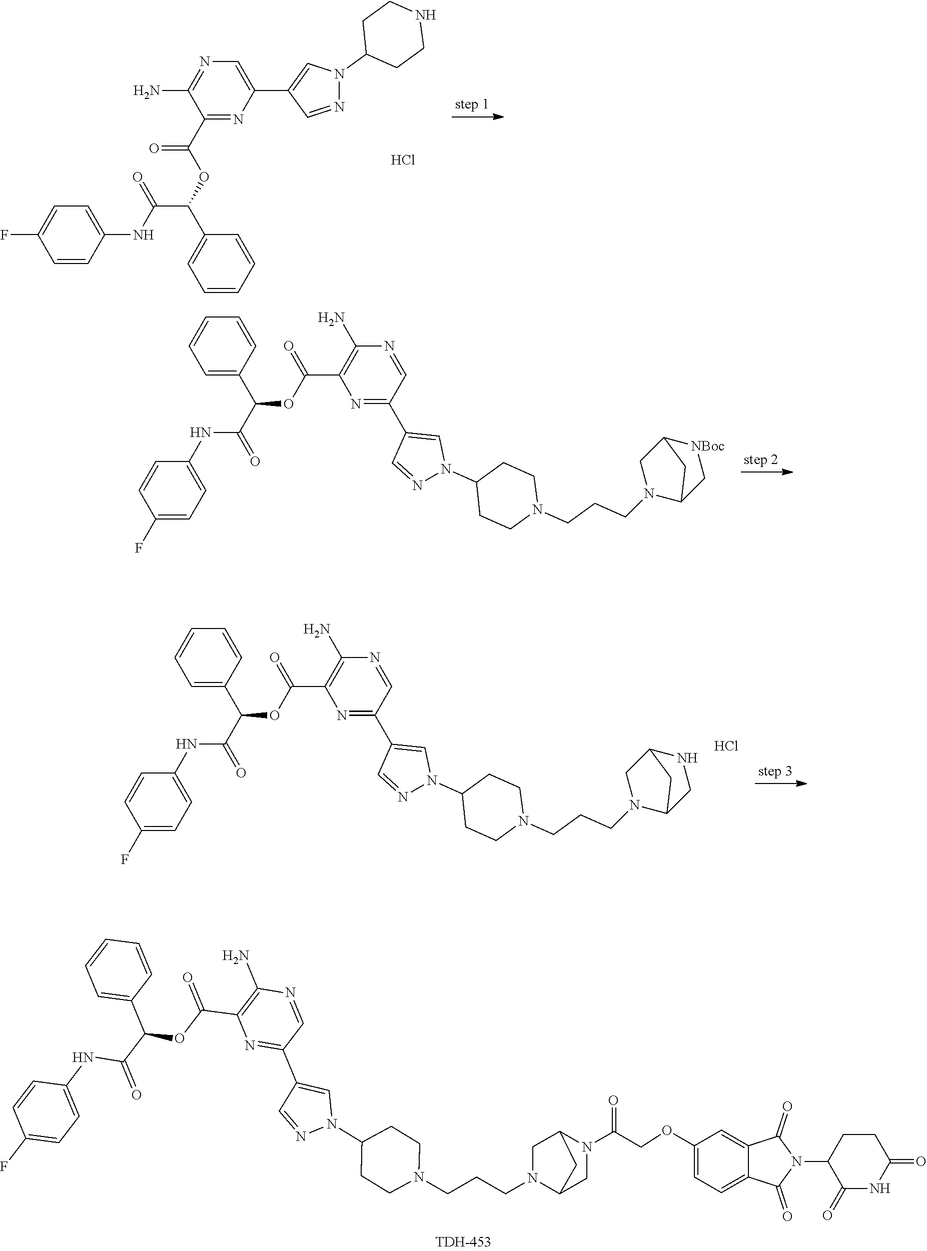

### Tert-butyl 5-(3-(4-(4-(5-amino-6-(((R)-2-((4-fluorophenyl)amino)-2-oxo-1-phenylethoxy)carbonyl)pyrazin-2-yl)-1H-pyrazol-1-yl)piperidin-1-yl)propyl)-2,5-diazabicyclo[2.2.1]heptane-2-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylate hydrochloride (79 mg, 0.14 mmol, 1.0 eq.), tert-butyl 5-(3-iodopropyl)-2,5-diazabicyclo[2.2.1]heptane-2-carboxylate (79 mg, 0.22 mmol, 1.5 eq.), and K₂CO₃ (40 mg, 0.29 mmol, 2.0 eq.) were dissolved in DMF (1.0 mL) and reacted at 50° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, tert-butyl 5-(3-(4-(4-(5-amino-6-(((R)-2-((4-fluorophenyl))amino)-2-oxo-1-phenylethoxy)carbonyl)pyrazin-2-yl)-1H-pyrazol-1-yl)piperidin-1-yl)propyl)-2,5-diazabicyclo[2.2.1]heptane-2-carboxylate (36 mg, 34%).

¹H NMR (500 MHz, CDCl₃) δ 9.06 (s, 1H), 8.56-8.53 (m, 1H), 8.02-7.98 (m, 1H), 7.96-7.93 (m, 1H), 7.69-7.64 (m, 2H), 7.62-7.55 (m, 2H), 7.48-7.38 (m, 3H), 7.10-7.01 (m, 2H), 6.47-6.43 (m, 1H), 6.35 (s, 2H), 4.38 (s, 0.5H), 4.25 (s, 0.5H), 4.24-4.19 (m, 1H), 3.62-3.48 (m, 2H), 3.21-3.15 (m, 1H), 3.13-3.06 (m, 2H), 3.05-2.99 (m, 1H), 2.97-2.91 (m, 0.5H), 2.85-2.78 (m, 0.5H), 2.70-2.57 (m, 2H), 2.52-2.44 (m, 2H), 2.26-2.12 (m, 4H), 2.12-2.02 (m, 2H), 2.00-1.91 (m, 1H), 1.91-1.81 (m, 2H), 1.48 (s, 9H).

### (R)-2-((4-fluorophenyl)amino)-2-oxo-1-phenylethyl 6-(1-(1-(3-(2,5-diazabicyclo[2.2.1]heptan-2-yl)propyl)piperidin-4-yl)-1H-pyrazol-4-yl)-3-aminopyrazine-2-carboxylate hydrochloride (step 2)

Tert-butyl 5-(3-(4-(4-(5-amino-6-(((R)-2-((4-fluorophenyl)amino)-2-oxo-1-phenylethoxy)carbonyl)pyrazin-2-yl)-1H-pyrazol-1-yl)piperidin-1-yl)propyl)-2,5-diazabicyclo[2.2.1]heptane-2-carboxylate (36 mg, 0.047 mmol) was dissolved in DCM (1.0 mL). Then, 4 N HCl in dioxane (0.5 mL) was slowly added, and reacted at room temperature for 1 hour. After completion of the reaction, the precipitate was filtered to obtain (R)-2-((4-fluorophenyl)amino)-2-oxo-1-phenylethyl 6-(1-(1-(3-(2,5-diazabicyclo[2.2.1]heptan-2-yl)propyl)piperidin-4-yl)-1H-pyrazol-4-yl)-3-aminopyrazine-2-carboxylate hydrochloride (29 mg, 89%).

¹H NMR (400 MHz, MeOD) δ 8.62 (s, 1H), 8.30 (s, 1H), 8.09 (s, 1H), 7.76-7.72 (m, 2H), 7.62-7.56 (m, 2H), 7.52-7.43 (m, 3H), 7.11-7.05 (m, 2H), 6.36 (s, 1H), 4.75-4.71 (m, 1H), 4.70-4.60 (m, 2H), 4.08-4.02 (m, 1H), 3.89-3.81 (m, 2H), 3.64-3.56 (m, 3H), 3.50-3.43 (m, 2H), 3.42-3.36 (m, 2H), 2.76-2.58 (m, 2H), 2.55-2.42 (m, 4H), 2.42-2.29 (m, 4H).

### Synthesis of (R)-2-((4-fluorophenyl)amino)-2-oxo-1-phenylethyl 3-amino-6-(1-(1-(3-(5-(2-((2-(2,6-dioxopiperidin-3-yl)-1,3-dioxoisoindolin-5-yl)oxy)acetyl)-2,5-diazabicyclo[2.2.1]heptan-2-yl)propyl)piperidin-4-yl)-1H-pyrazol-4-yl)pyrazine-2-carboxylate [step 3, TDH-453]

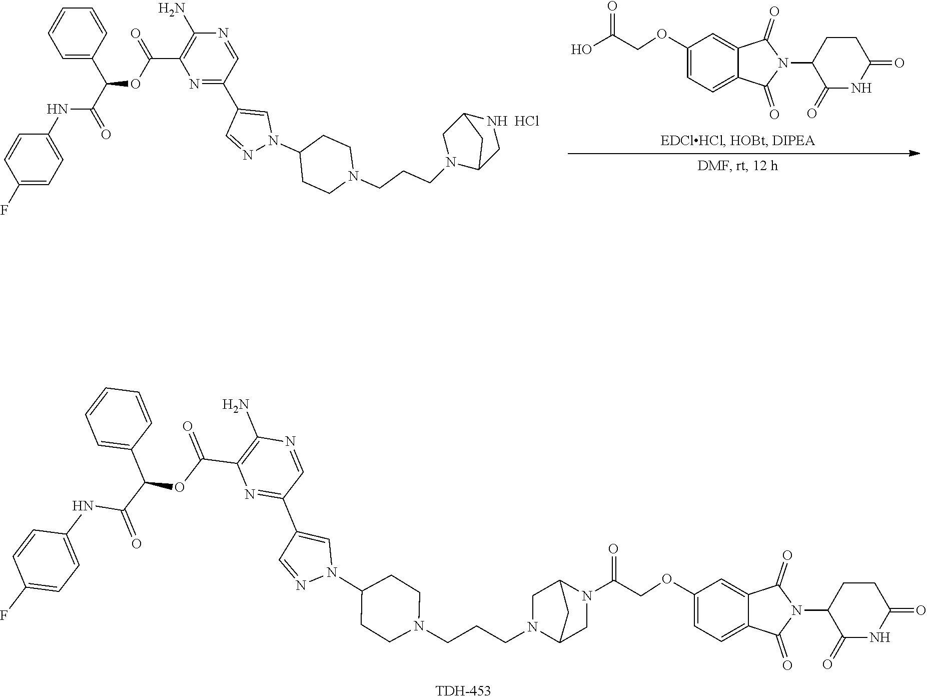 [SMILES: Nc1ncc(-c2cnn(C3CCN(CCCN4CC5CC4CN5)CC3)c2)nc1C(=O)O[C@@H](C(=O)Nc1ccc(F)cc1)c1ccccc1 ; Cl ; O=C(O)COc1ccc2c(c1)C(=O)N(C1CCC(=O)NC1=O)C2=O ; Nc1ncc(-c2cnn(C3CCN(CCCN4CC5CC4CN5C(=O)COc4ccc5c(c4)C(=O)N(C4CCC(=O)NC4=O)C5=O)CC3)c2)nc1C(=O)O[C@@H](C(=O)Nc1ccc(F)cc1)c1ccccc1]

(R)-2-((4-fluorophenyl)amino)-2-oxo-1-phenylethyl 6-(1-(1-(3-(2,5-diazabicyclo[2.2.1]heptan-2-yl)propyl)piperidin-4-yl)-1H-pyrazol-4-yl)-3-aminopyrazine-2-carboxylate hydrochloride (15 mg, 0.022 mmol, 1 eq.), 2-((2-(2,6-dioxopiperidin-3-yl)-1,3-dioxoisoindolin-5-yl)oxy)acetic acid (8 mg, 0.022 mmol, 1.0 eq.), EDCI-HCl (5 mg, 0.024 mmol, 1.1 eq.), HOBt (4 mg, 0.024 mmol, 1.1 eq.) and DIPEA (0.02 mL, 0.11 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(5-(2-((2-(2,6-dioxopiperidin-3-yl)-1,3-dioxoisoindolin-5-yl)oxy)acetyl)-2,5-diazabicyclo[2.2.1] Heptan-2-yl)propyl)piperidin-4-yl)-1H-pyrazol-4-yl)pyrazine-2-carboxylate (12 mg, 56%).

### Synthesis of (R)-2-((4-fluorophenyl)amino)-2-oxo-1-phenylethyl 3-amino-6-(1-(1-(3-(5-((2-(2,6-dioxopiperidin-3-yl)-1,3-dioxoisoindolin-5-yl)glycyl)-2,5-diazabicyclo[2.2.1]heptan-2-yl)propyl)piperidin-4-yl)-1H-pyrazol-4-yl)pyrazine-2-carboxylate [TDH-454]

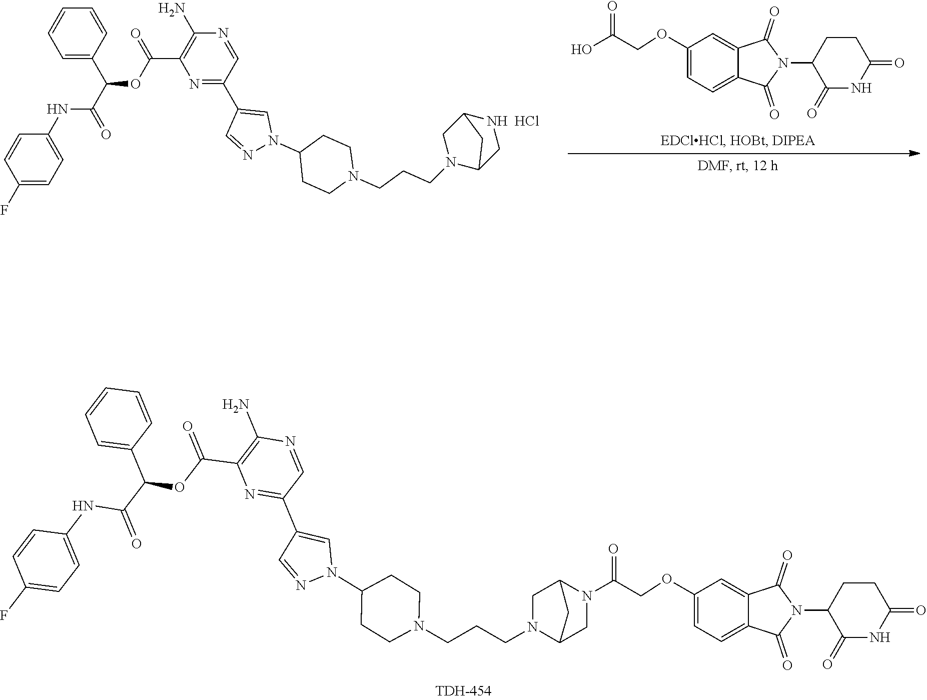 [SMILES: Nc1ncc(-c2cnn(C3CCN(CCCN4CC5CC4CN5)CC3)c2)nc1C(=O)O[C@@H](C(=O)Nc1ccc(F)cc1)c1ccccc1 ; Cl ; O=C(O)COc1ccc2c(c1)C(=O)N(C1CCC(=O)NC1=O)C2=O ; Nc1ncc(-c2cnn(C3CCN(CCCN4CC5CC4CN5C(=O)COc4ccc5c(c4)C(=O)N(C4CCC(=O)NC4=O)C5=O)CC3)c2)nc1C(=O)O[C@@H](C(=O)Nc1ccc(F)cc1)c1ccccc1]

(R)-2-((4-fluorophenyl)amino)-2-oxo-1-phenylethyl 6-(1-(1-(3-(2,5-diazabicyclo[2.2.1]heptan-2-yl)propyl)piperidin-4-yl)-1H-pyrazol-4-yl)-3-aminopyrazine-2-carboxylate hydrochloride (15 mg, 0.022 mmol, 1 eq.), (2-(2,6-dioxopiperidin-3-yl)-1,3-dioxoisoindolin-5-yl)glycine (8 mg, 0.022 mmol, 1.0 eq.), EDCI-HCl (5 mg, 0.024 mmol, 1.1 eq.), HOBt (4 mg, 0.024 mmol, 1.1 eq.) and DIPEA (0.02 mL, 0.11 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(5-((2-(2,6-dioxopiperidin-3-yl)-1,3-dioxoisoindolin-5-yl)glycyl)-2,5-diazabicyclo[2.2.1]heptan-2-yl)propyl)piperidin-4-yl)-1H-pyrazol-4-yl)pyrazine-2-carboxylate (10 mg, 47%).

### Synthesis of (R)-2-((4-fluorophenyl)amino)-2-oxo-1-phenylethyl 3-amino-6-(1-(1-(3-(5-(2-((2-(2,6-dioxopiperidin-3-yl)-1,3-dioxoisoindolin-5-yl)oxy)acetyl)hexahydropyrrolo[3,4-c]pyrrol-2(1H)-yl)propyl)piperidin-4-yl)-1H-pyrazol-4-yl)pyrazine-2-carboxylate [TDH-466]

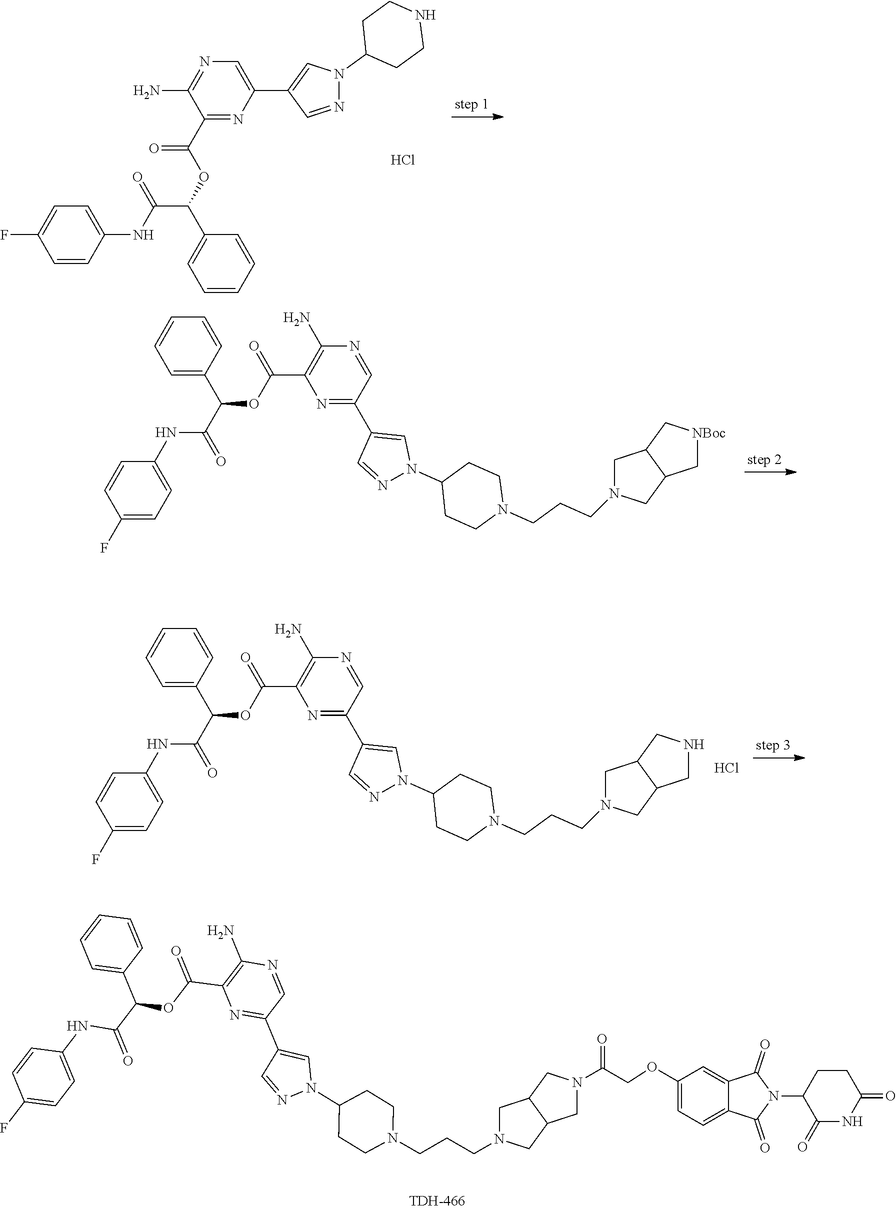

### Tert-butyl 5-(3-(4-(4-(5-amino-6-(((R)-2-((4-fluorophenyl)amino)-2-oxo-1-phenylethoxy)carbonyl)pyrazin-2-yl)-1H-pyrazol-1-yl)piperidin-1-yl)propyl)hexahydropyrrolo[3,4-c]pyrrole-2(1H)-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylate hydrochloride (30 mg, 0.06 mmol, 1.0 eq.), tert-butyl 5-(3-iodopropyl)hexahydropyrrolo[3,4-c]pyrrole-2(1H)-carboxylate (34 mg, 0.09 mmol, 1.5 eq.), and K₂CO₃ (16 mg, 0.12 mmol, 2.0 eq.) were dissolved in DMF (1.0 mL) and reacted at 50° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, tert-butyl 5-(3-(4-(4-(5-amino-6-(((R)-2-((4-fluorophenyl))amino)-2-oxo-1-phenylethoxy)carbonyl)pyrazin-2-yl)-1H-pyrazol-1-yl)piperidin-1-yl)propyl)hexahydropyrrolo[3,4-c]pyrrole-2(1H)-carboxylate (39 mg, 85%).

¹H NMR (400 MHz, CDCl₃) δ 9.03 (s, 1H), 8.54 (s, 1H), 7.99 (s, 1H), 7.95 (s, 1H), 7.70-7.63 (m, 2H), 7.63-7.54 (m, 2H), 7.49-7.39 (m, 3H), 7.11-6.98 (m, 2H), 6.45 (s, 1H), 6.34 (s, 2H), 4.29-4.18 (m, 1H), 3.60-3.49 (m, 3H), 3.33-3.21 (m, 2H), 3.16-3.06 (m, 2H), 2.88-2.74 (m, 4H), 2.54-2.44 (m, 4H), 2.44-2.36 (m, 2H), 2.26-2.00 (m, 7H), 1.48 (s, 9H).

### (R)-2-((4-fluorophenyl)amino)-2-oxo-1-phenylethyl 3-amino-6-(1-(1-(3-(hexahydropyrrolo[3,4-c]pyrrol-2(1H)-yl)propyl)piperidin-4-yl)-1H-pyrazol-4-yl)pyrazine-2-carboxylate hydrochloride (step 2)

Tert-butyl 5-(3-(4-(4-(5-amino-6-(((R)-2-((4-fluorophenyl)amino)-2-oxo-1-phenylethoxy)carbonyl)pyrazin-2-yl)-1H-pyrazol-1-yl)piperidin-1-yl)propyl)hexahydropyrrolo[3,4-c]pyrrole-2(1H)-carboxylate (39 mg, 0.051 mmol) was dissolved in DCM (1.0 mL). Then, 4 N HCl in dioxane (0.5 mL) was slowly added, and reacted at room temperature for 1 hour. After completion of the reaction, the precipitate was filtered to obtain (R)-2-((4-fluorophenyl)amino)-2-oxo-1-phenylethyl 3-amino-6-(1-(1-(3-(hexahydropyrrolo[3,4-c]pyrrol-2(1H)-yl)propyl)piperidin-4-yl)-1H-pyrazol-4-yl)pyrazine-2-carboxylate hydrochloride (35 mg, mixture).

### Synthesis of (R)-2-((4-fluorophenyl)amino)-2-oxo-1-phenylethyl 3-amino-6-(1-(1-(3-(5-(2-((2-(2,6-dioxopiperidin-3-yl)-1,3-dioxoisoindolin-5-yl)oxy)acetyl)hexahydropyrrolo[3,4-c]pyrrol-2(1H)-yl)propyl)piperidin-4-yl)-1H-pyrazol-4-yl)pyrazine-2-carboxylate [step 3, TDH-466]

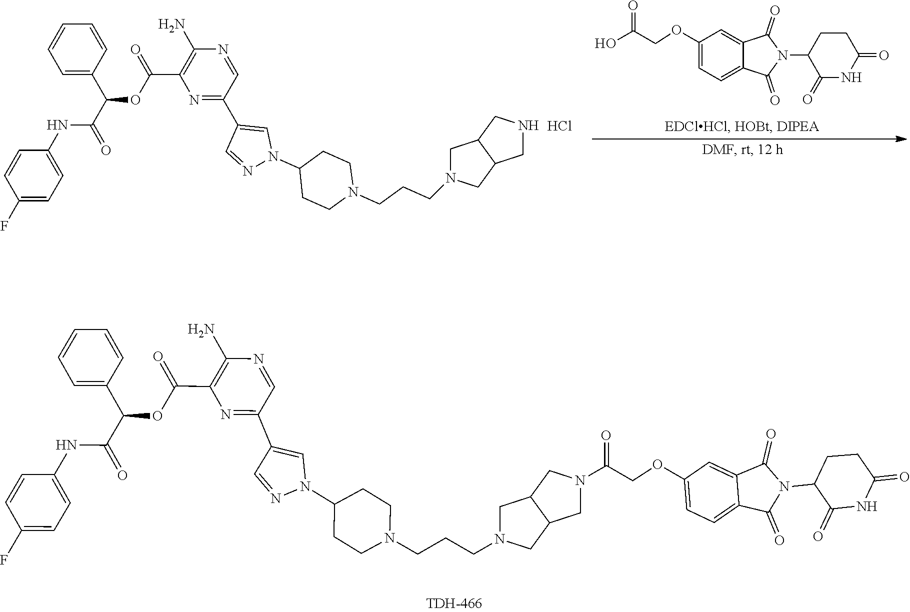 [SMILES: Nc1ncc(-c2cnn(C3CCN(CCCN4CC5CNCC5C4)CC3)c2)nc1C(=O)O[C@@H](C(=O)Nc1ccc(F)cc1)c1ccccc1 ; Cl ; O=C(O)COc1ccc2c(c1)C(=O)N(C1CCC(=O)NC1=O)C2=O ; Nc1ncc(-c2cnn(C3CCN(CCCN4CC5CN(C(=O)COc6ccc7c(c6)C(=O)N(C6CCC(=O)NC6=O)C7=O)CC5C4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(hexahydropyrrolo[3,4-c]pyrrol-2(1H)-yl)propyl)piperidin-4-yl)-1H-pyrazol-4-yl)pyrazine-2-carboxylate hydrochloride (15 mg, 0.021 mmol, 1 eq.), 2-((2-(2,6-dioxopiperidin-3-yl)-1,3-dioxoisoindolin-5-yl)oxy)acetic acid (7 mg, 0.021 mmol, 1.0 eq.), EDCI-HCl (5 mg, 0.023 mmol, 1.1 eq.), HOBt (4 mg, 0.023 mmol, 1.1 eq.) and DIPEA (0.02 mL, 0.11 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(5-(2-((2-(2,6-dioxopiperidin-3-yl)-1,3-dioxoisoindolin-5-yl)oxy)acetyl)hexahydropyrrolo[3,4-c]pyrrol-2(1H)-yl)propyl)piperidin-4-yl)-1H-pyrazol-4-yl)pyrazine-2-carboxylate (14 mg, 68%).

### Synthesis of (R)-2-((4-fluorophenyl)amino)-2-oxo-1-phenylethyl 3-amino-6-(1-(1-(3-(5-((2-(2,6-dioxopiperidin-3-yl)-1,3-dioxoisoindolin-5-yl)glycyl)hexahydropyrrolo[3,4-c]pyrrol-2(1H)-yl)propyl)piperidin-4-yl)-1H-pyrazol-4-yl)pyrazine-2-carboxylate [TDH-467]

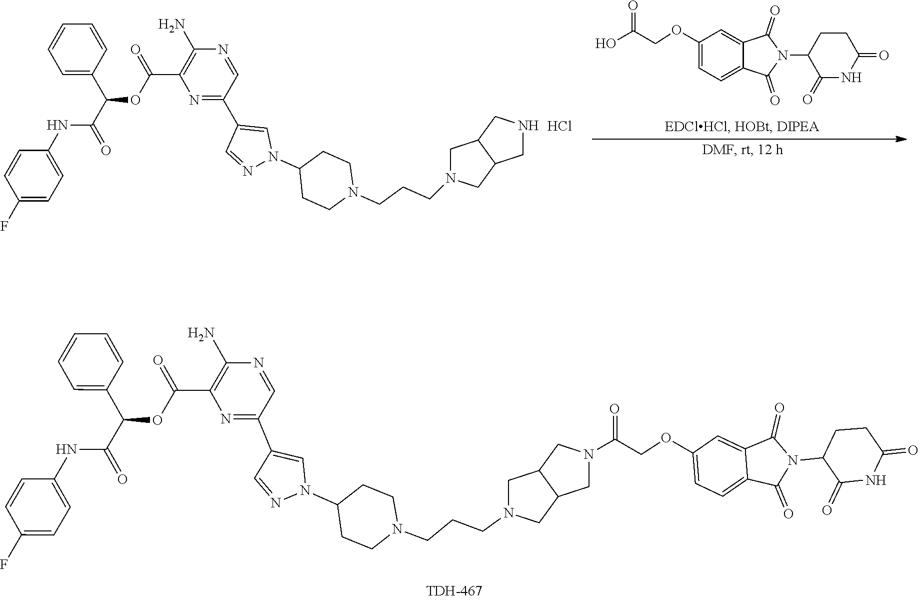 [SMILES: Nc1ncc(-c2cnn(C3CCN(CCCN4CC5CNCC5C4)CC3)c2)nc1C(=O)O[C@@H](C(=O)Nc1ccc(F)cc1)c1ccccc1 ; Cl ; O=C(O)COc1ccc2c(c1)C(=O)N(C1CCC(=O)NC1=O)C2=O ; Nc1ncc(-c2cnn(C3CCN(CCCN4CC5CN(C(=O)COc6ccc7c(c6)C(=O)N(C6CCC(=O)NC6=O)C7=O)CC5C4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(3-(hexahydropyrrolo[3,4-c]pyrrol-2(1H)-yl)propyl)piperidin-4-yl)-1H-pyrazol-4-yl)pyrazine-2-carboxylate hydrochloride (15 mg, 0.021 mmol, 1 eq.), (2-(2,6-dioxopiperidin-3-yl)-1,3-dioxoisoindolin-5-yl)glycine (7 mg, 0.021 mmol, 1.0 eq.), EDCI-HCl (5 mg, 0.023 mmol, 1.1 eq.), HOBt (4 mg, 0.023 mmol), 1.1 eq.), and DIPEA (0.02 mL, 0.11 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(3-(5-((2-(2,6-dioxopiperidin-3-yl)-1,3-dioxoisoindolin-5-yl)glycyl)hexahydropyrrolo[3,4-c]pyrrol-2(1H)-yl)propyl)piperidin-4-yl)-1H-pyrazol-4-yl)pyrazine-2-carboxylate (11 mg, 53%).

### Synthesis of (R)-2-((4-fluorophenyl)amino)-2-oxo-1-phenylethyl 3-amino-6-(1-(1-(3-(5-(3-(2-(2,6-dioxopiperidin-3-yl)-1-methylene-3-oxoisoindolin-5-yl)propanoyl)hexahydropyrrolo[3,4-c]pyrrol-2(1H)-yl)propyl)piperidin-4-yl)-1H-pyrazol-4-yl)pyrazine-2-carboxylate [TDH-480]

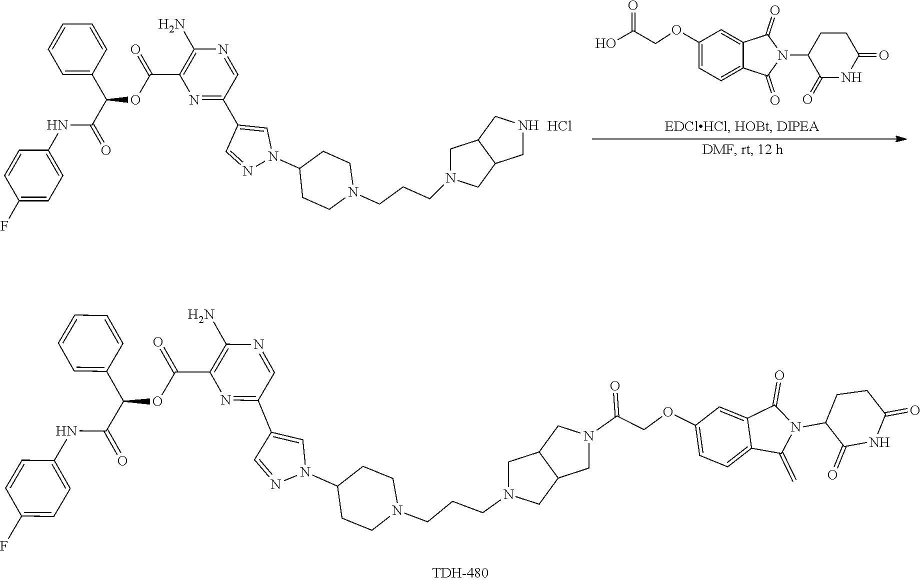 [SMILES: Nc1ncc(-c2cnn(C3CCN(CCCN4CC5CNCC5C4)CC3)c2)nc1C(=O)O[C@@H](C(=O)Nc1ccc(F)cc1)c1ccccc1 ; Cl ; O=C(O)COc1ccc2c(c1)C(=O)N(C1CCC(=O)NC1=O)C2=O ; C=C1c2ccc(OCC(=O)N3CC4CN(CCCN5CCC(n6cc(-c7cnc(N)c(C(=O)O[C@@H](C(=O)Nc8ccc(F)cc8)c8ccccc8)n7)cn6)CC5)CC4C3)cc2C(=O)N1C1CCC(=O)NC1=O]

(R)-2-((4-fluorophenyl)amino)-2-oxo-1-phenylethyl 3-amino-6-(1-(1-(3-(hexahydropyrrolo[3,4-c]pyrrol-2(1H)-yl)propyl)piperidin-4-yl)-1H-pyrazol-4-yl)pyrazine-2-carboxylate hydrochloride (4 mg, 0.006 mmol, 1 eq.), 3-(2-(2,6-dioxopiperidin-3-yl)-1,3-dioxoisoindolin-5-yl)propanoic acid (2 mg, 0.006 mmol, 1.0 eq.), EDCI-HCl (2 mg, 0.007 mmol, 1.1 eq.), HOBt (1 mg, 0.007 mmol, 1.1 eq.) and DIPEA (0.005 mL, 0.03 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, ((R)-2-((4-fluorophenyl)amino)-2-oxo-1-phenylethyl 3-amino-6-(1-(1-(3-(5-(3-(2-(2,6-dioxopiperidin-3-yl)-1-methylene-3-oxoisoindolin-5-yl)propanoyl)hexahydropyrrolo[3,4-c]pyrrol-2 (1H)-yl)propyl)piperidin-4-yl)-1H-pyrazol-4-yl)pyrazine-2-carboxylate (3 mg, 53%).

### Synthesis of (R)-2-((4-fluorophenyl)amino)-2-oxo-1-phenylethyl 3-amino-6-(1-(1-(((1r,4r)-4-((3-(2,4-dioxotetrahydropyrimidin-1(2H)-yl)-4-methoxybenzamido)methyl)cyclohexyl)methyl)piperidin-4-yl)-1H-pyrazol-4-yl)pyrazine-2-carboxylate [TDH-497]

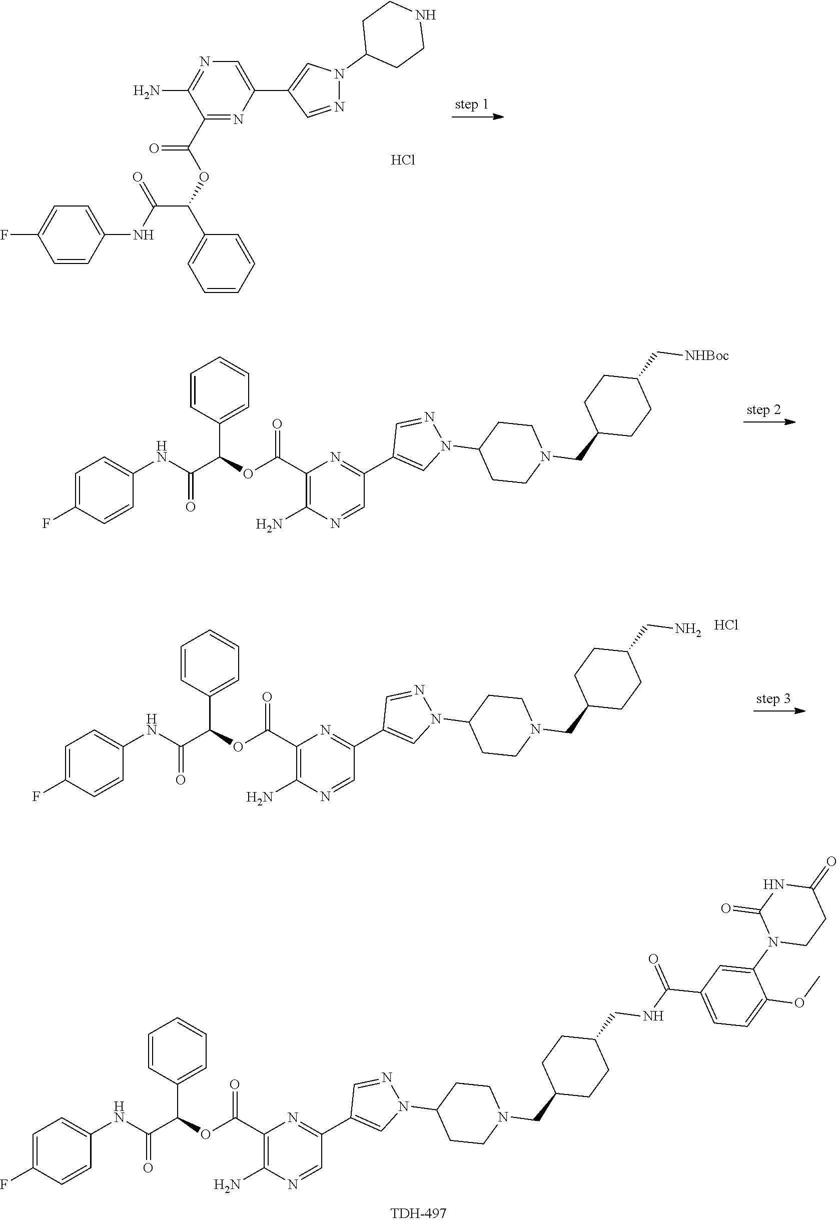 [SMILES: Nc1ncc(-c2cnn(C3CCNCC3)c2)nc1C(=O)O[C@@H](C(=O)Nc1ccc(F)cc1)c1ccccc1 ; Cl ; CC(C)(C)OC(=O)NC[C@H]1CC[C@H](CN2CCC(n3cc(-c4cnc(N)c(C(=O)O[C@@H](C(=O)Nc5ccc(F)cc5)c5ccccc5)n4)cn3)CC2)CC1 ; Cl ; NC[C@H]1CC[C@H](CN2CCC(n3cc(-c4cnc(N)c(C(=O)O[C@@H](C(=O)Nc5ccc(F)cc5)c5ccccc5)n4)cn3)CC2)CC1 ; COc1ccc(C(=O)NC[C@H]2CC[C@H](CN3CCC(n4cc(-c5cnc(N)c(C(=O)O[C@@H](C(=O)Nc6ccc(F)cc6)c6ccccc6)n5)cn4)CC3)CC2)cc1N1CCC(=O)NC1=O]

### (R)-2-((4-fluorophenyl)amino)-2-oxo-1-phenylethyl 3-amino-6-(1-(1-(((1 r,4r)-4-(((tert-butoxycarbonyl)amino)methyl)cyclohexyl)methyl)piperidin-4-yl)-1H-pyrazol-4-yl)pyrazine-2-carboxylate (step 1)

(R)-2-((4-fluorophenyl)amino)-2-oxo-1-phenylethyl 3-amino-6-(1-(piperidin-4-yl)-1H-pyrazol-4-yl)pyrazine-2-carboxylate hydrochloride (300 mg, 0.57 mmol, 1.0 eq.), tert-butyl (((1 r,4r)-4-(iodomethyl)cyclohexyl)methyl)carbamate (305 mg, 0.86 mmol, 1.5 eq.), and K₂CO₃ (158 mg, 1.14 mmol, 2.0 eq.) were dissolved in DMF (2.3 mL) and reacted at 50° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(((1 r,4r)-4-(((tert-butoxycarbonyl)amino)methyl)cyclohexyl)methyl)piperidin-4-yl)-1H-pyrazol-4-yl)pyrazine-2-carboxylate (282 mg, 67%).

¹H NMR (400 MHz, CDCl₃) δ 8.98 (s, 1H), 8.53 (s, 1H), 7.99 (s, 1H), 7.94 (s, 1H), 7.69-7.62 (m, 2H), 7.61-7.54 (m, 2H), 7.48-7.38 (m, 3H), 7.09-7.01 (m, 2H), 6.44 (s, 1H), 6.34 (s, 2H), 4.61 (s, 1H), 4.21 (s, 1H), 3.05-2.97 (m, 4H), 2.23-2.17 (m, 3H), 2.12-2.02 (m, 4H), 1.89-1.84 (m, 2H), 1.84-1.73 (m, 4H), 1.47 (s, 9H), 1.04-0.84 (m, 5H).

### (R)-2-((4-fluorophenyl)amino)-2-oxo-1-phenylethyl 3-amino-6-(1-(1-(((1 r,4r)-4-(aminomethyl)cyclohexyl)methyl)piperidin-4-yl)-1H-pyrazol-4-yl)pyrazine-2-carboxylate hydrochloride (step 2)

(R)-2-((4-fluorophenyl)amino)-2-oxo-1-phenylethyl 3-amino-6-(1-(1-(((1r,4r)-4-(((tert-butoxycarbonyl)amino)methyl)cyclohexyl)methyl)piperidin-4-yl)-1H-pyrazol-4-yl)pyrazine-2-carboxylate (282 mg, 0.38 mmol) was dissolved in DCM (2.0 mL). Then, 4 N HCl in dioxane (1.0 mL) was added slowly and reacted at room temperature for 1 hour. After completion of the reaction, the precipitate was filtered to obtain (R)-2-((4-fluorophenyl)amino)-2-oxo-1-phenylethyl 3-amino-6-(1-(1-(((1 r,4r)-4-(aminomethyl)cyclohexyl)methyl)piperidin-4-yl)-1H-pyrazol-4-yl)pyrazine-2-carboxylate hydrochloride (250 mg, mixture).

### Synthesis of (R)-2-((4-fluorophenyl)amino)-2-oxo-1-phenylethyl 3-amino-6-(1-(1-(((1 r,4r)-4-((3-(2,4-dioxotetrahydropyrimidin-1(2H)-yl)-4-methoxybenzamido)methyl)cyclohexyl)methyl)piperidin-4-yl)-1H-pyrazol-4-yl)pyrazine-2-carboxylate [step 3, TDH-497]

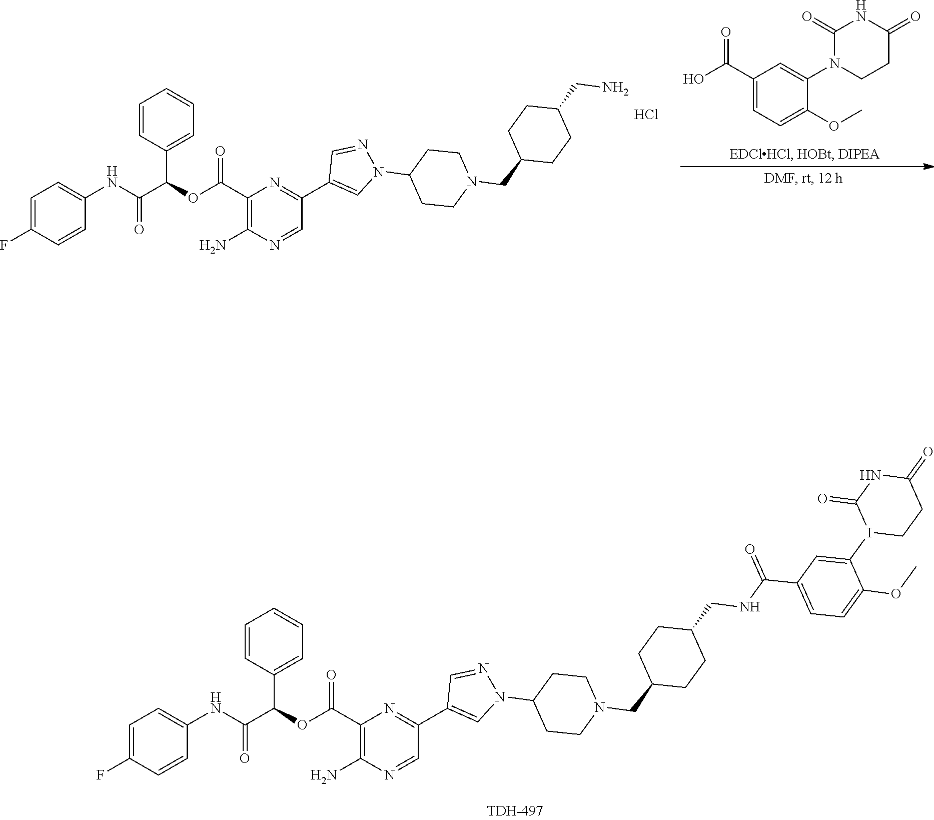 [SMILES: NC[C@H]1CC[C@H](CN2CCC(n3cc(-c4cnc(N)c(C(=O)O[C@@H](C(=O)Nc5ccc(F)cc5)c5ccccc5)n4)cn3)CC2)CC1 ; Cl ; COc1ccc(C(=O)O)cc1N1CCC(=O)NC1=O ; COc1ccc(C(=O)NC[C@H]2CC[C@H](CN3CCC(n4cc(-c5cnc(N)c(C(=O)O[C@@H](C(=O)Nc6ccc(F)cc6)c6ccccc6)n5)cn4)CC3)CC2)cc1I1CCC(=O)NC1=O]

(R)-2-((4-fluorophenyl)amino)-2-oxo-1-phenylethyl 3-amino-6-(1-(1-(((1 r,4r)-4-(aminomethyl)cyclohexyl)methyl)piperidin-4-yl)-1H-pyrazol-4-yl)pyrazine-2-carboxylate hydrochloride (20 mg, 0.030 mmol, 1 eq.), 3-(2,4-dioxotetrahydropyrimidin-1(2H)-yl)-4-methoxybenzoic acid (8 mg, 0.030 mmol, 1.0 eq.), EDCI-HCl (6 mg, 0.032 mmol, 1.1 eq.), HOBt (5 mg, 0.032 mmol, 1.1 eq.), and DIPEA (0.03 mL, 0.15 mmol, 5 eq.) were dissolved in DMF (1.0 mL) and reacted at room temperature overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(((1 r,4r)-4-((3-(2,4-dioxotetrahydropyrimidin-1(2H)-yl)-4-methoxybenzamido)methyl)cyclohexyl)methyl)piperidin-4-yl)-1H-pyrazol-4-yl)pyrazine-2-carboxylate (18 mg, 68%).

### Synthesis of (R)-2-((4-fluorophenyl)amino)-2-oxo-1-phenylethyl 3-amino-6-(1-(1-(((1 r,4r)-4-((2-(3-((1-(2,6-dioxopiperidin-3-yl)-2,5-dioxo-2,5-dihydro-1H-pyrrol-3-yl)amino)phenyl)acetamido)methyl)cyclohexyl)methyl)piperidin-4-yl)-1H-pyrazol-4-yl)pyrazine-2-carboxylate [TDH-498]

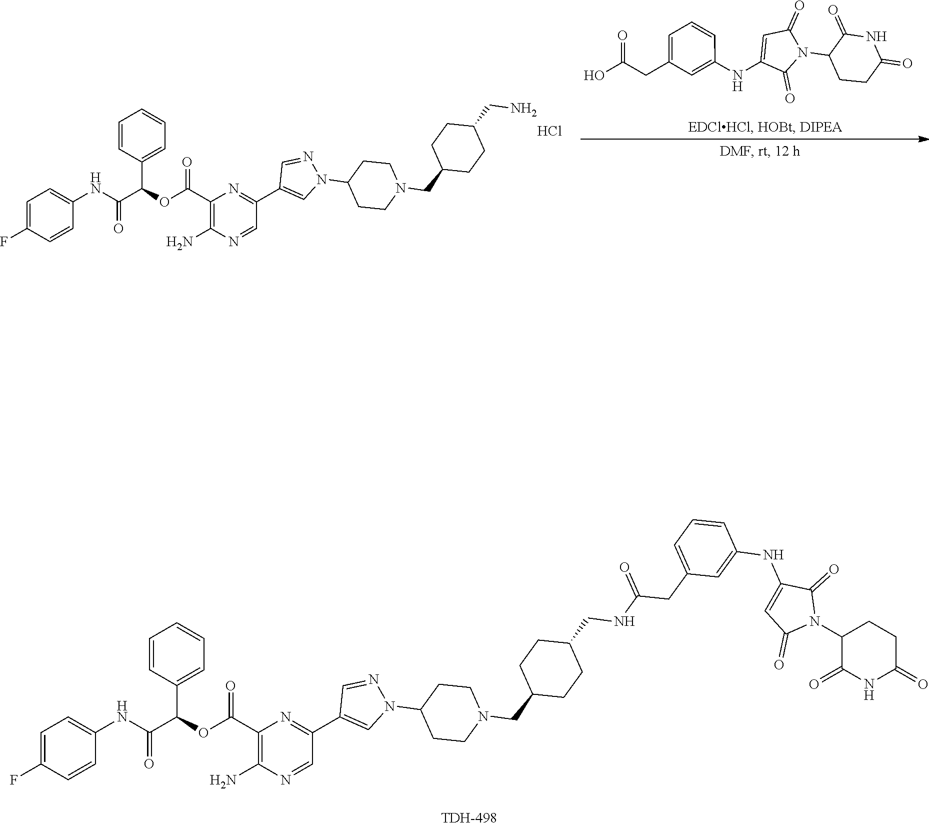 [SMILES: O=C(O)Cc1cccc(NC2=CC(=O)N(C3CCC(=O)NC3=O)C2=O)c1 ; NC[C@H]1CC[C@H](CN2CCC(n3cc(-c4cnc(N)c(C(=O)O[C@@H](C(=O)Nc5ccc(F)cc5)c5ccccc5)n4)cn3)CC2)CC1 ; Cl ; Nc1ncc(-c2cnn(C3CCN(C[C@H]4CC[C@H](CNC(=O)Cc5cccc(NC6=CC(=O)N(C7CCC(=O)NC7=O)C6=O)c5)CC4)CC3)c2)nc1C(=O)O[C@@H](C(=O)Nc1ccc(F)cc1)c1ccccc1]

(R)-2-((4-fluorophenyl)amino)-2-oxo-1-phenylethyl 3-amino-6-(1-(1-(((1 r,4r)-4-(aminomethyl)cyclohexyl)methyl)piperidin-4-yl)-1H-pyrazol-4-yl)pyrazine-2-carboxylate hydrochloride (20 mg, 0.030 mmol, 1 eq.), 2-(3-((1-(2,6-dioxopiperidin-3-yl)-2,5-dioxo-2,5-dihydro-1H-pyrrol-3-yl)amino)phenyl)acetic acid (11 mg, 0.030 mmol, 1.0 eq.), EDCI-HCl (6 mg, 0.032 mmol, 1.1 eq.), HOBt (5 mg, 0.032 mmol, 1.1 eq.), and DIPEA (0.03 mL, 0.15 mmol, 5 eq.) were dissolved in DMF (1.0 mL) and reacted overnight at room temperature. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, (R)-2-((4-fluorophenyl)amino)-2-oxo-1-phenylethyl 3-amino-6-(1-(1-(((1 r,4r)-4-((2-(3-((1-(2,6-dioxopiperidin-3-yl)-2,5-dioxo-2,5-dihydro-1H-pyrrol-3-yl)amino)phenyl)acetamido)methyl)cyclohexyl)methyl)piperidin-4-yl)-1H-pyrazol-4-yl)pyrazine-2-carboxylate (14 mg, 49%).

The E3 ligase binder moieties used in the previous synthesis methods were prepared through the following methods.

### Synthesis of 1-(1-(2,6-dioxopiperidin-3-yl)-2,5-dioxo-2,5-dihydro-1H-pyrrol-3-yl)piperidine-4-carboxylic acid

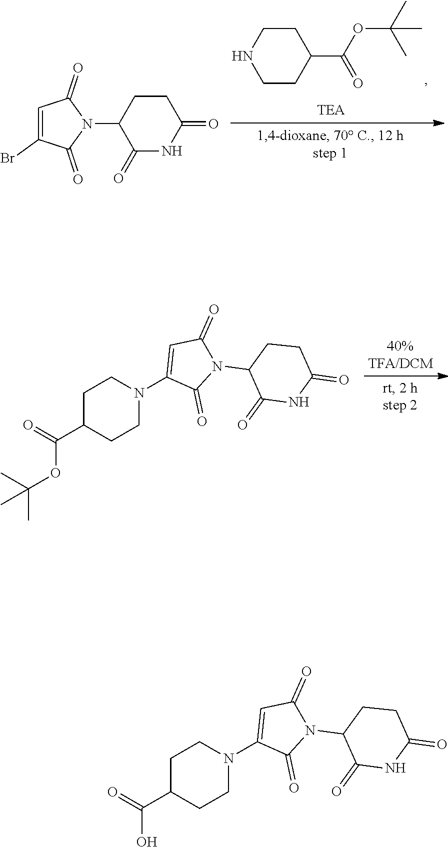 [SMILES: O=C1CCC(N2C(=O)C=C(Br)C2=O)C(=O)N1 ; CC(C)(C)OC(=O)C1CCNCC1 ; CC(C)(C)OC(=O)C1CCN(C2=CC(=O)N(C3CCC(=O)NC3=O)C2=O)CC1 ; O=C1CCC(N2C(=O)C=C(N3CCC(C(=O)O)CC3)C2=O)C(=O)N1]

### Tert-butyl 1-(1-(2,6-dioxopiperidin-3-yl)-2,5-dioxo-2,5-dihydro-1H-pyrrol-3-yl)piperidine-4-carboxylate (step 1)

3-(3-bromo-2,5-dioxo-2,5-dihydro-1H-pyrrol-1-yl)piperidine-2,6-dione (20 mg, 0.07 mol, 1.0 eq.), tert-butyl piperidine-4-carboxylate (0.02 mL, 0.11 mmol, 1.5 eq.) and TEA (0.03 mL, 0.21 mmol, 3 eq.) were dissolved in 1,4-dioxane (1.0 mL), and the mixture was reacted at 70° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, tert-butyl 1-(1-(2,6-dioxopiperidin-3-yl)-2,5-dioxo-2,5-dihydro-1H-pyrrol-3-yl)piperidine-4-carboxylate (26 mg, 95%).

¹H NMR (300 MHz, DMSO) δ 11.00 (s, 1H), 5.26 (s, 1H), 4.92-4.81 (m, 1H), 3.26-3.12 (m, 2H), 2.90-2.77 (m, 1H), 2.62-2.52 (m, 3H), 2.44-2.33 (m, 1H), 1.96-1.83 (m, 3H), 1.67-1.52 (m, 2H), 1.49-1.34 (m, 9H).

### 1-(1-(2,6-dioxopiperidin-3-yl)-2,5-dioxo-2,5-dihydro-1H-pyrrol-3-yl)piperidine-4-carboxylic acid (step 2)

Tert-butyl 1-(1-(2,6-dioxopiperidin-3-yl)-2,5-dioxo-2,5-dihydro-1H-pyrrol-3-yl)piperidine-4-carboxylate (396 mg, 1.0 mmol) was dissolved in DCM (5.0 mL). Then, TFA (2.0 mL) was added, and reacted at room temperature for 2 hours. The reaction solvent was concentrated to obtain a yellow solid, 1-(1-(2,6-dioxopiperidin-3-yl)-2,5-dioxo-2,5-dihydro-1H-pyrrol-3-yl)piperidine-4-carboxylic acid (333 mg, quant.).

¹H NMR (300 MHz, DMSO) δ 12.41 (s, 1H), 11.08-10.91 (m, 1H), 5.26 (s, 1H), 4.89-4.81 (m, 1H), 3.28-3.14 (m, 2H), 3.00-2.72 (m, 2H), 2.65-2.54 (m, 1H), 2.48-2.35 (m, 2H), 2.02-1.84 (m, 4H), 1.71-1.57 (m, 2H).

### Synthesis of 1-(1-(2,6-dioxopiperidin-3-yl)-2,5-dioxo-2,5-dihydro-1H-pyrrol-3-yl)azetidine-3-carboxylic acid

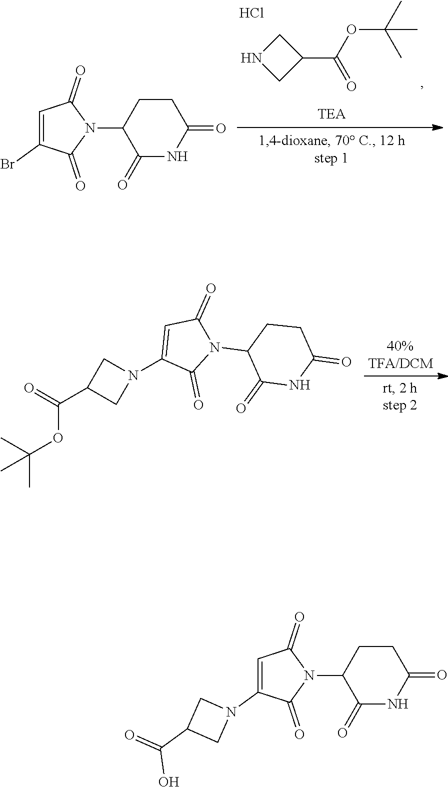 [SMILES: O=C1CCC(N2C(=O)C=C(Br)C2=O)C(=O)N1 ; Cl ; CC(C)(C)OC(=O)C1CNC1 ; CC(C)(C)OC(=O)C1CN(C2=CC(=O)N(C3CCC(=O)NC3=O)C2=O)C1 ; O=C1CCC(N2C(=O)C=C(N3CC(C(=O)O)C3)C2=O)C(=O)N1]

### Tert-butyl 1-(1-(2,6-dioxopiperidin-3-yl)-2,5-dioxo-2,5-dihydro-1H-pyrrol-3-yl)azetidine-3-carboxylate (step 1)

3-(3-bromo-2,5-dioxo-2,5-dihydro-1H-pyrrol-1-yl)piperidine-2,6-dione (400 mg, 1.4 mol, 1.0 eq.), tert-butyl azetidine-3-carboxylate hydrochloride (405 mg, 2.1 mmol, 1.5 eq.) and TEA (1.2 mL, 8.4 mmol, 6 eq.) were dissolved in 1,4-dioxane (5.6 mL), and the mixture was reacted at 70° C. overnight. After completion of the reaction, it was diluted with water and extracted with EA. The combined organic layer was washed with brine, and the residual water was removed through anhydrous Na₂SO₄, followed by filtration. The organic layer was concentrated under reduced pressure. The concentrate was separated and purified by silica gel column chromatography to obtain the desired yellow solid, tert-butyl 1-(1-(2,6-dioxopiperidin-3-yl)-2,5-dioxo-2,5-dihydro-1H— This gave pyrrol-3-yl)azetidine-3-carboxylate (413 mg, 81%).

¹H NMR (400 MHz, DMSO) δ 10.99 (s, 1H), 4.94 (s, 1H), 4.87-4.77 (m, 1H), 4.57-3.95 (m, 4H), 3.73-3.61 (m, 1H), 2.91-2.76 (m, 1H), 2.60-2.54 (m, 1H), 2.46-2.32 (m, 1H), 1.96-1.85 (m, 1H), 1.44 (s, 9H).

### 1-(1-(2,6-dioxopiperidin-3-yl)-2,5-dioxo-2,5-dihydro-1H-pyrrol-3-yl)azetidine-3-carboxylic acid (step 2)

Tert-butyl 1-(1-(2,6-dioxopiperidin-3-yl)-2,5-dioxo-2,5-dihydro-1H-pyrrol-3-yl)azetidine-3-carboxylate (100 mg, 0.27 mmol) was dissolved in DCM (2.0 mL). Then, TFA (0.5 mL) was added, and reacted at room temperature for 2 hours. The reaction solvent was concentrated to obtain a yellow solid, 1-(1-(2,6-dioxopiperidin-3-yl)-2,5-dioxo-2,5-dihydro-1H-pyrrol-3-yl)azetidine-3-carboxylic acid (109 mg, mixture).

### Synthesis of 3-(2-(2,6-dioxopiperidin-3-yl)-1-oxoisoindolin-5-yl)propanoic acid

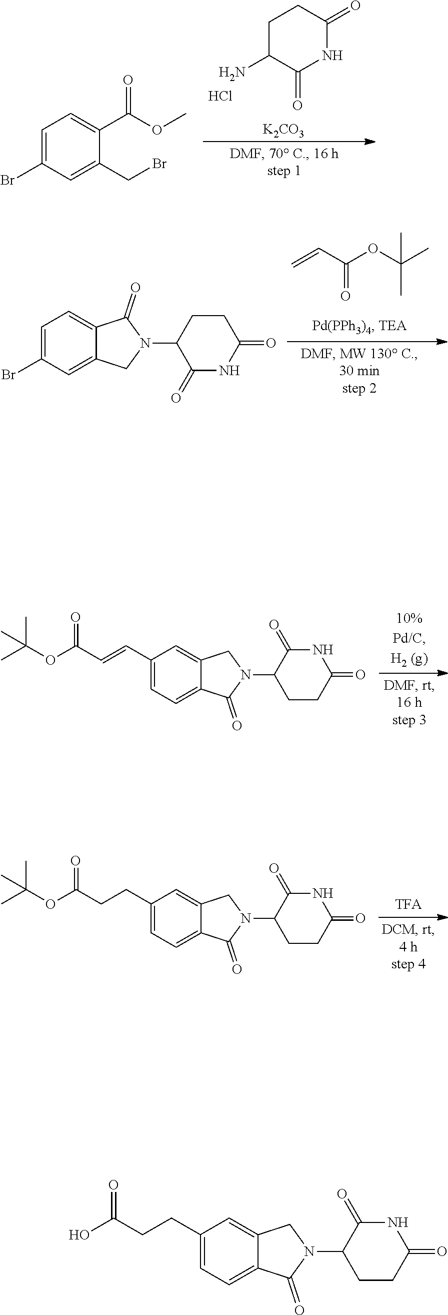

### 3-(5-bromo-1-oxoisoindolin-2-yl)piperidine-2,6-dione (step 1)

Methyl 4-bromo-2-(bromomethyl)benzoate (850 mg, 2.8 mmol, 1.0 eq.), 3-aminopiperidine-2,6-dione hydrochloride (500 mg, 3.0 mmol, 1.1 eq.), and K₂CO₃ (1.14 g, 0.94 mmol, 3 eq.) were dissolved in DMF (11 mL) and reacted at 70° C. overnight. After completion of the reaction, the temperature was lowered to room temperature, water was added, and the mixture was stirred for 30 minutes. Then, the mixture was filtered with water to obtain the desired pale yellow solid, 3-(5-bromo-1-oxoisoindolin-2-yl)piperidine-2,6-dione (589 mg, 65%).

¹H NMR (400 MHz, DMSO) δ 11.00 (s, 1H), 7.90 (d, J=1.6 Hz, 1H), 7.78-7.61 (m, 2H), 5.17-5.04 (m, 1H), 4.48 (d, J=17.7 Hz, 1H), 4.35 (d, J=17.7 Hz, 1H), 2.97-2.84 (m, 1H), 2.65-2.56 (m, 1H), 2.44-2.33 (m, 1H), 2.06-1.95 (m, 1H).

### Tert-butyl (E)-3-(2-(2,6-dioxopiperidin-3-yl)-1-oxoisoindolin-5-yl)acrylate (step 2)

3-(5-bromo-1-oxoisoindolin-2-yl)piperidine-2,6-dione (50 mg, 0.15 mmol, 1.0 eq.), tert-butyl acrylate (0.048 mL, 0.32 mmol, 2.1 eq.), Pd(PPh₃)₄ (25 mg, 0.022 mmol, 0.14 eq.) and TEA (0.02 mL, 0.15 mmol, 1.0 eq.) were dissolved in DMF (1 mL), and reacted in a microwave reactor at 130° C. for 30 minutes. After cooling to room temperature, the mixture was filtered through celite. After concentrating the solution, the concentrate was separated and purified by silica gel column chromatography to obtain the desired yellow solid, tert-butyl (E)-3-(2-(2,6-dioxopiperidin-3-yl)-1-oxoisoindolin-5-yl)acrylate (62 mg, mixture).

### Tert-butyl 3-(2-(2,6-dioxopiperidin-3-yl)-1-oxoisoindolin-5-yl)propanoate (step 3)

Tert-butyl (E)-3-(2-(2,6-dioxopiperidin-3-yl)-1-oxoisoindolin-5-yl)acrylate (62 mg, 1.7 mmol, 1.0 eq.) was dissolved in DMF (7 mL). Then, 10% Pd/C (200 mg) was added and H₂ (g) balloon was inserted thereto. The mixture was reacted for 16 hours at room temperature. After completion of the reaction, the mixture was filtered through celite. The solution was concentrated to obtain tert-butyl 3-(2-(2,6-dioxopiperidin-3-yl)-1-oxoisoindolin-5-yl)propanoate (38 mg, mixture) as a black solid.

### 3-(2-(2,6-dioxopiperidin-3-yl)-1-oxoisoindolin-5-yl)propanoic acid (step 4)

Tert-butyl 3-(2-(2,6-dioxopiperidin-3-yl)-1-oxoisoindolin-5-yl)propanoate (38 mg, 0.10 mmol, 1.0 eq.) was dissolved in DCM (1.5 mL). Then, TFA (0.7 mL) was added and reacted at room temperature for 2 hours. The reaction solvent was concentrated to obtain 3-(2-(2,6-dioxopiperidin-3-yl)-1-oxoisoindolin-5-yl)propanoic acid (23 mg, 73%) as a white solid.

¹H NMR (400 MHz, DMSO) δ 10.98 (s, 1H), 7.64 (d, J=7.8 Hz, 1H), 7.47 (s, 1H), 7.39 (d, J=7.8 Hz, 1H), 5.10 (dd, J=13.3, 5.1 Hz, 1H), 4.42 (d, J=17.3 Hz, 1H), 4.29 (d, J=17.2 Hz, 1H), 2.99-2.89 (m, 3H), 2.66-2.56 (m, 3H), 2.44-2.34 (m, 1H), 2.05-1.97 (m, 1H).

### Synthesis of 2-(2,6-dioxopiperidin-3-yl)-1-oxoisoindoline-5-carboxylic acid

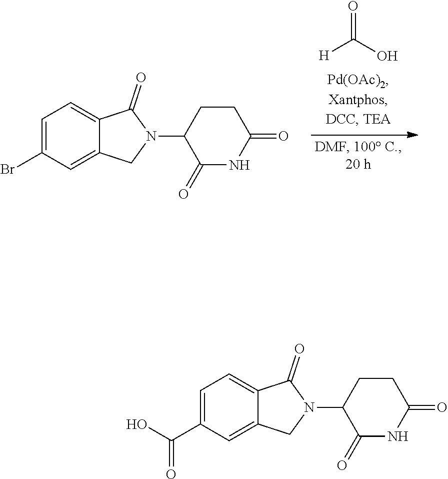 [SMILES: O=CO ; O=C1CCC(N2Cc3cc(Br)ccc3C2=O)C(=O)N1 ; O=C1CCC(N2Cc3cc(C(=O)O)ccc3C2=O)C(=O)N1]

3-(5-bromo-1-oxoisoindolin-2-yl)piperidine-2,6-dione (300 mg, 0.93 mmmol, 1.0 eq.), formic acid (0.5 mL, 65 mmmol, 7.0 eq.), Pd(OAc)₂ (6 mg, 0.028 mmol, 0.03 eq.), Xantphos (316 mg, 0.028 mmol, 0.03 eq.), DCC (38 mg, 0.19 mmmol, 0.2 eq.), and TEA (0.26 mL, 1.9 mmmol, 2.0 eq.) were dissolved in DMF (3.7 mL) and reacted at 100° C. for 20 hours. After completion of the reaction, it was diluted with water and extracted with EA. The combined organic layer was washed with brine, and the residual water was removed through anhydrous Na₂SO₄, followed by filtration. The organic layer was concentrated under reduced pressure and purified by silica gel column chromatography to obtain 2-(2,6-dioxopiperidin-3-yl)-1-oxoisoindoline-5-carboxylic acid (110 mg, %) as a light brown solid.

¹H NMR (300 MHz, DMSO) δ 11.03 (s, 1H), 8.18 (s, 1H), 8.08 (d, J=8.0 Hz, 1H), 7.83 (d, J=7.9 Hz, 1H), 5.21-5.10 (m, 1H), 4.55 (d, J=17.7 Hz, 1H), 4.41 (d, J=17.7 Hz, 1H), 3.01-2.84 (m, 1H), 2.68-2.56 (m, 1H), 2.45-2.32 (m, 1H), 2.10-1.97 (m, 1H).

### Synthesis of 1-(1-(2,6-dioxopiperidin-3-yl)-2,5-dioxo-2,5-dihydro-1H-pyrrol-3-yl)azetidine-3-carboxylic acid

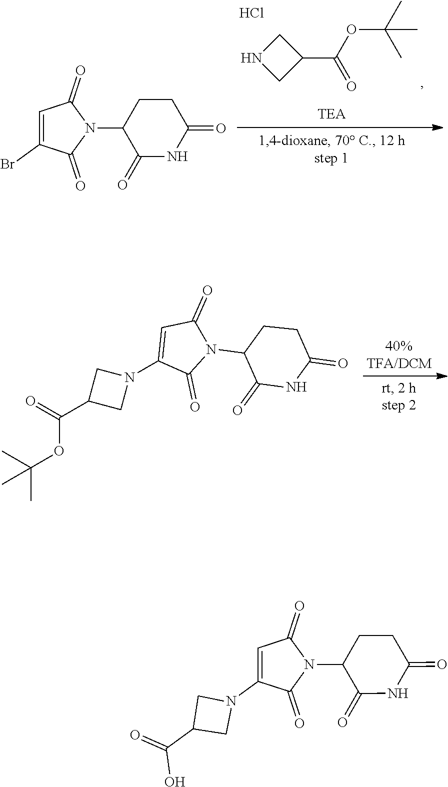 [SMILES: O=C1CCC(N2C(=O)C=C(Br)C2=O)C(=O)N1 ; Cl ; CC(C)(C)OC(=O)C1CNC1 ; CC(C)(C)OC(=O)C1CN(C2=CC(=O)N(C3CCC(=O)NC3=O)C2=O)C1 ; O=C1CCC(N2C(=O)C=C(N3CC(C(=O)O)C3)C2=O)C(=O)N1]

### Tert-butyl 1-(1-(2,6-dioxopiperidin-3-yl)-2,5-dioxo-2,5-dihydro-1H-pyrrol-3-yl)azetidine-3-carboxylate (step 1)

3-(3-bromo-2,5-dioxo-2,5-dihydro-1H-pyrrol-1-yl)piperidine-2,6-dione (400 mg, 1.4 mmol, 1.0 eq.), tert-butyl azetidine After dissolving-3-carboxylate hydrochloride (405 mg, 2.1 mmol, 1.5 eq.) and TEA (1.2 mL, 8.4 mmol, 6 eq.) were dissolved in 1,4-dioxane (5.6 mL), and the mixture was reacted at 70° C. overnight. After completion of the reaction, it was diluted with water and extracted with EA. The combined organic layer was filtered after removing the residual water through anhydrous Na₂SO₄. The organic layer was concentrated under reduced pressure to obtain the desired yellow solid, tert-butyl 1-(1-(2,6-dioxopiperidin-3-yl)-2,5-dioxo-2,5-dihydro-1H-pyrrol-3-yl)azetidine-3-carboxylate (413 mg, 81%).

¹H NMR (400 MHz, DMSO) δ 10.99 (s, 1H), 4.94 (s, 1H), 4.87-4.77 (m, 1H), 4.57-3.95 (m, 4H), 3.73-3.61 (m, 1H), 2.91-2.76 (m, 1H), 2.60-2.54 (m, 1H), 2.46-2.32 (m, 1H), 1.96-1.85 (m, 1H), 1.44 (s, 9H).

### 1-(1-(2,6-dioxopiperidin-3-yl)-2,5-dioxo-2,5-dihydro-1H-pyrrol-3-yl)azetidine-3-carboxylic acid (step 2)

Tert-butyl 1-(1-(2,6-dioxopiperidin-3-yl)-2,5-dioxo-2,5-dihydro-1H-pyrrol-3-yl)azetidine-3-carboxylate (100 mg, 0.27 mmol, 1.0 eq.) was dissolved in DCM (2.0 mL), and TFA (0.5 mL) was added thereto, followed by reaction at room temperature for 2 hours. The reaction solvent was concentrated to obtain a white solid, 1-(1-(2,6-dioxopiperidin-3-yl)-2,5-dioxo-2,5-dihydro-1H-pyrrol-3-yl)azetidine-3-carboxylic acid (109 mg, mixture).

### Synthesis of 4-((2,6-dioxopiperidin-3-yl)carbamoyl)benzoic acid

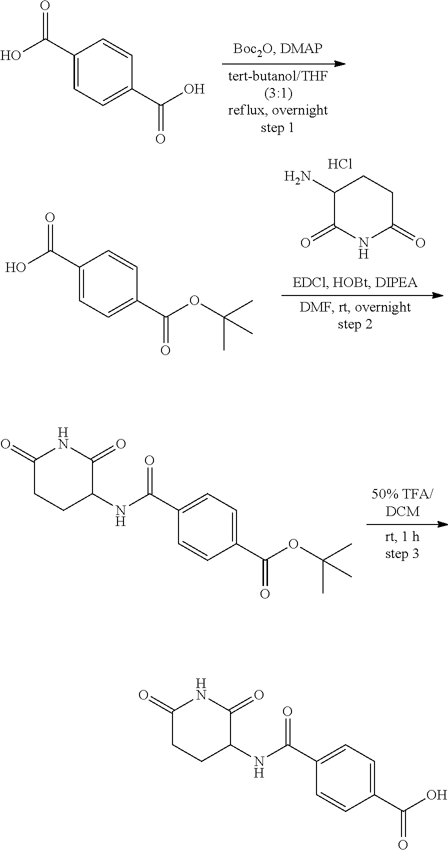 [SMILES: O=C(O)c1ccc(C(=O)O)cc1 ; Cl ; CC(C)(C)OC(=O)c1ccc(C(=O)O)cc1 ; NC1CCC(=O)NC1=O ; CC(C)(C)OC(=O)c1ccc(C(=O)NC2CCC(=O)NC2=O)cc1 ; O=C1CCC(NC(=O)c2ccc(C(=O)O)cc2)C(=O)N1]

### Synthesis of 4-(tert-butoxycarbonyl)benzoic acid (step 1)

Terephthalic acid (2 g, 12.10 mmol, 2 eq) was dissolved in tert-butanol/THF (3:1, 20 mL). Then, di-tert-butyl dicarbonate (1.389 mL, 1.2 eq) and DMAP (348 mg, 0.3 eq) were added thereto and stirred for 16 hours under a reflux condition. The reactant was concentrated, diluted with EtOAc, washed with saturated NaCl aqueous solution, dried over anhydrous MgSO₄, and filtered. The collected organic solvents were concentrated under reduced pressure and purified by silica gel column chromatography to obtain the desired white solid, 4-(tert-butoxycarbonyl)benzoic acid (330 mg, 1.485 mmol, 24%).

¹H NMR (500 MHz, DMSO-d₆) δ 8.04 (d, J=8.5 Hz, 2H), 8.00 (d, J=8.5 Hz, 2H), 1.57 (s, 9H).

### Synthesis of tert-butyl 4-((2,6-dioxopiperidin-3-yl)carbamoyl)benzoate (step 2)

3-aminopiperidine-2,6-dione hydrochloride (40.7 mg, 0.247 mmol, 1.1 eq), 4-(tert-butoxycarbonyl)benzoic acid (50 mg, 1 eq), EDCI-HCl (43.1 mg, 1 eq), and HOBt (34.5 mg, 1 eq) were dissolved in DMF (1 mL), and after adding DIPEA (98 μL, 2.5 eq), the mixture was stirred at room temperature for 16 hours. The reactant was concentrated, diluted with EtOAc, washed with saturated NaCl aqueous solution, dried over anhydrous MgSO₄, and filtered. The filtrate was concentrated under reduced pressure and purified by silica gel column chromatography to obtain the desired white solid, tert-butyl 4-((2,6-dioxopiperidin-3-yl)carbamoyl)benzoate (35 mg, 0.105 mmol, 46%).

¹H NMR (400 MHz, DMSO-d₆) δ 10.89 (s, 1H), 8.96 (d, J=8.3 Hz, 1H), 8.01 (d, J=8.6 Hz, 2H), 7.97 (d, J=8.4 Hz, 2H), 4.86-4.76 (m, 1H), 2.89-2.75 (m, 1H), 2.60-2.56 (m, 1H), 2.19-2.10 (m, 1H), 2.05-1.95 (m, 1H), 1.57 (s, 9H).

### Synthesis of 4-((2,6-dioxopiperidin-3-yl)carbamoyl)benzoic acid (step 3)

Tert-butyl 4-((2,6-dioxopiperidin-3-yl)carbamoyl)benzoate (20 mg, 0.060 mmol, 1 eq.) was dissolved in 50% TFA/DCM (2 mL) and the mixture was stirred at room temperature for 1 hour. The reactant was concentrated under reduced pressure to obtain the desired white solid, 4-((2,6-dioxopiperidin-3-yl)carbamoyl)benzoic acid (10 mg, 0.036 mmol, 60%).

¹H NMR (500 MHz, DMSO-d₆) δ 10.91 (s, 1H), 8.96 (d, J=8.4 Hz, 1H), 8.08-8.03 (m, 2H), 8.01-7.95 (m, 2H), 4.85-4.78 (m, 1H), 2.87-2.78 (m, 1H), 2.60-2.55 (m, 1H), 2.18-2.09 (m, 1H), 2.04-1.97 (m, 1H).

### Synthesis of 3-((2,6-dioxopiperidin-3-yl)carbamoyl)benzoic acid

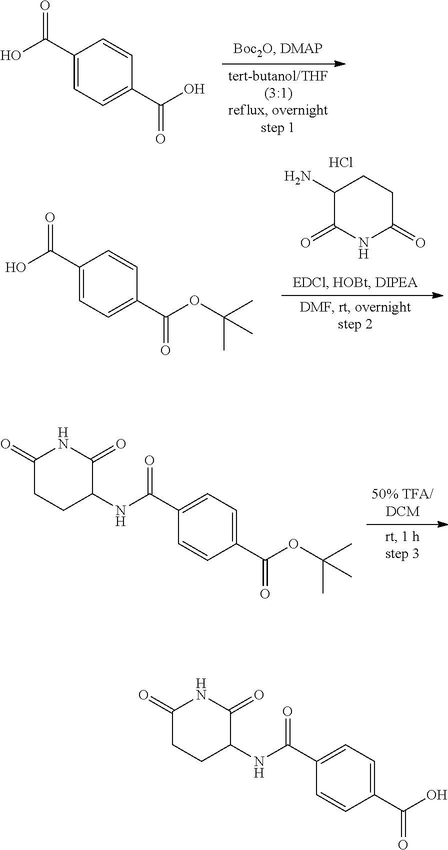 [SMILES: O=C(O)c1ccc(C(=O)O)cc1 ; Cl ; CC(C)(C)OC(=O)c1ccc(C(=O)O)cc1 ; NC1CCC(=O)NC1=O ; CC(C)(C)OC(=O)c1ccc(C(=O)NC2CCC(=O)NC2=O)cc1 ; O=C1CCC(NC(=O)c2ccc(C(=O)O)cc2)C(=O)N1]

### Synthesis of 3-(tert-butoxycarbonyl)benzoic acid (step 1)

Isophthalic acid (152 mg, 0.916 mmol, 2 eq) was dissolved in tert-butanol/THF (3:1, 4 mL). Then, di-tert-butyl dicarbonate (105 μL, 1 eq) and DMAP (26.4 mg, 0.3 eq) were added and stirred for 16 hours under a reflux condition. The reactant was concentrated, diluted with EtOAc, washed with saturated NaCl aqueous solution, dried over anhydrous MgSO₄, and filtered. The collected organic solvents were concentrated under reduced pressure and separated and purified using silica gel column chromatography to obtain the desired white solid, 3-(tert-butoxycarbonyl)benzoic acid (50 mg, 0.458 mmol, 49%).

¹H NMR (400 MHz, DMSO-d₆) δ 13.29 (s, 1H), 8.43 (t, J=1.8 Hz, 1H), 8.17 (dt, J=7.7, 1.6 Hz, 1H), 8.13 (dq, J=7.8, 1.6 Hz, 1H), 7.64 (t, J=7.7 Hz, 1H), 1.57 (s, 9H).

### Synthesis of tert-butyl 3-((2,6-dioxopiperidin-3-yl)carbamoyl)benzoate (step 2)

3-aminopiperidine-2,6-dione hydrochloride (40.7 mg, 0.247 mmol, 1.1 eq), 3-(tert-butoxycarbonyl)benzoic acid (50 mg, 1 eq), EDCI-HCl (43.1 mg, 1 eq), and HOBt (34.5 mg, 1 eq) were dissolved in DMF (1 mL). Then, DIPEA (98 μL, 2.5 eq) was added, and the mixture was stirred at room temperature for 16 hours. The reactant was concentrated, diluted with EtOAc, washed with saturated NaCl aqueous solution, dried over anhydrous MgSO₄, and filtered. The filtrate was concentrated under reduced pressure and then separated and purified using silica gel column chromatography to obtain the desired light blue solid, tert-butyl 3-((2,6-dioxopiperidin-3-yl)carbamoyl)benzoate (50 mg, 0.150 mmol, 66%).

¹H NMR (500 MHz, DMSO-d₆) δ 10.91 (s, 1H), 9.01 (d, J=8.4 Hz, 1H), 8.40 (s, 1H), 8.15-8.05 (m, 2H), 7.72-7.59 (m, 1H), 4.87-4.75 (m, 1H), 2.90-2.74 (m, 1H), 2.60-2.54 (m, 1H), 2.19-2.10 (m, 1H), 2.05-1.97 (m, 1H), 1.58 (s, 9H).

### Synthesis of 3-((2,6-dioxopiperidin-3-yl)carbamoyl)benzoic acid (step 3)

Tert-butyl 3-((2,6-dioxopiperidin-3-yl)carbamoyl)benzoate (50 mg, 0.150 mmol, 1 eq) was dissolved in 50% TFA/DCM (2 mL) and the mixture was stirred at room temperature for 1 hour. The reactant was concentrated under reduced pressure to obtain the desired solid, 3-((2,6-dioxopiperidin-3-yl)carbamoyl)benzoic acid (40 mg, 0.145 mmol, 96%).

¹H NMR (500 MHz, DMSO-d₆) δ 10.89 (s, 1H), 9.00 (d, J=8.4 Hz, 1H), 8.48 (t, J=1.8 Hz, 1H), 8.11 (dd, J=7.8, 1.7 Hz, 2H), 7.82-7.56 (m, 1H), 4.92-4.77 (m, 1H), 2.89-2.73 (m, 1H), 2.59-2.52 (m, 1H), 2.19-2.06 (m, 1H), 2.02-1.97 (m, 1H).

### Synthesis of 5-((2,6-dioxopiperidin-3-yl)carbamoyl)nicotinic acid

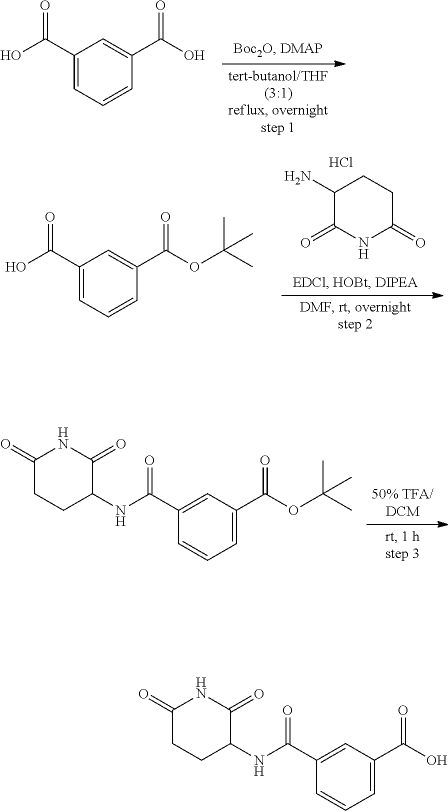 [SMILES: O=C(O)c1cccc(C(=O)O)c1 ; Cl ; CC(C)(C)OC(=O)c1cccc(C(=O)O)c1 ; NC1CCC(=O)NC1=O ; CC(C)(C)OC(=O)c1cccc(C(=O)NC2CCC(=O)NC2=O)c1 ; O=C1CCC(NC(=O)c2cccc(C(=O)O)c2)C(=O)N1]

### Synthesis of 5-(tert-butoxycarbonyl)nicotinic acid (step 1)

Pyridine-3,5-dicarboxylic acid (153 mg, 0.916 mmol, 2 eq) was dissolved in tert-butanol/THF (3:1, 4 mL). Then, di-tert-butyl dicarbonate (105 μL, 1 eq) and DMAP (26.4 mg, 0.3 eq) were added and stirred for 16 hours under a reflux condition. The reactant was concentrated, diluted with EtOAc, washed with saturated NaCl aqueous solution, dried over anhydrous MgSO₄, and filtered. The filtrate was separated and purified using column chromatography to obtain the desired white solid, 5-(tert-butoxycarbonyl)nicotinic acid (12 mg, 0.054 mmol, 11%).

¹H NMR (400 MHz, DMSO-d₆) δ 9.22 (dd, J=11.1, 2.1 Hz, 2H), 8.58 (t, J=2.1 Hz, 1H), 1.58 (s, 9H).

### Synthesis of tert-butyl 5-((2,6-dioxopiperidin-3-yl)carbamoyl)nicotinate (step 2)

3-aminopiperidine-2,6-dione hydrochloride (324 mg, 0.247 mmol, 1.1 eq), 5-(tert-butoxycarbonyl)nicotinic acid (400 mg, 1 eq), EDCI-HCl (344 mg, 1 eq), and HOBt (274 mg, 1 eq) were dissolved in DMF (7 mL). Then, DIPEA (780 μL, 2.5 eq) was added, and the mixture was stirred at room temperature for 16 hours. The reactant was concentrated, diluted with EtOAc, washed with saturated NaCl aqueous solution, dried over anhydrous MgSO₄, and filtered. The filtrate was concentrated under reduced pressure and then separated and purified using silica gel column chromatography to obtain the desired light blue solid, tert-butyl 5-((2,6-dioxopiperidin-3-yl)carbamoyl)nicotinate (350 mg, 1.050 mmol, 58%).

¹H NMR (500 MHz, DMSO-d₆) δ 10.94 (s, 1H), 9.25-9.21 (m, 2H), 9.18 (d, J=2.0 Hz, 1H), 8.65 (t, J=2.1 Hz, 1H), 4.90-4.79 (m, 1H), 2.89-2.79 (m, 1H), 2.61-2.55 (m, 1H), 2.19-2.10 (m, 1H), 2.07-1.98 (m, 1H), 1.60 (s, 9H).

### Synthesis of 5-((2,6-dioxopiperidin-3-yl)carbamoyl)nicotinic acid (step 3)

Tert-butyl 5-((2,6-dioxopiperidin-3-yl)carbamoyl)nicotinate (350 mg, 1.050 mmol, 1 eq) was dissolved in 50% TFA/DCM (8 mL), and the mixture was stirred at room temperature for 1 hour. The reactant was concentrated under reduced pressure to obtain the desired white solid, 5-((2,6-dioxopiperidin-3-yl)carbamoyl)nicotinic acid (290 mg, 1.046 mmol, 100%).

¹H NMR (500 MHz, DMSO-d₆) δ 10.93 (s, 1H), 9.30-9.12 (m, 3H), 8.73 (t, J=2.1 Hz, 1H), 4.92-4.80 (m, 1H), 2.86-2.77 (m, 1H), 2.59-2.53 (m, 1H), 2.17-2.08 (m, 1H), 2.06-1.96 (m, 1H).

### Synthesis of 2-(2,6-dioxopiperidin-3-yl)-5,6-difluoroisoindoline-1,3-dione

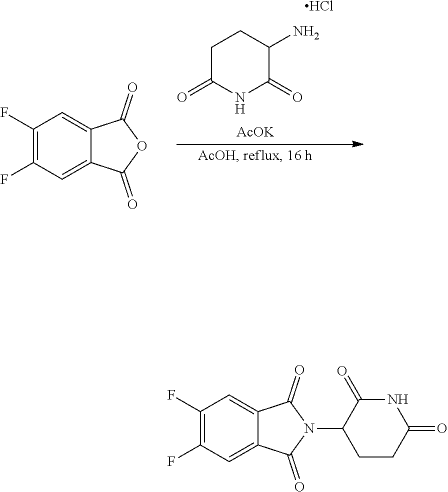 [SMILES: "AcOK" ; Cl ; O=C1OC(=O)c2cc(F)c(F)cc21 ; NC1CCC(=O)NC1=O ; O=C1CCC(N2C(=O)c3cc(F)c(F)cc3C2=O)C(=O)N1]

5,6-difluoroisobenzofuran-1,3-dione (10 g, 54.3 mmol) was added in acetic acid (70 mL). 3-aminopiperidine-2,6-dione hydrochloride (8.94 g, 54.3 mmol) and potassium acetate (16 g, 163 mmol) were added thereto at room temperature, and then reacted overnight under a reflux condition for 16 hours. The reaction solvent was concentrated, washed with distilled water and filtered to obtain the desired purple solid, 2-(2,6-dioxopiperidin-3-yl)-5,6-difluoroisoindoline-1,3-dione (12.5 g 42.5 mmol, 78%).

¹H NMR (400 MHz, DMSO-d₆) δ 11.16 (s, 1H), 8.16 (d, J=7.7 Hz, 1H), 8.14 (d, J=7.5 Hz, 1H), 5.17 (dd, J=12.9, 5.4 Hz, 1H), 2.98-2.80 (m, 1H), 2.68-2.56 (m, 1H), 2.48-2.42 (m, 1H), 2.12-2.00 (m, 1H).

### Synthesis of 2-(3-((1-(2,6-dioxopiperidin-3-yl)-2,5-dioxo-2,5-dihydro-1H-pyrrol-3-yl)amino)phenyl)acetic acid

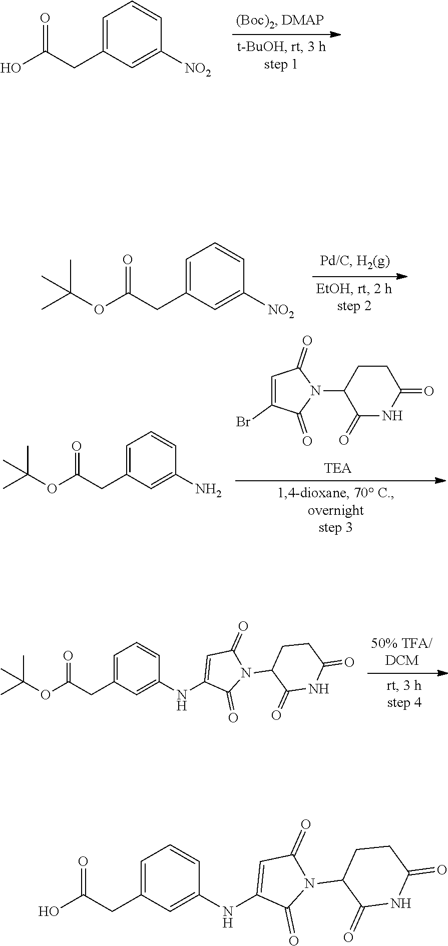 [SMILES: O=C(O)Cc1cccc([N+](=O)[O-])c1 ; CC(C)(C)OC(=O)Cc1cccc([N+](=O)[O-])c1 ; CC(C)(C)OC(=O)Cc1cccc(N)c1 ; O=C1CCC(N2C(=O)C=C(Br)C2=O)C(=O)N1 ; CC(C)(C)OC(=O)Cc1cccc(NC2=CC(=O)N(C3CCC(=O)NC3=O)C2=O)c1 ; O=C(O)Cc1cccc(NC2=CC(=O)N(C3CCC(=O)NC3=O)C2=O)c1]

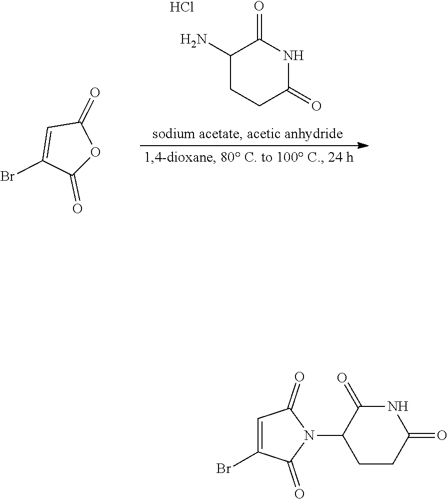 [SMILES: O=C1C=C(Br)C(=O)O1 ; "sodium acetate, acetic anhydride" ; Cl ; NC1CCC(=O)NC1=O ; O=C1CCC(N2C(=O)C=C(Br)C2=O)C(=O)N1]

### Synthesis of tert-butyl 2-(3-nitrophenyl)acetate (step 1)

2-(3-nitropheny)lacetic acid (10 g, 55.2 mmol, 1 eq), di-tert-butyl dicarbonate (24.1 g, 110 mmol, 2 eq), and DMAP (2.023 g, 16.56 mmol 0.3 eq) were added to t-BuOH (150 mL) and stirred at room temperature for 3 hours. After completion of the reaction, it is concentrated under reduced pressure. The collected organic solvents were concentrated and then purified by column chromatography to obtain a yellow oil, tert-butyl 2-(3-nitrophenyl)acetate (10.64 g, 44.8 mmol, 81%).

¹H NMR (300 MHz, CDCl₃) δ 8.16 (t, J=2.0 Hz, 1H), 8.13-8.11 (m, 1H), 7.64-7.61 (m, 1H), 7.50 (t, J=7.9 Hz, 1H), 3.66 (s, 2H), 1.46 (s, 9H).

### Synthesis of tert-butyl 2-(3-aminophenyl)acetate (step 2)

Tert-butyl 2-(3-nitrophenyl)acetate (10.36 g. 43.7 mmol, 1 eq) and 5% Pd/C (1.5 g, 14.10 mmol, 0.323 eq) were added in ethanol (70 mL), and stirred at room temperature for 2 hours using a Hydrogen Parr Shaker. After completion of the reaction, the metal catalyst was removed using a celite filter. The organic layers were collected and concentrated under reduced pressure to obtain tert-butyl 2-(3-aminophenyl)acetate (9 g, 43.4 mmol, 99%).

¹H NMR (300 MHz, CDCl₋₃) δ 7.09 (t, J=7.7 Hz, 1H), 6.70-6.63 (m, 1H), 6.61 (d, J=1.8 Hz, 1H), 6.57-6.56 (m, 1H), 3.64 (s, 2H), 3.43 (s, 2H), 1.44 (s, 9H).

### Synthesis of tert-butyl 2-(3-((1-(2,6-dioxopiperidin-3-yl)-2,5-dioxo-2,5-dihydro-1H-pyrrol-3-yl)amino)phenyl)acetate (step 3)

3-(3-bromo-2,5-dioxo-2,5-dihydro-1H-pyrrol-1-yl)piperidine-2,6-dione (5 g, 17.42 mmol, 1 eq), tert-butyl 2-(Add 3-aminophenyl)acetate (5.42 g, 26.1 mmol, 1.5 eq) and triethylamine hydrochloride (7.19 g, 52.3 mmol, 3 eq) were added to 1,4-dioxane (50 mL) and reacted at 70° C. for 12 hours. After completion of the reaction, the mixture was diluted with water and extracted with ethyl acetate. It was dried by adding anhydrous magnesium sulfate, and filtered. The organic layers were collected and concentrated under reduced pressure. The concentrate was purified by column chromatography to obtain tert-butyl 2-(3-((1-(2,6-dioxopiperidin-3-yl)-2,5-dioxo-2,5-dihydro-1H using column chromatography-pyrrol-3-yl)amino)phenyl)acetate (3 g 7.26 mmol, 41%).

¹H NMR (300 MHz, CDCl₃) δ 8.10 (s, 1H), 7.38 (d, J=7.9 Hz, 1H), 7.34 (d, J=2.8 Hz, 1H), 7.13 (d, J=2.1 Hz, 1H), 7.09 (d, J=7.9 Hz, 2H), 5.62 (s, 1H), 4.85 (m, 1H), 2.95-2.83 (m, 1H), 2.82-2.69 (m, 2H), 2.18-2.11 (m, 1H), 1.46 (s, 9H).

### Synthesis of 2-(3-((1-(2,6-dioxopiperidin-3-yl)-2,5-dioxo-2,5-dihydro-1H-pyrrol-3-yl)amino)phenyl)acetic acid (step 4)

Tert-butyl 2-(3-((1-(2,6-dioxopiperidin-3-yl)-2,5-dioxo-2,5-dihydro-1H-pyrrol-3 yl)amino)phenyl)acetate (50 mg, 0.121 mmol) was added in 1 ml of 50% TFA/DCM and stirred for 3 hours. After completion of the reaction, it is concentrated under reduced pressure. After washing with ether and drying, the target compound 2-(3-((1-(2,6-dioxopiperidin-3-yl)-2,5-dioxo-2,5-dihydro-1H-pyrrol-3-yl)amino)phenyl)acetic acid (39 mg, 0.108 mmol, 90%) was obtained.

¹H NMR (300 MHz, DMSO-d₆) δ 11.07 (s, 1H), 9.83 (s, 1H), 7.39-7.27 (m, 3H), 7.12 (d, J=14.3 Hz, 1H), 7.03-7.00 (m, 1H), 5.76 (s, 1H), 5.00-4.93 (m, 1H), 3.61 (d, J=3.3 Hz, 2H), 2.92-2.82 (m, 1H), 2.60-2.54 (m, 2H), 2.01 (s, 1H).

### Synthesis of 3-(3-bromo-2,5-dioxo-2,5-dihydro-1H-pyrrol-1-yl)piperidine-2,6-dione

3-aminopiperidine-2,6-dione hydrochloride (5 g, 30.4 mmol, 1.0 eq) and 3-bromofuran-2,5-dione (3.10 mL, 33.4 mmol, 1.1 eq) were added in 1,4-dioxane (50 mL). After stirring for 1 hour, sodium acetate (2.74 g, 33.4 mmol, 1.1 eq) was added. After stirring for 5 hours, acetic anhydride (2.87 mL, 30.4 mmol, 1 eq) was added and stirred for 15 hours. The reaction solvent was diluted with DCM, washed with water, dried over anhydrous MgSO₄, filtered and concentrated. The concentrate was separated and purified by silica gel column chromatography to obtain 3-(3-bromo-2,5-dioxo-2,5-dihydro-1H-pyrrol-1-yl)piperidine-2,6-dione (2.53 g, 8.81 mmol, 29%) as a yellow solid.

¹H NMR (300 MHz, CDCl₃) δ 8.89 (s, 1H), 6.99 (s, 1H), 4.92-4.82 (m, 1H), 2.93-2.84 (m, 1H), 2.82-2.66 (m, 2H), 2.15-2.06 (m, 1H).

### Synthesis of 3-(2,4-dioxotetrahydropyrimidin-1(2H)-yl)benzofuran-6-carboxylic acid

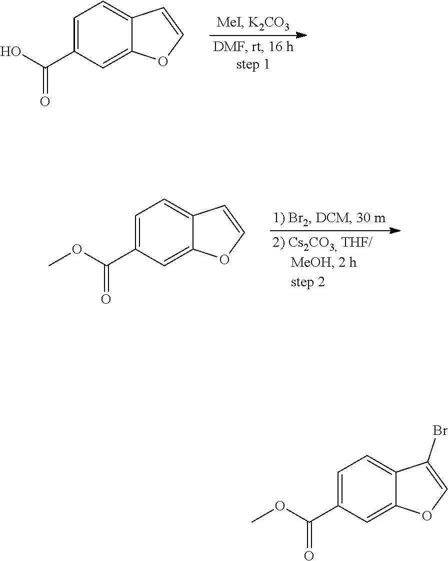

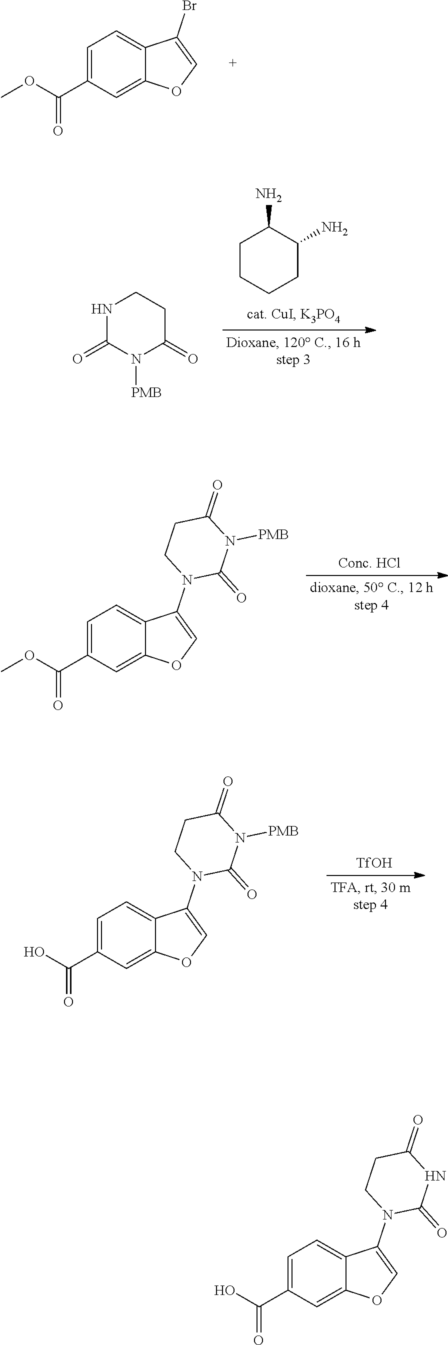

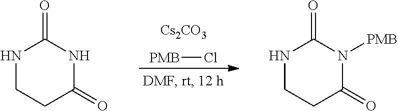

### Synthesis of Ethyl benzofuran-6-carboxylate (step 1)

Benzofuran-6-carboxylic acid (3.91 g, 24.11 mmol, 1 eq), methyl iodide (2.252 mL, 36.2 mmol, 1.5 eq), and K₂CO₃ (6.67 g, 48.2 mmol, 2 eq) were added to DMF (30 mL) and stirred at room temperature for 16 hours. After completion of the reaction, the reactant was diluted with water and then extracted with ethyl acetate. The organic layers were collected, dried with anhydrous magnesium sulfate and filtered. The collected organic layer was concentrated under reduced pressure, and used in the next step without further purification. Methyl benzofuran-6-carboxylate (3.73 g, 21.17 mmol, 88%) was obtained.

¹H NMR (300 MHz, CDCl₃) δ 8.21 (dd, J=1.6, 0.8 Hz, 1H), 7.96 (dd, J=8.2, 1.4 Hz, 1H), 7.76 (d, J=2.0 Hz, 1H), 7.63 (dd, J=8.2, 0.7 Hz, 1H), 6.82 (dd, J=2.2, 1.0 Hz, 1H), 3.95 (s, 3H).

### Synthesis of methyl 3-bromobenzofuran-6-carboxylate (step 2)

Ethyl benzofuran-6-carboxylate (3.73 g, 21.17 mmol, 1 eq) and bromine (2.17 mL, 42.3 mmol, 2 eq) were added to DCM (30 mL) and stirred for 15 minutes. After completion of the reaction, it was diluted with Na₂S₂O₃ and extracted using DCM. After the organic layers were collected, it was dried by adding anhydrous magnesium sulfate. The collected organic layer was concentrated under reduced pressure. After adding Cs₂CO₃ (13.8 g, 42.3 mmol, 2 eq), it was dissolved in THF (30 mL) and MeOH (6 mL). The reactant was stirred at room temperature for 2 hours. After completion of the reaction, the reactant was diluted with water and then extracted using EA. The organic layer was collected and dried using anhydrous magnesium sulfate. After filtering, the organic layer was collected and concentrated under reduced pressure. The concentrate is separated using column chromatography to obtain the target compound, methyl 3-bromobenzofuran-6-carboxylate (3.14 g, 12.31 mmol, 58%).

¹H NMR (300 MHz, CDCl₃) δ 8.18 (d, J=1.3 Hz, 1H), 8.02 (dd, J=8.2, 1.3 Hz, 1H), 7.77 (s, 1H), 7.57 (d, J=8.2 Hz, 1H), 3.95 (s, 3H).

### Synthesis of methyl 3-(3-(4-methoxybenzyl)-2,4-dioxotetrahydropyrimidin-1(2H)-yl) benzofuran-6-carboxylate (step 3)

Methyl 3-bromobenzofuran-6-carboxylate (1.00 g, 3.92 mmol, 1 eq), 3-(4-methoxybenzyl)dihydropyrimidine-2,4(1H,3H)-dione (1.19 g, 5.10 mmol, 1.3 eq), potassium phosphate (1.66 g, 7.84 mmol, 2 eq) and CuI (373 mg, 1.96 mmol, 0.5 eq) were added to 1,4-dioxane (20 mL). (1R,2R)-cyclohexane-1,2-diamine (235 uL, 1.96 mmol, 0.5 eq) was added, and nitrogen purging was performed for 5 minutes. The reactant was stirred at 120° C. for 12 hours. After completion of the reaction, the reactant was filtered with celite and washed with MeOH. The organic layer was collected, concentrated under reduced pressure, and separated using column chromatography to obtain methyl 3-(3-(4-methoxybenzyl)-2,4-dioxotetrahydropyrimidin-1(2H)-yl) benzofuran-6-carboxylate (810 mg, 1.98 mmol, 51%).

¹H NMR (500 MHz, CDCl₃) δ 8.19 (s, 1H), 7.98 (dd, J=8.2, 1.4 Hz, 1H), 7.87 (s, 1H), 7.49 (d, J=8.3 Hz, 1H), 7.43 (d, J=2.0 Hz, 1H), 7.42 (d, J=2.0 Hz, 1H), 6.85 (d, J=2.0 Hz, 1H), 6.83 (d, J=2.2 Hz, 1H), 4.99 (s, 2H), 3.95 (s, 3H), 3.86 (t, J=6.7 Hz, 2H), 3.79 (s, 3H), 2.96 (t, J=6.7 Hz, 2H).

### Synthesis of 3-(3-(4-methoxybenzyl)-2,4-dioxotetrahydropyrimidin-1(2H)-yl)benzofuran-6-carboxylic acid (step 4)

Methyl 3-(3-(4-methoxybenzyl)-2,4-dioxotetrahydropyrimidin-1(2H)-yl)benzofuran-6-carboxylate (500 mg, 1.224 mmol, 1 eq) was added in 1,4 dioxane (5 mL). Then, concentrated HCl (5 mL) was added dropwise at 0° C. After raising the temperature from 0° C. to 50° C., the mixture was stirred for 12 hours. After completion of the reaction, the reactant was concentrated and separated by column chromatography to obtain 3-(3-(4-methoxybenzyl)-2,4-dioxotetrahydropyrimidin-1(2H)-yl)benzofuran-6-carboxylic acid (182 mg, 0.461 mmol, 38%).

1H NMR (500 MHz, MeOD) δ 8.16 (d, J=1.3 Hz, 1H), 8.11 (s, 1H), 7.95 (dd, J=8.3, 1.4 Hz, 1H), 7.58 (d, J=8.2 Hz, 1H), 7.33 (d, J=2.1 Hz, 1H), 7.32 (d, J=2.2 Hz, 1H), 6.86 (d, J=2.1 Hz, 1H), 6.85 (d, J=2.2 Hz, 1H), 4.95 (s, 2H), 3.90 (t, J=6.7 Hz, 2H), 3.77 (s, 3H), 2.99 (t, J=6.7 Hz, 2H).

### Synthesis of 3-(2,4-dioxotetrahydropyrimidin-1(2H)-yl)benzofuran-6-carboxylic acid (step 5)

3-(3-(4-methoxybenzyl)-2,4-dioxotetrahydropyrimidin-1(2H)-yl)-2,3-dihydrobenzofuran-6-carboxylic acid (182 mg, 0.461 mmol, 1 eq) was added to TFA (1 mL), and TfOH (0.5 mL) was added. The mixture was reacted for 1 hour at room temperature. After completion of the reaction, the mixture was diluted with an aqueous NaHCO₃ solution and extracted using EA. After the organic layer was collected, its water was dried by adding anhydrous magnesium sulfate, and filtered. After concentrating the organic layer under reduced pressure, the concentrate was separated by column chromatography to obtain 3-(2,4-dioxotetrahydropyrimidin-1(2H)-yl)benzofuran-6-carboxylic acid (81 mg, 0.295 mmol, 64%).

¹H NMR (300 MHz, DMSO-d₆) δ 10.60 (s, 1H), 8.34 (s, 1H), 8.12 (d, J=1.3 Hz, 1H), 7.88 (dd, J=8.2, 1.4 Hz, 1H), 7.72 (d, J=8.3 Hz, 1H), 3.87 (t, J=6.7 Hz, 2H), 2.80 (t, J=6.7 Hz, 2H).

### Synthesis of 3-(4-methoxybenzyl)dihydropyrimidine-2,4(1H,3H)-dione

Dihydropyrimidine-2,4(1H,3H)-dione (5 g, 43.8 mmol, 1 eq), 4-methoxybenzyl chloride (7.72 mL, 57 mmol., 1.3 eq), and cesium carbonate (17.13 g, 52.6 mmol, 1.2 eq) were added in DMF (20 mL) and the mixture was stirred at room temperature for 12 hours. After completion of the reaction, the reactant was filtered and washed with DMF. After concentrating the reactant under reduced pressure, water was added. After filtering the solidified compound, the compound was washed with EA/Hexane (1:1) and DCM and dried to obtain 3-(4-methoxybenzyl)dihydropyrimidine-2,4(1H,3H)-dione (6.2 g, 26.5 mmol, 60%).

¹H NMR (500 MHz, DMSO-d₆) δ 7.82 (s, 1H), 7.18 (d, J=2.0 Hz, 1H), 7.16 (d, J=2.1 Hz, 1H), 6.85 (d, J=2.1 Hz, 1H), 6.83 (d, J=2.1 Hz, 1H), 4.71 (s, 2H), 3.71 (s, 3H), 3.21 (td, J=6.8, 2.7 Hz, 2H), 2.62 (t, J=6.8 Hz, 2H).

### Synthesis of 3-(2,4-dioxotetrahydropyrimidin-1 (2H)-yl)-4-methoxybenzoic acid

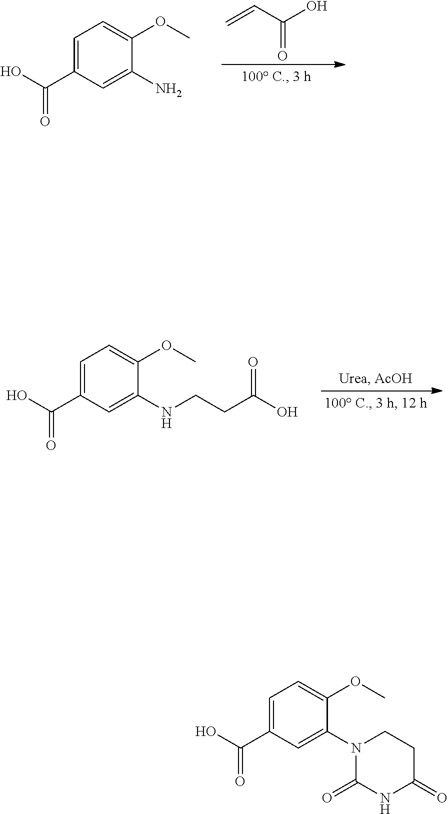

### 3-((2-carboxyethyl)amino)-4-methoxybenzoic acid (step 1)

Acrylic acid (8.05 mL, 117 mmol) was added to 3-amino-4-methoxybenzoic acid (5.0 g, 29 mmol), and the mixture was stirred at 100° C. for 3 hours. After completion of the reaction, the reactant was cooled to room temperature, and a gray suspension was obtained and used for the next reaction without purification.

### 3-(2,4-dioxotetrahydropyrimidin-1(2H)-yl)-4-methoxybenzoic acid (step 2)

Acetic acid (35 ml) was added to 3-((2-carboxyethyl)amino)-4-methoxybenzoic acid, and the mixture was stirred at 100° C. for 10 minutes. Urea (11.3 g, 188 mmol, 6.5 eq) was added and stirred at 120° C. for 12 hours. After completion of the reaction, the reactant was cooled to room temperature, and then 1 N HCl (150 mL) and H₂O (50 mL) were added. The reaction was held at 5° C. for 12 hours. After filtering the generated solid, it was washed with 0.05 N HCl and water. The resulting solid was dried to obtain a pale yellow 3-(2,4-dioxotetrahydropyrimidin-1(2H)-yl)-4-methoxybenzoic acid (6.64 g, 25.1 mmol, 84%).

¹H NMR (300 MHz, DMSO-d₆) δ 12.75 (s, 1H), 10.37 (s, 1H), 7.92 (dd, J=8.6, 2.2 Hz, 1H), 7.83 (d, J=2.2 Hz, 1H), 7.21 (d, J=8.7 Hz, 1H), 3.88 (s, 3H), 3.60 (t, J=6.7 Hz, 2H), 2.69 (t, J=6.8 Hz, 2H).

The compounds of Tables 1 and 2 were prepared in the same or similar manner to the previous preparation methods.

The NMR measurement results of the compounds of Examples or Comparative Examples prepared according to the present invention are summarized in Table 3 below.

TABLE 3

| Compound | NMR and MS Data |
|---|---|
| TDH-066 | ¹H NMR (400 MHz, MeOD) δ 8.39-8.35 (m, 1H), 8.34-8.30 (m, 1H), 7.72-7.65 (m, 2H), |
| | 7.61-7.53 (m, 2H), 7.53-7.42 (m, 3H), 7.11-7.01 (m, 2H), 6.27 (s, 1H), 6.05 (s, 1H), |
| | 4.09-4.04 (m, 2H), 3.70-3.62 (m, 2H), 2.55-2.45 (m, 2H), 1.51 (s, 9H). |
| TDH-067 | ¹H NMR (300 MHz, Chloroform-d) δ 8.73 (d, J = 6.9 Hz, 1H), 8.23 (s, 1H), 7.53 (d, J = 7.0 |
| | Hz, 2H), 7.50-7.36 (m, 7H), 7.00 (t, J = 8.6 Hz, 2H), 5.63 (d, J = 6.9 Hz, 2H). |
| TDH-068 | ¹H NMR (300 MHz, DMSO-d₆) δ 10.76 (s, 1H), 8.72 (s, 1H), 8.25 (s, 1H), 7.99 (s, 1H), 7.77- |
| | 7.72 (m, 2H), 7.66-7.60 (m, 2H), 7.47 (dt, J = 13.8, 7.0 Hz, 4H), 7.18-7.12 (m, 2H), 6.27 (s, |
| | 1H), 4.60-4.51 (m, 1H), 3.45-3.35 (m, 1H), 2.24-2.17 (m, 4H). |
| TDH-069 | 1H NMR (400 MHz, MeOD) δ 8.60 (s, 1H), 8.28 (s, 1H), 8.08 (s, 1H), 7.77-7.71 (m, 2H), |
| | 7.63-7.55 (m, 2H), 7.50-7.45 (m, 3H), 7.07 (t, J = 8.8 Hz, 2H), 6.36 (s, 1H), 4.67-4.60 (m, |
| | 1H), 3.61-3.57 (m, 2H), 3.29-3.23 (m, 2H), 2.38-2.32 (m, 4H); LC/MS (ESI) m/z 516 [M + H]+ |
| TDH-070 | LC/MS (ESI) m/z 447 [M + H]+ |
| TDH-071 | 1H NMR (300 MHz, DMSO-d6) δ 10.58 (s, 1H), 8.99-8.92 (m, 1H), 8.29-8.26 (m, 1H), |
| | 7.91-7.88 (m, 1H), 7.64-7.57 (m, 2H), 7.55-7.51 (m, 3H), 7.44-7.32 (m, 4H), 7.20-7.12 |
| | (m, 2H), 5.77-5.74 (m, 1H). |
| TDH-072 | ¹H NMR (300 MHz, MeOD) δ 8.40-8.35 (m, 1H), 8.30-8.25 (m, 1H), 7.70-7.64 (m, 2H), |
| | 7.64-7.59 (m, 1H), 7.59-7.51 (m, 2H), 7.51-7.39 (m, 3H), 7.06-6.96 (m, 3H), 6.89-6.82 |
| | (m, 1H), 6.29-6.25 (m, 1H), 6.07-6.00 (m, 1H), 5.05-4.94 (m, 1H), 4.23-4.08 (m, 2H), |
| | 3.88 (s, 2H), 3.83-3.75 (m, 1H), 3.74-3.65 (m, 2H), 3.30-3.23 (m, 2H), 2.80-2.69 (m, |
| | 3H), 2.59-2.53 (m, 1H), 2.53-2.46 (m, 1H), 2.43-2.30 (m, 2H), 2.17-2.02 (m, 2H), 1.67- |
| | 1.48 (m, 5H). |
| TDH-073 | ¹H NMR (300 MHz, MeOD) δ 8.37-8.33 (m, 1H), 8.27-8.24 (m, 1H), 7.70-7.64 (m, 2H), |
| | 7.60-7.51 (m, 3H), 7.49-7.42 (m, 3H), 7.09-6.94 (m, 4H), 6.32-6.24 (m, 1H), 6.09-5.98 |
| | (m, 1H), 5.03-4.93 (m, 1H), 4.20 (s, 2H), 3.86-3.79 (m, 1H), 3.79-3.67 (m, 2H), 2.84- |
| | 2.69 (m, 3H), 2.63-2.41 (m, 5H), 2.17-2.08 (m, 1H), 1.78-1.63 (m, 4H), 1.57-1.45 (m, 2H). |
| TDH-074 | ¹H NMR (300 MHz, MeOD) δ 8.44-8.33 (m, 1H), 8.33-8.23 (m, 1H), 7.70-7.61 (m, 3H), |
| | 7.61-7.52 (m, 2H), 7.51-7.39 (m, 3H), 7.32-7.24 (m, 1H), 7.22-7.07 (m, 1H), 7.07-6.96 |
| | (m, 2H), 6.30-6.21 (m, 1H), 6.16-5.99 (m, 1H), 5.09-4.97 (m, 1H), 4.24-4.15 (m, 2H), |
| | 4.07-3.97 (m, 2H), 3.84-3.68 (m, 2H), 3.27-3.11 (m, 2H), 3.06-2.94 (m, 2H), 2.89-2.67 |
| | (m, 3H), 2.62-2.55 (m, 1H), 2.52-2.41 (m, 3H), 2.18-2.08 (m, 1H), 1.89-1.75 (m, 3H), |
| | 1.72-1.63 (m, 2H), 1.61-1.47 (m, 3H), 1.45-1.33 (m, 3H). |
| TDH-075 | ¹H NMR (300 MHz, MeOD) 0 8.41-8.27 (m, 2H), 7.80-7.64 (m, 3H), 7.64-7.44 (m, 6H), |
| | 7.17-7.04 (m, 2H), 7.04-6.91 (m, 1H), 6.89-6.74 (m, 1H), 6.27 (s, 1H), 6.16-6.01 (m, |
| | 1H), 5.11-4.98 (m, 1H), 4.30-4.14 (m, 2H), 3.87-3.69 (m, 2H), 3.27-3.14 (m, 2H), 2.90- |
| | 2.63 (m, 2H), 2.63-2.41 (m, 3H), 2.16-1.99 (m, 1H), 1.84-1.58 (m, 4H), 1.58-1.41 (m, |
| | 2H), 1.41-1.20 (m, 7H). |
| TDH-078 | ¹H NMR (300 MHz, DMSO) δ 11.08 (s, 1H), 10.58 (s, 1H), 8.47-8.38 (m, 1H), 8.19-8.11 |
| | (m, 1H), 8.08-7.99 (m, 1H), 7.72-7.64 (m, 2H), 7.64-7.53 (m, 2H), 7.53-7.39 (m, 3H), |
| | 7.25 (s, 2H), 7.21-7.10 (m, 2H), 6.84-6.76 (m, 1H), 6.22 (s, 1H), 6.07 (s, 1H), 5.12-5.00 |
| | (m, 1H), 4.39-4.27 (m, 2H), 4.20-4.02 (m, 3H), 3.71-3.57 (m, 2H), 3.48-3.38 (m, 2H), |
| | 3.16-3.02 (m, 2H), 2.96-2.73 (m, 2H), 2.42-2.27 (m, 3H), 2.06-1.94 (m, 1H), 1.60-1.37 |
| | (m, 4H), 1.37-1.22 (m, 2H), 1.13-1.02 (m, 2H). |
| TDH-079 | ¹H NMR (300 MHz, DMSO) δ 11.12 (s, 1H), 10.60 (s, 1H), 8.46-8.36 (m, 1H), 8.22-8.04 |
| | (m, 2H), 7.74-7.64 (m, 2H), 7.64-7.54 (m, 3H), 7.54-7.38 (m, 3H), 7.35-7.22 (m, 2H), |
| | 7.22-7.12 (m, 2H), 7.12-7.02 (m, 1H), 7.01-6.92 (m, 1H), 6.86 (d, J = 8.6 Hz, 1H), 6.23 |
| | (s, 1H), 6.12-6.03 (m, 1H), 5.15-5.04 (m, 1H), 4.17-4.05 (m, 2H), 3.92 (d, J = 5.6 Hz, |
| | 2H), 3.71-3.58 (m, 2H), 3.22-3.15 (m, 1H), 3.15-3.04 (m, 2H), 2.98-2.82 (m, 1H), 2.67- |
| | 2.54 (m, 2H), 2.44-2.24 (m, 3H), 2.11-1.96 (m, 1H), 1.59-1.35 (m, 4H), 1.35-1.20 (m, 2H). |
| TDH-080 | ¹H NMR (300 MHz, DMSO) δ 11.10 (s, 1H), 10.59 (s, 1H), 8.46-8.37 (m, 1H), 8.20-8.11 |
| | (m, 1H), 7.87-7.76 (m, 1H), 7.75-7.64 (m, 3H), 7.64-7.53 (m, 2H), 7.53-7.37 (m, 3H), |
| | 7.37-7.31 (m, 2H), 7.30-7.21 (m, 2H), 7.21-7.09 (m, 2H), 6.23 (s, 1H), 6.13-6.04 (m, |
| | 1H), 5.16-5.04 (m, 1H), 4.19-4.05 (m, 2H), 3.79-3.59 (m, 4H), 3.22-3.14 (m, 1H), 3.12- |
| | 2.99 (m, 2H), 2.99-2.80 (m, 3H), 2.65-2.54 (m, 2H), 2.44-2.24 (m, 4H), 2.12-2.01 (m, |
| | 1H), 1.86-1.71 (m, 4H), 1.59-1.35 (m, 4H), 1.35-1.23 (m, 2H). |
| TDH-081 | ¹H NMR (300 MHz, DMSO) δ 11.13 (s, 1H), 10.58 (s, 1H), 8.46-8.36 (m, 1H), 8.24-8.11 |
| | (m, 1H), 8.04-7.92 (m, 1H), 7.92-7.76 (m, 1H), 7.76-7.65 (m, 2H), 7.65-7.55 (m, 2H), |
| | 7.55-7.34 (m, 5H), 7.34-7.23 (m, 2H), 7.23-7.09 (m, 2H), 6.22 (s, 1H), 6.17-6.02 (m, |
| | 1H), 5.22-5.06 (m, 1H), 4.77 (s, 2H), 4.19-3.98 (m, 2H), 3.72-3.53 (m, 2H), 3.21-3.08 |
| | (m, 2H), 3.01-2.80 (m, 1H), 2.72-2.54 (m, 2H), 2.47-2.25 (m, 4H), 2.11-1.99 (m, 1H), |
| | 1.60-1.37 (m, 4H), 1.37-1.20 (m, 2H). |
| TDH-082 | ¹H NMR (300 MHz, DMSO) δ 11.10 (s, 1H), 10.57 (s, 1H), 8.47-8.36 (m, 1H), 8.18-8.07 |
| | (m, 1H), 7.75-7.63 (m, 2H), 7.64-7.52 (m, 3H), 7.52-7.36 (m, 3H), 7.32-7.22 (m, 2H), |
| | 7.22-7.06 (m, 3H), 7.04-6.95 (m, 1H), 6.67-6.51 (m, 1H), 6.22 (s, 1H), 6.14-5.97 (m, |
| | 1H), 5.13-4.98 (m, 1H), 4.33-4.15 (m, 2H), 4.07 (s, 2H), 3.69-3.53 (m, 7H), 3.51-3.41 |
| | (m, 2H), 2.94-2.77 (m, 1H), 2.63-2.57 (m, 1H), 2.56-2.52 (m, 4H), 2.07-1.94 (m, 1H). |
| TDH-083 | ¹H NMR (300 MHz, DMSO) δ 11.07 (s, 1H), 10.57 (s, 1H), 8.40 (s, 1H), 8.20-8.08 (m, |
| | 1H), 7.73-7.66 (m, 2H), 7.63-7.42 (m, 6H), 7.31-7.22 (m, 2H), 7.21-7.12 (m, 3H), 7.07- |
| | 6.97 (m, 1H), 6.58-6.49 (m, 1H), 6.22 (s, 1H), 6.13-6.01 (m, 1H), 5.10-4.99 (m, 1H), |
| | 4.27-4.14 (m, 2H), 4.08 (s, 2H), 3.64-3.58 (m, 7H), 3.44-3.37 (m, 2H), 2.96-2.86 (m, |
| | 1H), 2.77-2.69 (m, 2H), 2.32-2.23 (m, 2H), 2.05-1.90 (m, 1H). |
| TDH-084 | ¹H NMR (300 MHz, DMSO) δ 11.11 (s, 1H), 10.59 (s, 1H), 8.44-8.37 (m, 1H), 8.26-8.09 |
| | (m, 2H), 7.73-7.64 (m, 2H), 7.64-7.53 (m, 3H), 7.53-7.37 (m, 3H), 7.34-7.23 (m, 2H), |
| | 7.23-7.10 (m, 2H), 7.10-7.02 (m, 1H), 6.99-6.92 (m, 1H), 6.90-6.81 (m, 1H), 6.22 (s, |
| | 1H), 6.14-6.00 (m, 1H), 5.13-5.00 (m, 1H), 4.28-4.15 (m, 2H), 4.15-4.05 (m, 2H), 3.99- |
| | 3.87 (m, 2H), 3.74-3.62 (m, 1H), 3.62-3.49 (m, 5H), 3.49-3.40 (m, 2H), 3.30-3.21 (m, |
| | 2H), 2.97-2.82 (m, 1H), 2.66-2.54 (m, 2H), 2.45-2.34 (m, 1H), 2.11-1.97 (m, 1H). |
| TDH-085 | ¹H NMR (300 MHz, DMSO) δ 11.07 (s, 1H), 10.56 (s, 1H), 8.52-8.36 (m, 1H), 8.19-8.08 |
| | (m, 2H), 7.73-7.65 (m, 2H), 7.64-7.55 (m, 3H), 7.54-7.41 (m, 3H), 7.41-7.30 (m, 1H), |
| | 7.30-7.22 (m, 2H), 7.22-7.07 (m, 2H), 6.96-6.91 (m, 1H), 6.91-6.75 (m, 1H), 6.22 (s, |
| | 1H), 6.13-5.99 (m, 1H), 5.12-4.98 (m, 1H), 4.26-4.15 (m, 2H), 4.15-4.06 (m, 2H), 3.92- |
| | 3.81 (m, 2H), 3.71-3.63 (m, 1H), 3.63-3.49 (m, 5H), 3.49-3.40 (m, 2H), 3.32-3.21 (m, |
| | 2H), 2.95-2.79 (m, 1H), 2.64-2.53 (m, 2H), 2.46-2.33 (m, 1H), 2.07-1.94 (m, 1H). |
| TDH-086 | ¹H NMR (300 MHz, DMSO) δ 11.08 (s, 1H), 10.55 (s, 1H), 8.41 (s, 1H), 8.26-8.11 (m, |
| | 2H), 7.73-7.53 (m, 4H), 7.53-7.38 (m, 2H), 7.30-7.22 (m, 1H), 7.22-7.09 (m, 2H), 6.88- |
| | 6.75 (m, 1H), 6.67-6.58 (m, 1H), 6.58-6.50 (m, 1H), 6.22 (s, 1H), 6.13-6.01 (m, 1H), |
| | 5.12-5.00 (m, 1H), 4.28-3.95 (m, 5H), 3.72-3.51 (m, 5H), 3.51-3.39 (m, 3H), 3.29-3.20 |
| | (m, 1H), 2.88-2.80 (m, 1H), 2.07-1.91 (m, 2H), 1.37-1.19 (m, 3H). |
| TDH-087 | ¹H NMR (300 MHz, DMSO) δ 11.08 (s, 1H), 10.56 (s, 1H), 8.48-8.36 (m, 1H), 8.22-8.08 |
| | (m, 2H), 7.73-7.64 (m, 2H), 7.63-7.52 (m, 2H), 7.52-7.38 (m, 2H), 7.30-7.22 (m, 1H), |
| | 7.20-7.09 (m, 2H), 6.86-6.75 (m, 1H), 6.21 (s, 1H), 6.13-6.01 (m, 1H), 5.11-5.00 (m, |
| | 1H), 4.34-4.06 (m, 5H), 3.73-3.51 (m, 5H), 3.53-3.38 (m, 3H), 3.30-3.20 (m, 2H), 2.96- |
| | 2.84 (m, 1H), 2.66-2.54 (m, 2H), 2.46-2.36 (m, 1H), 2.08-1.89 (m, 2H), 1.33-1.20 (m, 3H). |
| TDH-088 | ¹H NMR (300 MHz, DMSO) δ 11.09 (s, 1H), 10.55 (s, 1H), 8.47-8.38 (m, 1H), 8.21-8.07 |
| | (m, 1H), 8.00-7.85 (m, 1H), 7.76-7.54 (m, 4H), 7.55-7.41 (m, 3H), 7.41-7.23 (m, 4H), |
| | 7.23-7.07 (m, 2H), 6.21 (s, 1H), 6.15-6.02 (m, 1H), 5.17-5.04 (m, 1H), 4.30-4.16 (m, |
| | 2H), 4.16-4.01 (m, 2H), 3.79-3.52 (m, 7H), 3.52-3.39 (m, 3H), 3.27-3.12 (m, 2H), 2.95- |
| | 2.82 (m, 2H), 2.68-2.55 (m, 2H), 2.44-2.26 (m, 2H), 2.12-1.95 (m, 1H), 1.88-1.67 (m, 4H). |
| TDH-089 | ¹H NMR (300 MHz, DMSO) δ 11.09 (s, 1H), 10.56 (s, 1H), 8.46-8.34 (m, 1H), 8.19-8.10 |
| | (m, 1H), 7.97-7.84 (m, 1H), 7.69-7.56 (m, 4H), 7.52-7.41 (m, 3H), 7.35-7.10 (m, 6H), |
| | 6.22 (s, 1H), 6.15-6.02 (m, 1H), 5.13-5.01 (m, 1H), 4.28-4.15 (m, 2H), 4.08-4.02 (m, |
| | 2H), 3.75-3.50 (m, 7H), 3.47-3.37 (m, 3H), 3.23-3.16 (m, 2H), 2.98-2.88 (m, 2H), 2.70- |
| | 2.56 (m, 2H), 2.46-2.35 (m, 2H), 2.10-1.97 (m, 1H), 1.83-1.69 (m, 2H), 1.69-1.49 (m, |
| | 2H), 1.29-1.22 (m, 3H). |
| TDH-092 | ¹H NMR (300 MHz, DMSO) δ 11.07 (s, 1H), 10.57 (s, 1H), 8.43-8.37 (m, 1H), 8.21-8.14 |
| | (m, 1H), 8.10-8.03 (m, 1H), 7.72-7.56 (m, 3H), 7.52-7.40 (m, 2H), 7.40-7.23 (m, 2H), |
| | 7.23-7.09 (m, 2H), 6.84-6.79 (m, 1H), 6.72-6.60 (m, 1H), 6.23 (s, 1H), 6.09 (s, 1H), |
| | 5.13-4.96 (m, 1H), 4.20-3.98 (m, 3H), 3.69-3.59 (m, 3H), 3.19-3.08 (m, 3H), 2.95-2.81 |
| | (m, 2H), 2.42-2.27 (m, 3H), 2.10-1.95 (m, 2H), 1.63-1.39 (m, 3H), 1.28-1.26 (m, 6H). |
| TDH-095 | ¹H NMR (400 MHz, CDCl₃) δ 8.54 (s, 1H), 8.03-7.91 (m, 2H), 7.74-7.63 (m, 2H), 7.61- |
| | 7.49 (m, 3H), 7.49-7.36 (m, 4H), 7.14-7.08 (m, 1H), 7.03 (t, J = 8.3 Hz, 2H), 6.90 (d, J = |
| | 8.6 Hz, 1H), 6.46 (s, 2H), 6.25 (t, J = 5.5 Hz, 1H), 4.97-4.88 (m, 1H), 4.35 (s, 2H), 3.33- |
| | 3.23 (m, 3H), 3.01-2.70 (m, 6H), 2.48-2.19 (m, 5H), 2.19-2.07 (m, 2H), 1.76-1.65 (m, |
| | 4H), 1.50-1.31 (m, 14H). |
| TDH-096 | ¹H NMR (300 MHz, CDCl₃) δ 8.47 (s, 1H), 8.03 (d, J = 3.6 Hz, 1H), 7.91 (s, 1H), 7.63- |
| | 7.56 (m, 2H), 7.55-7.42 (m, 3H), 7.40-7.31 (m, 3H), 7.13-7.04 (m, 1H), 7.00-6.89 (m, |
| | 2H), 6.84-6.72 (m, 1H), 6.38 (s, 1H), 4.86-4.74 (m, 1H), 4.37-4.13 (m, 1H), 3.65-3.51 |
| | (m, 4H), 3.51-3.40 (m, 5H), 2.83-2.53 (m, 6H), 2.34-2.06 (m, 6H), 2.03-1.90 (m, 2H). |
| TDH-097 | 1H NMR (400 MHz, Chloroform-d) δ 8.91-8.86 (m, 1H), 8.52-8.50 (m, 1H), 8.00-7.99 (m, |
| | 1H), 7.87-7.86 (m, 1H), 7.65-7.62 (m, 2H), 7.54-7.50 (m, 2H), 7.46-7.37 (m, 3H), 7.04- |
| | 6.98 (m, 2H), 6.43 (s, 1H), 6.32 (s, 2H), 4.80-4.75 (m, 1H), 4.42-4.36 (m, 1H), 3.99-3.96 |
| | (m, 1H), 3.29-3.21 (m, 1H), 2.80-2.72 (m, 1H), 2.28-2.19 (m, 2H), 2.15-2.12 (m, 3H), |
| | 2.02-1.93 (m, 2H); LC/MS (ESI) m/z 558 [M + H]+ |
| TDH-098 | 1H NMR (400 MHz, CDCl3) 0 9.18-9.05 (m, 1H), 8.47-8.36 (m, 2H), 7.90 (d, J = 6.9 Hz, |
| | 2H), 7.62-7.52 (m, 2H), 7.51-7.31 (m, 5H), 7.03-6.92 (m, 2H), 6.52 (s, 2H), 5.83-5.70 |
| | (m, 1H), 4.43-4.17 (m, 3H), 3.01-2.84 (m, 2H), 2.23-2.09 (m, 2H), 2.05-1.90 (m, 2H), |
| | 1.49 (s, 9H); LC/MS (ESI) m/z 615 [M + H]+ |
| TDH-099 | 1H NMR (300 MHz, DMSO-d6) δ 10.66 (s, 1H), 9.09 (d, J = 7.8 Hz, 1H), 8.67 (s, 1H), 8.32 |
| | (s, 1H), 8.05 (s, 1H), 7.70-7.53 (m, 4H), 7.52-7.31 (m, 5H), 7.23-7.10 (m, 2H), 5.81 (d, J = |
| | 7.8 Hz, 1H), 4.55-4.41 (m, 1H), 2.99-2.87 (m, 2H), 2.21-2.11 (m, 2H), 2.11-1.98 (m, |
| | 2H). LC/MS (ESI) m/z 515 [M + H]+ |
| TDH-100 | ¹H NMR (300 MHz, CDCl₃) δ 8.94 (s, 1H), 8.54 (s, 1H), 8.02-7.92 (m, 2H), 7.70-7.62 (m, |
| | 2H), 7.62-7.54 (m, 1H), 7.50-7.38 (m, 2H), 7.17-7.02 (m, 2H), 6.44 (s, 1H), 6.33 (s, 2H), |
| | 5.09-5.01 (m, 1H), 3.83-3.51 (m, 13H), 3.40-3.28 (m, 3H), 1.73-1.57 (m, 4H), 1.45 (s, 9H). |
| TDH-101 | ¹H NMR (300 MHz, CDCl₃) δ 9.37 (s, 1H), 8.52 (s, 1H), 8.01-7.93 (m, 2H), 7.70-7.63 (m, |
| | 2H), 7.62-7.51 (m, 3H), 7.46-7.37 (m, 3H), 7.27-7.16 (m, 1H), 7.08-6.94 (m, 2H), 6.89- |
| | 6.78 (m, 1H), 6.73-6.65 (m, 2H), 6.63 (s, 2H), 6.45 (s, 1H), 4.98-4.84 (m, 1H), 4.32-4.16 |
| | (m, 1H), 4.01-3.94 (m, 2H), 3.31-3.19 (m, 2H), 3.15-3.04 (m, 2H), 2.95-2.69 (m, 4H), |
| | 2.49-2.39 (m, 2H), 2.26-2.14 (m, 6H), 1.59-1.41 (m, 6H). |
| TDH-102 | ¹H NMR (300 MHz, CDCl₃) δ 9.05 (s, 1H), 8.51 (s, 1H), 8.01-7.93 (m, 2H), 7.65-7.60 (m, |
| | 2H), 7.60-7.51 (m, 2H), 7.49-7.36 (m, 4H), 7.18-7.11 (m, 1H), 7.11-6.96 (m, 3H), 6.84- |
| | 6.76 (m, 1H), 6.75-6.68 (m, 1H), 6.55 (s, 2H), 6.42 (s, 1H), 4.94-4.82 (m, 1H), 4.29-4.17 |
| | (m, 1H), 4.01-3.93 (m, 2H), 3.70-3.57 (m, 4H), 3.57-3.40 (m, 10H), 3.25-3.08 (m, 3H), |
| | 2.87-2.59 (m, 7H), 2.21-2.14 (m, 3H). |
| TDH-103 | ¹H NMR (300 MHz, CDCl₃) δ 9.09 (s, 1H), 8.53 (s, 1H), 7.98 (s, 2H), 7.67-7.61 (m, 2H), |
| | 7.59-7.48 (m, 3H), 7.47-7.37 (m, 3H), 7.21-7.05 (m, 2H), 7.05-6.95 (m, 2H), 6.95-6.87 |
| | (m, 1H), 6.73-6.65 (m, 1H), 6.62 (s, 2H), 6.43 (d, J = 1.3 Hz, 1H), 4.95-4.82 (m, 1H), |
| | 4.34-4.16 (m, 1H), 3.94-3.84 (m, 2H), 3.69-3.45 (m, 13H), 3.31-3.06 (m, 3H), 2.84-2.66 |
| | (m, 6H), 2.29-2.16 (m, 4H). |
| TDH-104 | 1H NMR (300 MHz, Chloroform-d) δ 8.92 (s, 1H), 8.56-8.49 (m, 1H), 8.04-7.98 (m, 1H), |
| | 7.93-7.86 (m, 1H), 7.68-7.62 (m, 2H), 7.59-7.52 (m, 2H), 7.49-7.40 (m, 4H), 7.12-6.97 |
| | (m, 3H), 6.93-6.88 (m, 1H), 6.52-6.43 (m, 2H), 4.27 (s, 2H), 3.76-3.69 (m, 4H), 3.52- |
| | 3.46 (m, 2H), 2.91-2.65 (m, 4H), 2.28-2.21 (m, 2H), 2.15-1.94 (m, 5H), 1.64-1.59 (m, 8H). |
| TDH-105 | ¹H NMR (300 MHz, Chloroform-d) δ 8.87 (s, 1H), 8.52 (s, 1H), 8.00 (s, 1H), 7.96-7.93 (m, |
| | 1H), 7.67-7.59 (m, 3H), 7.59-7.50 (m, 3H), 7.44-7.38 (m, 3H), 7.04-6.96 (m, 3H), 6.79- |
| | 6.74 (m, 1H), 6.46-6.41 (m, 2H), 5.81 (s, 1H), 4.31-4.22 (m, 2H), 4.05-3.97 (m, 1H), |
| | 3.80-3.76 (m, 3H), 3.75-3.69 (m, 3H), 3.40 (q, J = 5.7, 5.2 Hz, 3H), 3.26-3.14 (m, 2H), |
| | 2.31-2.16 (m, 3H), 1.62-1.57 (m, 8H). |
| TDH-106 | 1H NMR (300 MHz, DMSO-d6) δ 10.58 (s, 1H), 8.94 (d, J = 7.8 Hz, 1H), 8.27 (d, J = 2.3 |
| | Hz, 1H), 7.89 (d, J = 2.3 Hz, 1H), 7.63-7.48 (m, 6H), 7.43-7.32 (m, 3H), 7.16 (t, J = 8.9 |
| | Hz, 2H), 5.75 (d, J = 7.8 Hz, 1H). |
| TDH-107 | LC/MS [M + H]⁺ = 892 |
| TDH-108 | ¹H NMR (300 MHz, CDCl₃) δ 9.14 (s, 1H), 9.01 (s, 1H), 8.53 (s, 1H), 7.99-7.91 (m, 2H), |
| | 7.67-7.60 (m, 2H), 7.60-7.51 (m, 3H), 7.46-7.34 (m, 4H), 7.20-7.12 (m, 1H), 7.07-6.97 |
| | (m, 2H), 6.44 (s, 2H), 5.79-5.63 (m, 1H), 5.03-4.90 (m, 1H), 4.32-4.18 (m, 1H), 3.85- |
| | 3.71 (m, 2H), 3.33-3.22 (m, 2H), 3.22-3.03 (m, 2H), 3.03-2.67 (m, 6H), 2.52-2.38 (m, |
| | 2H), 2.38-1.88 (m, 16H), 1.38-1.34 (m, 3H). |
| TDH-109 | ¹H NMR (300 MHz, CDCl₃) δ 9.54 (s, 1H), 8.97 (s, 1H), 8.56 (s, 1H), 8.04-7.90 (m, 2H), |
| | 7.73-7.62 (m, 3H), 7.62-7.52 (m, 2H), 7.48-7.37 (m, 3H), 7.10-6.98 (m, 3H), 6.59 (s, |
| | 2H), 5.58 (s, 1H), 5.01-4.87 (m, 1H), 4.33-4.19 (m, 1H), 4.06-3.93 (m, 2H), 3.35-3.23 |
| | (m, 2H), 3.18-3.00 (m, 4H), 2.96-2.71 (m, 3H), 2.53-2.36 (m, 3H), 2.30-1.68 (m, 16H), |
| | 1.38-1.35 (m, 3H). |
| TDH-110 | ¹H NMR (300 MHz, CDCl₃) δ 9.80 (s, 1H), 9.06 (s, 1H), 8.51 (s, 1H), 8.00-7.94 (m, 2H), |
| | 7.67-7.62 (m, 2H), 7.60-7.49 (m, 3H), 7.47-7.38 (m, 3H), 7.37-7.31 (m, 1H), 7.16-7.10 |
| | (m, 1H), 7.06-6.97 (m, 2H), 6.50 (s, 2H), 6.43 (s, 2H), 5.00-4.89 (m, 1H), 4.31-4.16 (m, |
| | 1H), 3.78-3.63 (m, 10H), 3.61-3.56 (m, 2H), 3.52-3.47 (m, 2H), 3.24-3.13 (m, 2H), 2.97- |
| | 2.70 (m, 8H), 2.37-2.06 (m, 10H). |
| TDH-111 | ¹H NMR (300 MHz, CDCl₃) δ 9.67 (s, 1H), 9.04 (s, 1H), 8.54-8.49 (m, 1H), 8.01-7.96 (m, |
| | 2H), 7.71-7.54 (m, 5H), 7.46-7.37 (m, 3H), 7.17-7.12 (m, 1H), 7.06-6.97 (m, 2H), 6.96- |
| | 6.88 (m, 1H), 6.53 (s, 2H), 6.42 (d, 1H), 4.99-4.88 (m, 1H), 4.31-4.19 (m, 1H), 3.90-3.82 |
| | (m, 2H), 3.73-3.57 (m, 10H), 3.51-3.46 (m, 2H), 3.23-3.14 (m, 2H), 2.92-2.69 (m, 8H), |
| | 2.40-2.09 (m, 10H). |
| TDH-112 | 1H NMR (300 MHz, MeOD) δ 9.00 (d, J = 2.3 Hz, 1H), 8.49 (d, J = 2.3 Hz, 1H), 8.30 (s, |
| | 1H), 8.13 (s, 1H), 8.11 (s, 1H), 8.00 (s, 1H), 7.76-7.70 (m, 2H), 7.63-7.55 (m, 2H), 7.55- |
| | 7.46 (m, 3H), 7.16-6.98 (m, 4H), 6.44 (s, 1H), 4.71-4.57 (m, 1H), 3.81-3.73 (m, 1H), |
| | 3.65-3.54 (m, 3H), 2.43-2.31 (m, 4H). LC/MS (ESI) m/z 515 [M + H]+ |
| TDH-113 | ¹H NMR (300 MHz, CDCl₃) δ 10.04 (s, 1H), 8.97 (s, 1H), 8.52 (s, 1H), 8.02-7.94 (m, 2H), |
| | 7.68-7.59 (m, 2H), 7.59-7.51 (m, 2H), 7.51-7.36 (m, 4H), 7.24-7.19 (m, 1H), 7.07-6.98 |
| | (m, 2H), 6.68-6.61 (m, 1H), 6.45-6.38 (m, 2H), 5.89 (s, 1H), 4.96-4.79 (m, 1H), 4.49- |
| | 4.37 (m, 2H), 4.35-4.18 (m, 2H), 3.44-3.24 (m, 3H), 3.20-3.06 (m, 2H), 2.93-2.60 (m, |
| | 4H), 2.50-2.34 (m, 2H), 2.30-2.06 (m, 7H), 1.64-1.45 (m, 5H), 1.45-1.32 (m, 4H). |
| TDH-114 | ¹H NMR (400 MHz, DMSO) δ 11.07 (s, 1H), 10.69 (s, 1H), 8.70 (s, 1H), 8.29 (s, 1H), 7.92 |
| | (s, 1H), 7.79-7.69 (m, 2H), 7.69-7.60 (m, 2H), 7.59-7.53 (m, 1H), 7.53-7.46 (m, 2H), |
| | 7.46-7.40 (m, 1H), 7.35-7.28 (m, 1H), 7.28-7.12 (m, 3H), 7.04-6.96 (m, 1H), 6.93-6.85 |
| | (m, 1H), 6.25 (s, 1H), 5.10-5.00 (m, 1H), 4.91-4.82 (m, 1H), 4.20 (s, 1H), 3.64-3.54 (m, |
| | 1H), 3.54-3.44 (m, 2H), 3.10-2.82 (m, 6H), 2.71-2.58 (m, 3H), 2.16-1.88 (m, 7H), 1.55- |
| | 1.30 (m, 5H), 1.30-1.13 (m, 5H). |
| TDH-115 | ¹H NMR (300 MHz, CDCl₃) δ 9.78 (s, 1H), 9.00 (s, 1H), 8.49 (s, 1H), 7.99-7.93 (m, 2H), |
| | 7.66-7.61 (m, 2H), 7.57-7.52 (m, 2H), 7.45-7.35 (m, 4H), 7.16-7.08 (m, 1H), 7.05-6.96 |
| | (m, 2H), 6.56-6.51 (m, 1H), 6.41 (s, 2H), 4.95-4.78 (m, 1H), 4.42-4.28 (m, 4H), 4.22 (s, |
| | 1H), 3.71-3.56 (m, 10H), 3.53-3.43 (m, 3H), 3.25-3.10 (m, 2H), 2.81-2.60 (m, 6H), 2.29- |
| | 2.10 (m, 6H). |
| TDH-116 | ¹H NMR (300 MHz, CDCl₃) δ 9.01 (d, J = 10.2 Hz, 1H), 8.55 (s, 1H), 8.10-7.93 (m, 2H), |
| | 7.77-7.64 (m, 2H), 7.64-7.52 (m, 3H), 7.52-7.36 (m, 3H), 7.14-6.93 (m, 3H), 6.88-6.66 |
| | (m, 1H), 6.59-6.39 (m, 2H), 5.70 (s, 1H), 4.90 (s, 2H), 4.39-4.11 (m, 2H), 4.03-3.82 (m, |
| | 2H), 3.76-3.36 (m, 11H), 3.34-3.06 (m, 2H), 2.98-2.54 (m, 6H), 2.43-2.10 (m, 7H). |
| TDH-119 | ¹H NMR (300 MHz, CDCl₃) δ 8.97 (s, 1H), 8.81 (s, 1H), 8.54 (s, 1H), 7.97 (d, J = 10.1 Hz, |
| | 2H), 7.69-7.61 (m, 2H), 7.61-7.49 (m, 3H), 7.46-7.39 (m, 3H), 7.12-7.01 (m, 3H), 6.90 |
| | (d, J = 8.6 Hz, 1H), 6.46-6.35 (m, 3H), 6.28-6.22 (m, 1H), 4.98-4.86 (m, 1H), 4.35-4.19 |
| | (m, 1H), 3.34-3.25 (m, 2H), 3.16-3.07 (m, 2H), 2.95-2.70 (m, 4H), 2.49-2.41 (m, 2H), |
| | 2.30-2.11 (m, 7H), 1.74-1.66 (m, 2H), 1.63-1.55 (m, 2H), 1.51-1.40 (m, 4H). |
| TDH-120 | ¹H NMR (400 MHz, CDCl₃) δ 8.95 (s, 1H), 8.54 (d, J = 3.9 Hz, 1H), 7.98 (d, J = 11.0 Hz, |
| | 2H), 7.68-7.63 (m, 3H), 7.60-7.55 (m, 2H), 7.51-7.37 (m, 4H), 7.10-7.00 (m, 2H), 7.01- |
| | 6.94 (m, 1H), 6.49-6.29 (m, 3H), 4.98-4.89 (m, 1H), 4.37-4.21 (m, 1H), 3.32-3.21 (m, |
| | 2H), 3.21-3.04 (m, 3H), 2.99-2.69 (m, 3H), 2.55-2.38 (m, 2H), 2.28-2.04 (m, 7H), 1.77- |
| | 1.54 (m, 4H), 1.51-1.36 (m, 4H). |
| TDH-121 | ¹H NMR (300 MHz, CDCl₃) δ 8.97 (s, 1H), 8.53 (s, 1H), 8.01-7.90 (m, 3H), 7.70-7.62 (m, |
| | 3H), 7.62-7.54 (m, 2H), 7.54-7.36 (m, 5H), 7.15-6.98 (m, 3H), 6.95-6.89 (m, 1H), 6.56- |
| | 6.48 (m, 1H), 6.48-6.32 (m, 3H), 4.95-4.83 (m, 1H), 4.33-4.20 (m, 1H), 3.77-3.67 (m, |
| | 8H), 3.52-3.44 (m, 2H), 3.25-3.13 (m, 2H), 2.90-2.61 (m, 6H), 2.29-2.08 (m, 7H). |
| TDH-122 | ¹H NMR (400 MHz, CDCl₃) δ 8.96 (s, 1H), 8.60-8.51 (m, 1H), 8.05-7.91 (m, 2H), 7.74- |
| | 7.52 (m, 6H), 7.50-7.37 (m, 4H), 7.10-6.98 (m, 3H), 6.50-6.27 (m, 3H), 4.99-4.84 (m, |
| | 1H), 4.33-4.20 (m, 1H), 3.83-3.64 (m, 9H), 3.47-3.39 (m, 1H), 3.26-3.08 (m, 2H), 3.07- |
| | 2.59 (m, 5H), 2.36-2.08 (m, 8H). |
| TDH-123 | LC/MS (ESI) m/z [M + H]⁺: 942.0 |
| TDH-124 | LC/MS (ESI) m/z [M + H]⁻: 940.0 |
| TDH-125 | ¹H NMR (400 MHz, DMSO) δ 11.07 (s, 1H), 10.66 (s, 0.5H), 10.38 (s, 0.5H), 8.68 (d, J = |
| | 7.7 Hz, 1H), 8.57 (s, 0.5H), 8.28 (s, 0.5H), 8.21 (s, 0.5H), 8.18-8.07 (m, 1H), 7.92 (s, |
| | 0.5H), 7.89-7.81 (m, 1H), 7.77-7.70 (m, 1H), 7.66-7.57 (m, 2H), 7.57-7.44 (m, 2H), |
| | 7.46-7.36 (m, 2H), 7.36-7.29 (m, 1H), 7.29-7.21 (m, 1H), 7.21-7.12 (m, 1H), 6.94 (s, |
| | 1H), 6.87 (d, J = 8.4 Hz, 1H), 6.24 (s, 1H), 5.11-4.99 (m, 1H), 4.17 (s, 1H), 3.91-3.82 (m, |
| | 2H), 3.57-3.48 (m, 2H), 3.48-3.40 (m, 3H), 3.31-3.22 (m, 2H), 3.05-2.81 (m, 3H), 2.71- |
| | 2.54 (m, 3H), 2.23-2.08 (m, 2H), 2.08-1.89 (m, 6H). |
| TDH-126 | ¹H NMR (400 MHz, DMSO) δ 11.08 (s, 1H), 10.67 (s, 0.5H), 10.38 (s, 0.5H), 8.75-8.63 |
| | (m, 1H), 8.58 (s, 0.5H), 8.28 (s, 0.5H), 8.20 (s, 0.5H), 8.18-8.10 (m, 1H), 7.91 (s, 0.5H), |
| | 7.88-7.80 (m, 1H), 7.80-7.66 (m, 1H), 7.66-7.38 (m, 4H), 7.31 (s, 1H), 7.28-7.20 (m, |
| | 1H), 7.19-7.08 (m, 2H), 6.80 (d, J = 8.5 Hz, 1H), 6.24 (s, 1H), 5.12-5.00 (m, 1H), 4.47- |
| | 4.24 (m, 2H), 4.24-4.07 (m, 3H), 3.58-3.50 (m, 2H), 3.50-3.39 (m, 3H), 3.31-3.21 (m, |
| | 2H), 3.06-2.92 (m, 2H), 2.92-2.79 (m, 1H), 2.69-2.53 (m, 4H), 2.27-2.11 (m, 2H), 2.11- |
| | 1.72 (m, 6H). |
| TDH-127 | ¹H NMR (400 MHz, DMSO) δ 11.07 (d, J = 3.8 Hz, 1H), 10.66 (s, 0.5H), 10.38 (s, 0.5H), |
| | 8.73-8.64 (m, 1H), 8.63-8.50 (m, 0.5H), 8.34-8.24 (m, 1H), 8.24-8.15 (m, 1H), 7.95-7.90 |
| | (m, 0.5H), 7.89-7.80 (m, 1H), 7.78-7.70 (m, 1H), 7.67-7.60 (m, 1H), 7.59-7.54 (m, 1H), |
| | 7.54-7.46 (m, 2H), 7.46-7.40 (m, 1H), 7.30 (s, 1H), 7.28-7.21 (m, 1H), 7.16 (t, J = 8.8 Hz, |
| | 1H), 7.10-6.78 (m, 2H), 6.24 (s, 1H), 5.13-4.98 (m, 1H), 4.51-3.99 (m, 4H), 3.73-3.41 |
| | (m, 5H), 3.33-3.15 (m, 2H), 3.07-2.79 (m, 4H), 2.73-2.54 (m, 4H), 2.36-2.08 (m, 2H), |
| | 2.07-1.67 (m, 6H). |
| TDH-128 | ¹H NMR (400 MHz, DMSO) δ 11.08 (s, 1H), 10.63 (s, 0.5H), 10.37 (s, 0.5H), 8.67 (d, J = |
| | 7.3 Hz, 1H), 8.57 (s, 0.5H), 8.27 (s, 0.5H), 8.19 (s, 0.5H), 7.96-7.81 (m, 3H), 7.78-7.70 |
| | (m, 1H), 7.70-7.58 (m, 2H), 7.58-7.39 (m, 3H), 7.39-7.21 (m, 4H), 7.21-7.11 (m, 1H), |
| | 6.24 (s, 1H), 5.13-5.02 (m, 1H), 4.27-4.11 (m, 1H), 3.75-3.64 (m, 2H), 3.58-3.50 (m, |
| | 2H), 3.47-3.40 (m, 2H), 3.28-3.20 (m, 2H), 3.10-2.96 (m, 2H), 2.94-2.80 (m, 3H), 2.70- |
| | 2.55 (m, 4H), 2.43-2.29 (m, 1H), 2.29-2.11 (m, 2H), 2.11-1.87 (m, 6H), 1.87-1.65 (m, 4H). |
| TDH-129 | ¹H NMR (400 MHz, DMSO) δ 11.07 (s, 1H), 10.62 (s, 0.5H), 10.37 (s, 0.5H), 8.71-8.64 |
| | (m, 1H), 8.58 (s, 0.5H), 8.28 (s, 0.5H), 8.21 (s, 0.5H), 7.97-7.81 (m, 3H), 7.80-7.69 (m, |
| | 1H), 7.69-7.57 (m, 2H), 7.52-7.41 (m, 2H), 7.37-7.26 (m, 2H), 7.26-7.20 (m, 2H), 7.20- |
| | 7.12 (m, 1H), 6.24 (s, 1H), 5.12-5.01 (m, 1H), 4.23-4.12 (m, 1H), 4.12-4.00 (m, 2H), 3.53 |
| | (t, J = 5.9 Hz, 2H), 3.41 (t, J = 5.8 Hz, 2H), 3.27-3.16 (m, 2H), 3.05-2.94 (m, 4H), 2.94- |
| | 2.82 (m, 1H), 2.64-2.53 (m, 4H), 2.24-2.11 (m, 2H), 2.11-1.89 (m, 6H), 1.83-1.71 (m, |
| | 2H), 1.70-1.53 (m, 2H). |
| TDH-130 | ¹H NMR (500 MHz, CDCl₃) δ 9.82 (s, 0.5H), 9.04 (s, 0.5H), 8.53 (s, 0.5H), 8.45 (s, 0.5H), |
| | 8.01-7.93 (m, 2H), 7.73-7.58 (m, 3H), 7.57-7.47 (m, 1H), 7.47-7.37 (m, 1H), 7.18-7.05 |
| | (m, 3H), 7.05-6.99 (m, 1H), 6.95-6.87 (m, 1H), 6.59-6.51 (m, 1H), 6.51-6.38 (m, 1H), |
| | 4.98-4.77 (m, 1H), 4.38-4.26 (m, 1H), 3.76-3.68 (m, 4H), 3.55-3.44 (m, 2H), 3.27-3.08 |
| | (m, 2H), 2.89-2.59 (m, 5H), 2.48-2.33 (m, 1H), 2.32-2.20 (m, 2H), 2.20-2.01 (m, 4H). |
| TDH-131 | ¹H NMR (500 MHz, CDCl₃) δ 9.93-9.67 (m, 0.5H), 9.08-8.94 (m, 1H), 8.65 (s, 0.5H), |
| | 8.58-8.52 (m, 0.5H), 8.52-8.39 (m, 0.5H), 8.11-7.90 (m, 3H), 7.75-7.61 (m, 4H), 7.61- |
| | 7.54 (m, 1H), 7.49-7.36 (m, 2H), 7.16-6.99 (m, 4H), 6.83-6.72 (m, 1H), 6.44 (s, 1H), |
| | 4.98-4.83 (m, 1H), 4.37-4.20 (m, 1H), 3.84-3.54 (m, 5H), 3.46-3.37 (m, 2H), 3.30-3.18 |
| | (m, 1H), 3.05-2.64 (m, 5H), 2.42-2.17 (m, 4H), 2.15-2.08 (m, 1H), 2.08-2.00 (m, 1H). |
| TDH-132 | 1H NMR (500 MHz, DMSO) δ 11.09 (s, 1H), 10.71-10.64 (m, 1H), 8.70 (s, 1H), 8.29 (s, |
| | 1H), 8.10-8.03 (m, 1H), 7.93 (s, 1H), 7.78-7.70 (m, 2H), 7.64-7.59 (m, 2H), 7.54-7.47 |
| | (m, 2H), 7.47-7.36 (m, 2H), 7.36-7.28 (m, 2H), 7.21-7.12 (m, 2H), 6.98-6.91 (m, 1H), |
| | 6.91-6.83 (m, 1H), 6.24 (s, 1H), 5.09-5.01 (m, 1H), 4.19 (s, 1H), 3.87-3.80 (m, 2H), |
| | 3.14-3.07 (m, 2H), 3.03-2.83 (m, 3H), 2.67-2.54 (m, 2H), 2.35-2.21 (m, 2H), 2.09-1.92 |
| | (m, 6H), 1.50-1.35 (m, 4H), 1.32-1.21 (m, 5H). |
| TDH-133 | ¹H NMR (500 MHz, DMSO) δ 11.19 (s, 1H), 10.64 (s, 1H), 8.70 (s, 1H), 8.30 (s, 1H), |
| | 8.00-7.90 (m, 2H), 7.87-7.80 (m, 1H), 7.76-7.69 (m, 2H), 7.67-7.59 (m, 2H), 7.52-7.48 |
| | (m, 2H), 7.46-7.37 (m, 2H), 7.34-7.28 (m, 2H), 7.19-7.13 (m, 2H), 6.23 (s, 1H), 5.16- |
| | 5.10 (m, 1H), 4.78 (s, 2H), 4.23-4.14 (m, 1H), 3.20-3.11 (m, 2H), 3.01-2.85 (m, 3H), |
| | 2.66-2.54 (m, 2H), 2.35-2.25 (m, 2H), 2.13-1.90 (m, 7H), 1.51-1.36 (m, 4H), 1.36-1.23 |
| | (m, 4H). |
| TDH-134 | ¹H NMR (500 MHz, DMSO) δ 11.16 (s, 1H), 10.64 (s, 0.5H), 10.39 (s, 0.5H), 8.73-8.65 |
| | (m, 1H), 8.57 (s, 0.5H), 8.27 (s, 0.5H), 8.20 (s, 0.5H), 8.04-7.95 (m, 1H), 7.91 (s, 0.5H), |
| | 7.88-7.79 (m, 2H), 7.75-7.68 (m, 1H), 7.67-7.59 (m, 1H), 7.55-7.46 (m, 3H), 7.46-7.39 |
| | (m, 2H), 7.35-7.29 (m, 1H), 7.29-7.20 (m, 1H), 7.20-7.12 (m, 1H), 6.24 (s, 1H), 5.16- |
| | 5.10 (m, 1H), 4.80 (s, 2H), 4.20-4.09 (m, 1H), 3.59-3.52 (m, 2H), 3.52-3.43 (m, 2H), |
| | 3.06-2.85 (m, 3H), 2.69-2.52 (m, 5H), 2.22-2.10 (m, 2H), 2.10-1.88 (m, 6H). |
| TDH-135 | ¹H NMR (500 MHz, DMSO) δ 11.16 (s, 1H), 10.64 (s, 1H), 8.69 (s, 1H), 8.28 (s, 1H), |
| | 8.06-7.99 (m, 1H), 7.93-7.89 (m, 1H), 7.86-7.79 (m, 1H), 7.78-7.69 (m, 2H), 7.67-7.59 |
| | (m, 2H), 7.54-7.46 (m, 3H), 7.45-7.38 (m, 2H), 7.31 (s, 2H), 7.19-7.14 (m, 1H), 6.24 (s, |
| | 1H), 5.16-5.09 (m, 1H), 4.80 (s, 2H), 4.21-4.09 (m, 1H), 3.59-3.45 (m, 8H), 3.34-3.30 |
| | (m, 2H), 3.04-2.85 (m, 3H), 2.66-2.52 (m, 4H), 2.20-2.09 (m, 2H), 2.09-1.88 (m, 5H). |
| TDH-136 | 1H NMR (400 MHz, Chloroform-d) δ 8.96 (s, 1H), 8.53(s, 1H), 8.48-8.36(m, 1H), 7.97(s, |
| | 1H), 7.94-7.88(m, 1H), 7.69-7.60(m, 2H), 7.60-7.46(m, 3H), 7.46-7.35(m, 3H), 7.26 (s, 1H), |
| | 7.13 (d, J = 7.2 Hz, 1H), 7.03 (t, J = 8.6 Hz, 2H), 6.80 (d, J = 8.5 Hz, 1H), 6.41 (s, 1H), |
| | 6.37 (s, 1H), 4.93 (dd, J = 12.2, 5.4 Hz, 1H), 4.65 (d, J = 13.2 Hz, 1H), 4.25-4.14 (m, 1H), |
| | 4.03 (d, J = 4.4 Hz, 2H), 3.76 (d, J = 12.9 Hz, 1H), 3.15-2.94 (m, 3H), 2.94-2.80 (m, 2H), |
| | 2.77-2.64 (m, 2H), 2.26 (d, J = 6.6 Hz, 2H), 2.25-2.05 (m, 5H), 2.04 (s, 2H), 1.93 (d, J = |
| | 12.8 Hz, 1H), 1.82 (d, J = 12.9 Hz, 2H) 1.16 (m, J = 13.7 Hz, 2H). |
| TDH-137 | 1H NMR (400 MHz, Chloroform-d) δ 9.13-9.02 (m, 1H), 8.54 (d, J = 4.8 Hz, 2H), 7.98 (d, |
| | J = 4.8 Hz, 1H), 7.94 (d, J = 4.8 Hz, 1H), 7.64 (dd, J = 7.9, 4.6 Hz, 3H), 7.58 (dt, J = 9.7, |
| | 5.0 Hz, 2H), 7.42 (p, J = 7.9, 7.1 Hz, 3H), 7.27 (d, J = 4.9 Hz, 2H), 7.04 (td, J = 8.4, 4.2 |
| | Hz, 2H), 6.97 (d, J = 3.5 Hz, 1H), 6.88-6.80 (m, 1H), 6.42 (t, J = 7.2 Hz, 2H), 5.95 (d, J = |
| | 4.8 Hz, 1H), 4.97-4.91 (m, 1H), 4.65 (d, J = 13.4 Hz, 1H), 4.27-4.16(m, 1H), 4.01-3.92 (m, |
| | 2H), 3.84-3.72 (m, 1H), 3.12 (d, J = 13.2 Hz, 1H), 3.02 (s, 2H), 2.86-2.80 (m, 2H), 2.79- |
| | 2.66 (m, 2H), 2.27 (d, J = 5.5 Hz, 2H), 2.23-2.18 (m, 8.2 Hz, 5H), 2.15-1.90 (m, 2H), 2.08- |
| | 1.92 (m, 3H), 1.29-1.19 (m, 2H). |
| TDH-138 | 1H NMR (400 MHz, Chloroform-d) δ 8.98 (s, 1H), 8.55 (s, 1H), 8.28-8.09 (m, 1H), 8.00 (s, |
| | 1H), 7.98-7.90(m, 1H), 7.65 (d, J = 7.5 Hz, 2H), 7.56-7.54 (m, 2H), 7.47-7.39 (m, 3H), |
| | 7.22 (d, J = 7.0 Hz, 1H), 7.06 (t, J = 8.4 Hz, 2H), 6.70 (d, J = 8.5 Hz, 1H), 6.44 (s, 1H), |
| | 6.36 (s, 2H), 5.00-4.84 (m, 1H), 4.69-4.58 (m, 1H), 4.57-4.41 (m, 3H), 4.27-4.14 (m, 1H), |
| | 3.73 (t, J = 7.8 Hz, 1H), 3.65-3.55(m, 1H), 3.15-2.95 (m, 3H), 2.95-2.57 (m, 5H), 2.32- |
| | 1.96 (m, 11H), 1.9-1.46 (m, 12H), 1.17-1.09 (m, 2H). |
| TDH-139 | ¹H NMR (500 MHz, CDCl₃) δ 9.70 (s, 1H), 9.03 (s, 1H), 8.54 (s, 1H), 8.03-7.93 (m, 2H), |
| | 7.69-7.64 (m, 2H), 7.63-7.54 (m, 3H), 7.47-7.36 (m, 4H), 7.19-7.14 (m, 1H), 7.08-7.00 |
| | (m, 2H), 6.47-6.37 (m, 3H), 4.99-4.88 (m, 1H), 4.25 (s, 1H), 3.84-3.78 (m, 1H), 3.78- |
| | 3.73 (m, 1H), 3.72-3.63 (m, 10H), 3.62-3.55 (m, 2H), 3.55-3.42 (m, 2H), 3.27-3.13 (m, |
| | 2H), 3.01-2.63 (m, 8H), 2.38-2.14 (m, 6H), 2.03-1.94 (m, 6H). |
| TDH-140 | ¹H NMR (500 MHz, CDCl₃) δ 9.08-8.97 (m, 2H), 8.54 (s, 1H), 7.99 (s, 1H), 7.95 (s, 1H), |
| | 7.71-7.64 (m, 3H), 7.61-7.55 (m, 2H), 7.49-7.38 (m, 3H), 7.28-7.24 (m, 1H), 7.09-7.00 |
| | (m, 3H), 6.58-6.39 (m, 3H), 4.98-4.90 (m, 1H), 4.29-4.19 (m, 1H), 3.99-3.90 (m, 2H), |
| | 3.76-3.62 (m, 9H), 3.62-3.54 (m, 2H), 3.53-3.44 (m, 2H), 3.21-3.06 (m, 2H), 3.06-2.94 |
| | (m, 2H), 2.94-2.79 (m, 2H), 2.79-2.65 (m, 3H), 2.44-2.33 (m, 1H), 2.33-2.08 (m, 6H), |
| | 1.95-1.85 (m, 6H). |
| TDH-141 | ¹H NMR (500 MHz, DMSO) δ 11.13-11.05 (m, 1H), 10.62 (s, 1H), 8.69 (s, 1H), 8.29 (s, |
| | 1H), 8.16-8.09 (m, 1H), 7.95-7.90 (m, 1H), 7.75-7.71 (m, 2H), 7.65-7.60 (m, 2H), 7.59- |
| | 7.54 (m, 1H), 7.52-7.47 (m, 2H), 7.46-7.41 (m, 1H), 7.29 (s, 2H), 7.18-7.12 (m, 2H), |
| | 6.82-6.77 (m, 1H), 6.24 (s, 1H), 5.10-5.01 (m, 1H), 4.38-4.28 (m, 1H), 4.18-4.11 (m, |
| | 2H), 3.58-3.42 (m, 13H), 3.28-3.22 (m, 2H), 3.06-2.93 (m, 2H), 2.93-2.81 (m, 1H), 2.67- |
| | 2.51 (m, 6H), 2.22-2.10 (m, 2H), 2.07-1.89 (m, 6H). |
| TDH-142 | ¹H NMR (500 MHz, DMSO) δ 11.06 (s, 1H), 10.71 (s, 1H), 8.69 (s, 1H), 8.29 (s, 1H), |
| | 8.01-7.94 (m, 1H), 7.92 (s, 1H), 7.77-7.73 (m, 1H), 7.68-7.61 (m, 2H), 7.59-7.54 (m, |
| | 1H), 7.52-7.46 (m, 2H), 7.46-7.40 (m, 1H), 7.29 (s, 2H), 7.22-7.12 (m, 2H), 7.03-6.97 |
| | (m, 1H), 6.91-6.84 (m, 1H), 6.26 (s, 1H), 5.09-4.99 (m, 1H), 4.90-4.78 (m, 1H), 4.23- |
| | 4.11 (m, 1H), 3.63-3.56 (m, 1H), 3.56-3.43 (m, 9H), 3.40-3.35 (m, 3H), 3.27-3.13 (m, |
| | 2H), 3.02-2.94 (m, 2H), 2.94-2.82 (m, 1H), 2.72-2.63 (m, 1H), 2.63-2.51 (m, 6H), 2.22- |
| | 2.10 (m, 2H), 2.10-1.81 (m, 5H). |
| TDH-143 | ¹H NMR (500 MHz, CDCl₃) δ 10.11 (s, 1H), 9.08 (s, 1H), 8.52 (s, 1H), 7.97 (d, J = 7.5 Hz, |
| | 2H), 7.72-7.62 (m, 2H), 7.62-7.54 (m, 2H), 7.54-7.46 (m, 1H), 7.45-7.38 (m, 2H), 7.25- |
| | 7.19 (m, 1H), 7.19-7.12 (m, 1H), 7.07-6.93 (m, 2H), 6.84-6.69 (m, 2H), 6.56 (s, 2H), 6.43 |
| | (s, 1H), 4.94-4.82 (m, 1H), 4.27-4.17 (m, 1H), 4.03-3.94 (m, 2H), 3.73-3.59 (m, 6H), |
| | 3.59-3.35 (m, 8H), 3.26-3.12 (m, 2H), 2.89-2.62 (m, 5H), 2.37-1.97 (m, 7H). |
| TDH-144 | ¹H NMR (500 MHz, CDCl3) δ 9.42 (s, 1H), 9.07 (s, 1H), 8.52 (s, 1H), 7.95 (s, 2H), 7.73- |
| | 7.63 (m, 2H), 7.63-7.54 (m, 3H), 7.48-7.36 (m, 3H), 7.10-6.98 (m, 2H), 6.98-6.89 (m, |
| | 1H), 6.79-6.70 (m, 1H), 6.64-6.34 (m, 3H), 5.91 (s, 1H), 4.94-4.83 (m, 1H), 4.31-4.15 |
| | (m, 1H), 3.96-3.86 (m, 2H), 3.74-3.64 (m, 5H), 3.63-3.53 (m, 5H), 3.53-3.42 (m, 2H), |
| | 3.25-3.04 (m, 2H), 2.91-2.61 (m, 5H), 2.36-1.94 (m, 8H). |
| TDH-145 | 1H NMR (500 MHz, Chloroform-d) δ 8.97 (s, 1H), 8.61-8.55 (m, 1H), 8.0-7.90 (m, 2H), |
| | 7.70-7.65 (m, 3H), 7.59 (s, 2H), 7.47-7.38 (m, 3H), 7.12-6.98 (m, 3H), 6.81 (d, J = 8.4 Hz, |
| | 1H), 6.62 (m, 1H), 6.55-6.39 (m, 2H), 5.0-4.81 (m, 2H), 4.69-4.58 (m, 1H), 4.23 (s, 1H), |
| | 3.96-3.76 (m, 4H), 3.75-3.59 (m, 4H), 3.12 (q, J = 7.5 Hz, 2H), 3.07-2.97 (m, 2H), 2.92- |
| | 2.82 (m, 3H), 2.81-2.69 (m, 2H), 2.69-2.60 (m, 2H), 2.21-2.09(m, 5H), 2.09-1.95 (m, 6H), |
| | 1.77 (s, 4H), 1.64 (s, 8H), 1.60-1.51 (m, 10H)1.47 (s, 7H), 1.13-1.07 (m, 2H). |
| TDH-146 | 1H NMR (500 MHz, Chloroform-d) δ 9.00 (s, 1H), 8.68 (s, 1H), 8.55 (s, 1H), 7.99 (s, 1H), |
| | 7.94 (s, 1H), 7.65 (d, J = 7.5 Hz, 2H), 7.62-7.58 (m, 3H), 7.43-7.40 (m, 3H), 7.39 (t, J = |
| | 7.0 Hz, 2H), 7.20 (d, J = 8.5 Hz, 1H), 7.04 (t, J = 8.5 Hz, 2H), 6.44 (s, 3H), 5.02-4.93 (m, |
| | 1H), 4.67-4.55 (m, 1H), 4.21 (t, J = 11.8 Hz, 1H), 3.98-3.85 (m, 1H), 3.90-3.75 (m, 3H), |
| | 3.025-3.01 (m, 7H), 2.95-2.68 (m, 6H), 2.64-2.52 (m, 1H), 2.30-2.24 (m, 3H), 2.24-2.16 |
| | (m, 5H), 2.16-1.99 (m, 9H), 1.98-1.75 (m, 5H). |
| TDH-147 | 1H NMR (500 MHz, Chloroform-d) δ 8.98 (s, 1H), 8.55 (s, 1H), 8.37 (s, 1H), 8.00 (s, 1H), |
| | 7.95 (s, 1H), 7.70 (d, J = 8.5 Hz, 1H), 7.65 (d, J = 7.5 Hz, 2H), 7.62-7.55 (m, 2H), 7.48- |
| | 7.37 (m, 3H), 7.30 (s, 1H), 7.06 (q, J = 8.4 Hz, 3H), 6.44 (s, 1H), 6.39 (s, 2H), 4.98- |
| | 4.94(m, 1H), 4.70-4.63 (m, 1H), 4.26-4.17 (m, 1H), 4.04-3.91 (m, 3H), 3.16-2.97 (m, 5H), |
| | 2.96-2.68 (m, 5H), 2.61 (t, J = 12.8 Hz, 1H), 2.35-2.25 (m, 2H), 2.2417-2.11 (m, 6H), |
| | 2.10-1.92(m, 6H), 1.91-1.74 (m, 5H) |
| TDH-148 | 1H NMR (500 MHz, Chloroform-d) δ 9.06-8.87 (m, 1H), 8.55 (s, 1H), 8.07-7.97 (m, 1H), |
| | 7.97-7.89 (m, 1H), 7.74-7.64 (m, 3H), 7.61-7.49 (m, 3H), 7.48-7.38 (m, 3H), 7.37-7.11 (m, |
| | 1H), 7.10-6.99(m, 2H), 6.26-6.50 (m, 3H), 5.04-4.91 (m, 2H), 4.60-4.50 (m, 1H), 4.20 (s, |
| | 1H), 4.05-4.0 (m, 1H), 3.18-2.93 (m, 4H), 2.95-2.60 (m, 4H), 2.32-2.24 (m, 3H), 2.23-2.07 |
| | (m, 7H), 2.08-1.97 (m., 2H), 1.97-1.70 (m, 5H). |
| TDH-149 | 1H NMR (400 MHz, Chloroform-d) δ 9.01 (s, 1H), 8.55 (s, 1H), 8.33 (s, 1H), 8.00 (s, 1H), |
| | 7.95 (s, 1H), 7.70-7.63 (m, 2H), 7.63-7.53 (m, 3H), 7.48-7.36 (m, 4H), 7.21 (d, J = 8.4 Hz, |
| | 1H), 7.10-7.01 (m, 2H), 6.44 (s, 1H), 6.38 (s, 2H), 5.02-4.95 (m, 1H), 4.27-4.17 (m, 1H), |
| | 3.83-3.73 (m, 2H), 3.11-3.00 (m, 2H), 2.97-2.90 (m, 3H), 2.89-2.72 (m, 3H), 2.37-2.29 (m, |
| | 2H), 2.25-2.17 (m, 4H), 2.17-2.04 (m, 4H), 1.98-1.91 (m, 2H), 1.54-1.48 (m, 2H). |
| TDH-150 | 1H NMR (500 MHz, Chloroform-d) δ 8.99 (s, 1H), 8.56 (s, 1H), 8.00 (s, 1H), 7.95 (s, 1H), |
| | 7.70 (d, J = 8.5 Hz, 1H), 7.66 (d, J = 7.4 Hz, 2H), 7.62-7.59 (m, 2H), 7.47-7.39 (m, 4H), |
| | 7.32-7.30(m, 1H), 7.10-7.02 (m, 3H), 6.44 (s, 3H), 5.01-4.92 (m, 1H), 4.22 (s, 1H), 4.03- |
| | 3.94(m 2H), 3.03 (s, 2H), 3.02-2.91 (m, 3H), 2.91-2.74 (m, 3H), 2.36-2.26(m, 2H), 2.25- |
| | 2.12 (m, 7H), 2.14-2.25 (m, 4H), 1.96-1.90 (m, 2H), 1.34-1.30 (m, 2H). |
| TDH-151 | 1H NMR (500 MHz, Chloroform-d) δ 9.00 (s, 1H), 8.73-8.65 (m, 1H), 8.55 (s, 1H), 8.03- |
| | 7.99 (m, 2H), 7.95 (s, 1H), 7.65 (d, J = 7.4 Hz, 2H), 7.62-7.56(m, 2H), 7.54 (s, 1H), 7.50- |
| | 7.39 (m, 4H), 7.05 (t, J = 8.4 Hz, 2H), 6.43 (s, 3H), 5.82-5.75 (m, 1H), 4.71-4.61 (m, 1H), |
| | 4.28-4.17 (m, 1H), 4.13-4.03 (m, 2H), 4.02-3.92 (m, 1H), 3.18-3.08 (m, 2H), 3.08-2.89 (m, |
| | 5H), 2.88-2.78 (m, 2H), 2.65-2.55 (m, 1H), 2.43-2.36 (m, 1H), 2.31-2.24 (m, 2H), 2.24- |
| | 2.11 (m, 5H), 2.10-1.93 (m, 6H), 1.91-7.86(m, 3H), 1.83 (s, 1H), 1.19-1.10 (m, 2H). |
| TDH-152 | 1H NMR (500 MHz, Chloroform-d) δ 9.00 (s, 1H), 8.65-8.50 (m, 2H), 8.03-7.91 (m, 3H), |
| | 7.70-7.62 (m, 2H), 7.61-7.54 (m, 2H), 7.49-7.36 (m, 3H), 7.26 (s, 1H), 7.17 (s, 1H), 7.12- |
| | 7.01 (m, 2H), 6.52-6.29 (m, 3H), 6.06 (s, 1H), 5.85-5.75 (m, 1H), 4.71-4.62 (m, 1H), 4.26- |
| | 4.17 (m, 1H), 4.02 (s, 2H), 3.87-3.77 (m, 1H), 3.16-3.07 (m, 1H), 3.08-2.92 (m, 4H), 2.92- |
| | 2.80(m, 1H), 2.79-2.70 (m, 1H), 2.46-2.37 (m, 1H), 2.35-2.25 (m, 2H), 2.24-2.13 (m, 4H), |
| | 2.12-2.029 (m, 3H) 1.94-1.84 (m, 2H), 1.23-1.14 (m, 2H). |
| TDH-153 | ¹H NMR (500 MHz, CDCl₃) δ 9.13-9.01 (m, 1H), 8.54 (s, 1H), 8.02-7.93 (m, 2H), 7.69- |
| | 7.64 (m, 2H), 7.62-7.55 (m, 2H), 7.53-7.47 (m, 1H), 7.46-7.34 (m, 4H), 7.14-7.09 (m, |
| | 1H), 7.09-6.97 (m, 2H), 6.97-6.88 (m, 1H), 6.57-6.30 (m, 4H), 4.96-4.82 (m, 1H), 4.32- |
| | 4.18 (m, 1H), 3.76-3.57 (m, 14H), 3.52-3.40 (m, 2H), 3.27-3.13 (m, 2H), 2.90-2.61 (m, |
| | 5H), 2.18-2.02 (m, 5H). |
| TDH-154 | ¹H NMR (500 MHz, CDCl₃) δ 9.12 (s, 0.5H), 9.04 (s, 1H), 8.74-8.51 (m, 0.5H), 8.47 (s, |
| | 1H), 7.98 (s, 1H), 7.96-7.90 (m, 1H), 7.68-7.63 (m, 2H), 7.63-7.54 (m, 3H), 7.48-7.38 |
| | (m, 4H), 7.09-7.01 (m, 3H), 6.84-6.77 (m, 1H), 6.54-6.30 (m, 3H), 4.89-4.81 (m, 1H), |
| | 4.26 (s, 1H), 3.74-3.64 (m, 14H), 3.46-3.37 (m, 2H), 3.21-3.09 (m, 2H), 2.88-2.54 (m, |
| | 5H), 2.12-1.99 (m, 5H). |
| TDH-155 | ¹H NMR (500 MHz, CDCl₃) δ 9.21-9.14 (m, 1H), 8.56 (s, 1H), 8.03-7.95 (m, 2H), 7.78- |
| | 7.62 (m, 4H), 7.62-7.56 (m, 2H), 7.54-7.48 (m, 1H), 7.46-7.36 (m, 3H), 7.20-7.14 (m, |
| | 1H), 7.06-6.96 (m, 2H), 6.55-6.33 (m, 3H), 5.01-4.89 (m, 1H), 4.64 (s, 2H), 4.29-4.17 |
| | (m, 1H), 3.76-3.51 (m, 16H), 3.29-3.17 (m, 2H), 2.92-2.62 (m, 6H), 2.18-2.07 (m, 4H). |
| TDH-156 | ¹H NMR (500 MHz, CDCl₃) δ 9.27 (s, 1H), 9.05 (s, 1H), 8.53 (s, 1H), 7.97 (d, J = 11.6 Hz, |
| | 2H), 7.68-7.63 (m, 2H), 7.63-7.54 (m, 3H), 7.48-7.37 (m, 4H), 7.18 (d, J = 8.4 Hz, 1H), |
| | 7.03 (t, J = 8.4 Hz, 2H), 6.60-6.37 (m, 3H), 5.73-5.63 (m, 1H), 5.02-4.93 (m, 1H), 4.34- |
| | 4.20 (m, 1H), 3.85-3.75 (m, 2H), 3.33-3.23 (m, 2H), 3.23-3.10 (m, 2H), 3.04-2.92 (m, |
| | 2H), 2.90-2.69 (m, 3H), 2.56-2.40 (m, 2H), 2.38-2.29 (m, 2H), 2.28-2.19 (m, 3H), 2.19- |
| | 2.08 (m, 4H), 2.08-1.92 (m, 6H), 1.54-1.47 (m, 3H), 1.34-1.30 (m, 10H). |
| TDH-157 | ¹H NMR (500 MHz, CDCl₃) δ 9.02 (s, 1H), 8.88 (s, 1H), 8.55 (s, 1H), 7.98 (d, J = 15.3 Hz, |
| | 2H), 7.76-7.63 (m, 3H), 7.63-7.51 (m, 2H), 7.49-7.37 (m, 3H), 7.11-6.97 (m, 3H), 6.64- |
| | 6.32 (m, 3H), 5.57 (s, 1H), 4.98-4.91 (m, 1H), 4.34-4.20 (m, 1H), 4.03-3.95 (m, 2H), |
| | 3.32-3.25 (m, 2H), 3.22-3.11 (m, 2H), 3.09-3.01 (m, 2H), 2.94-2.66 (m, 4H), 2.59-2.32 |
| | (m, 4H), 2.26-2.11 (m, 6H), 2.00-1.95 (m, 2H), 1.90-1.81 (m, 4H), 1.54-1.48 (m, 3H), |
| | 1.35-1.31 (m, 10H). |
| TDH-158 | ¹H NMR (500 MHz, DMSO) δ 11.09 (s, 1H), 10.64 (s, 1H), 8.70 (s, 1H), 8.65 (s, 0.5H), |
| | 8.29 (s, 1H), 8.27 (s, 0.5H), 8.05-7.98 (m, 1H), 7.92 (s, 1H), 7.74-7.72 (m, 1H), 7.65-7.56 |
| | (m, 3H), 7.52-7.47 (m, 2H), 7.47-7.40 (m, 1H), 7.33-7.27 (m, 2H), 7.19-7.12 (m, 3H), |
| | 6.84-6.79 (m, 1H), 6.24 (s, 1H), 5.08-5.02 (m, 1H), 4.37-4.31 (m, 2H), 4.17-4.13 (m, |
| | 2H), 3.10-3.04 (m, 2H), 2.94-2.83 (m, 2H), 2.66-2.55 (m, 2H), 2.07-2.00 (m, 4H), 1.97- |
| | 1.91 (m, 2H), 1.52-1.36 (m, 6H), 1.30-1.24 (m, 16H). |
| TDH-159 | ¹H NMR (500 MHz, DMSO) δ 11.08 (s, 1H), 10.66 (s, 1H), 8.74-8.64 (m, 1H), 8.29 (s, |
| | 1H), 7.94 (s, 1H), 7.90-7.82 (m, 1H), 7.76-7.72 (m, 2H), 7.65-7.59 (m, 2H), 7.58-7.53 |
| | (m, 1H), 7.53-7.46 (m, 2H), 7.46-7.40 (m, 1H), 7.35-7.27 (m, 2H), 7.20-7.14 (m, 2H), |
| | 6.98 (s, 1H), 6.89-6.84 (m, 1H), 6.24 (s, 1H), 5.09-5.00 (m, 1H), 4.89-4.82 (m, 1H), |
| | 3.62-3.55 (m, 1H), 3.52-3.43 (m, 1H), 3.10-2.95 (m, 3H), 2.93-2.84 (m, 1H), 2.68-2.53 |
| | (m, 3H), 2.10-1.96 (m, 6H), 1.52-1.41 (m, 2H), 1.38-1.31 (m, 2H), 1.27-1.16 (m, 18H). |
| TDH-160 | ¹H NMR (500 MHz, DMSO) δ 11.08 (s, 1H), 10.64 (s, 1H), 8.70 (s, 1H), 8.65 (s, 0.5H), |
| | 8.29 (s, 1H), 8.27 (s, 0.5H), 7.92 (s, 1H), 7.74-7.72 (m, 1H), 7.65-7.59 (m, 2H), 7.59-7.54 |
| | (m, 1H), 7.54-7.47 (m, 2H), 7.46-7.41 (m, 1H), 7.32-7.30 (m, 1H), 7.29-7.26 (m, 1H), |
| | 7.20-7.14 (m, 2H), 7.14-7.10 (m, 1H), 6.96-6.92 (m, 1H), 6.87 6.82 (m, 1H), 6.24 (s, 1H), |
| | 5.06-5.01 (m, 1H), 4.24-4.14 (m, 2H), 3.19-3.12 (m, 2H), 3.00-2.92 (m, 3H), 2.90-2.84 |
| | (m, 1H), 2.67-2.52 (m, 2H), 2.06-2.01 (m, 4H), 1.98-1.94 (m, 2H), 1.59-1.54 (m, 2H), |
| | 1.49-1.41 (m, 4H), 1.31-1.24 (m, 16H). |
| TDH-161 | ¹H NMR (500 MHz, CDCl₃) δ 9.08 (s, 1H), 8.54 (s, 1H), 8.00-7.93 (m, 2H), 7.69-7.64 (m, |
| | 2H), 7.60-7.52 (m, 3H), 7.47-7.37 (m, 3H), 7.26-7.21 (m, 1H), 7.06-7.00 (m, 2H), 6.86- |
| | 6.80 (m, 1H), 6.72-6.66 (m, 1H), 6.60-6.47 (m, 3H), 6.45 (s, 1H), 4.97-4.90 (m, 1H), |
| | 4.30-4.21 (m, 1H), 3.99-3.94 (m, 2H), 3.34-3.19 (m, 3H), 3.17-3.08 (m, 2H), 2.94-2.68 |
| | (m, 4H), 2.49-2.39 (m, 2H), 2.26-2.16 (m, 4H), 2.16-2.10 (m, 4H), 1.58-1.52 (m, 2H), |
| | 1.49-1.44 (m, 2H), 1.36-1.26 (m, 10H). |
| TDH-162 | ¹H NMR (500 MHz, CDCl₃) δ 9.10 (s, 1H), 8.55 (s, 1H), 8.02-7.93 (m, 2H), 7.70-7.61 (m, |
| | 3H), 7.61-7.53 (m, 2H), 7.49-7.37 (m, 3H), 7.09-6.95 (m, 3H), 6.86-6.77 (m, 1H), 6.62- |
| | 6.41 (m, 3H), 6.31 (s, 1H), 5.44 (s, 1H), 4.96-4.87 (m, 1H), 4.36-4.18 (m, 1H), 3.91-3.85 |
| | (m, 2H), 3.36-3.25 (m, 2H), 3.21-3.10 (m, 2H), 2.92-2.63 (m, 4H), 2.52-2.40 (m, 2H), |
| | 2.27-2.06 (m, 8H), 1.54-1.47 (m, 4H), 1.31-1.27 (m, 10H). |
| TDH-163 | ¹H NMR (500 MHz, CDCl₃) δ 9.06 (s, 1H), 8.56 (s, 1H), 8.03-7.95 (m, 2H), 7.79-7.72 (m, |
| | 1H), 7.72-7.62 (m, 2H), 7.61-7.51 (m, 3H), 7.48-7.36 (m, 5H), 7.23-7.18 (m, 1H), 7.07- |
| | 7.00 (m, 2H), 6.56-6.29 (m, 3H), 5.02-4.93 (m, 1H), 4.70-4.61 (m, 2H), 4.37-4.23 (m, |
| | 1H), 3.50-3.32 (m, 3H), 3.25-3.14 (m, 2H), 2.96-2.81 (m, 3H), 2.81-2.67 (m, 1H), 2.54- |
| | 2.45 (m, 2H), 2.28-2.12 (m, 8H), 1.66-1.57 (m, 4H), 1.44-1.31 (m, 10H). |
| TDH-164 | ¹H NMR (500 MHz, CDCl₃) δ 9.43 (s, 1H), 8.99 (s, 1H), 8.58-8.52 (m, 1H), 8.02-7.94 (m, |
| | 2H), 7.90-7.84 (m, 1H), 7.70-7.62 (m, 2H), 7.61-7.53 (m, 2H), 7.48-7.38 (m, 4H), 7.28- |
| | 7.24 (m, 1H), 7.09-6.99 (m, 2H), 6.52 (s, 2H), 6.47-6.43 (m, 1H), 5.01-4.91 (m, 1H), 4.64 |
| | (s, 2H), 4.28 (s, 1H), 3.43-3.36 (m, 2H), 3.23-3.10 (m, 2H), 2.95-2.71 (m, 3H), 2.53-2.40 |
| | (m, 2H), 2.27-2.12 (m, 6H), 2.08-2.01 (m, 2H), 1.66-1.49 (m, 6H), 1.35-1.31 (m, 10H). |
| TDH-165 | ¹H NMR (500 MHz, CDCl₃) δ 10.34 (s, 1H), 9.05 (s, 1H), 8.55 (s, 1H), 8.04-7.93 (m, 2H), |
| | 7.86-7.80 (m, 1H), 7.70-7.62 (m, 2H), 7.62-7.54 (m, 2H), 7.46-7.36 (m, 4H), 7.27-7.20 |
| | (m, 1H), 7.06-6.98 (m, 2H), 6.67-6.59 (m, 1H), 6.57-6.49 (m, 1H), 6.43 (s, 1H), 5.03- |
| | 4.94 (m, 1H), 4.66 (s, 2H), 4.30-4.19 (m, 1H), 3.43-3.28 (m, 2H), 3.16-3.05 (m, 2H), |
| | 2.93-2.71 (m, 3H), 2.49-2.39 (m, 2H), 2.26-2.01 (m, 8H), 1.60-1.50 (m, 4H), 1.41-1.34 |
| | (m, 4H). |
| TDH-166 | ¹H NMR (500 MHz, CDCl₃) δ 9.47 (s, 1H), 9.02 (s, 1H), 8.55 (s, 1H), 8.03-7.93 (m, 2H), |
| | 7.91-7.80 (m, 1H), 7.70-7.64 (m, 2H), 7.64-7.56 (m, 2H), 7.46-7.40 (m, 3H), 7.28-7.25 |
| | (m, 1H), 7.22 (s, 1H), 7.07-6.99 (m, 2H), 6.63-6.39 (m, 3H), 5.00-4.90 (m, 1H), 4.65 (s, |
| | 2H), 4.26 (s, 1H), 3.71-3.57 (m, 14H), 3.25-3.06 (m, 2H), 2.92-2.65 (m, 5H), 2.28-1.99 |
| | (m, 8H). |
| TDH-167 | ¹H NMR (500 MHz, CDCl₃) δ 10.08 (s, 1H), 9.03 (s, 1H), 8.58-8.45 (m, 1H), 8.01-7.91 (m, |
| | 2H), 7.84-7.77 (m, 1H), 7.68-7.61 (m, 2H), 7.59-7.54 (m, 2H), 7.44-7.40 (m, 3H), 7.27- |
| | 7.21 (m, 1H), 7.14-7.06 (m, 1H), 7.06-6.98 (m, 2H), 6.60 (s, 2H), 6.46-6.39 (m, 1H), |
| | 5.01-4.89 (m, 1H), 4.65 (s, 2H), 4.31-4.17 (m, 1H), 3.74-3.55 (m, 12H), 3.23-3.07 (m, |
| | 2H), 2.93-2.60 (m, 6H), 2.17-2.01 (m, 5H). |
| TDH-168 | ¹H NMR (500 MHz, CDCl₃) δ 10.56 (s, 1H), 9.82 (s, 0.5H), 9.04 (s, 0.5H), 8.64 (s, 0.5H), |
| | 8.54 (s, 0.5H), 8.04-7.98 (m, 1H), 7.98-7.92 (m, 1H), 7.71-7.61 (m, 3H), 7.60-7.53 (m, |
| | 1H), 7.46-7.31 (m, 3H), 7.28-7.13 (m, 2H), 7.13-7.04 (m, 1H), 7.04-6.97 (m, 1H), 6.70 |
| | (s, 1H), 6.51-6.36 (m, 1H), 4.97-4.86 (m, 1H), 4.76-4.60 (m, 2H), 4.27 (s, 1H), 3.73-3.49 |
| | (m, 6H), 3.30-3.16 (m, 2H), 2.93-2.61 (m, 5H), 2.25-2.15 (m, 4H), 2.15-1.96 (m, 3H). |
| TDH-169 | 1H NMR (500 MHz, Chloroform-d) δ 8.99 (s, 1H), 8.55 (s, 1H), 8.19-8.14 (m, 1H), 8.00 (s, |
| | 1H), 7.95 (s, 1H), 7.67-7.64 (m, 4H), 7.61-7.56 (m, 2H), 7.47-7.40 (m, 3H), 7.09-7.03 (m, |
| | 2H), 6.44 (s, 1H), 6.39 (s, 2H), 5.84-5.77 (m, 1H), 4.93 (d, J = 3.1 Hz, 2H), 4.62-4.59(m, |
| | 1H), 4.22 (s, 1H), 3.85-3.80(m, 1H), 3.17-3.09 (m, 1H), 3.07-2.95 (m, 4H), 2.91-2.82 (m, |
| | 1H), 2.74-2.65 (m, 1H), 2.42-2.39 (m, 1H), 2.29 (s, 2H), 2.19 (s, 4H), 2.12 (s, 1H), 2.09- |
| | 2.02 (m, 4H), 1.99-1.92 (m, 1H), 1.86-1.79 (m, 2H). |
| TDH-170 | 1H NMR (500 MHz, Chloroform-d) δ 9.02 (s, 1H), 8.55 (d, J = 1.4 Hz, 1H), 8.01-7.99 (m, |
| | 2H), 7.95 (s, 1H), 7.65 (d, J = 7.5 Hz, 2H), 7.60 (dd, J = 8.4, 4.8 Hz, 2H), 7.47-7.39 (m, |
| | 3H), 7.10-7.03 (m, 3H), 6.99-6.94 (m, 1H), 6.44 (s, 3H), 5.84-5.75 (m, 1H), 4.69-4.61 (m, |
| | 1H), 4.40-4.35 (m, 1H), 4.33-4.31 (m, 2H), 4.25-4.08 (m, 1H), 3.92-3.76 (m, 1H), 3.65- |
| | 3.55 (m, 1H), 3.15-3.01 (m, 4H), 2.98-2.90 (m, 2H), 2.89-2.79 (m, 1H), 2.74-2.64 (m, 1H), |
| | 2.45 (m, 1H), 2.32-2.28 (m, 2H), 2.25-2.17 (d, J = 10.8 Hz, 4H), 2.15-2.13 (m, 1H), 2.09- |
| | 2.04 (m, 2H), 1.98-1.92 (m, 1H), 1.86-1.81 (m, 2H), 1.17-1.11 (m, 2H). |
| TDH-171 | 1H NMR (400 MHz, Chloroform-d) δ 9.00 (s, 1H), 8.65-8.58 (m, 1H), 8.55 (s, 1H), 8.00 (s, |
| | 1H), 7.94 (s, 1H), 7.83 (d, J = 8.3 Hz, 1H), 7.69-7.62 (m, 2H), 7.63-7.54 (m, 2H), 7.48-7.37 |
| | (m, 4H), 7.36-7.30 (m, 1H), 7.05 (t, J = 8.5 Hz, 2H), 6.56-6.30 (m, 3H), 5.02-4.95 (m, 1H), |
| | 4.93-4.80 (m, 2H), 4.65-4.56 (m, 1H), 4.22 (s, 1H), 3.91-3.84 (m, 1H), 3.18-3.10 (m, 1H), |
| | 3.02 (s, 2H), 2.95-2.67 (m, 4H), 2.35-2.25 (m, 2H), 2.24-2.15 (m, 4H), 2.10-2.02 (m, 2H), |
| | 1.97-1.94 (m, 1H), 1.86-1.78 (m, 2H), 1.23-1.12 (m, 2H). |
| TDH-172 | 1H NMR (400 MHz, Chloroform-d) δ 9.02 (s, 1H), 8.68 (s, 1H), 8.55 (s, 1H), 8.05-7.92 (m, |
| | 3H), 7.71-7.63 (m, 2H), 7.63-7.55 (m, 2H), 7.62-7.57 (m, 1H), 7.51-7.36 (m, 4H), 7.05 (t, J = |
| | 8.7 Hz, 2H), 6.54-6.32 (s, 3H), 5.85-5.73 (m, 1H), 4.28-4.16 (m, 1H), 4.14-4.02 (m, 2H), |
| | 3.15-2.99 (m, 5H), 2.99-2.78 (m, 3H), 2.47-2.35 (m, 1H), 2.34-2.25 (m, 2H), 2.25-2.14 (m, |
| | 4H), 2.12-2.05 (m, 2H), 2.00-1.90 (m, 2H), 1.36-1.30 (m, 2H). |
| TDH-173 | ¹H NMR (500 MHz, CDCl₃) δ 9.32 (s, 1H), 9.05 (s, 1H), 8.55 (s, 1H), 8.05-7.95 (m, 3H), |
| | 7.73-7.63 (m, 2H), 7.63-7.54 (m, 2H), 7.48-7.39 (m, 4H), 7.26-7.17 (m, 2H), 7.11-6.98 |
| | (m, 2H), 6.59-6.41 (m, 3H), 5.77 (s, 1H), 5.53 (s, 1H), 4.27 (s, 1H), 3.96-3.89 (m, 2H), |
| | 3.38-3.29 (m, 2H), 3.22-3.07 (m, 2H), 3.01-2.90 (m, 2H), 2.90-2.77 (m, 1H), 2.49-2.32 |
| | (m, 3H), 2.32-1.99 (m, 10H), 1.58-1.47 (m, 6H), 1.32-1.29 (m, 6H). |
| TDH-174 | ¹H NMR (500 MHz, CDCl₃) δ 10.05 (s, 1H), 9.09 (s, 1H), 8.49 (s, 1H), 7.98-7.95 (m, 2H), |
| | 7.88-7.83 (m, 1H), 7.66-7.63 (m, 2H), 7.60-7.55 (m, 2H), 7.46-7.37 (m, 4H), 7.14-7.10 |
| | (m, 2H), 7.04-6.97 (m, 2H), 6.55 (s, 2H), 6.47-6.42 (m, 1H), 5.92 (s, 1H), 5.74-5.61 (m, |
| | 1H), 4.26 (s, 1H), 3.95-3.90 (m, 2H), 3.69-3.58 (m, 12H), 3.55-3.52 (m, 2H), 3.22-3.14 |
| | (m, 2H), 2.94-2.85 (m, 3H), 2.82-2.66 (m, 4H). |
| TDH-175 | ¹H NMR (500 MHz, CDCl₃) δ 9.16-9.00 (m, 1H), 8.55-8.39 (m, 1H), 8.02-7.90 (m, 2H), |
| | 7.76-7.60 (m, 5H), 7.60-7.47 (m, 3H), 7.47-7.35 (m, 3H), 7.20-7.10 (m, 1H), 7.10-6.94 |
| | (m, 2H), 6.56-6.28 (m, 3H), 4.98-4.88 (m, 1H), 4.62 (s, 2H), 4.20 (s, 1H), 3.80-3.58 (m, |
| | 12H), 3.58-3.47 (m, 1H), 3.28-3.16 (m, 2H), 2.92-2.60 (m, 7H), 2.28-2.16 (m, 5H). |
| TDH-176 | ¹H NMR (500 MHz, CDCl₃) δ 8.99 (s, 1H), 8.58-8.51 (m, 1H), 8.25-8.15 (m, 1H), 8.03- |
| | 7.89 (m, 3H), 7.79-7.73 (m, 1H), 7.70-7.64 (m, 2H), 7.64-7.54 (m, 3H), 7.47-7.41 (m, |
| | 3H), 7.07-7.00 (m, 2H), 6.62-6.48 (m, 2H), 6.45 (s, 1H), 5.86-5.78 (m, 1H), 4.90 (s, 1H), |
| | 4.70 (s, 2H), 4.33-4.22 (m, 1H), 3.44-3.34 (m, 2H), 3.25-3.16 (m, 2H), 3.03-2.92 (m, |
| | 2H), 2.91-2.80 (m, 1H), 2.61-2.35 (m, 5H), 2.29-2.05 (m, 10H), 1.85-1.76 (m, 3H), 1.61- |
| | 1.49 (m, 6H), 1.34-1.33 (m, 6H). |
| TDH-177 | ¹H NMR (500 MHz, CDCl₃) δ 9.09-8.83 (m, 2H), 8.54 (s, 1H), 8.05-7.93 (m, 3H), 7.69- |
| | 7.62 (m, 2H), 7.61-7.51 (m, 3H), 7.51-7.36 (m, 4H), 7.11-6.98 (m, 2H), 6.55-6.35 (m, |
| | 3H), 5.83-5.74 (m, 1H), 5.59 (s, 1H), 4.26 (s, 1H), 4.13-4.03 (m, 2H), 3.32-3.22 (m, 2H), |
| | 3.22-3.03 (m, 4H), 3.02-2.91 (m, 2H), 2.91-2.75 (m, 1H), 2.52-2.31 (m, 5H), 2.30-1.96 |
| | (m, 10H), 1.93-1.84 (m, 3H), 1.61-1.46 (m, 5H), 1.31-1.22 (m, 6H). |
| TDH-178 | ¹H NMR (500 MHz, CDCl₃) δ 9.08-8.84 (m, 2H), 8.54 (s, 1H), 8.02-7.92 (m, 3H), 7.71- |
| | 7.64 (m, 2H), 7.62-7.55 (m, 2H), 7.48-7.37 (m, 3H), 7.12-7.00 (m, 3H), 7.00-6.89 (m, |
| | 1H), 6.57-6.32 (m, 3H), 5.85-5.69 (m, 2H), 4.32-4.19 (m, 5H), 3.54-3.40 (m, 2H), 3.36- |
| | 3.28 (m, 2H), 3.21-3.03 (m, 2H), 3.01-2.87 (m, 2H), 2.87-2.73 (m, 1H), 2.53-2.32 (m, |
| | 4H), 2.30-1.96 (m, 8H), 1.61-1.46 (m, 6H), 1.31-1.26 (m, 6H). |
| TDH-179 | ¹H NMR (500 MHz, CDCl₃) δ 9.37 (s, 1H), 8.96 (s, 1H), 8.59-8.51 (m, 1H), 8.02-7.89 (m, |
| | 3H), 7.70-7.61 (m, 2H), 7.58-7.50 (m, 2H), 7.47-7.35 (m, 3H), 7.25-7.18 (m, 1H), 7.15- |
| | 7.07 (m, 1H), 7.07-6.93 (m, 2H), 6.51 (s, 2H), 6.42 (s, 1H), 5.79-5.71 (m, 1H), 4.88 (s, |
| | 1H), 4.26 (s, 1H), 3.34-3.24 (m, 2H), 3.18-3.07 (m, 2H), 2.99-2.91 (m, 2H), 2.86-2.78 |
| | (m, 1H), 2.57-2.31 (m, 5H), 2.25-1.98 (m, 10H), 1.58-1.49 (m, 4H), 1.45-1.34 (m, 6H). |
| TDH-180 | 1H NMR (300 MHz, Chloroform-d) δ 9.10 (s, 1H), 8.89 (s, 1H), 8.01 (s, 1H), 7.70-7.62 (m, |
| | 2H), 7.61-7.54 (m, 2H), 7.51-7.40 (m, 3H), 7.02-6.96 (m, 2H), 6.65-6.40 (m, 3H), 4.75- |
| | 4.62 (m, 1H), 4.32 (m, 2H), 3.05-2.89 (m, 2H), 2.31-2.09 (m, 2H), 2.08-1.92 (m, 3H), 1.50 |
| | (s, 9H). LC/MS (ESI) m/z 617[M + H]+ |
| TDH-181 | 1H NMR (300 MHz, Methanol-d4) δ 8.90 (s, 1H), 8.54 (s, 1H), 7.65-7.59 (m, 2H), 7.57- |
| | 7.51 (m, 2H), 7.43-7.32 (m, 3H), 7.11-7.03 (m, 2H), 5.16 (s, 1H), 4.61 (s, 1H), 3.51-3.42 |
| | (m, 2H), 3.16-3.06 (m, 2H), 2.46-2.35 (m, 2H), 2.34-2.28 (m, 2H). LC/MS (ESI) m/z 517 [M + H]+ |
| TDH-182 | 1H NMR (300 MHz, Chloroform-d) δ 9.09 (s, 1H), 8.94 (s, 1H), 8.05 (s, 1H), 7.76-7.7.65 |
| | (m, 2H), 7.62-7.54 (m, 2H), 7.54-7.36 (m, 3H), 7.05 (t, J = 8.5 Hz, 2H), 6.66-6.47 (m, 3H), |
| | 4.69-4.50 (m, 1H), 3.06 (d, J = 10.7 Hz, 2H), 2.40 (s, 3H), 2.32-3.21 (m, 4H). LC/MS (ESI) |
| | m/z 531 [M + H]+ |
| TDH-183 | 1H NMR (500 MHz, CDCl3) δ 9.01 (s, 1H), 8.82 (d, J = 2.4 Hz, 1H), 8.66 (s, 1H), 8.05 (dd, |
| | J = 8.8, 2.5 Hz, 1H), 7.64-7.60 (m, 2H), 7.58-7.52 (m, 2H), 7.44-7.35 (m, 3H), 7.01-6.94 |
| | (m, 2H), 6.75 (d, J = 8.9 Hz, 1H), 6.42 (s, 1H), 6.34 (s, 2H), 3.75-3.65 (m, 4H), 2.67-2.59 |
| | (m, 4H), 2.53 (q, J = 7.2 Hz, 2H), 1.17 (t, J = 7.2 Hz, 3H). LC/MS (ESI) m/z 556 [M + H]+ |
| TDH-184 | 1H NMR (400 MHz, CDCl3) δ 8.98 (s, 1H), 8.86 (s, 1H), 8.70 (s, 1H), 8.20-8.07 (m, 1H), |
| | 7.75-7.64 (m, 2H), 7.62-7.51 (m, 2H), 7.51-7.36 (m, 3H), 7.07-6.97 (m, 2H), 6.78 (d, J = |
| | 8.9 Hz, 1H), 6.54-6.26 (m, 3H), 3.87-3.74 (m, 4H), 3.71-3.58 (m, 4H), 2.20 (s, 3H). |
| | LC/MS (ESI) m/z 570 [M + H]+ |
| TDH-185 | 1H NMR (500 MHz, CDCl3) δ 8.82 (s, 1H), 8.51 (s, 1H), 8.08 (s, 1H), 8.00-7.92 (m, 1H), |
| | 7.70-7.60 (m, 2H), 7.57-7.49 (m, 2H), 7.49-7.37 (m, 4H), 7.06-6.97 (m, 2H), 6.50-6.23 |
| | (m, 3H), 5.19-5.08 (m, 1H), 4.65-4.58 (m, 2H), 4.55-4.49 (m, 1H), 4.47-4.40 (m, 1H), |
| | 1.98-1.95 (m, 3H). LC/MS (ESI) m/z 530 [M + H]+ |
| TDH-186 | ¹H NMR (500 MHz, CDCl₃) δ 9.13-9.05 (m, 1H), 8.41 (s, 1H), 7.98-7.88 (m, 3H), 7.67 (t, J = |
| | 7.9 Hz, 1H), 7.59-7.52 (m, 2H), 7.49 (d, J = 7.3 Hz, 1H), 7.47-7.35 (m, 5H), 7.32 (t, J = |
| | 8.1 Hz, 1H), 7.03-6.94 (m, 2H), 6.53 (s, 2H), 5.79-5.68 (m, 1H), 5.08-4.89 (m, 3H), 4.57- |
| | 4.46 (m, 1H), 4.22-4.15 (m, 1H), 4.10-3.98 (m, 1H), 3.14-3.04 (m, 1H), 3.03-2.92 (m, |
| | 2H), 2.92-2.69 (m, 3H), 2.69-2.59 (m, 1H), 2.27-2.19 (m, 2H), 2.18-2.08 (m, 7H), 1.93- |
| | 1.86 (m, 1H), 1.83-1.74 (m, 2H), 1.15-1.03 (m, 2H). |
| TDH-187 | 1H NMR (400 MHz, CDCl3) δ 9.08 (d, J = 7.1 Hz, 1H), 8.65 (s, 1H), 8.41 (s, 1H), 7.97- |
| | 7.83 (m, 3H), 7.79 (d, J = 8.3 Hz, 1H), 7.63-7.52 (m, 2H), 7.51-7.33 (m, 6H), 7.32-7.28 |
| | (m, 1H), 7.04-6.95 (m, 2H), 6.57 (s, 2H), 5.73 (d, J = 7.1 Hz, 1H), 5.04-4.92 (m, 1H), |
| | 4.92-4.77 (m, 2H), 4.63-4.51 (m, 1H), 4.25-4.16 (m, 1H), 3.93-3.81 (m, 1H), 3.16-3.06 |
| | (m, 1H), 3.06-2.95 (m, 2H), 2.94-2.72 (m, 3H), 2.72-2.62 (m, 1H), 2.33-2.22 (m, 2H), |
| | 2.22-2.06 (m, 7H), 1.99-1.90 (m, 1H), 1.87-1.76 (m, 2H), 1.20-1.08 (m, 2H). |
| TDH-188 | ¹H NMR (500 MHz, DMSO) δ 11.12 (s, 1H), 10.66 (s, 1H), 8.74-8.64 (m, 1H), 8.34-8.27 |
| | (m, 1H), 7.99-7.94 (m, 1H), 7.82-7.76 (m, 1H), 7.76-7.71 (m, 2H), 7.66-7.58 (m, 2H), |
| | 7.53-7.41 (m, 4H), 7.40-7.36 (m, 1H), 7.32 (s, 2H), 7.20-7.12 (m, 2H), 6.25 (s, 1H), 5.27 |
| | (s, 2H), 5.15-5.09 (m, 1H), 4.60-4.48 (m, 2H), 4.48-4.35 (m, 2H), 4.00-3.90 (m, 2H), |
| | 3.88 (s, 2H), 3.34-3.24 (m, 2H), 2.96-2.82 (m, 3H), 2.67-2.54 (m, 2H), 2.18-1.99 (m, |
| | 6H), 1.99-1.73 (m, 3H). |
| TDH-189 | ¹H NMR (500 MHz, DMSO) δ 11.13 (s, 1H), 10.65 (s, 1H), 8.73-8.65 (m, 1H), 8.33-8.28 |
| | (m, 1H), 7.97-7.95 (m, 1H), 7.88-7.81 (m, 1H), 7.77-7.70 (m, 2H), 7.65-7.59 (m, 2H), |
| | 7.54-7.46 (m, 3H), 7.46-7.41 (m, 1H), 7.40-7.36 (m, 1H), 7.33-7.28 (m, 2H), 7.20-7.12 |
| | (m, 2H), 6.25 (s, 1H), 5.20 (s, 2H), 5.16-5.12 (m, 1H), 4.62-4.46 (m, 2H), 4.48-4.39 (m, |
| | 1H), 4.00-3.91 (m, 1H), 3.88 (s, 2H), 2.96-2.83 (m, 6), 2.67-2.54 (m, 3H), 2.14-1.99 (m, |
| | 6H), 1.96-i.69 (m, 3H). |
| TDH-190 | ¹H NMR (500 MHz, DMSO) δ 11.12 (s, 1H), 10.64 (s, 1H), 8.72-8.65 (m, 1H), 8.34-8.29 |
| | (m, 1H), 7.98-7.94 (m, 1H), 7.76-7.71 (m, 2H), 7.66-7.58 (m, 3H), 7.53-7.47 (m, 2H), |
| | 7.46-7.39 (m, 1H), 7.32-7.28 (m, 2H), 7.18-7.11 (m, 4H), 7.10-7.07 (m, 1H), 6.24 (s, |
| | 1H), 5.12-5.07 (m, 1H), 4.60-4.47 (m, 3H), 4.34-4.20 (m, 3H), 4.10-3.98 (m, 2H), 3.87 |
| | (s, 2H), 3.31-3.22 (m, 2H), 2.97-2.84 (m, 3H), 2.68-2.54 (m, 3H), 2.19-2.09 (m, 3H), |
| | 2.09-1.97 (m, 3H), 1.92-1.80 (m, 2H). |
| TDH-191 | ¹H NMR (500 MHz, DMSO) δ 11.08 (s, 1H), 10.64 (s, 1H), 8.74-8.65 (m, 1H), 8.34-8.29 |
| | (m, 1H), 8.00-7.92 (m, 1H), 7.79-7.68 (m, 2H), 7.67-7.58 (m, 3H), 7.55-7.47 (m, 2H), |
| | 7.47-7.39 (m, 1H), 7.34-7.28 (m, 2H), 7.20-7.10 (m, 4H), 7.09-7.02 (m, 1H), 6.24 (s, |
| | 1H), 5.09-5.02 (m, 1H), 4.61-4.42 (m, 3H), 4.29-4.15 (m, 3H), 4.16-4.05 (m, 2H), 3.88 |
| | (s, 2H), 3.31-3.17 (m, 2H), 2.95-2.78 (m, 3H), 2.65-2.52 (m, 3H), 2.21-2.07 (m, 3H), |
| | 2.07-1.94 (m, 3H), 1.90-1.73 (m, 2H). |
| TDH-192 | ¹H NMR (500 MHz, DMSO) δ 11.21-11.03 (m, 1H), 10.65 (s, 1H), 8.74-8.63 (m, 1H), |
| | 8.32-8.25 (m, 0.5H), 8.15-8.06 (m, 0.5H), 7.96-7.90 (m, 0.5H), 7.87-7.79 (m, 0.5H), |
| | 7.78-7.69 (m, 2H), 7.65-7.59 (m, 3H), 7.52-7.46 (m, 2H), 7.46-7.38 (m, 1H), 7.35-7.27 |
| | (m, 2H), 7.18-7.14 (m, 2H), 7.08-6.99 (m, 1H), 6.82-6.74 (m, 1H), 6.24 (s, 1H), 5.11- |
| | 4.97 (m, 1H), 4.97-4.84 (m, 1H), 4.61-4.32 (m, 3H), 4.18-4.08 (m, 1H), 3.73-3.69 (m, |
| | 1H), 3.69-3.59 (m, 1H), 3.59-3.46 (m, 3H), 3.44-3.36 (m, 1H), 3.31-3.24 (m, 1H), 3.22- |
| | 3.11 (m, 2H), 2.97-2.54 (m, 2H), 2.51-2.40 (m, 9H), 2.16-1.99 (m, 2H), 1.99-1.88 (m, 2H). |
| TDH-193 | ¹H NMR (500 MHz, DMSO) δ 11.13-11.02 (m, 1H), 10.65 (s, 1H), 8.74-8.67 (m, 1H), 8.27 |
| | (s, 0.5H), 8.18 (s, 0.5H), 7.97-7.87 (m, 1H), 7.79-7.70 (m, 2H), 7.65-7.59 (m, 2H), 7.52- |
| | 7.46 (m, 2H), 7.46-7.37 (m, 1H), 7.36-7.28 (m, 2H), 7.28-7.20 (m, 1H), 7.20-7.12 (m, |
| | 2H), 7.05-6.98 (m, 1H), 6.94-6.83 (m, 1H), 6.24 (s, 1H), 5.09-4.97 (m, 1H), 4.97-4.87 |
| | (m, 1H), 4.60-4.40 (m, 2H), 4.14-3.98 (m, 1H), 3.73-3.68 (m, 1H), 3.68-3.57 (m, 1H), |
| | 3.57-3.49 (m, 1H), 3.49-3.36 (m, 1H), 3.32-3.24 (m, 2H), 3.21-3.11 (m, 2H), 2.93-2.68 |
| | (m, 2H), 2.66-2.44 (m, 10H), 2.12-1.85 (m, 4H). |
| TDH-194 | ¹H NMR (500 MHz, DMSO) δ 11.10 (s, 1H), 10.65 (s, 1H), 8.74-8.69 (m, 1H), 8.36-8.29 |
| | (m, 1H), 7.97-7.91 (m, 1H), 7.77-7.66 (m, 3H), 7.66-7.58 (m, 2H), 7.54-7.47 (m, 2H), |
| | 7.47-7.41 (m, 1H), 7.38-7.29 (m, 4H), 7.21-7.13 (m, 2H), 6.24 (s, 1H), 5.16-5.08 (m, |
| | 1H), 4.60-4.48 (m, 2H), 4.24-4.09 (m, 1H), 3.79-3.69 (m, 3H), 3.31-3.15 (m, 2H), 3.07- |
| | 2.82 (m, 5H), 2.82-2.72 (m, 1H), 2.66-2.51 (m, 8H), 2.21-1.99 (m, 4H), 1.98-1.69 (m, 7H). |
| TDH-195 | ¹H NMR (500 MHz, DMSO) δ 11.09 (s, 1H), 10.65 (s, 1H), 8.74-8.68 (m, 1H), 8.35-8.28 |
| | (m, 1H), 7.98-7.93 (m, 1H), 7.79-7.71 (m, 2H), 7.72-7.57 (m, 3H), 7.52-7.47 (m, 2H), |
| | 7.47-7.40 (m, 1H), 7.36-7.29 (m, 3H), 7.29-7.22 (m, 1H), 7.21-7.10 (m, 2H), 6.24 (s, |
| | 1H), 5.13-5.04 (m, 1H), 4.58-4.46 (m, 2H), 4.25-4.14 (m, 1H), 4.14-4.04 (m, 2H), 3.71 |
| | (s, 1H), 3.32-3.21 (m, 1H), 3.17-3.01 (m, 3H), 2.94-2.83 (m, 2H), 2.82-2.73 (m, 2H), |
| | 2.64-2.51 (m, 7H), 2.20-2.13 (m, 1H), 2.13-1.97 (m, 3H), 1.97-1.84 (m, 2H), 1.84-1.71 |
| | (m, 4H), 1.71-1.59 (m, 2H). |
| TDH-196 | ¹H NMR (500 MHz, DMSO) δ 11.09 (s, 1H), 10.63 (s, 1H), 8.74-8.67 (m, 1H), 8.32-8.25 |
| | (m, 1H), 7.95-7.89 (m, 1H), 7.76-7.65 (m, 3H), 7.65-7.57 (m, 2H), 7.53-7.46 (m, 2H), |
| | 7.46-7.40 (m, 1H), 7.37-7.23 (m, 4H), 7.21-7.11 (m, 2H), 6.23 (s, 1H), 5.14-5.06 (m, |
| | 1H), 4.44-4.37 (m, 1H), 4.23-4.14 (m, 1H), 4.04-3.96 (m, 1H), 3.74-3.69 (m, 2H), 3.09- |
| | 2.93 (m, 5H), 2.90-2.82 (m, 2H), 2.62-2.54 (m, 2H), 2.41-2.32 (m, 2H), 2.07-1.93 (m, |
| | 7H), 1.81-1.64 (m, 6H), 1.62-1.50 (m, 2H), 1.46-1.39 (m, 2H), 1.12-1.04 (m, 1H), 1.02- |
| | 0.92 (m, 1H). |
| TDH-197 | ¹H NMR (500 MHz, DMSO) δ 11.09 (s, 1H), 10.65 (s, 1H), 8.73-8.69 (m, 1H), 8.33-8.25 |
| | (m, 1H), 7.96-7.87 (m, 1H), 7.77-7.70 (m, 2H), 7.70-7.59 (m, 3H), 7.52-7.47 (m, 2H), |
| | 7.47-7.40 (m, 1H), 7.36-7.27 (m, 3H), 7.27-7.21 (m, 1H), 7.19-7.11 (m, 2H), 6.33-6.16 |
| | (m, 1H), 5.13-5.03 (m, 1H), 4.45-4.36 (m, 1H), 4.28-4.15 (m, 1H), 4.13-3.98 (m, 3H), |
| | 3.09-2.84 (m, 7H), 2.64-2.54 (m, 2H), 2.43-2.31 (m, 2H), 2.07-1.94 (m, 7H), 1.82-1.75 |
| | (m, 1H), 1.72-1.65 (m, 3H), 1.64-1.53 (m, 3H), 1.47-1.40 (m, 2H), 1.14-1.05 (m, 1H), |
| | 1.00-0.91 (m, 1H). |
| TDH-198 | ¹H NMR (500 MHz, DMSO) δ 11.07 (s, 1H), 10.64 (s, 1H), 8.74-8.67 (m, 1H), 8.33-8.25 |
| | (m, 1H), 7.96-7.88 (m, 1H), 7.77-7.71 (m, 1H), 7.68-7.55 (m, 3H), 7.53-7.41 (m, 3H), |
| | 7.38-7.25 (m, 3H), 7.21-7.10 (m, 3H), 7.10-6.99 (m, 2H), 6.23 (s, 1H), 5.09-5.00 (m, |
| | 1H), 4.41-4.33 (m, 1H), 4.25-4.05 (m, 4H), 3.98-3.91 (m, 1H), 3.07-2.95 (m, 3H), 2.92- |
| | 2.83 (m, 1H), 2.69-2.52 (m, 4H), 2.46-2.33 (m, 2H), 2.07-1.97 (m, 6H), 1.78-1.70 (m, |
| | 2H), 1.46-1.40 (m, 2H), 1.20-1.11 (m, 1H), 1.07-0.95 (m, 1H). |
| TDH-199 | ¹H NMR (500 MHz, DMSO) δ 11.11 (s, 1H), 10.63 (s, 1H), 8.72-8.67 (m, 1H), 8.34-8.26 |
| | (m, 1H), 7.94-7.89 (m, 1H), 7.74-7.71 (m, 1H), 7.66-7.58 (m, 3H), 7.51-7.41 (m, 3H), |
| | 7.35-7.23 (m, 3H), 7.20-7.09 (m, 4H), 7.09-7.05 (m, 1H), 6.23 (s, 1H), 5.11-5.04 (m, |
| | 1H), 4.43-4.34 (m, 1H), 4.24-4.08 (m, 4H), 3.91-3.87 (m, 1H), 3.05-2.84 (m, 5H), 2.68- |
| | 2.55 (m, 3H), 2.41-2.32 (m, 2H), 2.06-1.97 (m, 6H), 1.78-1.72 (m, 2H), 1.46-1.40 (m, |
| | 2H), 1.20-1.15 (m, 1H), 1.07-0.99 (m, 1H). |
| TDH-200 | ¹H NMR (500 MHz, DMSO) δ 11.12 (s, 1H), 10.64 (s, 1H), 8.73-8.68 (m, 1H), 8.31-8.25 |
| | (m, 1H), 7.95-7.89 (m, 1H), 7.80-7.74 (m, 1H), 7.75-7.69 (m, 2H), 7.66-7.59 (m, 2H), |
| | 7.52-7.46 (m, 2H), 7.46-7.40 (m, 2H), 7.33-7.27 (m, 3H), 7.20-7.12 (m, 2H), 6.24 (s, |
| | 1H), 5.20-5.09 (m, 3H), 4.32-4.24 (m, 1H), 4.24-4.13 (m, 1H), 3.84-3.73 (m, 1H), 3.13- |
| | 2.82 (m, 5H), 2.66-2.53 (m, 3H), 2.43-2.31 (m, 2H), 2.06-1.96 (m, 6H), 1.75-1.68 (m, |
| | 2H), 1.46-1.37 (m, 2H), 1.23-1.15 (m, 2H), 1.05-0.94 (m, 1H). |
| TDH-201 | ¹H NMR (500 MHz, DMSO) δ 11.13 (s, 1H), 10.64 (s, 1H), 8.74-8.68 (m, 1H), 8.35-8.27 |
| | (m, 1H), 7.98-7.90 (m, 1H), 7.89-7.81 (m, 1H), 7.77-7.71 (m, 2H), 7.70-7.61 (m, 2H), |
| | 7.52-7.48 (m, 2H), 7.46-7.42 (m, 2H), 7.39-7.25 (m, 3H), 7.20-7.13 (m, 2H), 6.24 (s, |
| | 1H), 5.18-5.07 (m, 3H), 4.33-4.25 (m, 1H), 4.25-4.15 (m, 1H), 3.84-3.76 (m, 1H), 3.11- |
| | 2.85 (m, 5H), 2.67-2.55 (m, 3H), 2.43-2.34 (m, 2H), 2.07-1.94 (m, 6H), 1.81-1.70 (m, |
| | 2H), 1.48-1.38 (m, 2H), 1.22-1.12 (m, 2H), 1.03-0.96 (m, 1H). |
| TDH-202 | ¹H NMR (500 MHz, DMSO) δ 11.09 (s, 1H), 10.63 (s, 1H), 8.74-8.67 (m, 1H), 8.34-8.26 |
| | (m, 1H), 7.96-7.89 (m, 1H), 7.74-7.71 (m, 1H), 7.71-7.66 (m, 1H), 7.66-7.59 (m, 2H), |
| | 7.53-7.47 (m, 2H), 7.47-7.40 (m, 1H), 7.38-7.26 (m, 5H), 7.20-7.09 (m, 2H), 6.23 (s, |
| | 1H), 5.13-5.05 (m, 1H), 4.26-4.15 (m, 1H), 3.73-3.64 (m, 3H), 3.07-2.94 (m, 2H), 2.89- |
| | 2.82 (m, 3H), 2.66-2.53 (m, 3H), 2.46-2.35 (m, 2H), 2.06-1.97 (m, 5H), 1.85-1.77 (m, |
| | 2H), 1.53-1.43 (m, 3H), 1.43-1.32 (m, 2H). |
| TDH-203 | ¹H NMR (500 MHz, DMSO) δ 11.09 (s, 1H), 10.65 (s, 1H), 8.73-8.68 (m, 1H), 8.32-8.27 |
| | (m, 1H), 7.95-7.90 (m, 1H), 7.76-7.70 (m, 2H), 7.67-7.60 (m, 3H), 7.52-7.47 (m, 2H), |
| | 7.46-7.41 (m, 1H), 7.34-7.29 (m, 3H), 7.26-7.22 (m, 1H), 7.20-7.12 (m, 2H), 6.24 (s, |
| | 1H), 5.11-5.03 (m, 1H) 4.27-4.16, (m, 1H), 4.09-4.01 (m, 3H), 3.01-2.84 (m, 5H), 2.65- |
| | 2.52 (m, 3H), 2.46-2.36 (m, 2H), 2.05-1.98 (m, 5H), 1.81-1.76 (m, 2H), 1.26-1.16 (m, |
| | 5H). |
| TDH-204 | ¹H NMR (500 MHz, DMSO) δ 11.09 (s, 1H), 10.63 (s, 1H), 8.69 (s, 1H), 8.30 (s, 1H), 7.91 |
| | (s, 1H), 7.76-7.71 (m, 2H), 7.71-7.65 (m, 1H), 7.64-7.59 (m, 2H), 7.52-7.47 (m, 2H), |
| | 7.46-7.41 (m, 1H), 7.36-7.29 (m, 4H), 7.20-7.12 (m, 2H), 6.23 (s, 1H), 5.14-5.04 (m, |
| | 1H), 4.45-4.38 (m, 1H), 4.23-4.14 (m, 1H), 3.96-3.89 (m, 1H), 3.70-3.66 (m, 2H), 2.99- |
| | 2.82 (m, 7H), 2.64-2.53 (m, 3H), 2.34-2.28 (m, 2H), 2.21-2.14 (m, 2H), 2.07-1.97 (m, |
| | 6H), 1.82-1.69 (m, 6H), 1.44-1.36 (m, 2H), 1.08-0.97 (m, 1H), 0.97-0.86 (m, 1H). |
| TDH-205 | ¹H NMR (500 MHz, DMSO) δ 11.08 (s, 1H), 10.63 (s, 1H), 8.69 (s, 1H), 8.29 (s, 1H), 7.91 |
| | (s, 1H), 7.75-7.70 (m, 2H), 7.67-7.57 (m, 3H), 7.52-7.46 (m, 2H), 7.46-7.39 (m, 1H), |
| | 7.33-7.26 (m, 3H), 7.25-7.20 (m, 1H), 7.19-7.12 (m, 2H), 6.23 (s, 1H), 5.10-5.03 (m, |
| | 1H), 4.45-4.36 (m, 1H), 4.23-4.14 (m, 1H), 4.07-4.01 (m, 2H), 3.93-3.87 (m, 1H), 3.01- |
| | 2.85 (m, 7H), 2.65-2.53 (m, 3H), 2.29-2.25 (m, 2H), 2.20-2.14 (m, 2H), 2.07-1.99 (m, |
| | 6H), 1.80-1.68 (m, 6H), 1.25-1.19 (m, 2H), 1.05-0.95 (m, 1H), 0.95-0.85 (m, 1H). |
| TDH-206 | 1H NMR (500 MHz, CDCl3) δ 9.11 (d, J = 7.1 Hz, 1H), 8.56-8.46 (m, 1H), 8.43 (s, 1H), |
| | 7.93 (d, J = 9.5 Hz, 2H), 7.80 (s, 1H), 7.58 (d, J = 7.5 Hz, 2H), 7.53 (t, J = 7.8 Hz, 1H), |
| | 7.50-7.43 (m, 4H), 7.43-7.38 (m, 1H), 7.27-7.23 (m, 1H), 7.15 (d, J = 7.2 Hz, 1H), 7.02 (t, |
| | J = 8.4 Hz, 2H), 6.82 (d, J = 8.4 Hz, 1H), 5.75 (d, J = 7.0 Hz, 1H), 5.00-4.92 (m, 1H), |
| | 4.72-4.63 (m, 1H), 4.25-4.17 (m, 1H), 4.10-4.01 (m, 2H), 3.84-3.75 (m, 1H), 3.17-3.08 |
| | (m, 1H), 3.08-2.97 (m, 2H), 2.94-2.74 (m, 3H), 2.74-2.68 (m, 1H), 2.28 (d, J = 6.9 Hz, |
| | 2H), 2.21-2.11 (m, 7H), 2.08-2.03 (m, 2H), 2.00-1.94 (m, 1H), 1.90-1.84 (m, 1H), 1.23- |
| | 1.12 (m, 2H). |
| TDH-207 | 1H NMR (500 MHz, CDCl3) δ 9.09 (d, J = 7.1 Hz, 1H), 8.56-8.46 (m, 1H), 8.42 (s, 1H), |
| | 7.92 (d, J = 7.9 Hz, 2H), 7.81 (s, 1H), 7.63 (d, J = 8.3 Hz, 1H), 7.56 (d, J = 7.5 Hz, 2H), |
| | 7.49-7.36 (m, 5H), 7.00 (t, J = 8.4 Hz, 2H), 6.95 (s, 1H), 6.83 (d, J = 8.3 Hz, 1H), 5.98- |
| | 5.90 (m, 1H), 5.73 (d, J = 7.1 Hz, 1H), 4.98-4.91 (m, 1H), 4.69-4.60 (m, 1H), 4.24-4.14 |
| | (m, 1H), 4.01-3.90 (m, 2H), 3.81-3.73 (m, 1H), 3.15-3.06 (m, 1H), 3.06-2.96 (m, 2H), |
| | 2.93-2.70 (m, 4H), 2.26 (d, J = 7.0 Hz, 2H), 2.20-2.09 (m, 7H), 2.06-2.01 (m, 2H), 2.00- |
| | 1.94 (m, 1H), 1.91-1.85 (m, 1H), 1.20-1.12 (m, 2H). |
| TDH-208 | ¹H NMR (500 MHz, CDCl₃) δ 9.08 (d, J = 7.2 Hz, 1H), 8.79 (s, 1H), 8.41 (s, 1H), 8.00 (s, |
| | 1H), 7.91 (d, J = 5.9 Hz, 2H), 7.60-7.52 (m, 3H), 7.48-7.39 (m, 4H), 7.39-7.33 (m, 2H), |
| | 7.18 (d, J = 8.4 Hz, 1H), 6.97 (t, J = 8.4 Hz, 2H), 5.75 (d, J = 7.2 Hz, 1H), 5.03-4.93 (m, |
| | 1H), 4.24-4.13 (m, 1H), 3.81-3.70 (m, 2H), 3.12-2.97 (m, 2H), 2.95-2.67 (m, 5H), 2.37- |
| | 2.25 (m, 2H), 2.21-2.07 (m, 7H), 1.97-1.89 (m, 2H), 1.74-1.69 (m, 1H), 1.53-1.42 (m, 2H). |
| TDH-209 | 1H NMR (500 MHz, CDCl3) δ 9.08 (d, J = 7.2 Hz, 1H), 8.76 (s, 1H), 8.40 (s, 1H), 8.07 (s, |
| | 1H), 7.91 (d, J = 7.7 Hz, 2H), 7.60-7.52 (m, 3H), 7.48-7.39 (m, 4H), 7.36 (t, J = 7.5 Hz, |
| | 2H), 7.17 (d, J = 8.5 Hz, 1H), 6.98 (t, J = 8.4 Hz, 2H), 5.76 (d, J = 7.2 Hz, 1H), 5.02-4.93 |
| | (m, 1H), 4.72-4.58 (m, 1H), 4.24-4.12 (m, 1H), 4.02-3.88 (m, 1H), 3.88-3.75 (m, 2H), |
| | 3.14-2.94 (m, 5H), 2.93-2.77 (m, 2H), 2.77-2.67 (m, 2H), 2.64-2.51 (m, 1H), 2.24 (d, J = |
| | 6.8 Hz, 2H), 2.20-2.03 (m, 10H), 1.90-1.82 (m, 2H), 1.82-1.75 (m, 2H), 1.19-1.04 (m, 2H). |
| TDH-210 | 1H NMR (500 MHz, CDCl3) δ 9.09 (d, J = 7.2 Hz, 1H), 8.99-8.90 (m, 1H), 8.41 (s, 1H), |
| | 8.14 (s, 1H), 7.96-7.87 (m, 2H), 7.66 (d, J = 8.5 Hz, 1H), 7.56 (d, J = 7.5 Hz, 2H), 7.49- |
| | 7.34 (m, 5H), 7.26-7.23 (m, 1H), 7.04 (d, J = 8.7 Hz, 1H), 6.97 (t, J = 8.4 Hz, 2H), 5.77 (d, |
| | J = 7.3 Hz, 1H), 5.01-4.91 (m, 1H), 4.71-4.55 (m, 1H), 4.26-4.09 (m, 1H), 4.05-3.85 (m, |
| | 3H), 3.12-2.95 (m, 5H), 2.92-2.69 (m, 4H), 2.66-2.52 (m, 1H), 2.25 (d, J = 6.9 Hz, 2H), |
| | 2.20-2.07 (m, 7H), 2.01-1.89 (m, 3H), 1.89-1.78 (m, 4H), 1.19-1.05 (m, 2H). |
| TDH-211 | 1H NMR (300 MHz, CDCl3) δ 9.01 (s, 1H), 8.68 (s, 1H), 8.52 (s, 1H), 7.98-7.87 (m, 3H), |
| | 7.68-7.62 (m, 2H), 7.56 (dd, J = 8.9, 4.8 Hz, 2H), 7.46-7.31 (m, 8H), 7.02 (t, J = 8.6 Hz, |
| | 1H), 6.42 (s, 1H), 6.32 (s, 2H), 4.77 (t, J = 7.9 Hz, 1H), 4.56 (m, 2H), 4.45-4.29 (m, 2H), |
| | 4.27-4.06 (m, 3H), 3.78 (s, 1H), 3.62-3.54 (m, 1H), 3.48 (s, 1H), 2.97 (m, 4H), 2.85 (d, J = |
| | 11.2 Hz, 2H), 2.58 (m, 1H), 2.52 (s, 3H), 2.13 (m, 7H), 1.78 (m, 7H), 0.95 (s, 9H). |
| TDH-212 | 1H NMR (500 MHz, MeOD) δ 8.90 (s, 1H), 8.60 (m, 1H), 8.25 (m, 1H), 8.01 (m, 1H), 7.73 |
| | (d, J = 7.5 Hz, 1H), 7.69-7.58 (m, 1H), 7.53-7.37 (m, 9H), 7.07 (t, J = 8.3 Hz, 1H), 6.35 |
| | (s, 1H), 5.07 (m, 3H), 4.68 (m, 2H), 4.56-4.40 (m, 2H), 4.22 (s, 1H), 3.98 (t, J = 12.5 Hz, |
| | 2H), 3.86 (d, J = 9.0 Hz, 1H), 3.16-3.00 (m, 3H), 2.76-2.53 (m, 5H), 2.49 (m, 4H), 2.26 |
| | (m, 4H), 2.08 (m, 6H), 1.95-1.73 (m, 3H), 1.13 (s, H). |
| TDH-213 | 1H NMR (400 MHz, CDCl3) δ 9.59 (s, 1H), 9.07 (d, J = 7.2 Hz, 1H), 8.41-8.31 (m, 1H), |
| | 8.13 (s, 1H), 7.99-7.90 (m, 1H), 7.88 (s, 1H), 7.56 (d, J = 7.5 Hz, 2H), 7.52-7.32 (m, 6H), |
| | 7.30 (d, J = 7.1 Hz, 1H), 7.06 (t, J = 7.8 Hz, 1H), 6.96 (t, J = 8.5 Hz, 2H), 6.63-6.54 (m, |
| | 1H), 5.79-5.69 (m, 1H), 5.00-4.88 (m, 1H), 4.29-4.14 (m, 1H), 3.76-3.60 (m, 8H), 3.60- |
| | 3.53 (m, 2H), 3.52-3.42 (m, 2H), 3.27-3.13 (m, 2H), 2.95-2.80 (m, 3H), 2.80-2.63 (m, |
| | 4H), 2.41-2.26 (m, 3H), 2.25-2.13 (m, 4H), 2.11-2.02 (m, 2H), 1.99-1.94 (m, 3H). |
| TDH-214 | 1H NMR (400 MHz, CDCl3) δ 9.27 (s, 1H), 9.02 (d, J = 7.3 Hz, 1H), 8.37-8.25 (m, 2H), |
| | 7.95 (s, 1H), 7.90 (s, 1H), 7.56 (d, J = 7.6 Hz, 2H), 7.53-7.44 (m, 3H), 7.43-7.31 (m, 3H), |
| | 7.03 (s, 1H), 7.01-6.93 (m, 2H), 6.88-6.76 (m, 2H), 6.64 (s, 2H), 5.74 (t, J = 7.9 Hz, 1H), |
| | 5.00-4.90 (m, 1H), 4.29-4.16 (m, 1H), 3.85-3.72 (m, 2H), 3.71-3.61 (m, 6H), 3.61-3.53 |
| | (m, 2H), 3.51-3.42 (m, 2H), 3.27-3.15 (m, 2H), 2.92-2.65 (m, 7H), 2.43-2.32 (m, 2H), |
| | 2.27-2.10 (m, 6H), 2.08-2.01 (m, 1H), 1.81-1.68 (m, 2H). |
| TDH-215 | 1H NMR (500 MHz, CDCl3) δ 9.08 (t, J = 6.7 Hz, 1H), 8.39 (s, 1H), 8.00-7.91 (m, 2H), |
| | 7.89 (s, 1H), 7.55 (d, J = 7.5 Hz, 2H), 7.50-7.33 (m, 6H), 7.07 (t, J = 7.9 Hz, 1H), 6.97 (t, J = |
| | 8.5 Hz, 2H), 6.93-6.87 (m, 1H), 6.55-6.47 (m, 1H), 5.73 (d, J = 7.1 Hz, 1H), 4.92-4.85 |
| | (m, 1H), 4.25-4.14 (m, 1H), 3.77-3.70 (m, 2H), 3.70-3.61 (m, 6H), 3.51-3.42 (m, 2H), |
| | 3.20-3.08 (m, 2H), 2.87-2.61 (m, 5H), 2.33-2.25 (m, 2H), 2.21-2.12 (m, 4H), 2.12-1.99 |
| | (m, 1H). |
| TDH-216 | 1H NMR (500 MHz, CDCl3) δ 9.09 (t, J = 7.1 Hz, 1H), 8.93 (s, 1H), 8.45-8.39 (m, 1H), |
| | 7.99-7.95 (m, 1H), 7.95-7.86 (m, 2H), 7.61-7.51 (m, 3H), 7.49-7.33 (m, 6H), 7.05-6.93 |
| | (m, 3H), 6.76 (d, J = 8.4 Hz, 1H), 5.72-5.65 (m, 1H), 5.53-5.42 (m, 1H), 4.94-4.87 (m, |
| | 1H), 4.27-4.16 (m, 1H), 3.78-3.71 (m, 2H), 3.70-3.59 (m, 6H), 3.42-3.35 (m, 2H), 3.22- |
| | 3.11 (m, 2H), 2.89-2.65 (m, 5H), 2.26-2.13 (m, 5H), 2.10-1.99 (m, 2H). |
| TDH-217 | 1H NMR (500 MHz, CDCl3) δ 9.15 (d, J = 7.7 Hz, 1H), 8.37 (s, 1H), 8.01 (s, 1H), 7.90 (s, |
| | 1H), 7.57 (d, J = 7.5 Hz, 2H), 7.54-7.47 (m, 2H), 7.47-7.31 (m, 4H), 7.12 (d, J = 7.2 Hz, |
| | 1H), 6.97 (t, J = 8.5 Hz, 2H), 6.83-6.70 (m, 2H), 5.75 (d, J = 7.2 Hz, 1H), 4.97-4.84 (m, |
| | 1H), 4.31-4.19 (m, 1H), 4.03-3.92 (m, 2H), 3.70-3.63 (m, 2H), 3.61-3.51 (m, 6H), 3.51- |
| | 3.41 (m, 2H), 3.31-3.14 (m, 2H), 2.89-2.67 (m, 5H), 2.49-2.32 (m, 2H), 2.32-2.16 (m, |
| | 4H), 2.12-2.07 (m, 1H). |
| TDH-218 | 1H NMR (400 MHz, CDCl3) δ 9.14 (d, J = 7.7 Hz, 1H), 8.43-8.36 (m, 1H), 8.00 (s, 1H), |
| | 7.91 (s, 1H), 7.64-7.46 (m, 5H), 7.45-7.31 (m, 3H), 7.04-6.91 (m, 3H), 6.80-6.71 (m, |
| | 1H), 5.79-5.70 (m, 1H), 4.95-4.83 (m, 1H), 4.32-4.17 (m, 1H), 3.95-3.85 (m, 2H), 3.70- |
| | 3.61 (m, 2H), 3.61-3.53 (m, 6H), 3.52-3.45 (m, 2H), 3.28-3.16 (m, 2H), 2.88-2.66 (m, |
| | 5H), 2.43-2.30 (m, 2H), 2.28-2.17 (m, 4H), 2.10-2.02 (m, 1H). |
| TDH-221 | 1H NMR (500 MHz, Chloroform-d) δ 9.12 (s, 1H), 9.08 (s, 1H), 8.29 (s, 1H), 8.07 (s, 1H), |
| | 7.70 (d, J = 8.5 Hz, 1H), 7.68-7.61 (m, 4H), 7.47-7.41 (m, 3H), 7.30 (d, J = 2.3 Hz, 1H), |
| | 7.28-7.02 (m, 3H), 6.55 (s, 2H), 6.45 (s, 1H), 5.04-4.80 (m, 1H), 4.71-4.56 (m, 2H), 3.15- |
| | 3.04 (m, 2H), 2.98-2.77 (m, 2H), 2.67-2.40 (m, 1H), 2.35-2.22 (m, 6H), 2.18-2.05 (m, 6H), |
| | 1.96 (s, 2H), 1.60-1.10(m, 2H) |
| TDH-222 | 1H NMR (400 MHz, Chloroform-d) δ 9.12 (s, 1H), 9.03 (d, J = 5.2 Hz, 1H), 8.26-8.13 (m, |
| | 1H), 8.05 (s, 1H), 7.72-7.56 (m, 5H), 7.48-7.40 (m, 3H), 7.38 (d, J = 7.2 Hz, 1H), 7.20 (d, J = |
| | 8.4 Hz, 1H), 7.07 (t, J = 8.5 Hz, 2H), 6.52 (s, 2H), 6.45 (s, 1H), 5.02-4.93 (m, 1H), 4.73- |
| | 4.65 (m, 1H), 4.64-4.54 (m, 1H), 4.64-4.52 (m, 2H), 3.96-3.88 (m, 1H), 3.75 (s, 2H), 3.13- |
| | 3.02 (m, 2H), 2.95-2.92 (m, 2H), 2.90-2.67 (m, 2H), 2.66-2.56 (m, 1H), 2.35 (d, J = 6.8 Hz, |
| | 2H), 2.33-2.19 (m, 6H), 2.18-2.07 (m, 4H), 1.96-1.87 (m, 2H), 1.84-1.77 (m, 2H), 1.66- |
| | 1.52 (m, 2H), 1.19-1.09 (m, 2H). |
| TDH-223 | 1H NMR (400 MHz, Chloroform-d) δ 9.11 (s, 1H), 9.07 (s, 1H), 8.31 (s, 2H), 8.07 (s, 1H), |
| | 7.66-7.62 (m, 4H), 7.53 (t, J = 7.8 Hz, 2H), 7.45-7.42 (m, 2H), 7.24 (s, 1H), 7.15 (d, J = |
| | 7.4 Hz, 2H), 7.07 (t, J = 8.6 Hz, 2H), 6.82 (d, J = 8.4 Hz, 2H), 6.54 (s, 2H), 6.45 (s, 1H), |
| | 4.99-4.92 (m, 2H), 4.72-4.65 (m, 2H), 4.08-4.05 (m, 2H), 3.83-3.76 (m, 2H), 3.08-3.01 (m, |
| | 4H), 2.92-2.85 (m, 2H), 2.75-2.70 (m, 2H), 2.32-2.24 (m, 6H), 2.10-2.04 (m, 4H), 1.97- |
| | 1.93 (m, 2H), 1.25-1.20 (m, 2H). |
| TDH-224 | 1H NMR (400 MHz, Chloroform-d) δ 9.10 (s, 1H), 9.01 (d, J = 6.5 Hz, 1H), 8.17 (s, 2H), |
| | 8.05-8.03 (m, 1H), 7.65-7.60 (m, 4H), 7.44-7.39(m, 2H), 7.14-7.02 (m, 3H), 6.98-6.92 (m, |
| | 2H), 6.88-6.79 (m, 2H), 6.50 (s, 2H), 6.42 (s, 1H), 5.91 (s, 2H), 4.96-4.88 (m, 2H), 4.66- |
| | 4.64 (m, 2H), 3.97-3.94 (m, 2H), 3.80-3.74 (m, 2H), 3.04 (s, 4H), 3.77-3.74 (m, 2H), 2.76 |
| | (s, 2H), 2.27-2.20 (m, 6H), 2.19-2.11 (m, 4H), 2.04-2.01 (m, 2H), 1.84 (s, 2H), 1.21-1.15 |
| | (m, 2H). |
| TDH-225 | 1H NMR (400 MHz, Chloroform-d) δ 9.09 (d, J = 6.5 Hz, 2H), 8.51 (s, 1H), 8.04 (d, J = 2.3 |
| | Hz, 1H), 7.66-7.54 (m, 5H), 7.50-7.35 (m, 4H), 7.17 (d, J = 8.5 Hz, 1H), 7.10-6.99 (m, 2H), |
| | 6.54 (s, 2H), 6.43 (s, 1H), 5.05-4.88 (m, 1H), 4.66-4.47 (m, 1H), 3.82-3.69 (m, 2H), 3.12- |
| | 3.04 (m, 2H), 2.96-2.81 (m, 4H), 2.80-2.68 (m, 2H), 2.36-2.29 (m, 2H), 2.27-2.15 (m, 5H), |
| | 2.12-2.06 (m, 2H), 1.94-1.87 (m, 2H), 1.52-1.47(m, 2H). |
| TDH-226 | 1H NMR (400 MHz, Chloroform-d) δ 9.10 (d, J = 8.8 Hz, 2H), 8.50 (s, 1H), 8.08 (s, 1H), |
| | 7.72-7.60 (m, 5H), 7.52-7.47 (m, 3H), 7.16-6.99 (m, 4H), 6.65-6.50 (m, 2H), 6.45 (s, 1H), |
| | 5.02-4.94 (m, 1H), 4.66-4.54 (m, 1H), 4.02-3.90 (m, 2H), 3.04-2.97 (m, 2H), 2.94-2.83 (m, |
| | 2H), 2.95-2.83 (m, 2H), 2.82-2.73 (m, 2H), 2.34-2.29 (m, 2H), 2.29-2.19 (m, 5H), 2.15- |
| | 2.09 (m, 2H), 1.95-1.90 (m, 2H), 1.36-1.29 (m, 2H). |
| TDH-227 | 1H NMR (400 MHz, CDCl3) δ 8.95 (s, 1H), 8.50 (s, 1H), 8.00 (s, 1H), 7.88 (s, 1H), 7.68- |
| | 7.61 (m, 2H), 7.57-7.50 (m, 2H), 7.46-7.37 (m, 3H), 7.00 (t, J = 8.5 Hz, 2H), 6.44 (s, 1H), |
| | 6.36 (s, 2H), 4.98-4.88 (m, 1H), 3.85-3.63 (m, 2H), 3.63-3.45 (m, 2H), 2.51-2.37 (m, |
| | 2H), 1.47 (s, 9H). LC/MS (ESI) m/z 602 [M + H]+ |
| TDH-228 | 1H NMR (400 MHz, CDCl3) δ 8.92 (s, 0.5H), 8.88-8.87 (m, 0.5H), 8.52-8.46 (m, 1H), 8.00 |
| | (d, J = 7.2 Hz, 1H), 7.91-7.86 (m, 1H), 7.68-7.61 (m, 2H), 7.57-7.50 (m, 2H), 7.46-7.37 |
| | (m, 3H), 7.05-6.97 (m, 2H), 6.43 (s, 1H), 6.36 (s, 2H), 5.03-4.95 (m, 0.5H), 4.95-4.88 (m, |
| | 0.5H), 4.04-3.91 (m, 2H), 3.86-3.75 (m, 1H), 3.71-3.61 (m, 1H), 2.66-2.56 (m, 0.5H), |
| | 2.55-2.48 (m, 0.5H), 2.48-2.40 (m, 1H), 2.10 (d, J = 4.4 Hz, 2H), 2.08 (d, J = 4.0 Hz, 1H). |
| | LC/MS (ESI) m/z 544 [M + H]+ |
| TDH-229 | 1H NMR (400 MHz, CDCl3) δ 8.92 (s, 0.5H), 8.88-8.87 (m, 0.5H), 8.52-8.47 (m, 1H), 8.00 |
| | (d, J = 7.2 Hz, 1H), 7.91-7.86 (m, 1H), 7.68-7.61 (m, 2H), 7.57-7.49 (m, 2H), 7.46-7.37 |
| | (m, 3H), 7.05-6.97 (m, 2H), 6.43 (s, 1H), 6.36 (s, 2H), 5.03-4.96 (m, 0.5H), 4.96-4.87 (m, |
| | 0.5H), 4.04-3.90 (m, 2H), 3.85-3.73 (m, 1H), 3.72-3.60 (m, 1H), 2.65-2.56 (m, 0.5H), |
| | 2.55-2.49 (m, 0.5H), 2.49-2.40 (m, 1H), 2.10 (d, J = 4.5 Hz, 2H), 2.08 (d, J = 4.0 Hz, 1H). |
| | LC/MS (ESI) m/z 544 [M + H]+ |
| TDH-230 | 1H NMR (500 MHz, CDCl3) δ 9.06 (s, 1H), 8.84 (d, J = 2.4 Hz, 1H), 8.69 (s, 1H), 8.46 (s, |
| | 1H), 8.07 (dd, J = 8.9, 2.4 Hz, 1H), 7.65 (d, J = 7.5 Hz, 2H), 7.62-7.55 (m, 3H), 7.47-7.37 |
| | (m, 4H), 7.20 (d, J = 8.5 Hz, 1H), 7.03-6.96 (m, 2H), 6.76 (d, J = 8.9 Hz, 1H), 6.50-6.37 |
| | (m, 3H), 5.02-4.96 (m, 1H), 4.73-4.65 (m, 1H), 4.03-3.95 (m, 1H), 3.90-3.79 (m, 2H), |
| | 3.74-3.62 (m, 4H), 3.15-3.02 (m, 3H), 2.95-2.88 (m, 1H), 2.87-2.71 (m, 3H), 2.66-2.54 |
| | (m, 5H), 2.34-2.25 (m, 2H), 2.18-2.06 (m, 3H), 2.01-1.94 (m, 1H), 1.92-1.82 (m, 4H), |
| | 1.24-1.09 (m, 2H). |
| TDH-231 | 1H NMR (500 MHz, CDCl3) δ 9.05 (s, 1H), 8.86-8.82 (m, 1H), 8.69 (s, 1H), 8.34 (s, 1H), |
| | 8.10-8.05 (m, 1H), 7.73-7.69 (m, 1H), 7.67-7.63 (m, 2H), 7.61-7.55 (m, 2H), 7.48-7.38 |
| | (m, 3H), 7.32-7.30 (m, 1H), 7.11-7.06 (m, 1H), 7.03-6.97 (m, 2H), 6.76 (d, J = 8.9 Hz, |
| | 1H), 6.48-6.33 (m, 3H), 5.00-4.93 (m, 1H), 4.72-4.63 (m, 1H), 4.05-3.94 (m, 3H), 3.76- |
| | 3.62 (m, 4H), 3.18-3.04 (m, 3H), 2.95-2.88 (m, 1H), 2.88-2.71 (m, 3H), 2.68-2.53 (m, |
| | 5H), 2.31 (d, J = 7.0 Hz, 2H), 2.20-2.13 (m, 1H), 2.04-1.92 (m, 3H), 1.92-1.84 (m, 4H), |
| | 1.24-1.10 (m, 2H). |
| TDH-232 | 1H NMR (500 MHz, CDCl3) δ 9.04 (s, 1H), 8.82 (d, J = 2.4 Hz, 1H), 8.67 (s, 1H), 8.35- |
| | 8.27 (m, 1H), 8.08-8.03 (m, 1H), 7.63 (d, J = 7.4 Hz, 2H), 7.59-7.54 (m, 2H), 7.51 (t, J = |
| | 7.8 Hz, 1H), 7.45-7.36 (m, 3H), 7.25-7.21 (m, 1H), 7.14 (d, J = 7.1 Hz, 1H), 6.98 (t, J = |
| | 8.4 Hz, 2H), 6.80 (d, J = 8.4 Hz, 1H), 6.74 (d, J = 8.8 Hz, 1H), 6.47-6.29 (m, 3H), 4.97- |
| | 4.91 (m, 1H), 4.71-4.63 (m, 1H), 4.04 (d, J = 4.4 Hz, 2H), 3.82-3.75 (m, 1H), 3.72-3.60 |
| | (m, 4H), 3.17-3.08 (m, 1H), 2.93-2.79 (m, 2H), 2.78-2.67 (m, 2H), 2.64-2.50 (m, 4H), |
| | 2.34-2.25 (m, 2H), 2.17-2.10 (m, 1H), 2.02-1.95 (m, 1H), 1.93-1.82 (m, 2H), 1.23-1.14 |
| | (m, 2H). |
| TDH-233 | 1H NMR (500 MHz, CDCl3) δ 9.03 (s, 1H), 8.82 (d, J = 2.5 Hz, 1H), 8.67 (s, 1H), 8.28 (s, |
| | 1H), 8.05 (dd, J = 8.9, 2.5 Hz, 1H), 7.67-7.61 (m, 3H), 7.60-7.53 (m, 2H), 7.45-7.36 (m, |
| | 3H), 7.02-6.94 (m, 3H), 6.84 (dd, J = 8.3, 2.3 Hz, 1H), 6.74 (d, J = 8.9 Hz, 1H), 6.46-6.30 |
| | (m, 3H), 5.97-5.91 (m, 1H), 4.98-4.91 (m, 1H), 4.71-4.61 (m, 1H), 4.01-3.92 (m, 2H), |
| | 3.83-3.75 (m, 1H), 3.73-3.60 (m, 4H), 3.19-3.08 (m, 1H), 2.93-2.86 (m, 1H), 2.86-2.69 |
| | (m, 3H), 2.64-2.51 (m, 4H), 2.29 (d, J = 7.0 Hz, 2H), 2.18-2.10 (m, 1H), 2.06-1.97 (m, |
| | 1H), 1.96-1.84 (m, 2H), 1.24-1.12 (m, 2H). |
| TDH-234 | 1H NMR (300 MHz, MeOH-d4) δ 8.61 (m, 1H), 8.25 (m, 1H), 8.01 (m, 1H), 7.74 (d, J = 7.1 |
| | Hz, 2H), 7.67-7.54 (m, 3H), 7.46 (q, J = 8.2, 7.4 Hz, 3H), 7.15 (d, J = 8.5 Hz, 1H), 7.11- |
| | 7.01 (m, 3H), 6.35 (s, 1H), 5.06 (dd, J = 12.1, 5.6 Hz, 1H), 4.23 (m, 1H), 3.53 (m, 4H), |
| | 3.04 (m, 4H), 2.89-2.68 (m, 5H), 2.22 (m, 12H), 1.72 (d, J = 12.8 Hz, 2H), 1.53 (m, 2H), |
| | 1.31 (m, 1H). |
| TDH-235 | 1H NMR (400 MHz, MeOH-d4) δ 8.60 (d, J = 9.2 Hz, 1H), 8.24 (d, J = 17.2 Hz, 1H), 8.01 |
| | (d, J = 11.4 Hz, 1H), 7.79-7.68 (m, 2H), 7.58 (m, 3H), 7.52-7.41 (m, 3H), 7.13-7.05 (m, |
| | 3H), 6.34 (s, 1H), 5.07 (dd, J = 12.5, 5.4 Hz, 1H), 4.24 (m, 1H), 3.40 (m, 4H), 3.08 (d, J = |
| | 12.7 Hz, 4H), 2.91 (d, J = 11.1 Hz, 2H), 2.85-2.68 (m, 3H), 2.32 (d, J = 7.0 Hz, 2H), 2.23 |
| | (d, J = 12.7 Hz, 4H), 2.18-2.00 (m, 8H), 1.89 (p, J = 6.6 Hz, 2H), 1.78 (d, J = 13.2 Hz, |
| | 2H), 1.35 (m, 1H). |
| TDH-236 | 1H NMR (400 MHz, MeOH-d4) δ 8.60 (m, 1H), 8.24 (m, 1H), 8.01 (m, 1H), 7.77-7.68 (m, |
| | 2H), 7.66 (d, J = 7.9 Hz, 1H), 7.60 (dd, J = 9.0, 4.9 Hz, 2H), 7.46 (m, , 3H), 7.38 (d, J = 7.1 |
| | Hz, 1H), 7.35 (d, J = 8.5 Hz, 1H), 7.07 (t, J = 8.7 Hz, 2H), 6.35 (s, 1H), 5.12 (dd, J = 12.6, |
| | 5.4 Hz, 1H), 4.26 (m, 1H), 3.99-3.89 (m, 2H), 3.79-3.71 (m, 3H), 3.09 (m, 4H), 3.07 (m, |
| | 1H), 2.96 (d, J = 11.5 Hz, 2H), 2.89-2.83 (m, 1H), 2.77 (d, J = 14.2 Hz, 2H), 2.35 (d, J = |
| | 7.0 Hz, 2H), 2.26 (d, J = 11.6 Hz, 2H), 2.24-2.20 (m, 2H), 2.18-2.11 (m, 5H), 2.04 (m, |
| | 1H), 2.01 (m, 1H), 1.84 (m, 3H), 1.36 (m, 3H). |
| TDH-237 | 1H NMR (400 MHz, MeOH-d4) δ 8.60 (m, 1H), 8.24 (m, 1H), 8.01 (m, 1H), 7.92 (s, 1H), |
| | 7.73 (t, J = 6.6 Hz, 3H), 7.60 (dd, J = 8.9, 4.8 Hz, 2H), 7.46 (m, 4H), 7.36 (d, J = 8.5 Hz, |
| | 1H), 7.07 (t, J = 8.7 Hz, 2H), 6.35 (s, 1H), 5.13 (dd, J = 12.5, 5.3 Hz, 2H), 4.23 (m, 1H), |
| | 3.91-3.76 (m, 4H), 3.41 (m, 4H), 3.22 (m, 1H), 3.05 (t, J = 13.1 Hz, 4H), 2.95-2.84 (m, |
| | 2H), 2.81-2.68 (m, 3H), 2.32 (d, J = 7.1 Hz, 2H), 2.21 (m, 3H), 2.17-2.06 (m, 5H), 2.04 |
| | (m, 3H), 1.84 (d, J = 13.0 Hz, 2H). |
| TDH-238 | 1H NMR (400 MHz, MeOH-d4) δ 8.60 (m, 1H), 8.24 (m, 1H), 8.01 (m, 1H), 7.73 (d, J = 7.4 |
| | Hz, 2H), 7.69-7.51 (m, 4H), 7.47 (m, 2H), 7.17 (s, 1H), 7.08 (m, 3H), 6.35 (s, 1H), 5.11- |
| | 5.02 (m, 1H), 4.41 (d, J = 13.5 Hz, 2H), 4.24 (m, 1H), 4.09 (d, J = 13.7 Hz, 2H), 3.89 (m, |
| | 1H), 3.51 (m, 1H), 3.05 (m, 6H), 2.77 (m, 4H), 2.28 (m, 6H), 2.14 (m, 7H), 2.05 (d, J = 9.4 |
| | Hz, 2H), 1.85 (m, 2H), 1.46 (m, 3H). |
| TDH-239 | ¹H NMR (400 MHz, DMSO-d₆) δ 11.09 (s, 1H), 10.69 (s, 1H), 8.69 (s, 1H), 8.29 (s, 1H), |
| | 7.92 (s, 1H), 7.80-7.57 (m, 5H), 7.55-7.40 (m, 3H), 7.40-7.22 (m, 4H), 7.20-7.11 (m, |
| | 2H), 6.26 (s, 1H), 5.16-5.02 (m, 1H), 4.29-4.13 (m, 1H), 3.75-3.69 (m, 2H), 3.55-3.48 |
| | (m, 3H), 3.09-2.93 (m, 4H), 2.92-2.78 (m, 2H), 2.63-2.54 (m, 2H), 2.45-2.30 (m, 8H), |
| | 2.14-1.92 (m, 8H), 1.84-1.68 (m, 4H), 1.68-1.56 (m, 2H). |
| TDH-240 | ¹H NMR (400 MHz, DMSO-d₆) δ 11.07 (s, 1H), 10.70 (s, 1H), 8.69 (s, 1H), 8.29 (s, 1H), |
| | 7.92 (s, 1H), 7.78-7.71 (m, 2H), 7.70-7.59 (m, 3H), 7.53-7.46 (m, 2H), 7.46-7.40 (m, |
| | 1H), 7.34-7.21 (m, 4H), 7.21-7.11 (m, 2H), 6.26 (s, 1H), 5.16-5.03 (m, 1H), 4.25-4.15 |
| | (m, 1H), 4.11-4.01 (m, 2H), 3.61-3.52 (m, 3H), 3.13-3.02 (m, 3H), 2.99-2.91 (m, 3H), |
| | 2.61-2.54 (m, 2H), 2.40-2.29 (m, 8H), 2.09-1.93 (m, 8H), 1.72-1.58 (m, 6H). |
| TDH-241 | ¹H NMR (400 MHz, DMSO-d₆) δ 11.10 (s, 1H), 10.71 (s, 1H), 8.70 (s, 1H), 8.29 (s, 1H), |
| | 7.93 (s, 1H), 7.78-7.71 (m, 2H), 7.68-7.58 (m, 3H), 7.55-7.46 (m, 2H), 7.46-7.39 (m, |
| | 1H), 7.35-7.25 (m, 2H), 7.18-7.12 (m, 2H), 7.12-7.04 (m, 3H), 6.26 (s, 1H), 5.12-5.02 |
| | (m, 1H), 4.28-4.17 (m, 3H), 3.54-3.51 (m, 3H), 3.09-2.98 (m, 2H), 2.94-2.84 (m, 1H), |
| | 2.62-2.56 (m, 2H), 2.46-2.33 (m, 8H), 2.26-2.12 (m, 2H), 2.08-1.91 (m, 6H), 1.73-1.62 |
| | (m, 2H). |
| TDH-242 | ¹H NMR (400 MHz, DMSO-d₆) δ 11.06 (s, 1H), 10.68 (s, 1H), 8.70 (s, 1H), 8.29 (s, 1H), |
| | 7.93 (s, 1H), 7.77-7.70 (m, 2H), 7.67-7.54 (m, 3H), 7.54-7.38 (m, 3H), 7.34-7.24 (m, |
| | 2H), 7.20-7.07 (m, 4H), 7.06-6.98 (m, 1H), 6.24 (s, 1H), 5.09-4.99 (m, 1H), 4.29-4.17 |
| | (m, 1H), 4.17-4.09 (m, 2H), 3.53-3.50 (m, 3H), 3.15-2.97 (m, 2H), 2.93-2.81 (m, 1H), |
| | 2.63-2.54 (m, 2H), 2.46-2.32 (m, 8H), 2.27-2.11 (m, 2H), 2.11-1.92 (m, 6H), 1.73-1.59 |
| | (m, 2H). |
| TDH-243 | ¹H NMR (400 MHz, DMSO-d₆) δ 11.11 (s, 1H), 10.70 (s, 1H), 8.69 (s, 1H), 8.29 (s, 1H), |
| | 7.92 (s, 1H), 7.82-7.70 (m, 3H), 7.67-7.59 (m, 2H), 7.54-7.39 (m, 4H), 7.37-7.26 (m, |
| | 3H), 7.22-7.11 (m, 2H), 6.26 (s, 1H), 5.18 (s, 2H), 5.16-5.06 (m, 1H), 4.27-4.15 (m, 1H), |
| | 3.47-3.44 (m, 3H), 3.05-2.95 (m, 2H), 2.93-2.83 (m, 1H), 2.64-2.54 (m, 2H), 2.46-2.40 |
| | (m, 2H), 2.40-2.31 (m, 6H), 2.12-1.92 (m, 8H), 1.70-1.57 (m, 2H). |
| TDH-244 | ¹H NMR (400 MHz, DMSO-d₆) δ 11.11 (s, 1H), 10.65 (s, 1H), 8.70 (s, 1H), 8.29 (s, 1H), |
| | 7.92 (s, 1H), 7.88-7.81 (m, 1H), 7.77-7.70 (m, 2H), 7.67-7.59 (m, 2H), 7.54-7.46 (m, |
| | 2H), 7.46-7.40 (m, 2H), 7.39-7.33 (m, 1H), 7.33-7.26 (m, 2H), 7.21-7.12 (m, 2H), 6.25 |
| | (s, 1H), 5.17-5.08 (m, 3H), 4.25-4.14 (m, 1H), 3.47-3.45 (m, 3H), 2.99-2.92 (m, 2H), |
| | 2.91-2.83 (m, 1H), 2.64-2.54 (m, 2H), 2.47-2.40 (m, 2H), 2.40-2.32 (m, 6H), 2.10-1.93 |
| | (m, 8H), 1.69-1.58 (m, 2H). |
| TDH-245 | ¹H NMR (400 MHz, DMSO-d₆) δ 11.09 (s, 1H), 10.65 (s, 1H), 8.70 (s, 1H), 8.30 (s, 1H), |
| | 7.93 (s, 1H), 7.76-7.68 (m, 3H), 7.66-7.56 (m, 2H), 7.53-7.41 (m, 3H), 7.38-7.33 (m, |
| | 2H), 7.32-7.25 (m, 2H), 7.20-7.12 (m, 2H), 6.24 (s, 1H), 5.16-5.06 (m, 1H), 4.30-4.16 |
| | (m, 1H), 3.65-3.47 (m, 3H), 3.09-3.00 (m, 2H), 2.92-2.82 (m, 1H), 2.66-2.56 (m, 6H), |
| | 2.47-2.36 (m, 4H), 2.21-1.94 (m, 8H), 1.76-1.64 (m, 2H). |
| TDH-246 | ¹H NMR (400 MHz, DMSO-d₆) δ 11.08 (s, 1H), 10.65 (s, 1H), 8.70 (s, 1H), 8.30 (s, 1H), |
| | 7.93 (s, 1H), 7.77-7.71 (m, 2H), 7.71-7.66 (m, 1H), 7.66-7.56 (m, 2H), 7.54-7.47 (m, |
| | 2H), 7.47-7.40 (m, 1H), 7.37-7.33 (m, 1H), 7.33-7.25 (m, 3H), 7.21-7.12 (m, 2H), 6.25 |
| | (s, 1H), 5.14-5.03 (m, 1H), 4.21 (s, 1H), 3.47-3.44 (m, 3H), 3.09-2.96 (m, 2H), 2.95-2.80 |
| | (m, 1H), 2.69-2.54 (m, 6H), 2.45-2.31 (m, 4H), 2.17-1.93 (m, 8H), 1.73-1.63 (m, 2H). |
| TDH-247 | ¹H NMR (400 MHz, DMSO-d₆) δ 11.08 (s, 1H), 10.62 (s, 1H), 8.69 (s, 1H), 8.29 (s, 1H), |
| | 7.92 (s, 1H), 7.76-7.55 (m, 5H), 7.52-7.40 (m, 3H), 7.36-7.25 (m, 4H), 7.21-7.12 (m, |
| | 2H), 6.23 (s, 1H), 5.16-5.04 (m, 1H), 4.24-4.12 (m, 1H), 3.92-3.76 (m, 2H), 3.74-3.67 |
| | (m, 2H), 3.62-3.47 (m, 4H), 3.14-3.06 (m, 1H), 3.06-2.91 (m, 5H), 2.91-2.77 (m, 2H), |
| | 2.64-2.54 (m, 4H), 2.24-2.15 (m, 2H), 2.09-1.98 (m, 4H), 1.98-1.90 (m, 2H), 1.80-1.68 |
| | (m, 4H), 1.50-1.40 (m, 1H), 1.38-1.30 (m, 1H). |
| TDH-248 | ¹H NMR (400 MHz, DMSO-d₆) δ 11.08 (s, 1H), 10.63 (s, 1H), 8.69 (s, 1H), 8.29 (s, 1H), |
| | 7.92 (s, 1H), 7.76-7.70 (m, 2H), 7.70-7.58 (m, 3H), 7.54-7.47 (m, 2H), 7.47-7.40 (m, |
| | 1H), 7.33-7.29 (m, 2H), 7.28-7.21 (m, 2H), 7.21-7.11 (m, 2H), 6.24 (s, 1H), 5.17-5.03 |
| | (m, 1H), 4.25-4.13 (m, 1H), 4.13-3.96 (m, 2H), 3.86-3.74 (m, 2H), 3.63-3.51 (m, 4H), |
| | 3.14-2.95 (m, 7H), 2.92-2.80 (m, 1H), 2.64-2.54 (m, 4H), 2.26-2.15 (m, 2H), 2.04-1.97 |
| | (m, 4H), 1.84-1.74 (m, 2H), 1.71-1.56 (m, 4H), 1.49-1.41 (m, 1H), 1.36-1.28 (m, 1H). |
| TDH-249 | ¹H NMR (400 MHz, DMSO-d₆) δ 11.10 (s, 1H), 10.63 (s, 1H), 8.69 (s, 1H), 8.29 (s, 1H), |
| | 7.92 (s, 1H), 7.75-7.68 (m, 2H), 7.66-7.57 (m, 3H), 7.53-7.40 (m, 3H), 7.34-7.24 (m, |
| | 2H), 7.20-7.03 (m, 5H), 6.24 (s, 1H), 5.13-5.02 (m, 1H), 4.30-4.12 (m, 3H), 3.93-3.84 |
| | (m, 1H), 3.69-3.64 (m, 1H), 3.59-3.55 (m, 2H), 3.26-3.17 (m, 2H), 3.06-2.97 (m, 2H), |
| | 2.95-2.85 (m, 1H), 2.65-2.53 (m, 4H), 2.27-2.14 (m, 2H), 2.07-1.80 (m, 8H), 1.57-1.46 |
| | (m, 1H), 1.46-1.36 (m, 1H). |
| TDH-250 | ¹H NMR (400 MHz, DMSO-d₆) δ 11.06 (s, 1H), 10.62 (s, 1H), 8.69 (s, 1H), 8.29 (s, 1H), |
| | 7.92 (s, 1H), 7.77-7.70 (m, 2H), 7.66-7.55 (m, 3H), 7.54-7.41 (m, 3H), 7.36-7.25 (m, |
| | 2H), 7.21-7.11 (m, 3H), 7.11-6.99 (m, 2H), 6.24 (s, 1H), 5.10-5.00 (m, 1H), 4.24-4.10 |
| | (m, 3H), 3.92-3.82 (m, 1H), 3.77-3.67 (m, 1H), 3.63-3.50 (m, 2H), 3.29-3.14 (m, 2H), |
| | 3.07-2.95 (m, 2H), 2.95-2.83 (m, 1H), 2.66-2.53 (m, 4H), 2.26-2.14 (m, 2H), 2.09-1.78 |
| | (m, 8H), 1.57-1.45 (m, 1H), 1.45-1.33 (m, 1H). |
| TDH-251 | ¹H NMR (400 MHz, DMSO-d₆) δ 11.11 (s, 1H), 10.63 (s, 1H), 8.69 (s, 1H), 8.29 (s, 1H), |
| | 7.92 (s, 1H), 7.81-7.70 (m, 3H), 7.67-7.58 (m, 2H), 7.55-7.40 (m, 4H), 7.37-7.25 (m, |
| | 3H), 7.21-7.12 (m, 2H), 6.24 (s, 1H), 5.18 (s, 2H), 5.16-5.06 (m, 1H), 4.24-4.13 (m, 1H), |
| | 3.84-3.74 (m, 1H), 3.65-3.53 (m, 4H), 3.27-3.20 (m, 1H), 3.19-3.11 (m, 1H), 3.07-2.96 |
| | (m, 2H), 2.96-2.84 (m, 1H), 2.65-2.53 (m, 4H), 2.27-2.14 (m, 2H), 2.06-1.90 (m, 6H), |
| | 1.85-1.77 (m, 1H), 1.62-1.50 (m, 1H), 1.46-1.34 (m, 1H). |
| TDH-252 | ¹H NMR (400 MHz, DMSO-d₆) δ 11.11 (s, 1H), 10.62 (s, 1H), 8.69 (s, 1H), 8.28 (s, 1H), |
| | 7.92 (s, 1H), 7.86-7.80 (m, 1H), 7.75-7.69 (m, 2H), 7.66-7.58 (m, 2H), 7.53-7.46 (m, |
| | 2H), 7.46-7.40 (m, 2H), 7.37-7.32 (m, 1H), 7.32-7.25 (m, 2H), 7.20-7.12 (m, 2H), 6.23 |
| | (s, 1H), 5.22-5.06 (m, 3H), 4.25-4.12 (m, 1H), 3.87-3.76 (m, 1H), 3.67-3.55 (m, 4H), |
| | 3.27-3.19 (m, 1H), 3.19-3.10 (m, 1H), 3.04-2.95 (m, 2H), 2.95-2.81 (m, 1H), 2.64-2.54 |
| | (m, 4H), 2.25-2.14 (m, 2H), 2.08-1.90 (m, 6H), 1.85-1.75 (m, 1H), 1.59-1.47 (m, 1H), |
| | 1.44-1.32 (m, 1H). |
| TDH-253 | ¹H NMR (400 MHz, DMSO-d₆) δ 11.09 (s, 1H), 10.62 (s, 1H), 8.69 (s, 1H), 8.29 (s, 1H), |
| | 7.92 (s, 1H), 7.77-7.59 (m, 5H), 7.55-7.40 (m, 3H), 7.37-7.25 (m, 4H), 7.22-7.12 (m, |
| | 2H), 6.24 (s, 1H), 5.15-5.06 (m, 1H), 4.24-4.13 (m, 1H), 3.64-3.59 (m, 2H), 3.59-3.47 |
| | (m, 3H), 3.15-2.98 (m, 4H), 2.95-2.82 (m, 1H), 2.69-2.55 (m, 4H), 2.27-2.15 (m, 2H), |
| | 2.09-1.90 (m, 7H), 1.72-1.59 (m, 2H). |
| TDH-254 | ¹H NMR (400 MHz, DMSO-d₆) δ 11.08 (s, 1H), 10.62 (s, 1H), 8.69 (s, 1H), 8.29 (s, 1H), |
| | 7.92 (s, 1H), 7.77-7.70 (m, 2H), 7.70-7.58 (m, 3H), 7.54-7.39 (m, 3H), 7.35-7.23 (m, |
| | 4H), 7.21-7.12 (m, 2H), 6.24 (s, 1H), 5.14-5.02 (m, 1H), 4.24-4.10 (m, 1H), 3.84-3.76 |
| | (m, 2H), 3.62-3.55 (m, 3H), 3.30-3.24 (m, 2H), 3.03-2.95 (m, 2H), 2.95-2.85 (m, 1H), |
| | 2.62-2.54 (m, 4H), 2.25-2.15 (m, 2H), 2.06-1.98 (m, 4H), 1.97-1.89 (m, 3H), 1.58-1.48 |
| | (m, 2H). |
| TDH-255 | 1H NMR (500 MHz, CDCl3) δ 9.05 (s, 1H), 8.81 (d, J = 2.4 Hz, 1H), 8.70-8.61 (m, 2H), |
| | 8.07-8.01 (m, 1H), 7.68 (t, J = 7.9 Hz, 1H), 7.62 (d, J = 7.5 Hz, 2H), 7.59-7.53 (m, 2H), |
| | 7.50 (d, J = 7.3 Hz, 1H), 7.44-7.36 (m, 3H), 7.35-7.30 (m, 1H), 6.97 (t, J = 8.5 Hz, 2H), |
| | 6.72 (d, J = 8.9 Hz, 1H), 6.51-6.33 (m, 3H), 5.05-4.93 (m, 3H), 4.57-4.49 (m, 1H), 4.08- |
| | 3.99 (m, 1H), 3.71-3.57 (m, 4H), 3.16-3.07 (m, 1H), 2.93-2.86 (m, 1H), 2.84-2.62 (m, |
| | 3H), 2.61-2.47 (m, 4H), 2.30-2.20 (m, 2H), 2.17-2.10 (m, 1H), 1.96-1.90 (m, 1H), 1.88- |
| | 1.80 (m, 2H), 1.22-1.06 (m, 2H). |
| TDH-256 | 1H NMR (500 MHz, CDCl3) δ 9.02 (s, 1H), 8.82 (s, 1H), 8.67 (s, 1H), 8.40 (s, 1H), 8.08- |
| | 8.02 (m, 1H), 7.81 (d, J = 8.3 Hz, 1H), 7.62 (d, J = 7.4 Hz, 2H), 7.59-7.53 (m, 2H), 7.45- |
| | 7.38 (m, 3H), 7.38-7.34 (m, 1H), 7.33-7.29 (m, 1H), 6.98 (t, J = 8.4 Hz, 2H), 6.74 (d, J = |
| | 8.9 Hz, 1H), 6.48-6.32 (m, 3H), 5.00-4.92 (m, 1H), 4.91-4.81 (m, 2H), 4.62-4.53 (m, 1H), |
| | 3.92-3.83 (m, 1H), 3.71-3.60 (m, 4H), 3.18-3.09 (m, 1H), 2.94-2.88 (m, 1H), 2.87-2.65 |
| | (m, 3H), 2.62-2.51 (m, 4H), 2.28 (d, J = 6.9 Hz, 2H), 2.19-2.11 (m, 1H), 2.01-1.94 (m, |
| | 1H), 1.92-1.85 (m, 2H), 1.23-1.10 (m, 2H). |
| TDH-257 | 1H NMR (500 MHz, CDCl3) δ 9.24 (s, 1H), 9.04 (s, 1H), 8.80-8.75 (m, 1H), 8.63 (s, 1H), |
| | 8.05-7.99 (m, 1H), 7.66-7.60 (m, 2H), 7.58-7.49 (m, 3H), 7.44-7.36 (m, 3H), 7.34-7.30 |
| | (m, 1H), 7.12 (d, J = 8.4 Hz, 1H), 6.99-6.93 (m, 2H), 6.71 (dd, J = 8.9, 3.0 Hz, 1H), 6.55- |
| | 6.47 (m, 1H), 6.48-6.36 (m, 3H), 4.93-4.86 (m, 1H), 3.78-3.67 (m, 8H), 3.66-3.62 (m, |
| | 4H), 3.58 (t, J = 4.9 Hz, 2H), 3.49 (q, J = 5.1 Hz, 2H), 2.97-2.87 (m, 2H), 2.87-2.61 (m, |
| | 9H), 2.39-2.31 (m, 1H), 2.11-2.05 (m, 1H), 2.03-1.97 (m, 4H). |
| TDH-258 | 1H NMR (500 MHz, CDCl3) δ 9.03 (s, 1H), 8.83-8.76 (m, 2H), 8.65-8.61 (m, 1H), 8.04 |
| | (dd, J = 8.9, 2.5 Hz, 1H), 7.66-7.58 (m, 3H), 7.57-7.52 (m, 2H), 7.44-7.35 (m, 3H), 7.19 |
| | (t, J = 2.5 Hz, 1H), 7.00-6.92 (m, 3H), 6.72 (d, J = 8.7 Hz, 1H), 6.62 (s, 1H), 6.51-6.38 |
| | (m, 3H), 4.94-4.86 (m, 1H), 3.94-3.84 (m, 2H), 3.74-3.67 (m, 6H), 3.67-3.64 (m, 4H), |
| | 3.59 (t, J = 5.0 Hz, 2H), 3.51-3.46 (m, 2H), 2.97-2.88 (m, 2H), 2.88-2.73 (m, 3H), 2.73- |
| | 2.64 (m, 6H), 2.43-2.34 (m, 1H), 2.14-2.06 (m, 1H), 2.05-1.97 (m, 2H), 1.88-1.77 (m, 2H). |
| TDH-259 | 1H NMR (500 MHz, CDCl3) δ 9.38 (s, 1H), 9.06 (s, 1H), 8.82-8.76 (m, 1H), 8.65 (s, 1H), |
| | 8.01 (dd, J = 8.9, 2.5 Hz, 1H), 7.65-7.60 (m, 2H), 7.59-7.54 (m, 2H), 7.53-7.48 (m, 1H), |
| | 7.45-7.36 (m, 3H), 7.18 (d, J = 7.1 Hz, 1H), 7.12-7.05 (m, 1H), 7.00-6.94 (m, 2H), 6.84- |
| | 6.79 (m, 1H), 6.79-6.73 (m, 1H), 6.72 (d, J = 8.9 Hz, 1H), 6.43 (s, 3H), 4.87-4.80 (m, 1H), |
| | 3.98 (d, J = 6.1 Hz, 2H), 3.77-3.67 (m, 4H), 3.67-3.62 (m, 2H), 3.59-3.55 (m, 4H), 3.55- |
| | 3.50 (m, 3H), 3.48-3.40 (m, 1H), 2.86-2.74 (m, 2H), 2.74-2.61 (m, 6H), 2.12-2.05 (m, 1H) |
| TDH-260 | 1H NMR (500 MHz, CDCl3) δ 9.06-9.00 (m, 1H), 8.80-8.75 (m, 1H), 8.68-8.64 (m, 1H), |
| | 8.07-8.01 (m, 1H), 7.66-7.60 (m, 2H), 7.58-7.52 (m, 3H), 7.45-7.36 (m, 3H), 7.13 (s, |
| | 1H), 7.00-6.92 (m, 3H), 6.78-6.71 (m, 2H), 6.47-6.32 (m, 3H), 5.87-5.81 (m, 1H), 4.88- |
| | 4.82 (m, 1H), 3.90 (d, J = 5.3 Hz, 2H), 3.75-3.64 (m, 8H), 3.61-3.59 (m, 2H), 3.59-3.56 |
| | (m, 2H), 3.54-3.50 (m, 2H), 2.85-2.79 (m, 1H), 2.78-2.62 (m, 8H), 2.09-2.05 (m, 1H). |
| TDH-261 | 1H NMR (500 MHz, CDCl3) δ 9.02 (d, J = 2.4 Hz, 1H), 8.80-8.77 (m, 1H), 8.65 (s, 1H), |
| | 8.05-8.00 (m, 1H), 7.77-7.71 (m, 2H), 7.65-7.60 (m, 2H), 7.57-7.52 (m, 3H), 7.44-7.37 |
| | (m, 3H), 7.17 (d, J = 8.4 Hz, 1H), 7.00-6.94 (m, 2H), 6.71 (d, J = 8.9 Hz, 1H), 6.42 (d, J = |
| | 2.3 Hz, 1H), 6.37 (s, 2H), 4.96-4.90 (m, 1H), 4.68-4.60 (m, 2H), 3.76-3.61 (m, 13H), |
| | 3.57-3.49 (m, 1H), 2.90-2.79 (m, 2H), 2.79-2.64 (m, 7H), 2.17-2.10 (m, 1H). |
| TDH-262 | 1H NMR (500 MHz, CDCl3) δ 9.04 (d, J = 4.0 Hz, 1H), 8.93 (d, J = 27.5 Hz, 1H), 8.83- |
| | 8.77 (m, 1H), 8.66 (d, J = 1.3 Hz, 1H), 8.06-7.99 (m, 1H), 7.78 (dd, J = 8.3, 2.2 Hz, 1H), |
| | 7.66-7.60 (m, 2H), 7.59-7.51 (m, 2H), 7.45-7.35 (m, 4H), 7.26-7.23 (m, 1H), 7.18-7.14 |
| | (m, 1H), 6.99-6.93 (m, 2H), 6.70 (dd, J = 8.9, 2.2 Hz, 1H), 6.50-6.36 (m, 3H), 4.93-4.86 |
| | (m, 1H), 4.62 (s, 2H), 3.71-3.61 (m, 12H), 3.61-3.56 (m, 2H), 2.88-2.72 (m, 2H), 2.72- |
| | 2.62 (m, 7H), 2.12-2.06 (m, 1H). |
| TDH-263 | 1H NMR (500 MHz, CDCl3) δ 9.03 (s, 1H), 8.56-8.50 (m, 1H), 8.09 (d, J = 6.8 Hz, 1H), |
| | 7.98 (s, 1H), 7.70-7.62 (m, 2H), 7.62-7.49 (m, 3H), 7.47-7.38 (m, 3H), 7.20 (d, J = 7.2 |
| | Hz, 1H), 7.16-7.06 (m, 1H), 7.04-6.96 (m, 2H), 6.87-6.79 (m, 1H), 6.79-6.71 (m, 1H), |
| | 6.66 (s, 2H), 6.48 (d, J = 4.7 Hz, 1H), 5.01 (s, 1H), 4.94-4.82 (m, 1H), 4.03-3.93 (m, 2H), |
| | 3.68-3.58 (m, 2H), 3.55-3.43 (m, 8H), 3.19-3.00 (m, 3H), 2.89-2.62 (m, 6H), 2.57-2.48 |
| | (m, 1H), 2.28-2.18 (m, 1H), 2.17-2.09 (m, 1H). |
| TDH-264 | 1H NMR (500 MHz, CDCl3) δ 9.08 (s, 1H), 8.55-8.48 (m, 1H), 8.11 (s, 1H), 7.97 (s, 1H), |
| | 7.66 (d, J = 7.3 Hz, 2H), 7.62-7.54 (m, 3H), 7.47-7.38 (m, 3H), 7.05-6.97 (m, 2H), 6.93 |
| | (s, 1H), 6.79-6.72 (m, 1H), 6.59 (s, 2H), 6.49-6.44 (m, 1H), 5.78 (s, 1H), 4.99 (s, 1H), |
| | 4.93-4.80 (m, 1H), 3.88 (s, 2H), 3.69-3.61 (m, 2H), 3.59-3.46 (m, 8H), 3.26-3.03 (m, |
| | 3H), 2.92-2.64 (m, 6H), 2.59-2.48 (m, 1H), 2.33-2.21 (m, 2H). |
| TDH-265 | 1H NMR (500 MHz, CDCl3) δ 9.06 (s, 0.5H), 9.01-9.00 (m, 0.5H), 8.54-8.48 (m, 1H), |
| | 8.09-8.01 (m, 1H), 7.99-7.94 (m, 1H), 7.75-7.67 (m, 2H), 7.67-7.61 (m, 3H), 7.58-7.48 |
| | (m, 3H), 7.45-7.34 (m, 3H), 7.18-7.10 (m, 1H), 7.03-6.93 (m, 2H), 6.50-6.35 (m, 3H), |
| | 5.03-4.91 (m, 1H), 4.91-4.83 (m, 0.5H), 4.77-4.68 (m, 0.5H), 4.68-4.55 (m, 2H), 3.71- |
| | 3.59 (m, 9H), 3.55-3.45 (m, 1H), 3.29-3.02 (m, 3H), 2.93-2.54 (m, 6H), 2.53-2.39 (m, |
| | 1H), 2.38-2.21 (m, 1H), 2.14-2.05 (m, 1H). |
| TDH-266 | 1H NMR (500 MHz, CDCl3) δ 9.63-9.40 (m, 1H), 9.02 (s, 1H), 8.50 (s, 1H), 8.08 (s, 1H), |
| | 7.96 (s, 1H), 7.79 (d, J = 8.7 Hz, 1H), 7.71-7.60 (m, 2H), 7.60-7.51 (m, 2H), 7.46-7.33 |
| | (m, 4H), 7.23-7.17 (m, 1H), 7.17-7.04 (m, 1H), 7.03-6.94 (m, 2H), 6.57 (s, 2H), 6.48- |
| | 6.40 (m, 1H), 5.03-4.82 (m, 2H), 4.61 (s, 2H), 3.71-3.60 (m, 2H), 3.59-3.51 (m, 8H), |
| | 3.15-2.94 (m, 3H), 2.91-2.60 (m, 6H), 2.55-2.44 (m, 1H), 2.23-2.14 (m, 1H), 2.14-2.07 |
| | (m, 1H). |
| TDH-267 | 1H NMR (500 MHz, CDCl3) δ 10.14 (s, 1H), 9.02 (s, 1H), 8.54-8.49 (m, 1H), 8.07 (d, J = |
| | 7.3 Hz, 1H), 7.95 (s, 1H), 7.69-7.60 (m, 2H), 7.59-7.48 (m, 3H), 7.45-7.36 (m, 3H), 7.18 |
| | (d, J = 7.2 Hz, 1H), 7.13-7.03 (m, 1H), 7.03-6.93 (m, 2H), 6.84-6.77 (m, 1H), 6.76-6.68 |
| | (m, 1H), 6.64 (s, 2H), 6.45 (d, J = 4.4 Hz, 1H), 5.04-4.92 (m, 1H), 4.92-4.79 (m, 1H), |
| | 4.01-3.90 (m, 2H), 3.66-3.56 (m, 2H), 3.53-3.37 (m, 8H), 3.18-2.93 (m, 3H), 2.90-2.61 |
| | (m, 6H), 2.55-2.44 (m, 1H), 2.25-2.13 (m, 1H), 2.14-2.06 (m, 1H). |
| TDH-268 | 1H NMR (300 MHz, CDCl3) δ 9.59 (s, 1H), 9.13-9.01 (m, 1H), 8.55-8.44 (m, 1H), 8.09 (s, |
| | 1H), 7.95 (s, 1H), 7.72-7.60 (m, 2H), 7.60-7.49 (m, 3H), 7.49-7.34 (m, 3H), 7.05-6.93 |
| | (m, 2H), 6.93-6.84 (m, 1H), 6.77-6.66 (m, 1H), 6.57 (s, 2H), 6.47-6.40 (m, 1H), 5.88- |
| | 5.69 (m, 1H), 5.03-4.91 (m, 1H), 4.91-4.77 (m, 1H), 3.93-3.80 (m, 2H), 3.70-3.59 (m, |
| | 2H), 3.58-3.40 (m, 8H), 3.25-3.01 (m, 3H), 2.89-2.76 (m, 3H), 2.76-2.59 (m, 3H), 2.57- |
| | 2.42 (m, 1H), 2.32-2.18 (m, 2H). |
| TDH-269 | 1H NMR (500 MHz, CDCl3) δ 9.08 (s, 0.5 H), 9.03 (d, J = 7.5 Hz, 0.5 H), 8.54-8.48 (m, |
| | 1H), 8.08-8.00 (m, 1H), 7.99-7.94 (m, 1H), 7.73-7.67 (m, 2H), 7.68-7.61 (m, 3H), 7.59- |
| | 7.47 (m, 3H), 7.45-7.34 (m, 3H), 7.18-7.09 (m, 1H), 7.03-6.94 (m, 2H), 6.52-6.36 (m, |
| | 3H), 5.02-4.91 (m, 1H), 4.91-4.84 (m, 0.5H), 4.78-4.69 (m, 0.5H), 4.66-4.55 (m, 2H), |
| | 3.68-3.58 (m, 9H), 3.55-3.45 (m, 1H), 3.27-3.01 (m, 3H), 2.90-2.56 (m, 6H), 2.52-2.40 |
| | (m, 1H), 2.37-2.21 (m, 1H), 2.12-2.02 (m, 1H). |
| TDH-270 | 1H NMR (500 MHz, CDCl3) δ 9.70-9.41 (m, 1H), 9.09-8.99 (m, 1H), 8.50 (d, J = 2.7 Hz, |
| | 1H), 8.08 (s, 1H), 7.95 (d, J = 2.7 Hz, 1H), 7.82-7.76 (m, 1H), 7.64 (d, J = 7.3 Hz, 2H), |
| | 7.59-7.52 (m, 2H), 7.46-7.34 (m, 4H), 7.23-7.17 (m, 1H), 7.17-7.06 (m, 1H), 7.02-6.95 |
| | (m, 2H), 6.57 (s, 2H), 6.47-6.41 (m, 1H), 5.00-4.84 (m, 2H), 4.65-4.57 (m, 2H), 3.68- |
| | 3.60 (m, 2H), 3.60-3.49 (m, 8H), 3.15-2.97 (m, 3H), 2.88-2.61 (m, 6H), 2.54-2.43 (m, |
| | 1H), 2.24-2.14 (m, 1H), 2.14-2.07 (m, 1H). |
| TDH-271 | 1H NMR (400 MHz, MeOH-d4) δ 8.62 (m, 1H), 8.27 (m, 1H), 8.03 (m, 1H), 7.74 (d, J = 7.5 |
| | Hz, 2H), 7.61 (m, 3H), 7.48 (m, 3H), 7.18 (d, J = 7.2 Hz, 1H), 7.08 (t, J = 8.8 Hz, 2H), 6.95 |
| | (d, J = 8.5 Hz, 1H), 6.35 (s, 1H), 5.10 (dd, J = 12.5, 5.4 Hz, 1H), 4.73 (t, J = 8.4 Hz, 1H), |
| | 4.60 (m, 2H), 4.52-4.42 (m, 1H), 4.24 (m, 3H), 3.95 (dd, J = 10.4, 4.9 Hz, 1H), 3.18 (d, J = |
| | 4.1 Hz, 2H), 3.10 (d, J = 11.5 Hz, 2H), 3.02 (d, J = 11.0 Hz, 2H), 2.91-2.81 (m, 1H), 2.77 |
| | (d, J = 12.8 Hz, 1H), 2.36 (d, J = 6.9 Hz, 2H), 2.27-2.12 (m, 6H), 2.05 (d, J = 9.6 Hz, 4H), |
| | 1.82 (m, 3H), 1.59 (m, 3H). |
| TDH-272 | 1H NMR (300 MHz, MeOH-d4) δ 8.61 (m, 1H), 8.27 (m, 1H), 8.02 (m, 1H), 7.77-7.69 (m, |
| | 2H), 7.68 (s, 1H), 7.60 (m, 1H), 7.47 (q, J = 7.5, 6.9 Hz, 3H), 7.39 (d, J = 2.3 Hz, 1H), 7.27 |
| | (d, J = 8.8 Hz, 1H), 7.07 (t, J = 8.8 Hz, 2H), 6.35 (s, 1H), 5.08 (dd, J = 12.2, 5.4 Hz, 1H), |
| | 4.27 (m, 1H), 4.01 (d, J = 14.0 Hz, 3H), 3.24-3.13 (m, 3H), 3.11 (m, 2H), 2.97 (d, J = 11.3 |
| | Hz, 2H), 2.85-2.76 (m, 2H), 2.34 (m, 7H), 2.17 (m, 4H), 2.05 (m, 6H), 1.83 (m, 3H), 1.60 |
| | (m, 3H). |
| TDH-273 | ¹H NMR (400 MHz, DMSO-d₆) δ 11.09 (s, 1H), 10.64 (s, 1H), 8.70 (s, 1H), 8.31 (s, 1H), |
| | 7.94 (s, 1H), 7.77-7.66 (m, 3H), 7.6-7.59 (m, 2H), 7.53-7.40 (m, 3H), 7.40-7.25 (m, 4H), |
| | 7.22-7.11 (m, 2H), 6.24 (s, 1H), 5.15-5.06 (m, 1H), 4.58-4.47 (m, 2H), 4.47-4.37 (m, |
| | 1H), 4.22-4.11 (m, 1H), 4.09-4.00 (m, 1H), 3.77-3.69 (m, 2H), 3.04-2.93 (m, 3H), 2.91- |
| | 2.82 (m, 2H), 2.77-2.56 (m, 4H), 2.20-1.99 (m, 4H), 1.95-1.64 (m, 9H), 1.65-1.47 (m, |
| | 1H), 1.47-1.31 (m, 1H). |
| TDH-274 | ¹H NMR (400 MHz, DMSO-d₆) δ 11.07 (s, 1H), 10.64 (s, 1H), 8.69 (s, 1H), 8.30 (s, 1H), |
| | 7.94 (s, 1H), 7.76-7.70 (m, 2H), 7.70-7.58 (m, 3H), 7.55-7.40 (m, 3H), 7.38-7.21 (m, |
| | 4H), 7.21-7.11 (m, 2H), 6.24 (s, 1H), 5.11-5.03 (m, 1H), 4.57-4.45 (m, 2H), 4.45-4.33 |
| | (m, 1H), 4.21-4.11 (m, 1H), 4.11-4.00 (m, 3H), 3.17-2.94 (m, 5H), 2.94-2.83 (m, 1H), |
| | 2.79-2.70 (m, 1H), 2.67-2.57 (m, 2H), 2.22-1.97 (m, 4H), 1.94-1.83 (m, 1H), 1.83-1.51 |
| | (m, 9H), 1.40-1.30 (m, 1H). |
| TDH-275 | ¹H NMR (400 MHz, DMSO-d₆) δ 11.10 (s, 1H), 10.64 (s, 1H), 8.70 (s, 1H), 8.31 (s, 1H), |
| | 7.95 (s, 1H), 7.77-7.70 (m, 2H), 7.68-7.58 (m, 3H), 7.55-7.41 (m, 3H), 7.35-7.27 (m, |
| | 2H), 7.22-7.05 (m, 5H), 6.24 (s, 1H), 5.14-5.04 (m, 1H), 4.59-4.46 (m, 2H), 4.46-4.36 |
| | (m, 1H), 4.31-4.14 (m, 3H), 3.98-3.88 (m, 1H), 3.20-3.11 (m, 1H), 3.07-2.99 (m, 1H), |
| | 2.95-2.73 (m, 4H), 2.65-2.57 (m, 2H), 2.18-2.02 (m, 3H), 1.98-1.85 (m, 1H), 1.78-1.59 |
| | (m, 4H), 1.53-1.40 (m, 1H). |
| TDH-276 | ¹H NMR (400 MHz, DMSO-d₆) δ 11.06 (s, 1H), 10.63 (s, 1H), 8.70 (s, 1H), 8.30 (s, 1H), |
| | 7.94 (s, 1H), 7.77-7.68 (m, 2H), 7.68-7.55 (m, 3H), 7.55-7.39 (m, 3H), 7.35-7.25 (m, |
| | 2H), 7.21-7.11 (m, 3H), 7.11-7.06 (m, 1H), 7.06-6.99 (m, 1H), 6.24 (s, 1H), 5.10-4.99 |
| | (m, 1H), 4.59-4.45 (m, 2H), 4.45-4.34 (m, 1H), 4.25-4.08 (m, 3H), 4.06-3.91 (m, 1H), |
| | 3.20-3.11 (m, 1H), 3.07-2.96 (m, 1H), 2.93-2.82 (m, 1H), 2.83-2.68 (m, 3H), 2.63-2.53 |
| | (m, 2H), 2.17-2.05 (m, 2H), 2.03-1.97 (m, 1H), 1.95-1.87 (m, 1H), 1.77-1.59 (m, 4H), |
| | 1.47-1.37 (m, 1H). |
| TDH-277 | ¹H NMR (400 MHz, DMSO-d₆) δ 11.11 (s, 1H), 10.64 (s, 1H), 8.70 (s, 1H), 8.31 (s, 1H), |
| | 7.94 (s, 1H), 7.82-7.76 (m, 1H), 7.76-7.70 (m, 2H), 7.67-7.58 (m, 2H), 7.52-7.41 (m, |
| | 4H), 7.38-7.26 (m, 3H), 7.23-7.12 (m, 2H), 6.24 (s, 1H), 5.31-5.16 (m, 2H), 5.16-5.07 |
| | (m, 1H), 4.60-4.46 (m, 2H), 4.37-4.26 (m, 1H), 4.22-4.11 (m, 1H), 3.87-3.78 (m, 1H), |
| | 3.19-3.15 (m, 1H), 3.08-2.95 (m, 1H), 2.95-2.84 (m, 1H), 2.80-2.71 (m, 2H), 2.65-2.55 |
| | (m, 2H), 2.14-1.99 (m, 3H), 1.95-1.86 (m, 1H), 1.77-1.59 (m, 4H), 1.50-1.36 (m, 1H). |
| TDH-278 | ¹H NMR (400 MHz, DMSO-d₆) δ 11.11 (s, 1H), 10.64 (s, 1H), 8.70 (s, 1H), 8.31 (s, 1H), |
| | 7.94 (s, 1H), 7.89-7.81 (m, 1H), 7.77-7.70 (m, 2H), 7.66-7.58 (m, 2H), 7.52-7.45 (m, |
| | 2H), 7.46-7.40 (m, 2H), 7.38-7.33 (m, 1H), 7.33-7.26 (m, 2H), 7.21-7.12 (m, 2H), 6.24 |
| | (s, 1H), 5.20-5.08 (m, 3H), 4.60-4.47 (m, 2H), 4.39-4.28 (m, 1H), 4.22-4.12 (m, 1H), |
| | 3.87-3.78 (m, 1H), 3.20-3.15 (m, 1H), 3.05-2.96 (m, 1H), 2.94-2.85 (m, 1H), 2.81-2.72 |
| | (m, 2H), 2.63-2.55 (m, 2H), 2.16-2.00 (m, 3H), 1.96-1.86 (m, 1H), 1.77-1.63 (m, 4H), |
| | 1.47-1.37 (m, 1H). |
| TDH-279 | ¹H NMR (400 MHz, DMSO-d₆) δ 11.09 (s, 1H), 10.63 (s, 1H), 8.70 (s, 1H), 8.32 (s, 1H), |
| | 7.94 (s, 1H), 7.76-7.67 (m, 3H), 7.64-7.58 (m, 2H), 7.53-7.47 (m, 2H), 7.46-7.40 (m, |
| | 1H), 7.38-7.28 (m, 4H), 7.21-7.11 (m, 2H), 6.24 (s, 1H), 5.15-5.06 (m, 1H), 4.59-4.49 |
| | (m, 2H), 4.23-4.10 (m, 1H), 3.77-3.71 (m, 2H), 3.07-2.86 (m, 5H), 2.80-2.73 (m, 1H), |
| | 2.66-2.58 (m, 2H), 2.16-1.99 (m, 4H), 1.84-1.74 (m, 5H). |
| TDH-280 | ¹H NMR (400 MHz, DMSO-d₆) δ 11.08 (s, 1H), 10.64 (s, 1H), 8.71 (s, 1H), 8.31 (s, 1H), |
| | 7.94 (s, 1H), 7.78-7.71 (m, 2H), 7.71-7.66 (m, 1H), 7.66-7.60 (m, 2H), 7.54-7.48 (m, |
| | 2H), 7.47-7.41 (m, 1H), 7.35-7.24 (m, 4H), 7.21-7.13 (m, 2H), 6.24 (s, 1H), 5.14-5.03 |
| | (m, 1H), 4.54-4.46 (m, 2H), 4.24-4.13 (m, 1H), 4.12-4.06 (m, 2H), 3.20-3.00 (m, 4H), |
| | 2.95-2.85 (m, 1H), 2.80-2.71 (m, 1H), 2.64-2.55 (m, 2H), 2.21-1.98 (m, 4H), 1.94-1.87 |
| | (m, 1H), 1.76-1.63 (m, 4H). |
| TDH-281 | 1H NMR (500 MHz, CDCl3) δ 9.14-8.98 (m, 1H), 8.60-8.49 (m, 1H), 8.23-8.21 (m, 0.2H), |
| | 8.15-8.13 (m, 0.2H), 8.10-8.07 (m, 0.3H), 7.98-7.93 (m, 1H), 7.66 (d, J = 7.7 Hz, 1H), |
| | 7.64-7.57 (m, 1H), 7.56-7.48 (m, 2H), 7.45-7.38 (m, 2H), 7.37-7.32 (m, 1H), 7.16-7.12 |
| | (m, 1H), 7.11-7.04 (m, 1H), 7.02-6.95 (m, 2H), 6.88-6.77 (m, 1H), 6.60-6.34 (m, 3H), |
| | 5.01-4.96 (m, 1H), 4.96-4.90 (m, 1H), 4.65-4.52 (m, 1H), 4.06-3.88 (m, 1H), 3.88-3.70 |
| | (m, 1H), 3.70-3.42 (m, 1H), 3.14-2.92 (m, 2H), 2.92-2.64 (m, 5H), 2.62-2.38 (m, 3H), |
| | 2.36-2.19 (m, 2H), 2.19-2.07 (m, 2H), 1.96-1.87 (m, 1H), 1.81-1.69 (m, 2H), 1.21-1.01 |
| | (m, 2H). |
| TDH-282 | 1H NMR (500 MHz, CDCl3) δ 9.17-9.07 (m, 0.5H), 9.07-8.95 (m, 0.5H), 8.58-8.50 (m, |
| | 1H), 8.48-8.38 (m, 1H), 8.23 (s, 0.3H), 8.16 (s, 0.3H), 8.08 (s, 0.4H), 7.98-7.91 (m, 1H), |
| | 7.72-7.61 (m, 3H), 7.60-7.50 (m, 2H), 7.46-7.36 (m, 2H), 7.34-7.28 (m, 1H), 7.02-6.96 |
| | (m, 2H), 6.96-6.89 (m, 1H), 6.84-6.77 (m, 1H), 6.49-6.45 (m, 1H), 6.40 (s, 2H), 5.90- |
| | 5.84 (m, 0.7H), 5.82-5.78 (m, 0.3H), 5.03-4.91 (m, 2H), 4.64-4.52 (m, 1H), 3.92-3.76 (m, |
| | 1H), 3.74-3.59 (m, 1H), 3.57-3.35 (m, 1H), 3.10-2.96 (m, 2H), 2.93-2.69 (m, 5H), 2.66- |
| | 2.45 (m, 3H), 2.40-2.23 (m, 2H), 2.20-2.07 (m, 2H), 2.00-1.90 (m, 1H), 1.85-1.72 (m, |
| | 2H), 1.18-1.05 (m, 2H). |
| TDH-283 | 1H NMR (400 MHz, CDCl3) δ 9.03-8.90 (m, 1H), 8.60-8.53 (m, 0.5H), 8.51-8.50 (m, |
| | 0.5H), 8.10-8.02 (m, 1H), 8.02-7.92 (m, 1H), 7.74-7.61 (m, 3H), 7.60-7.47 (m, 3H), |
| | 7.46-7.29 (m, 4H), 7.06-6.90 (m, 3H), 6.72 (s, 1H), 6.47-6.40 (m, 1H), 6.35 (s, 1H), |
| | 5.08-4.87 (m, 3H), 4.87-4.74 (m, 1H), 4.62-4.49 (m, 1H), 4.40-4.37 (m, 0.4H), 4.23-4.12 |
| | (m, 0.6H), 3.23-3.02 (m, 2H), 3.01-2.56 (m, 6H), 2.55-2.41 (m, 2H), 2.39-2.23 (m, 1H), |
| | 2.22-2.06 (m, 3H), 2.02-1.95 (m, 1H), 1.94-1.77 (m, 2H), 1.15-0.92 (m, 2H). |
| TDH-284 | 1H NMR (500 MHz, CDCl3) δ 9.13-9.04 (m, 1H), 9.04-8.93 (m, 1H), 8.55-8.49 (m, 1H), |
| | 8.15 (s. 0.4H), 8.09-8.04 (m, 0.6H), 7.98-7.93 (m, 1H), 7.81-7.75 (m, 1H), 7.68-7.60 (m, |
| | 2H), 7.58-7.50 (m, 2H), 7.44-7.36 (m, 3H), 7.35-7.29 (m, 1H), 7.29-7.24 (m, 1H), 7.02- |
| | 6.95 (m, 2H), 6.54-6.37 (m, 3H), 5.02-4.92 (m, 2H), 4.89-4.73 (m, 1H), 4.70-4.47 (m, |
| | 2H), 3.79-3.60 (m, 1H), 3.11-2.97 (m, 2H), 2.93-2.69 (m, 5H), 2.65-2.46 (m, 3H), 2.43- |
| | 2.27 (m, 2H), 2.22-2.09 (m, 2H), 1.82-1.75 (m, 1H), 1.75-1.60 (m, 2H), 1.18-1.04 (m, 2H). |
| TDH-285 | 1H NMR (500 MHz, CDCl3) δ 9.19-9.16 (m, 0.3H), 9.13-9.12 (m, 0.4H), 9.05 (s, 0.2H), |
| | 8.59-8.52 (m, 1H), 8.23 (d, J = 9.0 Hz, 0.3 H), 8.16 (d, J = 7.0 Hz, 0.3 H), 8.11-8.10 (m, |
| | 0.4H), 8.00-7.94 (m, 1H), 7.71-7.63 (m, 2H), 7.62-7.49 (m, 3H), 7.47-7.40 (m, 2H), 7.40- |
| | 7.35 (m, 1H), 7.19-7.07 (m, 2H), 7.04-6.97 (m, 2H), 6.89-6.77 (m, 1H), 6.64-6.45 (m, |
| | 3H), 5.04-4.98 (m, 1H), 4.97-4.92 (m, 1H), 4.64-4.54 (m, 1H), 4.07-3.89 (m, 1H), 3.88- |
| | 3.69 (m, 1H), 3.68-3.48 (m, 1H), 3.14-2.96 (m, 2H), 2.94-2.68 (m, 5H), 2.68-2.45 (m, |
| | 3H), 2.41-2.23 (m, 2H), 2.21-2.09 (m, 2H), 2.07-1.96 (m, 1H), 1.96-1.85 (m, 2H), 1.21- |
| | 1.04 (m, 2H). |
| TDH-286 | 1H NMR (500 MHz, CDCl3) δ 9.17-8.97 (m, 1H), 8.60-8.48 (m, 2H), 8.21 (s, 0.3H), 8.15 |
| | (s, 0.3H), 8.08 (s, 0.4H), 7.98-7.91 (m, 1H), 7.73-7.49 (m, 5H), 7.46-7.36 (m, 2H), 7.36- |
| | 7.29 (m, 1H), 7.03-6.96 (m, 2H), 6.96-6.89 (m, 1H), 6.86-6.76 (m, 1H), 6.54-6.30 (m, |
| | 3H), 5.96-5.77 (m, 1H), 5.03-4.90 (m, 2H), 4.66-4.52 (m, 1H), 3.97-3.78 (m, 1H), 3.78- |
| | 3.61 (m, 1H), 3.61-3.39 (m, 1H), 3.10-2.95 (m, 2H), 2.93-2.66 (m, 5H), 2.66-2.41 (m, |
| | 3H), 2.40-2.22 (m, 2H), 2.23-2.09 (m, 2H), 2.01-1.92 (m, 1H), 1.88-1.75 (m, 2H), 1.21- |
| | 1.02 (m, 2H). |
| TDH-287 | 1H NMR (500 MHz, CDCl3) δ 9.07-8.95 (m, 1H), 8.60-8.47 (m, 1H), 8.11-8.03 (m, 1H), |
| | 8.03-7.93 (m, 1H), 7.75-7.61 (m, 3H), 7.60-7.46 (m, 3H), 7.46-7.37 (m, 3H), 7.37-7.29 |
| | (m, 1H), 7.06-6.88 (m, 3H), 6.72 (s, 1H), 6.49-6.41 (m, 1H), 5.09-4.91 (m, 3H), 4.91- |
| | 4.74 (m, 1H), 4.65-4.48 (m, 1H), 4.41-4.38 (m, 0.4H), 4.25-4.13 (m, 0.7H), 3.24-3.02 (m, |
| | 2H), 3.02-2.65 (m, 5H), 2.65-2.43 (m, 3H), 2.40-2.01 (m, 5H), 2.01-1.83 (m, 2H), 1.15- |
| | 0.94 (m, 2H). |
| TDH-288 | 1H NMR (500 MHz, CDCl3) δ 9.13-9.02 (m, 1H), 9.02-8.85 (m, 1H), 8.56-8.48 (m, 1H), |
| | 8.14 (s, 1H), 8.06 (s, 1H), 7.98-7.92 (m, 1H), 7.82-7.74 (m, 1H), 7.68-7.60 (m, 2H), |
| | 7.59-7.49 (m, 2H), 7.44-7.35 (m, 3H), 7.35-7.29 (m, 1H), 7.03-6.94 (m, 2H), 6.53-6.38 |
| | (m, 3H), 5.02-4.91 (m, 2H), 4.83-4.71 (m, 1H), 4.67-4.46 (m, 2H), 3.79-3.61 (m, 1H), |
| | 3.09-2.97 (m, 2H), 2.94-2.68 (m, 5H), 2.65-2.43 (m, 3H), 2.38-2.20 (m, 2H), 2.19-2.08 |
| | (m, 2H), 2.05-1.96 (m, 1H), 1.94-1.82 (m, 2H), 1.18-1.04 (m, 2H). |
| TDH-289 | ¹H NMR (400 MHz, DMSO-d₆) δ 11.14 (s, 1H), 10.63 (s, 1H), 8.69 (s, 1H), 8.28 (s, 1H), |
| | 7.95-7.89 (m, 2H), 7.76-7.70 (m, 2H), 7.66-7.58 (m, 3H), 7.53-7.46 (m, 2H), 7.46-7.40 |
| | (m, 1H), 7.39-7.33 (m, 1H), 7.33-7.24 (m, 2H), 7.19-7.11 (m, 2H), 6.24 (s, 1H), 5.97- |
| | 5.83 (m, 1H), 4.44-4.37 (m, 1H), 4.26-4.14 (m, 1H), 4.05-3.93 (m, 1H), 3.06-2.93 (m, |
| | 4H), 2.91-2.75 (m, 4H), 2.71-2.63 (m, 2H), 2.44-2.32 (m, 2H), 2.26-2.18 (m, 1H), 2.08- |
| | 1.87 (m, 8H), 1.81-1.62 (m, 5H), 1.62-1.49 (m, 2H), 1.46-1.35 (m, 2H), 1.12-1.03 (m, |
| | 1H), 1.03-0.90 (m, 1H). |
| TDH-290 | ¹H NMR (400 MHz, DMSO-d₆) δ 11.14 (s, 1H), 10.64 (s, 1H), 8.70 (s, 1H), 8.29 (s, 1H), |
| | 8.07-7.97 (m, 1H), 7.97-7.89 (m, 1H), 7.78-7.69 (m, 3H), 7.68-7.58 (m, 2H), 7.52-7.37 |
| | (m, 4H), 7.34-7.23 (m, 2H), 7.21-7.12 (m, 2H), 6.24 (s, 1H), 5.95-5.83 (m, 1H), 4.42- |
| | 4.34 (m, 1H), 4.24-4.15 (m, 1H), 4.13-4.06 (m, 2H), 4.06-3.96 (m, 1H), 3.17-3.09 (m, |
| | 2H), 3.01-2.97 (m, 3H), 2.77-2.61 (m, 3H), 2.41-2.31 (m, 2H), 2.27-2.20 (m, 1H), 2.15- |
| | 1.90 (m, 8H), 1.77-1.58 (m, 7H), 1.46-1.40 (m, 2H), 1.13-1.03 (m, 1H), 1.03-0.93 (m, 1H). |
| TDH-291 | ¹H NMR (400 MHz, DMSO-d₆) δ 11.14 (s, 1H), 10.64 (s, 1H), 8.70 (s, 1H), 8.29 (s, 1H), |
| | 8.04-7.98 (m, 1H), 7.92 (s, 1H), 7.79-7.71 (m, 2H), 7.68-7.57 (m, 2H), 7.54-7.40 (m, |
| | 3H), 7.35-7.25 (m, 2H), 7.19-7.11 (m, 3H), 6.95-6.87 (m, 1H), 6.24 (s, 1H), 5.94-5.85 |
| | (m, 1H), 4.43-4.35 (m, 1H), 4.32-4.24 (m, 2H), 4.20-4.16 (m, 2H), 3.97-3.89 (m, 1H), |
| | 3.67-3.58 (m, 1H), 3.04-2.92 (m, 4H), 2.72-2.59 (m, 3H), 2.43-2.32 (m, 2H), 2.28-2.20 |
| | (m, 1H), 2.14-1.90 (m, 7H), 1.77-1.69 (m, 2H), 1.62-1.51 (m, 1H), 1.46-1.37 (m, 2H), |
| | 1.18-1.09 (m, 1H), 1.07-0.96 (m, 1H). |
| TDH-292 | ¹H NMR (400 MHz, DMSO-d₆) δ 11.13 (s, 1H), 10.64 (s, 1H), 8.70 (s, 1H), 8.29 (s, 1H), |
| | 7.95-7.89 (m, 2H), 7.77-7.70 (m, 2H), 7.67-7.58 (m, 2H), 7.55-7.41 (m, 4H), 7.36-7.20 |
| | (m, 3H), 7.20-7.08 (m, 3H), 6.24 (s, 1H), 5.91-5.82 (m, 1H), 4.42-4.34 (m, 1H), 4.26- |
| | 4.08 (m, 3H), 4.00-3.91 (m, 1H), 3.11-2.92 (m, 4H), 2.73-2.59 (m, 3H), 2.45-2.35 (m, |
| | 2H), 2.26-2.18 (m, 1H), 2.10-1.90 (m, 6H), 1.80-1.68 (m, 2H), 1.66-1.53 (m, 1H), 1.46- |
| | 1.37 (m, 2H), 1.22-1.16 (m, 1H), 1.08-0.95 (m, 1H). |
| TDH-293 | ¹H NMR (400 MHz, DMSO-d₆) δ 11.19 (s, 1H), 10.64 (s, 1H), 8.70 (s, 1H), 8.29 (s, 1H), |
| | 8.24-8.18 (m, 1H), 7.92 (s, 1H), 7.77-7.67 (m, 3H), 7.67-7.58 (m, 2H), 7.58-7.54 (m, |
| | 1H), 7.54-7.47 (m, 2H), 7.47-7.40 (m, 1H), 7.35-7.25 (m, 2H), 7.21-7.12 (m, 2H), 6.24 |
| | (s, 1H), 5.99-5.92 (m, 1H), 5.25-5.12 (m, 2H), 4.35-4.25 (m, 1H), 3.88-3.80 (m, 1H), |
| | 3.07-2.94 (m, 4H), 2.74-2.61 (m, 3H), 2.39-2.33 (m, 2H), 2.33-2.23 (m, 1H), 2.08-1.95 |
| | (m, 7H), 1.78-1.70 (m, 2H), 1.65-1.57 (m, 1H), 1.46-1.41 (m, 2H), 1.05-0.95 (m, 1H), |
| | 0.92-0.80 (m, 1H). |
| TDH-294 | ¹H NMR (400 MHz, DMSO-d₆) δ 11.15 (s, 1H), 10.63 (s, 1H), 8.70 (s, 1H), 8.30 (s, 1H), |
| | 7.96-7.89 (m, 2H), 7.77-7.70 (m, 2H), 7.65-7.58 (m, 3H), 7.53-7.41 (m, 3H), 7.39-7.35 |
| | (m, 1H), 7.32-7.25 (m, 2H), 7.20-7.11 (m, 2H), 6.24 (s, 1H), 5.97-5.87 (m, 1H), 4.26- |
| | 4.14 (m, 1H), 3.42-3.39 (m, 2H), 3.07-2.90 (m, 4H), 2.78-2.68 (m, 4H), 2.44-2.35 (m, |
| | 2H), 2.28-2.23 (m, 1H), 2.08-1.95 (m, 6H), 1.81-1.74 (m, 2H), 1.49-1.45 (m, 4H). |
| TDH-295 | ¹H NMR (400 MHz, DMSO-d6) δ 11.14 (s, 1H), 10.62 (s, 1H), 8.70 (s, 1H), 8.29 (s, 1H), |
| | 8.03-7.97 (m, 1H), 7.92 (s, 1H), 7.76-7.68 (m, 3H), 7.67-7.58 (m, 2H), 7.54-7.47 (m, |
| | 2H), 7.47-7.40 (m, 1H), 7.39-7.35 (m, 1H), 7.32-7.24 (m, 2H), 7.21-7.13 (m, 2H), 6.24 |
| | (s, 1H), 5.93-5.84 (m, 1H), 4.22-4.14 (m, 1H), 4.12-4.06 (m, 2H), 3.06-2.90 (m, 6H), |
| | 2.75-2.61 (m, 3H), 2.43-2.35 (m, 2H), 2.26-2.19 (m, 1H), 2.07-1.99 (m, 4H), 1.84-1.75 |
| | (m, 2H), 1.68-1.61 (m, 1H), 1.48-1.40 (m, 2H), 1.27-1.20 (m, 2H). |
| TDH-296 | 1H NMR (500 MHz, CDCl3) δ 9.02-8.91 (m, 1H), 8.55 (d, J = 7.5 Hz, 1H), 8.17-8.08 (m, |
| | 1H), 8.02 (s, 1H), 7.64 (d, J = 7.2 Hz, 2H), 7.59-7.47 (m, 3H), 7.47-7.35 (m, 3H), 7.21- |
| | 7.11 (m, 2H), 7.00 (t, J = 8.5 Hz, 2H), 6.86-6.77 (m, 1H), 6.54-6.33 (m, 3H), 5.04-4.88 |
| | (m, 2H), 4.66-4.56 (m, 1H), 4.08-3.91 (m, 2H), 3.86-3.68 (m, 3H), 3.61-3.47 (m, 2H), |
| | 3.03-2.59 (m, 5H), 2.55-2.39 (m, 2H), 2.16-1.97 (m, 2H), 1.94-1.73 (m, 2H), 1.19-1.04 |
| | (m, 2H). |
| TDH-297 | 1H NMR (400 MHz, CDCl3) δ 8.99-8.90 (m, 1H), 8.53 (s, 1H), 8.43-8.34 (m, 1H), 8.09 (s, |
| | 1H), 8.02 (s, 1H), 7.69-7.60 (m, 3H), 7.59-7.49 (m, 2H), 7.48-7.35 (m, 3H), 7.04-6.92 |
| | (m, 3H), 6.82 (d, J = 8.3 Hz, 1H), 6.45 (s, 1H), 6.38 (s, 2H), 5.94-5.85 (m, 1H), 5.04-4.89 |
| | (m, 2H), 4.68-4.56 (m, 1H), 3.97-3.89 (m, 1H), 3.89-3.77 (m, 3H), 3.74-3.63 (m, 1H), |
| | 3.61-3.47 (m, 2H), 3.08-2.94 (m, 1H), 2.94-2.63 (m, 4H), 2.56-2.40 (m, 2H), 2.19-2.09 |
| | (m, 1H), 2.08-1.99 (m, 1H), 1.96-1.78 (m, 2H), 1.21-1.09 (m, 2H). |
| TDH-298 | 1H NMR (500 MHz, DMSO-d6) δ 11.12 (s, 1H), 10.72 (s, 1H), 8.72 (s, 1H), 8.38 (s, 1H), |
| | 7.99 (s, 1H), 7.80-7.70 (m, 3H), 7.65-7.58 (m, 2H), 7.49 (t, J = 7.5 Hz, 2H), 7.46-7.42 (m, |
| | 2H), 7.35-7.28 (m, 3H), 7.15 (t, J = 8.8 Hz, 2H), 6.25 (s, 1H), 5.23-5.13 (m, 2H), 5.14- |
| | 5.08 (m, 1H), 5.07-5.00 (m, 1H), 4.31-4.24 (m, 1H), 3.82-3.75 (m, 1H), 3.75-3.70 (m, |
| | 2H), 3.17 (d, J = 5.2 Hz, 2H), 3.06-3.00 (m, 1H), 2.93-2.84 (m, 1H), 2.65-2.56 (m, 3H), |
| | 2.42 (d, J = 7.0 Hz, 2H), 2.06-2.01 (m, 1H), 1.77-1.70 (m, 2H), 1.04-0.95 (m, 2H). |
| TDH-299 | 1H NMR (500 MHz, CDCl3) δ 9.15 (s, 1H), 9.07 (s, 1H), 8.97-8.91 (m, 1H), 8.53 (s, 1H), |
| | 8.06 (d, J = 4.8 Hz, 1H), 8.01 (s, 1H), 7.80 (d, J = 8.2 Hz, 1H), 7.66-7.61 (m, 2H), 7.56- |
| | 7.51 (m, 2H), 7.44-7.38 (m, 3H), 7.36-7.32 (m, 1H), 7.32-7.27 (m, 1H), 6.99 (t, J = 8.5 |
| | Hz, 2H), 6.56-6.40 (m, 3H), 5.04-4.98 (m, 1H), 4.97-4.91 (m, 1H), 4.87-4.73 (m, 2H), |
| | 4.59-4.49 (m, 1H), 3.90-3.74 (m, 3H), 3.60-3.47 (m, 2H), 3.08-2.98 (m, 1H), 2.94-2.68 |
| | (m, 3H), 2.67-2.57 (m, 1H), 2.54-2.43 (m, 2H), 2.17-2.10 (m, 1H), 1.91-1.83 (m, 2H), |
| | 1.18-1.06 (m, 2H). |
| TDH-300 | 1H NMR (500 MHz, Chloroform-d) δ 10.78-10.15 (m, 1H), 9.29 (d, J = 3.2 Hz, 1H), 8.45- |
| | 8.32 (m, 2H), 7.80 (d, J = 8.3 Hz, 1H), 7.72-7.64 (m, 4H), 7.60-7.54 (m, 2H), 7.46-7.39 (m, |
| | 3H), 7.37 (t, J = 3.1 Hz, 1H), 7.29 (d, J = 2.3 Hz, 1H), 6.98 (t, J = 8.7 Hz, 2H), 6.72-6.53 |
| | (m, 2H), 6.38 (s, 1H), 4.99-4.92 (m, 1H), 4.89-4.83 (m, 2H), 4.52 (s, 1H), 4.20 (s, 1H), |
| | 3.91-3.82 (m, 1H), 3.07 (s, 2H), 2.93-2.70 (m, 4H), 2.68-2.56 (m, 2H), 2.47 (s, 2H), 2.16- |
| | 2.09 (m, 4H), 1.86-1.71 (m, 3H), 1.69-1.57 (m, 2H), 1.50 (s, 2H), 1.20-1.15 (m, 2H |
| TDH-301 | 1H NMR (500 MHz, Chloroform-d) δ 8.47-8.37 (m, 1H), 8.33-8.25 (m, 1H), 8.15-7.95 (m, |
| | 1H), 7.72-7.58 (m, 5H), 7.54-7.40 (m, 6H), 7.36 (d, J = 8.5 Hz, 1H), 7.02-6.95 (m, 2H), |
| | 6.65-6.47 (m, 2H), 6.41-6.34 (m, 1H), 5.02-4.79 (m, 3H), 4.57-4.44 (m, 1H), 4.25-4.17 (m, |
| | 1H), 3.77 (s, 1H), 3.18-2.98 (m, 4H), 2.96 (s, 1H), 2.88 (s, 2H), 2.78-2.68 (m, 1H), 2.67- |
| | 2.56 (m, 2H), 2.43 (s, 1H), 2.14-2.04 (m, 4H), 1.78-1.71 (m, 4H), 1.56-1.40 (m, 4H), 1.18- |
| | 1.05 (m, 2H). |
| TDH-302 | 1H NMR (500 MHz, Chloroform-d) δ 8.45-8.39 (m, 1H), 8.30 (d, J = 2.1 Hz, 1H), 7.80-7.70 |
| | (m, 2H), 7.70-7.25 (m, 3H), 7.53-7.44 (m, 4H), 7.10-7.01 (m, 2H), 6.97 (d, J = 2.2 Hz, 1H), |
| | 6.90-6.82 (m, 1H), 6.59-6.42 (m, 2H), 6.40 (s, 1H), 5.93 (s, 1H), 4.96 (dd, J = 12.2, 5.4 Hz, |
| | 1H), 4.85-4.61 (m, 1H), 4.20 (s, 1H), 3.97 (d, J = 3.8 Hz, 2H), 3.81-3.74 (m, 1H), 3.18-3.05 |
| | (m, 3H), 2.95-2.69 (m, 4H), 2.47 (s, 2H), 2.19-2.03 (m, 5H), 1.98-1.76 (m, 3H), 1.59-1.46 |
| | (m, 4H), 1.23 (s, 2H). |
| TDH-303 | 1H NMR (500 MHz, Chloroform-d) δ 8.44 (t, J = 3.0 Hz, 1H), 8.30 (d, J = 2.7 Hz, 1H), 7.79 |
| | (s, 1H), 7.73-7.61 (m, 4H), 7.53-7.45 (m, 5H), 7.24-7.14 (m, 2H), 7.03 (t, J = 8.6 Hz, 2H), |
| | 6.86-6.79 (m, 1H), 6.72-6.34 (m, 2H), 6.40 (s, 1H), 4.98-4.90 (m, 1H), 4.65 (s, 1H), 4.20 |
| | (s, 1H), 4.05 (d, J = 4.3 Hz, 2H), 3.84-3.71 (m, 2H), 3.14-3.04 (m, 3H), 2.93-2.70 (m, 4H), |
| | 2.46 (s, 2H), 2.17-2.05 (, 5H), 1.91-1.76 (m, 3H), 1.57-1.45(m, 4H), 1.25-1.15 (m, 2H). |
| TDH-304 | ¹H NMR (400 MHz, DMSO-d₆) δ 11.08 (s, 1H), 10.62 (s, 1H), 8.69 (s, 1H), 8.34-8.26 (m, |
| | 1H), 7.91 (s, 1H), 7.76-7.70 (m, 2H), 7.70-7.64 (m, 1H), 7.64-7.56 (m, 2H), 7.54-7.39 |
| | (m, 3H), 7.39-7.23 (m, 4H), 7.22-7.10 (m, 2H), 6.23 (s, 1H), 5.16-5.05 (m, 1H), 4.27- |
| | 4.13 (m, 1H), 4.00-3.81 (m, 2H), 3.80-3.66 (m, 2H), 3.60-3.40 (m, 2H), 3.28-3.11 (m, |
| | 2H), 3.12-2.93 (m, 5H), 2.93-2.77 (m, 3H), 2.64-2.54 (m, 2H), 2.26-2.15 (m, 2H), 2.05- |
| | 1.89 (m, 6H), 1.84-1.72 (m, 4H). |
| TDH-305 | ¹H NMR (400 MHz, DMSO-d₆) δ 11.08 (s, 1H), 10.62 (s, 1H), 8.69 (s, 1H), 8.36-8.24 (m, |
| | 1H), 7.91 (s, 1H), 7.76-7.69 (m, 2H), 7.69-7.58 (m, 3H), 7.53-7.46 (m, 2H), 7.46-7.39 |
| | (m, 1H), 7.36-7.31 (m, 1H), 7.31-7.20 (m, 3H), 7.19-7.11 (m, 2H), 6.23 (s, 1H), 5.14- |
| | 5.03 (m, 1H), 4.23-4.15 (m, 1H), 4.11-4.02 (m, 2H), 4.02-3.80 (m, 3H), 3.61-3.50 (m, |
| | 1H), 3.50-3.40 (m, 2H), 3.11-2.81 (m, 7H), 2.77-2.67 (m, 1H), 2.65-2.55 (m, 2H), 2.45- |
| | 2.38 (m, 2H), 2.10-1.89 (m, 6H), 1.79-1.58 (m, 4H). |
| TDH-306 | ¹H NMR (400 MHz, DMSO-d₆) δ 11.11 (s, 1H), 10.62 (s, 1H), 8.70 (s, 1H), 8.35-8.27 (m, |
| | 1H), 7.96-7.87 (m, 1H), 7.77-7.67 (m, 2H), 7.68-7.57 (m, 3H), 7.53-7.39 (m, 3H), 7.35- |
| | 7.24 (m, 2H), 7.22-7.01 (m, 5H), 6.24 (s, 1H), 5.13-5.03 (m, 1H), 4.42-4.26 (m, 2H), |
| | 4.22-4.13 (m, 2H), 3.91-3.87 (m, 1H), 3.85-3.76 (m, 1H), 3.60-3.47 (m, 2H), 3.47-3.36 |
| | (m, 1H), 3.23-3.12 (m, 1H), 3.05-2.91 (m, 3H), 2.89-2.82 (m, 1H), 2.62-2.57 (m, 2H), |
| | 2.26-2.16 (m, 2H), 2.06-1.94 (m, 6H). |
| TDH-307 | ¹H NMR (400 MHz, DMSO-d₆) δ 11.06 (s, 1H), 10.62 (s, 1H), 8.69 (s, 1H), 8.36-8.27 (m, |
| | 1H), 7.91 (s, 1H), 7.78-7.70 (m, 2H), 7.69-7.55 (m, 3H), 7.51-7.40 (m, 3H), 7.33-7.26 |
| | (m, 2H), 7.21-7.09 (m, 4H), 7.06-6.99 (m, 1H), 6.24 (s, 1H), 5.11-5.00 (m, 1H), 4.37- |
| | 4.00 (m, 4H), 3.89-3.81 (m, 2H), 3.57-3.50 (m, 1H), 3.26-3.10 (m, 2H), 3.06-2.94 (m, |
| | 3H), 2.92-2.77 (m, 2H), 2.62-2.55 (m, 2H), 2.25-2.16 (m, 2H), 2.04-1.93 (m, 6H). |
| TDH-308 | ¹H NMR (400 MHz, DMSO-d₆) δ 11.11 (s, 1H), 10.67 (s, 1H), 8.70 (s, 1H), 8.35-8.21 (m, |
| | 1H), 7.93 (s, 1H), 7.82-7.76 (m, 1H), 7.75-7.70 (m, 2H), 7.71-7.59 (m, 2H), 7.52-7.41 |
| | (m, 4H), 7.40-7.35 (m, 1H), 7.34-7.26 (m, 2H), 7.20-7.11 (m, 2H), 6.25 (s, 1H), 5.37- |
| | 5.21 (m, 1H), 5.21-5.08 (m, 2H), 4.29-4.16 (m, 1H), 4.16-4.06 (m, 1H), 3.99-3.82 (m, |
| | 2H), 3.82-3.67 (m, 2H), 3.27-3.16 (m, 2H), 3.06-2.75 (m, 4H), 2.63-2.56 (m, 2H), 2.28- |
| | 2.12 (m, 2H), 2.15-1.78 (m, 6H). |
| TDH-309 | ¹H NMR (400 MHz, DMSO-d₆) δ 11.11 (s, 1H), 10.65 (s, 1H), 8.69 (s, 1H), 8.28 (s, 1H), |
| | 7.93 (s, 1H), 7.88-7.81 (m, 1H), 7.80-7.67 (m, 2H), 7.67-7.56 (m, 2H), 7.54-7.41 (m, |
| | 4H), 7.41-7.35 (m, 1H), 7.35-7.25 (m, 2H), 7.21-7.09 (m, 2H), 6.24 (s, 1H), 5.31-5.19 |
| | (m, 1H), 5.19-5.02 (m, 2H), 4.33-4.09 (m, 2H), 3.99-3.81 (m, 2H), 3.81-3.65 (m, 2H), |
| | 3.28-3.15 (m, 2H), 3.06-2.76 (m, 4H), 2.64-2.57 (m, 2H), 2.30-2.16 (m, 2H), 2.16-1.84 |
| | (m, 6H). |
| TDH-310 | ¹H NMR (400 MHz, DMSO-d₆) δ 11.13-11.04 (m, 1H), 10.66 (s, 1H), 8.70 (s, 1H), 8.35- |
| | 8.23 (m, 1H), 7.94 (s, 1H), 7.82-7.70 (m, 3H), 7.67-7.58 (m, 2H), 7.53-7.46 (m, 2H), |
| | 7.46-7.35 (m, 3H), 7.35-7.26 (m, 2H), 7.21-7.11 (m, 2H), 6.25 (s, 1H), 5.17-5.04 (m, |
| | 1H), 4.33-4.09 (m, 1H), 4.04-3.93 (m, 1H), 3.91-3.68 (m, 3H), 3.27-3.12 (m, 2H), 3.06- |
| | 2.92 (m, 2H), 2.92-2.79 (m, 2H), 2.79-2.65 (m, 1H), 2.62-2.55 (m, 2H), 2.30-2.14 (m, |
| | 2H), 2.15-1.90 (m, 6H). |
| TDH-311 | ¹H NMR (400 MHz, DMSO-d₆) δ 11.09 (s, 1H), 10.65 (s, 1H), 8.70 (s, 1H), 8.31 (s, 1H), |
| | 7.94 (s, 1H), 7.83-7.68 (m, 3H), 7.68-7.56 (m, 2H), 7.54-7.46 (m, 2H), 7.46-7.35 (m, |
| | 2H), 7.35-7.24 (m, 3H), 7.22-7.06 (m, 2H), 6.25 (s, 1H), 5.15-5.05 (m, 1H), 4.30-4.16 |
| | (m, 1H), 4.03-3.93 (m, 2H), 3.91-3.79 (m, 2H), 3.76-3.56 (m, 2H), 3.27-3.15 (m, 2H), |
| | 2.98-2.86 (m, 2H), 2.76-2.68 (m, 1H), 2.63-2.54 (m, 2H), 2.28-2.16 (m, 2H), 2.13-1.91 |
| | (m, 6H). |
| TDH-312 | ¹H NMR (400 MHz, DMSO-d₆) δ 10.71 (s, 1H), 8.99 (s, 1H), 8.70 (s, 1H), 8.64-8.57 (m, |
| | 1H), 8.28 (s, 1H), 7.94 (s, 1H), 7.76-7.71 (m, 2H), 7.66-7.59 (m, 2H), 7.51-7.46 (m, 2H), |
| | 7.46-7.38 (m, 5H), 7.33-7.27 (m, 2H), 7.20-7.12 (m, 2H), 6.26 (s, 1H), 5.19-5.11 (m, |
| | 1H), 4.56-4.48 (m, 1H), 4.46-4.41 (m, 1H), 4.41-4.33 (m, 2H), 4.33-4.23 (m, 2H), 3.72- |
| | 3.57 (m, 4H), 3.04-2.96 (m, 2H), 2.91-2.77 (m, 4H), 2.45 (s, 3H), 2.13-1.99 (m, 8H), |
| | 1.95-1.86 (m, 2H), 1.75-1.63 (m, 3H), 1.27-1.12 (m, 4H), 0.95 (s, 9H). |
| TDH-313 | 1H NMR (400 MHz, DMSO-d6) δ 10.58 (s, 1H), 8.47 (s, 1H), 7.69-7.54 (m, 5H), 7.54- |
| | 7.40 (m, 3H), 7.40-7.33 (m, 1H), 7.20-7.12 (m, 2H), 6.19 (s, 1H). LC/MS (ESI) m/z 445.3 [M + H]+ |
| TDH-314 | 1H NMR (400 MHz, DMSO-d6) δ 10.86 (s, 1H), 9.13-8.92 (m, 1H), 8.74 (s, 2H), 8.26 (s, |
| | 1H), 7.98 (s, 1H), 7.84-7.60 (m, 3H), 7.58-7.24 (m, 6H), 6.29 (s, 1H), 4.66-4.44 (m, 1H), |
| | 3.17-2.99 (m, 2H), 2.29-2.09 (m, 4H). |
| TDH-315 | 1H NMR (400 MHz, DMSO-d6) δ 10.91 (s, 1H), 9.04-8.90 (m, 1H), 8.72 (s, 2H), 8.24 (s, |
| | 1H), 7.98 (s, 1H), 7.85-7.68 (m, 3H), 7.58-7.38 (m, 4H), 7.38-7.22 (m, 2H), 7.20-7.06 |
| | (m, 1H), 6.27 (s, 1H), 4.62-4.49 (m, 1H), 3.15-2.98 (m, 2H), 2.28-2.05 (m, 4H). |
| TDH-316 | 1H NMR (400 MHz, DMSO-d6) δ 10.65 (s, 1H), 9.05-8.91 (m, 1H), 8.81-8.65 (m, 2H), |
| | 8.24 (s, 1H), 7.99 (s, 1H), 7.74 (d, J = 7.6 Hz, 1H), 7.58-7.38 (m, 4H), 7.25-7.14 (m, 2H), |
| | 6.65 (d, J = 8.5 Hz, 1H), 6.27 (s, 1H), 4.62-4.50 (m, 1H), 3.70 (s, 3H), 3.15-3.02 (m, 2H), |
| | 2.29-2.11 (m, 4H). |
| TDH-317 | 1H NMR (300 MHz, DMSO-d6) δ 10.50 (s, 1H), 8.94 (s, 1H), 8.72 (s, 2H), 8.25 (s, 1H), |
| | 7.99 (s, 1H), 7.73 (d, J = 8.0 Hz, 2H), 7.55-7.38 (m, 5H), 6.88 (d, J = 8.0 Hz, 2H), 6.26 (s, |
| | 1H), 4.61-4.49 (m, 1H), 3.71 (s, 3H), 3.16-3.01 (m, 2H), 2.28-2.13 (m, 4H). |
| TDH-318 | 1H NMR (400 MHz, DMSO-d6) δ 10.57 (s, 1H), 9.05-8.94 (m, 1H), 8.82-8.67 (m, 2H), |
| | 8.25 (s, 1H), 8.00 (s, 1H), 7.74 (d, J = 7.6 Hz, 2H), 7.53-7.35 (m, 5H), 7.19 (t, J = 7.9 Hz, |
| | 1H), 6.89 (d, J = 7.6 Hz, 1H), 6.28 (s, 1H), 4.61-4.51 (m, 1H), 3.15-3.03 (m, 2H), 2.30- |
| | 2.14 (m, 7H). |
| TDH-319 | 1H NMR (400 MHz, DMSO-d6) δ 10.55 (s, 1H), 9.06-8.91 (m, 1H), 8.81-8.66 (m, 2H), |
| | 8.25 (s, 1H), 7.99 (s, 1H), 7.73 (d, J = 7.5 Hz, 2H), 7.58-7.38 (m, 5H), 7.31 (s, 2H), 7.11 |
| | (d, J = 8.1 Hz, 2H), 6.28 (s, 1H), 4.62-4.49 (m, 1H), 3.15-3.02 (m, 2H), 2.30-2.10 (m, 7H). |
| TDH-320 | 1H NMR (300 MHz, DMSO-d6) δ 10.70 (s, 1H), 9.08-8.97 (m, 1H), 8.86-8.74 (m, 1H), |
| | 8.72 (s, 1H), 8.25 (s, 1H), 7.98 (s, 1H), 7.66-7.56 (m, 4H), 7.29 (d, J = 8.0 Hz, 2H), 7.19- |
| | 7.09 (m, 2H), 6.22 (s, 1H), 4.61-4.50 (m, 1H), 3.47-3.37 (m, 2H), 3.17-2.99 (m, 2H), 2.33 |
| | (s, 3H), 2.28-2.10 (m, 4H). LC/MS (ESI) m/z 530.5 [M + H]+ |
| TDH-321 | 1H NMR (300 MHz, DMSO-d6) δ 10.86 (s, 1H), 9.04-8.92 (m, 1H), 8.80-8.66 (m, 2H), |
| | 8.28 (s, 1H), 8.01 (s, 1H), 7.87-7.82 (m, 1H), 7.75-7.68 (m, 1H), 7.67-7.59 (m, 2H), |
| | 7.59-7.47 (m, 2H), 7.21-7.12 (m, 2H), 6.28 (s, 1H), 4.61-4.52 (m, 1H), 3.46-3.38 (m, |
| | 2H), 3.18-3.00 (m, 2H), 2.29-2.12 (m, 4H). LC/MS (ESI) m/z 550.5 [M + H] |
| TDH-322 | 1H NMR (300 MHz, DMSO-d6) δ 10.86 (s, 1H), 9.10-8.97 (m, 1H), 8.89-8.78 (m, 1H), |
| | 8.75 (s, 1H), 8.27 (s, 1H), 8.02 (s, 1H), 7.83-7.75 (m, 2H), 7.68-7.55 (m, 4H), 7.32 (s, |
| | 2H), 7.22-7.12 (m, 2H), 6.30 (s, 1H), 4.63-4.54 (m, 1H), 3.48-3.35 (m, 2H), 3.18-3.00 |
| | (m, 2H), 2.29-2.13 (m, 4H). LC/MS (ESI) m/z 550.5 [M + H] |
| TDH-323 | 1H NMR (300 MHz, CDCl3) δ 8.87 (d, J = 5.4 Hz, 1H), 8.50 (s, 1H), 7.99 (s, 1H), 7.86 (s, |
| | 1H), 7.57-7.48 (m, 4H), 7.23 (d, J = 7.9 Hz, 2H), 7.05-6.95 (m, 2H), 6.39 (s, 1H), 6.33 (s, |
| | 2H), 4.83-4.72 (m, 1H), 4.45-4.31 (m, 1H), 4.03-3.92 (m, 1H), 3.32-3.18 (m, 1H), 2.83- |
| | 2.69 (m, 1H), 2.36 (s, 3H), 2.30-2.16 (m, 2H), 2.15 (d, J = 2.0 Hz, 3H), 2.05-1.88 (m, 2H). |
| | LC/MS (ESI) m/z 572.6 [M + H]+ |
| TDH-324 | 1H NMR (400 MHz, CDCl3) δ 8.88 (d, J = 6.0 Hz, 1H), 8.53 (s, 1H), 7.99 (s, 1H), 7.89 (s, |
| | 1H), 7.65 (s, 1H), 7.59-7.48 (m, 3H), 7.40-7.34 (m, 2H), 7.00 (t, J = 8.4 Hz, 2H), 6.44- |
| | 6.27 (m, 3H), 4.84-4.73 (m, 1H), 4.46-4.33 (m, 1H), 4.05-3.92 (m, 1H), 3.33-3.19 (m, |
| | 1H), 2.83-2.71 (m, 1H), 2.30-2.18 (m, 2H), 2.15 (s, 3H), 2.07-1.90 (m, 2H). LC/MS (ESI) |
| | m/z 592.5 [M + H]+ |
| TDH-325 | 1H NMR (400 MHz, CDCl3) δ 8.91 (d, J = 7.1 Hz, 1H), 8.52 (s, 1H), 7.99 (s, 1H), 7.86 (s, |
| | 1H), 7.57 (d, J = 8.1 Hz, 2H), 7.55-7.48 (m, 2H), 7.43-7.36 (m, 2H), 7.05-6.96 (m, 2H), |
| | 6.38 (s, 1H), 6.33 (s, 2H), 4.82-4.74 (m, 1H), 4.44-4.33 (m, 1H), 4.03-3.93 (m, 1H), |
| | 3.32-3.19 (m, 1H), 2.83-2.71 (m, 1H), 2.31-2.16 (m, 2H), 2.14 (d, J = 3.2 Hz, 3H), 2.05- |
| | 1.89 (m, 2H). LC/MS (ESI) m/z 592.6 [M + H]+ |
| TDH-326 | 1H NMR (500 MHz, DMSO-d6) δ 10.69 (s, 1H), 9.10-8.96 (m, 1H), 8.86-8.75 (m, 1H), |
| | 8.72 (s, 1H), 8.25 (s, 1H), 7.99 (s, 1H), 7.67-7.58 (m, 4H), 7.33 (s, 2H), 7.16 (t, J = 8.6 |
| | Hz, 2H), 7.05 (d, J = 8.4 Hz, 2H), 6.21 (s, 1H), 4.59-4.52 (m, 1H), 3.44-3.37 (m, 2H), |
| | 3.14-3.03 (m, 2H), 2.27-2.11 (m, 4H). LC/MS (ESI) m/z 546.6 [M + H]+ |
| TDH-327 | 1H NMR (500 MHz, DMSO-d6) δ 10.66 (s, 1H), 8.99 (s, 1H), 8.08-8.03 (m, 2H), 7.76- |
| | 7.69 (m, 2H), 7.63-7.59 (m, 2H), 7.58-7.54 (m, 3H), 7.53-7.47 (m, 3H), 7.46-7.41 (m, |
| | 1H), 7.16 (t, J = 8.7 Hz, 2H), 6.26 (s, 1H). |
| TDH-328 | ¹H NMR (500 MHz, DMSO-d₆) δ 11.13 (s, 1H), 10.63 (s, 1H), 8.69 (s, 1H), 8.30 (s, 1H), |
| | 7.91 (s, 1H), 7.88-7.80 (m, 2H), 7.79-7.68 (m, 3H), 7.67-7.57 (m, 2H), 7.54-7.39 (m, |
| | 3H), 7.30 (s, 2H), 7.21-7.11 (m, 2H), 6.23 (s, 1H), 5.20-5.09 (m, 1H), 4.43-4.32 (m, 1H), |
| | 4.25-4.07 (m, 1H), 3.93-3.81 (m, 1H), 3.03-2.97 (m, 2H), 2.94-2.84 (m, 3H), 2.82-2.65 |
| | (m, 2H), 2.65-2.52 (m, 4H), 2.20-2.09 (m, 2H), 2.07-1.89 (m, 7H), 1.79-1.65 (m, 3H), |
| | 0.95-0.84 (m, 2H). |
| TDH-329 | 1H NMR (500 MHz, DMSO-d6) δ 10.69 (s, 1H), 9.10 (s, 1H), 8.29 (d, J = 8.2 Hz, 2H), 8.04 |
| | (d, J = 8.1 Hz, 2H), 7.75 (d, J = 7.7 Hz, 2H), 7.70-7.60 (m, 3H), 7.55-7.49 (m, 2H), 7.47- |
| | 7.42 (m, 1H), 7.20-7.13 (m, 2H), 6.28 (s, 1H), 3.27 (s, 3H). LC/MS (ESI) m/z 521.4 [M + H]+ |
| TDH-330 | ¹H NMR (400 MHz, DMSO) δ 11.19 (s, 1H), 10.63 (s, 1H), 8.69 (s, 1H), 8.29 (s, 1H), |
| | 8.23-8.16 (m, 1H), 7.92 (s, 1H), 7.76-7.69 (m, 3H), 7.63-7.59 (m, 2H), 7.55 (s, 1H), |
| | 7.51-7.47 (m, 2H), 7.45-7.40 (m, 1H), 7.29 (s, 2H), 7.18-7.13 (m, 2H), 6.23 (s, 1H), |
| | 6.00-5.89 (m, 1H), 5.29-5.06 (m, 2H), 4.37-4.28 (m, 1H), 3.81-3.72 (m, 1H), 3.02-2.89 |
| | (m, 4H), 2.76-2.59 (m, 3H), 2.40-2.33 (m, 2H), 2.31-2.23 (m, 1H), 2.09-1.97 (m, 7H), |
| | 1.75-1.67 (m, 2H), 1.33-1.13 (m, 3H), 1.05-0.90 (m, 2H). |
| TDH-331 | ¹H NMR (400 MHz, DMSO) δ 11.12 (s, 1H), 10.65 (s, 1H), 8.70 (s, 1H), 8.29 (s, 1H), 7.93 |
| | (s, 1H), 7.81-7.69 (m, 3H), 7.67-7.57 (m, 2H), 7.54-7.39 (m, 4H), 7.37-7.25 (m, 3H), |
| | 7.21-7.10 (m, 2H), 6.24 (s, 1H), 5.18 (s, 2H), 5.14-5.06 (m, 1H), 4.27-4.13 (m, 1H), |
| | 3.85-3.74 (m, 1H), 3.70-3.49 (m, 4H), 3.28-3.20 (m, 1H), 3.20-3.14 (m, 1H), 3.10-2.96 |
| | (m, 2H), 2.93-2.84 (m, 1H), 2.66-2.52 (m, 4H), 2.27-2.13 (m, 2H), 2.11-1.87 (m, 6H), |
| | 1.85-1.75 (m, 1H), 1.62-1.50 (m, 1H), 1.46-1.33 (m, 1H). |
| TDH-332 | ¹H NMR (400 MHz, DMSO) δ 11.12 (s, 1H), 10.66 (s, 1H), 8.70 (s, 1H), 8.29 (s, 1H), 7.93 |
| | (s, 1H), 7.86-7.79 (m, 1H), 7.76-7.69 (m, 2H), 7.66-7.57 (m, 2H), 7.53-7.46 (m, 2H), |
| | 7.46-7.40 (m, 2H), 7.36-7.27 (m, 3H), 7.20-7.11 (m, 2H), 6.24 (s, 1H), 5.17-5.06 (m, |
| | 3H), 4.26-4.07 (m, 1H), 3.86-3.76 (m, 1H), 3.69-3.51 (m, 4H), 3.26-3.19 (m, 1H), 3.18- |
| | 3.13 (m, 1H), 3.08-2.94 (m, 2H), 2.93-2.84 (m, 1H), 2.65-2.52 (m, 4H), 2.28-2.12 (m, |
| | 2H), 2.11-1.87 (m, 6H), 1.85-1.77 (m, 1H), 1.59-1.48 (m, 1H), 1.44-1.32 (m, 1H). |
| TDH-333 | ¹H NMR (400 MHz, DMSO) δ 11.15 (s, 1H), 10.67 (s, 1H), 8.70 (s, 1H), 8.29 (s, 1H), |
| | 8.03-7.97 (m, 1H), 7.93 (s, 1H), 7.76-7.70 (m, 2H), 7.66-7.58 (m, 2H), 7.54-7.46 (m, |
| | 2H), 7.46-7.39 (m, 1H), 7.34-7.25 (m, 2H), 7.20-7.10 (m, 3H), 6.90 (s, 1H), 6.24 (s, 1H), |
| | 5.92-5.83 (m, 1H), 4.42-4.34 (m, 1H), 4.34-4.23 (m, 2H), 4.21-4.13 (m, 2H), 3.97-3.86 |
| | (m, 1H), 3.68-3.57 (m, 1H), 3.07-2.89 (m, 4H), 2.70-2.56 (m, 3H), 2.42-2.30 (m, 2H), |
| | 2.25-2.19 (m, 1H), 2.17-1.81 (m, 7H), 1.78-1.68 (m, 2H), 1.61-1.53 (m, 1H), 1.49-1.37 |
| | (m, 2H), 1.18-1.08 (m, 1H), 1.06-0.96 (m, 1H). |
| TDH-334 | ¹H NMR (400 MHz, DMSO) δ 11.14 (s, 1H), 10.65 (s, 1H), 8.70 (s, 1H), 8.29 (s, 1H), |
| | 7.97-7.88 (m, 2H), 7.77-7.69 (m, 2H), 7.66-7.58 (m, 2H), 7.54-7.46 (m, 3H), 7.46-7.39 |
| | (m, 1H), 7.35-7.28 (m, 2H), 7.28-7.21 (m, 1H), 7.20-7.11 (m, 3H), 6.24 (s, 1H), 5.91- |
| | 5.82 (m, 1H), 4.43-4.32 (m, 1H), 4.25-4.08 (m, 3H), 4.00-3.91 (m, 1H), 3.10-2.90 (m, |
| | 4H), 2.71-2.60 (m, 3H), 2.43-2.26 (m, 2H), 2.26-2.18 (m, 1H), 2.15-1.87 (m, 6H), 1.81- |
| | 1.69 (m, 2H), 1.65-1.54 (m, 1H), 1.50-1.36 (m, 2H), 1.25-1.14 (m, 1H), 1.06-0.95 (m, 1H). |
| TDH-335 | 1H NMR (300 MHz, DMSO) δ 10.66 (s, 1H), 9.11 (s, 1H), 8.69-8.65 (m, 2H), 8.02-7.97 |
| | (m, 2H), 7.78-7.69 (m, 4H), 7.66-7.59 (m, 2H), 7.54-7.43 (m, 3H), 7.22-7.12 (m, 2H), |
| | 6.27 (s, 1H). |
| TDH-336 | ¹H NMR (300 MHz, DMSO) δ 11.14 (s, 1H), 10.64 (s, 1H), 8.69 (s, 1H), 8.30 (s, 1H), 7.91 |
| | (s, 1H), 7.80-7.69 (m, 5H), 7.67-7.56 (m, 2H), 7.54-7.40 (m, 3H), 7.31 (s, 2H), 7.16 (t, J = |
| | 8.9 Hz, 2H), 6.23 (s, 1H), 5.20-5.09 (m, 1H), 4.43-4.32 (m, 1H), 4.24-4.14 (m, 1H), |
| | 3.92-3.80 (m, 1H), 3.30-3.21 (m, 2H), 3.19-3.16 (m, 3H), 2.99-2.83 (m, 4H), 2.71-2.64 |
| | (m, 2H), 2.19-2.12 (m, 2H), 2.08-1.92 (m, 7H), 1.77-1.66 (m, 3H), 0.97-0.83 (m, 2H). |
| TDH-337 | ¹H NMR (400 MHz, DMSO) δ 11.11 (s, 1H), 10.64 (s, 1H), 8.70 (s, 1H), 8.30 (s, 1H), 7.94 |
| | (s, 1H), 7.81-7.68 (m, 3H), 7.68-7.56 (m, 2H), 7.56-7.39 (m, 4H), 7.39-7.24 (m, 3H), |
| | 7.15 (t, J = 8.9 Hz, 2H), 6.24 (s, 1H), 5.26-5.03 (m, 3H), 4.61-4.42 (m, 2H), 4.34-4.21 (m, |
| | 1H), 4.09-3.95 (m, 1H), 3.84-3.72 (m, 1H), 3.25-3.13 (m, 1H), 3.11-2.98 (m, 1H), 2.98- |
| | 2.83 (m, 1H), 2.80-2.69 (m, 1H), 2.69-2.55 (m, 3H), 2.40-2.27 (m, 2H), 2.13-1.92 (m, |
| | 4H), 1.92-1.81 (m, 1H), 1.82-1.68 (m, 3H), 1.33-1.22 (m, 1H), 1.15-0.97 (m, 1H). |
| TDH-338 | ¹H NMR (400 MHz, DMSO) δ 11.12 (s, 1H), 10.63 (s, 1H), 8.70 (s, 1H), 8.30 (s, 1H), 7.94 |
| | (s, 1H), 7.87-7.80 (m, 1H), 7.75-7.68 (m, 2H), 7.65-7.57 (m, 2H), 7.52-7.46 (m, 2H), |
| | 7.46-7.39 (m, 2H), 7.37-7.25 (m, 3H), 7.16 (t, J = 9.3 Hz, 2H), 6.23 (s, 1H), 5.20-5.03 (m, |
| | 3H), 4.58-4.43 (m, 2H), 4.34-4.23 (m, 1H), 4.07-3.97 (m, 1H), 3.85-3.73 (m, 1H), 3.24- |
| | 3.15 (m, 1H), 3.11-3.01 (m, 1H), 2.92-2.84 (m, 1H), 2.78-2.70 (m, 1H), 2.67-2.56 (m, |
| | 3H), 2.36-2.30 (m, 2H), 2.12-1.97 (m, 4H), 1.89-1.80 (m, 1H), 1.80-1.67 (m, 3H), 1.27- |
| | 1.17 (m, 1H), 1.11-0.97 (m, 1H). |
| TDH-339 | ¹H NMR (400 MHz, DMSO) δ 11.11 (s, 1H), 10.64 (s, 1H), 8.73-8.63 (m, 1H), 8.29 (s, |
| | 1H), 7.94 (s, 1H), 7.76-7.68 (m, 2H), 7.65-7.56 (m, 3H), 7.53-7.46 (m, 2H), 7.46-7.40 |
| | (m, 1H), 7.33-7.26 (m, 2H), 7.19-7.06 (m, 5H), 6.24 (s, 1H), 5.13-5.01 (m, 1H), 4.53- |
| | 4.44 (m, 2H), 4.42-4.32 (m, 1H), 4.23-4.12 (m, 2H), 4.06-3.97 (m, 1H), 3.94-3.89 (m, |
| | 1H), 3.23-3.16 (m, 1H), 3.09-2.99 (m, 1H), 2.93-2.85 (m, 1H), 2.77-2.67 (m, 2H), 2.62- |
| | 2.56 (m, 2H), 2.35-2.31 (m, 2H), 2.10-2.00 (m, 4H), 1.91-1.84 (m, 1H), 1.78-1.70 (m, |
| | 3H), 1.26-1.18 (m, 1H), 1.10-1.00 (m, 1H). |
| TDH-340 | ¹H NMR (400 MHz, DMSO) δ 11.07 (s, 1H), 10.63 (s, 1H), 8.70 (s, 1H), 8.29 (s, 1H), 7.94 |
| | (s, 1H), 7.76-7.69 (m, 2H), 7.66-7.53 (m, 3H), 7.53-7.46 (m, 2H), 7.46-7.39 (m, 1H), |
| | 7.35-7.24 (m, 2H), 7.20-7.11 (m, 3H), 7.11-6.98 (m, 2H), 6.23 (s, 1H), 5.09-4.99 (m, |
| | 1H), 4.57-4.43 (m, 2H), 4.41-4.31 (m, 1H), 4.18-4.07 (m, 2H), 4.07-3.98 (m, 1H), 3.98- |
| | 3.89 (m, 1H), 3.24-3.15 (m, 1H), 3.09-2.99 (m, 1H), 2.92-2.83 (m, 1H), 2.76-2.54 (m, |
| | 4H), 2.38-2.31 (m, 2H), 2.12-2.04 (m, 2H), 2.03-1.93 (m, 2H), 1.90-1.83 (m, 1H), 1.81- |
| | 1.70 (m, 3H), 1.25-1.17 (m, 1H), 1.09-0.99 (m, 1H). |
| TDH-341 | ¹H NMR (400 MHz, DMSO) δ 11.09 (s, 1H), 10.64 (s, 1H), 8.69 (s, 1H), 8.30 (s, 1H), 7.94 |
| | (s, 1H), 7.77-7.64 (m, 3H), 7.64-7.56 (m, 2H), 7.53-7.46 (m, 2H), 7.46-7.39 (m, 1H), |
| | 7.38-7.25 (m, 4H), 7.21-7.10 (m, 2H), 6.23 (s, 1H), 5.13-5.04 (m, 1H), 4.56-4.43 (m, |
| | 2H), 4.43-4.32 (m, 1H), 4.07-3.95 (m, 2H), 3.77-3.65 (m, 2H), 3.25-3.14 (m, 2H), 3.11- |
| | 3.00 (m, 1H), 3.00-2.91 (m, 2H), 2.91-2.81 (m, 2H), 2.80-2.70 (m, 1H), 2.63-2.55 (m, |
| | 2H), 2.37-2.28 (m, 2H), 2.15-1.96 (m, 4H), 1.87-1.66 (m, 8H), 1.20-1.09 (m, 1H), 1.09- |
| | 0.96 (m, 1H). |
| TDH-342 | ¹H NMR (400 MHz, DMSO) δ 11.08 (s, 1H), 10.64 (s, 1H), 8.70 (s, 1H), 8.29 (s, 1H), 7.94 |
| | (s, 1H), 7.78-7.70 (m, 2H), 7.70-7.56 (m, 3H), 7.54-7.46 (m, 2H), 7.46-7.39 (m, 1H), |
| | 7.36-7.28 (m, 3H), 7.28-7.20 (m, 1H), 7.20-7.11 (m, 2H), 6.24 (s, 1H), 5.12-5.00 (m, |
| | 1H), 4.59-4.44 (m, 2H), 4.41-4.27 (m, 1H), 4.09-3.99 (m, 4H), 3.22-3.13 (m, 2H), 3.11- |
| | 3.01 (m, 3H), 2.99-2.83 (m, 2H), 2.78-2.71 (m, 1H), 2.62-2.56 (m, 2H), 2.39-2.26 (m, |
| | 2H), 2.13-1.96 (m, 4H), 1.87-1.55 (m, 8H), 1.19-1.07 (m, 1H), 1.07-0.92 (m, 1H). |
| TDH-343 | ¹H NMR (400 MHz, DMSO) δ 11.09 (s, 1H), 10.65 (s, 1H), 8.70 (s, 1H), 8.30 (s, 1H), 7.93 |
| | (s, 1H), 7.77-7.70 (m, 2H), 7.70-7.64 (m, 1H), 7.64-7.58 (m, 2H), 7.52-7.46 (m, 2H), |
| | 7.46-7.39 (m, 1H), 7.38-7.28 (m, 4H), 7.20-7.12 (m, 2H), 6.24 (s, 1H), 5.14-5.05 (m, |
| | 1H), 4.58-4.45 (m, 2H), 4.09-4.00 (m, 1H), 3.74-3.64 (m, 2H), 3.28-3.17 (m, 1H), 2.95- |
| | 2.85 (m, 3H), 2.80-2.71 (m, 1H), 2.64-2.56 (m, 2H), 2.43-2.34 (m, 2H), 2.16-2.02 (m, |
| | 3H), 2.02-1.98 (m, 1H), 1.87-1.69 (m, 4H), 1.51-1.38 (m, 2H). |
| TDH-344 | ¹H NMR (400 MHz, DMSO) δ 11.08 (s, 1H), 10.64 (s, 1H), 8.69 (s, 1H), 8.30 (s, 1H), 7.93 |
| | (s, 1H), 7.75-7.69 (m, 2H), 7.67-7.57 (m, 3H), 7.52-7.46 (m, 2H), 7.46-7.39 (m, 1H), |
| | 7.36-7.28 (m, 3H), 7.27-7.21 (m, 1H), 7.19-7.12 (m, 2H), 6.24 (s, 1H), 5.11-5.02 (m, |
| | 1H), 4.57-4.43 (m, 2H), 4.10-3.99 (m, 3H), 3.25-3.14 (m, 1H), 3.05-2.95 (m, 2H), 2.93- |
| | 2.83 (m, 1H), 2.80-2.71 (m, 1H), 2.63-2.57 (m, 2H), 2.37-2.30 (m, 2H), 2.13-1.99 (m, |
| | 4H), 1.90-1.74 (m, 4H), 1.28-1.22 (m, 2H). |
| TDH-345 | ¹H NMR (400 MHz, DMSO) δ 11.13 (s, 1H), 10.63 (s, 1H), 8.69 (s, 1H), 8.30 (s, 1H), |
| | 8.01-7.89 (m, 2H), 7.88-7.78 (m, 1H), 7.77-7.69 (m, 2H), 7.66-7.58 (m, 2H), 7.55-7.46 |
| | (m, 3H), 7.46-7.38 (m, 2H), 7.35-7.22 (m, 2H), 7.22-7.11 (m, 2H), 6.24 (s, 1H), 5.18- |
| | 5.07 (m, 1H), 4.79 (s, 2H), 4.26-4.10 (m, 1H), 3.07-2.98 (m, 2H), 2.98-2.83 (m, 3H), |
| | 2.64-2.54 (m, 2H), 2.20-1.89 (m, 9H), 1.86-1.66 (m, 4H), 1.51-1.33 (m, 2H), 1.02-0.75 |
| | (m, 4H). |
| TDH-346 | ¹H NMR (400 MHz, DMSO) δ 11.12 (s, 1H), 10.63 (s, 1H), 8.69 (s, 1H), 8.30 (s, 1H), 8.20 |
| | (s, 1H), 7.95-7.83 (m, 2H), 7.76-7.70 (m, 2H), 7.65-7.58 (m, 2H), 7.53-7.37 (m, 5H), |
| | 7.32-7.24 (m, 2H), 7.20-7.09 (m, 2H), 6.24 (s, 1H), 5.17-5.07 (m, 1H), 4.74 (s, 2H), |
| | 4.24-4.10 (m, 1H), 3.04-2.96 (m, 2H), 2.95-2.81 (m, 3H), 2.65-2.54 (m, 2H), 2.13-1.88 |
| | (m, 9H), 1.83-1.73 (m, 2H), 1.73-1.64 (m, 2H), 1.48-1.33 (m, 2H), 0.95-0.75 (m, 4H). |
| TDH-347 | ¹H NMR (400 MHz, DMSO) δ 11.11 (s, 1H), 10.64 (s, 1H), 8.69 (s, 1H), 8.29 (s, 1H), |
| | 8.13-8.06 (m, 1H), 7.92 (s, 1H), 7.76-7.69 (m, 2H), 7.66-7.56 (m, 3H), 7.52-7.46 (m, |
| | 2H), 7.46-7.39 (m, 1H), 7.30 (s, 2H), 7.20-7.11 (m, 2H), 7.11-7.04 (m, 1H), 6.99-6.92 |
| | (m, 1H), 6.90-6.83 (m, 1H), 6.24 (s, 1H), 5.12-5.03 (m, 1H), 4.18 (s, 1H), 3.93 (s, 2H), |
| | 3.02-2.82 (m, 5H), 2.65-2.54 (m, 2H), 2.19-1.89 (m, 9H), 1.83-1.74 (m, 2H), 1.74-1.65 |
| | (m, 2H), 1.48-1.30 (m, 2H), 0.96-0.77 (m, 4H). |
| TDH-348 | ¹H NMR (400 MHz, DMSO) δ 11.07 (s, 1H), 10.65 (s, 1H), 8.69 (s, 1H), 8.29 (s, 1H), |
| | 8.08-8.01 (m, 1H), 7.92 (s, 1H), 7.76-7.70 (m, 2H), 7.66-7.56 (m, 3H), 7.54-7.45 (m, |
| | 2H), 7.45-7.34 (m, 2H), 7.30 (s, 2H), 7.20-7.11 (m, 2H), 6.94 (s, 1H), 6.91-6.83 (m, 1H), |
| | 6.24 (s, 1H), 5.09-4.99 (m, 1H), 4.17 (s, 1H), 3.85 (s, 2H), 3.00-2.81 (m, 5H), 2.62-2.53 |
| | (m, 2H), 2.17-1.88 (m, 9H), 1.82-1.73 (m, 2H), 1.72-1.64 (m, 2H), 1.44-1.30 (m, 2H), |
| | 0.95-0.76 (m, 4H). |
| TDH-349 | ¹H NMR (400 MHz, DMSO) δ 11.09 (s, 1H), 10.67 (s, 1H), 8.70 (s, 1H), 8.28 (s, 1H), 7.94 |
| | (s, 1H), 7.82 (s, 1H), 7.77-7.66 (m, 3H), 7.66-7.58 (m, 2H), 7.53-7.46 (m, 2H), 7.46-7.40 |
| | (m, 1H), 7.39-7.28 (m, 4H), 7.20-7.11 (m, 2H), 6.25 (s, 1H), 5.16-5.02 (m, 1H), 4.27- |
| | 4.10 (m, 1H), 3.78-3.67 (m, 2H), 3.03-2.84 (m, 7H), 2.63-2.55 (m, 2H), 2.41-2.27 (m, |
| | 2H), 2.18-1.91 (m, 7H), 1.90-1.62 (m, 9H), 1.42-1.28 (m, 1H), 1.27-1.12 (m, 1H), 1.03- |
| | 0.72 (m, 4H). |
| TDH-350 | ¹H NMR (400 MHz, DMSO) δ 11.08 (s, 1H), 10.69 (s, 1H), 8.71 (s, 1H), 8.27 (s, 1H), |
| | 8.03-7.88 (m, 1H), 7.83 (s, 1H), 7.77-7.70 (m, 2H), 7.70-7.57 (m, 3H), 7.54-7.37 (m, |
| | 3H), 7.37-7.21 (m, 4H), 7.21-7.09 (m, 2H), 6.25 (s, 1H), 5.14-4.97 (m, 1H), 4.12-4.01 |
| | (m, 2H), 3.70-3.54 (m, 1H), 3.06-2.81 (m, 7H), 2.63-2.54 (m, 2H), 2.34-2.19 (m, 2H), |
| | 2.14-1.90 (m, 5H), 1.90-1.50 (m, 10H), 1.44-1.27 (m, 2H), 1.28-1.14 (m, 1H), 1.05-0.80 |
| | (m, 4H). |
| TDH-351 | ¹H NMR (400 MHz, DMSO) δ 11.10 (s, 1H), 10.62 (s, 1H), 8.69 (s, 1H), 8.30 (s, 1H), 7.91 |
| | (s, 1H), 7.76-7.69 (m, 2H), 7.66-7.54 (m, 3H), 7.53-7.46 (m, 2H), 7.46-7.39 (m, 1H), |
| | 7.29 (s, 2H), 7.20-7.08 (m, 3H), 7.05-6.99 (m, 1H), 6.62-6.54 (m, 1H), 6.23 (s, 1H), |
| | 5.10-5.01 (m, 1H), 4.23-4.13 (m, 1H), 3.24-3.13 (m, 2H), 2.97-2.85 (m, 3H), 2.65-2.56 |
| | (m, 2H), 2.18-2.10 (m, 2H), 2.03-1.91 (m, 7H), 1.84-1.74 (m, 4H), 1.58-1.42 (m, 2H), |
| | 1.06-0.95 (m, 2H), 0.91-0.78 (m, 2H). |
| TDH-352 | ¹H NMR (400 MHz, DMSO) δ 11.06 (s, 1H), 10.64 (s, 1H), 8.69 (s, 1H), 8.30 (s, 1H), 7.92 |
| | (s, 1H), 7.77-7.69 (m, 2H), 7.66-7.59 (m, 2H), 7.59-7.53 (m, 1H), 7.53-7.46 (m, 2H), |
| | 7.46-7.38 (m, 1H), 7.30 (s, 2H), 7.23-7.10 (m, 3H), 6.96 (s, 1H), 6.92-6.84 (m, 1H), 6.24 |
| | (s, 1H), 5.08-4.99 (m, 1H), 4.28-4.13 (m, 1H), 3.06-2.99 (m, 2H), 2.94-2.82 (m, 3H), |
| | 2.61-2.55 (m, 2H), 2.07-1.90 (m, 9H), 1.87-1.78 (m, 4H), 1.55-1.46 (m, 2H), 1.05-0.95 |
| | (m, 2H), 0.91-0.84 (m, 2H). |
| TDH-353 | ¹H NMR (400 MHz, DMSO) δ 11.07 (s, 1H), 10.64 (s, 1H), 8.70 (s, 1H), 8.31 (s, 1H), |
| | 7.98-7.91 (m, 1H), 7.76-7.69 (m, 2H), 7.66-7.54 (m, 3H), 7.53-7.39 (m, 3H), 7.34-7.27 |
| | (m, 2H), 7.20-7.06 (m, 4H), 7.06-6.98 (m, 1H), 6.24 (s, 1H), 5.08-5.00 (m, 1H), 4.56- |
| | 4.46 (m, 2H), 4.44-4.34 (m, 1H), 4.23-4.10 (m, 3H), 4.02-3.92 (m, 1H), 3.19-3.16 (m, |
| | 1H), 3.06-2.97 (m, 1H), 2.93-2.82 (m, 2H), 2.80-2.72 (m, 2H), 2.64-2.55 (m, 2H), 2.16- |
| | 1.95 (m, 3H), 1.94-1.84 (m, 1H), 1.78-1.57 (m, 4H), 1.49-1.35 (m, 1H). |
| TDH-354 | 1H NMR (400 MHz, DMSO-d6) δ 11.11 (s, 1H), 10.64 (s, 1H), 8.69 (s, 1H), 8.33 (s, 1H), |
| | 7.93 (s, 1H), 7.89-7.67 (m, 6H), 7.64-7.58 (m, 2H), 7.52-7.34 (m, 6H), 7.30 (s, 2H), |
| | 7.19-7.12 (m, 2H), 6.24 (s, 1H), 5.23 (s, 2H), 5.16-5.10 (m, 1H), 4.55 (s, 2H), 4.48-4.40 |
| | (m, 2H), 3.99-3.89 (m, 1H), 3.29-3.05 (m, 3H), 2.97-2.83 (m, 2H), 2.78-2.70 (m, 1H), |
| | 2.65-2.55 (m, 2H), 2.17-1.98 (m, 7H), 1.91 (s, 2H). |
| TDH-355 | ¹H NMR (400 MHz, DMSO-d₆) δ 11.11 (s, 1H), 10.63 (s, 1H), 8.69 (s, 1H), 8.33 (s, 1H), |
| | 7.93 (s, 1H), 7.88-7.67 (m, 6H), 7.64-7.58 (m, 2H), 7.52-7.34 (m, 6H), 7.30 (s, 2H), |
| | 7.20-7.11 (m, 2H), 6.23 (s, 1H), 5.17 (s, 2H), 5.14-5.08 (m, 1H), 4.55 (s, 1H), 4.47-4.43 |
| | (m, 1H), 4.00-3.92 (m, 1H), 3.25-3.14 (m, 3H), 2.96-2.83 (m, 2H), 2.80-2.69 (m, 2H), |
| | 2.65-2.56 (m, 2H), 2.16-2.08 (m, 3H), 2.04 (d, J = 12.0 Hz, 4H), 1.88 (s, 1H). |
| TDH-356 | ¹H NMR (400 MHz, DMSO-d₆) δ 11.12 (s, 1H), 10.65 (s, 1H), 8.69 (s, 1H), 8.32 (s, 1H), |
| | 7.99 (s, 1H), 7.93 (s, 1H), 7.89 (s, 1H), 7.72 (d, J = 7.6 Hz, 2H), 7.67-7.57 (m, 3H), 7.53- |
| | 7.46 (m, 2H), 7.46-7.38 (m, 1H), 7.30 (s, 2H), 7.19-7.09 (m, 4H), 7.08 (s, 1H), 6.24 (s, |
| | 1H), 5.13-5.03 (m, 1H), 4.59-4.52 (m, 2H), 4.47-4.40 (m, 1H), 4.26-4.22 (m, 2H), 4.11- |
| | 4.03 (m, 1H), 3.23-3.15 (m, 3H), 2.95-2.84 (m, 2H), 2.83-2.75 (m, 1H), 2.60-2.54 (m, |
| | 2H), 2.16-2.09 (m, 3H), 2.08-2.03 (m, 2H), 1.93-1.89 (m, 1H). |
| TDH-357 | ¹H NMR (400 MHz, DMSO) δ 11.07 (s, 1H), 10.66 (s, 1H), 8.69 (s, 1H), 8.29 (s, 1H), 7.92 |
| | (s, 1H), 7.79-7.69 (m, 2H), 7.66-7.55 (m, 3H), 7.53-7.39 (m, 3H), 7.30 (s, 2H), 7.20-7.06 |
| | (m, 4H), 7.06-6.97 (m, 1H), 6.24 (s, 1H), 5.10-4.99 (m, 1H), 4.26-4.17 (m, 1H), 4.13 (s, |
| | 2H), 3.56-3.50 (m, 3H), 3.05-2.94 (m, 2H), 2.94-2.81 (m, 1H), 2.62-2.55 (m, 2H), 2.47- |
| | 2.29 (m, 8H), 2.29-1.82 (m, 8H), 1.71-1.58 (m, 2H). |
| TDH-358 | ¹H NMR (400 MHz, DMSO) δ 11.12 (s, 1H), 10.67 (s, 1H), 8.70 (s, 1H), 8.29 (s, 1H), 7.92 |
| | (s, 1H), 7.88-7.82 (m, 1H), 7.78-7.70 (m, 2H), 7.67-7.58 (m, 2H), 7.53-7.46 (m, 2H), |
| | 7.46-7.39 (m, 2H), 7.38-7.27 (m, 3H), 7.21-7.11 (m, 2H), 6.24 (s, 1H), 5.21-5.08 (m, |
| | 3H), 4.28-4.14 (m, 1H), 3.49-3.44 (m, 3H), 3.04-2.82 (m, 3H), 2.65-2.56 (m, 2H), 2.46- |
| | 2.26 (m, 8H), 2.21-1.82 (m, 8H), 1.72-1.58 (m, 2H). |
| TDH-359 | ¹H NMR (400 MHz, DMSO-H₆) δ 11.12 (s, 1H), 10.64 (s, 1H), 8.70 (s, 1H), 8.28 (s, 1H), |
| | 7.94 (s, 1H), 7.86-7.76 (m, 1H), 7.76-7.69 (m, 2H), 7.60 (s, 2H), 7.53-7.25 (m, 7H), |
| | 7.18-7.05 (m, 2H), 6.23 (s, 1H), 5.18-5.05 (m, 3H), 5.01-4.89 (m, 1H), 4.33-4.21 (m, |
| | 1H), 3.82-3.72 (m, 1H), 3.06-2.95 (m, 1H), 2.95-2.76 (m, 4H), 2.64-2.54 (m, 4H), 2.43- |
| | 2.26 (m, 3H), 2.15-2.00 (m, 2H), 1.85-1.67 (m, 3H), 1.21-1.11 (m, 1H), 1.00-0.87 (m, 1H). |
| TDH-360 | ¹H NMR (400 MHz, DMSO) δ 11.07 (s, 1H), 10.65 (s, 1H), 8.72 (s, 1H), 8.38 (s, 1H), 7.99 |
| | (s, 1H), 7.77-7.70 (m, 2H), 7.65-7.54 (m, 3H), 7.54-7.46 (m, 2H), 7.46-7.39 (m, 1H), |
| | 7.33 (s, 2H), 7.20-7.11 (m, 3H), 7.11-7.04 (m, 1H), 7.04-6.97 (m, 1H), 6.24 (s, 1H), |
| | 5.12-5.01 (m, 2H), 4.41-4.31 (m, 1H), 4.11 (s, 2H), 3.99-3.88 (m, 1H), 3.80-3.67 (m, |
| | 2H), 3.46-3.37 (m, 2H), 3.09-2.98 (m, 1H), 2.95-2.80 (m, 1H), 2.72-2.54 (m, 3H), 2.45- |
| | 2.35 (m, 2H), 2.05-1.93 (m, 1H), 1.81-1.67 (m, 2H), 1.65-1.53 (m, 1H), 1.21-1.10 (m, |
| | 1H), 1.03-0.90 (m, 1H). |
| TDH-361 | ¹H NMR (400 MHz, DMSO-d₆) δ 11.07 (s, 1H), 10.64 (s, 1H), 8.69 (s, 1H), 8.32 (s, 1H), |
| | 7.92 (s, 1H), 7.87-7.81 (m, 3H), 7.76-7.69 (m, 3H), 7.65-7.55 (m, 4H), 7.53-7.46 (m, |
| | 3H), 7.46-7.37 (m, 2H), 7.30 (s, 2H), 7.20-7.13 (m, 4H), 7.10 (s, 1H), 7.10-7.03 (m, 2H), |
| | 6.23 (s, 1H), 5.10-5.01 (m, 2H), 4.57-4.52 (m, 2H), 4.44-4.40 (m, 1H), 4.20-4.16 (m, |
| | 2H), 4.16-4.08 (m, 2H), 3.19-3.15 (m, 2H), 2.94-2.82 (m, 2H), 2.81-2.70 (m, 2H), 2.62- |
| | 2.58 (m, 2H), 2.57-2.52 (m, 4H), 2.16-2.08 (m, 3H), 2.06-2.02 (m, 3H), 1.92-1.88 (m, 2H). |
| TDH-362 | ¹H NMR (400 MHz, DMSO-d₆) δ 11.10 (s, 1H), 10.63 (s, 1H), 8.69 (s, 1H), 8.32 (s, 1H), |
| | 7.92 (s, 1H), 7.85 (s, 2H), 7.75-7.66 (m, 3H), 7.64-7.58 (m, 2H), 7.52-7.45 (m, 2H), |
| | 7.44-7.39 (m, 1H), 7.38-7.28 (m, 4H), 7.19-7.11 (m, 2H), 6.23 (s, 1H), 5.14-5.07 (m, |
| | 1H), 4.59-4.54 (m, 1H), 4.43-4.35 (m, 1H), 4.21-4.13 (m, 1H), 3.77-3.69 (m, 2H), 3.18- |
| | 3.14 (m, 1H), 3.04-2.83 (m, 6H), 2.67-2.55 (m, 4H), 2.16-1.99 (m, 6H), 1.94-1.73 (m, 9H). |
| TDH-363 | ¹H NMR (400 MHz, DMSO-d₆) δ 11.09 (s, 1H), 10.64 (s, 1H), 8.69 (s, 1H), 8.32 (s, 1H), |
| | 7.93 (s, 1H), 7.85 (s, 2H), 7.74-7.59 (m, 5H), 7.51-7.40 (m, 3H), 7.35-7.23 (m, 4H), |
| | 7.19-7.12 (m, 2H), 6.23 (s, 1H), 5.12-5.03 (m, 1H), 4.57-4.53 (m, 1H), 4.39 (s, 1H), |
| | 4.23-4.15 (m, 1H), 4.11-4.04 (m, 2H), 3.22-3.00 (m, 5H), 2.97-2.82 (m, 2H), 2.69-2.54 |
| | (m, 4H), 2.16-1.97 (m, 6H), 1.95-1.57 (m, 10H). |
| TDH-364 | ¹H NMR (400 MHz, DMSO-d₆) δ 11.10 (s, 1H), 10.64 (s, 1H), 8.72-8.67 (m, 1H), 8.36- |
| | 8.31 (m, 1H), 7.95-7.88 (m, 2H), 7.85 (s, 1H), 7.76-7.69 (m, 3H), 7.65-7.57 (m, 2H), |
| | 7.52-7.35 (m, 6H), 7.30 (s, 1H), 7.20-7.11 (m, 2H), 6.23 (s, 1H), 5.16-5.07 (m, 1H), |
| | 4.63-4.48 (m, 2H), 4.41-4.31 (m, 1H), 3.89-3.81 (m, 2H), 3.11-3.03 (m, 2H), 2.91-2.84 |
| | (m, 1H), 2.64-2.54 (m, 3H), 2.17-2.00 (m, 7H), 1.99-1.77 (m, 4H). |
| TDH-365 | 1H NMR (400 MHz, DMSO-d6) δ 11.09 (s, 1H), 10.63 (s, 1H), 8.71-8.66 (m, 1H), 8.32 (s, |
| | 1H), 7.92 (s, 1H), 7.88-7.81 (m, 2H), 7.75-7.65 (m, 3H), 7.64-7.57 (m, 2H), 7.52-7.39 |
| | (m, 5H), 7.36-7.28 (m, 3H), 7.20-7.11 (m, 2H), 6.23 (s, 1H), 5.12-5.03 (m, 1H), 4.57- |
| | 4.52 (m, 1H), 4.47-4.42 (m, 1H), 4.27-4.19 (m, 2H), 3.15-3.04 (m, 3H), 2.91-2.83 (m, |
| | 2H), 2.69-2.59 (m, 3H), 2.15-2.07 (m, 4H), 2.00-1.97 (m, 2H), 1.97-1.87 (m, 4H). |
| TDH-366 | ¹H NMR (400 MHz, DMSO) δ 11.09 (s, 1H), 10.63 (s, 1H), 8.69 (s, 1H), 8.29 (s, 1H), 7.92 |
| | (s, 1H), 7.75-7.58 (m, 5H), 7.53-7.46 (m, 2H), 7.46-7.39 (m, 1H), 7.37-7.27 (m, 4H), |
| | 7.21-7.11 (m, 2H), 6.24 (s, 1H), 5.13-5.06 (m, 1H), 4.44-4.35 (m, 1H), 4.24-4.13 (m, |
| | 1H), 3.95-3.86 (m, 1H), 3.73-3.63 (m, 2H), 3.03-2.83 (m, 6H), 2.64-2.55 (m, 2H), 2.39- |
| | 2.25 (m, 4H), 2.13-1.87 (m, 9H), 1.80-1.64 (m, 4H), 1.59-1.50 (m, 1H), 1.44-1.33 (m, |
| | 4H), 1.10-1.02 (m, 1H), 0.99-0.90 (m, 1H). |
| TDH-367 | ¹H NMR (400 MHz, DMSO) δ 11.08 (s, 1H), 10.63 (s, 1H), 8.69 (s, 1H), 8.29 (s, 1H), 7.91 |
| | (s, 1H), 7.75-7.70 (m, 2H), 7.67-7.58 (m, 3H), 7.52-7.45 (m, 2H), 7.45-7.39 (m, 1H), |
| | 7.35-7.26 (m, 3H), 7.26-7.19 (m, 1H), 7.19-7.11 (m, 2H), 6.23 (s, 1H), 5.10-5.02 (m, |
| | 1H), 4.44-4.33 (m, 1H), 4.19 (s, 1H), 4.06-4.00 (m, 2H), 3.91-3.82 (m, 1H), 3.03-2.82 |
| | (m, 6H), 2.64-2.54 (m, 2H), 2.39-2.29 (m, 2H), 2.29-2.22 (m, 2H), 2.09-1.91 (m, 9H), |
| | 1.78-1.66 (m, 4H), 1.59-1.50 (m, 1H), 1.26-1.12 (m, 4H), 1.06-0.98 (m, 1H), 0.94-0.86 |
| | (m, 1H). |
| TDH-368 | ¹H NMR (400 MHz, DMSO) δ 11.09 (s, 1H), 10.68 (s, 1H), 8.70 (s, 1H), 8.29 (s, 1H), 7.92 |
| | (s, 1H), 7.77-7.70 (m, 2H), 7.70-7.58 (m, 3H), 7.55-7.46 (m, 2H), 7.46-7.39 (m, 1H), |
| | 7.36-7.27 (m, 4H), 7.20-7.11 (m, 2H), 6.25 (s, 1H), 5.14-5.04 (m, 1H), 4.26-4.14 (m, |
| | 1H), 3.73-3.63 (m, 2H), 3.04-2.93 (m, 2H), 2.93-2.81 (m, 4H), 2.63-2.53 (m, 2H), 2.43- |
| | 2.24 (m, 11H), 2.17-1.84 (m, 10H), 1.81-1.73 (m, 2H), 1.66-1.56 (m, 2H), 1.45-1.36 (m, 2H). |
| TDH-369 | ¹H NMR (400 MHz, DMSO) δ 11.08 (s, 1H), 10.66 (s, 1H), 8.70 (s, 1H), 8.29 (s, 1H), 7.92 |
| | (s, 1H), 7.77-7.70 (m, 2H), 7.67-7.58 (m, 3H), 7.54-7.46 (m, 2H), 7.46-7.39 (m, 1H), |
| | 7.34-7.26 (m, 3H), 7.26-7.19 (m, 1H), 7.19-7.11 (m, 2H), 6.24 (s, 1H), 5.12-5.02 (m, |
| | 1H), 4.25-4.13 (m, 1H), 4.08-3.98 (m, 2H), 3.04-2.83 (m, 6H), 2.63-2.54 (m, 2H), 2.40- |
| | 2.24 (m, 11H), 2.17-2.07 (m, 3H), 2.05-1.90 (m, 7H), 1.78-1.72 (m, 2H), 1.66-1.60 (m, |
| | 2H), 1.29-1.14 (m, 2H). |
| TDH-370 | ¹H NMR (400 MHz, DMSO) δ 11.20 (s, 1H), 10.68 (s, 1H), 8.70 (s, 1H), 8.30 (s, 1H), |
| | 8.23-8.17 (m, 1H), 7.92 (s, 1H), 7.76-7.67 (m, 3H), 7.65-7.58 (m, 2H), 7.58-7.55 (m, |
| | 1H), 7.53-7.46 (m, 2H), 7.46-7.39 (m, 1H), 7.34-7.25 (m, 2H), 7.20-7.10 (m, 2H), 6.24 |
| | (s, 1H), 5.98-5.90 (m, 1H), 5.19 (s, 2H), 4.26-4.16 (m, 1H), 3.06-2.92 (m, 3H), 2.73-2.63 |
| | (m, 2H), 2.47-2.41 (m, 2H), 2.41-2.20 (m, 8H), 2.16-1.90 (m, 8H), 1.72-1.55 (m, 3H). |
| TDH-371 | ¹H NMR (400 MHz, DMSO) δ 11.10 (s, 1H), 10.64 (s, 1H), 8.69 (s, 1H), 8.29 (s, 1H), 7.91 |
| | (s, 1H), 7.76-7.69 (m, 2H), 7.69-7.56 (m, 3H), 7.52-7.45 (m, 2H), 7.45-7.38 (m, 1H), |
| | 7.37-7.26 (m, 4H), 7.20-7.10 (m, 2H), 6.23 (s, 1H), 5.13-5.04 (m, 1H), 4.23-4.11 (m, |
| | 1H), 3.94-3.82 (m, 1H), 3.75-3.62 (m, 3H), 3.62-3.50 (m, 3H), 3.26-3.18 (m, 1H), 3.12- |
| | 3.04 (m, 1H), 3.04-2.94 (m, 2H), 2.92-2.81 (m, 3H), 2.64-2.53 (m, 3H), 2.36-2.27 (m, |
| | 2H), 2.25-2.12 (m, 2H), 2.07-1.96 (m, 4H), 1.96-1.89 (m, 2H), 1.89-1.82 (m, 2H), 1.81- |
| | 1.72 (m, 3H), 1.44-1.29 (m, 4H). |
| TDH-372 | ¹H NMR (400 MHz, DMSO) δ 11.09 (s, 1H), 10.64 (s, 1H), 8.69 (s, 1H), 8.29 (s, 1H), 7.92 |
| | (s, 1H), 7.76-7.69 (m, 2H), 7.67-7.57 (m, 3H), 7.54-7.45 (m, 2H), 7.45-7.38 (m, 1H), |
| | 7.35-7.27 (m, 3H), 7.27-7.19 (m, 1H), 7.19-7.09 (m, 2H), 6.23 (s, 1H), 5.11-5.02 (m, |
| | 1H), 4.23-4.11 (m, 1H), 4.09-3.98 (m, 2H), 3.92-3.82 (m, 1H), 3.74-3.63 (m, 1H), 3.62- |
| | 3.48 (m, 3H), 3.25-3.13 (m, 1H), 3.11-3.03 (m, 1H), 3.03-2.82 (m, 5H), 2.64-2.53 (m, |
| | 3H), 2.30-2.24 (m, 2H), 2.24-2.13 (m, 2H), 2.06-1.88 (m, 6H), 1.86-1.69 (m, 4H), 1.45- |
| | 1.35 (m, 1H), 1.35-1.27 (m, 1H), 1.27-1.13 (m, 3H). |
| TDH-373 | ¹H NMR (400 MHz, DMSO) δ 11.20 (s, 1H), 10.63 (s, 1H), 8.69 (s, 1H), 8.29 (s, 1H), |
| | 8.23-8.17 (m, 1H), 7.92 (s, 1H), 7.78-7.66 (m, 3H), 7.66-7.58 (m, 2H), 7.58-7.54 (m, |
| | 1H), 7.54-7.46 (m, 2H), 7.46-7.37 (m, 1H), 7.38-7.26 (m, 2H), 7.20-7.11 (m, 2H), 6.23 |
| | (s, 1H), 6.00-5.90 (m, 1H), 5.19 (s, 2H), 4.23-4.12 (m, 1H), 3.85-3.76 (m, 1H), 3.71-3.62 |
| | (m, 1H), 3.62-3.51 (m, 3H), 3.30-3.21 (m, 1H), 3.21-3.09 (m, 1H), 3.05-2.89 (m, 3H), |
| | 2.74-2.62 (m, 2H), 2.61-2.53 (m, 2H), 2.32-2.14 (m, 3H), 2.08-1.89 (m, 5H), 1.85-1.75 |
| | (m, 1H), 1.59-1.49 (m, 1H), 1.42-1.33 (m, 1H). |
| TDH-374 | ¹H NMR (400 MHz, DMSO) δ 11.09 (s, 1H), 10.65 (s, 1H), 8.70 (s, 1H), 8.31 (s, 1H), 7.94 |
| | (s, 1H), 7.77-7.65 (m, 3H), 7.65-7.57 (m, 2H), 7.54-7.46 (m, 2H), 7.46-7.39 (m, 1H), |
| | 7.38-7.26 (m, 4H), 7.21-7.10 (m, 2H), 6.23 (s, 1H), 5.15-5.05 (m, 1H), 4.58-4.37 (m, |
| | 3H), 4.20-4.09 (m, 1H), 4.02-3.91 (m, 1H), 3.74-3.61 (m, 2H), 3.28-3.19 (m, 1H), 3.17- |
| | 3.06 (m, 1H), 3.03-2.93 (m, 1H), 2.92-2.81 (m, 3H), 2.79-2.70 (m, 1H), 2.70-2.53 (m, |
| | 3H), 2.36-2.29 (m, 2H), 2.18-1.96 (m, 4H), 1.96-1.83 (m, 2H), 1.83-1.73 (m, 3H), 1.72- |
| | 1.63 (m, 2H), 1.47-1.35 (m, 3H). |
| TDH-375 | ¹H NMR (400 MHz, DMSO) δ 11.09 (s, 1H), 10.64 (s, 1H), 8.70 (s, 1H), 8.30 (s, 1H), 7.94 |
| | (s, 1H), 7.76-7.67 (m, 2H), 7.68-7.56 (m, 3H), 7.55-7.39 (m, 3H), 7.39-7.28 (m, 3H), |
| | 7.28-7.20 (m, 1H), 7.20-7.06 (m, 2H), 6.23 (s, 1H), 5.11-5.03 (m, 1H), 4.57-4.46 (m, |
| | 2H), 4.46-4.37 (m, 1H), 4.20-4.09 (m, 1H), 4.09-3.99 (m, 2H), 3.95-3.85 (m, 1H), 3.28- |
| | 3.19 (m, 1H), 3.13-3.04 (m, 1H), 3.04-2.92 (m, 3H), 2.92-2.80 (m, 1H), 2.79-2.70 (m, |
| | 1H), 2.70-2.54 (m, 3H), 2.34-2.20 (m, 2H), 2.17-1.97 (m, 4H), 1.90-1.82 (m, 1H), 1.82- |
| | 1.71 (m, 3H), 1.71-1.60 (m, 2H), 1.56-1.44 (m, 1H), 1.44-1.31 (m, 1H), 1.26-1.17 (m, 2H). |
| TDH-376 | ¹H NMR (400 MHz, DMSO) δ 11.13 (s, 1H), 10.64 (s, 1H), 8.70 (s, 1H), 8.28 (s, 1H), 7.93 |
| | (s, 1H), 7.81-7.69 (m, 5H), 7.66-7.58 (m, 2H), 7.53-7.46 (m, 2H), 7.46-7.39 (m, 1H), |
| | 7.30 (s, 2H), 7.21-7.11 (m, 2H), 6.24 (s, 1H), 5.18-5.08 (m, 1H), 4.42-4.31 (m, 1H), 4.19 |
| | (s, 1H), 3.90-3.77 (m, 1H), 3.29-3.20 (m, 2H), 3.01-2.81 (m, 4H), 2.74-2.54 (m, 5H), |
| | 2.38-2.29 (m, 1H), 2.13-1.96 (m, 7H), 1.72-1.62 (m, 2H), 1.60-1.47 (m, 2H), 1.47-1.33 |
| | (m, 2H), 1.05-0.87 (m, 2H). |
| TDH-377 | ¹H NMR (400 MHz, DMSO) δ 11.13 (s, 1H), 10.67 (s, 1H), 8.70 (s, 1H), 8.28 (s, 1H), 7.95 |
| | (s, 1H), 7.90-7.80 (m, 2H), 7.80-7.69 (m, 3H), 7.69-7.59 (m, 2H), 7.54-7.40 (m, 3H), |
| | 7.31 (s, 2H), 7.24-7.10 (m, 2H), 6.25 (s, 1H), 5.23-5.09 (m, 1H), 4.42-4.31 (m, 1H), 4.20 |
| | (s, 1H), 3.92-3.79 (m, 1H), 3.07-2.85 (m, 6H), 2.78-2.70 (m, 2H), 2.66-2.55 (m, 2H), |
| | 2.12-1.92 (m, 7H), 1.72-1.63 (m, 2H), 1.58-1.47 (m, 2H), 1.45-1.30 (m, 2H), 1.05-0.80 |
| | (m, 4H). |
| TDH-378 | ¹H NMR (400 MHz, DMSO) δ 11.13 (s, 1H), 10.64 (s, 1H), 8.69 (s, 1H), 8.28 (s, 1H), 7.93 |
| | (s, 1H), 7.82-7.68 (m, 5H), 7.66-7.57 (m, 2H), 7.52-7.46 (m, 2H), 7.46-7.38 (m, 1H), |
| | 7.30 (s, 2H), 7.19-7.10 (m, 2H), 6.24 (s, 1H), 5.18-5.03 (m, 1H), 4.19 (s, 1H), 3.90-3.77 |
| | (m, 1H), 3.67-3.60 (m, 1H), 3.56-3.47 (m, 2H), 3.27-3.15 (m, 5H), 3.12-2.83 (m, 6H), |
| | 2.75-2.67 (m, 2H), 2.63-2.55 (m, 2H), 2.23-2.12 (m, 2H), 2.10-2.01 (m, 3H), 1.83-1.74 |
| | (m, 2H), 1.45-1.27 (m, 3H). |
| TDH-379 | ¹H NMR (400 MHz, DMSO) δ 11.12 (s, 1H), 10.63 (s, 1H), 8.69 (s, 1H), 8.28 (s, 1H), 7.92 |
| | (s, 1H), 7.87-7.79 (m, 2H), 7.79-7.68 (m, 3H), 7.65-7.57 (m, 2H), 7.55-7.40 (m, 3H), |
| | 7.30 (s, 2H), 7.22-7.11 (m, 2H), 6.24 (s, 1H), 5.18-5.06 (m, 1H), 4.18 (s, 1H), 3.88-3.76 |
| | (m, 1H), 3.70-3.61 (m, 1H), 3.59-3.50 (m, 2H), 3.31-3.13 (m, 4H), 3.12-3.03 (m, 2H), |
| | 3.03-2.83 (m, 5H), 2.79-2.69 (m, 2H), 2.65-2.56 (m, 2H), 2.23-2.10 (m, 2H), 2.09-2.00 |
| | (m, 3H), 1.84-1.75 (m, 2H), 1.44-1.27 (m, 3H). |
| TDH-380 | ¹H NMR (400 MHz, DMSO) δ 11.12 (s, 1H), 10.63 (s, 1H), 8.69 (s, 1H), 8.30 (s, 1H), 7.93 |
| | (s, 1H), 7.80-7.69 (m, 5H), 7.66-7.57 (m, 2H), 7.54-7.39 (m, 3H), 7.30 (s, 2H), 7.21-7.12 |
| | (m, 2H), 6.23 (s, 1H), 5.18-5.09 (m, 1H), 4.55-4.45 (m, 2H), 4.42-4.35 (m, 1H), 4.16- |
| | 4.09 (m, 1H), 3.92-3.84 (m, 1H), 3.11-3.03 (m, 2H), 3.02-2.83 (m, 4H), 2.69-2.57 (m, |
| | 4H), 2.14-2.05 (m, 3H), 1.94-1.83 (m, 2H), 1.83-1.71 (m, 2H), 1.69-1.61 (m, 2H), 1.52- |
| | 1.45 (m, 1H), 1.39-1.32 (m, 1H). |
| TDH-381 | ¹H NMR (400 MHz, DMSO) δ 11.12 (s, 1H), 10.63 (s, 1H), 8.69 (s, 1H), 8.30 (s, 1H), |
| | 7.97-7.91 (m, 1H), 7.89-7.80 (m, 2H), 7.80-7.70 (m, 3H), 7.65-7.57 (m, 2H), 7.52-7.38 |
| | (m, 3H), 7.30 (s, 2H), 7.23-7.09 (m, 2H), 6.24 (s, 1H), 5.18-5.09 (m, 1H), 4.57-4.44 (m, |
| | 2H), 4.44-4.31 (m, 1H), 4.18-4.08 (m, 1H), 3.96-3.84 (m, 1H), 3.02-2.88 (m, 4H), 2.82- |
| | 2.67 (m, 4H), 2.64-2.56 (m, 2H), 2.13-2.03 (m, 3H), 1.93-1.80 (m, 2H), 1.80-1.69 (m, |
| | 2H), 1.66-1.59 (m, 2H), 1.49-1.40 (m, 1H), 1.37-1.30 (m, 1H). |
| TDH-382 | ¹H NMR (400 MHz, DMSO) δ 11.12 (s, 1H), 10.63 (s, 1H), 8.69 (s, 1H), 8.29 (s, 1H), 7.92 |
| | (s, 1H), 7.80-7.69 (m, 5H), 7.65-7.58 (m, 2H), 7.52-7.38 (m, 3H), 7.29 (s, 2H), 7.19-7.09 |
| | (m, 2H), 6.24 (s, 1H), 5.18-5.06 (m, 1H), 4.25-4.12 (m, 1H), 3.49-3.39 (m, 4H), 3.28- |
| | 3.23 (m, 2H), 3.03-2.83 (m, 4H), 2.76-2.52 (m, 5H), 2.33-2.28 (m, 5H), 2.12-1.90 (m, |
| | 8H), 1.66-1.56 (m, 2H). |
| TDH-383 | ¹H NMR (400 MHz, DMSO) δ 11.12 (s, 1H), 10.64 (s, 1H), 8.69 (s, 1H), 8.28 (s, 1H), 7.92 |
| | (s, 1H), 7.88-7.81 (m, 2H), 7.79-7.70 (m, 3H), 7.66-7.57 (m, 2H), 7.54-7.38 (m, 3H), |
| | 7.29 (s, 2H), 7.22-7.11 (m, 2H), 6.24 (s, 1H), 5.18-5.07 (m, 1H), 4.27-4.12 (m, 1H), |
| | 3.49-3.38 (m, 4H), 3.05-2.83 (m, 5H), 2.77-2.67 (m, 2H), 2.66-2.55 (m, 2H), 2.43-2.22 |
| | (m, 8H), 2.16-1.90 (m, 7H), 1.69-1.53 (m, 2H). |
| TDH-384 | ¹H NMR (400 MHz, DMSO) δ 11.00 (s, 1H), 10.64 (s, 1H), 8.69 (s, 1H), 8.29 (s, 1H), |
| | 7.97-7.89 (m, 1H), 7.75-7.71 (m, 1H), 7.65-7.55 (m, 3H), 7.55-7.38 (m, 6H), 7.30 (s, |
| | 2H), 7.20-7.09 (m, 2H), 6.24 (s, 1H), 5.20-5.08 (m, 1H), 4.58-4.48 (m, 1H), 4.42-4.32 |
| | (m, 2H), 4.24-4.13 (m, 1H), 3.91-3.79 (m, 1H), 3.00-2.79 (m, 7H), 2.76-2.54 (m, 5H), |
| | 2.17-1.91 (m, 8H), 1.80-1.64 (m, 3H), 0.91-0.78 (m, 2H). |
| TDH-385 | ¹H NMR (400 MHz, DMSO) δ 11.00 (s, 1H), 10.66 (s, 1H), 8.70 (s, 1H), 8.28 (s, 1H), 7.94 |
| | (s, 1H), 7.77-7.68 (m, 2H), 7.65-7.55 (m, 3H), 7.54-7.39 (m, 5H), 7.30 (s, 2H), 7.20-7.11 |
| | (m, 2H), 6.25 (s, 1H), 5.22-5.08 (m, 1H), 4.57-4.43 (m, 1H), 4.42-4.30 (m, 2H), 4.29- |
| | 4.02 (m, 1H), 3.91-3.74 (m, 1H), 3.20-2.86 (m, 6H), 2.80-2.54 (m, 4H), 2.50-2.36 (m, |
| | 3H), 2.37-2.24 (m, 1H), 2.14-1.89 (m, 5H), 1.70-1.58 (m, 2H), 1.58-1.43 (m, 2H), 1.43- |
| | 1.27 (m, 1H), 1.28-1.12 (m, 1H), 1.01-0.79 (m, 2H). |
| TDH-386 | ¹H NMR (400 MHz, DMSO) δ 11.00 (s, 1H), 10.67 (d, J = 12.0 Hz, 1H), 8.70 (s, 1H), 8.29 |
| | (s, 1H), 7.93 (s, 1H), 7.77-7.68 (m, 2H), 7.65-7.55 (m, 3H), 7.53-7.39 (m, 5H), 7.30 (s, |
| | 2H), 7.24-7.09 (m, 2H), 6.25 (s, 1H), 5.20-5.08 (m, 1H), 4.59-4.45 (m, 1H), 4.40-4.32 |
| | (m, 1H), 4.32-4.18 (m, 1H), 3.47-3.40 (m, 4H), 3.15-2.99 (m, 2H), 2.96-2.84 (m, 3H), |
| | 2.74-2.67 (m, 2H), 2.66-2.57 (m, 2H), 2.47-2.40 (m, 2H), 2.37-2.17 (m, 8H), 2.10-1.97 |
| | (m, 5H), 1.71-1.59 (m, 2H). |
| TDH-387 | ¹H NMR (400 MHz, DMSO) δ 11.00 (s, 1H), 10.67 (s, 1H), 8.70 (s, 1H), 8.27 (s, 1H), 7.96 |
| | (s, 1H), 7.80-7.72 (m, 2H), 7.66-7.55 (m, 3H), 7.54-7.39 (m, 5H), 7.31 (s, 2H), 7.22-7.10 |
| | (m, 2H), 6.25 (s, 1H), 5.20-5.06 (m, 1H), 4.52 (d, J = 17.1 Hz, 1H), 4.35 (d, J = 17.1 Hz, |
| | 1H), 3.90-3.73 (m, 2H), 3.69-3.47 (m, 4H), 3.21-3.06 (m, 3H), 2.99-2.81 (m, 4H), 2.74- |
| | 2.67 (m, 2H), 2.66-2.57 (m, 2H), 2.45-2.37 (m, 1H), 2.30-2.16 (m, 2H), 2.06-1.88 (m, |
| | 3H), 1.83-1.70 (m, 2H), 1.39-1.23 (m, 5H). |
| TDH-388 | ¹H NMR (400 MHz, DMSO) δ 10.99 (s, 1H), 10.64 (s, 1H), 8.69 (s, 1H), 8.30 (s, 1H), 7.93 |
| | (s, 1H), 7.80-7.70 (m, 2H), 7.64-7.55 (m, 3H), 7.55-7.40 (m, 5H), 7.30 (s, 2H), 7.21-7.09 |
| | (m, 2H), 6.23 (s, 1H), 5.20-5.08 (m, 1H), 4.58-4.43 (m, 3H), 4.43-4.31 (m, 2H), 4.17- |
| | 4.05 (m, 1H), 3.96-3.83 (m, 1H), 3.29-3.18 (m, 1H), 3.09-3.00 (m, 1H), 2.97-2.82 (m, |
| | 4H), 2.79-2.69 (m, 3H), 2.69-2.60 (m, 2H), 2.14-1.98 (m, 3H), 1.91-1.80 (m, 1H), 1.78- |
| | 1.70 (m, 1H), 1.70-1.57 (m, 2H), 1.53-1.44 (m, 1H), 1.41-1.30 (m, 1H), 1.29-1.22 (m, 1H). |
| TDH-389 | ¹H NMR (500 MHz, DMSO) δ 11.45 (s, 1H), 11.13 (s, 1H), 10.89-10.77 (m, 2H), 8.74 (s, |
| | 1H), 8.27 (s, 1H), 8.01 (s, 1H), 7.89-7.83 (m, 1H), 7.78-7.73 (m, 2H), 7.66-7.61 (m, 2H), |
| | 7.54-7.49 (m, 3H), 7.46-7.37 (m, 3H), 7.20-7.13 (m, 2H), 6.29 (s, 1H), 5.27-5.16 (m, |
| | 2H), 5.16-5.10 (m, 1H), 4.61-4.51 (m, 1H), 4.46-4.37 (m, 1H), 4.08-3.99 (m, 1H), 3.72- |
| | 3.66 (m, 3H), 3.30-3.19 (m, 7H), 3.20-3.11 (m, 3H), 3.04-2.97 (m, 1H), 2.95-2.86 (m, |
| | 1H), 2.66-2.54 (m, 2H), 2.43-2.36 (m, 2H), 2.36-2.25 (m, 4H), 2.10-2.02 (m, 1H). |
| TDH-390 | ¹H NMR (400 MHz, DMSO-d₆) δ 11.11 (s, 1H), 10.65 (s, 1H), 8.69 (s, 1H), 8.28 (s, 1H), |
| | 7.93 (s, 1H), 7.88-7.81 (m, 1H), 7.80-7.67 (m, 2H), 7.67-7.56 (m, 2H), 7.54-7.41 (m, |
| | 4H), 7.41-7.35 (m, 1H), 7.35-7.25 (m, 2H), 7.21-7.09 (m, 2H), 6.24 (s, 1H), 5.31-5.19 |
| | (m, 1H), 5.19-5.02 (m, 2H), 4.33-4.09 (m, 2H), 3.99-3.81 (m, 2H), 3.81-3.65 (m, 2H), |
| | 3.28-3.15 (m, 2H), 3.06-2.76 (m, 4H), 2.64-2.57 (m, 2H), 2.30-2.16 (m, 2H), 2.16-1.84 |
| | (m, 6H). |
| TDH-391 | ¹H NMR (400 MHz, DMSO) δ 11.11 (s, 1H), 10.81-10.75 (m, 1H), 10.48 (s, 1H), 8.73 (s, |
| | 1H), 8.26 (s, 1H), 8.00 (s, 1H), 7.78-7.73 (m, 2H), 7.66-7.56 (m, 4H), 7.54-7.46 (m, 3H), |
| | 7.44-7.40 (m, 1H), 7.20-7.05 (m, 6H), 6.28 (s, 1H), 5.12-5.05 (m, 1H), 4.59-4.48 (m, |
| | 1H), 4.38 (s, 1H), 4.22-4.14 (m, 2H), 3.67-3.61 (m, 2H), 3.18-2.98 (m, 6H), 2.92-2.85 |
| | (m, 1H), 2.75-2.54 (m, 4H), 2.37-2.25 (m, 4H), 2.08-2.02 (m, 1H), 1.74-1.65 (m, 4H), |
| | 1.24-1.16 (m, 1H), 1.10-1.01 (m, 1H). |
| TDH-392 | ¹H NMR (400 MHz, DMSO) δ 11.12 (s, 1H), 10.79-10.74 (m, 1H), 10.39 (s, 1H), 8.76- |
| | 8.72 (m, 1H), 8.26 (s, 1H), 8.02-7.99 (m, 1H), 7.88-7.81 (m, 1H), 7.77-7.72 (m, 2H), |
| | 7.66-7.61 (m, 2H), 7.55-7.46 (m, 3H), 7.46-7.38 (m, 3H), 7.38-7.32 (m, 2H), 7.19-7.13 |
| | (m, 2H), 6.28 (s, 1H), 5.20-5.06 (m, 4H), 4.58-4.48 (m, 1H), 4.35-4.26 (m, 1H), 3.23- |
| | 3.02 (m, 6H), 2.94-2.86 (m, 1H), 2.69-2.55 (m, 3H), 2.38-2.26 (m, 4H), 2.11-2.03 (m, |
| | 1H), 1.80-1.60 (m, 6H), 1.28-1.18 (m, 1H), 1.09-0.98 (m, 1H). |
| TDH-393 | ¹H NMR (400 MHz, DMSO-d6) δ 11.10 (s, 1H), 10.63 (s, 1H), 8.70 (s, 1H), 8.29 (s, 1H), |
| | 7.94 (s, 1H), 7.79-7.69 (m, 3H), 7.62-7.57 (m, 2H), 7.50-7.41 (m, 5H), 7.33-7.28 (m, |
| | 2H), 7.20-7.09 (m, 2H), 6.24 (s, 1H), 5.18 (s, 2H), 5.14-5.07 (m, 1H), 4.54-4.39 (m, 2H), |
| | 4.28-4.21 (m, 2H), 4.13-4.05 (m, 1H), 4.00-3.89 (m, 1H), 3.68-3.58 (m, 2H), 3.23-3.12 |
| | (m, 4H), 2.65-2.56 (m, 2H), 2.13-1.99 (m, 4H), 1.94 (s, 2H), 1.83 (s, 2H), 1.60 (s, 1H), |
| | 1.43 (s, 1H). |
| TDH-394 | ¹H NMR (400 MHz, DMSO-d6) δ 11.11 (s, 1H), 10.63 (s, 1H), 8.70 (s, 1H), 8.28 (s, 1H), |
| | 7.94 (s, 1H), 7.82 (d, J = 8.3 Hz, 1H), 7.72 (d, J = 7.6 Hz, 2H), 7.61 (d, J = 7.9 Hz, 2H), |
| | 7.56-7.40 (m, 5H), 7.37-7.28 (m, 2H), 7.15 (t, J = 7.8 Hz, 2H), 6.23 (s, 1H), 5.76 (s, 1H), |
| | 5.13 (s, 2H), 4.57-4.39 (m, 2H), 4.24 (s, 2H), 4.00-3.89 (m, 1H), 3.85-3.77 (m, 1H), |
| | 3.68-3.59 (m, 2H), 3.25-3.12 (m, 4H), 2.66-2.55 (m, 2H), 2.15-2.01 (m, 4H), 1.99-1.89 |
| | (m, 2H), 1.88-1.79 (m, 2H), 1.63-1.51 (m, 1H), 1.48-1.34 (m, 1H). |
| TDH-395 | ¹H NMR (400 MHz, DMSO-d6) δ 11.10 (s, 1H), 10.63 (s, 1H), 8.69 (s, 1H), 8.28 (s, 1H), |
| | 7.94 (s, 1H), 7.75-7.69 (m, 2H), 7.64-7.57 (m, 3H), 7.50-7.46 (m, 2H), 7.45-7.39 (m, |
| | 1H), 7.30 (s, 2H), 7.18-7.06 (m, 5H), 6.24 (s, 1H), 5.12-5.02 (m, 1H), 4.55-4.48 (m, 1H), |
| | 4.46-4.39 (m, 1H), 4.25 (s, 2H), 4.19 (s, 2H), 3.98-3.86 (m, 2H), 3.69-3.61 (m, 2H), |
| | 2.92-2.79 (m, 2H), 2.69-2.57 (m, 2H), 2.16-2.02 (m, 4H), 1.95-1.83 (m, 4H), 1.63-1.51 |
| | (m, 2H), 1.49-1.38 (m, 2H). |
| TDH-396 | ¹H NMR (400 MHz, DMSO-d6) δ 11.05 (s, 1H), 10.62 (s, 1H), 8.69 (s, 1H), 8.28 (s, 1H), |
| | 7.93 (s, 1H), 7.75-7.70 (m, 2H), 7.62-7.54 (m, 3H), 7.49-7.46 (m, 2H), 7.45-7.40 (m, |
| | 1H), 7.29 (s, 2H), 7.18-7.11 (m, 3H), 7.07-7.00 (m, 2H), 6.23 (s, 1H), 5.07-5.01 (m, 1H), |
| | 4.50 (s, 1H), 4.41 (s, 1H), 4.25 (s, 2H), 4.13 (s, 2H), 3.98-3.87 (m, 2H), 3.66-3.59 (m, |
| | 2H), 2.88-2.79 (m, 2H), 2.64-2.56 (m, 2H), 2.00-1.91 (m, 4H), 1.87-1.72 (m, 4H), 1.62- |
| | 1.48 (m, 2H), 1.46-1.34 (m, 2H). |
| TDH-397 | ¹H NMR (400 MHz, DMSO-d6) δ 11.08 (s, 1H), 10.63 (s, 1H), 8.69 (s, 1H), 8.29 (s, 1H), |
| | 7.94 (s, 1H), 7.76-7.65 (m, 3H), 7.63-7.57 (m, 2H), 7.53-7.39 (m, 4H), 7.36-7.29 (m, |
| | 3H), 7.19-7.09 (m, 2H), 6.24 (s, 1H), 5.15-5.04 (m, 1H), 4.24 (s, 2H), 4.00-3.92 (m, 1H), |
| | 3.91-3.84 (m, 1H), 3.81-3.75 (m, 1H), 3.75-3.68 (m, 2H), 3.24-3.04 (m, 4H), 3.03-2.91 |
| | (m, 2H), 2.90-2.68 (m, 4H), 2.64-2.55 (m, 2H), 2.13-1.98 (m, 4H), 1.96-1.87 (m, 2H), |
| | 1.86-1.71 (m, 6H), 1.43 (d, J = 40.8 Hz, 2H). |
| TDH-398 | ¹H NMR (400 MHz, DMSO-d6) δ 11.06 (s, 1H), 10.63 (s, 1H), 8.69 (s, 1H), 8.28 (s, 1H), |
| | 7.93 (s, 1H), 7.74-7.70 (m, 2H), 7.67-7.62 (m, 2H), 7.51-7.47 (m, 2H), 7.44-7.41 (m, |
| | 1H), 7.33-7.29 (m, 3H), 7.24-7.20 (m, 2H), 7.16-7.11 (m, 2H), 6.23 (s, 1H), 5.08-5.03 |
| | (m, 1H), 4.23 (s, 2H), 4.09-4.01 (m, 2H), 3.90-3.78 (m, 3H), 3.19-3.15 (m, 2H), 3.10- |
| | 3.03 (m, 4H), 2.91-2.78 (m, 4H), 2.65-2.59 (m, 2H), 2.14-2.04 (m, 4H), 1.84-1.77 (m, |
| | 2H), 1.71-1.57 (m, 6H), 1.48-1.36 (m, 2H). |
| TDH-399 | ¹H NMR (400 MHz, DMSO-d6) δ 11.09 (s, 1H), 10.64 (s, 1H), 8.77-8.65 (m, 1H), 8.30 (d, |
| | J = 2.8 Hz, 1H), 7.94 (s, 1H), 7.75-7.66 (m, 3H), 7.64-7.57 (m, 2H), 7.51-7.42 (m, 4H), |
| | 7.38-7.26 (m, 4H), 7.15 (t, J = 8.8 Hz, 2H), 6.23 (s, 1H), 5.10 (d, J = 12.7 Hz, 1H), 4.26 (s, |
| | 2H), 3.99 (d, J = 14.6 Hz, 1H), 3.53 (s, 2H), 3.27-3.15 (m, 1H), 3.09 (s, 2H), 2.86 (d, J = |
| | 14.8 Hz, 2H), 2.61 (s, 1H), 2.09 (s, 1H), 1.94 (s, OH), 1.70 (s, 2H). |
| TDH-400 | ¹H NMR (400 MHz, DMSO-d6) δ 11.08 (s, 1H), 10.65 (s, 1H), 8.69 (s, 1H), 8.29 (s, 1H), |
| | 7.93 (s, 1H), 7.74-7.70 (m, 2H), 7.69-7.58 (m, 3H), 7.52-7.42 (m, 3H), 7.35-7.23 (m, |
| | 4H), 7.19-7.11 (m, 2H), 6.24 (s, 1H), 5.11-5.03 (m, 1H), 4.26 (s, 2H), 4.02-3.91 (m, 1H), |
| | 3.73-3.62 (m, 2H), 3.30-3.15 (m, 4H), 2.92-2.76 (m, 3H), 2.54-2.54 (m, 2H), 2.19-2.03 |
| | (m, 4H), 2.01-1.91 (m, 4H), 1.61-1.51 (m, 2H). |
| TDH-401 | ¹H NMR (400 MHz, DMSO) δ 11.08 (s, 1H), 10.64 (s, 1H), 8.70 (s, 1H), 8.30 (s, 1H), 7.94 |
| | (s, 1H), 7.77-7.69 (m, 2H), 7.69-7.58 (m, 3H), 7.53-7.40 (m, 3H), 7.30 (s, 2H), 7.15 (t, J = |
| | 8.8 Hz, 2H), 6.87-6.79 (m, 1H), 6.73-6.65 (m, 1H), 6.24 (s, 1H), 5.12-5.01 (m, 1H), |
| | 4.59-4.45 (m, 2H), 4.42-4.32 (m, 1H), 4.30-4.18 (m, 2H), 4.17-4.08 (m, 2H), 4.08-3.95 |
| | (m, 1H), 3.95-3.84 (m, 1H), 3.66-3.58 (m, 1H), 3.27-3.18 (m, 1H), 3.09-2.97 (m, 1H), |
| | 2.92-2.82 (m, 1H), 2.80-2.72 (m, 1H), 2.70-2.54 (m, 3H), 2.39-2.29 (m, 2H), 2.15-1.97 |
| | (m, 4H), 1.91-1.81 (m, 1H), 1.81-1.68 (m, 3H), 1.21-1.00 (m, 2H). |
| TDH-402 | ¹H NMR (400 MHz, DMSO) δ 11.08 (s, 1H), 10.63 (s, 1H), 8.69 (s, 1H), 8.29 (s, 1H), 7.94 |
| | (s, 1H), 7.76-7.70 (m, 2H), 7.67-7.58 (m, 3H), 7.53-7.46 (m, 2H), 7.46-7.40 (m, 1H), |
| | 7.36-7.25 (m, 3H), 7.25-7.19 (m, 1H), 7.19-7.12 (m, 2H), 6.24 (s, 1H), 5.11-5.02 (m, |
| | 1H), 4.57-4.44 (m, 2H), 4.44-4.33 (m, 1H), 4.09-3.97 (m, 3H), 3.92-3.82 (m, 1H), 3.24- |
| | 3.14 (m, 1H), 3.02-2.91 (m, 3H), 2.91-2.83 (m, 1H), 2.79-2.71 (m, 1H), 2.63-2.54 (m, |
| | 3H), 2.36-2.29 (m, 2H), 2.28-2.23 (m, 2H), 2.14-1.93 (m, 6H), 1.79-1.70 (m, 4H), 1.29- |
| | 1.15 (m, 3H), 1.12-0.97 (m, 2H). |
| TDH-403 | ¹H NMR (400 MHz, DMSO) δ 11.12 (s, 1H), 10.64 (s, 1H), 8.69 (s, 1H), 8.29 (s, 1H), 7.94 |
| | (s, 1H), 7.85-7.80 (m, 2H), 7.78-7.70 (m, 3H), 7.64-7.58 (m, 2H), 7.52-7.46 (m, 2H), |
| | 7.45-7.40 (m, 1H), 7.30 (s, 2H), 7.20-7.12 (m, 2H), 6.24 (s, 1H), 5.20-5.10 (m, 1H), |
| | 4.57-4.44 (m, 2H), 4.40-4.30 (m, 1H), 4.05-3.95 (m, 1H), 3.91-3.81 (m, 1H), 3.24-3.14 |
| | (m, 1H), 3.05-2.83 (m, 5H), 2.78-2.69 (m, 3H), 2.63-2.53 (m, 3H), 2.32-2.25 (m, 2H), |
| | 2.13-2.02 (m, 3H), 1.87-1.79 (m, 1H), 1.78-1.73 (m, 1H), 1.72-1.65 (m, 2H), 1.06-0.92 |
| | (m, 2H). |
| TDH-404 | ¹H NMR (400 MHz, DMSO) δ 11.00 (s, 1H), 10.64 (s, 1H), 8.69 (s, 1H), 8.29 (s, 1H), 7.94 |
| | (s, 1H), 7.76-7.69 (m, 2H), 7.65-7.55 (m, 3H), 7.52-7.40 (m, 5H), 7.30 (s, 2H), 7.20-7.12 |
| | (m, 2H), 6.24 (s, 1H), 5.18-5.08 (m, 1H), 4.58-4.44 (m, 3H), 4.40-4.31 (m, 2H), 4.04- |
| | 3.94 (m, 1H), 3.89-3.77 (m, 1H), 3.24-3.18 (m, 1H), 3.00-2.83 (m, 4H), 2.80-2.55 (m, |
| | 5H), 2.46-2.37 (m, 1H), 2.30-2.24 (m, 2H), 2.11-1.96 (m, 3H), 1.91-1.72 (m, 3H), 1.72- |
| | 1.60 (m, 2H), 1.01-0.88 (m, 2H). |
| TDH-405 | ¹H NMR (400 MHz, DMSO) δ 11.13 (s, 1H), 10.63 (s, 1H), 8.70 (s, 1H), 8.30 (s, 1H), |
| | 7.98-7.86 (m, 2H), 7.78-7.67 (m, 2H), 7.67-7.56 (m, 2H), 7.53-7.46 (m, 3H), 7.46-7.40 |
| | (m, 1H), 7.30 (s, 2H), 7.28-7.22 (m, 1H), 7.18-7.07 (m, 3H), 6.24 (s, 1H), 5.91-5.81 (m, |
| | 1H), 4.57-4.45 (m, 2H), 4.42-4.33 (m, 1H), 4.23-4.08 (m, 2H), 4.08-3.98 (m, 1H), 3.98- |
| | 3.89 (m, 1H), 3.25-3.16 (m, 1H), 3.12-3.04 (m, 1H), 2.97-2.88 (m, 1H), 2.79-2.62 (m, |
| | 4H), 2.42-2.30 (m, 2H), 2.29-2.17 (m, 1H), 2.12-2.04 (m, 2H), 2.04-1.96 (m, 1H), 1.91- |
| | 1.84 (m, 1H), 1.82-1.70 (m, 3H), 1.29-1.19 (m, 1H), 1.12-0.98 (m, 1H). |
| TDH-406 | ¹H NMR (400 MHz, DMSO) δ 11.13 (s, 1H), 10.63 (s, 1H), 8.69 (s, 1H), 8.29 (s, 1H), |
| | 8.04-7.97 (m, 1H), 7.96-7.90 (m, 1H), 7.75-7.68 (m, 2H), 7.65-7.57 (m, 2H), 7.54-7.46 |
| | (m, 2H), 7.46-7.39 (m, 1H), 7.30 (s, 2H), 7.22-7.09 (m, 3H), 6.94-6.84 (m, 1H), 6.23 (s, |
| | 1H), 5.94-5.82 (m, 1H), 4.57-4.44 (m, 2H), 4.41-4.24 (m, 3H), 4.20-4.12 (m, 2H), 4.07- |
| | 3.97 (m, 1H), 3.97-3.87 (m, 1H), 3.67-3.57 (m, 1H), 3.25-3.14 (m, 1H), 3.09-2.91 (m, |
| | 2H), 2.80-2.73 (m, 1H), 2.70-2.58 (m, 3H), 2.36-2.29 (m, 2H), 2.26-2.19 (m, 1H), 2.14- |
| | 2.03 (m, 2H), 2.03-1.92 (m, 1H), 1.89-1.80 (m, 1H), 1.80-1.66 (m, 3H), 1.26-1.12 (m, |
| | 1H), 1.12-0.99 (m, 1H). |
| TDH-407 | ¹H NMR (400 MHz, DMSO) δ 11.15 (s, 1H), 10.64 (s, 1H), 8.70 (s, 1H), 8.29 (s, 1H), 8.01 |
| | (d, J = 9.1 Hz, 1H), 7.94 (s, 1H), 7.76-7.67 (m, 3H), 7.66-7.57 (m, 2H), 7.53-7.46 (m, |
| | 2H), 7.46-7.40 (m, 1H), 7.40-7.34 (m, 1H), 7.30 (s, 2H), 7.21-7.11 (m, 2H), 6.24 (s, 1H), |
| | 5.94-5.84 (m, 1H), 4.57-4.45 (m, 2H), 4.42-4.31 (m, 1H), 4.14-3.96 (m, 4H), 3.21-3.04 |
| | (m, 4H), 3.02-2.91 (m, 2H), 2.77-2.63 (m, 3H), 2.59-2.54 (m, 1H), 2.36-2.29 (m, 2H), |
| | 2.28-2.19 (m, 1H), 2.14-2.03 (m, 2H), 2.03-1.93 (m, 1H), 1.90-1.82 (m, 1H), 1.80-1.59 |
| | (m, 7H), 1.21-1.08 (m, 1H), 1.08-0.96 (m, 1H). |
| TDH-408 | ¹H NMR (400 MHz, DMSO) δ 11.13 (s, 1H), 10.64 (s, 1H), 8.70 (s, 1H), 8.34-8.28 (m, |
| | 1H), 7.98-7.88 (m, 2H), 7.76-7.70 (m, 2H), 7.66-7.57 (m, 2H), 7.54-7.40 (m, 4H), 7.34- |
| | 7.26 (m, 2H), 7.26-7.21 (m, 1H), 7.19-7.11 (m, 3H), 6.24 (s, 1H), 5.91-5.82 (m, 1H), |
| | 4.56-4.44 (m, 2H), 4.43-4.33 (m, 1H), 4.22-4.15 (m, 2H), 4.03-3.92 (m, 1H), 3.25-3.14 |
| | (m, 2H), 3.04-2.90 (m, 2H), 2.82-2.73 (m, 2H), 2.70-2.60 (m, 2H), 2.27-2.19 (m, 1H), |
| | 2.17-2.05 (m, 2H), 1.97-1.80 (m, 2H), 1.79-1.62 (m, 4H), 1.47-1.37 (m, 1H). |
| TDH-409 | ¹H NMR (400 MHz, DMSO) δ 11.14 (s, 1H), 10.64 (s, 1H), 8.70 (s, 1H), 8.33-8.28 (m, |
| | 1H), 8.05-7.97 (m, 1H), 7.94 (s, 1H), 7.76-7.70 (m, 2H), 7.66-7.57 (m, 2H), 7.53-7.40 |
| | (m, 3H), 7.35-7.25 (m, 2H), 7.20-7.10 (m, 3H), 6.95-6.88 (m, 1H), 6.24 (s, 1H), 5.93- |
| | 5.83 (m, 1H), 4.58-4.47 (m, 2H), 4.44-4.36 (m, 1H), 4.34-4.26 (m, 2H), 4.23-4.13 (m, |
| | 3H), 4.00-3.91 (m, 1H), 3.71-3.62 (m, 1H), 3.18-3.09 (m, 1H), 3.05-2.93 (m, 2H), 2.82- |
| | 2.60 (m, 5H), 2.28-2.20 (m, 1H), 2.18-2.05 (m, 2H), 1.95-1.84 (m, 1H), 1.79-1.68 (m, |
| | 3H), 1.62-1.53 (m, 1H), 1.49-.38 (m, 1H). |
| TDH-410 | ¹H NMR (400 MHz, DMSO) δ 11.15 (s, 1H), 10.64 (s, 1H), 8.70 (s, 1H), 8.31 (s, 1H), |
| | 8.05-7.98 (m, 1H), 7.94 (s, 1H), 7.78-7.68 (m, 3H), 7.66-7.55 (m, 2H), 7.53-7.46 (m, |
| | 2H), 7.46-7.40 (m, 1H), 7.40-7.36 (m, 1H), 7.34-7.26 (m, 2H), 7.21-7.12 (m, 2H), 6.24 |
| | (s, 1H), 5.94-5.84 (m, 1H), 4.59-4.45 (m, 2H), 4.45-4.34 (m, 1H), 4.21-4.01 (m, 4H), |
| | 3.18-3.05 (m, 3H), 3.05-2.91 (m, 3H), 2.80-2.63 (m, 4H), 2.28-2.18 (m, 1H), 2.17-2.02 |
| | (m, 2H), 1.91-1.83 (m, 1H), 1.80-1.48 (m, 9H), 1.42-1.31 (m, 1H). |
| TDH-411 | ¹H NMR (400 MHz, DMSO) δ 11.18 (s, 1H), 10.63 (s, 1H), 8.85 (s, 1H), 8.70 (s, 1H), 8.30 |
| | (s, 1H), 7.94 (s, 1H), 7.84-7.77 (m, 1H), 7.76-7.69 (m, 2H), 7.66-7.58 (m, 2H), 7.52-7.46 |
| | (m, 2H), 7.46-7.40 (m, 1H), 7.30 (s, 2H), 7.28-7.22 (m, 1H), 7.20-7.12 (m, 2H), 7.04- |
| | 6.98 (m, 1H), 6.24 (s, 1H), 5.93-5.85 (m, 1H), 4.57-4.45 (m, 2H), 4.42-4.34 (m, 1H), |
| | 4.18-4.08 (m, 2H), 4.08-3.98 (m, 1H), 3.97-3.87 (m, 1H), 3.25-3.16 (m, 1H), 3.12-3.01 |
| | (m, 1H), 2.97-2.91 (m, 1H), 2.78-2.66 (m, 4H), 2.40-2.30 (m, 2H), 2.29-2.20 (m, 1H), |
| | 2.14-2.05 (m, 2H), 2.02-1.93 (m, 1H), 1.90-1.83 (m, 1H), 1.81-1.69 (m, 3H), 1.23-1.16 |
| | (m, 1H), 1.12-1.00 (m, 1H). |
| TDH-412 | ¹H NMR (400 MHz, DMSO) δ 11.14 (s, 1H), 10.64 (s, 1H), 8.70 (s, 1H), 8.29 (s, 1H), |
| | 7.98-7.88 (m, 2H), 7.77-7.68 (m, 2H), 7.67-7.57 (m, 3H), 7.52-7.46 (m, 2H), 7.46-7.40 |
| | (m, 1H), 7.39-7.33 (m, 1H), 7.33-7.26 (m, 2H), 7.20-7.11 (m, 2H), 6.24 (s, 1H), 5.96- |
| | 5.84 (m, 1H), 4.58-4.44 (m, 2H), 4.44-4.34 (m, 1H), 4.08-3.94 (m, 2H), 3.49-3.38 (m, |
| | 2H), 3.25-3.14 (m, 1H), 3.11-2.91 (m, 2H), 2.88-2.71 (m, 4H), 2.70-2.62 (m, 2H), 2.62- |
| | 2.54 (m, 1H), 2.37-2.29 (m, 2H), 2.30-2.19 (m, 1H), 2.14-2.04 (m, 2H), 1.95-1.83 (m, |
| | 3H), 1.83-1.58 (m, 6H), 1.19-1.08 (m, 1H), 1.08-0.95 (m, 1H). |
| TDH-413 | ¹H NMR (400 MHz, DMSO) δ 11.18 (s, 1H), 10.64 (s, 1H), 8.90-8.80 (m, 1H), 8.70 (s, |
| | 1H), 8.31 (s, 1H), 7.95 (s, 1H), 7.86-7.77 (m, 1H), 7.77-7.67 (m, 2H), 7.66-7.57 (m, 2H), |
| | 7.53-7.47 (m, 2H), 7.47-7.40 (m, 1H), 7.34-7.28 (m, 2H), 7.28-7.23 (m, 1H), 7.20-7.12 |
| | (m, 2H), 7.04-6.96 (m, 1H), 6.24 (s, 1H), 5.95-5.82 (m, 1H), 4.57-4.46 (m, 2H), 4.46- |
| | 4.35 (m, 1H), 4.22-4.07 (m, 3H), 4.00-3.90 (m, 1H), 3.28-3.21 (m, 1H), 3.21-3.11 (m, |
| | 1H), 3.07-2.91 (m, 2H), 2.86-2.61 (m, 5H), 2.29-2.21 (m, 1H), 2.17-2.04 (m, 2H), 1.94- |
| | 1.83 (m, 1H), 1.77-1.70 (m, 2H), 1.67-1.56 (m, 1H), 1.49-1.38 (m, 1H). |
| TDH-414 | ¹H NMR (400 MHz, DMSO) δ 11.15 (s, 1H), 10.64 (s, 1H), 8.70 (s, 1H), 8.31 (s, 1H), |
| | 7.98-7.88 (m, 2H), 7.77-7.69 (m, 2H), 7.67-7.58 (m, 3H), 7.53-7.39 (m, 3H), 7.39-7.33 |
| | (m, 1H), 7.33-7.24 (m, 2H), 7.20-7.11 (m, 2H), 6.24 (s, 1H), 5.96-5.86 (m, 1H), 4.58- |
| | 4.39 (m, 3H), 4.21-4.08 (m, 1H), 4.08-3.98 (m, 1H), 3.49-3.39 (m, 2H), 3.30-3.22 (m, |
| | 1H), 3.16-3.05 (m, 1H), 3.05-2.91 (m, 2H), 2.89-2.72 (m, 4H), 2.72-2.61 (m, 3H), 2.30- |
| | 2.20 (m, 1H), 2.17-2.04 (m, 2H), 1.93-1.81 (m, 3H), 1.77-1.64 (m, 5H), 1.59-1.49 (m, |
| | 1H), 1.43-1.32 (m, 1H). |
| TDH-415 | ¹H NMR (400 MHz, DMSO-d6) δ 11.10 (s, 1H), 10.62 (s, 1H), 8.69 (s, 1H), 8.29 (s, 1H), |
| | 7.93 (s, 1H), 7.79-7.71 (m, 3H), 7.63-7.58 (m, 2H), 7.52-7.47 (m, 2H), 7.44 (dd, J = 7.3, |
| | 2.8 Hz, 2H), 7.33-7.28 (m, 3H), 7.19-7.12 (m, 2H), 6.23 (s, 1H), 5.17 (s, 2H), 5.13-5.07 |
| | (m, 1H), 4.54-4.47 (m, 2H), 3.26-3.13 (m, 2H), 3.05-2.98 (m, 1H), 2.96-2.84 (m, 1H), |
| | 2.82-2.67 (m, 2H), 2.61-2.58 (m, 2H), 2.43-2.37 (m, 2H), 2.13-2.01 (m, 2H), 1.78-1.69 |
| | (m, 4H), 1.54-1.42 (m, 4H), 1.25-1.16 (m, 2H), 1.08-0.91 (m, 2H). |
| TDH-416 | ¹H NMR (400 MHz, DMSO-d6) δ 11.11 (s, 1H), 10.62 (s, 1H), 8.69 (s, 1H), 8.29 (s, 1H), |
| | 7.93 (s, 1H), 7.86-7.81 (m, 1H), 7.76-7.68 (m, 2H), 7.64-7.59 (m, 2H), 7.52-7.46 (m, |
| | 2H), 7.44-7.39 (m, 2H), 7.34-7.27 (m, 3H), 7.18-7.12 (m, 2H), 6.23 (s, 1H), 5.17-5.13 |
| | (m, 1H), 5.12-5.09 (m, 2H), 4.54-4.47 (m, 2H), 3.26-3.11 (m, 2H), 3.06-2.96 (m, 1H), |
| | 2.93-2.84 (m, 1H), 2.80-2.67 (m, 2H), 2.62-2.57 (m, 2H), 2.42-2.38 (m, 2H), 2.08-2.05 |
| | (m, 2H), 1.78-1.71 (m, 4H), 1.56-1.44 (m, 4H), 1.24-1.17 (m, 2H), 0.98 (d, J = 11.8 Hz, 2H). |
| TDH-417 | ¹H NMR (400 MHz, DMSO-d6) δ 11.08 (s, 1H), 10.63 (s, 1H), 8.69 (s, 1H), 8.29 (s, 1H), |
| | 7.93 (s, 1H), 7.74-7.65 (m, 3H), 7.63-7.57 (m, 2H), 7.51-7.46 (m, 2H), 7.45-7.40 (m, |
| | 1H), 7.35-7.32 (m, 2H), 7.30-7.26 (m, 2H), 7.18-7.12 (m, 2H), 6.23 (s, 1H), 5.12-5.06 |
| | (m, 1H), 4.53-4.47 (m, 2H), 3.74-3.68 (m, 2H), 3.19-3.15 (m, 2H), 3.05-2.92 (m, 4H), |
| | 2.88-2.81 (m, 2H), 2.76-2.70 (m, 1H), 2.59-2.52 (m, 2H), 2.41-2.37 (m, 2H), 2.06 (d, J = |
| | 17.2 Hz, 4H), 1.76-1.72 (m, 4H), 1.52-1.43 (m, 4H), 1.28-1.16 (m, 2H), 1.13-1.03 (m, |
| | 2H), 1.00-0.87 (m, 2H). |
| TDH-418 | ¹H NMR (400 MHz, DMSO-d6) δ 11.07 (s, 1H), 10.63 (s, 1H), 8.69 (s, 1H), 8.29 (s, 1H), |
| | 7.93 (s, 1H), 7.75-7.70 (m, 2H), 7.67-7.64 (m, 1H), 7.62-7.58 (m, 2H), 7.51-7.47 (m, |
| | 2H), 7.44-7.41 (m, 1H), 7.32-7.29 (m, 2H), 7.28-7.21 (m, 2H), 7.19-7.13 (m, 2H), 6.23 |
| | (s, 1H), 5.10-5.03 (m, 1H), 4.51-4.46 (m, 2H), 4.04-4.01 (m, 2H), 3.20-3.16 (m, 2H), |
| | 3.11-3.04 (m, 2H), 3.01-2.94 (m, 2H), 2.90-2.83 (m, 1H), 2.79-2.69 (m, 2H), 2.58-2.53 |
| | (m, 2H), 2.42-2.38 (m, 2H), 2.11-2.03 (m, 4H), 1.73-1.65 (m, 4H), 1.54-1.44 (m, 4H), |
| | 1.30-1.16 (m, 2H), 1.12-1.00 (m, 2H), 0.98-0.86 (m, 2H). |
| TDH-419 | ¹H NMR (400 MHz, DMSO) δ 11.08 (s, 1H), 10.63 (s, 1H), 8.69 (s, 1H), 8.30 (s, 1H), 7.93 |
| | (s, 1H), 7.73-7.68 (m, 2H), 7.68-7.64 (m, 1H), 7.62-7.59 (m, 2H), 7.51-7.47 (m, 2H), |
| | 7.45-7.39 (m, 1H), 7.34-7.26 (m, 4H), 7.18-7.12 (m, 2H), 6.23 (s, 1H), 5.11-5.03 (m, |
| | 1H), 4.53-4.43 (m, 2H), 4.05-3.97 (m, 1H), 3.24-3.16 (m, 1H), 2.88-2.80 (m, 2H), 2.77- |
| | 2.69 (m, 2H), 2.57-2.54 (m, 2H), 2.44-2.39 (m, 2H), 2.12-1.96 (m, 4H), 1.85-1.71 (m, |
| | 4H), 1.57-1.43 (m, 4H), 1.39-1.33 (m, 2H). |
| TDH-420 | ¹H NMR (400 MHz, DMSO) δ 11.07 (s, 1H), 10.63 (s, 1H), 8.69 (s, 1H), 8.29 (s, 1H), 7.93 |
| | (s, 1H), 7.74-7.70 (m, J = 7.2 Hz, 2H), 7.65-7.59 (m, 3H), 7.52-7.47 (m, J = 7.0 Hz, 2H), |
| | 7.44-7.40 (m, J = 7.3 Hz, 1H), 7.31-7.29 (m, J = 3.7 Hz, 2H), 7.28-7.21 (m, 2H), 7.18- |
| | 7.12 (m, J = 8.9 Hz, 2H), 6.23 (s, 1H), 5.10-5.02 (m, J = 12.9, 5.4 Hz, 1H), 4.52-4.45 (m, |
| | J = 11.4 Hz, 2H), 4.05-4.03 (m, 1H), 3.20-3.15 (m, J = 5.1 Hz, 1H), 2.97-2.92 (m, J = |
| | 12.0 Hz, 2H), 2.76-2.68 (m, J = 10.8 Hz, 2H), 2.61-2.56 (m, J = 2.6 Hz, 2H), 2.42-2.39 |
| | (m, 2H), 2.03-1.92 (m, J = 14.3, 6.5 Hz, 4H), 1.84-1.75 (m, J = 12.2 Hz, 4H), 1.55-1.44 |
| | (m, 4H), 1.23-1.19 (m, 2H). |
| TDH-421 | ¹H NMR (400 MHz, DMSO) δ 11.10 (s, 1H), 10.62 (s, 1H), 8.69 (s, 1H), 8.29 (s, 1H), 7.93 |
| | (s, 1H), 7.75-7.69 (m, J = 7.2 Hz, 2H), 7.64-7.57 (m, J = 9.1, 5.1 Hz, 3H), 7.52-7.46 (m, J = |
| | 7.3 Hz, 2H), 7.45-7.40 (m, J = 7.2 Hz, 1H), 7.30 (s, 2H), 7.21-7.08 (m, J = 18.0, 8.9 |
| | Hz, 4H), 7.06 (1, J = 7.0 Hz, 1H), 6.23 (s, 1H), 5.10-5.03 (m, J = 12.9, 5.3 Hz, 1H), 4.52- |
| | 4.44 (m, 2H), 4.17 (s, 2H), 4.04-3.97 (m, J = 12.8 Hz, 1H), 3.25-3.14 (m, J = 19.0, 8.8 |
| | Hz, 2H), 3.04-2.95 (m, J = 12.3 Hz, 1H), 2.93-2.83 (m, J = 22.5, 9.1 Hz, 1H), 2.78-2.70 |
| | (m, J = 11.7 Hz, 1H), 2.66-2.54 (m, J = 20.8, 6.9 Hz, 3H), 2.43-2.34 (m, J = 7.3 Hz, 2H), |
| | 2.12-1.96 (m, J = 15.9 Hz, 4H), 1.91-1.83 (m, J = 11.1 Hz, 1H), 1.79-1.70 (m, J = 7.7 Hz, |
| | 3H), 1.56-1.43 (m, 3H), 1.23-1.09 (m, 2H). |
| TDH-422 | ¹H NMR (400 MHz, DMSO-d6) δ 11.06 (s, 1H), 10.63 (s, 1H), 8.69 (s, 1H), 8.29 (s, 1H), |
| | 7.93 (s, 1H), 7.75-7.70 (m, 2H), 7.63-7.55 (m, 3H), 7.52-7.46 (m, 2H), 7.45-7.39 (m, |
| | 1H), 7.30 (s, 2H), 7.20-7.12 (m, 3H), 7.09-7.00 (m, 2H), 6.23 (s, 1H), 5.08-5.00 (m, 1H), |
| | 4.53-4.43 (m, 2H), 4.15-4.09 (m, 2H), 4.05-4.00 (m, 1H), 3.25-3.15 (m, 2H), 3.05-2.94 |
| | (m, 1H), 2.92-2.82 (m, 1H), 2.78-2.70 (m, 1H), 2.64-2.54 (m, 3H), 2.43-2.36 (m, 2H), |
| | 2.14-1.95 (m, 4H), 1.93-1.83 (m, 1H), 1.80-1.69 (m, 3H), 1.58-1.43 (m, 3H), 1.25-1.11 |
| | (m, 2H). |
| TDH-423 | ¹H NMR (400 MHz, DMSO) δ 10.63 (s, 1H), 8.69 (s, 1H), 8.28 (s, 1H), 7.93 (s, 1H), 7.76- |
| | 7.69 (m, 3H), 7.65-7.58 (m, 3H), 7.54-7.45 (m, 3H), 7.45-7.40 (m, 1H), 7.34-7.23 (m, |
| | 3H), 7.20-7.11 (m, 2H), 6.24 (s, 1H), 5.20-5.16 (m, 1H), 4.52-4.45 (m, 2H), 4.03-3.94 |
| | (m, 1H), 3.24-3.12 (m, 2H), 2.81-2.66 (m, 2H), 2.63-2.55 (m, 1H), 2.39-2.28 (m, 4H), |
| | 2.11-2.03 (m, 4H), 1.78-1.67 (m, 2H), 1.63-1.56 (m, 2H), 1.53-1.47 (m, 5H). |
| TDH-424 | ¹H NMR (300 MHz, DMSO) δ 9.11 (s, 1H), 8.90 (s, 1H), 8.71 (s, 1H), 8.37-8.28 (m, 1H), |
| | 8.29-8.20 (m, 2H), 7.99 (dd, J = 2.3, 0.7 Hz, 1H), 7.71-7.59 (m, 2H), 7.51-7.33 (m, 4H), |
| | 6.25 (s, 0.5H), 6.09 (s, 0.5H), 4.62-4.48 (m, 1H), 3.44-3.36 (m, 2H), 3.16-3.00 (m, 2H), |
| | 2.28-2.14 (m, 4H), 1.91-1.75 (m, 1H), 1.72-1.57 (m, 2H), 1.53-1.41 (m, 2H), 1.41-1.18 |
| | (m, 3H), 1.15-1.01 (m, 1H), 1.00-0.88 (m, 1H), 0.85 (d, J = 6.3 Hz, 3H). |
| TDH-425 | ¹H NMR (400 MHz, DMSO) δ 11.12 (s, 1H), 10.63 (s, 1H), 8.69 (s, 1H), 8.30 (s, 1H), 7.92 |
| | (s, 1H), 7.87-7.81 (m, 1H), 7.76-7.70 (m, 2H), 7.65-7.58 (m, 2H), 7.53-7.46 (m, 2H), |
| | 7.46-7.39 (m, 2H), 7.36-7.25 (m, 3H), 7.20-7.12 (m, 2H), 6.24 (s, 1H), 5.17-5.08 (m, |
| | 3H), 4.34-4.23 (m, 1H), 4.23-4.13 (m, 1H), 3.85-3.73 (m, 1H), 3.09-2.99 (m, 1H), 2.96- |
| | 2.80 (m, 5H), 2.67-2.55 (m, 3H), 2.22-2.10 (m, 4H), 2.09-1.93 (m, 9H), 1.78-1.66 (m, |
| | 5H), 1.58-1.41 (m, 2H), 1.17-1.06 (m, 3H). |
| TDH-426 | ¹H NMR (400 MHz, DMSO) δ 11.07 (s, 1H), 10.62 (s, 1H), 8.69 (s, 1H), 8.30 (s, 1H), 7.92 |
| | (s, 1H), 7.76-7.68 (m, 2H), 7.65-7.54 (m, 3H), 7.53-7.46 (m, 2H), 7.45-7.40 (m, 1H), |
| | 7.29 (s, 2H), 7.19-7.10 (m, 3H), 7.09-7.05 (m, 1H), 7.04-6.98 (m, 1H), 6.24 (s, 1H), |
| | 5.08-5.00 (m, 1H), 4.40-4.32 (m, 1H), 4.21-4.14 (m, 1H), 4.12-4.07 (m, 1H), 3.97-3.89 |
| | (m, 1H), 3.07-2.99 (m, 1H), 2.95-2.81 (m, 5H), 2.69-2.55 (m, 4H), 2.21-2.11 (m, 4H), |
| | 2.11-1.92 (m, 9H), 1.78-1.67 (m, 5H), 1.57-1.43 (m, 2H), 1.18-1.07 (m, 3H). |
| TDH-427 | ¹H NMR (400 MHz, DMSO-d6) δ 11.08 (s, 1H), 10.63 (s, 1H), 8.69 (s, 1H), 8.29 (s, 1H), |
| | 7.93 (s, 1H), 7.76-7.69 (m, 2H), 7.63-7.58 (m, 2H), 7.53-7.46 (m, 2H), 7.45-7.39 (m, |
| | 1H), 7.32-7.27 (m, 2H), 7.17-7.11 (m, 2H), 7.04-7.00 (m, 1H), 6.76-6.66 (m, 2H), 6.23 |
| | (s, 1H), 5.08-5.02 (m, 1H), 4.51-4.45 (m, 2H), 3.56-3.42 (m, 4H), 3.20-3.11 (m, 3H), |
| | 2.96-2.92 (m, 1H), 2.90-2.80 (m, 3H), 2.73-2.67 (m, 1H), 2.62-2.54 (m, 2H), 2.40-2.32 |
| | (m, 2H), 2.12-2.04 (m, 2H), 2.03 (s, 1H), 1.69-1.51 (m, 4H), 1.50-1.33 (m, 4H), 1.32- |
| | 1.25 (m, 2H). |
| TDH-428 | ¹H NMR (400 MHz, DMSO-d6) δ 11.07 (s, 1H), 10.63 (s, 1H), 8.69 (s, 1H), 8.29 (s, 1H), |
| | 7.93 (s, 1H), 7.75-7.70 (m, 2H), 7.66-7.63 (m, 1H), 7.63-7.58 (m, 2H), 7.51-7.46 (m, |
| | 2H), 7.45-7.40 (m, 1H), 7.33-7.25 (m, 2H), 7.20-7.11 (m, 2H), 6.85-6.81 (m, 1H), 6.73- |
| | 6.65 (m, 1H), 6.23 (s, 1H), 5.09-5.01 (m, 1H), 4.54-4.45 (m, 2H), 4.26-4.19 (m, 2H), |
| | 4.15-4.08 (m, 2H), 4.04-3.98 (m, 1H), 3.28-3.10 (m, 3H), 3.02-2.90 (m, 2H), 2.87-2.82 |
| | (m, 1H), 2.71-2.65 (m, 1H), 2.63-2.54 (m, 3H), 2.43-2.37 (m, 2H), 2.10-2.02 (m, 2H), |
| | 1.81-1.62 (m, 4H), 1.59-1.41 (m, 4H), 1.17-1.02 (m, 2H). |
| TDH-429 | ¹H NMR (400 MHz, DMSO) δ 11.08 (s, 1H), 10.63 (s, 1H), 8.69 (s, 1H), 8.29 (s, 1H), 7.93 |
| | (s, 1H), 7.76-7.70 (m, 2H), 7.70-7.57 (m, 3H), 7.51-7.46 (m, 2H), 7.44-7.39 (m, 1H), |
| | 7.38-7.22 (m, 4H), 7.19-7.10 (m, 2H), 6.24 (s, 1H), 5.14-5.03 (m, 1H), 4.48 (s, 2H), 3.67 |
| | (s, 2H), 3.23-3.14 (m, 2H), 3.01-2.82 (m, 4H), 2.79-2.65 (m, 1H), 2.63-2.54 (m, 2H), |
| | 2.39 (s, 2H), 2.33-2.26 (m, 2H), 2.15-1.95 (m, 4H), 1.93-1.82 (m, 2H), 1.80-1.65 (m, |
| | 6H), 1.56-1.43 (m, 4H), 1.43-1.35 (m, 2H), 1.09-0.88 (m, 2H). |
| TDH-430 | ¹H NMR (400 MHz, DMSO) δ 11.08 (s, 1H), 10.63 (s, 1H), 8.69 (s, 1H), 8.29 (s, 1H), 7.93 |
| | (s, 1H), 7.74-7.71 (m, 2H), 7.67-7.57 (m, 3H), 7.51-7.47 (m, 2H), 7.46-7.38 (m, 1H), |
| | 7.33-7.27 (m, 3H), 7.24-7.19 (m, 1H), 7.16-7.11 (m, 2H), 6.24 (s, 1H), 5.10-5.02 (m, |
| | 1H), 4.54-4.46 (m, 2H), 4.06-4.00 (m, 2H), 3.24-3.15 (m, 2H), 3.01-2.81 (m, 4H), 2.80- |
| | 2.65 (m, 1H), 2.64-2.55 (m, 2H), 2.42-2.36 (m, 2H), 2.27-2.22 (m, 2H), 2.14-1.94 (m, |
| | 5H), 1.91-1.84 (m, 1H), 1.80-1.64 (m, 6H), 1.57-1.39 (m, 4H), 1.27-1.17 (m, 2H), 1.05- |
| | 0.90 (m, 2H). |
| TDH-431 | ¹H NMR (400 MHz, DMSO-d6) δ 11.12 (s, 1H), 10.63 (s, 1H), 8.69 (s, 1H), 8.29 (s, 1H), |
| | 7.93 (s, 1H), 7.79-7.74 (m, 3H), 7.74-7.69 (m, 2H), 7.65-7.57 (m, 2H), 7.52-7.46 (m, |
| | 2H), 7.44-7.39 (m, 1H), 7.34-7.26 (m, 2H), 7.18-7.10 (m, 2H), 6.23 (s, 1H), 5.17-5.09 |
| | (m, 1H), 4.52-4.45 (m, 2H), 3.28-3.21 (m, 3H), 3.20-3.14 (m, 1H), 3.00-2.82 (m, 3H), |
| | 2.79-2.60 (m, 5H), 2.60-2.54 (m, 1H), 2.42-2.32 (m, 2H), 2.13-2.01 (m, 3H), 1.82-1.64 |
| | (m, 4H), 1.54-1.37 (m, 4H), 1.04-0.88 (m, 2H). |
| TDH-432 | ¹H NMR (400 MHz, DMSO-d6) δ 11.12 (s, 1H), 10.63 (s, 1H), 8.69 (s, 1H), 8.29 (s, 1H), |
| | 7.93 (s, 1H), 7.85-7.80 (m, 2H), 7.77-7.70 (m, 3H), 7.63-7.58 (m, 2H), 7.52-7.46 (m, |
| | 2H), 7.46-7.40 (m, 1H), 7.34-7.25 (m, 2H), 7.20-7.11 (m, 2H), 6.23 (s, 1H), 5.19-5.09 |
| | (m, 1H), 4.55-4.45 (m, 2H), 3.24-3.13 (m, 2H), 3.05-2.97 (m, 2H), 2.96-2.83 (m, 3H), |
| | 2.79-2.65 (m, 4H), 2.64-2.53 (m, 2H), 2.40-2.32 (m, 2H), 2.11-2.02 (m, 3H), 1.78-1.64 |
| | (m, 4H), 1.55-1.38 (m, 4H), 0.99-0.86 (m, 2H). |
| TDH-433 | ¹H NMR (400 MHz, DMSO) δ 11.17 (s, 1H), 10.63 (s, 1H), 8.84 (s, 1H), 8.69 (s, 1H), 8.29 |
| | (s, 1H), 7.93 (s, 1H), 7.85-7.77 (m, 1H), 7.77-7.69 (m, 2H), 7.63-7.57 (m, 2H), 7.51-7.45 |
| | (m, 2H), 7.45-7.39 (m, 1H), 7.30 (s, 2H), 7.27-7.20 (m, 1H), 7.20-7.11 (m, 2H), 7.04- |
| | 6.97 (m, 1H), 6.23 (s, 1H), 5.92-5.83 (m, 1H), 4.53-4.43 (m, 2H), 4.43-4.36 (m, 1H), |
| | 4.14-4.06 (m, 2H), 3.96-3.89 (m, 1H), 3.23-3.14 (m, 1H), 3.03-2.88 (m, 2H), 2.78-2.58 |
| | (m, 4H), 2.44-2.35 (m, 2H), 2.27-2.20 (m, 1H), 2.13-2.02 (m, 2H), 1.83-1.66 (m, 4H), |
| | 1.58-1.39 (m, 4H), 1.19-0.98 (m, 2H). |
| TDH-434 | ¹H NMR (400 MHz, DMSO) δ 11.12 (s, 1H), 10.63 (s, 1H), 8.69 (s, 1H), 8.29 (s, 1H), 7.93 |
| | (s, 1H), 7.92-7.88 (m, 1H), 7.73-7.69 (m, 2H), 7.64-7.57 (m, 2H), 7.52-7.46 (m, 3H), |
| | 7.46-7.40 (m, 1H), 7.30 (s, 2H), 7.25-7.20 (m, 1H), 7.20-7.12 (m, 3H), 6.23 (s, 1H), |
| | 5.90-5.82 (m, 1H), 4.53-4.42 (m, 2H), 4.40-4.35 (m, 1H), 4.16-4.11 (m, 1H), 4.02-3.92 |
| | (m, 2H), 3.24-3.11 (m, 1H), 3.06-2.87 (m, 2H), 2.76-2.56 (m, 4H), 2.41-2.37 (m, 2H), |
| | 2.25-2.17 (m, 1H), 2.11-2.01 (m, 2H), 1.88-1.65 (m, 4H), 1.61-1.34 (m, 4H), 1.23-0.98 |
| | (m, 2H). |
| TDH-435 | ¹H NMR (400 MHz, DMSO) δ 11.14 (s, 1H), 10.63 (s, 1H), 8.69 (s, 1H), 8.30 (s, 1H), 7.94 |
| | (s, 1H), 7.92-7.89 (m, 1H), 7.75-7.70 (m, 2H), 7.65-7.58 (m, 3H), 7.52-7.47 (m, 2H), |
| | 7.46-7.40 (m, 1H), 7.38-7.33 (m, 1H), 7.33-7.29 (m, 2H), 7.19-7.12 (m, 2H), 6.24 (s, |
| | 1H), 5.95-5.86 (m, 1H), 4.55-4.48 (m, 2H), 3.47-3.39 (m, 2H), 3.27-3.13 (m, 3H), 3.03- |
| | 2.93 (m, 2H), 2.90-2.78 (m, 3H), 2.78-2.67 (m, 3H), 2.43-2.36 (m, 2H), 2.28-2.22 (m, |
| | 1H), 2.14-2.05 (m, 3H), 1.95-1.86 (m, 2H), 1.80-1.64 (m, 6H), 1.55-1.41 (m, 4H), 1.11- |
| | 0.97 (m, 2H). |
| TDH-436 | ¹H NMR (400 MHz, DMSO) δ 11.14 (s, 1H), 10.63 (s, 1H), 8.69 (s, 1H), 8.29 (s, 1H), |
| | 8.03-7.98 (m, 1H), 7.93 (s, 1H), 7.74-7.72 (m, 2H), 7.72-7.69 (m, 1H), 7.63-7.58 (m, |
| | 2H), 7.52-7.46 (m, 2H), 7.46-7.40 (m, 1H), 7.39-7.35 (m, 1H), 7.30 (s, 2H), 7.19-7.12 |
| | (m, 2H), 6.24 (s, 1H), 5.91-5.84 (m, 1H), 4.48 (s, 2H), 4.09 (m, 2H), 3.27-3.06 (m, 4H), |
| | 3.04-2.91 (m, 4H), 2.71-2.60 (m, 4H), 2.43-2.36 (m, 2H), 2.25-2.19 (m, 1H), 2.14-2.01 |
| | (m, 2H), 1.93-1.82 (m, 2H), 1.79-1.68 (m, 6H), 1.53-1.43 (m, 4H), 1.09-0.93 (m, 2H). |
| TDH-437 | ¹H NMR (400 MHz, DMSO-d6) δ 11.18 (s, 1H), 10.63 (s, 1H), 8.69 (s, 1H), 8.29 (s, 1H), |
| | 8.23-8.16 (m, 1H), 7.93 (s, 1H), 7.76-7.65 (m, 3H), 7.65-7.57 (m, 2H), 7.57-7.52 (m, |
| | 1H), 7.53-7.45 (m, 2H), 7.47-7.38 (m, 1H), 7.30 (s, 2H), 7.20-7.10 (m, 2H), 6.23 (s, 1H), |
| | 5.99-5.90 (m, 1H), 5.24-5.11 (m, 2H), 4.53-4.44 (m, 2H), 3.25-3.14 (m, 1H), 3.09-2.87 |
| | (m, 3H), 2.79-2.54 (m, 5H), 2.44-2.34 (m, 2H), 2.30-2.23 (m, 1H), 2.12-2.00 (m, 2H), |
| | 1.81-1.66 (m, 4H), 1.59-1.42 (m, 4H), 1.26-1.03 (m, 2H). |
| TDH-438 | ¹H NMR (400 MHz, DMSO-d6) δ 11.14 (s, 1H), 10.62 (s, 1H), 8.69 (s, 1H), 8.29 (s, 1H), |
| | 8.04-7.96 (m, 1H), 7.93 (s, 1H), 7.76-7.69 (m, 2H), 7.65-7.57 (m, 2H), 7.54-7.45 (m, |
| | 2H), 7.47-7.38 (m, 1H), 7.30 (s, 2H), 7.20-7.10 (m, 3H), 6.93-6.86 (m, 1H), 6.23 (s, 1H), |
| | 5.93-5.84 (m, 1H), 4.55-4.44 (m, 2H), 4.33-4.24 (m, 2H), 4.21-4.13 (m, 2H), 3.66-3.57 |
| | (m, 1H), 3.24-3.17 (m, 1H), 3.07-2.86 (m, 3H), 2.80-2.54 (m, 5H), 2.43-2.34 (m, 2H), |
| | 2.27-2.18 (m, 1H), 2.14-2.03 (m, 2H), 1.85-1.66 (m, 4H), 1.59-1.39 (m, 4H), 1.18-0.99 |
| | (m, 2H). |
| TDH-439 | ¹H NMR (400 MHz, DMSO-d6) δ 10.99 (s, 1H), 10.63 (s, 1H), 8.69 (s, 1H), 8.29 (s, 1H), |
| | 7.93 (s, 1H), 7.75-7.67 (m, 2H), 7.65-7.58 (m, 2H), 7.58-7.54 (m, 1H), 7.53-7.48 (m, |
| | 2H), 7.48-7.45 (m, 1H), 7.45-7.39 (m, 2H), 7.30 (s, 2H), 7.19-7.10 (m, 2H), 6.23 (s, 1H), |
| | 5.16-5.08 (m, 1H), 4.52-4.45 (m, 2H), 4.41-4.31 (m, 2H), 3.24-3.14 (m, 2H), 2.98-2.83 |
| | (m, 4H), 2.78-2.53 (m, 6H), 2.48-2.43 (m, 1H), 2.38-2.31 (m, 2H), 2.11-1.97 (m, 3H), |
| | 1.90-1.62 (m, 4H), 1.54-1.28 (m, 4H), 0.97-0.82 (m, 2H). |
| TDH-440 | ¹H NMR (400 MHz, DMSO) δ 11.12 (s, 1H), 10.68 (s, 1H), 8.70 (s, 1H), 8.29 (s, 1H), 7.93 |
| | (s, 1H), 7.88-7.80 (m, 1H), 7.78-7.70 (m, 2H), 7.66-7.59 (m, 2H), 7.54-7.46 (m, 2H), |
| | 7.46-7.39 (m, 2H), 7.36-7.26 (m, 3H), 7.19-7.09 (m, 2H), 6.25 (s, 1H), 5.18-5.04 (m, |
| | 3H), 4.29-4.18 (m, 2H), 3.84-3.73 (m, 1H), 3.10-2.96 (m, 3H), 2.96-2.84 (m, 1H), 2.67- |
| | 2.56 (m, 4H), 2.44-2.30 (m, 9H), 2.21-2.10 (m, 4H), 2.10-1.89 (m, 8H), 1.79-1.62 (m, 6H). |
| TDH-441 | ¹H NMR (400 MHz, DMSO) δ 11.07 (s, 1H), 10.66 (s, 1H), 8.70 (s, 1H), 8.29 (s, 1H), 7.93 |
| | (s, 1H), 7.78-7.69 (m, 2H), 7.67-7.55 (m, 3H), 7.53-7.39 (m, 3H), 7.30 (s, 2H), 7.21-7.11 |
| | (m, 3H), 7.11-7.05 (m, 1H), 7.05-6.97 (m, 1H), 6.25 (s, 1H), 5.11-4.99 (m, 1H), 4.43- |
| | 4.32 (m, 1H), 4.31-4.15 (m, 2H), 4.15-4.04 (m, 2H), 3.97-3.86 (m, 1H), 3.05-2.95 (m, |
| | 3H), 2.95-2.81 (m, 2H), 2.70-2.57 (m, 4H), 2.44-2.24 (m, 9H), 2.19-2.08 (m, 4H), 2.08- |
| | 1.92 (m, 6H), 1.89-1.69 (m, 4H), 1.69-1.55 (m, 2H). |
| TDH-442 | ¹H NMR (400 MHz, DMSO) δ 11.12 (s, 1H), 10.66 (s, 1H), 8.69 (s, 1H), 8.29 (s, 1H), 7.92 |
| | (s, 1H), 7.87-7.81 (m, 1H), 7.77-7.70 (m, 2H), 7.65-7.58 (m, 2H), 7.52-7.46 (m, 2H), |
| | 7.45-7.40 (m, 2H), 7.35-7.32 (m, 1H), 7.30 (s, 2H), 7.19-7.11 (m, 2H), 6.25 (s, 1H), |
| | 5.17-5.09 (m, 3H), 4.33-4.22 (m, 2H), 4.22-4.12 (m, 1H), 3.82-3.75 (m, 1H), 3.08-2.98 |
| | (m, 2H), 2.94-2.88 (m, 4H), 2.67-2.55 (m, 6H), 2.12-2.01 (m, 5H), 2.01-1.92 (m, 6H), |
| | 1.78-1.67 (m, 4H), 1.58-1.48 (m, 6H). |
| TDH-443 | ¹H NMR (400 MHz, DMSO) δ 11.07 (s, 1H), 10.63 (s, 1H), 8.69 (s, 1H), 8.29 (s, 1H), 7.92 |
| | (s, 1H), 7.77-7.68 (m, 2H), 7.65-7.55 (m, 3H), 7.54-7.47 (m, 2H), 7.47-7.39 (m, 1H), |
| | 7.30 (s, 2H), 7.18-7.10 (m, 3H), 7.10-7.05 (m, 1H), 7.05-6.98 (m, 1H), 6.24 (s, 1H), |
| | 5.09-5.00 (m, 1H), 4.42-4.32 (m, 1H), 4.27-4.14 (m, 2H), 4.13-4.06 (m, 2H), 3.99-3.89 |
| | (m, 1H), 3.08-2.96 (m, 2H), 2.93-2.84 (m, 3H), 2.72-2.55 (m, 6H), 2.09-1.90 (m, 12H), |
| | 1.79-1.66 (m, 4H), 1.60-1.49 (m, 6H). |
| TDH-444 | ¹H NMR (500 MHz, DMSO) δ 10.70 (s, 1H), 9.18 (s, 1H), 9.03-8.94 (m, 1H), 8.77-8.73 |
| | (m, 1H), 8.72 (s, 1H), 8.25 (s, 1H), 7.99 (s, 1H), 7.66-7.62 (m, 2H), 7.50-7.44 (m, 3H), |
| | 7.42-7.39 (m, 1H), 6.08 (s, 1H), 4.59-4.54 (m, 1H), 3.79-3.71 (m, 1H), 3.44-3.34 (m, |
| | 4H), 3.11-3.05 (m, 2H), 3.04-2.94 (m, 2H), 2.70-2.65 (m, 3H), 2.26-2.18 (m, 4H), 1.92- |
| | 1.81 (m, 2H), 1.81-1.71 (m, 2H). |
| TDH-445 | ¹H NMR (400 MHz, DMSO) δ 10.62 (s, 1H), 10.34 (s, 1H), 8.69 (s, 1H), 8.30 (s, 1H), 7.92 |
| | (s, 1H), 7.75-7.70 (m, 2H), 7.64-7.59 (m, 2H), 7.52-7.47 (m, 2H), 7.46-7.40 (m, 1H), |
| | 7.39-7.36 (m, 1H), 7.34-7.32 (m, 1H), 7.31-7.27 (m, 2H), 7.19-7.12 (m, 3H), 6.24 (s, |
| | 1H), 4.27-4.13 (m, 2H), 3.85 (s, 3H), 3.61 (t, J = 6.6 Hz, 2H), 2.98-2.93 (m, 2H), 2.71- |
| | 2.67 (m, 2H), 2.24-2.20 (m, 2H), 2.14-2.08 (m, 2H), 2.03-1.94 (m, 5H), 1.86-1.67 (m, |
| | 5H), 1.12-1.05 (m, 2H). |
| TDH-446 | ¹H NMR (400 MHz, DMSO) δ 10.62 (s, 1H), 10.34 (s, 1H), 8.70 (s, 1H), 8.29 (s, 1H), 7.92 |
| | (s, 1H), 7.76-7.70 (m, 2H), 7.66-7.58 (m, 2H), 7.52-7.46 (m, 2H), 7.46-7.40 (m, 1H), |
| | 7.40-7.35 (m, 1H), 7.35-7.27 (m, 3H), 7.19-7.12 (m, 3H), 6.24 (s, 1H), 4.26-4.13 (m, |
| | 2H), 3.85 (s, 3H), 3.60 (t, J = 6.7 Hz, 2H), 3.01-2.95 (m, 2H), 2.73-2.66 (m, 2H), 2.42- |
| | 2.34 (m, 2H), 2.12-1.90 (m, 7H), 1.77-1.65 (m, 2H), 1.64-1.54 (m, 1H), 1.47-1.39 (m, |
| | 2H), 1.18-1.07 (m, 2H). |
| TDH-447 | ¹H NMR (400 MHz, DMSO) δ 10.62 (s, 1H), 10.34 (s, 1H), 8.69 (s, 1H), 8.29 (s, 1H), 7.92 |
| | (s, 1H), 7.75-7.70 (m, 2H), 7.64-7.58 (m, 2H), 7.52-7.46 (m, 2H), 7.46-7.38 (m, 2H), |
| | 7.36-7.34 (m, 1H), 7.29 (s, 2H), 7.19-7.12 (m, 3H), 6.24 (s, 1H), 4.27-4.14 (m, 2H), 3.85 |
| | (s, 3H), 3.60 (t, J = 6.7 Hz, 2H), 3.00-2.93 (m, 2H), 2.71-2.67 (m, 2H), 2.40-2.33 (m, 7H), |
| | 2.09-1.92 (m, 8H), 1.65-1.59 (m, 2H), 0.87-0.83 (m, 2H). |
| TDH-448 | ¹H NMR (400 MHz, DMSO) δ 10.62 (s, 1H), 10.34 (s, 1H), 8.69 (s, 1H), 8.29 (s, 1H), 7.92 |
| | (s, 1H), 7.76-7.70 (m, 2H), 7.65-7.59 (m, 2H), 7.53-7.46 (m, 2H), 7.46-7.41 (m, 1H), |
| | 7.41-7.37 (m, 1H), 7.37-7.34 (m, 1H), 7.29 (s, 2H), 7.19-7.12 (m, 3H), 6.24 (s, 1H), |
| | 4.22-4.13 (m, 1H), 3.84 (s, 3H), 3.63-3.53 (m, 6H), 3.29-3.21 (m, 2H), 3.03-2.95 (m, |
| | 2H), 2.72-2.65 (m, 2H), 2.59-2.54 (m, 2H), 2.23-2.14 (m, 2H), 2.06-1.90 (m, 5H), 1.90- |
| | 1.80 (m, 2H), 1.51-1.42 (m, 2H). |
| TDH-450 | ¹H NMR (500 MHz, DMSO) δ 10.64 (s, 1H), 8.70 (s, 1H), 8.30 (s, 1H), 7.92 (s, 1H), 7.76- |
| | 7.71 (m, 2H), 7.68-7.59 (m, 3H), 7.53-7.47 (m, 2H), 7.46-7.42 (m, 1H), 7.34-7.28 (m, |
| | 3H), 7.26-7.22 (m, 1H), 7.20-7.13 (m, 2H), 6.24 (s, 1H), 5.18-5.10 (m, 1H), 4.44-4.38 |
| | (m, 1H), 4.23-4.15 (m, 1H), 4.08-4.01 (m, 2H), 3.93-3.88 (m, 1H), 3.02 (s, 3H), 3.00- |
| | 2.91 (m, 6H), 2.80-2.73 (m, 1H), 2.60-2.52 (m, 3H), 2.29-2.25 (m, 2H), 2.20-2.16 (m, |
| | 2H), 2.07-1.95 (m, 8H), 1.79-1.74 (m, 4H), 1.27-1.21 (m, 2H), 1.05-0.98 (m, 1H), 0.94- |
| | 0.85 (m, 1H). |
| TDH-451 | ¹H NMR (400 MHz, DMSO) δ 11.12 (s, 1H), 10.62 (s, 1H), 8.69 (s, 1H), 8.29 (s, 1H), 7.98 |
| | (s, 1H), 7.92 (s, 1H), 7.87-7.81 (m, 1H), 7.77-7.68 (m, 2H), 7.65-7.57 (m, 2H), 7.54-7.39 |
| | (m, 4H), 7.39-7.34 (m, 1H), 7.29 (s, 2H), 7.19-7.10 (m, 2H), 6.23 (s, 1H), 5.18 (s, 2H), |
| | 5.16-5.09 (m, 1H), 4.82-4.72 (m, 1H), 4.48-4.35 (m, 1H), 4.28-4.14 (m, 1H), 3.99-3.86 |
| | (m, 1H), 3.08-2.98 (m, 2H), 2.95-2.75 (m, 4H), 2.68-2.55 (m, 4H), 2.21-1.89 (m, 11H), |
| | 1.89-1.71 (m, 1H). |
| TDH-452 | ¹H NMR (400 MHz, DMSO) δ 11.07 (s, 1H), 10.62 (s, 1H), 8.69 (s, 1H), 8.29 (s, 1H), 7.98 |
| | (s, 1H), 7.92 (s, 1H), 7.76-7.69 (m, 2H), 7.65-7.55 (m, 3H), 7.52-7.46 (m, 2H), 7.46-7.39 |
| | (m, 1H), 7.29 (s, 2H), 7.19-7.12 (m, 3H), 7.13-7.07 (m, 1H), 7.07-7.01 (m, 1H), 6.23 (s, |
| | 1H), 5.08-5.01 (m, 1H), 4.82-4.72 (m, 1H), 4.53-4.42 (m, 1H), 4.25-4.13 (m, 3H), 4.13- |
| | 4.02 (m, 1H), 3.07-2.98 (m, 2H), 2.94-2.78 (m, 4H), 2.69-2.58 (m, 3H), 2.58-2.54 (m, |
| | 1H), 2.23-2.08 (m, 4H), 2.08-1.89 (m, 7H), 1.89-1.73 (m, 1H). |
| TDH-453 | ¹H NMR (400 MHz, DMSO) δ 11.12 (s, 1H), 10.65 (s, 1H), 8.69 (s, 1H), 8.29 (s, 1H), 7.92 |
| | (s, 1H), 7.88-7.80 (m, 1H), 7.77-7.69 (m, 2H), 7.66-7.59 (m, 2H), 7.52-7.46 (m, 2H), |
| | 7.46-7.40 (m, 2H), 7.39-7.33 (m, 1H), 7.29 (s, 2H), 7.20-7.11 (m, 2H), 6.25 (s, 1H), |
| | 5.18-5.10 (m, 1H), 5.04-4.89 (m, 1H), 4.56-4.49 (m, 1H), 4.26-4.16 (m, 1H), 3.69-3.62 |
| | (m, 2H), 3.21-3.16 (m, 2H), 3.03-2.96 (m, 2H), 2.91-2.84 (m, 2H), 2.64-2.57 (m, 3H), |
| | 2.45-2.35 (m, 2H), 2.19-1.91 (m, 8H), 1.90-1.68 (m, 2H), 1.65-1.52 (m, 3H). |
| TDH-454 | ¹H NMR (400 MHz, DMSO) δ 11.06 (s, 1H), 10.63 (s, 1H), 8.69 (s, 1H), 8.29 (s, 1H), 7.92 |
| | (s, 1H), 7.76-7.69 (m, 2H), 7.65-7.56 (m, 3H), 7.53-7.46 (m, 2H), 7.46-7.39 (m, 1H), |
| | 7.29 (s, 2H), 7.18-7.13 (m, 2H), 7.14-7.04 (m, 2H), 7.03-6.95 (m, 1H), 6.24 (s, 1H), |
| | 5.10-5.01 (m, 1H), 4.59-4.53 (m, 1H), 4.24-4.12 (m, 2H), 4.03-3.95 (m, 2H), 3.71-3.59 |
| | (m, 2H), 3.02-2.93 (m, 2H), 2.89-2.82 (m, 2H), 2.61-2.55 (m, 3H), 2.41-2.32 (m, 2H), |
| | 2.10-1.92 (m, 8H), 1.86-1.69 (m, 2H), 1.64-1.51 (m, 3H). |
| TDH-455 | ¹H NMR (300 MHz, MeOD) δ 8.59 (s, 1H), 8.25-8.21 (m, 1H), 8.06-7.99 (m, 1H), 7.70- |
| | 7.62 (m, 2H), 7.58-7.48 (m, 3H), 6.79 (s, 1H), 4.65-4.56 (m, 1H), 3.68 (s, 3H), 3.63-3.53 |
| | (m, 4H), 3.29-3.14 (m, 4H), 3.03-2.78 (m, 4H), 2.39-2.26 (m, 4H). |
| TDH-456 | ¹H NMR (300 MHz, MeOD) δ 8.60 (s, 1H), 8.55 (s, 0.5H), 8.36 (s, 0.5H), 8.33 (s, 1H), |
| | 8.11-8.09 (m, 1H), 7.78-7.74 (m, 2H), 7.74-7.72 (m, 1H), 7.54-7.42 (m, 4H), 7.37-7.26 |
| | (m, 1H), 7.18-7.11 (m, 1H), 6.38 (s, 1H), 4.68-4.61 (m, 1H), 3.62-3.57 (m, 2H), 3.27- |
| | 3.21 (m, 2H), 2.39-2.34 (m, 4H). |
| TDH-457 | ¹H NMR (300 MHz, DMSO) δ 9.42-9.32 (m, 1H), 9.21-9.11 (m, 1H), 9.09-9.02 (m, 1H), |
| | 8.72 (s, 1H), 8.26 (s, 1H), 7.98 (s, 1H), 7.70-7.63 (m, 2H), 7.51-7.38 (m, 3H), 7.26-7.18 |
| | (m, 2H), 7.15-7.05 (m, 2H), 6.18 (s, 1H), 4.58-4.53 (m, 1H), 4.29 (t, J = 6.4 Hz, 2H), |
| | 3.44-3.33 (m, 2H), 3.16-2.97 (m, 2H), 2.28-2.14 (m, 4H). |
| TDH-458 | ¹H NMR (400 MHz, DMSO) δ 10.38 (s, 1H), 9.10-9.00 (m, 1H), 8.86-8.77 (m, 1H), 8.73 |
| | (s, 1H), 8.26 (s, 1H), 8.00 (s, 1H), 7.87-7.81 (m, 1H), 7.78-7.71 (m, 2H), 7.54-7.47 (m, |
| | 2H), 7.47-7.41 (m, 1H), 7.31-7.24 (m, 1H), 7.23-7.13 (m, 2H), 6.48 (s, 1H), 4.60-4.51 |
| | (m, 1H), 3.44-3.37 (m, 2H), 3.14-3.03 (m, 2H), 2.28-2.12 (m, 4H). |
| TDH-459 | ¹H NMR (400 MHz, DMSO) δ 11.11 (s, 1H), 9.04-8.96 (m, 1H), 8.82-8.75 (m, 1H), 8.73 |
| | (s, 1H), 8.25 (s, 1H), 8.00 (s, 1H), 7.87-7.81 (m, 2H), 7.79-7.73 (m, 2H), 7.73-7.66 (m, |
| | 2H), 7.55-7.48 (m, 2H), 7.48-7.41 (m, 1H), 6.32 (s, 1H), 4.60-4.51 (m, 1H), 3.43-3.36 |
| | (m, 2H), 3.14-3.02 (m, 2H), 2.28-2.11 (m, 4H). |
| TDH-460 | ¹H NMR (400 MHz, DMSO) δ 11.12 (s, 1H), 9.06-8.97 (m, 1H), 8.84-8.76 (m, 1H), 8.74 |
| | (s, 1H), 8.26 (s, 1H), 8.13-8.10 (m, 1H), 8.00 (s, 1H), 7.87-7.81 (m, 1H), 7.80-7.73 (m, |
| | 2H), 7.61-7.54 (m, 1H), 7.54-7.47 (m, 2H), 7.47-7.40 (m, 2H), 6.31 (s, 1H), 4.60-4.51 |
| | (m, 1H), 3.44-3.38 (m, 2H), 3.15-3.04 (m, 2H), 2.27-2.14 (m, 4H). |
| TDH-461 | ¹H NMR (500 MHz, DMSO) δ 10.92 (s, 1H), 10.62 (s, 1H), 9.27-9.19 (m, 1H), 9.05-8.94 |
| | (m, 1H), 8.73 (s, 1H), 8.26 (s, 1H), 8.00 (s, 1H), 7.78-7.73 (m, 2H), 7.54-7.48 (m, 4H), |
| | 7.46-7.40 (m, 1H), 7.00-6.95 (m, 2H), 6.29 (s, 1H), 4.61-4.52 (m, 1H), 3.75-3.71 (m, |
| | 2H), 3.48-3.44 (m, 2H), 3.43-3.36 (m, 2H), 3.15-3.02 (m, 6H), 2.81-2.77 (m, 3H), 2.26- |
| | 2.16 (m, 4H). |
| TDH-462 | ¹H NMR (500 MHz, DMSO) δ 10.79 (s, 1H), 10.70 (s, 1H), 9.19-9.11 (m, 1H), 9.00-8.91 |
| | (m, 1H), 8.74 (s, 1H), 8.27 (s, 1H), 8.00 (s, 1H), 7.80-7.73 (m, 2H), 7.54-7.46 (m, 2H), |
| | 7.46-7.39 (m, 1H), 7.40-7.36 (m, 1H), 7.23-7.16 (m, 1H), 7.12-7.06 (m, 1H), 6.79-6.71 |
| | (m, 1H), 6.30 (s, 1H), 4.63-4.51 (m, 1H), 3.51-3.46 (m, 2H), 3.43-3.37 (m, 2H), 3.14- |
| | 3.03 (m, 6H), 2.80 (d, J = 4.6 Hz, 3H), 2.25-2.13 (m, 4H). |
| TDH-463 | ¹H NMR (400 MHz, DMSO) δ 10.73 (s, 1H), 9.24-9.13 (m, 1H), 9.02-8.90 (m, 1H), 8.73 |
| | (s, 1H), 8.26 (s, 1H), 8.00 (s, 1H), 7.81-7.72 (m, 2H), 7.65-7.58 (m, 2H), 7.53-7.48 (m, |
| | 2H), 7.47-7.37 (m, 2H), 7.36-7.24 (m, 3H), 7.13-7.03 (m, 1H), 6.31 (s, 1H), 4.62-4.52 |
| | (m, 1H), 3.44-3.37 (m, 2H), 3.13-3.02 (m, 2H), 2.26-2.15 (m, 4H). |
| TDH-464 | ¹H NMR (400 MHz, DMSO) δ 10.73 (s, 1H), 9.22-9.13 (m, 1H), 9.01-8.89 (m, 1H), 8.73 |
| | (s, 1H), 8.26 (s, 1H), 7.99 (s, 1H), 7.78-7.72 (m, 2H), 7.57-7.45 (m, 3H), 7.46-7.38 (m, |
| | 2H), 7.12-7.04 (m, 1H), 6.29 (s, 1H), 4.63-4.51 (m, 1H), 3.46-3.36 (m, 2H), 3.15-3.01 |
| | (m, 2H), 2.30-2.14 (m, 7H). |
| TDH-465 | ¹H NMR (400 MHz, DMSO) δ 10.65 (s, 1H), 9.17-9.08 (m, 1H), 8.92-8.81 (m, 1H), 8.72 |
| | (s, 1H), 8.19 (s, 1H), 7.95 (s, 1H), 7.69-7.58 (m, 4H), 7.52-7.45 (m, 2H), 7.23-7.15 (m, |
| | 2H), 6.64 (s, 1H), 4.60-4.49 (m, 1H), 3.41-3.36 (m, 2H), 3.10-3.00 (m, 2H), 2.22-2.13 |
| | (m, 4H). |
| TDH-466 | ¹H NMR (400 MHz, DMSO) δ 11.11 (s, 1H), 10.64 (s, 1H), 8.69 (s, 1H), 8.28 (s, 1H), 7.92 |
| | (s, 1H), 7.85-7.81 (m, 1H), 7.76-7.69 (m, 2H), 7.65-7.59 (m, 2H), 7.53-7.46 (m, 2H), |
| | 7.46-7.39 (m, 2H), 7.38-7.33 (m, 1H), 7.29 (s, 2H), 7.19-7.11 (m, 2H), 6.24 (s, 1H), |
| | 5.16-5.07 (m, 1H), 5.02 (s, 2H), 4.23-4.16 (m, 1H), 3.76-3.69 (m, 2H), 3.63-3.58 (m, |
| | 2H), 3.02-2.94 (m, 2H), 2.92-2.84 (m, 2H), 2.81-2.70 (m, 2H), 2.64-2.55 (m, 4H), 2.46- |
| | 2.33 (m, 4H), 2.17-1.93 (m, 8H), 1.68-1.59 (m, 2H). |
| TDH-467 | ¹H NMR (400 MHz, DMSO) δ 11.06 (s, 1H), 10.66 (s, 1H), 8.69 (s, 1H), 8.28 (s, 1H), 7.92 |
| | (s, 1H), 7.75-7.71 (m, 2H), 7.66-7.55 (m, 3H), 7.52-7.46 (m, 2H), 7.46-7.39 (m, 1H), |
| | 7.29 (s, 2H), 7.20-7.08 (m, 4H), 7.02-6.96 (m, 1H), 6.25 (s, 1H), 5.08-4.99 (m, 1H), |
| | 4.22-4.15 (m, 1H), 4.08-4.02 (m, 2H), 3.78-3.70 (m, 2H), 3.62-3.58 (m, 2H), 3.00-2.93 |
| | (m, 2H), 2.91-2.83 (m, 2H), 2.83-2.72 (m, 2H), 2.63-2.54 (m, 4H), 2.45-2.33 (m, 4H), |
| | 2.14-1.89 (m, 8H), 1.69-1.56 (m, 2H). |
| TDH-468 | ¹H NMR (500 MHz, DMSO) δ 12.18 (s, 1H), 9.26-9.16 (m, 2H), 9.05-8.96 (m, 1H), 8.75 |
| | (s, 1H), 8.66-8.59 (m, 2H), 8.27 (s, 1H), 8.02-7.94 (m, 2H), 7.87-7.82 (m, 2H), 7.54-7.49 |
| | (m, 2H), 7.49-7.43 (m, 1H), 6.43 (s, 1H), 4.60-4.53 (m, 1H), 3.42-3.37 (m, 2H), 3.12- |
| | 3.05 (m, 2H), 2.25-2.16 (m, 4H). |
| TDH-469 | ¹H NMR (400 MHz, DMSO) δ 10.95 (s, 1H), 9.26-9.15 (m, 1H), 9.06-8.95 (m, 1H), 8.73 |
| | (s, 1H), 8.26 (s, 1H), 8.00 (s, 1H), 7.81-7.74 (m, 2H), 7.68 (s, 1H), 7.55-7.48 (m, 2H), |
| | 7.48-7.30 (m, 4H), 6.33 (s, 1H), 4.61-4.52 (m, 1H), 3.43-3.36 (m, 2H), 3.14-3.05 (m, |
| | 2H), 3.00 (s, 6H), 2.28-2.17 (m, 4H). |
| TDH-470 | ¹H NMR (400 MHz, DMSO) δ 11.03 (s, 1H), 9.22-9.11 (m, 1H), 9.03-8.87 (m, 1H), 8.73 |
| | (s, 1H), 8.26 (s, 1H), 7.99 (s, 1H), 7.79-7.69 (m, 4H), 7.62 (s, 2H), 7.54-7.46 (m, 2H), |
| | 7.46-7.36 (m, 1H), 6.32 (s, 1H), 4.63-4.51 (m, 1H), 3.43-3.36 (m, 2H), 3.14-3.07 (m, |
| | 2H), 3.06 (s, 6H), 2.26-2.16 (m, 4H). |
| TDH-471 | ¹H NMR (400 MHz, DMSO) δ 10.47 (s, 1H), 9.30-9.18 (m, 1H), 9.06-8.92 (m, 1H), 8.71 |
| | (s, 1H), 8.26 (s, 1H), 8.00 (s, 1H), 7.70-7.60 (m, 2H), 7.24-7.11 (m, 2H), 5.06 (d, J = 5.0 |
| | Hz, 1H), 4.62-4.54 (m, 1H), 3.44-3.36 (m, 2H), 3.16-3.04 (m, 2H), 2.40-2.31 (m, 1H), |
| | 2.28-2.15 (m, 4H), 1.09 (d, J = 6.7 Hz, 6H). |
| TDH-472 | ¹H NMR (400 MHz, DMSO) δ 11.07 (s, 1H), 10.61 (s, 1H), 8.69 (s, 1H), 8.29 (s, 1H), 7.91 |
| | (s, 1H), 7.75-7.70 (m, 2H), 7.67-7.58 (m, 3H), 7.52-7.47 (m, 2H), 7.47-7.41 (m, 1H), |
| | 7.31-7.27 (m, 2H), 7.27-7.20 (m, 2H), 7.20-7.12 (m, 2H), 6.24 (s, 1H), 5.11-5.03 (m, |
| | 1H), 4.22-4.15 (m, 1H), 4.07-4.00 (m, 2H), 3.00-2.83 (m, 6H), 2.74-2.65 (m, 2H), 2.60- |
| | 2.55 (m, 2H), 2.35-2.26 (m, 2H), 2.25-2.17 (m, 2H), 2.13-2.07 (m, 2H), 2.05-2.01 (m, |
| | 2H), 1.99-1.86 (m, 8H), 1.80-1.76 (m, 2H), 1.56-1.46 (m, 6H). |
| TDH-473 | ¹H NMR (400 MHz, DMSO) δ 11.07 (s, 1H), 10.63 (s, 1H), 8.69 (s, 1H), 8.29 (s, 1H), 7.92 |
| | (s, 1H), 7.76-7.71 (m, 2H), 7.68-7.59 (m, 3H), 7.53-7.46 (m, 2H), 7.46-7.41 (m, 1H), |
| | 7.33-7.26 (m, 3H), 7.25-7.20 (m, 1H), 7.20-7.12 (m, 2H), 6.24 (s, 1H), 5.10-5.03 (m, |
| | 1H), 4.22-4.13 (m, 1H), 4.08-4.01 (m, 2H), 3.02-2.80 (m, 8H), 2.64-2.53 (m, 3H), 2.21- |
| | 2.16 (m, 2H), 2.06-1.93 (m, 8H), 1.82-1.76 (m, 3H), 1.72-1.66 (m, 2H), 1.22-1.11 (m, 6H). |
| TDH-474 | ¹H NMR (400 MHz, DMSO) δ 11.07 (s, 1H), 10.62 (s, 1H), 8.69 (s, 1H), 8.29 (s, 1H), 7.92 |
| | (s, 1H), 7.76-7.71 (m, 2H), 7.67-7.57 (m, 3H), 7.51-7.46 (m, 2H), 7.46-7.41 (m, 1H), |
| | 7.33-7.26 (m, 3H), 7.24-7.20 (m, 1H), 7.20-7.11 (m, 2H), 6.24 (s, 1H), 5.10-5.02 (m, |
| | 1H), 4.21-4.14 (m, 1H), 4.06-3.99 (m, 2H), 3.01-2.91 (m, 4H), 2.91-2.83 (m, 1H), 2.63- |
| | 2.59 (m, 1H), 2.59-2.54 (m, 2H), 2.41-2.27 (m, 10H), 2.15-2.09 (m, 2H), 2.09-1.89 (m, |
| | 8H), 1.84-1.74 (m, 3H), 1.65-1.53 (m, 2H), 1.18-1.06 (m, 2H). |
| TDH-475 | ¹H NMR (400 MHz, DMSO) δ 10.98 (s, 1H), 10.62 (s, 1H), 8.69 (s, 1H), 8.28 (s, 1H), 7.92 |
| | (s, 1H), 7.77-7.69 (m, 2H), 7.67-7.56 (m, 3H), 7.53-7.45 (m, 3H), 7.45-7.36 (m, 2H), |
| | 7.28 (s, 2H), 7.20-7.10 (m, 2H), 6.24 (s, 1H), 5.15-5.05 (m, 1H), 4.47-4.24 (m, 3H), |
| | 4.22-4.11 (m, 1H), 3.87-3.78 (m, 1H), 3.01-2.85 (m, 6H), 2.70-2.62 (m, 2H), 2.46-2.28 |
| | (m, 3H), 2.13-1.88 (m, 8H), 1.71-1.60 (m, 2H), 1.58-1.41 (m, 2H), 1.40-1.31 (m, 2H), |
| | 0.96-0.84 (m, 2H). |
| TDH-476 | ¹H NMR (400 MHz, DMSO) δ 10.98 (s, 1H), 10.63 (s, 1H), 8.69 (s, 1H), 8.28 (s, 1H), 7.92 |
| | (s, 1H), 7.76-7.70 (m, 2H), 7.66-7.58 (m, 3H), 7.51-7.46 (m, 3H), 7.46-7.36 (m, 2H), |
| | 7.28 (s, 2H), 7.20-7.11 (m, 2H), 6.24 (s, 1H), 5.14-5.07 (m, 1H), 4.42 (d, J = 17.3 Hz, |
| | 1H), 4.29 (d, J = 17.2 Hz, 1H), 4.24-4.14 (m, 1H), 3.00-2.88 (m, 5H), 2.72-2.56 (m, 4H), |
| | 2.45-2.22 (m, 10H), 2.11-1.89 (m, 9H), 1.66-1.56 (m, 2H). |
| TDH-477 | ¹H NMR (400 MHz, DMSO) δ 10.63 (s, 1H), 10.33 (s, 1H), 8.69 (s, 1H), 8.29 (s, 1H), 7.92 |
| | (s, 1H), 7.76-7.70 (m, 2H), 7.65-7.58 (m, 2H), 7.52-7.46 (m, 2H), 7.46-7.40 (m, 1H), |
| | 7.39-7.34 (m, 1H), 7.34-7.31 (m, 1H), 7.29 (s, 2H), 7.20-7.12 (m, 3H), 6.24 (s, 1H), |
| | 4.23-4.14 (m, 1H), 3.85 (s, 3H), 3.63-3.57 (m, 2H), 2.98-2.80 (m, 6H), 2.72-2.66 (m, |
| | 2H), 2.22-2.11 (m, 4H), 2.11-1.89 (m, 10H), 1.76-1.67 (m, 4H), 1.57-1.43 (m, 2H), 1.14- |
| | 1.03 (m, 4H). |
| TDH-478 | ¹H NMR (400 MHz, DMSO) δ 10.63 (s, 1H), 10.33 (s, 1H), 8.69 (s, 1H), 8.29 (s, 1H), 7.92 |
| | (s, 1H), 7.75-7.70 (m, 2H), 7.64-7.58 (m, 2H), 7.52-7.47 (m, 2H), 7.45-7.40 (m, 1H), |
| | 7.38-7.34 (m, 1H), 7.33-7.26 (m, 3H), 7.20-7.12 (m, 3H), 6.24 (s, 1H), 4.25-4.15 (m, |
| | 2H), 3.85 (s, 3H), 3.62-3.59 (m, 2H), 3.02-2.96 (m, 2H), 2.71-2.67 (m, 2H), 2.43-2.30 |
| | (m, 10H), 2.19-1.90 (m, 12H), 1.84-1.66 (m, 4H), 1.65-1.58 (m, 2H), 1.11-1.00 (m, 2H). |
| TDH-479 | ¹H NMR (400 MHz, DMSO) δ 10.47 (s, 1H), 9.30-9.18 (m, 1H), 9.06-8.92 (m, 1H), 8.71 |
| | (s, 1H), 8.26 (s, 1H), 8.00 (s, 1H), 7.70-7.60 (m, 2H), 7.24-7.11 (m, 2H), 5.06 (d, J = 5.0 |
| | Hz, 1H), 4.62-4.54 (m, 1H), 3.44-3.36 (m, 2H), 3.16-3.04 (m, 2H), 2.40-2.31 (m, 1H), |
| | 2.28-2.15 (m, 4H), 1.09 (d, J = 6.7 Hz, 6H). |
| TDH-480 | ¹H NMR (400 MHz, DMSO) δ 11.11 (s, 1H), 10.64 (s, 1H), 8.69 (s, 1H), 8.28 (s, 1H), 8.09 |
| | (s, 0.5H), 7.92 (s, 1H), 7.85-7.83 (m, 1H), 7.82 (s, 0.5H), 7.78-7.71 (m, 3H), 7.65-7.58 |
| | (m, 2H), 7.53-7.46 (m, 2H), 7.46-7.41 (m, 1H), 7.29 (s, 2H), 7.21-7.12 (m, 2H), 6.25 (s, |
| | 1H), 5.17-5.10 (m, 1H), 4.27-4.18 (m, 2H), 3.64-3.57 (m, 2H), 3.53-3.48 (m, 2H), 3.03- |
| | 2.99 (m, 2H), 2.90-2.82 (m, 2H), 2.68-2.56 (m, 6H), 2.10-2.01 (m, 4H), 2.00-1.94 (m, |
| | 4H), 1.40-1.37 (m, 2H), 1.31-1.26 (m, 3H), 0.88-0.82 (m, 4H). |
| TDH-481 | ¹H NMR (400 MHz, DMSO) δ 11.07 (s, 1H), 10.61 (s, 1H), 8.69 (s, 1H), 8.29 (s, 1H), |
| | 7.93-7.89 (m, 1H), 7.75-7.69 (m, 2H), 7.66-7.57 (m, 3H), 7.52-7.46 (m, 2H), 7.46-7.39 |
| | (m, 1H), 7.32-7.26 (m, 3H), 7.25-7.21 (m, 1H), 7.18-7.12 (m, 2H), 6.23 (s, 1H), 5.08- |
| | 5.02 (m, 1H), 4.20-4.12 (m, 1H), 4.07-3.99 (m, 2H), 3.00-2.83 (m, 6H), 2.63-2.53 (m, |
| | 2H), 2.36-2.26 (m, 2H), 2.04-1.94 (m, 5H), 1.80-1.74 (m, 2H), 1.51-1.45 (m, 2H), 1.26- |
| | 1.11 (m, 6H). |
| TDH-482 | ¹H NMR (400 MHz, DMSO) δ 11.11 (s, 1H), 10.62 (s, 1H), 8.69 (s, 1H), 8.29 (s, 1H), 7.92 |
| | (s, 1H), 7.85-7.81 (m, 1H), 7.75-7.70 (m, 2H), 7.65-7.58 (m, 2H), 7.52-7.46 (m, 2H), |
| | 7.45-7.41 (m, 2H), 7.35-7.32 (m, 1H), 7.29 (s, 2H), 7.20-7.13 (m, 2H), 6.24 (s, 1H), |
| | 5.17-5.10 (m, 3H), 4.33-4.29 (m, 1H), 4.22-4.14 (m, 1H), 3.85-3.74 (m, 2H), 3.08-2.83 |
| | (m, 6H), 2.63-2.58 (m, 2H), 2.34-2.27 (m, 2H), 2.06-2.01 (m, 4H), 1.99-1.92 (m, 2H), |
| | 1.75-1.68 (m, 2H), 1.50-1.46 (m, 2H), 1.27-1.22 (m, 2H), 1.19-1.12 (m, 1H), 1.01-0.93 |
| | (m, 1H). |
| TDH-483 | ¹H NMR (400 MHz, DMSO) δ 11.06 (s, 1H), 10.62 (s, 1H), 8.69 (s, 1H), 8.29 (s, 1H), 7.92 |
| | (s, 1H), 7.76-7.71 (m, 2H), 7.65-7.55 (m, 3H), 7.53-7.46 (m, 2H), 7.46-7.40 (m, 1H), |
| | 7.29 (s, 2H), 7.20-7.12 (m, 3H), 7.08-7.00 (m, 2H), 6.24 (s, 1H), 5.08-5.01 (m, 1H), |
| | 4.43-4.36 (m, 1H), 4.23-4.04 (m, 4H), 3.98-3.89 (m, 1H), 3.06-2.83 (m, 5H), 2.68-2.56 |
| | (m, 3H), 2.36-2.27 (m, 2H), 2.06-1.95 (m, 6H), 1.77-1.70 (m, 2H), 1.51-1.47 (m, 2H), |
| | 1.25-1.21 (m, 2H), 1.16-1.08 (m, 1H), 1.02-0.92 (m, 1H). |
| TDH-484 | ¹H NMR (400 MHz, DMSO) δ 11.12 (s, 1H), 10.62 (s, 1H), 8.69 (s, 1H), 8.29 (s, 1H), 7.92 |
| | (s, 1H), 7.85-7.81 (m, 2H), 7.77-7.71 (m, 3H), 7.66-7.58 (m, 2H), 7.52-7.46 (m, 2H), |
| | 7.46-7.40 (m, 1H), 7.29 (s, 2H), 7.21-7.12 (m, 2H), 6.24 (s, 1H), 5.18-5.11 (m, 1H), |
| | 4.41-4.34 (m, 1H), 4.21-4.13 (m, 1H), 3.91-3.83 (m, 1H), 3.03-2.98 (m, 2H), 2.96-2.88 |
| | (m, 4H), 2.76-2.68 (m, 2H), 2.68-2.55 (m, 3H), 2.32-2.25 (m, 2H), 2.10-1.92 (m, 8H), |
| | 1.69-1.61 (m, 2H), 1.47-1.42 (m, 2H), 1.22-1.17 (m, 2H), 0.95-0.85 (m, 2H). |
| TDH-485 | ¹H NMR (400 MHz, DMSO) δ 11.11 (s, 1H), 10.82 (s, 1H), 8.71 (s, 1H), 8.29 (s, 1H), 7.95 |
| | (s, 1H), 7.87-7.82 (m, 1H), 7.82-7.80 (m, 1H), 7.77-7.71 (m, 2H), 7.53-7.42 (m, 5H), |
| | 7.38-7.33 (m, 2H), 7.30 (s, 2H), 7.19-7.10 (m, 1H), 6.26 (s, 1H), 5.19-5.04 (m, 3H), |
| | 4.34-4.23 (m, 1H), 3.49-3.44 (m, 4H), 2.95-2.84 (m, 2H), 2.65-2.54 (m, 4H), 2.48-2.44 |
| | (m, 2H), 2.42-2.34 (m, 4H), 2.09-1.96 (m, 4H), 1.78-1.64 (m, 2H), 1.28-1.22 (m, 4H) |
| TDH-486 | ¹H NMR (400 MHz, DMSO) δ 11.11 (s, 1H), 10.57 (s, 1H), 8.70 (s, 1H), 8.30 (s, 1H), 7.93 |
| | (s, 1H), 7.86-7.81 (m, 1H), 7.76-7.71 (m, 2H), 7.52-7.47 (m, 2H), 7.45-7.41 (m, 2H), |
| | 7.37-7.33 (m, 1H), 7.31-7.27 (m, 3H), 7.24-7.19 (m, 1H), 7.16-7.11 (m, 1H), 6.69-6.62 |
| | (m, 1H), 6.26 (s, 1H), 5.16-5.09 (m, 3H), 4.25-4.16 (m, 1H), 3.72 (s, 3H), 3.49-3.44 (m, |
| | 4H), 3.01-2.84 (m, 4H), 2.63-2.55 (m, 2H), 2.45-2.40 (m, 2H), 2.37-2.34 (m, 4H), 2.08- |
| | 1.98 (m, 6H), 1.67-1.60 (m, 2H), 1.40-1.33 (m, 2H). |
| TDH-487 | ¹H NMR (500 MHz, DMSO) δ 11.12 (s, 1H), 10.49 (s, 1H), 8.71 (s, 1H), 8.30 (s, 1H), 7.95 |
| | (s, 1H), 7.87-7.83 (m, 1H), 7.75-7.71 (m, 2H), 7.52-7.47 (m, 2H), 7.46-7.42 (m, 3H), |
| | 7.38-7.33 (m, 2H), 7.31 (s, 2H), 7.22-7.17 (m, 1H), 6.92-6.88 (m, 1H), 6.26 (s, 1H), |
| | 5.16-5.11 (m, 3H), 4.32-4.19 (m, 1H), 3.49-3.45 (m, 4H), 3.09-2.98 (m, 2H), 2.95-2.83 |
| | (m, 2H), 2.62-2.55 (m, 2H), 2.48-2.43 (m, 2H), 2.41-2.35 (m, 4H), 2.27 (s, 3H), 2.12- |
| | 1.90 (m, 8H), 1.72-1.63 (m, 2H). |
| TDH-488 | ¹H NMR (400 MHz, DMSO) δ 11.14 (s, 1H), 9.26-9.16 (m, 1H), 9.04-8.91 (m, 1H), 8.73 |
| | (s, 1H), 8.26 (s, 1H), 7.99 (s, 1H), 7.84-7.75 (m, 3H), 7.54-7.46 (m, 2H), 7.46-7.34 (m, |
| | 3H), 6.31 (s, 1H), 4.59-4.54 (m, 1H), 3.43-3.37 (m, 2H), 3.12-3.02 (m, 2H), 2.25-2.17 |
| | (m, 4H). |
| TDH-489 | ¹H NMR (400 MHz, DMSO) δ 10.94 (s, 1H), 9.28-9.21 (m, 1H), 9.09-9.01 (m, 1H), 8.73 |
| | (s, 1H), 8.25 (s, 1H), 7.99 (s, 1H), 7.85-7.79 (m, 2H), 7.68-7.61 (m, 2H), 7.38-7.31 (m, |
| | 2H), 7.19-7.12 (m, 2H), 6.33 (s, 1H), 4.58-4.56 (m, 1H), 3.42-3.37 (m, 2H), 3.10-3.04 |
| | (m, 2H), 2.24-2.20 (m, 4H). |
| TDH-490 | ¹H NMR (400 MHz, DMSO) δ 10.56 (s, 0.5H), 10.06 (s, 0.5H), 9.21-9.05 (m, 1H), 8.95- |
| | 8.79 (m, 1H), 8.51-8.45 (m, 1H), 8.19 (s, 1H), 7.81 (s, 0.5H), 7.77 (s, 0.5H), 7.70-7.63 |
| | (m, 1H), 7.57-7.34 (m, 5H), 7.32-7.27 (m, H), 7.22-7.12 (m, 2H), 6.32 (s, 0.5H), 5.79 (s, |
| | 0.5H), 4.57-4.45 (m, 1H), 3.57 (s, 3H), 3.43-3.28 (m, 2H), 3.13-2.92 (m, 2H), 2.25-2.12 |
| | (m, 2H), 2.09-1.88 (m, 2H). |
| TDH-491 | ¹H NMR (400 MHz, DMSO) δ 11.08 (s, 1H), 10.58 (s, 1H), 9.13-9.07 (m, 1H), 8.65 (s, |
| | 1H), 8.37 (s, 1H), 8.01 (s, 1H), 7.71-7.66 (m, 1H), 7.65-7.60 (m, 2H), 7.60-7.55 (m, 2H), |
| | 7.46-7.38 (m, 4H), 7.37-7.32 (m, 2H), 7.29-7.24 (m, 1H), 7.20-7.13 (m, 2H), 5.83-5.79 |
| | (m, 1H), 5.12-5.03 (m, 1H), 4.26-4.17 (m, 1H), 3.49-3.40 (m, 4H), 3.02-2.95 (m, 2H), |
| | 2.91-2.83 (m, 1H), 2.65-2.53 (m, 3H), 2.41-2.31 (m, 5H), 2.15-1.91 (m, 9H), 1.71-1.61 |
| | (m, 2H). |
| TDH-492 | ¹H NMR (400 MHz, DMSO) δ 11.11 (s, 1H), 10.59 (s, 1H), 9.12-9.07 (m, 1H), 8.65 (s, |
| | 1H), 8.37 (s, 1H), 8.01 (s, 1H), 7.87-7.81 (m, 1H), 7.66-7.60 (m, 2H), 7.60-7.56 (m, 2H), |
| | 7.46-7.38 (m, 5H), 7.38-7.32 (m, 2H), 7.21-7.12 (m, 2H), 5.83-5.78 (m, 1H), 5.16-5.09 |
| | (m, 3H), 4.27-4.18 (m, 1H), 3.50-3.43 (m, 4H), 3.06-2.96 (m, 2H), 2.93-2.83 (m, 1H), |
| | 2.63-2.55 (m, 2H), 2.45-2.35 (m, 7H), 2.13-1.93 (m, 8H), 1.68-1.59 (m, 2H). |
| TDH-493 | ¹H NMR (400 MHz, DMSO) δ 11.07 (s, 1H), 10.59 (s, 1H), 9.12-9.06 (m, 1H), 8.65 (s, |
| | 1H), 8.37 (s, 1H), 8.01 (s, 1H), 7.66-7.61 (m, 2H), 7.60-7.57 (m, 3H), 7.46-7.38 (m, 4H), |
| | 7.38-7.32 (m, 1H), 7.20-7.11 (m, 3H), 7.11-7.06 (m, 1H), 7.05-6.99 (m, 1H), 5.83-5.77 |
| | (m, 1H), 5.08-5.01 (m, 1H), 4.27-4.18 (m, 1H), 4.17-4.11 (m, 2H), 3.55-3.48 (m, 4H), |
| | 3.05-2.96 (m, 2H), 2.92-2.83 (m, 1H), 2.62-2.56 (m, 2H), 2.43-2.33 (m, 7H), 2.14-1.95 |
| | (m, 8H), 1.69-1.61 (m, 2H). |
| TDH-494 | ¹H NMR (400 MHz, DMSO) δ 11.12 (s, 1H), 10.59 (s, 1H), 8.69 (s, 1H), 8.24 (s, 1H), 7.88 |
| | (s, 1H), 7.87-7.81 (m, 1H), 7.69-7.58 (m, 4H), 7.52-7.46 (m, 2H), 7.46-7.41 (m, 1H), |
| | 7.37-7.32 (m, 1H), 7.30 (s, 2H), 7.23-7.14 (m, 2H), 6.62 (s, 1H), 5.19-5.09 (m, 3H), |
| | 4.26-4.15 (m, 1H), 3.51-3.42 (m, 4H), 3.06-2.86 (m, 3H), 2.65-2.56 (m, 3H), 2.46-2.39 |
| | (m, 3H), 2.39-2.28 (m, 5H), 2.10-1.93 (m, 6H), 1.71-1.61 (m, 2H). |
| TDH-494 | ¹H NMR (400 MHz, DMSO) δ 11.12 (s, 1H), 10.59 (s, 1H), 8.69 (s, 1H), 8.24 (s, 1H), 7.88 |
| | (s, 1H), 7.87-7.81 (m, 1H), 7.69-7.58 (m, 4H), 7.52-7.46 (m, 2H), 7.46-7.41 (m, 1H), |
| | 7.37-7.32 (m, 1H), 7.30 (s, 2H), 7.23-7.14 (m, 2H), 6.62 (s, 1H), 5.19-5.09 (m, 3H), |
| | 4.26-4.15 (m, 1H), 3.51-3.42 (m, 4H), 3.06-2.86 (m, 3H), 2.65-2.56 (m, 3H), 2.46-2.39 |
| | (m, 3H), 2.39-2.28 (m, 5H), 2.10-1.93 (m, 6H), 1.71-1.61 (m, 2H). |
| TDH-495 | ¹H NMR (400 MHz, DMSO) δ 11.07 (s, 1H), 10.64 (s, 1H), 9.84 (s, 1H), 8.70 (s, 1H), 8.29 |
| | (s, 1H), 7.92 (s, 1H), 7.76-7.73 (m, 1H), 7.72 (s, 1H), 7.65-7.58 (m, 2H), 7.53-7.46 (m, |
| | 2H), 7.46-7.40 (m, 1H), 7.34-7.26 (m, 5H), 7.19-7.12 (m, 2H), 7.01-6.96 (m, 1H), 6.23 |
| | (s, 1H), 5.74 (s, 1H), 5.01-4.93 (m, 1H), 4.23-4.14 (m, 1H), 3.75 (s, 2H), 3.52-3.44 (m, |
| | 4H), 3.01-2.92 (m, 2H), 2.90-2.81 (m, 1H), 2.62-2.54 (m, 2H), 2.48-2.42 (m, 1H), 2.39- |
| | 2.25 (m, 8H), 2.07-1.94 (m, 6H), 1.66-1.56 (m, 2H). |
| TDH-496 | ¹H NMR (400 MHz, DMSO) δ 11.07 (s, 1H), 10.63 (s, 1H), 9.84 (s, 1H), 8.69 (s, 1H), 8.29 |
| | (s, 1H), 7.92 (s, 1H), 7.76-7.70 (m, 2H), 7.65-7.57 (m, 2H), 7.53-7.46 (m, 2H), 7.46-7.40 |
| | (m, 1H), 7.35-7.25 (m, 5H), 7.21-7.12 (m, 2H), 7.02-6.96 (m, 1H), 6.23 (s, 1H), 5.74 (s, |
| | 1H), 5.01-4.92 (m, 1H), 4.22-4.10 (m, 1H), 3.91-3.83 (m, 1H), 3.77-3.67 (m, 3H), 3.58- |
| | 3.46 (m, 3H), 3.27-3.19 (m, 1H), 3.14-3.03 (m, 1H), 3.02-2.94 (m, 2H), 2.91-2.79 (m, |
| | 1H), 2.63-2.52 (m, 2H), 2.48-2.41 (m, 1H), 2.21-2.10 (m, 2H), 2.06-1.85 (m, 6H), 1.83- |
| | 1.68 (m, 2H), 1.34-1.23 (m, 2H). |
| TDH-497 | ¹H NMR (400 MHz, DMSO) δ 10.62 (s, 1H), 10.35 (s, 1H), 8.69 (s, 1H), 8.40-8.32 (m, |
| | 1H), 8.30 (s, 1H), 7.91 (s, 1H), 7.89-7.83 (m, 1H), 7.83-7.77 (m, 1H), 7.77-7.68 (m, 2H), |
| | 7.65-7.59 (m, 2H), 7.52-7.46 (m, 2H), 7.46-7.39 (m, 1H), 7.29 (s, 2H), 7.20-7.12 (m, |
| | 3H), 6.24 (s, 1H), 4.23-4.14 (m, 1H), 3.86 (s, 3H), 3.64-3.56 (m, 2H), 3.15-3.07 (m, 2H), |
| | 2.97-2.89 (m, 2H), 2.74-2.65 (m, 2H), 2.21-2.12 (m, 2H), 2.09-1.93 (m, 6H), 1.85-1.73 |
| | (m, 4H), 1.55-1.41 (m, 2H), 0.96-0.79 (m, 4H). |
| TDH-498 | ¹H NMR (400 MHz, DMSO) δ 11.06 (s, 1H), 10.61 (s, 1H), 9.82 (s, 1H), 8.69 (s, 1H), 8.30 |
| | (s, 1H), 8.08-8.01 (m, 1H), 7.91 (s, 1H), 7.76-7.70 (m, 2H), 7.65-7.59 (m, 2H), 7.53-7.47 |
| | (m, 2H), 7.46-7.41 (m, 1H), 7.37-7.33 (m, 1H), 7.33-7.24 (m, 4H), 7.20-7.11 (m, 2H), |
| | 7.05-6.98 (m, 1H), 6.24 (s, 1H), 5.75 (s, 1H), 5.01-4.92 (m, 1H), 4.20-4.11 (m, 1H), 3.44 |
| | (s, 2H), 2.95-2.86 (m, 4H), 2.61-2.54 (m, 1H), 2.48-2.41 (m, 1H), 2.15-2.05 (m, 2H), |
| | 2.06-1.87 (m, 8H), 1.81-1.73 (m, 2H), 1.73-1.65 (m, 2H), 1.42-1.30 (m, 2H), 0.90-0.76 |
| | (m, 4H). |
| TDH-499 | ¹H NMR (400 MHz, DMSO) δ 10.69 (s, 1H), 10.33 (s, 1H), 8.69 (s, 1H), 8.29 (s, 1H), 7.93 |
| | (s, 1H), 7.77-7.71 (m, 2H), 7.65-7.59 (m, 2H), 7.53-7.46 (m, 2H), 7.46-7.40 (m, 1H), |
| | 7.38-7.35 (m, 1H), 7.33-7.27 (m, 3H), 7.19-7.12 (m, 3H), 6.26 (s, 1H), 4.32-4.17 (m, |
| | 2H), 3.85 (s, 3H), 3.63-3.57 (m, 2H), 3.09-2.88 (m, 3H), 2.73-2.68 (m, 2H), 2.59-2.53 |
| | (m, 2H), 2.13-2.04 (m, 2H), 2.02-1.88 (m, 6H), 1.80-1.66 (m, 3H), 1.66-1.45 (m, 6H), |
| | 1.45-1.35 (m, 1H), 1.33-1.26 (m, 2H), 1.21-1.14 (m, 2H), 1.11-1.05 (m, 2H), 0.94-0.83 |
| | (m, 2H). |
| TDH-500 | ¹H NMR (400 MHz, DMSO) δ 11.06 (s, 1H), 10.62 (s, 1H), 9.82 (s, 1H), 8.69 (s, 1H), 8.29 |
| | (s, 1H), 7.91 (s, 1H), 7.76-7.69 (m, 2H), 7.66-7.59 (m, 2H), 7.53-7.46 (m, 2H), 7.46-7.40 |
| | (m, 1H), 7.34-7.24 (m, 5H), 7.19-7.11 (m, 2H), 7.02-6.97 (m, 1H), 6.24 (s, 1H), 5.72 (s, |
| | 1H), 5.00-4.93 (m, 1H), 4.40-4.34 (m, 1H), 4.26-4.13 (m, 2H), 3.97-3.91 (m, 1H), 3.73 |
| | (s, 2H), 2.93-2.86 (m, 3H), 2.72-2.65 (m, 2H), 2.61-2.57 (m, 2H), 2.25-2.18 (m, 2H), |
| | 2.05-1.98 (m, 4H), 1.95-1.90 (m, 4H), 1.69-1.62 (m, 2H), 1.57-1.50 (m, 4H), 1.48-1.43 |
| | (m, 2H), 1.41-1.34 (m, 2H), 1.33-1.27 (m, 2H), 0.95-0.83 (m, 4H). |
| TDH-501 | ¹H NMR (400 MHz, DMSO) δ 10.65 (s, 1H), 10.34 (s, 1H), 8.69 (s, 1H), 8.29 (s, 1H), 7.92 |
| | (s, 1H), 7.76-7.69 (m, 2H), 7.66-7.58 (m, 2H), 7.52-7.47 (m, 2H), 7.47-7.39 (m, 1H), 7.40- |
| | 7.34 (m, 1H), 7.34-7.25 (m, 3H), 7.21-7.11 (m, 3H), 6.24 (s, 1H), 4.27-4.23 (m, 1H), 4.21- |
| | 4.15 (m, 1H), 3.85 (s, 3H), 3.62-3.58 (m, 2H), 2.95-2.90 (m, 2H), 2.71-2.66 (m, 2H), 2.21- |
| | 2.15 (m, 2H), 2.12-1.89 (m, 10H), 1.76-1.66 (m, 4H), 1.60-1.54 (m, 2H), 1.50-1.38 (m, |
| | 4H), 1.33-1.29 (m, 1H), 1.18-1.07 (m, 4H), 0.89-0.81 (m, 2H). |
| TDH-502 | ¹H NMR (400 MHz, DMSO) δ 10.66 (s, 1H), 10.34 (s, 1H), 8.69 (s, 1H), 8.29 (s, 1H), 7.92 |
| | (s, 1H), 7.77-7.69 (m, 2H), 7.66-7.58 (m, 2H), 7.52-7.46 (m, 2H), 7.46-7.41 (m, 1H), 7.40- |
| | 7.35 (m, 1H), 7.35-7.32 (m, 1H), 7.30 (s, 2H), 7.20-7.11 (m, 3H), 6.25 (s, 1H), 4.40-4.30 |
| | (m, 1H), 4.25-4.14 (m, 1H), 4.03-3.94 (m, 1H), 3.85 (s, 3H), 3.66-3.56 (m, 2H), 2.94-2.90 |
| | (m, 2H), 2.74-2.66 (m, 2H), 2.21-2.15 (m, 2H), 2.09-1.90 (m, 10H), 1.84-1.61 (m, 8H), |
| | 1.58-1.43 (m, 7H), 1.27-1.17 (m, 6H), 1.00-0.89 (m, 2H), 0.89-0.80 (m, 2H). |
| TDH-503 | ¹H NMR (400 MHz, DMSO) δ 10.64 (s, 1H), 10.34 (s, 1H), 8.69 (s, 1H), 8.29 (s, 1H), 7.92 |
| | (s, 1H), 7.76-7.69 (m, 2H), 7.65-7.57 (m, 2H), 7.54-7.47 (m, 2H), 7.45-7.39 (m, 1H), 7.39- |
| | 7.34 (m, 1H), 7.34-7.25 (m, 3H), 7.21-7.11 (m, 3H), 6.24 (s, 1H), 4.24-4.14 (m, 1H), 4.14- |
| | 4.07 (m, 1H), 3.85 (s, 3H), 3.61-3.57 (m, 2H), 3.20-3.16 (m, 2H), 2.97-2.80 (m, 4H), 2.71- |
| | 2.66 (m, 2H), 2.22-2.15 (m, 2H), 2.14-1.90 (m, 10H), 1.75-1.69 (m, 2H), 1.68-1.51 (m, |
| | 5H), 1.51-1.43 (m, 2H), 1.29-1.19 (m, 2H), 1.19-1.07 (m, 2H). |
| TDH-504 | ¹H NMR (400 MHz, DMSO) δ 10.67 (s, 1H), 10.34 (s, 1H), 8.70 (s, 1H), 8.28 (s, 1H), 7.93 |
| | (s, 1H), 7.76-7.71 (m, 2H), 7.66-7.58 (m, 2H), 7.53-7.46 (m, 2H), 7.46-7.41 (m, 1H), 7.39- |
| | 7.34 (m, 1H), 7.34-7.27 (m, 3H), 7.20-7.13 (m, 3H), 6.25 (s, 1H), 4.23-4.17 (m, 1H), 4.14- |
| | 4.06 (m, 1H), 3.85 (s, 3H), 3.62-3.57 (m, 2H), 3.21-3.15 (m, 2H), 3.01-2.94 (m, 2H), 2.70- |
| | 2.67 (m, 2H), 2.41-2.32 (m, 2H), 2.09-1.90 (m, 12H), 1.77-1.67 (m, 4H), 1.59-1.54 (m, |
| | 2H), 1.52-1.48 (m, 2H), 1.45-1.37 (m, 4H), 1.27-1.22 (m, 3H). |
| TDH-505 | ¹H NMR (400 MHz, DMSO) δ 10.64 (s, 1H), 10.34 (s, 1H), 8.70 (s, 1H), 8.29 (s, 1H), 8.09 |
| | (s, 1H), 7.92 (s, 1H), 7.77-7.71 (m, 2H), 7.65-7.58 (m, 2H), 7.53-7.47 (m, 2H), 7.46-7.40 |
| | (m, 1H), 7.38-7.34 (m, 1H), 7.33-7.29 (m, 2H), 7.20-7.12 (m, 3H), 6.24 (s, 1H), 4.28-4.22 |
| | (m, 1H), 4.21-4.14 (m, 1H), 3.85 (s, 3H), 3.64-3.56 (m, 2H), 3.02-2.94 (m, 2H), 2.89-2.80 |
| | (m, 2H), 2.73-2.64 (m, 2H), 2.43-2.29 (m, 2H), 2.13-1.89 (m, 8H), 1.74-1.60 (m, 4H), 1.44- |
| | 1.36 (m, 3H), 1.32-1.28 (m, 2H), 1.20-1.13 (m, 2H), 1.10-1.01 (m, 2H), 0.93-0.79 (m, 4H). |
| TDH-506 | ¹H NMR (400 MHz, DMSO) δ 10.64 (s, 1H), 10.34 (s, 1H), 8.69 (s, 1H), 8.29 (s, 1H), 8.09 |
| | (s, 1H), 7.92 (s, 1H), 7.75-7.71 (m, 2H), 7.64-7.58 (m, 2H), 7.53-7.46 (m, 2H), 7.46-7.41 |
| | (m, 1H), 7.42-7.36 (m, 1H), 7.32-7.27 (m, 2H), 7.19-7.13 (m, 3H), 6.24 (s, 1H), 4.27-4.23 |
| | (m, 1H), 4.22-4.16 (m, 1H), 3.85 (s, 3H), 3.62-3.57 (m, 4H), 2.93-2.88 (m, 2H), 2.71-2.66 |
| | (m, 2H), 2.21-2.15 (m, 2H), 2.13-1.88 (m, 10H), 1.89-1.81 (m, 2H), 1.77-1.69 (m, 3H), |
| | 1.45-1.41 (m, 2H), 1.33-1.29 (m, 2H), 1.19-1.05 (m, 2H), 0.89-0.81 (m, 4H). |
| TDH-507 | ¹H NMR (400 MHz, DMSO) δ 11.07 (s, 1H), 10.64 (s, 1H), 9.83 (s, 1H), 8.69 (s, 1H), 8.29 |
| | (s, 1H), 7.91 (s, 1H), 7.75-7.68 (m, 2H), 7.66-7.56 (m, 2H), 7.52-7.46 (m, 2H), 7.46-7.39 |
| | (m, 1H), 7.36-7.21 (m, 5H), 7.21-7.13 (m, 2H), 7.03-6.90 (m, 1H), 6.24 (s, 1H), 5.73 (s, |
| | 1H), 5.02-4.92 (m, 1H), 4.41-4.31 (m, 1H), 4.25-4.12 (m, 1H), 3.98-3.87 (m, 1H), 3.74 (s, |
| | 2H), 3.02-2.83 (m, 5H), 2.68-2.53 (m, 5H), 2.21-2.12 (m, 2H), 2.12-1.85 (m, 8H), 1.76- |
| | 1.55 (m, 4H), 1.55-1.43 (m, 2H), 1.41-1.31 (m, 3H), 1.28-1.21 (m, 2H), 1.19-1.05 (m, 2H), |
| | 0.96-0.84 (m, 2H). |
| TDH-508 | ¹H NMR (400 MHz, DMSO) δ 10.71 (s, 1H), 10.34 (s, 1H), 8.69 (s, 1H), 8.29 (s, 1H), 7.92 |
| | (s, 1H), 7.76-7.72 (m, 2H), 7.66-7.60 (m, 2H), 7.52-7.46 (m, 2H), 7.46-7.40 (m, 1H), 7.40- |
| | 7.36 (m, 1H), 7.36-7.31 (m, 1H), 7.31-7.25 (m, 2H), 7.19-7.11 (m, 3H), 6.26 (s, 1H), 4.29- |
| | 4.15 (m, 2H), 3.85 (s, 3H), 3.64-3.57 (m, 2H), 3.05-2.88 (m, 6H), 2.72-2.65 (m, 2H), 2.23- |
| | 2.17 (m, 2H), 2.08-1.91 (m, 11H), 1.81-1.75 (m, 2H), 1.64-1.57 (m, 2H), 1.54-1.37 (m, |
| | 8H), 1.32-1.21 (m, 7H), 0.92-0.84 (m, 2H). |
| TDH-509 | ¹H NMR (400 MHz, DMSO) δ 11.07 (s, 1H), 10.66 (s, 1H), 9.83 (s, 1H), 8.69 (s, 1H), 8.28 |
| | (s, 1H), 7.92 (s, 1H), 7.76-7.70 (m, 2H), 7.66-7.57 (m, 2H), 7.53-7.39 (m, 3H), 7.37-7.23 |
| | (m, 5H), 7.23-7.09 (m, 2H), 7.03-6.94 (m, 1H), 6.24 (s, 1H), 5.73 (s, 1H), 5.02-4.92 (m, |
| | 1H), 4.24-4.18 (m, 1H), 4.14-4.07 (m, 1H), 3.77-3.72 (m, 2H), 3.45-3.41 (m, 2H), 3.02- |
| | 2.95 (m, 2H), 2.91-2.83 (m, 2H), 2.63-2.54 (m, 2H), 2.40-2.32 (m, 2H), 2.07-1.91 (m, 9H), |
| | 1.76-1.64 (m, 4H), 1.48-1.34 (m, 8H), 1.24-1.15 (m, 4H), 0.93-0.81 (m, 2H). |
| TDH-510 | ¹H NMR (400 MHz, DMSO) δ 11.07 (s, 1H), 10.63 (s, 1H), 9.83 (s, 1H), 8.69 (s, 1H), 8.29 |
| | (s, 1H), 7.92 (s, 1H), 7.76-7.70 (m, 2H), 7.67-7.60 (m, 2H), 7.55-7.47 (m, 2H), 7.47-7.39 |
| | (m, 1H), 7.36-7.25 (m, 5H), 7.20-7.11 (m, 2H), 7.02-6.94 (m, 1H), 6.24 (s, 1H), 5.73 (s, |
| | 1H), 5.02-4.93 (m, 1H), 4.42-4.34 (m, 1H), 4.23-4.14 (m, 1H), 4.14-4.04 (m, 1H), 3.99- |
| | 3.90 (m, 1H), 3.73 (s, 2H), 3.00-2.94 (m, 2H), 2.87-2.73 (m, 3H), 2.63-2.55 (m, 2H), 2.39- |
| | 2.31 (m, 2H), 2.09-1.91 (m, 8H), 1.87-1.77 (m, 2H), 1.77-1.68 (m, 2H), 1.68-1.56 (m, 4H), |
| | 1.42-1.34 (m, 2H), 1.27-1.20 (m, 2H), 1.18-1.11 (m, 2H), 0.91-0.83 (m, 2H). |
| TDH-511 | ¹H NMR (400 MHz, DMSO) δ 10.65 (s, 1H), 10.34 (s, 1H), 8.70 (s, 1H), 8.29 (s, 1H), 7.93 |
| | (s, 1H), 7.76-7.69 (m, 2H), 7.65-7.59 (m, 2H), 7.53-7.46 (m, 3H), 7.46-7.41 (m, 1H), 7.41- |
| | 7.37 (m, 1H), 7.37-7.33 (m, 1H), 7.30 (s, 2H), 7.21-7.11 (m, 3H), 6.24 (s, 1H), 4.41-4.33 |
| | (m, 1H), 4.29-4.14 (m, 2H), 4.01-3.93 (m, 1H), 3.85 (s, 3H), 3.63-3.59 (m, 2H), 3.04-2.95 |
| | (m, 4H), 2.70-2.66 (m, 2H), 2.26-2.09 (m, 4H), 2.07-1.88 (m, 10H), 1.75-1.71 (m, 2H), |
| | 1.70-1.62 (m, 4H), 1.44-1.37 (m, 4H), 1.27-1.16 (m, 8H), 0.88-0.83 (m, 2H). |
| TDH-512 | ¹H NMR (400 MHz, DMSO) δ 10.68 (s, 1H), 10.34 (s, 1H), 8.69 (s, 1H), 8.29 (s, 1H), 7.92 |
| | (s, 1H), 7.76-7.72 (m, 2H), 7.65-7.59 (m, 2H), 7.51-7.46 (m, 2H), 7.45-7.42 (m, 1H), 7.40- |
| | 7.35 (m, 1H), 7.35-7.32 (m, 1H), 7.30 (s, 2H), 7.21-7.11 (m, 3H), 6.25 (s, 1H), 4.22-4.17 |
| | (m, 1H), 4.12-4.07 (m, 1H), 3.85 (s, 3H), 3.64-3.58 (m, 4H), 3.13-3.01 (m, 4H), 2.97-2.89 |
| | (m, 4H), 2.70-2.67 (m, 2H), 2.22-2.17 (m, 2H), 2.03-1.91 (m, 10H), 1.83-1.69 (m, 6H), |
| | 1.53-1.41 (m, 6H), 1.24-1.15 (m, 6H), 0.88-0.83 (m, 2H). |
| TDH-513 | ¹H NMR (400 MHz, DMSO) δ 11.11 (s, 1H), 10.63 (s, 1H), 8.69 (s, 1H), 8.30 (s, 1H), 7.92 |
| | (s, 1H), 7.75-7.68 (m, 3H), 7.66-7.57 (m, 2H), 7.53-7.47 (m, 2H), 7.46-7.40 (m, 2H), |
| | 7.29 (s, 2H), 7.21-7.11 (m, 2H), 6.24 (s, 1H), 5.15-5.07 (m, 1H), 4.28-4.23 (m, 1H), |
| | 4.21-4.15 (m, 1H), 3.62-3.57 (m, 2H), 2.99-2.82 (m, 8H), 2.64-2.57 (m, 2H), 2.20-2.13 |
| | (m, 2H), 2.08-1.92 (m, 8H), 1.83-1.76 (m, 2H), 1.73-1.66 (m, 2H), 1.47-1.43 (m, 2H), |
| | 1.36-1.31 (m, 3H), 1.17-1.10 (m, 2H), 0.88-0.83 (m, 2H). |
| TDH-514 | ¹H NMR (400 MHz, DMSO) δ 11.11 (s, 1H), 10.63 (s, 1H), 8.70 (s, 1H), 8.29 (s, 1H), 7.92 |
| | (s, 1H), 7.76-7.71 (m, 3H), 7.66-7.58 (m, 2H), 7.53-7.47 (m, 2H), 7.46-7.40 (m, 2H), |
| | 7.30 (s, 2H), 7.21-7.12 (m, 2H), 6.24 (s, 1H), 5.15-5.06 (m, 1H), 4.27-4.21 (m, 1H), |
| | 4.21-4.16 (m, 1H), 3.64-3.56 (m, 2H), 3.02-2.94 (m, 2H), 2.94-2.78 (m, 6H), 2.68-2.55 |
| | (m, 2H), 2.23-2.14 (m, 2H), 2.06-1.95 (m, 6H), 1.84-1.79 (m, 2H), 1.74-1.63 (m, 3H), |
| | 1.43-1.33 (m, 4H), 1.23-1.15 (m, 4H), 0.90-0.84 (m, 2H). |
| TDH-515 | ¹H NMR (400 MHz, DMSO) δ 10.62 (s, 1H), 10.34 (s, 1H), 8.69 (s, 1H), 8.30 (s, 1H), 7.92 |
| | (s, 1H), 7.77-7.70 (m, 2H), 7.65-7.58 (m, 2H), 7.52-7.47 (m, 2H), 7.46-7.42 (m, 1H), 7.40- |
| | 7.36 (m, 1H), 7.36-7.33 (m, 1H), 7.32-7.25 (m, 2H), 7.19-7.13 (m, 3H), 6.24 (s, 1H), 4.42- |
| | 4.35 (m, 1H), 4.23-4.15 (m, 1H), 4.03-3.95 (m, 1H), 3.85 (s, 3H), 3.64-3.57 (m, 2H), 2.97- |
| | 2.92 (m, 2H), 2.73-2.66 (m, 2H), 2.22-2.14 (m, 2H), 2.10-1.93 (m, 6H), 1.82-1.65 (m, 4H), |
| | 1.61-1.45 (m, 4H), 1.22-1.14 (m, 4H), 0.92-0.78 (m, 4H). |
| TDH-516 | ¹H NMR (400 MHz, DMSO) δ 11.06 (s, 1H), 10.62 (s, 1H), 9.83 (s, 1H), 8.69 (s, 1H), 8.29 |
| | (s, 1H), 7.91 (s, 1H), 7.76-7.68 (m, 2H), 7.65-7.56 (m, 2H), 7.53-7.46 (m, 2H), 7.46-7.38 |
| | (m, 1H), 7.35-7.27 (m, 5H), 7.22-7.10 (m, 2H), 7.04-6.97 (m, 1H), 6.24 (s, 1H), 5.73 (s, |
| | 1H), 5.01-4.93 (m, 1H), 4.44-4.34 (m, 1H), 4.28-4.13 (m, 1H), 4.02-3.93 (m, 1H), 3.75 (s, |
| | 2H), 3.00-2.81 (m, 4H), 2.63-2.58 (m, 2H), 2.18-2.10 (m, 2H), 2.06-1.89 (m, 7H), 1.83- |
| | 1.55 (m, 4H), 0.94-0.86 (m, 2H). |
| TDH-517 | ¹H NMR (400 MHz, DMSO) δ 10.63 (s, 1H), 10.34 (s, 1H), 8.69 (s, 1H), 8.29 (s, 1H), 7.92 |
| | (s, 1H), 7.76-7.69 (m, 2H), 7.65-7.57 (m, 2H), 7.55-7.47 (m, 2H), 7.46-7.41 (m, 1H), 7.41- |
| | 7.36 (m, 1H), 7.36-7.33 (m, 1H), 7.29 (s, 2H), 7.21-7.11 (m, 3H), 6.24 (s, 1H), 4.42-4.33 |
| | (m, 1H), 4.23-4.15 (m, 1H), 4.04-3.94 (m, 1H), 3.85 (s, 3H), 3.63-3.57 (m, 2H), 3.03-2.88 |
| | (m, 5H), 2.72-2.66 (m, 2H), 2.39-2.32 (m, 2H), 2.09-1.90 (m, 7H), 1.78-1.51 (m, 7H), 1.45- |
| | 1.35 (m, 3H), 1.35-1.27 (m, 2H), 0.91-0.81 (m, 2H). |
| TDH-518 | ¹H NMR (400 MHz, DMSO) δ 11.06 (s, 1H), 10.62 (s, 1H), 9.83 (s, 1H), 8.69 (s, 1H), 8.28 |
| | (s, 1H), 7.91 (s, 1H), 7.76-7.69 (m, 2H), 7.69-7.57 (m, 2H), 7.54-7.40 (m, 3H), 7.36-7.24 |
| | (m, 5H), 7.20-7.08 (m, 2H), 7.02-6.94 (m, 1H), 6.23 (s, 1H), 5.73 (s, 1H), 5.01-4.92 (m, |
| | 1H), 4.42-4.34 (m, 1H), 4.22-4.14 (m, 1H), 3.99-3.90 (m, 1H), 3.74 (s, 2H), 3.03-2.82 (m, |
| | 4H), 2.64-2.55 (m, 2H), 2.37-2.27 (m, 2H), 2.09-1.85 (m, 7H), 1.72-1.59 (m, 2H), 1.59- |
| | 1.42 (m, 2H), 1.42-1.31 (m, 2H), 0.98-0.86 (m, 2H). |
| TDH-519 | ¹H NMR (400 MHz, DMSO) δ 11.11 (s, 1H), 10.63 (s, 1H), 8.70 (s, 1H), 8.31 (s, 1H), 7.92 |
| | (s, 1H), 7.76-7.68 (m, 3H), 7.64-7.59 (m, 2H), 7.52-7.42 (m, 4H), 7.29 (s, 2H), 7.20-7.12 |
| | (m, 2H), 6.24 (s, 1H), 5.14-5.07 (m, 1H), 4.26-4.16 (m, 2H), 2.96-2.89 (m, 4H), 2.65- |
| | 2.59 (m, 2H), 2.12-1.95 (m, 10H), 1.87-1.82 (m, 2H), 1.33-1.23 (m, 4H). |
| TDH-520 | ¹H NMR (400 MHz, DMSO) δ 11.11 (s, 1H), 10.63 (s, 1H), 8.70 (s, 1H), 8.30 (s, 1H), 7.92 |
| | (s, 1H), 7.74-7.68 (m, 3H), 7.65-7.58 (m, 2H), 7.52-7.42 (m, 4H), 7.29 (s, 2H), 7.22-7.11 |
| | (m, 2H), 6.24 (s, 1H), 5.16-5.07 (m, 1H), 4.28-4.13 (m, 1H), 3.64-3.58 (m, 2H), 3.04- |
| | 2.95 (m, 2H), 2.93-2.84 (m, 3H), 2.65-2.57 (m, 2H), 2.43-2.34 (m, 2H), 2.11-1.89 (m, |
| | 8H), 1.85-1.79 (m, 2H), 1.49-1.42 (m, 2H), 1.38-1.29 (m, 2H). |
| TDH-521 | ¹H NMR (500 MHz, DMSO) δ 11.02 (s, 1H), 10.66 (s, 1H), 8.73 (s, 1H), 8.27 (s, 1H), 8.02 |
| | (s, 1H), 7.77-7.70 (m, 2H), 7.66-7.58 (m, 2H), 7.54-7.48 (m, 2H), 7.48-7.42 (m, 1H), |
| | 7.34 (s, 2H), 7.20-7.13 (m, 2H), 6.25 (s, 1H), 5.31 (s, 1H), 4.90-4.84 (m, 1H), 4.61-4.49 |
| | (m, 1H), 3.77-3.65 (m, 1H), 3.21-3.04 (m, 6H), 2.88-2.77 (m, 2H), 2.60-2.53 (m, 2H), |
| | 2.46-2.36 (m, 2H), 2.34-2.24 (m, 3H), 2.24-2.15 (m, 1H), 1.95-1.78 (m, 4H), 1.37-1.24 |
| | (m, 2H). |
| TDH-522 | ¹H NMR (300 MHz, DMSO) δ 11.01 (s, 1H), 10.69 (s, 1H), 8.72 (s, 1H), 8.27 (s, 1H), 7.99 |
| | (s, 1H), 7.82-7.68 (m, 2H), 7.68-7.55 (m, 2H), 7.53-7.40 (m, 3H), 7.33 (s, 2H), 7.23-7.08 |
| | (m, 2H), 6.25 (s, 1H), 5.26 (s, 1H), 4.92-4.81 (m, 1H), 4.65-4.45 (m, 1H), 3.75-3.56 (m, |
| | 1H), 3.17-2.99 (m, 6H), 2.94-2.71 (m, 2H), 2.59-2.54 (m, 2H), 2.47-2.37 (m, 2H), 2.37- |
| | 2.13 (m, 4H), 2.01-1.85 (m, 2H), 1.85-1.74 (m, 2H), 1.72-1.63 (m, 2H), 1.34-1.21 (m, 2H). |
| TDH-523 | ¹H NMR (400 MHz, DMSO) δ 10.99 (s, 1H), 10.63 (s, 1H), 8.70 (s, 1H), 8.30 (s, 1H), 7.92 |
| | (s, 1H), 7.76-7.70 (m, 2H), 7.65-7.59 (m, 2H), 7.54-7.46 (m, 2H), 7.46-7.40 (m, 1H), 7.30 |
| | (s, 2H), 7.20-7.12 (m, 2H), 6.24 (s, 1H), 5.25 (s, 1H), 4.89-4.81 (m, 3H), 4.41-4.35 (m, |
| | 1H), 4.24-4.16 (m, 1H), 4.04-3.97 (m, 1H), 3.20-3.13 (m, 2H), 3.07-2.92 (m, 4H), 2.87- |
| | 2.78 (m, 1H), 2.59-2.55 (m, 2H), 2.45-2.37 (m, 1H), 2.24-2.14 (m, 2H), 2.05-1.91 (m, 6H), |
| | 1.74-1.66 (m, 4H), 1.64-1.58 (m, 2H), 1.07-1.01 (m, 2H), 0.93-0.85 (m, 2H). |
| TDH-524 | ¹H NMR (400 MHz, DMSO) δ 10.99 (s, 1H), 10.63 (s, 1H), 8.70 (s, 1H), 8.29 (s, 1H), 7.97- |
| | 7.90 (m, 1H), 7.75-7.67 (m, 2H), 7.64-7.58 (m, 2H), 7.54-7.46 (m, 2H), 7.46-7.38 (m, 1H), |
| | 7.29 (s, 2H), 7.20-7.12 (m, 2H), 6.24 (s, 1H), 5.24 (s, 1H), 4.88-4.80 (m, 1H), 4.41-4.32 |
| | (m, 1H), 4.26-4.20 (m, 1H), 4.02-3.94 (m, 1H), 3.20-3.15 (m, 2H), 3.03-2.98 (m, 2H), 2.86- |
| | 2.78 (m, 1H), 2.59-2.54 (m, 2H), 2.44-2.40 (m, 2H), 2.20-2.11 (m, 2H), 2.08-2.01 (m, 2H), |
| | 1.99-1.88 (m, 3H), 1.76-1.66 (m, 4H), 1.65-1.50 (m, 4H), 1.45-1.39 (m, 2H), 1.37-1.28 |
| | (m, 2H), 0.95-0.77 (m, 4H). |
| TDH-525 | ¹H NMR (300 MHz, DMSO) δ 10.63 (s, 1H), 8.70 (s, 1H), 8.30 (s, 1H), 7.92 (s, 1H), 7.88- |
| | 7.81 (m, 1H), 7.78-7.69 (m, 2H), 7.67-7.55 (m, 2H), 7.54-7.40 (m, 4H), 7.40-7.26 (m, |
| | 3H), 7.21-7.12 (m, 2H), 6.24 (s, 1H), 5.24-5.15 (m, 1H), 5.13 (s, 2H), 4.26-4.15 (m, 1H), |
| | 3.53-3.40 (m, 4H), 3.02 (s, 3H), 2.99-2.90 (m, 2H), 2.82-2.73 (m, 1H), 2.64-2.55 (m, |
| | 1H), 2.49-2.41 (m, 3H), 2.41-2.25 (m, 6H), 2.19-1.90 (m, 7H), 1.71-1.58 (m, 2H). |
| TDH-526 | ¹H NMR (400 MHz, DMSO) δ 10.99 (s, 1H), 10.63 (s, 1H), 8.70 (s, 1H), 8.29 (s, 1H), 7.93 |
| | (s, 1H), 7.76-7.69 (m, 2H), 7.65-7.57 (m, 2H), 7.53-7.46 (m, 2H), 7.46-7.40 (m, 1H), 7.30 |
| | (s, 2H), 7.20-7.13 (m, 2H), 6.24 (s, 1H), 5.25 (s, 1H), 4.90-4.81 (m, 1H), 4.25-4.16 (m, |
| | 1H), 4.13-4.06 (m, 1H), 3.57-3.52 (m, 2H), 3.48-3.44 (m, 2H), 3.03-2.94 (m, 3H), 2.86- |
| | 2.71 (m, 2H), 2.58-2.54 (m, 2H), 2.44-2.28 (m, 9H), 2.19-2.09 (m, 2H), 2.09-2.00 (m, 3H), |
| | 1.99-1.86 (m, 3H), 1.77-1.55 (m, 6H). |
| TDH-527 | ¹H NMR (400 MHz, DMSO) δ 10.99 (s, 1H), 10.63 (s, 1H), 8.69 (s, 1H), 8.29 (s, 1H), 7.92 |
| | (s, 1H), 7.76-7.70 (m, 2H), 7.65-7.58 (m, 2H), 7.54-7.47 (m, 2H), 7.47-7.40 (m, 1H), 7.29 |
| | (s, 2H), 7.21-7.12 (m, 2H), 6.24 (s, 1H), 5.24 (s, 1H), 4.90-4.82 (m, 1H), 4.40-4.33 (m, |
| | 1H), 4.25-4.11 (m, 1H), 4.03-3.94 (m, 1H), 3.19-3.13 (m, 2H), 3.04-2.78 (m, 7H), 2.45- |
| | 2.33 (m, 1H), 2.19-2.16 (m, 2H), 2.12-1.87 (m, 10H), 1.80-1.73 (m, 2H), 1.72-1.66 (m, |
| | 4H), 1.65-1.55 (m, 2H), 1.54-1.43 (m, 2H), 1.37-1.29 (m, 1H), 1.27-1.20 (m, 2H), 1.20- |
| | 1.07 (m, 3H), 1.05-0.97 (m, 1H), 0.95-0.82 (m, 2H). |
| TDH-528 | ¹H NMR (400 MHz, DMSO) δ 10.99 (s, 1H), 10.62 (s, 1H), 8.69 (s, 1H), 8.29 (s, 1H), 7.92 |
| | (s, 1H), 7.77-7.70 (m, 2H), 7.66-7.58 (m, 2H), 7.53-7.46 (m, 2H), 7.46-7.40 (m, 1H), 7.29 |
| | (s, 2H), 7.23-7.12 (m, 2H), 6.24 (s, 1H), 5.24 (s, 1H), 4.91-4.80 (m, 1H), 4.41-4.32 (m, |
| | 1H), 4.24-4.15 (m, 1H), 4.03-3.94 (m, 1H), 3.22-3.10 (m, 3H), 3.05-2.95 (m, 2H), 2.92- |
| | 2.85 (m, 2H), 2.85-2.76 (m, 1H), 2.74-2.67 (m, 1H), 2.59-2.54 (m, 2H), 2.45-2.39 (m, 1H), |
| | 2.38-2.28 (m, 2H), 2.28-2.18 (m, 2H), 2.16-2.08 (m, 2H), 2.08-2.00 (m, 2H), 2.00-1.84 |
| | (m, 8H), 1.82-1.64 (m, 6H), 1.63-1.58 (m, 1H), 1.57-1.53 (m, 2H), 1.50-1.46 (m, 2H), 1.23- |
| | 1.14 (m, 1H), 1.04-0.94 (m, 1H), 0.93-0.81 (m, 2H). |
| TDH-529 | ¹H NMR (400 MHz, DMSO) δ 10.99 (s, 1H), 10.62 (s, 1H), 8.69 (s, 1H), 8.29 (s, 1H), 7.92 |
| | (s, 1H), 7.77-7.67 (m, 2H), 7.65-7.57 (m, 2H), 7.54-7.46 (m, 2H), 7.46-7.39 (m, 1H), 7.29 |
| | (s, 2H), 7.21-7.11 (m, 2H), 6.24 (s, 1H), 5.24 (s, 1H), 4.89-4.81 (m, 1H), 4.23-4.13 (m, |
| | 1H), 3.89-3.81 (m, 1H), 3.80-3.72 (m, 1H), 3.61-3.49 (m, 3H), 3.20-3.14 (m, 2H), 3.16- |
| | 3.04 (m, 2H), 3.04-2.93 (m, 3H), 2.88-2.76 (m, 1H), 2.59-2.53 (m, 3H), 2.47-2.32 (m, 1H), |
| | 2.25-2.14 (m, 2H), 2.07-1.83 (m, 6H), 1.83-1.74 (m, 1H), 1.74-1.66 (m, 2H), 1.65-1.52 |
| | (m, 2H), 1.51-1.38 (m, 2H), 1.38-1.27 (m, 2H). |
| TDH-530 | ¹H NMR (400 MHz, DMSO) δ 11.01 (s, 1H), 10.64 (s, 1H), 8.70 (s, 1H), 8.29 (s, 1H), 7.92 |
| | (s, 1H), 7.82-7.78 (m, 1H), 7.75-7.71 (m, 2H), 7.65-7.58 (m, 3H), 7.54-7.47 (m, 3H), 7.47- |
| | 7.40 (m, 1H), 7.29 (s, 2H), 7.20-7.12 (m, 2H), 6.24 (s, 1H), 5.19-5.09 (m, 1H), 4.55-4.46 |
| | (m, 1H), 4.43-4.32 (m, 1H), 4.25-4.17 (m, 1H), 3.69-3.61 (m, 1H), 3.05-2.95 (m, 2H), 2.95- |
| | 2.84 (m, 1H), 2.70-2.54 (m, 2H), 2.42-2.31 (m, 6H), 2.19-2.08 (m, 2H), 2.08-1.93 (m, 6H), |
| | 1.69-1.61 (m, 2H), 1.39-1.27 (m, 2H), 0.94-0.80 (m, 2H). |
| TDH-531 | ¹H NMR (400 MHz, DMSO) δ 11.08 (s, 1H), 10.62 (s, 1H), 8.69 (s, 1H), 8.09 (s, 1H), 7.92 |
| | (s, 1H), 7.75-7.71 (m, 2H), 7.65-7.58 (m, 2H), 7.52-7.47 (m, 2H), 7.46-7.40 (m, 1H), 7.29 |
| | (s, 2H), 7.19-7.10 (m, 2H), 6.76-6.70 (m, 1H), 6.24 (s, 1H), 5.06-4.97 (m, 1H), 4.39-4.35 |
| | (m, 1H), 4.28-4.17 (m, 3H), 4.00-3.88 (m, 1H), 2.99-2.93 (m, 2H), 2.63-2.57 (m, 2H), 2.39- |
| | 2.33 (m, 2H), 2.12-1.92 (m, 10H), 1.73-1.69 (m, 2H), 1.43-1.327(m, 6H), 0.89-0.69 (m, 2H). |
| TDH-532 | ¹H NMR (400 MHz, DMSO) δ 11.09 (s, 1H), 10.62 (s, 1H), 8.70 (s, 1H), 8.29 (s, 1H), 7.92 |
| | (s, 1H), 7.77-7.70 (m, 2H), 7.65-7.58 (m, 2H), 7.54-7.46 (m, 2H), 7.45-7.40 (m, 1H), 7.29 |
| | (s, 2H), 7.21-7.12 (m, 2H), 6.75-6.69 (m, 1H), 6.24 (s, 1H), 5.05-4.98 (m, 1H), 4.75-4.68 |
| | (m, 1H), 4.41-4.34 (m, 1H), 4.26-4.15 (m, 2H), 4.00-3.92 (m, 1H), 3.02-2.93 (m, 2H), 2.91- |
| | 2.80 (m, 2H), 2.68-2.54 (m, 2H), 2.40-2.31 (m, 6H), 2.06-1.90 (m, 6H), 1.67-1.58 (m, 2H), |
| | 1.48-1.39 (m, 2H), 1.37-1.26 (m, 3H), 0.89-0.81 (m, 2H). |
| TDH-533 | ¹H NMR (400 MHz, DMSO) δ 11.09 (s, 1H), 10.62 (s, 1H), 8.69 (s, 1H), 8.09 (s, 1H), 7.92 |
| | (s, 1H), 7.76-7.70 (m, 2H), 7.66-7.59 (m, 2H), 7.53-7.46 (m, 2H), 7.46-7.40 (m, 1H), 7.29 |
| | (s, 2H), 7.20-7.12 (m, 2H), 6.73 (s, 1H), 6.24 (s, 1H), 5.06-4.96 (m, 1H), 4.40-4.32 (m, |
| | 2H), 4.28-4.13 (m, 3H), 4.00-3.88 (m, 1H), 2.99-2.80 (m, 6H), 2.68-2.55 (m, 3H), 2.21- |
| | 2.16 (m, 2H), 2.03-1.88 (m, 10H), 1.74-1.68 (m, 4H), 1.47-1.40 (m, 2H), 1.36-1.27 (m, |
| | 5H), 0.86-0.83 (m, 2H). |
| TDH-534 | ¹H NMR (400 MHz, DMSO) δ 11.11-10.99 (m, 1H), 10.62 (s, 1H), 8.69 (s, 1H), 8.29 (s, 1H), |
| | 7.90 (s, 1H), 7.77-7.69 (m, 2H), 7.66-7.57 (m, 2H), 7.54-7.45 (m, 2H), 7.45-7.39 (m, 1H), |
| | 7.29 (s, 2H), 7.23-7.11 (m, 2H), 6.72 (s, 1H), 6.24 (s, 1H), 5.36-5.28 (m, 1H), 5.04-4.96 |
| | (m, 1H), 4.85-4.78 (m, 1H), 4.47-4.29 (m, 2H), 4.26-4.14 (m, 2H), 3.99-3.90 (m, 1H), 3.68- |
| | 3.60 (m, 1H), 3.54-3.41 (m, 2H), 3.01-2.78 (m, 4H), 2.78-2.64 (m, 2H), 2.37-2.18 (m, 4H), |
| | 2.10-1.87 (m, 10H), 1.75-1.68 (m, 2H), 1.57-1.52 (m, 2H), 1.48-1.43 (m, 2H), 1.37-1.24 |
| | (m, 4H), 0.90-0.82 (m, 2H). |
| TDH-535 | ¹H NMR (400 MHz, DMSO) δ 10.88 (s, 1H), 10.74 (s, 1H), 8.89 (d, J = 8.3 Hz, 1H), 8.70 (s, |
| | 1H), 8.29 (s, 1H), 7.98-7.89 (m, 3H), 7.77-7.71 (m, 2H), 7.66-7.59 (m, 2H), 7.55-7.47 (m, |
| | 4H), 7.47-7.38 (m, 1H), 7.30 (s, 2H), 7.20-7.12 (m, 2H), 6.27 (s, 1H), 4.86-4.75 (m, 1H), |
| | 4.29-4.17 (m, 1H), 3.70-3.60 (m, 2H), 3.08-2.94 (m, 2H), 2.89-2.70 (m, 2H), 2.62-2.54 |
| | (m, 2H), 2.49-2.41 (m, 3H), 2.41-2.27 (m, 5H), 2.21-2.12 (m, 2H), 2.12-1.87 (m, 6H), 1.75- |
| | 1.60 (m, 2H). |
| TDH-536 | ¹H NMR (400 MHz, DMSO) δ 10.88 (s, 1H), 10.62 (s, 1H), 8.86 (d, J = 8.3 Hz, 1H), 8.70 (s, |
| | 1H), 8.29 (s, 1H), 7.96-7.90 (m, 3H), 7.75-7.69 (m, 2H), 7.65-7.59 (m, 2H), 7.53-7.46 (m, |
| | 4H), 7.46-7.38 (m, 1H), 7.29 (s, 2H), 7.21-7.10 (m, 2H), 6.24 (s, 1H), 4.85-4.77 (m, 1H), |
| | 4.52-4.40 (m, 1H), 4.24-4.14 (m, 1H), 3.01-2.94 (m, 2H), 2.86-2.75 (m, 2H), 2.63-2.53 |
| | (m, 2H), 2.42-2.33 (m, 2H), 2.18-1.93 (m, 8H), 1.85-1.74 (m, 1H), 1.65-1.55 (m, 2H), 1.51- |
| | 1.41 (m, 2H), 1.19-1.07 (m, 2H), 0.94-0.82 (m, 1H). |
| TDH-537 | ¹H NMR (500 MHz, DMSO) δ 10.94 (s, 1H), 10.89 (s, 1H), 8.94 (d, J = 8.3 Hz, 1H), 8.74 (s, |
| | 1H), 8.27 (s, 1H), 7.99-7.93 (m, 3H), 7.81-7.77 (m, 2H), 7.67-7.63 (m, 2H), 7.51-7.47 (m, |
| | 4H), 7.45-7.41 (m, 1H), 7.33 (s, 2H), 7.19-7.13 (m, 2H), 6.32 (s, 1H), 4.85-4.73 (m, 1H), |
| | 4.54-4.39 (m, 1H), 3.54-3.43 (m, 2H), 3.34-3.25 (m, 4H), 3.18-3.02 (m, 2H), 3.02-2.87 |
| | (m, 3H), 2.87-2.77 (m, 3H), 2.60-2.53 (m, 2H), 2.43-2.21 (m, 6H), 2.20-2.10 (m, 3H), 2.09- |
| | 1.89 (m, 6H), 1.87-1.66 (m, 3H), 1.30-1.09 (m, 2H). |
| TDH-538 | ¹H NMR (400 MHz, DMSO) δ 10.88 (s, 1H), 10.62 (s, 1H), 8.86 (d, J = 8.4 Hz, 1H), 8.69 (s, |
| | 1H), 8.29 (s, 1H), 7.96-7.89 (m, 3H), 7.76-7.69 (m, 2H), 7.65-7.58 (m, 2H), 7.54-7.45 (m, |
| | 4H), 7.45-7.40 (m, 1H), 7.29 (s, 2H), 7.21-7.10 (m, 2H), 6.24 (s, 1H), 4.85-4.76 (m, 1H), |
| | 4.22-4.13 (m, 1H), 4.03-3.91 (m, 1H), 3.63-3.54 (m, 3H), 3.47-3.36 (m, 2H), 3.15-3.07 |
| | (m, 1H), 3.04-2.95 (m, 2H), 2.87-2.77 (m, 1H), 2.59-2.54 (m, 2H), 2.23-2.08 (m, 3H), 2.08- |
| | 1.87 (m, 7H), 1.85-1.74 (m, 1H), 1.56-1.39 (m, 2H). |
| TDH-539 | ¹H NMR (400 MHz, DMSO) δ 10.88 (s, 1H), 10.65 (s, 1H), 8.87 (d, J = 8.3 Hz, 1H), 8.69 (s, |
| | 1H), 8.29 (s, 1H), 7.96-7.88 (m, 3H), 7.76-7.70 (m, 2H), 7.66-7.59 (m, 2H), 7.54-7.40 (m, |
| | 5H), 7.29 (s, 2H), 7.21-7.12 (m, 2H), 6.24 (s, 1H), 4.84-4.74 (m, 1H), 4.52-4.43 (m, 1H), |
| | 4.24-4.14 (m, 1H), 3.53-3.45 (m, 2H), 3.10-2.97 (m, 2H), 2.94-2.77 (m, 6H), 2.63-2.54 |
| | (m, 2H), 2.20-2.11 (m, 4H), 2.07-1.90 (m, 9H), 1.85-1.78 (m, 2H), 1.70-1.65 (m, 2H), 1.56- |
| | 1.43 (m, 2H), 1.15-1.08 (m, 2H). |
| TDH-540 | ¹H NMR (400 MHz, DMSO) δ 11.06 (s, 0.3H), 10.75 (s, 0.8H), 8.70 (s, 1H), 8.29 (s, 1H), |
| | 8.09 (s, 1H), 7.93 (s, 1H), 7.78-7.72 (m, 2H), 7.66-7.59 (m, 2H), 7.52-7.45 (m, 2H), 7.45- |
| | 7.37 (m, 2H), 7.34-7.25 (m, 2H), 7.21-7.13 (m, 2H), 6.97-6.91 (m, 0.5H), 6.86-6.78 (m, |
| | 1H), 6.68-6.60 (m, 0.5H), 6.27 (s, 1H), 5.36-5.25 (m, 1H), 4.31-4.15 (m, 3H), 3.67-3.52 |
| | (m, 2H), 2.78-2.63 (m, 2H), 2.39-2.31 (m, 6H), 2.29-2.16 (m, 4H), 2.13-1.92 (m, 8H), 1.74- |
| | 1.63 (m, 5H), 1.45-1.29 (m, 7H), 0.86-0.84 (m, 2H). |
| TDH-541 | ¹H NMR (400 MHz, DMSO) δ 11.13-11.03 (m, 1H), 10.99 (s, 0.5H), 10.62 (s, 1H), 8.69 (s, |
| | 1H), 8.29 (s, 1H), 7.92 (s, 1H), 7.77-7.70 (m, 2H), 7.68-7.57 (m, 2H), 7.52-7.46 (m, 2H), |
| | 7.46-7.39 (m, 1H), 7.29 (s, 2H), 7.20-7.12 (m, 2H), 6.72 (s, 0.5H), 6.24 (s, 1H), 5.06-4.94 |
| | (m, 1H), 4.87-4.76 (m, 1H), 4.42-4.33 (m, 1H), 4.23-4.11 (m, 2H), 3.99-3.89 (m, 1H), 3.88- |
| | 3.77 (m, 1H), 3.62-3.52 (m, 3H), 3.23-3.14 (m, 2H), 3.14-3.04 (m, 2H), 3.03-2.96 (m, 2H), |
| | 2.90-2.75 (m, 2H), 2.44-2.33 (m, 2H), 2.24-2.16 (m, 2H), 2.05-1.97 (m, 4H), 1.85-1.78 |
| | (m, 2H), 1.44-1.37 (m, 2H), 0.90-0.77 (m, 2H). |
| TDH-542 | ¹H NMR (400 MHz, DMSO) δ 10.89 (s, 1H), 10.65 (s, 1H), 8.95-8.87 (m, 1H), 8.70 (s, 1H), |
| | 8.29 (s, 1H), 8.00-7.94 (m, 1H), 7.94-7.88 (m, 2H), 7.77-7.71 (m, 2H), 7.65-7.59 (m, 2H), |
| | 7.59-7.55 (m, 2H), 7.53-7.46 (m, 2H), 7.46-7.39 (m, 1H), 7.30 (s, 2H), 7.21-7.10 (m, 2H), |
| | 6.24 (s, 1H), 4.87-4.78 (m, 1H), 4.27-4.17 (m, 1H), 4.13-4.07 (m, 1H), 3.72-3.59 (m, 2H), |
| | 3.05-2.97 (m, 2H), 2.86-2.75 (m, 1H), 2.62-2.55 (m, 2H), 2.46-2.32 (m, 8H), 2.18-2.10 |
| | (m, 2H), 2.07-1.90 (m, 6H), 1.69-1.59 (m, 2H). |
| TDH-543 | ¹H NMR (400 MHz, DMSO) δ 10.88 (s, 1H), 10.63 (s, 1H), 8.90 (d, J = 8.4 Hz, 1H), 8.70 (s, |
| | 1H), 8.29 (s, 1H), 7.98-7.94 (m, 1H), 7.94-7.90 (m, 1H), 7.90-7.86 (m, 1H), 7.76-7.70 (m, |
| | 2H), 7.65-7.59 (m, 2H), 7.59-7.53 (m, 2H), 7.53-7.46 (m, 2H), 7.46-7.39 (m, 1H), 7.29 (s, |
| | 2H), 7.20-7.12 (m, 2H), 6.24 (s, 1H), 4.85-4.75 (m, 1H), 4.53-4.42 (m, 1H), 4.25-4.16 (m, |
| | 1H), 3.60-3.49 (m, 1H), 3.05-2.96 (m, 2H), 2.88-2.74 (m, 2H), 2.62-2.53 (m, 2H), 2.45- |
| | 2.36 (m, 2H), 2.21-2.07 (m, 3H), 2.07-1.89 (m, 5H), 1.87-1.73 (m, 1H), 1.72-1.54 (m, 2H), |
| | 1.51-1.40 (m, 2H), 1.23-1.05 (m, 3H). |
| TDH-544 | ¹H NMR (400 MHz, DMSO) δ 10.88 (s, 1H), 10.66 (s, 1H), 8.90 (d, J = 8.4 Hz, 1H), 8.69 (s, |
| | 1H), 8.29 (s, 1H), 7.98-7.93 (m, 1H), 7.91 (s, 1H), 7.90-7.84 (m, 1H), 7.76-7.70 (m, 2H), |
| | 7.66-7.60 (m, 2H), 7.59-7.53 (m, 2H), 7.52-7.47 (m, 2H), 7.45-7.40 (m, 1H), 7.29 (s, 2H), |
| | 7.20-7.12 (m, 2H), 6.25 (s, 1H), 4.85-4.78 (m, 1H), 4.22-4.14 (m, 1H), 3.59-3.46 (m, 2H), |
| | 2.91-2.87 (m, 2H), 2.83-2.75 (m, 2H), 2.74-2.66 (m, 2H), 2.60-2.56 (m, 2H), 2.35-2.27 |
| | (m, 2H), 2.25-2.18 (m, 2H), 2.15-2.11 (m, 2H), 2.05-2.01 (m, 2H), 1.99-1.96 (m, 2H), 1.96- |
| | 1.93 (m, 2H), 1.93-1.87 (m, 4H), 1.83-1.76 (m, 2H), 1.56-1.51 (m, 4H), 1.48-1.44 (m, 2H), |
| | 1.09-0.99 (m, 2H). |
| TDH-545 | ¹H NMR (400 MHz, DMSO) δ 10.88 (s, 1H), 10.62 (s, 1H), 8.89 (d, J = 8.4 Hz, 1H), 8.69 (s, |
| | 1H), 8.29 (s, 1H), 7.99-7.93 (m, 1H), 7.92 (s, 1H), 7.91-7.88 (m, 1H), 7.76-7.69 (m, 2H), |
| | 7.63-7.55 (m, 4H), 7.51-7.46 (m, 2H), 7.46-7.41 (m, 1H), 7.29 (s, 2H), 7.20-7.12 (m, 2H), |
| | 6.24 (s, 1H), 4.86-4.77 (m, 1H), 4.22-4.14 (m, 1H), 4.04-3.92 (m, 1H), 3.61-3.56 (m, 3H), |
| | 3.50-3.40 (m, 2H), 3.03-2.95 (m, 2H), 2.86-2.76 (m, 1H), 2.59-2.55 (m, 3H), 2.24-2.07 |
| | (m, 4H), 2.03-1.90 (m, 6H), 1.85-1.76 (m, 1H), 1.55-1.41 (m, 2H). |
| TDH-546 | ¹H NMR (400 MHz, DMSO) δ 10.88 (s, 1H), 10.72 (s, 1H), 8.91 (d, J = 8.3 Hz, 1H), 8.69 (s, |
| | 1H), 8.29 (s, 1H), 7.97-7.93 (m, 1H), 7.92 (s, 1H), 7.89-7.86 (m, 1H), 7.78-7.72 (m, 2H), |
| | 7.66-7.61 (m, 2H), 7.59-7.53 (m, 2H), 7.52-7.46 (m, 2H), 7.46-7.39 (m, 1H), 7.29 (s, 2H), |
| | 7.20-7.12 (m, 2H), 6.26 (s, 1H), 4.85-4.77 (m, 1H), 4.54-4.43 (m, 1H), 4.25-4.16 (m, 1H), |
| | 3.59-3.47 (m, 2H), 3.15-2.75 (m, 7H), 2.67-2.55 (m, 3H), 2.25-2.14 (m, 4H), 2.06-1.91 |
| | (m, 9H), 1.86-1.75 (m, 2H), 1.72-1.67 (m, 2H), 1.59-1.41 (m, 2H). |
| TDH-547 | ¹H NMR (400 MHz, DMSO) δ 11.01 (s, 1H), 10.63 (s, 1H), 8.69 (s, 1H), 8.29 (s, 1H), 7.92 |
| | (s, 1H), 7.82-7.76 (m, 1H), 7.76-7.70 (m, 2H), 7.66-7.58 (m, 3H), 7.54-7.45 (m, 3H), 7.47- |
| | 7.39 (m, 1H), 7.29 (s, 2H), 7.20-7.11 (m, 2H), 6.24 (s, 1H), 5.19-5.10 (m, 1H), 4.56-4.45 |
| | (m, 2H), 4.38 (d, J = 17.5 Hz, 1H), 4.25-4.15 (m, 1H), 3.58-3.46 (m, 1H), 3.02-2.95 (m, |
| | 2H), 2.96-2.86 (m, 1H), 2.84-2.70 (m, 2H), 2.66-2.59 (m, 2H), 2.46-2.35 (m, 2H), 2.12- |
| | 1.89 (m, 8H), 1.67-1.58 (m, 2H), 1.48-1.40 (m, 2H), 1.17-1.09 (m, 2H). |
| TDH-548 | ¹H NMR (400 MHz, DMSO) δ 11.01 (s, 1H), 10.66 (s, 1H), 8.69 (s, 1H), 8.29 (s, 1H), 7.92 |
| | (s, 1H), 7.81-7.76 (m, 1H), 7.75-7.71 (m, 2H), 7.66-7.58 (m, 3H), 7.53-7.46 (m, 3H), 7.46- |
| | 7.40 (m, 1H), 7.29 (s, 2H), 7.20-7.12 (m, 2H), 6.25 (s, 1H), 5.18-5.09 (m, 1H), 4.54-4.45 |
| | (m, 2H), 4.43-4.32 (m, 1H), 4.23-4.15 (m, 1H), 3.55-3.48 (m, 1H), 3.06-2.95 (m, 1H), 2.95- |
| | 2.85 (m, 3H), 2.83-2.70 (m, 2H), 2.70-2.56 (m, 3H), 2.45-2.37 (m, 1H), 2.37-2.18 (m, 3H), |
| | 2.18-2.08 (m, 2H), 2.08-2.00 (m, 3H), 1.96-1.87 (m, 5H), 1.83-1.72 (m, 2H), 1.67-1.58 |
| | (m, 1H), 1.58-1.49 (m, 4H), 1.49-1.41 (m, 2H), 1.14-0.99 (m, 2H). |
| TDH-549 | ¹H NMR (400 MHz, DMSO) δ 11.01 (s, 1H), 10.63 (s, 1H), 8.69 (s, 1H), 8.29 (s, 1H), 7.92 |
| | (s, 1H), 7.81-7.77 (m, 1H), 7.75-7.69 (m, 2H), 7.66-7.59 (m, 3H), 7.54-7.47 (m, 3H), 7.46- |
| | 7.40 (m, 1H), 7.29 (s, 2H), 7.20-7.12 (m, 2H), 6.24 (s, 1H), 5.18-5.09 (m, 1H), 4.55-4.46 |
| | (m, 1H), 4.42-4.33 (m, 1H), 4.22-4.13 (m, 1H), 4.04-3.93 (m, 1H), 3.62-3.55 (m, 3H), 3.03- |
| | 2.96 (m, 2H), 2.96-2.87 (m, 1H), 2.69-2.52 (m, 5H), 2.45-2.33 (m, 1H), 2.24-2.12 (m, 2H), |
| | 2.06-1.87 (m, 7H), 1.83-1.67 (m, 1H), 1.54-1.39 (m, 2H). |
| TDH-550 | ¹H NMR (400 MHz, DMSO) δ 11.01 (s, 1H), 10.69 (s, 1H), 8.69 (s, 1H), 8.30 (s, 1H), 7.91 |
| | (s, 1H), 7.81-7.77 (m, 1H), 7.75-7.72 (m, 2H), 7.66-7.60 (m, 3H), 7.52-7.46 (m, 3H), 7.46- |
| | 7.40 (m, 1H), 7.29 (s, 2H), 7.20-7.11 (m, 2H), 6.26 (s, 1H), 5.19-5.11 (m, 1H), 4.55-4.44 |
| | (m, 2H), 4.44-4.34 (m, 1H), 4.24-4.14 (m, 1H), 3.55-3.49 (m, 1H), 3.08-2.97 (m, 1H), 2.97- |
| | 2.80 (m, 6H), 2.69-2.57 (m, 2H), 2.44-2.32 (m, 1H), 2.20-2.13 (m, 3H), 2.10-1.89 (m, 10H), |
| | 1.85-1.76 (m, 2H), 1.71-1.65 (m, 2H), 1.54-1.43 (m, 1H), 1.16-1.05 (m, 4H). |
| TDH-551 | ¹H NMR (400 MHz, DMSO) δ 10.64 (s, 1H), 10.58 (s, 1H), 8.69 (s, 1H), 8.29 (s, 1H), 8.24 |
| | (s, 1H), 7.92 (s, 1H), 7.76-7.70 (m, 2H), 7.70-7.64 (m, 2H), 7.64-7.58 (m, 2H), 7.52-7.46 |
| | (m, 2H), 7.46-7.40 (m, 1H), 7.33-7.27 (m, 3H), 7.20-7.13 (m, 2H), 6.24 (s, 1H), 4.23-4.14 |
| | (m, 1H), 3.90-3.83 (m, 2H), 3.70-3.54 (m, 2H), 3.02-2.91 (m, 2H), 2.84-2.76 (m, 2H), 2.45- |
| | 2.30 (m, 9H), 2.12-1.89 (m, 7H), 1.68-1.58 (m, 2H). |
| TDH-552 | ¹H NMR (400 MHz, DMSO) δ 10.64 (s, 1H), 10.58 (s, 1H), 8.69 (s, 1H), 8.29 (s, 1H), 8.23 |
| | (s, 1H), 7.92 (s, 1H), 7.76-7.70 (m, 2H), 7.68-7.59 (m, 4H), 7.53-7.46 (m, 2H), 7.46-7.40 |
| | (m, 1H), 7.33-7.24 (m, 3H), 7.19-7.11 (m, 2H), 6.24 (s, 1H), 4.24-4.14 (m, 1H), 3.90-3.82 |
| | (m, 2H), 3.00-2.95 (m, 2H), 2.82-2.76 (m, 2H), 2.42-2.34 (m, 2H), 2.14-1.91 (m, 8H), 1.83- |
| | 1.69 (m, 2H), 1.66-1.54 (m, 2H), 1.46-1.39 (m, 2H), 1.22-1.09 (m, 3H). |
| TDH-553 | ¹H NMR (400 MHz, DMSO) δ 10.73 (s, 1H), 10.58 (s, 1H), 8.69 (s, 1H), 8.29 (s, 1H), 8.23 |
| | (s, 1H), 7.92 (s, 1H), 7.79-7.73 (m, 2H), 7.68-7.61 (m, 4H), 7.51-7.46 (m, 2H), 7.46-7.40 |
| | (m, 1H), 7.34-7.25 (m, 3H), 7.20-7.12 (m, 2H), 6.27 (s, 1H), 4.26-4.16 (m, 1H), 3.90-3.84 |
| | (m, 2H), 2.95-2.88 (m, 2H), 2.81-2.77 (m, 2H), 2.35-2.12 (m, 5H), 2.12-1.86 (m, 11H), |
| | 1.86-1.68 (m, 4H), 1.66-1.53 (m, 5H), 1.53-1.42 (m, 3H), 1.13-1.05 (m, 2H). |
| TDH-554 | ¹H NMR (400 MHz, DMSO) δ 10.63 (s, 1H), 10.57 (s, 1H), 8.69 (s, 1H), 8.29 (s, 1H), 8.23 |
| | (s, 1H), 7.92 (s, 1H), 7.76-7.70 (m, 2H), 7.70-7.64 (m, 2H), 7.64-7.58 (m, 2H), 7.53-7.46 |
| | (m, 2H), 7.46-7.40 (m, 1H), 7.35-7.26 (m, 3H), 7.21-7.11 (m, 2H), 6.24 (s, 1H), 4.21-4.13 |
| | (m, 1H), 3.90-3.82 (m, 2H), 3.62-3.54 (m, 3H), 3.03-2.95 (m, 2H), 2.83-2.77 (m, 2H), 2.58- |
| | 2.52 (m, 4H), 2.24-2.14 (m, 2H), 2.07-1.64 (m, 8H), 1.58-1.40 (m, 2H). |
| TDH-555 | ¹H NMR (400 MHz, DMSO) δ 10.66 (s, 1H), 10.58 (s, 1H), 8.69 (s, 1H), 8.29 (s, 1H), 8.24 |
| | (s, 1H), 7.92 (s, 1H), 7.76-7.71 (m, 2H), 7.68-7.60 (m, 4H), 7.53-7.46 (m, 2H), 7.46-7.40 |
| | (m, 1H), 7.33-7.26 (m, 3H), 7.20-7.11 (m, 2H), 6.25 (s, 1H), 4.23-4.15 (m, 1H), 3.89-3.84 |
| | (m, 2H), 3.03-2.84 (m, 6H), 2.82-2.78 (m, 2H), 2.21-2.16 (m, 2H), 2.14-1.90 (m, 10H), |
| | 1.87-1.78 (m, 2H), 1.77-1.61 (m, 4H), 1.60-1.40 (m, 2H), 1.16-1.03 (m, 4H). |

### Evaluation of the Compounds of the Present Invention

Measurement of Binding Affinity with MLKL Receptor (Competitive Binding Assay)

The binding affinity was evaluated by measuring the Kd value in the following method.

To prepare kinase-tagged T7 phage strains in an E. coli host derived from the BL21 strain, E. coli were grown to log-phase and infected with T7 phage and incubated with shaking at 32° C. until lysis. The lysates were centrifuged and filtered to remove cell debris. For kinase production, it was expressed in human embryonic kidney cell line (HEK 293 cell line), and subsequently tagged with DNA for qPCR detection.

Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Binding affinities (Kds) were determined using an 11-point 3-fold compound dilution series with three DMSO control points.

All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

### Degradation Assay

MLKL degradation ability was evaluated in the following method.

1 mL of Human liver cancer cell line (HepG2 cell line) or human skin cell line (HaCaT cell line) was equally distributed into each well at 2.0×10⁵ cells/mL in a 12-well cell culture plate. On the next day, the medium was replaced with a medium made so that the final concentration of test compound was 1 or 10 μM (1 mL/well), and incubated in 5% CO₂, at 37° C. for 20-24 hours (Thermo scientific, Forma steri-cycle).

For protein preparation after incubation, cells were washed with 1×TBST, and 60 μL of RIPA lysis buffer containing Halt™ Protease and Phosphatase Inhibitor Cocktail was dispensed into each well. After scraping the cells with a scraper, the cells were recovered, and the pallet was homogenized and reacted on ice for 30 minutes. After the cells were centrifuged at 13,000 rpm, 10 minutes, 4° C., the supernatant was transferred to a new E-tube. For protein quantification, after dispensing sample and BCA (0-4 mg/ml) in a 96 well plate, 100 μL of BCA was dispensed and reacted in a 37° C. incubator for 30 minutes. After measuring the absorbance (SpectraMax, M5e, 562 nm), the amount of the sample was adjusted with the same concentration of protein and 4× Laemmli sample buffer, DW, and reacted at 100° C. for 5 minutes.

After assembling the MP TGX Precast Gel in the Mini-PROTEAN Tetra Cell, the same protein (10-15 μg) was added in an 8-10 μL volume per well, 1×Tris-Glycine Buffer was added, and loading was performed. After loading was completed, transfer was performed using Pierce G2 Fast Blotter and Pierce 1-Step Transfer Buffer. After completion of the transfer, 10 ml of SuperBlock™ Blocking Buffer was dispensed and reacted at room temperature for 10 minutes. 5% BSA containing the primary antibody was dispensed and reacted over night at 4° C. The next day, washing was performed three times with 1×TBST at room temperature for 10 minutes. 5% BSA containing the secondary antibody was dispensed and reacted at room temperature for 1 hour. Washing was performed three times with 1×TBST at room temperature for 10 minutes. The amount of protein expression was measured using chemiluminescence (LuminoGraph, ATTO).

### Necroptosis Inhibition Assay

Necroptosis inhibition was evaluated by the following method.

80 μL of FADD gene-defective human T lymphocyte cells (FADD−/− Jurkat cells) were equally distributed into each well at 5.0×10⁵ cells/mL in a white 96-well plate with a transparent bottom. 20 μL of the medium containing test compound was treated in each well so that the final concentration of the compound was 0.1, 1, and 10 μM, and reacted at room temperature for 30 minutes. After the 30-minute reaction was over, TNF (tumor necrosis factor)-α was treated in the corresponding well so that the final concentration of TNF-α was 15 ng/mL in order to induce necroptosis. It was cultured in a 5% CO₂, 37° C. incubator for 20 to 24 hours.

After incubation, the 96-well plate was left at room temperature for 30 minutes. After 30 minutes, Promega's CellTiter-Glo 2.0 reagent was diluted 1:3 with PBS and 100 μL of the diluted solution was added to each well to confirm cell viability by measuring the amount of ATP. After the treatment, the plate was shielded from light and incubated at room temperature for 2 minutes with orbital shaking, and then left at room temperature for 10 minutes without shaking. After measuring the luminescence at 0.5 seconds per well using a plate scanner, the cytotoxicity and necroptosis inhibition rate for the compound were calculated using the measured values.

The results of MLKL binding affinity (kd) evaluation, degradation evaluation, and necroptosis inhibition evaluation performed with the examples or comparative examples prepared according to the present invention are summarized in Table 4 below.

TABLE 4

| | | | | Necroptosis | |
|---|---|---|---|---|---|
| | Competitive | Degradation assay | | inhibition assay | |
| | Binding assay | (Degradation %) | | (Rescue %) | |
| Compound | (nM) | 1 μM | 10 μM | 1 μM | 10 μM |
| Ceritinib (C2) | >10000 | | | | |
| Crizotinib (C1) | 67 | | | | |
| TDH-001 | 94 | ND | ND | ND | ND |
| TDH-002 | >10000 | ND | ND | 6.35 | ND |
| TDH-027 | 710 | ND | ND | 9.68 | ND |
| TDH-034 | 6300 | ND | ND | ND | ND |
| TDH-053 | 780 | ND | ND | 20.47 | ND |
| TDH-058 | 190 | ND | ND | ND | ND |
| TDH-066 | 650 | | | | |
| TDH-067 | >10000 | | | | |
| TDH-068 | 380 | | | | |
| TDH-069 | 0.22 | | | | |
| TDH-070 | 110 | | | | |
| TDH-071 | 8300 | | | | |
| TDH-072 | 30 | ND | ND | 12.25 | 1.53 |
| TDH-073 | 210 | ND | ND | ND | 3.26 |
| TDH-074 | 74 | ND | ND | 14.54 | 3.61 |
| TDH-075 | 140 | ND | ND | 10.02 | 9.05 |
| TDH-076 | 2200 | ND | ND | ND | 2.53 |
| TDH-078 | 90 | ND | ND | ND | ND |
| TDH-079 | 45 | ND | ND | ND | 0.94 |
| TDH-080 | 120 | ND | ND | ND | ND |
| TDH-081 | 50 | ND | ND | 6.8 | ND |
| TDH-082 | 70 | ND | ND | ND | ND |
| TDH-083 | 84 | ND | ND | ND | ND |
| TDH-084 | 19 | ND | ND | 14.91 | 3.95 |
| TDH-085 | 12 | ND | ND | 5.02 | ND |
| TDH-086 | 41 | ND | ND | 2.91 | ND |
| TDH-087 | 38 | ND | ND | ND | ND |
| TDH-088 | 33 | ND | ND | 4.62 | ND |
| TDH-089 | 56 | ND | ND | ND | ND |
| TDH-092 | 120 | ND | ND | 2.1 | ND |
| TDH-095 | 26 | ND | ND | ND | ND |
| TDH-096 | 4 | 60.02 | 68.41 | 32.98 | 30.53 |
| TDH-097 | 0.73 | | | ND | ND |
| TDH-098 | 200 | | | 3.54 | ND |
| TDH-099 | 11 | | | 18.18 | ND |
| TDH-100 | 4.4 | | | 18.57 | 12.01 |
| TDH-101 | 1.9 | 35.85 | 29.42 | 50.77 | 18.45 |
| TDH-102 | 2.1 | 12.94 | 42.92 | 39.17 | 42.99 |
| TDH-103 | 1.4 | 5.5 | 52.04 | 34.93 | 59.65 |
| TDH-104 | 48 | 14.79 | 62.43 | 25.6 | 22.88 |
| TDH-105 | 6.1 | 43.02 | 40.8 | 27.07 | 15.46 |
| TDH-106 | 6500 | | | 13.76 | 34.33 |
| TDH-107 | 560 | ND | ND | 24.34 | 50.16 |
| TDH-108 | 4.4 | 48.06 | 43.66 | 75.49 | 43.94 |
| TDH-109 | 2.8 | 48.55 | 56.91 | 71.74 | 48.69 |
| TDH-110 | 5.2 | 9.84 | 46.89 | 55.93 | 72.8 |
| TDH-111 | 5.3 | 4.54 | 28.85 | 45.17 | 78.08 |
| TDH-112 | 2 | | | 20.24 | ND |
| TDH-113 | 2.3 | 24.54 | 19.24 | 24.96 | 5.16 |
| TDH-114 | 0.83 | 8.68 | 62.79 | 13.68 | 38.3 |
| TDH-115 | 6.3 | 10.31 | 24.21 | 12.42 | 3.59 |
| TDH-116 | 2 | ND | ND | 16.8 | 42.8 |
| TDH-119 | 7.9 | ND | ND | ND | ND |
| TDH-120 | 1.7 | ND | ND | 10.65 | ND |
| TDH-121 | 4.6 | ND | ND | 2.96 | ND |
| TDH-122 | 1.8 | ND | ND | 20.17 | ND |
| TDH-123 | 0.89 | ND | ND | 8.69 | 8.92 |
| TDH-124 | 12 | ND | 8.02 | 21.88 | 27.22 |
| TDH-125 | 3.2 | 30.1 | 69.02 | 21.27 | 54.55 |
| TDH-126 | 9.2 | 31.29 | 81.59 | 19.35 | 58.35 |
| TDH-127 | 5.6 | 4.37 | 43.64 | 34.64 | 49.65 |
| TDH-128 | 14 | 11 | 68.52 | 26.22 | 56.56 |
| TDH-129 | 7.1 | 32.68 | 57.68 | 49.22 | 67.79 |
| TDH-130 | 11 | 58.3 | 24.87 | 40.74 | 27.69 |
| TDH-131 | 3.9 | 51.13 | 47.86 | 24.56 | 0.96 |
| TDH-132 | 1.2 | 40.32 | 50.03 | 75.06 | 54.76 |
| TDH-133 | 0.78 | 53.05 | 40.54 | 91.87 | 68.81 |
| TDH-134 | 3 | 14.36 | 54.11 | 50.04 | 86.09 |
| TDH-135 | 0.91 | 23.45 | 57.93 | 46.82 | 33.77 |
| TDH-136 | 4.6 | 50.37 | 48.83 | 48.59 | 40.66 |
| TDH-137 | 2.8 | 54.49 | 54.94 | 76.05 | 63.68 |
| TDH-138 | 4.2 | 40.46 | 25.69 | 70.53 | 60.57 |
| TDH-139 | 4.9 | ND | 78.56 | 18.11 | 61.89 |
| TDH-140 | 9.2 | 41.81 | 83.53 | 21.32 | 81.41 |
| TDH-141 | 8.1 | 17.47 | 58.93 | 2.72 | 10.22 |
| TDH-142 | 4.4 | 19.42 | 53.09 | 12.69 | 50.51 |
| TDH-143 | 4.5 | 64 | 85.29 | 20.58 | 64.35 |
| TDH-144 | 2.5 | 58.79 | 86.28 | 15.23 | 68.71 |
| TDH-145 | 2.5 | 36.97 | 55.66 | 51.47 | 40.41 |
| TDH-146 | 5.4 | 65.04 | 60.65 | 42.2 | 9.61 |
| TDH-147 | 24 | 38.91 | 60.57 | 84.3 | 45.37 |
| TDH-148 | 1.4 | 46.29 | 48.24 | 54.25 | 15.48 |
| TDH-149 | 40 | 48.51 | 77.36 | 82.1 | 55.19 |
| TDH-150 | 43 | 66.97 | 53.7 | 53.91 | 39.57 |
| TDH-151 | 12 | 84.58 | 77.74 | 85.85 | 60.12 |
| TDH-152 | 2.7 | 51.96 | 35.05 | 63.2 | 41.21 |
| TDH-153 | 9.2 | 37.66 | 24.63 | 37.21 | 16.52 |
| TDH-154 | 3.8 | 25.56 | 28.19 | 37.01 | 23.23 |
| TDH-155 | 9.5 | 31.35 | 63.85 | 31.1 | 64.6 |
| TDH-156 | 53 | 31.35 | 18.75 | 19.9 | 21.77 |
| TDH-157 | 47 | 62.45 | 64.73 | 37.7 | 45.37 |
| TDH-158 | 35 | 49.22 | 39.81 | ND | ND |
| TDH-159 | 18 | ND | 29.21 | 33.57 | 34.1 |
| TDH-160 | 9.4 | ND | ND | 3.59 | ND |
| TDH-161 | 29 | ND | ND | 25.81 | 35.81 |
| TDH-162 | 11 | ND | ND | 36.55 | 26.25 |
| TDH-163 | 45 | ND | ND | 30.03 | 28.56 |
| TDH-164 | 20 | 55.2 | 46.68 | 27.84 | 10.5 |
| TDH-165 | 0.9 | 62.49 | 61.44 | 52.37 | 4.66 |
| TDH-166 | 3.4 | 18.17 | 60.05 | 27.44 | 65.57 |
| TDH-167 | 1.7 | 36.7 | 60.91 | 45.5 | 38.58 |
| TDH-168 | 7.1 | ND | 24.52 | 31.24 | 40.55 |
| TDH-169 | 2 | 71.77 | 58.09 | 68.09 | 50.74 |
| TDH-170 | 4.6 | 60.57 | 58.27 | 44.08 | 23.26 |
| TDH-171 | 2.3 | 64.64 | 64.5 | 93.81 | 65.91 |
| TDH-172 | 26 | 59.24 | 57.1 | 85.53 | 74.34 |
| TDH-173 | 17 | 61.08 | 75.11 | 47.27 | 35.55 |
| TDH-174 | 2.2 | 49.38 | 52.62 | 16.47 | 42.59 |
| TDH-175 | 7.3 | 48.93 | 43.56 | 18.82 | 35.89 |
| TDH-176 | 22 | 34.07 | 23.76 | 44.28 | 35.42 |
| TDH-177 | 84 | 36.03 | 75.8 | 41.3 | 48.7 |
| TDH-178 | 73 | 72.12 | 84.5 | 24.45 | 28.25 |
| TDH-179 | 31 | 78.61 | 61.56 | 23.48 | 14.8 |
| TDH-180 | 8.6 | | | | |
| TDH-181 | 5200 | | | | |
| TDH-182 | 14 | | | | |
| TDH-183 | 2 | | | | |
| TDH-184 | 16 | | | | |
| TDH-185 | 14 | | | | |
| TDH-186 | 75 | 14.8 | 21.54 | 35.73 | 35.74 |
| TDH-187 | 150 | 27.67 | ND | 28.68 | 29.31 |
| TDH-188 | 3.5 | 7.04 | 5.3 | 45.68 | 42.08 |
| TDH-189 | 2.3 | 29.01 | 12.04 | 69.2 | 32.25 |
| TDH-190 | 13 | ND | 4.57 | 39.55 | 43.59 |
| TDH-191 | 4.9 | 44.99 | 5.25 | 77.85 | 49.93 |
| TDH-192 | 6.1 | ND | ND | 50.37 | 45.32 |
| TDH-193 | 6.1 | ND | 11.22 | 43.97 | 40.55 |
| TDH-194 | 6.7 | 33.12 | 18.24 | 67.94 | 58.3 |
| TDH-195 | 6.9 | 63.85 | 29.78 | 80.66 | 39.24 |
| TDH-196 | 0.97 | 62.01 | 51.12 | 60.75 | 34 |
| TDH-197 | 0.74 | 58.62 | 23.6 | 56.07 | 32.02 |
| TDH-198 | 1.3 | 69.49 | 41.71 | 79.13 | 31.65 |
| TDH-199 | 1.7 | 61.77 | 38.35 | 36.93 | 18 |
| TDH-200 | 1 | 74.03 | 60.19 | 76.41 | 44.64 |
| TDH-201 | 1.3 | 85.96 | 82.98 | 91.53 | 62.79 |
| TDH-202 | 2.1 | 65.39 | 53.78 | 60.16 | 29.13 |
| TDH-203 | 3.2 | 61.41 | 34.74 | 64.32 | 40.46 |
| TDH-204 | 4.2 | 70 | 53.96 | 61.24 | 38.71 |
| TDH-205 | 6.7 | 57.56 | 36.05 | 59.88 | 41.34 |
| TDH-206 | 240 | 21.78 | 14.94 | ND | ND |
| TDH-207 | 210 | 17.98 | 24.18 | 28.69 | 32.45 |
| TDH-208 | 350 | ND | ND | ND | 15.28 |
| TDH-209 | 140 | 7.36 | ND | 7.73 | 18.12 |
| TDH-210 | 300 | ND | ND | 7.5 | 10.08 |
| TDH-211 | 6.3 | 9.7 | 29.62 | 29.87 | 41.58 |
| TDH-212 | 11 | ND | ND | 24.34 | 49.14 |
| TDH-213 | 330 | 4.45 | 8.28 | 18.34 | 44.98 |
| TDH-214 | 180 | ND | ND | 26.74 | 48.41 |
| TDH-215 | 91 | ND | ND | 15.33 | 21.83 |
| TDH-216 | 43 | ND | ND | 21.7 | 38.63 |
| TDH-217 | 66 | 11.62 | 31.11 | 24.76 | 48.87 |
| TDH-218 | 46 | ND | ND | 27.92 | 40.68 |
| TDH-221 | 27 | ND | ND | 53.65 | 54.23 |
| TDH-222 | 16 | 37.4 | 30.04 | 45.31 | 34.2 |
| TDH-223 | 23 | 29.38 | 12.51 | 34.97 | 23.64 |
| TDH-224 | 19 | 49.26 | 56.93 | 63.63 | 67.66 |
| TDH-225 | 8 | 36.29 | 25.24 | 55.04 | 49.01 |
| TDH-226 | 16 | ND | 36.06 | 36.17 | 17.82 |
| TDH-227 | 12 | | | | |
| TDH-228 | 1.2 | | | | |
| TDH-229 | 1.1 | | | | |
| TDH-230 | 140 | ND | ND | 7.4 | 10.64 |
| TDH-231 | 140 | ND | ND | 15.47 | 11.4 |
| TDH-232 | 260 | ND | ND | 3.46 | ND |
| TDH-233 | 160 | 15.59 | 16.2 | 23.37 | 4.71 |
| TDH-234 | 1.6 | 64.08 | 54.89 | 35.55 | 26.56 |
| TDH-235 | 8.5 | 45.01 | 49.89 | 7.64 | 19.27 |
| TDH-236 | 4.9 | 32.16 | 27.8 | 42.35 | 46.48 |
| TDH-237 | 1.8 | 56.98 | 65.18 | 67.77 | 34.51 |
| TDH-238 | 1.7 | 30.48 | 18.31 | 43.63 | 31.99 |
| TDH-239 | 3.8 | 53.9 | 65.78 | 40.3 | 23.29 |
| TDH-240 | 4.2 | 88.47 | — | 43.23 | 14.03 |
| TDH-241 | 2.9 | 64.55 | 64.03 | 50.09 | 28.85 |
| TDH-242 | 2.5 | 75.75 | 66.84 | 71.4 | 44.8 |
| TDH-243 | 0.94 | 57.14 | 54.82 | 61.25 | 53.31 |
| TDH-244 | 1.3 | 75.95 | 66.16 | 92.11 | 65.67 |
| TDH-245 | 3.6 | 67.67 | 62.75 | 65.75 | 14.51 |
| TDH-246 | 3.3 | 66.35 | 46.78 | 60.97 | 26.97 |
| TDH-247 | 4.6 | 64.32 | 45.04 | 40.99 | 33.69 |
| TDH-248 | 8.5 | 47.44 | 14.59 | 72.38 | 51.46 |
| TDH-249 | 5.4 | 35.81 | 28.42 | 45.5 | 44.81 |
| TDH-250 | 4.9 | 78.33 | 71.54 | 69.46 | 46.6 |
| TDH-251 | 1.8 | 72.87 | 63.25 | 84.44 | 74.61 |
| TDH-252 | 3.9 | 76.56 | 84.2 | 124.11 | 73.38 |
| TDH-253 | 10 | 80.74 | 59.15 | 65.01 | 36.1 |
| TDH-254 | 8 | 19.16 | 25.97 | 24.05 | 15.37 |
| TDH-255 | 41 | 35.59 | 34.35 | 46.68 | 32.39 |
| TDH-256 | 78 | 36.84 | 24.99 | 45.87 | 44.55 |
| TDH-257 | 96 | 7.56 | 26.63 | 24.61 | 21.71 |
| TDH-258 | 78 | 13.9 | 10.67 | 33.73 | 24.69 |
| TDH-259 | 50 | 18.61 | 30.36 | ND | ND |
| TDH-260 | 18 | 38.65 | 53.35 | 14.46 | 13.52 |
| TDH-261 | 43 | 34.54 | 42.25 | 15.5 | 14.28 |
| TDH-262 | 65 | 58.43 | 75.8 | 22.59 | 4.02 |
| TDH-263 | 7.4 | 76.27 | 71.82 | 16.13 | 57.97 |
| TDH-264 | 3.4 | 65.05 | 53.98 | 23.38 | 40.72 |
| TDH-265 | 4.4 | 11.99 | 49.83 | 19.18 | 41.68 |
| TDH-266 | 6 | 18.14 | 40.08 | 35.24 | 68.2 |
| TDH-267 | 5.7 | ND | 37.75 | 21.58 | 60.07 |
| TDH-268 | 6.9 | 7.05 | 35.64 | 21.45 | 51.22 |
| TDH-269 | 5.4 | ND | 12.14 | 35.71 | 81.5 |
| TDH-270 | 6.1 | 19.32 | 39.14 | 38.56 | 99.37 |
| TDH-271 | 85 | 1.47 | 4.22 | 45.32 | 76.94 |
| TDH-272 | 13 | 23.54 | 44.2 | 60.73 | 72.92 |
| TDH-273 | 3.5 | 77.5 | 61.88 | 54.02 | 34.08 |
| TDH-274 | 5.2 | 56.81 | 38.77 | 77.01 | 57.62 |
| TDH-275 | 4.9 | 57.84 | 63.11 | 69.59 | 69.98 |
| TDH-276 | 3.6 | 78.62 | 75.61 | 75.36 | 70.49 |
| TDH-277 | 1.5 | 40.52 | 53.39 | 43.59 | 13.88 |
| TDH-278 | 1.4 | 64.63 | 75.28 | 63.64 | 39.23 |
| TDH-279 | 5 | 38.42 | 15.86 | 24.78 | 44.14 |
| TDH-280 | 9.3 | 53.99 | 39.22 | 33.16 | 19.54 |
| TDH-281 | 5.7 | 45.01 | 10.1 | 29.65 | 26.95 |
| TDH-282 | 5.6 | 60.51 | 58.02 | 77.92 | 67.84 |
| TDH-283 | 2.1 | 4.29 | 31.1 | 44.29 | 54.92 |
| TDH-284 | 2.6 | 42.05 | 58.09 | 75.95 | 52.77 |
| TDH-285 | 13 | 56.55 | 50.66 | 36.72 | 40.43 |
| TDH-286 | 12 | 71.83 | 71.94 | 88.45 | 79.75 |
| TDH-287 | 3.7 | 75.04 | 65.94 | 71.05 | 72.7 |
| TDH-288 | 5.2 | 75.31 | 89.16 | 70.93 | 66.54 |
| TDH-289 | 2.2 | 66.33 | 43.03 | 67.46 | 61.24 |
| TDH-290 | 5.3 | 56.44 | 44.97 | 90.57 | 84.93 |
| TDH-291 | 6.7 | 70.94 | 48.11 | 87.96 | 64.92 |
| TDH-292 | 2.3 | 79.96 | 81.68 | 78.15 | 73.02 |
| TDH-293 | 2.3 | 76.09 | 71.04 | 108.66 | 87.1 |
| TDH-294 | 4.7 | 25.53 | 23.63 | 67.12 | 33.24 |
| TDH-295 | 8.1 | 70.94 | 57.99 | 63.46 | 37.73 |
| TDH-296 | 22 | 75.78 | 62.01 | 25.58 | 26.91 |
| TDH-297 | 13 | 76.86 | 70.16 | 74.28 | 63.92 |
| TDH-298 | 9.8 | 45.8 | 78.83 | 41.64 | 66.6 |
| TDH-299 | 7.2 | 79.84 | 89.54 | 75.61 | 62.7 |
| TDH-300 | 9 | 54.31 | 69.42 | 55.98 | 38.09 |
| TDH-301 | 2.9 | 76.3 | 89.97 | 61.06 | 26.7 |
| TDH-302 | 8.6 | 96.64 | 85.06 | 54.15 | 51.56 |
| TDH-303 | 13 | 82.82 | 82.05 | 10.34 | 17.69 |
| TDH-304 | 2.9 | 85.02 | 84.91 | 38.26 | 35.22 |
| TDH-305 | 2.8 | 72.47 | 50.39 | 66.84 | 52.42 |
| TDH-306 | 5.4 | 62.87 | 45.31 | 34.35 | 43.09 |
| TDH-307 | 1.6 | 57.38 | 24.79 | 83.43 | 73.58 |
| TDH-308 | 1.7 | 55.12 | 69.64 | 76.28 | 56.73 |
| TDH-309 | 2.2 | 76.12 | 77.55 | 75.1 | 78.37 |
| TDH-310 | 2.6 | 35.97 | 27.19 | 40.08 | 41.99 |
| TDH-311 | 5.3 | 69.38 | 52.38 | 57.09 | 39.01 |
| TDH-312 | 6.9 | 59.87 | 40.64 | 75.61 | ND |
| TDH-313 | 140 | | | | |
| TDH-314 | 0.62 | | | | |
| TDH-315 | 0.94 | | | | |
| TDH-316 | 0.36 | | | | |
| TDH-317 | 0.62 | | | | |
| TDH-318 | 0.37 | | | | |
| TDH-319 | 0.82 | | | | |
| TDH-320 | 1.7 | | | | |
| TDH-321 | 2 | | | | |
| TDH-322 | 1.4 | | | | |
| TDH-323 | 1.2 | | | | |
| TDH-324 | 1.8 | | | | |
| TDH-325 | 1.1 | | | | |
| TDH-326 | 10 | | | | |
| TDH-327 | 6800 | | | | |
| TDH-328 | 1.8 | 69.07 | 58.46 | 52.22 | 31.65 |
| TDH-329 | 1.7 | | | | |
| TDH-330 | 0.65 | 59.64 | 49.16 | 54.05 (1.1□M) | 51.95 |
| TDH-331 | 2.6 | 54.86 | 60.37 | 49.38 (1.1□M) | 47.13 |
| TDH-332 | 2.6 | 61.62 | 63.17 | 68.62 (1.1□M) | 37.17 |
| TDH-333 | 4.1 | 57.66 | 71.32 | 58.92 (1.1□M) | 36.65 |
| TDH-334 | 0.82 | 75.98 | 83.61 | 86.58 (1.1□M) | 50.45 |
| TDH-335 | 68 | | | | |
| TDH-336 | 1.4 | 73.04 | 66.71 | 39.89 | 19.67 |
| TDH-337 | 2.5 | 46.82 | 65.07 | 32.31 | 45.5 |
| TDH-338 | 5.5 | 59.83 | 79.19 | 50.8 | 50.29 |
| TDH-339 | 13 | 71.36 | 51.92 | 12.23 | 12.31 |
| TDH-340 | 7.5 | 73.39 | 73.33 | 42.94 | 43.81 |
| TDH-341 | 3.5 | 70.78 | 51.56 | 38.23 | 24.8 |
| TDH-342 | 5.8 | 70.74 | 54.03 | 28.05 | 17.55 |
| TDH-343 | 11 | 31.43 | 43.78 | 12.75 | 14.42 |
| TDH-344 | 13 | 64.15 | 47.75 | 14.48 | 17.63 |
| TDH-345 | 1.5 | 87.91 | 80.66 | 29.41 | 19.45 |
| TDH-346 | 1.2 | 85.62 | 81.91 | 57.41 | 24.57 |
| TDH-347 | 1.9 | 77.96 | 85.06 | 23.88 | 12.87 |
| TDH-348 | 1.6 | 48.66 | 81.15 | 61.43 | 29.04 |
| TDH-349 | 3.7 | 93.71 | 92.68 | 45.54 | 42.11 |
| TDH-350 | 3.7 | 86.13 | 88.73 | 39.64 | 34.84 |
| TDH-351 | 6.1 | 62.4 | 24.61 | 0.74 | 3.39 |
| TDH-352 | 5.4 | 50.93 | 42.16 | 11.36 | 5.3 |
| TDH-353 | 26 | 75.35 | 86.12 | 55.34 (1.1□M) | 49.23 |
| TDH-354 | 1.4 | 68.16 | 66.72 | 31.96 | 35.2 |
| TDH-355 | 1.9 | 59.51 | 61.42 | 22.40 | 26.54 |
| TDH-356 | 4.6 | 22.83 | ND | 5.27 | 0.50 |
| TDH-357 | 0.55 | 84.79 | 76.36 | 82.13 (1.1□M) | ND |
| TDH-358 | 0.5 | 91.6 | 86.73 | 100.10 (1.1□M) | 39.65 |
| TDH-359 | 1.7 | 92.98 | 85.17 | 67.68 (1.1□M) | 40.02 |
| TDH-360 | 3.3 | 87.25 | 73.06 | 55.49 (1.1□M) | 38.05 |
| TDH-361 | 1.7 | 51.1 | 48.59 | 19.10 | 10.01 |
| TDH-362 | 5.5 | 69.87 | 69.78 | 17.89 | 16.12 |
| TDH-363 | 2.9 | 76.83 | 69.84 | 32.65 | 27.11 |
| TDH-364 | 5.3 | 72.07 | 65.89 | 22.41 | 17.66 |
| TDH-365 | 3.9 | 72.59 | 82.21 | 19.16 | 16.51 |
| TDH-366 | 1.2 | 87.27 | 80.23 | 45.56 | 22.39 |
| TDH-367 | 0.82 | 84.28 | 80.64 | 36.86 | 21.34 |
| TDH-368 | 1 | 86.09 | 88.6 | 40.51 | 5.97 |
| TDH-369 | 1.1 | 73.33 | 94 | 48.04 | 22.49 |
| TDH-370 | 0.28 | 91.76 | 85.83 | 52.00 | 24.38 |
| TDH-371 | 3.7 | 82.38 | 83.26 | 35.76 | 25.74 |
| TDH-372 | 12 | 78.14 | 81.79 | 26.36 | 28.54 |
| TDH-373 | 1.4 | 87.78 | 86.6 | 62.68 | 40.09 |
| TDH-374 | 2.7 | 93.73 | 91.38 | 39.14 | 32.53 |
| TDH-375 | 2.3 | 93.86 | 86.43 | 30.67 | 20.44 |
| TDH-376 | 0.54 | 93.13 | 86.31 | 76.20 | 30.33 |
| TDH-377 | 0.61 | 89.95 | 82.82 | 62.50 | 27.37 |
| TDH-378 | 2 | 88.62 | 80.01 | 49.61 | 28.89 |
| TDH-379 | 2.9 | 69.78 | 88.75 | 36.70 | 28.98 |
| TDH-380 | 1.5 | 86.88 | 95.41 | 38.37 | 35.96 |
| TDH-381 | 1.5 | 78.38 | 77.07 | 32.48 | 28.99 |
| TDH-382 | 0.42 | 81.82 | 81.68 | 59.24 | 22.12 |
| TDH-383 | 0.42 | 79.04 | 81.95 | 46.33 | 25.19 |
| TDH-384 | 1.9 | 81.59 | 58.63 | 39.08 | 9.67 |
| TDH-385 | 0.76 | 94.85 | 85.11 | 53.62 | 14.09 |
| TDH-386 | 0.93 | 79.68 | 71.14 | 34.89 | 20.76 |
| TDH-387 | 1.6 | 85.77 | 66.9 | 37.82 | 25.00 |
| TDH-388 | 2.3 | 68.53 | 80.04 | 22.66 | 22.49 |
| TDH-390 | 2.6 | 80.07 | 93.00 | 46.50 | 36.13 |
| TDH-393 | 0.99 | 73.39 | 82.10 | 27.21 | 38.01 |
| TDH-394 | 2.8 | 60.83 | 94.76 | 47.22 | 53.54 |
| TDH-395 | 6.1 | 88.76 | 89.67 | 34.73 | 37.15 |
| TDH-396 | 4.2 | 3.42 | 88.49 | 53.20 | 46.28 |
| TDH-397 | 2.7 | 87.39 | 79.77 | 53.83 | 37.24 |
| TDH-398 | 8.9 | 88.25 | 83.83 | 36.81 | 30.39 |
| TDH-399 | 7.6 | 74.40 | 71.63 | 34.21 | 27.35 |
| TDH-400 | 4.4 | 82.22 | 74.77 | 27.80 | 28.44 |
| TDH-401 | 6.8 | 74.27 | 72.99 | 33.10 | 28.25 |
| TDH-402 | 7.7 | 65.36 | 48.34 | 23.40 | 19.16 |
| TDH-403 | 2.8 | 79.40 | 83.30 | 43.50 | 29.42 |
| TDH-404 | 1.1 | 45.30 | 79.08 | 26.83 | 49.11 |
| TDH-405 | 3.2 | 70.59 | 64.30 | 36.97 | 37.28 |
| TDH-406 | 6.5 | 77.69 | 60.15 | 44.99 | 39.97 |
| TDH-407 | 7.5 | 83.70 | 79.77 | 52.02 | 42.95 |
| TDH-408 | 0.57 | 77.02 | 44.73 | 26.68 | 19.89 |
| TDH-409 | 1.6 | 81.20 | 71.90 | 39.62 | 24.45 |
| TDH-410 | 1.1 | 85.29 | 65.91 | 48.51 | 27.36 |
| TDH-411 | 5.7 | 53.86 | 44.78 | 9.91 | 10.81 |
| TDH-412 | 2.5 | 72.66 | 72.89 | 24.36 | 29.48 |
| TDH-413 | 0.92 | 71.41 | 57.83 | 24.79 | 18.59 |
| TDH-414 | 0.79 | 75.28 | 74.89 | 17.52 | 13.19 |
| TDH-415 | 1.7 | 89.91 | 90.14 | 41.74 | 40.81 |
| TDH-416 | 2.4 | 93.24 | 93.04 | 52.88 | 43.06 |
| TDH-417 | 3.5 | 87.84 | 63.85 | 24.41 | 14.37 |
| TDH-418 | 4.0 | 79.05 | 58.33 | 28.11 | 17.31 |
| TDH-419 | 5.9 | 82.06 | 64.50 | 26.56 | 18.52 |
| TDH-420 | 10 | 74.57 | 51.38 | 8.39 | 2.00 |
| TDH-421 | 5.3 | 86.78 | 86.81 | 37.14 | 35.74 |
| TDH-422 | 4.1 | 91.95 | 89.12 | 50.21 | 40.34 |
| TDH-423 | 9.8 | 61.54 | 39.01 | ND | ND |
| TDH-424 | 5.1 | | | | |
| TDH-425 | 0.34 | 50.12 | 88.68 | 67.90 | ND |
| TDH-426 | 1.2 | 18.62 | 57.41 | 55.42 | ND |
| TDH-427 | 2.4 | 75.94 | 59.59 | 5.57 | 9.73 |
| TDH-428 | 4.1 | 58.66 | 85.95 | 27.27 | 9.58 |
| TDH-429 | 2.3 | 77.13 | 63.72 | 18.34 | 11.03 |
| TDH-430 | 2.2 | 65.96 | 36.66 | 15.88 | 14.13 |
| TDH-431 | 1 | 86.88 | 76.78 | 37.24 | 17.64 |
| TDH-432 | 0.9 | 89.17 | 73.05 | 12.47 | 0.07 |
| TDH-433 | 2.1 | 74.16 | 46.20 | 16.00 | 11.01 |
| TDH-434 | 1.4 | 85.08 | 74.66 | 28.72 | 20.87 |
| TDH-435 | 1.8 | 79.73 | 64.92 | 18.26 | 11.49 |
| TDH-436 | 3.1 | 85.98 | 71.25 | 41.81 | 21.38 |
| TDH-437 | 1.3 | 87.34 | 79.50 | 42.82 | 23.20 |
| TDH-438 | 4.5 | 86.72 | 78.91 | 26.37 | 13.48 |
| TDH-439 | 0.91 | 86.02 | 91.86 | 41.96 | 43.64 |
| TDH-440 | 0.51 | 81.51 | 84.75 | 65.67 | 24.42 |
| TDH-441 | 0.69 | 87.59 | 87.7 | 71.58 | −15.73 |
| TDH-442 | 0.53 | 80.37 | 87.27 | 58.16 | 21.7 |
| TDH-443 | 0.52 | 84.23 | 86.93 | 59.05 | −65.46 |
| TDH-444 | 300 | | | | |
| TDH-445 | 0.6 | 20.29 | 27.38 | 12.07 | 19.83 |
| TDH-446 | 0.22 | 57.4 | 17.94 | 34.93 | 8.47 |
| TDH-447 | 0.17 | 60.39 | 14.41 | 33.69 | 14.38 |
| TDH-448 | 0.38 | 48.26 | 7.49 | 27.91 | 20.07 |
| TDH-449 | Pomalidomide | | | | |
| TDH-450 | Negative PROTAC | | | | |
| TDH-451 | 0.54 | 64.37 | 50.39 | 19.55 | 17.67 |
| TDH-452 | 1.8 | 61.94 | 52.12 | 38.27 | 26.02 |
| TDH-453 | 0.64 | 74.72 | 92.96 | 51.32 | 49.41 |
| TDH-454 | 2 | 87.16 | 90.47 | 65.44 | 39.99 |
| TDH-455 | 1200 | | | | |
| TDH-456 | 0.61 | | | | |
| TDH-457 | 3.7 | | | | |
| TDH-458 | 0.28 | | | | |
| TDH-459 | 6.5 | | | | |
| TDH-460 | 2.8 | | | | |
| TDH-461 | 20 | | | | |
| TDH-462 | 4 | | | | |
| TDH-463 | 0.35 | | | | |
| TDH-464 | 0.2 | | | | |
| TDH-465 | 0.25 | | | | |
| TDH-466 | 0.41 | 60.76 | 86.28 | 54.2 | 26.69 |
| TDH-467 | 0.5 | 93.95 | 86.45 | 54.19 | −9.09 |
| TDH-468 | 1.7 | | | | |
| TDH-469 | 0.57 | | | | |
| TDH-470 | 3.8 | | | | |
| TDH-471 | 27 | | | | |
| TDH-472 | 1.6 | 50.63 | 69.94 | 27.45 | 1.07 |
| TDH-473 | 1.7 | 63.51 | 73.71 | 28.37 | −13.59 |
| TDH-474 | 0.95 | 54.33 | 68.31 | 25.61 | 5.98 |
| TDH-475 | 0.34 | 81.77 | 60.49 | 31.75 | 7.21 |
| TDH-476 | 0.32 | 87.94 | 73.34 | 47.52 | 25.23 |
| TDH-477 | 0.26 | 92.48 | 90.67 | 62.38 | 21.83 |
| TDH-478 | 0.26 | 89.29 | 86.88 | 92.11 | 43.19 |
| TDH-479 | 5.6 | | | | |
| TDH-480 | 0.48 | 46.42 | 73 | 35.76 | 39.78 |
| TDH-481 | 8.8 | 74.5 | 63.36 | 17.64 | 9.86 |
| TDH-482 | 1.2 | 91.19 | 88.98 | 78.94 | 42.16 |
| TDH-483 | 2.5 | 86.34 | 84.54 | 62.95 | 37.9 |
| TDH-484 | 0.92 | 89.17 | 91.3 | 66.68 | 37.9 |
| TDH-485 | 10 | 87.46 | 93.6 | 61.51 | 50.03 |
| TDH-486 | 2.5 | 85.37 | 88.15 | 77.72 | 34.5 |
| TDH-487 | 4.3 | 77.25 | 89.93 | 59.49 | 58.14 |
| TDH-488 | 1.9 | | | | |
| TDH-489 | 1.2 | | | | |
| TDH-490 | 400 | | | | |
| TDH-491 | 78 | 19.04 | 14.39 | 0.07 | −24.78 |
| TDH-492 | 39 | 13.74 | 14.89 | 10.33 | 4.12 |
| TDH-493 | 91 | ND | ND | 1.49 | −3.98 |
| TDH-494 | 0.98 | 80.66 | 88.82 | 43.07 | −7.06 |
| TDH-495 | 1.3 | 81.34 | 85.73 | 42.19 | 17.62 |
| TDH-496 | 2.2 | 87.42 | 81.07 | 27.23 | 7.85 |
| TDH-497 | 0.61 | 73.89 | 57.10 | 13.48 | −1.14 |
| TDH-498 | 3.7 | 86.68 | 89.27 | 23.9 | 16.6 |
| TDH-499 | 0.41 | 90.60 | 91.41 | 45.83 | 11.8 |
| TDH-500 | 0.64 | 80.41 | 83.21 | 25.16 | 0.16 |
| TDH-501 | 0.95 | 93.47 | 96.40 | 92.23 | 71.17 |
| TDH-502 | 0.42 | 85.39 | 91.54 | 54.75 | 66.18 |
| TDH-503 | 0.44 | 93.09 | — | 94.80 | 14.61 |
| TDH-504 | 0.23 | 93.87 | 94.41 | 94.61 | 33.71 |
| TDH-505 | 0.36 | 90.52 | 94.63 | 102.00 | 76.87 |
| TDH-506 | 0.6 | 88.53 | 88.65 | 75.45 | 48.09 |
| TDH-507 | 1.1 | 82.75 | 86.37 | 26.03 | 14.19 |
| TDH-508 | 0.4 | 87.16 | 90.95 | 48.15 | 39.79 |
| TDH-509 | 0.78 | 76.18 | 73.70 | 19.06 | 5.55 |
| TDH-510 | 0.47 | 79.77 | 84.86 | 28.63 | −91.81 |
| TDH-511 | 0.24 | 81.08 | 91.73 | 32.32 | 46.16 |
| TDH-512 | 0.74 | 51.41 | 80.48 | 24.43 | 52.33 |
| TDH-513 | 3.9 | 46.49 | 75.71 | 14.47 | −61.68 |
| TDH-514 | 0.9 | 55.53 | 84.44 | 24.14 | −67.06 |
| TDH-515 | 1.4 | 95.21 | 89.06 | 68.04 | 15.68 |
| TDH-516 | 9.7 | 81.44 | 77.67 | 17.46 | 5.65 |
| TDH-517 | 0.62 | 93.97 | 86.64 | 58.11 | 10.78 |
| TDH-518 | 4.3 | 89.77 | 88.59 | 12.44 | −8.57 |
| TDH-519 | 7.1 | 89.40 | 86.43 | 4.80 | 17.07 |
| TDH-520 | 19 | 92.44 | 94.32 | 63.56 | 36.11 |
| TDH-521 | 1.5 | 91.54 | 78.62 | 0.16 | 1.33 |
| TDH-522 | 1.3 | 84.27 | 51.16 | 21.52 | −21.15 |
| TDH-523 | 1 | 89.24 | 77.14 | 35.34 | 16.27 |
| TDH-524 | 0.99 | 89.35 | 83.06 | 48.61 | 15.69 |
| TDH-525 | Negative PROTAC | | | | |
| TDH-526 | 1.2 | 67.25 | 81.42 | 57.37 | 22.77 |
| TDH-527 | 1.2 | 71.10 | 42.46 | 42.79 | −12.98 |
| TDH-528 | 0.18 | 63.93 | 56.11 | 40.69 | 0.38 |
| TDH-529 | 0.25 | 24.89 | 66.48 | 36.72 | 15.86 |
| TDH-530 | 0.21 | 54.30 | 61.10 | 29.18 | 30.78 |
| TDH-531 | 0.28 | 67.64 | 53.49 | 16.12 | 15.34 |
| TDH-532 | 1.4 | 73.88 | 38.71 | 21.37 | 24.82 |
| TDH-533 | 0.47 | 33.35 | 29.05 | 11.86 | 22.99 |
| TDH-534 | 0.24 | 49.96 | 24.88 | 21.33 | 22.25 |
| TDH-535 | 0.96 | 57.69 | 44.41 | 20.08 | 23.78 |
| TDH-536 | 0.22 | 46.11 | 56.40 | 31.84 | 21.93 |
| TDH-537 | 0.61 | 64.82 | 86.14 | 32.07 | 30.76 |
| TDH-538 | 0.31 | 66.34 | 54.70 | 24.02 | 21.52 |
| TDH-539 | 0.68 | 70.67 | 89.04 | 46.99 | 30.33 |
| TDH-540 | 0.37 | 81.44 | 56.99 | 20.79 | 17.37 |
| TDH-541 | 0.34 | 78.24 | 69.17 | 24.95 | 24.58 |
| TDH-542 | 0.53 | 84.12 | 72.45 | 24.80 | 29.40 |
| TDH-543 | 0.23 | 76.24 | 80.46 | 37.86 | 26.19 |
| TDH-544 | 0.5 | 69.45 | 82.68 | 35.33 | 31.42 |
| TDH-545 | 0.28 | 65.64 | 74.02 | 32.16 | 41.07 |
| TDH-546 | 0.4 | 70.89 | 81.41 | 31.08 | 28.77 |
| TDH-547 | 0.43 | 93.36 | 68.21 | 35.18 | 19.80 |
| TDH-548 | 0.19 | 89.05 | 85.68 | 64.86 | 31.41 |
| TDH-549 | 0.64 | 84.54 | 34.92 | 35.84 | 25.21 |
| TDH-550 | 0.53 | 88.92 | 86.09 | 47.98 | 20.62 |
| TDH-551 | 0.3 | 88.03 | 83.80 | 40.61 | 10.85 |
| TDH-552 | 0.41 | 92.21 | 75.10 | 31.97 | 6.25 |
| TDH-553 | 0.31 | 84.21 | 91.57 | 58.63 | −20.56 |
| TDH-554 | 0.43 | 83.95 | 70.50 | 39.53 | 14.16 |
| TDH-555 | 0.22 | 78.56 | ND | 47.10 | −51.39 |

* ND: Not detected.

As described above, the pharmacological activity on related diseases mediated by MLKL Signaling can be judged through the results of measuring the ability to bind to the MLKL receptor, the ability to degrade MLKL protein, and/or the ability to inhibit necroptosis.

Looking at Table 4, it can be seen that most of the compounds of the present invention showed excellent binding ability to the MLKL receptor and showed significant activity against MLKL degradation and necroptosis inhibition. In detail, it was confirmed that some compounds showed a high necroptosis inhibition rate of 50% or more.

Therefore, through these results, it can be seen that a pharmaceutical composition for MLKL-mediated diseases comprising the compound(s) of Examples according to the present invention as an active ingredient can be provided.

All documents mentioned herein are incorporated herein by reference as if their contents were set forth herein. When introducing elements of the present invention or preferred embodiment(s) thereof, the articles “a”, “an”, “the” and “said” refer to one or more elements is intended to mean that there is. The terms “comprising”, “including” and “having” are intended to be inclusive and mean that there may be additional elements other than those listed. Although the invention has been described in terms of a particular embodiment or embodiments, it should not be construed as limiting the details of these embodiments.

1. A compound of Chemical Formula 1: 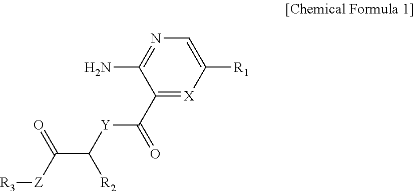
or a pharmaceutically acceptable salt thereof,
in the Chemical Formula 1
X is N or CH,
Y is S, O, NH or —N(C₁₋₆alkyl)-,
Z is a direct bond, —NH—, —N—(C₁₋₄alkyl)-, or 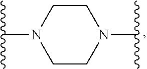 [SMILES: *N1CCN(*)CC1]
R₁ is C₁₋₁₀alkyl, aryl, heteroaryl, cycloalkyl, heterocycloalkyl, or -A-(B)-R₄, wherein R₁ is unsubstituted or substituted with at least one selected from C₁₋₆alkyl, halo-C₁₋₆alkyl, halogen, amino, alkoxy, nitro, cyano, —NRₐC(═O)R_{b}, —NRₐC(═O)NRₐR_{b}, —NRₐC(═O)OR_{b}, or —NRₐSO₂R_{b}, wherein Rₐ and R_{b} are the same or different and each independently hydrogen, C₁₋₆alkyl, aryl, heteroaryl, cycloalkyl, or heterocycloalkyl,
A is a 4-6-membered heteroaryl or heterocycloalkyl containing one or more N,
B is a direct bond (i.e. does not exist), or a 4-7-membered heterocycloalkyl containing one or more N,
R₄ is hydrogen, (lower)alkyl, halo-C₁₋₆alkyl, —C(═O)Rₐ, —C(═O)ORₐ, —PO₃Rₐ, —PO(ORₐ)(OR_{b}), —C(═O)NRₐR_{b}, —NRₐSO₂R_{b}, —SO₂Rₐ, —S(O)Rₐ, or —SO(N)Rₐ, wherein Rₐ and R_{b} are the same or different and each independently C₁₋₆alkyl, aryl, heteroaryl, cycloalkyl, or heterocycloalkyl,
R₂ is hydrogen, C₁₋₆alkyl, C₁₋₆alkoxy, halo-C₁₋₆alkyl, aryl, heteroaryl, or cycloalkyl, wherein the alkyl, alkoxy, aryl, heteroaryl or cycloalkyl is unsubstituted or substituted with at least one selected from C₁₋₆alkyl, halo-C₁₋₆alkyl, halogen, amino, alkoxy, nitro, cyano, —NRₐC(═O)R_{b}, —NRₐC(═O)NRₐR_{b}, —NRₐC(═O)OR_{b}, or —NRₐSO₂R_{b}, wherein Rₐ and R_{b} are the same or different and each independently hydrogen, halogen, amino, C₁₋₆alkyl, halo-C₁₋₆alkyl, aryl, heteroaryl, cycloalkyl or heterocycloalkyl, and
R₃ is C₁₋₁₀alkyl, aryl, heteroaryl, cycloalkyl, or heterocycloalkyl, wherein the aryl, heteroaryl, cycloalkyl, or heterocycloalkyl is unsubstituted or substituted with at least one selected from C₁₋₆alkyl, halogen, halo-C₁₋₆alkyl, C₁₋₆alkoxy, CN, —C(═O)Rₐ, —C(═O)ORₐ, —C(O)NRₐR_{b}, —SO₂Rₐ, —S(O)Rₐ, —SO(N)Rₐ, NO₂, —N(Rₐ)(R_{b}), aryl, aryl substituted with methyl, heterocycloalkyl, or heterocycloalkyl substituted with methyl, wherein Rₐ and R_{b} are the same or different and each independently hydrogen, halogen, amino, C₁₋₆alkyl, halo-C₁₋₆alkyl, aryl, heteroaryl, cycloalkyl or heterocycloalkyl.

2. The compound of claim 1 or a pharmaceutically acceptable salt thereof, wherein
X is N or CH,
Y is S, O, NH or —N(C₁₋₆alkyl)-,
Z is a direct bond, —NH—, —N—(C₁₋₄alkyl)-, or 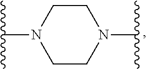 [SMILES: *N1CCN(*)CC1]
R₁ is aryl, heteroaryl, or -A-(B)-R₄, wherein A is a 4-6-membered heteroaryl or heterocycloalkyl containing one or more N; B is a direct bond, or a 4-7-membered heterocycloalkyl containing one or more N; R₄ is hydrogen, (lower)alkyl, halo-C₁₋₆alkyl, —C(═O)Rₐ, —C(═O)ORₐ, —PO₃Rₐ, —PO(ORₐ)(OR_{b}), —SO₂Rₐ, —S(O)Rₐ, or —SO(N)Rₐ, wherein Rₐ and R_{b} are the same or different and each independently C₁₋₆alkyl, cycloalkyl, or heterocycloalkyl,
R₂ is hydrogen, C₁₋₆alkyl, halo-C₁₋₆alkyl, aryl, heteroaryl, or cycloalkyl, wherein the alkyl, alkoxy, aryl, heteroaryl or cycloalkyl is unsbstituted or substituted with at least one selected from C₁₋₆alkyl, halo-C₁₋₆alkyl, aryl, halogen, C₁₋₆alkoxy, CN, or NO₂, and
R₃ is C₁₋₅alkyl, phenyl, pididyl, cyclohexyl, or piperidyl, wherein R₃ is unsbstituted or substituted with at least one selected from C₁₋₆alkyl, halogen, halo-C₁₋₆alkyl, C₁₋₆alkoxy, CN, NO₂, —N(C₁₋₃alkyl)(C₁₋₃alkyl), aryl, heterocycloalkyl, or heterocycloalkyl substituted with methyl.

3. The compound of claim 2 or a pharmaceutically acceptable salt thereof, wherein
X is N or CH,
Y is O, NH or —N(C₁₋₆alkyl)-,
Z is —NH—,
R₁ is -A-(B)-R₄, wherein A is imidazole, pyrazole, triazole, pyridine, piperidine, piperazine, or tetrahydropyridine; B is a direct bond, or piperidine, piperazine, azetidine, or pyrrolidine; R₄ is hydrogen, hydrogen, —C(O)—C₁₋₆alkyl, or —C(O)O—C₁₋₆alkyl,
R₂ is C₁₋₆alkyl, halo-C₁₋₆alkyl, or phenyl, wherein the alkyl or phenyl is unsbstituted or substituted with at least one selected from C₁₋₆alkyl, halo-C₁₋₆alkyl, halogen, C₁₋₆alkoxy, CN, or NO₂, and
R₃ is phenyl, pididyl, cyclohexyl, or piperidyl, wherein the phenyl, pididyl, cyclohexyl, or piperidyl is unsubstituted or substituted with at least one selected from C₁₋₆alkyl, halogen, or C₁₋₆alkoxy.

4. The compound of claim 3 or a pharmaceutically acceptable salt thereof, wherein
X is N or CH (preferably, N),
Y is O or NH (preferably, O),
Z is —NH—,
R₁ is -A-(B)-R₄, wherein A is pyrazole, triazole, pyridine, or tetrahydropyridine; B is a direct bond, or piperidine, piperazine, azetidine, or pyrrolidine; R₄ is hydrogen, —C(O)—C₁₋₃alkyl, or —C(O)O—C₁₋₆alkyl,
R₂ is C₁₋₆alkyl, halo-C₁₋₆alkyl, or phenyl, wherein the alkyl or phenyl is unsbstituted or substituted with at least one selected from C₁₋₆alkyl, halo-C₁₋₆alkyl, halogen, or C₁₋₆alkoxy, and
R₃ is phenyl, pididyl, cyclohexyl, or piperidyl, wherein the phenyl, pididyl, cyclohexyl, or piperidyl is unsubstituted or substituted with at least one selected from C₁₋₆alkyl, halogen, or C₁₋₆alkoxy.

5. The compound of claim 1 or a pharmaceutically acceptable salt thereof, wherein the compound is any one of the following compounds:

| Compound | IUPAC Name |
|---|---|
| TDH-001 | (R)-2-((4-fluorophenyl)amino)-2-oxo-1-phenylethyl 3-aminopyrazine-2-carboxylate |
| TDH-002 | (S)-2-((4-fluorophenyl)amino)-2-oxo-1-phenylethyl 3-aminopyrazine-2-carboxylate |
| TDH-066 | (R)-1′-(tert-butyl) 5-(2-((4-fluorophenyl)amino)-2-oxo-1-phenylethyl) 6-amino-3′,6′-dihydro- |
| | [3,4′-bipyridine]-1′, 5(2′H)-dicarboxylate |
| TDH-067 | (S)-3-amino-6-bromo-N-(2-((4-fluorophenyl)amino)-2-oxo-1-phenylethyl)pyrazine-2- |
| | carboxamide |
| TDH-068 | (R)-2-((4-fluorophenyl)amino)-2-oxo-1-phenylethyl 3-amino-6-(1,2,3,6-tetrahydropyridin-4- |
| | yl)pyrazine-2-carboxylate hydrochloride |
| TDH-069 | (R)-2-((4-fluorophenyl)amino)-2-oxo-1-phenylethyl 3-amino-6-(1-(piperidin-4-yl)-1H- |
| | pyrazol-4-yl)pyrazine-2-carboxylate hydrochloride |
| TDH-070 | (R)-2-((4-fluorophenyl)amino)-2-oxo-1-phenylethyl 6-amino-1′,2′,3′,6′-tetrahydro-[3,4′- |
| | bipyridine]-5-carboxylate hydrochloride |
| TDH-071 | (S)-3-amino-N-(2-((4-fluorophenyl)amino)-2-oxo-1-phenylethyl)pyrazine-2-carboxamide |
| TDH-097 | (R)-2-((4-fluorophenyl)amino)-2-oxo-1-phenylethyl 6-(1-(1-acetylpiperidin-4-yl)-1H-pyrazol- |
| | 4-yl)-3-aminopyrazine-2-carboxylate |
| TDH-098 | tert-butyl (R)-4-(4-(5-amino-6-((2-((4-fluorophenyl)amino)-2-oxo-1- |
| | phenylethyl)carbamoyl)pyrazin-2-yl)-1H-pyrazol-1-yl)piperidine-1-carboxylate |
| TDH-099 | (R)-3-amino-N-(2-((4-fluorophenyl)amino)-2-oxo-1-phenylethyl)-6-(1-(piperidin-4-yl)-1H- |
| | pyrazol-4-yl)pyrazine-2-carboxamide hydrochloride |
| TDH-100 | (S)-2-((4-fluorophenyl)amino)-2-oxo-1-phenylethyl 3-amino-6-(1-(1-(2,2-dimethyl-4-oxo- |
| | 3,8,11-trioxa-5-azatridecan-13-yl)piperidin-4-yl)-1H-pyrazol-4-yl)pyrazine-2-carboxylate |
| TDH-106 | (R)-3-amino-N-(2-((4-fluorophenyl)amino)-2-oxo-1-phenylethyl)pyrazine-2-carboxamide |
| TDH-112 | (R)-2-((4-fluorophenyl)amino)-2-oxo-1-phenylethyl 2-amino-5-(1-(piperidin-4-yl)-1H- |
| | pyrazol-4-yl)nicotinate hydrochloride |
| TDH-180 | (R)-2-((4-fluorophenyl)amino)-2-oxo-1-phenylethyl 3-amino-6-(1-(1-(tert- |
| | butoxycarbonyl)piperidin-4-yl)-1H-1,2,3-triazol-4-yl)pyrazine-2-carboxylate |
| TDH-181 | (R)-2-((4-fluorophenyl)amino)-2-oxo-1-phenylethyl 3-amino-6-(1-(piperidin-4-yl)-1H-1,2,3- |
| | triazol-4-yl)pyrazine-2-carboxylate |
| TDH-182 | (R)-2-((4-fluorophenyl)amino)-2-oxo-1-phenylethyl 3-amino-6-(1-(1-methylpiperidin-4-yl)- |
| | 1H-1,2,3-triazol-4-yl)pyrazine-2-carboxylate |
| TDH-183 | (R)-2-((4-fluorophenyl)amino)-2-oxo-1-phenylethyl 3-amino-6-(6-(4-ethylpiperazin-1- |
| | yl)pyridin-3-yl)pyrazine-2-carboxylate |
| TDH-184 | (R)-2-((4-fluorophenyl)amino)-2-oxo-1-phenylethyl 6-(6-(4-acetylpiperazin-1-yl)pyridin-3- |
| | yl)-3-aminopyrazine-2-carboxylate |
| TDH-185 | (R)-2-((4-fluorophenyl)amino)-2-oxo-1-phenylethyl 6-(1-(1-acetylazetidin-3-yl)-1H-pyrazol- |
| | 4-yl)-3-aminopyrazine-2-carboxylate |
| TDH-227 | 2-((4-fluorophenyl)amino)-2-oxo-1-phenylethyl 3-amino-6-(1-((S)-1-(tert- |
| | butoxycarbonyl)pyrrolidin-3-yl)-1H-pyrazol-4-yl)pyrazine-2-carboxylate |
| TDH-228 | 2-((4-fluorophenyl)amino)-2-oxo-1-phenylethyl 6-(1-((S)-1-acetylpyrrolidin-3-yl)-1H- |
| | pyrazol-4-yl)-3-aminopyrazine-2-carboxylate |
| TDH-229 | 2-((4-fluorophenyl)amino)-2-oxo-1-phenylethyl 6-(1-((R)-1-acetylpyrrolidin-3-yl)-1H- |
| | pyrazol-4-yl)-3-aminopyrazine-2-carboxylate |
| TDH-313 | (R)-2-((4-fluorophenyl)amino)-2-oxo-1-phenylethyl 3-amino-6-bromopyrazine-2- |
| | carboxylate |
| TDH-314 | (R)-2-((3-chlorophenyl)amino)-2-oxo-1-phenylethyl 3-amino-6-(1-(piperidin-4-yl)-1H- |
| | pyrazol-4-yl)pyrazine-2-carboxylate hydrochloride |
| TDH-315 | (R)-2-((4-chlorophenyl)amino)-2-oxo-1-phenylethyl 3-amino-6-(1-(piperidin-4-yl)-1H- |
| | pyrazol-4-yl)pyrazine-2-carboxylate hydrochloride |
| TDH-316 | (R)-2-((3-methoxyphenyl)amino)-2-oxo-1-phenylethyl 3-amino-6-(1-(piperidin-4-yl)-1H- |
| | pyrazol-4-yl)pyrazine-2-carboxylate hydrochloride |
| TDH-317 | (R)-2-((4-methoxyphenyl)amino)-2-oxo-1-phenylethyl 3-amino-6-(1-(piperidin-4-yl)-1H- |
| | pyrazol-4-yl)pyrazine-2-carboxylate hydrochloride |
| TDH-318 | (R)-2-oxo-1-phenyl-2-(m-tolylamino)ethyl 3-amino-6-(1-(piperidin-4-yl)-1H-pyrazol-4- |
| | yl)pyrazine-2-carboxylate hydrochloride |
| TDH-319 | (R)-2-oxo-1-phenyl-2-(p-tolylamino)ethyl 3-amino-6-(1-(piperidin-4-yl)-1H-pyrazol-4- |
| | yl)pyrazine-2-carboxylate hydrochloride |
| TDH-320 | (R)-2-((4-fluorophenyl)amino)-2-oxo-1-(p-tolyl)ethyl 3-amino-6-(1-(piperidin-4-yl)-1H- |
| | pyrazol-4-yl)pyrazine-2-carboxylate hydrochloride |
| TDH-321 | (R)-2-((4-fluorophenyl)amino)-2-oxo-1-(p-tolyl)ethyl 3-amino-6-(1-(piperidin-4-yl)-1H- |
| | pyrazol-4-yl)pyrazine-2-carboxylate hydrochloride |
| TDH-322 | (R)-1-(4-chlorophenyl)-2-((4-fluorophenyl)amino)-2-oxoethyl 3-amino-6-(1-(piperidin-4-yl)- |
| | 1H-pyrazol-4-yl)pyrazine-2-carboxylate hydrochloride |
| TDH-323 | (R)-2-((4-fluorophenyl)amino)-2-oxo-1-(p-tolyl)ethyl 6-(1-(1-acetylpiperidin-4-yl)-1H- |
| | pyrazol-4-yl)-3-aminopyrazine-2-carboxylate |
| TDH-324 | (R)-1-(3-chlorophenyl)-2-((4-fluorophenyl)amino)-2-oxoethyl 6-(1-(1-acetylpiperidin-4-yl)- |
| | 1H-pyrazol-4-yl)-3-aminopyrazine-2-carboxylate |
| TDH-325 | (R)-1-(4-chlorophenyl)-2-((4-fluorophenyl)amino)-2-oxoethyl 6-(1-(1-acetylpiperidin-4-yl)- |
| | 1H-pyrazol-4-yl)-3-aminopyrazine-2-carboxylate |
| TDH-326 | (R)-2-((4-fluorophenyl)amino)-1-(4-methoxyphenyl)-2-oxoethyl 3-amino-6-(1-(piperidin-4- |
| | yl)-1H-pyrazol-4-yl)pyrazine-2-carboxylate hydrochloride |
| TDH-327 | (R)-2-((4-fluorophenyl)amino)-2-oxo-1-phenylethyl 3-amino-6-(4-chlorophenyl)pyrazine-2- |
| | carboxylate |
| TDH-329 | (R)-2-((4-fluorophenyl)amino)-2-oxo-1-phenylethyl 3-amino-6-(4- |
| | (methylsulfonyl)phenyl)pyrazine-2-carboxylate |
| TDH-335 | (R)-2-((4-fluorophenyl)amino)-2-oxo-1-phenylethyl 3-amino-6-(pyridin-4-yl)pyrazine-2- |
| | carboxylate |
| TDH-424 | (R)-2-((4-methylcyclohexyl)amino)-2-oxo-1-phenylethyl 3-amino-6-(1-(piperidin-4-yl)-1H- |
| | pyrazol-4-yl)pyrazine-2-carboxylate hydrochloride |
| TDH-444 | (R)-2-((1-methylpiperidin-4-yl)amino)-2-oxo-1-phenylethyl 3-amino-6-(1-(piperidin-4-yl)- |
| | 1H-pyrazol-4-yl)pyrazine-2-carboxylate hydrochloride |
| TDH-455 | (R)-2-(4-methylpiperazin-1-yl)-2-oxo-1-phenylethyl 3-amino-6-(1-(piperidin-4-yl)-1H- |
| | pyrazol-4-yl)pyrazine-2-carboxylate hydrochloride |
| TDH-456 | (R)-2-((3-fluorophenyl)amino)-2-oxo-1-phenylethyl 3-amino-6-(1-(piperidin-4-yl)-1H- |
| | pyrazol-4-yl)pyrazine-2-carboxylate hydrochloride |
| TDH-457 | (R)-2-((4-fluorobenzyl)amino)-2-oxo-1-phenylethyl 3-amino-6-(1-(piperidin-4-yl)-1H- |
| | pyrazol-4-yl)pyrazine-2-carboxylate hydrochloride |
| TDH-458 | (R)-2-((2-fluorophenyl)amino)-2-oxo-1-phenylethyl 3-amino-6-(1-(piperidin-4-yl)-1H- |
| | pyrazol-4-yl)pyrazine-2-carboxylate hydrochloride |
| TDH-459 | (R)-2-oxo-1-phenyl-2-((4-(trifluoromethyl)phenyl)amino)ethyl 3-amino-6-(1-(piperidin-4-yl)- |
| | 1H-pyrazol-4-yl)pyrazine-2-carboxylate hydrochloride |
| TDH-460 | (R)-2-oxo-1-phenyl-2-((3-(trifluoromethyl)phenyl)amino)ethyl 3-amino-6-(1-(piperidin-4-yl)- |
| | 1H-pyrazol-4-yl)pyrazine-2-carboxylate hydrochloride |
| TDH-461 | (R)-2-((4-(4-methylpiperazin-1-yl)phenyl)amino)-2-oxo-1-phenylethyl 3-amino-6-(1- |
| | (piperidin-4-yl)-1H-pyrazol-4-yl)pyrazine-2-carboxylate hydrochloride |
| TDH-462 | (R)-2-((3-(4-methylpiperazin-1-yl)phenyl)amino)-2-oxo-1-phenylethyl 3-amino-6-(1- |
| | (piperidin-4-yl)-1H-pyrazol-4-yl)pyrazine-2-carboxylate hydrochloride |
| TDH-463 | (R)-2-oxo-1-phenyl-2-(phenylamino)ethyl 3-amino-6-(1-(piperidin-4-yl)-1H-pyrazol-4- |
| | yl)pyrazine-2-carboxylate hydrochloride |
| TDH-464 | (R)-2-((4-fluoro-3-methylphenyl)amino)-2-oxo-1-phenylethyl 3-amino-6-(1-(piperidin-4-yl)- |
| | 1H-pyrazol-4-yl)pyrazine-2-carboxylate hydrochloride |
| TDH-465 | (R)-1-(2-chlorophenyl)-2-((4-fluorophenyl)amino)-2-oxoethyl 3-amino-6-(1-(piperidin-4-yl)- |
| | 1H-pyrazol-4-yl)pyrazine-2-carboxylate hydrochloride |
| TDH-468 | (R)-2-oxo-1-phenyl-2-(pyridin-3-ylamino)ethyl 3-amino-6-(1-(piperidin-4-yl)-1H-pyrazol-4- |
| | yl)pyrazine-2-carboxylate hydrochloride |
| TDH-469 | (R)-2-((3-(dimethylamino)phenyl)amino)-2-oxo-1-phenylethyl 3-amino-6-(1-(piperidin-4-yl)- |
| | 1H-pyrazol-4-yl)pyrazine-2-carboxylate hydrochloride |
| TDH-470 | (R)-2-((4-(dimethylamino)phenyl)amino)-2-oxo-1-phenylethyl 3-amino-6-(1-(piperidin-4-yl)- |
| | 1H-pyrazol-4-yl)pyrazine-2-carboxylate hydrochloride |
| TDH-471 | (R)-1-((4-fluorophenyl)amino)-3-methyl-1-oxobutan-2-yl 3-amino-6-(1-(piperidin-4-yl)-1H- |
| | pyrazol-4-yl)pyrazine-2-carboxylate hydrochloride |
| TDH-479 | (R)-2-oxo-1-phenyl-2-(pyridin-4-ylamino)ethyl 3-amino-6-(1-(piperidin-4-yl)-1H-pyrazol-4- |
| | yl)pyrazine-2-carboxylate--2,2,2-trifluoroacetaldehyde |
| TDH-488 | (R)-2-((3,4-difluorophenyl)amino)-2-oxo-1-phenylethyl 3-amino-6-(1-(piperidin-4-yl)-1H- |
| | pyrazol-4-yl)pyrazine-2-carboxylate hydrochloride |
| TDH-489 | 1-(4-fluorophenyl)-2-((4-fluorophenyl)amino)-2-oxoethyl 3-amino-6-(1-(piperidin-4-yl)-1H- |
| | pyrazol-4-yl)pyrazine-2-carboxylate hydrochloride |
| TDH-490 | (R)-3-amino-N-(2-((4-fluorophenyl)amino)-2-oxo-1-phenylethyl)-N-methyl-6-(1-(piperidin-4- |
| | yl)-1H-pyrazol-4-yl)pyrazine-2-carboxamide hydrochloride |

6. A method for preparing a compound that inhibits or degrades MLKL using the compound of claim 1.

7. A compound of Chemical Formula 2: 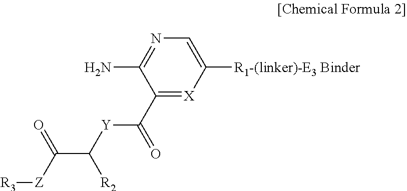
or a pharmaceutically acceptable salt thereof,
in the Chemical Formula 2,
X, Y, Z, R₂ and R₃ are the same as X, Y, Z, R₂ and R₃ of Chemical Formula 1 of claim 1,
R₁ is —(CH₂)ₙ—, aryl, heteroaryl, cycloalkyl, heterocycloalkyl, or -A-(B)-, wherein n is an integer of from 1 to 10,
A is a 4-6-membered heteroaryl or heterocycloalkyl containing one or more N,
B is a direct bond (i.e. does not exist), or a 4-7-membered heterocycloalkyl containing one or more N,
the linker may not exist, and if it exists, it is a connecting group that connects R₁ and E3 Binder, and
E3 Binder is an E3 ligase ligand.

8. The compound of claim 7 or a pharmaceutically acceptable salt thereof, wherein
X is N or CH,
Y is S, O, NH or —N(C₁₋₆alkyl)-,
Z is a direct bond, —NH—, —N—(C₁₋₄alkyl)-, or 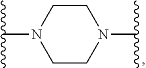 [SMILES: *N1CCN(*)CC1]
R₂ is hydrogen, C₁₋₆alkyl, halo-C₁₋₆alkyl, aryl, heteroaryl, or cycloalkyl, wherein the alkyl, alkoxy, aryl, heteroaryl or cycloalkyl is unsbstituted or substituted with at least one selected from C₁₋₆alkyl, halo-C₁₋₆alkyl, aryl, halogen, C₁₋₆alkoxy, CN, or NO₂,
R₃ is C₁₋₅alkyl, phenyl, pididyl, cyclohexyl, or piperidyl, wherein R₃ is unsbstituted or substituted with at least one selected from C₁₋₆alkyl, halogen, halo-C₁₋₆alkyl, C₁₋₆alkoxy, CN, NO₂, or aryl, and
R₁ is aryl, heteroaryl, or -A-(B)-, wherein A is a 4-6-membered heteroaryl or heterocycloalkyl containing one or more N; and B is a direct bond, or a 4-7-membered heterocycloalkyl containing one or more N.

9. The compound of claim 8 or a pharmaceutically acceptable salt thereof, wherein
X is N or CH,
Y is O, NH or —N(C₁₋₆alkyl)-,
Z is —NH—,
R₂ is C₁₋₆alkyl, halo-C₁₋₆alkyl, or phenyl, wherein the alkyl or phenyl is unsbstituted or substituted with at least one selected from C₁₋₆alkyl, halo-C₁₋₆alkyl, halogen, C₁₋₆alkoxy, CN, or NO₂,
R₃ is phenyl, pididyl, cyclohexyl, or piperidyl, wherein the phenyl, pididyl, cyclohexyl, or piperidyl is unsbstituted or substituted with at least one selected from C₁₋₆alkyl, halogen, or C₁₋₆alkoxy, and
R₁ is -A-(B)-, wherein A is imidazole, pyrazole, triazole, pyridine, piperidine, piperazine, or tetrahydropyridine; B is a direct bond, or piperidine, piperazine, azetidine, or pyrrolidine.

10. The compound of claim 9 or a pharmaceutically acceptable salt thereof, wherein
X is N or CH (preferably, N),
Y is O or NH (preferably, O),
Z is —NH—,
R₂ is C₁₋₆alkyl, halo-C₁₋₆alkyl, or phenyl, wherein the alkyl or phenyl is unsbstituted or substituted with at least one selected from C₁₋₆alkyl, halo-C₁₋₆alkyl, halogen, or C₁₋₆alkoxy,
R₃ is phenyl, pididyl, cyclohexyl, or piperidyl, wherein the phenyl, pididyl, cyclohexyl, or piperidyl is unsbstituted or substituted with at least one selected from C₁₋₆alkyl, halogen, or C₁₋₆alkoxy, and
R₁ is -A-(B)-, wherein A is imidazole, pyrazole, triazole, pyridine, or tetrahydropyridine; B is a direct bond, or piperidine, piperazine, azetidine, or pyrrolidine.

11. The compound of claim 7 or a pharmaceutically acceptable salt thereof, wherein
the linker is a direct bond or have a structure of Chemical Formula 6: 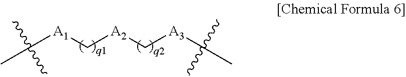
in the Chemical Formula 6,
A₁ is a direct bond, —C(O)—, —C(O)NH—, or —S(O)₂—,
A₂ is a direct bond, O, —C(O)—, —CH(OH)—, —N(C₁₋₃alkyl)-, —(OCH₂CH₂)_{q3}—, C₃₋₈cycloalkyl, heterocycle, or heteroaryl (preferably, imidazole, triazole, piperidine, piperazine, pyrrolidine, azetidine, or 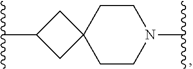 [SMILES: *C1CC2(CCN(*)CC2)C1]
more preferably cyclohexane, imidazole, or triazole),
A₃ is a direct bond, CO, NH, CC, heterocycloalkyl having bi-cyclic structure (preferably, 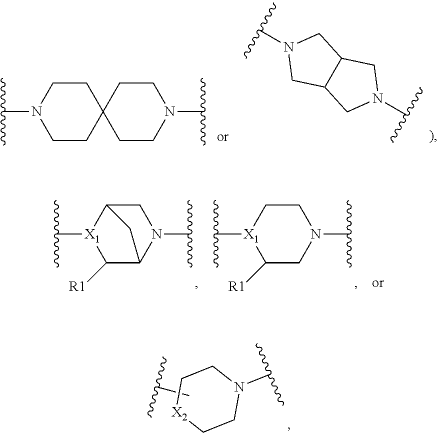
wherein, X₁ is N, CH or C(OH), X₂ is O or CH, and R₁ is hydrogen, —C₁₋₃alkyl, or —C₁₋₃alkyl-OH, and
q1, q2 and q3 are each independently an integer of from 0 to 12 (preferably, q3 is an integer of from 0 to 5).

12. The compound of claim 11 or a pharmaceutically acceptable salt thereof, wherein the linker is any one of the followings:

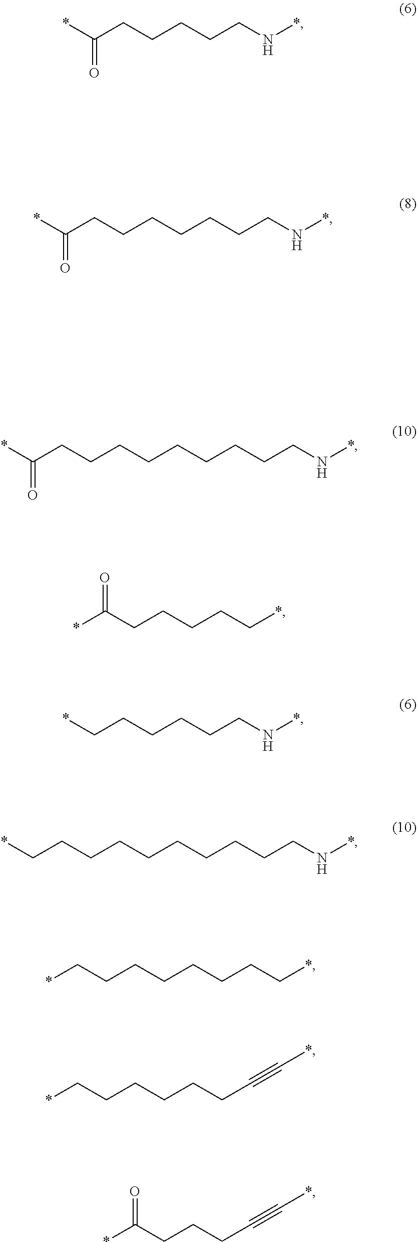

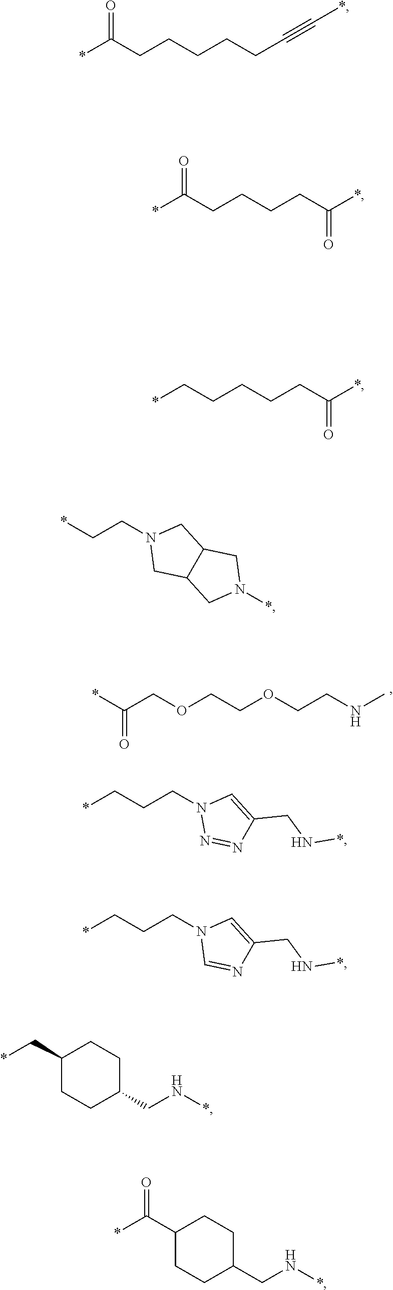

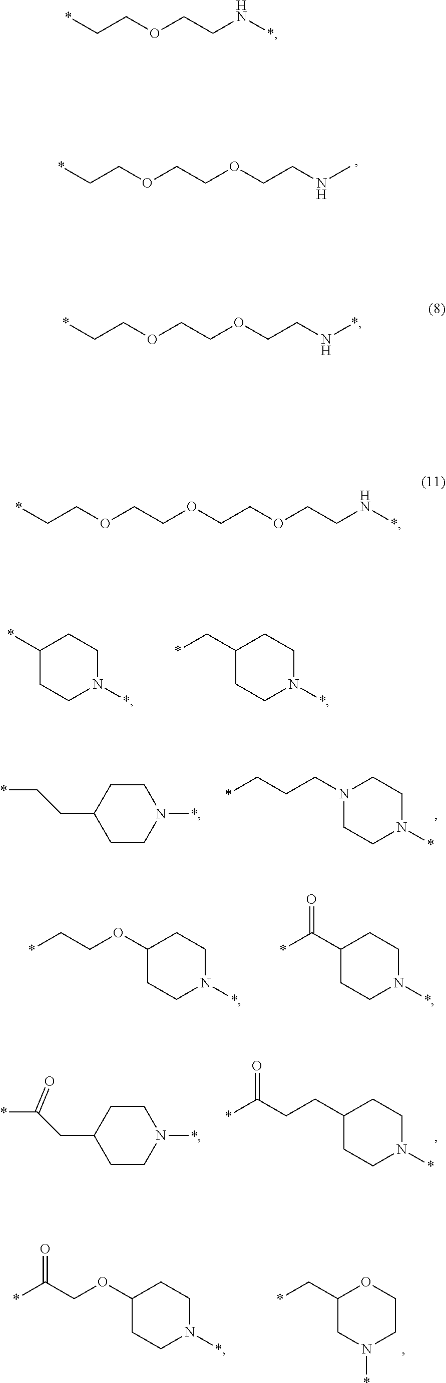

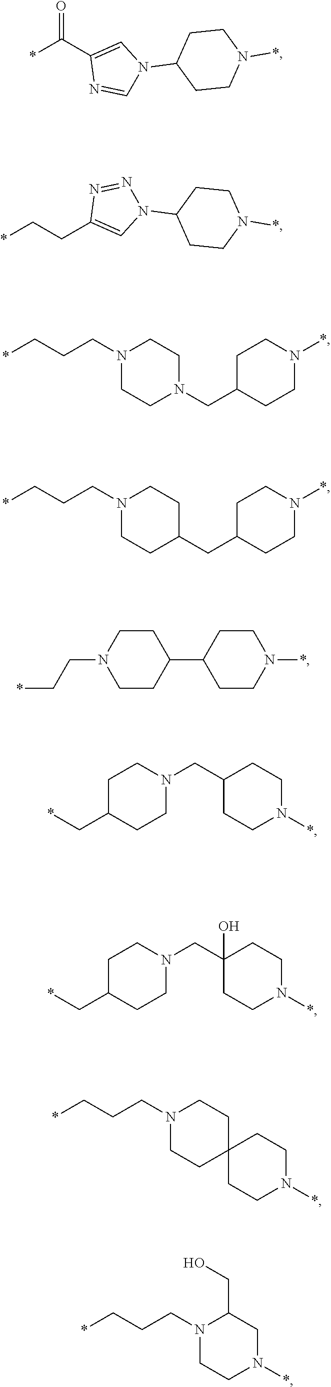

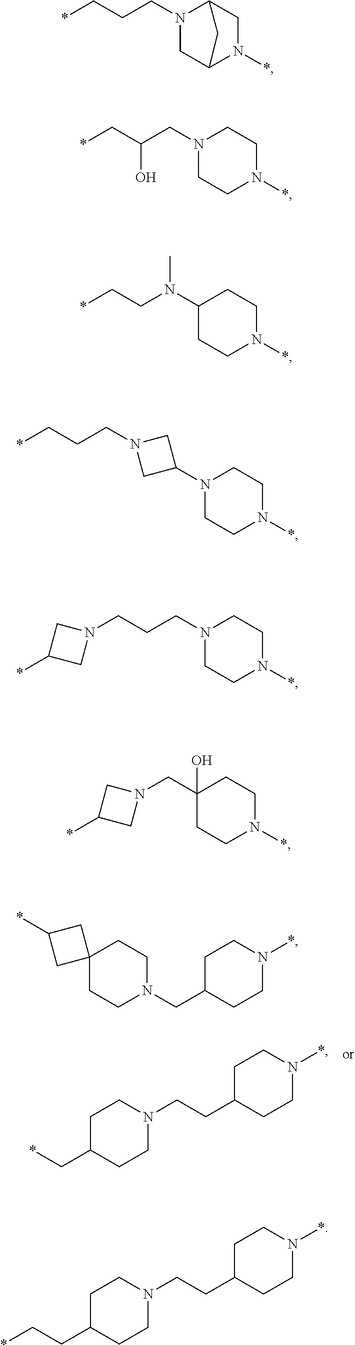

13. The compound of claim 7 or a pharmaceutically acceptable salt thereof, wherein the E3 Binder is *-(Chemical Formula X)-Chemical Formula Y,
wherein Chemical Formula X does not exist or is a moiety connecting Chemical Formula Y and the rest of Chemical Formula 2, and
Chemical Formula Y is CRBN E3 Ubiquitin Ligase binding moiety, VHL E3 Ubiquitin Ligase binding moiety, or IAP E3 Ubiquitin Ligase binding moiety.

14. The compound of claim 13 or a pharmaceutically acceptable salt thereof, wherein Chemical Formula Y is a compound of any one of Chemical Formulas 3 to 5: 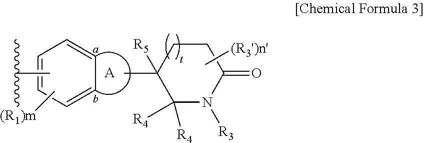
in the Chemical Formula 3,
the 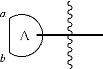
is any one of the following: 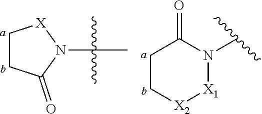
X is C(O) or C(R₃)₂;
X₁-X₂ is N═N, C(R₃)═N or C(R₃)₂—C(R₃)₂;
each R₁ is each independently halogen, nitro, NH₂, OH, C(O)OH, C₁-C₆ alkyl, or C₁-C₆ alkoxy;
each R₃ is each independently H or C₁-C₃ alkyl optionally substituted with C₆-C₁₀ aryl or 5- to 10-membered heteroaryl;
each R₃′ is each independently C₁-C₃ alkyl;
each R₄ is each independently H or C₁-C₃ alkyl; or two R₄, together with the carbon atom to which they are attached, form C(O), a C₃-C₆ cycloalkyl, or a 4-, 5-, or 6-membered heterocycle containing 1 or 2 heteroatoms selected from N and O;
R₅ is D (deuterium), H, C₁-C₃alkyl, F, or Cl;
t is 0, 1 or 2;
m is 0, 1, 2 or 3; and
n and n′ are each independently 0, 1 or 2, 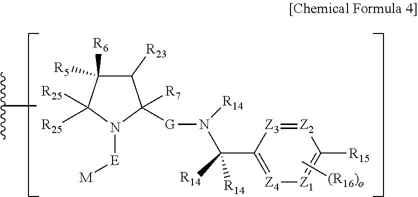
in the Chemical Formula 4,
each R₅ and R₆ is each independently OH, SH, or optionally substituted alkyl, or R₅, R₆, and the carbon atom to which they are attached form a carbonyl;
R₇ is H or optionally substituted alkyl;
E is a direct bond, C═O, or C═S;
G is a direct bond, optionally substituted alkyl, —COOH, or CO, or C═N—R₈, wherein R₈ is H, CN, optionally substituted alkyl, or optionally substituted alkoxy;
M is optionally substituted aryl, optionally substituted heteroaryl, optionally substituted heterocycle or 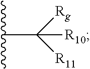
each R₉ and R₁₀ are each independently H, optionally substituted alkyl, optionally substituted cycloalkyl, optionally substituted hydroxyalkyl, optionally substituted thioalkyl, disulphide connected to linker or X, optionally substituted heteroaryl, or haloalkyl; or R₉, R₁₀, and the carbon atom to which they are attached form an optionally substituted cycloalkyl;
R₁₁ is optionally substituted heterocycle, optionally substituted alkoxy, optionally substituted heteroaryl, optionally substituted aryl, or 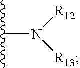
R₁₂ is H or optionally substituted alkyl;
R₁₃ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate,
each R₁₄ is each independently H, haloalkyl, optionally substituted cycloalkyl, optionally substituted alkyl or optionally substituted heterocycloalkyl;
R₁₅ is H, optionally substituted heteroaryl, haloalkyl, optionally substituted aryl, optionally substituted alkoxy, or optionally substituted heterocycle;
each R₁₆ is each independently halogen, optionally substituted alkyl, optionally substituted haloalkyl, CN, or optionally substituted haloalkoxy;
each R₂₅ is each independently H or optionally substituted alkyl; or both R₂₅ groups can be taken together to form an oxo or optionally substituted cycloalkyl group;
R₂₃ is H or OH;
Z₁, Z₂, Z₃, and Z₄ are each independently C or N; and
o is 0, 1, 2, 3, or 4, 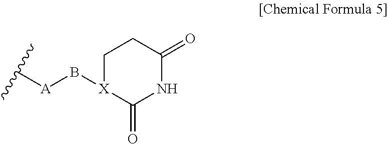
in the Chemical Formula 5,
X is CH or N,
A is C₁₋₃alkyl, aryl, heteroaryl, or heterocycloalkyl, wherein the aryl, heteroaryl, or heterocycloalkyl is unsubstituted or one or more hydrogen of the aryl, heteroaryl, or heterocycloalkyl is substituted with at least one selected from the group consisting of ═O, C₁₋₃alkoxy, C₁₋₃alkyl, halogen, nitrile, nitro, di-C₁₋₂alkylamine, —COOH, and —COO—C₁₋₂alkyl, and
B is a direct bond or —C(O)NH—.

15. The compound of claim 14 or a pharmaceutically acceptable salt thereof wherein Chemical Formula 3 has one of the following structures:

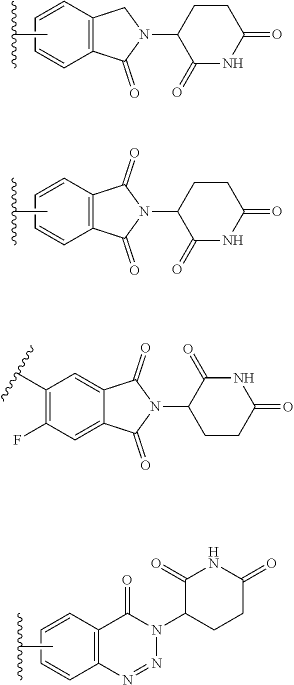

16. The compound of claim 14 or a pharmaceutically acceptable salt thereof, wherein Chemical Formula 4 has the following structure:

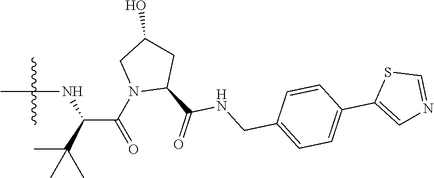

17. The compound of claim 14 or a pharmaceutically acceptable salt thereof, wherein Chemical Formula 5 has one of the following structures:

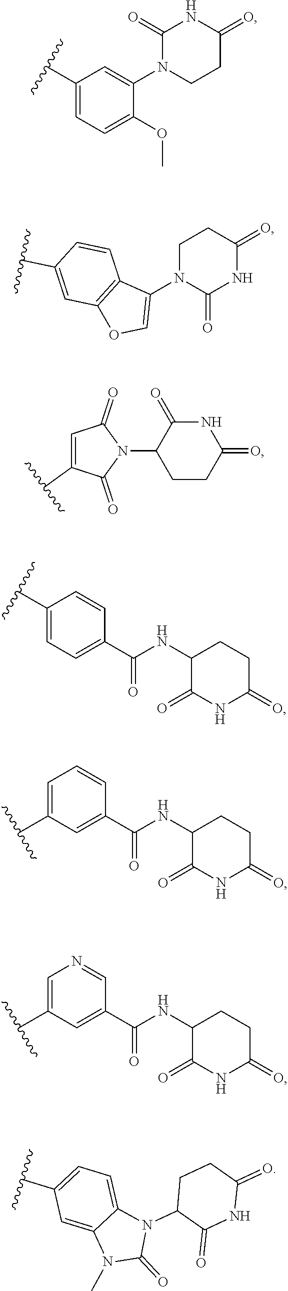

18. The compound of claim 13 or a pharmaceutically acceptable salt thereof, wherein Chemical Formula X is Chemical Formula 7: 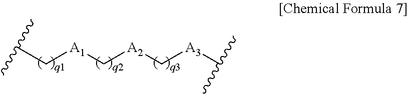
in the Chemical Formula 7,
A₁ is a direct bond, CO, or —C(O)NH—,
A₂ and A₃ are each independently a direct bond, O, CO, NH, CC, phenyl, heterocycloalkyl (preferably, piperidine, piperazine, pyrrolidine, or azetidine), and
q1, q2 and q3 are each independently an integer of from 0 to 3.

19. The compound of claim 18 or a pharmaceutically acceptable salt thereof, wherein Chemical Formula X is one of the following structures:

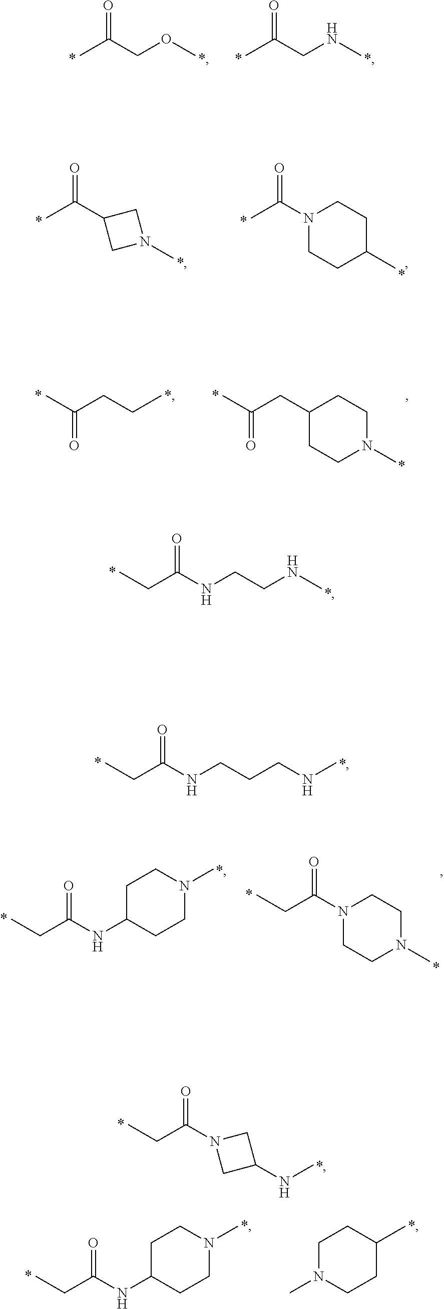

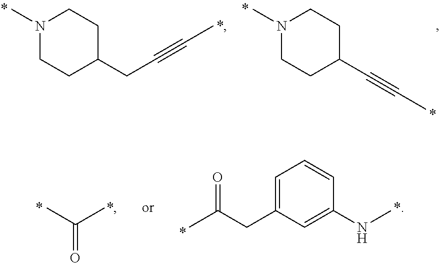

20. The compound of claim 7 or a pharmaceutically acceptable salt thereof, wherein the compound is any one of the following compounds:

| Compound | IUPAC Name |
|---|---|
| TDH-072 | (R)-2-((4-fluorophenyl)amino)-2-oxo-1-phenylethyl 6-amino-1′-(6-(2-((2-(2,6- |
| | dioxopiperidin-3-yl)-1,3-dioxoisoindolin-5-yl)amino)acetamido)hexanoyl)-1′,2′,3′,6′- |
| | tetrahydro-[3,4′-bipyridine]-5-carboxylate |
| TDH-073 | (R)-2-((4-fluorophenyl)amino)-2-oxo-1-phenylethyl 6-amino-1′-(6-((2-(2,6-dioxopiperidin-3- |
| | yl)-1,3-dioxoisoindolin-4-yl)amino)hexanoyl)-1′,2′,3′,6′-tetrahydro-[3,4′-bipyridine]-5- |
| | carboxylate |
| TDH-074 | (R)-2-((4-fluorophenyl)amino)-2-oxo-1-phenylethyl 6-amino-1′-(6-(1-(2-(2,6-dioxopiperidin- |
| | 3-yl)-1,3-dioxoisoindolin-5-yl)piperidine-4-carboxamido)hexanoyl)-1′,2′,3′,6′-tetrahydro- |
| | [3,4′-bipyridine]-5-carboxylate |
| TDH-075 | (R)-2-((4-fluorophenyl)amino)-2-oxo-1-phenylethyl 6-amino-1′-(6-((2-(2,6-dioxopiperidin-3- |
| | yl)-1,3-dioxoisoindolin-5-yl)amino)hexanoyl)-1′,2′,3′,6′-tetrahydro-[3,4′-bipyridine]-5- |
| | carboxylate |
| TDH-078 | (R)-2-((4-fluorophenyl)amino)-2-oxo-1-phenylethyl 6-amino-1′-(6-(1-(2-(2,6-dioxopiperidin- |
| | 3-yl)-1,3-dioxoisoindolin-4-yl)azetidine-3-carboxamido)hexanoyl)-1′,2′,3′,6′-tetrahydro- |
| | [3,4′-bipyridine]-5-carboxylate |
| TDH-079 | (R)-2-((4-fluorophenyl)amino)-2-oxo-1-phenylethyl 6-amino-1′-(6-(2-((2-(2,6- |
| | dioxopiperidin-3-yl)-1,3-dioxoisoindolin-4-yl)amino)acetamido)hexanoyl)-1′,2′,3′,6′- |
| | tetrahydro-[3,4′-bipyridine]-5-carboxylate |
| TDH-080 | (R)-2-((4-fluorophenyl)amino)-2-oxo-1-phenylethyl 6-amino-1′-(6-(1-(2-(2,6-dioxopiperidin- |
| | 3-yl)-1,3-dioxoisoindolin-4-yl)piperidine-4-carboxamido)hexanoyl)-1′,2′,3′,6′-tetrahydro- |
| | [3,4′-bipyridine]-5-carboxylate |
| TDH-081 | (R)-2-((4-fluorophenyl)amino)-2-oxo-1-phenylethyl 6-amino-1′-(6-(2-((2-(2,6- |
| | dioxopiperidin-3-yl)-1,3-dioxoisoindolin-4-yl)oxy)acetamido)hexanoyl)-1′,2′,3′,6′-tetrahydro- |
| | [3,4′-bipyridine]-5-carboxylate |
| TDH-082 | (R)-2-((4-fluorophenyl)amino)-2-oxo-1-phenylethyl 6-amino-1′-(2-(2-(2-((2-(2,6- |
| | dioxopiperidin-3-yl)-1,3-dioxoisoindolin-4-yl)amino)ethoxy)ethoxy)acetyl)-1′,2′,3′,6′- |
| | tetrahydro-[3,4′-bipyridine]-5-carboxylate |
| TDH-083 | (R)-2-((4-fluorophenyl)amino)-2-oxo-1-phenylethyl 6-amino-1′-(2-(2-(2-((2-(2,6- |
| | dioxopiperidin-3-yl)-1,3-dioxoisoindolin-5-yl)amino)ethoxy)ethoxy)acetyl)-1′,2′,3′,6′- |
| | tetrahydro-[3,4′-bipyridine]-5-carboxylate |
| TDH-084 | (R)-2-((4-fluorophenyl)amino)-2-oxo-1-phenylethyl 6-amino-1′-(2-(2-(2-(2-((2-(2,6- |
| | dioxopiperidin-3-yl)-1,3-dioxoisoindolin-4-yl)amino)acetamido)ethoxy)ethoxy)acetyl)- |
| | 1′,2′,3′,6′-tetrahydro-[3,4′-bipyridine]-5-carboxylate |
| TDH-085 | (R)-2-((4-fluorophenyl)amino)-2-oxo-1-phenylethyl 6-amino-1′-(2-(2-(2-(2-((2-(2,6- |
| | dioxopiperidin-3-yl)-1,3-dioxoisoindolin-5-yl)amino)acetamido)ethoxy)ethoxy)acetyl)- |
| | 1′,2′,3′,6′-tetrahydro-[3,4′-bipyridine]-5-carboxylate |
| TDH-086 | (R)-2-((4-fluorophenyl)amino)-2-oxo-1-phenylethyl 6-amino-1′-(2-(2-(2-(1-(2-(2,6- |
| | dioxopiperidin-3-yl)-1,3-dioxoisoindolin-4-yl)azetidine-3- |
| | carboxamido)ethoxy)ethoxy)acetyl)-1′,2′,3′,6′-tetrahydro-[3,4′-bipyridine]-5-carboxylate |
| TDH-087 | (R)-2-((4-fluorophenyl)amino)-2-oxo-1-phenylethyl 6-amino-1′-(2-(2-(2-(1-(2-(2,6- |
| | dioxopiperidin-3-yl)-1,3-dioxoisoindolin-5-yl)azetidine-3- |
| | carboxamido)ethoxy)ethoxy)acetyl)-1′,2′,3′,6′-tetrahydro-[3,4′-bipyridine]-5-carboxylate |
| TDH-088 | (R)-2-((4-fluorophenyl)amino)-2-oxo-1-phenylethyl 6-amino-1′-(2-(2-(2-(1-(2-(2,6- |
| | dioxopiperidin-3-yl)-1,3-dioxoisoindolin-4-yl)piperidine-4- |
| | carboxamido)ethoxy)ethoxy)acetyl)-1′,2′,3′,6′-tetrahydro-[3,4′-bipyridine]-5-carboxylate |
| TDH-089 | (R)-2-((4-fluorophenyl)amino)-2-oxo-1-phenylethyl 6-amino-1′-(2-(2-(2-(1-(2-(2,6- |
| | dioxopiperidin-3-yl)-1,3-dioxoisoindolin-5-yl)piperidine-4- |
| | carboxamido)ethoxy)ethoxy)acetyl)-1′,2′,3′,6′-tetrahydro-[3,4′-bipyridine]-5-carboxylate |
| TDH-092 | (R)-2-((4-fluorophenyl)amino)-2-oxo-1-phenylethyl 6-amino-1′-(6-(1-(2-(2,6-dioxopiperidin- |
| | 3-yl)-1,3-dioxoisoindolin-5-yl)azetidine-3-carboxamido)hexanoyl)-1′,2′,3′,6′-tetrahydro- |
| | [3,4′-bipyridine]-5-carboxylate |
| TDH-095 | (R)-2-((4-fluorophenyl)amino)-2-oxo-1-phenylethyl 3-amino-6-(1-(1-(10-((2-(2,6- |
| | dioxopiperidin-3-yl)-1,3-dioxoisoindolin-4-yl)amino)decyl)piperidin-4-yl)-1H-pyrazol-4- |
| | yl)pyrazine-2-carboxylate |
| TDH-096 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-4-yl)amino)acetamido)ethoxy)ethyl)piperidin-4-yl)- |
| | 1H-pyrazol-4-yl)pyrazine-2-carboxylate |
| TDH-101 | (R)-2-((4-fluorophenyl)amino)-2-oxo-1-phenylethyl 3-amino-6-(1-(1-(6-(2-((2-(2,6- |
| | dioxopiperidin-3-yl)-1,3-dioxoisoindolin-4-yl)amino)acetamido)hexyl)piperidin-4-yl)-1H- |
| | pyrazol-4-yl)pyrazine-2-carboxylate |
| TDH-102 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-4- |
| | yl)amino)acetamido)ethoxy)ethoxy)ethyl)piperidin-4-yl)-1H-pyrazol-4-yl)pyrazine-2- |
| | carboxylate |
| TDH-103 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-5- |
| | yl)amino)acetamido)ethoxy)ethoxy)ethyl)piperidin-4-yl)-1H-pyrazol-4-yl)pyrazine-2- |
| | carboxylate |
| TDH-104 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-4-yl)amino)ethoxy)ethoxy)acetyl)piperidin-4-yl)-1H- |
| | pyrazol-4-yl)pyrazine-2-carboxylate |
| TDH-105 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-5-yl)amino)ethoxy)ethoxy)acetyl)piperidin-4-yl)-1H- |
| | pyrazol-4-yl)pyrazine-2-carboxylate |
| TDH-107 | (R)-2-((4-fluorophenyl)amino)-2-oxo-1-phenylethyl 3-amino-6-(1-(2-(2-(2-(2-((2-(2,6- |
| | dioxopiperidin-3-yl)-1,3-dioxoisoindolin-5-yl)amino)acetamido)ethoxy)ethoxy)ethyl)- |
| | 1,2,3,6-tetrahydropyridin-4-yl)pyrazine-2-carboxylate |
| TDH-108 | (R)-2-((4-fluorophenyl)amino)-2-oxo-1-phenylethyl 3-amino-6-(1-(1-(6-(1-(2-(2,6- |
| | dioxopiperidin-3-yl)-1,3-dioxoisoindolin-4-yl)piperidine-4-carboxamido)hexyl)piperidin-4- |
| | yl)-1H-pyrazol-4-yl)pyrazine-2-carboxylate |
| TDH-109 | (R)-2-((4-fluorophenyl)amino)-2-oxo-1-phenylethyl 3-amino-6-(1-(1-(6-(1-(2-(2,6- |
| | dioxopiperidin-3-yl)-1,3-dioxoisoindolin-5-yl)piperidine-4-carboxamido)hexyl)piperidin-4- |
| | yl)-1H-pyrazol-4-yl)pyrazine-2-carboxylate |
| TDH-110 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-4-yl)piperidine-4- |
| | carboxamido)ethoxy)ethoxy)ethyl)piperidin-4-yl)-1H-pyrazol-4-yl)pyrazine-2-carboxylate |
| TDH-111 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-5-yl)piperidine-4- |
| | carboxamido)ethoxy)ethoxy)ethyl)piperidin-4-yl)-1H-pyrazol-4-yl)pyrazine-2-carboxylate |
| TDH-113 | (R)-2-((4-fluorophenyl)amino)-2-oxo-1-phenylethyl 3-amino-6-(1-(1-(6-(1-(2-(2,6- |
| | dioxopiperidin-3-yl)-1,3-dioxoisoindolin-4-yl)azetidine-3-carboxamido)hexyl)piperidin-4-yl)- |
| | 1H-pyrazol-4-yl)pyrazine-2-carboxylate |
| TDH-114 | (R)-2-((4-fluorophenyl)amino)-2-oxo-1-phenylethyl 3-amino-6-(1-(1-(6-(1-(2-(2,6- |
| | dioxopiperidin-3-yl)-1,3-dioxoisoindolin-5-yl)azetidine-3-carboxamido)hexyl)piperidin-4-yl)- |
| | 1H-pyrazol-4-yl)pyrazine-2-carboxylate |
| TDH-115 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-4-yl)azetidine-3- |
| | carboxamido)ethoxy)ethoxy)ethyl)piperidin-4-yl)-1H-pyrazol-4-yl)pyrazine-2-carboxylate |
| TDH-116 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-5-yl)azetidine-3- |
| | carboxamido)ethoxy)ethoxy)ethyl)piperidin-4-yl)-1H-pyrazol-4-yl)pyrazine-2-carboxylate |
| TDH-119 | (R)-2-((4-fluorophenyl)amino)-2-oxo-1-phenylethyl 3-amino-6-(1-(1-(6-((2-(2,6- |
| | dioxopiperidin-3-yl)-1,3-dioxoisoindolin-4-yl)amino)hexyl)piperidin-4-yl)-1H-pyrazol-4- |
| | yl)pyrazine-2-carboxylate |
| TDH-120 | (R)-2-((4-fluorophenyl)amino)-2-oxo-1-phenylethyl 3-amino-6-(1-(1-(6-((2-(2,6- |
| | dioxopiperidin-3-yl)-1,3-dioxoisoindolin-5-yl)amino)hexyl)piperidin-4-yl)-1H-pyrazol-4- |
| | yl)pyrazine-2-carboxylate |
| TDH-121 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-4-yl)amino)ethoxy)ethoxy)ethyl)piperidin-4-yl)-1H- |
| | pyrazol-4-yl)pyrazine-2-carboxylate |
| TDH-122 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-5-yl)amino)ethoxy)ethoxy)ethyl)piperidin-4-yl)-1H- |
| | pyrazol-4-yl)pyrazine-2-carboxylate |
| TDH-123 | (R)-2-((4-fluorophenyl)amino)-2-oxo-1-phenylethyl 3-amino-6-(1-(1-(6-(2-((2-(2,6- |
| | dioxopiperidin-3-yl)-1,3-dioxoisoindolin-5-yl)amino)acetamido)hexanoyl)piperidin-4-yl)-1H- |
| | pyrazol-4-yl)pyrazine-2-carboxylate |
| TDH-124 | (R)-2-((4-fluorophenyl)amino)-2-oxo-1-phenylethyl 3-amino-6-(1-(1-(6-(2-((2-(2,6- |
| | dioxopiperidin-3-yl)-1,3-dioxoisoindolin-4-yl)amino)acetamido)hexanoyl)piperidin-4-yl)-1H- |
| | pyrazol-4-yl)pyrazine-2-carboxylate |
| TDH-125 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-5-yl)amino)acetamido)ethoxy)ethyl)piperidin-4-yl)- |
| | 1H-pyrazol-4-yl)pyrazine-2-carboxylate |
| TDH-126 | (R)-2-((4-fluorophenyl)amino)-2-oxo-1-phenylethyl 3-amino-6-(1-(1-(2-(2-(1-(2-(2,6- |
| | dioxopiperidin-3-yl)-1,3-dioxoisoindolin-4-yl)azetidine-3- |
| | carboxamido)ethoxy)ethyl)piperidin-4-yl)-1H-pyrazol-4-yl)pyrazine-2-carboxylate |
| TDH-127 | (R)-2-((4-fluorophenyl)amino)-2-oxo-1-phenylethyl 3-amino-6-(1-(1-(2-(2-(1-(2-(2,6- |
| | dioxopiperidin-3-yl)-1,3-dioxoisoindolin-5-yl)azetidine-3- |
| | carboxamido)ethoxy)ethyl)piperidin-4-yl)-1H-pyrazol-4-yl)pyrazine-2-carboxylate |
| TDH-128 | (R)-2-((4-fluorophenyl)amino)-2-oxo-1-phenylethyl 3-amino-6-(1-(1-(2-(2-(1-(2-(2,6- |
| | dioxopiperidin-3-yl)-1,3-dioxoisoindolin-4-yl)piperidine-4- |
| | carboxamido)ethoxy)ethyl)piperidin-4-yl)-1H-pyrazol-4-yl)pyrazine-2-carboxylate |
| TDH-129 | (R)-2-((4-fluorophenyl)amino)-2-oxo-1-phenylethyl 3-amino-6-(1-(1-(2-(2-(1-(2-(2,6- |
| | dioxopiperidin-3-yl)-1,3-dioxoisoindolin-5-yl)piperidine-4- |
| | carboxamido)ethoxy)ethyl)piperidin-4-yl)-1H-pyrazol-4-yl)pyrazine-2-carboxylate |
| TDH-130 | (R)-2-((4-fluorophenyl)amino)-2-oxo-1-phenylethyl 3-amino-6-(1-(1-(2-(2-((2-(2,6- |
| | dioxopiperidin-3-yl)-1,3-dioxoisoindolin-4-yl)amino)ethoxy)ethyl)piperidin-4-yl)-1H-pyrazol- |
| | 4-yl)pyrazine-2-carboxylate |
| TDH-131 | (R)-2-((4-fluorophenyl)amino)-2-oxo-1-phenylethyl 3-amino-6-(1-(1-(2-(2-((2-(2,6- |
| | dioxopiperidin-3-yl)-1,3-dioxoisoindolin-5-yl)amino)ethoxy)ethyl)piperidin-4-yl)-1H-pyrazol- |
| | 4-yl)pyrazine-2-carboxylate |
| TDH-132 | (R)-2-((4-fluorophenyl)amino)-2-oxo-1-phenylethyl 3-amino-6-(1-(1-(6-(2-((2-(2,6- |
| | dioxopiperidin-3-yl)-1,3-dioxoisoindolin-5-yl)amino)acetamido)hexyl)piperidin-4-yl)-1H- |
| | pyrazol-4-yl)pyrazine-2-carboxylate |
| TDH-133 | (R)-2-((4-fluorophenyl)amino)-2-oxo-1-phenylethyl 3-amino-6-(1-(1-(6-(2-((2-(2,6- |
| | dioxopiperidin-3-yl)-1,3-dioxoisoindolin-4-yl)oxy)acetamido)hexyl)piperidin-4-yl)-1H- |
| | pyrazol-4-yl)pyrazine-2-carboxylate |
| TDH-134 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-4-yl)oxy)acetamido)ethoxy)ethyl)piperidin-4-yl)-1H- |
| | pyrazol-4-yl)pyrazine-2-carboxylate |
| TDH-135 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-4-yl)oxy)acetamido)ethoxy)ethoxy)ethyl)piperidin- |
| | 4-yl)-1H-pyrazol-4-yl)pyrazine-2-carboxylate |
| TDH-136 | (R)-2-((4-fluorophenyl)amino)-2-oxo-1-phenylethyl 3-amino-6-(1-(1-((1-((2-(2,6- |
| | dioxopiperidin-3-yl)-1,3-dioxoisoindolin-4-yl)glycyl)piperidin-4-yl)methyl)piperidin-4-yl)-1H- |
| | pyrazol-4-yl)pyrazine-2-carboxylate |
| TDH-137 | (R)-2-((4-fluorophenyl)amino)-2-oxo-1-phenylethyl 3-amino-6-(1-(1-((1-((2-(2,6- |
| | dioxopiperidin-3-yl)-1,3-dioxoisoindolin-5-yl)glycyl)piperidin-4-yl)methyl)piperidin-4-yl)-1H- |
| | pyrazol-4-yl)pyrazine-2-carboxylate |
| TDH-138 | (R)-2-((4-fluorophenyl)amino)-2-oxo-1-phenylethyl 3-amino-6-(1-(1-((1-(1-(2-(2,6- |
| | dioxopiperidin-3-yl)-1,3-dioxoisoindolin-4-yl)azetidine-3-carbonyl)piperidin-4- |
| | yl)methyl)piperidin-4-yl)-1H-pyrazol-4-yl)pyrazine-2-carboxylate |
| TDH-139 | (R)-2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-4-yl)piperidin-4-yl)-1-oxo-5,8,11-trioxa-2- |
| | azatridecan-13-yl)piperidin-4-yl)-1H-pyrazol-4-yl)pyrazine-2-carboxylate |
| TDH-140 | (R)-2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-5-yl)piperidin-4-yl)-1-oxo-5,8,11-trioxa-2- |
| | azatridecan-13-yl)piperidin-4-yl)-1H-pyrazol-4-yl)pyrazine-2-carboxylate |
| TDH-141 | (R)-2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-4-yl)azetidin-3-yl)-1-oxo-5,8,11-trioxa-2- |
| | azatridecan-13-yl)piperidin-4-yl)-1H-pyrazol-4-yl)pyrazine-2-carboxylate |
| TDH-142 | (R)-2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-5-yl)azetidin-3-yl)-1-oxo-5,8,11-trioxa-2- |
| | azatridecan-13-yl)piperidin-4-yl)-1H-pyrazol-4-yl)pyrazine-2-carboxylate |
| TDH-143 | (R)-2-((4-fluorophenyl)amino)-2-oxo-1-phenylethyl 3-amino-6-(1-(1-(1-((2-(2,6- |
| | dioxopiperidin-3-yl)-1,3-dioxoisoindolin-4-yl)amino)-2-oxo-6,9,12-trioxa-3-azatetradecan- |
| | 14-yl)piperidin-4-yl)-1H-pyrazol-4-yl)pyrazine-2-carboxylate |
| TDH-144 | (R)-2-((4-fluorophenyl)amino)-2-oxo-1-phenylethyl 3-amino-6-(1-(1-(1-((2-(2,6- |
| | dioxopiperidin-3-yl)-1,3-dioxoisoindolin-5-yl)amino)-2-oxo-6,9,12-trioxa-3-azatetradecan- |
| | 14-yl)piperidin-4-yl)-1H-pyrazol-4-yl)pyrazine-2-carboxylate |
| TDH-145 | (R)-2-((4-fluorophenyl)amino)-2-oxo-1-phenylethyl 3-amino-6-(1-(1-((1-(1-(2-(2,6- |
| | dioxopiperidin-3-yl)-1,3-dioxoisoindolin-5-yl)azetidine-3-carbonyl)piperidin-4- |
| | yl)methyl)piperidin-4-yl)-1H-pyrazol-4-yl)pyrazine-2-carboxylate |
| TDH-146 | (R)-2-((4-fluorophenyl)amino)-2-oxo-1-phenylethyl 3-amino-6-(1-(1-((1-(1-(2-(2,6- |
| | dioxopiperidin-3-yl)-1,3-dioxoisoindolin-4-yl)piperidine-4-carbonyl)piperidin-4- |
| | yl)methyl)piperidin-4-yl)-1H-pyrazol-4-yl)pyrazine-2-carboxylate |
| TDH-147 | (R)-2-((4-fluorophenyl)amino)-2-oxo-1-phenylethyl 3-amino-6-(1-(1-((1-(1-(2-(2,6- |
| | dioxopiperidin-3-yl)-1,3-dioxoisoindolin-5-yl)piperidine-4-carbonyl)piperidin-4- |
| | yl)methyl)piperidin-4-yl)-1H-pyrazol-4-yl)pyrazine-2-carboxylate |
| TDH-148 | (R)-2-((4-fluorophenyl)amino)-2-oxo-1-phenylethyl 3-amino-6-(1-(1-((1-(2-((2-(2,6- |
| | dioxopiperidin-3-yl)-1,3-dioxoisoindolin-4-yl)oxy)acetyl)piperidin-4-yl)methyl)piperidin-4-yl)- |
| | 1H-pyrazol-4-yl)pyrazine-2-carboxylate |
| TDH-149 | (R)-2-((4-fluorophenyl)amino)-2-oxo-1-phenylethyl 3-amino-6-(1-(1-((1-(2-(2,6- |
| | dioxopiperidin-3-yl)-1,3-dioxoisoindolin-4-yl)piperidin-4-yl)methyl)piperidin-4-yl)-1H- |
| | pyrazol-4-yl)pyrazine-2-carboxylate |
| TDH-150 | (R)-2-((4-fluorophenyl)amino)-2-oxo-1-phenylethyl 3-amino-6-(1-(1-((1-(2-(2,6- |
| | dioxopiperidin-3-yl)-1,3-dioxoisoindolin-5-yl)piperidin-4-yl)methyl)piperidin-4-yl)-1H- |
| | pyrazol-4-yl)pyrazine-2-carboxylate |
| TDH-151 | (R)-2-((4-fluorophenyl)amino)-2-oxo-1-phenylethyl 3-amino-6-(1-(1-((1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)methyl)piperidin-4-yl)-1H-pyrazol-4-yl)pyrazine-2-carboxylate |
| TDH-152 | (R)-2-((4-fluorophenyl)amino)-2-oxo-1-phenylethyl 3-amino-6-(1-(1-((1-((3-(2,6- |
| | dioxopiperidin-3-yl)-4-oxo-3,4-dihydrobenzo[d][1,2,3]triazin-6-yl)glycyl)piperidin-4- |
| | yl)methyl)piperidin-4-yl)-1H-pyrazol-4-yl)pyrazine-2-carboxylate |
| TDH-153 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-4-yl)amino)ethoxy)ethoxy)ethoxy)ethyl)piperidin-4- |
| | yl)-1H-pyrazol-4-yl)pyrazine-2-carboxylate |
| TDH-154 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-5-yl)amino)ethoxy)ethoxy)ethoxy)ethyl)piperidin-4- |
| | yl)-1H-pyrazol-4-yl)pyrazine-2-carboxylate |
| TDH-155 | (R)-2-((4-fluorophenyl)amino)-2-oxo-1-phenylethyl 3-amino-6-(1-(1-(1-((2-(2,6- |
| | dioxopiperidin-3-yl)-1,3-dioxoisoindolin-4-yl)oxy)-2-oxo-6,9,12-trioxa-3-azatetradecan-14- |
| | yl)piperidin-4-yl)-1H-pyrazol-4-yl)pyrazine-2-carboxylate |
| TDH-156 | (R)-2-((4-fluorophenyl)amino)-2-oxo-1-phenylethyl 3-amino-6-(1-(1-(10-(1-(2-(2,6- |
| | dioxopiperidin-3-yl)-1,3-dioxoisoindolin-4-yl)piperidine-4-carboxamido)decyl)piperidin-4- |
| | yl)-1H-pyrazol-4-yl)pyrazine-2-carboxylate |
| TDH-157 | (R)-2-((4-fluorophenyl)amino)-2-oxo-1-phenylethyl 3-amino-6-(1-(1-(10-(1-(2-(2,6- |
| | dioxopiperidin-3-yl)-1,3-dioxoisoindolin-5-yl)piperidine-4-carboxamido)decyl)piperidin-4- |
| | yl)-1H-pyrazol-4-yl)pyrazine-2-carboxylate |
| TDH-158 | (R)-2-((4-fluorophenyl)amino)-2-oxo-1-phenylethyl 3-amino-6-(1-(1-(10-(1-(2-(2,6- |
| | dioxopiperidin-3-yl)-1,3-dioxoisoindolin-4-yl)azetidine-3-carboxamido)decyl)piperidin-4-yl)- |
| | 1H-pyrazol-4-yl)pyrazine-2-carboxylate |
| TDH-159 | (R)-2-((4-fluorophenyl)amino)-2-oxo-1-phenylethyl 3-amino-6-(1-(1-(10-(1-(2-(2,6- |
| | dioxopiperidin-3-yl)-1,3-dioxoisoindolin-5-yl)azetidine-3-carboxamido)decyl)piperidin-4-yl)- |
| | 1H-pyrazol-4-yl)pyrazine-2-carboxylate |
| TDH-160 | (R)-2-((4-fluorophenyl)amino)-2-oxo-1-phenylethyl 3-amino-6-(1-(1-(10-((2-(2,6- |
| | dioxopiperidin-3-yl)-1,3-dioxoisoindolin-5-yl)amino)decyl)piperidin-4-yl)-1H-pyrazol-4- |
| | yl)pyrazine-2-carboxylate |
| TDH-161 | (R)-2-((4-fluorophenyl)amino)-2-oxo-1-phenylethyl 3-amino-6-(1-(1-(10-(2-((2-(2,6- |
| | dioxopiperidin-3-yl)-1,3-dioxoisoindolin-4-yl)amino)acetamido)decyl)piperidin-4-yl)-1H- |
| | pyrazol-4-yl)pyrazine-2-carboxylate |
| TDH-162 | (R)-2-((4-fluorophenyl)amino)-2-oxo-1-phenylethyl 3-amino-6-(1-(1-(10-(2-((2-(2,6- |
| | dioxopiperidin-3-yl)-1,3-dioxoisoindolin-5-yl)amino)acetamido)decyl)piperidin-4-yl)-1H- |
| | pyrazol-4-yl)pyrazine-2-carboxylate |
| TDH-163 | (R)-2-((4-fluorophenyl)amino)-2-oxo-1-phenylethyl 3-amino-6-(1-(1-(10-(2-((2-(2,6- |
| | dioxopiperidin-3-yl)-1,3-dioxoisoindolin-4-yl)oxy)acetamido)decyl)piperidin-4-yl)-1H- |
| | pyrazol-4-yl)pyrazine-2-carboxylate |
| TDH-164 | (R)-2-((4-fluorophenyl)amino)-2-oxo-1-phenylethyl 3-amino-6-(1-(1-(10-(2-((2-(2,6- |
| | dioxopiperidin-3-yl)-1,3-dioxoisoindolin-5-yl)oxy)acetamido)decyl)piperidin-4-yl)-1H- |
| | pyrazol-4-yl)pyrazine-2-carboxylate |
| TDH-165 | (R)-2-((4-fluorophenyl)amino)-2-oxo-1-phenylethyl 3-amino-6-(1-(1-(6-(2-((2-(2,6- |
| | dioxopiperidin-3-yl)-1,3-dioxoisoindolin-5-yl)oxy)acetamido)hexyl)piperidin-4-yl)-1H- |
| | pyrazol-4-yl)pyrazine-2-carboxylate |
| TDH-166 | (R)-2-((4-fluorophenyl)amino)-2-oxo-1-phenylethyl 3-amino-6-(1-(1-(1-((2-(2,6- |
| | dioxopiperidin-3-yl)-1,3-dioxoisoindolin-5-yl)oxy)-2-oxo-6,9,12-trioxa-3-azatetradecan-14- |
| | yl)piperidin-4-yl)-1H-pyrazol-4-yl)pyrazine-2-carboxylate |
| TDH-167 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-5-yl)oxy)acetamido)ethoxy)ethoxy)ethyl)piperidin- |
| | 4-yl)-1H-pyrazol-4-yl)pyrazine-2-carboxylate |
| TDH-168 | (R)-2-((4-fluorophenyl)amino)-2-oxo-1-phenylethyl 3-amino-6-(1-(1-(2-(2-(2-((2-(2,6- |
| | dioxopiperidin-3-yl)-1,3-dioxoisoindolin-5-yl)oxy)acetamido)ethoxy)ethyl)piperidin-4-yl)-1H- |
| | pyrazol-4-yl)pyrazine-2-carboxylate |
| TDH-169 | (R)-2-((4-fluorophenyl)amino)-2-oxo-1-phenylethyl 3-amino-6-(1-(1-((1-(2-((3-(2,6- |
| | dioxopiperidin-3-yl)-4-oxo-3,4-dihydrobenzo[d][1,2,3]triazin-6-yl)oxy)acetyl)piperidin-4- |
| | yl)methyl)piperidin-4-yl)-1H-pyrazol-4-yl)pyrazine-2-carboxylate |
| TDH-170 | (R)-2-((4-fluorophenyl)amino)-2-oxo-1-phenylethyl 3-amino-6-(1-(1-((1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)methyl)piperidin-4-yl)-1H-pyrazol-4-yl)pyrazine-2-carboxylate |
| TDH-171 | (R)-2-((4-fluorophenyl)amino)-2-oxo-1-phenylethyl 3-amino-6-(1-(1-((1-(2-((2-(2,6- |
| | dioxopiperidin-3-yl)-1,3-dioxoisoindolin-5-yl)oxy)acetyl)piperidin-4-yl)methyl)piperidin-4-yl)- |
| | 1H-pyrazol-4-yl)pyrazine-2-carboxylate |
| TDH-172 | (R)-2-((4-fluorophenyl)amino)-2-oxo-1-phenylethyl 3-amino-6-(1-(1-((1-(3-(2,6- |
| | dioxopiperidin-3-yl)-4-oxo-3,4-dihydrobenzo[d][1,2,3]triazin-6-yl)piperidin-4- |
| | yl)methyl)piperidin-4-yl)-1H-pyrazol-4-yl)pyrazine-2-carboxylate |
| TDH-173 | (R)-2-((4-fluorophenyl)amino)-2-oxo-1-phenylethyl 3-amino-6-(1-(1-(10-(2-((3-(2,6- |
| | dioxopiperidin-3-yl)-4-oxo-3,4-dihydrobenzo[d][1,2,3]triazin-6- |
| | yl)amino)acetamido)decyl)piperidin-4-yl)-1H-pyrazol-4-yl)pyrazine-2-carboxylate |
| TDH-174 | (R)-2-((4-fluorophenyl)amino)-2-oxo-1-phenylethyl 3-amino-6-(1-(1-(2-(2-(2-(2-((3-(2,6- |
| | dioxopiperidin-3-yl)-4-oxo-3,4-dihydrobenzo[d][1,2,3]triazin-6- |
| | yl)amino)acetamido)ethoxy)ethoxy)ethyl)piperidin-4-yl)-1H-pyrazol-4-yl)pyrazine-2- |
| | carboxylate |
| TDH-175 | (R)-2-((4-fluorophenyl)amino)-2-oxo-1-phenylethyl 3-amino-6-(1-(1-(1-((3-(2,6- |
| | dioxopiperidin-3-yl)-4-oxo-3,4-dihydrobenzo[d][1,2,3]triazin-6-yl)amino)-2-oxo-6,9,12- |
| | trioxa-3-azatetradecan-14-yl)piperidin-4-yl)-1H-pyrazol-4-yl)pyrazine-2-carboxylate |
| TDH-176 | (R)-2-((4-fluorophenyl)amino)-2-oxo-1-phenylethyl 3-amino-6-(1-(1-(10-(2-((3-(2,6- |
| | dioxopiperidin-3-yl)-4-oxo-3,4-dihydrobenzo[d][1,2,3]triazin-6- |
| | yl)oxy)acetamido)decyl)piperidin-4-yl)-1H-pyrazol-4-yl)pyrazine-2-carboxylate |
| TDH-177 | (R)-2-((4-fluorophenyl)amino)-2-oxo-1-phenylethyl 3-amino-6-(1-(1-(10-(1-(3-(2,6- |
| | dioxopiperidin-3-yl)-4-oxo-3,4-dihydrobenzo[d][1,2,3]triazin-6-yl)piperidine-4- |
| | carboxamido)decyl)piperidin-4-yl)-1H-pyrazol-4-yl)pyrazine-2-carboxylate |
| TDH-178 | (R)-2-((4-fluorophenyl)amino)-2-oxo-1-phenylethyl 3-amino-6-(1-(1-(10-(1-(3-(2,6- |
| | dioxopiperidin-3-yl)-4-oxo-3,4-dihydrobenzo[d][1,2,3]triazin-6-yl)azetidine-3- |
| | carboxamido)decyl)piperidin-4-yl)-1H-pyrazol-4-yl)pyrazine-2-carboxylate |
| TDH-179 | (R)-2-((4-fluorophenyl)amino)-2-oxo-1-phenylethyl 3-amino-6-(1-(1-(10-((3-(2,6- |
| | dioxopiperidin-3-yl)-4-oxo-3,4-dihydrobenzo[d][1,2,3]triazin-6-yl)amino)decyl)piperidin-4- |
| | yl)-1H-pyrazol-4-yl)pyrazine-2-carboxylate |
| TDH-186 | 3-amino-6-(1-(1-((1-(2-((2-(2,6-dioxopiperidin-3-yl)-1,3-dioxoisoindolin-4- |
| | yl)oxy)acetyl)piperidin-4-yl)methyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-187 | 3-amino-6-(1-(1-((1-(2-((2-(2,6-dioxopiperidin-3-yl)-1,3-dioxoisoindolin-5- |
| | yl)oxy)acetyl)piperidin-4-yl)methyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-188 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-4-yl)oxy)acetamido)ethoxy)ethoxy)acetyl)piperidin- |
| | 4-yl)-1H-pyrazol-4-yl)pyrazine-2-carboxylate |
| TDH-189 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-5-yl)oxy)acetamido)ethoxy)ethoxy)acetyl)piperidin- |
| | 4-yl)-1H-pyrazol-4-yl)pyrazine-2-carboxylate |
| TDH-190 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-4- |
| | yl)amino)acetamido)ethoxy)ethoxy)acetyl)piperidin-4-yl)-1H-pyrazol-4-yl)pyrazine-2- |
| | carboxylate |
| TDH-191 | (R)-2-((4-fluorophenyl)amino)-2-oxo-1-phenylethyl 3-amino-6-(1-(1-(2-(2-(2-(2-((2-(2,6- |
| | dioxopiperidin-3-yl)-1,3-dioxoisoindolin-5- |
| | yl)amino)acetamido)ethoxy)ethoxy)acetyl)piperidin-4-yl)-1H-pyrazol-4-yl)pyrazine-2- |
| | carboxylate |
| TDH-192 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-4-yl)azetidine-3- |
| | carboxamido)ethoxy)ethoxy)acetyl)piperidin-4-yl)-1H-pyrazol-4-yl)pyrazine-2-carboxylate |
| TDH-193 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-5-yl)azetidine-3- |
| | carboxamido)ethoxy)ethoxy)acetyl)piperidin-4-yl)-1H-pyrazol-4-yl)pyrazine-2-carboxylate |
| TDH-194 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-4-yl)piperidine-4- |
| | carboxamido)ethoxy)ethoxy)acetyl)piperidin-4-yl)-1H-pyrazol-4-yl)pyrazine-2-carboxylate |
| TDH-195 | (R)-2-((4-fluorophenyl)amino)-2-oxo-1-phenylethyl 3-amino-6-(1-(1-(2-(2-(2-(1-(2-(2,6- |
| | dioxopiperidin-3-yl)-1,3-dioxoisoindolin-5-yl)piperidine-4- |
| | carboxamido)ethoxy)ethoxy)acetyl)piperidin-4-yl)-1H-pyrazol-4-yl)pyrazine-2-carboxylate |
| TDH-196 | (R)-2-((4-fluorophenyl)amino)-2-oxo-1-phenylethyl 3-amino-6-(1-(1-(2-(1-(1-(2-(2,6- |
| | dioxopiperidin-3-yl)-1,3-dioxoisoindolin-4-yl)piperidine-4-carbonyl)piperidin-4- |
| | yl)ethyl)piperidin-4-yl)-1H-pyrazol-4-yl)pyrazine-2-carboxylate |
| TDH-197 | (R)-2-((4-fluorophenyl)amino)-2-oxo-1-phenylethyl 3-amino-6-(1-(1-(2-(1-(1-(2-(2,6- |
| | dioxopiperidin-3-yl)-1,3-dioxoisoindolin-5-yl)piperidine-4-carbonyl)piperidin-4- |
| | yl)ethyl)piperidin-4-yl)-1H-pyrazol-4-yl)pyrazine-2-carboxylate |
| TDH-198 | (R)-2-((4-fluorophenyl)amino)-2-oxo-1-phenylethyl 3-amino-6-(1-(1-(2-(1-((2-(2,6- |
| | dioxopiperidin-3-yl)-1,3-dioxoisoindolin-4-yl)glycyl)piperidin-4-yl)ethyl)piperidin-4-yl)-1H- |
| | pyrazol-4-yl)pyrazine-2-carboxylate |
| TDH-199 | (R)-2-((4-fluorophenyl)amino)-2-oxo-1-phenylethyl 3-amino-6-(1-(1-(2-(1-((2-(2,6- |
| | dioxopiperidin-3-yl)-1,3-dioxoisoindolin-5-yl)glycyl)piperidin-4-yl)ethyl)piperidin-4-yl)-1H- |
| | pyrazol-4-yl)pyrazine-2-carboxylate |
| TDH-200 | (R)-2-((4-fluorophenyl)amino)-2-oxo-1-phenylethyl 3-amino-6-(1-(1-(2-(1-(2-((2-(2,6- |
| | dioxopiperidin-3-yl)-1,3-dioxoisoindolin-4-yl)oxy)acetyl)piperidin-4-yl)ethyl)piperidin-4-yl)- |
| | 1H-pyrazol-4-yl)pyrazine-2-carboxylate |
| TDH-201 | (R)-2-((4-fluorophenyl)amino)-2-oxo-1-phenylethyl 3-amino-6-(1-(1-(2-(1-(2-((2-(2,6- |
| | dioxopiperidin-3-yl)-1,3-dioxoisoindolin-5-yl)oxy)acetyl)piperidin-4-yl)ethyl)piperidin-4-yl)- |
| | 1H-pyrazol-4-yl)pyrazine-2-carboxylate |
| TDH-202 | (R)-2-((4-fluorophenyl)amino)-2-oxo-1-phenylethyl 3-amino-6-(1-(1-(2-(1-(2-(2,6- |
| | dioxopiperidin-3-yl)-1,3-dioxoisoindolin-4-yl)piperidin-4-yl)ethyl)piperidin-4-yl)-1H-pyrazol- |
| | 4-yl)pyrazine-2-carboxylate |
| TDH-203 | (R)-2-((4-fluorophenyl)amino)-2-oxo-1-phenylethyl 3-amino-6-(1-(1-(2-(1-(2-(2,6- |
| | dioxopiperidin-3-yl)-1,3-dioxoisoindolin-5-yl)piperidin-4-yl)ethyl)piperidin-4-yl)-1H-pyrazol- |
| | 4-yl)pyrazine-2-carboxylate |
| TDH-204 | (R)-2-((4-fluorophenyl)amino)-2-oxo-1-phenylethyl 3-amino-6-(1-(1-((1-(2-(1-(2-(2,6- |
| | dioxopiperidin-3-yl)-1,3-dioxoisoindolin-4-yl)piperidin-4-yl)acetyl)piperidin-4- |
| | yl)methyl)piperidin-4-yl)-1H-pyrazol-4-yl)pyrazine-2-carboxylate |
| TDH-205 | (R)-2-((4-fluorophenyl)amino)-2-oxo-1-phenylethyl 3-amino-6-(1-(1-((1-(2-(1-(2-(2,6- |
| | dioxopiperidin-3-yl)-1,3-dioxoisoindolin-5-yl)piperidin-4-yl)acetyl)piperidin-4- |
| | yl)methyl)piperidin-4-yl)-1H-pyrazol-4-yl)pyrazine-2-carboxylate |
| TDH-206 | 3-amino-6-(1-(1-((1-((2-(2,6-dioxopiperidin-3-yl)-1,3-dioxoisoindolin-4-yl)glycyl)piperidin-4- |
| | yl)methyl)piperidin-4-yl)-1H-pyrazol-4-yl)-N-((R)-2-((4-fluorophenyl)amino)-2-oxo-1- |
| | phenylethyl)pyrazine-2-carboxamide |
| TDH-207 | 3-amino-6-(1-(1-((1-((2-(2,6-dioxopiperidin-3-yl)-1,3-dioxoisoindolin-5-yl)glycyl)piperidin-4- |
| | yl)methyl)piperidin-4-yl)-1H-pyrazol-4-yl)-N-((R)-2-((4-fluorophenyl)amino)-2-oxo-1- |
| | phenylethyl)pyrazine-2-carboxamide |
| TDH-208 | 3-amino-6-(1-(1-((1-(2-(2,6-dioxopiperidin-3-yl)-1,3-dioxoisoindolin-4-yl)piperidin-4- |
| | yl)methyl)piperidin-4-yl)-1H-pyrazol-4-yl)-N-((R)-2-((4-fluorophenyl)amino)-2-oxo-1- |
| | phenylethyl)pyrazine-2-carboxamide |
| TDH-209 | 3-amino-6-(1-(1-((1-(1-(2-(2,6-dioxopiperidin-3-yl)-1,3-dioxoisoindolin-4-yl)piperidine-4- |
| | carbonyl)piperidin-4-yl)methyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-210 | 3-amino-6-(1-(1-((1-(1-(2-(2,6-dioxopiperidin-3-yl)-1,3-dioxoisoindolin-5-yl)piperidine-4- |
| | carbonyl)piperidin-4-yl)methyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-211 | (R)-2-((4-fluorophenyl)amino)-2-oxo-1-phenylethyl 3-amino-6-(1-(1-((1-(2-(((S)-1-((2S,4R)- |
| | 4-hydroxy-2-((4-(4-methylthiazol-5-yl)benzyl)carbamoyl)pyrrolidin-1-yl)-3,3-dimethyl-1- |
| | oxobutan-2-yl)amino)-2-oxoethyl)piperidin-4-yl)methyl)piperidin-4-yl)-1H-pyrazol-4- |
| | yl)pyrazine-2-carboxylate |
| TDH-212 | (R)-2-((4-fluorophenyl)amino)-2-oxo-1-phenylethyl 3-amino-6-(1-(1-((1-(4-(((S)-1-((2S,4R)- |
| | 4-hydroxy-2-((4-(4-methylthiazol-5-yl)benzyl)carbamoyl)pyrrolidin-1-yl)-3,3-dimethyl-1- |
| | oxobutan-2-yl)amino)-4-oxobutanoyl)piperidin-4-yl)methyl)piperidin-4-yl)-1H-pyrazol-4- |
| | yl)pyrazine-2-carboxylate |
| TDH-213 | 3-amino-6-(1-(1-(2-(2-(2-(1-(2-(2,6-dioxopiperidin-3-yl)-1,3-dioxoisoindolin-4-yl)piperidine- |
| | 4-carboxamido)ethoxy)ethoxy)ethyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-214 | 3-amino-6-(1-(1-(2-(2-(2-(1-(2-(2,6-dioxopiperidin-3-yl)-1,3-dioxoisoindolin-5-yl)piperidine- |
| | 4-carboxamido)ethoxy)ethoxy)ethyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-215 | 3-amino-6-(1-(1-(2-(2-(2-((2-(2,6-dioxopiperidin-3-yl)-1,3-dioxoisoindolin-4- |
| | yl)amino)ethoxy)ethoxy)ethyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-216 | 3-amino-6-(1-(1-(2-(2-(2-((2-(2,6-dioxopiperidin-3-yl)-1,3-dioxoisoindolin-5- |
| | yl)amino)ethoxy)ethoxy)ethyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-217 | 3-amino-6-(1-(1-(2-(2-(2-(2-((2-(2,6-dioxopiperidin-3-yl)-1,3-dioxoisoindolin-4- |
| | yl)amino)acetamido)ethoxy)ethoxy)ethyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-218 | 3-amino-6-(1-(1-(2-(2-(2-(2-((2-(2,6-dioxopiperidin-3-yl)-1,3-dioxoisoindolin-5- |
| | yl)amino)acetamido)ethoxy)ethoxy)ethyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-221 | 2-((4-fluorophenyl)amino)-2-oxo-1-phenylethyl 3-amino-6-(1-(1-((1-(1-(2-(2,6- |
| | dioxopiperidin-3-yl)-1,3-dioxoisoindolin-5-yl)piperidine-4-carbonyl)piperidin-4- |
| | yl)methyl)piperidin-4-yl)-1H-1,2,3-triazol-4-yl)pyrazine-2-carboxylate |
| TDH-222 | 2-((4-fluorophenyl)amino)-2-oxo-1-phenylethyl 3-amino-6-(1-(1-((1-(1-(2-(2,6- |
| | dioxopiperidin-3-yl)-1,3-dioxoisoindolin-4-yl)piperidine-4-carbonyl)piperidin-4- |
| | yl)methyl)piperidin-4-yl)-1H-1,2,3-triazol-4-yl)pyrazine-2-carboxylate |
| TDH-223 | 2-((4-fluorophenyl)amino)-2-oxo-1-phenylethyl 3-amino-6-(1-(1-((1-((2-(2,6-dioxopiperidin- |
| | 3-yl)-1,3-dioxoisoindolin-5-yl)glycyl)piperidin-4-yl)methyl)piperidin-4-yl)-1H-1,2,3-triazol-4- |
| | yl)pyrazine-2-carboxylate |
| TDH-224 | 2-((4-fluorophenyl)amino)-2-oxo-1-phenylethyl 3-amino-6-(1-(1-((1-((2-(2,6-dioxopiperidin- |
| | 3-yl)-1,3-dioxoisoindolin-4-yl)glycyl)piperidin-4-yl)methyl)piperidin-4-yl)-1H-1,2,3-triazol-4- |
| | yl)pyrazine-2-carboxylate |
| TDH-225 | 2-((4-fluorophenyl)amino)-2-oxo-1-phenylethyl 3-amino-6-(1-(1-((1-(2-(2,6-dioxopiperidin- |
| | 3-yl)-1,3-dioxoisoindolin-4-yl)piperidin-4-yl)methyl)piperidin-4-yl)-1H-1,2,3-triazol-4- |
| | yl)pyrazine-2-carboxylate |
| TDH-226 | 2-((4-fluorophenyl)amino)-2-oxo-1-phenylethyl 3-amino-6-(1-(1-((1-(2-(2,6-dioxopiperidin- |
| | 3-yl)-1,3-dioxoisoindolin-5-yl)piperidin-4-yl)methyl)piperidin-4-yl)-1H-1,2,3-triazol-4- |
| | yl)pyrazine-2-carboxylate |
| TDH-230 | 2-((4-fluorophenyl)amino)-2-oxo-1-phenylethyl 3-amino-6-(6-(4-((1-(1-(2-(2,6- |
| | dioxopiperidin-3-yl)-1,3-dioxoisoindolin-4-yl)piperidine-4-carbonyl)piperidin-4- |
| | yl)methyl)piperazin-1-yl)pyridin-3-yl)pyrazine-2-carboxylate |
| TDH-231 | 2-((4-fluorophenyl)amino)-2-oxo-1-phenylethyl 3-amino-6-(6-(4-((1-(1-(2-(2,6- |
| | dioxopiperidin-3-yl)-1,3-dioxoisoindolin-5-yl)piperidine-4-carbonyl)piperidin-4- |
| | yl)methyl)piperazin-1-yl)pyridin-3-yl)pyrazine-2-carboxylate |
| TDH-232 | 2-((4-fluorophenyl)amino)-2-oxo-1-phenylethyl 3-amino-6-(6-(4-((1-((2-(2,6-dioxopiperidin- |
| | 3-yl)-1,3-dioxoisoindolin-4-yl)glycyl)piperidin-4-yl)methyl)piperazin-1-yl)pyridin-3- |
| | yl)pyrazine-2-carboxylate |
| TDH-233 | 2-((4-fluorophenyl)amino)-2-oxo-1-phenylethyl 3-amino-6-(6-(4-((1-((2-(2,6-dioxopiperidin- |
| | 3-yl)-1,3-dioxoisoindolin-5-yl)glycyl)piperidin-4-yl)methyl)piperazin-1-yl)pyridin-3- |
| | yl)pyrazine-2-carboxylate |
| TDH-234 | 2-((4-fluorophenyl)amino)-2-oxo-1-phenylethyl 3-amino-6-(1-(1-((1-(2-((2-((2-(2,6- |
| | dioxopiperidin-3-yl)-1,3-dioxoisoindolin-4-yl)amino)ethyl)amino)-2-oxoethyl)piperidin-4- |
| | yl)methyl)piperidin-4-yl)-1H-pyrazol-4-yl)pyrazine-2-carboxylate |
| TDH-235 | 2-((4-fluorophenyl)amino)-2-oxo-1-phenylethyl 3-amino-6-(1-(1-((1-(2-((3-((2-(2,6- |
| | dioxopiperidin-3-yl)-1,3-dioxoisoindolin-4-yl)amino)propyl)amino)-2-oxoethyl)piperidin-4- |
| | yl)methyl)piperidin-4-yl)-1H-pyrazol-4-yl)pyrazine-2-carboxylate |
| TDH-236 | 2-((4-fluorophenyl)amino)-2-oxo-1-phenylethyl 3-amino-6-(1-(1-((1-(2-((1-(2-(2,6- |
| | dioxopiperidin-3-yl)-1,3-dioxoisoindolin-4-yl)piperidin-4-yl)amino)-2-oxoethyl)piperidin-4- |
| | yl)methyl)piperidin-4-yl)-1H-pyrazol-4-yl)pyrazine-2-carboxylate |
| TDH-237 | 2-((4-fluorophenyl)amino)-2-oxo-1-phenylethyl 3-amino-6-(1-(1-((1-(2-(4-(2-(2,6- |
| | dioxopiperidin-3-yl)-1,3-dioxoisoindolin-4-yl)piperazin-1-yl)-2-oxoethyl)piperidin-4- |
| | yl)methyl)piperidin-4-yl)-1H-pyrazol-4-yl)pyrazine-2-carboxylate |
| TDH-238 | 2-((4-fluorophenyl)amino)-2-oxo-1-phenylethyl 3-amino-6-(1-(1-((1-(2-(4-((2-(2,6- |
| | dioxopiperidin-3-yl)-1,3-dioxoisoindolin-4-yl)amino)piperidin-1-yl)-2-oxoethyl)piperidin-4- |
| | yl)methyl)piperidin-4-yl)-1H-pyrazol-4-yl)pyrazine-2-carboxylate |
| TDH-239 | 2-((4-fluorophenyl)amino)-2-oxo-1-phenylethyl 3-amino-6-(1-(1-(3-(4-(1-(2-(2,6- |
| | dioxopiperidin-3-yl)-1,3-dioxoisoindolin-4-yl)piperidine-4-carbonyl)piperazin-1- |
| | yl)propyl)piperidin-4-yl)-1H-pyrazol-4-yl)pyrazine-2-carboxylate |
| TDH-240 | 2-((4-fluorophenyl)amino)-2-oxo-1-phenylethyl 3-amino-6-(1-(1-(3-(4-(1-(2-(2,6- |
| | dioxopiperidin-3-yl)-1,3-dioxoisoindolin-5-yl)piperidine-4-carbonyl)piperazin-1- |
| | yl)propyl)piperidin-4-yl)-1H-pyrazol-4-yl)pyrazine-2-carboxylate |
| TDH-241 | 2-((4-fluorophenyl)amino)-2-oxo-1-phenylethyl 3-amino-6-(1-(1-(3-(4-((2-(2,6- |
| | dioxopiperidin-3-yl)-1,3-dioxoisoindolin-4-yl)glycyl)piperazin-1-yl)propyl)piperidin-4-yl)-1H- |
| | pyrazol-4-yl)pyrazine-2-carboxylate |
| TDH-242 | 2-((4-fluorophenyl)amino)-2-oxo-1-phenylethyl 3-amino-6-(1-(1-(3-(4-((2-(2,6- |
| | dioxopiperidin-3-yl)-1,3-dioxoisoindolin-5-yl)glycyl)piperazin-1-yl)propyl)piperidin-4-yl)-1H- |
| | pyrazol-4-yl)pyrazine-2-carboxylate |
| TDH-243 | 2-((4-fluorophenyl)amino)-2-oxo-1-phenylethyl 3-amino-6-(1-(1-(3-(4-(2-((2-(2,6- |
| | dioxopiperidin-3-yl)-1,3-dioxoisoindolin-4-yl)oxy)acetyl)piperazin-1-yl)propyl)piperidin-4- |
| | yl)-1H-pyrazol-4-yl)pyrazine-2-carboxylate |
| TDH-244 | 2-((4-fluorophenyl)amino)-2-oxo-1-phenylethyl 3-amino-6-(1-(1-(3-(4-(2-((2-(2,6- |
| | dioxopiperidin-3-yl)-1,3-dioxoisoindolin-5-yl)oxy)acetyl)piperazin-1-yl)propyl)piperidin-4- |
| | yl)-1H-pyrazol-4-yl)pyrazine-2-carboxylate |
| TDH-245 | 2-((4-fluorophenyl)amino)-2-oxo-1-phenylethyl 3-amino-6-(1-(1-(3-(4-(2-(2,6- |
| | dioxopiperidin-3-yl)-1,3-dioxoisoindolin-4-yl)piperazin-1-yl)propyl)piperidin-4-yl)-1H- |
| | pyrazol-4-yl)pyrazine-2-carboxylate |
| TDH-246 | 2-((4-fluorophenyl)amino)-2-oxo-1-phenylethyl 3-amino-6-(1-(1-(3-(4-(2-(2,6- |
| | dioxopiperidin-3-yl)-1,3-dioxoisoindolin-5-yl)piperazin-1-yl)propyl)piperidin-4-yl)-1H- |
| | pyrazol-4-yl)pyrazine-2-carboxylate |
| TDH-247 | 2-((4-fluorophenyl)amino)-2-oxo-1-phenylethyl 3-amino-6-(1-(1-(2-((1-(1-(2-(2,6- |
| | dioxopiperidin-3-yl)-1,3-dioxoisoindolin-4-yl)piperidine-4-carbonyl)piperidin-4- |
| | yl)oxy)ethyl)piperidin-4-yl)-1H-pyrazol-4-yl)pyrazine-2-carboxylate |
| TDH-248 | 2-((4-fluorophenyl)amino)-2-oxo-1-phenylethyl 3-amino-6-(1-(1-(2-((1-(1-(2-(2,6- |
| | dioxopiperidin-3-yl)-1,3-dioxoisoindolin-5-yl)piperidine-4-carbonyl)piperidin-4- |
| | yl)oxy)ethyl)piperidin-4-yl)-1H-pyrazol-4-yl)pyrazine-2-carboxylate |
| TDH-249 | 2-((4-fluorophenyl)amino)-2-oxo-1-phenylethyl 3-amino-6-(1-(1-(2-((1-((2-(2,6- |
| | dioxopiperidin-3-yl)-1,3-dioxoisoindolin-4-yl)glycyl)piperidin-4-yl)oxy)ethyl)piperidin-4-yl)- |
| | 1H-pyrazol-4-yl)pyrazine-2-carboxylate |
| TDH-250 | 2-((4-fluorophenyl)amino)-2-oxo-1-phenylethyl 3-amino-6-(1-(1-(2-((1-((2-(2,6- |
| | dioxopiperidin-3-yl)-1,3-dioxoisoindolin-5-yl)glycyl)piperidin-4-yl)oxy)ethyl)piperidin-4-yl)- |
| | 1H-pyrazol-4-yl)pyrazine-2-carboxylate |
| TDH-251 | 2-((4-fluorophenyl)amino)-2-oxo-1-phenylethyl 3-amino-6-(1-(1-(2-((1-(2-((2-(2,6- |
| | dioxopiperidin-3-yl)-1,3-dioxoisoindolin-4-yl)oxy)acetyl)piperidin-4-yl)oxy)ethyl)piperidin-4- |
| | yl)-1H-pyrazol-4-yl)pyrazine-2-carboxylate |
| TDH-252 | 2-((4-fluorophenyl)amino)-2-oxo-1-phenylethyl 3-amino-6-(1-(1-(2-((1-(2-((2-(2,6- |
| | dioxopiperidin-3-yl)-1,3-dioxoisoindolin-5-yl)oxy)acetyl)piperidin-4-yl)oxy)ethyl)piperidin-4- |
| | yl)-1H-pyrazol-4-yl)pyrazine-2-carboxylate |
| TDH-253 | 2-((4-fluorophenyl)amino)-2-oxo-1-phenylethyl 3-amino-6-(1-(1-(2-((1-(2-(2,6- |
| | dioxopiperidin-3-yl)-1,3-dioxoisoindolin-4-yl)piperidin-4-yl)oxy)ethyl)piperidin-4-yl)-1H- |
| | pyrazol-4-yl)pyrazine-2-carboxylate |
| TDH-254 | 2-((4-fluorophenyl)amino)-2-oxo-1-phenylethyl 3-amino-6-(1-(1-(2-((1-(2-(2,6- |
| | dioxopiperidin-3-yl)-1,3-dioxoisoindolin-5-yl)piperidin-4-yl)oxy)ethyl)piperidin-4-yl)-1H- |
| | pyrazol-4-yl)pyrazine-2-carboxylate |
| TDH-255 | 2-((4-fluorophenyl)amino)-2-oxo-1-phenylethyl 3-amino-6-(6-(4-((1-(2-((2-(2,6- |
| | dioxopiperidin-3-yl)-1,3-dioxoisoindolin-4-yl)oxy)acetyl)piperidin-4-yl)methyl)piperazin-1- |
| | yl)pyridin-3-yl)pyrazine-2-carboxylate |
| TDH-256 | 2-((4-fluorophenyl)amino)-2-oxo-1-phenylethyl 3-amino-6-(6-(4-((1-(2-((2-(2,6- |
| | dioxopiperidin-3-yl)-1,3-dioxoisoindolin-5-yl)oxy)acetyl)piperidin-4-yl)methyl)piperazin-1- |
| | yl)pyridin-3-yl)pyrazine-2-carboxylate |
| TDH-257 | 2-((4-fluorophenyl)amino)-2-oxo-1-phenylethyl 3-amino-6-(6-(4-(2-(2-(2-(1-(2-(2,6- |
| | dioxopiperidin-3-yl)-1,3-dioxoisoindolin-4-yl)piperidine-4- |
| | carboxamido)ethoxy)ethoxy)ethyl)piperazin-1-yl)pyridin-3-yl)pyrazine-2-carboxylate |
| TDH-258 | 2-((4-fluorophenyl)amino)-2-oxo-1-phenylethyl 3-amino-6-(6-(4-(2-(2-(2-(1-(2-(2,6- |
| | dioxopiperidin-3-yl)-1,3-dioxoisoindolin-5-yl)piperidine-4- |
| | carboxamido)ethoxy)ethoxy)ethyl)piperazin-1-yl)pyridin-3-yl)pyrazine-2-carboxylate |
| TDH-259 | 2-((4-fluorophenyl)amino)-2-oxo-1-phenylethyl 3-amino-6-(6-(4-(2-(2-(2-(2-((2-(2,6- |
| | dioxopiperidin-3-yl)-1,3-dioxoisoindolin-4- |
| | yl)amino)acetamido)ethoxy)ethoxy)ethyl)piperazin-1-yl)pyridin-3-yl)pyrazine-2-carboxylate |
| TDH-260 | 2-((4-fluorophenyl)amino)-2-oxo-1-phenylethyl 3-amino-6-(6-(4-(2-(2-(2-(2-((2-(2,6- |
| | dioxopiperidin-3-yl)-1,3-dioxoisoindolin-5- |
| | yl)amino)acetamido)ethoxy)ethoxy)ethyl)piperazin-1-yl)pyridin-3-yl)pyrazine-2-carboxylate |
| TDH-261 | 2-((4-fluorophenyl)amino)-2-oxo-1-phenylethyl 3-amino-6-(6-(4-(2-(2-(2-(2-((2-(2,6- |
| | dioxopiperidin-3-yl)-1,3-dioxoisoindolin-4-yl)oxy)acetamido)ethoxy)ethoxy)ethyl)piperazin- |
| | 1-yl)pyridin-3-yl)pyrazine-2-carboxylate |
| TDH-262 | 2-((4-fluorophenyl)amino)-2-oxo-1-phenylethyl 3-amino-6-(6-(4-(2-(2-(2-(2-((2-(2,6- |
| | dioxopiperidin-3-yl)-1,3-dioxoisoindolin-5-yl)oxy)acetamido)ethoxy)ethoxy)ethyl)piperazin- |
| | 1-yl)pyridin-3-yl)pyrazine-2-carboxylate |
| TDH-263 | 2-((4-fluorophenyl)amino)-2-oxo-1-phenylethyl 3-amino-6-(1-((3S)-1-(2-(2-(2-(2-((2-(2,6- |
| | dioxopiperidin-3-yl)-1,3-dioxoisoindolin-4- |
| | yl)amino)acetamido)ethoxy)ethoxy)ethyl)pyrrolidin-3-yl)-1H-pyrazol-4-yl)pyrazine-2- |
| | carboxylate |
| TDH-264 | 2-((4-fluorophenyl)amino)-2-oxo-1-phenylethyl 3-amino-6-(1-((3S)-1-(2-(2-(2-(2-((2-(2,6- |
| | dioxopiperidin-3-yl)-1,3-dioxoisoindolin-5- |
| | yl)amino)acetamido)ethoxy)ethoxy)ethyl)pyrrolidin-3-yl)-1H-pyrazol-4-yl)pyrazine-2- |
| | carboxylate |
| TDH-265 | 2-((4-fluorophenyl)amino)-2-oxo-1-phenylethyl 3-amino-6-(1-((3S)-1-(2-(2-(2-(2-((2-(2,6- |
| | dioxopiperidin-3-yl)-1,3-dioxoisoindolin-4-yl)oxy)acetamido)ethoxy)ethoxy)ethyl)pyrrolidin- |
| | 3-yl)-1H-pyrazol-4-yl)pyrazine-2-carboxylate |
| TDH-266 | 2-((4-fluorophenyl)amino)-2-oxo-1-phenylethyl 3-amino-6-(1-((3S)-1-(2-(2-(2-(2-((2-(2,6- |
| | dioxopiperidin-3-yl)-1,3-dioxoisoindolin-5-yl)oxy)acetamido)ethoxy)ethoxy)ethyl)pyrrolidin- |
| | 3-yl)-1H-pyrazol-4-yl)pyrazine-2-carboxylate |
| TDH-267 | 2-((4-fluorophenyl)amino)-2-oxo-1-phenylethyl 3-amino-6-(1-((3R)-1-(2-(2-(2-(2-((2-(2,6- |
| | dioxopiperidin-3-yl)-1,3-dioxoisoindolin-4- |
| | yl)amino)acetamido)ethoxy)ethoxy)ethyl)pyrrolidin-3-yl)-1H-pyrazol-4-yl)pyrazine-2- |
| | carboxylate |
| TDH-268 | 2-((4-fluorophenyl)amino)-2-oxo-1-phenylethyl 3-amino-6-(1-((3R)-1-(2-(2-(2-(2-((2-(2,6- |
| | dioxopiperidin-3-yl)-1,3-dioxoisoindolin-5- |
| | yl)amino)acetamido)ethoxy)ethoxy)ethyl)pyrrolidin-3-yl)-1H-pyrazol-4-yl)pyrazine-2- |
| | carboxylate |
| TDH-269 | 2-((4-fluorophenyl)amino)-2-oxo-1-phenylethyl 3-amino-6-(1-((3R)-1-(2-(2-(2-(2-((2-(2,6- |
| | dioxopiperidin-3-yl)-1,3-dioxoisoindolin-4-yl)oxy)acetamido)ethoxy)ethoxy)ethyl)pyrrolidin- |
| | 3-yl)-1H-pyrazol-4-yl)pyrazine-2-carboxylate |
| TDH-270 | 2-((4-fluorophenyl)amino)-2-oxo-1-phenylethyl 3-amino-6-(1-((3R)-1-(2-(2-(2-(2-((2-(2,6- |
| | dioxopiperidin-3-yl)-1,3-dioxoisoindolin-5-yl)oxy)acetamido)ethoxy)ethoxy)ethyl)pyrrolidin- |
| | 3-yl)-1H-pyrazol-4-yl)pyrazine-2-carboxylate |
| TDH-271 | 2-((4-fluorophenyl)amino)-2-oxo-1-phenylethyl 3-amino-6-(1-(1-((1-(2-(3-((2-(2,6- |
| | dioxopiperidin-3-yl)-1,3-dioxoisoindolin-4-yl)amino)azetidin-1-yl)-2-oxoethyl)piperidin-4- |
| | yl)methyl)piperidin-4-yl)-1H-pyrazol-4-yl)pyrazine-2-carboxylate |
| TDH-272 | 2-((4-fluorophenyl)amino)-2-oxo-1-phenylethyl 3-amino-6-(1-(1-((1-(2-((1-(2-(2,6- |
| | dioxopiperidin-3-yl)-1,3-dioxoisoindolin-5-yl)piperidin-4-yl)amino)-2-oxoethyl)piperidin-4- |
| | yl)methyl)piperidin-4-yl)-1H-pyrazol-4-yl)pyrazine-2-carboxylate |
| TDH-273 | 2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-4-yl)piperidine-4-carbonyl)piperidine-4- |
| | carbonyl)piperidin-4-yl)-1H-pyrazol-4-yl)pyrazine-2-carboxylate |
| TDH-274 | 2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-5-yl)piperidine-4-carbonyl)piperidine-4- |
| | carbonyl)piperidin-4-yl)-1H-pyrazol-4-yl)pyrazine-2-carboxylate |
| TDH-275 | 2-((4-fluorophenyl)amino)-2-oxo-1-phenylethyl 3-amino-6-(1-(1-(1-((2-(2,6-dioxopiperidin- |
| | 3-yl)-1,3-dioxoisoindolin-4-yl)glycyl)piperidine-4-carbonyl)piperidin-4-yl)-1H-pyrazol-4- |
| | yl)pyrazine-2-carboxylate |
| TDH-276 | 2-((4-fluorophenyl)amino)-2-oxo-1-phenylethyl 3-amino-6-(1-(1-(1-((2-(2,6-dioxopiperidin- |
| | 3-yl)-1,3-dioxoisoindolin-5-yl)glycyl)piperidine-4-carbonyl)piperidin-4-yl)-1H-pyrazol-4- |
| | yl)pyrazine-2-carboxylate |
| TDH-277 | 2-((4-fluorophenyl)amino)-2-oxo-1-phenylethyl 3-amino-6-(1-(1-(1-(2-((2-(2,6- |
| | dioxopiperidin-3-yl)-1,3-dioxoisoindolin-4-yl)oxy)acetyl)piperidine-4-carbonyl)piperidin-4- |
| | yl)-1H-pyrazol-4-yl)pyrazine-2-carboxylate |
| TDH-278 | 2-((4-fluorophenyl)amino)-2-oxo-1-phenylethyl 3-amino-6-(1-(1-(1-(2-((2-(2,6- |
| | dioxopiperidin-3-yl)-1,3-dioxoisoindolin-5-yl)oxy)acetyl)piperidine-4-carbonyl)piperidin-4- |
| | yl)-1H-pyrazol-4-yl)pyrazine-2-carboxylate |
| TDH-279 | 2-((4-fluorophenyl)amino)-2-oxo-1-phenylethyl 3-amino-6-(1-(1-(1-(2-(2,6-dioxopiperidin-3- |
| | yl)-1,3-dioxoisoindolin-4-yl)piperidine-4-carbonyl)piperidin-4-yl)-1H-pyrazol-4-yl)pyrazine- |
| | 2-carboxylate |
| TDH-280 | 2-((4-fluorophenyl)amino)-2-oxo-1-phenylethyl 3-amino-6-(1-(1-(1-(2-(2,6-dioxopiperidin-3- |
| | yl)-1,3-dioxoisoindolin-5-yl)piperidine-4-carbonyl)piperidin-4-yl)-1H-pyrazol-4-yl)pyrazine- |
| | 2-carboxylate |
| TDH-281 | 2-((4-fluorophenyl)amino)-2-oxo-1-phenylethyl 3-amino-6-(1-((3S)-1-((1-((2-(2,6- |
| | dioxopiperidin-3-yl)-1,3-dioxoisoindolin-4-yl)glycyl)piperidin-4-yl)methyl)pyrrolidin-3-yl)-1H- |
| | pyrazol-4-yl)pyrazine-2-carboxylate |
| TDH-282 | 2-((4-fluorophenyl)amino)-2-oxo-1-phenylethyl 3-amino-6-(1-((3S)-1-((1-((2-(2,6- |
| | dioxopiperidin-3-yl)-1,3-dioxoisoindolin-5-yl)glycyl)piperidin-4-yl)methyl)pyrrolidin-3-yl)-1H- |
| | pyrazol-4-yl)pyrazine-2-carboxylate |
| TDH-283 | 2-((4-fluorophenyl)amino)-2-oxo-1-phenylethyl 3-amino-6-(1-((3S)-1-((1-(2-((2-(2,6- |
| | dioxopiperidin-3-yl)-1,3-dioxoisoindolin-4-yl)oxy)acetyl)piperidin-4-yl)methyl)pyrrolidin-3- |
| | yl)-1H-pyrazol-4-yl)pyrazine-2-carboxylate |
| TDH-284 | 2-((4-fluorophenyl)amino)-2-oxo-1-phenylethyl 3-amino-6-(1-((3S)-1-((1-(2-((2-(2,6- |
| | dioxopiperidin-3-yl)-1,3-dioxoisoindolin-5-yl)oxy)acetyl)piperidin-4-yl)methyl)pyrrolidin-3- |
| | yl)-1H-pyrazol-4-yl)pyrazine-2-carboxylate |
| TDH-285 | 2-((4-fluorophenyl)amino)-2-oxo-1-phenylethyl 3-amino-6-(1-((3R)-1-((1-((2-(2,6- |
| | dioxopiperidin-3-yl)-1,3-dioxoisoindolin-4-yl)glycyl)piperidin-4-yl)methyl)pyrrolidin-3-yl)-1H- |
| | pyrazol-4-yl)pyrazine-2-carboxylate |
| TDH-286 | 2-((4-fluorophenyl)amino)-2-oxo-1-phenylethyl 3-amino-6-(1-((3R)-1-((1-((2-(2,6- |
| | dioxopiperidin-3-yl)-1,3-dioxoisoindolin-5-yl)glycyl)piperidin-4-yl)methyl)pyrrolidin-3-yl)-1H- |
| | pyrazol-4-yl)pyrazine-2-carboxylate |
| TDH-287 | 2-((4-fluorophenyl)amino)-2-oxo-1-phenylethyl 3-amino-6-(1-((3R)-1-((1-(2-((2-(2,6- |
| | dioxopiperidin-3-yl)-1,3-dioxoisoindolin-4-yl)oxy)acetyl)piperidin-4-yl)methyl)pyrrolidin-3- |
| | yl)-1H-pyrazol-4-yl)pyrazine-2-carboxylate |
| TDH-288 | 2-((4-fluorophenyl)amino)-2-oxo-1-phenylethyl 3-amino-6-(1-((3R)-1-((1-(2-((2-(2,6- |
| | dioxopiperidin-3-yl)-1,3-dioxoisoindolin-5-yl)oxy)acetyl)piperidin-4-yl)methyl)pyrrolidin-3- |
| | yl)-1H-pyrazol-4-yl)pyrazine-2-carboxylate |
| TDH-289 | 2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-5-yl)piperidine-4- |
| | carbonyl)piperidin-4-yl)ethyl)piperidin-4-yl)-1H-pyrazol-4-yl)pyrazine-2-carboxylate |
| TDH-290 | 2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)ethyl)piperidin-4-yl)-1H-pyrazol-4-yl)pyrazine-2-carboxylate |
| TDH-291 | 2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)ethyl)piperidin-4-yl)-1H-pyrazol-4-yl)pyrazine-2-carboxylate |
| TDH-292 | 2-((4-fluorophenyl)amino)-2-oxo-1-phenylethyl 3-amino-6-(1-(1-(2-(1-((3-(2,6- |
| | dioxopiperidin-3-yl)-4-oxo-3,4-dihydrobenzo[d][1,2,3]triazin-6-yl)glycyl)piperidin-4- |
| | yl)ethyl)piperidin-4-yl)-1H-pyrazol-4-yl)pyrazine-2-carboxylate |
| TDH-293 | 2-((4-fluorophenyl)amino)-2-oxo-1-phenylethyl 3-amino-6-(1-(1-(2-(1-(2-((3-(2,6- |
| | dioxopiperidin-3-yl)-4-oxo-3,4-dihydrobenzo[d][1,2,3]triazin-6-yl)oxy)acetyl)piperidin-4- |
| | yl)ethyl)piperidin-4-yl)-1H-pyrazol-4-yl)pyrazine-2-carboxylate |
| TDH-294 | 2-((4-fluorophenyl)amino)-2-oxo-1-phenylethyl 3-amino-6-(1-(1-(2-(1-(3-(2,6- |
| | dioxopiperidin-3-yl)-4-oxo-3,4-dihydrobenzo[d][1,2,3]triazin-5-yl)piperidin-4- |
| | yl)ethyl)piperidin-4-yl)-1H-pyrazol-4-yl)pyrazine-2-carboxylate |
| TDH-295 | 2-((4-fluorophenyl)amino)-2-oxo-1-phenylethyl 3-amino-6-(1-(1-(2-(1-(3-(2,6- |
| | dioxopiperidin-3-yl)-4-oxo-3,4-dihydrobenzo[d][1,2,3]triazin-6-yl)piperidin-4- |
| | yl)ethyl)piperidin-4-yl)-1H-pyrazol-4-yl)pyrazine-2-carboxylate |
| TDH-296 | 2-((4-fluorophenyl)amino)-2-oxo-1-phenylethyl 3-amino-6-(1-(1-((1-((2-(2,6-dioxopiperidin- |
| | 3-yl)-1,3-dioxoisoindolin-4-yl)glycyl)piperidin-4-yl)methyl)azetidin-3-yl)-1H-pyrazol-4- |
| | yl)pyrazine-2-carboxylate |
| TDH-297 | 2-((4-fluorophenyl)amino)-2-oxo-1-phenylethyl 3-amino-6-(1-(1-((1-((2-(2,6-dioxopiperidin- |
| | 3-yl)-1,3-dioxoisoindolin-5-yl)glycyl)piperidin-4-yl)methyl)azetidin-3-yl)-1H-pyrazol-4- |
| | yl)pyrazine-2-carboxylate |
| TDH-298 | 2-((4-fluorophenyl)amino)-2-oxo-1-phenylethyl 3-amino-6-(1-(1-((1-(2-((2-(2,6- |
| | dioxopiperidin-3-yl)-1,3-dioxoisoindolin-4-yl)oxy)acetyl)piperidin-4-yl)methyl)azetidin-3-yl)- |
| | 1H-pyrazol-4-yl)pyrazine-2-carboxylate |
| TDH-299 | 2-((4-fluorophenyl)amino)-2-oxo-1-phenylethyl 3-amino-6-(1-(1-((1-(2-((2-(2,6- |
| | dioxopiperidin-3-yl)-1,3-dioxoisoindolin-5-yl)oxy)acetyl)piperidin-4-yl)methyl)azetidin-3-yl)- |
| | 1H-pyrazol-4-yl)pyrazine-2-carboxylate |
| TDH-300 | 2-((4-fluorophenyl)amino)-2-oxo-1-phenylethyl 2-amino-5-(1-(1-(2-(1-(2-((2-(2,6- |
| | dioxopiperidin-3-yl)-1,3-dioxoisoindolin-5-yl)oxy)acetyl)piperidin-4-yl)ethyl)piperidin-4-yl)- |
| | 1H-pyrazol-4-yl)nicotinate |
| TDH-301 | 2-((4-fluorophenyl)amino)-2-oxo-1-phenylethyl 2-amino-5-(1-(1-(2-(1-(2-((2-(2,6- |
| | dioxopiperidin-3-yl)-1,3-dioxoisoindolin-4-yl)oxy)acetyl)piperidin-4-yl)ethyl)piperidin-4-yl)- |
| | 1H-pyrazol-4-yl)nicotinate |
| TDH-302 | 2-((4-fluorophenyl)amino)-2-oxo-1-phenylethyl 2-amino-5-(1-(1-(2-(1-((2-(2,6- |
| | dioxopiperidin-3-yl)-1,3-dioxoisoindolin-5-yl)glycyl)piperidin-4-yl)ethyl)piperidin-4-yl)-1H- |
| | pyrazol-4-yl)nicotinate |
| TDH-303 | 2-((4-fluorophenyl)amino)-2-oxo-1-phenylethyl 2-amino-5-(1-(1-(2-(1-((2-(2,6- |
| | dioxopiperidin-3-yl)-1,3-dioxoisoindolin-4-yl)glycyl)piperidin-4-yl)ethyl)piperidin-4-yl)-1H- |
| | pyrazol-4-yl)nicotinate |
| TDH-304 | 2-((4-fluorophenyl)amino)-2-oxo-1-phenylethyl 3-amino-6-(1-(1-((4-(1-(2-(2,6- |
| | dioxopiperidin-3-yl)-1,3-dioxoisoindolin-4-yl)piperidine-4-carbonyl)morpholin-2- |
| | yl)methyl)piperidin-4-yl)-1H-pyrazol-4-yl)pyrazine-2-carboxylate |
| TDH-305 | 2-((4-fluorophenyl)amino)-2-oxo-1-phenylethyl 3-amino-6-(1-(1-((4-(1-(2-(2,6- |
| | dioxopiperidin-3-yl)-1,3-dioxoisoindolin-5-yl)piperidine-4-carbonyl)morpholin-2- |
| | yl)methyl)piperidin-4-yl)-1H-pyrazol-4-yl)pyrazine-2-carboxylate |
| TDH-306 | 2-((4-fluorophenyl)amino)-2-oxo-1-phenylethyl 3-amino-6-(1-(1-((4-((2-(2,6-dioxopiperidin- |
| | 3-yl)-1,3-dioxoisoindolin-4-yl)glycyl)morpholin-2-yl)methyl)piperidin-4-yl)-1H-pyrazol-4- |
| | yl)pyrazine-2-carboxylate |
| TDH-307 | 2-((4-fluorophenyl)amino)-2-oxo-1-phenylethyl 3-amino-6-(1-(1-((4-((2-(2,6-dioxopiperidin- |
| | 3-yl)-1,3-dioxoisoindolin-5-yl)glycyl)morpholin-2-yl)methyl)piperidin-4-yl)-1H-pyrazol-4- |
| | yl)pyrazine-2-carboxylate |
| TDH-308 | 2-((4-fluorophenyl)amino)-2-oxo-1-phenylethyl 3-amino-6-(1-(1-((4-(2-((2-(2,6- |
| | dioxopiperidin-3-yl)-1,3-dioxoisoindolin-4-yl)oxy)acetyl)morpholin-2-yl)methyl)piperidin-4- |
| | yl)-1H-pyrazol-4-yl)pyrazine-2-carboxylate |
| TDH-309 | 2-((4-fluorophenyl)amino)-2-oxo-1-phenylethyl 3-amino-6-(1-(1-((4-(2-((2-(2,6- |
| | dioxopiperidin-3-yl)-1,3-dioxoisoindolin-5-yl)oxy)acetyl)morpholin-2-yl)methyl)piperidin-4- |
| | yl)-1H-pyrazol-4-yl)pyrazine-2-carboxylate |
| TDH-310 | 2-((4-fluorophenyl)amino)-2-oxo-1-phenylethyl 3-amino-6-(1-(1-((4-(2-(2,6-dioxopiperidin- |
| | 3-yl)-1,3-dioxoisoindolin-4-yl)morpholin-2-yl)methyl)piperidin-4-yl)-1H-pyrazol-4- |
| | yl)pyrazine-2-carboxylate |
| TDH-311 | 2-((4-fluorophenyl)amino)-2-oxo-1-phenylethyl 3-amino-6-(1-(1-((4-(2-(2,6-dioxopiperidin- |
| | 3-yl)-1,3-dioxoisoindolin-5-yl)morpholin-2-yl)methyl)piperidin-4-yl)-1H-pyrazol-4- |
| | yl)pyrazine-2-carboxylate |
| TDH-312 | 2-((4-fluorophenyl)amino)-2-oxo-1-phenylethyl 3-amino-6-(1-(1-(2-(1-(2-(((S)-1-((2R,4S)-4- |
| | hydroxy-2-((4-(4-methylthiazol-5-yl)benzyl)carbamoyl)pyrrolidin-1-yl)-3,3-dimethyl-1- |
| | oxobutan-2-yl)amino)-2-oxoethyl)piperidin-4-yl)ethyl)piperidin-4-yl)-1H-pyrazol-4- |
| | yl)pyrazine-2-carboxylate |
| TDH-328 | (R)-2-((4-fluorophenyl)amino)-2-oxo-1-phenylethyl 3-amino-6-(1-(1-((1-(3-(2-(2,6- |
| | dioxopiperidin-3-yl)-1,3-dioxoisoindolin-5-yl)propanoyl)piperidin-4-yl)methyl)piperidin-4-yl)- |
| | 1H-pyrazol-4-yl)pyrazine-2-carboxylate |
| TDH-330 | (R)-2-((4-fluorophenyl)amino)-2-oxo-1-phenylethyl 3-amino-6-(1-(1-(2-(1-(2-((3-(2,6- |
| | dioxopiperidin-3-yl)-4-oxo-3,4-dihydrobenzo[d][1,2,3]triazin-6-yl)oxy)acetyl)piperidin-4- |
| | yl)ethyl)piperidin-4-yl)-1H-pyrazol-4-yl)pyrazine-2-carboxylate |
| TDH-331 | (R)-2-((4-fluorophenyl)amino)-2-oxo-1-phenylethyl 3-amino-6-(1-(1-(2-((1-(2-((2-(2,6- |
| | dioxopiperidin-3-yl)-1,3-dioxoisoindolin-4-yl)oxy)acetyl)piperidin-4-yl)oxy)ethyl)piperidin-4- |
| | yl)-1H-pyrazol-4-yl)pyrazine-2-carboxylate |
| TDH-332 | (R)-2-((4-fluorophenyl)amino)-2-oxo-1-phenylethyl 3-amino-6-(1-(1-(2-((1-(2-((2-(2,6- |
| | dioxopiperidin-3-yl)-1,3-dioxoisoindolin-5-yl)oxy)acetyl)piperidin-4-yl)oxy)ethyl)piperidin-4- |
| | yl)-1H-pyrazol-4-yl)pyrazine-2-carboxylate |
| TDH-333 | (R)-2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)ethyl)piperidin-4-yl)-1H-pyrazol-4-yl)pyrazine-2-carboxylate |
| TDH-334 | (R)-2-((4-fluorophenyl)amino)-2-oxo-1-phenylethyl 3-amino-6-(1-(1-(2-(1-((3-(2,6- |
| | dioxopiperidin-3-yl)-4-oxo-3,4-dihydrobenzo[d][1,2,3]triazin-6-yl)glycyl)piperidin-4- |
| | yl)ethyl)piperidin-4-yl)-1H-pyrazol-4-yl)pyrazine-2-carboxylate |
| TDH-336 | (R)-2-((4-fluorophenyl)amino)-2-oxo-1-phenylethyl 3-amino-6-(1-(1-((1-(3-(2-(2,6- |
| | dioxopiperidin-3-yl)-1,3-dioxoisoindolin-4-yl)propanoyl)piperidin-4-yl)methyl)piperidin-4-yl)- |
| | 1H-pyrazol-4-yl)pyrazine-2-carboxylate |
| TDH-337 | (R)-2-((4-fluorophenyl)amino)-2-oxo-1-phenylethyl 3-amino-6-(1-(1-(2-(1-(2-((2-(2,6- |
| | dioxopiperidin-3-yl)-1,3-dioxoisoindolin-4-yl)oxy)acetyl)piperidin-4-yl)acetyl)piperidin-4-yl)- |
| | 1H-pyrazol-4-yl)pyrazine-2-carboxylate |
| TDH-338 | (R)-2-((4-fluorophenyl)amino)-2-oxo-1-phenylethyl 3-amino-6-(1-(1-(2-(1-(2-((2-(2,6- |
| | dioxopiperidin-3-yl)-1,3-dioxoisoindolin-5-yl)oxy)acetyl)piperidin-4-yl)acetyl)piperidin-4-yl)- |
| | 1H-pyrazol-4-yl)pyrazine-2-carboxylate |
| TDH-339 | (R)-2-((4-fluorophenyl)amino)-2-oxo-1-phenylethyl 3-amino-6-(1-(1-(2-(1-((2-(2,6- |
| | dioxopiperidin-3-yl)-1,3-dioxoisoindolin-4-yl)glycyl)piperidin-4-yl)acetyl)piperidin-4-yl)-1H- |
| | pyrazol-4-yl)pyrazine-2-carboxylate |
| TDH-340 | (R)-2-((4-fluorophenyl)amino)-2-oxo-1-phenylethyl 3-amino-6-(1-(1-(2-(1-((2-(2,6- |
| | dioxopiperidin-3-yl)-1,3-dioxoisoindolin-5-yl)glycyl)piperidin-4-yl)acetyl)piperidin-4-yl)-1H- |
| | pyrazol-4-yl)pyrazine-2-carboxylate |
| TDH-341 | (R)-2-((4-fluorophenyl)amino)-2-oxo-1-phenylethyl 3-amino-6-(1-(1-(2-(1-(1-(2-(2,6- |
| | dioxopiperidin-3-yl)-1,3-dioxoisoindolin-4-yl)piperidine-4-carbonyl)piperidin-4- |
| | yl)acetyl)piperidin-4-yl)-1H-pyrazol-4-yl)pyrazine-2-carboxylate |
| TDH-342 | (R)-2-((4-fluorophenyl)amino)-2-oxo-1-phenylethyl 3-amino-6-(1-(1-(2-(1-(1-(2-(2,6- |
| | dioxopiperidin-3-yl)-1,3-dioxoisoindolin-5-yl)piperidine-4-carbonyl)piperidin-4- |
| | yl)acetyl)piperidin-4-yl)-1H-pyrazol-4-yl)pyrazine-2-carboxylate |
| TDH-343 | (R)-2-((4-fluorophenyl)amino)-2-oxo-1-phenylethyl 3-amino-6-(1-(1-(2-(1-(2-(2,6- |
| | dioxopiperidin-3-yl)-1,3-dioxoisoindolin-4-yl)piperidin-4-yl)acetyl)piperidin-4-yl)-1H-pyrazol- |
| | 4-yl)pyrazine-2-carboxylate |
| TDH-344 | (R)-2-((4-fluorophenyl)amino)-2-oxo-1-phenylethyl 3-amino-6-(1-(1-(2-(1-(2-(2,6- |
| | dioxopiperidin-3-yl)-1,3-dioxoisoindolin-5-yl)piperidin-4-yl)acetyl)piperidin-4-yl)-1H-pyrazol- |
| | 4-yl)pyrazine-2-carboxylate |
| TDH-345 | (R)-2-((4-fluorophenyl)amino)-2-oxo-1-phenylethyl 3-amino-6-(1-(1-(((1r,4r)-4-((2-((2-(2,6- |
| | dioxopiperidin-3-yl)-1,3-dioxoisoindolin-4- |
| | yl)oxy)acetamido)methyl)cyclohexyl)methyl)piperidin-4-yl)-1H-pyrazol-4-yl)pyrazine-2- |
| | carboxylate |
| TDH-346 | (R)-2-((4-fluorophenyl)amino)-2-oxo-1-phenylethyl 3-amino-6-(1-(1-(((1r,4r)-4-((2-((2-(2,6- |
| | dioxopiperidin-3-yl)-1,3-dioxoisoindolin-5- |
| | yl)oxy)acetamido)methyl)cyclohexyl)methyl)piperidin-4-yl)-1H-pyrazol-4-yl)pyrazine-2- |
| | carboxylate |
| TDH-347 | (R)-2-((4-fluorophenyl)amino)-2-oxo-1-phenylethyl 3-amino-6-(1-(1-(((1r,4r)-4-((2-((2-(2,6- |
| | dioxopiperidin-3-yl)-1,3-dioxoisoindolin-4- |
| | yl)amino)acetamido)methyl)cyclohexyl)methyl)piperidin-4-yl)-1H-pyrazol-4-yl)pyrazine-2- |
| | carboxylate |
| TDH-348 | (R)-2-((4-fluorophenyl)amino)-2-oxo-1-phenylethyl 3-amino-6-(1-(1-(((1r,4r)-4-((2-((2-(2,6- |
| | dioxopiperidin-3-yl)-1,3-dioxoisoindolin-5- |
| | yl)amino)acetamido)methyl)cyclohexyl)methyl)piperidin-4-yl)-1H-pyrazol-4-yl)pyrazine-2- |
| | carboxylate |
| TDH-349 | (R)-2-((4-fluorophenyl)amino)-2-oxo-1-phenylethyl 3-amino-6-(1-(1-(((1r,4r)-4-((1-(2-(2,6- |
| | dioxopiperidin-3-yl)-1,3-dioxoisoindolin-4-yl)piperidine-4- |
| | carboxamido)methyl)cyclohexyl)methyl)piperidin-4-yl)-1H-pyrazol-4-yl)pyrazine-2- |
| | carboxylate |
| TDH-350 | (R)-2-((4-fluorophenyl)amino)-2-oxo-1-phenylethyl 3-amino-6-(1-(1-(((1r,4r)-4-((1-(2-(2,6- |
| | dioxopiperidin-3-yl)-1,3-dioxoisoindolin-5-yl)piperidine-4- |
| | carboxamido)methyl)cyclohexyl)methyl)piperidin-4-yl)-1H-pyrazol-4-yl)pyrazine-2- |
| | carboxylate |
| TDH-351 | (R)-2-((4-fluorophenyl)amino)-2-oxo-1-phenylethyl 3-amino-6-(1-(1-(((1r,4r)-4-(((2-(2,6- |
| | dioxopiperidin-3-yl)-1,3-dioxoisoindolin-4-yl)amino)methyl)cyclohexyl)methyl)piperidin-4- |
| | yl)-1H-pyrazol-4-yl)pyrazine-2-carboxylate |
| TDH-352 | (R)-2-((4-fluorophenyl)amino)-2-oxo-1-phenylethyl 3-amino-6-(1-(1-(((1r,4r)-4-(((2-(2,6- |
| | dioxopiperidin-3-yl)-1,3-dioxoisoindolin-5-yl)amino)methyl)cyclohexyl)methyl)piperidin-4- |
| | yl)-1H-pyrazol-4-yl)pyrazine-2-carboxylate |
| TDH-353 | (R)-2-((4-fluorophenyl)amino)-2-oxo-1-phenylethyl 3-amino-6-(1-(1-(1-((2-(2,6- |
| | dioxopiperidin-3-yl)-1,3-dioxoisoindolin-5-yl)glycyl)piperidine-4-carbonyl)piperidin-4-yl)-1H- |
| | pyrazol-4-yl)pyrazine-2-carboxylate |
| TDH-354 | (R)-2-((4-fluorophenyl)amino)-2-oxo-1-phenylethyl 3-amino-6-(1-(1-(1-(1-(2-((2-(2,6- |
| | dioxopiperidin-3-yl)-1,3-dioxoisoindolin-4-yl)oxy)acetyl)piperidin-4-yl)-1H-imidazole-4- |
| | carbonyl)piperidin-4-yl)-1H-pyrazol-4-yl)pyrazine-2-carboxylate |
| TDH-355 | (R)-2-((4-fluorophenyl)amino)-2-oxo-1-phenylethyl 3-amino-6-(1-(1-(1-(1-(2-((2-(2,6- |
| | dioxopiperidin-3-yl)-1,3-dioxoisoindolin-5-yl)oxy)acetyl)piperidin-4-yl)-1H-imidazole-4- |
| | carbonyl)piperidin-4-yl)-1H-pyrazol-4-yl)pyrazine-2-carboxylate |
| TDH-356 | (R)-2-((4-fluorophenyl)amino)-2-oxo-1-phenylethyl 3-amino-6-(1-(1-(1-(1-((2-(2,6- |
| | dioxopiperidin-3-yl)-1,3-dioxoisoindolin-4-yl)glycyl)piperidin-4-yl)-1H-imidazole-4- |
| | carbonyl)piperidin-4-yl)-1H-pyrazol-4-yl)pyrazine-2-carboxylate |
| TDH-357 | (R)-2-((4-fluorophenyl)amino)-2-oxo-1-phenylethyl 3-amino-6-(1-(1-(3-(4-((2-(2,6- |
| | dioxopiperidin-3-yl)-1,3-dioxoisoindolin-5-yl)glycyl)piperazin-1-yl)propyl)piperidin-4-yl)-1H- |
| | pyrazol-4-yl)pyrazine-2-carboxylate |
| TDH-358 | (R)-2-((4-fluorophenyl)amino)-2-oxo-1-phenylethyl 3-amino-6-(1-(1-(3-(4-(2-((2-(2,6- |
| | dioxopiperidin-3-yl)-1,3-dioxoisoindolin-5-yl)oxy)acetyl)piperazin-1-yl)propyl)piperidin-4- |
| | yl)-1H-pyrazol-4-yl)pyrazine-2-carboxylate |
| TDH-359 | (R)-2-((4-fluorophenyl)amino)-2-oxo-1-phenylethyl 3-amino-6-(1-((3R)-1-((1-(2-((2-(2,6- |
| | dioxopiperidin-3-yl)-1,3-dioxoisoindolin-5-yl)oxy)acetyl)piperidin-4-yl)methyl)pyrrolidin-3- |
| | yl)-1H-pyrazol-4-yl)pyrazine-2-carboxylate |
| TDH-360 | (R)-2-((4-fluorophenyl)amino)-2-oxo-1-phenylethyl 3-amino-6-(1-(1-((1-((2-(2,6- |
| | dioxopiperidin-3-yl)-1,3-dioxoisoindolin-5-yl)glycyl)piperidin-4-yl)methyl)azetidin-3-yl)-1H- |
| | pyrazol-4-yl)pyrazine-2-carboxylate |
| TDH-361 | (R)-2-((4-fluorophenyl)amino)-2-oxo-1-phenylethyl 3-amino-6-(1-(1-(1-(1-((2-(2,6- |
| | dioxopiperidin-3-yl)-1,3-dioxoisoindolin-5-yl)glycyl)piperidin-4-yl)-1H-imidazole-4- |
| | carbonyl)piperidin-4-yl)-1H-pyrazol-4-yl)pyrazine-2-carboxylate |
| TDH-362 | (R)-2-((4-fluorophenyl)amino)-2-oxo-1-phenylethyl 3-amino-6-(1-(1-(1-(1-(1-(2-(2,6- |
| | dioxopiperidin-3-yl)-1,3-dioxoisoindolin-4-yl)piperidine-4-carbonyl)piperidin-4-yl)-1H- |
| | imidazole-4-carbonyl)piperidin-4-yl)-1H-pyrazol-4-yl)pyrazine-2-carboxylate |
| TDH-363 | (R)-2-((4-fluorophenyl)amino)-2-oxo-1-phenylethyl 3-amino-6-(1-(1-(1-(1-(1-(2-(2,6- |
| | dioxopiperidin-3-yl)-1,3-dioxoisoindolin-5-yl)piperidine-4-carbonyl)piperidin-4-yl)-1H- |
| | imidazole-4-carbonyl)piperidin-4-yl)-1H-pyrazol-4-yl)pyrazine-2-carboxylate |
| TDH-364 | (R)-2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-4-yl)piperidin-4-yl)-1H-imidazole-4- |
| | carbonyl)piperidin-4-yl)-1H-pyrazol-4-yl)pyrazine-2-carboxylate |
| TDH-365 | (R)-2-((4-fluorophenyl)amino)-2-oxo-1-phenylethyl 3-amino-6-(1-(1-(1-(1-(2-(2,6- |
| | dioxopiperidin-3-yl)-1,3-dioxoisoindolin-5-yl)piperidin-4-yl)-1H-imidazole-4- |
| | carbonyl)piperidin-4-yl)-1H-pyrazol-4-yl)pyrazine-2-carboxylate |
| TDH-366 | (R)-2-((4-fluorophenyl)amino)-2-oxo-1-phenylethyl 3-amino-6-(1-(1-(2-(1-(2-(1-(2-(2,6- |
| | dioxopiperidin-3-yl)-1,3-dioxoisoindolin-4-yl)piperidin-4-yl)acetyl)piperidin-4- |
| | yl)ethyl)piperidin-4-yl)-1H-pyrazol-4-yl)pyrazine-2-carboxylate |
| TDH-367 | (R)-2-((4-fluorophenyl)amino)-2-oxo-1-phenylethyl 3-amino-6-(1-(1-(2-(1-(2-(1-(2-(2,6- |
| | dioxopiperidin-3-yl)-1,3-dioxoisoindolin-5-yl)piperidin-4-yl)acetyl)piperidin-4- |
| | yl)ethyl)piperidin-4-yl)-1H-pyrazol-4-yl)pyrazine-2-carboxylate |
| TDH-368 | (R)-2-((4-fluorophenyl)amino)-2-oxo-1-phenylethyl 3-amino-6-(1-(1-(3-(4-(2-(1-(2-(2,6- |
| | dioxopiperidin-3-yl)-1,3-dioxoisoindolin-4-yl)piperidin-4-yl)acetyl)piperazin-1- |
| | yl)propyl)piperidin-4-yl)-1H-pyrazol-4-yl)pyrazine-2-carboxylate |
| TDH-369 | (R)-2-((4-fluorophenyl)amino)-2-oxo-1-phenylethyl 3-amino-6-(1-(1-(3-(4-(2-(1-(2-(2,6- |
| | dioxopiperidin-3-yl)-1,3-dioxoisoindolin-5-yl)piperidin-4-yl)acetyl)piperazin-1- |
| | yl)propyl)piperidin-4-yl)-1H-pyrazol-4-yl)pyrazine-2-carboxylate |
| TDH-370 | (R)-2-((4-fluorophenyl)amino)-2-oxo-1-phenylethyl 3-amino-6-(1-(1-(3-(4-(2-((3-(2,6- |
| | dioxopiperidin-3-yl)-4-oxo-3,4-dihydrobenzo[d][1,2,3]triazin-6-yl)oxy)acetyl)piperazin-1- |
| | yl)propyl)piperidin-4-yl)-1H-pyrazol-4-yl)pyrazine-2-carboxylate |
| TDH-371 | (R)-2-((4-fluorophenyl)amino)-2-oxo-1-phenylethyl 3-amino-6-(1-(1-(2-((1-(2-(1-(2-(2,6- |
| | dioxopiperidin-3-yl)-1,3-dioxoisoindolin-4-yl)piperidin-4-yl)acetyl)piperidin-4- |
| | yl)oxy)ethyl)piperidin-4-yl)-1H-pyrazol-4-yl)pyrazine-2-carboxylate |
| TDH-372 | (R)-2-((4-fluorophenyl)amino)-2-oxo-1-phenylethyl 3-amino-6-(1-(1-(2-((1-(2-(1-(2-(2,6- |
| | dioxopiperidin-3-yl)-1,3-dioxoisoindolin-5-yl)piperidin-4-yl)acetyl)piperidin-4- |
| | yl)oxy)ethyl)piperidin-4-yl)-1H-pyrazol-4-yl)pyrazine-2-carboxylate |
| TDH-373 | (R)-2-((4-fluorophenyl)amino)-2-oxo-1-phenylethyl 3-amino-6-(1-(1-(2-((1-(2-((3-(2,6- |
| | dioxopiperidin-3-yl)-4-oxo-3,4-dihydrobenzo[d][1,2,3]triazin-6-yl)oxy)acetyl)piperidin-4- |
| | yl)oxy)ethyl)piperidin-4-yl)-1H-pyrazol-4-yl)pyrazine-2-carboxylate |
| TDH-374 | (R)-2-((4-fluorophenyl)amino)-2-oxo-1-phenylethyl 3-amino-6-(1-(1-(1-(2-(1-(2-(2,6- |
| | dioxopiperidin-3-yl)-1,3-dioxoisoindolin-4-yl)piperidin-4-yl)acetyl)piperidine-4- |
| | carbonyl)piperidin-4-yl)-1H-pyrazol-4-yl)pyrazine-2-carboxylate |
| TDH-375 | (R)-2-((4-fluorophenyl)amino)-2-oxo-1-phenylethyl 3-amino-6-(1-(1-(1-(2-(1-(2-(2,6- |
| | dioxopiperidin-3-yl)-1,3-dioxoisoindolin-5-yl)piperidin-4-yl)acetyl)piperidine-4- |
| | carbonyl)piperidin-4-yl)-1H-pyrazol-4-yl)pyrazine-2-carboxylate |
| TDH-376 | (R)-2-((4-fluorophenyl)amino)-2-oxo-1-phenylethyl 3-amino-6-(1-(1-(2-(1-(3-(2-(2,6- |
| | dioxopiperidin-3-yl)-1,3-dioxoisoindolin-4-yl)propanoyl)piperidin-4-yl)ethyl)piperidin-4-yl)- |
| | 1H-pyrazol-4-yl)pyrazine-2-carboxylate |
| TDH-377 | (R)-2-((4-fluorophenyl)amino)-2-oxo-1-phenylethyl 3-amino-6-(1-(1-(2-(1-(3-(2-(2,6- |
| | dioxopiperidin-3-yl)-1,3-dioxoisoindolin-5-yl)propanoyl)piperidin-4-yl)ethyl)piperidin-4-yl)- |
| | 1H-pyrazol-4-yl)pyrazine-2-carboxylate |
| TDH-378 | (R)-2-((4-fluorophenyl)amino)-2-oxo-1-phenylethyl 3-amino-6-(1-(1-(2-((1-(3-(2-(2,6- |
| | dioxopiperidin-3-yl)-1,3-dioxoisoindolin-4-yl)propanoyl)piperidin-4-yl)oxy)ethyl)piperidin-4- |
| | yl)-1H-pyrazol-4-yl)pyrazine-2-carboxylate |
| TDH-379 | (R)-2-((4-fluorophenyl)amino)-2-oxo-1-phenylethyl 3-amino-6-(1-(1-(2-((1-(3-(2-(2,6- |
| | dioxopiperidin-3-yl)-1,3-dioxoisoindolin-5-yl)propanoyl)piperidin-4-yl)oxy)ethyl)piperidin-4- |
| | yl)-1H-pyrazol-4-yl)pyrazine-2-carboxylate |
| TDH-380 | (R)-2-((4-fluorophenyl)amino)-2-oxo-1-phenylethyl 3-amino-6-(1-(1-(1-(3-(2-(2,6- |
| | dioxopiperidin-3-yl)-1,3-dioxoisoindolin-4-yl)propanoyl)piperidine-4-carbonyl)piperidin-4- |
| | yl)-1H-pyrazol-4-yl)pyrazine-2-carboxylate |
| TDH-381 | (R)-2-((4-fluorophenyl)amino)-2-oxo-1-phenylethyl 3-amino-6-(1-(1-(1-(3-(2-(2,6- |
| | dioxopiperidin-3-yl)-1,3-dioxoisoindolin-5-yl)propanoyl)piperidine-4-carbonyl)piperidin-4- |
| | yl)-1H-pyrazol-4-yl)pyrazine-2-carboxylate |
| TDH-382 | (R)-2-((4-fluorophenyl)amino)-2-oxo-1-phenylethyl 3-amino-6-(1-(1-(3-(4-(3-(2-(2,6- |
| | dioxopiperidin-3-yl)-1,3-dioxoisoindolin-4-yl)propanoyl)piperazin-1-yl)propyl)piperidin-4-yl)- |
| | 1H-pyrazol-4-yl)pyrazine-2-carboxylate |
| TDH-383 | (R)-2-((4-fluorophenyl)amino)-2-oxo-1-phenylethyl 3-amino-6-(1-(1-(3-(4-(3-(2-(2,6- |
| | dioxopiperidin-3-yl)-1,3-dioxoisoindolin-5-yl)propanoyl)piperazin-1-yl)propyl)piperidin-4-yl)- |
| | 1H-pyrazol-4-yl)pyrazine-2-carboxylate |
| TDH-384 | (R)-2-((4-fluorophenyl)amino)-2-oxo-1-phenylethyl 3-amino-6-(1-(1-((1-(3-(2-(2,6- |
| | dioxopiperidin-3-yl)-1-oxoisoindolin-4-yl)propanoyl)piperidin-4-yl)methyl)piperidin-4-yl)-1H- |
| | pyrazol-4-yl)pyrazine-2-carboxylate |
| TDH-385 | (R)-2-((4-fluorophenyl)amino)-2-oxo-1-phenylethyl 3-amino-6-(1-(1-(2-(1-(3-(2-(2,6- |
| | dioxopiperidin-3-yl)-1-oxoisoindolin-4-yl)propanoyl)piperidin-4-yl)ethyl)piperidin-4-yl)-1H- |
| | pyrazol-4-yl)pyrazine-2-carboxylate |
| TDH-386 | (R)-2-((4-fluorophenyl)amino)-2-oxo-1-phenylethyl 3-amino-6-(1-(1-(3-(4-(3-(2-(2,6- |
| | dioxopiperidin-3-yl)-1-oxoisoindolin-4-yl)propanoyl)piperazin-1-yl)propyl)piperidin-4-yl)-1H- |
| | pyrazol-4-yl)pyrazine-2-carboxylate |
| TDH-387 | (R)-2-((4-fluorophenyl)amino)-2-oxo-1-phenylethyl 3-amino-6-(1-(1-(2-((1-(3-(2-(2,6- |
| | dioxopiperidin-3-yl)-1-oxoisoindolin-4-yl)propanoyl)piperidin-4-yl)oxy)ethyl)piperidin-4-yl)- |
| | 1H-pyrazol-4-yl)pyrazine-2-carboxylate |
| TDH-388 | (R)-2-((4-fluorophenyl)amino)-2-oxo-1-phenylethyl 3-amino-6-(1-(1-(1-(3-(2-(2,6- |
| | dioxopiperidin-3-yl)-1-oxoisoindolin-4-yl)propanoyl)piperidine-4-carbonyl)piperidin-4-yl)- |
| | 1H-pyrazol-4-yl)pyrazine-2-carboxylate |
| TDH-389 | (R)-2-((4-fluorophenyl)amino)-2-oxo-1-phenylethyl 3-amino-6-(1-(1-(3-(4-(2-((2-(2,6- |
| | dioxopiperidin-3-yl)-1,3-dioxoisoindolin-5-yl)oxy)acetyl)piperazin-1-yl)propyl)piperidin-4- |
| | yl)-1H-pyrazol-4-yl)pyrazine-2-carboxylate hydrochloride |
| TDH-390 | (R)-2-((4-fluorophenyl)amino)-2-oxo-1-phenylethyl 3-amino-6-(1-(1-((4-(2-((2-(2,6- |
| | dioxopiperidin-3-yl)-1,3-dioxoisoindolin-5-yl)oxy)acetyl)morpholin-2-yl)methyl)piperidin-4- |
| | yl)-1H-pyrazol-4-yl)pyrazine-2-carboxylate |
| TDH-391 | (R)-2-((4-fluorophenyl)amino)-2-oxo-1-phenylethyl 3-amino-6-(1-(1-(2-(1-((2-(2,6- |
| | dioxopiperidin-3-yl)-1,3-dioxoisoindolin-4-yl)glycyl)piperidin-4-yl)ethyl)piperidin-4-yl)-1H- |
| | pyrazol-4-yl)pyrazine-2-carboxylate hydrochloride |
| TDH-392 | (R)-2-((4-fluorophenyl)amino)-2-oxo-1-phenylethyl 3-amino-6-(1-(1-(2-(1-(2-((2-(2,6- |
| | dioxopiperidin-3-yl)-1,3-dioxoisoindolin-5-yl)oxy)acetyl)piperidin-4-yl)ethyl)piperidin-4-yl)- |
| | 1H-pyrazol-4-yl)pyrazine-2-carboxylate hydrochloride |
| TDH-393 | (R)-2-((4-fluorophenyl)amino)-2-oxo-1-phenylethyl 3-amino-6-(1-(1-(2-((1-(2-((2-(2,6- |
| | dioxopiperidin-3-yl)-1,3-dioxoisoindolin-4-yl)oxy)acetyl)piperidin-4-yl)oxy)acetyl)piperidin- |
| | 4-yl)-1H-pyrazol-4-yl)pyrazine-2-carboxylate |
| TDH-394 | (R)-2-((4-fluorophenyl)amino)-2-oxo-1-phenylethyl 3-amino-6-(1-(1-(2-((1-(2-((2-(2,6- |
| | dioxopiperidin-3-yl)-1,3-dioxoisoindolin-5-yl)oxy)acetyl)piperidin-4-yl)oxy)acetyl)piperidin- |
| | 4-yl)-1H-pyrazol-4-yl)pyrazine-2-carboxylate |
| TDH-395 | (R)-2-((4-fluorophenyl)amino)-2-oxo-1-phenylethyl 3-amino-6-(1-(1-(2-((1-((2-(2,6- |
| | dioxopiperidin-3-yl)-1,3-dioxoisoindolin-4-yl)glycyl)piperidin-4-yl)oxy)acetyl)piperidin-4-yl)- |
| | 1H-pyrazol-4-yl)pyrazine-2-carboxylate |
| TDH-396 | (R)-2-((4-fluorophenyl)amino)-2-oxo-1-phenylethyl 3-amino-6-(1-(1-(2-((1-((2-(2,6- |
| | dioxopiperidin-3-yl)-1,3-dioxoisoindolin-5-yl)glycyl)piperidin-4-yl)oxy)acetyl)piperidin-4-yl)- |
| | 1H-pyrazol-4-yl)pyrazine-2-carboxylate |
| TDH-397 | (R)-2-((4-fluorophenyl)amino)-2-oxo-1-phenylethyl 3-amino-6-(1-(1-(2-((1-(1-(2-(2,6- |
| | dioxopiperidin-3-yl)-1,3-dioxoisoindolin-4-yl)piperidine-4-carbonyl)piperidin-4- |
| | yl)oxy)acetyl)piperidin-4-yl)-1H-pyrazol-4-yl)pyrazine-2-carboxylate |
| TDH-398 | (R)-2-((4-fluorophenyl)amino)-2-oxo-1-phenylethyl 3-amino-6-(1-(1-(2-((1-(1-(2-(2,6- |
| | dioxopiperidin-3-yl)-1,3-dioxoisoindolin-5-yl)piperidine-4-carbonyl)piperidin-4- |
| | yl)oxy)acetyl)piperidin-4-yl)-1H-pyrazol-4-yl)pyrazine-2-carboxylate |
| TDH-399 | (R)-2-((4-fluorophenyl)amino)-2-oxo-1-phenylethyl 3-amino-6-(1-(1-(2-((1-(2-(2,6- |
| | dioxopiperidin-3-yl)-1,3-dioxoisoindolin-4-yl)piperidin-4-yl)oxy)acetyl)piperidin-4-yl)-1H- |
| | pyrazol-4-yl)pyrazine-2-carboxylate |
| TDH-400 | (R)-2-((4-fluorophenyl)amino)-2-oxo-1-phenylethyl 3-amino-6-(1-(1-(2-((1-(2-(2,6- |
| | dioxopiperidin-3-yl)-1,3-dioxoisoindolin-5-yl)piperidin-4-yl)oxy)acetyl)piperidin-4-yl)-1H- |
| | pyrazol-4-yl)pyrazine-2-carboxylate |
| TDH-401 | (R)-2-((4-fluorophenyl)amino)-2-oxo-1-phenylethyl 3-amino-6-(1-(1-(2-(1-(1-(2-(2,6- |
| | dioxopiperidin-3-yl)-1,3-dioxoisoindolin-5-yl)azetidine-3-carbonyl)piperidin-4- |
| | yl)acetyl)piperidin-4-yl)-1H-pyrazol-4-yl)pyrazine-2-carboxylate |
| TDH-402 | (R)-2-((4-fluorophenyl)amino)-2-oxo-1-phenylethyl 3-amino-6-(1-(1-(2-(1-(2-(1-(2-(2,6- |
| | dioxopiperidin-3-yl)-1,3-dioxoisoindolin-5-yl)piperidin-4-yl)acetyl)piperidin-4- |
| | yl)acetyl)piperidin-4-yl)-1H-pyrazol-4-yl)pyrazine-2-carboxylate |
| TDH-403 | (R)-2-((4-fluorophenyl)amino)-2-oxo-1-phenylethyl 3-amino-6-(1-(1-(2-(1-(3-(2-(2,6- |
| | dioxopiperidin-3-yl)-1,3-dioxoisoindolin-5-yl)propanoyl)piperidin-4-yl)acetyl)piperidin-4-yl)- |
| | 1H-pyrazol-4-yl)pyrazine-2-carboxylate |
| TDH-404 | (R)-2-((4-fluorophenyl)amino)-2-oxo-1-phenylethyl 3-amino-6-(1-(1-(2-(1-(3-(2-(2,6- |
| | dioxopiperidin-3-yl)-1-oxoisoindolin-4-yl)propanoyl)piperidin-4-yl)acetyl)piperidin-4-yl)-1H- |
| | pyrazol-4-yl)pyrazine-2-carboxylate |
| TDH-405 | (R)-2-((4-fluorophenyl)amino)-2-oxo-1-phenylethyl 3-amino-6-(1-(1-(2-(1-((3-(2,6- |
| | dioxopiperidin-3-yl)-4-oxo-3,4-dihydrobenzo[d][1,2,3]triazin-6-yl)glycyl)piperidin-4- |
| | yl)acetyl)piperidin-4-yl)-1H-pyrazol-4-yl)pyrazine-2-carboxylate |
| TDH-406 | (R)-2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)acetyl)piperidin-4-yl)-1H-pyrazol-4-yl)pyrazine-2-carboxylate |
| TDH-407 | (R)-2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)acetyl)piperidin-4-yl)-1H-pyrazol-4-yl)pyrazine-2-carboxylate |
| TDH-408 | (R)-2-((4-fluorophenyl)amino)-2-oxo-1-phenylethyl 3-amino-6-(1-(1-(1-((3-(2,6- |
| | dioxopiperidin-3-yl)-4-oxo-3,4-dihydrobenzo[d][1,2,3]triazin-6-yl)glycyl)piperidine-4- |
| | carbonyl)piperidin-4-yl)-1H-pyrazol-4-yl)pyrazine-2-carboxylate |
| TDH-409 | (R)-2-((4-fluorophenyl)amino)-2-oxo-1-phenylethyl 3-amino-6-(1-(1-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidine-4-carbonyl)piperidin-4-yl)-1H-pyrazol-4-yl)pyrazine-2-carboxylate |
| TDH-410 | (R)-2-((4-fluorophenyl)amino)-2-oxo-1-phenylethyl 3-amino-6-(1-(1-(1-(1-(3-(2,6- |
| | dioxopiperidin-3-yl)-4-oxo-3,4-dihydrobenzo[d][1,2,3]triazin-6-yl)piperidine-4- |
| | carbonyl)piperidine-4-carbonyl)piperidin-4-yl)-1H-pyrazol-4-yl)pyrazine-2-carboxylate |
| TDH-411 | (R)-2-((4-fluorophenyl)amino)-2-oxo-1-phenylethyl 3-amino-6-(1-(1-(2-(1-((3-(2,6- |
| | dioxopiperidin-3-yl)-4-oxo-3,4-dihydrobenzo[d][1,2,3]triazin-5-yl)glycyl)piperidin-4- |
| | yl)acetyl)piperidin-4-yl)-1H-pyrazol-4-yl)pyrazine-2-carboxylate |
| TDH-412 | (R)-2-((4-fluorophenyl)amino)-2-oxo-1-phenylethyl 3-amino-6-(1-(1-(2-(1-(1-(3-(2,6- |
| | dioxopiperidin-3-yl)-4-oxo-3,4-dihydrobenzo[d][1,2,3]triazin-5-yl)piperidine-4- |
| | carbonyl)piperidin-4-yl)acetyl)piperidin-4-yl)-1H-pyrazol-4-yl)pyrazine-2-carboxylate |
| TDH-413 | (R)-2-((4-fluorophenyl)amino)-2-oxo-1-phenylethyl 3-amino-6-(1-(1-(1-((3-(2,6- |
| | dioxopiperidin-3-yl)-4-oxo-3,4-dihydrobenzo[d][1,2,3]triazin-5-yl)glycyl)piperidine-4- |
| | carbonyl)piperidin-4-yl)-1H-pyrazol-4-yl)pyrazine-2-carboxylate |
| TDH-414 | (R)-2-((4-fluorophenyl)amino)-2-oxo-1-phenylethyl 3-amino-6-(1-(1-(1-(1-(3-(2,6- |
| | dioxopiperidin-3-yl)-4-oxo-3,4-dihydrobenzo[d][1,2,3]triazin-5-yl)piperidine-4- |
| | carbonyl)piperidine-4-carbonyl)piperidin-4-yl)-1H-pyrazol-4-yl)pyrazine-2-carboxylate |
| TDH-415 | (R)-2-((4-fluorophenyl)amino)-2-oxo-1-phenylethyl 3-amino-6-(1-(1-(3-(1-(2-((2-(2,6- |
| | dioxopiperidin-3-yl)-1,3-dioxoisoindolin-4-yl)oxy)acetyl)piperidin-4-yl)propanoyl)piperidin- |
| | 4-yl)-1H-pyrazol-4-yl)pyrazine-2-carboxylate |
| TDH-416 | (R)-2-((4-fluorophenyl)amino)-2-oxo-1-phenylethyl 3-amino-6-(1-(1-(3-(1-(2-((2-(2,6- |
| | dioxopiperidin-3-yl)-1,3-dioxoisoindolin-5-yl)oxy)acetyl)piperidin-4-yl)propanoyl)piperidin- |
| | 4-yl)-1H-pyrazol-4-yl)pyrazine-2-carboxylate |
| TDH-417 | (R)-2-((4-fluorophenyl)amino)-2-oxo-1-phenylethyl 3-amino-6-(1-(1-(3-(1-(1-(2-(2,6- |
| | dioxopiperidin-3-yl)-1,3-dioxoisoindolin-4-yl)piperidine-4-carbonyl)piperidin-4- |
| | yl)propanoyl)piperidin-4-yl)-1H-pyrazol-4-yl)pyrazine-2-carboxylate |
| TDH-418 | (R)-2-((4-fluorophenyl)amino)-2-oxo-1-phenylethyl 3-amino-6-(1-(1-(3-(1-(1-(2-(2,6- |
| | dioxopiperidin-3-yl)-1,3-dioxoisoindolin-5-yl)piperidine-4-carbonyl)piperidin-4- |
| | yl)propanoyl)piperidin-4-yl)-1H-pyrazol-4-yl)pyrazine-2-carboxylate |
| TDH-419 | (R)-2-((4-fluorophenyl)amino)-2-oxo-1-phenylethyl 3-amino-6-(1-(1-(3-(1-(2-(2,6- |
| | dioxopiperidin-3-yl)-1,3-dioxoisoindolin-4-yl)piperidin-4-yl)propanoyl)piperidin-4-yl)-1H- |
| | pyrazol-4-yl)pyrazine-2-carboxylate |
| TDH-420 | (R)-2-((4-fluorophenyl)amino)-2-oxo-1-phenylethyl 3-amino-6-(1-(1-(3-(1-(2-(2,6- |
| | dioxopiperidin-3-yl)-1,3-dioxoisoindolin-5-yl)piperidin-4-yl)propanoyl)piperidin-4-yl)-1H- |
| | pyrazol-4-yl)pyrazine-2-carboxylate |
| TDH-421 | (R)-2-((4-fluorophenyl)amino)-2-oxo-1-phenylethyl 3-amino-6-(1-(1-(3-(1-((2-(2,6- |
| | dioxopiperidin-3-yl)-1,3-dioxoisoindolin-4-yl)glycyl)piperidin-4-yl)propanoyl)piperidin-4-yl)- |
| | 1H-pyrazol-4-yl)pyrazine-2-carboxylate |
| TDH-422 | (R)-2-((4-fluorophenyl)amino)-2-oxo-1-phenylethyl 3-amino-6-(1-(1-(3-(1-((2-(2,6- |
| | dioxopiperidin-3-yl)-1,3-dioxoisoindolin-5-yl)glycyl)piperidin-4-yl)propanoyl)piperidin-4-yl)- |
| | 1H-pyrazol-4-yl)pyrazine-2-carboxylate |
| TDH-423 | (R)-2-((4-fluorophenyl)amino)-2-oxo-1-phenylethyl 3-amino-6-(1-(1-(8-(2-(2,6- |
| | dioxopiperidin-3-yl)-1-oxoisoindolin-4-yl)oct-7-ynoyl)piperidin-4-yl)-1H-pyrazol-4- |
| | yl)pyrazine-2-carboxylate |
| TDH-425 | (R)-2-((4-fluorophenyl)amino)-2-oxo-1-phenylethyl 3-amino-6-(1-(1-((1-((1-(2-((2-(2,6- |
| | dioxopiperidin-3-yl)-1,3-dioxoisoindolin-5-yl)oxy)acetyl)piperidin-4-yl)methyl)piperidin-4- |
| | yl)methyl)piperidin-4-yl)-1H-pyrazol-4-yl)pyrazine-2-carboxylate |
| TDH-426 | (R)-2-((4-fluorophenyl)amino)-2-oxo-1-phenylethyl 3-amino-6-(1-(1-((1-((1-((2-(2,6- |
| | dioxopiperidin-3-yl)-1,3-dioxoisoindolin-5-yl)glycyl)piperidin-4-yl)methyl)piperidin-4- |
| | yl)methyl)piperidin-4-yl)-1H-pyrazol-4-yl)pyrazine-2-carboxylate |
| TDH-427 | (R)-2-((4-fluorophenyl)amino)-2-oxo-1-phenylethyl 3-amino-6-(1-(1-(3-(1-(1-(2-(2,6- |
| | dioxopiperidin-3-yl)-1,3-dioxoisoindolin-4-yl)azetidine-3-carbonyl)piperidin-4- |
| | yl)propanoyl)piperidin-4-yl)-1H-pyrazol-4-yl)pyrazine-2-carboxylate |
| TDH-428 | (R)-2-((4-fluorophenyl)amino)-2-oxo-1-phenylethyl 3-amino-6-(1-(1-(3-(1-(1-(2-(2,6- |
| | dioxopiperidin-3-yl)-1,3-dioxoisoindolin-5-yl)azetidine-3-carbonyl)piperidin-4- |
| | yl)propanoyl)piperidin-4-yl)-1H-pyrazol-4-yl)pyrazine-2-carboxylate |
| TDH-429 | (R)-2-((4-fluorophenyl)amino)-2-oxo-1-phenylethyl 3-amino-6-(1-(1-(3-(1-(2-(1-(2-(2,6- |
| | dioxopiperidin-3-yl)-1,3-dioxoisoindolin-4-yl)piperidin-4-yl)acetyl)piperidin-4- |
| | yl)propanoyl)piperidin-4-yl)-1H-pyrazol-4-yl)pyrazine-2-carboxylate |
| TDH-430 | (R)-2-((4-fluorophenyl)amino)-2-oxo-1-phenylethyl 3-amino-6-(1-(1-(3-(1-(2-(1-(2-(2,6- |
| | dioxopiperidin-3-yl)-1,3-dioxoisoindolin-5-yl)piperidin-4-yl)acetyl)piperidin-4- |
| | yl)propanoyl)piperidin-4-yl)-1H-pyrazol-4-yl)pyrazine-2-carboxylate |
| TDH-431 | (R)-2-((4-fluorophenyl)amino)-2-oxo-1-phenylethyl 3-amino-6-(1-(1-(3-(1-(3-(2-(2,6- |
| | dioxopiperidin-3-yl)-1,3-dioxoisoindolin-4-yl)propanoyl)piperidin-4-yl)propanoyl)piperidin-4- |
| | yl)-1H-pyrazol-4-yl)pyrazine-2-carboxylate |
| TDH-432 | (R)-2-((4-fluorophenyl)amino)-2-oxo-1-phenylethyl 3-amino-6-(1-(1-(3-(1-(3-(2-(2,6- |
| | dioxopiperidin-3-yl)-1,3-dioxoisoindolin-5-yl)propanoyl)piperidin-4-yl)propanoyl)piperidin-4- |
| | yl)-1H-pyrazol-4-yl)pyrazine-2-carboxylate |
| TDH-433 | (R)-2-((4-fluorophenyl)amino)-2-oxo-1-phenylethyl 3-amino-6-(1-(1-(3-(1-((3-(2,6- |
| | dioxopiperidin-3-yl)-4-oxo-3,4-dihydrobenzo[d][1,2,3]triazin-5-yl)glycyl)piperidin-4- |
| | yl)propanoyl)piperidin-4-yl)-1H-pyrazol-4-yl)pyrazine-2-carboxylate |
| TDH-434 | (R)-2-((4-fluorophenyl)amino)-2-oxo-1-phenylethyl 3-amino-6-(1-(1-(3-(1-((3-(2,6- |
| | dioxopiperidin-3-yl)-4-oxo-3,4-dihydrobenzo[d][1,2,3]triazin-6-yl)glycyl)piperidin-4- |
| | yl)propanoyl)piperidin-4-yl)-1H-pyrazol-4-yl)pyrazine-2-carboxylate |
| TDH-435 | (R)-2-((4-fluorophenyl)amino)-2-oxo-1-phenylethyl 3-amino-6-(1-(1-(3-(1-(1-(3-(2,6- |
| | dioxopiperidin-3-yl)-4-oxo-3,4-dihydrobenzo[d][1,2,3]triazin-5-yl)piperidine-4- |
| | carbonyl)piperidin-4-yl)propanoyl)piperidin-4-yl)-1H-pyrazol-4-yl)pyrazine-2-carboxylate |
| TDH-436 | (R)-2-((4-fluorophenyl)amino)-2-oxo-1-phenylethyl 3-amino-6-(1-(1-(3-(1-(1-(3-(2,6- |
| | dioxopiperidin-3-yl)-4-oxo-3,4-dihydrobenzo[d][1,2,3]triazin-6-yl)piperidine-4- |
| | carbonyl)piperidin-4-yl)propanoyl)piperidin-4-yl)-1H-pyrazol-4-yl)pyrazine-2-carboxylate |
| TDH-437 | (R)-2-((4-fluorophenyl)amino)-2-oxo-1-phenylethyl 3-amino-6-(1-(1-(3-(1-(2-((3-(2,6- |
| | dioxopiperidin-3-yl)-4-oxo-3,4-dihydrobenzo[d][1,2,3]triazin-6-yl)oxy)acetyl)piperidin-4- |
| | yl)propanoyl)piperidin-4-yl)-1H-pyrazol-4-yl)pyrazine-2-carboxylate |
| TDH-438 | (R)-2-((4-fluorophenyl)amino)-2-oxo-1-phenylethyl 3-amino-6-(1-(1-(3-(1-(1-(3-(2,6- |
| | dioxopiperidin-3-yl)-4-oxo-3,4-dihydrobenzo[d][1,2,3]triazin-6-yl)azetidine-3- |
| | carbonyl)piperidin-4-yl)propanoyl)piperidin-4-yl)-1H-pyrazol-4-yl)pyrazine-2-carboxylate |
| TDH-439 | (R)-2-((4-fluorophenyl)amino)-2-oxo-1-phenylethyl 3-amino-6-(1-(1-(3-(1-(3-(2-(2,6- |
| | dioxopiperidin-3-yl)-1-oxoisoindolin-4-yl)propanoyl)piperidin-4-yl)propanoyl)piperidin-4-yl)- |
| | 1H-pyrazol-4-yl)pyrazine-2-carboxylate |
| TDH-440 | (R)-2-((4-fluorophenyl)amino)-2-oxo-1-phenylethyl 3-amino-6-(1-(1-(3-(4-((1-(2-((2-(2,6- |
| | dioxopiperidin-3-yl)-1,3-dioxoisoindolin-5-yl)oxy)acetyl)piperidin-4-yl)methyl)piperazin-1- |
| | yl)propyl)piperidin-4-yl)-1H-pyrazol-4-yl)pyrazine-2-carboxylate |
| TDH-441 | (R)-2-((4-fluorophenyl)amino)-2-oxo-1-phenylethyl 3-amino-6-(1-(1-(3-(4-((1-((2-(2,6- |
| | dioxopiperidin-3-yl)-1,3-dioxoisoindolin-5-yl)glycyl)piperidin-4-yl)methyl)piperazin-1- |
| | yl)propyl)piperidin-4-yl)-1H-pyrazol-4-yl)pyrazine-2-carboxylate |
| TDH-442 | (R)-2-((4-fluorophenyl)amino)-2-oxo-1-phenylethyl 3-amino-6-(1-(1-(7-((1-(2-((2-(2,6- |
| | dioxopiperidin-3-yl)-1,3-dioxoisoindolin-5-yl)oxy)acetyl)piperidin-4-yl)methyl)-7- |
| | azaspiro[3.5]nonan-2-yl)piperidin-4-yl)-1H-pyrazol-4-yl)pyrazine-2-carboxylate |
| TDH-443 | (R)-2-((4-fluorophenyl)amino)-2-oxo-1-phenylethyl 3-amino-6-(1-(1-(7-((1-((2-(2,6- |
| | dioxopiperidin-3-yl)-1,3-dioxoisoindolin-5-yl)glycyl)piperidin-4-yl)methyl)-7- |
| | azaspiro[3.5]nonan-2-yl)piperidin-4-yl)-1H-pyrazol-4-yl)pyrazine-2-carboxylate |
| TDH-445 | (R)-2-((4-fluorophenyl)amino)-2-oxo-1-phenylethyl 3-amino-6-(1-(1-((1-(3-(2,4- |
| | dioxotetrahydropyrimidin-1(2H)-yl)-4-methoxybenzoyl)piperidin-4-yl)methyl)piperidin-4-yl)- |
| | 1H-pyrazol-4-yl)pyrazine-2-carboxylate |
| TDH-446 | (R)-2-((4-fluorophenyl)amino)-2-oxo-1-phenylethyl 3-amino-6-(1-(1-(2-(1-(3-(2,4- |
| | dioxotetrahydropyrimidin-1(2H)-yl)-4-methoxybenzoyl)piperidin-4-yl)ethyl)piperidin-4-yl)- |
| | 1H-pyrazol-4-yl)pyrazine-2-carboxylate |
| TDH-447 | (R)-2-((4-fluorophenyl)amino)-2-oxo-1-phenylethyl 3-amino-6-(1-(1-(3-(4-(3-(2,4- |
| | dioxotetrahydropyrimidin-1(2H)-yl)-4-methoxybenzoyl)piperazin-1-yl)propyl)piperidin-4-yl)- |
| | 1H-pyrazol-4-yl)pyrazine-2-carboxylate |
| TDH-448 | (R)-2-((4-fluorophenyl)amino)-2-oxo-1-phenylethyl 3-amino-6-(1-(1-(2-((1-(3-(2,4- |
| | dioxotetrahydropyrimidin-1(2H)-yl)-4-methoxybenzoyl)piperidin-4-yl)oxy)ethyl)piperidin-4- |
| | yl)-1H-pyrazol-4-yl)pyrazine-2-carboxylate |
| TDH-450 | (R)-2-((4-fluorophenyl)amino)-2-oxo-1-phenylethyl 3-amino-6-(1-(1-((1-(2-(1-(2-(1-methyl- |
| | 2,6-dioxopiperidin-3-yl)-1,3-dioxoisoindolin-5-yl)piperidin-4-yl)acetyl)piperidin-4- |
| | yl)methyl)piperidin-4-yl)-1H-pyrazol-4-yl)pyrazine-2-carboxylate |
| TDH-451 | (R)-2-((4-fluorophenyl)amino)-2-oxo-1-phenylethyl 3-amino-6-(1-(1-(2-(1-(1-(2-((2-(2,6- |
| | dioxopiperidin-3-yl)-1,3-dioxoisoindolin-5-yl)oxy)acetyl)piperidin-4-yl)-1H-1,2,3-triazol-4- |
| | yl)ethyl)piperidin-4-yl)-1H-pyrazol-4-yl)pyrazine-2-carboxylate |
| TDH-452 | (R)-2-((4-fluorophenyl)amino)-2-oxo-1-phenylethyl 3-amino-6-(1-(1-(2-(1-(1-((2-(2,6- |
| | dioxopiperidin-3-yl)-1,3-dioxoisoindolin-5-yl)glycyl)piperidin-4-yl)-1H-1,2,3-triazol-4- |
| | yl)ethyl)piperidin-4-yl)-1H-pyrazol-4-yl)pyrazine-2-carboxylate |
| TDH-453 | (R)-2-((4-fluorophenyl)amino)-2-oxo-1-phenylethyl 3-amino-6-(1-(1-(3-(5-(2-((2-(2,6- |
| | dioxopiperidin-3-yl)-1,3-dioxoisoindolin-5-yl)oxy)acetyl)-2,5-diazabicyclo[2.2.1]heptan-2- |
| | yl)propyl)piperidin-4-yl)-1H-pyrazol-4-yl)pyrazine-2-carboxylate |
| TDH-454 | (R)-2-((4-fluorophenyl)amino)-2-oxo-1-phenylethyl 3-amino-6-(1-(1-(3-(5-((2-(2,6- |
| | dioxopiperidin-3-yl)-1,3-dioxoisoindolin-5-yl)glycyl)-2,5-diazabicyclo[2.2.1]heptan-2- |
| | yl)propyl)piperidin-4-yl)-1H-pyrazol-4-yl)pyrazine-2-carboxylate |
| TDH-466 | (R)-2-((4-fluorophenyl)amino)-2-oxo-1-phenylethyl 3-amino-6-(1-(1-(3-(5-(2-((2-(2,6- |
| | dioxopiperidin-3-yl)-1,3-dioxoisoindolin-5-yl)oxy)acetyl)hexahydropyrrolo[3,4-c]pyrrol- |
| | 2(1H)-yl)propyl)piperidin-4-yl)-1H-pyrazol-4-yl)pyrazine-2-carboxylate |
| TDH-467 | (R)-2-((4-fluorophenyl)amino)-2-oxo-1-phenylethyl 3-amino-6-(1-(1-(3-(5-((2-(2,6- |
| | dioxopiperidin-3-yl)-1,3-dioxoisoindolin-5-yl)glycyl)hexahydropyrrolo[3,4-c]pyrrol-2(1H)- |
| | yl)propyl)piperidin-4-yl)-1H-pyrazol-4-yl)pyrazine-2-carboxylate |
| TDH-472 | (R)-2-((4-fluorophenyl)amino)-2-oxo-1-phenylethyl 3-amino-6-(1-(1-(7-((1-(2-(2,6- |
| | dioxopiperidin-3-yl)-1,3-dioxoisoindolin-5-yl)piperidin-4-yl)methyl)-7-azaspiro[3.5]nonan-2- |
| | yl)piperidin-4-yl)-1H-pyrazol-4-yl)pyrazine-2-carboxylate |
| TDH-473 | (R)-2-((4-fluorophenyl)amino)-2-oxo-1-phenylethyl 3-amino-6-(1-(1-((1-((1-(2-(2,6- |
| | dioxopiperidin-3-yl)-1,3-dioxoisoindolin-5-yl)piperidin-4-yl)methyl)piperidin-4- |
| | yl)methyl)piperidin-4-yl)-1H-pyrazol-4-yl)pyrazine-2-carboxylate |
| TDH-474 | (R)-2-((4-fluorophenyl)amino)-2-oxo-1-phenylethyl 3-amino-6-(1-(1-(3-(4-((1-(2-(2,6- |
| | dioxopiperidin-3-yl)-1,3-dioxoisoindolin-5-yl)piperidin-4-yl)methyl)piperazin-1- |
| | yl)propyl)piperidin-4-yl)-1H-pyrazol-4-yl)pyrazine-2-carboxylate |
| TDH-475 | (R)-2-((4-fluorophenyl)amino)-2-oxo-1-phenylethyl 3-amino-6-(1-(1-(2-(1-(3-(2-(2,6- |
| | dioxopiperidin-3-yl)-1-oxoisoindolin-5-yl)propanoyl)piperidin-4-yl)ethyl)piperidin-4-yl)-1H- |
| | pyrazol-4-yl)pyrazine-2-carboxylate |
| TDH-476 | (R)-2-((4-fluorophenyl)amino)-2-oxo-1-phenylethyl 3-amino-6-(1-(1-(3-(4-(3-(2-(2,6- |
| | dioxopiperidin-3-yl)-1-oxoisoindolin-5-yl)propanoyl)piperazin-1-yl)propyl)piperidin-4-yl)-1H- |
| | pyrazol-4-yl)pyrazine-2-carboxylate |
| TDH-477 | (R)-2-((4-fluorophenyl)amino)-2-oxo-1-phenylethyl 3-amino-6-(1-(1-((1-((1-(3-(2,4- |
| | dioxotetrahydropyrimidin-1(2H)-yl)-4-methoxybenzoyl)piperidin-4-yl)methyl)piperidin-4- |
| | yl)methyl)piperidin-4-yl)-1H-pyrazol-4-yl)pyrazine-2-carboxylate |
| TDH-478 | (R)-2-((4-fluorophenyl)amino)-2-oxo-1-phenylethyl 3-amino-6-(1-(1-(3-(4-((1-(3-(2,4- |
| | dioxotetrahydropyrimidin-1(2H)-yl)-4-methoxybenzoyl)piperidin-4-yl)methyl)piperazin-1- |
| | yl)propyl)piperidin-4-yl)-1H-pyrazol-4-yl)pyrazine-2-carboxylate |
| TDH-480 | (R)-2-((4-fluorophenyl)amino)-2-oxo-1-phenylethyl 3-amino-6-(1-(1-(3-(5-(3-(2-(2,6- |
| | dioxopiperidin-3-yl)-1,3-dioxoisoindolin-5-yl)propanoyl)hexahydropyrrolo[3,4-c]pyrrol- |
| | 2(1H)-yl)propyl)piperidin-4-yl)-1H-pyrazol-4-yl)pyrazine-2-carboxylate |
| TDH-481 | (R)-2-((4-fluorophenyl)amino)-2-oxo-1-phenylethyl 3-amino-6-(1-(1-(3-(1-(2-(2,6- |
| | dioxopiperidin-3-yl)-1,3-dioxoisoindolin-5-yl)piperidin-4-yl)propyl)piperidin-4-yl)-1H- |
| | pyrazol-4-yl)pyrazine-2-carboxylate |
| TDH-482 | (R)-2-((4-fluorophenyl)amino)-2-oxo-1-phenylethyl 3-amino-6-(1-(1-(3-(1-(2-((2-(2,6- |
| | dioxopiperidin-3-yl)-1,3-dioxoisoindolin-5-yl)oxy)acetyl)piperidin-4-yl)propyl)piperidin-4-yl)- |
| | 1H-pyrazol-4-yl)pyrazine-2-carboxylate |
| TDH-483 | (R)-2-((4-fluorophenyl)amino)-2-oxo-1-phenylethyl 3-amino-6-(1-(1-(3-(1-((2-(2,6- |
| | dioxopiperidin-3-yl)-1,3-dioxoisoindolin-5-yl)glycyl)piperidin-4-yl)propyl)piperidin-4-yl)-1H- |
| | pyrazol-4-yl)pyrazine-2-carboxylate |
| TDH-484 | (R)-2-((4-fluorophenyl)amino)-2-oxo-1-phenylethyl 3-amino-6-(1-(1-(3-(1-(3-(2-(2,6- |
| | dioxopiperidin-3-yl)-1,3-dioxoisoindolin-5-yl)propanoyl)piperidin-4-yl)propyl)piperidin-4-yl)- |
| | 1H-pyrazol-4-yl)pyrazine-2-carboxylate |
| TDH-485 | (R)-2-((3-chlorophenyl)amino)-2-oxo-1-phenylethyl 3-amino-6-(1-(1-(3-(4-(2-((2-(2,6- |
| | dioxopiperidin-3-yl)-1,3-dioxoisoindolin-5-yl)oxy)acetyl)piperazin-1-yl)propyl)piperidin-4- |
| | yl)-1H-pyrazol-4-yl)pyrazine-2-carboxylate |
| TDH-486 | (R)-2-((3-methoxyphenyl)amino)-2-oxo-1-phenylethyl 3-amino-6-(1-(1-(3-(4-(2-((2-(2,6- |
| | dioxopiperidin-3-yl)-1,3-dioxoisoindolin-5-yl)oxy)acetyl)piperazin-1-yl)propyl)piperidin-4- |
| | yl)-1H-pyrazol-4-yl)pyrazine-2-carboxylate |
| TDH-487 | (R)-2-oxo-1-phenyl-2-(m-tolylamino)ethyl 3-amino-6-(1-(1-(3-(4-(2-((2-(2,6-dioxopiperidin- |
| | 3-yl)-1,3-dioxoisoindolin-5-yl)oxy)acetyl)piperazin-1-yl)propyl)piperidin-4-yl)-1H-pyrazol-4- |
| | yl)pyrazine-2-carboxylate |
| TDH-491 | 3-amino-6-(1-(1-(3-(4-(2-(2,6-dioxopiperidin-3-yl)-1,3-dioxoisoindolin-5-yl)piperazin-1- |
| | yl)propyl)piperidin-4-yl)-1H-pyrazol-4-yl)-N-((R)-2-((4-fluorophenyl)amino)-2-oxo-1- |
| | phenylethyl)pyrazine-2-carboxamide |
| TDH-492 | 3-amino-6-(1-(1-(3-(4-(2-((2-(2,6-dioxopiperidin-3-yl)-1,3-dioxoisoindolin-5- |
| | yl)oxy)acetyl)piperazin-1-yl)propyl)piperidin-4-yl)-1H-pyrazol-4-yl)-N-((R)-2-((4- |
| | fluorophenyl)amino)-2-oxo-1-phenylethyl)pyrazine-2-carboxamide |
| TDH-493 | 3-amino-6-(1-(1-(3-(4-((2-(2,6-dioxopiperidin-3-yl)-1,3-dioxoisoindolin-5-yl)glycyl)piperazin- |
| | 1-yl)propyl)piperidin-4-yl)-1H-pyrazol-4-yl)-N-((R)-2-((4-fluorophenyl)amino)-2-oxo-1- |
| | phenylethyl)pyrazine-2-carboxamide |
| TDH-494 | (R)-1-(2-chlorophenyl)-2-((4-fluorophenyl)amino)-2-oxoethyl 3-amino-6-(1-(1-(3-(4-(2-((2- |
| | (2,6-dioxopiperidin-3-yl)-1,3-dioxoisoindolin-5-yl)oxy)acetyl)piperazin-1-yl)propyl)piperidin- |
| | 4-yl)-1H-pyrazol-4-yl)pyrazine-2-carboxylate |
| TDH-495 | (R)-2-((4-fluorophenyl)amino)-2-oxo-1-phenylethyl 3-amino-6-(1-(1-(3-(4-(2-(3-((1-(2,6- |
| | dioxopiperidin-3-yl)-2,5-dioxo-2,5-dihydro-1H-pyrrol-3-yl)amino)phenyl)acetyl)piperazin-1- |
| | yl)propyl)piperidin-4-yl)-1H-pyrazol-4-yl)pyrazine-2-carboxylate |
| TDH-496 | (R)-2-((4-fluorophenyl)amino)-2-oxo-1-phenylethyl 3-amino-6-(1-(1-(2-((1-(2-(3-((1-(2,6- |
| | dioxopiperidin-3-yl)-2,5-dioxo-2,5-dihydro-1H-pyrrol-3-yl)amino)phenyl)acetyl)piperidin-4- |
| | yl)oxy)ethyl)piperidin-4-yl)-1H-pyrazol-4-yl)pyrazine-2-carboxylate |
| TDH-497 | (R)-2-((4-fluorophenyl)amino)-2-oxo-1-phenylethyl 3-amino-6-(1-(1-(((1r,4r)-4-((3-(2,4- |
| | dioxotetrahydropyrimidin-1(2H)-yl)-4- |
| | methoxybenzamido)methyl)cyclohexyl)methyl)piperidin-4-yl)-1H-pyrazol-4-yl)pyrazine-2- |
| | carboxylate |
| TDH-498 | (R)-2-((4-fluorophenyl)amino)-2-oxo-1-phenylethyl 3-amino-6-(1-(1-(((1r,4r)-4-((2-(3-((1- |
| | (2,6-dioxopiperidin-3-yl)-2,5-dioxo-2,5-dihydro-1H-pyrrol-3- |
| | yl)amino)phenyl)acetamido)methyl)cyclohexyl)methyl)piperidin-4-yl)-1H-pyrazol-4- |
| | yl)pyrazine-2-carboxylate |
| TDH-499 | (R)-2-((4-fluorophenyl)amino)-2-oxo-1-phenylethyl 3-amino-6-(1-(1-(7-((1-(3-(2,4- |
| | dioxotetrahydropyrimidin-1(2H)-yl)-4-methoxybenzoyl)piperidin-4-yl)methyl)-7- |
| | azaspiro[3.5]nonan-2-yl)piperidin-4-yl)-1H-pyrazol-4-yl)pyrazine-2-carboxylate |
| TDH-500 | (R)-2-((4-fluorophenyl)amino)-2-oxo-1-phenylethyl 3-amino-6-(1-(1-(7-((1-(2-(3-((1-(2,6- |
| | dioxopiperidin-3-yl)-2,5-dioxo-2,5-dihydro-1H-pyrrol-3-yl)amino)phenyl)acetyl)piperidin-4- |
| | yl)methyl)-7-azaspiro[3.5]nonan-2-yl)piperidin-4-yl)-1H-pyrazol-4-yl)pyrazine-2- |
| | carboxylate |
| TDH-501 | (R)-2-((4-fluorophenyl)amino)-2-oxo-1-phenylethyl 3-amino-6-(1-(1-((1-(2-(1-(3-(2,4- |
| | dioxotetrahydropyrimidin-1(2H)-yl)-4-methoxybenzoyl)piperidin-4-yl)ethyl)piperidin-4- |
| | yl)methyl)piperidin-4-yl)-1H-pyrazol-4-yl)pyrazine-2-carboxylate |
| TDH-502 | (R)-2-((4-fluorophenyl)amino)-2-oxo-1-phenylethyl 3-amino-6-(1-(1-((1-(2-(1-(1-(3-(2,4- |
| | dioxotetrahydropyrimidin-1(2H)-yl)-4-methoxybenzoyl)piperidine-4-carbonyl)piperidin-4- |
| | yl)ethyl)piperidin-4-yl)methyl)piperidin-4-yl)-1H-pyrazol-4-yl)pyrazine-2-carboxylate |
| TDH-503 | (R)-2-((4-fluorophenyl)amino)-2-oxo-1-phenylethyl 3-amino-6-(1-(1-((1-(7-(3-(2,4- |
| | dioxotetrahydropyrimidin-1(2H)-yl)-4-methoxybenzoyl)-7-azaspiro[3.5]nonan-2- |
| | yl)piperidin-4-yl)methyl)piperidin-4-yl)-1H-pyrazol-4-yl)pyrazine-2-carboxylate |
| TDH-504 | (R)-2-((4-fluorophenyl)amino)-2-oxo-1-phenylethyl 3-amino-6-(1-(1-(2-(1-(7-(3-(2,4- |
| | dioxotetrahydropyrimidin-1(2H)-yl)-4-methoxybenzoyl)-7-azaspiro[3.5]nonan-2- |
| | yl)piperidin-4-yl)ethyl)piperidin-4-yl)-1H-pyrazol-4-yl)pyrazine-2-carboxylate |
| TDH-505 | (R)-2-((4-fluorophenyl)amino)-2-oxo-1-phenylethyl 3-amino-6-(1-(1-(2-(1-((1-(3-(2,4- |
| | dioxotetrahydropyrimidin-1(2H)-yl)-4-methoxybenzoyl)piperidin-4-yl)methyl)piperidin-4- |
| | yl)ethyl)piperidin-4-yl)-1H-pyrazol-4-yl)pyrazine-2-carboxylate |
| TDH-506 | (R)-2-((4-fluorophenyl)amino)-2-oxo-1-phenylethyl 3-amino-6-(1-(1-((1-(2-((1-(3-(2,4- |
| | dioxotetrahydropyrimidin-1(2H)-yl)-4-methoxybenzoyl)piperidin-4-yl)oxy)ethyl)piperidin-4- |
| | yl)methyl)piperidin-4-yl)-1H-pyrazol-4-yl)pyrazine-2-carboxylate |
| TDH-507 | (R)-2-((4-fluorophenyl)amino)-2-oxo-1-phenylethyl 3-amino-6-(1-(1-((1-(2-(1-(2-(3-((1-(2,6- |
| | dioxopiperidin-3-yl)-2,5-dioxo-2,5-dihydro-1H-pyrrol-3-yl)amino)phenyl)acetyl)piperidin-4- |
| | yl)ethyl)piperidin-4-yl)methyl)piperidin-4-yl)-1H-pyrazol-4-yl)pyrazine-2-carboxylate |
| TDH-508 | (R)-2-((4-fluorophenyl)amino)-2-oxo-1-phenylethyl 3-amino-6-(1-(1-((1-(7-(1-(3-(2,4- |
| | dioxotetrahydropyrimidin-1(2H)-yl)-4-methoxybenzoyl)piperidine-4-carbonyl)-7- |
| | azaspiro[3.5]nonan-2-yl)piperidin-4-yl)methyl)piperidin-4-yl)-1H-pyrazol-4-yl)pyrazine-2- |
| | carboxylate |
| TDH-509 | (R)-2-((4-fluorophenyl)amino)-2-oxo-1-phenylethyl 3-amino-6-(1-(1-(2-(1-(7-(2-(3-((1-(2,6- |
| | dioxopiperidin-3-yl)-2,5-dioxo-2,5-dihydro-1H-pyrrol-3-yl)amino)phenyl)acetyl)-7- |
| | azaspiro[3.5]nonan-2-yl)piperidin-4-yl)ethyl)piperidin-4-yl)-1H-pyrazol-4-yl)pyrazine-2- |
| | carboxylate |
| TDH-510 | (R)-2-((4-fluorophenyl)amino)-2-oxo-1-phenylethyl 3-amino-6-(1-(1-(2-(1-((1-(2-(3-((1-(2,6- |
| | dioxopiperidin-3-yl)-2,5-dioxo-2,5-dihydro-1H-pyrrol-3-yl)amino)phenyl)acetyl)piperidin-4- |
| | yl)methyl)piperidin-4-yl)ethyl)piperidin-4-yl)-1H-pyrazol-4-yl)pyrazine-2-carboxylate |
| TDH-511 | (R)-2-((4-fluorophenyl)amino)-2-oxo-1-phenylethyl 3-amino-6-(1-(1-(2-(1-((1-(1-(3-(2,4- |
| | dioxotetrahydropyrimidin-1(2H)-yl)-4-methoxybenzoyl)piperidine-4-carbonyl)piperidin-4- |
| | yl)methyl)piperidin-4-yl)ethyl)piperidin-4-yl)-1H-pyrazol-4-yl)pyrazine-2-carboxylate |
| TDH-512 | (R)-2-((4-fluorophenyl)amino)-2-oxo-1-phenylethyl 3-amino-6-(1-(1-((1-(2-((1-(1-(3-(2,4- |
| | dioxotetrahydropyrimidin-1(2H)-yl)-4-methoxybenzoyl)piperidine-4-carbonyl)piperidin-4- |
| | yl)oxy)ethyl)piperidin-4-yl)methyl)piperidin-4-yl)-1H-pyrazol-4-yl)pyrazine-2-carboxylate |
| TDH-513 | (R)-2-((4-fluorophenyl)amino)-2-oxo-1-phenylethyl 3-amino-6-(1-(1-((1-(2-(1-(2-(2,6- |
| | dioxopiperidin-3-yl)-6-fluoro-1,3-dioxoisoindolin-5-yl)piperidin-4-yl)ethyl)piperidin-4- |
| | yl)methyl)piperidin-4-yl)-1H-pyrazol-4-yl)pyrazine-2-carboxylate |
| TDH-514 | (R)-2-((4-fluorophenyl)amino)-2-oxo-1-phenylethyl 3-amino-6-(1-(1-(2-(1-((1-(2-(2,6- |
| | dioxopiperidin-3-yl)-6-fluoro-1,3-dioxoisoindolin-5-yl)piperidin-4-yl)methyl)piperidin-4- |
| | yl)ethyl)piperidin-4-yl)-1H-pyrazol-4-yl)pyrazine-2-carboxylate |
| TDH-515 | (R)-2-((4-fluorophenyl)amino)-2-oxo-1-phenylethyl 3-amino-6-(1-(1-((1-(1-(3-(2,4- |
| | dioxotetrahydropyrimidin-1(2H)-yl)-4-methoxybenzoyl)piperidine-4-carbonyl)piperidin-4- |
| | yl)methyl)piperidin-4-yl)-1H-pyrazol-4-yl)pyrazine-2-carboxylate |
| TDH-516 | (R)-2-((4-fluorophenyl)amino)-2-oxo-1-phenylethyl 3-amino-6-(1-(1-((1-(2-(3-((1-(2,6- |
| | dioxopiperidin-3-yl)-2,5-dioxo-2,5-dihydro-1H-pyrrol-3-yl)amino)phenyl)acetyl)piperidin-4- |
| | yl)methyl)piperidin-4-yl)-1H-pyrazol-4-yl)pyrazine-2-carboxylate |
| TDH-517 | (R)-2-((4-fluorophenyl)amino)-2-oxo-1-phenylethyl 3-amino-6-(1-(1-(2-(1-(1-(3-(2,4- |
| | dioxotetrahydropyrimidin-1(2H)-yl)-4-methoxybenzoyl)piperidine-4-carbonyl)piperidin-4- |
| | yl)ethyl)piperidin-4-yl)-1H-pyrazol-4-yl)pyrazine-2-carboxylate |
| TDH-518 | (R)-2-((4-fluorophenyl)amino)-2-oxo-1-phenylethyl 3-amino-6-(1-(1-(2-(1-(2-(3-((1-(2,6- |
| | dioxopiperidin-3-yl)-2,5-dioxo-2,5-dihydro-1H-pyrrol-3-yl)amino)phenyl)acetyl)piperidin-4- |
| | yl)ethyl)piperidin-4-yl)-1H-pyrazol-4-yl)pyrazine-2-carboxylate |
| TDH-519 | (R)-2-((4-fluorophenyl)amino)-2-oxo-1-phenylethyl 3-amino-6-(1-(1-((1-(2-(2,6- |
| | dioxopiperidin-3-yl)-6-fluoro-1,3-dioxoisoindolin-5-yl)piperidin-4-yl)methyl)piperidin-4-yl)- |
| | 1H-pyrazol-4-yl)pyrazine-2-carboxylate |
| TDH-520 | (R)-2-((4-fluorophenyl)amino)-2-oxo-1-phenylethyl 3-amino-6-(1-(1-(2-(1-(2-(2,6- |
| | dioxopiperidin-3-yl)-6-fluoro-1,3-dioxoisoindolin-5-yl)piperidin-4-yl)ethyl)piperidin-4-yl)-1H- |
| | pyrazol-4-yl)pyrazine-2-carboxylate |
| TDH-521 | (R)-2-((4-fluorophenyl)amino)-2-oxo-1-phenylethyl 3-amino-6-(1-(1-((1-(1-(2,6- |
| | dioxopiperidin-3-yl)-2,5-dioxo-2,5-dihydro-1H-pyrrol-3-yl)piperidin-4-yl)methyl)piperidin-4- |
| | yl)-1H-pyrazol-4-yl)pyrazine-2-carboxylate |
| TDH-522 | (R)-2-((4-fluorophenyl)amino)-2-oxo-1-phenylethyl 3-amino-6-(1-(1-(2-(1-(1-(2,6- |
| | dioxopiperidin-3-yl)-2,5-dioxo-2,5-dihydro-1H-pyrrol-3-yl)piperidin-4-yl)ethyl)piperidin-4-yl)- |
| | 1H-pyrazol-4-yl)pyrazine-2-carboxylate |
| TDH-523 | (R)-2-((4-fluorophenyl)amino)-2-oxo-1-phenylethyl 3-amino-6-(1-(1-((1-(1-(1-(2,6- |
| | dioxopiperidin-3-yl)-2,5-dioxo-2,5-dihydro-1H-pyrrol-3-yl)piperidine-4-carbonyl)piperidin-4- |
| | yl)methyl)piperidin-4-yl)-1H-pyrazol-4-yl)pyrazine-2-carboxylate |
| TDH-524 | (R)-2-((4-fluorophenyl)amino)-2-oxo-1-phenylethyl 3-amino-6-(1-(1-(2-(1-(1-(1-(2,6- |
| | dioxopiperidin-3-yl)-2,5-dioxo-2,5-dihydro-1H-pyrrol-3-yl)piperidine-4-carbonyl)piperidin-4- |
| | yl)ethyl)piperidin-4-yl)-1H-pyrazol-4-yl)pyrazine-2-carboxylate |
| TDH-525 | (R)-2-((4-fluorophenyl)amino)-2-oxo-1-phenylethyl 3-amino-6-(1-(1-(3-(4-(2-((2-(1-methyl- |
| | 2,6-dioxopiperidin-3-yl)-1,3-dioxoisoindolin-5-yl)oxy)acetyl)piperazin-1-yl)propyl)piperidin- |
| | 4-yl)-1H-pyrazol-4-yl)pyrazine-2-carboxylate |
| TDH-526 | (R)-2-((4-fluorophenyl)amino)-2-oxo-1-phenylethyl 3-amino-6-(1-(1-(3-(4-(1-(1-(2,6- |
| | dioxopiperidin-3-yl)-2,5-dioxo-2,5-dihydro-1H-pyrrol-3-yl)piperidine-4-carbonyl)piperazin-1- |
| | yl)propyl)piperidin-4-yl)-1H-pyrazol-4-yl)pyrazine-2-carboxylate |
| TDH-527 | (R)-2-((4-fluorophenyl)amino)-2-oxo-1-phenylethyl 3-amino-6-(1-(1-((1-((1-(1-(1-(2,6- |
| | dioxopiperidin-3-yl)-2,5-dioxo-2,5-dihydro-1H-pyrrol-3-yl)piperidine-4-carbonyl)piperidin-4- |
| | yl)methyl)piperidin-4-yl)methyl)piperidin-4-yl)-1H-pyrazol-4-yl)pyrazine-2-carboxylate |
| TDH-528 | (R)-2-((4-fluorophenyl)amino)-2-oxo-1-phenylethyl 3-amino-6-(1-(1-(7-((1-(1-(1-(2,6- |
| | dioxopiperidin-3-yl)-2,5-dioxo-2,5-dihydro-1H-pyrrol-3-yl)piperidine-4-carbonyl)piperidin-4- |
| | yl)methyl)-7-azaspiro[3.5]nonan-2-yl)piperidin-4-yl)-1H-pyrazol-4-yl)pyrazine-2- |
| | carboxylate |
| TDH-529 | (R)-2-((4-fluorophenyl)amino)-2-oxo-1-phenylethyl 3-amino-6-(1-(1-(2-((1-(1-(1-(2,6- |
| | dioxopiperidin-3-yl)-2,5-dioxo-2,5-dihydro-1H-pyrrol-3-yl)piperidine-4-carbonyl)piperidin-4- |
| | yl)oxy)ethyl)piperidin-4-yl)-1H-pyrazol-4-yl)pyrazine-2-carboxylate |
| TDH-530 | (R)-2-((4-fluorophenyl)amino)-2-oxo-1-phenylethyl 3-amino-6-(1-(1-(3-(4-(2-(2,6- |
| | dioxopiperidin-3-yl)-1-oxoisoindoline-5-carbonyl)piperazin-1-yl)propyl)piperidin-4-yl)-1H- |
| | pyrazol-4-yl)pyrazine-2-carboxylate |
| TDH-531 | (R)-2-((4-fluorophenyl)amino)-2-oxo-1-phenylethyl 3-amino-6-(1-(1-(2-(1-(1-(1-(2,6- |
| | dioxopiperidin-3-yl)-2,5-dioxo-2,5-dihydro-1H-pyrrol-3-yl)azetidine-3-carbonyl)piperidin-4- |
| | yl)ethyl)piperidin-4-yl)-1H-pyrazol-4-yl)pyrazine-2-carboxylate |
| TDH-532 | (R)-2-((4-fluorophenyl)amino)-2-oxo-1-phenylethyl 3-amino-6-(1-(1-(3-(4-(1-(1-(2,6- |
| | dioxopiperidin-3-yl)-2,5-dioxo-2,5-dihydro-1H-pyrrol-3-yl)azetidine-3-carbonyl)piperazin-1- |
| | yl)propyl)piperidin-4-yl)-1H-pyrazol-4-yl)pyrazine-2-carboxylate |
| TDH-533 | (R)-2-((4-fluorophenyl)amino)-2-oxo-1-phenylethyl 3-amino-6-(1-(1-((1-((1-(1-(1-(2,6- |
| | dioxopiperidin-3-yl)-2,5-dioxo-2,5-dihydro-1H-pyrrol-3-yl)azetidine-3-carbonyl)piperidin-4- |
| | yl)methyl)piperidin-4-yl)methyl)piperidin-4-yl)-1H-pyrazol-4-yl)pyrazine-2-carboxylate |
| TDH-534 | (R)-2-((4-fluorophenyl)amino)-2-oxo-1-phenylethyl 3-amino-6-(1-(1-(7-((1-(1-(1-(2,6- |
| | dioxopiperidin-3-yl)-2,5-dioxo-2,5-dihydro-1H-pyrrol-3-yl)azetidine-3-carbonyl)piperidin-4- |
| | yl)methyl)-7-azaspiro[3.5]nonan-2-yl)piperidin-4-yl)-1H-pyrazol-4-yl)pyrazine-2- |
| | carboxylate |
| TDH-535 | (R)-2-((4-fluorophenyl)amino)-2-oxo-1-phenylethyl 3-amino-6-(1-(1-(3-(4-(4-((2,6- |
| | dioxopiperidin-3-yl)carbamoyl)benzoyl)piperazin-1-yl)propyl)piperidin-4-yl)-1H-pyrazol-4- |
| | yl)pyrazine-2-carboxylate |
| TDH-536 | (R)-2-((4-fluorophenyl)amino)-2-oxo-1-phenylethyl 3-amino-6-(1-(1-(2-(1-(4-((2,6- |
| | dioxopiperidin-3-yl)carbamoyl)benzoyl)piperidin-4-yl)ethyl)piperidin-4-yl)-1H-pyrazol-4- |
| | yl)pyrazine-2-carboxylate |
| TDH-537 | (R)-2-((4-fluorophenyl)amino)-2-oxo-1-phenylethyl 3-amino-6-(1-(1-(7-((1-(4-((2,6- |
| | dioxopiperidin-3-yl)carbamoyl)benzoyl)piperidin-4-yl)methyl)-7-azaspiro[3.5]nonan-2- |
| | yl)piperidin-4-yl)-1H-pyrazol-4-yl)pyrazine-2-carboxylate |
| TDH-538 | (R)-2-((4-fluorophenyl)amino)-2-oxo-1-phenylethyl 3-amino-6-(1-(1-(2-((1-(4-((2,6- |
| | dioxopiperidin-3-yl)carbamoyl)benzoyl)piperidin-4-yl)oxy)ethyl)piperidin-4-yl)-1H-pyrazol- |
| | 4-yl)pyrazine-2-carboxylate |
| TDH-539 | (R)-2-((4-fluorophenyl)amino)-2-oxo-1-phenylethyl 3-amino-6-(1-(1-((1-((1-(4-((2,6- |
| | dioxopiperidin-3-yl)carbamoyl)benzoyl)piperidin-4-yl)methyl)piperidin-4- |
| | yl)methyl)piperidin-4-yl)-1H-pyrazol-4-yl)pyrazine-2-carboxylate |
| TDH-540 | (R)-2-((4-fluorophenyl)amino)-2-oxo-1-phenylethyl 3-amino-6-(1-(1-(3-(4-(1-(1-(2,6- |
| | dioxopiperidin-3-yl)-3-methyl-2-oxo-2,3-dihydro-1H-benzo[d]imidazol-5-yl)piperidine-4- |
| | carbonyl)piperazin-1-yl)propyl)piperidin-4-yl)-1H-pyrazol-4-yl)pyrazine-2-carboxylate |
| TDH-541 | (R)-2-((4-fluorophenyl)amino)-2-oxo-1-phenylethyl 3-amino-6-(1-(1-(2-((1-(1-(1-(2,6- |
| | dioxopiperidin-3-yl)-2,5-dioxo-2,5-dihydro-1H-pyrrol-3-yl)azetidine-3-carbonyl)piperidin-4- |
| | yl)oxy)ethyl)piperidin-4-yl)-1H-pyrazol-4-yl)pyrazine-2-carboxylate |
| TDH-542 | (R)-2-((4-fluorophenyl)amino)-2-oxo-1-phenylethyl 3-amino-6-(1-(1-(3-(4-(3-((2,6- |
| | dioxopiperidin-3-yl)carbamoyl)benzoyl)piperazin-1-yl)propyl)piperidin-4-yl)-1H-pyrazol-4- |
| | yl)pyrazine-2-carboxylate |
| TDH-543 | (R)-2-((4-fluorophenyl)amino)-2-oxo-1-phenylethyl 3-amino-6-(1-(1-(2-(1-(3-((2,6- |
| | dioxopiperidin-3-yl)carbamoyl)benzoyl)piperidin-4-yl)ethyl)piperidin-4-yl)-1H-pyrazol-4- |
| | yl)pyrazine-2-carboxylate |
| TDH-544 | (R)-2-((4-fluorophenyl)amino)-2-oxo-1-phenylethyl 3-amino-6-(1-(1-(7-((1-(3-((2,6- |
| | dioxopiperidin-3-yl)carbamoyl)benzoyl)piperidin-4-yl)methyl)-7-azaspiro[3.5]nonan-2- |
| | yl)piperidin-4-yl)-1H-pyrazol-4-yl)pyrazine-2-carboxylate |
| TDH-545 | (R)-2-((4-fluorophenyl)amino)-2-oxo-1-phenylethyl 3-amino-6-(1-(1-(2-((1-(3-((2,6- |
| | dioxopiperidin-3-yl)carbamoyl)benzoyl)piperidin-4-yl)oxy)ethyl)piperidin-4-yl)-1H-pyrazol- |
| | 4-yl)pyrazine-2-carboxylate |
| TDH-546 | (R)-2-((4-fluorophenyl)amino)-2-oxo-1-phenylethyl 3-amino-6-(1-(1-((1-((1-(3-((2,6- |
| | dioxopiperidin-3-yl)carbamoyl)benzoyl)piperidin-4-yl)methyl)piperidin-4- |
| | yl)methyl)piperidin-4-yl)-1H-pyrazol-4-yl)pyrazine-2-carboxylate |
| TDH-547 | (R)-2-((4-fluorophenyl)amino)-2-oxo-1-phenylethyl 3-amino-6-(1-(1-(2-(1-(2-(2,6- |
| | dioxopiperidin-3-yl)-1-oxoisoindoline-5-carbonyl)piperidin-4-yl)ethyl)piperidin-4-yl)-1H- |
| | pyrazol-4-yl)pyrazine-2-carboxylate |
| TDH-548 | (R)-2-((4-fluorophenyl)amino)-2-oxo-1-phenylethyl 3-amino-6-(1-(1-(7-((1-(2-(2,6- |
| | dioxopiperidin-3-yl)-1-oxoisoindoline-5-carbonyl)piperidin-4-yl)methyl)-7- |
| | azaspiro[3.5]nonan-2-yl)piperidin-4-yl)-1H-pyrazol-4-yl)pyrazine-2-carboxylate |
| TDH-549 | (R)-2-((4-fluorophenyl)amino)-2-oxo-1-phenylethyl 3-amino-6-(1-(1-(2-((1-(2-(2,6- |
| | dioxopiperidin-3-yl)-1-oxoisoindoline-5-carbonyl)piperidin-4-yl)oxy)ethyl)piperidin-4-yl)-1H- |
| | pyrazol-4-yl)pyrazine-2-carboxylate |
| TDH-550 | (R)-2-((4-fluorophenyl)amino)-2-oxo-1-phenylethyl 3-amino-6-(1-(1-((1-((1-(2-(2,6- |
| | dioxopiperidin-3-yl)-1-oxoisoindoline-5-carbonyl)piperidin-4-yl)methyl)piperidin-4- |
| | yl)methyl)piperidin-4-yl)-1H-pyrazol-4-yl)pyrazine-2-carboxylate |
| TDH-551 | (R)-2-((4-fluorophenyl)amino)-2-oxo-1-phenylethyl 3-amino-6-(1-(1-(3-(4-(3-(2,4- |
| | dioxotetrahydropyrimidin-1(2H)-yl)benzofuran-6-carbonyl)piperazin-1-yl)propyl)piperidin-4- |
| | yl)-1H-pyrazol-4-yl)pyrazine-2-carboxylate |
| TDH-552 | (R)-2-((4-fluorophenyl)amino)-2-oxo-1-phenylethyl 3-amino-6-(1-(1-(2-(1-(3-(2,4- |
| | dioxotetrahydropyrimidin-1(2H)-yl)benzofuran-6-carbonyl)piperidin-4-yl)ethyl)piperidin-4- |
| | yl)-1H-pyrazol-4-yl)pyrazine-2-carboxylate |
| TDH-553 | (R)-2-((4-fluorophenyl)amino)-2-oxo-1-phenylethyl 3-amino-6-(1-(1-(7-((1-(3-(2,4- |
| | dioxotetrahydropyrimidin-1(2H)-yl)benzofuran-6-carbonyl)piperidin-4-yl)methyl)-7- |
| | azaspiro[3.5]nonan-2-yl)piperidin-4-yl)-1H-pyrazol-4-yl)pyrazine-2-carboxylate |
| TDH-554 | (R)-2-((4-fluorophenyl)amino)-2-oxo-1-phenylethyl 3-amino-6-(1-(1-(2-((1-(3-(2,4- |
| | dioxotetrahydropyrimidin-1(2H)-yl)benzofuran-6-carbonyl)piperidin-4- |
| | yl)oxy)ethyl)piperidin-4-yl)-1H-pyrazol-4-yl)pyrazine-2-carboxylate |
| TDH-555 | (R)-2-((4-fluorophenyl)amino)-2-oxo-1-phenylethyl 3-amino-6-(1-(1-((1-((1-(3-(2,4- |
| | dioxotetrahydropyrimidin-1(2H)-yl)benzofuran-6-carbonyl)piperidin-4-yl)methyl)piperidin-4- |
| | yl)methyl)piperidin-4-yl)-1H-pyrazol-4-yl)pyrazine-2-carboxylate |

21. A composition comprising a compound of claim 1 or a pharmaceutically acceptable salt thereof, and a pharmaceutically acceptable carrier.

22. A composition comprising a compound of claim 7 or a pharmaceutically acceptable salt thereof, and a pharmaceutically acceptable carrier.

23. A method for treating or preventing a disease related to MLKL (Mixed Lineage Kinase domain-Like protein), comprising
administering a therapeutically effective amount of the compound of claim 1 or a pharmaceutically acceptable salt thereof to a subject in need thereof.

24. The method of claim 23, wherein the disease related to MLKL is traumatic brain injury, stroke, bone marrow failure, (acute) pancreatitis, arteriosclerosis, metabolic disease, ischemia reperfusion injury, transplantation, infection, chronic obstructive pulmonary disease, remote lung injury, hepatotoxicity, (alcoholic and non-alcoholic) steatohepatitis, remote liver injury, autoimmune hepatitis, psoriasis, hypoxic ischemic encephalopathy, ovarian cancer, renal injury, fatty liver, inflammation, Crohn's colitis, ulcerative colitis, or ileitis.

25. A method for treating or preventing a disease related to MLKL (Mixed Lineage Kinase domain-Like protein), comprising administering a therapeutically effective amount of the compound of claim 7 or a pharmaceutically acceptable salt thereof to a subject in need thereof.

26. The method of claim 25, wherein the disease related to MLKL is traumatic brain injury, stroke, bone marrow failure, (acute) pancreatitis, arteriosclerosis, metabolic disease, ischemia reperfusion injury, transplantation, infection, chronic obstructive pulmonary disease, remote lung injury, hepatotoxicity, (alcoholic and non-alcoholic) steatohepatitis, remote liver injury, autoimmune hepatitis, psoriasis, hypoxic ischemic encephalopathy, ovarian cancer, renal injury, fatty liver, inflammation, Crohn's colitis, ulcerative colitis, or ileitis.

27. The method of claim 26, wherein the disease related to MLKL is non-alcoholic steatohepatitis, autoimmune hepatitis, psoriasis, hypoxic ischemic encephalopathy, stroke, ovarian cancer, renal damage, fatty liver or inflammation.
